(19) United States
(12) Patent Application Publication
(10) Pub. No.: US 2004/0076955 A1
(43) Pub. Date: Apr. 22, 2004

(54) Methods of diagnosis of bladder cancer, compositions and methods of screening for modulators of bladder cancer

(75) Inventors: David H. Mack, Menlo Park, CA (US); Natasha Aziz, Palo Alto, CA (US)

Correspondence Address: HOWREY SIMON ARNOLD & WHITE, LLP, BOX 34, 301 RAVENSWOOD AVE., MENLO PARK, CA, 94025, US

(73) Assignee: Eos Biotechnology, Inc., South San Francisco, CA (US)

(21) Appl. No.: 10/188,832

(22) Filed: Jul. 2, 2002

Related U.S. Application Data
(60) Provisional application No. 60/372,246, filed on Apr. 12, 2002.
(60) Provisional application No. 60/350,666, filed on Nov. 13, 2001.
(60) Provisional application No. 60/343,705, filed on Nov. 8, 2001.
(60) Provisional application No. 60/310,099, filed on Aug. 3, 2001.
(60) Provisional application No. 60/302,814, filed on Jul. 3, 2001.

(51) Int. Cl. C12Q 1/68; C07H 21/04; C12P 21/02; C12N 5/06; C07K 14/47

(52) U.S. Cl. 435/6; 435/69.1; 435/320.1; 435/325; 530/350; 536/23.5

(57) ABSTRACT

Described herein are genes whose expression are up-regulated or down-regulated in bladder cancer. Also described are such genes whose expression is further up-regulated or down-regulated in drug-resistant bladder cancer cells. Related methods and compositions that can be used for diagnosis, prognosis, or treatment of bladder cancer are disclosed. Also described herein are methods that can be used to identify modulators of bladder cancer.

### CROSS-REFERENCES TO RELATED APPLICATIONS

This application is related to U.S. S No. 60/302,814, filed Jul. 3, 2001; U.S. S No. 60/310,099, filed Aug. 3, 2001; U.S. S No. 60/343,705, filed Nov. 8, 2001; U.S. S No. 60/350,666, filed Nov. 13, 2001; and U.S. S No. 60/372,246, filed Apr. 12, 2001, each of which is incorporated herein by reference.

### FIELD OF THE INVENTION

The invention relates to the identification of nucleic acid and protein expression profiles and nucleic acids, products, and antibodies thereto that are involved in bladder cancer; and to the use of such expression profiles and compositions in the diagnosis, prognosis, and therapy of bladder cancer. The invention further relates to methods for identifying and using agents and/or targets that inhibit bladder cancer.

### BACKGROUND OF THE INVENTION

In the United States, over 50,000 new cases of bladder cancer are diagnosed annually, and more than 10,000 deaths will be attributed to bladder cancer. Bladder cancer is now the fourth most common cancer among American men and the ninth most common cancer among American women. It occurs three times more frequently in men than in women, and it occurs roughly twice more frequently in white versus black men.

Bladder cancer rarely occurs in people younger than 40 years of age, being primarily a disease of older men. Nonetheless, bladder cancer is a significant cause of illness and death in the United States. The risk of bladder cancer increases steeply with age, with over half of all bladder cancer deaths occurring after age 70. In white men older than 65, the annual disease rate of bladder cancer is approximately 2 cases per 1,000 persons; this contrasts with a rate of 0.1 cases per 1,000 persons younger than 65.

Within the United States, bladder cancer rates are higher among people who reside in northern versus southern states, and is higher for people who live in urban versus rural areas. Although this difference suggests that environmental as well as genetic factors may contribute to the development and progression of the disease, other studies confirm that certain genes play a role in bladder cancer. For example, expression of the tumor suppressor gene p53 has been associated with an adverse prognosis for patients with invasive bladder cancer. A retrospective study of 243 patients treated by radical cystectomy found that the presence of nuclear p53 was an independent predictor for recurrence among patients with mid to late stage tumors. Esrig, et al (1994) N.E. J. Med. 331:1259-64.

Urinary bladder cancers represent a spectrum of diseases that can be grouped into three general categories: superficial, invasive, and metastatic. The prognosis for treatment is highly dependent on the stage at which the tumor is first diagnosed. A unique aspect of bladder cancer treatment is that repeated surgical biopsy is an integral part of routine patient management. This has permitted the conduct of molecular genetic studies of tumors from specific stages of the disease. The results of these studies suggest that bladder cancers develop and progress along at least two discrete pathways, which may account for differences in invasiveness and metastatic potential. Incorporating molecular genetic factors into the current paradigm for diagnosis and treatment will optimize the probability of cure and allow the quality of life for bladder cancer patients to be maintained.

Early detection and treatment can prevent reoccurrence and progression of the disease to an incurable stage. Thus, the identification of novel diagnostic markers and therapeutic targets will improve the current treatment of bladder cancer patients. While industry and academia have identified novel sequences, there has not been an equal effort exerted to identify the function of these novel sequences in disease states. The elucidation of a role for novel proteins and compounds in disease states for identification of diagnostic markers and therapeutic targets is essential for improving the current treatment of bladder cancer patients. Accordingly, provided herein are methods that can be used in diagnosis and prognosis of bladder cancer. Additionally, provided herein are molecular targets for therapeutic intervention in bladder cancer and other related bladder diseases. Further provided are methods that can be used to screen candidate bioactive agents for the ability to modulate bladder cancer.

### SUMMARY OF THE INVENTION

The present invention therefore provides nucleotide sequences of genes that are up- and down-regulated in bladder cancer cells. Such genes are useful for diagnostic purposes, and also as targets for screening for therapeutic compounds that modulate bladder cancer, such as hormones or antibodies. Other aspects of the invention will become apparent to the skilled artisan by the following description of the invention.

In one aspect, the present invention provides a method of detecting a bladder cancer-associated transcript in a cell from a patient, the method comprising contacting a biological sample from the patient with a polynucleotide that selectively hybridizes to a sequence at least 80% identical to a sequence as shown in Tables 1A-13.

In one embodiment, the present invention provides a method of determining the level of a bladder cancer associated transcript in a cell from a patient.

In one embodiment, the present invention provides a method of detecting a bladder cancer-associated transcript in a cell from a patient, the method comprising contacting a biological sample from the patient with a polynucleotide that selectively hybridizes to a sequence at least 80% identical to a sequence as shown in Tables 1A-13.

In one embodiment, the polynucleotide selectively hybridizes to a sequence at least 95% identical to a sequence as shown in Tables 1A-13.

In one embodiment, the biological sample is a tissue sample. In another embodiment, the biological sample comprises isolated nucleic acids, e.g., mRNA.

In one embodiment, the polynucleotide is labeled, e.g., with a fluorescent label.

In one embodiment, the polynucleotide is immobilized on a solid surface.

In one embodiment, the patient is undergoing a therapeutic regimen to treat bladder cancer. In another embodiment, the patient is suspected of having metastatic bladder cancer.

In one embodiment, the patient is a human.

In one embodiment, the bladder cancer associated transcript is mRNA.

In one embodiment, the method further comprises the step of amplifying nucleic acids before the step of contacting the biological sample with the polynucleotide.

In another aspect, the present invention provides a method of monitoring the efficacy of a therapeutic treatment of bladder cancer, the method comprising the steps of: (i) providing a biological sample from a patient undergoing the therapeutic treatment; and (ii) determining the level of a bladder cancer-associated transcript in the biological sample by contacting the biological sample with a polynucleotide that selectively hybridizes to a sequence at least 80% identical to a sequence as shown in Tables 1A-13, thereby monitoring the efficacy of the therapy. In a further embodiment, the patient has metastatic bladder cancer. In a further embodiment, the patient has a drug resistant form of bladder cancer.

In one embodiment, the method further comprises the step of: (iii) comparing the level of the bladder cancer-associated transcript to a level of the bladder cancer-associated transcript in a biological sample from the patient prior to, or earlier in, the therapeutic treatment.

Additionally, provided herein is a method of evaluating the effect of a candidate bladder cancer drug comprising administering the drug to a patient and removing a cell sample from the patient. The expression profile of the cell is then determined. This method may further comprise comparing the expression profile to an expression profile of a healthy individual. In a preferred embodiment, said expression profile includes a gene of Tables 1A-13.

In one aspect, the present invention provides an isolated nucleic acid molecule consisting of a polynucleotide sequence as shown in Tables 1A-13.

In one embodiment, an expression vector or cell comprises the isolated nucleic acid.

In one aspect, the present invention provides an isolated polypeptide which is encoded by a nucleic acid molecule having polynucleotide sequence as shown in Tables 1A-13.

In another aspect, the present invention provides an antibody that specifically binds to an isolated polypeptide which is encoded by a nucleic acid molecule having polynucleotide sequence as shown in Tables 1A-13.

In one embodiment, the antibody is conjugated to an effector component, e.g., a fluorescent label, a radioisotope or a cytotoxic chemical.

In one embodiment, the antibody is an antibody fragment. In another embodiment, the antibody is humanized.

In one aspect, the present invention provides a method of detecting a bladder cancer cell in a biological sample from a patient, the method comprising contacting the biological sample with an antibody as described herein.

In another aspect, the present invention provides a method of detecting antibodies specific to bladder cancer in a patient, the method comprising contacting a biological sample from the patient with a polypeptide encoded by a nucleic acid comprising a sequence from Tables 1A-13.

In another aspect, the present invention provides a method for identifying a compound that modulates a bladder cancer-associated polypeptide, the method comprising the steps of: (i) contacting the compound with a bladder cancer-associated polypeptide, the polypeptide encoded by a polynucleotide that selectively hybridizes to a sequence at least 80% identical to a sequence as shown in Tables 1A-13; and (ii) determining the functional effect of the compound upon the polypeptide.

In one embodiment, the functional effect is a physical effect, an enzymatic effect, or a chemical effect.

In one embodiment, the polypeptide is expressed in a eukaryotic host cell or cell membrane. In another embodiment, the polypeptide is recombinant.

In one embodiment, the functional effect is determined by measuring ligand binding to the polypeptide.

In another aspect, the present invention provides a method of inhibiting proliferation of a bladder cancer-associated cell to treat bladder cancer in a patient, the method comprising the step of administering to the subject a therapeutically effective amount of a compound identified as described herein.

In one embodiment, the compound is an antibody.

In another aspect, the present invention provides a drug screening assay comprising the steps of: (i) administering a test compound to a mammal having bladder cancer or to a cell sample isolated therefrom; (ii) comparing the level of gene expression of a polynucleotide that selectively hybridizes to a sequence at least 80% identical to a sequence as shown in Tables 1A-13 in a treated cell or mammal with the level of gene expression of the polynucleotide in a control cell sample or mammal, wherein a test compound that modulates the level of expression of the polynucleotide is a candidate for the treatment of bladder cancer.

In one embodiment, the control is a mammal with bladder cancer or a cell sample therefrom that has not been treated with the test compound. In another embodiment, the control is a normal cell or mammal.

In one embodiment, the test compound is administered in varying amounts or concentrations. In another embodiment, the test compound is administered for varying time periods. In another embodiment, the comparison can occur after addition or removal of the drug candidate.

In one embodiment, the levels of a plurality of polynucleotides that selectively hybridize to a sequence at least 80% identical to a sequence as shown in Tables 1A-13 are individually compared to their respective levels in a control cell sample or mammal. In a preferred embodiment the plurality of polynucleotides is from three to ten.

In another aspect, the present invention provides a method for treating a mammal having bladder cancer comprising administering a compound identified by the assay described herein.

In another aspect, the present invention provides a pharmaceutical composition for treating a mammal having bladder cancer, the composition comprising a compound identified by the assay described herein and a physiologically acceptable excipient.

In one aspect, the present invention provides a method of screening drug candidates by providing a cell expressing a gene that is up- and down-regulated as in a bladder cancer. In one embodiment, a gene is selected from Tables 1A-13. The method further includes adding a drug candidate to the cell and determining the effect of the drug candidate on the expression of the expression profile gene.

In one embodiment, the method of screening drug candidates includes comparing the level of expression in the absence of the drug candidate to the level of expression in the presence of the drug candidate, wherein the concentration of the drug candidate can vary when present, and wherein the comparison can occur after addition or removal of the drug candidate. In a preferred embodiment, the cell expresses at least two expression profile genes. The profile genes may show an increase or decrease.

Also provided is a method of evaluating the effect of a candidate bladder cancer drug comprising administering the drug to a transgenic animal expressing or over-expressing the bladder cancer modulatory protein, or an animal lacking the bladder cancer modulatory protein, for example as a result of a gene knockout.

Moreover, provided herein is a biochip comprising one or more nucleic acid segments of Tables 1A-13, wherein the biochip comprises fewer than 1000 nucleic acid probes. Preferably, at least two nucleic acid segments are included. More preferably, at least three nucleic acid segments are included.

Furthermore, a method of diagnosing a disorder associated with bladder cancer is provided. The method comprises determining the expression of a gene of Tables 1A-13 in a first tissue type of a first individual, and comparing the distribution to the expression of the gene from a second normal tissue type from the first individual or a second unaffected individual. A difference in the expression indicates that the first individual has a disorder associated with bladder cancer.

In a further embodiment, the biochip also includes a polynucleotide sequence of a gene that is not up- and down-regulated in bladder cancer.

In one embodiment a method for screening for a bioactive agent capable of interfering with the binding of a bladder cancer modulating protein (bladder cancer modulatory protein) or a fragment thereof and an antibody which binds to said bladder cancer modulatory protein or fragment thereof. In a preferred embodiment, the method comprises combining a bladder cancer modulatory protein or fragment thereof, a candidate bioactive agent and an antibody which binds to said bladder cancer modulatory protein or fragment thereof. The method further includes determining the binding of said bladder cancer modulatory protein or fragment thereof and said antibody. Wherein there is a change in binding, an agent is identified as an interfering agent. The interfering agent can be an agonist or an antagonist. Preferably, the agent inhibits bladder cancer.

Also provided herein are methods of eliciting an immune response in an individual. In one embodiment a method provided herein comprises administering to an individual a composition comprising a bladder cancer modulating protein, or a fragment thereof. In another embodiment, the protein is encoded by a nucleic acid selected from those of Tables 1A-13.

Further provided herein are compositions capable of eliciting an immune response in an individual. In one embodiment, a composition provided herein comprises a bladder cancer modulating protein, preferably encoded by a nucleic acid of Tables 1A-13 or a fragment thereof, and a pharmaceutically acceptable carrier. In another embodiment, said composition comprises a nucleic acid comprising a sequence encoding a bladder cancer modulating protein, preferably selected from the nucleic acids of Tables 1A-13, and a pharmaceutically acceptable carrier.

Also provided are methods of neutralizing the effect of a bladder cancer protein, or a fragment thereof, comprising contacting an agent specific for said protein with said protein in an amount sufficient to effect neutralization. In another embodiment, the protein is encoded by a nucleic acid selected from those of Tables 1A-13.

In another aspect of the invention, a method of treating an individual for bladder cancer is provided. In one embodiment, the method comprises administering to said individual an inhibitor of a bladder cancer modulating protein. In another embodiment, the method comprises administering to a patient having bladder cancer an antibody to a bladder cancer modulating protein conjugated to a therapeutic moiety. Such a therapeutic moiety can be a cytotoxic agent or a radioisotope.

### DETAILED DESCRIPTION OF THE INVENTION

In accordance with the objects outlined above, the present invention provides novel methods for diagnosis and prognosis evaluation for bladder disease (BD), e.g., cancer, including metastatic bladder cancer, as well as methods for screening for compositions which modulate bladder diseases. Also provided are methods and compositions for treating bladder disease. Various related conditions where these markers may be useful also, include, e.g., carcinoma in situ, various stages of papillary carcinomas; and such conditions in different stages, layers, structural portions, etc.

Recent advances in molecular medicine, generally, have increased the interest in tumor-specific cell surface antigens that could serve as diagnostic or prognostic markers, or as targets for various immunotherapeutic or small molecule strategies. Antigens suitable for immunotherapeutic strategies should be highly expressed in cancer tissues and ideally not expressed in other, e.g., normal, adult tissues. Expression in tissues that are dispensable for life, however, may be tolerated, as a physiological consequence of such expression would be limited. Examples of such antigens in cancers other than bladder cancer include Her2/neu and the B-cell antigen CD20. Humanized monclonal antibodies directed to Her2/neu (Herceptin®/trastuzumab) are currently in use for the treatment of metastatic breast cancer. Ross and Fletcher (1998) Stem Cells 16:413-428. Similarly, anti-CD20 monoclonal antibodies (Rituxin®/rituximab) are used to effectively treat non-Hodgkin's lymphoma. Maloney, et al. (1997) Blood 90:2188-2195; and Leget and Czuczman (1998) Curr. Opin. Oncol. 10:548-551.

Definitions

The term “bladder cancer protein” or “bladder cancer polynucleotide” or “bladder cancer-associated transcript” refers to nucleic acid and polypeptide polymorphic variants, alleles, mutants, and interspecies homologues that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to a nucleotide sequence of or associated with a gene of Tables 1A-13; (2) bind to antibodies, e.g., polyclonal antibodies, raised against an immunogen comprising an amino acid sequence encoded by a nucleotide sequence of or associated with a gene of Tables 1A-13, and conservatively modified variants thereof; (3) specifically hybridize under stringent hybridization conditions to a nucleic acid sequence, or the complement thereof of Tables 1A-13 and conservatively modified variants thereof; or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino sequence identity, preferably over a region of over a region of at least about 25, 50, 100, 200, 500, 1000, or more amino acid, to an amino acid sequence encoded by a nucleotide sequence of or associated with a gene of Tables 1A-13. A polynucleotide or polypeptide sequence is typically from a mammal including, but not limited to, primate, e.g., human; rodent, e.g., rat, mouse, hamster; cow, pig, horse, sheep, or other mammal. A “bladder cancer polypeptide” and a “bladder cancer polynucleotide,” include both naturally occurring or recombinant forms.

A “full length” bladder cancer protein or nucleic acid refers to a bladder cancer polypeptide or polynucleotide sequence, or a variant thereof, that contains all of the elements normally contained in one or more naturally occurring, wild type bladder cancer polynucleotide or polypeptide sequences. The “full length” may be prior to, or after, various stages of splicing, including alternative splicing, or post-translation processing.

“Biological sample” as used herein is a sample of biological tissue or fluid, e.g., that contains nucleic acids or polypeptides of a bladder cancer protein, polynucleotide, or transcript. Such samples include, but are not limited to, tissue isolated from primates, e.g., humans, or rodents, e.g., mice and rats. Biological samples may also include sections of tissues such as biopsy and autopsy samples, frozen sections taken for histologic purposes, blood, plasma, serum, sputum, stool, urine, tears, mucus, hair, skin, etc. Biological samples also include explants and primary and/or transformed cell cultures derived from patient tissues. A biological sample is typically obtained from a eukaryotic organism, most preferably a mammal such as a primate, e.g., chimpanzee or human; cow; dog; cat; a rodent, e.g., guinea pig, rat, or mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (e.g., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, substitutions, naturally occurring variants, e.g., polymorphic or allelic, and manmade variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison computer algorithm, test and reference sequences, subsequence coordinates, and sequence algorithm program parameters are typically designated. Default or alternative program parameters can be selected. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from about 20-600, usually about 50-200, more usually about 100-150 in which a sequence may be compared to a reference sequence of the same number of contiguous positions after the two sequences are optimally aligned. Methods of alignment of sequences for comparison are well-known in the art. Optimal alignment of sequences for comparison can be conducted, by, e.g., the local homology algorithm of Smith and Waterman (1981) Adv. Appl. Math. 2:482, the homology alignment algorithm of Needleman and Wunsch (1970) J. Mol. Biol. 48:443-453, the search for similarity method of Pearson and Lipman (1988) Proc. Nat'l Acad. Sci. USA 85:2444-448, computerized implementations of these algorithms (GAP, BESTFIT, FASTA, and TFASTA in the Wisconsin Genetics Software Package, Genetics Computer Group, 575 Science Dr., Madison, Wis.), or manual alignment and visual inspection (see, e.g., Ausubel, et al. (eds. 1995 and supplements) Current Protocols in Molecular Biology Lippincott.

Preferred algorithms suitable for determining percent sequence identity and sequence similarity include the BLAST and BLAST 2.0 algorithms. See Altschul, et al. (1977) Nuc. Acids Res. 25:3389-3402; and Altschul, et al. (1990) J. Mol. Biol. 215:403-410. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength of 3, expectation (E) of 10, and the BLOSUM62 scoring matrix (see Henikoff and Henikoff (1989) Proc. Nat'l Acad. Sci. USA 89:10915-919) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Nat'l Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments typically denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogeneous, e.g., 100% pure.

The terms “polypeptide,” “peptide,” and “protein” are used interchangeably herein to refer to a polymer of amino acid residues. The terms apply to amino acid polymers in which at least one amino acid residue is an artificial chemical mimetic of a corresponding naturally occurring amino acid, as well as to naturally occurring amino acid polymers, those containing modified residues, and a non-naturally occurring amino acid polymer.

The term “amino acid” embraces naturally occurring or synthetic amino acids, amino acid analogs, and amino acid mimetics. Naturally occurring amino acids are those encoded by the genetic code, as well as those amino acids that are later modified, e.g., hydroxyproline, γ-carboxyglutamate, and O-phosphoserine. Amino acid analogs include compounds that share a basic chemical structure with a naturally occurring amino acid, e.g., an a carbon that is bound to a hydrogen, a carboxyl group, an amino group, or an R group, e.g., homoserine, norleucine, methionine sulfoxide, methionine methyl sulfonium. Such analogs may have modified R groups (e.g., norleucine) or modified peptide backbones, but share some basic chemical structure with a naturally occurring amino acid. Amino acid mimetics include chemical compounds that have a structure that is different from the general chemical structure of an amino acid, but that function similarly to a naturally occurring amino acid.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to amino acid or nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG, and GCU all encode the amino acid alanine. Thus, at each position where an alanine is specified by a codon, the codon can be altered to another of the corresponding codons described without altering the encoded polypeptide. Such nucleic acid variations are “silent variations,” which are one species of conservatively modified variations. Each nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions, or additions to a nucleic acid, peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M). See, e.g., Creighton (1984) Proteins: Structure and Molecular Properties Freeman.

Macromolecular structures such as polypeptide structures can be described in terms of various levels of organization. See, e.g., Alberts, et al. (eds. 2001) Molecular Biology of the Cell (4th ed.) Garland; and Cantor and Schimmel (1980) Biophysical Chemistry Part I: The Conformation of Biological Macromolecules Freeman. “Primary structure” refers to the amino acid sequence of a particular peptide. “Secondary structure” refers to locally ordered, three dimensional structures within a polypeptide. These structures are commonly known as domains, which are portions of a polypeptide that often form a compact unit of the polypeptide, and are typically about 25-500 amino acids long. Typical domains are made of sections of lesser organization such as stretches of β-sheet and α-helices. “Tertiary structure” refers to the complete three dimensional structure of a polypeptide monomer. “Quaternary structure” refers to the three dimensional structure formed, usually by the non-covalent association of independent tertiary units. Anisotropic terms are also known as energy terms.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers, including longer lengths, e.g., 200, 300, 500, 1000, 2000, 3000, 5000, 7000, 10,000, etc. A nucleic acid of the present invention will generally contain phosphodiester bonds. In some cases, nucleic acid analogs are included that may have alternate backbones, e.g., phosphoramidate (Beaucage, et al. (1993) Tetrahedron 49:1925-963 and references therein; Letsinger (1970) J. Org. Chem. 35:3800-803; Sprinzl, et al. (1977) Eur. J. Biochem. 81:579-589; Letsinger, et al. (1986) Nucl. Acids Res. 14:3487-499; Sawai, et al. (1984) Chem. Lett. 805; Letsinger, et al. (1988) J. Am. Chem. Soc. 110:4470-471; and Pauwels, et al. (1986) Chemica Scripta 26:141-149); phosphorothioate (Mag, et al. (1991), Nucleic Acids Res. 19:1437-441; and U.S. Pat. No. 5,644,048); phosphorodithioate (Brill, et al. (1989) J. Am. Chem. Soc. 111:2321-322); O-methylphophoroamidite linkages (see Eckstein (1992) Oligonucleotides and Analogues: A Practical Approach Oxford Univ. Press); and peptide nucleic acid backbones and linkages (see Egholm (1992) J. Am. Chem. Soc. 114:1895-897; Meier, et al. (1992) Chem. Int. Ed. Engl. 31:1008-010; Nielsen (1993) Nature 365:566-568; Carlsson, et al. (1996) Nature 380:207. Other analog nucleic acids include those with positively charged backbones (Denpcy, et al. (1995) Proc. Nat'l Acad. Sci. USA 92:6097-101); non-ionic backbones (U.S. Pat. Nos. 5,386,023; 5,637,684; 5,602,240; 5,216,141; and 4,469,863; Kiedrowshi, et al. (1991) Angew. Chem. Intl. Ed. English 30:423-426; Letsinger, et al. (1988) J. Am. Chem. Soc. 110:4470-471; Jung, et al. (1994) Nucleoside and Nucleotide 13:1597-xxx; Chapters 2-3 of Sanghvi and Cook (eds. 1994) Carbohydrate Modifications in Antisense Research ACS Symposium Series 580; Mesmaeker, et al. (1994) Bioorganic and Medicinal Chem. Lett. 4:395-398; Jeffs, et al. (1994) J. Biomolecular NMR 34:17; Horn, et al. (1996) Tetrahedron Lett. 37:743-xxx); and non-ribose backbones (see U.S. Pat. Nos. 5,235,033 and 5,034,506, and Chapters 6-7 of Sanghvi and Cook (eds. 1994) Carbohydrate Modifications in Antisense Research ACS Symposium Series 580. Nucleic acids containing one or more carbocyclic sugars are also contemplated. See Jenkins and Turner (1995) Chem. Soc. Rev. 24:169-176. Several nucleic acid analogs are described in Rawls (page 35, Jun. 2, 1997) C&E News. Modifications of the ribose-phosphate backbone may be made, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

Particularly preferred are peptide nucleic acids (PNA) which include peptide nucleic acid analogs. These backbones are substantially non-ionic under neutral conditions, in contrast to the highly charged phosphodiester backbone of naturally occurring nucleic acids. The PNA backbone typically exhibits improved hybridization kinetics, exhibiting larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. And due to their non-ionic nature, hybridization of the polymers is relatively insensitive to salt concentration. In addition, PNAs are not as easily degraded by cellular enzymes, and can be more stable.

The nucleic acids may be single stranded or double stranded, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA, or a hybrid, where the nucleic acid may contain combinations of deoxyribo- and ribo-nucleotides, and combinations of bases, including uracil, adenine, thymine, cytosine, guanine, inosine, xanthine, hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g., the individual units of a peptide nucleic acid, each containing a base, are referred to herein as a nucleoside.

A “label” or “detectable moiety” is a composition detectable by spectroscopic, photochemical, biochemical, immunochemical, chemical, or other physical means. Direct or indirect methods are comtemplated. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into the bladder cancer nucleic acids, proteins, and antibodies. Methods are well known for conjugating the antibody to the label, including those methods described by Hunter, et al. (1962) Nature 144:945-946; David, et al. (1974) Biochemistry 13:1014-021; Pain, et al. (1981) J. Immunol. Meth. 40:219-230; and Nygren (1982) J. Histochem, and Cytochem. 30:407-412.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to a target, e.g., an antibody. The “effector” can be a variety of molecules including, e.g., detection moieties including radioactive compounds; fluorescent compounds; an enzyme or substrate; tags such as epitope tags; a toxin; activatable moieties; a chemotherapeutic agent; a lipase; an antibiotic; a radioisotope emitting “hard”, e.g., beta radiation; or an attracting moiety.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (e.g., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. Probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which streptavidin linked label may bind. By assaying for the presence or absence of the probe, one can detect the presence or absence of the select sequence or subsequence. Diagnosis or prognosis may be based at the genomic level, or at the level of RNA or protein expression.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed, or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, e.g., using in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, e.g., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in “Overview of principles of hybridization and the strategy of nucleic acid assays” in Tijssen (1993) Hybridization with Nucleic Probes (Techniques in Biochemistry and Molecular Biology; vol. 24) Elsevier. Generally, stringent conditions are selected to be about 5-10° C. lower than the thermal melting point (Tₘ) for the specific sequence at a defined ionic strength pH. The Tₘ is the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal is at least about two times background, preferably about 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32-48° C. depending on primer length. For high stringency PCR amplification, a temperature of about 62° C. is typical, although high stringency annealing temperatures can range from about 50-65° C., depending on the primer length and specificity. Typical cycle conditions for both high and low stringency amplifications include a denaturation phase of 90-95° C. for 30-120 sec, an annealing phase lasting 30-120 sec, and an extension phase of about 72° C. for 1-2 min. Protocols and guidelines for low and high stringency amplification reactions are provided, e.g., in Innis, et al. (1990) PCR Protocols, A Guide to Methods and Applications Academic Press NY.

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least about twice background. Alternative hybridization and wash conditions can be utilized to provide conditions of similar stringency. Additional guidelines for determining hybridization parameters are provided in numerous references, e.g.,. Ausubel, et al. Current Protocols in Molecular Biology Lippincott.

The phrase “functional effects” in the context of assays for testing compounds that modulate activity of a bladder cancer protein includes the determination of a parameter that is indirectly or directly under the influence of the bladder cancer protein or nucleic acid, e.g., a functional, physical, or chemical effect, such as the ability to decrease bladder cancer. It includes ligand binding activity; cell growth on soft agar; anchorage dependence; contact inhibition and density limitation of growth; cell viability, cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of bladder cancer cells. “Functional effects” include in vitro, in vivo, and ex vivo activities.

By “determining the functional effect” is meant assaying for a compound that increases or decreases a parameter that is indirectly or directly under the influence of a bladder cancer protein sequence, e.g., functional, enzymatic, physical and chemical effects. Such functional effects can be measured by many means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the bladder cancer protein; measuring binding activity or binding assays, e.g., binding to antibodies or other ligands, and measuring cellular proliferation or metabolism. Determination of the functional effect of a compound on bladder cancer can also be performed using bladder cancer assays, such as, in vitro assays, e.g., cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of bladder cancer cells. Functional effects can be evaluated by many means, e.g., microscopy for quantitative or qualitative measures of alterations in morphological features, measurement of changes in RNA or protein levels for bladder cancer-associated sequences, measurement of RNA stability, identification of downstream or reporter gene expression (CAT, luciferase, β-gal, GFP, and the like), e.g., via chemiluminescence, fluorescence, calorimetric reactions, antibody binding, inducible markers, and ligand binding assays.

“Inhibitors”, “activators”, and “modulators” of bladder cancer polynucleotide and polypeptide sequences are used to refer to activating, inhibitory, or modulating molecules or compounds identified using in vitro and in vivo assays of bladder cancer polynucleotide and polypeptide sequences. Inhibitors are compounds that, e.g., bind to, partially or totally block activity, decrease, prevent, delay activation, inactivate, desensitize, or down regulate the activity or expression of bladder cancer proteins, e.g., antagonists. Antisense nucleic acids may seem to inhibit expression and subsequent function of the protein. “Activators” are compounds that increase, open, activate, facilitate, enhance activation, sensitize, agonize, or up regulate bladder cancer protein activity. Inhibitors, activators, or modulators also include genetically modified versions of bladder cancer proteins, e.g., versions with altered activity, as well as naturally occurring and synthetic ligands, antagonists, agonists, antibodies, small chemical molecules and the like. Such assays for inhibitors and activators include, e.g., expressing the bladder cancer protein in vitro, in cells, or cell membranes, applying putative modulator compounds, and then determining the functional effects on activity, as described above. Activators and inhibitors of bladder cancer can also be identified by incubating bladder cancer cells with the test compound and determining increases or decreases in the expression of 1 or more bladder cancer proteins, e.g., 1, 2, 3, 4, 5, 10, 15, 20, 25, 30, 40, 50 or more bladder cancer proteins, such as bladder cancer proteins encoded by the sequences set out in Tables 1A-13.

Samples or assays comprising bladder cancer proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a polypeptide is achieved when the activity value relative to the control is about 80%, preferably about 50%, more preferably about 25-0%. Activation of a bladder cancer polypeptide is achieved when the activity value relative to the control (untreated with activators) is about 110%, more preferably about 150%, more preferably about 200-500% (e.g., two to five fold higher relative to the control), more preferably about 1000-3000% higher.

The phrase “changes in cell growth” refers to a change in cell growth and proliferation characteristics in vitro or in vivo, such as cell viability, formation of foci, anchorage independence, semi-solid or soft agar growth, changes in contact inhibition and density limitation of growth, loss of growth factor or serum requirements, changes in cell morphology, gaining or losing immortalization, gaining or losing tumor specific markers, ability to form or suppress tumors when injected into suitable animal hosts, and/or immortalization of the cell. See, e.g., pp. 231-241 of Freshney (1994) Culture of Animal Cells: A Manual of Basic Technique (3d ed.).

“Tumor cell” refers to precancerous, cancerous, and normal cells in a tumor.

“Cancer cells,” “transformed” cells or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. Transformation is associated with phenotypic changes, such as immortalization of cells, aberrant growth control, nonmorphological changes, and/or malignancy. See, Freshney (2000) Culture of Animal Cells: A Manual of Basic Technique (4th ed.) Wiley-Liss.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul (ed. 1999) Fundamental Immunology (4th ed.) Raven.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. See Paul (ed. 1999) Fundamental Immunology (4th ed.) Raven. While various antibody fragments are defined in terms of the digestion of an intact antibody, such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries. See, e.g., McCafferty, et al. (1990) Nature 348:552-554.

For preparation of antibodies, e.g., recombinant, monoclonal, or polyclonal antibodies, many techniques can be used. See, e.g., Kohler and Milstein (1975) Nature 256:495-497; Kozbor, et al. (1983) Immunology Today 4:72; Cole, et al. pp. 77-96 in Reisfeld and Sell (1985) Monoclonal Antibodies and Cancer Therapy Liss; Coligan (1991) Current Protocols in Immunology Lippincott; Harlow and Lane (1988) Antibodies: A Laboratory Manual CSH Press; and Goding (1986) Monoclonal Antibodies: Principles and Practice (2d ed.) Academic Press. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens. See, e.g., McCafferty, et al. (1990) Nature 348:552-554; and Marks, et al. (1992) Biotechnology 10:779-783.

A “chimeric antibody” is an antibody molecule in which (a) the constant region, or a portion thereof, is altered, replaced, or exchanged so that the antigen binding site (variable region) is linked to a constant region of a different or altered class, effector function, and/or species, or an entirely different molecule which confers new properties to the chimeric antibody, e.g., an enzyme, toxin, hormone, growth factor, drug, etc.; or (b) the variable region, or a portion thereof, is altered, replaced, or exchanged with a variable region having a different or altered antigen specificity.

Identification of Bladder Cancer-Associated Sequences

In one aspect, the expression levels of genes are determined in different patient samples for which diagnosis information is desired, to provide expression profiles. An expression profile of a particular sample is essentially a “fingerprint” of the state of the sample; while two states may have a particular gene similarly expressed, the evaluation of a number of genes simultaneously allows the generation of a gene expression profile that is characteristic of the state of the cell. That is, normal tissue (e.g., normal bladder or other tissue) may be distinguished from cancerous or metastatic cancerous tissue of the bladder, or bladder cancer tissue or metastatic bladder cancerous tissue can be compared with tissue samples of bladder and other tissues from surviving cancer patients. By comparing expression profiles of tissue in known different bladder cancer states, information regarding which genes are important (including both up- and down-regulation of genes) in each of these states is obtained.

The identification of sequences that are differentially expressed in bladder cancer versus non-bladder cancer tissue allows the use of this information in a number of ways. For example, a particular treatment regime may be evaluated: does a chemotherapeutic drug act to down-regulate bladder cancer, and thus tumor growth or recurrence, in a particular patient; or does chemotherapy or radiation therapy induce expression of particular targets. Similarly, diagnosis and treatment outcomes may be done or confirmed by comparing patient samples with the known expression profiles. Metastatic tissue can also be analyzed to determine the stage of bladder cancer in the tissue or origin of a primary tumor. Furthermore, these gene expression profiles (or individual genes) allow screening of drug candidates with an eye to mimicking or altering a particular expression profile; e.g., screening can be done for drugs that suppress the bladder cancer expression profile. This may be done by making biochips comprising sets of important bladder cancer genes, which can then be used in these screens. These methods can also be applied on the protein basis; that is, protein expression levels of the bladder cancer proteins can be evaluated for diagnostic purposes or to screen candidate agents. In addition, the bladder cancer nucleic acid sequences can be administered for gene therapy purposes, including the administration of antisense or inhibitory nucleic acids, or the bladder cancer proteins (including antibodies and other modulators thereof) administered as therapeutic drugs.

Thus the present invention provides nucleic acid and protein sequences that are differentially expressed in bladder disease or cancer relative to normal tissues and/or non-malignant bladder tissue, herein termed “bladder cancer sequences.” As outlined below, bladder cancer sequences include those that are up-regulated (e.g., expressed at a higher level) in bladder cancer, as well as those that are down-regulated (e.g., expressed at a lower level). In a preferred embodiment, the bladder cancer sequences are from humans; however, as will be appreciated by those in the art, bladder cancer sequences from other organisms may be useful in animal models of disease and drug evaluation; thus, other bladder cancer sequences are provided, from vertebrates, including mammals, including rodents (rats, mice, hamsters, guinea pigs, etc.), primates, farm animals (including sheep, goats, pigs, cows, horses, etc.) and pets, e.g., (dogs, cats, etc.). Bladder cancer sequences from other organisms may be obtained using the techniques outlined below.

Bladder cancer sequences can include both nucleic acid and amino acid sequences. Bladder cancer nucleic acid sequences are useful in a variety of applications, including diagnostic applications, which will detect naturally occurring nucleic acids, as well as screening applications. Biochips comprising nucleic acid probes or PCR microtiter plates with selected probes to the bladder cancer sequences can be generated.

A bladder cancer sequence can be initially identified by substantial nucleic acid and/or amino acid sequence homology to the bladder cancer sequences outlined herein. Such homology can be based upon the overall nucleic acid or amino acid sequence, and is generally determined as outlined below, using either homology programs or hybridization conditions.

For identifying bladder cancer-associated sequences, the bladder cancer screen typically includes comparing genes identified in different tissues, e.g., normal, non-malignant, or cancerous tissues, or tumor tissue samples from patients who have metastatic disease vs. non metastatic tissue. Other suitable tissue comparisons include comparing bladder cancer samples with metastatic cancer samples from other cancers, such as lung, bladder, gastrointestinal cancers, ovarian, etc. Samples of different stages of bladder cancer, e.g., survivor tissue, drug resistant states, and tissue undergoing metastasis, are applied to biochips comprising nucleic acid probes. The samples are first microdissected, if applicable, and treated for the preparation of mRNA. Suitable biochips are commercially available, e.g., from Affymetrix. Gene expression profiles as described herein are generated and the data analyzed.

In one embodiment, genes showing changes in expression as between normal and disease states are compared to genes expressed in other normal tissues, preferably normal bladder, but also including, and not limited to lung, heart, brain, liver, bladder, kidney, muscle, colon, small intestine, large intestine, spleen, bone, and placenta. In a preferred embodiment, those genes identified during the bladder cancer screen that are expressed in a significant amount in other tissues are removed from the profile, although in some embodiments, this is not necessary. That is, when screening for drugs, it is usually preferable that the target be disease specific, e.g., not be expressed on critical organs.

In a preferred embodiment, bladder cancer sequences are those that are up-regulated in bladder cancer; that is, the expression of these genes is higher in the bladder cancer tissue as compared to non-cancerous tissue. “Up-regulation” as used herein often means at least about a two-fold change, preferably at least about a three fold change, with at least about five-fold or higher being preferred. Unigene cluster identification numbers and accession numbers herein are for the GenBank sequence database and sequences of accession numbers are hereby expressly incorporated by reference. GenBank is known in the art, see, e.g., Benson, et al. (1998) Nuc. Acids Res. 26:1-7 and http://www.ncbi.nlm.nih.gov/. Sequences are also available in other databases, e.g., European Molecular Biology Laboratory (EMBL) and DNA Database of Japan (DDBJ).

In another preferred embodiment, bladder cancer sequences are those that are down-regulated in the bladder cancer; that is, the expression of these genes is lower in bladder cancer tissue as compared to non-cancerous tissue (see, e.g., Tables 1A-13). “Downregulation” as used herein often means at least about a two-fold change, preferably at least about a three fold change, with at least about five-fold or higher being preferred.

Informatics

The ability to identify genes that are over or under expressed in bladder cancer can additionally provide high-resolution, high-sensitivity datasets which can be used in the areas of diagnostics, therapeutics, drug development, pharmacogenetics, protein structure, biosensor development, and other related areas. For example, the expression profiles can be used in diagnostic or prognostic evaluation of patients with bladder cancer. Or as another example, subcellular toxicological information can be generated to better direct drug structure and activity correlation. See Anderson (Jun. 11-12, 1998) Pharmaceutical Proteomics: Targets, Mechanism, and Function, paper presented at the IBC Proteomics conference, Coronado, Calif. Subcellular toxicological information can also be utilized in a biological sensor device to predict the likely toxicological effect of chemical exposures and likely tolerable exposure thresholds (see U.S. Pat. No. 5,811,231). Similar advantages accrue from datasets relevant to other biomolecules and bioactive agents (e.g., nucleic acids, saccharides, lipids, drugs, and the like).

Thus, in another embodiment, the present invention provides a database that includes at least one set of assay data. The data contained in the database is acquired, e.g., using array analysis either singly or in a library format. The database can be in a form in which data can be maintained and transmitted, but is preferably an electronic database. The electronic database of the invention can be maintained on an electronic device allowing for the storage of and access to the database, such as a personal computer, but is preferably distributed on a wide area network, such as the World Wide Web.

The focus of the present section on databases that include peptide sequence data is for clarity of illustration only. It will be apparent that similar databases can be assembled for assay data acquired using an assay of the invention.

The compositions and methods for identifying and/or quantitating the relative and/or absolute abundance of a variety of molecular and macromolecular species from a biological sample undergoing bladder cancer, e.g., the identification of bladder cancer-associated sequences described herein, provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

See also Mount, et al. (2001) Bioinformatics CSH Press, NY; Durbin, et al. (eds. 1999) Biological Sequence Analysis: Probabilistic Models of Proteins and Nucleic Acids Cambridge Univ. Press; Baxevanis and Oeullette (eds. 1998) Bioinformatics: A Practical Guide to the Analysis of Genes and Proteins (2d. ed.) Wiley-Liss; Rashidi and Buehler (1999) Bioinformatics: Basic Applications in Biological Science and Medicine CRC Press; Setubal, et al. (eds 1997) Introduction to Computational Molecular Biology Brooks/Cole; Misener and Krawetz (eds. 2000) Bioinformatics: Methods and Protocols Oxford Univ. Press; Higgins and Taylor (eds. 2000) Bioinformatics: Sequence, Structure, and Databanks: A Practical Approach Oxford Univ. Press; Brown (2001) Bioinformatics: A Biologist's Guide to Biocomputing and the Internet Eaton Pub.; Han and Kamber (2000) Data Mining: Concepts and Techniques Kaufmann Pub.; and Waterman (1995) Introduction to Computational Biology: Maps, Sequences, and Genomes Chap and Hall.

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g., a neoplastic lesion or another tissue specimen to be analyzed for bladder cancer. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 1 OBaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

Characteristics of Bladder Cancer-Associated Proteins

Bladder cancer proteins of the present invention may be classified as secreted proteins, transmembrane proteins or intracellular proteins. In one embodiment, the bladder cancer protein is an intracellular protein. Intracellular proteins may be found in the cytoplasm and/or in the nucleus. Intracellular proteins are involved in all aspects of cellular function and replication (including, e.g., signaling pathways); aberrant expression of such proteins often results in unregulated or disregulated cellular processes (see, e.g., Alberts, et al. (1994) Molecular Biology of the Cell (3d ed.) Garland. For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing proteins is the presence in the proteins of one or more structural motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of amino acid sequence; thus, an analysis of the sequence of proteins may provide insight into both the enzymatic potential of the molecule and/or molecules with which the protein may associate. One useful database is Pfam (protein families), which is a large collection of multiple sequence alignments and hidden Markov models covering many common protein domains. Versions are available via the internet from Washington University in St. Louis, the Sanger Center in England, and the Karolinska Institute in Sweden. See, e.g., Bateman, et al. (2000) Nuc. Acids Res. 28:263-266; Sonnhammer, et al. (1997) Proteins 28:405-420; Bateman, et al. (1999) Nuc. Acids Res. 27:260-262; and Sonnhammer, et al. (1998) Nuc. Acids Res. 26:320-322.

In another embodiment, the bladder cancer sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 17 consecutive hydrophobic amino acids that may be followed by charged amino acids. Therefore, upon analysis of the amino acid sequence of a particular protein, the localization and number of transmembrane domains within the protein may be predicted (see, e.g., PSORT web site http://psort.nibb.acjp/). Important transmembrane protein receptors include, but are not limited to the insulin receptor, insulin-like growth factor receptor, human growth hormone receptor, glucose transporters, transferrin receptor, epidermal growth factor receptor, low density lipoprotein receptor, epidermal growth factor receptor, leptin receptor, and interleukin receptors, e.g., IL-1 receptor, IL-2 receptor, etc.

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which may be peptides, proteins, or small molecules such as adenosine and the like. For example, growth factors such as EGF, FGF, and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell, e.g., via a glycosylphosphatidylinositol (GPI) anchor, or may themselves be transmembrane proteins. Extracellular domains also associate with the extracellular matrix and contribute to the maintenance of the cell structure.

Bladder cancer proteins that are transmembrane are particularly preferred in the present invention as they are readily accessible targets for immunotherapeutics, as are described herein. In addition, as outlined below, transmembrane proteins can be also useful in imaging modalities. Antibodies may be used to label such readily accessible proteins in situ. Alternatively, antibodies can also label intracellular proteins, in which case samples are typically permeablized to provide access to intracellular proteins.

It will also be appreciated by those in the art that a transmembrane protein can be made soluble by removing transmembrane sequences, e.g., through recombinant methods. Furthermore, transmembrane proteins that have been made soluble can be made to be secreted through recombinant means by adding an appropriate signal sequence.

In another embodiment, the bladder cancer proteins are secreted proteins; the secretion of which can be either constitutive or regulated. These proteins may have a signal peptide or signal sequence that targets the molecule to the secretory pathway. Secreted proteins are involved in numerous physiological events; e.g., if circulating, they often serve to transmit signals to various other cell types. The secreted protein may function in an autocrine manner (acting on the cell that secreted the factor), a paracrine manner (acting on cells in close proximity to the cell that secreted the factor), an endocrine manner (acting on cells at a distance, e.g, secretion into the blood stream), or exocrine (secretion, e.g., through a duct or to adjacent epithelial surface as sweat glands, sebaceous glands, pancreatic ducts, lacrimal glands, mammary glands, wax producing glands of the ear, etc.). Thus secreted molecules often find use in modulating or altering numerous aspects of physiology. Bladder cancer proteins that are secreted or released proteins are particularly preferred in the present invention as they serve as good targets for diagnostic markers, e.g., for blood, plasma, serum, or urine tests. Those which are enzymes may be antibody or small molecule targets. Others may be useful as vaccine targets, e.g., via CTL mechanisms.

Use of Bladder Cancer Nucleic Acids

As described above, bladder cancer sequence is initially identified by substantial nucleic acid and/or amino acid sequence homology or linkage to the bladder cancer sequences outlined herein. Such homology can be based upon the overall nucleic acid or amino acid sequence, and is generally determined as outlined below, using either homology programs or hybridization conditions. Typically, linked sequences on a mRNA are found on the same molecule.

The bladder cancer nucleic acid sequences of the invention, e.g., the sequences in Tables 1A-13, can be fragments of larger genes, e.g., they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the bladder cancer genes can be obtained, using techniques well known in the art for cloning either longer sequences or the full length sequences; see Ausubel, et al., supra. Much can be done by informatics and many sequences can be clustered to include multiple sequences corresponding to a single gene, e.g., systems such as UniGene (see, http://www.ncbi.nlm.nih.gov/UniGene/).

Once a bladder cancer nucleic acid is identified, it can be cloned and, if necessary, its constituent parts recombined to form the entire bladder cancer nucleic acid coding regions or the entire mRNA sequence. Once isolated from its natural source, e.g., contained within a plasmid or other vector or excised therefrom as a linear nucleic acid segment, the recombinant bladder cancer nucleic acid can be further-used as a probe to identify and isolate other bladder cancer nucleic acids, e.g., extended coding regions. It can also be used as a “precursor” nucleic acid to make modified or variant bladder cancer nucleic acids and proteins.

The bladder cancer nucleic acids of the present invention are used in several ways. In a first embodiment, nucleic acid probes to the bladder cancer nucleic acids are made and attached to biochips to be used in screening and diagnostic methods, as outlined below, or for administration, e.g., for gene therapy, vaccine, and/or antisense/inhibition applications. Alternatively, the bladder cancer nucleic acids that include coding regions of bladder cancer proteins can be put into expression vectors for the expression of bladder cancer proteins, again for screening purposes or for administration to a patient.

In a preferred embodiment, nucleic acid probes to bladder cancer nucleic acids (both the nucleic acid sequences outlined in the tables and/or the complements thereof) are made. The nucleic acid probes attached to the biochip are designed to be substantially complementary to the bladder cancer nucleic acids, e.g., the target sequence (either the target sequence of the sample or to other probe sequences, e.g., in sandwich assays), such that hybridization of the target sequence and the probes of the present invention occurs. As outlined below, this complementarity need not be perfect; there may be a number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (e.g., have some sequence in common), or separate. In some cases, PCR primers may be used to amplify signal for higher sensitivity.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can typically be covalent or non-covalent. By “non-covalent binding” and grammatical equivalents herein is meant one or more of electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluoresce. See WO 00/55627.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, e.g., the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, e.g., using linkers as are known in the art; e.g., homo-or hetero-bifunctional linkers as are well known (see 1994 Pierce Chemical Company catalog, technical section on cross-linkers, pages 155-200). In addition, in some cases, additional linkers, such as alkyl groups (including substituted and heteroalkyl groups) may be used.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In another embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

Often, amplification-based assays are performed to measure the expression level of bladder cancer-associated sequences. These assays are typically performed in conjunction with reverse transcription. In such assays, a bladder cancer-associated nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of bladder cancer-associated RNA. Methods of quantitative amplification are well known to those of skill in the art. Detailed protocols for quantitative PCR are provided, e.g., in Innis, et al. (1990) PCR Protocols: A Guide to Methods and Applications Academic Press.

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification. See, e.g., literature provided by Perkin-Elmer, e.g., www2.perkin-elmer.com.

Other suitable amplification methods include, but are not limited to, ligase chain reaction (LCR) (see Wu and Wallace (1989) Genomics 4:560-569; Landegren, et al. (1988) Science 241:1077-1080; and Barringer, et al. (1990) Gene 89:117-122), transcription amplification (Kwoh, et al. (1989) Proc. Nat'l Acad. Sci. USA 86:1173-1177), self-sustained sequence replication (Guatelli, et al. (1990) Proc. Nat'l Acad. Sci. USA 87:1874-1878), dot PCR, and linker adapter PCR, etc.

Expression of Bladder Cancer Proteins from Nucleic Acids

In a preferred embodiment, bladder cancer nucleic acids, e.g., encoding bladder cancer proteins, are used to make a variety of expression vectors to express bladder cancer proteins which can then be used in screening assays, as described below. Expression vectors and recombinant DNA technology are well known to those of skill in the art (see, e.g., Ausubel, supra, and Fernandez and Hoeffler (eds. 1999) Gene Expression Systems Academic Press) and are used to express proteins. The expression vectors may be either self-replicating extrachromosomal vectors or vectors which integrate into a host genome. Generally, these expression vectors include transcriptional and translational regulatory nucleic acid operably linked to the nucleic acid encoding the bladder cancer protein. The term “control sequences” refers to DNA sequences used for the expression of an operably linked coding sequence in a particular host organism. Control sequences that are suitable for prokaryotes, e.g., include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; a ribosome binding site is operably linked to a coding sequence if it is positioned so as to facilitate translation; two sequences may be operably linked if they are physically linked on a single polynucleotide. Generally, “operably linked” means that the DNA sequences being linked are contiguous, and, in the case of a secretory leader, contiguous and in reading phase. However, enhancers do not have to be contiguous. Linking is typically accomplished by ligation at convenient restriction sites. If such sites do not exist, synthetic oligonucleotide adaptors or linkers are used in accordance with conventional practice. Transcriptional and translational regulatory nucleic acid will generally be appropriate to the host cell used to express the bladder cancer protein. Numerous types of appropriate expression vectors, and suitable regulatory sequences are known in the art for a variety of host cells.

In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are useful in the present invention.

In addition, an expression vector may comprise additional elements. The expression vector may have two replication systems, thus allowing it to be maintained in two organisms, e.g., in mammalian or insect cells for expression and in a procaryotic host for cloning and replication. For integrating expression vectors, the expression vector may contain at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known (e.g., Fernandez and Hoeffler, supra).

In addition, in a preferred embodiment, the expression vector contains a selectable marker gene to allow the selection of transformed host cells. Selection genes are well known in the art and will vary with the host cell used.

The bladder cancer proteins of the present invention may be produced by culturing a host cell transformed with an expression vector under the appropriate conditions to induce or cause expression of the bladder cancer protein. Conditions appropriate for bladder cancer protein expression will vary with the choice of the expression vector and the host cell, and will be easily ascertained by one skilled in the art through routine experimentation or optimization. For example, the use of constitutive promoters in the expression vector will typically require optimizing the growth and proliferation of the host cell, while the use of an inducible promoter typically requires identifying the appropriate growth conditions for induction. In addition, in some embodiments, the timing of the harvest is important. For example, the baculoviral systems used in insect cell expression are lytic viruses, and harvest time selection can be crucial for product yield.

Appropriate host cells include yeast, bacteria, archaebacteria, fungi, insect, and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HUVEC (human umbilical vein endothelial cells), THPI cells (a macrophage cell line) and various other human cells and cell lines.

In a preferred embodiment, the bladder cancer proteins are expressed in mammalian cells. Mammalian expression systems include retroviral and adenoviral systems. Retroviral vector systems are described in PCT/US97/01019 and PCT/US97/01048. Of particular use are promoters from mammalian viral genes, since viral genes are often highly expressed and have a broad host range. Examples include the SV40 early promoter, mouse mammary tumor virus LTR promoter, adenovirus major late promoter, herpes simplex virus promoter, and the CMV promoter (see, e.g., Fernandez and Hoeffler, supra). Typically, transcription termination and polyadenylation sequences recognized by mammalian cells are regulatory regions located 3′to the translation stop codon and thus, together with the promoter elements, flank the coding sequence. Examples of transcription terminator and polyadenlyation signals include those derived from SV40.

Methods of introducing exogenous nucleic acid into mammalian and other hosts are well known, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In another embodiment, bladder cancer proteins are expressed in bacterial systems. Promoters from bacteriophage may also be used. Synthetic promoters and hybrid promoters are also useful; e.g., the tac promoter is a hybrid of the trp and lac promoter sequences. A bacterial promoter can include naturally occurring promoters of non-bacterial origin that have the ability to bind bacterial RNA polymerase and initiate transcription. Often an efficient ribosome binding site is desirable. The expression vector may include a signal peptide sequence that provides for secretion of the bladder cancer protein. The protein is either secreted into the growth media (gram-positive bacteria) or into the periplasmic space, located between the inner and outer membrane of the cell (gram-negative bacteria). The bacterial expression vector may include a selectable marker gene to allow for the selection of bacterial strains that have been transformed. Suitable selection genes include genes which render the bacteria resistant to drugs, e.g., ampicillin, chloramphenicol, erythromycin, kanamycin, neomycin, and tetracycline, or biosynthetic genes, e.g., those in the histidine, tryptophan, and leucine biosynthetic pathways. These components are assembled into expression vectors. Expression vectors for bacteria include vectors for Bacillus subtilis, E. coli, Streptococcus cremoris, and Streptococcus lividans, among others (e.g., Fernandez and Hoeffler, supra). The bacterial expression vectors are transformed into bacterial host cells using, e.g., calcium chloride treatment, electroporation, and other methods.

Bladder cancer proteins can also be produced in insect cells. See, e.g., Miller, et al. (1997) Baculovirus Expression Vectors: A Laboratory Manual Oxford Books; ISBN: 0716770172; and Makrides (1999) Prot. Expr. Purif. 17:183-202.

Bladder cancer protein may be produced in yeast cells. Yeast expression systems exist with expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. See, e.g., Jones, et al. (eds. 1993) The Molecular and Cellular Biology of the Yeast Saccharomyces: Gene Expression CSH Press; ISBN: 0879693657.

The bladder cancer protein may also be made as a fusion protein, using techniques well known in the art. Thus, e.g., for the creation of monoclonal antibodies, if the desired epitope is small, the bladder cancer protein may be fused to a carrier protein to form an immunogen. Alternatively, the bladder cancer protein may be made as a fusion protein to increase expression, or for other reasons. For example, when the bladder cancer protein is a bladder cancer peptide, the nucleic acid encoding the peptide may be linked to other nucleic acid for expression or purification purposes.

The bladder cancer protein is typically purified or isolated after expression. Bladder cancer proteins may be isolated or purified in a variety of ways, depending on what other components are present in the sample. Standard purification methods include electrophoretic, molecular, immunological, and chromatographic techniques, including ion exchange, hydrophobic, affinity, reverse-phase HPLC chromatography, and chromatofocusing. The bladder cancer protein may be purified using a standard anti-bladder cancer protein antibody affinity column. Ultrafiltration and diafiltration techniques, in conjunction with protein concentration, are also useful. For general guidance in suitable purification techniques, see, e.g., Scopes (1982) Protein Purification Springer-Verlag. The degree of purification necessary will vary depending on the use of the bladder cancer protein. In some instances no purification will be necessary, which may depend on the intended use.

Once expressed and purified, if necessary, the bladder cancer proteins and nucleic acids are useful in a number of applications. They may be used as immunoselection reagents, as vaccine reagents, as screening agents, etc.

Variants of Bladder Cancer Proteins

In one embodiment, the bladder cancer proteins are derivative or variant bladder cancer proteins as compared to the wild-type sequence. That is, as outlined more fully below, the derivative bladder cancer peptide will often contain at least one amino acid substitution, deletion, or insertion, with amino acid substitutions being particularly preferred. The amino acid substitution, insertion, or deletion may occur at most residues within the bladder cancer peptide.

Certain embodiments of bladder cancer proteins of the present invention are amino acid sequence variants. These variants typically fall into one or more of three classes: substitutional, insertional, or deletional variants. These variants ordinarily are prepared by site specific mutagenesis of nucleotides in the DNA encoding the bladder cancer protein, using cassette or PCR, mutagenesis, or other techniques well known in the art, to produce DNA encoding the variant, and thereafter expressing the DNA in recombinant cell culture as outlined above. However, variant bladder cancer protein fragments having up to about 100-150 residues may be prepared by in vitro synthesis using established techniques. Amino acid sequence variants are often characterized by the predetermined nature of the variation, a feature that sets them apart from naturally occurring allelic or interspecies variation of the bladder cancer protein amino acid sequence. The variants typically exhibit the same qualitative biological activity as the naturally occurring analogue, although variants can also be selected which have modified characteristics as will be more fully outlined below.

While the site or region for introducing an amino acid sequence variation is often predetermined, the mutation per se need not be predetermined. To optimize the performance of a mutation at a given site, random mutagenesis may be conducted at the target codon or region and the expressed bladder cancer variants screened for the optimal combination of desired activities. Techniques for making substitution mutations at predetermined sites in DNA having a known sequence are well known, e.g., M13 primer mutagenesis and PCR mutagenesis. Screening of the mutants is performed using assays of bladder cancer protein activities.

Amino acid substitutions are typically of single residues; insertions usually will be on the order of from about 1 to 20 amino acids, although considerably larger insertions may be tolerated. Deletions range from about 1-20 residues, although in some cases deletions may be much larger.

Substitutions, deletions, insertions, or combinations thereof may be used to arrive at a final derivative. Generally these changes are done on a few amino acids to minimize the alteration of the molecule. However, larger changes may be tolerated in certain circumstances. When small alterations in the characteristics of the bladder cancer protein are desired, substitutions are generally made in accordance with the amino acid substitution relationships provided in the definition section.

The variants typically exhibit the same qualitative biological activity and elicit the same immune response as the naturally-occurring analog, although variants also are selected to modify the characteristics of the bladder cancer proteins as needed. Alternatively, the variant may be designed such that the biological activity of the bladder cancer protein is altered. For example, glycosylation sites may be altered or removed.

Substantial changes in function or immunological identity are made by selecting substitutions that are less conservative than those described above. Substitutions may be made which more significantly affect: the structure of the polypeptide backbone in the area of the alteration, e.g., the alpha-helical or beta-sheet structure; the charge or hydrophobicity of the molecule at the target site; or the bulk of the side chain. Substitutions which are expected to produce the greatest changes in the polypeptide's properties are those in which (a) a hydrophilic residue, e.g., serine or threonine is substituted for (or by) a hydrophobic residue, e.g., leucine, isoleucine, phenylalanine, valine, or alanine; (b) a cysteine or proline is substituted for (or by) another residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine.

Covalent modifications of bladder cancer polypeptides are included within the scope of this invention. One type of covalent modification includes reacting targeted amino acid residues of a bladder cancer polypeptide with an organic derivatizing agent that is capable of reacting with selected side chains or the N-or C-terminal residues of a bladder cancer polypeptide. Derivatization with bifunctional agents is useful, e.g., for crosslinking bladder cancer polypeptides to a water-insoluble support matrix or surface for use in the method for purifying anti-bladder cancer polypeptide antibodies or screening assays. Commonly used crosslinking agents include, e.g., 1,1-bis(diazoacetyl)-2-phenylethane, glutaraldehyde, Nhydroxysuccinimide esters, e.g., esters with 4-azidosalicylic acid, homobifunctional imidoesters, including disuccinimidyl esters such as 3,3′-dithiobis(succinimidylpropionate), bifunctional malcimides such as bis-N-malcimido-1,8-octane and agents such as methyl-3-((p-azidophenyl)dithio)propioimidate.

Other modifications include deamidation of glutaminyl and asparaginyl residues to the corresponding glutamic and aspartic residues, respectively, hydroxylation of proline and lysine, phosphorylation of hydroxyl groups of serine, threonine or tyrosine residues, methylation of the amino groups of the lysine, arginine, and histidine side chains (pp. 79-86, Creighton (1984) Proteins: Structure and Molecular Properties Freeman), acetylation of the N-terminal amine, and amidation of a C-terminal carboxyl group.

Another type of covalent modification of the bladder cancer polypeptide included within the scope of this invention comprises altering the native glycosylation pattern of the polypeptide. “Altering the native glycosylation pattern” is intended for purposes herein to mean deleting one or more carbohydrate moieties found in native sequence bladder cancer polypeptide, and/or adding one or more glycosylation sites that are not present in the native sequence bladder cancer polypeptide. Glycosylation patterns can be altered in many ways. For example the use of different cell types to express bladder cancer-associated sequences can result in different glycosylation patterns.

Addition of glycosylation sites to bladder cancer polypeptides may also be accomplished by altering the amino acid sequence thereof. The alteration may be made, e.g., by the addition of, or substitution by, one or more serine or threonine residues to the native sequence bladder cancer polypeptide (for O-linked glycosylation sites). The bladder cancer amino acid sequence may optionally be altered through changes at the DNA level, particularly by mutating the DNA encoding the bladder cancer polypeptide at preselected bases such that codons are generated that will translate into the desired amino acids.

Another means of increasing the number of carbohydrate moieties on the bladder cancer polypeptide is by chemical or enzymatic coupling of glycosides to the polypeptide. Such methods are described in the art, e.g., in WO 87/05330, and pp. 259-306 in Aplin and Wriston (1981) CRC Crit. Rev. Biochem.

Removal of carbohydrate moieties present on the bladder cancer polypeptide may be accomplished chemically or enzymatically or by mutational substitution of codons encoding amino acid residues that serve as targets for glycosylation. Chemical deglycosylation techniques are known in the art. See, e.g., Hakimuddin, et al. (1987) Arch. Biochem. Biophys. 259:52-57; and Edge, et al. (1981) Anal. Biochem. 118:131-137. Enzymatic cleavage of carbohydrate moieties on polypeptides can be achieved by the use of a variety of endo-and exo-glycosidases. See, e.g., Thotakura, et al. (1987) Meth. Enzymol. 138:350-359.

Another type of covalent modification of bladder cancer comprises linking the bladder cancer polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337.

Bladder cancer polypeptides of the present invention may also be modified to form chimeric molecules comprising a bladder cancer polypeptide fused to a heterologous polypeptide or amino acid sequence. In one embodiment, a chimeric molecule comprises a fusion of a bladder cancer polypeptide with an epitope tag. The epitope tag is generally placed at the amino-or carboxyl-terminus of the bladder cancer polypeptide. The presence of such epitope-tagged forms of a bladder cancer polypeptide can be detected using an antibody against the tag polypeptide. Also, provision of the epitope tag enables the bladder cancer polypeptide to be readily purified by affinity purification using an anti-tag antibody or another type of affinity matrix that binds to the epitope tag. In an alternative embodiment, the chimeric molecule may comprise a fusion of a bladder cancer polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; HIS6 and metal chelation tags, the flu HA tag polypeptide and its antibody 12CA5 (Field, et al. (1988) Mol. Cell. Biol. 8:2159-2165); the c-myc tag and the 8F9, 3C7, 6E10, G4, B7, and 9E10 antibodies thereto (Evan, et al. (1985) Molecular and Cellular Biology 5:3610-3616); and the Herpes Simplex virus glycoprotein D (gD) tag and its antibody (Paborsky, et al. (1990) Protein Engineering 3:547-553). Other tag polypeptides include the Flag-peptide (Hopp, et al. (1988) BioTechnology 6:1204-1210); the KT3 epitope peptide (Martin, et al. (1992) Science 255:192-194); tubulin epitope peptide (Skinner, et al. (1991) J. Biol. Chem. 266:15163-15166); and the T7 gene 10 protein peptide tag (Lutz-Freyermuth, et al. (1990) Proc. Nat'l Acad. Sci. USA 87:6393-6397).

Also included are other bladder cancer proteins of the bladder cancer family, and bladder cancer proteins from other organisms, which are cloned and expressed as outlined below. Thus, probe or degenerate polymerase chain reaction (PCR) primer sequences may be used to find other related bladder cancer proteins from humans or other organisms. As will be appreciated by those in the art, particularly useful probe and/or PCR primer sequences include the unique areas of the bladder cancer nucleic acid sequence. Preferred PCR primers are from about 15-35 nucleotides in length, with from about 20-30 being preferred, and may contain inosine as needed. The conditions for the PCR reaction are well known. See, e.g., Innis (1990) PCR Protocols, supra.

Antibodies to Bladder Cancer Proteins

In a preferred embodiment, when the bladder cancer protein is to be used to generate antibodies, e.g., for immunotherapy or immunodiagnosis, the bladder cancer protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller bladder cancer protein will be able to bind to the full-length protein, particularly linear epitopes. In a preferred embodiment, the epitope is unique; that is, antibodies generated to a unique epitope show little or no cross-reactivity.

Methods of preparing polyclonal antibodies are known (see, e.g., Coligan, supra; and Harlow and Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the tables or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected as appropriate.

The antibodies may be monoclonal antibodies. Monoclonal antibodies may be prepared using hybridoma methods, such as those described by Kohler and Milstein (1975) Nature 256:495-497. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1A-13 or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (pp. 59-103, Goding (1986) Monoclonal Antibodies: Principles and Practice Academic Press). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are typically monoclonal, preferably human or humanized, antibodies that have binding specificities for at least two different antigens or that have binding specificities for two epitopes on the same antigen. In one embodiment, one of the binding specificities is for a protein encoded by a nucleic acid Tables 1A-13 or a fragment thereof, the other one is for another antigen, and preferably for a cell-surface protein or receptor or receptor subunit, preferably one that is tumor specific. Alternatively, tetramer-type technology may create multivalent reagents.

In a preferred embodiment, the antibodies to bladder cancer protein are capable of reducing or eliminating a biological function of a bladder cancer protein, as is described below. That is, the addition of anti-bladder cancer protein antibodies (either polyclonal or preferably monoclonal) to bladder cancer tissue (or cells containing bladder cancer) may reduce or eliminate the bladder cancer. Generally, at least about 25% decrease in activity, growth, size, or the like is preferred, with at least about 50% being particularly preferred, and about a 95-100% decrease being especially preferred.

In a preferred embodiment the antibodies to the bladder cancer proteins are humanized antibodies (e.g., Xenerex Biosciences; Medarex, Inc.; Abgenix, Inc.; Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat, or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fe), typically that of a human immunoglobulin. See Jones, et al. (1986) Nature 321:522-525; Riechmann, et al. (1988) Nature 332:323-329; and Presta (1992) Curr. Op. Struct. Biol. 2:593-596. Humanization can be performed, e.g., following the method of Winter and co-workers (see Jones, et al. (1986) Nature 321:522-525; Riechmann, et al. (1988) Nature 332:323-327; Verhoeyen, et al. (1988) Science 239:1534-1536), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

Human antibodies can also be produced using various techniques known in the art, including phage display libraries (Hoogenboom and Winter (1991) J. Mol. Biol. 227:381-388; Marks, et al. (1991) J. Mol. Biol. 222:581-597) or of human monoclonal antibodies (e.g., p. 77, Cole, et al. in Reisfeld and Sell (1985) Monoclonal Antibodies and Cancer Therapy Liss; and Boemer, et al. (1991) J. Imnmunol. 147:86-95). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and Marks, et al. (1992) Bio/Technology 10:779-783; Lonberg, et al. (1994) Nature 368:856-859; Morrison (1994) Nature 368:812-13; Neuberger (1996) Nature Biotechnology 14:826 commenting on Fishwild, et al. (1996) Nature Biotechnology 14:845-51; and Lonberg and Huszar (1995) Intern. Rev. Immunol. 13:65-93.

By immunotherapy is meant treatment of bladder cancer with an antibody raised against bladder cancer proteins. As used herein, immunotherapy can be passive or active. Passive immunotherapy as defined herein is the passive transfer of antibody to a recipient (patient), which may be used to target a label or toxin. Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen, leading to an immune response.

In a preferred embodiment the bladder cancer proteins against which antibodies are raised are secreted proteins as described above. Without being bound by theory, antibodies used for treatment, bind and prevent the secreted protein from binding to its receptor, thereby inactivating the secreted bladder cancer protein.

In another preferred embodiment, the bladder cancer protein to which antibodies are raised is a transmembrane protein. Without being bound by theory, antibodies used for treatment, bind the extracellular domain of the bladder cancer protein and prevent it from binding to other proteins, such as circulating ligands or cell-associated molecules. The antibody may cause down-regulation of the transmembrane bladder cancer protein. As will be appreciated by one of ordinary skill in the art, the antibody may be a competitive, noncompetitive or uncompetitive inhibitor of protein binding to the extracellular domain of the bladder cancer protein. The antibody is also an antagonist of the bladder cancer protein. Further, the antibody prevents activation of the transmembrane bladder cancer protein. In one aspect, when the antibody prevents the binding of other molecules to the bladder cancer protein, the antibody prevents growth of the cell. The antibody may also be used to target or sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity or antigen-dependent cytotoxicity (ADCC). Thus, bladder cancer is treated by administering to a patient antibodies directed against the transmembrane bladder cancer protein. Antibody-labeling may activate a co-toxin, localize a toxin payload, or otherwise provide means to locally ablate cells.

In another preferred embodiment, the antibody is conjugated to an effector moiety. The effector moiety can be a number of molecules, including labelling moieties such as radioactive labels or fluorescent labels, or can be a therapeutic moiety. In one aspect the therapeutic moiety is a smallmolecule that modulates the activity of the bladder cancer protein. In another aspect the therapeutic moiety modulates the activity of molecules associated with or in close proximity to the bladder cancer protein. The therapeutic moiety may inhibit enzymatic activity such as protease or collagenase or protein kinase activity associated with bladder cancer.

In a preferred embodiment, the therapeutic moiety can also be a cytotoxic agent. In this method, targeting the cytotoxic agent to bladder cancer tissue or cells, results in a reduction in the number of afflicted cells, thereby reducing symptoms associated with bladder cancer. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, and the like. Cytotoxic agents also include radiochemicals made by conjugating radioisotopes to antibodies raised against bladder cancer proteins, or binding of a radionuclide to a chelating agent that has been covalently attached to the antibody. Targeting the therapeutic moiety to transmembrane bladder cancer proteins not only serves to increase the local concentration of therapeutic moiety in the bladder cancer afflicted area, but also serves to reduce deleterious side effects that may be associated with the therapeutic moiety.

In another preferred embodiment, the bladder cancer protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the bladder cancer protein can be targeted within a cell, e.g., the nucleus, an antibody thereto contains a signal for that target localization, e.g., a nuclear localization signal.

The bladder cancer antibodies of the invention specifically bind to bladder cancer proteins. By “specifically bind” herein is meant that the antibodies bind to the protein with a K_{d} of at least about 0.1 mM, more usually at least about 1 μM, preferably at least about 0.1 μM or better, and most preferably, 0.01 μM or better. Selectivity of binding is also important.

Detection of Bladder Cancer Sequence for Diagnostic and Therapeutic Applications

In one aspect, the RNA expression levels of genes are determined for different cellular states in the bladder cancer phenotype. Expression levels of genes in normal tissue (e.g., not experiencing bladder cancer) and in bladder cancer tissue (and in some cases, for varying severities of bladder cancer that relate to prognosis, as outlined below), or in non-malignant disease, are evaluated to provide expression profiles. An expression profile of a particular cell state or point of development is essentially a “fingerprint” of the state. While two states may have a particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis may be performed or confirmed to determine whether a tissue sample has the gene expression profile of normal or cancerous tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus bladder cancer tissue. Genes may be turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; e.g., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays. See Lockhart (1996) Nature Biotechnology 14:1675-1680. Other techniques include, but are not limited to, quantitative reverse transcriptase PCR, northern analysis and RNase protection. As outlined above, preferably the change in expression (e.g., upregulation or downregulation) is at least about 50%, more preferably at least about 100%, more preferably at least about 150%, more preferably at least about 200%, with from about 300-1000% being especially preferred.

Evaluation may be at the gene transcript, or the protein level. The amount of gene expression may be monitored using nucleic acid probes to the DNA or RNA equivalent of the gene transcript, and the quantification of gene expression levels, or, alternatively, the final gene product itself (protein) can be monitored, e.g., with antibodies to the bladder cancer protein and standard immunoassays (ELISAs, etc.) or other techniques, including mass spectroscopy assays, 2D gel electrophoresis assays, etc. Proteins corresponding to bladder cancer genes, e.g., those identified as being important in a bladder cancer or disease phenotype, can be evaluated in a bladder cancer diagnostic test.

In a preferred embodiment, gene expression monitoring is performed simultaneously on a number of genes. Multiple protein expression monitoring can be performed as well. Similarly, these assays may be performed on an individual basis as well.

In this embodiment, the bladder cancer nucleic acid probes are attached to biochips as outlined herein for the detection and quantification of bladder cancer sequences in a particular cell. The assays are further described below in the example. PCR techniques can be used to provide greater sensitivity.

In a preferred embodiment nucleic acids encoding the bladder cancer protein are detected. Although DNA or RNA encoding the bladder cancer protein may be detected, of particular interest are methods wherein an mRNA encoding a bladder cancer protein is detected. Probes to detect mRNA can be a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example, a digoxygenin labeled riboprobe (RNA probe) that is complementary to the mRNA encoding a bladder cancer protein is detected by binding the digoxygenin with an anti-digoxygenin secondary antibody and developed with nitro blue tetrazolium and 5-bromo-4-chloro-3-indoyl phosphate.

In a preferred embodiment, various proteins from the three classes of proteins as described herein (secreted, transmembrane or intracellular proteins) are used in diagnostic assays. The bladder cancer proteins, antibodies, nucleic acids, modified proteins and cells containing bladder cancer sequences are used in diagnostic assays. This can be performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, bladder cancer proteins, including intracellular, transmembrane or secreted proteins, find use as diagnostic or prognostic markers of bladder cancer, or to assist in selecting therpay based on expression profile and archival data. Detection of these proteins in putative bladder cancer tissue allows for detection or diagnosis of bladder cancer. In one embodiment, antibodies are used to detect bladder cancer proteins. A preferred method separates proteins from a sample by electrophoresis on a gel (typically a denaturing and reducing protein gel, but may be another type of gel, including isoelectric focusing gels and the like). Following separation of proteins, the bladder cancer protein is detected, e.g., by immunoblotting with antibodies raised against the bladder cancer protein. Methods of immunoblotting are well known to those of ordinary skill in the art.

In another preferred method, antibodies to the bladder cancer protein find use in in situ imaging techniques, e.g., in histology (e.g., Asai (ed. 1993) “Antibodies in Cell Biology” Methods in Cell Biology (vol. 37). In this method cells are contacted with from one to many antibodies to the bladder cancer protein(s). Following washing to remove non-specific antibody binding, the presence of the antibody or antibodies is detected. In one embodiment the antibody is detected by incubating with a secondary antibody that contains a detectable label. In another method the primary antibody to the bladder cancer protein(s) contains a detectable label, e.g. an enzyme marker that can act on a substrate. In another preferred embodiment each one of multiple primary antibodies contains a distinct and detectable label. This method finds particular use in simultaneous screening for a plurality of bladder cancer proteins. As will be appreciated by one of ordinary skill in the art, many other histological imaging techniques are also provided by the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing bladder cancer from blood, serum, plasma, stool, urine, and other samples. Such samples, therefore, are useful as samples to be probed or tested for the presence of bladder cancer proteins. Antibodies can be used to detect a bladder cancer protein by previously described immunoassay techniques including ELISA, immunoblotting (western blotting), immunoprecipitation, BIACORE technology and the like. Conversely, the presence of antibodies may indicate an immune response against an endogenous bladder cancer protein.

In a preferred embodiment, in situ hybridization of labeled bladder cancer nucleic acid probes to tissue arrays is done. For example, arrays of tissue samples, including bladder cancer tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

In a preferred embodiment, the bladder cancer proteins, antibodies, nucleic acids, modified proteins and cells containing bladder cancer sequences are used in prognosis assays. As above, gene expression profiles can be generated that correlate to bladder cancer, clinical, pathological, or other information, e.g., in terms of long term prognosis. Again, this may be done on either a protein or gene level, with the use of genes being preferred. Single or multiple genes may be useful in various combinations. As above, bladder cancer probes may be attached to biochips for the detection and quantification of bladder cancer sequences in a tissue or patient. The assays proceed as outlined above for diagnosis. PCR methods may provide more sensitive and accurate quantification.

Assays for Therapeutic Compounds

In a preferred embodiment members of the proteins, nucleic acids, and antibodies as described herein are used in drug screening assays. The bladder cancer proteins, antibodies, nucleic acids, modified proteins and cells containing bladder cancer sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent. See, e.g., Zlokarnik, et al. (1998) Science 279:84-88; and Heid (1996) Genome Res. 6:986-94.

In a preferred embodiment, the bladder cancer proteins, antibodies, nucleic acids, modified proteins and cells containing the native or modified bladder cancer proteins are used in screening assays. That is, the present invention provides novel methods for screening for compositions which modulate the bladder cancer phenotype or an identified physiological function of a bladder cancer protein. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays may be executed. In a preferred embodiment, assays may be run on an individual gene or protein level. That is, having identified a particular gene as up regulated in bladder cancer, test compounds can be screened for the ability to modulate gene expression or for binding to the bladder cancer protein. “Modulation” thus includes both an increase and a decrease in gene expression. The preferred amount of modulation will depend on the original change of the gene expression in normal versus tissue undergoing bladder cancer, with changes of at least about 10%, preferably about 50%, more preferably about 100-300%, and in some embodiments about 300-1000% or greater. Thus, if a gene exhibits about 4-fold increase in bladder cancer tissue compared to normal tissue, a decrease of about four-fold is often desired; similarly, about 10-fold decrease in bladder cancer tissue compared to normal tissue often provides a target value of about 10-fold increase in expression to be induced by the test compound.

The amount of gene expression may be monitored using nucleic acid probes and the quantification of gene expression levels, or, alternatively, the gene product itself can be monitored, e.g., through the use of antibodies to the bladder cancer protein and standard immunoassays. Proteomics and separation techniques may also allow quantification of expression.

In a preferred embodiment, gene expression or protein monitoring of a number of entities, e.g., an expression profile, is monitored simultaneously. Such profiles will typically involve a plurality of those entities described herein.

In this embodiment, the bladder cancer nucleic acid probes are attached to biochips as outlined herein for the detection and quantification of bladder cancer sequences in a particular cell. Alternatively, PCR may be used. Thus, a series, e.g., of microtiter plate, may be used with dispensed primers in desired wells. A PCR reaction can then be performed and analyzed for each well.

Expression monitoring can be performed to identify compounds that modify the expression of one or more bladder cancer-associated sequences, e.g., a polynucleotide sequence set out in Tables 1A-13. Generally, in a preferred embodiment, a test modulator is added to the cells prior to analysis. Moreover, screens are also provided to identify agents that modulate bladder cancer, modulate bladder cancer proteins, bind to a bladder cancer protein, or interfere with the binding of a bladder cancer protein and an antibody or other binding partner.

The term “test compound” or “drug candidate” or “modulator” or grammatical equivalents as used herein describes a molecule, e.g., protein, oligopeptide, small organic molecule, polysaccharide, polynucleotide, etc., to be tested for the capacity to directly or indirectly alter the bladder cancer phenotype or the expression of a bladder cancer sequence, e.g., a nucleic acid or protein sequence. In preferred embodiments, modulators alter expression profiles, or expression profile nucleic acids or proteins provided herein. In one embodiment, the modulator suppresses a bladder cancer phenotype, e.g., to a normal tissue or non-malignant fingerprint. In another embodiment, a modulator induced a bladder cancer phenotype. Generally, a plurality of assay mixtures are run in parallel with different agent concentrations to obtain a differential response to the various concentrations. Typically, one of these concentrations serves as a negative control, e.g., at zero concentration or below the level of detection.

Drug candidates encompass numerous chemical classes, though typically they are organic molecules, preferably small organic compounds having a molecular weight of more than about 100 and less than about 2,500 daltons. Preferred small molecules are less than about 2000, or less than about 1500 or less than about 1000 or less than about 500 D. Candidate agents comprise functional groups necessary for structural interaction with proteins, particularly hydrogen bonding, and typically include at least an amine, carbonyl, hydroxyl, or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

In one aspect, a modulator will neutralize the effect of a bladder cancer protein. By “ineutralize” is meant that activity of a protein is inhibited or blocked and the consequent effect on the cell.

In certain embodiments, combinatorial libraries of potential modulators will be screened for an ability to bind to a bladder cancer polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (e.g., the number of amino acids in a polypeptide compound). Millions of chemical compounds can be synthesized through such combinatorial mixing of chemical building blocks. See, e.g., Gallop, et al. (1994) J. Med. Chem. 37:1233-1251.

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Pept. Prot. Res. 37:487-493, Houghton, et al. (1991) Nature 354:84-88); peptoids (PCT Publication No WO 91/19735); encoded peptides (PCT Publication WO 93/20242); random bio-oligomers (PCT Publication WO 92/00091); benzodiazepines (U.S. Pat. No. 5,288,514); diversomers such as hydantoins, benzodiazepines, and dipeptides (Hobbs, et al. (1993) Proc. Nat'l Acad. Sci. USA 90:6909-6913); vinylogous polypeptides (Hagihara, et al. (1992) J. Amer. Chem. Soc. 114:6568-6570); nonpeptidal peptidomimetics with a Beta-D-Glucose scaffolding (Hirschmann, et al. (1992) J. Amer. Chem. Soc. 114:9217-9218); analogous organic syntheses of small compound libraries (Chen, et al. (1994) J. Amer. Chem. Soc. 116:2661-2662); oligocarbamates (Cho, et al. (1993) Science 261:1303-1305); and/or peptidyl phosphonates (Campbell, et al. (1994) J. Org. Chem. 59:658-xxx). See, generally, Gordon, et al. (1994) J. Med. Chem. 37:1385-1401, nucleic acid libraries (see, e.g., Strategene, Corp.); peptide nucleic acid libraries (see, e.g., U.S. Pat. No. 5,539,083); antibody libraries (see, e.g., Vaughn, et al. (1996) Nature Biotechnology 14:309-314, and PCT/US96/10287); carbohydrate libraries (Liang, et al. (1996) Science 274:1520-1522, and U.S. Pat. No. 5,593,853); and small organic molecule libraries (see, e.g., benzodiazepines, Baum (p. 33, Jan. 18, 1993) C&E News); isoprenoid (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available. See, e.g., 357 MPS, 390 MPS, Advanced Chem Tech, Louisville Ky.; Symphony, Rainin, Woburn, Mass.; 433A Applied Biosystems, Foster City, Calif.; and 9050 Plus, Millipore, Bedford, Mass.

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J.; Asinex, Moscow, Ru; Tripos, Inc., St. Louis, Mo.; ChemStar, Ltd, Moscow, RU; 3D Pharmaceuticals, Exton, Pa.; Martek Biosciences, Columbia, Md., etc.).

The assays to identify modulators are amenable to high throughput screening. Preferred assays thus detect enhancement or inhibition of bladder cancer gene transcription, inhibition or enhancement of polypeptide expression, and inhibition or enhancement of polypeptide activity.

High throughput assays for the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (e.g., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass.; etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors.

In a preferred embodiment, modulators are peptides of from about 5-30 amino acids, with from about 5-20 amino acids being preferred, and from about 7-15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate nucleotide or amino acid substitutions. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants. In a preferred embodiment, the library is biased. That is, some positions within the sequence are either held constant, or are selected from a limited number of possibilities. For example, in a preferred embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

Modulators of bladder cancer can also be nucleic acids, as defined above.

As described above generally for proteins, nucleic acid modulating agents may be naturally occurring nucleic acids, random nucleic acids, or “biased” random nucleic acids. Digests of procaryotic or eucaryotic genomes may be used as is outlined above for proteins.

In a preferred embodiment, the candidate compounds are organic chemical moieties, a wide variety of which are available in the literature.

After the candidate agent has been added and the cells allowed to incubate for some period of time, the sample containing a target sequence to be analyzed is added to the biochip. If required, the target sequence is prepared using known techniques. For example, the sample may be treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cy5.

In a preferred embodiment, the target sequence is labeled with, e.g., a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways. Components of the reaction may be added simultaneously, or sequentially, in different orders, with preferred embodiments outlined below. In addition, the reaction may include a variety of other reagents. These include salts, buffers, neutral proteins, e.g., albumin, detergents, etc., which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the bladder cancer phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens can be performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product.

In addition screens can be done for genes that are induced in response to a candidate agent. After identifying a modulator based upon its ability to suppress a bladder cancer expression pattern leading to a normal expression pattern, or to modulate a single bladder cancer gene expression profile so as to mimic the expression of the gene from normal tissue, a screen as described above can be performed to identify genes that are specifically modulated in response to the agent. Comparing expression profiles between normal tissue and agent treated bladder cancer tissue reveals genes that are not expressed in normal tissue or bladder cancer tissue, but are expressed in agent treated tissue. These agent-specific sequences can be identified and used by methods described herein for bladder cancer genes or proteins. In particular these sequences and the proteins they encode find use in marking or identifying agent treated cells. In addition, antibodies can be raised against the agent induced proteins and used to target novel therapeutics to the treated bladder cancer tissue sample.

Thus, in one embodiment, a test compound is administered to a population of bladder cancer cells, that have an associated bladder cancer expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (e.g., a peptide) may be put into a viral construct such as an adenoviral or retroviral construct, and added to the cell, such that expression of the peptide agent is accomplished, e.g., PCT US97/01019. Regulatable gene therapy systems can also be used.

Once the test compound has been administered to the cells, the cells can be washed if desired and are allowed to incubate under preferably physiological conditions for some period of time. The cells are then harvested and a new gene expression profile is generated, as outlined herein.

Thus, e.g., bladder cancer or non-malignant tissue may be screened for agents that modulate, e.g., induce or suppress the bladder cancer phenotype. A change in at least one gene, preferably many, of the expression profile indicates that the agent has an effect on bladder cancer activity. By defining such a signature for the bladder cancer phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “bladder cancer proteins” or a “bladder cancer modulatory protein”. The bladder cancer modulatory protein may be a fragment, or alternatively, be the full length protein to the fragment encoded by the nucleic acids of the Tables 1A-13. Preferably, the bladder cancer modulatory protein is a fragment. In a preferred embodiment, the bladder cancer amino acid sequence which is used to determine sequence identity or similarity is encoded by a nucleic acid of Tables 1A-13. In another embodiment, the sequences are naturally occurring allelic variants of a protein encoded by a nucleic acid of Tables 1A-13. In another embodiment, the sequences are sequence variants as further described herein.

Preferably, the bladder cancer modulatory protein is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the C-terminus of the fragment is kept as a free acid and the N-terminus is a free amine to aid in coupling, e.g., to cysteine.

In one embodiment the bladder cancer proteins are conjugated to an immunogenic agent as discussed herein. In one embodiment the bladder cancer protein is conjugated to BSA.

Measurements of bladder cancer polypeptide activity, or of bladder cancer or the bladder cancer phenotype can be performed using a variety of assays. For example, the effects of the test compounds upon the function of the bladder cancer polypeptides can be measured by examining parameters described above. A suitable physiological change that affects activity can be used to assess the influence of a test compound on the polypeptides of this invention. When the functional consequences are determined using intact cells or animals, one can also measure a variety of effects such as, in the case of bladder cancer associated with tumors, tumor growth, tumor metastasis, neovascularization, hormone release, transcriptional changes to both known and uncharacterized genetic markers (e.g., northern blots), changes in cell metabolism such as cell growth or pH changes, and changes in intracellular second messengers such as cGMP. In the assays of the invention, mammalian bladder cancer polypeptide is typically used, e.g., mouse, preferably human.

Assays to identify compounds with modulating activity can be performed in vitro. For example, a bladder cancer polypeptide is first contacted with a potential modulator and incubated for a suitable amount of time, e.g., from 0.5-48 hours. In one embodiment, the bladder cancer polypeptide levels are determined in vitro by measuring the level of protein or mRNA. The level of protein is measured using immunoassays such as western blotting, ELISA and the like with an antibody that selectively binds to the bladder cancer polypeptide or a fragment thereof. For measurement of mRNA, amplification, e.g., using PCR, LCR, or hybridization assays, e.g., northern hybridization, RNase protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using the bladder cancer protein promoter operably linked to a reporter gene such as luciferase, green fluorescent protein, CAT, or β-gal. The reporter construct is typically transfected into a cell. After treatment with a potential modulator, the amount of reporter gene transcription, translation, or activity is measured according to standard techniques known to those of skill in the art.

In a preferred embodiment, as outlined above, screens may be done on individual genes and gene products (proteins). That is, having identified a particular differentially expressed gene as important in a particular state, screening of modulators of the expression of the gene or the gene product itself can be done. The gene products of differentially expressed genes are sometimes referred to herein as “bladder cancer proteins.” The bladder cancer protein may be a fragment, or alternatively, be the full length protein to a fragment shown herein.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants can be further screened to better evaluate structure activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the bladder cancer proteins can be used in the assays.

Thus, in a preferred embodiment, the methods comprise combining a bladder cancer protein and a candidate compound, and determining the binding of the compound to the bladder cancer protein. Preferred embodiments utilize the human bladder cancer protein, although other mammalian proteins may also be used, e.g., for the development of animal models of human disease. In some embodiments, as outlined herein, variant or derivative bladder cancer proteins may be used.

Generally, in a preferred embodiment of the methods herein, the bladder cancer protein or the candidate agent is non-diffusably bound to an insoluble support having isolated sample receiving areas (e.g., a microtiter plate, an array, etc.). The insoluble supports may be made of a composition to which the compositions can be bound, is readily separated from soluble material, and is otherwise compatible with the overall method of screening. The surface of such supports may be solid or porous and of a convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the bladder cancer protein is bound to the support, and a test compound is added to the assay. Alternatively, the candidate agent is bound to the support and the bladder cancer protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the test modulating compound to the bladder cancer protein may be done in a number of ways. In a preferred embodiment, the compound is labeled, and binding determined directly, e.g., by attaching all or a portion of the bladder cancer protein to a solid support, adding a labeled candidate agent (e.g., a fluorescent label), washing off excess reagent, and determining whether the label is present on the solid support. Various blocking and washing steps may be utilized as appropriate.

In some embodiments, only one of the components is labeled, e.g., the proteins (or proteinaceous candidate compounds) can be labeled. Alternatively, more than one component can be labeled with different labels, e.g., ¹²⁵I for the proteins and a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (e.g., a bladder cancer protein), such as an antibody, peptide, binding partner, ligand, etc. Under certain circumstances, there may be competitive binding between the compound and the binding moiety, with the binding moiety displacing the compound. In one embodiment, the test compound is labeled. Either the compound, or the competitor, or both, is added first to the protein for a time sufficient to allow binding, if present. Incubations may be performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the bladder cancer protein and thus is capable of binding to, and potentially modulating, the activity of the bladder cancer protein. In this embodiment, either component can be labeled. Thus, e.g., if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor. The absence of binding by the competitor may indicate that the test compound is bound to the bladder cancer protein with a higher affinity. Thus, if the test compound is labeled, the presence of the label on the support, coupled with a lack of competitor binding, may indicate that the test compound is capable of binding to the bladder cancer protein.

In a preferred embodiment, the methods comprise differential screening to identity agents that are capable of modulating the activity of the bladder cancer proteins. In this embodiment, the methods comprise combining a bladder cancer protein and a competitor in a first sample. A second sample comprises a test compound, a bladder cancer protein, and a competitor. The binding of the competitor is determined for both samples, and a change, or difference in binding between the two samples indicates the presence of an agent capable of binding to the bladder cancer protein and potentially modulating its activity. That is, if the binding of the competitor is different in the second sample relative to the first sample, the agent is capable of binding to the bladder cancer protein.

Alternatively, differential screening is used to identify drug candidates that bind to the native bladder cancer protein, but cannot bind to modified bladder cancer proteins. The structure of the bladder cancer protein may be modeled, and used in rational drug design to synthesize agents that interact with that site. Drug candidates that affect the activity of a bladder cancer protein are also identified by screening drugs for the ability to either enhance or reduce the activity of the protein.

Positive controls and negative controls may be used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in an order that provides for the requisite binding.

In a preferred embodiment, the invention provides methods for screening for a compound capable of modulating the activity of a bladder cancer protein. The methods comprise adding a test compound, as defined above, to a cell comprising bladder cancer proteins. Many different cell types may be transfected to contain a recombinant nucleic acid that encodes a bladder cancer protein. In a preferred embodiment, a library of candidate agents are tested on a plurality of cells.

In one aspect, the assays are evaluated in the presence or absence or previous or subsequent exposure of physiological signals, e.g., hormones, antibodies, peptides, antigens, cytokines, growth factors, action potentials, and pharmacological agents including, e.g., chemotherapeutics, radiation, carcinogenics, or other cells (e.g., cell-cell contacts). In one example, the determinations are determined at different stages of the cell cycle process.

In this way, compounds that modulate bladder cancer agents are identified. Compounds with pharmacological activity are able to enhance or interfere with the activity of the bladder cancer protein. Once identified, similar structures are evaluated to identify critical structural feature of the compound.

In one embodiment, a method of inhibiting bladder cancer cell division is provided. The method comprises administration of a bladder cancer inhibitor. In another embodiment, a method of inhibiting bladder cancer is provided. The method comprises administration of a bladder cancer inhibitor. In a further embodiment, methods of treating cells or individuals with bladder cancer are provided. The method comprises administration of a bladder cancer inhibitor. In one embodiment, a bladder cancer inhibitor is an antibody as discussed above. In another embodiment, the bladder cancer inhibitor is an antisense molecule.

A variety of cell growth, proliferation, and metastasis assays are known to those of skill in the art, as described below.

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow. Soft agar growth or colony formation in suspension assays can be used to identify modulators of bladder cancer sequences, which when expressed in host cells, inhibit abnormal cellular proliferation and transformation. A therapeutic compound would reduce or eliminate the host cells' ability to grow in stirred suspension culture or suspended in semi-solid media, such as semi-solid or soft.

Techniques for soft agar growth or colony formation in suspension assays are described in Freshney (1994) Culture of Animal Cells: A Manual of Basic Technique (3d ed.) Wiley-Liss, herein incorporated by reference. See also, the methods section of Garkavtsev, et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with (3H)-thymidine at saturation density can be used to measure density limitation of growth. See Freshney (1994), supra. The transformed cells, when transfected with tumor suppressor genes, regenerate a normal phenotype and become contact inhibited and would grow to a lower density.

In this assay, labeling index with (³H)-thymidine at saturation density is a preferred method of measuring density limitation of growth. Transformed host cells are transfected with a bladder cancer-associated sequence and are grown for 24 hours at saturation density in non-limiting medium conditions. The percentage of cells labeling with (³H)-thymidine is determined autoradiographically. See, Freshney (1994), supra.

Growth Factor or Serum Dependence

Transformed cells have a lower serum dependence than their normal counterparts (see, e.g., Temin (1966) J. Nat'l Cancer Inst. 37:167-175; Eagle, et al. (1970) J. Exp. Med. 131:836-879; Freshney (1994), supra). This is in part due to release of various growth factors by the transformed cells. Growth factor or serum dependence of transformed host cells can be compared with that of control.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells. See, e.g., “Angiogenesis, tumor vascularization, and potential interference with tumor growth” pp. 178-184 in Mihich (ed. 1985) Biological Responses in Cancer Plenum. Similarly, tumor angiogenesis factor (TAF) is released at a higher level in tumor cells than their normal counterparts. See, e.g., Folkman (1992) Sem Cancer Biol. 3:89-96.

Various techniques which measure the release of these factors are described in Freshney (1994), supra. See also, Unkeless, et al. (1974) J. Biol. Chem. 249:4295-4305; Strickland and Beers (1976) J. Biol. Chem. 251:5694-5702; Whur, et al. (1980) Br. J. Cancer 42:305-312; Gullino “Angiogenesis, tumor vascularization, and potential interference with tumor growth” pp. 178-184 in Mihich (ed. 1985) Biological Responses in Cancer, Plenum; and Freshney (1985) Anticancer Res. 5:111-130.

Invasiveness into Matrigel

The degree of invasiveness into Matrigel or some other extracellular matrix constituent can be used as an assay to identify compounds that modulate bladder cancer-associated sequences. Tumor cells exhibit a good correlation between malignancy and invasiveness of cells into Matrigel or some other extracellular matrix constituent. In this assay, tumorigenic cells are typically used as host cells. Expression of a tumor suppressor gene in these host cells would decrease invasiveness of the host cells.

Techniques described in Freshney (1984), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (2000), supra.

Tumor Growth In Vivo

Effects of bladder cancer-associated sequences on cell growth can be tested in transgenic or immune-suppressed mice. Knock-out transgenic mice can be made, in which the bladder cancer gene is disrupted or in which a bladder cancer gene is inserted. Knock-out transgenic mice can be made by insertion of a marker gene or other heterologous gene into the endogenous bladder cancer gene site in the mouse genome via homologous recombination. Such mice can also be made by substituting the endogenous bladder cancer gene with a mutated version of the bladder cancer gene, or by mutating the endogenous bladder cancer gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. By breeding the chimeric mice it is possible to obtain a new line of mice containing the introduced genetic lesion. See, e.g., Capecchi, et al. (1989) Science 244:1288-1292. Chimeric targeted mice can be made. See Hogan, et al. (1988) Manipulating the Mouse Embryo: A Laboratory Manual, CSH Press; and Robertson (ed. 1987) Teratocarcinomas and Embryonic Stem Cells: A Practical Approach IRL Press, Washington, D.C.

Alternatively, various immune-suppressed or immune-deficient host animals can be used. For example, genetically athymic “nude” mouse (see, e.g., Giovanella, et al. (1974) J. Nat'l Cancer Inst. 52:921-930), a SCID mouse, a thymectomized mouse, or an irradiated mouse (see, e.g., Bradley, et al. (1978) Br. J. Cancer 38:263-272; Selby, et al. (1980) Br. J. Cancer 41:52-61) can be used as a host. Transplantable tumor cells (typically about 10⁶ cells) injected into isogenic hosts will produce invasive tumors in a high proportions of cases, while normal cells of similar origin will not. In hosts which developed invasive tumors, cells expressing a bladder cancer-associated sequences are injected subcutaneously. After a suitable length of time, preferably about 4-8 weeks, tumor growth is measured (e.g., by volume or by its two largest dimensions) and compared to the control. Tumors that have statistically significant reduction (using, e.g., Student's T test) are said to have inhibited growth.

Polynucleotide Modulators of Bladder Cancer

Antisense and RNAi Polynucleotides

In certain embodiments, the activity of a bladder cancer-associated protein is down-regulated, or entirely inhibited, by the use of antisense polynucleotide, e.g., a nucleic acid complementary to, and which can preferably hybridize specifically to, a coding mRNA nucleic acid sequence, e.g., a bladder cancer protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or intersugar linkages. Exemplary among these are the phosphorothioate and other sulfur containing species which are known for use in the art. Analogs are comprehended by this invention so long as they function effectively to hybridize with the bladder cancer protein mRNA. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known.

Antisense molecules as used herein include antisense or sense oligonucleotides. Sense oligonucleotides can, e.g., be employed to block transcription by binding to the antisense strand. The antisense and sense oligonucleotide comprise a single-stranded nucleic acid sequence (either RNA or DNA) capable of binding to target mRNA (sense) or DNA (antisense) sequences for bladder cancer molecules. A preferred antisense molecule is for a bladder cancer sequences in Tables 1A-13, or for a ligand or activator thereof. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, e.g., Stein and Cohen (1988) Cancer Res. 48:2659-2668; and van der Krol, et al. (1988) BioTechniques 6:958-976.

RNA interference is a mechanism to suppress gene expression in a sequence specific manner. See, e.g., Brumelkamp, et al. (2002) Sciencexpress (Mar. 21, 2002); Sharp (1999) Genes Dev. 13:139-141; and Cathew (2001) Curr. Op. Cell Biol. 13:244-248. In mammalian cells, short, e.g., 21 nt, double stranded small interfering RNAs (siRNA) have been shown to be effective at inducing an RNAi response. See, e.g., Elbashir, et al. (2001) Nature 411:494-498. The mechanism may be used to downregulate expression levels of identified genes, e.g., treatment of or validation of relevance to disease

Ribozymes

In addition to antisense polynucleotides, ribozymes can be used to target and inhibit transcription of bladder cancer-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes. See, e.g., Castanotto, et al. (1994) Adv. in Pharmacology 25: 289-317 for a general review of the properties of different ribozymes.

The general features of hairpin ribozymes are described, e.g., in Hampel, et al. (1990) Nucl. Acids Res. 18:299-304; European Patent Publication No. 0 360 257; U.S. Pat. No. 5,254,678. Methods of preparing them are well known. See, e.g., WO 94/26877; Ojwang, et al. (1993) Proc. Nat'l Acad. Sci. USA 90:6340-6344; Yamada, et al. (1994) Human Gene Therapy 1:39-45; Leavitt, et al. (1995) Proc. Nat'l Acad. Sci. USA 92:699-703; Leavitt, et al. (1994) Human Gene Therapy 5:1151-120; and Yamada, et al. (1994) Virology 205: 121-126.

Polynucleotide modulators of bladder cancer may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of bladder cancer may be introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment.

Thus, in one embodiment, methods of modulating bladder cancer in cells or organisms are provided. In one embodiment, the methods comprise administering to a cell an anti-bladder cancer antibody that reduces or eliminates the biological activity of an endogenous bladder cancer protein. Alternatively, the methods comprise administering to a cell or organism a recombinant nucleic acid encoding a bladder cancer protein. This may be accomplished in many ways. In a preferred embodiment, e.g., when the bladder cancer sequence is down-regulated in bladder cancer, such state may be reversed by increasing the amount of bladder cancer gene product in the cell. This can be accomplished, e.g., by overexpressing the endogenous bladder cancer gene or administering a gene encoding the bladder cancer sequence, using known gene-therapy techniques. In a preferred embodiment, the gene therapy techniques include the incorporation of the exogenous gene using enhanced homologous recombination (EHR), e.g., as described in PCT/US93/03868, hereby incorporated by reference in its entirety. Alternatively, e.g., when the bladder cancer sequence is up-regulated in bladder cancer, the activity of the endogenous bladder cancer gene is decreased, e.g., by the administration of a bladder cancer antisense nucleic acid.

In one embodiment, the bladder cancer proteins of the present invention may be used to generate polyclonal and monoclonal antibodies to bladder cancer proteins. Similarly, the bladder cancer proteins can be coupled, using standard technology, to affinity chromatography columns. These columns may then be used to purify bladder cancer antibodies useful for production, diagnostic, or therapeutic purposes. In a preferred embodiment, the antibodies are generated to epitopes unique to a bladder cancer protein; that is, the antibodies show little or no cross-reactivity to other proteins. The bladder cancer antibodies may be coupled to standard affinity chromatography columns and used to purify bladder cancer proteins. The antibodies may also be used as blocking polypeptides, as outlined above, since they will specifically bind to the bladder cancer protein.

Methods of Identifying Variant Bladder Cancer-Associated Sequences

Without being bound by theory, expression of various bladder cancer sequences is correlated with bladder cancer. Accordingly, disorders based on mutant or variant bladder cancer genes may be determined. In one embodiment, the invention provides methods for identifying cells containing variant bladder cancer genes, e.g., determining all or part of the sequence of at least one endogenous bladder cancer genes in a cell. This may be accomplished using many sequencing techniques. In a preferred embodiment, the invention provides methods of identifying the bladder cancer genotype of an individual, e.g., determining all or part of the sequence of at least one bladder cancer gene of the individual. This is generally done in at least one tissue of the individual, and may include the evaluation of a number of tissues or different samples of the same tissue. The method may include comparing the sequence of the sequenced bladder cancer gene to a known bladder cancer gene, e.g., a wild-type gene.

The sequence of all or part of the bladder cancer gene can then be compared to the sequence of a known bladder cancer gene to determine if differences exist. This can be done using many known homology programs, such as Bestfit, etc. In a preferred embodiment, the presence of a difference in the sequence between the bladder cancer gene of the patient and the known bladder cancer gene correlates with a disease state or a propensity for a disease state, as outlined herein.

In a preferred embodiment, the bladder cancer genes are used as probes to determine the number of copies of the bladder cancer gene in the genome.

In another preferred embodiment, the bladder cancer genes are used as probes to determine the chromosomal localization of the bladder cancer genes. Information such as chromosomal localization finds use in providing a diagnosis or prognosis in particular when chromosomal abnormalities such as translocations, and the like are identified in the bladder cancer gene locus.

Administration of Pharmaceutical and Vaccine Compositions

In one embodiment, a therapeutically effective dose of a bladder cancer protein or modulator thereof, is administered to a patient. By “therapeutically effective dose” herein is meant a dose that produces effects for which it is administered. The exact dose will depend on the purpose of the treatment, and will be ascertainable by one skilled in the art using known techniques. See, e.g., Ansel, et al. (1999) Pharmaceutical Dosage Forms and Drug Delivery Lippincott; Lieberman (1992) Pharmaceutical Dosage Forms (vols. 1-3) Dekker, ISBN 0824770846, 082476918X, 0824712692, 0824716981; Lloyd (1999) The Art, Science and Technology of Pharmaceutical Compounding Amer. Pharma. Assn.; and Pickar (1999) Dosage Calculations Thomson. As is known in the art, adjustments for bladder cancer degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. U.S. Ser. No. 09/687,576, further discloses the use of compositions and methods of diagnosis and treatment in bladder cancer and is hereby expressly incorporated by reference.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The administration of the bladder cancer proteins and modulators thereof of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, e.g., in the treatment of wounds and inflammation, the bladder cancer proteins and modulators may be directly applied as a solution or spray.

The pharmaceutical compositions of the present invention comprise a bladder cancer protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that bladder cancer protein modulators (e.g., antibodies, antisense constructs, ribozymes, small organic molecules, etc.) when administered orally, should be protected from digestion. This is typically accomplished either by complexing the molecule(s) with a composition to render it resistant to acidic and enzymatic hydrolysis, or by packaging the molecule(s) in an appropriately resistant carrier, such as a liposome or a protection barrier. Means of protecting agents from digestion are well known in the art.

The compositions for administration will commonly comprise a bladder cancer protein modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Hardman and Limbird (eds. 2001) Goodman and Gilman: The Pharmacologial Basis of Therapeutics McGraw-Hill.

Thus, a typical pharmaceutical composition for intravenous administration would be about 0.1-10 mg per patient per day. Dosages from about 0.1-100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington's Pharmaceutical Science and Goodman and Gilman: The Pharmacologial Basis of Therapeutics, supra.

The compositions containing modulators of bladder cancer proteins can be administered for therapeutic or prophylactic treatments. In therapeutic applications, compositions are administered to a patient suffering from a disease (e.g., a cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. The composition should provide a sufficient quantity of the agents of this invention to effectively treat the patient. An amount of modulator that is capable of preventing or slowing the development of cancer in a mammal is referred to as a “prophylactically effective dose.” The particular dose required for a prophylactic treatment will depend upon the medical condition and history of the mammal, the particular cancer being prevented, as well as other factors such as age, weight, gender, administration route, efficiency, etc. Such prophylactic treatments may be used, e.g., in a mammal who has previously had cancer to prevent a recurrence of the cancer, or in a mammal who is suspected of having a significant likelihood of developing cancer based, at least in part, upon gene expression profiles. Vaccine strategies may be used, in either a DNA vaccine form, or protein vaccine.

It will be appreciated that the present bladder cancer protein-modulating compounds can be administered alone or in combination with additional bladder cancer modulating compounds or with other therapeutic agent, e.g., other anti-cancer agents or treatments.

In numerous embodiments, one or more nucleic acids, e.g., polynucleotides comprising nucleic acid sequences set forth in Tables 1A-13, such as antisense polynucleotides or ribozymes, will be introduced into cells, in vitro or in vivo. The present invention provides methods, reagents, vectors, and cells useful for expression of bladder cancer-associated polypeptides and nucleic acids using in vitro (cell-free), ex vivo, or in vivo (cell or organism-based) recombinant expression systems.

The particular procedure used to introduce the nucleic acids into a host cell for expression of a protein or nucleic acid is application specific. Many procedures for introducing foreign nucleotide sequences into host cells may be used. These include the use of calcium phosphate transfection, spheroplasts, electroporation, liposomes, microinjection, plasma vectors, viral vectors, and other methods for introducing cloned genomic DNA, cDNA, synthetic DNA or other foreign genetic material into a host cell. See, e.g., Berger and Kimmel (1987) Guide to Molecular Cloning Techniques from Methods in Enzymology (vol.

152) Academic Press; Ausubel, et al. (eds. 1999 and supplements) Current Protocols in Molecular Biology Lippincott; and Sambrook, et al. (1989) Molecular Cloning: A Laboratory Manual (2d ed., Vol. 1-3) CSH Press.

In a preferred embodiment, bladder cancer proteins and modulators are administered as therapeutic agents, and can be formulated as outlined above. Similarly, bladder cancer genes (including both the full-length sequence, partial sequences, or regulatory sequences of the bladder cancer coding regions) can be administered in a gene therapy application. These bladder cancer genes can include antisense applications, either as gene therapy (e.g., for incorporation into the genome) or as antisense compositions, as will be appreciated by those in the art.

Bladder cancer polypeptides and polynucleotides can also be administered as vaccine compositions to stimulate HTL, CTL, and antibody responses. Such vaccine compositions can include, e.g., lipidated peptides (Vitiello, et al. (1995) J. Clin. Invest. 95:341-349); peptide compositions encapsulated in poly(DL-lactide-co-glycolide) (“PLG”) microspheres (Eldridge, et al. (1991) Molec. Immunol. 28:287-294; Alonso, et al. (1994) Vaccine 12:299-306; Jones, et al. (1995) Vaccine 13:675-681); peptide compositions contained in immune stimulating complexes (ISCOMS) (Takahashi, et al. (1990) Nature 344:873-875; Hu, et al. (1998) Clin. Exp. Immunol. 113:235-243); multiple antigen peptide systems (MAPs) (Tam (1988) Proc. Nat'l Acad. Sci. USA 85:5409-5413; Tam (1996) J. Immunol. Methods 196:17-32); peptides formulated as multivalent peptides; peptides for use in ballistic delivery systems, typically crystallized peptides, viral delivery vectors (Perkus, et al. in Kaufmann (ed. 1996) Concepts in Vaccine Development de Gruyter; Chakrabarti, et al. (1986) Nature 320:535-537; Hu, et al. (1986) Nature 320:537-547; Kieny, et al. (1986) AIDS Bio/Technology4:790; Top, et al. (1971) J. Infect. Dis. 124:148-154; Chanda, et al. (1990) Virology 175:535-547), particles of viral or synthetic origin (see, e.g., Kofler, et al. (1996) J. Immunol. Methods 192:25-35; Eldridge, et al. (1993) Sem. Hematol. 30:16-24; Falo, et al. (1995) Nature Med. 7:649-653), adjuvants (Warren, et al. (1986) Annu. Rev. Immunol. 4:369-388; Gupta, et al. (1993) Vaccine 11:293-306), liposomes (Reddy, et al. (1992) J. Immunol. 148:1585-1589; Rock (1996) Immunol. Today 17:131-137), or, naked or particle absorbed cDNA (Ulmer, et al. (1993) Science 259:1745-1749; Robinson, et al. (1993) Vaccine 11:957-960; Shiver, et al. in Kaufmann (ed. 1996) Concepts in Vaccine Development de Gruyter; Cease and Berzofsky (1994) Annu. Rev. Immunol. 12:923-989; and Eldridge, et al. (1993) Sem. Hematol. 30:16-24). Toxin-targeted delivery technologies, also known as receptor mediated targeting, such as those of Avant Immunotherapeutics, Inc., Needham, Mass., may also be used.

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, e.g., Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2,-7,-12, and other like growth factors, may also be used as adjuvants.

Vaccines can be administered as nucleic acid compositions wherein DNA or RNA encoding one or more of the polypeptides, or a fragment thereof, is administered to a patient. This approach is described, for instance, in Wolff, et al. (1990) Science 247:1465-1468 as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, e.g., as a vector to express nucleotide sequences that encode bladder cancer polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover, et al. (1991) Nature 351:456-460. A wide variety of other vectors useful for therapeutic administration or immunization, e.g., adeno and adeno-associated virus vectors, retroviral vectors, Salmonella typhi vectors, detoxified anthrax toxin vectors, and the like. See, e.g., Shata, et al. (2000) Mol Med Today 6:66-71; Shedlock, et al. (2000) J. Leukoc. Biol. 68:793-806; Hipp, et al. (2000) In Vivo 14:571-85.

Methods for the use of genes as DNA vaccines are well known, and include placing a bladder cancer gene or portion of a bladder cancer gene under the control of a regulatable promoter or a tissue-specific promoter for expression in a bladder cancer patient. The bladder cancer gene used for DNA vaccines can encode full-length bladder cancer proteins, but more preferably encodes portions of the bladder cancer proteins including peptides derived from the bladder cancer protein. In one embodiment, a patient is immunized with a DNA vaccine comprising a plurality of nucleotide sequences derived from a bladder cancer gene. For example, bladder cancer-associated genes or sequence encoding subfragments of a bladder cancer protein are introduced into expression vectors and tested for their immunogenicity in the context of Class I MHC and an ability to generate cytotoxic T cell responses. This procedure provides for production of cytotoxic T cell responses against cells which present antigen, including intracellular epitopes.

In a preferred embodiment, the DNA vaccines include a gene encoding an adjuvant molecule with the DNA vaccine. Such adjuvant molecules include cytokines that increase the immunogenic response to the bladder cancer polypeptide encoded by the DNA vaccine. Additional or alternative adjuvants are available.

In another preferred embodiment bladder cancer genes find use in generating animal models of bladder cancer. When the bladder cancer gene identified is repressed or diminished in cancer tissue, gene therapy technology, e.g., wherein antisense RNA directed to the bladder cancer gene will also diminish or repress expression of the gene. Animal models of bladder cancer find use in screening for modulators of a bladder cancer-associated sequence or modulators of bladder cancer. Similarly, transgenic animal technology including gene knockout technology, e.g., as a result of homologous recombination with an appropriate gene targeting vector, will result in the absence or increased expression of the bladder cancer protein. When desired, tissue-specific expression or knockout of the bladder cancer protein may be necessary.

It is also possible that the bladder cancer protein is overexpressed in bladder cancer. As such, transgenic animals can be generated that overexpress the bladder cancer protein. Depending on the desired expression level, promoters of various strengths can be employed to express the transgene. Also, the number of copies of the integrated transgene can be determined and compared for a determination of the expression level of the transgene. Animals generated by such methods find use as animal models of bladder cancer and are additionally useful in screening for modulators to treat bladder cancer.

Kits for Use in Diagnostic and/or Prognostic Applications

For use in diagnostic, research, and therapeutic applications suggested above, kits are also provided by the invention. In the diagnostic and research applications such kits may include one or more of the following: assay reagents, buffers, bladder cancer-specific nucleic acids or antibodies, hybridization probes and/or primers, antisense or inhibitory polynucleotides, ribozymes, dominant negative bladder cancer polypeptides or polynucleotides, small molecules inhibitors of bladder cancer-associated sequences etc. A therapeutic product may include sterile saline or another pharmaceutically acceptable emulsion and suspension base.

In addition, the kits may include instructional materials containing directions (e.g., protocols) for the practice of the methods of this invention. While the instructional materials typically comprise written or printed materials they are not limited to such. A medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of bladder cancer-associated sequences. Such kits can be prepared from readily available materials and reagents. For example, such kits can comprise one or more of the following materials: a bladder cancer-associated polypeptide or polynucleotide, control positive or negative samples, reaction tubes, and instructions for testing bladder cancer-associated activity. Optionally, the kit contains biologically active bladder cancer protein. A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.

### EXAMPLES

### Example 1

### Gene Chip Analysis

Molecular profiles of various normal and cancerous tissues were determined and analyzed using gene chips. RNA was isolated and gene chip analysis was performed as described (Glynne, et al. (2000) Nature 403:672-676; Zhao, et al. (2000) Genes Dev. 14:981-993).

### Table Descriptions

Table 1A shows about 3413 that exhibit increased or decreased expression in bladder cancer samples. See U.S. S No. 60/302,814.

Table 2A shows about 485 genes overexpressed in bladder tumors relative to normal tissues as analyzed using the Affymetrix/Eos Hu03 GeneChip array. See U.S. S No. 60/343,705.

Table 3A shows about 414 genes upregulated in bladder cancer relative to normal body tissues and preferred for utility as small molecule, antibody, DNA vaccine targets for the therapy of bladder cancer. These genes were selected from 59680 probesets on the Eos/Affymetrix Hu03 Genechip array. Gene expression data for each probeset obtained from this analysis was expressed as average intensity (AI), a normalized value reflecting the relative level of mRNA expression.

Table 4A shows about 129 genes upregulated in bladder cancer relative to normal body tissues and preferred for utility as diagnostics of bladder cancer. These genes were selected from 59680 probesets on the Eos/Affymetrix Hu03 Genechip array. Gene expression data for each probeset obtained from this analysis was expressed as average intensity (AI), a normalized value reflecting the relative level of mRNA expression.

Table 5A shows about 149 genes upregulated in bladder cancer relative to normal body tissues. These genes were selected from 59680 probesets on the Eos/Affymetrix Hu03 Genechip array. Gene expression data for each probeset obtained from this analysis was expressed as average intensity (AI), a normalized value reflecting the relative level of mRNA expression.

Table 6A shows about 199 genes upregulated in bladder cancer relative to normal bladder tissue. These genes were selected from 59680 probesets on the Eos/Affymetrix Hu03 Genechip array. Gene expression data for each probeset obtained from this analysis was expressed as average intensity (AI), a normalized value reflecting the relative level of mRNA expression.

Table 7A shows about 63 genes downregulated in bladder tumors relative to normal bladder. These genes were selected from 59680 probesets on the Eos/Affymetrix Hu03 Genechip array. Gene expression data for each probeset obtained from this analysis was expressed as average intensity (AI), a normalized value reflecting the relative level of mRNA expression.

Table 8A shows about 1440 genes upregulated in Ta or Ti bladder tumors from patients who later presented with muscle-invasive bladder tumors (stage T2-T4). Gene expression data for each probeset obtained from this analysis was expressed as average intensity (AI), a normalized value reflecting the relative level of mRNA expression.

Table 9A shows about 1200 genes upregulated in Ta or TI tumors of patients who later presented with either more Ta tumors or no tumors at all. Gene expression data for each probeset obtained from this analysis was expressed as average intensity (AI), a normalized value reflecting the relative level of mRNA expression.

Table 10A shows about 65 genes upregulated in non-invasive exophytic Ta bladder tumors relative to T2-T4 muscle-invasive tumors. Gene expression data for each probeset obtained from this analysis was expressed as average intensity (AI), a normalized value reflecting the relative level of mRNA expression.

Table 11A shows about 106 genes upregulated in muscle-invasive T2-T4 bladder tumors relative to non-invasive exophytic Ta bladder tumors. Gene expression data for each probeset obtained from this analysis was expressed as average intensity (AI), a normalized value reflecting the relative level of mRNA expression.

Table 12A shows the Pkey, ExAccn, UnigeneID, and Unigene Title for all of the sequences in Table 13. Seq ID No. is used to link Table 12A to table 13.

Tables 1B-12B show the accession numbers for those Pkey's lacking UnigeneID's for tables 1A-12A, respectively. For each probeset is listed a gene cluster number from which oligonucleotides were designed. Gene clusters were compiled using sequences derived from Genbank ESTs and mRNAs. These sequences were clustered based on sequence similarity using Clustering and Alignment Tools (DoubleTwist, Oakland Calif.). Genbank accession numbers for sequences comprising each cluster are listed in the “Accession” column.

Tables 1C-12C show genomic positioning for Pkey's lacking Unigene ID's and accession numbers for tables 1A-12A, respectively. For each predicted exon, is listed genomic sequence source used for prediction. Nucleotide locations of each predicted exon are also listed.

TABLE 1A

| Pkey | ExAccn | UnigeneID | Unigene Title | R1 | R2 | Target Type |
|---|---|---|---|---|---|---|
| 400440 | X83957 | Hs.83870 | nebulin | 0.17 | 2.05 | downregulate stage |
| 400888 | | | | 0.24 | 1.97 | downregulate stage |
| 401566 | | | | 0.19 | 4.06 | downregulate stage |
| 401669 | | | | 0.2 | 2.05 | downregulate stage |
| 401691 | | | | 0.04 | 10.13 | downregulate stage |
| 401905 | | | | 0.3 | 1.87 | downregulate stage |
| 402076 | | | | 0.06 | 6.51 | downregulate stage |
| 402110 | | | | 0.43 | 2.35 | downregulate stage |
| 402271 | NM_002197 | Hs.154721 | aconitase 1, soluble | 0.21 | 2.16 | downregulate stage |
| 403362 | | | | 0.18 | 4.44 | downregulate stage |
| 403687 | | | | 0.32 | 1.91 | downregulate stage |
| 403959 | | | | 0.14 | 2.27 | downregulate stage |
| 404015 | | | | 0.2 | 2.48 | downregulate stage |
| 404059 | | | | 0.36 | 1.84 | downregulate stage |
| 404152 | | | | 0.32 | 1.85 | downregulate stage |
| 404498 | | | | 0.14 | 2.18 | downregulate stage |
| 404819 | | | | 0.19 | 5.25 | downregulate stage |
| 405001 | U58196 | Hs.296281 | interleukin enhancer binding factor 1 | 0.16 | 2.92 | downregulate stage |
| 405349 | | | | 0.18 | 3.8 | downregulate stage |
| 405390 | | | | 0.3 | 2.54 | downregulate stage |
| 405735 | | | | 0.13 | 2.44 | downregulate stage |
| 405968 | | | | 0.26 | 1.85 | downregulate stage |
| 406017 | | | | 0.32 | 2.28 | downregulate stage |
| 406305 | BE261320 | Hs.297096 | transcriptional adaptor 3 (ADA3, yeast h | 0.42 | 1.93 | downregulate stage |
| 406320 | | | | 0.37 | 2.01 | downregulate stage |
| 406704 | M21665 | Hs.929 | myosin, heavy polypeptide 7, cardiac mus | 0.3 | 2.84 | downregulate stage |
| 406706 | X03740 | Hs.231581 | myosin, heavy polypeptide 1, skeletal mu | 0.14 | 7.4 | downregulate stage |
| 406707 | S73840 | Hs.931 | myosin, heavy polypeptide 2, skeletal mu | 0.05 | 12.51 | downregulate stage |
| 407013 | U35637 | | gb: Human nebulin mRNA, partial cds | 0.14 | 2.17 | downregulate stage |
| 407245 | X90568 | Hs.172004 | titin | 0.02 | 15.21 | downregulate stage |
| 407330 | AA582607 | | gb: nn51b05.s1 NCI_CGAP_Kid6 Homo sapiens | 0.42 | 1.87 | downregulate stage |
| 407571 | AI446183 | Hs.9572 | ESTs | 0.38 | 2.13 | downregulate stage |
| 407682 | AL035858 | Hs.3807 | FXYD domain-containing ion transport reg | 0.34 | 2.56 | downregulate stage |
| 407815 | AW373860 | Hs.301716 | ESTs | 0.31 | 2.44 | downregulate stage |
| 407834 | AW084991 | Hs.26100 | ESTs | 0.15 | 2.98 | downregulate stage |
| 407891 | AA486620 | Hs.41135 | endomucin-2 | 0.15 | 3.33 | downregulate stage |
| 407906 | AA369665 | Hs.41185 | Homo sapiens mRNA; cDNA DKFZp564O1262 (f | 0.12 | 8.05 | downregulate stage |
| 407938 | AA905097 | Hs.85050 | phospholamban | 0.08 | 8.77 | downregulate stage |
| 407965 | W21483 | Hs.41707 | heat shock 27 kD protein 3 | 0.26 | 2.29 | downregulate stage |
| 408009 | AF020498 | Hs.41735 | purinergic receptor P2X, ligand-gated io | 0.49 | 1.91 | downregulate stage |
| 408139 | AA451966 | Hs.43005 | RAB9-like protein | 0.41 | 1.88 | downregulate stage |
| 408221 | AA912183 | Hs.47447 | ESTs | 0.04 | 24.1 | downregulate stage |
| 408374 | AW025430 | Hs.155591 | forkhead box F1 | 0.35 | 2.85 | downregulate stage |
| 408493 | BE206854 | Hs.46039 | phosphoglycerate mutase 2 (muscle) | 0.09 | 9.04 | downregulate stage |
| 408508 | AI806109 | Hs.135736 | KIAA1580 protein | 0.45 | 2.2 | downregulate stage |
| 408614 | AL137698 | Hs.46531 | Homo sapiens mRNA; cDNA DKFZp434C1915 (f | 0.13 | 5.48 | downregulate stage |
| 408652 | R43409 | Hs.6829 | ESTs | 0.33 | 2.5 | downregulate stage |
| 408753 | AI337192 | Hs.47438 | SH3 domain binding glutamic acid-rich pr | 0.05 | 6.94 | downregulate stage |
| 408896 | AI610447 | Hs.48778 | niban protein | 0.39 | 2.01 | downregulate stage |
| 409277 | T05558 | Hs.156880 | ESTs | 0.18 | 2.75 | downregulate stage |
| 410023 | AB017169 | Hs.57929 | slit (Drosophila) homolog 3 | 0.07 | 2.7 | downregulate stage |
| 410036 | R57171 | Hs.57975 | calsequestrin 2, cardiac muscle | 0.09 | 5.23 | downregulate stage |
| 410132 | NM_003480 | Hs.58882 | Microfibril-associated glycoprotein-2 | 0.24 | 2.34 | downregulate stage |
| 410168 | AW834050 | Hs.9973 | tensin | 0.39 | 2.17 | downregulate stage |
| 410243 | D83402 | Hs.289006 | ESTs, Weakly similar to alternatively sp | 0.11 | 2.82 | downregulate stage |
| 410339 | AI916499 | Hs.298258 | ESTs | 0.15 | 2.16 | downregulate stage |
| 410677 | NM_003278 | Hs.65424 | tetranectin (plasminogen-binding protein | 0.32 | 2.65 | downregulate stage |
| 410868 | T06529 | Hs.98518 | Homo sapiens cDNA FLJ11490 fis, clone HE | 0.2 | 2.74 | downregulate stage |
| 411048 | AK001742 | Hs.67991 | hypothetical protein DKFZp434G0522 | 0.2 | 1.92 | downregulate stage |
| 411067 | AI681006 | Hs.301543 | ESTs | 0.11 | 3.41 | downregulate stage |
| 411069 | AL133092 | Hs.68055 | hypothetical protein DKFZp434I0428 | 0.17 | 5.8 | downregulate stage |
| 411644 | H92064 | Hs.301548 | ESTs | 0.06 | 13.8 | downregulate stage |
| 411741 | AW859650 | | gb: RC0-CT0358-071299-011-d03 CT0358 Homo | 0.36 | 2.5 | downregulate stage |
| 412047 | AA934589 | Hs.49696 | ESTs | 0.18 | 3.57 | downregulate stage |
| 412095 | AI624707 | Hs.5921 | Homo sapiens cDNA: FLJ21592 fis, clone C | 0.32 | 1.89 | downregulate stage |
| 412389 | AW947655 | | gb: RC0-MT0003-140300-031-b07 MT0003 Homo | 0.38 | 2.6 | downregulate stage |
| 412442 | AI983730 | Hs.26530 | serum deprivation response (phosphatidyl | 0.12 | 3.67 | downregulate stage |
| 412519 | AA196241 | Hs.73980 | troponin T1, skeletal, slow | 0.24 | 1.86 | downregulate stage |
| 412622 | AW664708 | Hs.171959 | ESTs | 0.06 | 5.45 | downregulate stage |
| 412649 | NM_002206 | Hs.74369 | integrin, alpha 7 | 0.29 | 2.95 | downregulate stage |
| 412659 | AW753865 | Hs.74376 | olfactomedin related ER localized protei | 0.18 | 2.06 | downregulate stage |
| 412758 | Y07818 | Hs.74566 | dihydropyrimidinase-like 3 | 0.3 | 2.23 | downregulate stage |
| 412802 | U41518 | Hs.74602 | aquaporin 1 (channel-forming integral pr | 0.11 | 2.71 | downregulate stage |
| 412975 | T70956 | Hs.75106 | clusterin (complement lysis inhibitor, S | 0.44 | 2.03 | downregulate stage |
| 413074 | AI871368 | Hs.8417 | ESTs | 0.47 | 1.91 | downregulate stage |
| 413272 | AA127923 | Hs.293256 | ESTs | 0.09 | 4.44 | downregulate stage |
| 413276 | Z24725 | Hs.75260 | mitogen inducible 2 | 0.23 | 2.48 | downregulate stage |
| 413508 | BE145364 | | gb: IL0-HT0198-151099-125-e05 HT0198 Homo | 0.31 | 2.53 | downregulate stage |
| 413624 | BE177019 | Hs.75445 | SPARC-like 1 (mast9, hevin) | 0.33 | 2.17 | downregulate stage |
| 413778 | AA090235 | Hs.75535 | myosin, light polypeptide 2, regulatory, | 0.33 | 2.63 | downregulate stage |
| 414063 | H26904 | Hs.75736 | apolipoprotein D | 0.42 | 1.85 | downregulate stage |
| 414241 | AA425085 | Hs.4007 | Sarcolemmal-associated protein | 0.16 | 2.22 | downregulate stage |
| 414290 | AI568801 | Hs.71721 | ESTs | 0.02 | 10 | downregulate stage |
| 414629 | AA345824 | Hs.76688 | carboxylesterase 1 (monocyte/macrophage | 0.13 | 4.14 | downregulate stage |
| 414657 | AA424074 | Hs.76780 | protein phosphatase 1, regulatory (inhib | 0.33 | 2.14 | downregulate stage |
| 414712 | N88858 | Hs.77039 | ribosomal protein S3A | 0.4 | 2.5 | downregulate stage |
| 414903 | AA451700 | Hs.85835 | Homo sapiens cDNA: FLJ22841 fis, clone K | 0.3 | 3.3 | downregulate stage |
| 415165 | AW887604 | Hs.78065 | complement component 7 | 0.04 | 3.41 | downregulate stage |
| 415274 | AF001548 | Hs.78344 | myosin, heavy polypeptide 11, smooth mus | 0.2 | 3.29 | downregulate stage |
| 415447 | Z97171 | Hs.78454 | myocilin, trabecular meshwork inducible | 0.15 | 6.55 | downregulate stage |
| 415672 | N53097 | Hs.193579 | ESTs | 0.28 | 3.55 | downregulate stage |
| 415934 | NM_000928 | Hs.992 | phospholipase A2, group IB (pancreas) | 0.34 | 2.64 | downregulate stage |
| 416127 | N49843 | Hs.79022 | GTP-binding protein overexpressed in ske | 0.3 | 1.98 | downregulate stage |
| 416349 | X69089 | Hs.79227 | myomesin (M-protein) 2 (165 kD) | 0.41 | 1.96 | downregulate stage |
| 416585 | X54162 | Hs.79386 | leiomodin 1 (smooth muscle) | 0.02 | 49.3 | downregulate stage |
| 416854 | H40164 | Hs.80296 | Purkinje cell protein 4 | 0.02 | 7.55 | downregulate stage |
| 416941 | BE000150 | Hs.48778 | niban protein | 0.27 | 2.16 | downregulate stage |
| 416982 | J05401 | Hs.80691 | creatine kinase, mitochondrial 2 (sarcom | 0.29 | 2.43 | downregulate stage |
| 417011 | F08212 | Hs.234898 | ESTs | 0.41 | 2.06 | downregulate stage |
| 417298 | AW665639 | Hs.37958 | ESTs | 0.27 | 3.7 | downregulate stage |
| 417501 | AL041219 | Hs.82222 | sema domain, immunoglobulin domain (lg), | 0.39 | 2.08 | downregulate stage |
| 417553 | L09190 | Hs.82276 | trichohyalin | 0.29 | 2.59 | downregulate stage |
| 417987 | AA210872 | Hs.50133 | ESTs | 0.22 | 2.09 | downregulate stage |
| 418297 | R91254 | | gb: yp94e12.s1 Soares fetal liver spleen | 0.28 | 1.9 | downregulate stage |
| 418332 | R34976 | Hs.78293 | ESTs | 0.2 | 3.9 | downregulate stage |
| 418391 | NM_003281 | Hs.84673 | troponin I, skeletal, slow | 0.35 | 2.02 | downregulate stage |
| 418409 | AA219332 | Hs.120869 | ESTs, Weakly similar to AF092922 1 retin | 0.21 | 3.8 | downregulate stage |
| 418421 | R58620 | Hs.85050 | phospholamban | 0.2 | 2.08 | downregulate stage |
| 418489 | U76421 | Hs.85302 | adenosine deaminase, RNA-specific, B1 (h | 0.05 | 21.55 | downregulate stage |
| 418533 | NM_004533 | Hs.85937 | myosin-binding protein C, fast-type | 0.42 | 1.95 | downregulate stage |
| 418787 | AW296134 | Hs.86999 | ESTs | 0.48 | 1.87 | downregulate stage |
| 418793 | AW382987 | Hs.88474 | prostaglandin-endoperoxide synthase 1 (p | 0.26 | 2.43 | downregulate stage |
| 418947 | W52990 | Hs.22860 | ESTs | 0.13 | 7.18 | downregulate stage |
| 419037 | R39895 | Hs.7864 | ESTs | 0.27 | 2 | downregulate stage |
| 419441 | AW023731 | Hs.274368 | Homo sapiens mRNA; cDNA DKFZp586I1524 (f | 0.19 | 5.25 | downregulate stage |
| 419535 | AW139550 | Hs.115173 | ESTs | 0.31 | 2.59 | downregulate stage |
| 419682 | H13139 | Hs.92282 | paired-like homeodomain transcription fa | 0.28 | 2.38 | downregulate stage |
| 419685 | W76083 | Hs.173077 | ESTs | 0.4 | 2.21 | downregulate stage |
| 419703 | AI793257 | Hs.128151 | ESTs | 0.09 | 3.52 | downregulate stage |
| 419942 | U25138 | Hs.93841 | potassium large conductance calcium-acti | 0.28 | 2.96 | downregulate stage |
| 420058 | AK001423 | Hs.94694 | Homo sapiens cDNA FLJ10561 fis, clone NT | 0.3 | 2.09 | downregulate stage |
| 420195 | N44348 | Hs.300794 | ESTs | 0.22 | 2.79 | downregulate stage |
| 420261 | AW206093 | Hs.748 | fibroblast growth factor receptor 1 (fms | 0.35 | 1.98 | downregulate stage |
| 420674 | NM_000055 | Hs.1327 | butyrylcholinesterase | 0.29 | 3.5 | downregulate stage |
| 421296 | NM_002666 | Hs.103253 | perilipin | 0.36 | 2.11 | downregulate stage |
| 421639 | NM_012082 | Hs.297921 | Homo sapiens mRNA full length insert cDN | 0.13 | 4.3 | downregulate stage |
| 421763 | AW163500 | Hs.108080 | cysteine and glycine-rich protein 1 | 0.26 | 3.49 | downregulate stage |
| 421853 | AL117472 | Hs.108924 | DKFZP586P1422 protein | 0.14 | 5 | downregulate stage |
| 422103 | AA984330 | Hs.111676 | protein kinase H11; small stress protein | 0.2 | 2.29 | downregulate stage |
| 422287 | F16365 | Hs.114346 | cytochrome c oxidase subunit VIIa polype | 0.27 | 2.58 | downregulate stage |
| 422320 | AI745249 | Hs.23650 | ESTs, Weakly similar to AAB47496 NG5 [H. | 0.24 | 2.95 | downregulate stage |
| 422633 | X56832 | Hs.118804 | enolase 3, (beta, muscle) | 0.23 | 3.57 | downregulate stage |
| 422639 | AI929377 | Hs.173724 | creatine kinase, brain | 0.39 | 1.97 | downregulate stage |
| 423334 | AK000906 | Hs.127273 | hypothetical protein FLJ10044 | 0.37 | 2.29 | downregulate stage |
| 423587 | AA328074 | Hs.284256 | hypothetical protein FLJ14033 similar to | 0.37 | 2.47 | downregulate stage |
| 423889 | AL035447 | Hs.134594 | hypothetical protein LOC57158 | 0.24 | 2.43 | downregulate stage |
| 424181 | AL039482 | Hs.142517 | Homo sapiens mRNA; cDNA DKFZp434P0810 (f | 0.27 | 2.28 | downregulate stage |
| 424206 | NM_003734 | Hs.198241 | amine oxidase, copper containing 3 (vasc | 0.3 | 2.59 | downregulate stage |
| 424479 | AF064238 | Hs.149098 | smoothelin | 0.26 | 3.29 | downregulate stage |
| 424580 | AA446539 | Hs.35092 | ESTs | 0.15 | 2.57 | downregulate stage |
| 424846 | AU077324 | Hs.1832 | neuropeptide Y | 0.4 | 2.04 | downregulate stage |
| 424938 | AW102607 | Hs.245233 | ESTs | 0.29 | 2.16 | downregulate stage |
| 424982 | U94777 | Hs.154084 | phosphorylase, glycogen; muscle (McArdle | 0.42 | 1.89 | downregulate stage |
| 425383 | D83407 | Hs.156007 | Down syndrome critical region gene 1-lik | 0.14 | 1.86 | downregulate stage |
| 425545 | N98529 | Hs.158295 | Human mRNA for myosin light chain 3 (MLC | 0.03 | 13.25 | downregulate stage |
| 425622 | AW360847 | Hs.16578 | ESTs | 0.3 | 2.19 | downregulate stage |
| 425751 | T19239 | Hs.1940 | crystallin, alpha B | 0.47 | 1.92 | downregulate stage |
| 425869 | AA524547 | Hs.160318 | FXYD domain-containing ion transport reg | 0.45 | 1.85 | downregulate stage |
| 425887 | AL049443 | Hs.161283 | Homo sapiens mRNA; cDNA DKFZp586N2020 (f | 0.19 | 2.85 | downregulate stage |
| 425932 | M81650 | Hs.1968 | semenogelin I | 0.02 | 16.3 | downregulate stage |
| 426354 | NM_004010 | Hs.169470 | dystrophin (muscular dystrophy, Duchenne | 0.27 | 2.52 | downregulate stage |
| 426429 | X73114 | Hs.169849 | myosin-binding protein C, slow-type | 0.1 | 10.3 | downregulate stage |
| 426752 | X69490 | Hs.172004 | titin | 0.03 | 31.3 | downregulate stage |
| 426809 | BE313114 | Hs.29706 | ESTs | 0.34 | 2.95 | downregulate stage |
| 427078 | AI676062 | Hs.111902 | ESTs | 0.22 | 2.11 | downregulate stage |
| 427136 | AL117415 | Hs.173716 | Homo sapiens mRNA; cDNA DKFZp434K0521 (f | 0.37 | 2.33 | downregulate stage |
| 427164 | AB037721 | Hs.173871 | KIAA1300 protein | 0.12 | 5.47 | downregulate stage |
| 427185 | AA398930 | Hs.186674 | ESTs | 0.22 | 4.65 | downregulate stage |
| 427373 | AB007972 | Hs.177533 | Homo sapiens mRNA; cDNA DKFZp586N0318 (f | 0.22 | 3.18 | downregulate stage |
| 427393 | AB029018 | Hs.177635 | KIAA1095 protein | 0.27 | 2.13 | downregulate stage |
| 427665 | AF134803 | Hs.180141 | cofilin 2 (muscle) | 0.05 | 4 | downregulate stage |
| 427676 | AA394062 | Hs.180266 | tropomyosin 2 (beta) | 0.45 | 1.87 | downregulate stage |
| 427888 | AA417088 | Hs.137598 | ESTs | 0.36 | 2.04 | downregulate stage |
| 427980 | AA418305 | | gb: zv96g05.s1 Soares_NhHMPu_S1 Home sapi | 0.32 | 2.39 | downregulate stage |
| 428087 | AA100573 | Hs.182421 | troponin C2, fast | 0.17 | 4.37 | downregulate stage |
| 428138 | AA773842 | Hs.293799 | ESTs | 0.45 | 2.2 | downregulate stage |
| 428221 | U96781 | Hs.183075 | ESTs, Highly similar to Ca2+ ATPase of f | 0.23 | 3.36 | downregulate stage |
| 428329 | AA426091 | Hs.98453 | ESTs | 0.21 | 2.09 | downregulate stage |
| 428409 | AW117207 | Hs.98523 | ESTs | 0.1 | 7.63 | downregulate stage |
| 428411 | AW291464 | Hs.10338 | ESTs | 0.32 | 1.98 | downregulate stage |
| 428648 | AF052728 | Hs.188021 | potassium voltage-gated channel, subfami | 0.08 | 2.99 | downregulate stage |
| 428649 | AL045716 | Hs.188228 | Homo sapiens cDNA FLJ11003 fis, clone PL | 0.11 | 2.07 | downregulate stage |
| 428899 | AA744610 | Hs.194431 | palladin | 0.42 | 1.84 | downregulate stage |
| 429350 | AI754634 | Hs.131987 | ESTs | 0.06 | 4.73 | downregulate stage |
| 429525 | N92540 | Hs.205353 | ectonucleoside triphosphate diphosphohyd | 0.18 | 2.31 | downregulate stage |
| 429545 | AI824164 | Hs.77667 | lymphocyte antigen 6 complex, locus E | 0.31 | 2.07 | downregulate stage |
| 429655 | U48959 | Hs.211582 | myosin, light polypeptide kinase | 0.33 | 2.18 | downregulate stage |
| 429892 | NM_003803 | Hs.2504 | myomesin 1 (skelemin) (185 kD) | 0.36 | 2.17 | downregulate stage |
| 429930 | AI580809 | Hs.99569 | ESTs | 0.18 | 5.6 | downregulate stage |
| 429956 | AI374651 | Hs.22542 | ESTs | 0.22 | 4.45 | downregulate stage |
| 430013 | AA463833 | Hs.151275 | ESTs | 0.21 | 3.03 | downregulate stage |
| 430271 | T06199 | Hs.237506 | heat shock cognate 40 | 0.47 | 1.85 | downregulate stage |
| 430310 | U60115 | Hs.239069 | four and a half LIM domains 1 | 0.18 | 3.44 | downregulate stage |
| 430418 | R98852 | Hs.36029 | heart and neural crest derivatives expre | 0.38 | 2.26 | downregulate stage |
| 430699 | AW969847 | Hs.292718 | ESTs, Weakly similar to RET2_HUMAN RETIN | 0.16 | 2.52 | downregulate stage |
| 430712 | AW044647 | Hs.196284 | ESTs | 0.29 | 1.94 | downregulate stage |
| 430778 | D90337 | Hs.247916 | natriuretic peptide precursor C | 0.14 | 4.48 | downregulate stage |
| 430998 | AF128847 | Hs.204038 | indolethylamine N-methyltransferase | 0.35 | 1.87 | downregulate stage |
| 432247 | AA531287 | Hs.105805 | ESTs | 0.21 | 3.99 | downregulate stage |
| 432689 | AB018320 | Hs.278626 | Arg/Abl-interacting protein ArgBP2 | 0.11 | 1.98 | downregulate stage |
| 432792 | AA448114 | Hs.278950 | protocadherin beta 1 | 0.22 | 2.93 | downregulate stage |
| 433142 | AL120697 | Hs.110640 | ESTs | 0.21 | 2.18 | downregulate stage |
| 433633 | AI880516 | Hs.84630 | ESTs | 0.34 | 2.67 | downregulate stage |
| 433688 | AA628467 | Hs.112572 | Homo sapiens cDNA FLJ14130 fis, clone MA | 0.35 | 2.27 | downregulate stage |
| 433826 | AA609938 | Hs.144492 | ESTs | 0.24 | 1.91 | downregulate stage |
| 434025 | AF114264 | Hs.216381 | Homo sapiens clone HH409 unknown mRNA | 0.07 | 3.46 | downregulate stage |
| 434160 | BE551196 | Hs.114275 | ESTs | 0.5 | 2 | downregulate stage |
| 434352 | AF129505 | Hs.86492 | small muscle protein, X-linked | 0.34 | 2.13 | downregulate stage |
| 434361 | AF129755 | Hs.117772 | ESTs | 0.03 | 31.3 | downregulate stage |
| 435731 | AA699581 | Hs.186811 | ESTs | 0.31 | 3.25 | downregulate stage |
| 435869 | AF255910 | Hs.54650 | vascular endothelial junction-associated | 0.21 | 3.73 | downregulate stage |
| 435978 | AF272899 | Hs.135118 | Homo sapiens PR-domain zinc finger prote | 0.35 | 2.25 | downregulate stage |
| 436359 | Z83806 | | gb: H. sapiens mRNA for axonemal dynein he | 0.24 | 3.28 | downregulate stage |
| 436638 | AI271945 | Hs.134984 | ESTs | 0.36 | 1.87 | downregulate stage |
| 436953 | AW959074 | Hs.23648 | Homo sapiens cDNA FLJ13097 fis, clone NT | 0.14 | 6.95 | downregulate stage |
| 437176 | AW176909 | Hs.42346 | calcineurin-binding protein calsarcin-1 | 0.32 | 2.19 | downregulate stage |
| 437233 | D81448 | Hs.153961 | ARP1 (actin-related protein 1, yeast) ho | 0.27 | 2.38 | downregulate stage |
| 438619 | AB032773 | Hs.6341 | TU12B1-TY protein | 0.19 | 2.69 | downregulate stage |
| 438666 | AW014493 | Hs.126727 | ESTs | 0.16 | 1.98 | downregulate stage |
| 439231 | AW581935 | Hs.141480 | ESTs | 0.1 | 3.9 | downregulate stage |
| 439973 | AI733308 | Hs.124663 | ESTs | 0.16 | 6.2 | downregulate stage |
| 440172 | AA868584 | Hs.126154 | ESTs | 0.24 | 2.34 | downregulate stage |
| 440274 | R24595 | Hs.7122 | scrapie responsive protein 1 | 0.11 | 3.02 | downregulate stage |
| 440687 | AL080222 | Hs.7358 | hypothetical protein FLJ13110 | 0.19 | 2.95 | downregulate stage |
| 440700 | AW952281 | Hs.296184 | ESTs, Highly similar to GB01_HUMAN GUANI | 0.13 | 2.69 | downregulate stage |
| 440737 | AI375167 | Hs.132221 | Homo sapiens cDNA FLJ12401 fis, clone MA | 0.5 | 2 | downregulate stage |
| 441805 | AA285136 | Hs.7974 | neuronal specific transcription factor D | 0.07 | 13.55 | downregulate stage |
| 441969 | AI733386 | Hs.129194 | ESTs, Weakly similar to ALU1_HUMAN ALU S | 0.38 | 1.86 | downregulate stage |
| 442109 | BE218975 | Hs.212395 | ESTs | 0.24 | 2.86 | downregulate stage |
| 442985 | AI025984 | Hs.55467 | ESTs | 0.19 | 2 | downregulate stage |
| 443060 | D78874 | Hs.8944 | procollagen C-endopeptidase enhancer 2 | 0.09 | 3.66 | downregulate stage |
| 443164 | AI038503 | Hs.55780 | ESTs, Weakly similar to ALU1_HUMAN ALU S | 0.2 | 1.86 | downregulate stage |
| 443476 | AW068594 | Hs.133878 | ESTs, Weakly similar to AF151889 1 CGI-1 | 0.11 | 2.79 | downregulate stage |
| 443604 | C03577 | Hs.9615 | myosin regulatory light chain 2, smooth | 0.24 | 3.41 | downregulate stage |
| 443790 | NM_003500 | Hs.9795 | acyl-Coenzyme A oxidase 2, branched chai | 0.28 | 3.6 | downregulate stage |
| 443932 | AW888222 | Hs.9973 | tensin | 0.32 | 2.57 | downregulate stage |
| 444195 | AB002351 | Hs.10587 | KIAA0353 protein | 0.19 | 4.04 | downregulate stage |
| 444484 | AK002126 | Hs.11260 | hypothetical protein FLJ11264 | 0.38 | 2.04 | downregulate stage |
| 444684 | AW044070 | Hs.147037 | ESTs | 0.36 | 2.25 | downregulate stage |
| 444793 | U89281 | Hs.11958 | oxidative 3 alpha hydroxysteroid dehydro | 0.29 | 2.19 | downregulate stage |
| 444938 | AW470690 | Hs.148814 | ESTs | 0.43 | 2.3 | downregulate stage |
| 445230 | U97018 | Hs.12451 | echinoderm microtubule-associated protei | 0.13 | 2.64 | downregulate stage |
| 445235 | AI564022 | Hs.138207 | ESTs | 0.13 | 2.4 | downregulate stage |
| 445621 | AI733818 | Hs.145549 | ESTs | 0.25 | 1.91 | downregulate stage |
| 445687 | W80382 | Hs.149297 | ESTs | 0.2 | 3.5 | downregulate stage |
| 445850 | AI262049 | Hs.145560 | ESTs | 0.53 | 1.9 | downregulate stage |
| 446406 | AI553681 | Hs.25248 | ESTs | 0.07 | 3.25 | downregulate stage |
| 446500 | U78093 | Hs.15154 | sushi-repeat-containing protein, X chrom | 0.33 | 1.9 | downregulate stage |
| 447595 | AW379130 | Hs.18953 | phosphodiesterase 9A | 0.28 | 1.85 | downregulate stage |
| 447918 | AI129320 | Hs.16930 | ESTs | 0.29 | 2.46 | downregulate stage |
| 448076 | AJ133123 | Hs.20196 | adenylate cyclase 9 | 0.2 | 2.27 | downregulate stage |
| 448283 | AI340462 | Hs.182979 | ribosomal protein L12 | 0.53 | 1.9 | downregulate stage |
| 448303 | BE622468 | Hs.11924 | ESTs, Weakly similar to ALU1_HUMAN ALU S | 0.39 | 1.84 | downregulate stage |
| 448425 | AI500359 | Hs.233401 | ESTs | 0.16 | 1.97 | downregulate stage |
| 448429 | D17408 | Hs.21223 | calponin 1, basic, smooth muscle | 0.12 | 5.43 | downregulate stage |
| 448555 | AI536697 | Hs.159863 | ESTs | 0.32 | 2.86 | downregulate stage |
| 448901 | AK001021 | Hs.22505 | hypothetical protein FLJ10159 | 0.17 | 2.66 | downregulate stage |
| 448999 | AF179274 | Hs.22791 | transmembrane protein with EGF-like and | 0.24 | 1.86 | downregulate stage |
| 449226 | AB002365 | Hs.23311 | KIAA0367 protein | 0.1 | 4.96 | downregulate stage |
| 449238 | AA428229 | Hs.85524 | muscle-specific RING-finger protein homo | 0.14 | 2.53 | downregulate stage |
| 449422 | AA001373 | Hs.59821 | ESTs | 0.43 | 2.3 | downregulate stage |
| 449690 | AA002140 | Hs.33024 | ESTs | 0.5 | 2 | downregulate stage |
| 449874 | AA135688 | Hs.10083 | ESTs | 0.33 | 2.7 | downregulate stage |
| 449925 | AI342493 | Hs.24192 | Homo sapiens cDNA FLJ20767 fis, clone CO | 0.11 | 5.57 | downregulate stage |
| 450300 | AL041440 | Hs.58210 | ESTs | 0.41 | 2.13 | downregulate stage |
| 450578 | AI971773 | Hs.232268 | ESTs | 0.44 | 2.25 | downregulate stage |
| 450810 | BE207588 | Hs.25511 | transforming growth factor beta 1 induce | 0.51 | 1.86 | downregulate stage |
| 450831 | R37974 | Hs.25255 | ESTs | 0.23 | 1.96 | downregulate stage |
| 451331 | AK002039 | Hs.26243 | Homo sapiens cDNA FLJ11177 fis, clone PL | 0.37 | 2.18 | downregulate stage |
| 451533 | NM_004657 | Hs.26530 | serum deprivation response (phosphatidyl | 0.1 | 9.36 | downregulate stage |
| 451782 | AF233588 | Hs.27018 | Ris | 0.35 | 2.43 | downregulate stage |
| 451948 | AW452473 | Hs.211125 | ESTs | 0.43 | 1.88 | downregulate stage |
| 452422 | AA521416 | Hs.22701 | ESTs | 0.41 | 1.85 | downregulate stage |
| 452463 | R36452 | Hs.300817 | ESTs | 0.09 | 4.05 | downregulate stage |
| 452776 | AA194540 | Hs.13522 | ESTs | 0.36 | 2.16 | downregulate stage |
| 452814 | AI092790 | Hs.55016 | hypothetical protein FLJ21935 | 0.06 | 4.7 | downregulate stage |
| 453064 | R40334 | Hs.301395 | Homo sapiens cDNA: FLJ21204 fis, clone C | 0.07 | 4.47 | downregulate stage |
| 453351 | AI625721 | Hs.61814 | Homo sapiens cDNA: FLJ22750 fis, clone K | 0.33 | 3.05 | downregulate stage |
| 453355 | AW295374 | Hs.31412 | Homo sapiens cDNA FLJ11422 fis, clone HE | 0.03 | 7.14 | downregulate stage |
| 453359 | AA448787 | Hs.24872 | ESTs, Weakly similar to aortic carboxype | 0.4 | 1.92 | downregulate stage |
| 453464 | AI884911 | Hs.32989 | receptor (calcitonin) activity modifying | 0.24 | 3.29 | downregulate stage |
| 453500 | AI478427 | Hs.43125 | ESTs | 0.02 | 11.41 | downregulate stage |
| 453582 | AW854339 | Hs.33476 | hypothetical protein FLJ11937 | 0.39 | 2.04 | downregulate stage |
| 453586 | AA248089 | Hs.50841 | ESTs, Weakly similar to tuftelin [M. musc | 0.43 | 1.86 | downregulate stage |
| 453666 | AW015681 | Hs.135229 | ESTs, Moderately similar to AF107203 1 a | 0.28 | 2.42 | downregulate stage |
| 453698 | AA037615 | Hs.42746 | ESTs | 0.2 | 1.88 | downregulate stage |
| 453702 | AA037637 | Hs.42128 | ESTs | 0.32 | 2.42 | downregulate stage |
| 453725 | W28543 | | gb: 48c5 Human retina cDNA randomly prime | 0.2 | 2.06 | downregulate stage |
| 453950 | AA156998 | Hs.211568 | eukaryotic translation initiation factor | 0.07 | 7.86 | downregulate stage |
| 454078 | AA601518 | Hs.22209 | secreted modular calcium-binding protein | 0.16 | 2.49 | downregulate stage |
| 454471 | AW902125 | | gb: QV0-NN1022-120500-220-h12 NN1022 Homo | 0.41 | 2.45 | downregulate stage |
| 454637 | AW811613 | | gb: CM3-ST0157-300999-017-f06 ST0157 Homo | 0.18 | 2.2 | downregulate stage |
| 454750 | AW866285 | | gb: QV4-SN0024-080400-167-a09 SN0024 Homo | 0.49 | 2.05 | downregulate stage |
| 455073 | AW854829 | | gb: QV2-CT0261-201099-011-f01 CT0261 Homo | 0.27 | 2.09 | downregulate stage |
| 455485 | AA102287 | Hs.26756 | Homo sapiens cDNA: FLJ20896 fis, clone A | 0.32 | 2.07 | downregulate stage |
| 455611 | L06419 | Hs.75093 | procollagen-lysine, 2-oxoglutarate 5-dio | 0.15 | 2.87 | downregulate stage |
| 456100 | AI983981 | Hs.189114 | ESTs | 0.4 | 2.5 | downregulate stage |
| 456841 | AA875863 | Hs.152345 | poliovirus receptor-related 1 (herpesvir | 0.35 | 1.9 | downregulate stage |
| 457064 | AA776743 | Hs.191589 | ESTs | 0.17 | 2.34 | downregulate stage |
| 457108 | N74724 | Hs.108479 | ESTs | 0.48 | 2.1 | downregulate stage |
| 457506 | AF131757 | Hs.274533 | Homo sapiens clone 24926 mRNA sequence | 0.29 | 2.59 | downregulate stage |
| 457625 | T10073 | | gb: seq1293 b4HB3MA Cot8-HAP-Ft Homo sapi | 0.29 | 3.45 | downregulate stage |
| 458482 | AV648858 | Hs.29488 | ESTs | 0.26 | 2.17 | downregulate stage |
| 458622 | AA972412 | Hs.13755 | f-box and WD-40 domain protein 2 | 0.51 | 1.95 | downregulate stage |
| 458841 | W28965 | | gb: 54d10 Human retina cDNA randomly prim | 0.32 | 3.1 | downregulate stage |
| 459037 | AW439497 | Hs.290656 | EST | 0.43 | 2.35 | downregulate stage |
| 400762 | | | | 0.71 | 0.4 | early stage |
| 400937 | | | | 1.2 | 0.26 | early stage |
| 400977 | | | | 0.63 | 0.48 | early stage |
| 401024 | | | | 0.8 | 0.3 | early stage |
| 401048 | | | | 1.9 | 0.22 | early stage |
| 401537 | | | | 1.3 | 0.2 | early stage |
| 401619 | | | | 3.5 | 0.19 | early stage |
| 402089 | | | | 0.39 | 0.55 | early stage |
| 402176 | | | | 0.35 | 0.91 | early stage |
| 402407 | | | | 1 | 0.15 | early stage |
| 402430 | | | | 0.28 | 1.25 | early stage |
| 402435 | | | | 2.15 | 0.21 | early stage |
| 402522 | | | | 1.8 | 0.14 | early stage |
| 402546 | | | | 0.17 | 1.66 | early stage |
| 402604 | | | | 0.41 | 0.66 | early stage |
| 402716 | | | | 0.14 | 0.86 | early stage |
| 402846 | | | | 0.61 | 0.52 | early stage |
| 402922 | | | | 0.14 | 0.83 | early stage |
| 403567 | | | | 0.44 | 0.49 | early stage |
| 403590 | | | | 1 | 0.34 | early stage |
| 404336 | | | | 0.49 | 0.44 | early stage |
| 404345 | AA730407 | Hs.159156 | protocadherin 11 | 0.38 | 0.4 | early stage |
| 404501 | AW247252 | Hs.75514 | nucleoside phosphorylase | 0.32 | 0.8 | early stage |
| 404594 | | | | 0.37 | 0.91 | early stage |
| 404874 | | | | 1.87 | 0.26 | early stage |
| 404881 | | | | 0.36 | 0.5 | early stage |
| 404896 | NM_000429 | Hs.106845 | methionine adenosyltransferase I, alpha | 1 | 0.36 | early stage |
| 404999 | U58196 | Hs.296281 | interleukin enhancer binding factor 1 | 0.19 | 1.06 | early stage |
| 405071 | | | | 0.19 | 0.77 | early stage |
| 405308 | | | | 0.4 | 0.55 | early stage |
| 405463 | | | | 0.41 | 1 | early stage |
| 405580 | | | | 1.89 | 0.19 | early stage |
| 405600 | | | | 0.22 | 0.63 | early stage |
| 405720 | | | | 0.37 | 0.61 | early stage |
| 405863 | | | | 0.53 | 0.26 | early stage |
| 405867 | | | | 0.24 | 1.1 | early stage |
| 405920 | | | | 0.39 | 1.15 | early stage |
| 406036 | | | | 2.15 | 0.17 | early stage |
| 406243 | | | | 0.32 | 1.23 | early stage |
| 406367 | | | | 0.38 | 0.76 | early stage |
| 406834 | AI318680 | | gb: ta49g09.x1 NCI_CGAP_Lu25 Homo sapiens | 0.4 | 0.67 | early stage |
| 406881 | D16154 | | gb: Human gene for cytochrome P-450c 11, e | 0.14 | 1.55 | early stage |
| 407411 | AF060170 | | gb: Homo sapiens AS12 protein mRNA, parti | 0.39 | 0.69 | early stage |
| 407639 | AW205369 | Hs.252936 | ESTs | 0.61 | 0.34 | early stage |
| 408112 | AW451982 | Hs.248613 | ESTs | 0.2 | 0.54 | early stage |
| 408732 | AL117490 | Hs.47225 | Homo sapiens mRNA; cDNA DKFZp434N211 (fr | 1 | 0.32 | early stage |
| 409103 | AF251237 | Hs.112208 | XAGE-1 protein | 0.33 | 1.03 | early stage |
| 409840 | AW502122 | | gb: UI-HF-BR0p-ajr-c-08-0-UI.r1 NIH_MGC_5 | 0.56 | 0.28 | early stage |
| 410128 | AW904599 | | gb: RC1-NN1063-260400-011-h05 NN1063 Homo | 1.26 | 0.37 | early stage |
| 411474 | AW848427 | | gb: IL3-CT0214-150200-075-H10 CT0214 Homo | 1 | 0.14 | early stage |
| 412564 | X83703 | Hs.74019 | cardiac ankyrin repeat protein | 0.36 | 0.44 | early stage |
| 413266 | BE300352 | | gb: 600944231F1 NIH_MGC_17 Homo sapiens c | 1.46 | 0.25 | early stage |
| 413341 | H78472 | Hs.191325 | ESTs, Weakly similar to cDNA EST yk414c9 | 0.41 | 0.48 | early stage |
| 414055 | AW818687 | Hs.5366 | Homo sapiens cDNA: FLJ21522 fis, clone C | 0.33 | 0.67 | early stage |
| 414170 | AA335996 | Hs.3743 | matrix metalloproteinase 24 (membrane-in | 1.15 | 0.21 | early stage |
| 414220 | BE298094 | | gb: 601118231F1 NIH_MGC_17 Homo sapiens c | 0.16 | 0.52 | early stage |
| 414276 | BE297862 | | gb: 601174780F1 NIH_MGC_17 Homo sapiens c | 1.75 | 0.2 | early stage |
| 414327 | BE408145 | Hs.185254 | ESTs, Moderately similar to NAC-1 protei | 0.1 | 0.99 | early stage |
| 414366 | BE549143 | | gb: 601076456F1 NIH_MGC_12 Homo sapiens c | 1 | 0.31 | early stage |
| 414376 | BE393856 | Hs.66915 | ESTs, Weakly similar to 16.7 Kd protein[ | 0.18 | 0.96 | early stage |
| 414555 | N98569 | Hs.76422 | phospholipase A2, group IIA (platelets, | 0.48 | 0.67 | early stage |
| 415199 | AA161125 | Hs.57893 | ESTs | 0.75 | 0.72 | early stage |
| 417304 | H15635 | | gb: ym27h06.r1 Soares infant brain 1NIB H | 0.6 | 0.58 | early stage |
| 417371 | N74613 | Hs.269149 | ESTs | 0.3 | 0.58 | early stage |
| 418133 | R43504 | Hs.6181 | ESTs | 1.28 | 0.29 | early stage |
| 419273 | BE271180 | Hs.293490 | ESTs | 0.54 | 0.28 | early stage |
| 419716 | AA953770 | | gb: on89e04.s1 Soares_NFL_T_GBC_S1 Homo s | 0.45 | 0.66 | early stage |
| 420390 | AA330047 | Hs.191187 | ESTs | 1.45 | 0.12 | early stage |
| 421745 | AF205849 | Hs.107740 | Kruppel-like factor 2 (lung) | 0.33 | 0.71 | early stage |
| 421813 | BE048255 | | gb: tz49b05.y1 NCI_CGAP_Brn52 Homo sapien | 0.52 | 0.67 | early stage |
| 422669 | H12402 | Hs.119122 | ribosomal protein L13a | 1 | 0.26 | early stage |
| 422743 | BE304678 | Hs.119598 | ribosomal protein L3 | 0.2 | 0.57 | early stage |
| 422760 | BE409561 | | gb: 601299865F1 NIH_MGC_21 Homo sapiens c | 0.41 | 0.64 | early stage |
| 422880 | AF228704 | Hs.121524 | glutathione reductase | 3.75 | 0.1 | early stage |
| 423457 | F08208 | Hs.155606 | paired mesoderm homeo box 1 | 0.55 | 0.54 | early stage |
| 425349 | AA425234 | Hs.79886 | ribose 5-phosphate isomerase A (ribose 5 | 1 | 0.21 | early stage |
| 425360 | BE547704 | | gb: 601076309F1 NIH_MGC_12 Homo sapiens c | 0.28 | 0.85 | early stage |
| 426356 | BE536836 | | gb: 601064837F1 NIH_MGC_10 Homo sapiens c | 0.31 | 0.69 | early stage |
| 426521 | AF161445 | Hs.170219 | hypothetical protein | 0.11 | 0.69 | early stage |
| 426670 | AA383047 | Hs.193718 | ESTs | 1 | 0.55 | early stage |
| 426699 | AA383337 | Hs.121269 | ESTs | 0.33 | 0.71 | early stage |
| 427827 | AA416577 | Hs.189105 | ESTs | 1.16 | 0.41 | early stage |
| 428651 | AF196478 | Hs.188401 | annexin A10 | 1.85 | 0.24 | early stage |
| 430727 | X75917 | Hs.2654 | MHC binding factor, beta | 0.78 | 0.46 | early stage |
| 430750 | AI650360 | Hs.100256 | ESTs | 2.15 | 0.17 | early stage |
| 430795 | AW971398 | | gb: EST383487 MAGE resequences, MAGL Homo | 1.95 | 0.21 | early stage |
| 431900 | AW972048 | Hs.192534 | ESTs | 0.36 | 0.73 | early stage |
| 432728 | NM_006979 | Hs.278721 | HLA class II region expressed gene KE4 | 1.8 | 0.17 | early stage |
| 432791 | NM_014554 | Hs.278949 | sentrin/SUMO-specific protease | 2.8 | 0.15 | early stage |
| 433404 | T32982 | Hs.102720 | ESTs | 2.2 | 0.13 | early stage |
| 433782 | AF090945 | | gb: Homo sapiens clone HQ0670 | 3.3 | 0.15 | early stage |
| 433877 | BE146567 | Hs.257475 | ESTs | 0.65 | 0.7 | early stage |
| 434483 | AA223646 | Hs.57222 | nurim (nuclear envelope membrane protein | 0.38 | 0.49 | early stage |
| 435752 | AF230801 | Hs.125180 | growth hormone receptor | 0.52 | 0.4 | early stage |
| 436178 | BE152396 | Hs.21590 | Homo sapiens HSPC304 mRNA, partial cds | 1.65 | 0.14 | early stage |
| 436391 | AJ227892 | Hs.146274 | ESTs | 1.35 | 0.16 | early stage |
| 436602 | AI793222 | Hs.166817 | ESTs | 0.17 | 1.46 | early stage |
| 436777 | AA731199 | Hs.293130 | ESTs | 1 | 0.2 | early stage |
| 436813 | AW975714 | Hs.129004 | ESTs | 0.19 | 1.45 | early stage |
| 436869 | NM_014867 | Hs.297661 | Homo sapiens YAC clone 377A1 unknown mRN | 0.96 | 0.2 | early stage |
| 437169 | AA309612 | Hs.118797 | ubiquitin-conjugating enzyme E2D 3 (homo | 0.07 | 1.8 | early stage |
| 438649 | AA813136 | Hs.143014 | ESTs | 1.38 | 0.19 | early stage |
| 438681 | AW384815 | Hs.149208 | KIAA1555 protein | 0.25 | 0.54 | early stage |
| 438802 | AA825976 | Hs.136954 | ESTs | 1.8 | 0.14 | early stage |
| 438887 | R68857 | Hs.265499 | ESTs | 1.05 | 0.32 | early stage |
| 440128 | AA962623 | Hs.189144 | ESTs, Weakly similar to NPT2_HUMAN RENAL | 1 | 0.19 | early stage |
| 440397 | AA884448 | Hs.157239 | ESTs | 0.59 | 0.38 | early stage |
| 440509 | BE410132 | Hs.134202 | ESTs, Weakly similar to B41182 collagen | 0.26 | 0.9 | early stage |
| 440605 | Z40094 | Hs.185698 | ESTs | 0.51 | 0.43 | early stage |
| 442238 | AW135374 | Hs.270949 | ESTs | 1 | 0.18 | early stage |
| 443258 | AF169301 | Hs.9098 | sulfate transporter 1 | 0.85 | 0.49 | early stage |
| 445739 | AW136354 | Hs.145303 | ESTs | 0.88 | 0.4 | early stage |
| 447306 | AI373163 | Hs.170333 | ESTs | 0.15 | 0.8 | early stage |
| 447346 | AI525135 | Hs.210507 | ESTs | 1.35 | 0.27 | early stage |
| 448265 | N46272 | Hs.26812 | ESTs | 0.47 | 0.26 | early stage |
| 448678 | AI560776 | Hs.199854 | ESTs | 0.19 | 0.68 | early stage |
| 448778 | AF074913 | | gb: Homo sapiens transcription factor Pax | 0.57 | 0.53 | early stage |
| 448871 | BE616709 | | gb: 601279937F1 NIH_MGC_39 Homo sapiens c | 0.26 | 0.94 | early stage |
| 449180 | AI633836 | Hs.195649 | ESTs | 0.46 | 0.45 | early stage |
| 449213 | BE616861 | | gb: 601279056F1 NIH_MGC_39 Homo sapiens c | 0.73 | 0.56 | early stage |
| 449231 | BE410360 | | gb: 601302340F1 NIH_MGC_21 Homo sapiens c | 0.27 | 0.76 | early stage |
| 449450 | AL039852 | Hs.256990 | ESTs, Moderately similar to ALU7_HUMAN A | 1 | 0.26 | early stage |
| 449815 | AI671000 | Hs.199739 | ESTs | 1.2 | 0.15 | early stage |
| 450972 | AW967906 | Hs.194617 | ESTs | 0.28 | 0.83 | early stage |
| 451236 | AI767406 | Hs.207026 | ESTs, Weakly similar B56205 transcrip | 0.35 | 0.77 | early stage |
| 451283 | H83979 | | gb: ys93d11.r1 Soares retina N2b5HR Homo | 1 | 0.23 | early stage |
| 451375 | AI792066 | Hs.283902 | Homo sapiens BAC clone RP11-481J13 from | 0.16 | 1.37 | early stage |
| 452530 | AI905518 | | gb: RC-BT091-210199-098 BT091 Homo sapien | 1.35 | 0.21 | early stage |
| 452550 | AA026735 | | gb: ze93d05.r1 Soares_fetal_heart_NbHH19W | 0.44 | 0.6 | early stage |
| 454121 | AW090524 | Hs.244967 | ESTs | 2.85 | 0.17 | early stage |
| 454554 | AW847505 | | gb: RC0-CT0210-280999-021-c10 CT0210 Homo | 0.36 | 0.5 | early stage |
| 454697 | AW813728 | Hs.15036 | ESTs, Highly similar to AF161358 1 HSPC0 | 0.43 | 0.6 | early stage |
| 454851 | AW835127 | | gb: RC4-LT0011-100100-012-c07 LT0011 Homo | 0.77 | 0.32 | early stage |
| 455040 | AW852286 | | gb: QV0-CT0225-100400-187-d08 CT0225 Homo | 0.26 | 0.52 | early stage |
| 455225 | AW996689 | | gb: QV3-BN0046-150400-151-g09 BN0046 Homo | 1.7 | 0.18 | early stage |
| 455970 | AI733857 | Hs.71483 | ESTs | 0.66 | 0.45 | early stage |
| 456235 | AA203637 | | gb: zx58b12.r1 Soares_fetal_liver_spleen_{—} | 0.64 | 0.43 | early stage |
| 456450 | AJ000098 | Hs.94210 | eyes absent (Drosophila) homolog 1 | 0.38 | 0.76 | early stage |
| 456526 | AA782222 | Hs.192008 | ESTs | 0.63 | 0.43 | early stage |
| 456855 | AF035528 | Hs.153863 | MAD (mothers against decapentaplegic, Dr | 0.49 | 0.46 | early stage |
| 456983 | AI081687 | Hs.170225 | thymopoietin | 0.27 | 0.75 | early stage |
| 457089 | AA416556 | Hs.98234 | ESTs | 0.34 | 0.48 | early stage |
| 458198 | AI286100 | Hs.192739 | ESTs | 0.47 | 0.48 | early stage |
| 458425 | AI084057 | Hs.301149 | ESTs | 0.4 | 0.37 | early stage |
| 458660 | AI299739 | Hs.99601 | Homo sapiens cDNA FLJ12553 fis, clone NT | 0.79 | 0.68 | early stage |
| 458703 | AW749121 | Hs.282901 | ESTs | 1 | 0.23 | early stage |
| 458767 | T97083 | Hs.148355 | ESTs | 1 | 0.17 | early stage |
| 459399 | BE407712 | | gb: 601299745F1 NIH_MGC_21 Homo sapiens c | 0.68 | 0.56 | early stage |
| 400860 | | | | 4.9 | 0.08 | late stage |
| 408190 | AB032963 | Hs.43577 | ATPase, Class I, type 8B, member 2 | 0.58 | 0.84 | late stage |
| 408558 | AW015759 | Hs.235709 | ESTs | 1.26 | 0.45 | late stage |
| 410077 | AF097645 | Hs.58570 | deleted in cancer 1; RNA helicase HDB/DI | 6.2 | 0.12 | late stage |
| 410295 | AA741357 | Hs.62041 | nidogen (enactin) | 0.77 | 0.86 | late stage |
| 410310 | J02931 | Hs.62192 | coagulation factor III (thromboplastin, | 1.45 | 0.27 | late stage |
| 410614 | AI091195 | Hs.65029 | growth arrest-specific 1 | 0.4 | 1.12 | late stage |
| 410867 | X63556 | Hs.750 | fibrillin 1 (Marfan syndrome) | 0.71 | 1.07 | late stage |
| 411573 | AB029000 | Hs.70823 | KIAA1077 protein | 3.64 | 0.19 | late stage |
| 412116 | AW402166 | Hs.784 | Epstein-Barr virus induced gene 2 (lymph | 5.18 | 0.13 | late stage |
| 412178 | AW898526 | | gb: RC6-NN0072-040500-011-E05 NN0072 Homo | 7.55 | 0.08 | late stage |
| 412429 | AV650262 | Hs.75765 | GRO2 oncogene | 3.37 | 0.15 | late stage |
| 412652 | AI801777 | Hs.6774 | ESTs | 0.49 | 1.24 | late stage |
| 412828 | AL133396 | Hs.74621 | prion protein (p27-30) (Creutzfeld-Jakob | 3.6 | 0.11 | late stage |
| 414020 | NM_002984 | Hs.75703 | small inducible cytokine A4 (homologous | 4.62 | 0.14 | late stage |
| 414183 | AW957446 | Hs.301711 | ESTs | 3.18 | 0.16 | late stage |
| 414359 | M62194 | Hs.75929 | cadherin 11, type 2, OB-cadherin (osteob | 0.81 | 0.73 | late stage |
| 414476 | AA301867 | Hs.76224 | EGF-containing fibulin-like extracellula | 0.37 | 0.99 | late stage |
| 414504 | AW069181 | Hs.293523 | ESTs, Weakly similar to transformation-r | 0.97 | 0.65 | late stage |
| 414812 | X72755 | Hs.77367 | monokine induced by gamma interferon | 3.84 | 0.1 | late stage |
| 415116 | AA160363 | Hs.269956 | ESTs | 7.45 | 0.07 | late stage |
| 415714 | NM_002290 | Hs.78672 | laminin, alpha 4 | 0.49 | 1.39 | late stage |
| 415822 | D59243 | | gb: HUM526E07B Clontech human placenta po | 8.15 | 0.09 | late stage |
| 415994 | NM_002923 | Hs.78944 | regulator of G-protein signalling 2, 24 k | 0.48 | 1.46 | late stage |
| 417059 | AL037672 | Hs.81071 | extracellular matrix protein 1 | 1.52 | 0.44 | late stage |
| 417259 | AW903838 | Hs.81800 | chondroitin sulfate proteoglycan 2 (vers | 2 | 0.39 | late stage |
| 417733 | AL048678 | Hs.82503 | syntaphilin | 0.2 | 2.67 | late stage |
| 417771 | AA804698 | Hs.82547 | retinoic acid receptor responder (tazaro | 4.56 | 0.12 | late stage |
| 417849 | AW291587 | Hs.82733 | nidogen 2 | 1.81 | 0.38 | late stage |
| 418005 | AI186220 | Hs.83164 | collagen, type XV, alpha 1 | 0.97 | 0.74 | late stage |
| 418283 | S79895 | Hs.83942 | cathepsin K (pycnodysostosis) | 1.21 | 0.56 | late stage |
| 418875 | W19971 | Hs.233459 | ESTs | 2.63 | 0.33 | late stage |
| 419490 | NM_006144 | Hs.90708 | granzyme A (granzyme 1, cytotoxic T-lymp | 7.65 | 0.07 | late stage |
| 419925 | AA159850 | Hs.93765 | lipoma HMGIC fusion partner | 0.91 | 0.82 | late stage |
| 420411 | AI581085 | Hs.24678 | ESTs | 7.3 | 0.1 | late stage |
| 420943 | AI718702 | Hs.105341 | ESTs | 7.05 | 0.07 | late stage |
| 421116 | T19132 | Hs.101850 | retinol-binding protein 1, cellular | 0.99 | 0.42 | late stage |
| 421684 | BE281591 | Hs.106768 | hypothetical protein FLJ10511 | 8.1 | 0.08 | late stage |
| 421786 | AI188653 | Hs.21351 | ESTs | 8.15 | 0.08 | late stage |
| 422414 | AW875237 | Hs.13701 | ESTs | 1.05 | 0.69 | late stage |
| 422550 | BE297626 | Hs.296049 | microfibrillar-associated protein 4 | 0.28 | 1.53 | late stage |
| 422790 | AA809875 | Hs.25933 | ESTs | 2.59 | 0.28 | late stage |
| 423057 | AW961597 | Hs.130816 | ESTs | 7.55 | 0.08 | late stage |
| 423720 | AL044191 | Hs.23388 | Homo sapiens cDNA: FLJ21310 fis, clone C | 1.24 | 0.61 | late stage |
| 423905 | AW579960 | Hs.135150 | lung type-I cell membrane-associated gly | 2.12 | 0.24 | late stage |
| 423915 | AF039018 | Hs.135281 | alpha-actinin-2-associated LIM protein | 0.29 | 2.45 | late stage |
| 423961 | D13666 | Hs.136348 | osteoblast specific factor 2 (fasciclin | 4.47 | 0.17 | late stage |
| 424247 | X14008 | Hs.234734 | lysozyme (renal amyloidosis) | 2.44 | 0.26 | late stage |
| 424839 | AA740632 | Hs.120850 | ESTs | 2.74 | 0.23 | late stage |
| 426780 | BE242284 | Hs.172199 | adenylate cyclase 7 | 8.55 | 0.09 | late stage |
| 426974 | AB002298 | Hs.173035 | KIAA0300 protein | 1.56 | 0.36 | late stage |
| 427055 | AI301740 | Hs.173381 | dihydropyrimidinase-like 2 | 0.72 | 1 | late stage |
| 427882 | AA640987 | Hs.193767 | ESTs | 2.25 | 0.29 | late stage |
| 428065 | AI634046 | Hs.157313 | ESTs | 6.19 | 0.1 | late stage |
| 428147 | AW629965 | Hs.234983 | ESTs | 8.42 | 0.08 | late stage |
| 428585 | AB007863 | Hs.185140 | KIAA0403 protein | 6.85 | 0.08 | late stage |
| 428825 | AI084336 | Hs.128783 | ESTs | 0.9 | 0.8 | late stage |
| 429490 | AI971131 | Hs.293684 | ESTs, Weakly similar to alternatively sp | 1.59 | 0.39 | late stage |
| 429500 | X78565 | Hs.289114 | hexabrachion (tenascin C, cytotactin) | 0.77 | 0.49 | late stage |
| 431103 | M57399 | Hs.44 | pleiotrophin (heparin binding growth fac | 0.92 | 0.3 | late stage |
| 431319 | AA873350 | | gb: oh64h02.s1 NCI_CGAP_Kid5 Homo sapiens | 1.36 | 0.44 | late stage |
| 431583 | AL042613 | Hs.262476 | S-adenosylmethionine decarboxylase 1 | 4.69 | 0.17 | late stage |
| 432314 | AA533447 | Hs.285173 | ESTs | 1.75 | 0.31 | late stage |
| 432331 | W37862 | Hs.274368 | Homo sapiens mRNA; cDNA DKFZp586I1524 (f | 0.35 | 1.58 | late stage |
| 432559 | AW452948 | Hs.257631 | ESTs | 1.37 | 0.49 | late stage |
| 433470 | AW960564 | Hs.3337 | transmembrane 4 superfamily member 1 | 2.58 | 0.24 | late stage |
| 433586 | T85301 | | gb: yd78d06.s1 Soares fetal liver spleen | 5.06 | 0.11 | late stage |
| 436428 | AW246900 | Hs.283712 | hypothetical protein | 8.25 | 0.09 | late stage |
| 436729 | BE621807 | Hs.3337 | transmembrane 4 superfamily member 1 | 1.6 | 0.26 | late stage |
| 438873 | AI302471 | Hs.124292 | Homo sapiens cDNA: FLJ23123 fis, clone L | 8.15 | 0.08 | late stage |
| 439584 | AA838114 | Hs.221612 | ESTs | 8.6 | 0.09 | late stage |
| 439653 | AW021103 | Hs.6631 | hypothetical protein FLJ20373 | 2.21 | 0.27 | late stage |
| 440524 | R71264 | Hs.16798 | ESTs | 3.44 | 0.21 | late stage |
| 440624 | AF017987 | Hs.7306 | secreted frizzled-related protein 1 | 0.42 | 0.63 | late stage |
| 441976 | AA428403 | Hs.106131 | ESTs | 8.5 | 0.09 | late stage |
| 442739 | NM_007274 | Hs.8679 | cytosolic acyl coenzyme A thioester hydr | 7.95 | 0.06 | late stage |
| 443852 | AI679966 | Hs.150603 | ESTs | 6.84 | 0.12 | late stage |
| 443896 | AI680242 | Hs.271687 | Homo sapiens cDNA FLJ13527 fis, clone PL | 7.95 | 0.08 | late stage |
| 444212 | AW503976 | Hs.10649 | basement membrane-induced gene | 2.31 | 0.28 | late stage |
| 444331 | AW193342 | Hs.24144 | ESTs | 0.32 | 1.64 | late stage |
| 445142 | AW978484 | Hs.93842 | Homo sapiens cDNA: FLJ22554 fis, clone H | 2.52 | 0.24 | late stage |
| 445701 | AF055581 | Hs.13131 | lymphocyte adaptor protein | 1.43 | 0.47 | late stage |
| 446584 | U53445 | Hs.15432 | downregulated in ovarian cancer 1 | 0.54 | 1.39 | late stage |
| 447526 | AL048753 | Hs.340 | small inducible cytokine A2 (monocyte ch | 1.43 | 0.43 | late stage |
| 447744 | AA313230 | Hs.19413 | S100 calcium-binding protein A12 (calgra | 1.35 | 0.26 | late stage |
| 447818 | W79940 | Hs.21906 | ESTs | 6.63 | 0.13 | late stage |
| 449567 | AI990790 | Hs.188614 | ESTs | 4.7 | 0.13 | late stage |
| 450455 | AL117424 | Hs.25035 | chloride intracellular channel 4 | 0.64 | 1.31 | late stage |
| 452239 | AW379378 | Hs.170121 | protein tyrosine phosphatase, receptor t | 0.59 | 1.18 | late stage |
| 452698 | NM_001295 | Hs.301921 | ESTs | 2.31 | 0.26 | late stage |
| 453212 | H15416 | Hs.21865 | ESTs | 2.51 | 0.26 | late stage |
| 455510 | AA422029 | Hs.143640 | ESTs, Weakly similar to hyperpolarizatio | 8.6 | 0.06 | late stage |
| 400775 | R58624 | Hs.2186 | eukaryotic translation elongation factor | 1 | 1 | T2-T4 grade 3 papilloma marker |
| 401508 | | | | 1 | 1 | T2-T4 grade 3 papilloma marker |
| 403092 | | | | 1 | 1 | T2-T4 grade 3 papilloma marker |
| 404232 | | | | 1 | 1 | T2-T4 grade 3 papilloma marker |
| 407020 | U49973 | | gb: Human Tigger1 transposable element, c | 1 | 1 | T2-T4 grade 3 papilloma marker |
| 407345 | AI053836 | Hs.169365 | ESTs, Weakly similar to ALU1_HUMAN ALU S | 1 | 1 | T2-T4 grade 3 papilloma marker |
| 407420 | AF084362 | | gb: Homo sapiens lipoate-protein ligase B | 1 | 1 | T2-T4 grade 3 papilloma marker |
| 407577 | AW131324 | Hs.246759 | ESTs, Weakly similar to KIAA1074 protein | 1 | 1 | T2-T4 grade 3 papilloma marker |
| 407666 | AF071107 | Hs.37501 | MAD (mothers against decapentaplegic, Dr | 1 | 1 | T2-T4 grade 3 papilloma marker |
| 407916 | L09234 | Hs.603 | ATPase, H+ transporting, lysosomal (vacu | 1 | 1 | T2-T4 grade 3 papilloma marker |
| 407936 | AW118147 | Hs.270935 | ESTs | 1 | 1 | T2-T4 grade 3 papilloma marker |
| 408186 | AW168847 | Hs.250156 | ESTs | 1 | 1 | T2-T4 grade 3 papilloma marker |
| 408950 | AA707814 | Hs.7396 | ESTs | 1 | 1 | T2-T4 grade 3 papilloma marker |
| 409038 | T97490 | Hs.50002 | small inducible cytokine subfamily A (Cy | 1.2 | 0.12 | T2-T4 grade 3 papilloma marker |
| 409045 | AA635062 | Hs.50094 | Homo sapiens mRNA; cDNA DKFZp434O0515 (f | 1 | 1 | T2-T4 grade 3 papilloma marker |
| 409196 | NM_001874 | Hs.169765 | carboxypeptidase M | 1 | 1 | T2-T4 grade 3 papilloma marker |
| 409281 | AA069998 | | gb: zm67b03.r1 Stratagene neuroepithelium | 1 | 1 | T2-T4 grade 3 papilloma marker |
| 410010 | AW572853 | Hs.257683 | ESTs, Weakly similar to ALU3_HUMAN ALU S | 1 | 0.5 | T2-T4 grade 3 papilloma marker |
| 410157 | AW593277 | Hs.225056 | ESTs | 1 | 0.69 | T2-T4 grade 3 papilloma marker |
| 411112 | AW818158 | | gb: CM1-ST0277-161299-070-g07 ST0277 Homo | 1 | 1 | T2-T4 grade 3 papilloma marker |
| 411336 | AW837675 | | gb: QV2-LT0039-260300-107-b04 LT0039 Homo | 1 | 1 | T2-T4 grade 3 papilloma marker |
| 412051 | T15872 | Hs.268713 | ESTs, Weakly similar to hypothetical pro | 1 | 1 | T2-T4 grade 3 papilloma marker |
| 413485 | N52628 | | gb: yv37g11.s1 Soares fetal liver spleen | 1 | 1 | T2-T4 grade 3 papilloma marker |
| 413574 | BE149158 | Hs.129998 | Homo sapiens cDNA FLJ14267 fis, clone PL | 1 | 1 | T2-T4 grade 3 papilloma marker |
| 413782 | BE546104 | | gb: 601072642F1 NIH_MGC_12 Homo sapiens c | 1 | 1 | T2-T4 grade 3 papilloma marker |
| 414749 | H94622 | Hs.193358 | ESTs, Moderately similar to diabetes mel | 1 | 1 | T2-T4 grade 3 papilloma marker |
| 415293 | R49462 | Hs.106541 | ESTs | 1 | 1 | T2-T4 grade 3 papilloma marker |
| 415442 | F12963 | Hs.7045 | GL004 protein | 1 | 1 | T2-T4 grade 3 papilloma marker |
| 416255 | T87587 | Hs.272082 | ESTs | 1 | 1 | T2-T4 grade 3 papilloma marker |
| 417047 | AA192640 | Hs.1526 | ATPase, Ca++ transporting, cardiac muscl | 1 | 1 | T2-T4 grade 3 papilloma marker |
| 417181 | L10123 | Hs.1071 | surfactant protein A binding protein | 1 | 1 | T2-T4 grade 3 papilloma marker |
| 417367 | N73877 | Hs.171815 | ESTs | 1 | 1 | T2-T4 grade 3 papilloma marker |
| 419721 | NM_001650 | Hs.288650 | aquaporin 4 | 1 | 1 | T2-T4 grade 3 papilloma marker |
| 420294 | AA808259 | Hs.196716 | ESTs | 1 | 0.65 | T2-T4 grade 3 papilloma marker |
| 423589 | AA328082 | Hs.209569 | ESTs, Weakly similar to thrombospondin t | 1 | 1 | T2-T4 grade 3 papilloma marker |
| 424549 | AI873205 | Hs.183114 | Homo sapiens cDNA FLJ14236 fis, clone NT | 1 | 1 | T2-T4 grade 3 papilloma marker |
| 425458 | H89317 | Hs.182889 | ESTs | 1 | 1 | T2-T4 grade 3 papilloma marker |
| 426475 | AL134728 | | gb: DKFZp547A1890_r1 547 (synonym: hfbr1) | 1 | 1 | T2-T4 grade 3 papilloma marker |
| 429453 | AA453195 | Hs.124222 | ESTs | 1 | 1 | T2-T4 grade 3 papilloma marker |
| 431200 | AF044923 | Hs.250752 | hook1 protein | 1 | 1 | T2-T4 grade 3 papilloma marker |
| 431938 | AA938471 | Hs.115242 | developmentally regulated GTP-binding pr | 1 | 1 | T2-T4 grade 3 papilloma marker |
| 431944 | AI360891 | Hs.143619 | ESTs | 1 | 1 | T2-T4 grade 3 papilloma marker |
| 432021 | AA524470 | Hs.58753 | ESTs | 1 | 1 | T2-T4 grade 3 papilloma marker |
| 432205 | AI806583 | Hs.125291 | ESTs | 1 | 0.31 | T2-T4 grade 3 papilloma marker |
| 432527 | AW975028 | Hs.102754 | ESTs | 1 | 1 | T2-T4 grade 3 papilloma marker |
| 434069 | AF116651 | Hs.283058 | hypothetical protein PRO0800 | 1 | 0.41 | T2-T4 grade 3 papilloma marker |
| 435278 | AW994242 | Hs.173495 | ESTs | 1 | 1 | T2-T4 grade 3 papilloma marker |
| 435965 | AI034368 | Hs.132650 | ESTs | 1 | 0.36 | T2-T4 grade 3 papilloma marker |
| 436227 | AA706937 | Hs.120802 | ESTs, Moderately similar to A26641 Na+/K | 1 | 1 | T2-T4 grade 3 papilloma marker |
| 436635 | AW104325 | Hs.272093 | ESTs, Weakly similar to STK2_HUMAN SERIN | 1 | 0.74 | T2-T4 grade 3 papilloma marker |
| 436640 | AA724411 | Hs.156065 | ESTs | 1 | 1 | T2-T4 grade 3 papilloma marker |
| 436884 | BE046657 | | gb: hn42e02.x1 NCI_CGAP_RDF2 Homo sapiens | 1 | 1 | T2-T4 grade 3 papilloma marker |
| 437251 | AW976082 | | gb: EST388191 MAGE resequences, MAGN Homo | 1 | 1 | T2-T4 grade 3 papilloma marker |
| 437348 | AA749149 | Hs.163114 | ESTs | 1 | 1 | T2-T4 grade 3 papilloma marker |
| 437769 | AA767853 | Hs.122895 | ESTs | 1 | 1 | T2-T4 grade 3 papilloma marker |
| 437771 | AA811071 | Hs.123349 | ESTs | 1 | 1 | T2-T4 grade 3 papilloma marker |
| 438347 | AA909686 | Hs.293397 | ESTs | 1 | 1 | T2-T4 grade 3 papilloma marker |
| 439171 | AA831133 | Hs.294128 | ESTs | 1 | 0.95 | T2-T4 grade 3 papilloma marker |
| 439914 | AA854066 | Hs.145394 | ESTs | 1 | 1 | T2-T4 grade 3 papilloma marker |
| 440399 | AI215527 | Hs.125589 | ESTs | 1 | 1 | T2-T4 grade 3 papilloma marker |
| 440972 | BE044588 | Hs.276158 | ESTs | 1 | 1 | T2-T4 grade 3 papilloma marker |
| 442004 | AA973568 | Hs.128317 | ESTs | 1 | 1 | T2-T4 grade 3 papilloma marker |
| 442270 | BE565699 | Hs.62005 | ESTs | 1 | 1 | T2-T4 grade 3 papilloma marker |
| 443413 | AI056457 | Hs.221642 | ESTs | 1 | 1 | T2-T4 grade 3 papilloma marker |
| 443927 | AW016726 | Hs.134860 | ESTs | 1 | 1 | T2-T4 grade 3 papilloma marker |
| 445442 | N20392 | Hs.42846 | ESTs | 1 | 1 | T2-T4 grade 3 papilloma marker |
| 445611 | AW418497 | Hs.145583 | ESTs | 1 | 0.49 | T2-T4 grade 3 papilloma marker |
| 445888 | AF070564 | Hs.13415 | Homo sapiens clone 24571 mRNA sequence | 1 | 1 | T2-T4 grade 3 papilloma marker |
| 446552 | AW470827 | Hs.156241 | ESTs | 1 | 1 | T2-T4 grade 3 papilloma marker |
| 447399 | AI815401 | Hs.251967 | Homo sapiens clone 785627 unknown mRNA | 2.9 | 0.14 | T2-T4 grade 3 papilloma marker |
| 449111 | T83109 | Hs.196180 | ESTs | 1 | 1 | T2-T4 grade 3 papilloma marker |
| 449232 | AW192780 | Hs.196080 | ESTs | 1 | 0.8 | T2-T4 grade 3 papilloma marker |
| 451373 | AI792030 | | gb: os03e11.y5 NCI_CGAP_Lu5 Homo sapiens | 1 | 1 | T2-T4 grade 3 papilloma marker |
| 452453 | AI902519 | | gb: QV-BT009-101198-051 BT009 Homo sapien | 1 | 1 | T2-T4 grade 3 papilloma marker |
| 452534 | AW083022 | Hs.149425 | Homo sapiens cDNA FLJ11980 fis, clone HE | 1 | 0.67 | T2-T4 grade 3 papilloma marker |
| 452536 | BE063380 | | gb: PM0-BT0275-291099-002-g 10 BT0275 Homo | 1.65 | 0.26 | T2-T4 grade 3 papilloma marker |
| 452640 | AA027115 | Hs.100206 | ESTs, Weakly similar to AAAD_HUMAN ARYLA | 1 | 1 | T2-T4 grade 3 papilloma marker |
| 452645 | AI911325 | Hs.212049 | EST | 1 | 1 | T2-T4 grade 3 papilloma marker |
| 453102 | NM_007197 | Hs.31664 | frizzled (Drosophila) homolog 10 | 1 | 1 | T2-T4 grade 3 papilloma marker |
| 453472 | AL037925 | | gb: DKFZp564M037_r1 564 (synonym: hfbr2) | 1 | 0.8 | T2-T4 grade 3 papilloma marker |
| 453609 | AL045301 | Hs.13427 | ESTs | 1 | 1 | T2-T4 grade 3 papilloma marker |
| 453677 | AL079389 | | gb: DKFZp434E2116_r1 434 (synonym: htes3) | 1 | 0.77 | T2-T4 grade 3 papilloma marker |
| 453704 | R41806 | Hs.100884 | ESTs | 1 | 1 | T2-T4 grade 3 papilloma marker |
| 455267 | AW880861 | | gb: QV0-OT0033-070300-152-c12 OT0033 Homo | 1 | 1 | T2-T4 grade 3 papilloma marker |
| 455880 | BE153208 | | gb: PM0-HT0335-050400-007-F10 HT0335 Homo | 1 | 1 | T2-T4 grade 3 papilloma marker |
| 456520 | AW835416 | Hs.29417 | HCF-binding transcription factor Zhangfe | 1 | 1 | T2-T4 grade 3 papilloma marker |
| 456763 | AJ271351 | Hs.128180 | B-cell translocation gene 4 | 1 | 0.71 | T2-T4 grade 3 papilloma marker |
| 456912 | AI458843 | Hs.158112 | protein tyrosine phosphatase, receptor t | 1 | 1 | T2-T4 grade 3 papilloma marker |
| 457018 | AA761820 | Hs.250965 | ESTs | 1 | 1 | T2-T4 grade 3 papilloma marker |
| 457323 | AW967813 | Hs.201064 | ESTs | 1 | 1 | T2-T4 grade 3 papilloma marker |
| 457339 | AW971949 | Hs.291252 | ESTs | 1 | 1 | T2-T4 grade 3 papilloma marker |
| 457340 | AA492071 | | gb: ne97b04.s1 NCI_CGAP_Kid1 Homo sapiens | 1 | 1 | T2-T4 grade 3 papilloma marker |
| 457507 | AW300248 | Hs.181693 | ESTs | 1 | 1 | T2-T4 grade 3 papilloma marker |
| 458106 | AF086561 | Hs.37 | acetyl-Coenzyme A acetyltransferase 1 (a | 1 | 1 | T2-T4 grade 3 papilloma marker |
| 458624 | AI362790 | Hs.181801 | ESTs | 1 | 0.34 | T2-T4 grade 3 papilloma marker |
| 459396 | AI907536 | Hs.103869 | ESTs | 1 | 1 | T2-T4 grade 3 papilloma marker |
| 401002 | | | | 1 | 1 | T2-T4 grade 3 solid tumor marker |
| 401866 | | | | 1.35 | 0.14 | T2-T4 grade 3 solid tumor marker |
| 403615 | | | | 1 | 1 | T2-T4 grade 3 solid tumor marker |
| 403776 | | | | 1 | 1 | T2-T4 grade 3 solid tumor marker |
| 404113 | | | | 1 | 0.43 | T2-T4 grade 3 solid tumor marker |
| 404488 | | | | 1 | 0.17 | T2-T4 grade 3 solid tumor marker |
| 404653 | | | | 1 | 1 | T2-T4 grade 3 solid tumor marker |
| 406076 | AL390179 | Hs.137011 | Homo sapiens mRNA; cDNA DKFZp547P134 (fr | 1 | 0.24 | T2-T4 grade 3 solid tumor marker |
| 406471 | | | | 1 | 0.42 | T2-T4 grade 3 solid tumor marker |
| 406690 | M29540 | Hs.220529 | carcinoembryonic antigen-related cell ad | 2.75 | 0.05 | T2-T4 grade 3 solid tumor marker |
| 407624 | AW157431 | Hs.248941 | ESTs | 3.05 | 0.15 | T2-T4 grade 3 solid tumor marker |
| 409153 | W03754 | Hs.50813 | hypothetical protein FLJ20022 | 3.85 | 0.03 | T2-T4 grade 3 solid tumor marker |
| 409464 | X69115 | Hs.54488 | zinc finger protein 37a (KOX 21) | 1.15 | 0.29 | T2-T4 grade 3 solid tumor marker |
| 409731 | AA125985 | Hs.56145 | thymosin, beta, identified in neuroblast | 1 | 0.24 | T2-T4 grade 3 solid tumor marker |
| 410025 | BE220489 | Hs.113592 | ESTs | 1 | 0.3 | T2-T4 grade 3 solid tumor marker |
| 410589 | AW770768 | Hs.266717 | ESTs | 1 | 0.28 | T2-T4 grade 3 solid tumor marker |
| 411840 | AW866330 | | gb: QV4-SN0024-080400-167-e01 SN0024 Homo | 1.05 | 0.15 | T2-T4 grade 3 solid tumor marker |
| 412198 | AA937111 | Hs.69165 | ESTs | 1 | 0.26 | T2-T4 grade 3 solid tumor marker |
| 412305 | AW936369 | | gb: QV4-DT0021-301299-071-d07 DT0021 Homo | 1 | 0.53 | T2-T4 grade 3 solid tumor marker |
| 412753 | AI065016 | Hs.6390 | ESTs | 1 | 0.33 | T2-T4 grade 3 solid tumor marker |
| 413472 | BE242870 | Hs.75379 | solute carrier family 1 (glial high affi | 1 | 0.69 | T2-T4 grade 3 solid tumor marker |
| 413530 | AA130158 | Hs.19977 | ESTs, Moderately similar to ALU8_HUMAN A | 1 | 0.29 | T2-T4 grade 3 solid tumor marker |
| 415027 | D31010 | | gb: HUML 12147 Human fetal lung Homo sapie | 1 | 1 | T2-T4 grade 3 solid tumor marker |
| 416099 | H18626 | Hs.22634 | ESTs | 1 | 0.74 | T2-T4 grade 3 solid tumor marker |
| 416655 | AW968613 | Hs.79428 | BCL2/adenovirus E1B 19 kD-interacting pro | 3.8 | 0.12 | T2-T4 grade 3 solid tumor marker |
| 418329 | AW247430 | Hs.84152 | cystathionine-beta-synthase | 2.85 | 0.08 | T2-T4 grade 3 solid tumor marker |
| 420347 | AL033539 | Hs.97124 | Human DNA sequence from clone RP1-309H15 | 1 | 0.2 | T2-T4 grade 3 solid tumor marker |
| 421243 | AW873803 | Hs.102876 | pancreatic lipase | 1 | 0.38 | T2-T4 grade 3 solid tumor marker |
| 422660 | AW297582 | Hs.237062 | ESTs | 1.05 | 0.32 | T2-T4 grade 3 solid tumor marker |
| 422834 | AA318334 | | gb: EST20402 Retina II Homo sapiens cDNA | 1 | 0.38 | T2-T4 grade 3 solid tumor marker |
| 422972 | N59319 | Hs.145404 | ESTs | 1 | 0.61 | T2-T4 grade 3 solid tumor marker |
| 423104 | AJ005273 | Hs.123647 | antigenic determinant of recA protein (m | 2.95 | 0.12 | T2-T4 grade 3 solid tumor marker |
| 423634 | AW959908 | Hs.1690 | heparin-binding growth factor binding pr | 1 | 0.05 | T2-T4 grade 3 solid tumor marker |
| 424268 | AA397653 | Hs.144339 | Human DNA sequence from clone 495O10 on | 1 | 0.35 | T2-T4 grade 3 solid tumor marker |
| 425196 | AL037915 | Hs.155097 | carbonic anhydrase II | 2.75 | 0.05 | T2-T4 grade 3 solid tumor marker |
| 427099 | AB032953 | Hs.173560 | odd Oz/ten-m homolog 2 (Drosophila, mous | 1 | 0.09 | T2-T4 grade 3 solid tumor marker |
| 430300 | U60805 | Hs.238648 | oncostatin M receptor | 1 | 0.25 | T2-T4 grade 3 solid tumor marker |
| 431098 | AW501465 | Hs.249230 | ribonuclease L (2′,5′-oligoisoadenylate | 1 | 0.28 | T2-T4 grade 3 solid tumor marker |
| 431277 | AA501806 | Hs.249965 | ESTs | 1 | 0.22 | T2-T4 grade 3 solid tumor marker |
| 431750 | AA514986 | Hs.283705 | ESTs | 1 | 1 | T2-T4 grade 3 solid tumor marker |
| 434273 | AA913143 | Hs.26303 | ESTs | 1 | 0.41 | T2-T4 grade 3 solid tumor marker |
| 435505 | AF200492 | Hs.211238 | interleukin-1 homolog 1 | 1.8 | 0.19 | T2-T4 grade 3 solid tumor marker |
| 436281 | AW411194 | Hs.120051 | ESTs | 2.05 | 0.14 | T2-T4 grade 3 solid tumor marker |
| 437010 | AA741368 | Hs.291434 | ESTs | 2.4 | 0.17 | T2-T4 grade 3 solid tumor marker |
| 437814 | AI088192 | Hs.135474 | ESTs, Weakly similar to DDX9_HUMAN ATP-D | 1.8 | 0.13 | T2-T4 grade 3 solid tumor marker |
| 438361 | AA805666 | Hs.146217 | Homo sapiens cDNA: FLJ23077 fis, clone L | 1 | 0.3 | T2-T4 grade 3 solid tumor marker |
| 438376 | BE541211 | Hs.34804 | Homo sapiens cDNA FLJ11472 fis, clone HE | 1 | 0.57 | T2-T4 grade 3 solid tumor marker |
| 439370 | AW274369 | Hs.158853 | ESTs | 1 | 0.17 | T2-T4 grade 3 solid tumor marker |
| 440021 | AW025498 | Hs.270842 | ESTs, Weakly similar to ALU8_HUMAN ALU S | 1 | 0.65 | T2-T4 grade 3 solid tumor marker |
| 440404 | AI015881 | Hs.125616 | ESTs | 1 | 0.26 | T2-T4 grade 3 solid tumor marker |
| 441523 | AW514263 | Hs.168872 | ESTs, Weakly similar to ALUF_HUMAN !!!! | 1 | 1 | T2-T4 grade 3 solid tumor marker |
| 442277 | AW448914 | Hs.202391 | ESTs | 2.4 | 0.15 | T2-T4 grade 3 solid tumor marker |
| 442738 | AW002370 | Hs.1 31055 | ESTs | 1 | 0.29 | T2-T4 grade 3 solid tumor marker |
| 443297 | AI049864 | Hs.133029 | ESTs | 1 | 1 | T2-T4 grade 3 solid tumor marker |
| 444754 | T83911 | Hs.11881 | transmembrane 4 superfamily member 4 | 1 | 0.61 | T2-T4 grade 3 solid tumor marker |
| 445550 | AI242754 | Hs.137306 | ESTs | 1 | 0.5 | T2-T4 grade 3 solid tumor marker |
| 446149 | BE242960 | Hs.203181 | ESTs | 1 | 0.25 | T2-T4 grade 3 solid tumor marker |
| 446163 | AA026880 | Hs.25252 | Homo sapiens cDNA FLJ13603 fis, clone PL | 1 | 0.21 | T2-T4 grade 3 solid tumor marker |
| 446434 | AI823410 | Hs.169149 | karyopherin alpha 1 (importin alpha 5) | 1 | 1 | T2-T4 grade 3 solid tumor marker |
| 446928 | AI694493 | Hs.246916 | ESTs | 1 | 0.31 | T2-T4 grade 3 solid tumor marker |
| 448591 | AI540111 | Hs.171261 | ESTs | 1 | 1 | T2-T4 grade 3 solid tumor marker |
| 449121 | AI915858 | Hs.194980 | ESTs | 1 | 1 | T2-T4 grade 3 solid tumor marker |
| 449539 | W80363 | Hs.58446 | ESTs | 1 | 0.33 | T2-T4 grade 3 solid tumor marker |
| 450451 | AW591528 | Hs.202072 | ESTs | 1 | 0.59 | T2-T4 grade 3 solid tumor marker |
| 450469 | AI955049 | Hs.281326 | ESTs | 1 | 0.43 | T2-T4 grade 3 solid tumor marker |
| 450684 | AA872605 | Hs.25333 | interleukin 1 receptor, type II | 1 | 0.05 | T2-T4 grade 3 solid tumor marker |
| 451099 | R52795 | Hs.25954 | interleukin 13 receptor, alpha 2 | 1.55 | 0.11 | T2-T4 grade 3 solid tumor marker |
| 451106 | BE382701 | Hs.25960 | v-myc avian myelocytomatosis viral relat | 1 | 0.95 | T2-T4 grade 3 solid tumor marker |
| 451130 | AI762250 | Hs.211347 | ESTs | 3.65 | 0.15 | T2-T4 grade 3 solid tumor marker |
| 451412 | AW136378 | Hs.208060 | ESTs | 1 | 1 | T2-T4 grade 3 solid tumor marker |
| 451806 | NM_003729 | Hs.27076 | RNA 3′-terminal phosphate cyclase | 1.35 | 0.22 | T2-T4 grade 3 solid tumor marker |
| 452114 | N22687 | Hs.8236 | ESTs | 1 | 0.19 | T2-T4 grade 3 solid tumor marker |
| 452743 | AW965082 | Hs.61455 | ESTs | 1 | 0.44 | T2-T4 grade 3 solid tumor marker |
| 454622 | U70071 | | gb: HSU70071 Human Homo sapiens cDNA clon | 1 | 1 | T2-T4 grade 3 solid tumor marker |
| 455235 | AW875951 | | gb: CM1-PT0013-131299-067-f09 PT0013 Homo | 1 | 0.31 | T2-T4 grade 3 solid tumor marker |
| 457792 | AL046988 | Hs.268677 | ESTs, Moderately similar to ALU7_HUMAN A | 1 | 0.24 | T2-T4 grade 3 solid tumor marker |
| 100147 | D13666 | Hs.136348 | osteoblast specific factor 2 (fasciclin | 20.531 | 0.036 | upregulate stage |
| 101193 | L20861 | Hs.152213 | “wingless-type MMTV integration site fam | 1 | 0.526 | upregulate stage |
| 101724 | M69225 | Hs.620 | bullous pemphigoid antigen 1 (230/240 kD) | 20.67 | 0.037 | upregulate stage |
| 101809 | M86849 | “Hs.323733 | “gap junction protein, beta 2, 26 kD (con | 20.78 | 0.019 | upregulate stage |
| 102154 | U17760 | Hs.75517 | “laminin, beta 3 (nicein (125 kD), kalini | 18.848 | 0.042 | upregulate stage |
| 102211 | U23070 | Hs.78776 | putative transmembrane protein | 2.092 | 0.28 | upregulate stage |
| 102623 | U66083 | Hs.37110 | “melanoma antigen, family A, 9” | 1 | 0.306 | upregulate stage |
| 102915 | X07820 | Hs.2258 | matrix metalloproteinase 10 (stromelysin | 3.27 | 0.041 | upregulate stage |
| 103036 | X54925 | “Hs.83169 | matrix metalloproteinase 1 (interstitial | 13.63 | 0.034 | upregulate stage |
| 103119 | X63629 | Hs.2877 | “cadherin 3, type 1, P-cadherin (placent | 7.296 | 0.054 | upregulate stage |
| 103312 | X82693 | Hs.3185 | “lymphocyte antigen 6 complex, locus D” | 0.908 | 0.485 | upregulate stage |
| 103478 | Y07755 | Hs.38991 | S100 calcium-binding protein A2 | 2.928 | 0.219 | upregulate stage |
| 103587 | Z29083 | Hs.82128 | 5T4 oncofetal trophoblast glycoprotein | 3.156 | 0.16 | upregulate stage |
| 106632 | AA459897 | Hs.11950 | GPI-anchored metastasis-associated prote | 1.642 | 0.516 | upregulate stage |
| 107151 | AA621169 | Hs.8687 | ESTs | 2.421 | 0.174 | upregulate stage |
| 107901 | AA026418 | “Hs.111758 | keratin 6A | 1.259 | 0.343 | upregulate stage |
| 107922 | AA028028 | Hs.61460 | “Homo sapiens Ig superfamily receptor LN | 14.22 | 0.049 | upregulate stage |
| 109166 | AA179845 | Hs.73625 | “RAB6 interacting, kinesin like (rabkine | 11.13 | 0.039 | upregulate stage |
| 109424 | AA227919 | “Hs.85962 | hyaluronan synthase 3 | 1.737 | 0.518 | upregulate stage |
| 110906 | N39584 | Hs.17404 | ESTs | 20.93 | 0.021 | upregulate stage |
| 112244 | R51309 | Hs.70823 | KIAA1077 protein | 3.941 | 0.181 | upregulate stage |
| 115060 | AA253214 | Hs.198249 | “gap junction protein, beta 5 (connexin | 1.932 | 0.502 | upregulate stage |
| 115697 | AA411502 | Hs.63325 | “transmembrane protease, serine 4” | 7.394 | 0.101 | upregulate stage |
| 115978 | AA447522 | “Hs.69517 | “Homo sapiens, clone MGC: 5257, mRNA, com | 1.667 | 0.445 | upregulate stage |
| 116335 | AA495830 | Hs.41690 | desmocollin 3 | 4.899 | 0.154 | upregulate stage |
| 118314 | N63402 | Hs.46692 | ESTs | 9.75 | 0.069 | upregulate stage |
| 118336 | N63604 | Hs.47166 | HT021 | 4.601 | 0.197 | upregulate stage |
| 119845 | W79920 | Hs.58561 | G protein-coupled receptor 87 | 1.95 | 0.123 | upregulate stage |
| 120486 | AA253400 | Hs.137569 | tumor protein 63 kDa with strong homolog | 4.191 | 0.211 | upregulate stage |
| 121027 | AA398470 | Hs.99785 | “Homo sapiens cDNA: FLJ21245 fis, clone | 14.25 | 0.058 | upregulate stage |
| 124059 | F13673 | Hs.283713 | “ESTs, Weakly similar to ORF YGL050w [S. | 4.99 | 0.168 | upregulate stage |
| 128595 | U31875 | “Hs.152677 | “Homo sapiens cDNA FLJ20338 fis, clone H | 2.433 | 0.306 | upregulate stage |
| 128610 | L38608 | Hs.10247 | activated leucocyte cell adhesion molecu | 4.34 | 0.14 | upregulate stage |
| 129041 | H58873 | “Hs.169902 | “solute carrier family 2 (facilitated gI | 2.003 | 0.455 | upregulate stage |
| 129466 | L42583 | “Hs.111758 | keratin 6A | 11.584 | 0.042 | upregulate stage |
| 130627 | L23808 | Hs.1695 | matrix metalloproteinase 12 (macrophage | 2.376 | 0.233 | upregulate stage |
| 132349 | Y00705 | “Hs.181286 | “serine protease inhibitor, Kazal type 1 | 5.4 | 0.132 | upregulate stage |
| 132710 | W93726 | Hs.55279 | “seine (or cysteine) proteinase inhibit | 3.888 | 0.187 | upregulate stage |
| 133391 | X57579 | Hs.727 | “inhibin, beta A (activin A, activin AB | 1.517 | 0.334 | upregulate stage |
| 134110 | AA242758 | “Hs.79136 | “LIV-1 protein, estrogen regulated” | 2.221 | 0.387 | upregulate stage |
| 400289 | X07820 | Hs.2258 | matrix metalloproteinase 10 (stromelysin | 4.85 | 0.03 | upregulate stage |
| 400297 | AI127076 | Hs.288381 | hypothetical protein DKFZp564O1278 | 3.54 | 0.13 | upregulate stage |
| 400346 | AB041269 | Hs.272263 | Homo sapiens mRNA for keratin 19, partia | 8.95 | 0.07 | upregulate stage |
| 400419 | AF084545 | Hs.81800 | chondroitin sulfate proteoglycan 2 (vers | 10.7 | 0.06 | upregulate stage |
| 400495 | | | | 1 | 0.56 | upregulate stage |
| 400509 | M97639 | Hs.155585 | receptor tyrosine kinase-like orphan rec | 1.52 | 0.51 | upregulate stage |
| 400528 | | | | 3.47 | 0.23 | upregulate stage |
| 400577 | | | | 1 | 0.29 | upregulate stage |
| 400608 | | | | 7.2 | 0.08 | upregulate stage |
| 400644 | | | | 1 | 1 | upregulate stage |
| 400666 | | | | 1.42 | 0.43 | upregulate stage |
| 400750 | | | | 8.7 | 0.1 | upregulate stage |
| 400773 | | | | 1.11 | 0.51 | upregulate stage |
| 400844 | | | | 9.65 | 0.04 | upregulate stage |
| 400845 | | | | 2.3 | 0.28 | upregulate stage |
| 400846 | | | | 1.34 | 0.5 | upregulate stage |
| 400880 | | | | 9.4 | 0.06 | upregulate stage |
| 400887 | | | | 1 | 1 | upregulate stage |
| 401086 | | | | 1 | 0.51 | upregulate stage |
| 401093 | | | | 7 | 0.08 | upregulate stage |
| 401101 | | | | 1 | 0.17 | upregulate stage |
| 401197 | | | | 5.18 | 0.14 | upregulate stage |
| 401262 | | | | 1 | 1 | upregulate stage |
| 401271 | | | | 1 | 1 | upregulate stage |
| 401279 | | | | 9.1 | 0.06 | upregulate stage |
| 401342 | | | | 1.42 | 0.5 | upregulate stage |
| 401345 | M83738 | Hs.147663 | protein tyrosine phosphatase, non-recept | 1 | 0.33 | upregulate stage |
| 401365 | | | | 6.5 | 0.11 | upregulate stage |
| 401395 | | | | 1 | 0.31 | upregulate stage |
| 401420 | | | | 1 | 1 | upregulate stage |
| 401439 | | | | 2.65 | 0.17 | upregulate stage |
| 401451 | | | | 12 | 0.05 | upregulate stage |
| 401599 | BE247275 | Hs.151787 | U5 snRNP-specific protein, 116 kD | 9.15 | 0.08 | upregulate stage |
| 401600 | BE247275 | Hs.151787 | U5 snRNP-specific protein, 116 kD | 8.75 | 0.09 | upregulate stage |
| 401694 | | | | 1 | 1 | upregulate stage |
| 401747 | | | | 29.75 | 0.02 | upregulate stage |
| 401759 | | | | 11.35 | 0.06 | upregulate stage |
| 401780 | | | | 6.15 | 0.07 | upregulate stage |
| 401868 | AC005261 | Hs.98338 | serine/threonine kinase 13 (aurora/IPL1- | 1 | 0.69 | upregulate stage |
| 401994 | | | | 3.15 | 0.15 | upregulate stage |
| 402001 | | | | 4.4 | 0.14 | upregulate stage |
| 402230 | | | | 8.75 | 0.06 | upregulate stage |
| 402325 | | | | 1 | 0.36 | upregulate stage |
| 402408 | | | | 5.15 | 0.1 | upregulate stage |
| 402472 | | | | 9.05 | 0.08 | upregulate stage |
| 402480 | | | | 1 | 1 | upregulate stage |
| 402490 | | | | 9.6 | 0.07 | upregulate stage |
| 402553 | | | | 9.85 | 0.09 | upregulate stage |
| 402889 | | | | 9.4 | 0.09 | upregulate stage |
| 402901 | | | | 1.07 | 0.65 | upregulate stage |
| 402938 | | | | 1 | 1 | upregulate stage |
| 402995 | | | | 9.6 | 0.06 | upregulate stage |
| 403005 | | | | 1.5 | 0.21 | upregulate stage |
| 403020 | | | | 5.15 | 0.12 | upregulate stage |
| 403052 | R58624 | Hs.2186 | eukaryotic translation elongation factor | 1 | 1 | upregulate stage |
| 403053 | R58624 | Hs.2186 | eukaryotic translation elongation factor | 1.5 | 0.28 | upregulate stage |
| 403073 | | | | 1 | 0.37 | upregulate stage |
| 403085 | | | | 1 | 0.43 | upregulate stage |
| 403106 | | | | 1.12 | 0.57 | upregulate stage |
| 403152 | AA576664 | Hs.37078 | v-crk avian sarcoma virus CT10 oncogene | 0.86 | 1.08 | upregulate stage |
| 403172 | | | | 7.7 | 0.09 | upregulate stage |
| 403212 | | | | 1.18 | 0.62 | upregulate stage |
| 403214 | | | | 6.05 | 0.1 | upregulate stage |
| 403277 | | | | 4.5 | 0.11 | upregulate stage |
| 403331 | | | | 3.2 | 0.13 | upregulate stage |
| 403381 | | | | 10.7 | 0.05 | upregulate stage |
| 403485 | | | | 10.35 | 0.08 | upregulate stage |
| 403588 | | | | 1 | 1 | upregulate stage |
| 403851 | | | | 2.45 | 0.34 | upregulate stage |
| 403860 | | | | 1 | 1 | upregulate stage |
| 403894 | | | | 4.45 | 0.14 | upregulate stage |
| 403903 | | | | 1.39 | 0.58 | upregulate stage |
| 403954 | W28077 | Hs.79389 | nel (chicken)-like 2 | 1 | 1 | upregulate stage |
| 404148 | | | | 9.15 | 0.08 | upregulate stage |
| 404229 | | | | 1 | 1 | upregulate stage |
| 404268 | | | | 1 | 1 | upregulate stage |
| 404274 | | | | 1.3 | 0.2 | upregulate stage |
| 404288 | | | | 1 | 0.39 | upregulate stage |
| 404403 | | | | 1 | 0.28 | upregulate stage |
| 404440 | | | | 7.05 | 0.06 | upregulate stage |
| 404507 | | | | 1 | 0.33 | upregulate stage |
| 404516 | | | | 1 | 1 | upregulate stage |
| 404639 | | | | 1 | 1 | upregulate stage |
| 404684 | | | | 0.89 | 0.9 | upregulate stage |
| 404685 | | | | 2.74 | 0.26 | upregulate stage |
| 404704 | | | | 9.35 | 0.08 | upregulate stage |
| 404829 | | | | 1 | 0.24 | upregulate stage |
| 404860 | | | | 3.65 | 0.15 | upregulate stage |
| 404894 | | | | 2.05 | 0.16 | upregulate stage |
| 404939 | | | | 1 | 1 | upregulate stage |
| 405034 | AL035754 | Hs.2474 | toll-like receptor 1 | 1 | 0.18 | upregulate stage |
| 405059 | | | | 1 | 0.56 | upregulate stage |
| 405064 | | | | 1 | 0.22 | upregulate stage |
| 405102 | | | | 9.65 | 0.08 | upregulate stage |
| 405167 | | | | 1 | 0.67 | upregulate stage |
| 405170 | | | | 1 | 0.48 | upregulate stage |
| 405177 | | | | 1 | 0.22 | upregulate stage |
| 405186 | | | | 3.75 | 0.1 | upregulate stage |
| 405258 | | | | 8.85 | 0.09 | upregulate stage |
| 405281 | | | | 1 | 1 | upregulate stage |
| 405379 | | | | 1 | 0.87 | upregulate stage |
| 405494 | | | | 5 | 0.13 | upregulate stage |
| 405520 | | | | 1 | 0.95 | upregulate stage |
| 405526 | | | | 8.96 | 0.08 | upregulate stage |
| 405725 | | | | 3.3 | 0.12 | upregulate stage |
| 405738 | | | | 0.86 | 0.69 | upregulate stage |
| 405809 | | | | 2.4 | 0.18 | upregulate stage |
| 405838 | | | | 1 | 0.22 | upregulate stage |
| 405906 | | | | 2.6 | 0.12 | upregulate stage |
| 406137 | | | | 1.54 | 0.52 | upregulate stage |
| 406187 | | | | 3.2 | 0.14 | upregulate stage |
| 406322 | | | | 3.95 | 0.12 | upregulate stage |
| 406360 | | | | 4.1 | 0.1 | upregulate stage |
| 406397 | | | | 1 | 0.24 | upregulate stage |
| 406434 | | | | 7.4 | 0.07 | upregulate stage |
| 406467 | | | | 9.1 | 0.07 | upregulate stage |
| 406511 | | | | 1 | 1 | upregulate stage |
| 406517 | W28077 | Hs.79389 | nel (chicken)-like 2 | 1 | 1 | upregulate stage |
| 406588 | | | | 0.93 | 0.91 | upregulate stage |
| 406651 | AI559224 | Hs.277477 | major histocompatibility complex, class | 10.1 | 0.07 | upregulate stage |
| 406665 | U22961 | Hs.75442 | albumin | 1.08 | 0.81 | upregulate stage |
| 406671 | AA129547 | Hs.285754 | met proto-oncogene (hepatocyte growth fa | 5.7 | 0.12 | upregulate stage |
| 406687 | M31126 | Hs.272620 | pregnancy specific beta-1-glycoprotein 9 | 1.95 | 0.3 | upregulate stage |
| 406732 | AA487229 | Hs.2064 | vimentin | 1 | 0.77 | upregulate stage |
| 406747 | AI925153 | Hs.217493 | annexin A2 | 3.6 | 0.14 | upregulate stage |
| 406753 | AA505665 | Hs.217493 | annexin A2 | 5.45 | 0.13 | upregulate stage |
| 406815 | AA833930 | Hs.288036 | tRNA isopentenylpyrophosphate transferas | 3.65 | 0.09 | upregulate stage |
| 406850 | AI624300 | Hs.172928 | collagen, type I, alpha 1 | 1.29 | 0.62 | upregulate stage |
| 406892 | D55643 | | gb: Human spleen PABL (pseudoautosomal bo | 1 | 1 | upregulate stage |
| 406944 | J04742 | Hs.247945 | Human autonomous replicating sequence H1 | 1 | 1 | upregulate stage |
| 406950 | L17325 | Hs.278 | pre-T/NK cell associated protein | 1 | 0.36 | upregulate stage |
| 406961 | L77563 | | gb: Homo sapiens DGS-F partial mRNA. | 1 | 1 | upregulate stage |
| 406964 | M21305 | Hs.247946 | Human alpha satellite and satellite 3 ju | 42.25 | 0.01 | upregulate stage |
| 406993 | S83249 | | gb: NG-TRA = transporter protein/putative h | 1 | 1 | upregulate stage |
| 407017 | U48697 | | gb: Human mariner-like element-containing | 1 | 1 | upregulate stage |
| 407073 | Y10510 | | gb: H. sapiens mRNA for CD67S protein. | 1 | 0.53 | upregulate stage |
| 407105 | S64699 | Hs.663 | cystic fibrosis transmembrane conductanc | 1 | 1 | upregulate stage |
| 407128 | R83312 | Hs.237260 | EST | 1 | 1 | upregulate stage |
| 407132 | T02871 | Hs.228523 | EST | 1 | 0.45 | upregulate stage |
| 407137 | T97307 | Hs.199067 | v-erb-b2 avian erythroblastic leukemia v | 14.3 | 0.05 | upregulate stage |
| 407158 | N49839 | | gb: yz08b10.s1 Soares multiple_sclerosis_{—} | 1 | 0.57 | upregulate stage |
| 407175 | T86603 | | gb: yd87d12.s1 Soares fetal liver spleen | 1 | 0.31 | upregulate stage |
| 407186 | AA435610 | | gb: zt74b11.s1 Soares_testis_NHT Homo sap | 1 | 1 | upregulate stage |
| 407189 | AA598927 | | gb: ae37e03.s1 Gessler Wilms tumor Homo s | 1 | 1 | upregulate stage |
| 407192 | AA609200 | | gb: af12e02.s1 Soares_testis_NHT Homo sap | 6.05 | 0.12 | upregulate stage |
| 407195 | C21124 | | gb: HUMGS0002072 Human adult (K. Okubo) Ho | 1 | 1 | upregulate stage |
| 407202 | N58172 | Hs.109370 | ESTs | 3.7 | 0.16 | upregulate stage |
| 407204 | R41933 | Hs.140237 | ESTs, Weakly similar to AF119917 13 PRO1 | 10.2 | 0.06 | upregulate stage |
| 407205 | R78910 | Hs.272620 | pregnancy specific beta-1-glycoprotein 9 | 1.9 | 0.22 | upregulate stage |
| 407211 | T95828 | Hs.230070 | EST | 1 | 0.59 | upregulate stage |
| 407346 | AI090210 | Hs.264106 | ESTs | 1 | 1 | upregulate stage |
| 407422 | AF116633 | | gb: Homo sapiens PRO1318 mRNA, complete c | 1 | 0.22 | upregulate stage |
| 407494 | U10072 | | gb: Human forkhead family (AFX1) mRNA, pa | 4.1 | 0.13 | upregulate stage |
| 407547 | Y10259 | | gb: H. sapiens ACTH receptor mRNA 3′UTR. | 2.45 | 0.19 | upregulate stage |
| 407564 | AA042860 | Hs.103005 | ESTs | 1 | 1 | upregulate stage |
| 407603 | AW955705 | Hs.62604 | ESTs | 1.18 | 0.73 | upregulate stage |
| 407634 | AW016569 | Hs.301280 | ESTs, Highly similar to AF241831 1 intra | 9.6 | 0.06 | upregulate stage |
| 407668 | BE161086 | Hs.279817 | ESTs | 1 | 0.39 | upregulate stage |
| 407709 | AA456135 | Hs.23023 | ESTs | 6.8 | 0.12 | upregulate stage |
| 407710 | AW022727 | Hs.23616 | ESTs | 3.9 | 0.14 | upregulate stage |
| 407725 | BE388094 | Hs.21857 | ESTs | 9.97 | 0.07 | upregulate stage |
| 407729 | T40707 | Hs.270862 | ESTs | 9.2 | 0.09 | upregulate stage |
| 407774 | AA084958 | | gb: zn13d12.r1 Stratagene hNT neuron (937 | 2.65 | 0.22 | upregulate stage |
| 407788 | BE514982 | Hs.38991 | S100 calcium-binding protein A2 | 2.1 | 0.34 | upregulate stage |
| 407811 | AW190902 | Hs.40098 | cysteine knot superfamily 1, BMP antagon | 8.45 | 0.06 | upregulate stage |
| 407813 | AL120247 | Hs.40109 | KIAA0872 protein | 9.1 | 0.08 | upregulate stage |
| 407833 | AW955632 | Hs.66666 | ESTs | 9.2 | 0.07 | upregulate stage |
| 407839 | AA045144 | Hs.161566 | ESTs | 2.11 | 0.25 | upregulate stage |
| 407853 | AA336797 | Hs.40499 | dickkopf (Xenopus laevis) homolog 1 | 1 | 0.34 | upregulate stage |
| 407881 | AW072003 | Hs.40968 | heparan sulfate (glucosamine) 3-O-sulfot | 3.52 | 0.18 | upregulate stage |
| 407882 | AI241264 | Hs.62772 | ESTs | 1 | 0.26 | upregulate stage |
| 407910 | AA650274 | Hs.41296 | fibronectin leucine rich transmembrane p | 13.6 | 0.05 | upregulate stage |
| 407911 | AF104922 | Hs.41565 | growth differentiation factor 8 | 1 | 1 | upregulate stage |
| 407912 | AW104401 | Hs.243489 | ESTs, Weakly similar to AF151881 1 CGI-1 | 10.35 | 0.07 | upregulate stage |
| 407935 | U31986 | Hs.41683 | cartilage paired-class homeoprotein 1 | 4.25 | 0.12 | upregulate stage |
| 407939 | W05608 | | gb: za85e07.r1 Soares_fetal_lung_NbHL19W | 8.75 | 0.09 | upregulate stage |
| 407944 | R34008 | Hs.239727 | desmocollin 2 | 9.2 | 0.06 | upregulate stage |
| 407945 | X69208 | Hs.606 | ATPase, Cu++ transporting, alpha polypep | 1.45 | 0.25 | upregulate stage |
| 407946 | AA226495 | Hs.154292 | ESTs | 9.4 | 0.07 | upregulate stage |
| 407949 | W21874 | Hs.247057 | ESTs | 3.32 | 0.2 | upregulate stage |
| 407974 | AW968123 | Hs.146401 | small inducible cytokine subfamily E, me | 3.55 | 0.14 | upregulate stage |
| 407983 | U40371 | Hs.41718 | phosphodiesterase 1C, calmodulin-depende | 8.95 | 0.07 | upregulate stage |
| 407994 | AW135309 | Hs.244331 | ESTs | 4.5 | 0.12 | upregulate stage |
| 408000 | L11690 | Hs.620 | bullous pemphigoid antigen 1 (230/240 kD) | 2.89 | 0.19 | upregulate stage |
| 408014 | AA723782 | Hs.41749 | protein kinase, cGMP-dependent, type II | 1.31 | 0.53 | upregulate stage |
| 408031 | AA081395 | Hs.42173 | Homo sapiens cDNA FLJ10366 fis, clone NT | 3.6 | 0.17 | upregulate stage |
| 408046 | AW139121 | Hs.183643 | ESTs | 1 | 0.36 | upregulate stage |
| 408063 | BE086548 | Hs.42346 | calcineurin-binding protein calsarcin-1 | 10.75 | 0.05 | upregulate stage |
| 408092 | NM_007057 | Hs.42650 | ZW10 interactor | 4.7 | 0.13 | upregulate stage |
| 408101 | AW968504 | Hs.123073 | CDC2-related protein kinase 7 | 4.5 | 0.14 | upregulate stage |
| 408141 | U69205 | Hs.45152 | ESTs, Moderately similar to neurogenic b | 4.4 | 0.13 | upregulate stage |
| 408170 | AW204516 | Hs.31835 | ESTs | 5.85 | 0.13 | upregulate stage |
| 408184 | AW168741 | Hs.22249 | ESTs | 1 | 1 | upregulate stage |
| 408224 | AW175997 | | gb: QV0-BT0078-190899-005-E02 BT0078 Homo | 1 | 0.44 | upregulate stage |
| 408239 | AA053401 | Hs.271827 | ESTs, Moderately similar to ALU7_HUMAN A | 9.95 | 0.04 | upregulate stage |
| 408241 | AW176546 | | gb: MR0-CT0063-200899-001-a01 CT0063 Homo | 2.8 | 0.17 | upregulate stage |
| 408268 | AL138247 | | gb: DKFZp547D237_r1 547 (synonym: hfbr1) | 1 | 0.61 | upregulate stage |
| 408277 | AW177959 | | gb: IL3-HT0060-200899-008-D03 HT0060 Homo | 1 | 1 | upregulate stage |
| 408306 | BE141991 | | gb: PM2-HT0134-220999-002-d10 HT0134 Homo | 1 | 1 | upregulate stage |
| 408352 | AA053875 | Hs.95310 | ESTs | 1 | 1 | upregulate stage |
| 408360 | AI806090 | Hs.44344 | hypothetical protein FLJ20534 | 9.15 | 0.08 | upregulate stage |
| 408393 | AW015318 | Hs.23165 | ESTs | 9.35 | 0.07 | upregulate stage |
| 408396 | AA330496 | Hs.40840 | ESTs | 1 | 0.61 | upregulate stage |
| 408442 | R59608 | Hs.21435 | ESTs | 1 | 1 | upregulate stage |
| 408514 | AW206559 | Hs.255903 | ESTs | 1 | 0.34 | upregulate stage |
| 408572 | AA055611 | Hs.226568 | ESTs, Moderately similar to ALU4_HUMAN A | 1 | 0.33 | upregulate stage |
| 408617 | R61736 | Hs.124128 | ESTs | 2.75 | 0.14 | upregulate stage |
| 408633 | AW963372 | Hs.46677 | PRO2000 protein | 3.14 | 0.25 | upregulate stage |
| 408706 | AW438503 | Hs.256935 | ESTs | 8.45 | 0.09 | upregulate stage |
| 408713 | NM_001248 | Hs.47042 | ectonucleoside triphosphate diphosphohyd | 2.81 | 0.21 | upregulate stage |
| 408725 | AA131539 | Hs.15669 | ESTs | 9.1 | 0.08 | upregulate stage |
| 408728 | AL137379 | Hs.47125 | hypothetical protein FLJ13912 | 3.1 | 0.11 | upregulate stage |
| 408738 | NM_014785 | Hs.47313 | KIAA0258 gene product | 4.4 | 0.13 | upregulate stage |
| 408739 | W01556 | Hs.238797 | ESTs | 5.65 | 0.11 | upregulate stage |
| 408754 | N31256 | Hs.161623 | ESTs | 1 | 1 | upregulate stage |
| 408765 | AA057268 | Hs.146013 | ESTs | 8.75 | 0.09 | upregulate stage |
| 408805 | H69912 | Hs.48269 | vaccinia related kinase 1 | 4.95 | 0.12 | upregulate stage |
| 408813 | AI580090 | Hs.48295 | RNA helicase family | 3.65 | 0.17 | upregulate stage |
| 408817 | AA524525 | Hs.279864 | PRO1996 protein | 6.15 | 0.12 | upregulate stage |
| 408849 | BE219451 | Hs.254919 | ESTs | 1 | 0.32 | upregulate stage |
| 408902 | AW014869 | Hs.5510 | ESTs | 3.3 | 0.15 | upregulate stage |
| 408908 | BE296227 | Hs.48915 | serine/threonine kinase 15 | 5.65 | 0.1 | upregulate stage |
| 408916 | AW295232 | Hs.22893 | ESTs | 10 | 0.08 | upregulate stage |
| 408933 | AA058979 | Hs.182133 | ESTs, Highly similar to ADP-ribosylation | 1 | 0.91 | upregulate stage |
| 408943 | NM_007070 | Hs.49105 | FKBP-associated protein | 3.45 | 0.16 | upregulate stage |
| 408960 | BE158389 | Hs.300976 | ESTs | 6.3 | 0.1 | upregulate stage |
| 409032 | AW301807 | Hs.297260 | ESTs | 8.4 | 0.08 | upregulate stage |
| 409093 | BE243834 | Hs.50441 | CGI-04 protein | 1.71 | 0.49 | upregulate stage |
| 409099 | AK000725 | Hs.50579 | hypothetical protein FLJ20718 | 10.1 | 0.07 | upregulate stage |
| 409142 | AL136877 | Hs.50758 | chromosome-associated polypeptide C | 11.85 | 0.05 | upregulate stage |
| 409203 | AA780473 | Hs.687 | cytochrome P450, subfamily IVB, polypept | 2.83 | 0.24 | upregulate stage |
| 409231 | AA446644 | Hs.692 | tumor-associated calcium signal transduc | 9.34 | 0.08 | upregulate stage |
| 409262 | AK000631 | Hs.52256 | hypothetical protein FLJ20624 | 8.7 | 0.09 | upregulate stage |
| 409357 | M73628 | Hs.54415 | casein, kappa | 1.6 | 0.2 | upregulate stage |
| 409402 | AF208234 | Hs.695 | cystatin B (stefin B) | 1.57 | 0.56 | upregulate stage |
| 409405 | AA075869 | Hs.126400 | ESTs, Highly similar to RL39_HUMAN 60S R | 2.6 | 0.12 | upregulate stage |
| 409408 | AW387837 | | gb: MR4-ST0118-021299-021-f08 ST0118 Homo | 4.3 | 0.15 | upregulate stage |
| 409420 | Z15008 | Hs.54451 | laminin, gamma 2 (nicein (100 kD), kalini | 8.28 | 0.06 | upregulate stage |
| 409509 | AL036923 | Hs.127006 | ESTs | 10.2 | 0.06 | upregulate stage |
| 409566 | AA078899 | | gb: zm94b01.r1 Stratagene colon HT29 (937 | 1 | 0.56 | upregulate stage |
| 409575 | AW419225 | Hs.256247 | ESTs | 2.15 | 0.14 | upregulate stage |
| 409582 | R27430 | Hs.271565 | ESTs | 7.3 | 0.07 | upregulate stage |
| 409632 | W74001 | Hs.55279 | serine (or cysteine) proteinase inhibito | 3.78 | 0.19 | upregulate stage |
| 409642 | AW450809 | Hs.257347 | ESTs | 9.55 | 0.07 | upregulate stage |
| 409674 | AI935146 | Hs.278611 | UDP-N-acetyl-alpha-D-galactosamine: polyp | 1 | 0.29 | upregulate stage |
| 409691 | T89983 | Hs.246042 | ESTs | 1 | 1 | upregulate stage |
| 409703 | NM_006187 | Hs.56009 | 2′-5′ oligoadenylate synthetase 3 | 2.22 | 0.36 | upregulate stage |
| 409727 | N63786 | Hs.94149 | ESTs, Weakly similar to ALU1_HUMAN ALU S | 1 | 0.57 | upregulate stage |
| 409760 | AA302840 | | gb: EST10534 Adipose tissue, white I Homo | 9.95 | 0.06 | upregulate stage |
| 409789 | BE256027 | Hs.180946 | ribosomal protein L5 | 1 | 0.83 | upregulate stage |
| 409794 | AW885691 | | gb: RC4-OT0071-240300-013-b04 OT0071 Homo | 1 | 1 | upregulate stage |
| 409977 | AW805510 | Hs.97056 | hypothetical protein FLJ21634 | 9.65 | 0.07 | upregulate stage |
| 409985 | AW291944 | Hs.122139 | ESTs | 4.35 | 0.14 | upregulate stage |
| 409989 | R37868 | Hs.13333 | ESTs | 1 | 0.21 | upregulate stage |
| 409995 | AW960597 | Hs.30164 | ESTs | 5.05 | 0.12 | upregulate stage |
| 410013 | AF067173 | Hs.57904 | mago-nashi (Drosophila) homolog, prolife | 3.05 | 0.26 | upregulate stage |
| 410044 | BE566742 | Hs.58169 | highly expressed in cancer, rich in leuc | 3.15 | 0.09 | upregulate stage |
| 410071 | AW582568 | | gb: RC1-ST0278-080100-011-h04 ST0278 Home | 2.5 | 0.18 | upregulate stage |
| 410102 | AW248508 | Hs.279727 | Homo sapiens cDNA FLJ14035 fis, clone HE | 8 | 0.06 | upregulate stage |
| 410114 | AW590540 | Hs.271280 | ESTs | 5.1 | 0.14 | upregulate stage |
| 410117 | AK001586 | Hs.58650 | hypothetical protein FLJ10724 | 1 | 1 | upregulate stage |
| 410153 | BE311926 | Hs.15830 | Homo sapiens cDNA FLJ12691 fis, clone NT | 4.7 | 0.11 | upregulate stage |
| 410181 | AI468210 | Hs.261285 | pleiotropic regulator 1 (PRL1, Arabidops | 1 | 0.23 | upregulate stage |
| 410196 | AI936442 | Hs.59838 | hypothetical protein FLJ10808 | 6.05 | 0.09 | upregulate stage |
| 410252 | AW821182 | Hs.61418 | microfibrillar-associated protein 1 | 5.55 | 0.12 | upregulate stage |
| 410259 | AK000337 | Hs.61485 | hypothetical protein | 10.1 | 0.07 | upregulate stage |
| 410276 | AI554545 | Hs.68301 | ESTs | 2.98 | 0.25 | upregulate stage |
| 410278 | AW614396 | Hs.282230 | ESTs | 1 | 0.28 | upregulate stage |
| 410325 | AB023154 | Hs.62264 | KIAA0937 protein | 6.85 | 0.13 | upregulate stage |
| 410356 | BE244668 | Hs.62643 | dual adaptor of phosphotyrosine and 3-ph | 1 | 1 | upregulate stage |
| 410388 | AA831460 | Hs.22039 | hepatocyte nuclear factor 3, alpha | 1 | 0.33 | upregulate stage |
| 410399 | BE068889 | Hs.63236 | synuclein, gamma (breast cancer-specific | 1.07 | 0.78 | upregulate stage |
| 410420 | AA224053 | Hs.172405 | ESTs, Moderately similar to I52835 H-NUC | 1 | 0.14 | upregulate stage |
| 410429 | AA310600 | Hs.63657 | hypothetical protein FLJ11005 | 11.25 | 0.07 | upregulate stage |
| 410442 | X73424 | Hs.63788 | propionyl Coenzyme A carboxylase, beta p | 9.6 | 0.08 | upregulate stage |
| 410475 | AW749927 | | gb: QV0-BT0537-231299-049-f03 BT0537 Home | 9.8 | 0.08 | upregulate stage |
| 410495 | N95428 | | gb: zb80d09.s1 Soares_senescent_fibroblas | 11.3 | 0.06 | upregulate stage |
| 410501 | AI675688 | Hs.83286 | ESTs | 4.75 | 0.1 | upregulate stage |
| 410503 | AW975746 | Hs.188662 | Homo sapiens cDNA: FLJ23421 fis, clone H | 6.5 | 0.1 | upregulate stage |
| 410520 | AW752710 | | gb: IL3-CT0219-281099-024-A03 CT0219 Homo | 1 | 1 | upregulate stage |
| 410534 | AW905138 | | gb: QV0-NN1071-280400-207-g07 NN1071 Homo | 3.1 | 0.16 | upregulate stage |
| 410537 | AW753108 | | gb: PM1-CT0247-080100-008-e10 CT0247 Homo | 10.35 | 0.08 | upregulate stage |
| 410553 | AW016824 | Hs.68784 | ESTs | 1.67 | 0.41 | upregulate stage |
| 410560 | N29220 | | gb: yx43b05.r1 Soares melanocyte 2NbHM Ho | 9 | 0.07 | upregulate stage |
| 410561 | BE540255 | Hs.6994 | Homo sapiens cDNA: FLJ22044 fis, clone H | 6.2 | 0.11 | upregulate stage |
| 410562 | AW858528 | | gb: CM3-CT0341-150300-119-h11 CT0341 Homo | 1 | 1 | upregulate stage |
| 410579 | AK001628 | Hs.64691 | KIAA0483 protein | 11.1 | 0.06 | upregulate stage |
| 410634 | AW888653 | Hs.266859 | ESTs | 1 | 1 | upregulate stage |
| 410664 | NM_006033 | Hs.65370 | lipase, endothelial | 3.95 | 0.1 | upregulate stage |
| 410668 | BE379794 | Hs.65403 | hypothetical protein | 1.82 | 0.41 | upregulate stage |
| 410730 | AW368860 | Hs.293950 | ESTs | 9.25 | 0.07 | upregulate stage |
| 410751 | AA357918 | | gb: EST66726 Fetal lung III Homo sapiens | 1 | 1 | upregulate stage |
| 410754 | T63840 | | gb: yc16b10.s1 Stratagene lung (937210) H | 3.1 | 0.14 | upregulate stage |
| 410762 | AF226053 | Hs.66170 | HSKM-B protein | 5.55 | 0.1 | upregulate stage |
| 410764 | AW978159 | Hs.250164 | ESTs, Weakly similar to coded for by C. | 1 | 0.2 | upregulate stage |
| 410782 | AW504860 | Hs.288836 | Homo sapiens cDNA FLJ12673 fis, clone NT | 1.75 | 0.25 | upregulate stage |
| 410794 | AA248010 | Hs.154669 | ESTs | 1 | 0.67 | upregulate stage |
| 410804 | U64820 | Hs.66521 | Machado-Joseph disease (spinocerebellar | 3.2 | 0.17 | upregulate stage |
| 410844 | AW807073 | | gb: MR4-ST0062-031199-018-d06 ST0062 Homo | 1 | 0.8 | upregulate stage |
| 410855 | X97795 | Hs.66718 | RAD54 (S. cerivisiae)-like | 6.5 | 0.12 | upregulate stage |
| 410910 | AW810204 | | gb: MR4-ST0125-021199-017-d08 ST0125 Homo | 9.35 | 0.08 | upregulate stage |
| 410973 | AW812278 | | gb: RC0-ST0174-211099-011-h12 ST0174 Homo | 1 | 1 | upregulate stage |
| 410976 | R36207 | Hs.25092 | ESTs | 8.35 | 0.1 | upregulate stage |
| 410997 | AW812877 | | gb: RC3-ST0186-300100-017-e04 ST0186 Homo | 1 | 1 | upregulate stage |
| 410998 | W28247 | Hs.82007 | KIAA0094 protein | 2.45 | 0.18 | upregulate stage |
| 411036 | AA857218 | Hs.297007 | ESTs | 4.05 | 0.14 | upregulate stage |
| 411110 | H93000 | | gb: yv07f01.s1 Soares fetal liver spleen | 1 | 0.36 | upregulate stage |
| 411132 | AW819191 | | gb: CM1-ST0283-071299-061-d08 ST0283 Homo | 1 | 1 | upregulate stage |
| 411137 | AW819455 | | gb: RC5-ST0293-021299-031-A04 ST0293 Homo | 3.65 | 0.18 | upregulate stage |
| 411157 | AW819867 | | gb: QV0-ST0294-070300-151-f02 ST0294 Homo | 3.2 | 0.2 | upregulate stage |
| 411159 | AW820178 | | gb: QV0-ST0294-100400-185-e07 ST0294 Homo | 1 | 0.27 | upregulate stage |
| 411170 | AW820503 | | gb: QV2-ST0298-140200-042-b05 ST0298 Homo | 1 | 1 | upregulate stage |
| 411193 | AW821484 | | gb: IL2-ST0311-211299-028-F12 ST0311 Homo | 1 | 0.24 | upregulate stage |
| 411242 | BE146808 | | gb: QV4-HT0222-181099-013-g03 HT0222 Homo | 2.55 | 0.26 | upregulate stage |
| 411245 | AW833441 | | gb: QV4-TT0008-271099-020-g01 TT0008 Homo | 9.62 | 0.09 | upregulate stage |
| 411263 | BE297802 | Hs.69360 | kinesin-like 6 (mitotic centromere-assoc | 2.4 | 0.32 | upregulate stage |
| 411282 | AW995011 | | gb: QV0-BN0040-170300-161-d07 BN0040 Homo | 1 | 1 | upregulate stage |
| 411284 | N28519 | Hs.135191 | ESTs, Weakly similar to unnamed protein | 3.25 | 0.12 | upregulate stage |
| 411294 | AW859729 | Hs.42680 | ESTs | 1 | 1 | upregulate stage |
| 411327 | AW836922 | | gb: QV1-LT0036-150200-074-h06 LT0036 Homo | 1 | 0.37 | upregulate stage |
| 411338 | AW731782 | Hs.116122 | ESTs, Weakly similar to unnamed protein | 5 | 0.13 | upregulate stage |
| 411339 | BE164598 | | gb: RC3-HT0470-120200-013-b10 HT0470 Homo | 1 | 0.25 | upregulate stage |
| 411383 | AA001394 | Hs.69749 | KIAA0087 gene product | 3.6 | 0.18 | upregulate stage |
| 411387 | AW842339 | Hs.130815 | hypothetical protein FLJ21870 | 8.75 | 0.09 | upregulate stage |
| 411400 | AA311919 | Hs.69851 | GAR1 protein | 12.1 | 0.07 | upregulate stage |
| 411425 | AW846012 | | gb: RC2-CT0163-230999-003-E01 CT0163 Homo | 1 | 0.74 | upregulate stage |
| 411461 | AW847937 | | gb: IL3-CT0213-210200-042-D02 CT0213 Homo | 1 | 1 | upregulate stage |
| 411526 | AW850327 | | gb: IL3-CT0219-221199-029-D08 CT0219 Homo | 1 | 1 | upregulate stage |
| 411560 | AW851186 | | gb: IL3-CT0220-150200-071-H05 CT0220 Homo | 2.8 | 0.17 | upregulate stage |
| 411568 | BE144593 | | gb: MR0-HT0167-141199-002-f04 HT0167 Homo | 1 | 1 | upregulate stage |
| 411571 | AA122393 | Hs.70811 | hypothetical protein FLJ20516 | 3.55 | 0.14 | upregulate stage |
| 411605 | AW006831 | Hs.20479 | ESTs | 9.6 | 0.08 | upregulate stage |
| 411626 | AW793453 | Hs.71109 | KIAA1229 protein | 1 | 1 | upregulate stage |
| 411630 | U42349 | Hs.71119 | Putative prostate cancer tumor suppresso | 4.1 | 0.11 | upregulate stage |
| 411643 | AI924519 | Hs.192570 | Homo sapiens cDNA: FLJ22028 fis, clone H | 1 | 0.28 | upregulate stage |
| 411653 | AF070578 | Hs.71168 | Homo sapiens clone 24674 mRNA sequence | 8.9 | 0.08 | upregulate stage |
| 411727 | AW858443 | | gb: CM0-CT0341-260100-160-f10 CT0341 Homo | 1 | 1 | upregulate stage |
| 411771 | AW993247 | | gb: RC2-BN0033-180200-014-h09 BN0033 Homo | 2.6 | 0.14 | upregulate stage |
| 411787 | AW863568 | | gb: MR3-SN0010-240300-102-c10 SN0010 Homo | 1 | 1 | upregulate stage |
| 411788 | AW897793 | | gb: CM1-NN0063-280400-203-f07 NN0063 Homo | 3.7 | 0.15 | upregulate stage |
| 411826 | AW947946 | | gb: PM0-MT0011-240300-001-a09 MT0011 Homo | 3.25 | 0.13 | upregulate stage |
| 411835 | U29343 | Hs.72550 | hyaluronan-mediated motility receptor (R | 1 | 1 | upregulate stage |
| 411860 | T89420 | | gb: yd98f04.s1 Soares fetal liver spleen | 1 | 0.22 | upregulate stage |
| 411874 | AA096106 | Hs.20403 | ESTs | 5.75 | 0.11 | upregulate stage |
| 411917 | AW876360 | Hs.3592 | Homo sapiens cDNA: FLJ22555 fis, clone H | 1 | 0.33 | upregulate stage |
| 411928 | AA888624 | Hs.19121 | adaptor-related protein complex 2, alpha | 4.75 | 0.12 | upregulate stage |
| 411932 | AW876548 | | gb: RC3-PT0028-190100-012-h02 PT0028 Homo | 1 | 0.38 | upregulate stage |
| 411943 | BE502436 | Hs.7962 | ESTs, Weakly similar to putative [C. eleg | 3.82 | 0.23 | upregulate stage |
| 411945 | AL033527 | Hs.92137 | v-myc avian myelocytomatosis viral oncog | 4.65 | 0.15 | upregulate stage |
| 411991 | X58822 | Hs.73010 | interferon, omega 1 | 2.45 | 0.14 | upregulate stage |
| 412040 | D86519 | Hs.73086 | neuropeptide Y receptor Y6 (pseudogene) | 4.6 | 0.14 | upregulate stage |
| 412088 | AI689496 | Hs.108932 | ESTs | 2.82 | 0.18 | upregulate stage |
| 412134 | AW895560 | | gb: QV4-NN0038-270400-187-g08 NN0038 Homo | 6.4 | 0.1 | upregulate stage |
| 412140 | AA219691 | Hs.73625 | RAB6 interacting, kinesin-like (rabkines | 17.05 | 0.04 | upregulate stage |
| 412231 | AW902491 | Hs.289088 | heat shock 90 kD protein 1, alpha | 1 | 0.91 | upregulate stage |
| 412296 | AW936233 | | gb: QV0-DT0020-090200-107-a06 DT0020 Homo | 1 | 1 | upregulate stage |
| 412327 | AW937355 | | gb: QV3-DT0043-211299-044-a06 DT0043 Homo | 1 | 1 | upregulate stage |
| 412357 | AW939537 | | gb: QV1-DT0072-110200-066-f05 DT0072 Homo | 1 | 0.24 | upregulate stage |
| 412359 | AW837985 | | gb: QV3-LT0048-140200-083-e05 LT0048 Homo | 1 | 0.41 | upregulate stage |
| 412367 | AW945964 | | gb: QV0-ET0001-050500-228-e09 ET0001 Homo | 1 | 0.22 | upregulate stage |
| 412529 | BE271224 | Hs.266273 | Homo sapiens cDNA FLJ13346 fis, clone OV | 4.45 | 0.14 | upregulate stage |
| 412530 | AA766268 | Hs.266273 | Homo sapiens cDNA FLJ13346 fis, clone OV | 9.3 | 0.08 | upregulate stage |
| 412537 | AL031778 | Hs.797 | nuclear transcription factor Y, alpha | 4.25 | 0.14 | upregulate stage |
| 412547 | W27161 | | gb: 23a12 Human retina cDNA randomly prim | 1 | 1 | upregulate stage |
| 412559 | T31474 | | gb: EST33147 Human Embryo Homo sapiens cD | 1 | 0.26 | upregulate stage |
| 412636 | NM_004415 | Hs.74316 | desmoplakin (DPI, DPII) | 12.05 | 0.05 | upregulate stage |
| 412648 | AA115211 | Hs.69658 | EST | 1 | 0.28 | upregulate stage |
| 412668 | AA456195 | Hs.10056 | ESTs | 10.75 | 0.07 | upregulate stage |
| 412671 | AW977734 | | gb: EST389963 MAGE resequences, MAGO Homo | 2.65 | 0.3 | upregulate stage |
| 412673 | AL042957 | Hs.31845 | ESTs | 4.6 | 0.11 | upregulate stage |
| 412723 | AA648459 | Hs.179912 | ESTs | 2.55 | 0.11 | upregulate stage |
| 412739 | AA116018 | Hs.271809 | Homo sapiens cDNA: FLJ22406 fis, clone H | 1.6 | 0.24 | upregulate stage |
| 412744 | N31101 | | gb: yx52a03.r1 Soares melanocyte 2NbHM Ho | 2 | 0.23 | upregulate stage |
| 412778 | AA120882 | Hs.159244 | ESTs | 1 | 1 | upregulate stage |
| 412811 | H06382 | Hs.21400 | ESTs | 1 | 0.49 | upregulate stage |
| 412838 | D61870 | | gb: HUM218F11B Clontech human aorta polyA | 1 | 0.34 | upregulate stage |
| 412854 | BE004149 | Hs.31161 | ESTs | 1 | 1 | upregulate stage |
| 413075 | D59828 | Hs.70953 | ESTs | 1 | 0.77 | upregulate stage |
| 413109 | AW389845 | Hs.110855 | ESTs | 3.93 | 0.1 | upregulate stage |
| 413117 | BE066107 | Hs.138484 | ESTs, Weakly similar to ALU1_HUMAN ALU S | 1 | 0.22 | upregulate stage |
| 413119 | BE065941 | | gb: RC3-BT0319-100100-012-d12 BT0319 Homo | 1 | 0.87 | upregulate stage |
| 413141 | BE166323 | | gb: QV4-HT0492-270100-086-e12 HT0492 Homo | 5.45 | 0.12 | upregulate stage |
| 413219 | AA878200 | Hs.118727 | Homo sapiens cDNA FLJ13692 fis, clone PL | 2.54 | 0.19 | upregulate stage |
| 413228 | AA127518 | Hs.195870 | ESTs | 1 | 1 | upregulate stage |
| 413273 | U75679 | Hs.75257 | Hairpin binding protein, histone | 5.05 | 0.11 | upregulate stage |
| 413278 | BE563085 | Hs.833 | interferon-stimulated protein, 15 kDa | 1.45 | 0.5 | upregulate stage |
| 413294 | BE144034 | | gb: MR0-HT0165-191199-004-a02 HT0165 Homo | 1 | 1 | upregulate stage |
| 413324 | V00571 | Hs.75294 | corticotropin releasing hormone | 6.95 | 0.03 | upregulate stage |
| 413342 | AA128535 | | gb: zl24e04.r1 Soares_pregnant_uterus_NbH | 1 | 1 | upregulate stage |
| 413430 | R22479 | Hs.24650 | Homo sapiens cDNA FLJ13047 fis, clone NT | 3 | 0.18 | upregulate stage |
| 413707 | BE158679 | | gb: CM0-HT0395-280100-169-c04 HT0395 Homo | 1 | 0.28 | upregulate stage |
| 413743 | BE161004 | | gb: PM0-HT0425-170100-002-h03 HT0425 Homo | 1 | 1 | upregulate stage |
| 413753 | U17760 | Hs.301103 | Human DNA sequence from clone 272L16 on | 22.7 | 0.03 | upregulate stage |
| 413786 | AW613780 | Hs.13500 | ESTs | 9.9 | 0.07 | upregulate stage |
| 413792 | BE166924 | | gb: CM4-HT0501-240300-519-f01 HT0501 Homo | 1 | 1 | upregulate stage |
| 413804 | T64682 | | gb: yc48b02.r1 Stratagene liver (937224) | 0.99 | 0.75 | upregulate stage |
| 413833 | Z15005 | Hs.75573 | centromere protein E (312 kD) | 2.55 | 0.17 | upregulate stage |
| 413854 | BE174300 | Hs.44581 | heat shock protein hsp70-related protein | 1.25 | 0.24 | upregulate stage |
| 413918 | AW015898 | Hs.71245 | ESTs | 4 | 0.11 | upregulate stage |
| 413968 | AW500374 | Hs.64056 | ESTs | 10.85 | 0.07 | upregulate stage |
| 414091 | T83742 | | gb: yd67g02.s1 Soares fetal liver spleen | 8.9 | 0.1 | upregulate stage |
| 414099 | U11313 | Hs.75760 | sterol carrier protein 2 | 10.3 | 0.06 | upregulate stage |
| 414116 | AA587370 | Hs.71584 | ESTs | 1 | 1 | upregulate stage |
| 414127 | AI431863 | Hs.135270 | ESTs | 2.85 | 0.13 | upregulate stage |
| 414169 | AA136169 | Hs.149335 | ESTs | 8.95 | 0.09 | upregulate stage |
| 414275 | AW970254 | Hs.889 | Charot-Leyden crystal protein | 7.05 | 0.05 | upregulate stage |
| 414304 | AI621276 | Hs.165998 | DKFZP564M2423 protein | 1 | 0.24 | upregulate stage |
| 414338 | N80751 | Hs.301471 | ESTs | 10.3 | 0.08 | upregulate stage |
| 414447 | AA147549 | Hs.109909 | ESTs | 3.4 | 0.16 | upregulate stage |
| 414494 | AA768491 | Hs.6783 | Homo sapiens cDNA: FLJ22724 fis, clone H | 3.4 | 0.18 | upregulate stage |
| 414520 | AA148806 | Hs.204046 | ESTs | 1 | 0.21 | upregulate stage |
| 414569 | AF109298 | Hs.118258 | prostate cancer associated protein 1 | 3.1 | 0.18 | upregulate stage |
| 414575 | H11257 | Hs.295233 | ESTs | 3.1 | 0.15 | upregulate stage |
| 414597 | H67472 | Hs.34274 | ESTs | 4.6 | 0.11 | upregulate stage |
| 414643 | H46177 | Hs.119316 | ESTs | 1 | 0.28 | upregulate stage |
| 414658 | X58528 | Hs.76781 | ATP-binding cassette, sub-family D (ALD) | 7.75 | 0.08 | upregulate stage |
| 414661 | T97401 | Hs.21929 | ESTs | 1 | 0.26 | upregulate stage |
| 414683 | S78296 | Hs.76888 | internexin neuronal intermediate filamen | 2.72 | 0.25 | upregulate stage |
| 414735 | BE468016 | Hs.281904 | ESTs | 1 | 0.38 | upregulate stage |
| 414737 | AI160386 | Hs.125087 | ESTs | 5.5 | 0.1 | upregulate stage |
| 414747 | U30872 | Hs.77204 | centromere protein F (350/400 kD, mitosin | 3.19 | 0.24 | upregulate stage |
| 414774 | X02419 | Hs.77274 | plasminogen activator, urokinase | 1.45 | 0.49 | upregulate stage |
| 414783 | AW069569 | Hs.75839 | zinc finger protein 6 (CMPX1) | 4.65 | 0.13 | upregulate stage |
| 414799 | AI752416 | Hs.77326 | insulin-like growth factor binding prote | 1.7 | 0.46 | upregulate stage |
| 414833 | T07114 | | gb: EST05003 Fetal brain, Stratagene (cat | 4.5 | 0.13 | upregulate stage |
| 414883 | AA926960 | Hs.77550 | CDC28 protein kinase 1 | 3.36 | 0.22 | upregulate stage |
| 414885 | AA157531 | Hs.269276 | ESTs | 2.7 | 0.21 | upregulate stage |
| 414918 | AI219207 | Hs.72222 | Homo sapiens cDNA FLJ13459 fis, clone PL | 0.87 | 0.69 | upregulate stage |
| 414985 | C17372 | | gb: C17372 Clontech human aorta polyA + mR | 1 | 0.42 | upregulate stage |
| 415025 | AW207091 | Hs.72307 | ESTs | 5.3 | 0.06 | upregulate stage |
| 415033 | D31476 | Hs.301448 | Homo sapiens cDNA FLJ12152 fis, clone MA | 1 | 1 | upregulate stage |
| 415060 | AJ223810 | Hs.43213 | ESTs, Weakly similar to IEFS_HUMAN TRANS | 6.05 | 0.1 | upregulate stage |
| 415068 | Z19448 | Hs.131887 | ESTs, Weakly similar to ORF YNL227c [S.c | 4.5 | 0.13 | upregulate stage |
| 415095 | D59592 | Hs.34745 | ESTs | 1 | 0.44 | upregulate stage |
| 415099 | AI492170 | Hs.77917 | ubiquitin carboxyl-terminal esterase L3 | 2.27 | 0.29 | upregulate stage |
| 415104 | D60076 | | gb: HUM084E10A Clontech human fetal brain | 3.95 | 0.13 | upregulate stage |
| 415114 | D60468 | | gb: HUM111A06B Clontech human fetal brain | 2.05 | 0.2 | upregulate stage |
| 415138 | C18356 | Hs.78045 | tissue factor pathway inhibitor 2 | 5.8 | 0.05 | upregulate stage |
| 415139 | AW975942 | Hs.48524 | ESTs | 1.15 | 0.21 | upregulate stage |
| 415148 | Z36953 | Hs.48527 | ESTs | 2.5 | 0.2 | upregulate stage |
| 415153 | C03508 | Hs.7000 | ESTs | 8.95 | 0.09 | upregulate stage |
| 415178 | D80503 | | gb: HUM080A02B Human fetal brain (TFujiwa | 1 | 0.15 | upregulate stage |
| 415217 | H23983 | Hs.26922 | ESTs | 1 | 0.31 | upregulate stage |
| 415227 | AW821113 | Hs.72402 | ESTs | 6.3 | 0.11 | upregulate stage |
| 415238 | R37780 | Hs.21422 | ESTs | 1 | 1 | upregulate stage |
| 415241 | F02208 | Hs.27214 | ESTs | 1 | 1 | upregulate stage |
| 415295 | R41450 | Hs.6546 | ESTs | 1 | 0.63 | upregulate stage |
| 415296 | F05086 | | gb: HSC01A011 normalized infant brain cDN | 5.65 | 0.1 | upregulate stage |
| 415327 | H22769 | Hs.1861 | membrane protein, palmitoylated 1 (55 kD) | 8.15 | 0.09 | upregulate stage |
| 415330 | Z44693 | Hs.21422 | ESTs | 3 | 0.2 | upregulate stage |
| 415336 | T77664 | Hs.78362 | Human clone 23839 mRNA sequence | 1 | 0.87 | upregulate stage |
| 415337 | Z44881 | Hs.9012 | ESTs | 8.8 | 0.07 | upregulate stage |
| 415352 | F06565 | | gb: HSC1CG051 normalized infant brain cDN | 1 | 1 | upregulate stage |
| 415364 | F06771 | | gb: HSC1KD031 normalized infant brain cDN | 1 | 1 | upregulate stage |
| 415371 | R15239 | | gb: yf89b02.r1 Soares infant brain 1NIB H | 5.1 | 0.13 | upregulate stage |
| 415412 | F08049 | Hs.52132 | ESTs | 4.25 | 0.16 | upregulate stage |
| 415451 | H19415 | Hs.268720 | ESTs, Moderately similar to ALU1_HUMAN A | 4.1 | 0.15 | upregulate stage |
| 415462 | R52692 | Hs.12698 | ESTs | 4.65 | 0.11 | upregulate stage |
| 415496 | R37637 | Hs.12286 | ESTs | 5.4 | 0.13 | upregulate stage |
| 415509 | R40000 | Hs.91968 | ESTs | 1 | 0.44 | upregulate stage |
| 415511 | AI732617 | Hs.182362 | ESTs | 9.3 | 0.03 | upregulate stage |
| 415542 | R13474 | Hs.290263 | ESTs | 9.7 | 0.08 | upregulate stage |
| 415569 | Z43930 | | gb: HSC1OH121 normalized infant brain cDN | 1 | 0.74 | upregulate stage |
| 415600 | F12664 | | gb: HSC3CG021 normalized infant brain cDN | 1 | 0.43 | upregulate stage |
| 415616 | F12945 | Hs.12294 | ESTs | 1 | 1 | upregulate stage |
| 415626 | Z43847 | | gb: HSC1MC051 normalized infant brain cDN | 1 | 1 | upregulate stage |
| 415635 | F13168 | | gb: HSC3JF101 normalized infant brain cDN | 1 | 0.26 | upregulate stage |
| 415750 | AA167712 | | gb: zq39g08.s1 Stratagene hNT neuron (937 | 1 | 0.83 | upregulate stage |
| 415786 | AW419196 | Hs.257924 | Homo sapiens cDNA FLJ13782 fis, clone PL | 9 | 0.08 | upregulate stage |
| 415788 | AW628686 | Hs.78851 | KIAA0217 protein | 5.2 | 0.11 | upregulate stage |
| 415790 | R23574 | Hs.23545 | ESTs | 1 | 1 | upregulate stage |
| 415799 | AA653718 | Hs.225841 | DKFZP434D193 protein | 4.25 | 0.12 | upregulate stage |
| 415837 | H05279 | Hs.21758 | ESTs | 1 | 0.57 | upregulate stage |
| 415857 | AA866115 | Hs.301646 | Homo sapiens cDNA FLJ11381 fis, clone HE | 8.05 | 0.07 | upregulate stage |
| 415906 | AI751357 | Hs.288741 | Homo sapiens cDNA: FLJ22256 fis, clone H | 12.2 | 0.06 | upregulate stage |
| 415947 | U04045 | Hs.78934 | mutS (E. coli) homolog 2 (colon cancer, | 12.2 | 0.06 | upregulate stage |
| 415948 | AA262226 | | gb: zs24h06.r1 NCI_CGAP_GCB1 Homo sapiens | 1 | 1 | upregulate stage |
| 415979 | H16427 | Hs.271501 | ESTs | 4.85 | 0.13 | upregulate stage |
| 415989 | AI267700 | Hs.111128 | ESTs | 4.45 | 0.08 | upregulate stage |
| 416018 | AW138239 | Hs.78977 | proprotein convertase subtilisin/kexin t | 1 | 1 | upregulate stage |
| 416052 | R12816 | Hs.21164 | ESTs | 1.45 | 0.24 | upregulate stage |
| 416053 | H16359 | Hs.130648 | ESTs | 4.35 | 0.14 | upregulate stage |
| 416061 | R45516 | Hs.26119 | ESTs | 1 | 1 | upregulate stage |
| 416065 | BE267931 | Hs.78996 | proliferating cell nuclear antigen | 4.72 | 0.17 | upregulate stage |
| 416097 | BE387371 | Hs.301304 | Homo sapiens cDNA: FLJ21017 fis, clone C | 5.75 | 0.11 | upregulate stage |
| 416111 | AA033813 | Hs.79018 | chromatin assembly factor 1, subunit A ( | 8.4 | 0.09 | upregulate stage |
| 416135 | AW473656 | Hs.45119 | ESTs | 2.29 | 0.2 | upregulate stage |
| 416155 | AI807264 | Hs.205442 | ESTs, Weakly similar to AF117610 1 inner | 5.1 | 0.13 | upregulate stage |
| 416173 | R52782 | | gb: yg99d09.r1 Soares infant brain 1NIB H | 3.7 | 0.12 | upregulate stage |
| 416195 | AW131940 | Hs.104030 | ESTs | 1.1 | 0.16 | upregulate stage |
| 416196 | W51955 | Hs.73372 | ESTs | 3.25 | 0.14 | upregulate stage |
| 416203 | H27794 | Hs.269055 | ESTs | 1 | 0.32 | upregulate stage |
| 416209 | AA236776 | Hs.79078 | MAD2 (mitotic arrest deficient, yeast, h | 4.15 | 0.12 | upregulate stage |
| 416226 | N55342 | Hs.34372 | ESTs | 2.35 | 0.21 | upregulate stage |
| 416239 | AL038450 | Hs.48948 | ESTs | 4.05 | 0.14 | upregulate stage |
| 416241 | N52639 | Hs.32683 | ESTs | 5 | 0.09 | upregulate stage |
| 416254 | H51703 | Hs.13640 | ESTs | 1 | 0.95 | upregulate stage |
| 416269 | AA177138 | Hs.161671 | ESTs | 4.07 | 0.2 | upregulate stage |
| 416276 | U41060 | Hs.79136 | LIV-1 protein, estrogen regulated | 1.84 | 0.45 | upregulate stage |
| 416280 | H44180 | Ns. 181789 | ESTs | 1 | 1 | upregulate stage |
| 416309 | R84694 | Hs.79194 | cAMP responsive element binding protein | 9.35 | 0.08 | upregulate stage |
| 416324 | H47983 | Hs.1870 | phenylalanine hydroxylase | 5.15 | 0.13 | upregulate stage |
| 416332 | H91284 | Hs.244461 | ESTs | 1 | 1 | upregulate stage |
| 416343 | H49213 | | gb: yq19e04.r1 Soares fetal liver spleen | 1 | 1 | upregulate stage |
| 416353 | T77127 | Hs.191297 | ESTs, Moderately similar to ALU6_HUMAN A | 1.46 | 0.59 | upregulate stage |
| 416395 | R94575 | | gb: yt73e10.s1 Soares fetal liver spleen | 9.2 | 0.09 | upregulate stage |
| 416437 | N48990 | Hs.37204 | ESTs | 4.15 | 0.12 | upregulate stage |
| 416476 | H58137 | Hs.268639 | ESTs | 1 | 0.22 | upregulate stage |
| 416537 | T99086 | Hs.144904 | nuclear receptor co-repressor 1 | 5.45 | 0.12 | upregulate stage |
| 416539 | Y07909 | Hs.79368 | epithelial membrane protein 1 | 9.45 | 0.09 | upregulate stage |
| 416575 | W02414 | Hs.38383 | ESTs | 4.95 | 0.1 | upregulate stage |
| 416624 | H69044 | | gb: yr77h05.s1 Soares fetal liver spleen | 1 | 0.22 | upregulate stage |
| 416644 | H70701 | Hs.269135 | ESTs | 5.65 | 0.12 | upregulate stage |
| 416658 | U03272 | Hs.79432 | fibrillin 2 (congenital contractural ara | 9.65 | 0.05 | upregulate stage |
| 416682 | R99700 | Hs.36152 | ESTs | 1 | 0.25 | upregulate stage |
| 416690 | H84078 | Hs.108551 | ESTs | 5.35 | 0.13 | upregulate stage |
| 416709 | R99369 | Hs.283108 | hemoglobin, gamma G | 5.4 | 0.13 | upregulate stage |
| 416712 | N68576 | Hs.81602 | ESTs | 1 | 0.25 | upregulate stage |
| 416715 | H79460 | Hs.271722 | ESTs, Weakly similar to ALU1_HUMAN ALU S | 1 | 0.32 | upregulate stage |
| 416731 | T58115 | Hs.10336 | ESTs | 1 | 0.4 | upregulate stage |
| 416734 | H81213 | Hs.14825 | ESTs | 3.8 | 0.16 | upregulate stage |
| 416735 | R11275 | Hs.194485 | ESTs | 11.5 | 0.06 | upregulate stage |
| 416738 | N29218 | Hs.40290 | ESTs | 1 | 0.42 | upregulate stage |
| 416856 | N27833 | Hs.269028 | ESTs | 2.6 | 0.22 | upregulate stage |
| 416883 | AW140128 | Hs.184902 | ESTs | 11.3 | 0.07 | upregulate stage |
| 416923 | N32498 | Hs.42829 | ESTs | 1 | 0.61 | upregulate stage |
| 416936 | N21352 | Hs.42987 | ESTs, Weakly similar to ORF2 [M. musculus | 1 | 1 | upregulate stage |
| 417018 | M16038 | Hs.80887 | v-yes-1 Yamaguchi sarcoma viral related | 11.3 | 0.05 | upregulate stage |
| 417079 | U65590 | Hs.81134 | interleukin 1 receptor antagonist | 5.04 | 0.15 | upregulate stage |
| 417134 | N51220 | Hs.269068 | ESTs | 1 | 0.24 | upregulate stage |
| 417185 | NM_002484 | Hs.81469 | nucleotide binding protein 1 (E. coli Min | 1.98 | 0.32 | upregulate stage |
| 417218 | AA005247 | Hs.285754 | met proto-oncogene (hepatocyte growth fa | 2.95 | 0.21 | upregulate stage |
| 417265 | AL121369 | Hs.281117 | ESTs | 1 | 0.3 | upregulate stage |
| 417283 | N62840 | Hs.48648 | ESTs | 1.05 | 0.27 | upregulate stage |
| 417308 | H60720 | Hs.81892 | KIAA0101 gene product | 9.2 | 0.09 | upregulate stage |
| 417320 | AA195667 | Hs.287324 | ESTs | 2.8 | 0.16 | upregulate stage |
| 417396 | T98987 | | gb: ye66f02.r1 Soares fetal liver spleen | 1 | 1 | upregulate stage |
| 417404 | NM_007350 | Hs.82101 | pleckstrin homology-like domain, family | 2.75 | 0.09 | upregulate stage |
| 417409 | BE272506 | Hs.82109 | syndecan 1 | 1.92 | 0.44 | upregulate stage |
| 417448 | AA203135 | Hs.130186 | ESTs | 6.45 | 0.1 | upregulate stage |
| 417453 | H73183 | Hs.129885 | ESTs, Moderately similar to unnamed prot | 4.65 | 0.13 | upregulate stage |
| 417515 | L24203 | Hs.82237 | ataxia-telangiectasia group D-associated | 1.59 | 0.49 | upregulate stage |
| 417540 | AA203600 | Hs.152250 | ESTs | 1 | 1 | upregulate stage |
| 417576 | AA339449 | Hs.82285 | phosphoribosylglycinamide formyltransfer | 5.65 | 0.1 | upregulate stage |
| 417581 | R26968 | Hs.24104 | ESTs, Weakly similar to ALU7_HUMAN ALU S | 9.15 | 0.09 | upregulate stage |
| 417596 | R07343 | Hs.226823 | ESTs | 4.35 | 0.14 | upregulate stage |
| 417599 | AA204688 | Hs.136201 | ESTs, Weakly similar to ALU7_HUMAN ALU S | 0.94 | 0.9 | upregulate stage |
| 417620 | R02530 | Hs.191198 | ESTs | 9.1 | 0.07 | upregulate stage |
| 417638 | R12490 | Hs.189779 | ESTs | 1 | 0.32 | upregulate stage |
| 417650 | T05870 | Hs.100640 | ESTs | 1 | 0.22 | upregulate stage |
| 417715 | AW969587 | Hs.86366 | ESTs | 6.31 | 0.09 | upregulate stage |
| 417720 | AA205625 | Hs.208067 | ESTs | 4.65 | 0.11 | upregulate stage |
| 417742 | R64719 | | gb: EST22d11 WATM1 Homo sapiens cDNA clon | 4.15 | 0.13 | upregulate stage |
| 417750 | AI267720 | Hs.260523 | neuroblastoma RAS viral (v-ras) oncogene | 9.98 | 0.08 | upregulate stage |
| 417780 | Z43482 | Hs.82772 | collagen, type XI, alpha 1 | 2.3 | 0.14 | upregulate stage |
| 417789 | R50978 | Hs.267054 | ESTs | 1.05 | 0.19 | upregulate stage |
| 417791 | AW965339 | Hs.111471 | ESTs | 5.35 | 0.1 | upregulate stage |
| 417850 | AA215724 | Hs.82741 | primase, polypeptide 1 (49 kD) | 1 | 1 | upregulate stage |
| 417898 | AA826198 | Hs.291851 | ESTs | 2.15 | 0.21 | upregulate stage |
| 417975 | AA641836 | Hs.30085 | Homo sapiens cDNA: FLJ23186 fis, clone L | 3.7 | 0.13 | upregulate stage |
| 418004 | U37519 | Hs.87539 | aldehyde dehydrogenase 8 | 1.57 | 0.5 | upregulate stage |
| 418007 | M13509 | Hs.83169 | matrix metalloproteinase 1 (interstitial | 17.9 | 0.02 | upregulate stage |
| 418027 | AB037807 | Hs.83293 | hypothetical protein | 6.6 | 0.09 | upregulate stage |
| 418030 | BE207573 | Hs.83321 | neuromedin B | 12.2 | 0.04 | upregulate stage |
| 418068 | AW971155 | Hs.293902 | ESTs, Weakly similar to prolyl 4-hydroxy | 4.26 | 0.14 | upregulate stage |
| 418113 | AI272141 | Hs.83484 | SRY (sex determining region Y)-box 4 | 5.21 | 0.15 | upregulate stage |
| 418134 | AA397769 | Hs.86617 | ESTs | 1 | 0.3 | upregulate stage |
| 418153 | R13696 | Hs.112830 | ESTs | 1 | 0.3 | upregulate stage |
| 418180 | BE618087 | Hs.83724 | Human clone 23773 mRNA sequence | 8.79 | 0.09 | upregulate stage |
| 418201 | AA214345 | Hs.98445 | Homo sapiens cDNA: FLJ21652 fis, clone C | 3.75 | 0.13 | upregulate stage |
| 418203 | X54942 | Hs.83758 | CDC28 protein kinase 2 | 13.85 | 0.04 | upregulate stage |
| 418216 | AA662240 | Hs.283099 | AF15q14 protein | 9.75 | 0.07 | upregulate stage |
| 418236 | AW994005 | Hs.172572 | hypothetical protein FLJ20093 | 10.75 | 0.05 | upregulate stage |
| 418250 | U29926 | Hs.83918 | adenosine monophosphate deaminase (isofo | 6.25 | 0.12 | upregulate stage |
| 418259 | AA215404 | Hs.137289 | ESTs | 11.5 | 0.07 | upregulate stage |
| 418268 | AA810599 | Hs.86643 | ESTs | 1 | 0.43 | upregulate stage |
| 418296 | C01566 | Hs.86671 | ESTs | 1 | 0.45 | upregulate stage |
| 418372 | AA311833 | Hs.84318 | replication protein A1 (70 kD) | 9.04 | 0.08 | upregulate stage |
| 418379 | AA218940 | Hs.137516 | fidgetin-like 1 | 3.25 | 0.15 | upregulate stage |
| 418422 | AW440068 | Hs.59425 | Homo sapiens cDNA: FLJ23323 fis, clone H | 8.95 | 0.1 | upregulate stage |
| 418454 | AA315308 | | gb: EST187095 Colon carcinoma (HCC) cell | 2.5 | 0.15 | upregulate stage |
| 418462 | BE001596 | Hs.85266 | integrin, beta 4 | 1.33 | 0.59 | upregulate stage |
| 418469 | U34879 | Hs.85279 | hydroxysteroid (17-beta) dehydrogenase 1 | 1.21 | 0.71 | upregulate stage |
| 418478 | U38945 | Hs.1174 | cyclin-dependent kinase inhibitor 2A (me | 2.69 | 0.23 | upregulate stage |
| 418480 | AA223929 | Hs.86902 | ESTs | 1 | 1 | upregulate stage |
| 418498 | T78248 | | gh: yd79f05.r1 Soares fetal liver spleen | 1 | 0.47 | upregulate stage |
| 418516 | NM_006218 | Hs.85701 | phosphoinositide-3-kinase, catalytic, al | 5.4 | 0.14 | upregulate stage |
| 418546 | AA224827 | | gb: nc32g04.s1 NCI_CGAP_Pr2 Homo sapiens | 2.72 | 0.23 | upregulate stage |
| 418573 | AA225188 | | gb: nc21h04.r1 NCI_CGAP_Pr1 Homo sapiens | 9.95 | 0.07 | upregulate stage |
| 418577 | AA225247 | Hs.269300 | ESTs, Weakly similar to B34087 hypotheti | 1 | 0.77 | upregulate stage |
| 418578 | U92459 | Hs.86204 | glutamate receptor, metabotropic 8 | 1 | 1 | upregulate stage |
| 418590 | AI732672 | Hs.252507 | ESTs | 1 | 0.59 | upregulate stage |
| 418592 | X99226 | Hs.284153 | Fanconi anemia, complementation group A | 4.75 | 0.13 | upregulate stage |
| 418612 | AB037788 | Hs.224961 | cleavage and polyadenylation specific fa | 1 | 0.23 | upregulate stage |
| 418624 | AI734080 | Hs.104211 | ESTs | 7.95 | 0.09 | upregulate stage |
| 418661 | NM_001949 | Hs.1189 | Human mRNA for KIAA0075 gene, partial cd | 3 | 0.15 | upregulate stage |
| 418663 | AK001100 | Hs.87013 | Homo sapiens cDNA FLJ10238 fis, clone HE | 17.2 | 0.04 | upregulate stage |
| 418675 | AW299723 | Hs.87223 | bone morphogenetic protein receptor, typ | 1 | 1 | upregulate stage |
| 418686 | Z36830 | Hs.87268 | annexin A8 | 2.11 | 0.3 | upregulate stage |
| 418687 | R61650 | Hs.22581 | ESTs | 6.75 | 0.07 | upregulate stage |
| 418693 | AI750878 | Hs.87409 | thrombospondin 1 | 4.5 | 0.08 | upregulate stage |
| 418704 | AA227235 | Hs.83286 | ESTs | 1 | 0.33 | upregulate stage |
| 418712 | Z42183 | | gb: HSC0BF041 normalized infant brain cDN | 1 | 0.91 | upregulate stage |
| 418717 | AI334430 | Hs.86984 | ESTs | 4.7 | 0.12 | upregulate stage |
| 418723 | AA504428 | Hs.10487 | ESTs, Weakly similar to Weak similarity | 5.85 | 0.1 | upregulate stage |
| 418738 | AW388633 | Hs.6682 | ESTs | 3.6 | 0.09 | upregulate stage |
| 418752 | AL133556 | Hs.88144 | hypothetical protein FLJ12476 | 1 | 1 | upregulate stage |
| 418757 | AI864193 | Hs.169728 | Homo sapiens cDNA FLJ13150 fis, clone NT | 9.15 | 0.09 | upregulate stage |
| 418844 | M62982 | Hs.1200 | arachidonate 12-lipoxygenase | 9.25 | 0.08 | upregulate stage |
| 418867 | D31771 | Hs.89404 | msh (Drosophila) homo box homolog 2 | 1.83 | 0.43 | upregulate stage |
| 418876 | AA740616 | Hs.293874 | ESTs | 11.3 | 0.06 | upregulate stage |
| 418903 | AW969665 | Hs.154848 | ESTs | 1 | 1 | upregulate stage |
| 418915 | AI474778 | Hs.118977 | ESTs | 4.75 | 0.12 | upregulate stage |
| 418939 | AW630803 | Hs.89497 | lamin B1 | 2.6 | 0.13 | upregulate stage |
| 418945 | BE246762 | Hs.89499 | arachidonate 5-lipoxygenase | 1.45 | 0.53 | upregulate stage |
| 418976 | AA933082 | Hs.126883 | ESTs | 1 | 0.23 | upregulate stage |
| 419059 | T86216 | | gb: yd84a05.r1 Soares fetal liver spleen | 1 | 0.38 | upregulate stage |
| 419078 | M93119 | Hs.89584 | insulinoma-associated 1 | 1.25 | 0.18 | upregulate stage |
| 419121 | AA374372 | Hs.89626 | parathyroid hormone-like hormone | 1 | 1 | upregulate stage |
| 419169 | AW851980 | Hs.262346 | ESTs, Weakly similar to ORF2: function u | 1.59 | 0.3 | upregulate stage |
| 419183 | U60669 | Hs.89663 | cytochrome P450, subfamily XXIV (vitamin | 3.55 | 0.05 | upregulate stage |
| 419218 | AI248073 | Hs.188723 | ESTs, Weakly similar to ALU1_HUMAN ALU S | 1 | 0.27 | upregulate stage |
| 419226 | AI342491 | Hs.87413 | ESTs | 1 | 0.37 | upregulate stage |
| 419235 | AW470411 | Hs.288433 | neurotrimin | 11.9 | 0.07 | upregulate stage |
| 419286 | AA236005 | Hs.221303 | ESTs | 4.85 | 0.14 | upregulate stage |
| 419327 | AA521504 | Hs.190179 | ESTs | 1 | 1 | upregulate stage |
| 419355 | AA428520 | Hs.90061 | progesterone binding protein | 10.6 | 0.06 | upregulate stage |
| 419359 | AL043202 | Hs.90073 | chromosome segregation 1 (yeast homolog) | 1.84 | 0.47 | upregulate stage |
| 419413 | AA237040 | Hs.87589 | ESTs | 1 | 1 | upregulate stage |
| 419436 | AA991639 | Hs.15036 | ESTs, Highly similar to AF161358 1 HSPC0 | 6.6 | 0.1 | upregulate stage |
| 419452 | U33635 | Hs.90572 | PTK7 protein tyrosine kinase 7 | 1.31 | 0.64 | upregulate stage |
| 419472 | AW978038 | | gb: EST390147 MAGE resequences, MAGO Homo | 1 | 1 | upregulate stage |
| 419475 | AA243420 | Hs.87648 | ESTs | 1.1 | 0.24 | upregulate stage |
| 419477 | AA826279 | | gb: od03g07.s1 NCI_CGAP_GCB1 Homo sapiens | 1 | 0.56 | upregulate stage |
| 419484 | AA243474 | Hs.272128 | Homo sapiens cDNA FLJ13901 fis, clone TH | 1 | 0.22 | upregulate stage |
| 419506 | N20912 | Hs.42369 | ESTs | 1 | 1 | upregulate stage |
| 419554 | AI732138 | Hs.104318 | ESTs | 1 | 0.5 | upregulate stage |
| 419569 | AI971651 | Hs.91143 | jagged 1 (Alagille syndrome) | 1 | 0.91 | upregulate stage |
| 419594 | AA013051 | Hs.91417 | topoisomerase (DNA) II binding protein | 8.1 | 0.08 | upregulate stage |
| 419651 | NM_007023 | Hs.91971 | cAMP-regulated guanine nucleotide exchan | 1 | 1 | upregulate stage |
| 419666 | NM_014810 | Hs.92200 | KIAA0480 gene product | 5.2 | 0.12 | upregulate stage |
| 419737 | H24185 | Hs.92918 | hypothetical protein | 11.7 | 0.07 | upregulate stage |
| 419743 | AW408762 | Hs.127478 | ESTs | 6.1 | 0.09 | upregulate stage |
| 419752 | AA249573 | Hs.152618 | ESTs | 1.8 | 0.17 | upregulate stage |
| 419769 | H27374 | Hs.103483 | ESTs | 1 | 0.36 | upregulate stage |
| 419805 | AW966945 | | gb: EST379019 MAGE resequences, MAGJ Homo | 1 | 0.34 | upregulate stage |
| 419807 | R77402 | | gb: yi75f11.s1 Soares placenta Nb2HP Homo | 1 | 0.67 | upregulate stage |
| 419831 | AW448930 | Hs.5415 | ESTs | 7.05 | 0.1 | upregulate stage |
| 419833 | AA251131 | Hs.220697 | ESTs | 1.25 | 0.53 | upregulate stage |
| 419834 | AA251139 | | gb: zs03g12.s1 NCI_CGAP_GCB1 Homo sapiens | 1 | 1 | upregulate stage |
| 419923 | AW081455 | Hs.120219 | ESTs | 5.89 | 013 | upregulate stage |
| 419945 | AW290975 | Hs.118923 | ESTs | 1 | 0.24 | upregulate stage |
| 419962 | AA830111 | Hs.291917 | ESTs | 1 | 1 | upregulate stage |
| 419970 | AW612022 | Hs.263271 | ESTs | 9.15 | 0.09 | upregulate stage |
| 419986 | AI345455 | Hs.78915 | GA-binding protein transcription factor, | 3.05 | 0.17 | upregulate stage |
| 419998 | AA252691 | | gb: zs26d09.r1 NCI_CGAP_GCB1 Homo sapiens | 1 | 0.47 | upregulate stage |
| 420016 | AW016908 | Hs.88025 | ESTs | 1 | 0.8 | upregulate stage |
| 420047 | AI478658 | Hs.94631 | brefeldin A-inhibited guanine nucleotide | 4.8 | 0.11 | upregulate stage |
| 420076 | AA827860 | Hs.293717 | ESTs | 5.35 | 0.12 | upregulate stage |
| 420111 | AA255652 | | gb: zs21h11.r1 NCI_CGAP_GCB1 Homo sapiens | 5.3 | 0.11 | upregulate stage |
| 420145 | AA809860 | Hs.256284 | ESTs | 1 | 1 | upregulate stage |
| 420159 | AI572490 | Hs.99785 | Homo sapiens cDNA: FLJ21245 fis, clone C | 14.8 | 0.04 | upregulate stage |
| 420161 | AI683069 | Hs.175319 | ESTs | 4.7 | 0.11 | upregulate stage |
| 420184 | AA188408 | Hs.95665 | hypothetical protein | 4.35 | 0.15 | upregulate stage |
| 420226 | AA773709 | Hs.152818 | ubiquitin specific protease 8 | 3.1 | 0.16 | upregulate stage |
| 420230 | AL034344 | Hs.298020 | Homo sapiens cDNA FLJ11796 fis, clone HE | 10.35 | 0.06 | upregulate stage |
| 420236 | AA256763 | Hs.291111 | ESTs | 4.45 | 0.14 | upregulate stage |
| 420270 | AA257990 | | gh: zs35h07.r1 NCI_CGAP_GCB1 Homo sapiens | 10.05 | 0.08 | upregulate stage |
| 420297 | AI628272 | Hs.88323 | ESTs | 9.45 | 0.09 | upregulate stage |
| 420344 | BE463721 | Hs.97101 | putative G protein-coupled receptor | 11.7 | 0.05 | upregulate stage |
| 420392 | AI242930 | Hs.97393 | KIAA0328 protein | 1.7 | 0.22 | upregulate stage |
| 420413 | AW971624 | Hs.120605 | ESTs | 1 | 1 | upregulate stage |
| 420445 | AA262213 | Hs.193514 | ESTs | 1 | 1 | upregulate stage |
| 420471 | AA262452 | Hs.192268 | ESTs | 3.95 | 0.13 | upregulate stage |
| 420479 | AW183695 | Hs.186572 | ESTs | 4.95 | 0.12 | upregulate stage |
| 420493 | AI635113 | Hs.270366 | Homo sapiens mRNA; cDNA DKFZp564H0616 (f | 4.4 | 0.15 | upregulate stage |
| 420552 | AK000492 | Hs.98806 | hypothetical protein | 11.55 | 0.06 | upregulate stage |
| 420572 | AL035593 | Hs.99016 | Human DNA sequence from clone 310J6 on c | 1.35 | 0.22 | upregulate stage |
| 420643 | W87731 | | gb: zh65g10.r1 Soares_fetal_liver_spleen_{—} | 1.25 | 0.25 | upregulate stage |
| 420650 | AA455706 | Hs.44581 | heat shock protein hsp70-related protein | 7.3 | 0.09 | upregulate stage |
| 420654 | AA279091 | Hs.104420 | ESTs | 1 | 0.27 | upregulate stage |
| 420655 | R74405 | Hs.300886 | ESTs | 1 | 1 | upregulate stage |
| 420717 | AA284447 | Hs.271887 | ESTs | 9 | 0.09 | upregulate stage |
| 420734 | AW972872 | Hs.293736 | ESTs | 5.2 | 0.13 | upregulate stage |
| 420756 | AA411800 | Hs.189900 | ESTs | 1 | 1 | upregulate stage |
| 420789 | AI670057 | Hs.199882 | ESTs | 8.85 | 0.06 | upregulate stage |
| 420802 | U22376 | Hs.1334 | v-myb avian myeloblastosis viral oncogen | 4.6 | 0.12 | upregulate stage |
| 420851 | AA281062 | Hs.250734 | ESTs | 8.35 | 0.08 | upregulate stage |
| 420880 | AI809621 | Hs.105620 | ESTs | 1 | 1 | upregulate stage |
| 420923 | AF097021 | Hs.273321 | differentially expressed in hematopoieti | 10.4 | 0.03 | upregulate stage |
| 420928 | AA281809 | | gb: zt10e01.r1 NCI_CGAP_GCB1 Homo sapiens | 1 | 1 | upregulate stage |
| 420936 | AA456112 | Hs.99410 | ESTs | 8.71 | 0.07 | upregulate stage |
| 420947 | AA491044 | Hs.47196 | ESTs | 1 | 0.38 | upregulate stage |
| 421017 | AW979181 | Hs.293221 | ESTs, Weakly similar to ALU1_HUMAN ALU S | 1 | 1 | upregulate stage |
| 421064 | AI245432 | Hs.101382 | tumor necrosis factor, alpha-induced pro | 1.26 | 0.62 | upregulate stage |
| 421070 | AA283185 | Hs.19327 | ESTs | 2.2 | 0.14 | upregulate stage |
| 421100 | AW351839 | Hs.124660 | Homo sapiens cDNA: FLJ21763 fis, clone C | 1.69 | 0.28 | upregulate stage |
| 421102 | AI470093 | Hs.89217 | ESTs | 2.65 | 0.19 | upregulate stage |
| 421103 | AI625835 | Hs.27104 | ESTs | 6 | 0.1 | upregulate stage |
| 421114 | AW975051 | Hs.293156 | ESTs | 4.7 | 0.12 | upregulate stage |
| 421118 | AI471925 | Hs.89257 | ESTs | 1 | 0.39 | upregulate stage |
| 421155 | H87879 | Hs.102267 | lysyl oxidase | 1.15 | 0.18 | upregulate stage |
| 421159 | AW978316 | Hs.136649 | ESTs | 1 | 0.44 | upregulate stage |
| 421187 | NM_014721 | Hs.102471 | KIAA0680 gene product | 5.7 | 0.11 | upregulate stage |
| 421218 | NM_000499 | Hs.72912 | cytochrome P450, subfamily I (aromatic c | 0.07 | 2.55 | upregulate stage |
| 421221 | AW276914 | Hs.300877 | ESTs | 8.75 | 0.07 | upregulate stage |
| 421229 | AI056590 | Hs.7086 | Homo sapiens cDNA: FLJ23000 fis, clone L | 1.64 | 0.49 | upregulate stage |
| 421261 | AA600853 | Hs.98133 | ESTs | 10.9 | 0.07 | upregulate stage |
| 421262 | AA286746 | Hs.9343 | Homo sapiens cDNA FLJ14265 fis, clone PL | 2.65 | 0.16 | upregulate stage |
| 421278 | AI367919 | Hs.99691 | ESTs | 1 | 0.56 | upregulate stage |
| 421280 | AA811804 | | gb: ob39a05.s1 NCI_CGAP_GCB1 Homo sapiens | 1 | 0.34 | upregulate stage |
| 421282 | AA286914 | Hs.183299 | ESTs | 9 | 0.08 | upregulate stage |
| 421306 | AA806207 | Hs.125889 | ESTs | 1 | 0.95 | upregulate stage |
| 421308 | AA687322 | Hs.192843 | ESTs | 2.85 | 0.15 | upregulate stage |
| 421373 | AA808229 | Hs.167771 | ESTs | 2.45 | 0.14 | upregulate stage |
| 421379 | Y15221 | Hs.103982 | small inducible cytokine subfamily B (Cy | 1.6 | 0.26 | upregulate stage |
| 421381 | AA361752 | | gb: EST71314 T-cell lymphoma Homo sapiens | 5.05 | 0.09 | upregulate stage |
| 421418 | AA806639 | | gb: obB8g05.s1 NCI_CGAP_GCB1 Homo sapiens | 6.55 | 0.1 | upregulate stage |
| 421433 | AI829192 | Hs.134805 | ESTs | 9.9 | 0.07 | upregulate stage |
| 421451 | AA291377 | Hs.50831 | ESTs | 11.9 | 0.06 | upregulate stage |
| 421491 | H99999 | Hs.42736 | ESTs | 3 | 0.2 | upregulate stage |
| 421493 | BE300341 | Hs.104925 | ectodermal-neural cortex (with BTB-like | 2.51 | 0.32 | upregulate stage |
| 421559 | NM_014720 | Hs.105751 | Ste20-related serine/threonine kinase | 9 | 0.09 | upregulate stage |
| 421577 | BE465451 | Hs.105925 | single-minded (Drosophila) homolog 1 | 5.75 | 0.12 | upregulate stage |
| 421673 | H54384 | Hs.36892 | ESTs | 1 | 1 | upregulate stage |
| 421685 | AF189723 | Hs.106778 | ATPase, Ca++ transporting, type 2C, memb | 9.45 | 0.07 | upregulate stage |
| 421708 | AW754341 | | gb: CM0-CT0341-181299-130-h12 CT0341 Homo | 1 | 0.47 | upregulate stage |
| 421733 | AL119671 | Hs.1420 | fibroblast growth factor receptor 3 (ach | 1.97 | 0.33 | upregulate stage |
| 421838 | AW881089 | Hs.108806 | Homo sapiens mRNA; cDNA DKFZp566M0947 (f | 7.05 | 0.1 | upregulate stage |
| 421869 | AB003592 | Hs.109050 | contactin 6 | 1 | 1 | upregulate stage |
| 421925 | S80310 | Hs.109620 | acidic epididymal glycoprotein-like 1 | 1 | 1 | upregulate stage |
| 421948 | L42583 | Hs.111758 | keratin 6A | 51.9 | 0.01 | upregulate stage |
| 421958 | AA357185 | Hs.109918 | ras homolog gene family, member H | 10.17 | 0.07 | upregulate stage |
| 421991 | NM_014918 | Hs.110488 | KIAA0990 protein | 4.5 | 0.17 | upregulate stage |
| 422026 | U80736 | Hs.110826 | trinucleotide repeat containing 9 | 6.5 | 0.08 | upregulate stage |
| 422072 | AB018255 | Hs.111138 | KIAA0712 gene product | 9.2 | 0.08 | upregulate stage |
| 422094 | AF129535 | Hs.272027 | F-box only protein 5 | 6.95 | 0.09 | upregulate stage |
| 422158 | L10343 | Hs.112341 | protease inhibitor 3, skin-derived (SKAL | 1.66 | 0.17 | upregulate stage |
| 422168 | AA586894 | Hs.112408 | S100 calcium-binding protein A7 (psorias | 3.96 | 0.1 | upregulate stage |
| 422182 | AL043892 | Hs.180582 | Homo sapiens cDNA: FLJ21836 fis, clone H | 2.8 | 0.16 | upregulate stage |
| 422204 | AA339015 | | gb: EST44247 Fetal brain I Homo sapiens c | 1 | 1 | upregulate stage |
| 422261 | AA307595 | Hs.119908 | nucleolar protein NOP5/NOP58 | 1 | 1 | upregulate stage |
| 422271 | AB038995 | Hs.114159 | RAB-8b protein | 5.04 | 0.16 | upregulate stage |
| 422278 | AF072873 | Hs.114218 | frizzled (Drosophila) homolog 6 | 3.46 | 0.24 | upregulate stage |
| 422282 | AF019225 | Hs.114309 | apolipoprotein L | 4.54 | 0.14 | upregulate stage |
| 422322 | AB022192 | Hs.115240 | peroxisome biogenesis factor 13 | 1 | 0.53 | upregulate stage |
| 422330 | D30783 | Hs.115263 | epiregulin | 4.45 | 0.06 | upregulate stage |
| 422342 | AA309272 | | gb: EST180209 Liver, hepatocellular carci | 2.25 | 0.19 | upregulate stage |
| 422406 | AF025441 | Hs.116206 | Opa-interacting protein 5 | 9.5 | 0.07 | upregulate stage |
| 422487 | AJ010901 | Hs.198267 | mucin 4, tracheobronchial | 7.35 | 0.04 | upregulate stage |
| 422491 | AA338548 | Hs.117546 | neuronatin | 0.64 | 1.24 | upregulate stage |
| 422504 | AA311407 | | gb: EST182167 Jurkat T-cells V Homo sapie | 3.6 | 0.11 | upregulate stage |
| 422505 | AL120862 | Hs.124165 | ESTs | 2.8 | 0.14 | upregulate stage |
| 422508 | AJ000327 | Hs.117852 | ATP-binding cassette, sub-family D (ALD) | 5.25 | 0.14 | upregulate stage |
| 422530 | AW972300 | Hs.118110 | bone marrow stromal cell antigen 2 | 2.57 | 0.31 | upregulate stage |
| 422540 | AI050751 | Hs.22895 | Homo sapiens cDNA: FLJ23548 fis, clone L | 1 | 0.59 | upregulate stage |
| 422588 | AA312730 | | gb: EST183651 Monocytes, stimulated II Ho | 3 | 0.14 | upregulate stage |
| 422678 | AA247778 | Hs.119155 | Homo sapiens mRNA; cDNA DKFZp434B249 (fr | 10.7 | 0.07 | upregulate stage |
| 422762 | AL031320 | Hs.119976 | Human DNA sequence from clone RP1-20N2 o | 5.1 | 0.13 | upregulate stage |
| 422809 | AK001379 | Hs.121028 | hypothetical protein FLJ10549 | 5.94 | 0.1 | upregulate stage |
| 422823 | D89974 | Hs.121102 | vanin 2 | 10 | 0.07 | upregulate stage |
| 422892 | AA988176 | Hs.121553 | hypothetical protein FLJ20641 | 1 | 0.27 | upregulate stage |
| 422938 | NM_001809 | Hs.1594 | centromere protein A (17 kD) | 7.2 | 0.08 | upregulate stage |
| 422964 | AW439476 | Hs.256895 | ESTs | 11.75 | 0.07 | upregulate stage |
| 422981 | AF026445 | Hs.122752 | TATA box binding protein (TBP)-associate | 3.05 | 0.14 | upregulate stage |
| 423001 | AA320014 | Hs.208603 | ESTs | 9.1 | 0.09 | upregulate stage |
| 423090 | BE387529 | Hs.123536 | melanoma antigen, family E, 1, cancer/te | 1 | 0.49 | upregulate stage |
| 423100 | AA323114 | | gb: EST25873 Cerebellum II Homo sapiens c | 1 | 1 | upregulate stage |
| 423121 | AW864848 | | gb: PM2-SN0018-290300-003-c09 SN0018 Homo | 2.8 | 0.19 | upregulate stage |
| 423156 | AA131493 | Hs.124752 | fibroblast growth factor 12B | 1 | 0.27 | upregulate stage |
| 423198 | M81933 | Hs.1634 | cell division cycle 25A | 8.95 | 0.07 | upregulate stage |
| 423217 | NM_000094 | Hs.1640 | collagen, type VII, alpha 1 (epidermolys | 1.2 | 0.57 | upregulate stage |
| 423262 | NM_005479 | Hs.126057 | frequently rearranged in advanced T-cell | 9.75 | 0.07 | upregulate stage |
| 423296 | AW957193 | Hs.3327 | Homo sapiens cDNA: FLJ22219 fis, clone H | 4.95 | 0.12 | upregulate stage |
| 423309 | BE006775 | Hs.126782 | sushi-repeat protein | 1.58 | 0.34 | upregulate stage |
| 423347 | AI660412 | Hs.234557 | ESTs | 1 | 0.45 | upregulate stage |
| 423359 | NM_014170 | Hs.127496 | HSPC135 protein | 1 | 0.69 | upregulate stage |
| 423368 | AA364195 | | gb: EST75015 Pineal gland II Homo sapiens | 1 | 0.95 | upregulate stage |
| 423389 | AI471609 | Hs.54347 | ESTs | 3.95 | 0.14 | upregulate stage |
| 423430 | AF112481 | Hs.128501 | RAD54, S. cerivisiae, homolog of, B | 1.62 | 0.43 | upregulate stage |
| 423441 | R68649 | Hs.278359 | absent in melanoma 1 like | 6.25 | 0.1 | upregulate stage |
| 423453 | AW450737 | Hs.128791 | CGI-09 protein | 8.45 | 0.09 | upregulate stage |
| 423500 | AF020763 | Hs.129705 | clone 1900 unknown protein | 1 | 0.8 | upregulate stage |
| 423578 | AW960454 | Hs.222830 | ESTs | 11.94 | 0.07 | upregulate stage |
| 423629 | AW021173 | Hs.18612 | Homo sapiens cDNA: FLJ21909 fis, clone H | 3.4 | 0.17 | upregulate stage |
| 423632 | AA328824 | | gb: EST32358 Embryo, 12 week I Homo sapie | 1 | 0.71 | upregulate stage |
| 423642 | AW452650 | Hs.157148 | Homo sapiens cDNA FLJ11883 fis, clone HE | 8.35 | 0.1 | upregulate stage |
| 423644 | AA4329048 | | gb: EST32875 Embryo, 12 week I Homo sapie | 1 | 0.43 | upregulate stage |
| 423648 | AK000456 | Hs.130546 | hypothetical protein FLJ20449 | 10.4 | 0.07 | upregulate stage |
| 423651 | AF192913 | Hs.130683 | zinc finger protein 180 (HHZ168) | 5.75 | 0.1 | upregulate stage |
| 423654 | AI674253 | Hs.35828 | ESTs | 3.15 | 0.18 | upregulate stage |
| 423673 | BE003054 | Hs.1695 | matrix metalloproteinase 12 (macrophage | 29.7 | 0.02 | upregulate stage |
| 423725 | AJ403108 | Hs.132127 | hypothetical protein LOC57822 | 4.1 | 0.16 | upregulate stage |
| 423745 | AI809797 | Hs.43222 | ESTs | 1 | 0.5 | upregulate stage |
| 423748 | AI149048 | Hs.30211 | hypothetical protein FLJ22313 | 4.25 | 0.13 | upregulate stage |
| 423753 | Y11312 | Hs.132463 | phosphoinositide-3-kinase, class 2, beta | 1.18 | 0.71 | upregulate stage |
| 423758 | AA338153 | Hs.82124 | laminin, beta 1 | 1 | 1 | upregulate stage |
| 423774 | L39064 | Hs.1702 | interleukin 9 receptor | 3.08 | 0.15 | upregulate stage |
| 423818 | AA332439 | | gb: EST36554 Embryo, 8 week I Homo sapien | 1 | 0.38 | upregulate stage |
| 423827 | AI472828 | Hs.172625 | ESTs | 1 | 0.43 | upregulate stage |
| 423837 | AW937063 | | gb: PM3-DT0037-231299-001-g11 DT0037 Homo | 1.44 | 0.55 | upregulate stage |
| 423912 | BE091233 | | gb: PM0-BT0726-300300-001-H07 BT0726 Homo | 1 | 1 | upregulate stage |
| 423938 | AL049328 | Hs.135642 | Homo sapiens mRNA; cDNA DKFZp564E026 (fr | 1 | 1 | upregulate stage |
| 423942 | AF209704 | Hs.135723 | glycolipid transfer protein | 11.65 | 0.05 | upregulate stage |
| 423944 | T91433 | Hs.128291 | phesphodiesterase 10A | 1 | 0.45 | upregulate stage |
| 423946 | AL137344 | Hs.135892 | Homo sapiens mRNA; cDNA DKFZp76101311 (f | 1 | 1 | upregulate stage |
| 423956 | W28203 | Hs.136169 | Homo sapiens clone 25215 mRNA sequence, | 5.35 | 0.09 | upregulate stage |
| 424006 | AF054815 | Hs.137548 | CD84 antigen (leukocyte antigen) | 8.8 | 0.06 | upregulate stage |
| 424008 | R02740 | Hs.137555 | putative chemokine receptor; GTP-binding | 3.14 | 0.19 | upregulate stage |
| 424012 | AW368377 | Hs.137569 | tumor protein 63 kDa with strong homolog | 2.6 | 0.26 | upregulate stage |
| 424073 | U03493 | Hs.138959 | gap junction protein, alpha 7, 45 kD (con | 1.8 | 0.22 | upregulate stage |
| 424075 | AI807320 | Hs.227630 | RE1-silencing transcription factor | 9.1 | 0.06 | upregulate stage |
| 424087 | N69333 | Hs.21638 | ESTs | 1 | 1 | upregulate stage |
| 424193 | AK002005 | Hs.142868 | Homo sapiens cDNA FLJ11143 fis, clone PL | 1 | 0.23 | upregulate stage |
| 424353 | AA339646 | | gb: EST44755 Fetal brain I Home sapiens c | 1 | 1 | upregulate stage |
| 424364 | AW383226 | Hs.201189 | ESTs, Weakly similar to DRPLA [H. sapiens | 2.18 | 0.33 | upregulate stage |
| 424406 | D54120 | Hs.146409 | wingless-type MMTV integration site fami | 2.05 | 0.17 | upregulate stage |
| 424420 | BE614743 | Hs.146688 | prostaglandin E synthase | 1.19 | 0.67 | upregulate stage |
| 424425 | AB031480 | Hs.146824 | SPR1 protein | 1.42 | 0.54 | upregulate stage |
| 424486 | BE002477 | Hs.278714 | chloride intracellelar channel 6 | 1 | 0.27 | upregulate stage |
| 424490 | AJ278016 | Hs.55565 | ankyrin repeat domain 3 | 2.02 | 0.39 | upregulate stage |
| 424492 | AI133482 | Hs.165210 | ESTs | 3.15 | 0.14 | upregulate stage |
| 424505 | AA446131 | Hs.124918 | Homo sapiens cDNA FLJ13186 fis, close NT | 11.55 | 0.05 | upregulate stage |
| 424513 | BE385864 | Hs.149894 | mitochondrial translational initiation f | 2.65 | 0.23 | upregulate stage |
| 424575 | AL110217 | Hs.150751 | DKFZP572C163 protein | 1 | 1 | upregulate stage |
| 424583 | AF017445 | Hs.150926 | fucose-1-phosphate guanylyltransferase | 1.8 | 0.26 | upregulate stage |
| 424589 | AW854298 | | gb: RC3-CT0254-100500-211-c03 CT0254 Homo | 0.85 | 0.94 | upregulate stage |
| 424602 | AK002055 | Hs.301129 | Homo sapiens clone 23859 mRNA sequence | 2.85 | 0.2 | upregulate stage |
| 424625 | AW904466 | Hs.151310 | PDZ domain protein (Drosophila inaD-like | 1 | 0.4 | upregulate stage |
| 424629 | M90656 | Hs.151393 | glutamate-cysteine ligase, catalytic sub | 1.41 | 0.52 | upregulate stage |
| 424643 | AF241850 | Hs.151428 | ret finger protein 2 | 9.75 | 0.07 | upregulate stage |
| 424649 | BE242035 | Hs.151461 | embryonic ectoderm development | 5.85 | 0.13 | upregulate stage |
| 424653 | AW977534 | Hs.151469 | calcium/calmodulin-dependent serine prot | 1 | 0.56 | upregulate stage |
| 424670 | W61215 | Hs.116651 | epithelial V-like antigen 1 | 1.42 | 0.52 | upregulate stage |
| 424690 | BE538356 | Hs.151777 | Human translation initiation factor elF- | 4.3 | 0.1 | upregulate stage |
| 424701 | NM_005923 | Hs.151988 | mitogen-activated protein kinase kinase | 3.85 | 0.11 | upregulate stage |
| 424702 | AF250237 | Hs.152009 | G protein-coupled receptor 85 | 1 | 1 | upregulate stage |
| 424717 | H03754 | Hs.152213 | wingless-type MMTV integration site fami | 4.05 | 0.09 | upregulate stage |
| 424735 | U31875 | Hs.152677 | Homo sapiens cDNA FLJ20338 fis, clone HE | 1.04 | 0.59 | upregulate stage |
| 424736 | AF230877 | Hs.152701 | microtubule-interacting protein that ass | 1.11 | 0.65 | upregulate stage |
| 424749 | NM_002451 | Hs.152817 | methylthioadenosine phosphorylase | 1 | 1 | upregulate stage |
| 424834 | AK001432 | Hs.153408 | Homo sapiens cDNA FLJ10570 fis, clone NT | 18.5 | 0.03 | upregulate stage |
| 424841 | AI280215 | Hs.96885 | ESTs | 1 | 1 | upregulate stage |
| 424860 | W60828 | Hs.153529 | Homo sapiens clone 24540 mRNA sequence | 1 | 1 | upregulate stage |
| 424878 | H57111 | Hs.221132 | ESTs | 9.45 | 0.07 | upregulate stage |
| 424879 | AA348013 | Hs.159354 | ESTs | 10.7 | 0.07 | upregulate stage |
| 424888 | AA348126 | Hs.24882 | ESTs | 2.8 | 0.21 | upregulate stage |
| 424905 | NM_002497 | Hs.153704 | NIMA (never in mitosis gene a)-related k | 7.75 | 0.07 | upregulate stage |
| 424930 | AA885344 | Hs.96910 | ESTs | 1.45 | 0.38 | upregulate stage |
| 424948 | AA348810 | Hs.190503 | ESTs | 3.2 | 0.12 | upregulate stage |
| 424951 | AW964082 | | gb: EST376155 MAGE resequences, MAGH Homo | 8.75 | 0.09 | upregulate stage |
| 424993 | F07625 | | gb: HSC2CF021 normalized infant brain cDN | 1 | 1 | upregulate stage |
| 425020 | U09368 | Hs.154205 | zinc finger protein 140 (clone pHZ-39) | 1 | 1 | upregulate stage |
| 425024 | R39235 | Hs.12407 | ESTs | 2.65 | 0.13 | upregulate stage |
| 425057 | AA826434 | Hs.96944 | ESTs | 1 | 0.22 | upregulate stage |
| 425068 | AL048716 | Hs.154387 | KIAA0103 gene product | 9.5 | 0.07 | upregulate stage |
| 425081 | X74794 | Hs.154443 | minichromosome maintenance deficient (S. | 1.66 | 0.52 | upregulate stage |
| 425191 | AF052146 | Hs.155085 | Homo sapiens clone 24653 mRNA sequence | 1 | 0.32 | upregulate stage |
| 425216 | U81504 | Hs.155172 | adaptor-related protein complex 3, beta | 7.05 | 0.1 | upregulate stage |
| 425234 | AW152225 | Hs.165909 | ESTs | 19.7 | 0.04 | upregulate stage |
| 425239 | BE567924 | Hs.155244 | pre-mRNA splicing factor similar to S. c | 1 | 0.69 | upregulate stage |
| 425289 | AW139342 | Hs.155530 | interferon, gamma-inducible protein 16 | 10.45 | 0.05 | upregulate stage |
| 425304 | AA463844 | Hs.31339 | fibroblast growth factor 11 | 1.57 | 0.51 | upregulate stage |
| 425316 | AA354977 | Hs.191565 | ESTs, Moderately similar to NSD1 protein | 5.05 | 0.12 | upregulate stage |
| 425322 | U63630 | Hs.155637 | protein kinase, DNA-activated, catalytic | 14.7 | 0.05 | upregulate stage |
| 425362 | AA355936 | | gb: EST64410 Jurkat T-cells VI Homo sapie | 1 | 1 | upregulate stage |
| 425397 | J04088 | Hs.156346 | topoisomerase (DNA) II alpha (170 kD) | 8.24 | 0.09 | upregulate stage |
| 425403 | AL023753 | Hs.156406 | Human DNA sequence from clone 1198H6 on | 1 | 0.22 | upregulate stage |
| 425415 | M13903 | Hs.157091 | involucrin | 1.19 | 0.55 | upregulate stage |
| 425420 | BE536911 | Hs.234545 | ESTs, Weakly similar to AF155135 1 novel | 2.85 | 0.13 | upregulate stage |
| 425463 | AK000740 | Hs.157986 | hypothetical protein FLJ20733 | 9 | 0.07 | upregulate stage |
| 425465 | L18964 | Hs.1904 | protein kinase C, iota | 9.6 | 0.07 | upregulate stage |
| 425467 | R16484 | Hs.190075 | ESTs | 1 | 0.83 | upregulate stage |
| 425492 | AL021918 | Hs.158174 | zinc finger protein 184 (Kruppel-like) | 3.2 | 0.15 | upregulate stage |
| 425607 | U09860 | Hs.158333 | protease, serine, 7 (enterokinase) | 1 | 1 | upregulate stage |
| 425608 | AA360486 | Hs.92448 | ESTs | 4.7 | 0.14 | upregulate stage |
| 425614 | AI334963 | Hs.156256 | ESTs | 2.65 | 0.14 | upregulate stage |
| 425641 | D79758 | Hs.14355 | Homo sapiens cDNA FLJ13207 fis, clone NT | 4.86 | 0.1 | upregulate stage |
| 425660 | AA521184 | Hs.105504 | ESTs | 1 | 0.31 | upregulate stage |
| 425665 | AK001050 | Hs.159066 | hypothetical protein FLJ10188 | 1.25 | 0.19 | upregulate stage |
| 425672 | AA361483 | | gb: EST70790 T-cell lymphoma Homo sapiens | 1 | 1 | upregulate stage |
| 425707 | AF115402 | Hs.11713 | E74-like factor 5 (ets domain transcript | 3.96 | 0.13 | upregulate stage |
| 425726 | AF085808 | Hs.159330 | uroplakin 3 | 0.92 | 0.79 | upregulate stage |
| 425742 | AJ001454 | Hs.159425 | testican 3 | 1 | 1 | upregulate stage |
| 425785 | T27017 | Hs.159528 | Homo sapiens clone 24400 mRNA sequence | 1 | 0.39 | upregulate stage |
| 425811 | AL039104 | Hs.159557 | karyopherin alpha 2 (RAG cohort 1, impor | 1.89 | 0.44 | upregulate stage |
| 425843 | BE313280 | Hs.159627 | death associated protein 3 | 3.1 | 0.15 | upregulate stage |
| 425852 | AK001504 | Hs.159651 | death receptor 6 | 1.72 | 0.47 | upregulate stage |
| 425883 | AL137708 | Hs.161031 | Homo sapiens mRNA; cDNA DKFZp434K0322 (f | 0.95 | 0.68 | upregulate stage |
| 426010 | AA136563 | Hs.1975 | Homo sapiens cDNA: FLJ21007 fis, clone C | 1 | 0.34 | upregulate stage |
| 426028 | NM_001110 | Hs.172028 | a disintegrin and metalloproteinase doma | 14.3 | 0.04 | upregulate stage |
| 426101 | AL049987 | Hs.166361 | Homo sapiens mRNA; cDNA DKFZp564F112 (fr | 11.75 | 0.05 | upregulate stage |
| 426108 | AA622037 | Hs.166468 | programmed cell death 5 | 3.23 | 0.18 | upregulate stage |
| 426115 | H08895 | Hs.166733 | leucyl/cystinyl aminopeptidase | 1 | 0.32 | upregulate stage |
| 426168 | NM_003152 | Hs.167503 | signal transducer and activator of trans | 1.97 | 0.4 | upregulate stage |
| 426257 | AL137201 | Hs.168625 | KIAA0979 protein | 1 | 0.29 | upregulate stage |
| 426261 | AW242243 | Hs.168670 | peroxisomal farnesylated protein | 2.8 | 0.16 | upregulate stage |
| 426283 | NM_003937 | Hs.169139 | kynureninase (L-kynurenine hydrolase) | 14.75 | 0.04 | upregulate stage |
| 426451 | AI908165 | Hs.169946 | GATA-binding protein 3 | 3.05 | 0.28 | upregulate stage |
| 426462 | U59111 | Hs.169993 | dermatan sulphate proteoglycan 3 | 1 | 0.36 | upregulate stage |
| 426490 | NM_001621 | Hs.170087 | aryl hydrocarbon receptor | 14.17 | 0.05 | upregulate stage |
| 426514 | BE616633 | Hs.301122 | bone morphogenetic protein 7 (osteogenic | 1.15 | 0.47 | upregulate stage |
| 426561 | AA381437 | | gb: EST94514 Activated T-cells I Homo sap | 5.65 | 0.11 | upregulate stage |
| 426711 | AA383471 | Hs.180669 | conserved gene amplified in osteosarcoma | 11.05 | 0.05 | upregulate stage |
| 426731 | AW303411 | Hs.130332 | ESTs | 2.4 | 0.21 | upregulate stage |
| 426759 | AI590401 | Hs.21213 | ESTs | 9.5 | 0.06 | upregulate stage |
| 426786 | AA319798 | Hs.172247 | eukaryotic translation elongation factor | 9.25 | 0.09 | upregulate stage |
| 426788 | U66615 | Hs.172280 | SWI/SNF related, matrix associated, acti | 5.63 | 0.14 | upregulate stage |
| 426818 | AA554827 | Hs.124841 | ESTs, Weakly similar to ALU5_HUMAN ALU S | 9 | 0.08 | upregulate stage |
| 426824 | D87717 | Hs.172652 | KIAA0013 gene product | 1 | 0.87 | upregulate stage |
| 426827 | AW067805 | Hs.172665 | methylenetetrahydrofolate dehydrogenase | 8.95 | 0.09 | upregulate stage |
| 426921 | AA037145 | Hs.172865 | cleavage stimulates factor, 3′ pre-RNA, | 1 | 0.19 | upregulate stage |
| 426935 | NM_000088 | Hs.172928 | collagen, type I, alpha 1 | 1.15 | 0.72 | upregulate stage |
| 426997 | BE620738 | Hs.173125 | peptidylprolyl isomerase F (cyclophilin | 11.06 | 0.06 | upregulate stage |
| 427071 | AA397958 | Hs.192719 | ESTs | 5.75 | 0.08 | upregulate stage |
| 427126 | AA620613 | Hs.191827 | ESTs | 2.55 | 0.18 | upregulate stage |
| 427134 | AA398409 | Hs.173561 | EST | 3.4 | 0.18 | upregulate stage |
| 427142 | AA398510 | Hs.133148 | ESTs | 1 | 0.25 | upregulate stage |
| 427259 | AA400096 | | gb: zu69f07.s1 Soares_testis_NHT Home sap | 1 | 0.22 | upregulate stage |
| 427308 | D26067 | Hs.174905 | KIAA0033 protein | 5.9 | 0.1 | upregulate stage |
| 427315 | AA179949 | Hs.175563 | Home sapiens mRNA; cDNA DKFZp564N0763 (7 | 6.12 | 0.11 | upregulate stage |
| 427356 | AW023482 | Hs.97849 | ESTs | 2.7 | 0.13 | upregulate stage |
| 427370 | AI243615 | Hs.97740 | ESTs | 3.6 | 0.14 | upregulate stage |
| 427376 | AA401533 | Hs.19440 | ESTs | 2.1 | 0.16 | upregulate stage |
| 427387 | BE244966 | Hs.177584 | 3-oxoacid CoA transferase | 1 | 0.39 | upregulate stage |
| 427470 | AW999924 | Hs.178357 | Homo sapiens cDNA FLJ13657 fis, clone PL | 3.4 | 0.16 | upregulate stage |
| 427519 | AW085233 | Hs.180696 | ESTs | 8.23 | 0.1 | upregulate stage |
| 427521 | AW973352 | Hs.299056 | ESTs | 7.75 | 0.1 | upregulate stage |
| 427528 | AU077143 | Hs.179565 | minichromosome maintenance deficient (S. | 5.7 | 0.15 | upregulate stage |
| 427566 | AI743515 | | gb: wf72b08.x2 Soares_NFL_T_GBC_S1 Homo s | 1 | 1 | upregulate stage |
| 427581 | NM_014788 | Hs.179703 | KIAA0129 gene product | 11.45 | 0.06 | upregulate stage |
| 427585 | D31152 | Hs.179729 | collagen, type X, alpha 1 (Schmid metaph | 3.25 | 0.16 | upregulate stage |
| 427603 | AI090838 | Hs.98006 | ESTs | 1 | 1 | upregulate stage |
| 427646 | AI678042 | Hs.271953 | ESTs | 5.7 | 0.11 | upregulate stage |
| 427652 | AI673025 | Hs.43874 | ESTs | 1 | 0.34 | upregulate stage |
| 427742 | AA411880 | Hs.190888 | ESTs | 2.4 | 0.16 | upregulate stage |
| 427814 | W28383 | Hs.180900 | Williams-Beuren syndrome chromosome regi | 9.13 | 0.08 | upregulate stage |
| 427839 | AA608823 | Hs.98244 | ESTs | 1.9 | 0.19 | upregulate stage |
| 427878 | C05766 | Hs.181022 | CGI-07 protein | 4.1 | 0.14 | upregulate stage |
| 427922 | AK001934 | Hs.181112 | HSPC126 protein | 2.6 | 0.19 | upregulate stage |
| 427933 | AW974643 | Hs.190571 | ESTs | 4.55 | 0.14 | upregulate stage |
| 427934 | AA810541 | Hs.291866 | ESTs | 1 | 1 | upregulate stage |
| 427944 | AA417878 | Hs.48401 | ESTs, Weakly similar to ALU8_HUMAN ALU S | 6.15 | 0.1 | upregulate stage |
| 427961 | AW293165 | Hs.143134 | ESTs | 4.85 | 0.11 | upregulate stage |
| 427986 | N45214 | Hs.282387 | Homo sapiens cDNA: FLJ21837 fis, clone H | 3.55 | 0.13 | upregulate stage |
| 428003 | AL110200 | Hs.181384 | Homo sapiens mRNA; cDNA DKFZp586B0922 (f | 1.45 | 0.36 | upregulate stage |
| 428004 | AA449563 | Hs.300270 | ESTs | 3.95 | 0.12 | upregulate stage |
| 428010 | AA806554 | Hs.185375 | ESTs | 1 | 0.38 | upregulate stage |
| 428057 | AI343641 | Hs.185798 | ESTs | 10.1 | 0.06 | upregulate stage |
| 428058 | AI821625 | Hs.191602 | ESTs | 1 | 0.5 | upregulate stage |
| 428071 | AF212848 | Hs.182339 | ets homologous factor | 6.4 | 0.09 | upregulate stage |
| 428182 | BE386042 | Hs.293317 | ESTs, Weakly similar to JM27 [H. sapiens ] | 1 | 0.23 | upregulate stage |
| 428192 | AA424051 | | gb: zn80d03.s1 Soares_total_fetus_Nb2HF8_{—} | 2.45 | 0.16 | upregulate stage |
| 428227 | AA321649 | Hs.2248 | small inducible cytokine subfamily B (Cy | 9.25 | 0.04 | upregulate stage |
| 428403 | AI393048 | Hs.239894 | leucine rich repeat (in FLII) interactin | 9.94 | 0.06 | upregulate stage |
| 428436 | BE080180 | | gb: RC4-BT0629-120200-011-b10 BT0629 Homo | 1 | 1 | upregulate stage |
| 428450 | NM_014791 | Hs.184339 | KIAA0175 gene product | 4.43 | 0.16 | upregulate stage |
| 428479 | Y00272 | Hs.184572 | cell division cycle 2, G1 to S and G2 to | 9.2 | 0.07 | upregulate stage |
| 428529 | AW262022 | Hs.106278 | Homo sapiens cDNA FLJ12839 fis, clone NT | 1 | 1 | upregulate stage |
| 428576 | AW009330 | Hs.167621 | ESTs | 1 | 0.3 | upregulate stage |
| 428605 | AB037862 | Hs.186756 | KIAA1441 protein | 9.25 | 0.09 | upregulate stage |
| 428664 | AK001666 | Hs.189095 | similar to SALL1 (sal (Drosophila)-like | 3.8 | 0.08 | upregulate stage |
| 428685 | AF131853 | Hs.189527 | Homo sapiens clone 25016 mRNA sequence | 1 | 1 | upregulate stage |
| 428716 | AL122118 | Hs.190614 | Homo sapiens mRNA; cDNA DKFZp434O1221 (f | 1 | 0.65 | upregulate stage |
| 428783 | AW070204 | Hs.178176 | ESTs | 1.6 | 0.23 | upregulate stage |
| 428788 | AF082283 | Hs.193516 | B-cell CLL/lymphoma 10 | 9.6 | 0.08 | upregulate stage |
| 428829 | R14050 | Hs.194051 | Homo sapiens mRNA; cDNA DKFZp566B213 (fr | 5.45 | 0.11 | upregulate stage |
| 428839 | AI767756 | Hs.82302 | ESTs | 10 | 0.06 | upregulate stage |
| 428881 | AI298368 | Hs.98918 | ESTs | 1.4 | 0.18 | upregulate stage |
| 428954 | AF100781 | Hs.194678 | WNT1 inducible signaling pathway protein | 1 | 1 | upregulate stage |
| 428988 | AA442900 | Hs.27947 | ESTs | 3.05 | 0.13 | upregulate stage |
| 429042 | AW015489 | Hs.235920 | ESTs | 1 | 0.56 | upregulate stage |
| 429057 | AF156557 | Hs.194816 | stomatin-like protein 1 | 0.95 | 0.93 | upregulate stage |
| 429066 | AA868555 | Hs.178222 | ESTs | 6 | 0.11 | upregulate stage |
| 429072 | AI376228 | Hs.108043 | Friend leukemia virus integration 1 | 1 | 1 | upregulate stage |
| 429083 | Y09397 | Hs.227817 | BCL2-related protein A1 | 11.12 | 0.03 | upregulate stage |
| 429091 | AA935658 | Hs.187939 | ESTs | 8.9 | 0.08 | upregulate stage |
| 429115 | AA446728 | Hs.289020 | Homo sapiens cDNA FLJ14098 fis, clone MA | 4.1 | 0.17 | upregulate stage |
| 429127 | AA749382 | Hs.107233 | ESTs | 1 | 0.23 | upregulate stage |
| 429135 | AA446966 | Hs.99090 | ESTs, Moderately similar to similar to K | 1 | 1 | upregulate stage |
| 429170 | NM_001394 | Hs.2359 | dual specificity phosphatase 4 | 8.6 | 0.08 | upregulate stage |
| 429174 | BE559598 | Hs.197803 | KIAA0160 protein | 8.4 | 0.06 | upregulate stage |
| 429236 | AA448407 | | gb: zw68d11.s1 Soares_testis_NHT Homo sap | 1 | 0.36 | upregulate stage |
| 429268 | AA205386 | Hs.198481 | RAR-related orphan receptor B | 2.9 | 0.16 | upregulate stage |
| 429300 | AB011108 | Hs.198891 | serine/threonine-protein kinase PRP4 hom | 4.25 | 0.15 | upregulate stage |
| 429334 | D63078 | Hs.186180 | Homo sapiens cDNA: FLJ23038 fis, clone L | 2.95 | 0.11 | upregulate stage |
| 429344 | R94038 | Hs.199538 | inhibin, beta C | 2.91 | 0.28 | upregulate stage |
| 429359 | W00482 | Hs.2399 | matris metalloproteinase 14 (membrane-in | 1.19 | 0.68 | upregulate stage |
| 429376 | AI867889 | Hs.43227 | ESTs | 1 | 1 | upregulate stage |
| 429412 | NM_006235 | Hs.2407 | POU domain, class 2, associating factor | 8.15 | 0.07 | upregulate stage |
| 429450 | AA824451 | Hs.94292 | Homo sapiens cDNA: FLJ23311 fis, clone H | 3.3 | 0.17 | upregulate stage |
| 429472 | AW452421 | Hs.15652 | ESTs | 1 | 1 | upregulate stage |
| 429482 | AF076974 | Hs.203952 | transformation/transcripton domain-asso | 1.52 | 0.59 | upregulate stage |
| 429486 | AF155827 | Hs.203963 | hypothetical protein FLJ10339 | 2.9 | 0.15 | upregulate stage |
| 429572 | AW295375 | Hs.39474 | ESTs | 1 | 0.95 | upregulate stage |
| 429584 | AI817785 | Hs.183037 | protein kinase, cAMP-dependent, regulato | 6.55 | 0.1 | upregulate stage |
| 429590 | AI219490 | Hs.44445 | ESTs, Weakly similar to Kelch motif cont | 1 | 1 | upregulate stage |
| 429597 | NM_003816 | Hs.2442 | a disintegrin and metalloproteinase doma | 5.6 | 0.14 | upregulate stage |
| 429601 | AI804293 | Hs.119406 | ESTs, Weakly similar to AF143946 1 trans | 1.36 | 0.58 | upregulate stage |
| 429602 | AA521463 | Hs.183424 | ESTs | 1 | 0.34 | upregulate stage |
| 429617 | X89984 | Hs.211563 | B-cell CLL/lymphoma 7A | 10.8 | 0.07 | upregulate stage |
| 429629 | BE501732 | Hs.30622 | Homo sapiens cDNA FLJ13010 fis, clone NT | 3.4 | 0.12 | upregulate stage |
| 429631 | AA455612 | Hs.136710 | EST | 1 | 1 | upregulate stage |
| 429644 | AA455892 | Hs.156379 | ESTs | 3.4 | 0.15 | upregulate stage |
| 429653 | NM_005955 | Hs.211581 | metal-regulatory transcription factor 1 | 4.45 | 0.17 | upregulate stage |
| 429664 | L20433 | Hs.211588 | POU domain, class 4, transcription facto | 1.17 | 0.74 | upregulate stage |
| 429673 | AA884407 | Hs.211595 | protein tyrosine phosphatase, non-recept | 3.19 | 0.22 | upregulate stage |
| 429699 | AI383469 | Hs.159300 | ESTs | 4.4 | 0.1 | upregulate stage |
| 429782 | NM_005754 | Hs.220689 | Ras-GTPase-activating protein SH3-domain | 4.25 | 0.13 | upregulate stage |
| 429813 | AW139678 | Hs.180791 | ESTs | 1 | 0.95 | upregulate stage |
| 429828 | AB019494 | Hs.225767 | IDN3 protein | 4.2 | 0.14 | upregulate stage |
| 429838 | AW904907 | Hs.108241 | ESTs, Weakly similar to The KIAA0191 gen | 3.25 | 0.14 | upregulate stage |
| 429859 | NM_007050 | Hs.225952 | protein tyrosine phosphatase, receptor t | 1 | 0.36 | upregulate stage |
| 429913 | AA460608 | Hs.99552 | ESTs | 1.35 | 0.2 | upregulate stage |
| 429917 | H80572 | | gb: yu76c02.r1 Soaren fetal liver spleen | 4.8 | 0.13 | upregulate stage |
| 429921 | AA526911 | Hs.102756 | ESTs | 1 | 0.63 | upregulate stage |
| 429950 | AW081608 | Hs.105053 | ESTs | 3.7 | 0.13 | upregulate stage |
| 429971 | AF079550 | Hs.227098 | glial cells missing (Drosophila) homolog | 1 | 0.83 | upregulate stage |
| 429979 | AA463338 | | gb: zx97a10.r1 Soares_NhHMPu_S1 Homo sapi | 1 | 0.32 | upregulate stage |
| 429982 | AW449534 | Hs.99607 | Homo sapiens cDNA FLJ13841 fis, clone TH | 9.45 | 0.08 | upregulate stage |
| 429986 | AF092047 | Hs.227277 | sine oculis homeobox (Drosophila) homolo | 0.78 | 0.58 | upregulate stage |
| 430020 | AI539029 | Hs.99607 | Homo sapiens cDNA FLJ13841 fis, clone TH | 1 | 0.36 | upregulate stage |
| 430021 | AA463913 | Hs.221160 | ESTs | 1 | 0.56 | upregulate stage |
| 430049 | AW277085 | Hs.99619 | ESTs | 3.55 | 0.17 | upregulate stage |
| 430060 | NM_002941 | Hs.301198 | roundabout (axon guidance receptor, Dros | 1 | 0.59 | upregulate stage |
| 430076 | AA465115 | | gb: aa32c11.r1 NCI_CGAP_GCB1 Homo sapiens | 5.4 | 0.12 | upregulate stage |
| 430134 | BE380149 | Hs.105223 | ESTs, Weakly similar to contains similar | 3.6 | 0.13 | upregulate stage |
| 430184 | AB013802 | Hs.234790 | contactin 5 | 1 | 1 | upregulate stage |
| 430195 | AW969308 | Hs.188594 | ESTs | 9.15 | 0.1 | upregulate stage |
| 430279 | R85974 | Hs.16279 | ESTs | 1.2 | 0.52 | upregulate stage |
| 430287 | AW182459 | Hs.125759 | ESTs, Weakly similar to tumor suppressor | 4.05 | 0.15 | upregulate stage |
| 430291 | AV660345 | Hs.238126 | CGI-49 protein | 7.2 | 0.08 | upregulate stage |
| 430299 | W28673 | Hs.106747 | serine carboxypeptidase 1 precursor prot | 11.9 | 0.06 | upregulate stage |
| 430350 | BE169639 | | gb: PM1-HT0527-280200-005-a05 HT0527 Homo | 7.1 | 0.09 | upregulate stage |
| 430387 | AW372884 | Hs.240770 | nuclear cap binding protein subunit 2, 2 | 5.9 | 0.11 | upregulate stage |
| 430486 | BE062109 | Hs.241551 | chloride channel, calcium activated, fam | 2.56 | 0.2 | upregulate stage |
| 430488 | D19589 | Hs.4220 | ESTs, Moderately similar to tetracycline | 10.5 | 0.08 | upregulate stage |
| 430512 | AF182294 | Hs.241578 | U6 snRNA-associated Sm-like protein LSm8 | 11.2 | 0.06 | upregulate stage |
| 430519 | AF129534 | Hs.49210 | F-box only protein 4 | 5.35 | 0.11 | upregulate stage |
| 430550 | AK000062 | Hs.243756 | hypothetical protein FLJ20055 | 1 | 1 | upregulate stage |
| 430561 | BE065227 | | gb: RC1-BT0314-310300-015-b06 BT0314 Homo | 1 | 1 | upregulate stage |
| 430563 | AA481269 | Hs.178381 | ESTs | 1 | 0.45 | upregulate stage |
| 430598 | AK001764 | Hs.247112 | hypothetical protein FLJ10902 | 4.75 | 0.14 | upregulate stage |
| 430630 | AW269920 | Hs.2621 | cystatin A (stefin A) | 2.52 | 0.25 | upregulate stage |
| 430634 | AI860651 | Hs.26685 | ESTs | 1.24 | 0.61 | upregulate stage |
| 430637 | BE160081 | Hs.256290 | S100 calciam-binding protein A11 (calgiz | 1.79 | 0.47 | upregulate stage |
| 430640 | AA482636 | | gb: zv29c06.r1 Soares ovary tumor NbHOT H | 9.15 | 0.08 | upregulate stage |
| 430665 | BE350122 | Hs.157367 | ESTs | 9.4 | 0.08 | upregulate stage |
| 430726 | AL031224 | Hs.247850 | Human DNA sequence from clone 336H9 on c | 1 | 0.27 | upregulate stage |
| 430733 | AW975920 | Hs.283361 | ESTs | 3.5 | 0.13 | upregulate stage |
| 430781 | AW088127 | Hs.278536 | ESTs | 1 | 1 | upregulate stage |
| 430791 | AA486293 | Hs.272068 | ESTs, Moderately similar to alternativel | 1.61 | 0.42 | upregulate stage |
| 430817 | AA487242 | Hs.185105 | ESTs | 1 | 1 | upregulate stage |
| 430888 | BE155293 | Hs.76064 | ribosomal protein L27a | 3.05 | 0.17 | upregulate stage |
| 430918 | NM_000843 | Hs.248131 | glutamate receptor, metabotropic 6 | 9.05 | 0.09 | upregulate stage |
| 430926 | L05597 | Hs.248136 | 5-hydroxytryptamine (serotonin) receptor | 1 | 0.91 | upregulate stage |
| 430994 | AA490346 | Hs.40530 | ESTs | 1.03 | 0.89 | upregulate stage |
| 431009 | BE149762 | Hs.248213 | gap junction protein, beta 6 (connexin 3 | 24.8 | 0.03 | upregulate stage |
| 431023 | AI283133 | Hs.178925 | ESTs | 2.55 | 0.15 | upregulate stage |
| 431030 | AA830525 | Hs.291988 | ESTs | 1 | 0.47 | upregulate stage |
| 431041 | AA490967 | Hs.105276 | ESTs | 1 | 0.36 | upregulate stage |
| 431070 | AW408164 | Hs.249184 | transcription factor 19 (SC1) | 1.65 | 0.45 | upregulate stage |
| 431082 | AA491600 | Hs.161942 | ESTs | 9.85 | 0.06 | upregulate stage |
| 431089 | BE041395 | Hs.283676 | ESTs, Weakly similar to unknown protein | 43.15 | 0.01 | upregulate stage |
| 431146 | Z83850 | Hs.250649 | Human DNA sequence from PAC 82J11 and co | 1 | 0.4 | upregulate stage |
| 431173 | AW971198 | Hs.294068 | ESTs | 6.3 | 0.12 | upregulate stage |
| 431245 | AA496933 | Hs.191687 | ESTs | 1 | 1 | upregulate stage |
| 431253 | R06428 | Hs.226351 | ESTs | 1 | 0.8 | upregulate stage |
| 431267 | AW969661 | Hs.124047 | ESTs | 1 | 0.31 | upregulate stage |
| 431287 | BE044989 | Hs.274901 | ESTs | 1 | 1 | upregulate stage |
| 431322 | AW970622 | | gb: EST382704 MAGE resequences, MAGK Homo | 10.8 | 0.06 | upregulate stage |
| 431332 | AA503297 | Hs.117108 | ESTs | 6.55 | 0.1 | upregulate stage |
| 431343 | AW970603 | Hs.300941 | Homo sapiens cDNA FLJ11661 fis, clone HE | 5.65 | 0.09 | upregulate stage |
| 431346 | AA371059 | Hs.251636 | ubiquitin specific protease 3 | 1.68 | 0.52 | upregulate stage |
| 431347 | AI133461 | Hs.251664 | insulin-like growth factor 2 (somatomedi | 1.12 | 0.47 | upregulate stage |
| 431381 | AA577114 | Hs.105727 | ESTs | 1 | 0.36 | upregulate stage |
| 431448 | AL137517 | Hs.288381 | hypothetical protein DKFZp564O1278 | 3.72 | 0.13 | upregulate stage |
| 431494 | AA991355 | Hs.129808 | ESTs | 2.75 | 0.18 | upregulate stage |
| 431510 | AA580082 | Hs.112264 | ESTs | 3.75 | 0.13 | upregulate stage |
| 431560 | BE244135 | Hs.260238 | hypothetical protein FLJ10842 | 9.1 | 0.08 | upregulate stage |
| 431571 | AW500486 | Hs.180610 | splicing factor proline/glutamine rich ( | 7.5 | 0.11 | upregulate stage |
| 431596 | T34708 | Hs.272927 | Sec23 (S. cerivisiae) homolog A | 8.2 | 0.08 | upregulate stage |
| 431610 | AK000972 | Hs.264363 | hypothetical protein FLJ10110 | 6.4 | 0.1 | upregulate stage |
| 431613 | AA018515 | Hs.264482 | Apg12 (autophagy 12, S. cerivisiae)-like | 5.8 | 0.11 | upregulate stage |
| 431630 | NM_002204 | Hs.265829 | integrin, alpha 3 (antigen CD49C, alpha | 1.3 | 0.59 | upregulate stage |
| 431663 | NM_016569 | Hs.267182 | TBX3-iso protein | 1.6 | 0.52 | upregulate stage |
| 431670 | AW971287 | | gb: EST383376 MAGE resequences, MAGL Homo | 1 | 1 | upregulate stage |
| 431689 | AA305688 | Hs.267695 | UDP-Gal: betaGlcNAc beta 1,3-galactosyltr | 9.1 | 0.05 | upregulate stage |
| 431691 | AI208511 | Hs.292510 | ESTs | 4.15 | 0.12 | upregulate stage |
| 431692 | AL021331 | Hs.267749 | unc93 (C. elegans) homolog A | 4.2 | 0.13 | upregulate stage |
| 431694 | AW970112 | Hs.292697 | ESTs | 1 | 0.83 | upregulate stage |
| 431726 | NM_015361 | Hs.268053 | KIAA0029 protein | 10.1 | 0.07 | upregulate stage |
| 431736 | AI912234 | Hs.151245 | ESTs | 9.9 | 0.08 | upregulate stage |
| 431753 | X76029 | Hs.2841 | neuromedin U | 1 | 0.23 | upregulate stage |
| 431781 | AA515474 | Hs.99908 | nuclear receptor coactivator 4 | 1 | 0.36 | upregulate stage |
| 431810 | X67155 | Hs.270845 | kinesin-like 5 (mitotic kinesin-like pro | 1 | 0.65 | upregulate stage |
| 431814 | BE256242 | Hs.270847 | delta-tubulin | 3.35 | 0.18 | upregulate stage |
| 431817 | X65233 | Hs.271079 | zinc finger protein 80 (pT17) | 1 | 1 | upregulate stage |
| 431828 | AA572994 | | gb: nm33f12.s1 NCI_CGAP_Lip2 Homo sapiens | 4 | 0.12 | upregulate stage |
| 431880 | AI700238 | Hs.187486 | ESTs | 1 | 1 | upregulate stage |
| 431890 | X17033 | Hs.271986 | integrin, alpha 2 (CD49B, alpha 2 subuni | 2.89 | 0.27 | upregulate stage |
| 431941 | AK000106 | Hs.272227 | Homo sapiens cDNA FLJ20099 fis, clone CO | 1 | 0.18 | upregulate stage |
| 431951 | AI086335 | Hs.136470 | ESTs | 6.4 | 0.11 | upregulate stage |
| 431958 | X63629 | Hs.2877 | cadherin 3, type 1, P-cadherin (placenta | 9.09 | 0.07 | upregulate stage |
| 431989 | AW972870 | Hs.291069 | ESTs | 1 | 0.23 | upregulate stage |
| 431992 | NM_002742 | Hs.2891 | protein kinase C, mu | 3.9 | 0.15 | upregulate stage |
| 432015 | AL157504 | Hs.159115 | ESTs | 6.05 | 0.09 | upregulate stage |
| 432023 | AW273128 | Hs.214188 | ESTs | 0.99 | 0.86 | upregulate stage |
| 432028 | AJ272208 | Hs.272354 | interleukin 1 receptor accessory protein | 1 | 0.48 | upregulate stage |
| 432039 | AF220217 | Hs.272374 | Homo sapiens rsec15-like protein mRNA, p | 1 | 0.24 | upregulate stage |
| 432065 | AA401039 | Hs.2903 | protein phosphatase 4 (formerly X), cata | 1.38 | 0.64 | upregulate stage |
| 432069 | AW975868 | Hs.294100 | ESTs | 4.25 | 0.15 | upregulate stage |
| 432072 | N62937 | Hs.269109 | ESTs | 5.9 | 0.09 | upregulate stage |
| 432093 | H28383 | | gb: yl52c03.r1 Soares breast 3NbHBst Homo | 7.9 | 0.08 | upregulate stage |
| 432136 | AA157632 | Hs.272630 | vacuolar proton pump delta polypeptide | 1 | 0.28 | upregulate stage |
| 432162 | AA584062 | Hs.272798 | hypothetical protein FLJ20413 | 2.5 | 0.25 | upregulate stage |
| 432169 | Y00971 | Hs.2910 | phosphoribosyl pyrophosphate synthetase | 6.1 | 0.11 | upregulate stage |
| 432215 | AU076609 | Hs.2934 | ribonucleotide reductase M1 polypeptide | 2.44 | 0.29 | upregulate stage |
| 432222 | AI204995 | | gb: an03c03.x1 Stratagene schizo brain S1 | 15 | 0.02 | upregulate stage |
| 432235 | AA531129 | Hs.190297 | ESTs | 9.57 | 0.06 | upregulate stage |
| 432237 | AK001926 | Hs.274132 | hypothetical protein FLJ11064 | 1 | 0.44 | upregulate stage |
| 432239 | X81334 | Hs.2936 | matrix metalloproteinase 13 (collagenase | 4.3 | 0.1 | upregulate stage |
| 432281 | AK001239 | Hs.274263 | hypothetical protein FLJ10377 | 3.95 | 0.15 | upregulate stage |
| 432338 | AA534197 | Hs.272693 | ESTs | 1 | 1 | upregulate stage |
| 432374 | W68815 | Hs.301885 | Homo sapiens cDNA FLJ11346 fis, clone PL | 1.97 | 0.4 | upregulate stage |
| 432375 | BE536069 | Hs.2962 | S100 calcium-binding protein P | 1.16 | 0.58 | upregulate stage |
| 432407 | AA221036 | Hs.285026 | HERV-H LTR-associating 1 | 3.75 | 0.16 | upregulate stage |
| 432410 | X68561 | Hs.2982 | Sp4 transcription factor | 1 | 1 | upregulate stage |
| 432415 | T16971 | Hs.289014 | ESTs | 7.3 | 0.07 | upregulate stage |
| 432432 | AA541323 | Hs.115831 | ESTs | 5.35 | 0.13 | upregulate stage |
| 432435 | BE218886 | Hs.282070 | ESTs | 5.35 | 0.1 | upregulate stage |
| 432441 | AW292425 | Hs.163484 | ESTs | 19.4 | 0.04 | upregulate stage |
| 432518 | AI675836 | Hs.94319 | ESTs | 1 | 0.59 | upregulate stage |
| 432580 | X82018 | Hs.3053 | zinc finger protein with interaction dom | 9.15 | 0.08 | upregulate stage |
| 432606 | NM_002104 | Hs.3066 | granzyme K (serine protease, granzyme 3: | 10.15 | 0.05 | upregulate stage |
| 432614 | AA557153 | Hs.185853 | ESTs | 1 | 0.33 | upregulate stage |
| 432642 | BE297635 | Hs.3069 | heat shock 70 kD protein 98 (mortalin-2) | 10.5 | 0.07 | upregulate stage |
| 432661 | AW973823 | Hs.283526 | ESTs | 1 | 1 | upregulate stage |
| 432666 | AW204069 | Hs.129250 | ESTs, Weakly similar to unnamed protein | 1 | 0.16 | upregulate stage |
| 432669 | AL043482 | Hs.267115 | ESTs | 4.15 | 0.12 | upregulate stage |
| 432673 | AB028859 | Hs.278605 | ER-associated DNAJ; ER-associated Hsp40 | 10.24 | 0.06 | upregulate stage |
| 432678 | AA923424 | Hs.135567 | ESTs | 1 | 0.69 | upregulate stage |
| 432690 | AF181490 | Hs.278627 | prenylcysteine lyase | 4.55 | 0.12 | upregulate stage |
| 432724 | X98266 | | gb: H. sapiens mRNA for ligase like protei | 1 | 1 | upregulate stage |
| 432758 | NM_014091 | Hs.278920 | PRO1510 protein | 1 | 1 | upregulate stage |
| 432773 | NM_014124 | Hs.278935 | PRO0255 protein | 1 | 1 | upregulate stage |
| 432789 | D26361 | Hs.3104 | KIAA0042 gene product | 3.46 | 0.22 | upregulate stage |
| 432829 | W60377 | Hs.57772 | ESTs | 1.33 | 0.43 | upregulate stage |
| 432840 | AK001403 | Hs.279521 | hypothetical protein FLJ20530 | 4.5 | 0.14 | upregulate stage |
| 432900 | BE178025 | Hs.7942 | hypothetical protein FLJ20080 | 1 | 0.3 | upregulate stage |
| 432917 | NM_014125 | Hs.279812 | PRO0327 protein | 6.33 | 0.12 | upregulate stage |
| 432935 | AW270239 | Hs.213709 | ESTs | 3.85 | 0.11 | upregulate stage |
| 432963 | AA572859 | Hs.225791 | ESTs | 1 | 0.19 | upregulate stage |
| 433001 | AF217513 | Hs.279905 | clone HQ0310 PRO0310p1 | 29.9 | 0.03 | upregulate stage |
| 433005 | AW939074 | | gb: QV1-DT0069-010200-057-c12 DT0069 Homo | 1 | 0.59 | upregulate stage |
| 433129 | AA577814 | | gb: nn24d03.s1 NCI_CGAP_Gas1 Homo sapiens | 1 | 1 | upregulate stage |
| 433159 | AB035898 | Hs.150587 | kinesin-like protein 2 | 6 | 0.1 | upregulate stage |
| 433201 | AB040896 | Hs.21104 | KIAA1463 protein | 9.2 | 0.09 | upregulate stage |
| 433211 | H11850 | Hs.12808 | MARK | 1.6 | 0.45 | upregulate stage |
| 433218 | AI040372 | Hs.278894 | KIAA1482 protein | 1 | 0.44 | upregulate stage |
| 433222 | AW514472 | Hs.238415 | ESTs, Moderately similar to ALU8_HUMAN A | 5.45 | 0.12 | upregulate stage |
| 433230 | AW136134 | Hs.220277 | ESTs | 7.3 | 0.09 | upregulate stage |
| 433237 | AB040930 | Hs.297021 | Homo sapiens cDNA FLJ13211 fis, clone NT | 1 | 1 | upregulate stage |
| 433365 | AF026944 | Hs.293797 | ESTs | 4.95 | 0.08 | upregulate stage |
| 433371 | T25451 | | gb: PTHI188 HTCDL1 Homo sapiens cDNA 5′/3 | 4.75 | 0.12 | upregulate stage |
| 433394 | AI907753 | Hs.93810 | cerebral cavernous malformations 1 | 4.5 | 0.11 | upregulate stage |
| 433424 | R68252 | Hs.163566 | ESTs | 1 | 1 | upregulate stage |
| 433440 | AF052127 | | gb: Homo sapiens clone 23850 mRNA sequenc | 1 | 1 | upregulate stage |
| 433452 | AW296906 | Hs.142869 | ESTs | 9.82 | 0.08 | upregulate stage |
| 433456 | AA593447 | Hs.124296 | ESTs | 9.45 | 0.08 | upregulate stage |
| 433467 | AI420457 | Hs.50955 | ESTs | 1.11 | 0.74 | upregulate stage |
| 433479 | AW511459 | Hs.249972 | ESTs | 3.35 | 0.13 | upregulate stage |
| 433484 | BE264397 | Hs.148674 | ESTs | 1 | 0.27 | upregulate stage |
| 433515 | AA595800 | Hs.190246 | ESTs | 3.05 | 0.14 | upregulate stage |
| 433602 | AI769948 | Hs.24906 | ESTs | 1 | 1 | upregulate stage |
| 433613 | AA836126 | Hs.5669 | ESTs | 2.8 | 0.12 | upregulate stage |
| 433625 | AW955674 | Hs.161762 | ESTs | 1 | 0.53 | upregulate stage |
| 433658 | L03678 | Hs.156110 | immunoglobulin kappa constant | 9.65 | 0.05 | upregulate stage |
| 433672 | BE281165 | Hs.288038 | TLS-associated serine-arginine protein 1 | 7.9 | 0.08 | upregulate stage |
| 433730 | AK002135 | Hs.3542 | hypothetical protein FLJ11273 | 6.7 | 0.11 | upregulate stage |
| 433735 | AA608955 | Hs.109653 | ESTs | 8.95 | 0.08 | upregulate stage |
| 433895 | AI287912 | Hs.3628 | mitogen-activated protein kinase kinase | 4.43 | 0.16 | upregulate stage |
| 433904 | AI399956 | Hs.208956 | ESTs | 5.5 | 0.12 | upregulate stage |
| 433929 | AI375499 | Hs.27379 | ESTs | 7 | 0.09 | upregulate stage |
| 433966 | AF113017 | Hs.284301 | PRO1268 protein | 7.95 | 0.08 | upregulate stage |
| 433967 | AF113018 | Hs.284302 | PRO1621 protein | 2.65 | 0.1 | upregulate stage |
| 434006 | AF113688 | | gb: Homo sapiens clone FLB4630 | 7.85 | 0.08 | upregulate stage |
| 434037 | AF116601 | Hs.283048 | hypothetical protein PRO0128 | 8.81 | 0.09 | upregulate stage |
| 434064 | AL049045 | Hs.180758 | hypothetical protein PRO0082 | 8.35 | 0.09 | upregulate stage |
| 434085 | AF116673 | Hs.250029 | hypothetical protein PRO1925 | 1 | 1 | upregulate stage |
| 434092 | AA625155 | | gb: at70d06.r1 Soares_NhHMPu_S1 Homo sapi | 1 | 1 | upregulate stage |
| 434094 | AA305599 | Hs.238205 | hypothetical protein PRO2013 | 11.5 | 0.06 | upregulate stage |
| 434138 | AA625804 | | gb: zu86h01.s1 Soares_testis_NHT Homo sap | 3.55 | 0.11 | upregulate stage |
| 434192 | AW387314 | Hs.34371 | ESTs | 1.65 | 0.22 | upregulate stage |
| 434194 | AF119847 | Hs.283940 | Homo sapiens PRO1550 mRNA, partial cds | 7.85 | 0.09 | upregulate stage |
| 434217 | AW014795 | Hs.23349 | ESTs | 3.8 | 0.13 | upregulate stage |
| 434228 | Z42047 | Hs.283978 | Homo sapiens PRO2751 mRNA, complete cds | 5.95 | 0.11 | upregulate stage |
| 434271 | AA897778 | Hs.201677 | ESTs | 1 | 0.38 | upregulate stage |
| 434280 | BE005398 | | gb: CM1-BN0116-150400-189-h02 BN0116 Homo | 9.45 | 0.07 | upregulate stage |
| 434322 | AI125886 | Hs.152727 | ESTs | 2.65 | 0.18 | upregulate stage |
| 434351 | AW974991 | Hs.191852 | ESTs, Weakly similar to ALU1_HUMAN ALU S | 1 | 0.61 | upregulate stage |
| 434354 | AW974912 | Hs.292783 | ESTs | 1 | 1 | upregulate stage |
| 434398 | AA121098 | Hs.3838 | serum-inducible kinase | 10.7 | 0.08 | upregulate stage |
| 434464 | BE063921 | Hs.295971 | ESTs | 10.15 | 0.07 | upregulate stage |
| 434466 | AB037829 | Hs.3862 | regulator of nonsense transcripts 2; DKF | 7.6 | 0.09 | upregulate stage |
| 434484 | W79839 | Hs.104336 | hypothetical protein | 5.1 | 0.15 | upregulate stage |
| 434513 | AF143888 | Hs.18213 | Homo sapiens clone IMAGE: 121736 mRNA seq | 1 | 1 | upregulate stage |
| 434534 | H90477 | Hs.41407 | ESTs | 1 | 0.18 | upregulate stage |
| 434540 | NM_016045 | Hs.5184 | TH1 drosophila homolog | 12.4 | 0.06 | upregulate stage |
| 434569 | AI311295 | Hs.58609 | ESTs | 1.75 | 0.38 | upregulate stage |
| 434575 | AI133446 | Hs.29964 | ESTs | 9.7 | 0.06 | upregulate stage |
| 434627 | AI221894 | Hs.39311 | ESTs | 1.65 | 0.17 | upregulate stage |
| 434629 | AA789081 | Hs.4029 | glioma-amplified sequence-41 | 9 | 0.07 | upregulate stage |
| 434663 | AA641972 | Hs.130058 | ESTs | 4.55 | 0.15 | upregulate stage |
| 434731 | AA648049 | Hs.121518 | ESTs | 8.5 | 0.1 | upregulate stage |
| 434765 | AA831115 | Hs.190473 | ESTs | 1 | 0.71 | upregulate stage |
| 434773 | AA648962 | Hs.152947 | ESTs | 10.55 | 0.08 | upregulate stage |
| 434792 | AA649253 | Hs.132458 | ESTs | 5.45 | 0.11 | upregulate stage |
| 434828 | D90070 | Hs.96 | phorbol-12-myristate-13-acetate-induced | 1 | 0.34 | upregulate stage |
| 434876 | AF160477 | Hs.245781 | Homo sapiens lg superfamily receptor LNI | 1.4 | 0.57 | upregulate stage |
| 434909 | AI479212 | Hs.17283 | hypothetical protein FLJ10890 | 1 | 0.91 | upregulate stage |
| 434926 | BE543269 | Hs.50252 | Homo sapiens HSPC283 mRNA, partial cds | 4.9 | 0.13 | upregulate stage |
| 434939 | AF161422 | Hs.21590 | Homo sapiens HSPC304 mRNA, partial cds | 1 | 1 | upregulate stage |
| 434963 | AW974957 | Hs.288719 | Homo sapiens cDNA FLJ12142 fis, clone MA | 10.75 | 0.07 | upregulate stage |
| 434970 | AW272262 | Hs.250468 | ESTs | 9.05 | 0.08 | upregulate stage |
| 434980 | AW770553 | Hs.293640 | ESTs | 4.95 | 0.14 | upregulate stage |
| 434997 | AW975155 | Hs.292163 | ESTs | 1 | 0.36 | upregulate stage |
| 435013 | H91923 | Hs.110024 | NADH: ubiquinone oxidoreductase MLRQ subu | 1.16 | 0.71 | upregulate stage |
| 435030 | AI203316 | Hs.148655 | ESTs, Weakly similar to ALU1_HUMAN ALU S | 2.95 | 0.14 | upregulate stage |
| 435047 | AA454985 | Hs.54973 | cadherin-like protein VR20 | 3.31 | 0.2 | upregulate stage |
| 435061 | AI651474 | Hs.163944 | ESTs | 1.4 | 0.2 | upregulate stage |
| 435080 | AI831760 | Hs.155111 | ESTs | 9.05 | 0.08 | upregulate stage |
| 435087 | AW975241 | Hs.23567 | ESTs | 1 | 1 | upregulate stage |
| 435108 | AW975018 | Hs.287440 | Homo sapiens cDNA FLJ11692 fis, clone HE | 1 | 0.2 | upregulate stage |
| 435136 | R27299 | Hs.10172 | ESTs | 8.9 | 0.07 | upregulate stage |
| 435159 | AA668879 | Hs.116649 | ESTs | 1.35 | 0.25 | upregulate stage |
| 435162 | AI911044 | Hs.213893 | ESTs | 1 | 1 | upregulate stage |
| 435166 | AI391470 | Hs.158618 | ESTs | 5.5 | 0.12 | upregulate stage |
| 435212 | AW300100 | Hs.164185 | ESTs | 1 | 1 | upregulate stage |
| 435237 | AI026836 | Hs.114689 | ESTs | 8.75 | 0.1 | upregulate stage |
| 435255 | W87434 | Hs.106015 | ESTs, Moderately similar to ALU1_HUMAN A | 3.4 | 0.14 | upregulate stage |
| 435256 | AF193766 | Hs.13872 | cytokine-like protein C17 | 3.2 | 0.14 | upregulate stage |
| 435257 | AA677026 | Hs.191217 | ESTs | 4.5 | 0.12 | upregulate stage |
| 435298 | AA677696 | Hs.189196 | ESTs | 1 | 1 | upregulate stage |
| 435307 | W90610 | Hs.192003 | ESTs | 1 | 0.87 | upregulate stage |
| 435347 | AW014873 | Hs.116963 | ESTs | 2.45 | 0.14 | upregulate stage |
| 435382 | N54493 | | gb: yv40g05.s1 Soares fetal liver spleen | 1 | 0.56 | upregulate stage |
| 435408 | H07897 | Hs.4302 | ESTs | 0.84 | 0.97 | upregulate stage |
| 435491 | T98543 | Hs.191900 | ESTs | 1 | 0.3 | upregulate stage |
| 435525 | AI831297 | Hs.123310 | ESTs | 3.25 | 0.15 | upregulate stage |
| 435597 | AW305188 | Hs.163027 | ESTs | 1 | 0.57 | upregulate stage |
| 435637 | AF220051 | Hs.110853 | uncharacterized hematopoietic stem/proge | 8.76 | 0.09 | upregulate stage |
| 435647 | AI653240 | Hs.49823 | ESTs | 3.19 | 0.25 | upregulate stage |
| 435738 | AA699633 | Hs.269543 | ESTs | 2.9 | 0.16 | upregulate stage |
| 435762 | AW043836 | Hs.212460 | ESTs | 1 | 1 | upregulate stage |
| 435809 | H90213 | Hs.191330 | ESTs | 1.25 | 0.23 | upregulate stage |
| 435826 | AI554089 | Hs.117880 | ESTs | 6.55 | 0.08 | upregulate stage |
| 435854 | AJ278120 | Hs.4996 | DKFZP564D166 protein | 2.76 | 0.29 | upregulate stage |
| 435979 | W03698 | Hs.83513 | ESTs, Weakly similar to ALU1_HUMAN ALU S | 1 | 0.25 | upregulate stage |
| 435981 | H74319 | Hs.188620 | ESTs | 6.35 | 0.11 | upregulate stage |
| 435990 | AI015862 | Hs.131793 | ESTs | 4.25 | 0.08 | upregulate stage |
| 435999 | AA703271 | Hs.269903 | ESTs, Moderately similar to ALU1_HUMAN A | 1 | 1 | upregulate stage |
| 436016 | AA806465 | Hs.121536 | ESTs | 1.45 | 0.23 | upregulate stage |
| 436023 | T81819 | | gb: yd95f05.s1 Soares fetal liver spleen | 9.15 | 0.07 | upregulate stage |
| 436052 | AI021983 | Hs.271432 | ESTs | 1 | 0.23 | upregulate stage |
| 436115 | AW512033 | Hs.102004 | ESTs | 1.9 | 0.21 | upregulate stage |
| 436118 | AI221173 | Hs.145080 | ESTs | 1 | 1 | upregulate stage |
| 436120 | AI248193 | Hs.119860 | ESTs | 9.61 | 0.08 | upregulate stage |
| 436149 | AI754308 | Hs.159452 | ESTs | 2.4 | 0.19 | upregulate stage |
| 436156 | AA705466 | Hs.119900 | ESTs | 1 | 0.26 | upregulate stage |
| 436170 | AW450381 | Hs.14529 | ESTs | 1 | 0.91 | upregulate stage |
| 436202 | AA706315 | Hs.192057 | ESTs | 1 | 1 | upregulate stage |
| 436246 | AW450963 | Hs.119991 | ESTs | 3.85 | 0.11 | upregulate stage |
| 436274 | AA732755 | Hs.120299 | ESTs | 2.85 | 0.13 | upregulate stage |
| 436282 | R91913 | Hs.272104 | ESTs | 3.95 | 0.11 | upregulate stage |
| 436299 | AK000767 | Hs.5111 | hypothetical protein FLJ20729 | 6 | 0.09 | upregulate stage |
| 436300 | AA831601 | Hs.275736 | ESTs | 1 | 1 | upregulate stage |
| 436326 | BE085236 | Hs.181244 | major histocompatibility complex, class | 2.49 | 0.28 | upregulate stage |
| 436360 | AI962796 | Hs.136754 | ESTs | 2.4 | 0.17 | upregulate stage |
| 436363 | AA843926 | Hs.124434 | ESTs | 3.35 | 0.1 | upregulate stage |
| 436383 | BE065178 | | gb: RC1-BT0314-020200-012-h01 BT0314 Homo | 1 | 0.67 | upregulate stage |
| 436396 | AI683487 | Hs.299112 | Homo sapiens cDNA FLJ11441 fis, clone HE | 4 | 0.15 | upregulate stage |
| 436408 | AW274577 | Hs.252954 | ESTs | 9.1 | 0.09 | upregulate stage |
| 436422 | AA716141 | Hs.147027 | ESTs | 1 | 0.26 | upregulate stage |
| 436429 | AA357003 | Hs.17546 | hypothetical protein FLJ23499 | 1 | 1 | upregulate stage |
| 436463 | H06502 | Hs.6656 | ESTs | 5.45 | 0.12 | upregulate stage |
| 436476 | AA326108 | Hs.53631 | ESTs, Weakly similar to enhancer-of-spli | 7.75 | 0.07 | upregulate stage |
| 436507 | AA721209 | Hs.201630 | ESTs | 2.45 | 0.18 | upregulate stage |
| 436518 | AA766433 | Hs.122864 | ESTs | 3.15 | 0.21 | upregulate stage |
| 436522 | AA721381 | Hs.129876 | ESTs | 4.75 | 0.1 | upregulate stage |
| 436578 | AI091435 | Hs.134859 | ESTs | 3.4 | 0.12 | upregulate stage |
| 436670 | AI690021 | Hs.201536 | ESTs | 6.85 | 0.11 | upregulate stage |
| 436740 | AW975133 | | gb: EST387239 MAGE resequences, MAGH Homo | 7.4 | 0.09 | upregulate stage |
| 436764 | AW976004 | Hs.291731 | ESTs | 1 | 1 | upregulate stage |
| 436785 | AA745597 | Hs.291400 | ESTs | 1 | 1 | upregulate stage |
| 436823 | AW749865 | Hs.293645 | ESTs | 4.6 | 0.12 | upregulate stage |
| 436831 | AA830173 | Hs.291918 | ESTs | 1.6 | 0.27 | upregulate stage |
| 436839 | AA767346 | Hs.291614 | ESTs | 1 | 1 | upregulate stage |
| 436844 | AA766458 | Hs.122812 | ESTs | 1.5 | 0.28 | upregulate stage |
| 436853 | BE328074 | Hs.148661 | ESTs | 5.05 | 0.14 | upregulate stage |
| 436860 | H12751 | Hs.5327 | PRO1914 protein | 8.95 | 0.08 | upregulate stage |
| 436925 | AA742327 | Hs.292687 | ESTs | 1 | 1 | upregulate stage |
| 437044 | AL035864 | Hs.69517 | ESTs, Highly similar to differentially e | 1.61 | 0.5 | upregulate stage |
| 437087 | AA745563 | | gb: ny60e04.s1 NCI_CGAP_Pr18 Homo sapiens | 1 | 1 | upregulate stage |
| 437144 | AL049466 | Hs.7859 | ESTs | 1 | 0.31 | upregulate stage |
| 437170 | R49202 | Hs.181694 | ESTs | 8.7 | 0.08 | upregulate stage |
| 437181 | AI306615 | Hs.125343 | ESTs, Weakly similar to KIAA0758 protein | 4.4 | 0.05 | upregulate stage |
| 437207 | T27503 | Hs.15929 | Homo sapiens cDNA FLJ12910 fis, clone NT | 1.4 | 0.22 | upregulate stage |
| 437214 | BE092336 | | gb: IL2-BT0734-240400-072-A12 BT0734 Homo | 5.65 | 0.09 | upregulate stage |
| 437240 | AA747537 | | gb: nx85c05.s1 NCI_CGAP_GCB1 Homo sapiens | 1 | 0.45 | upregulate stage |
| 437257 | AI283085 | Hs.290931 | ESTs, Weakly similar to unknown [S. cerev | 3.8 | 0.14 | upregulate stage |
| 437258 | AL041243 | Hs.174104 | ESTs | 9.72 | 0.08 | upregulate stage |
| 437267 | AW511443 | Hs.258110 | ESTs | 4.25 | 0.12 | upregulate stage |
| 437274 | AA747965 | | gb: nx79a10.s1 NCI_CGAP_Ew1 Homo sapiens | 1 | 0.19 | upregulate stage |
| 437288 | AA748182 | Hs.160377 | ESTs | 1 | 0.61 | upregulate stage |
| 437311 | AA370041 | Hs.9456 | SWI/SNF related, matrix associated, acti | 3.1 | 0.17 | upregulate stage |
| 437324 | AL162077 | | gb: Homo sapiens mRNA; cDNA DKFZp761A219 | 1 | 0.25 | upregulate stage |
| 437330 | AL353944 | Hs.50115 | Homo sapiens mRNA; cDNA DKFZp761J1112 (f | 7.95 | 0.07 | upregulate stage |
| 437356 | BE622396 | Hs.284252 | Homo sapiens mRNA; cDNA DKFZp762O1615 (f | 1 | 1 | upregulate stage |
| 437438 | AL359620 | Hs.14217 | hypothetical protein DKPZp762P2111 | 9.15 | 0.09 | upregulate stage |
| 437471 | AL390169 | | gb: Homo sapiens mRNA; cDNA DKFZp547D064 | 1 | 1 | upregulate stage |
| 437553 | AI829935 | Hs.130497 | ESTs, Weakly similar to MAT8_HUMAN CHLOR | 1.1 | 0.56 | upregulate stage |
| 437567 | AW627990 | Hs.288954 | Homo sapiens cDNA: FLJ21466 fis, clone C | 1 | 1 | upregulate stage |
| 437575 | AW954355 | Hs.36529 | ESTs | 10.25 | 0.06 | upregulate stage |
| 437717 | AA804765 | Hs.132853 | ESTs | 1 | 0.77 | upregulate stage |
| 437722 | AW292947 | Hs.122872 | ESTs | 9.75 | 0.05 | upregulate stage |
| 437752 | AA767376 | Hs.291631 | ESTs | 6.7 | 0.08 | upregulate stage |
| 437770 | AA767881 | Hs.122897 | ESTs | 2.57 | 0.24 | upregulate stage |
| 437798 | AW811767 | | gb: RC2-ST0165-300999-011-g02 ST0165 Homo | 1 | 1 | upregulate stage |
| 437799 | R51083 | Hs.90787 | ESTs | 1 | 1 | upregulate stage |
| 437852 | BE001836 | Hs.256897 | ESTs, Weakly similar to dJ365O12.1 [H. sa | 1.77 | 0.3 | upregulate stage |
| 437886 | BE264111 | Hs.31314 | retinoblastoma-binding protein 7 | 8.8 | 0.08 | upregulate stage |
| 437887 | AA811524 | Hs.29263 | Homo sapiens cDNA FLJ11896 fis, clone HE | 3.8 | 0.17 | upregulate stage |
| 437889 | AA830524 | Hs.124357 | ESTs | 1 | 0.69 | upregulate stage |
| 437937 | AI917222 | Hs.121655 | ESTs | 1 | 0.38 | upregulate stage |
| 437938 | AI950087 | | gb: wq05c02.x1 NCI_CGAP_Kid12 Homo sapien | 1.37 | 0.52 | upregulate stage |
| 437983 | AI303023 | Hs.30211 | hypothetical protein FLJ22313 | 8.82 | 0.08 | upregulate stage |
| 438011 | BE466173 | Hs.145696 | splicing factor (CC1.3) | 9 | 0.09 | upregulate stage |
| 438032 | BE045624 | Hs.152992 | ESTs | 5.65 | 0.13 | upregulate stage |
| 438069 | N80701 | Hs.33790 | ESTs | 3.25 | 0.13 | upregulate stage |
| 438077 | AA777330 | Hs.50429 | ESTs | 1 | 1 | upregulate stage |
| 438081 | H49546 | Hs.298964 | ESTs | 3.75 | 0.11 | upregulate stage |
| 438102 | AA777793 | Hs.137580 | xylulokinase (H. inflsunzae) homolog | 1 | 1 | upregulate stage |
| 438112 | W85729 | Hs.194279 | ESTs | 1 | 0.33 | upregulate stage |
| 438113 | AI467908 | Hs.8882 | ESTs | 1.21 | 0.55 | upregulate stage |
| 438119 | AW963217 | Hs.203961 | ESTs, Moderately similar to AF116721 89 | 11.75 | 0.07 | upregulate stage |
| 438144 | AA778894 | Hs.118364 | ESTs | 1 | 1 | upregulate stage |
| 438153 | AI268632 | Hs.146159 | ESTs | 1 | 1 | upregulate stage |
| 438171 | AW976507 | Hs.293515 | ESTs | 2.82 | 0.24 | upregulate stage |
| 438271 | L21934 | Hs.14553 | sterol O-acyltransferase (acyl-Coenzyme | 1 | 1 | upregulate stage |
| 438290 | AA843719 | Hs.122341 | ESTs | 3.9 | 0.13 | upregulate stage |
| 438321 | AA576635 | Hs.6153 | CGI-48 protein | 9.4 | 0.08 | upregulate stage |
| 438325 | AA804258 | Hs.123229 | ESTs | 4.65 | 0.11 | upregulate stage |
| 438334 | AA806992 | Hs.291686 | ESTs | 1 | 1 | upregulate stage |
| 438366 | AA805760 | | gb: ns43f01.s1 NCI_CGAP_GCB1 Homo sapiens | 1 | 0.34 | upregulate stage |
| 438370 | AA843242 | Hs.48523 | ESTs | 4 | 0.14 | upregulate stage |
| 438374 | AA321866 | Hs.6193 | Homo sapiens mRNA; cDNA DKFZp434C717 (f | 1 | 0.84 | upregulate stage |
| 438377 | AA806070 | Hs.291716 | ESTs | 1 | 0.24 | upregulate stage |
| 438378 | AW970529 | Hs.86434 | Homo sapiens cDNA: FLJ21816 fis, clone H | 6.65 | 0.11 | upregulate stage |
| 438401 | AL046321 | Hs.197484 | ESTs | 1 | 1 | upregulate stage |
| 438403 | AA806607 | Hs.292206 | ESTs | 2.75 | 0.14 | upregulate stage |
| 438412 | AA806776 | Hs.130814 | ESTs | 1 | 1 | upregulate stage |
| 438448 | AA807344 | Hs.172932 | Homo sapiens mRNA for partial 3'UTR, seq | 1 | 0.34 | upregulate stage |
| 438451 | AI081972 | Hs.220261 | ESTs | 5.7 | 0.09 | upregulate stage |
| 438473 | H07986 | Hs.136901 | ESTs | 1 | 1 | upregulate stage |
| 438487 | AI684733 | Hs.88820 | HDCMC28P protein | 1 | 0.21 | upregulate stage |
| 438529 | AW007287 | Hs.28538 | Homo sapiens cDNA: FLJ21086 fis, clone C | 1 | 0.36 | upregulate stage |
| 438534 | AW204052 | Hs.123644 | ESTs | 1 | 0.37 | upregulate stage |
| 438693 | AA814369 | Hs.249595 | ESTs | 3.55 | 0.15 | upregulate stage |
| 438698 | AW297855 | Hs.125815 | ESTs | 3.12 | 0.22 | upregulate stage |
| 438728 | AA815202 | Hs.25657 | ESTs | 1 | 0.67 | upregulate stage |
| 438746 | AI885815 | Hs.184727 | ESTs | 1.5 | 0.35 | upregulate stage |
| 438805 | AA826048 | Hs.117887 | ESTs | 9.35 | 0.07 | upregulate stage |
| 438812 | AA826199 | Hs.44287 | ESTs | 1 | 0.57 | upregulate stage |
| 438817 | AI023799 | Hs.163242 | ESTs | 4.2 | 0.08 | upregulate stage |
| 438886 | AA827728 | Hs.128705 | ESTs, Weakly similar to AF149422 2 unkno | 4.05 | 0.12 | upregulate stage |
| 438913 | AI380429 | Hs.172445 | ESTs | 5.4 | 0.11 | upregulate stage |
| 438950 | H23789 | Hs.144530 | ESTs | 1 | 1 | upregulate stage |
| 438961 | H42135 | Hs.101848 | ESTs | 7.85 | 0.08 | upregulate stage |
| 438990 | AF085890 | | gb: Homo sapiens full length insert cDNA | 1 | 0.83 | upregulate stage |
| 439026 | R98978 | Hs.117767 | ESTs | 1 | 0.27 | upregulate stage |
| 439052 | AF085917 | Hs.37921 | ESTs | 1 | 0.22 | upregulate stage |
| 439057 | H59623 | Hs.271561 | ESTs | 1 | 1 | upregulate stage |
| 439176 | AI446444 | Hs.190394 | ESTs | 5.8 | 0.12 | upregulate stage |
| 439179 | AA831250 | Hs.292693 | ESTs | 1 | 1 | upregulate stage |
| 439183 | AW970600 | | gb: EST382681 MAGE resequences, MAGK Homo | 4.5 | 0.13 | upregulate stage |
| 439208 | AK000299 | Hs.180952 | dynactin p62 subunit | 11.9 | 0.06 | upregulate stage |
| 439212 | AF087995 | Hs.134877 | ESTs | 11.4 | 0.07 | upregulate stage |
| 439223 | AW238299 | Hs.23945 | ESTs | 2.79 | 0.26 | upregulate stage |
| 439312 | AA833902 | Hs.270745 | ESTs | 8.9 | 0.08 | upregulate stage |
| 439330 | AF086147 | | gb: Homo sapiens full length insert cDNA | 1 | 0.19 | upregulate stage |
| 439351 | W37688 | Hs.55158 | ESTs, Weakly similar to weak similarity | 1 | 0.31 | upregulate stage |
| 439430 | AF124250 | Hs.6564 | breast cancer anti-estrogen resistance 3 | 3.9 | 0.15 | upregulate stage |
| 439444 | AI277652 | Hs.54578 | ESTs | 11 | 0.07 | upregulate stage |
| 439453 | BE264974 | Hs.6566 | thyroid hormone receptor interactor 13 | 12.21 | 0.05 | upregulate stage |
| 439476 | AA836340 | Hs.165490 | ESTs | 1 | 0.65 | upregulate stage |
| 439492 | AF086310 | Hs.103159 | ESTs | 5.43 | 0.1 | upregulate stage |
| 439527 | AW298119 | Hs.202536 | ESTs | 5.25 | 0.1 | upregulate stage |
| 439550 | H10438 | | gb: ym08d10.s1 Scares infant brain 1NIB H | 3.2 | 0.18 | upregulate stage |
| 439560 | BE565647 | Hs.74899 | hypothetical protein FLJ12820 | 1.81 | 0.32 | upregulate stage |
| 439565 | AF086386 | Hs.145599 | ESTs | 8.96 | 0.07 | upregulate stage |
| 439592 | AF086413 | Hs.58399 | ESTs | 1 | 1 | upregulate stage |
| 439605 | AF086431 | Hs.134805 | ESTs | 9.15 | 0.09 | upregulate stage |
| 439606 | W79123 | Hs.58561 | ESTs, Weakly similar to KI01 HUMAN PROBA | 8.45 | 0.06 | upregulate stage |
| 439750 | AL359053 | Hs.57664 | Homo sapiens mRNA full length insert cDN | 5.43 | 0.14 | upregulate stage |
| 439780 | AL109688 | | gb: Homo sapiens mRNA full length insert | 5 | 0.09 | upregulate stage |
| 439851 | AI149520 | Hs.144453 | ESTs | 2.75 | 0.21 | upregulate stage |
| 439862 | AI571647 | Hs.146170 | hypothetical protein FLJ22969 | 6.3 | 0.11 | upregulate stage |
| 439926 | AW014875 | Hs.137007 | ESTs | 33.5 | 0.02 | upregulate stage |
| 439942 | AW993791 | Hs.94881 | ESTs | 9.9 | 0.08 | upregulate stage |
| 439963 | AW247529 | Hs.6793 | platelet-activating factor acetylhydrola | 5.59 | 0.15 | upregulate stage |
| 439979 | AW600291 | Hs.6823 | hypothetical protein FLJ10430 | 6.95 | 0.07 | upregulate stage |
| 439987 | AA860116 | Hs.223232 | ESTs | 2.45 | 0.17 | upregulate stage |
| 439999 | AA115811 | Hs.6838 | ras homolog gene family, member E | 8.75 | 0.07 | upregulate stage |
| 440006 | AK000517 | Hs.6844 | hypothetical protein FLJ20510 | 1.68 | 0.41 | upregulate stage |
| 440012 | AA861072 | | gb: ak32e05.s1 Soares_testis_NHT Homo sap | 1 | 1 | upregulate stage |
| 440126 | AA975145 | Hs.66194 | ESTs | 1 | 1 | upregulate stage |
| 440194 | R43809 | Hs.22688 | ESTs | 1 | 1 | upregulate stage |
| 440228 | AF125392 | Hs.7089 | insulin induced protein 2 | 1 | 1 | upregulate stage |
| 440249 | AI246590 | Hs.125325 | ESTs | 1.74 | 0.44 | upregulate stage |
| 440284 | AA912032 | Hs.181059 | ESTs | 1 | 1 | upregulate stage |
| 440334 | BE276112 | Hs.7165 | zinc finger protein 259 | 9.45 | 0.09 | upregulate stage |
| 440348 | AW015802 | Hs.47023 | ESTs | 1 | 0.33 | upregulate stage |
| 440351 | AF030933 | Hs.7179 | RAD1 (S. pombe) homolog | 3.75 | 0.16 | upregulate stage |
| 440366 | F08229 | Hs.125273 | ESTs | 3.5 | 0.12 | upregulate stage |
| 440462 | T71629 | Hs.100554 | ESTs | 1.54 | 0.52 | upregulate stage |
| 440527 | AV657117 | Hs.184164 | ESTs | 3.75 | 0.14 | upregulate stage |
| 440613 | AI733034 | Hs.137079 | ESTs | 3.9 | 0.11 | upregulate stage |
| 440705 | AA904244 | Hs.153205 | ESTs | 3.9 | 0.14 | upregulate stage |
| 440856 | AW993377 | Hs.130390 | ESTs | 8.95 | 0.09 | upregulate stage |
| 440899 | AW449445 | Hs.172690 | diacylglycerol kinase, alpha (80 kD) | 2.55 | 0.2 | upregulate stage |
| 440917 | AA909651 | Hs.160025 | ESTs | 1 | 0.17 | upregulate stage |
| 440980 | AL042005 | Hs.1117 | tripeptidyl peptidase II | 8.9 | 0.09 | upregulate stage |
| 440994 | AI160011 | Hs.193341 | ESTs | 1.29 | 0.58 | upregulate stage |
| 441092 | T99289 | Hs.126556 | EST | 4.9 | 0.11 | upregulate stage |
| 441107 | AA917075 | Hs.190520 | ESTs | 5.9 | 0.09 | upregulate stage |
| 441131 | AI733222 | Hs.126632 | ESTs | 9.55 | 0.09 | upregulate stage |
| 441143 | AI027604 | Hs.159650 | ESTs | 3.8 | 0.13 | upregulate stage |
| 441205 | AW137827 | Hs.176904 | ESTs | 4.75 | 0.12 | upregulate stage |
| 441206 | BE552314 | Hs.131823 | ESTs, Weakly similar to TERA HUMAN [H. sa | 1 | 1 | upregulate stage |
| 441264 | AA927170 | Hs.23290 | ESTs | 4.3 | 0.14 | upregulate stage |
| 441318 | AI078234 | Hs.176130 | ESTs | 1.74 | 0.45 | upregulate stage |
| 441334 | AI700529 | Hs.117964 | ESTs | 1 | 1 | upregulate stage |
| 441346 | AA931077 | Hs.186889 | Homo sapiens cDNA FLJ12021 fis, clone HE | 1 | 1 | upregulate stage |
| 441378 | AA931826 | Hs.126846 | ESTs | 4.5 | 0.1 | upregulate stage |
| 441383 | AW294408 | Hs.222068 | ESTs | 1 | 1 | upregulate stage |
| 441421 | AA356792 | Hs.301786 | ESTs | 1 | 0.24 | upregulate stage |
| 441470 | BE503874 | Hs.301986 | ESTs | 0.63 | 0.93 | upregulate stage |
| 441474 | AW274946 | Hs.144476 | ESTs | 1 | 1 | upregulate stage |
| 441484 | AA935481 | Hs.58972 | ESTs | 1 | 0.33 | upregulate stage |
| 441485 | AI792988 | Hs.189133 | ESTs | 4.25 | 0.1 | upregulate stage |
| 441508 | AW015203 | Hs.232237 | ESTs | 1 | 1 | upregulate stage |
| 441562 | AW578981 | Hs.52184 | hypothetical protein FLJ20618 | 4.05 | 0.12 | upregulate stage |
| 441599 | AW473362 | Hs.127221 | ESTs | 1 | 0.29 | upregulate stage |
| 441612 | AI802629 | Hs.113660 | Homo sapiens cDNA FLJ11631 fis, clone HE | 8.75 | 0.08 | upregulate stage |
| 441616 | BE569122 | Hs.74111 | RNA-binding protein (autoantigenic) | 1.14 | 0.71 | upregulate stage |
| 441643 | AI740504 | Hs.205128 | ESTs | 1 | 0.33 | upregulate stage |
| 441677 | AW271702 | Hs.93739 | ESTs | 1 | 0.28 | upregulate stage |
| 441693 | AA384673 | Hs.7943 | RPB5-mediating protein | 1 | 0.43 | upregulate stage |
| 441703 | AW390054 | Hs.192843 | ESTs | 9.85 | 0.08 | upregulate stage |
| 441732 | AW298818 | Hs.127341 | ESTs | 4.6 | 0.14 | upregulate stage |
| 441759 | C16126 | Hs.161377 | ESTs | 4.55 | 0.13 | upregulate stage |
| 441762 | AW592203 | Hs.144769 | ESTs | 1 | 0.83 | upregulate stage |
| 441790 | AW294909 | Hs.132208 | ESTs | 9 | 0.08 | upregulate stage |
| 441794 | AW197794 | Hs.253338 | ESTs | 4.5 | 0.12 | upregulate stage |
| 441799 | AW292276 | Hs.127872 | ESTs | 1 | 0.22 | upregulate stage |
| 441801 | AW242799 | Hs.211874 | ESTs | 8 | 0.06 | upregulate stage |
| 441904 | AI633206 | Hs.128104 | ESTs | 2 | 0.19 | upregulate stage |
| 441955 | AA972327 | Hs.142903 | ESTs | 0.87 | 0.96 | upregulate stage |
| 441989 | AA306207 | Hs.286241 | Homo sapiens cDNA: FLJ22698 fis, clone H | 9.17 | 0.07 | upregulate stage |
| 441990 | T66139 | Hs.113631 | ESTs | 3.55 | 0.12 | upregulate stage |
| 442029 | AW956698 | Hs.14456 | neural precursor cell expressed, develop | 4.65 | 0.14 | upregulate stage |
| 442030 | W67167 | Hs.109080 | ESTs | 1 | 0.35 | upregulate stage |
| 442064 | AI422867 | Hs.88594 | ESTs | 8.8 | 0.08 | upregulate stage |
| 442071 | BE048433 | Hs.276043 | ESTs | 9.15 | 0.09 | upregulate stage |
| 442093 | AA976049 | Hs.128464 | ESTs | 1 | 1 | upregulate stage |
| 442194 | AA984389 | Hs.205088 | ESTs | 1 | 0.83 | upregulate stage |
| 442202 | BE272862 | Hs.106534 | Home sapiens cDNA: FLJ22625 fis, clone H | 9.9 | 0.08 | upregulate stage |
| 442203 | AI921423 | Hs.250146 | ESTs | 1 | 1 | upregulate stage |
| 442214 | AI681733 | Hs.129003 | ESTs | 2.2 | 0.26 | upregulate stage |
| 442216 | AI733468 | Hs.129006 | ESTs | 1 | 1 | upregulate stage |
| 442295 | AI827248 | Hs.224398 | Homo sapiens cDNA FLJ11469 fis, clone HE | 1.49 | 0.31 | upregulate stage |
| 442319 | BE048144 | Hs.177677 | CGI-102 protein | 1 | 0.29 | upregulate stage |
| 442432 | BE093589 | Hs.38178 | Homo sapiens cDNA: FLJ23468 fis, clone H | 22.95 | 0.03 | upregulate stage |
| 442510 | AF150179 | Hs.249890 | ESTs | 1 | 0.63 | upregulate stage |
| 442518 | AF150226 | | gb: AF150226 Human mRNA from cd34 + stem c | 1 | 1 | upregulate stage |
| 442539 | AL119506 | Hs.58220 | Homo sapiens cDNA: FLJ23005 fis, clone L | 1 | 0.23 | upregulate stage |
| 442552 | R20624 | Hs.83572 | son of sevenless (Drosophila) homolog 1 | 9 | 0.08 | upregulate stage |
| 442562 | BE379584 | Hs.34789 | ESTs | 6.55 | 0.1 | upregulate stage |
| 442564 | AI590207 | Hs.188378 | ESTs | 1 | 1 | upregulate stage |
| 442577 | AA292998 | Hs.163900 | ESTs | 1.41 | 0.52 | upregulate stage |
| 442590 | AI002686 | Hs.130313 | ESTs | 1 | 0.36 | upregulate stage |
| 442597 | AI499214 | Hs.130825 | ESTs | 1 | 1 | upregulate stage |
| 442611 | BE077155 | Hs.177537 | ESTs | 4.35 | 0.15 | upregulate stage |
| 442612 | AI005233 | Hs.130631 | ESTs | 1 | 0.28 | upregulate stage |
| 442619 | AA447492 | Hs.20183 | ESTs, Weakly similar to AF164793 1 prote | 7.85 | 0.09 | upregulate stage |
| 442642 | R51853 | Hs.226429 | ESTs | 1 | 1 | upregulate stage |
| 442660 | AW138174 | Hs.130651 | ESTs | 4.1 | 0.09 | upregulate stage |
| 442696 | BE566962 | Hs.7063 | Homo sapiens cDNA: FLJ20913 fis, clone A | 2.65 | 0.16 | upregulate stage |
| 442712 | BE465168 | Hs.131011 | ESTs | 2.51 | 0.23 | upregulate stage |
| 442760 | BE075297 | Hs.10067 | ESTs, Weakly similar to KIAA1205 protein | 8 | 0.1 | upregulate stage |
| 442769 | AW243058 | Hs.131155 | ESTs | 1 | 0.3 | upregulate stage |
| 442785 | AW296625 | Hs.131188 | ESTs | 1 | 0.27 | upregulate stage |
| 442806 | AW294522 | Hs.149991 | ESTs | 9.6 | 0.08 | upregulate stage |
| 442856 | H56735 | Hs.282958 | Homo sapiens cDNA FLJ13611 fis, clone PL | 4.3 | 0.11 | upregulate stage |
| 442861 | AA243837 | Hs.57787 | ESTs | 3.9 | 0.12 | upregulate stage |
| 442875 | BE623003 | Hs.23625 | Homo sapiens clone TCCCTA00142 mRNA sequ | 3.85 | 0.15 | upregulate stage |
| 442879 | AF032922 | Hs.8813 | syntaxin binding protein 3 | 3.95 | 0.14 | upregulate stage |
| 442883 | AW195774 | Hs.253199 | ESTs | 1 | 1 | upregulate stage |
| 442961 | BE614474 | Hs.289074 | Homo sapiens cDNA FLJ13986 fis, clone Y7 | 11.55 | 0.07 | upregulate stage |
| 442966 | AI394036 | Hs.132237 | ESTs, Weakly similar to dual specificity | 2.95 | 0.16 | upregulate stage |
| 442980 | AA857025 | Hs.8878 | kinesin-like 1 | 1 | 0.24 | upregulate stage |
| 442992 | AI914699 | Hs.13297 | ESTs | 6.1 | 0.14 | upregulate stage |
| 442994 | AI026718 | Hs.16954 | ESTs | 8.9 | 0.07 | upregulate stage |
| 443054 | AI745185 | Hs.8939 | yes-associated protein 65 kDa | 5.7 | 0.11 | upregulate stage |
| 443113 | AI040686 | Hs.132908 | ESTs | 3.9 | 0.14 | upregulate stage |
| 443119 | AA312264 | Hs.7980 | ESTs, Moderately similar to ALU4_HUMAN A | 8.73 | 0.08 | upregulate stage |
| 443171 | BE281128 | Hs.9030 | TONDU | 3.18 | 0.22 | upregulate stage |
| 443211 | AI128388 | Hs.143655 | ESTs | 6.55 | 0.08 | upregulate stage |
| 443242 | BE243910 | Hs.9082 | nucleoporin p54 | 11.05 | 0.06 | upregulate stage |
| 443243 | AI452496 | Hs.132056 | ESTs | 8.9 | 0.09 | upregulate stage |
| 443247 | BE614387 | Hs.47378 | ESTs, Moderately similar to hypothetical | 10.95 | 0.05 | upregulate stage |
| 443270 | NM_004272 | Hs.9192 | Homer, neuronal immediate early gene, 1B | 3.75 | 0.2 | upregulate stage |
| 443299 | AI733642 | Hs.133042 | ESTs | 1 | 0.69 | upregulate stage |
| 443362 | AI053464 | Hs.166505 | ESTs | 2.9 | 0.16 | upregulate stage |
| 443383 | AI792453 | Hs.166507 | ESTs | 5 | 0.14 | upregulate stage |
| 443411 | AW134566 | Hs.65320 | ESTs | 1 | 0.59 | upregulate stage |
| 443426 | AF098158 | Hs.9329 | chromosome 20 open reading frame 1 | 2.68 | 0.31 | upregulate stage |
| 443447 | AI094222 | Hs.166572 | ESTs | 1 | 0.38 | upregulate stage |
| 443542 | AI927065 | Hs.146040 | ESTs | 5.65 | 0.13 | upregulate stage |
| 443557 | AV645987 | Hs.145681 | ESTs | 1 | 1 | upregulate stage |
| 443584 | AI807036 | Hs.101619 | ESTs | 1 | 0.36 | upregulate stage |
| 443606 | AI078664 | Hs.199424 | ESTs | 1.15 | 0.33 | upregulate stage |
| 443634 | H73972 | Hs.134460 | ESTs | 3.05 | 0.16 | upregulate stage |
| 443640 | AI872643 | Hs.134218 | ESTs | 3.65 | 0.12 | upregulate stage |
| 443715 | AI583187 | Hs.9700 | cyclin E1 | 5.65 | 0.11 | upregulate stage |
| 443799 | AA150320 | Hs.9800 | protein kinase Njmu-R1 | 1.8 | 0.19 | upregulate stage |
| 443899 | AW842283 | Hs.79933 | cyclin I | 4.65 | 0.13 | upregulate stage |
| 443917 | AW503739 | Hs.72325 | Human DNA sequence from clone RP1-187J11 | 1 | 1 | upregulate stage |
| 443919 | AI091284 | Hs.135224 | ESTs | 8.05 | 0.07 | upregulate stage |
| 443967 | AW294013 | Hs.200942 | ESTs | 5.55 | 0.13 | upregulate stage |
| 443977 | AL120986 | Hs.150627 | ESTs | 4 | 0.14 | upregulate stage |
| 443979 | AV647366 | Hs.282365 | ESTs | 1 | 1 | upregulate stage |
| 444020 | R92962 | Hs.35052 | ESTs | 10.45 | 0.08 | upregulate stage |
| 444105 | AW189097 | Hs.166597 | ESTs | 6.29 | 0.1 | upregulate stage |
| 444129 | AW294292 | Hs.256212 | ESTs | 1 | 0.77 | upregulate stage |
| 444152 | AI125694 | Hs.149305 | Homo sapiens cDNA FLJ14264 fis, clone PL | 1.64 | 0.48 | upregulate stage |
| 444163 | AI126098 | | gb: qc54g07.x1 Soares_placenta_8to9weeks_{—} | 1.12 | 0.81 | upregulate stage |
| 444166 | AV648429 | Hs.282393 | ESTs | 1 | 1 | upregulate stage |
| 444270 | AI138580 | Hs.255220 | EST | 1 | 0.47 | upregulate stage |
| 444271 | AW452569 | Hs.149804 | ESTs | 3.2 | 0.12 | upregulate stage |
| 444282 | AI138955 | | gb: qd79b07.x1 Soares_testis_NHT Homo sap | 1 | 1 | upregulate stage |
| 444333 | AI262567 | Hs.253801 | trinucleotide repeat containing 15 | 1 | 0.77 | upregulate stage |
| 444342 | NM_014398 | Hs.10887 | similar to lysosome-associated membrane | 6.9 | 0.06 | upregulate stage |
| 444378 | R41339 | Hs.12569 | ESTs | 1 | 0.32 | upregulate stage |
| 444381 | BE387335 | Hs.283713 | ESTs, Weakly similar to CA54_HUMAN COLLA | 2.85 | 0.26 | upregulate stage |
| 444431 | AW513324 | Hs.42280 | ESTs | 6.27 | 0.12 | upregulate stage |
| 444437 | AI377961 | Hs.44041 | ESTs | 9.9 | 0.07 | upregulate stage |
| 444444 | AI149332 | Hs.14855 | ESTs | 1.16 | 0.47 | upregulate stage |
| 444525 | AW593778 | Hs.259699 | ESTs | 1 | 0.5 | upregulate stage |
| 444584 | AI168422 | | gb: ok30e11.x1 Soares_NSF_F8_9W_OT_PA_P_S | 3.6 | 0.15 | upregulate stage |
| 444599 | AI174377 | Hs.143796 | ESTs | 1 | 0.44 | upregulate stage |
| 444646 | AI184565 | | gb: qd60b08.x1 Soares_testis_NHT Homo sap | 1 | 1 | upregulate stage |
| 444649 | AW207523 | Hs.197628 | ESTs | 9.35 | 0.06 | upregulate stage |
| 444675 | AI186380 | Hs.244621 | ESTs | 9.88 | 0.08 | upregulate stage |
| 444698 | AI188139 | Hs.147050 | ESTs | 1 | 0.36 | upregulate stage |
| 444743 | AA045648 | Hs.11817 | nudix (nucleoside diphosphate linked moi | 2.1 | 0.15 | upregulate stage |
| 444762 | AI733700 | Hs.143883 | ESTs | 3.9 | 0.14 | upregulate stage |
| 444781 | NM_014400 | Hs.11950 | GPI-anchored metastasis-associated prote | 1.44 | 0.53 | upregulate stage |
| 444783 | AK001468 | Hs.62180 | anillin (Drosophila Scraps homolog), act | 6.65 | 0.07 | upregulate stage |
| 444838 | AV651680 | Hs.208558 | ESTs | 4.84 | 0.14 | upregulate stage |
| 444849 | AI199438 | Hs.148480 | ESTs | 3.05 | 0.17 | upregulate stage |
| 444950 | AI950256 | Hs.224875 | ESTs | 1 | 0.51 | upregulate stage |
| 445027 | AV652692 | Hs.282498 | ESTs | 11.1 | 0.08 | upregulate stage |
| 445091 | AI652154 | Hs.147294 | ESTs | 1 | 1 | upregulate stage |
| 445098 | AL050272 | Hs.12305 | DKFZP566B183 protein | 9.75 | 0.07 | upregulate stage |
| 445101 | T75202 | Hs.12314 | Homo sapiens mRNA; cDNA DKFZp586C1019 (f | 10.9 | 0.07 | upregulate stage |
| 445250 | AI597838 | Hs.175621 | ESTs | 9.8 | 0.08 | upregulate stage |
| 445258 | AI635931 | Hs.147613 | ESTs | 3.05 | 0.13 | upregulate stage |
| 445390 | AI222165 | Hs.144923 | ESTs | 10.6 | 0.06 | upregulate stage |
| 445396 | BE181792 | | gb: QV1-HT0639-070500-193-g06 HT0639 Homo | 1 | 0.29 | upregulate stage |
| 445413 | AA151342 | Hs.12677 | CGI-147 protein | 4.65 | 0.12 | upregulate stage |
| 445436 | AI224105 | Hs.151408 | ESTs | 1.35 | 0.22 | upregulate stage |
| 445444 | AA380876 | Hs.270 | pleckstrin homology, Sec7 and coiled/coi | 10.7 | 0.07 | upregulate stage |
| 445483 | AI307150 | Hs.148845 | ESTs | 1 | 1 | upregulate stage |
| 445496 | AB007860 | Hs.12802 | development and differentiation enhancin | 12.05 | 0.06 | upregulate stage |
| 445525 | BE149866 | Hs.14831 | ESTs | 10.65 | 0.06 | upregulate stage |
| 445527 | W39694 | Hs.83286 | ESTs | 4.85 | 0.1 | upregulate stage |
| 445537 | AJ245671 | Hs.12844 | EGF-like-domain, multiple 6 | 9.7 | 0.06 | upregulate stage |
| 445546 | AW468821 | Hs.156054 | ESTs | 4.2 | 0.13 | upregulate stage |
| 445576 | AI793233 | Hs.145608 | ESTs | 1 | 0.31 | upregulate stage |
| 445623 | AI245366 | Hs.149158 | ESTs | 1 | 1 | upregulate stage |
| 445640 | AW969626 | Hs.31704 | ESTs, Weakly similar to KIAA0227 [H. sapi | 4.7 | 0.13 | upregulate stage |
| 445668 | AI557114 | Hs.181591 | EST | 1 | 0.34 | upregulate stage |
| 445766 | AI623607 | Hs.282977 | Homo sapiens cDNA FLJ13490 fis, clone PL | 1 | 0.23 | upregulate stage |
| 445770 | AL119499 | Hs.13285 | neuronal potassium channel alpha subunit | 1 | 1 | upregulate stage |
| 445778 | AA196443 | Hs.86043 | Homo sapiens cDNA FLJ13558 fis, clone PL | 9.7 | 0.07 | upregulate stage |
| 445787 | AI253167 | Hs.145395 | ESTs, Weakly similar to ALUC_HUMAN !!!! | 3.1 | 0.13 | upregulate stage |
| 445814 | H92020 | Hs.101624 | ESTs | 1 | 1 | upregulate stage |
| 445818 | BE045321 | Hs.136017 | ESTs | 1 | 1 | upregulate stage |
| 445829 | AI452457 | Hs.145526 | ESTs | 1 | 0.37 | upregulate stage |
| 445832 | AI261545 | | gb: qz30a07.x1 NCI_CGAP_Kid11 Homo sapien | 3.21 | 0.22 | upregulate stage |
| 445873 | AA250970 | Hs.251946 | Homo sapiens cDNA: FLJ23107 fis, clone L | 5.25 | 0.12 | upregulate stage |
| 445880 | AV655474 | Hs.131058 | ESTs | 1.05 | 0.27 | upregulate stage |
| 445883 | AF070559 | Hs.13413 | Homo sapiens clone 24463 mRNA sequence | 1 | 1 | upregulate stage |
| 445908 | R13580 | Hs.13436 | Homo sapiens clone 24425 mRNA sequence | 3.25 | 0.15 | upregulate stage |
| 445939 | BE018658 | Hs.141003 | Homo sapiens cDNA: FLJ21691 fis, clone C | 8.85 | 0.08 | upregulate stage |
| 446019 | AI362520 | Hs.94133 | ESTs | 9.75 | 0.08 | upregulate stage |
| 446054 | AB014537 | Hs.13604 | KIAA0637 gene product | 10.25 | 0.07 | upregulate stage |
| 446062 | AA211738 | Hs.282974 | ESTs, Weakly similar to transformation-r | 1 | 1 | upregulate stage |
| 446080 | AI221741 | Hs.117777 | ESTs | 9.75 | 0.09 | upregulate stage |
| 446082 | AI274139 | Hs.156452 | ESTs | 1.37 | 0.4 | upregulate stage |
| 446099 | T93096 | Hs.17126 | ESTs | 2.4 | 0.31 | upregulate stage |
| 446119 | D29527 | | gb: HUMNK667 Human epidermal keratinocyte | 1 | 1 | upregulate stage |
| 446120 | N26080 | Hs.43741 | ESTs | 1 | 0.31 | upregulate stage |
| 446126 | AW085909 | Hs.47413 | ESTs | 9.35 | 0.08 | upregulate stage |
| 446127 | AA333608 | Hs.13980 | ubiquitously transcribed tetratricopepti | 1 | 0.25 | upregulate stage |
| 446152 | AI292036 | Hs.150028 | ESTs | 4.7 | 0.12 | upregulate stage |
| 446196 | AI744888 | Hs.149470 | ESTs | 1 | 0.83 | upregulate stage |
| 446229 | AI744964 | Hs.14449 | KIAA1609 protein | 2.4 | 0.36 | upregulate stage |
| 446248 | AI283014 | Hs.149638 | ESTs | 1 | 1 | upregulate stage |
| 446269 | AW263155 | Hs.14559 | hypothetical protein FLJ10540 | 10.85 | 0.07 | upregulate stage |
| 446303 | X77244 | Hs.14732 | malic enzyme 1, NADP(+)-dependent cytos | 1 | 1 | upregulate stage |
| 446312 | BE087853 | | gb: QV1-BT0681-290400-181-h05 BT0681 Homo | 11.75 | 0.06 | upregulate stage |
| 446332 | AK001635 | Hs.14838 | hypothetical protein FLJ10773 | 6.45 | 0.1 | upregulate stage |
| 446356 | AI816736 | Hs.14896 | DHHC1 protein | 8.9 | 0.08 | upregulate stage |
| 446362 | AW612481 | Hs.255914 | ESTs | 7.6 | 0.1 | upregulate stage |
| 446398 | AI681317 | Hs.150074 | ESTs | 1 | 1 | upregulate stage |
| 446411 | AI298828 | Hs.153439 | ESTs | 1 | 0.37 | upregulate stage |
| 446474 | AI301227 | Hs.150186 | ESTs | 3.35 | 0.13 | upregulate stage |
| 446501 | AI302616 | Hs.150819 | ESTs | 4.25 | 0.12 | upregulate stage |
| 446507 | AA352554 | Hs.15164 | nuclear DNA-binding protein | 11.25 | 0.06 | upregulate stage |
| 446526 | H89616 | Hs.296290 | Homo sapiens cDNA FLJ13357 fis, clone PL | 10.25 | 0.07 | upregulate stage |
| 446555 | AV659046 | Hs.201847 | ESTs | 1 | 1 | upregulate stage |
| 446577 | AB040933 | Hs.15420 | KIAA1500 protein | 1 | 0.51 | upregulate stage |
| 446629 | AI436046 | Hs.156148 | Homo sapiens cDNA: FLJ23082 fis, clone L | 1 | 0.25 | upregulate stage |
| 446636 | AC002563 | Hs.15767 | citron (rho-interacting, serine/threonin | 2.54 | 0.28 | upregulate stage |
| 446682 | AW205632 | Hs.211198 | ESTs | 4 | 0.18 | upregulate stage |
| 446701 | AK001621 | Hs.15921 | hypothetical protein FLJ10759 | 1.32 | 0.69 | upregulate stage |
| 446718 | AV660019 | Hs.282676 | ESTs | 1 | 1 | upregulate stage |
| 446719 | W39500 | Hs.47305 | ESTs, Weakly similar to LONN_HUMAN MITOC | 9.65 | 0.07 | upregulate stage |
| 446720 | AI439136 | Hs.140546 | ESTs | 4.55 | 0.12 | upregulate stage |
| 446765 | AV660348 | Hs.282688 | ESTs | 1 | 0.91 | upregulate stage |
| 446771 | AA128965 | Hs.60679 | TATA box binding protein (TBP)-associate | 11.2 | 0.06 | upregulate stage |
| 446821 | W03766 | Hs.301482 | ESTs | 8.9 | 0.09 | upregulate stage |
| 446830 | BE179030 | Hs.64239 | Human DNA sequence from clone RP5-1174N9 | 10.65 | 0.07 | upregulate stage |
| 446839 | BE091926 | Hs.16244 | mitotic spindle coiled-coil related prot | 14.05 | 0.05 | upregulate stage |
| 446853 | AV660630 | Hs.87627 | disrupter of silencing 10 | 9.7 | 0.09 | upregulate stage |
| 446880 | AI811807 | Hs.108646 | Homo sapiens cDNA FLJ12534 fis, clone NT | 11.05 | 0.06 | upregulate stage |
| 446922 | BE175605 | | gb: RC5-HT0580-100500-022-H07 HT0580 Homo | 2.75 | 0.16 | upregulate stage |
| 446950 | AA305800 | Hs.293454 | ESTs, Weakly similar to Similarity to Ye | 9.6 | 0.06 | upregulate stage |
| 446988 | AW339533 | Hs.272108 | ESTs | 1 | 0.29 | upregulate stage |
| 446989 | AK001898 | Hs.16740 | hypothetical protein FLJ11036 | 1.75 | 0.51 | upregulate stage |
| 447052 | AV661911 | Hs.282735 | ESTs | 1 | 1 | upregulate stage |
| 447065 | AI829014 | Hs.158678 | ESTs | 1 | 0.25 | upregulate stage |
| 447069 | AI359927 | Hs.157722 | ESTs | 1 | 0.4 | upregulate stage |
| 447078 | AW885727 | Hs.301570 | ESTs | 4.4 | 0.13 | upregulate stage |
| 447080 | AI418781 | Hs.300144 | ESTs | 1 | 0.31 | upregulate stage |
| 447081 | Y13896 | Hs.17287 | potassium inwardly-rectifying channel, s | 3.55 | 0.12 | upregulate stage |
| 447118 | AB014599 | Hs.17411 | KIAA0699 protein | 10.15 | 0.07 | upregulate stage |
| 447153 | AA805202 | Hs.173912 | eukaryotic translation initiation factor | 6.2 | 0.12 | upregulate stage |
| 447154 | H52284 | Hs.293545 | ESTs | 1 | 0.24 | upregulate stage |
| 447159 | AI685286 | Hs.280386 | EST | 1.25 | 0.25 | upregulate stage |
| 447215 | BE617056 | Hs.283000 | ESTs | 2.4 | 0.16 | upregulate stage |
| 447228 | AW192200 | Hs.158188 | ESTs | 1 | 0.29 | upregulate stage |
| 447258 | BE047911 | | gb: tz44a05.y1 NCI_CGAP_Brn52 Homo sapien | 1.15 | 0.23 | upregulate stage |
| 447286 | AW197097 | Hs.183858 | transcriptional intermediary factor 1 | 1 | 1 | upregulate stage |
| 447289 | AW247017 | Hs.36978 | melanoma antigen, family A, 3 | 1 | 1 | upregulate stage |
| 447334 | AA515032 | Hs.91109 | ESTs | 9.15 | 0.08 | upregulate stage |
| 447342 | AI199268 | Hs.19322 | ESTs | 5.95 | 0.09 | upregulate stage |
| 447343 | AA256641 | Hs.236894 | ESTs, Highly similar to LRP1_HUMAN LOW-D | 2.11 | 0.33 | upregulate stage |
| 447376 | AI376747 | | gb: tc35h05.x1 Soares_total_fetus_Nb2HF8_{—} | 1 | 0.33 | upregulate stage |
| 447397 | BE247676 | Hs.18442 | E-1 enzyme | 5.3 | 0.14 | upregulate stage |
| 447430 | AI742989 | Hs.206112 | ESTs | 3.65 | 0.13 | upregulate stage |
| 447444 | AK000318 | Hs.18616 | hypothetical protein FLJ20311 | 9.2 | 0.08 | upregulate stage |
| 447519 | U46258 | Hs.23448 | ESTs | 14.4 | 0.05 | upregulate stage |
| 447522 | BE143888 | | gb: MR0-HT0165-081199-001-b04 HT0165 Homo | 1.7 | 0.18 | upregulate stage |
| 447578 | AA912347 | Hs.136585 | ESTs | 1.5 | 0.3 | upregulate stage |
| 447606 | AI588954 | Hs.170995 | ESTs | 2.7 | 0.16 | upregulate stage |
| 447688 | N87079 | Hs.19236 | NADH dehydrogenase (ubiquinone) 1 beta s | 4.55 | 0.12 | upregulate stage |
| 447701 | BE619526 | Hs.255527 | ESTs, Weakly similar to unnamed protein | 1.63 | 0.46 | upregulate stage |
| 447741 | AI421737 | Hs.167253 | ESTs | 1 | 1 | upregulate stage |
| 447748 | AI422023 | Hs.161338 | ESTs | 3.9 | 0.11 | upregulate stage |
| 447827 | U73727 | Hs.19718 | protein tyrosine phosphatase, receptor t | 1.44 | 0.59 | upregulate stage |
| 447881 | BE620886 | Hs.23037 | ESTs | 12.15 | 0.06 | upregulate stage |
| 447963 | AI452973 | Hs.165900 | ESTs, Weakly similar to ALUC_HUMAN !!!! | 8.9 | 0.08 | upregulate stage |
| 447977 | AI457097 | Hs.255906 | ESTs | 1 | 1 | upregulate stage |
| 447978 | AI457098 | Hs.280848 | ESTs | 1 | 1 | upregulate stage |
| 447982 | H22953 | Hs.137551 | ESTs | 4.25 | 0.13 | upregulate stage |
| 448032 | AW511770 | Hs.246868 | ESTs | 1 | 1 | upregulate stage |
| 448045 | AJ297436 | Hs.20166 | prostate stem cell antigen | 2.22 | 0.29 | upregulate stage |
| 448058 | AI458998 | Hs.170424 | ESTs | 1 | 0.51 | upregulate stage |
| 448062 | AW295923 | Hs.255472 | ESTs | 5.9 | 0.08 | upregulate stage |
| 448138 | AW847925 | Hs.170736 | ESTs | 1 | 1 | upregulate stage |
| 448154 | AL120320 | Hs.203230 | ESTs | 9.85 | 0.07 | upregulate stage |
| 448165 | NM_005591 | Hs.202379 | meiotic recombination (S. cerivisiae) 11 | 7.3 | 0.09 | upregulate stage |
| 448168 | AW605999 | Hs.22549 | hypothetical protein FLJ12799 | 1 | 0.77 | upregulate stage |
| 448236 | AA890449 | Hs.20766 | oxysterol 7alpha-hydroxylase | 1 | 0.47 | upregulate stage |
| 448256 | BE614149 | Hs.20814 | CGI-27 protein | 11.95 | 0.07 | upregulate stage |
| 448289 | AW390251 | Hs.202402 | ESTs | 1 | 0.47 | upregulate stage |
| 448356 | AL120837 | Hs.20993 | high-glucose-regulated protein 8 | 11 | 0.07 | upregulate stage |
| 448357 | N20169 | Hs.108923 | ESTs | 1.34 | 0.61 | upregulate stage |
| 448408 | AA322866 | Hs.21107 | neuroligin | 1.7 | 0.24 | upregulate stage |
| 448455 | AI252625 | Hs.269860 | ESTs | 8.8 | 0.09 | upregulate stage |
| 448459 | AW069838 | Hs.171055 | ESTs | 1 | 0.27 | upregulate stage |
| 448464 | AI522053 | Hs.196093 | ESTs | 10.35 | 0.06 | upregulate stage |
| 448468 | BE550361 | Hs.171072 | ESTs | 1 | 1 | upregulate stage |
| 448502 | AW805285 | Hs.239699 | ESTs | 9.3 | 0.08 | upregulate stage |
| 448552 | AW973653 | Hs.20104 | hypothetical protein FLJ00052 | 4.75 | 0.13 | upregulate stage |
| 448556 | AW885606 | Hs.5064 | ESTs | 9.8 | 0.08 | upregulate stage |
| 448569 | BE382657 | Hs.21486 | signal transducer and activator of trans | 2.14 | 0.35 | upregulate stage |
| 448632 | BE614269 | | gb: 601504311T1 NIH_MGC_71 Homo sapiens c | 1 | 1 | upregulate stage |
| 448643 | AI557531 | | gb: pt2.1-06.D06.r tumor2 Homo sapiens cD | 3.6 | 0.14 | upregulate stage |
| 448649 | T94590 | Hs.222855 | ESTs | 1.95 | 0.21 | upregulate stage |
| 448663 | BE614599 | Hs.106823 | H. sapiens gene from PAC 426I6, similar t | 4.3 | 0.12 | upregulate stage |
| 448680 | AW245890 | Hs.21753 | JM5 protein | 0.97 | 0.93 | upregulate stage |
| 448725 | AA193251 | Hs.40289 | ESTs | 2.6 | 0.19 | upregulate stage |
| 448729 | BE614535 | Hs.138580 | ESTs, Weakly similar to ALU5_HUMAN ALU S | 3.25 | 0.16 | upregulate stage |
| 448743 | AB032962 | Hs.21896 | KIAA1136 protein | 1.9 | 0.19 | upregulate stage |
| 448826 | AI580252 | Hs.293246 | ESTs, Weakly similar to putative p150 [H | 1.78 | 0.44 | upregulate stage |
| 448914 | AI927656 | Hs.196459 | ESTs | 2.75 | 0.19 | upregulate stage |
| 448946 | AI652855 | Hs.155796 | ESTs | 9.7 | 0.07 | upregulate stage |
| 448958 | AB020651 | Hs.22653 | KIAA0844 protein | 1 | 0.18 | upregulate stage |
| 448974 | AL049390 | Hs.22689 | Homo sapiens mRNA; cDNA DKFZp586O1318 (f | 5.85 | 0.11 | upregulate stage |
| 448979 | AI611378 | Hs.192610 | ESTs | 1 | 1 | upregulate stage |
| 449008 | AW578003 | Hs.22826 | tropomodulin 3 (ubiquitous) | 5.2 | 0.11 | upregulate stage |
| 449032 | AA045573 | Hs.22900 | nuclear factor (erythroid-derived 2)-lik | 1 | 0.33 | upregulate stage |
| 449053 | AI625777 | Hs.270344 | ESTs | 5.73 | 0.12 | upregulate stage |
| 449057 | AB037784 | Hs.22941 | KIAA1363 protein | 9.25 | 0.07 | upregulate stage |
| 449148 | AW836677 | Hs.287564 | Homo sapiens cDNA FLJ13345 fis, clone OV | 7.2 | 0.09 | upregulate stage |
| 449203 | AI634578 | Hs.282121 | ESTs | 7 | 0.1 | upregulate stage |
| 449207 | AL044222 | Hs.23255 | nucleoporin 155 kD | 2.34 | 0.36 | upregulate stage |
| 449219 | AI637581 | Hs.195012 | ESTs | 1 | 1 | upregulate stage |
| 449230 | BE613348 | Hs.23348 | S-phase kinase-associated protein 2 (p45 | 3.08 | 0.25 | upregulate stage |
| 449246 | AW411209 | Hs.23363 | hypothetical protein FLJ10983 | 4.79 | 0.16 | upregulate stage |
| 449318 | AW236021 | Hs.108788 | ESTs, Weakly similar to zeste [D. melanog | 2.8 | 0.16 | upregulate stage |
| 449328 | AI962493 | Hs.197647 | ESTs | 2.55 | 0.17 | upregulate stage |
| 449343 | AI151418 | Hs.272458 | protein phosphatase 3 (formerly 2B), cat | 4.75 | 0.12 | upregulate stage |
| 449344 | AI640355 | | gb: wa17c04.x1 NCI_CGAP_Kid11 Homo sapien | 2.1 | 0.22 | upregulate stage |
| 449351 | AW016537 | Hs.200760 | ESTs | 2.45 | 0.14 | upregulate stage |
| 449370 | AK002114 | Hs.23495 | hypothetical protein FLJ11252 | 1.55 | 0.14 | upregulate stage |
| 449424 | AW448937 | Hs.197030 | ESTs | 4.05 | 0.12 | upregulate stage |
| 449425 | AW103433 | Hs.195684 | ESTs | 4.6 | 0.12 | upregulate stage |
| 449434 | AW294858 | Hs.197641 | ESTs | 1 | 0.29 | upregulate stage |
| 449437 | AI702038 | Hs.100057 | Homo sapiens cDNA: FLJ22902 fis, clone K | 2.38 | 0.34 | upregulate stage |
| 449474 | AA019344 | Hs.2055 | ubiquitin-activating enzyme E1 (A1S9T an | 5.9 | 0.12 | upregulate stage |
| 449523 | NM_000579 | Hs.54443 | chemokine (C-C motif) receptor 5 | 6.45 | 0.1 | upregulate stage |
| 449528 | H63337 | Hs.38178 | Homo sapiens cDNA: FLJ23468 fis, clone H | 2.85 | 0.18 | upregulate stage |
| 449565 | AI824925 | Hs.197066 | ESTs | 1 | 1 | upregulate stage |
| 449568 | AL157479 | Hs.23740 | KIAA1598 protein | 10.15 | 0.06 | upregulate stage |
| 449618 | AI076459 | Hs.14366 | Homo sapiens cDNA FLJ12819 fis, clone NT | 11.7 | 0.06 | upregulate stage |
| 449639 | AA001968 | Hs.59956 | ESTs, Highly similar to MGR7_HUMAN METAB | 1 | 1 | upregulate stage |
| 449666 | AA002047 | | gb: zh84e05.r1 Soares_fetal_liver_spleen_{—} | 1.85 | 0.33 | upregulate stage |
| 449704 | AK000733 | Hs.23900 | GTPase activating protein | 2.82 | 0.3 | upregulate stage |
| 449722 | BE280074 | Hs.23960 | cyclin B1 | 6.44 | 0.12 | upregulate stage |
| 449764 | N93104 | Hs.54895 | ESTs, Weakly similar to ZNF91L [H. sapien | 1 | 1 | upregulate stage |
| 449784 | AW161319 | Hs.12915 | ESTs | 6.25 | 0.11 | upregulate stage |
| 449829 | N51440 | Hs.47261 | ESTs | 1 | 0.57 | upregulate stage |
| 449843 | R85337 | Hs.24030 | solute carrier family 31 (copper transpo | 10.2 | 0.07 | upregulate stage |
| 449892 | N73608 | Hs.50309 | ESTs | 6.5 | 0.1 | upregulate stage |
| 449894 | AK001578 | Hs.24129 | hypothetical protein FLJ10716 | 4.55 | 0.12 | upregulate stage |
| 449919 | AI674685 | Hs.200141 | ESTs | 5.3 | 0.11 | upregulate stage |
| 450020 | AI680684 | Hs.282219 | ESTs | 1 | 1 | upregulate stage |
| 450033 | R43010 | Hs.269452 | ESTs, Weakly similar to JH0148 nucleolin | 1 | 0.65 | upregulate stage |
| 450063 | AI681509 | Hs.277133 | ESTs | 4.2 | 0.17 | upregulate stage |
| 450083 | AA131795 | Hs.142001 | ESTs | 3.9 | 0.16 | upregulate stage |
| 450116 | AA005355 | Hs.222882 | ESTs | 1 | 1 | upregulate stage |
| 450121 | AL040174 | Hs.288927 | Homo sapiens cDNA: FLJ22944 fis, clone K | 1 | 1 | upregulate stage |
| 450135 | AI810816 | Hs.201142 | ESTs | 4.95 | 0.14 | upregulate stage |
| 450144 | T63961 | Hs.301851 | ESTs | 2.75 | 0.13 | upregulate stage |
| 450149 | AW969781 | Hs.293440 | ESTs, Moderately similar to ZIC2 protein | 3.75 | 0.14 | upregulate stage |
| 450151 | AI088196 | Hs.295233 | ESTs | 2.51 | 0.28 | upregulate stage |
| 450152 | AI138635 | Hs.22968 | ESTs | 2.45 | 0.15 | upregulate stage |
| 450195 | AA007352 | Hs.256042 | ESTs | 4.1 | 0.14 | upregulate stage |
| 450221 | AA328102 | Hs.24641 | cytoskeleton associated protein 2 | 1 | 0.3 | upregulate stage |
| 450238 | T89693 | Hs.138777 | ESTs | 12.2 | 0.07 | upregulate stage |
| 450257 | AW820313 | | gb: QV2-ST0296-150200-028-d02 ST0296 Homo | 1 | 1 | upregulate stage |
| 450313 | AI038989 | Hs.24809 | hypothetical protein FLJ10826 | 4.35 | 0.15 | upregulate stage |
| 450314 | AA574309 | Hs.283402 | TCR eta | 10.1 | 0.07 | upregulate stage |
| 450350 | T97817 | Hs.174880 | ESTs | 3.65 | 0.1 | upregulate stage |
| 450411 | D61167 | Hs.202156 | ESTs | 1 | 0.67 | upregulate stage |
| 450447 | AF212223 | Hs.25010 | hypothetical protein P15-2 | 10.75 | 0.07 | upregulate stage |
| 450448 | D54299 | Hs.36244 | ESTs | 1 | 1 | upregulate stage |
| 450449 | AI696596 | Hs.202068 | ESTs | 1 | 1 | upregulate stage |
| 450506 | NM_004460 | Hs.418 | fibroblast activation protein, alpha | 11.45 | 0.05 | upregulate stage |
| 450573 | AW964334 | | gb: EST376407 MAGE resequences, MAGH Homo | 1.2 | 0.2 | upregulate stage |
| 450628 | AW382884 | Hs.204715 | ESTs | 4.95 | 0.13 | upregulate stage |
| 450636 | AI703076 | Hs.201959 | ESTs | 1 | 0.69 | upregulate stage |
| 450655 | AI707846 | Hs.279860 | hypothetical protein FLJ20030 | 1 | 1 | upregulate stage |
| 450664 | AA808358 | Hs.36830 | ESTs | 1 | 0.34 | upregulate stage |
| 450680 | AF131784 | Hs.25318 | Homo sapiens clone 25194 mRNA sequence | 9.51 | 0.09 | upregulate stage |
| 450722 | AI732318 | Hs.101120 | ESTs | 1 | 0.87 | upregulate stage |
| 450751 | AI733251 | Hs.126853 | ESTs, Weakly similar to JU0033 hypotheti | 1 | 1 | upregulate stage |
| 450772 | BE326391 | Hs.280146 | ESTs, Weakly similar to JU0033 hypotheti | 1 | 1 | upregulate stage |
| 450800 | BE395161 | Hs.243963 | ESTs, Weakly similar to ALU5_HUMAN ALU S | 8.7 | 0.08 | upregulate stage |
| 450824 | R09055 | Hs.269204 | ESTs | 3.03 | 0.22 | upregulate stage |
| 450832 | AW970602 | Hs.105421 | ESTs | 6.15 | 0.08 | upregulate stage |
| 450870 | AA011471 | | gb: zi01h08.r1 Soares_fetal_liver_spleen_{—} | 1.15 | 0.23 | upregulate stage |
| 450937 | R49131 | Hs.26267 | ATP-dependant interferon response protei | 9.75 | 0.08 | upregulate stage |
| 450983 | AA305384 | Hs.25740 | ERO1 (S. cerivisiae)-like | 3.32 | 0.26 | upregulate stage |
| 451052 | AA281504 | Hs.24444 | ESTs, Moderately similar to ALUE_HUMAN ! | 9.25 | 0.08 | upregulate stage |
| 451067 | BE172186 | Hs.180789 | S164 protein | 2.8 | 0.21 | upregulate stage |
| 451088 | AA015600 | Hs.82415 | ESTs | 1 | 0.32 | upregulate stage |
| 451094 | AI949825 | Hs.260395 | ESTs | 4.45 | 0.14 | upregulate stage |
| 451096 | BE383234 | Hs.25925 | Homo sapiens clone 23860 mRNA sequence | 4.15 | 0.14 | upregulate stage |
| 451126 | H30600 | Hs.40910 | ESTs | 1 | 1 | upregulate stage |
| 451161 | AA211329 | Hs.26006 | hypothetical protein FLJ10559 | 2.15 | 0.16 | upregulate stage |
| 451166 | T98171 | Hs.185675 | ESTs | 9.26 | 0.08 | upregulate stage |
| 451222 | AA018386 | Hs.64341 | ESTs | 1 | 0.36 | upregulate stage |
| 451225 | AI433694 | Hs.293608 | ESTs | 9.19 | 0.08 | upregulate stage |
| 451228 | AI767166 | Hs.207025 | ESTs | 1 | 1 | upregulate stage |
| 451246 | AW189232 | Hs.39140 | cutaneous T-cell lymphoma tumor antigen | 7.35 | 0.11 | upregulate stage |
| 451266 | AA016292 | Hs.290849 | ESTs | 1 | 0.33 | upregulate stage |
| 451276 | AW294386 | Hs.236533 | ESTs, Highly similar to dJ742C19.2 [H. sa | 1 | 1 | upregulate stage |
| 451277 | AK001123 | Hs.26176 | hypothetical protein FLJ10261 | 11.7 | 0.06 | upregulate stage |
| 451291 | R39288 | Hs.6702 | ESTs | 1 | 1 | upregulate stage |
| 451326 | AW296946 | Hs.300967 | ESTs | 10.55 | 0.07 | upregulate stage |
| 451347 | AI288679 | Hs.101139 | ESTs | 1 | 1 | upregulate stage |
| 451359 | H85334 | | gb: ys90e05.r1 Soares retina N2b5HR Homo | 2.7 | 0.15 | upregulate stage |
| 451365 | AI791783 | | gb: op20h10.y5 NCI_CGAP_Co12 Homo sapiens | 8.9 | 0.09 | upregulate stage |
| 451386 | AB029006 | Hs.26334 | spastic paraplegia 4 (autosomal dominant | 2.45 | 0.19 | upregulate stage |
| 451440 | AA017599 | Hs.293817 | ESTs | 1 | 1 | upregulate stage |
| 451487 | AA018072 | | gb: ze51g02.r1 Soares retina N2b4HR Homo | 5.7 | 0.1 | upregulate stage |
| 451492 | AA018119 | Hs.297824 | ESTs, Highly similar to CIK1_HUMAN VOLTA | 1 | 1 | upregulate stage |
| 451495 | H86887 | | gb: yt07a01.r1 Soares retina N2b5HR Homo | 4.25 | 0.13 | upregulate stage |
| 451535 | AW970577 | | gb: EST382658 MAGE resequences, MAGK Homo | 6.8 | 0.12 | upregulate stage |
| 451553 | AA018454 | Hs.269211 | ESTs, Weakly similar to B34087 hypotheti | 1 | 1 | upregulate stage |
| 451562 | H04150 | Hs.107708 | ESTs | 4.65 | 0.11 | upregulate stage |
| 451580 | AW138195 | Hs.184326 | CDC10 (cell division cycle 10, S. cerevi | 1 | 0.42 | upregulate stage |
| 451592 | AI805416 | Hs.213897 | ESTs | 2.8 | 0.17 | upregulate stage |
| 451651 | AI097337 | Hs.88977 | hypothetical protein dJ511E16.2 | 1 | 0.18 | upregulate stage |
| 451658 | AW195351 | Hs.250520 | ESTs | 9.55 | 0.07 | upregulate stage |
| 451684 | AF216751 | Hs.26813 | CDA14 | 3.7 | 0.15 | upregulate stage |
| 451690 | AW451469 | Hs.209990 | ESTs | 10.86 | 0.07 | upregulate stage |
| 451724 | AI903765 | | gb: UI-BT037-301298-102 BT037 Homo sapien | 8.85 | 0.09 | upregulate stage |
| 451743 | AW074266 | Hs.23071 | ESTs | 2.17 | 0.35 | upregulate stage |
| 451794 | AA019799 | Hs.111911 | ESTs | 1 | 1 | upregulate stage |
| 451844 | T61430 | | gb: yc06a03.s1 Stratagene lung (937210) H | 6.5 | 0.11 | upregulate stage |
| 451903 | W19617 | Hs.261003 | ESTs, Moderately similar to B34087 hypot | 2.2 | 0.21 | upregulate stage |
| 451914 | AI822115 | Hs.270618 | ESTs, Weakly similar to KIAA0822 protein | 11.67 | 0.07 | upregulate stage |
| 451938 | AI354355 | Hs.16697 | down-regulator of transcription 1, TBP-b | 11.65 | 0.06 | upregulate stage |
| 451939 | U80456 | Hs.27311 | single-minded (Drosophila) homolog 2 | 1 | 0.95 | upregulate stage |
| 451971 | AA021185 | Hs.226306 | ESTs | 1 | 1 | upregulate stage |
| 451998 | AW594129 | Hs.213666 | ESTs | 1 | 0.26 | upregulate stage |
| 452028 | AK001859 | Hs.27595 | hypothetical protein FLJ10997 | 1 | 0.21 | upregulate stage |
| 452036 | NM_003966 | Hs.27621 | sema domain, seven thrombospondin repeat | 1.76 | 0.41 | upregulate stage |
| 452099 | BE612992 | Hs.27931 | hypothetical protein FLJ10607 similar to | 8.9 | 0.07 | upregulate stage |
| 452122 | AF216833 | Hs.1710 | ATP-binding cassette, sub-family B (MDR/ | 1 | 0.47 | upregulate stage |
| 452163 | AI863140 | | gb: tz43h12.x1 NCI_CGAP_Bm52 Homo sapien | 1 | 0.2 | upregulate stage |
| 452179 | H16725 | Hs.27463 | ESTs | 3 | 0.13 | upregulate stage |
| 452198 | AI097560 | Hs.61210 | ESTs | 1 | 0.28 | upregulate stage |
| 452206 | AW340281 | Hs.33074 | ESTs, Moderately similar to ALU1_HUMAN A | 12.4 | 0.07 | upregulate stage |
| 452234 | AW084176 | Hs.223296 | ESTs | 6.8 | 0.09 | upregulate stage |
| 452240 | AI591147 | Hs.61232 | ESTs | 3.75 | 0.07 | upregulate stage |
| 452247 | AL137432 | Hs.28564 | hypothetical protein DKFZp761E1824 | 3.9 | 0.15 | upregulate stage |
| 452250 | BE618654 | Hs.28607 | hypothetical protein A-211C6.1 | 8.75 | 0.09 | upregulate stage |
| 452256 | AK000933 | Hs.28661 | Homo sapiens cDNA FLJ10071 fis, clone HE | 5.2 | 0.09 | upregulate stage |
| 452266 | AI767250 | Hs.165240 | ESTs | 10.45 | 0.06 | upregulate stage |
| 452277 | AL049013 | Hs.28783 | KIAA1223 protein | 8.9 | 0.05 | upregulate stage |
| 452281 | T93500 | Hs.28792 | Homo sapiens cDNA FLJ11041 fis, clone PL | 8.2 | 0.04 | upregulate stage |
| 452291 | AF015592 | Hs.28853 | CDC7 (cell division cycle 7, S. cerevisi | 3.5 | 0.13 | upregulate stage |
| 452328 | AA805679 | Hs.61271 | ESTs | 3.5 | 0.14 | upregulate stage |
| 452331 | AA598509 | Hs.29117 | H. sapiens mRNA for pur alpha extended 3′ | 11.75 | 0.07 | upregulate stage |
| 452345 | AA293279 | Hs.29173 | hypothetical protein FLJ20515 | 1.08 | 0.73 | upregulate stage |
| 452367 | U71207 | Hs.29279 | eyes absent (Drosophila) homolog 2 | 10.15 | 0.07 | upregulate stage |
| 452401 | NM_007115 | Hs.29352 | tumor necrosis factor, alpha-induced pro | 1 | 0.17 | upregulate stage |
| 452404 | AW450675 | Hs.212709 | ESTs | 3.63 | 0.2 | upregulate stage |
| 452430 | AF118083 | Hs.29494 | PRO1912 protein | 1 | 0.41 | upregulate stage |
| 452436 | BE077546 | Hs.31447 | ESTs | 10 | 0.07 | upregulate stage |
| 452457 | AW062499 | | gb: MR0-CT0065-100899-001-d02 CT0065 Homo | 1 | 0.13 | upregulate stage |
| 452461 | N78223 | Hs.108106 | transcription factor | 8.1 | 0.06 | upregulate stage |
| 452518 | AA280722 | Hs.24758 | ESTs | 9.3 | 0.08 | upregulate stage |
| 452519 | BE006701 | | gb: RC0-BN0132-270300-021-a03 BN0132 Homo | 1 | 0.19 | upregulate stage |
| 452524 | AW136499 | Hs.29796 | Homo sapiens mRNA; cDNA DKFZp434D1319 (f | 1 | 0.45 | upregulate stage |
| 452531 | AA429462 | Hs.293946 | ESTs | 2.94 | 0.22 | upregulate stage |
| 452547 | AA335295 | Hs.74120 | adipose specific 2 | 1.51 | 0.53 | upregulate stage |
| 452560 | BE077084 | | gb: RC5-BT0603-220200-013-C07 BT0603 Homo | 5.35 | 0.11 | upregulate stage |
| 452571 | W31518 | Hs.34665 | ESTs | 2.55 | 0.11 | upregulate stage |
| 452607 | AI160029 | Hs.61438 | ESTs | 4.75 | 0.11 | upregulate stage |
| 452677 | BE167202 | Hs.212065 | ESTs | 1 | 0.32 | upregulate stage |
| 452680 | AW138410 | Hs.45051 | ESTs | 1 | 1 | upregulate stage |
| 452724 | R84810 | Hs.30464 | cyclin E2 | 1 | 0.27 | upregulate stage |
| 452738 | AL133800 | | gb: DKFZp761A0614_r1 761 (synonym: hamy2) | 3.45 | 0.15 | upregulate stage |
| 452741 | BE392914 | Hs.30503 | Homo sapiens cDNA FLJ11344 fis, clone PL | 3.05 | 0.16 | upregulate stage |
| 452747 | BE153855 | Hs.61460 | ESTs | 2.54 | 0.28 | upregulate stage |
| 452761 | BE244742 | Hs.30532 | CGI-77 protein | 3.85 | 0.14 | upregulate stage |
| 452825 | AI921523 | | gb: wo26d09.x1 NCI_CGAP_Gas4 Homo sapiens | 1 | 1 | upregulate stage |
| 452831 | AW864089 | Hs.135145 | ESTs | 2.4 | 0.19 | upregulate stage |
| 452846 | AA082160 | Hs.204295 | ESTs | 8.9 | 0.08 | upregulate stage |
| 452850 | H23230 | Hs.22481 | ESTs | 4.75 | 0.14 | upregulate stage |
| 452859 | AI300555 | Hs.288158 | Homo sapiens cDNA: FLJ23591 fis, clone L | 9.15 | 0.08 | upregulate stage |
| 452862 | AW378065 | Hs.8687 | ESTs | 5.95 | 0.07 | upregulate stage |
| 452899 | M96739 | Hs.30956 | Human NSCL-1 mRNA sequence | 1.04 | 0.9 | upregulate stage |
| 452902 | AI926501 | Hs.249729 | ESTs | 6.8 | 0.1 | upregulate stage |
| 452909 | NM_015368 | Hs.30985 | pannexin 1 | 5.6 | 0.1 | upregulate stage |
| 452931 | AW190011 | Hs.158006 | hypothetical protein | 1 | 0.53 | upregulate stage |
| 452934 | AA581322 | Hs.4213 | ESTs | 1.44 | 0.55 | upregulate stage |
| 452956 | AW003578 | Hs.231872 | ESTs | 1 | 0.22 | upregulate stage |
| 452974 | BE090803 | Hs.61506 | ESTs | 1.75 | 0.18 | upregulate stage |
| 453011 | N62952 | Hs.46473 | ESTs | 1 | 1 | upregulate stage |
| 453050 | AW136479 | Hs.224046 | ESTs | 1 | 0.39 | upregulate stage |
| 453074 | AA031813 | Hs.271880 | ESTs | 1 | 1 | upregulate stage |
| 453076 | AI978583 | Hs.232161 | ESTs | 3.75 | 0.14 | upregulate stage |
| 453123 | AI953718 | Hs.221849 | ESTs | 6.6 | 0.11 | upregulate stage |
| 453134 | AA032211 | Hs.118493 | ESTs | 1.68 | 0.42 | upregulate stage |
| 453135 | T07866 | Hs.31834 | Homo sapiens clone 25129 mRNA sequence | 1 | 1 | upregulate stage |
| 453137 | AI954733 | Hs.223640 | ESTs | 1 | 0.51 | upregulate stage |
| 453144 | AW268807 | Hs.61646 | ESTs | 1 | 0.26 | upregulate stage |
| 453153 | N53893 | Hs.24360 | ESTs | 5 | 0.13 | upregulate stage |
| 453156 | BE463762 | Hs.223784 | ESTs | 2.8 | 0.15 | upregulate stage |
| 453204 | R10799 | Hs.191990 | ESTs | 9.5 | 0.05 | upregulate stage |
| 453228 | AW628325 | Hs.232327 | ESTs | 1 | 1 | upregulate stage |
| 453274 | AA018511 | Hs.32769 | Homo sapiens mRNA full length insert cDN | 1 | 1 | upregulate stage |
| 453293 | AA382267 | Hs.10653 | ESTs | 8.4 | 0.09 | upregulate stage |
| 453321 | AI984381 | Hs.232521 | ESTs | 6.7 | 0.1 | upregulate stage |
| 453329 | T97205 | Hs.17998 | ESTs | 8.9 | 0.08 | upregulate stage |
| 453389 | BE273648 | Hs.32963 | cadherin 6, type 2, K-cadherin (fetal ki | 1 | 0.18 | upregulate stage |
| 453437 | H10751 | Hs.79981 | Human clone 23560 mRNA sequence | 1 | 0.83 | upregulate stage |
| 453450 | AW797627 | Hs.89474 | ADP-ribosylation factor 6 | 7.09 | 0.08 | upregulate stage |
| 453459 | BE047032 | Hs.257789 | ESTs | 2.35 | 0.3 | upregulate stage |
| 453476 | AI640500 | Hs.24633 | SAM domain, SH3 domain and nuclear local | 2.75 | 0.16 | upregulate stage |
| 453651 | AA971698 | Hs.159397 | x 010 protein | 8.95 | 0.08 | upregulate stage |
| 453653 | AW505554 | Hs.300284 | ESTs | 4.6 | 0.1 | upregulate stage |
| 453775 | NM_002916 | Hs.35120 | replication factor C (activator 1) 4 (37 | 3.4 | 0.1 | upregulate stage |
| 453776 | R15749 | Hs.31677 | ESTs | 1 | 1 | upregulate stage |
| 453846 | AL157586 | | gb: DKFZp761H0216_r1 761 (synonym: hamy2) | 1 | 0.95 | upregulate stage |
| 453884 | AA355925 | Hs.36232 | KIAA0186 gene product | 10.25 | 0.06 | upregulate stage |
| 453900 | AW003582 | Hs.226414 | ESTs, Weakly similar to ALU8_HUMAN ALU S | 4.75 | 0.12 | upregulate stage |
| 453913 | AW004683 | Hs.233502 | ESTs | 3.65 | 0.14 | upregulate stage |
| 453925 | AW021088 | Hs.181614 | ESTs | 3.7 | 0.13 | upregulate stage |
| 453931 | AL121278 | Hs.25144 | ESTs | 3.45 | 0.18 | upregulate stage |
| 453945 | NM_005171 | Hs.36908 | activating transcription factor 1 | 6.35 | 0.12 | upregulate stage |
| 454032 | W31790 | Hs.194293 | ESTs | 6.15 | 0.07 | upregulate stage |
| 454049 | AW022885 | | gb: df45e05.y1 Morton Fetal Cochlea Homo | 2.8 | 0.15 | upregulate stage |
| 454069 | AW025160 | Hs.34161 | ESTs, Moderately similar to ALU1_HUMAN A | 1 | 0.32 | upregulate stage |
| 454099 | AW062974 | | gb: IL1-ST0041-020899-001-H08 ST0041 Homo | 1 | 1 | upregulate stage |
| 454111 | AW081681 | Hs.269064 | ESTs | 2.8 | 0.18 | upregulate stage |
| 454219 | X75042 | Hs.44313 | v-rel avian reticuloendotheliosis viral | 9.4 | 0.05 | upregulate stage |
| 454259 | AL110136 | Hs.47679 | Homo sapiens mRNA: cDNA DKFZp564I112 (fr | 6.2 | 0.11 | upregulate stage |
| 454327 | BE064097 | | gb: QV3-BT0297-231199-020-h08 BT0297 Homo | 1 | 1 | upregulate stage |
| 454331 | AW372937 | | gb: QV3-BT0381-161299-042-a09 BT0381 Homo | 1 | 0.43 | upregulate stage |
| 454380 | AW858722 | | gb: RC3-CT0347-281199-011-c04 CT0347 Homo | 1 | 0.29 | upregulate stage |
| 454524 | AW857191 | | gb: RC2-CT0304-080100-011-b12 CT0304 Homo | 10.55 | 0.08 | upregulate stage |
| 454592 | AW810112 | | gb: MR4-ST0124-100400-006-e07 ST0124 Homo | 1 | 0.37 | upregulate stage |
| 454648 | AW811960 | | gb: RC2-ST0168-240300-017-f09 ST0168 Homo | 1 | 0.4 | upregulate stage |
| 454687 | AW814473 | | gb: MR3-ST0203-010200-109-c11 ST0203 Homo | 1 | 1 | upregulate stage |
| 454692 | AW813350 | | gb: MR3-ST0192-100100-024-g07 ST0192 Homo | 4.45 | 0.14 | upregulate stage |
| 454702 | BE145915 | | gb: MR0-HT0208-221299-204-h08 HT0208 Homo | 9.65 | 0.08 | upregulate stage |
| 454729 | AW817003 | | gb: QV0-ST0247-040100-081-f03 ST0247 Homo | 1 | 0.8 | upregulate stage |
| 454789 | BE156314 | | gb: QV0-HT0367-150200-114-d02 HT0367 Homo | 1 | 0.31 | upregulate stage |
| 454797 | BE161168 | | gb: PM0-HT0425-170100-002-a10 HT0425 Homo | 4.1 | 0.14 | upregulate stage |
| 454863 | AW835610 | | gb: QV4-LT0016-090200-100-c02 LT0016 Homo | 1 | 1 | upregulate stage |
| 454893 | AW837753 | | gb: CM1-LT0042-310100-112-g03 LT0042 Homo | 4.45 | 0.18 | upregulate stage |
| 454898 | AW838125 | | gb: QV2-LT0051-240300-097-e12 LT0051 Homo | 1 | 1 | upregulate stage |
| 454951 | AW847464 | | gb: RC3-CT0208-270999-021-h12 CT0208 Homo | 7.4 | 0.1 | upregulate stage |
| 454956 | AW847725 | | gb: IL3-CT0213-180200-041-H10 CT0213 Homo | 1 | 0.23 | upregulate stage |
| 455047 | AW852530 | | gb: PM1-CT0243-071099-001-g06 CT0243 Homo | 4.25 | 0.13 | upregulate stage |
| 455128 | AW861555 | | gb: RC2-CT0321-110100-013-b05 CT0321 Homo | 2.9 | 0.13 | upregulate stage |
| 455201 | AW947884 | | gb: PM1-MT0010-200300-001-g08 MT0010 Homo | 3.15 | 0.16 | upregulate stage |
| 455207 | AW994394 | | gb: RC3-BN0036-060400-014-h12 BN0036 Homo | 1 | 0.18 | upregulate stage |
| 455331 | AW897292 | | gb: CM0-NN0057-150400-338-b02 NN0057 Homo | 1 | 0.87 | upregulate stage |
| 455351 | AW901942 | | gb: QV0-NN1022-100400-190-b04 NN1022 Homo | 1 | 0.39 | upregulate stage |
| 455380 | BE160188 | | gb: QV1-HT0413-010200-059-g05 HT0413 Homo | 1.96 | 0.33 | upregulate stage |
| 455414 | AW936969 | | gb: RC1-DT0029-160200-013-f10 DT0029 Homo | 1 | 1 | upregulate stage |
| 455428 | AW938204 | | gb: QV0-DT0048-170200-124-f01 DT0048 Homo | 1 | 0.67 | upregulate stage |
| 455573 | BE004988 | | gb: MR2-BN0114-100500-020-b04 BN0114 Homo | 1 | 1 | upregulate stage |
| 455586 | BE070794 | | gb: RC3-BT0501-130100-011-h02 BT0501 Homo | 1 | 1 | upregulate stage |
| 455595 | BE008343 | | gb: CM0-BN0154-080400-325-g10 BN0154 Homo | 1 | 1 | upregulate stage |
| 455610 | BE011703 | | gb: CM3-BN0223-100500-177-h09 BN0223 Homo | 6.05 | 0.12 | upregulate stage |
| 455647 | BE064415 | | gb: RC4-BT0311-241199-012-b03 BT0311 Homo | 1 | 1 | upregulate stage |
| 455650 | BE064655 | | gb: RC1-BT0313-301299-012-c09 BT0313 Homo | 1 | 0.67 | upregulate stage |
| 455657 | BE065209 | | gb: RC1-BT0314-310300-015-b12 BT0314 Homo | 1.87 | 0.4 | upregulate stage |
| 455669 | BE065803 | | gb: RC2-BT0318-241199-011-g02 BT0318 Homo | 2.85 | 0.2 | upregulate stage |
| 455678 | BE066007 | | gb: RC3-BT0319-120200-014-d09 BT0319 Homo | 2.8 | 0.18 | upregulate stage |
| 455761 | BE080895 | | gb: QV1-BT0631-280200-084-e01 BT0631 Homo | 1 | 0.48 | upregulate stage |
| 455799 | BE169911 | Hs.14570 | Homo sapiens cDNA: FLJ22530 fis, clone H | 5.7 | 0.11 | upregulate stage |
| 455831 | BE144966 | | gb: RC6-HT0187-201099-031-c04 HT0187 Homo | 1 | 1 | upregulate stage |
| 455874 | BE152283 | | gb: QV4-HT0316-191199-039-b01 HT0316 Homo | 1 | 0.67 | upregulate stage |
| 455903 | BE155185 | | gb: PM1-HT0350-231299-005-g05 HT0350 Homo | 1 | 0.31 | upregulate stage |
| 455938 | BE159432 | | gb: MR0-HT0407-140200-009-e06 HT0407 Homo | 2.4 | 0.15 | upregulate stage |
| 455950 | BE161004 | | gb: PM0-HT0425-170100-002-h03 HT0425 Homo | 1 | 0.44 | upregulate stage |
| 455951 | BE161001 | | gb: PM0-HT0425-170100-002-f10 HT0425 Homo | 1 | 0.38 | upregulate stage |
| 455965 | BE167014 | | gb: CM2-HT0502-140200-088-d08 HT0502 Homo | 1 | 1 | upregulate stage |
| 455981 | BE177000 | | gb: RC4-HT0587-070400-015-b07 HT0587 Homo | 1 | 0.57 | upregulate stage |
| 456034 | AW450979 | | gb: UI-H-BI3-ala-a-12-0-UI.s1 NCI_CGAP_Su | 8.29 | 0.05 | upregulate stage |
| 456046 | R51494 | Hs.71818 | ESTs | 3.15 | 0.17 | upregulate stage |
| 456122 | R11813 | | gb: yf53a04.r1 Soares infant brain 1NIB H | 1.3 | 0.31 | upregulate stage |
| 456212 | N51636 | | gb: yy87b01.s1 Soares_multiple_sclerosis_{—} | 4.45 | 0.14 | upregulate stage |
| 456265 | AI968210 | Hs.173623 | ESTs | 1 | 0.34 | upregulate stage |
| 456285 | R67585 | Hs.268748 | ESTs | 1 | 0.83 | upregulate stage |
| 456320 | AI734064 | Hs.136212 | ESTs | 1 | 1 | upregulate stage |
| 456353 | AI042330 | Hs.87128 | ESTs, Weakly similar to similar to YBS4 | 5.15 | 0.11 | upregulate stage |
| 456486 | AA676544 | Hs.171545 | HIV-1 Rev binding protein | 1 | 0.27 | upregulate stage |
| 456493 | AA261830 | | gb: zs17g09.r1 NCI_CGAP_GCB1 Homo sapiens | 1 | 0.8 | upregulate stage |
| 456504 | AK000532 | Hs.98491 | Homo sapiens cDNA FLJ20525 fis, clone KA | 1 | 0.29 | upregulate stage |
| 456508 | AA502764 | Hs.123469 | ESTs, Weakly similar to AF208855 1 BM-01 | 17.7 | 0.05 | upregulate stage |
| 456519 | AA279917 | Hs.88678 | ESTs, Weakly similar to Unknown [H. sapie | 2.3 | 0.18 | upregulate stage |
| 456536 | AW135986 | Hs.257859 | ESTs | 9.45 | 0.06 | upregulate stage |
| 456592 | R91600 | | gb: yq10c02.r1 Soares fetal liver spleen | 4.5 | 0.14 | upregulate stage |
| 456621 | T35958 | Hs.107614 | DKFZP564I1171 protein | 1 | 0.2 | upregulate stage |
| 456682 | AW500321 | Hs.246766 | Homo sapiens cDNA FLJ12360 fis, clone MA | 1 | 0.24 | upregulate stage |
| 456726 | H43102 | Hs.144183 | ESTs | 1 | 0.69 | upregulate stage |
| 456736 | AW248217 | Hs.1619 | achaete-scute complex (Drosophila) homol | 0.89 | 0.91 | upregulate stage |
| 456786 | AK002084 | Hs.132851 | hypothetical protein FLJ11222 | 3.2 | 0.13 | upregulate stage |
| 456800 | AL118754 | | gb: DKFZp761P1910_r1 761 (synonym: hamy2) | 1 | 0.69 | upregulate stage |
| 456823 | AL161979 | Hs.146128 | Homo sapiens mRNA; cDNA DKFZp761G1823 (f | 8.95 | 0.07 | upregulate stage |
| 456844 | AI264155 | Hs.152981 | CDP-diacylglycerol synthase (phosphatida | 5.55 | 0.1 | upregulate stage |
| 456999 | AA319798 | Hs.172247 | eukaryotic translation elongation factor | 11.3 | 0.07 | upregulate stage |
| 457015 | AA688058 | Hs.261544 | ESTs | 9.25 | 0.08 | upregulate stage |
| 457030 | AI301740 | Hs.173381 | dihydropyrimidinase-like 2 | 2.65 | 0.17 | upregulate stage |
| 457158 | AA135370 | Hs.188536 | Homo sapiens cDNA: FLJ21635 fis, clone C | 1 | 1 | upregulate stage |
| 457190 | AI753247 | Hs.29643 | Homo sapiens cDNA FLJ13103 fis, clone NT | 1 | 0.87 | upregulate stage |
| 457309 | AF131843 | Hs.239340 | Homo sapiens clone 24987 mRNA sequence | 2.6 | 0.15 | upregulate stage |
| 457376 | AI026984 | Hs.293662 | ESTs | 1 | 1 | upregulate stage |
| 457402 | AW452648 | Hs.149342 | activation-induced cytidine deaminase | 2.9 | 0.16 | upregulate stage |
| 457435 | AW972024 | Hs.154645 | ESTs, Weakly similar to tyrosine kinase | 1 | 0.36 | upregulate stage |
| 457437 | AW969732 | | gb: EST381810 MAGE resequences, MAGK Homo | 2.5 | 0.14 | upregulate stage |
| 457465 | AW301344 | Hs.195969 | ESTs | 6.3 | 0.1 | upregulate stage |
| 457467 | AW974815 | Hs.292786 | ESTs | 1 | 1 | upregulate stage |
| 457474 | AW972935 | | gb: EST385031 MAGE resequences, MAGM Homo | 1 | 0.29 | upregulate stage |
| 457530 | AW973713 | Hs.293596 | ESTs | 1 | 0.39 | upregulate stage |
| 457637 | AI288373 | Hs.149875 | ESTs | 1 | 1 | upregulate stage |
| 457643 | AI375499 | Hs.27379 | ESTs | 3.25 | 0.19 | upregulate stage |
| 457650 | AA649162 | Hs.236456 | ESTs | 8.9 | 0.08 | upregulate stage |
| 457661 | AA917801 | Hs.128596 | ESTs | 0.96 | 0.9 | upregulate stage |
| 457692 | AA744046 | Hs.133350 | ESTs | 1 | 1 | upregulate stage |
| 457857 | AW814892 | Hs.273104 | ESTs | 1 | 1 | upregulate stage |
| 457892 | AA744389 | | gb: ny51e10.s1 NCI_CGAP_Pr18 Homo sapiens | 8.7 | 0.06 | upregulate stage |
| 457902 | AI624876 | Hs.75862 | MAD (mothers against decapentaplegic, Dr | 2.2 | 0.21 | upregulate stage |
| 457943 | AA765625 | Hs.155690 | ESTs | 3.55 | 0.1 | upregulate stage |
| 457948 | AI498640 | Hs.159354 | ESTs | 2.65 | 0.19 | upregulate stage |
| 457964 | NM_016353 | Hs.5943 | rec | 1.5 | 0.17 | upregulate stage |
| 458004 | AW976942 | Hs.153057 | ESTs | 1 | 0.87 | upregulate stage |
| 458027 | L49054 | Hs.85195 | ESTs, Highly similar to t(3; 5)(q25.1; p34 | 3.45 | 0.12 | upregulate stage |
| 458079 | AI796870 | Hs.54277 | ESTs | 11.5 | 0.05 | upregulate stage |
| 458158 | AW296778 | Hs.300357 | ESTs, Highly similar to dJ416F21.2 [H. sa | 1 | 1 | upregulate stage |
| 458171 | AI420016 | Hs.192090 | ESTs | 0.69 | 1.09 | upregulate stage |
| 458172 | BE007237 | | gb: PM0-BN0139-050500-003-g09 BN0139 Homo | 3 | 0.16 | upregulate stage |
| 458186 | AA904244 | Hs.153205 | ESTs | 4.6 | 0.15 | upregulate stage |
| 458242 | BE299588 | Hs.28465 | Homo sapiens cDNA: FLJ21869 fis, clone H | 3.1 | 0.16 | upregulate stage |
| 458270 | T66139 | Hs.113631 | ESTs | 1 | 0.67 | upregulate stage |
| 458282 | AA984075 | Hs.22580 | alkylglycerone phosphate synthase | 1 | 1 | upregulate stage |
| 458287 | AA987556 | Hs.12867 | ESTs | 5.05 | 0.13 | upregulate stage |
| 458580 | Z30118 | Hs.293788 | ESTs, Moderately similar to unnamed prot | 1 | 0.28 | upregulate stage |
| 458586 | AI683479 | Hs.65390 | ESTs | 8.2 | 0.07 | upregulate stage |
| 458608 | AW444662 | Hs.202247 | ESTs | 1 | 0.27 | upregulate stage |
| 458632 | AI744445 | Hs.24650 | Homo sapiens cDNA FLJ13047 fis, clone NT | 1.05 | 0.23 | upregulate stage |
| 458663 | AV658444 | Hs.280776 | Homo sapiens cDNA FLJ13684 fis, clone PL | 5.05 | 0.13 | upregulate stage |
| 458670 | AI301987 | Hs.233398 | ESTs | 8.9 | 0.08 | upregulate stage |
| 458680 | N73773 | Hs.282950 | ESTs | 1 | 0.23 | upregulate stage |
| 458720 | AV662037 | Hs.124740 | ESTs | 1 | 0.3 | upregulate stage |
| 458722 | AA741545 | Hs.282832 | ESTs | 3.2 | 0.11 | upregulate stage |
| 458747 | BE618395 | Hs.257391 | ESTs, Weakly similar to GTPase-activatin | 3.3 | 0.14 | upregulate stage |
| 458760 | AI498631 | Hs.111334 | ferritin, light polypeptide | 11 | 0.07 | upregulate stage |
| 458781 | AI444821 | | gb: RET4B7 sabtracted retina cDNA library | 6.05 | 0.12 | upregulate stage |
| 458801 | N98648 | Hs.276860 | ESTs | 4.45 | 0.13 | upregulate stage |
| 458880 | AA046742 | | gb: zf48c09.r1 Soares retina N2b4HR Homo | 9 | 0.08 | upregulate stage |
| 458886 | AI247487 | Hs.103277 | ESTs | 1 | 0.3 | upregulate stage |
| 458946 | AA009716 | Hs.42311 | ESTs | 8.7 | 0.08 | upregulate stage |
| 459023 | AW968226 | Hs.60798 | ESTs | 2.95 | 0.15 | upregulate stage |
| 459028 | AI940577 | | gb: IL5-HT0009-120799-001-G07 HT0009 Homo | 2.6 | 0.17 | upregulate stage |
| 459030 | H86658 | Hs.107699 | ESTs, Weakly similar to hypothetical pro | 1 | 1 | upregulate stage |
| 459058 | H85939 | Hs.209605 | ESTs | 1 | 1 | upregulate stage |
| 459128 | AI902169 | | gb: IL-BT002-221198-051 B1002 Homo sapien | 1 | 0.26 | upregulate stage |
| 459182 | BE178517 | | gb: PM1-HT0603-090300-001-e09 HT0603 Homo | 1 | 1 | upregulate stage |
| 459204 | AW194601 | Hs.13219 | ESTs | 2.85 | 0.16 | upregulate stage |
| 459256 | AW967468 | Hs.99821 | Homo sapiens mRNA; cDNA DKFZp564C046 (fr | 10.65 | 0.07 | upregulate stage |
| 459319 | NM_000059 | | gb: Homo sapiens breast cancer 2, early o | 1 | 1 | upregulate stage |
| 459395 | Z30300 | Hs.281935 | ESTs | 4.05 | 0.14 | upregulate stage |
| 459459 | AA460445 | | gb: zx66h11. r1 Soares_total_fetes_Nb2HF8_{—} | 4.8 | 0.13 | upregulate stage |
| 459464 | AA854847 | | gb: aj77h02.s1 Soares_parathyroid_tumor_N | 1 | 0.38 | upregulate stage |
| 459492 | AL118619 | | gb: DKFZp761E2410_r1 761 (synonym: hamy2) | 1 | 1 | upregulate stage |
| 459530 | AW770811 | | gb: hn49d07.x1 NCI_CGAP_Co17 Homo sapiens | 1 | 1 | upregulate stage |
| 401519 | | | | 12.65 | 0.06 | upregulate stage |
| 402474 | | | | 25.55 | 0.03 | upregulate stage |
| 402727 | | | | 16.25 | 0.05 | upregulate stage |
| 405411 | | | | 12.95 | 0.05 | upregulate stage |
| 406636 | L12064 | | gb: Homo sapiens (clone WR4.12VL) anti-th | 14.42 | 0.03 | upregulate stage |
| 406685 | M18728 | | gb: Human nonspecific crossreacting antig | 15.75 | 0.03 | upregulate stage |
| 407151 | H25836 | Hs.301527 | ESTs, Moderately similar to unknown [H.s | 16.3 | 0.04 | upregulate stage |
| 407242 | M18728 | | gb: Human nonspecific crossreacting antig | 12.56 | 0.03 | upregulate stage |
| 407347 | AA829847 | Hs.167347 | ESTs, Weakly similar to ALU8_HUMAN ALU S | 12.91 | 0.06 | upregulate stage |
| 407796 | AA195509 | Hs.272239 | lymphocyte activation-associated protein | 14.2 | 0.06 | upregulate stage |
| 408243 | Y00787 | Hs.624 | interleukin 8 | 18.52 | 0.02 | upregulate stage |
| 408380 | AF123050 | Hs.44532 | diubiquitin | 16 | 0.03 | upregulate stage |
| 408618 | AK000637 | Hs.46624 | HSPC043 protein | 12.6 | 0.06 | upregulate stage |
| 408741 | M73720 | Hs.646 | carboxypeptidase A3 (mast cell) | 15.5 | 0.03 | upregulate stage |
| 409417 | AA156247 | Hs.295908 | ESTs, Weakly similar to ALU7_HUMAN ALU S | 12.55 | 0.04 | upregulate stage |
| 410315 | AI638871 | Hs.17625 | ESTs | 14 | 0.05 | upregulate stage |
| 410324 | AW292539 | Hs.30177 | ESTs | 15.65 | 0.05 | upregulate stage |
| 412420 | AL035668 | Hs.73853 | bone morphogenetic protein 2 | 12.6 | 0.05 | upregulate stage |
| 412490 | AW803564 | Hs.288850 | Homo sapiens cDNA: FLJ22528 fis, clone H | 16.45 | 0.03 | upregulate stage |
| 413281 | AA861271 | Hs.34396 | ESTs | 12.95 | 0.04 | upregulate stage |
| 414004 | AA737033 | Hs.7155 | ESTs, Weakly similar to 2115357A TYKi pr | 15.25 | 0.04 | upregulate stage |
| 414161 | AA136106 | Hs.184852 | KIAA1553 protein | 13.25 | 0.06 | upregulate stage |
| 414217 | AI309298 | Hs.279898 | Homo sapiens cDNA: FLJ23165 fis, clone L | 12.5 | 0.05 | upregulate stage |
| 414219 | W20010 | Hs.75823 | ALL1-fused gene from chromosome 1q | 12.71 | 0.05 | upregulate stage |
| 414493 | AL133921 | Hs.76272 | retinoblastoma-binding protein 2 | 13.05 | 0.05 | upregulate stage |
| 414522 | AW518944 | Hs.76325 | Homo sapiens cDNA: FLJ23125 fis, clone L | 30.45 | 0.02 | upregulate stage |
| 414602 | AW630088 | Hs.76550 | Homo sapiens mRNA; cDNA DKFZp564B1264 (f | 29 | 0.02 | upregulate stage |
| 414761 | AU077228 | Hs.77256 | enhancer of zeste (Drosophila) homolog 2 | 13.2 | 0.05 | upregulate stage |
| 416114 | AI695549 | Hs.183868 | glucuronidase, beta | 14.7 | 0.04 | upregulate stage |
| 416179 | R19015 | Hs.79067 | MAD (mothers against decapentaplegic, Dr | 13 | 0.06 | upregulate stage |
| 416391 | AI878927 | Hs.79284 | mesoderm specific transcript (mouse) hom | 13.3 | 0.04 | upregulate stage |
| 416815 | U41514 | Hs.80120 | UDP-N-acetyl-alpha-D-galactosamine: polyp | 15.55 | 0.04 | upregulate stage |
| 416980 | AA381133 | Hs.80684 | high-mobility group (nonhistone chromoso | 23.85 | 0.03 | upregulate stage |
| 417258 | N58885 | Hs.294040 | ESTs | 15.05 | 0.06 | upregulate stage |
| 417274 | N92036 | Hs.81848 | RAD21 (S. pombe) homolog | 23.05 | 0.04 | upregulate stage |
| 417353 | AA375752 | Hs.76362 | general transcription factor IIA, 2 (12 k | 13 | 0.06 | upregulate stage |
| 417615 | BE548641 | Hs.82314 | hypoxanthine phosphoribosyltransferase 1 | 19.45 | 0.04 | upregulate stage |
| 417696 | BE241624 | Hs.82401 | CD69 antigen (p60, early T-cell activati | 12.45 | 0.03 | upregulate stage |
| 417777 | AI823763 | Hs.7055 | ESTs | 12.6 | 0.06 | upregulate stage |
| 417821 | BE245149 | Hs.82643 | protein tyrosine kinase 9 | 20.8 | 0.04 | upregulate stage |
| 417928 | AA209344 | Hs.282973 | ESTs | 14.65 | 0.05 | upregulate stage |
| 418699 | BE539639 | Hs.173030 | ESTs, Weakly similar to ALU8_HUMAN ALU S | 13 | 0.05 | upregulate stage |
| 418791 | AA935633 | Hs.194628 | ESTs | 12.95 | 0.06 | upregulate stage |
| 419145 | N99638 | | gb: za39g11.r1 Soares fetal liver spleen | 13.2 | 0.05 | upregulate stage |
| 421878 | AA299652 | Hs.111496 | Homo sapiens cDNA FLJ11643 fis, clone HE | 12.6 | 0.05 | upregulate stage |
| 422150 | AI867118 | Hs.2953 | ribosomal protein S15a | 13.55 | 0.05 | upregulate stage |
| 422363 | T55979 | Hs.115474 | replication factor C (activator 1) 3 (38 | 15.7 | 0.05 | upregulate stage |
| 424673 | AA345051 | Hs.294092 | ESTs | 16.9 | 0.04 | upregulate stage |
| 424848 | AI263231 | Hs.145607 | ESTs | 15.2 | 0.05 | upregulate stage |
| 424865 | AF011333 | Hs.153563 | lymphocyte antigen 75 | 12.85 | 0.04 | upregulate stage |
| 425053 | AF046024 | Hs.154320 | ubiquitin-activating enzyme E1C (homolog | 13.25 | 0.06 | upregulate stage |
| 425787 | AA363867 | Hs.155029 | ESTs | 17.55 | 0.05 | upregulate stage |
| 426252 | BE176980 | Hs.28917 | ESTs | 12.95 | 0,05 | upregulate stage |
| 426329 | AL389951 | Hs.271623 | nucleoporin 50 kD | 13.8 | 0.05 | upregulate stage |
| 427127 | AW802282 | Hs.22265 | pyruvate dehydrogenase phosphatase | 13.85 | 0.05 | upregulate stage |
| 427351 | AW402593 | Hs.123253 | Homo sapiens cDNA: FLJ22009 fis, clone H | 12.8 | 0.06 | upregulate stage |
| 427979 | BE379776 | Hs.181309 | proteasome (prosome, macropain) subunit, | 16.95 | 0.05 | upregulate stage |
| 428044 | AA093322 | Hs.182225 | RNA binding motif protein 3 | 14.65 | 0.05 | upregulate stage |
| 428428 | AL037544 | Hs.184298 | cyclin-dependent kinase 7 (homolog of Xe | 17.15 | 0.05 | upregulate stage |
| 428840 | M15990 | Hs.194148 | v-yes-1 Yamaguchi sarcoma viral oncogene | 16.8 | 0.05 | upregulate stage |
| 430191 | AI149880 | Hs.188809 | ESTs | 14.5 | 0.05 | upregulate stage |
| 430589 | AJ002744 | Hs.246315 | UDP-N-acetyl-alpha-D-galactosamine: polyp | 14.9 | 0.05 | upregulate stage |
| 430853 | AI734179 | Hs.105676 | ESTs | 13.55 | 0.06 | upregulate stage |
| 431049 | AA846576 | Hs.103267 | hypothetical protein FLJ22548 similar to | 16.2 | 0.04 | upregulate stage |
| 431211 | M86849 | Hs.5566 | gap junction protein, beta 2, 26 kD (conn | 27 | 0.01 | upregulate stage |
| 431341 | AA307211 | Hs.251531 | proteasome (prosome, macropain) subunit, | 13.45 | 0.06 | upregulate stage |
| 431639 | AK000680 | Hs.266175 | phosphoprotein associated with GEMs | 21.2 | 0.03 | upregulate stage |
| 431770 | BE221880 | Hs.268555 | 5′-3′ exoribonuclease 2 | 13.05 | 0.06 | upregulate stage |
| 431863 | AA188185 | Hs.271871 | spindlin | 15.6 | 0.05 | upregulate stage |
| 434263 | N34895 | Hs.44648 | ESTs | 14.25 | 0.05 | upregulate stage |
| 434651 | BE616902 | Hs.285313 | core promoter element binding protein | 17.95 | 0.05 | upregulate stage |
| 436286 | AA804442 | Hs.3459 | Homo sapiens cDNA: FLJ22003 fis, clone H | 14.95 | 0.05 | upregulate stage |
| 436385 | BE551618 | Hs.144097 | ESTs | 13.85 | 0.06 | upregulate stage |
| 437192 | AW975786 | Hs.75355 | ubiquitin-conjugating enzyme E2N (homolo | 12.75 | 0.06 | upregulate stage |
| 438000 | AI825880 | Hs.5985 | non-kinase Cdc42 effector protein SPEC2 | 15.3 | 0.04 | upregulate stage |
| 439941 | AI392640 | Hs.18272 | ESTs | 17.42 | 0.05 | upregulate stage |
| 440086 | NM_005402 | Hs.288757 | v-ral simian leukemia viral oncogene hom | 12.59 | 0.05 | upregulate stage |
| 440116 | AI798851 | Hs.9403 | ESTs | 14.5 | 0.05 | upregulate stage |
| 441020 | W79283 | Hs.35962 | ESTs | 12.4 | 0.04 | upregulate stage |
| 441633 | AW958544 | Hs.112242 | ESTs | 15.85 | 0.03 | upregulate stage |
| 441980 | AK001441 | Hs.8055 | hypothetical protein FLJ10579 | 13.6 | 0.05 | upregulate stage |
| 442043 | BE567620 | Hs.99210 | ESTs | 12.5 | 0.06 | upregulate stage |
| 442053 | R35343 | Hs.24968 | Human DNA sequence from clone RP1-233G16 | 12.65 | 0.06 | upregulate stage |
| 442271 | AF000652 | Hs.8180 | syndecan binding protein (syntenin) | 15.15 | 0.05 | upregulate stage |
| 443303 | U67319 | Hs.9216 | caspase 7, apoptosis-related cysteine pr | 13.4 | 0.05 | upregulate stage |
| 445033 | AV652402 | Hs.155145 | ESTs | 13.3 | 0.05 | upregulate stage |
| 446619 | AU076643 | Hs.313 | secreted phosphoprotein 1 (osteopontin, | 30.5 | 0.02 | upregulate stage |
| 446847 | T51454 | Hs.82845 | Human clone 23815 mRNA sequence | 13.8 | 0.04 | upregulate stage |
| 446921 | AB012113 | Hs.16530 | small inducible cytokine subfamily A (Cy | 15.15 | 0.04 | upregulate stage |
| 448712 | W01046 | Hs.181634 | Homo sapiens cDNA: FLJ23602 fis, clone L | 13.2 | 0.05 | upregulate stage |
| 448772 | AW390822 | Hs.24639 | ESTs | 12.75 | 0.06 | upregulate stage |
| 448926 | AI798164 | Hs.140903 | ESTs, Moderately similar to neuronal thr | 13.35 | 0.06 | upregulate stage |
| 449962 | AA004879 | No.187820 | ESTs | 12.79 | 0.05 | upregulate stage |
| 450139 | AK001838 | Hs.296323 | Homo sapiens cDNA FLJ10976 fis, clone PL | 14.76 | 0.06 | upregulate stage |
| 451035 | AU076785 | Hs.430 | plastin 1 (I isoform) | 17.65 | 0.04 | upregulate stage |
| 451334 | AI122691 | Hs.13268 | ESTs | 14.7 | 0.05 | upregulate stage |
| 452567 | D87120 | Hs.29882 | predicted osteoblast protein | 12.45 | 0.06 | upregulate stage |
| 453258 | AW293134 | Hs.32597 | ring finger protein (C3H2C3 type) 6 | 13.4 | 0.05 | upregulate stage |
| 453331 | AI240665 | Hs.8895 | ESTs | 12.6 | 0.05 | upregulate stage |
| 400365 | Y10259 | Hs.274501 | H. sapiens ACTH receptor mRNA 3′UTR | 2.2 | 0.17 | upregulate stage |
| 401256 | | | | 2 | 0.16 | upregulate stage |
| 402075 | | | | 1 | 0.1 | upregulate stage |
| 403029 | | | | 1.75 | 0.16 | upregulate stage |
| 403047 | | | | 3.3 | 0.1 | upregulate stage |
| 403426 | | | | 1.7 | 0.18 | upregulate stage |
| 403754 | | | | 2.8 | 0.12 | upregulate stage |
| 403822 | | | | 1.2 | 0.14 | upregulate stage |
| 407835 | AK002081 | Hs.40337 | hypothetical protein | 1.9 | 0.15 | upregulate stage |
| 407980 | AA046309 | | gb: zf12f01.s1 Soares_fetal_heart_NbHH19W | 1.35 | 0.1 | upregulate stage |
| 408081 | AW451597 | Hs.167409 | ESTs | 2.3 | 0.18 | upregulate stage |
| 408408 | AF070571 | Hs.44690 | Homo sapiens clone 24739 mRNA sequence | 1.65 | 0.12 | upregulate stage |
| 408920 | AL120071 | Hs.48998 | fibronectin leucine rich transmembrane p | 1 | 0.2 | upregulate stage |
| 409810 | AW500895 | | gb: UI-HF-BP0p-air-a-02-0-UI.r1 NIH_MGC_5 | 2.25 | 0.2 | upregulate stage |
| 410094 | BE147897 | Hs.58593 | general transcription factor IIF, polype | 4.05 | 0.12 | upregulate stage |
| 410603 | AA086219 | Hs.68714 | ESTs | 1.9 | 0.18 | upregulate stage |
| 410763 | AF279145 | Hs.8966 | tumor endothelial marker 8 | 4.15 | 0.13 | upregulate stage |
| 411418 | BE241870 | | gb: TCAAP2E0047 Pediatric acute myelogeno | 1.6 | 0.22 | upregulate stage |
| 411691 | AW857199 | | gb: RC2-CT0304-080100-011-f06 CT0304 Homo | 1.45 | 0.24 | upregulate stage |
| 411750 | BE562298 | Hs.71827 | KIAA0112 protein; homolog of yeast ribos | 2.2 | 0.2 | upregulate stage |
| 411880 | AW872477 | | gb: hm30f03.x1 NCI_CGAP_Thy4 Homo sapiens | 1.75 | 0.06 | upregulate stage |
| 412102 | H56435 | Hs.75935 | KIAA0077 protein | 1.7 | 0.2 | upregulate stage |
| 412303 | AW936336 | | gb: QV4-DT0021-281299-070-g11 DT0021 Homo | 1 | 0.17 | upregulate stage |
| 412312 | AW936686 | | gb: PM2-DT0023-080300-004-g01 DT0023 Homo | 3.4 | 0.16 | upregulate stage |
| 412598 | AI681997 | Hs.107057 | ESTs | 2.25 | 0.2 | upregulate stage |
| 413383 | AI128978 | Hs.154706 | Homo sapiens cDNA FLJ13594 fis, clone PL | 2.3 | 0.17 | upregulate stage |
| 413406 | AW452823 | Hs.135268 | ESTs | 3.52 | 0.14 | upregulate stage |
| 413618 | BE154078 | | gb: PM0-HT0339-200400-010-F04 HT0339 Homo | 1 | 0.18 | upregulate stage |
| 416661 | AA634543 | Hs.79440 | IGF-II mRNA-binding protein 3 | 1.05 | 0.12 | upregulate stage |
| 417708 | N74392 | Hs.50495 | ESTs | 2 | 0.16 | upregulate stage |
| 417974 | AA210765 | | gb: zr50c06.r1 NCI_CGAP_GCB1 Homo sapiens | 1.7 | 0.18 | upregulate stage |
| 418604 | AA225632 | Hs.190016 | ESTs | 3.75 | 0.13 | upregulate stage |
| 418631 | AA225921 | Hs.115105 | ESTs | 1.75 | 0.2 | upregulate stage |
| 418830 | BE513731 | Hs.88959 | Human DNA sequence from clone 967N21 on | 3.8 | 0.09 | upregulate stage |
| 418893 | N32264 | Hs.44330 | ESTs | 2.35 | 0.14 | upregulate stage |
| 418950 | T78517 | Hs.13941 | ESTs | 2.15 | 0.19 | upregulate stage |
| 419044 | AI799135 | Hs.87164 | Homo sapiens cDNA FLJ14001 fis, clone Y7 | 1.85 | 0.15 | upregulate stage |
| 420082 | N43741 | Hs.203148 | ESTs | 3.8 | 0.14 | upregulate stage |
| 420653 | AI224532 | Hs.88550 | ESTs | 2.05 | 0.16 | upregulate stage |
| 421112 | AW243875 | Hs.265427 | ESTs | 3.3 | 0.13 | upregulate stage |
| 421683 | AI147535 | Hs.143769 | ESTs | 2 | 0.14 | upregulate stage |
| 421799 | AW972292 | Hs.292998 | ESTs | 2.35 | 0.15 | upregulate stage |
| 422177 | AA720878 | Hs.201375 | ESTs | 3.3 | 0.14 | upregulate stage |
| 422429 | AA310527 | | gb: EST181333 Jurkat T-cells V Home sapie | 3.45 | 0.12 | upregulate stage |
| 422956 | BE545072 | Hs.122579 | hypothetical protein FLJ10461 | 2.15 | 0.11 | upregulate stage |
| 424026 | AI798295 | Hs.123218 | ESTs | 3.8 | 0.14 | upregulate stage |
| 425650 | NM_001944 | Hs.1925 | desmoglein 3 (pemphigus vulgaris antigen | 1 | 0.09 | upregulate stage |
| 425761 | AW664214 | Hs.196729 | ESTs | 2 | 0.19 | upregulate stage |
| 426427 | M86699 | Hs.169840 | TTK protein kinase | 2.1 | 0.16 | upregulate stage |
| 427558 | D49493 | Hs.2171 | growth differentiation factor 10 | 2.15 | 0.14 | upregulate stage |
| 427635 | BE397988 | Hs.179982 | tumor protein p53-binding protein | 3.9 | 0.11 | upregulate stage |
| 428766 | AA477989 | Hs.98800 | ESTs | 3.8 | 0.12 | upregulate stage |
| 429761 | AI276780 | Hs.135173 | ESTs | 1.9 | 0.17 | upregulate stage |
| 430132 | AA204686 | Hs.234149 | hypothetical protein FLJ20647 | 5.05 | 0.11 | upregulate stage |
| 430253 | AK001514 | Hs.236844 | hypothetical protein FLJ10652 | 3.55 | 0.15 | upregulate stage |
| 430388 | AA356923 | Hs.240770 | nuclear cap binding protein subunit 2, 2 | 2.5 | 0.14 | upregulate stage |
| 431187 | AW971146 | Hs.293187 | ESTs | 3.95 | 0.13 | upregulate stage |
| 431364 | AW971382 | Hs.294016 | ESTs, Weakly similar to alpha-1 (XVIII) c | 1.8 | 0.15 | upregulate stage |
| 431401 | AA504626 | Hs.105735 | ESTs | 1.65 | 0.22 | upregulate stage |
| 431419 | AL041844 | Hs.277522 | ESTs, Weakly similar to FYVE finger-cont | 1.45 | 0.16 | upregulate stage |
| 432361 | AI378562 | Hs.159585 | ESTs | 2.15 | 0.14 | upregulate stage |
| 432810 | AA863400 | Hs.23054 | ESTs | 3.7 | 0.08 | upregulate stage |
| 432926 | AA570416 | Hs.32271 | hypothetical protein FLJ10846 | 2 | 0.2 | upregulate stage |
| 433108 | AB002446 | | gb: Homo sapiens mRNA from chromosome 5q2 | 2.35 | 0.14 | upregulate stage |
| 434153 | AF118072 | Hs.283916 | Homo sapiens PRO1716 mRNA, complete cds | 1 | 0.14 | upregulate stage |
| 435202 | AI971313 | Hs.170204 | KIAA0551 protein | 1.25 | 0.16 | upregulate stage |
| 435313 | AI769400 | Hs.189729 | ESTs | 2 | 0.18 | upregulate stage |
| 435359 | T60843 | Hs.189679 | ESTs | 3.6 | 0.11 | upregulate stage |
| 435488 | H57954 | Hs.34394 | ESTs | 2.2 | 0.22 | upregulate stage |
| 436583 | AW293909 | Hs.156935 | ESTs | 1.4 | 0.19 | upregulate stage |
| 436862 | AI821940 | Hs.264622 | ESTs, Moderately similar to ALU8_HUMAN A | 3.2 | 0.12 | upregulate stage |
| 437485 | AI149570 | Hs.127363 | ESTs | 2.05 | 0.22 | upregulate stage |
| 437854 | AL119723 | | gb: DKFZp761A2124_r1 761 (synonym: hamy2) | 2.75 | 0.15 | upregulate stage |
| 438316 | AA789249 | | gb: aj27g08.s1 Soares_testis_NHT Homo sap | 2.45 | 0.13 | upregulate stage |
| 438390 | AI422017 | | gb: tt45f12.x1 NCI_CGAP_Brn23 Homo sapien | 3.1 | 0.13 | upregulate stage |
| 438915 | AA280174 | Hs.23282 | ESTs | 1.35 | 0.12 | upregulate stage |
| 439983 | AA858394 | Hs.117955 | ESTs | 4 | 0.13 | upregulate stage |
| 442048 | AA974603 | | gb: op34f05.s1 Soares_NFL_T_GBC_S1 Homo s | 5.55 | 0.09 | upregulate stage |
| 442369 | AI565071 | Hs.159983 | ESTs | 3.85 | 0.14 | upregulate stage |
| 442748 | AI016713 | Hs.135787 | ESTs | 2.35 | 0.23 | upregulate stage |
| 443717 | BE163884 | Hs.282331 | ESTs | 2.5 | 0.18 | upregulate stage |
| 445935 | AA287537 | Hs.167585 | ESTs | 1 | 0.2 | upregulate stage |
| 446078 | AI339982 | Hs.156061 | ESTs | 2.25 | 0.24 | upregulate stage |
| 446139 | H77395 | Hs.39749 | ESTs | 2.15 | 0.18 | upregulate stage |
| 446183 | AA354991 | Hs.14222 | Homo sapiens mRNA; cDNA DKFZp761P019 (fr | 3.45 | 0.14 | upregulate stage |
| 448253 | H25899 | Hs.201591 | ESTs | 1.65 | 0.18 | upregulate stage |
| 448956 | AK001674 | Hs.22630 | cofactor required for Sp1 transcriptiona | 2.2 | 0.14 | upregulate stage |
| 449199 | AI990122 | Hs.196988 | ESTs | 1.25 | 0.23 | upregulate stage |
| 449558 | AA001765 | Hs.157079 | KIAA1227 protein | 1 | 0.16 | upregulate stage |
| 449576 | AW014631 | Hs.225068 | ESTs | 2.3 | 0.19 | upregulate stage |
| 449859 | T98077 | Hs.18214 | ESTs | 6.3 | 0.07 | upregulate stage |
| 450434 | AA166950 | Hs.18645 | ESTs, Weakly similar to partial CDS [C.e | 1.65 | 0.22 | upregulate stage |
| 450625 | AW970107 | | gb: EST382188 MAGE resequences, MAGK Homo | 1.35 | 0.19 | upregulate stage |
| 451337 | AI400209 | Hs.60787 | ESTs | 1.6 | 0.16 | upregulate stage |
| 451686 | AA059246 | Hs.110293 | ESTs | 3.4 | 0.14 | upregulate stage |
| 452079 | AA830908 | Hs.15825 | ESTs | 1.9 | 0.23 | upregulate stage |
| 452220 | BE158006 | Hs.212296 | ESTs | 3.1 | 0.17 | upregulate stage |
| 453918 | AW005123 | Hs.231975 | ESTs | 1 | 0.21 | upregulate stage |
| 455350 | AW901809 | | gb: QV0-NN1020-170400-195-h02 NN1020 Homo | 2 | 0.2 | upregulate stage |
| 456511 | AA282330 | Hs.145668 | ESTs | 1.15 | 0.12 | upregulate stage |
| 456986 | D38299 | Hs.170917 | prostaglandin E receptor 3 (subtype EP3) | 1.65 | 0.18 | upregulate stage |
| 457427 | AW971287 | | gb: EST383376 MAGE resequences, MAGL Homo | 2.35 | 0.16 | upregulate stage |
| 400296 | AA305627 | Hs.139336 | ATP-binding cassette, sub-family C (CFTR | 1 | 0.27 | upregulate stage |
| 400409 | AF153341 | Hs.283954 | Homo sapiens winged helix/forkhead trans | 2.33 | 0.2 | upregulate stage |
| 400471 | | | | 7.45 | 0.09 | upregulate stage |
| 400641 | | | | 0.71 | 0.31 | upregulate stage |
| 400749 | | | | 7.25 | 0.1 | upregulate stage |
| 400751 | | | | 5.35 | 0.09 | upregulate stage |
| 400761 | | | | 5.9 | 0.1 | upregulate stage |
| 400843 | | | | 5.85 | 0.07 | upregulate stage |
| 401045 | | | | 2.42 | 0.17 | upregulate stage |
| 401049 | | | | 1.2 | 0.19 | upregulate stage |
| 401192 | | | | 2.47 | 0.3 | upregulate stage |
| 401203 | | | | 6.73 | 0.08 | upregulate stage |
| 401205 | | | | 6.63 | 0.1 | upregulate stage |
| 401276 | | | | 6.95 | 0.1 | upregulate stage |
| 401561 | | | | 2.2 | 0.13 | upregulate stage |
| 401604 | | | | 1 | 0.19 | upregulate stage |
| 402245 | | | | 7.65 | 0.09 | upregulate stage |
| 402296 | | | | 1 | 0.33 | upregulate stage |
| 402530 | | | | 5.1 | 0.13 | upregulate stage |
| 402812 | | | | 1.65 | 0.17 | upregulate stage |
| 402820 | | | | 1 | 0.34 | upregulate stage |
| 402892 | | | | 1 | 1 | upregulate stage |
| 403344 | | | | 6.5 | 0.08 | upregulate stage |
| 404156 | | | | 3.7 | 0.11 | upregulate stage |
| 404290 | | | | 4.45 | 0.09 | upregulate stage |
| 404538 | | | | 8.38 | 0.09 | upregulate stage |
| 404676 | | | | 8.3 | 0.09 | upregulate stage |
| 404977 | | | | 0.9 | 0.35 | upregulate stage |
| 405033 | | | | 1.52 | 0.31 | upregulate stage |
| 405109 | N47812 | Hs.81360 | CGI-35 protein | 6.2 | 0.1 | upregulate stage |
| 405654 | | | | 1.95 | 0.06 | upregulate stage |
| 406081 | | | | 3 | 0.07 | upregulate stage |
| 406270 | | | | 6.09 | 0.13 | upregulate stage |
| 406399 | | | | 1.55 | 0.41 | upregulate stage |
| 406475 | | | | 6.2 | 0.12 | upregulate stage |
| 406485 | | | | 1 | 0.48 | upregulate stage |
| 406741 | AA058357 | Hs.74466 | carcinoembryonic antigen-related cell ad | 5.4 | 0.07 | upregulate stage |
| 406867 | AA157857 | Hs.182265 | keratin 19 | 2.26 | 0.37 | upregulate stage |
| 407173 | T64349 | | gb: yc10d08.s1 Stratagene lung (937210) H | 3.35 | 0.11 | upregulate stage |
| 407230 | AA157857 | Hs.182265 | keratin 19 | 2.15 | 0.38 | upregulate stage |
| 407266 | AJ235664 | | gb: Homo sapiens mRNA for immunoglobulin | 2.1 | 0.09 | upregulate stage |
| 407783 | AW996872 | Hs.172028 | a disintegrin and metalloproteinase doma | 3.25 | 0.11 | upregulate stage |
| 407825 | NM_006152 | Hs.40202 | lymphoid-restricted membrane protein | 6.25 | 0.08 | upregulate stage |
| 407870 | AB032990 | Hs.40719 | hypothetical protein KIAA1164 | 4.5 | 0.12 | upregulate stage |
| 407877 | AW016811 | Hs.234478 | Homo sapiens cDNA: FLJ22648 fis, clone H | 3.3 | 0.15 | upregulate stage |
| 407968 | NM_004863 | Hs.59403 | serine palmitoyltransferase, long chain | 7.35 | 0.1 | upregulate stage |
| 408162 | AA993833 | Hs.118527 | ESTs | 6.2 | 0.09 | upregulate stage |
| 408363 | NM_003389 | Hs.44396 | coronin, actin-binding protein, 2A | 5.36 | 0.14 | upregulate stage |
| 408576 | NM_003542 | Hs.46423 | H4 histone family, member G | 7.28 | 0.1 | upregulate stage |
| 408673 | BE208517 | Hs.184109 | ribosomal protein L37a | 2.53 | 0.24 | upregulate stage |
| 408684 | R61377 | Hs.12727 | hypothetical protein FLJ21610 | 1 | 0.3 | upregulate stage |
| 409361 | NM_005982 | Hs.54416 | sine oculis homeobox (Drosophila) homolo | 7.7 | 0.06 | upregulate stage |
| 409592 | BE280951 | Hs.55058 | EH-domain containing 4 | 3.95 | 0.1 | upregulate stage |
| 409744 | AW675258 | Hs.56265 | Homo sapiens mRNA; cDNA DKFZp586P2321 (f | 1.55 | 0.16 | upregulate stage |
| 410141 | R07775 | Hs.287657 | Homo sapiens cDNA: FLJ21291 fis, clone C | 4.1 | 0.18 | upregulate stage |
| 410232 | AW372451 | Hs.61184 | CGI-79 protein | 3.65 | 0.14 | upregulate stage |
| 410269 | AW613597 | | gb: hh79g12.x1 NCI_CGAP_GU1 Homo sapiens | 7.55 | 0.09 | upregulate stage |
| 410297 | AA148710 | Hs.159441 | ESTs | 3.8 | 0.1 | upregulate stage |
| 410337 | M83822 | Hs.62354 | cell division cycle 4-like | 4.35 | 0.19 | upregulate stage |
| 410418 | D31382 | Hs.63325 | transmembrane protease, serine 4 | 1.42 | 0.4 | upregulate stage |
| 410541 | AA065003 | Hs.64179 | hypothetical protein | 1.61 | 0.48 | upregulate stage |
| 410724 | AW799269 | | gb: RC0-UM0051-210300-012-f01 UM0051 Homo | 6.65 | 0.12 | upregulate stage |
| 410785 | AW803341 | | gb: IL2-UM0079-090300-050-D03 UM0079 Homo | 1.4 | 0.16 | upregulate stage |
| 410968 | AA199907 | Hs.67397 | homeo box A1 | 3.05 | 0.1 | upregulate stage |
| 411162 | AW819944 | | gb: QV0-ST0294-240300-172-e03 ST0294 Homo | 2 | 0.23 | upregulate stage |
| 411173 | R81571 | | gb: yj02h10.r1 Soares placenta Nb2HP Homo | 7.2 | 0.1 | upregulate stage |
| 411243 | AB039886 | Hs.69319 | CA11 | 0.36 | 0.93 | upregulate stage |
| 411407 | R00903 | | gb: ye87a07.r1 Soares fetal liver spleen | 8 | 0.09 | upregulate stage |
| 411704 | AI499220 | Hs.71573 | hypothetical protein FLJ10074 | 1.75 | 0.22 | upregulate stage |
| 412121 | AB033061 | Hs.73287 | KIAA1235 protein | 5.3 | 0.11 | upregulate stage |
| 412123 | BE251328 | Hs.73291 | hypothetical protein FLJ10881 | 6.9 | 0.1 | upregulate stage |
| 412129 | M21984 | Hs.73454 | troponin T3, skeletal, fast | 0.27 | 1.06 | upregulate stage |
| 412354 | AW939148 | | gb: QV1-DT0069-110200-067-d06 DT0069 Homo | 6.9 | 0.11 | upregulate stage |
| 412610 | X90908 | Hs.74126 | fatty acid binding protein 6, ileal (gas | 2.88 | 0.21 | upregulate stage |
| 412700 | BE222433 | Hs.201262 | ESTs | 2.85 | 0.15 | upregulate stage |
| 412706 | R97106 | Hs.167546 | ESTs | 3.75 | 0.16 | upregulate stage |
| 412935 | BE267045 | Hs.75064 | tubulin-specific chaperone c | 7 | 0.09 | upregulate stage |
| 413402 | T24065 | | gb: seq2245 HMSWMYK Homo sapiens cDNA clo | 6.3 | 0.12 | upregulate stage |
| 413431 | AW246428 | Hs.75355 | ubiquitin-conjugating enzyme E2N (homolo | 3.45 | 0.11 | upregulate stage |
| 413445 | BE141022 | | gb: MR0-HT0067-201099-002-d10 HT0067 Homo | 3.9 | 0.13 | upregulate stage |
| 413587 | AA156164 | Hs.271833 | Homo sapiens cDNA FLJ13473 fis, clone PL | 7.63 | 0.09 | upregulate stage |
| 413800 | AI129238 | Hs.192235 | ESTs | 3.2 | 0.18 | upregulate stage |
| 413859 | AW992356 | Hs.8364 | pyruvate dehydrogenase kinase, isoenzyme | 2.54 | 0.33 | upregulate stage |
| 413930 | M86153 | Hs.75618 | RAB11A, member RAS oncogene family | 1.75 | 0.21 | upregulate stage |
| 413991 | H44725 | Hs.71300 | ESTs | 1.3 | 0.21 | upregulate stage |
| 414052 | AW578849 | Hs.283552 | ESTs, Weakly similar to unnamed protein | 8.1 | 0.07 | upregulate stage |
| 414203 | BE262170 | | gb: 601150419F1 NIH_MGC_19 Homo sapiens c | 1.45 | 0.14 | upregulate stage |
| 414343 | AL036166 | Hs.75914 | coated vesicle membrane protein | 1 | 0.23 | upregulate stage |
| 414664 | AA587775 | Hs.66295 | Homo sapiens HSPC311 mRNA, partial cds | 1 | 0.36 | upregulate stage |
| 414987 | AA524394 | Hs.165544 | ESTs | 1.51 | 0.51 | upregulate stage |
| 414993 | AW819403 | Hs.77724 | KIAA0586 gene product | 2.72 | 0.23 | upregulate stage |
| 415276 | U88666 | Hs.78353 | SFRS protein kinase 2 | 6.95 | 0.1 | upregulate stage |
| 415303 | R11813 | | gb: yf53a04.r1 Soares infant brain 1NIB H | 8.1 | 0.09 | upregulate stage |
| 415392 | Z44067 | | gb: HSC1RF051 normalized infant brain cDN | 5.56 | 0.11 | upregulate stage |
| 415572 | F12294 | | gb: HSC38B051 normalized infant brain cDN | 5.7 | 0.11 | upregulate stage |
| 415773 | R21651 | | gb: yh19g02.r1 Soares placenta Nb2HP Homo | 5.3 | 0.11 | upregulate stage |
| 416012 | AF061959 | Hs.78961 | protein phosphatase 1, regulatory (inhib | 2.19 | 0.28 | upregulate stage |
| 416074 | R40174 | Hs.21209 | ESTs | 7.61 | 0.11 | upregulate stage |
| 416182 | NM_004354 | Hs.79069 | cyclin G2 | 1 | 0.39 | upregulate stage |
| 416518 | H60730 | Hs.18917 | ESTs | 6.6 | 0.1 | upregulate stage |
| 416782 | L35035 | Hs.79886 | ribose 5-phosphate isomerase A (ribose 5 | 3.9 | 0.17 | upregulate stage |
| 416987 | D86957 | Hs.80712 | KIAA0202 protein | 2.54 | 0.31 | upregulate stage |
| 417094 | NM_006895 | Hs.81182 | histamine N-methyltransferase | 4.26 | 0.12 | upregulate stage |
| 417275 | X63578 | Hs.81849 | parvalbumin | 1 | 0.12 | upregulate stage |
| 417395 | BE564245 | Hs.82084 | integrin beta 3 binding protein (beta3-e | 8.4 | 0.08 | upregulate stage |
| 417683 | AW566008 | Hs.239154 | Homo sapiens cDNA FLJ12814 fis, clone NT | 2.2 | 0.17 | upregulate stage |
| 417759 | R13567 | Hs.12548 | ESTs | 8.18 | 0.09 | upregulate stage |
| 417848 | AA206581 | Hs.39457 | ESTs | 8.6 | 0.08 | upregulate stage |
| 417985 | AA187545 | Hs.83114 | crystallin, zeta (quinone reductase) | 7 | 0.11 | upregulate stage |
| 418073 | R39789 | Hs.119714 | EST | 6.3 | 0.11 | upregulate stage |
| 418394 | AF132818 | Hs.84728 | Kruppel-like factor 5 (intestinal) | 1.63 | 0.46 | upregulate stage |
| 418406 | X73501 | Hs.84905 | cytokeratin 20 | 3.5 | 0.02 | upregulate stage |
| 418555 | AI417215 | Hs.87159 | Homo sapiens cDNA FLJ12577 fis, clone NT | 6.75 | 0.06 | upregulate stage |
| 418636 | AW749855 | | gb: QV4-BT0534-281299-053-c05 BT0534 Homo | 4.1 | 0.11 | upregulate stage |
| 418786 | AI796317 | Hs.203594 | Homo sapiens uncharacterized gastric pro | 7.5 | 0.08 | upregulate stage |
| 418827 | BE327311 | Hs.47166 | HT021 | 5.6 | 0.13 | upregulate stage |
| 418948 | AI217097 | | gb: qd43h07.x1 Soares_fetal_heart_NbHH19W | 1.5 | 0.22 | upregulate stage |
| 419551 | AW582256 | Hs.91011 | anterior gradient 2 (Xenepus laevis) hom | 2.44 | 0.29 | upregulate stage |
| 419590 | AF005043 | Hs.91390 | poly (ADP-ribose) glycohydrolase | 8.08 | 0.1 | upregulate stage |
| 419693 | AA133749 | Hs.92323 | FXYD domain-containing ion transport reg | 1.64 | 0.48 | upregulate stage |
| 419712 | AA360838 | Hs.179909 | Homo sapiens cDNA: FLJ22995 fis, clone K | 5.4 | 0.11 | upregulate stage |
| 419713 | AW968058 | Hs.92381 | nudix (nucleoside diphosphate linked moi | 7.9 | 0.06 | upregulate stage |
| 419720 | AA249131 | Hs.143607 | hypothetical protein FLJ11068 | 2.95 | 0.15 | upregulate stage |
| 419791 | AI579909 | Hs.105104 | ESTs | 2.45 | 0.2 | upregulate stage |
| 419872 | AI422951 | Hs.146162 | ESTs | 4.25 | 0.17 | upregulate stage |
| 419903 | T16938 | Hs.87902 | ESTs | 2.5 | 0.22 | upregulate stage |
| 419932 | AA281594 | | gb: zt03a01.r1 NCI_CGAP_GCB1 Homo sapiens | 6.1 | 0.12 | upregulate stage |
| 420026 | AI831190 | Hs.166676 | ESTs | 3.4 | 0.14 | upregulate stage |
| 420187 | AK001714 | Hs.95744 | hypothetical protein similar to ankyrin | 4.03 | 0.18 | upregulate stage |
| 420193 | AI460080 | Hs.202869 | ESTs | 1 | 0.28 | upregulate stage |
| 420281 | AI623693 | Hs.191533 | ESTs | 6.6 | 0.11 | upregulate stage |
| 420337 | AW295840 | Hs.14555 | Homo sapiens cDNA: FLJ21513 fis, clone C | 6 | 0.09 | upregulate stage |
| 420370 | Y13645 | Hs.97234 | uroplakin 2 | 1.2 | 0.45 | upregulate stage |
| 420383 | T55154 | Hs.144880 | ESTs | 3.8 | 0.12 | upregulate stage |
| 420450 | AW968969 | Hs.177726 | ESTs | 2.75 | 0.14 | upregulate stage |
| 420588 | AF000982 | Hs.147916 | DEAD/H (Asp-Glu-Ala-Asp/His) box polypep | 8.1 | 0.09 | upregulate stage |
| 420763 | AA419392 | Hs.178354 | ESTs | 8 | 0.09 | upregulate stage |
| 420838 | AW118210 | Hs.5244 | ESTs | 8.65 | 0.07 | upregulate stage |
| 420981 | L40904 | Hs.100724 | peroxisome proliferative activated recep | 1.98 | 0.32 | upregulate stage |
| 421013 | M62397 | Hs.1345 | mutated in colorectal cancers | 1 | 0.29 | upregulate stage |
| 421072 | AI215069 | Hs.89113 | ESTs | 5.8 | 0.12 | upregulate stage |
| 421110 | AJ250717 | Hs.1355 | cathepsin E | 5.45 | 0.03 | upregulate stage |
| 421141 | AW117261 | Hs.125914 | ESTs | 2.75 | 0.16 | upregulate stage |
| 421338 | AA287443 | | gb: zs52c10.r1 NCI_CGAP_GCB1 Homo sapiens | 7.45 | 0.09 | upregulate stage |
| 421508 | NM_004833 | Hs.105115 | absent in melanoma 2 | 4.21 | 0.19 | upregulate stage |
| 421634 | AA437414 | Hs.106283 | hypothetical protein FLJ10262 | 7.79 | 0.08 | upregulate stage |
| 421674 | T10707 | Hs.296355 | neuronal PAS domain protein 2 | 7.5 | 0.1 | upregulate stage |
| 421810 | AK001718 | Hs.108530 | hypothetical protein FLJ10856 | 8.45 | 0.08 | upregulate stage |
| 421855 | F06504 | Hs.27384 | ESTs | 2.9 | 0.16 | upregulate stage |
| 421898 | AA259011 | Hs.109268 | hypothetical protein FLJ12552 | 7.06 | 0.11 | upregulate stage |
| 422156 | N34524 | Hs.300893 | ESTs, Weakly similar to envelope protein | 3.75 | 0.16 | upregulate stage |
| 422225 | BE245652 | Hs.118281 | zinc finger protein 266 | 2.95 | 0.17 | upregulate stage |
| 422243 | AW803733 | Hs.250655 | prothymosin, alpha (gene sequence 28) | 8.15 | 0.08 | upregulate stage |
| 422511 | AU076442 | Hs.117938 | collagen, type XVII, alpha 1 | 2.21 | 0.17 | upregulate stage |
| 422634 | NM_016010 | Hs.118821 | CGI-62 protein | 1.3 | 0.29 | upregulate stage |
| 422988 | AW673847 | Hs.97321 | ESTs | 4.15 | 0.11 | upregulate stage |
| 423081 | AF262992 | Hs.123159 | sperm associated antigen 4 | 2.82 | 0.3 | upregulate stage |
| 423596 | AA328195 | Hs.234101 | ESTs, Weakly similar to CTL1 protein [H. | 2.75 | 0.19 | upregulate stage |
| 423872 | AB020316 | Hs.134015 | uronyl 2-sulfotransferase | 1.95 | 0.13 | upregulate stage |
| 423979 | AF229181 | Hs.136644 | CS box-containing WD protein | 7.12 | 0.11 | upregulate stage |
| 424005 | AB033041 | Hs.137507 | KIAA1215 protein | 1.71 | 0.37 | upregulate stage |
| 424014 | AA333653 | Hs.24790 | KIAA1573 protein | 4.85 | 0.12 | upregulate stage |
| 424028 | AF055084 | Hs.153692 | KIAA0686 protein | 8.5 | 0.07 | upregulate stage |
| 424194 | BE245833 | Hs.169854 | hypothetical protein SP 192 | 6.1 | 0.1 | upregulate stage |
| 424308 | AW975531 | Hs.154443 | minichromosome maintenance deficient (S. | 6.59 | 0.11 | upregulate stage |
| 424550 | AI650541 | Hs.115298 | ESTs | 3.25 | 0.12 | upregulate stage |
| 424631 | AA688021 | Hs.179808 | ESTs | 5.45 | 0.11 | upregulate stage |
| 424659 | AW891298 | Hs.301877 | ESTs, Weakly similar to hSIAH2 [H. sapien | 3.55 | 0.15 | upregulate stage |
| 424704 | AI263293 | Hs.152096 | cytochrome P450, subfamily IIJ (arachido | 8.45 | 0.06 | upregulate stage |
| 424775 | AB014540 | Hs.153026 | SWAP-70 protein | 6.65 | 0.11 | upregulate stage |
| 424800 | AL035588 | Hs.153203 | MyoD family inhibitor | 1.94 | 0.3 | upregulate stage |
| 425066 | M82882 | Hs.154365 | E74-like factor 1 (ets domain transcript | 2.85 | 0.19 | upregulate stage |
| 425259 | AL049280 | Hs.155397 | Homo sapiens mRNA; cDNA DKFZp564K143 (fr | 5.2 | 0.1 | upregulate stage |
| 425277 | NM_001241 | Hs.155478 | cyclin T2 | 6 | 0.13 | upregulate stage |
| 425508 | AA991551 | Hs.97013 | ESTs | 5.67 | 0.1 | upregulate stage |
| 425689 | W16480 | Hs.24283 | ESTs | 4.55 | 0.13 | upregulate stage |
| 425721 | AC002115 | Hs.159309 | uroplakin 1A | 0.71 | 0.8 | upregulate stage |
| 426069 | H10807 | Hs.30998 | ESTs | 3.4 | 0.17 | upregulate stage |
| 426088 | AF038007 | Hs.166196 | ATPase, Class I, type 8B, member 1 | 6.84 | 0.09 | upregulate stage |
| 426110 | NM_002913 | Hs.166563 | replication factor C (activator 1) 1 (14 | 6.7 | 0.1 | upregulate stage |
| 426227 | U67058 | Hs.168102 | Human proteinase activated receptor-2 mR | 3.05 | 0.14 | upregulate stage |
| 426603 | AA382291 | | gb: EST95683 Testis I Homo sapiens cDNA 5 | 1.8 | 0.26 | upregulate stage |
| 426657 | NM_015865 | Hs.171731 | solute carrier family 14 (urea transport | 0.96 | 0.36 | upregulate stage |
| 426716 | NM_006379 | Hs.171921 | sema domain, immunoglobulin domain (Ig), | 1.32 | 0.49 | upregulate stage |
| 426902 | AI125334 | Hs.97408 | ESTs | 5.05 | 0.07 | upregulate stage |
| 426931 | NM_003416 | Hs.2076 | zinc finger protein 7 (KOX 4, clone HF.1 | 2.95 | 0.12 | upregulate stage |
| 427001 | NM_006482 | Hs.173135 | dual-specificity tyrosine-(Y)-phosphoryl | 4.9 | 0.13 | upregulate stage |
| 427087 | BE073913 | Hs.173515 | uncharacterized hypothalamus protein HT0 | 0.57 | 0.86 | upregulate stage |
| 427398 | AW390020 | Hs.20415 | chromosome 21 open reading frame 11 | 1.86 | 0.31 | upregulate stage |
| 427399 | NM_014883 | Hs.177664 | KIAA0914 gene product | 5 | 0.13 | upregulate stage |
| 427450 | AB014526 | Hs.178121 | KIAA0626 gene product | 5.3 | 0.09 | upregulate stage |
| 427490 | Z95152 | Hs.178695 | mitogen-activated protein kinase 13 | 6.37 | 0.13 | upregulate stage |
| 427737 | AA435988 | Hs.178066 | ESTs, Weakly similar to AF068289 5 HDCME | 5.7 | 0.11 | upregulate stage |
| 428042 | AA419529 | | gb: zv03d12.r1 Soares_NhHMPu_S1 Homo sapi | 1.65 | 0.14 | upregulate stage |
| 428336 | AA503115 | Hs.183752 | microseminoprotein, beta- | 5.37 | 0.05 | upregulate stage |
| 428337 | AA644508 | | gb: af73c01.r1 Soares_NhHMPu_S1 Homo sapi | 3.4 | 0.13 | upregulate stage |
| 428365 | AA295331 | Hs.183861 | Homo sapiens cDNA FLJ20042 fis, clone CO | 1 | 0.17 | upregulate stage |
| 428471 | X57348 | Hs.184510 | stratifin | 1.81 | 0.39 | upregulate stage |
| 428583 | AA430589 | Hs.301374 | ESTs, Moderately similar to ALU5_HUMAN A | 7.55 | 0.11 | upregulate stage |
| 428670 | AA431682 | Hs.134832 | ESTs | 8.05 | 0.1 | upregulate stage |
| 428785 | AI015953 | Hs.125265 | ESTs | 1.65 | 0.15 | upregulate stage |
| 429332 | AF030403 | Hs.199263 | Ste-20 related kinase | 2.64 | 0.26 | upregulate stage |
| 429343 | AK000785 | Hs.199480 | epsin 3 | 3.15 | 0.27 | upregulate stage |
| 429556 | AW139399 | Hs.98988 | ESTs | 1.87 | 0.31 | upregulate stage |
| 429663 | M68874 | Hs.211587 | phospholipase A2, group IVA (cytosolic, | 0.61 | 1.02 | upregulate stage |
| 429824 | AA296363 | Hs.121520 | Human BAC clone GS1-99H8 | 2.03 | 0.39 | upregulate stage |
| 429966 | BE081342 | Hs.226799 | HSPC039 protein | 7.85 | 0.08 | upregulate stage |
| 429970 | AK000072 | Hs.227059 | chloride channel, calcium activated, fam | 1 | 0.61 | upregulate stage |
| 430042 | AB023170 | Hs.227850 | KIAA0953 protein | 2.5 | 0.17 | upregulate stage |
| 430168 | AW968343 | Hs.300896 | ESTs, Highly similar to AF128113 1 promi | 1.98 | 0.4 | upregulate stage |
| 430308 | BE540865 | Hs.238990 | cyclin-dependent kinase inhibitor 18 (p2 | 6.7 | 0.09 | upregulate stage |
| 430399 | AI916284 | Hs.199671 | ESTs | 8.09 | 0.08 | upregulate stage |
| 430763 | AA485468 | Hs.105658 | ESTs | 3.18 | 0.24 | upregulate stage |
| 431474 | AL133990 | Hs.190642 | ESTs | 0.37 | 0.51 | upregulate stage |
| 431567 | N51357 | Hs.260855 | Homo sapiens mRNA; cDNA DKFZp761G2311 (f | 1.74 | 0.39 | upregulate stage |
| 431585 | BE242803 | Hs.262823 | hypothetical protein FLJ10326 | 3.55 | 0.15 | upregulate stage |
| 431683 | AK001749 | Hs.267604 | hypothetical protein FLJ10450 | 8.55 | 0.08 | upregulate stage |
| 431709 | AF220185 | Hs.267923 | uncharacterized hypothalamus protein HT0 | 7.95 | 0.1 | upregulate stage |
| 431846 | BE019924 | Hs.271580 | uroplakin 1B | 1.33 | 0.5 | upregulate stage |
| 431912 | AI660552 | Hs.154903 | ESTs, Weakly similar to A56154 AbI subst | 4.15 | 0.12 | upregulate stage |
| 432350 | NM_005865 | Hs.274407 | protease, serine, 16 (thymus) | 3.7 | 0.13 | upregulate stage |
| 432520 | AI075978 | Hs.188007 | ESTs | 2.05 | 0.22 | upregulate stage |
| 432524 | AI458020 | Hs.293287 | ESTs | 5.15 | 0.14 | upregulate stage |
| 432540 | AI821517 | Hs.105866 | ESTs | 5.5 | 0.11 | upregulate stage |
| 432623 | AA557351 | Hs.152448 | ESTs, Moderately similar to PUR6_HUMAN M | 8.43 | 0.09 | upregulate stage |
| 432632 | AW973801 | Hs.134656 | ESTs | 2.45 | 0.16 | upregulate stage |
| 432820 | AI554057 | Hs.152477 | ESTs | 8.29 | 0.09 | upregulate stage |
| 432945 | AL043683 | Hs.271357 | ESTs, Weakly similar to unnamed protein | 3.22 | 0.23 | upregulate stage |
| 433027 | AF191018 | Hs.279923 | putative nucleotide binding protein, est | 2.15 | 0.39 | upregulate stage |
| 433037 | NM_014158 | Hs.279938 | HSPC067 protein | 5.1 | 0.11 | upregulate stage |
| 433156 | R59206 | Hs.17519 | Homo sapiens cDNA: FLJ22539 fis, clone H | 7.9 | 0.1 | upregulate stage |
| 433171 | AA579425 | | gb: nf37c08.s1 NCI_CGAP_Pr2 Homo sapiens | 3.54 | 0.14 | upregulate stage |
| 433311 | AA688149 | | gb: nv16h12.s1 NCI_CGAP_Pr22 Homo sapiens | 6.6 | 0.08 | upregulate stage |
| 433383 | AF034837 | Hs.192731 | double-stranded RNA specific adenosine d | 2.45 | 0.21 | upregulate stage |
| 433409 | AI278802 | Hs.25661 | ESTs | 4.75 | 0.1 | upregulate stage |
| 433650 | AA603472 | Hs.28456 | ESTs | 1.6 | 0.18 | upregulate stage |
| 433675 | AW977653 | Hs.110771 | Homo sapiens cDNA: FLJ21904 fis, clone H | 3.88 | 0.17 | upregulate stage |
| 434328 | BE564937 | Hs.15984 | pp21 homolog | 3 | 0.15 | upregulate stage |
| 434476 | AW858520 | Hs.271825 | ESTs | 4.6 | 0.1 | upregulate stage |
| 434683 | AW298724 | Hs.202639 | ESTs | 2.1 | 0.19 | upregulate stage |
| 434726 | AF062719 | Hs.139053 | ESTs | 1.76 | 0.34 | upregulate stage |
| 435124 | AA725362 | Hs.120456 | ESTs | 7.7 | 0.09 | upregulate stage |
| 435563 | AF210317 | Hs.95497 | solute carrier family 2 (facilitated glu | 5.8 | 0.09 | upregulate stage |
| 435899 | W89093 | Hs.189914 | ESTs | 1.32 | 0.42 | upregulate stage |
| 436026 | AI349764 | Hs.217081 | ESTs | 1 | 0.22 | upregulate stage |
| 436154 | AA764950 | Hs.119898 | ESTs | 8.4 | 0.05 | upregulate stage |
| 436293 | AI601188 | Hs.120910 | ESTs | 2.42 | 0.2 | upregulate stage |
| 436361 | AA825814 | Hs.149065 | ESTs | 6.95 | 0.09 | upregulate stage |
| 436455 | AI027959 | Hs.132300 | ESTs | 3.25 | 0.15 | upregulate stage |
| 436577 | W84774 | Hs.17643 | ESTs | 6.3 | 0.06 | upregulate stage |
| 436684 | AW976319 | Hs.94806 | KIAA1062 protein | 4.75 | 0.12 | upregulate stage |
| 437036 | AI571514 | Hs.133022 | ESTs | 1.4 | 0.13 | upregulate stage |
| 437146 | AA730977 | | gb: nw55f05.s1 NCI_CGAP_Ew1 Homo sapiens | 1 | 0.37 | upregulate stage |
| 437262 | BE250537 | Hs.174838 | Homo sapiens cDNA FLJ14192 fis, clone NT | 3.25 | 0.17 | upregulate stage |
| 437277 | AA748016 | Hs.123370 | ESTs | 6.75 | 0.09 | upregulate stage |
| 437882 | AI243203 | Hs.131572 | ESTs | 8.12 | 0.09 | upregulate stage |
| 438392 | AA806395 | Hs.123205 | ESTs | 1 | 0.34 | upregulate stage |
| 438416 | N76398 | Hs.21187 | Homo sapiens cDNA: FLJ23068 fis, clone L | 8.1 | 0.1 | upregulate stage |
| 438739 | AA815391 | | gb: ai61c02.s1 Soares_testis_NHT Homo sap | 4.69 | 0.12 | upregulate stage |
| 439211 | AI890347 | Hs.271923 | Homo sapiens cDNA: FLJ22785 fis, clone K | 6.65 | 0.11 | upregulate stage |
| 439394 | AA149250 | Hs.56105 | ESTs, Weakly similar to WDNM RAT WDNM1 P | 3.19 | 0.11 | upregulate stage |
| 439544 | W26354 | Hs.28891 | hypothetical protein FLJ11360 | 2.3 | 0.34 | upregulate stage |
| 439569 | AW602166 | Hs.222399 | CEGP1 protein | 0.73 | 0.51 | upregulate stage |
| 439586 | AA922936 | Hs.110039 | ESTs | 4.3 | 0.1 | upregulate stage |
| 439706 | AW872527 | Hs.59761 | ESTs | 1 | 0.14 | upregulate stage |
| 439897 | NM_015310 | Hs.6763 | KIAA0942 protein | 8.4 | 0.08 | upregulate stage |
| 439898 | AW505514 | Hs.209561 | ESTs, Weakly similar to C05E11.1 gene pr | 7.35 | 0.1 | upregulate stage |
| 439949 | AW979197 | Hs.292073 | ESTs | 8.55 | 0.08 | upregulate stage |
| 440035 | BE561589 | Hs.285122 | hypothetical protein FLJ21839 | 6 | 0.11 | upregulate stage |
| 440619 | AW408586 | Hs.91052 | ESTs, Moderately similar to ALU5_HUMAN A | 7.95 | 0.07 | upregulate stage |
| 440635 | AW610331 | | gb: RC4-ST0316-190100-011-c08 ST0316 Homo | 5.95 | 0.11 | upregulate stage |
| 440787 | AW292043 | Hs.209433 | ESTs | 5.05 | 0.12 | upregulate stage |
| 441233 | AA972965 | Hs.135568 | ESTs | 1.7 | 0.12 | upregulate stage |
| 441528 | AI003797 | Hs.130815 | hypothetical protein FLJ21870 | 7.2 | 0.09 | upregulate stage |
| 441670 | AW874090 | Hs.127392 | ESTs, Moderately similar to p33ING1 [H. s | 2.45 | 0.19 | upregulate stage |
| 441683 | BE564214 | Hs.102946 | ESTs | 5.9 | 0.13 | upregulate stage |
| 441847 | AI215564 | Hs.220972 | ESTs | 6.95 | 0.11 | upregulate stage |
| 442145 | AI022650 | Hs.8117 | erbb2-interacting protein ERBIN | 3 | 0.19 | upregulate stage |
| 442299 | AW467791 | Hs.155561 | ESTs | 5.05 | 0.13 | upregulate stage |
| 442315 | AA173992 | Hs.7956 | ESTs | 3.97 | 0.17 | upregulate stage |
| 442528 | AF150317 | Hs.134217 | ESTs | 1.4 | 0.34 | upregulate stage |
| 442571 | C06338 | Hs.165464 | ESTs | 8 | 0.08 | upregulate stage |
| 442607 | AA507576 | Hs.288361 | Homo sapiens cDNA: FLJ22696 fis, clone H | 6.7 | 0.1 | upregulate stage |
| 442652 | AI005163 | Hs.201378 | ESTs, Weakly similar to KIAA0944 protein | 7.15 | 0.11 | upregulate stage |
| 442947 | R40800 | Hs.21303 | ESTs | 8.5 | 0.08 | upregulate stage |
| 442993 | BE018682 | Hs.44343 | ESTs | 1.91 | 0.34 | upregulate stage |
| 443015 | R33261 | Hs.6614 | ESTs | 8.5 | 0.09 | upregulate stage |
| 443085 | AI032660 | Hs.164711 | ESTs | 4 | 0.13 | upregulate stage |
| 443228 | W24781 | Hs.293798 | ESTs | 1.61 | 0.47 | upregulate stage |
| 443367 | AW071349 | Hs.215937 | ESTs | 1.75 | 0.29 | upregulate stage |
| 443371 | AI792888 | Hs.145489 | ESTs | 5.85 | 0.11 | upregulate stage |
| 443564 | AI921685 | Hs.199713 | ESTs | 1.4 | 0.18 | upregulate stage |
| 443638 | AW028696 | Hs.145679 | ESTs | 3.25 | 0.15 | upregulate stage |
| 443677 | AV646096 | Hs.293776 | ESTs, Weakly similar to 1207289A reverse | 6.45 | 0.11 | upregulate stage |
| 443861 | AW449462 | Hs.134743 | ESTs | 6.72 | 0.09 | upregulate stage |
| 444097 | AW517412 | Hs.150757 | ESTs | 4.25 | 0.11 | upregulate stage |
| 444171 | AB018249 | Hs.10458 | small inducible cytokine subfamily A (Cy | 8.45 | 0.09 | upregulate stage |
| 444184 | T87841 | Hs.282990 | Human DNA sequence from clone RP1-28H20 | 8.1 | 0.1 | upregulate stage |
| 444385 | BE278964 | Hs.11085 | CGI-111 protein | 8.6 | 0.09 | upregulate stage |
| 444624 | AV650476 | Hs.282936 | ESTs | 7.52 | 0.1 | upregulate stage |
| 444631 | AW995395 | Hs.84520 | ESTs | 1.25 | 0.21 | upregulate stage |
| 444707 | AI188613 | Hs.143866 | ESTs | 2.1 | 0.21 | upregulate stage |
| 444735 | BE019923 | Hs.243122 | hypothetical protein FLJ13057 similar to | 6.8 | 0.1 | upregulate stage |
| 444779 | AI192105 | Hs.147170 | ESTs | 0.94 | 0.6 | upregulate stage |
| 444823 | BE262989 | Hs.12045 | putative protein | 8.09 | 0.1 | upregulate stage |
| 444858 | AI199738 | Hs.208275 | ESTs, Weakly similar to unnamed protein | 4.6 | 0.09 | upregulate stage |
| 444875 | AI200759 | Hs.44737 | ESTs | 6.85 | 0.11 | upregulate stage |
| 444888 | AI651039 | Hs.148559 | ESTs | 3.15 | 0.18 | upregulate stage |
| 445076 | AI206888 | Hs.154131 | ESTs | 7.81 | 0.09 | upregulate stage |
| 445182 | AW189787 | Hs.147474 | ESTs | 2 | 0.07 | upregulate stage |
| 445189 | AI936450 | Hs.147482 | ESTs | 2.65 | 0.12 | upregulate stage |
| 445320 | AA503887 | Hs.167011 | Homo sapiens cDNA: FLJ21362 fis, clone C | 1.47 | 0.46 | upregulate stage |
| 445594 | AW058463 | Hs.12940 | zinc-fingers and homeoboxes 1 | 6.2 | 0.07 | upregulate stage |
| 445674 | BE410347 | Hs.13063 | transcription factor CA150 | 3.8 | 0.15 | upregulate stage |
| 445817 | NM_003642 | Hs.13340 | histone acetyltransferase 1 | 5.6 | 0.1 | upregulate stage |
| 445871 | AI702901 | Hs.145582 | ESTs | 2.3 | 0.33 | upregulate stage |
| 446140 | AA356170 | Hs.26750 | Homo sapiens cDNA: FLJ21908 fis, clone H | 2.15 | 0.18 | upregulate stage |
| 446553 | AB021179 | Hs.15299 | HMBA-inducible | 2.55 | 0.18 | upregulate stage |
| 446651 | AA393907 | Hs.97179 | ESTs | 8.05 | 0.07 | upregulate stage |
| 447086 | AI421397 | Hs.161321 | ESTs | 6.9 | 0.1 | upregulate stage |
| 447290 | AI476732 | Hs.263912 | ESTs | 2.35 | 0.18 | upregulate stage |
| 447379 | AI554946 | Hs.158794 | ESTs | 6.3 | 0.09 | upregulate stage |
| 447390 | X95384 | Hs.18426 | translational inhibitor protein p14.5 | 7.25 | 0.08 | upregulate stage |
| 447533 | NM_004786 | Hs.18792 | thioredoxin-like, 32 kD | 1 | 0.24 | upregulate stage |
| 447548 | N53388 | Hs.7222 | ESTs | 8.6 | 0.07 | upregulate stage |
| 447731 | AA373527 | Hs.19385 | CGI-58 protein | 7.3 | 0.08 | upregulate stage |
| 447853 | AI434204 | Hs.164285 | ESTs, Weakly similar to Afg1p [S. cerevis | 6.75 | 0.11 | upregulate stage |
| 447857 | AA081218 | Hs.58608 | Homo sapiens cDNA FLJ14206 fis, clone NT | 2.2 | 0.24 | upregulate stage |
| 447965 | AW292577 | Hs.94445 | ESTs | 3.6 | 0.13 | upregulate stage |
| 448072 | AI459306 | Hs.24908 | ESTs | 5.8 | 0.11 | upregulate stage |
| 448474 | AI792014 | Hs.13809 | ESTs | 2.72 | 0.28 | upregulate stage |
| 448513 | AA344741 | Hs.61773 | Homo sapiens cDNA FLJ11648 fis, clone HE | 4.8 | 0.12 | upregulate stage |
| 448601 | R61666 | Hs.293690 | ESTs | 2.65 | 0.2 | upregulate stage |
| 448625 | AW970786 | Hs.178470 | Homo sapiens cDNA: FLJ22662 fis, clone H | 1.68 | 0.44 | upregulate stage |
| 448735 | AW473830 | Hs.171442 | ESTs | 2.95 | 0.19 | upregulate stage |
| 448807 | AI571940 | Hs.7549 | ESTs | 2.3 | 0.14 | upregulate stage |
| 448920 | AW408009 | Hs.22580 | alkylglycerone phosphate synthase | 8.6 | 0.08 | upregulate stage |
| 449448 | D60730 | Hs.57471 | ESTs | 1 | 0.13 | upregulate stage |
| 449517 | AW500106 | Hs.23643 | serine/threonine protein kinase MASK | 6.4 | 0.11 | upregulate stage |
| 449585 | AI655321 | Hs.197693 | ESTs | 1 | 0.16 | upregulate stage |
| 449619 | AI655992 | Hs.300647 | ESTs | 8.35 | 0.09 | upregulate stage |
| 449659 | R60031 | Hs.198899 | eukaryotic translation initiation factor | 6.65 | 0.11 | upregulate stage |
| 449689 | AF228421 | Hs.301039 | Human DNA sequence from clone RP1-132F21 | 8.35 | 0.06 | upregulate stage |
| 449901 | AI674072 | | gb: wd15h01.x1 Soares_NFL_T_GBC_S1 Homo s | 5.8 | 0.1 | upregulate stage |
| 449964 | AW001741 | Hs.273193 | hypothetical protein FLJ10706 | 8.7 | 0.09 | upregulate stage |
| 450170 | AI685366 | Hs.32775 | ESTs | 6.77 | 0.12 | upregulate stage |
| 450193 | AI916071 | Hs.224623 | ESTs | 5.73 | 0.1 | upregulate stage |
| 450336 | AA046814 | Hs.288928 | Homo sapiens cDNA: FLJ23296 fis, clone H | 8.2 | 0.08 | upregulate stage |
| 450341 | N90956 | Hs.17230 | hypothetical protein FLJ22087 | 4.2 | 0.19 | upregulate stage |
| 450353 | AI244661 | Hs.103296 | ESTs | 4.71 | 0.15 | upregulate stage |
| 450737 | AW007152 | Hs.203330 | ESTs | 2.14 | 0.25 | upregulate stage |
| 450795 | AW173371 | Hs.60435 | ESTs | 6 | 0.1 | upregulate stage |
| 450928 | AI744417 | | gb: tr10h12.x1 NCI_CGAP_Ov23 Homo sapiens | 1.75 | 0.18 | upregulate stage |
| 451134 | AA318315 | Hs.25999 | hypothetical protein FLJ22195 | 4.3 | 0.1 | upregulate stage |
| 451230 | BE546208 | Hs.26090 | hypothetical protein FLJ20272 | 4.75 | 0.16 | upregulate stage |
| 451593 | AF151879 | Hs.26706 | CGI-121 protein | 5.8 | 0.11 | upregulate stage |
| 451618 | AA115639 | Hs.26764 | KIAA0546 protein | 5.8 | 0.13 | upregulate stage |
| 451668 | Z43948 | Hs.26789 | hypothetical protein FLJ10320 | 0.73 | 0.26 | upregulate stage |
| 451790 | AA927403 | Hs.43897 | ESTs, Weakly similar to P2CA_HUMAN PROTE | 3.2 | 0.25 | upregulate stage |
| 452001 | AI827675 | Hs.297735 | Homo sapiens cDNA: FLJ22094 fis, clone H | 3.7 | 0.13 | upregulate stage |
| 452039 | AI922988 | Hs.172510 | ESTs | 1 | 0.65 | upregulate stage |
| 452046 | AB018345 | Hs.27657 | KIAA0802 protein | 1.13 | 0.39 | upregulate stage |
| 452092 | BE245374 | Hs.27842 | hypothetical protein FLJ11210 | 3.2 | 0.15 | upregulate stage |
| 452278 | AL037715 | Hs.28785 | Homo sapiens mRNA; cDNA DKFZp586F0219 (f | 8.25 | 0.07 | upregulate stage |
| 452381 | H23329 | Hs.290880 | ESTs, Weakly similar to ALU1_HUMAN ALU S | 1 | 0.34 | upregulate stage |
| 452420 | BE564871 | Hs.29463 | centrin, EF-hand protein, 3 (CDC31 yeast | 4.97 | 0.13 | upregulate stage |
| 452714 | AW770994 | Hs.30340 | hypothetical protein KIAA1165 | 7.6 | 0.09 | upregulate stage |
| 453078 | AF053551 | Hs.31584 | metaxin 2 | 5.3 | 0.09 | upregulate stage |
| 453370 | AI470523 | Hs.182356 | ESTs, Moderately similar to translation | 3.78 | 0.13 | upregulate stage |
| 453765 | BE279901 | Hs.35091 | hypothetical protein FLJ10775 | 3.95 | 0.11 | upregulate stage |
| 453972 | AW137224 | Hs.245869 | ESTs | 6 | 0.09 | upregulate stage |
| 454044 | AW022393 | | gb: df37h12.y1 Morton Fetal Cochlea Homo | 1.15 | 0.18 | upregulate stage |
| 454289 | AL137554 | Hs.49927 | Homo sapiens mRNA; cDNA DKFZp434H1720 (f | 7.05 | 0.1 | upregulate stage |
| 454314 | AW364844 | | gb: QV3-DT0044-221299-045-c03 DT0044 Homo | 1 | 0.37 | upregulate stage |
| 454315 | AW373564 | Hs.251928 | nuclear pore complex interacting protein | 2.7 | 0.13 | upregulate stage |
| 454775 | BE160229 | | gb: QV1-HT0413-090200-062-a12 HT0413 Homo | 8.5 | 0.09 | upregulate stage |
| 454790 | AW820852 | | gb: RC2-ST0301-120200-011-f12 ST0301 Homo | 1.15 | 0.14 | upregulate stage |
| 454792 | AW820794 | Hs.252406 | hypothetical protein FLJ12296 similar to | 3.65 | 0.12 | upregulate stage |
| 455170 | AW860972 | | gb: QV0-CT0387-180300-167-h07 CT0387 Homo | 5.7 | 0.07 | upregulate stage |
| 455511 | BE144762 | | gb: CM0-HT0180-041099-065-b04 HT0180 Homo | 1 | 0.25 | upregulate stage |
| 456141 | AI751357 | Hs.288741 | Homo sapiens cDNA: FLJ22256 fis, clone H | 8.35 | 0.09 | upregulate stage |
| 456258 | AW976410 | Hs.289069 | Homo sapiens cDNA: FLJ21016 fis, clone C | 4.85 | 0.14 | upregulate stage |
| 456279 | AW006783 | Hs.6686 | ESTs | 7.25 | 0.1 | upregulate stage |
| 457518 | AA825350 | Hs.143805 | ESTs, Weakly similar to ALU1_HUMAN ALU S | 6.84 | 0.11 | upregulate stage |
| 457570 | AA579426 | Hs.190226 | ESTs | 2.6 | 0.2 | upregulate stage |
| 457982 | AW856093 | Hs.183617 | ESTs | 1 | 0.25 | upregulate stage |
| 458080 | BE142728 | | gb: MR0-HT0157-021299-004-d08 HT0157 Homo | 2.05 | 0.27 | upregulate stage |
| 458340 | AI457102 | Hs.121583 | Human glucose transporter pseudogene | 2.25 | 0.18 | upregulate stage |
| 458440 | AI095468 | Hs.135254 | ESTs, Weakly similar to thrombospondin t | 2.35 | 0.13 | upregulate stage |
| 458771 | AW295151 | Hs.163612 | ESTs | 1 | 0.19 | upregulate stage |
| 459092 | AA722012 | Hs.255757 | ESTs, Weakly similar to KIAA0611 protein | 6.95 | 0.1 | upregulate stage |

Pkey: Unique Eos probeset identifier number
ExAccn: Exemplar Accession number, Genbank accession number
UnigeneID: Unigene number
Unigene Title: Unigene gene title
R1: please refer to original application
R2: please refer to original application
Target Type: downregulate stage if target is downregulated in bladder tumors relative to normal bladder or early stage if target is an earl stage (Ta) bladder tumor marker or late stage if target is a late stage (T2-T4) bladder tumor marker or T2-T4 grade 3 papilloma marker or T2-T4 grade 3 solid tumor marker or Upregulate stage

TABLE 1B

| Pkey | CAT | Accessions |
|---|---|---|
| 407774 | 101538_1 | AA084958 AA214034 AA044587 AA130152 AA130116 D81924 |
| 407939 | 1027688_1 | W05608 AW118352 AW196215 |
| 407980 | 103087_1 | AA046309 AI263500 AA046397 |
| 408224 | 1048369_1 | AW175997 AW176000 AW175999 AW175994 AW176004 AW175989 |
| 408241 | 1048867_1 | AW176546 AW178965 BE141057 |
| 408268 | 104987_1 | AL138247 AA053529 BE173313 |
| 408277 | 1050131_1 | AW177959 AW177960 AW178066 AW178022 |
| 408306 | 1050863_1 | BE141991 AW178855 BE141990 BE141993 BE141994 BE062405 BE062404 AW178850 |
| 409281 | 111502_1 | AA069998 AA075659 AA075660 AA069828 |
| 409408 | 1128045_1 | AW387837 AW387874 AW387826 AW387758 AW387864 AW809268 AW387754 |
| 409566 | 114012_1 | AA078899 AA078782 AA075788 |
| 409760 | 115373_1 | AA302840 T93016 T92950 AA077551 |
| 409794 | 1154785_1 | AW885691 BE185989 BE185940 AW500322 H10282 |
| 409810 | 1155339_1 | AW500895 AW937549 |
| 409840 | 1156071_1 | AW502122 AW502125 AW501663 AW501720 |
| 410071 | 1174764_1 | AW582568 AW818656 AW818647 AW818655 AW818637 AW818234 |
| 410128 | 117773_1 | AW904599 AA325920 AW964919 AA081786 T05150 |
| 410269 | 1189983_1 | AW613597 BE080235 BE080168 |
| 410475 | 1204788_1 | AW749927 AW749938 AW749986 AW749966 R87124 AW860547 |
| 410495 | 1205826_1 | N95428 W24040 AW751366 H81987 |
| 410520 | 1206965_1 | AW752710 BE180336 BE180186 |
| 410534 | 1207247_1 | AW905138 AW753008 R13818 Z43519 |
| 410537 | 1207336_1 | AW753108 AW852909 N36993 AW894327 AW903629 |
| 410560 | 1208311_1 | N29220 AW754225 AW754224 |
| 410562 | 1208415_1 | AW858528 AW754362 H74000 |
| 410724 | 1218202_1 | AW799269 AW799385 AW799501 AW799386 AW799270 AW799467 |
| 410751 | 121964_1 | AA357918 AA089516 AA357917 |
| 410754 | 1219733_1 | T63840 AW801569 AW801568 |
| 410785 | 1221055_1 | AW803341 AW803265 AW803403 AW803466 AW803402 AW803413 AW803268 AW803396 AW803334 AW803355 |
| 410844 | 1223878_1 | AW807073 AW807055 AW807067 AW807276 AW807030 AW807363 AW845892 AW807091 AW807275 AW807284 |
| | | AW807287 AW845891 AW807195 AW807271 |
| 410910 | 1226517_1 | AW810204 AW810555 AW810196 AW810619 AW810507 |
| 410973 | 1228236_1 | AW812278 AW812286 AW812274 |
| 410997 | 1228736_1 | AW812877 AW812878 AW812952 AW812880 |
| 411110 | 1232598_1 | H93000 AW818022 AW818044 |
| 411112 | 1232692_1 | AW818158 AW865743 AW865702 AW865698 |
| 411132 | 1233580_1 | AW819191 AW819252 AW819183 AW819175 AW819177 AW819186 AW819180 BE158470 AW819242 |
| | | AW819269 AW819244 AW819190 AW819265 AW819268 AW819246 BE152602 AW819249 AW819251 AW819263 |
| | | AW819194 |
| 411137 | 1233775_1 | AW819455 AW819711 AW819553 |
| 411157 | 1234099_1 | AW819867 AW819956 AW820159 |
| 411159 | 1234102_1 | AW820178 AW820183 AW819870 AW819937 AW835709 AW820184 AW820157 AW819945 AW820096 |
| 411162 | 1234161_1 | AW819944 AW820182 AW820168 AW819959 AW819953 |
| 411170 | 1234379_1 | AW820503 AW820306 AW820429 BE174741 AW820244 BE174738 |
| 411173 | 123446_1 | R81571 AW962014 AA324277 |
| 411193 | 1235254_1 | AW821484 AW821461 AW821490 AW821525 AW821526 AW821519 |
| 411242 | 1236393_1 | BE146808 BE146949 AW833828 BE147002 BE147066 AW833422 AW833475 AW833485 AW833414 |
| | | AW833406 AW833395 AW833640 AW833835 AW833421 BE146805 AW833465 BE146753 BE147004 AW833473 |
| | | AW833573 BE147068 AW833466 AW833680 BE147063 BE147058 BE146807 AW833830 AW833694 AW833596 |
| | | AW833601 |
| 411245 | 1236412_1 | AW833441 AW833552 AW833700 AW833610 AW833673 AW833675 |
| 411282 | 1237660_1 | AW995011 AW880630 AW995662 AW880196 AW860455 AW995379 AW880634 AW938253 AW880580 |
| | | AW938263 AW880584 AW860454 AW880603 AW880026 AW938265 AW880592 AW852203 AW880588 AW880680 |
| | | AW995392 AW880598 AW860603 AW880590 AW880175 AW846534 AW880098 AW880108 AW860453 AW938051 |
| | | AW995191 AW880532 AW880542 AW938197 AW994929 AW880635 AW835438 AW938057 AW938054 |
| 411327 | 1238874_1 | AW836922 AW876719 AW876688 AW836919 AW836997 AW836908 AW836912 AW836993 |
| 411336 | 1239216_1 | AW837675 AW837356 AW837581 AW837348 AW837681 |
| 411339 | 1239387_1 | BE164598 AW837578 AW837582 |
| 411407 | 1244071_1 | R00903 R20424 AW844169 |
| 411418 | 124518_1 | BE241870 AI752584 Z19223 AA307961 AA181165 AA093272 AW368570 |
| 411425 | 1245503_1 | AW846012 AW846007 AW845996 AW845975 |
| 411461 | 1246730_1 | AW847937 AW847672 AW847809 AW847923 AW847808 AW847616 |
| 411474 | 1247047_2 | AW848427 AW848890 AW848159 AW848118 AW848634 AW848285 AW848086 AW848485 AW848283 AW848162 |
| 411526 | 1248748_1 | AW850327 AW850350 AW850348 AW850375 |
| 411560 | 1249443_1 | AW851186 AW996967 BE143456 |
| 411568 | 1249776_1 | BE144593 AW851632 AW968149 AW968144 W21059 |
| 411691 | 1254166_1 | AW857199 BE066018 BE065982 BE066082 BE065949 BE066019 BE066081 BE065954 BE066084 |
| 411727 | 1255052_1 | AW858443 AW858436 AW861923 AW858551 AW861872 AW858474 AW861904 AW858398 |
| 411741 | 1255976_1 | AW859650 BE071806 AW859655 |
| 411771 | 1257335_1 | AW993247 AW861464 |
| 411787 | 1258789_1 | AW863568 BE161696 BE161629 BE161824 |
| 411788 | 125879_1 | AW897793 R20054 H23321 |
| 411826 | 1260006_1 | AW947946 AW947986 AW947977 AW865187 AW865246 AW865182 AW865376 AW865392 AW865184 |
| 411840 | 1260678_1 | AW866330 AW866410 AW866332 AW866411 AW866327 AW866326 AW866335 AW866334 AW866331 |
| | | AW866333 AW866328 AW866329 |
| 411860 | 126168_1 | T89420 N87510 AA095932 |
| 411880 | 1263110_1 | AW872477 BE088101 T05990 |
| 411932 | 1266125_1 | AW876548 AW876577 AW876528 AW876623 AW876519 AW876540 AW876569 AW876534 AW876563 AW876545 |
| | | AW876574 |
| 412134 | 1279147_1 | AW895560 AW895557 AW895314 AW895537 AW895287 AW895411 |
| 412178 | 1281471_1 | AW898526 AW898525 Z19700 |
| 412296 | 1288043_1 | AW936233 AW936272 |
| 412303 | 1288130_1 | AW936336 AW936339 |
| 412305 | 1288141_1 | AW936369 AW936377 AW936355 AW936410 AW936460 |
| 412312 | 1288349_1 | AW936686 AW936656 AW936608 AW936635 AW936652 AW936679 AW936726 AW936602 AW936733 |
| 412327 | 1288935_1 | AW937355 AW937450 BE162340 BE162397 |
| 412354 | 1290342_1 | AW939148 AW939200 BE161819 |
| 412357 | 1290611_1 | AW939537 AW939584 W92705 |
| 412359 | 129085_1 | AW837985 AW837938 AA101955 AW837913 AW837935 |
| 412367 | 1291505_1 | AW945964 AW946020 AW946034 AW946027 AW946041 AW946044 AW946033 AW946024 AW946021 AW946029 |
| | | AW946015 AW946016 AW946039 AW946045 AW946028 AW946036 |
| 412389 | 1292588_1 | AW947655 AW984020 |
| 412547 | 1305813_1 | W27161 AW961828 |
| 412559 | 1306011_1 | T31474 T31123 Z45146 AW962040 |
| 412671 | 1319959_1 | AW977734 H62048 |
| 412744 | 1324622_1 | N31101 N46491 AW994084 |
| 412838 | 1331178_1 | D61870 D61866 BE002674 |
| 413119 | 1349658_1 | BE065941 BE066098 BE066070 BE065997 BE066003 |
| 413141 | 1350477_1 | BE166323 BE067045 |
| 413266 | 1356260_1 | BE300352 BE299274 BE075351 BE297444 |
| 413294 | 1359149_1 | BE144034 N72932 BE080176 |
| 413342 | 136279_1 | AA128535 AW973587 AA553822 |
| 413402 | 1366932_1 | T24065 BE092527 BE092528 BE092204 BE092271 BE092516 BE092202 BE092280 BE092065 BE091991 BE092284 |
| | | BE092514 BE091995 BE092063 BE092068 BE091994 BE092283 BE092447 |
| 413445 | 1370833_1 | BE141022 BE141513 BE141532 BE141488 BE141481 BE141526 BE141516 BE141471 BE141478 BE141476 |
| | | BE141475 BE141021 BE141533 BE141464 BE141490 BE141472 BE141480 |
| 413485 | 1372953_1 | N52628 BE143639 BE143706 |
| 413508 | 1374273_1 | BE145364 BE145429 BE145418 |
| 413618 | 1380384_1 | BE154078 BE154006 BE153891 BE153523 BE153983 |
| 413707 | 1384137_1 | BE158679 BE158714 |
| 413743 | 1385884_1 | BE161004 BE162497 BE161135 BE160999 BE162492 |
| 413782 | 1389068_1 | BE546104 BE165710 BE382801 |
| 413792 | 1389912_1 | BE166924 BE166921 BE166925 BE166915 BE166970 BE166968 |
| 413804 | 1390710_1 | T64682 BE168190 BE168256 |
| 414091 | 141621_1 | T83742 AW672882 AA135378 |
| 414203 | 1425510_2 | BE262170 BE382553 BE261026 BE273627 |
| 414220 | 1426940_1 | BE298094 BE267860 |
| 414276 | 1432115_−1 | BE297862 |
| 414366 | 1438636_1 | BE549143 BE390613 BE277344 |
| 414833 | 1496271_1 | T07114 BE543688 |
| 414985 | 1511215_1 | C17372 D63180 D78908 D63262 |
| 415027 | 1515978_1 | D31010 D30991 D31168 D31166 D31465 |
| 415104 | 1522649_1 | D60076 D60259 D61037 |
| 415114 | 1523035_1 | D60468 D60950 D61218 N98936 |
| 415178 | 1527354_1 | D80503 D81739 N95691 |
| 415296 | 1533528_1 | F05086 F05091 R17158 |
| 415303 | 153360_1 | R11813 AA164376 T56632 |
| 415352 | 1534744_1 | F06565 Z43466 R18417 F06477 F06476 F07098 |
| 415364 | 1535008_1 | F06771 H04895 Z42778 |
| 415371 | 1535066_1 | R15239 Z45189 F06836 |
| 415392 | 1535746_1 | Z44067 F07617 R34555 |
| 415569 | 1539911_1 | Z43930 F12170 H10881 T65261 |
| 415572 | 1539951_1 | F12294 F12213 T66272 T66261 |
| 415600 | 1540373_1 | F12664 T74312 N80318 Z20039 |
| 415626 | 1540758_1 | Z43847 F13068 T75331 |
| 415635 | 1540853_1 | F13168 R21289 T77628 |
| 415750 | 155215_1 | AA167712 AW936024 AW364438 AW364446 |
| 415773 | 1554447_1 | R21651 H00542 |
| 415822 | 155791_1 | D59243 D63202 AA169716 |
| 415948 | 156264_1 | AA262226 AI984175 AA171807 |
| 416173 | 1574973_1 | R52782 R17313 H24192 R19876 |
| 416343 | 1589633_1 | H49213 W90725 H49398 W90726 |
| 416395 | 1592777_1 | R94575 T99886 H52989 |
| 416624 | 1604694_1 | H69044 T47567 H75691 T50292 |
| 417304 | 166556_1 | H15635 H16691 AA195506 |
| 417396 | 1674485_1 | T98987 T96744 N78306 |
| 417742 | 1696282_1 | R64719 Z44680 R12451 |
| 417974 | 171237_1 | AA210765 T95700 H94407 |
| 418297 | 1736343_1 | R91254 T97156 R58711 |
| 418454 | 175699_1 | AA315308 AA223392 BE538098 BE087173 |
| 418498 | 1762961_1 | T78248 T88763 R93361 |
| 418546 | 176677_1 | AA224827 T59708 T59843 BE156903 |
| 418573 | 176907_1 | AA225188 AL157508 AL157509 AA225189 |
| 418636 | 177402_1 | AW749855 AA225995 AW750208 AW750206 |
| 418712 | 1784125_1 | Z42183 T31621 T97478 |
| 418948 | 180808_1 | AI217097 AW886090 W38035 W38792 AA232835 AW936043 |
| 419059 | 1816038_1 | T86216 T86117 T83316 |
| 419145 | 182217_1 | N99638 AW973750 AA328271 H90994 AA558020 AA234435 N59599 R94815 |
| 419472 | 185148_1 | AW978038 AA804204 AA243400 |
| 419477 | 185172_1 | AA826279 AA243426 AW971614 AW967805 AW971605 |
| 419716 | 187390_1 | AA953770 AA249091 |
| 419805 | 188248_1 | AW966945 AA250984 H91733 AW966941 |
| 419807 | 188252_1 | R77402 AA262462 AA250988 R06794 |
| 419834 | 188386_1 | AA251139 AW967485 AA251204 |
| 419932 | 189159_1 | AA281594 AI365043 AA251996 |
| 419998 | 189768_1 | AA252691 AW298356 AA688063 |
| 420111 | 190755_1 | AA255652 AA280911 AW967920 AA262684 |
| 420270 | 192170_1 | AA257990 AW816460 AI416981 AW500873 |
| 420643 | 195293_1 | W87731 AA279004 AA279001 |
| 420928 | 197723_1 | AA281809 H89487 N46537 |
| 421280 | 200885_1 | AA811804 AA809404 AA286907 AW977624 |
| 421338 | 201378_1 | AA287443 AA419385 BE084078 AI478347 |
| 421381 | 201903_1 | AA361752 AW963276 AA288017 |
| 421418 | 202288_1 | AA806639 AA291008 AA836274 AW978806 |
| 421708 | 205732_1 | AW754341 AW858420 AW858475 AW861969 AW861925 AW858554 AW858616 AW858450 AW858545 |
| 421813 | 207654_1 | BE048255 AA313083 AA298419 |
| 422204 | 213258_1 | AA339015 AW962948 AA306188 |
| 422342 | 215498_1 | AA309272 AA309312 AW961837 |
| 422429 | 216469_1 | AA310527 AW962295 Z44865 H06641 |
| 422504 | 217160_1 | AA311407 AW958321 N23583 R70050 |
| 422588 | 218192_1 | AA312730 AW963285 W28250 W27318 |
| 422760 | 221034_1 | BE409561 BE162756 AW732798 |
| 422834 | 221906_1 | AA318334 AW961457 AA317752 |
| 423100 | 224988_1 | AA323114 AA321992 BE161391 BE161392 |
| 423121 | 225175_1 | AW864848 AA322213 AA322209 AW961624 |
| 423368 | 227560_1 | AA364195 AA325029 AW962050 |
| 423632 | 230430_1 | AA328824 AW961410 AW958096 |
| 423644 | 230585_1 | AA329048 AW962484 T83628 |
| 423818 | 232362_1 | AA332439 AA332579 AA331470 AW962539 |
| 423837 | 232478_1 | AW937063 AW937056 AW937062 AW937059 AW937057 AA331599 AW937061 AW937093 AW937094 AW937058 |
| | | AW851327 BE082872 AW752885 AW937055 AW937054 |
| 423912 | 233241_1 | BE091233 BE080904 BE079414 BE075356 AA332482 |
| 424353 | 238398_1 | AA339646 AA339189 AW966286 |
| 424589 | 241202_1 | AW854298 AA343691 H50917 H50907 H50938 |
| 424951 | 245273_1 | AW964082 AA348838 AA348839 |
| 424993 | 245782_1 | F07625 R35115 AW953115 F06102 H13351 AA349497 F11152 AA707254 |
| 425360 | 250631_1 | BE547704 AA355909 |
| 425362 | 250655_1 | AA355936 AI741379 |
| 425672 | 254716_1 | AA361483 AI557239 |
| 426356 | 265381_1 | BE536836 AA376153 |
| 426475 | 267785_1 | AL134728 AA379975 AA379440 AW963931 |
| 426561 | 269158_1 | AA381437 AA628833 AW407275 |
| 426603 | 269825_1 | AA382291 AA994657 |
| 427259 | 276594_1 | AA400096 AA400167 |
| 427566 | 280401_1 | AI743515 AA405617 AW276706 |
| 427980 | 285225_1 | AA418305 AI264351 |
| 428042 | 286292_1 | AA419529 H97089 H96977 |
| 428192 | 288021_1 | AA424051 AW104616 |
| 428337 | 289967_1 | AA644508 AA479489 AA426174 |
| 428436 | 291472_1 | BE080180 AW827313 AW231970 AA995028 AA428584 AW872716 AW892508 AW854593 AA578441 |
| | | AW975234 AA664937 AA984131 AA528743 AA552874 AA564758 AW063245 AI267534 AW070190 AW893483 AA770330 |
| | | AA906928 AA906582 AA758746 AA551717 AW063311 AA429538 |
| 429236 | 301580_1 | AA448407 AA448404 AA448424 AA448426 |
| 429917 | 31049_1 | H80572 AL109691 H80520 |
| 429979 | 311517_1 | AA463338 M86137 M85945 AI040226 |
| 430076 | 312952_1 | AA465115 AW967750 AW869525 |
| 430350 | 316401_1 | BE169639 AA476976 |
| 430561 | 319932_1 | BE065227 BE065366 BE065182 BE065184 AA481239 |
| 430640 | 321094_1 | AA482636 R16336 R16343 R60706 R53647 |
| 430795 | 323756_1 | AW971398 AA507162 AA486494 |
| 431319 | 331474_1 | AA873350 T82429 T82428 |
| 431322 | 331543_1 | AW970622 AA503009 AA502998 AA502989 AA502805 T92188 |
| 431670 | 336353_1 | AW971287 AA524976 AA513479 |
| 431828 | 338201_1 | AA572994 AA516249 AA702595 |
| 432093 | 341283_1 | H28383 AW972670 H28359 AA525808 |
| 432222 | 343347_1 | AI204995 AW827539 AW969908 AW440776 AA528756 |
| 432724 | 35330_1 | X98266 N41124 |
| 433005 | 357346_1 | AW939074 AW939073 BE160476 AW939938 AW939206 AW940012 AW939076 AA573577 AW750479 |
| | | AA574383 AW970057 |
| 433108 | 35896_1 | AB002446 T03146 |
| 433129 | 359479_1 | AA577814 N68543 N68478 H79613 AI910764 |
| 433171 | 360292_1 | AA579425 AW969965 AA579102 |
| 433311 | 362908_1 | AA688149 AW975336 R82513 R79237 |
| 433371 | 364430_1 | T25451 AA585296 AA585305 |
| 433440 | 36629_−1 | AF052127 |
| 433586 | 370470_1 | T85301 AW517087 AA601054 BE073959 |
| 433782 | 37414_1 | AF090945 AW996754 AI064870 |
| 434006 | 37855_1 | AF113688 AI114617 |
| 434092 | 379978_1 | AA625155 BE502124 |
| 434138 | 380572_1 | AA625804 AW418787 AW074833 AI675642 AI393368 |
| 434280 | 382816_1 | BE005398 AA628622 AA994155 |
| 435382 | 405265_1 | N54493 AA679039 N76605 |
| 436023 | 413562_1 | T81819 AW393709 AA703541 AW370185 |
| 436359 | 41847_1 | Z83806 AJ132091 AJ132090 |
| 436383 | 41888_1 | BE065178 AJ227879 |
| 436740 | 426095_1 | AW975133 AA729943 AA805813 |
| 436884 | 428731_1 | BE046657 AA736610 AW827495 |
| 437087 | 432797_1 | AA745563 AA745334 AA744168 AA744044 AA744034 AA744056 AA745569 AA744397 |
| 437146 | 43371_1 | AA730977 AI261584 AA334473 Z43283 AW875861 AW938044 BE150701 AW936262 AA306862 |
| | | BE565575 BE567380 AA728920 AA167612 AI239729 AI251752 AA485791 BE568425 AW962958 |
| 437214 | 434730_1 | BE092336 BE092259 BE092497 BE092051 AA746882 AI336378 |
| 437240 | 435139_1 | AA747537 BE089068 BE089070 |
| 437251 | 435242_1 | AW976082 AW976085 AA747803 AA747680 |
| 437274 | 435465_1 | AA747965 AI248281 AI752458 |
| 437324 | 43609_1 | AI162077 AW023677 |
| 437471 | 43747_1 | AL390169 AW277196 AW131001 |
| 437798 | 443128_1 | AW811767 AA831890 AW977539 AA768655 |
| 437854 | 44418_1 | AL119723 AL119874 AI909018 U50537 |
| 437938 | 44573_2 | AI950087 N70208 R97040 N36809 AI308119 AW967677 N35320 AI251473 H59397 AW971573 R97278 W01059 |
| | | AW967671 AA908598 AA251875 AI820501 AI820532 W87891 T85904 U71456 T82391 BE328571 T75102 R34725 |
| | | AA884922 BE328517 AI219788 AA884444 N92578 F13493 AA927794 AI560251 AW874068 AL134043 AW235363 |
| | | AA663345 AW008282 AA488964 AA283144 AI890387 AI950344 AI741346 AI689062 AA282915 AW102898 AI872193 |
| | | AI763273 AW173586 AW150329 AI653832 AI762688 AA988777 AA488892 AI356394 AW103813 AI539642 AA642789 |
| | | AA856975 AW505512 AI961530 AW629970 BE612881 AW276997 AW513601 AW512843 AA044209 AW856538 |
| | | AA180009 AA337499 AW961101 AA251669 AA251874 AI819225 AW205862 AI683338 AI858509 AW276905 AI633006 |
| | | AA972584 AA908741 AW072629 AW513996 AA293273 AA969759 N75628 N22388 H84729 H60052 T92487 AI022058 |
| | | AA780419 AA551005 W80701 AW613456 AI373032 AI564269 F00531 H83488 W37181 W78802 R66056 AI002839 |
| | | R67840 AA300207 AW959581 T63226 F04005 |
| 438316 | 454745_1 | AA789249 AA904217 AA904142 |
| 438366 | 456119_1 | AA805760 BE000856 |
| 438390 | 45662_1 | AI422017 AI422945 AI363249 AI423113 AI925592 AI420795 AI208187 AI423279 AI423645 AI424090 AI359637 |
| | | AI044732 D17003 |
| 438739 | 464193_1 | AA815391 AW573185 |
| 438990 | 46760_1 | AF085890 H29949 H29856 |
| 439183 | 46956_1 | AW970600 AA503323 H89218 AF086031 H89112 |
| 439330 | 47119_1 | AF086147 W20068 N91464 |
| 439550 | 473671_1 | H10438 AA837479 H16299 AW978825 |
| 439780 | 47673_1 | AL109688 R23665 R26578 |
| 440012 | 483290_1 | AA861072 H02819 R25946 |
| 440635 | 498968_1 | AW610331 AW610338 AA897379 |
| 442048 | 531432_1 | AA974603 AI984319 AW340495 |
| 442518 | 544159_1 | AF150226 AI240137 |
| 444163 | 593658_1 | AI126098 AI184746 AI148521 |
| 444282 | 599268_1 | AI138955 BE149059 BE149027 |
| 444584 | 611496_1 | AI168422 D80113 T59074 |
| 444646 | 613548_1 | AI184565 AL037304 AW793549 |
| 445396 | 638138_1 | BE181792 AI222266 BE181789 BE181837 |
| 445832 | 651925_1 | AI261545 N59134 AW875371 AW875247 |
| 446119 | 662531_1 | D29527 AI275850 |
| 446312 | 671114_1 | BE087853 AI286184 |
| 446922 | 69865_1 | BE175605 Z43529 F06610 BE175602 AV661027 |
| 447258 | 71439_1 | BE047911 AA984167 BE617316 |
| 447376 | 719483_−1 | AI376747 |
| 447522 | 724922_1 | BE143888 AI382949 T06429 |
| 448632 | 77303_−1 | BE614269 |
| 448643 | 773566_−1 | AI557531 |
| 448778 | 7800_1 | AF074913 AW505435 U62539 |
| 448871 | 78566_2 | BE616709 BE616712 |
| 449213 | 80240_−1 | BE616861 |
| 449231 | 80303_1 | BE410360 AA442408 AA315540 |
| 449344 | 805184_1 | AI640355 BE464385 AW197679 |
| 449666 | 81249_1 | AA002047 N72537 H54142 H81580 |
| 449901 | 818599_1 | AI674072 BE268487 |
| 450257 | 830121_1 | AW820313 BE065936 BE065948 BE155658 AI689473 AW862275 AW820265 AW820438 AW820331 |
| 450573 | 83907_1 | AW964334 AW967230 T08913 R11895 F11463 AA010271 |
| 450625 | 84032_1 | AW970107 AA513951 AA010406 |
| 450870 | 84994_1 | AA011471 AA700636 |
| 450928 | 851593_1 | AI744417 R91614 H77365 |
| 451283 | 86479_1 | H83979 R84433 AA017024 |
| 451359 | 86711_1 | H85334 AA017286 H86654 |
| 451365 | 867325_−1 | AI791783 |
| 451373 | 867533_1 | AI792030 AW386116 AW875146 |
| 451487 | 87131_1 | AA018072 N46370 R84847 |
| 451495 | 87182_1 | H86887 H86898 H86906 AA019519 AA018127 AA056306 AA053726 |
| 451535 | 87353_1 | AW970577 AA861861 AA418358 AA018329 AA501885 |
| 451724 | 882130_1 | AI903765 AI811194 BE007147 AW130760 |
| 451844 | 888230_1 | T61430 AI820546 AI821336 |
| 452163 | 902067_1 | AI863140 W80703 R43474 |
| 452453 | 918300_1 | AI902519 AI902518 AI902516 |
| 452457 | 918409_1 | AW062499 AW062495 AW062500 AW062492 AW062494 AW062493 AW062496 AW062498 AI902650 AI902649 |
| 452519 | 920292_1 | BE006701 BE006709 BE006704 AI904981 |
| 452530 | 920646_1 | AI905518 AI905516 AI905457 AI905515 AW176013 AW176037 |
| 452536 | 921149_1 | BE063380 BE063346 AI906097 |
| 452550 | 92188_1 | AA026735 BE264405 AL040323 AA323099 |
| 452560 | 922216_1 | BE077084 AW139963 AW863127 AW806209 AW806204 AW806205 AW806206 AW806211 AW806212 |
| | | AW806207 AW806208 AW806210 AI907497 |
| 452738 | 929698_1 | AL133800 AI916209 |
| 452825 | 933090_1 | AI921523 AW903707 AW903687 |
| 453472 | 968371_1 | AL037925 AL037931 AL037957 |
| 453677 | 977149_1 | AL079389 AL079390 |
| 453725 | 978760_1 | W28543 AL119531 |
| 453846 | 983043_1 | AL157586 AL157590 |
| 454044 | 996486_1 | AW022393 AW022578 |
| 454049 | 996858_1 | AW022885 AW023064 |
| 454099 | 1007650_1 | AW062974 AW859625 BE081366 |
| 454314 | 1108161_1 | AW364844 AW364847 AW937534 AW937593 AW937659 |
| 454327 | 1114097_1 | BE064097 BE064124 BE064087 AW371617 |
| 454331 | 1115278_1 | AW372937 AW378043 AW377970 BE145850 AW377858 AW377964 AW378040 |
| 454380 | 1152648_−1 | AW858722 |
| 454471 | 1213795_1 | AW902125 AW792853 AW837703 AW837710 |
| 454524 | 1221640_1 | AW857191 AW857192 AW804060 |
| 454554 | 1223842_1 | AW847505 AW811792 BE061442 BE061433 AW847506 AW806999 AW806996 BE061436 BE061430 BE142460 BE146499 |
| | | AW806994 AW809156 AW806991 AW814082 AW806992 BE061669 AW807002 BE146659 AW806995 AW807000 |
| | | AW845743 AW845747 AW847504 BE142458 BE061431 BE061435 AW847507 BE146650 BE142470 AW814096 |
| | | AW807012 BE061438 AW807011 AW806993 BE142465 BE142459 BE142462 AW854330 AW854331 BE061434 |
| | | BE061731 BE142464 AW847501 AW807001 BE142463 AW811800 BE061437 AW811802 BE061440 |
| | | AW806997 AW806998 BE061745 BE061753 |
| 454592 | 1226050_1 | AW810112 AW810114 AW810032 AW809631 AW810183 AW810178 |
| 454622 | 1226951_1 | U70071 AW883715 AW810701 |
| 454637 | 1227707_1 | AW811613 AW811611 AW811610 |
| 454648 | 1227976_1 | AW811960 AW811957 AW811959 |
| 454687 | 1229077_1 | AW814473 AW816157 AW816064 AW813298 AW813315 AW816100 AW813389 AW813338 AW813342 |
| | | AW816108 AW813318 AW816105 AW816160 AW816163 AW814480 AW813392 AW816063 AW813399 |
| 454692 | 1229118_1 | AW813350 AW816082 AW813476 AW813383 |
| 454702 | 1229722_1 | BE145915 BE145893 AW892892 BE145806 BE145901 AW814144 |
| 454729 | 1231926_1 | AW817003 AW816997 AW817126 AW817331 AW817120 BE158378 AW817121 |
| 454750 | 1233434_1 | AW866285 AW819000 AW819153 AW819014 AW866541 |
| 454775 | 1234106_1 | BE160229 AW819879 AW820179 AW819882 AW819876 AW820169 BE153201 AW993736 BE152911 |
| 454789 | 1234742_1 | BE156314 BE156316 AW820750 |
| 454790 | 1234752_1 | AW820852 AW820773 AW821088 |
| 454797 | 1235093_1 | BE161168 BE162466 AW821260 |
| 454851 | 1237414_1 | AW835127 BE071463 AW991660 BE071473 BE071461 BE071469 BE071472 |
| 454863 | 1237794_1 | AW835610 AW835806 |
| 454893 | 1239472_1 | AW837753 AW837754 AW837700 |
| 454898 | 1239833_1 | AW838125 AW838205 |
| 454951 | 1246612_1 | AW847464 AW847462 BE063767 BE063755 |
| 454956 | 1246711_1 | AW847725 AW847599 AW847600 AW847706 AW847854 AW847595 AW847850 AW847795 AW847798 |
| 455040 | 1250028_1 | AW852286 AW851934 AW852096 AW852274 |
| 455047 | 1250536_1 | AW852530 AW852527 AW852526 |
| 455073 | 1252361_1 | AW854829 AW854805 AW854841 AW854825 AW854822 AW854830 AW854835 AW854826 |
| 455128 | 1254554_1 | AW861555 AW857776 AW861556 AW861567 AW857799 AW857768 |
| 455170 | 1256906_1 | AW860972 AW862598 AW862599 AW860988 AW860983 AW860898 AW860925 AW860922 AW860986 AW860984 |
| | | AW860989 |
| 455201 | 1259748_1 | AW947884 AW947918 AW947888 AW947883 AW947897 AW947910 AW947905 AW864751 AW947878 |
| 455207 | 1260400_1 | AW994394 AW865900 AW865905 AW865891 AW866014 AW865898 |
| 455225 | 1262318_1 | AW996689 AW996380 AW996453 BE085650 AW868687 BE085595 |
| 455235 | 1265634_1 | AW875951 AW875950 AW875936 AW875948 AW875939 AW875957 |
| 455267 | 1269025_1 | AW880861 AW880883 AW881117 AW881012 |
| 455331 | 1280616_1 | AW897292 AW897251 AW897298 AW897248 AW897259 AW897250 AW897246 AW897242 |
| 455350 | 1283853_1 | AW901809 AW901787 AW901795 AW901792 AW901744 AW901753 AW901807 AW901798 |
| 455351 | 1284023_1 | AW901942 AW901937 AW902016 AW901947 |
| 455380 | 1287679_1 | BE160188 AW935785 BE160401 BE160319 BE160313 BE160395 |
| 455414 | 1288605_1 | AW936969 AW936920 AW936975 AW936906 |
| 455428 | 1289639_1 | AW938204 AW938195 AW938205 AW938206 AW938193 AW938199 AW938200 AW938190 AW938201 |
| | | AW938198 AW938203 AW938202 AW938192 AW938207 AW938194 |
| 455511 | 1321229_1 | BE144762 AW979091 |
| 455573 | 1332529_1 | BE004988 BE004574 |
| 455586 | 1334857_1 | BE070794 BE070791 BE070792 BE070795 BE070789 BE070788 BE070786 BE070787 BE070790 |
| 455595 | 1335422_1 | BE008343 BE008321 BE008326 BE008314 BE008345 BE008330 BE008344 BE008327 |
| 455610 | 1337549_1 | BE011703 BE011692 BE011697 BE011624 BE011673 BE011644 |
| 455647 | 1348563_1 | BE064415 BE064430 BE064448 |
| 455650 | 1348720_1 | BE064655 BE153953 |
| 455657 | 1349078_1 | BE065209 BE065364 BE065110 BE065111 |
| 455669 | 1349554_1 | BE065803 BE065826 |
| 455678 | 1349716_1 | BE066007 BE066017 BE066074 |
| 455761 | 1359566_1 | BE080895 BE080906 |
| 455831 | 1373969_1 | BE144966 BE144957 BE144958 |
| 455874 | 1379514_1 | BE152283 BE152260 BE152297 BE152292 BE152259 BE152250 |
| 455880 | 1380022_1 | BE153208 BE153146 BE152981 |
| 455903 | 1381603_1 | BE155185 BE155264 BE155186 |
| 455938 | 1384537_1 | BE159432 BE159313 BE159295 BE159242 |
| 455950 | 1385884_1 | BE161004 BE162497 BE161135 BE160999 BE162492 |
| 455951 | 1385886_1 | BE161001 BE162494 BE162470 BE161172 |
| 455965 | 1389984_1 | BE167014 BE167058 BE167062 |
| 455981 | 1396805_1 | BE177000 BE177003 BE176816 BE176952 |
| 456034 | 142696_1 | AW450979 AA136653 AA136656 AW419381 AA984358 AA492073 BE168945 AA809054 AW238038 BE011212 |
| | | BE011359 BE011367 BE011368 BE011362 BE011215 BE011365 BE011363 |
| 456122 | 153360_1 | R11813 AA164376 T56632 |
| 456212 | 1655565_1 | N51636 T51874 T51829 |
| 456235 | 168686_1 | AA203637 AA832266 H67452 |
| 456493 | 193331_1 | AA261830 AW967855 H26953 AA262478 |
| 456592 | 202684_1 | R91600 T87079 AA291455 |
| 456800 | 233842_1 | AL118754 AA333202 H38001 |
| 457340 | 322100_1 | AA492071 AA484229 AW969850 |
| 457427 | 336353_1 | AW971287 AA524976 AA513479 |
| 457437 | 337879_1 | AW969732 AW970974 AA515741 |
| 457474 | 341077_1 | AW972935 AA525272 N28227 |
| 457625 | 373012_1 | T10073 H14872 AA604786 |
| 457892 | 432926_1 | AA744389 AA744270 AA744284 AA744299 AA745380 AA744337 AA846905 AA847698 |
| 458080 | 471050_1 | BE142728 AA834047 AW937124 |
| 458172 | 497769_1 | BE007237 BE007496 BE007263 W19919 BE546311 AA984819 |
| 458781 | 743159_1 | AI444821 W26012 |
| 458841 | 784186_1 | W28965 W28971 |
| 458880 | 80785_1 | AA046742 AA551269 AA001505 |
| 459028 | 868710_1 | AI940577 AI940580 AI940568 AI940578 AI940569 AI795858 AI795867 AI940572 |
| 459128 | 918011_1 | AI902169 AI902173 AI902323 AI902174 AI902347 |
| 459182 | 922744_1 | BE178517 AI908132 BE142437 |

Pkey: Unique Eos probeset identifier number
CAT number: Gene cluster number
Accession: Genbank accession numbers

TABLE 1C

| Pkey | Ref | Strand | Nt_position |
|---|---|---|---|
| 400471 | 9931670 | Minus | 105629-105760 |
| 400495 | 9714773 | Minus | 61902-62137 |
| 400528 | 6981824 | Plus | 472381-472528, 474170-474277, 475328-475542, 475878-476000 |
| 400577 | 7960401 | Plus | 101535-101881 |
| 400608 | 9887666 | Minus | 96756-97558 |
| 400641 | 8117693 | Plus | 4786-4992 |
| 400644 | 8117693 | Plus | 27682-27840 |
| 400666 | 8118496 | Plus | 17982-18115, 20297-20456 |
| 400749 | 7331445 | Minus | 9162-9293 |
| 400750 | 8119067 | Plus | 198991-199168, 199316-199548 |
| 400751 | 7331445 | Minus | 35395-35533 |
| 400761 | 8131609 | Minus | 114220-115164 |
| 400762 | 8131616 | Plus | 7235-7605 |
| 400773 | 8131629 | Minus | 44116-44238, 48208-48321 |
| 400843 | 9188605 | Plus | 5863-5970, 7653-7784, 8892-9023, 9673-9807, 10634-10789, 15254-15403, 23827-23958 |
| 400844 | 9188605 | Plus | 24746-24872, 25035-25204 |
| 400845 | 9188605 | Plus | 34428-34612 |
| 400846 | 9188605 | Plus | 39310-39474 |
| 400860 | 9757499 | Minus | 151830-152104, 152649-152744 |
| 400880 | 9931121 | Plus | 29235-29336, 36363-36580 |
| 400887 | 9958187 | Plus | 119239-121542 |
| 400888 | 9958187 | Minus | 199600-199875 |
| 400937 | 7652890 | Minus | 89519-89905 |
| 400977 | 8072510 | Plus | 73950-74364 |
| 401002 | 8117251 | Minus | 77898-78050 |
| 401024 | 8117489 | Plus | 60551-60802 |
| 401045 | 8117619 | Plus | 90044-90184, 91111-91345 |
| 401048 | 7232177 | Plus | 132430-132761 |
| 401049 | 7232177 | Plus | 149157-150692 |
| 401086 | 9957912 | Plus | 84561-84884 |
| 401093 | 8516137 | Minus | 22335-23166 |
| 401101 | 8568122 | Plus | 77081-77226 |
| 401192 | 9719502 | Minus | 69559-70101 |
| 401197 | 9719705 | Plus | 176341-176452 |
| 401203 | 9743387 | Minus | 172961-173056, 173868-173928 |
| 401205 | 9743388 | Plus | 167373-167433, 167936-168031 |
| 401256 | 9796573 | Minus | 45482-45620 |
| 401262 | 9796963 | Plus | 35662-35799 |
| 401271 | 9797373 | Minus | 61292-61911 |
| 401276 | 8954274 | Minus | 15919-16096 |
| 401279 | 9800062 | Minus | 13535-13669 |
| 401342 | 9908882 | Plus | 3096-3242 |
| 401365 | 9796180 | Minus | 119572-119672 |
| 401395 | 7327842 | Minus | 11502-11771, 46357-46489, 58720-58916 |
| 401420 | 7452889 | Minus | 141362-141502 |
| 401439 | 8246737 | Plus | 92993-94026 |
| 401451 | 6634068 | Minus | 119926-121272 |
| 401508 | 7534110 | Minus | 110779-110983 |
| 401519 | 6649315 | Plus | 157315-157950 |
| 401537 | 7960358 | Minus | 186786-187029, 190607-190779, 198218-198348 |
| 401561 | 8224660 | Minus | 10652-10838, 19815-20018 |
| 401566 | 8469090 | Minus | 96277-96420, 96979-97160 |
| 401604 | 7689963 | Minus | 119835-120185 |
| 401619 | 8516761 | Minus | 141309-143576 |
| 401669 | 9801805 | Plus | 25414-26310 |
| 401691 | 3582311 | Plus | 162333-162715 |
| 401694 | 3540172 | Minus | 64056-64168 |
| 401747 | 9789672 | Minus | 118596-118816, 119119-119244, 119609-119761, 120422-120990, 130161-130381, 130468-130593, |
| | | | 131097-131258, 131866-131932, 132451-132575, 133580-134011 |
| 401759 | 9929699 | Plus | 59811-60665 |
| 401780 | 7249190 | Minus | 28397-28617, 28920-29045, 29135-29296, 29411-29567, 29705-29787, 30224-30573 |
| 401866 | 8018106 | Plus | 73126-73623 |
| 401905 | 8671966 | Plus | 153965-154441, 156599-156819 |
| 401994 | 4153858 | Minus | 42904-43124, 43211-43336, 44607-44763, 45199-45281, 46337-46732 |
| 402001 | 9501818 | Plus | 68052-68223 |
| 402075 | 8117407 | Plus | 121907-122035, 122804-122921, 124019-124161, 124455-124610, 125672-126076 |
| 402076 | 8117410 | Plus | 128316-128627 |
| 402089 | 7249154 | Plus | 101610-101819 |
| 402110 | 8131678 | Minus | 173889-174062 |
| 402176 | 7543687 | Minus | 10-750 |
| 402230 | 9966312 | Minus | 29782-29932 |
| 402245 | 7690231 | Minus | 88253-88417 |
| 402296 | 6598824 | Plus | 22587-23723 |
| 402325 | 7636348 | Minus | 60658-60738, 61677-61803 |
| 402407 | 3962498 | Minus | 115812-116187 |
| 402408 | 9796239 | Minus | 110326-110491 |
| 402430 | 9796372 | Minus | 62382-62552 |
| 402435 | 9796462 | Plus | 114593-115588 |
| 402472 | 9797116 | Plus | 53716-54470 |
| 402474 | 7547175 | Minus | 53526-53628, 55755-55920, 57530-57757 |
| 402480 | 9797375 | Plus | 59708-59999 |
| 402490 | 9797648 | Plus | 149982-150929 |
| 402522 | 9798493 | Plus | 20605-20731 |
| 402530 | 7630937 | Minus | 1524-2003 |
| 402546 | 7637348 | Plus | 24673-25170 |
| 402553 | 9863566 | Plus | 48292-48398, 49564-49944 |
| 402604 | 9909420 | Plus | 20393-20767 |
| 402716 | 8969253 | Minus | 84065-84242 |
| 402727 | 9211324 | Plus | 54596-54777 |
| 402812 | 6010110 | Plus | 25026-25091, 25844-25920 |
| 402820 | 6456853 | Minus | 82274-82443 |
| 402846 | 9408716 | Minus | 5726-5850 |
| 402889 | 9931133 | Plus | 89392-89498, 90358-90571 |
| 402892 | 8086844 | Minus | 194384-194645 |
| 402901 | 8894222 | Minus | 175426-175667 |
| 402922 | 8216969 | Minus | 19036-19401, 19589-19849, 19951-20102 |
| 402938 | 8953442 | Plus | 22365-22473 |
| 402995 | 2996643 | Minus | 5962-6216 |
| 403005 | 5791501 | Minus | 16945-17053, 20018-20403 |
| 403020 | 6984114 | Minus | 96644-97021, 97462-97868 |
| 403029 | 7768593 | Minus | 44558-44766 |
| 403047 | 3540153 | Minus | 59793-59968 |
| 403073 | 8954241 | Plus | 142964-143260 |
| 403085 | 8954241 | Plus | 165035-165334, 165420-165713 |
| 403092 | 8954241 | Plus | 174720-175016, 175104-175406, 175508-175813 |
| 403106 | 7331404 | Plus | 77162-77350, 81338-81511 |
| 403172 | 7464784 | Minus | 64007-64275 |
| 403212 | 7630897 | Minus | 156037-156210 |
| 403214 | 7630945 | Minus | 76723-77027, 79317-79484 |
| 403277 | 8072597 | Minus | 27494-27642 |
| 403331 | 8567936 | Plus | 169793-169966 |
| 403344 | 8569726 | Plus | 70823-70990 |
| 403362 | 8571772 | Plus | 64099-64260 |
| 403381 | 9438267 | Minus | 26009-26178 |
| 403426 | 9719529 | Minus | 157156-158183 |
| 403485 | 9966528 | Plus | 2888-3001, 3198-3532, 3655-4117 |
| 403567 | 8101141 | Plus | 35349-35614 |
| 403588 | 8101227 | Minus | 197672-197944 |
| 403590 | 8101229 | Plus | 405-1296 |
| 403615 | 8567964 | Plus | 107671-107866 |
| 403687 | 7387384 | Plus | 9009-9534 |
| 403754 | 7229815 | Minus | 163899-164726 |
| 403776 | 7770611 | Minus | 1414-1513, 1624-1756 |
| 403822 | 9369510 | Minus | 142803-142922 |
| 403851 | 7708872 | Plus | 22733-23007 |
| 403860 | 7708960 | Minus | 95755-96045 |
| 403894 | 7381715 | Minus | 1442-2224 |
| 403903 | 7710671 | Minus | 101165-102597 |
| 403959 | 8224399 | Minus | 175363-177474 |
| 404015 | 8655948 | Minus | 587821-588222 |
| 404059 | 3548785 | Plus | 104326-106788 |
| 404113 | 9588571 | Minus | 13446-13646 |
| 404148 | 9863703 | Plus | 78218-78418, 79571-79709 |
| 404152 | 9884757 | Plus | 41111-41281, 45495-45716, 47801-47910 |
| 404156 | 9886577 | Plus | 127319-127754 |
| 404229 | 7159766 | Plus | 16607-16841 |
| 404232 | 8218045 | Minus | 71800-71956 |
| 404268 | 9711362 | Plus | 33238-33463 |
| 404274 | 9885189 | Plus | 104127-104318 |
| 404288 | 2769644 | Plus | 3512-3691 |
| 404290 | 2769644 | Plus | 36651-36813 |
| 404336 | 9838028 | Plus | 157951-158129 |
| 404403 | 7272157 | Minus | 72053-72238 |
| 404440 | 7528051 | Plus | 80430-81581 |
| 404488 | 8113286 | Minus | 64835-64994 |
| 404498 | 8151654 | Plus | 13292-13497 |
| 404507 | 8151803 | Plus | 146359-146739 |
| 404516 | 8151967 | Plus | 114153-114322 |
| 404538 | 8247909 | Minus | 192748-192945 |
| 404594 | 9958262 | Minus | 15310-15510 |
| 404639 | 9796778 | Plus | 5779-14387 |
| 404653 | 9796999 | Plus | 164997-165230 |
| 404676 | 9797204 | Minus | 56167-56342, 58066-58189, 58891-59048, 60452-60628 |
| 404684 | 9797403 | Minus | 110881-111020 |
| 404685 | 9797437 | Minus | 153217-153315, 154043-154124, 159185-159353, 161290-161420, 163544-163669, 166127-166207, 167654-167734 |
| 404704 | 9800728 | Minus | 88841-89018 |
| 404819 | 4678240 | Plus | 16223-16319, 16427-16513, 16736-16859, 16941-17075, 17170-17287, 17389-17529, 18261-18357, 18443-18578 |
| 404829 | 6624702 | Minus | 4913-5093, 7310-7469, 9472-9621, 9951-10082 |
| 404860 | 8979555 | Plus | 65852-66081 |
| 404874 | 9650523 | Minus | 96066-96192 |
| 404881 | 5931510 | Minus | 36360-36608 |
| 404894 | 6850447 | Plus | 102822-103127 |
| 404939 | 6862697 | Plus | 175318-175476 |
| 404977 | 3738341 | Minus | 43081-43229 |
| 405033 | 7107731 | Minus | 142358-142546 |
| 405059 | 7656683 | Plus | 349-822 |
| 405064 | 7658416 | Plus | 81207-81416 |
| 405071 | 7708797 | Minus | 11115-11552 |
| 405102 | 8076881 | Minus | 120922-121296 |
| 405167 | 9966316 | Plus | 43796-43981, 48245-48427, 54141-54317 |
| 405170 | 9966524 | Plus | 37047-37198 |
| 405177 | 7139696 | Minus | 118466-118663 |
| 405186 | 7229793 | Plus | 161475-161581, 162930-163067 |
| 405258 | 7329310 | Plus | 129930-130076 |
| 405281 | 6139075 | Minus | 34202-34351, 35194-35336, 45412-45475, 45731-45958, 47296-47457, 49549-49658, 49790-49904, |
| | | | 50231-50342, 53583-53667, 54111-54279 |
| 405308 | 3638954 | Plus | 40778-41034, 41383-41573 |
| 405349 | 2914717 | Minus | 85552-85806 |
| 405379 | 6513908 | Minus | 22332-22473, 24333-24439 |
| 405390 | 6606064 | Minus | 94007-94177 |
| 405411 | 3451356 | Minus | 17503-17778, 18021-18290 |
| 405463 | 7715630 | Minus | 123097-123260 |
| 405494 | 8050952 | Minus | 70284-70518 |
| 405520 | 9454643 | Plus | 60849-60981 |
| 405526 | 9558556 | Minus | 132704-133277 |
| 405580 | 4512267 | Plus | 169232-169647 |
| 405600 | 5923640 | Plus | 26662-27225 |
| 405654 | 4895155 | Minus | 53624-53759 |
| 405720 | 9797144 | Plus | 13409-13861 |
| 405725 | 9838299 | Minus | 106417-106521 |
| 405735 | 9931101 | Minus | 29854-29976 |
| 405738 | 9943998 | Plus | 44370-45410 |
| 405809 | 5304920 | Minus | 6655-6883, 8687-8859 |
| 405838 | 5686575 | Plus | 3460-3717 |
| 405863 | 7657810 | Plus | 49410-49620 |
| 405867 | 6758731 | Minus | 74553-75173 |
| 405906 | 7705124 | Minus | 10835-11059 |
| 405920 | 6758795 | Plus | 120621-120971 |
| 405968 | 8247789 | Plus | 14893-15148 |
| 406017 | 8272661 | Minus | 46271-46874 |
| 406036 | 6758919 | Plus | 17942-18163 |
| 406081 | 9123861 | Minus | 38115-38691 |
| 406137 | 9166422 | Minus | 30487-31058 |
| 406187 | 7289992 | Plus | 8044-8877 |
| 406243 | 7417725 | Plus | 38899-39369 |
| 406270 | 7534217 | Plus | 13136-13591 |
| 406320 | 9211754 | Minus | 20170-20511 |
| 406322 | 9212102 | Minus | 130230-130418 |
| 406360 | 9256107 | Minus | 7513-7673 |
| 406367 | 9256126 | Minus | 58313-58489 |
| 406397 | 9256243 | Minus | 127317-127454 |
| 406399 | 9256288 | Minus | 63448-63554 |
| 406434 | 9256651 | Minus | 17803-17931 |
| 406467 | 9795551 | Plus | 182212-182958 |
| 406471 | 9795566 | Plus | 87383-87589 |
| 406475 | 9797684 | Plus | 125417-125563, 128052-128180 |
| 406485 | 7711305 | Plus | 125036-125422 |
| 406511 | 7711412 | Plus | 177277-177384 |
| 406588 | 8189273 | Minus | 135629-135848 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt position: Indicates nucleotide positions of predicted exons.

TABLE 2A
485 GENES UP-REGULATED IN BLADDER CANCER

| Pkey | ExAccn | UnigeneID | Unigene Title | R1 |
|---|---|---|---|---|
| 418818 | AA228899 | Hs.101307 | Homo sapiens HUT11 protein mRNA, partial | 3.473 |
| 412841 | AI751157 | Hs.101395 | hypothetical protein MGC11352 | 2.279 |
| 421066 | AU076725 | Hs.101408 | branched chain aminotransferase 2, mitoc | 3.052 |
| 435136 | R27299 | Hs.10172 | ESTs | 4.717 |
| 444042 | NM_004915 | Hs.10237 | ATP-binding cassette, sub-family G (WHIT | 0.003 |
| 421307 | BE539976 | Hs.103305 | Homo sapiens mRNA; cDNA DKFZp434B0425 (f | 188.231 |
| 421318 | U63973 | Hs.103501 | rhodopsin kinase | 1.381 |
| 421359 | AK001589 | Hs.103816 | hypothetical protein FLJ10727 | 1.000 |
| 459462 | AA481396 | Hs.105167 | ESTs | 1.000 |
| 430134 | BE380149 | Hs.105223 | ESTs, Weakly similar to T33188 hypotheti | 1.000 |
| 433227 | AB040923 | Hs.106808 | kelch (Drosophila)-like 1 | 1.000 |
| 421742 | AW970004 | Hs.107528 | androgen induced protein | 1.514 |
| 417366 | BE185289 | Hs.1076 | small proline-rich protein 1B (cornifin) | 2.782 |
| 444342 | NM_014398 | Hs.10887 | similar to lysosome-associated membrane | 20.064 |
| 451686 | AA059246 | Hs.110293 | ESTs | 0.033 |
| 454417 | AI244459 | Hs.110826 | trinucleotide repeat containing 9 | 56.751 |
| 458760 | AI498631 | Hs.111334 | ferritin, light polypeptide | 2.512 |
| 422119 | AI277829 | Hs.111862 | KIAA0590 gene product | 2.634 |
| 422170 | AI791949 | Hs.112432 | anti-Mullerian hormone | 0.055 |
| 441877 | AW273802 | Hs.11340 | hypothetical protein FLJ23047 | 0.008 |
| 445958 | BE326257 | Hs.114536 | ESTs | 0.002 |
| 434288 | AW189075 | Hs.116265 | fibrillin3 | 11.401 |
| 435347 | AW014873 | Hs.116963 | ESTs | 0.003 |
| 453134 | AA032211 | Hs.118493 | ESTs | 262.962 |
| 444781 | NM_014400 | Hs.11950 | GPI-anchored metastasis-associated prote | 5.336 |
| 436154 | AA764950 | Hs.119898 | ESTs | 103.154 |
| 436246 | AW450963 | Hs.119991 | ESTs | 0.071 |
| 436293 | AI601188 | Hs.120910 | ESTs | 29.129 |
| 433078 | AW015188 | Hs.121575 | Homo sapiens cDNA FLJ12231 fis, clone MA | 274.769 |
| 438181 | AW978608 | Hs.122121 | ESTs, Weakly similar to I38022 hypotheti | 0.024 |
| 449399 | AA760881 | Hs.122408 | ESTs | 1.000 |
| 437722 | AW292947 | Hs.122872 | ESTs, Weakly similar to JU0033 hypotheti | 4.314 |
| 457465 | AW301344 | Hs.122908 | DNA replication factor | 0.264 |
| 409757 | NM_001898 | Hs.123114 | cystatin SN | 1.390 |
| 439907 | AA853978 | Hs.124577 | ESTs | 0.010 |
| 437181 | AI306615 | Hs.125343 | ESTs, Weakly similar to KIAA0758 protein | 0.344 |
| 440304 | BE159984 | Hs.125395 | ESTs | 0.025 |
| 423248 | AA380177 | Hs.125845 | ribulose-5-phosphate-3-epimerase | 0.014 |
| 441495 | AW294603 | Hs.127039 | ESTs | 0.198 |
| 435376 | AW770956 | Hs.127280 | ESTs | 0.008 |
| 427685 | AI751124 | Hs.127311 | ESTs | 3.244 |
| 423349 | AF010258 | Hs.127428 | homeo box A9 | 0.134 |
| 445457 | AF168793 | Hs.12743 | carnitine O-octanoyltransferase | 7.255 |
| 420759 | T11832 | Hs.127797 | Homo sapiens cDNA FLJ11381 fis, clone HE | 1.000 |
| 441875 | AI435973 | Hs.128056 | ESTs | 0.013 |
| 441940 | AW298115 | Hs.128152 | ESTs | 6.075 |
| 445537 | AJ245671 | Hs.12844 | EGF-like-domain, multiple 6 | 0.137 |
| 429983 | W92620 | Hs.128656 | ESTs | 162.590 |
| 445600 | AF034803 | Hs.12953 | PTPRF interacting protein, binding prote | 0.969 |
| 437553 | AI829935 | Hs.130497 | ESTs, Weakly similar to MAT8_HUMAN CHLOR | 9.163 |
| 459204 | AW194601 | Hs.13219 | ESTs | 1.000 |
| 439842 | AI910896 | Hs.132413 | ESTs | 1.000 |
| 443113 | AI040686 | Hs.132908 | ESTs | 0.069 |
| 423853 | AB011537 | Hs.133466 | slit (Drosophila) homolog 1 | 0.100 |
| 420792 | AA280321 | Hs.13392 | tethering factor SEC34 | 16.103 |
| 427719 | AI393122 | Hs.134726 | ESTs | 0.667 |
| 443861 | AW449462 | Hs.134743 | ESTs | 5.100 |
| 447578 | AA912347 | Hs.136585 | ESTs, Weakly similar to JC5314 CDC28/cdc | 1.691 |
| 445550 | AI242754 | Hs.137306 | ESTs | 0.006 |
| 454284 | AW297935 | Hs.138493 | ESTs, Moderately similar to ALU7_HUMAN A | 0.003 |
| 418937 | T71508 | Hs.13861 | ESTs, Weakly similar to T42383 probable | 0.042 |
| 424098 | AF077374 | Hs.139322 | small proline-rich protein 3 | 1.347 |
| 453370 | AI470523 | Hs.139336 | ATP-binding cassette, sub-family C (CFTR | 0.186 |
| 424099 | AF071202 | Hs.139336 | ATP-binding cassette, sub-family C (CFTR | 47.949 |
| 426900 | AW163564 | Hs.142375 | ESTs | 0.404 |
| 439337 | AA448718 | Hs.142505 | ESTs | 0.012 |
| 427961 | AW293165 | Hs.143134 | ESTs | 0.073 |
| 419888 | AI243493 | Hs.144049 | ESTs | 11.958 |
| 413943 | AW294416 | Hs.144687 | Homo sapiens cDNA FLJ12981 fis, clone NT | 77.269 |
| 445871 | AI702901 | Hs.145582 | ESTs, Weakly similar to FOR4 MOUSE FORMI | 183.782 |
| 445911 | AI985987 | Hs.145645 | ESTs, Moderately similar to ALU1_HUMAN A | 0.362 |
| 424395 | M165082 | Hs.146388 | microtubule-associated protein 7 | 203.038 |
| 424411 | NM_005209 | Hs.146549 | crystallin, beta A2 | 1.808 |
| 444517 | AI939339 | Hs.146883 | ESTs | 0.004 |
| 445020 | AI205655 | Hs.147221 | ESTs | 0.307 |
| 422109 | S73265 | Hs.1473 | gastrin-releasing peptide | 1.000 |
| 445352 | AI221087 | Hs.147761 | ESTs | 0.015 |
| 444444 | AI149332 | Hs.14855 | ESTs | 140.859 |
| 444152 | AI125694 | Hs.149305 | hypothetical protein MGC2603 | 2.037 |
| 446248 | AI283014 | Hs.149638 | ESTs | 0.018 |
| 433159 | A9035898 | Hs.150587 | kinesin-like protein 2 | 0.110 |
| 428004 | AA449563 | Hs.151393 | glutamate-cysteine ligase, catalytic sub | 3.179 |
| 456840 | H03754 | Hs.152213 | wingless-type MMTV integration site fami | 0.005 |
| 456844 | AI264155 | Hs.152981 | CDP-diacylglycerol synthase (phosphatida | 1.111 |
| 425206 | NM_002153 | Hs.155109 | hydroxysteroid (17-beta) dehydrogenase 2 | 257.949 |
| 446082 | AI274139 | Hs.156452 | ESTs | 0.779 |
| 444946 | AW139205 | Hs.156457 | hypothetical protein FLJ22408 | 1.919 |
| 446636 | AC002563 | Hs.15767 | citron (rho-interacting, serine/threonin | 0.417 |
| 447073 | AW204821 | Hs.157726 | ESTs | 10.349 |
| 422765 | AW409701 | Hs.1578 | baculoviral IAP repeat-containing 5 (sur | 1.839 |
| 446673 | NM_016361 | Hs.15871 | LPAP for lysophosphatidic acid phosphata | 1.691 |
| 447475 | AI380797 | Hs.158992 | ESTs | 44.641 |
| 425776 | U25128 | Hs.159499 | parathyroid hormone receptor 2 | 0.340 |
| 418343 | AA216372 | Hs.159501 | ESTs | 0.023 |
| 441143 | AI027604 | Hs.159650 | ESTs | 0.280 |
| 440917 | AA909651 | Hs.160025 | ESTs | 1.000 |
| 418365 | AW014345 | Hs.161690 | ESTs | 0.066 |
| 431839 | AW020280 | Hs.162025 | ESTs | 0.005 |
| 446839 | BE091926 | Hs.16244 | mitotic spindle coiled-coil related prot | 0.606 |
| 438817 | AI023799 | Hs.163242 | ESTs | 2.202 |
| 432441 | AW292425 | Hs.163484 | ESTs | 2.305 |
| 442577 | AA292998 | Hs.163900 | ESTs | 688.038 |
| 435212 | AW300100 | Hs.164185 | ESTs | 0.002 |
| 425048 | H05468 | Hs.164502 | ESTs | 0.083 |
| 442083 | R50192 | Hs.165062 | ESTs | 3.844 |
| 423536 | L22075 | Hs.1666 | guanine nucleotide binding protein (G pr | 0.157 |
| 418678 | NM_001327 | Hs.167379 | cancer/testis antigen | 269.487 |
| 446989 | AK001898 | Hs.16740 | hypothetical protein FLJ11036 | 0.208 |
| 456967 | AW004056 | Hs.168357 | T-box 2 | 160.397 |
| 447979 | AI457197 | Hs.170348 | ESTs | 0.016 |
| 458814 | AI498957 | Hs.170861 | ESTs, Weakly similar to Z195_HUMAN ZINC | 1.036 |
| 446312 | BE087853 | Hs.171802 | ESTs, Weakly similar to T08729 RING zinc | 1.334 |
| 426783 | Z19084 | Hs.172210 | MUF1 protein | 1.654 |
| 423916 | AW993496 | Hs.17235 | Homo sapiens clone TCCCIA00176 mRNA sequ | 154.064 |
| 409092 | AI735283 | Hs.172608 | ESTs | 0.007 |
| 426853 | U32974 | Hs.172777 | baculoviral IAP repeat-containing 4 | 0.009 |
| 426968 | U07616 | Hs.173034 | amphiphysin (Stiff-Mann syndrome with br | 0.002 |
| 407581 | R48402 | Hs.173508 | P3ECSL | 0.866 |
| 427239 | BE270447 | Hs.174070 | ubiquitin carrier protein | 15.708 |
| 427268 | X78520 | Hs.174139 | chloride channel 3 | 207.936 |
| 436577 | W84774 | Hs.17643 | ESTs | 62.333 |
| 420876 | AA918425 | Hs.177744 | ESTs | 32.959 |
| 427528 | AU077143 | Hs.179565 | minichromosome maintenance deficient (S. | 1.171 |
| 427585 | D31152 | Hs.179729 | collagen, type X, alpha 1 (Schmid metaph | 1.000 |
| 427747 | AW411425 | Hs.180655 | serine/threonine kinase 12 | 12.446 |
| 429813 | AW139678 | Hs.180791 | ESTs | 0.013 |
| 439806 | AA846824 | Hs.180908 | ESTs | 0.561 |
| 427878 | C05766 | Hs.181022 | CGI-07 protein | 0.002 |
| 440284 | AA912032 | Hs.181059 | ESTs, Weakly similar to 2108276A ssDNA-b | 0.030 |
| 427922 | AK001934 | Hs.181112 | HSPC126 protein | 0.039 |
| 427972 | AA864870 | Hs.181304 | putative gene product | 0.004 |
| 428071 | AF212848 | Hs.182339 | ets homologous factor | 4.321 |
| 428336 | AA503115 | Hs.183752 | microseminoprotein, beta- | 145.128 |
| 428450 | NM_014791 | Hs.184339 | KIAA0175 gene product | 0.370 |
| 428479 | Y00272 | Hs.184572 | cell division cycle 2, G1 to S and G2 to | 0.632 |
| 438746 | AI885815 | Hs.184727 | ESTs | 0.339 |
| 420557 | AA960844 | Hs.186579 | Homo sapiens , clone IMAGE: 4081483, mRNA | 0.006 |
| 431014 | W67730 | Hs.187573 | ESTs | 0.344 |
| 428651 | AF196478 | Hs.188401 | annexin A10 | 1.459 |
| 416225 | AA577730 | Hs.188684 | ESTs, Weakly similar to PC4259 ferritin | 0.502 |
| 432497 | AA551104 | Hs.189048 | ESTs, Moderately similar to ALUC_HUMAN ! | 2.499 |
| 431474 | AL133990 | Hs.190642 | ESTs | 0.044 |
| 427742 | AA411880 | Hs.190888 | ESTs | 0.158 |
| 428058 | AI821625 | Hs.191602 | ESTs | 0.006 |
| 431245 | AA496933 | Hs.191687 | ESTs | 0.006 |
| 453204 | R10799 | Hs.191990 | ESTs | 1.734 |
| 436608 | AA628980 | Hs.192371 | down syndrome critical region protein DS | 115.500 |
| 447342 | AI199268 | Hs.19322 | Homo sapiens , Similar to RIKEN cDNA 2010 | 0.152 |
| 454032 | W31790 | Hs.194293 | ESTs, Weakly similar to I54374 gene NF2 | 60.103 |
| 449121 | AI915858 | Hs.194980 | ESTs | 0.003 |
| 447827 | U73727 | Hs.19718 | protein tyrosine phosphatase, receptor t | 305.974 |
| 438401 | AL046321 | Hs.197484 | ESTs, Weakly similar to JC4296 ring fing | 0.002 |
| 457200 | U33749 | Hs.197764 | thyroid transcription factor 1 | 0.011 |
| 429211 | AF052693 | Hs.198249 | gap junction protein, beta 5 (connexin 3 | 9.390 |
| 429257 | AW163799 | Hs.198365 | 2,3-bisphosphoglycerate mutase | 178.436 |
| 429276 | AF056085 | Hs.198612 | G protein-coupled receptor 51 | 0.729 |
| 449818 | AW594365 | Hs.199365 | ESTs | 1.000 |
| 429345 | R11141 | Hs.199695 | hypothetical protein | 7.339 |
| 443564 | AI921685 | Hs.199713 | ESTs | 0.001 |
| 449847 | AW204447 | Hs.199750 | organic anion transporter polypeptide-re | 1.000 |
| 449351 | AW016537 | Hs.200760 | ESTs | 0.005 |
| 426322 | J05068 | Hs.2012 | transcobalamin I (vitamin B12 binding pr | 381.474 |
| 434411 | AA632649 | Hs.201372 | ESTs | 0.039 |
| 448045 | AJ297436 | Hs.20166 | prostate stem cell antigen | 2.337 |
| 446555 | AV659046 | Hs.201847 | ESTs | 0.024 |
| 450411 | D61167 | Hs.202156 | ESTs | 0.004 |
| 442282 | AW451086 | Hs.202390 | ESTs | 1.000 |
| 427587 | BE348244 | Hs.202628 | ESTs, Weakly similar to I78885 serine/th | 228.705 |
| 429486 | AF155827 | Hs.203963 | hypothetical protein FLJ10339 | 0.133 |
| 426682 | AV660038 | Hs.2056 | UDP glycosyltransferase 1 family, polype | 2.070 |
| 426746 | J03626 | Hs.2057 | uridine monophosphate synthetase (orotat | 0.528 |
| 448275 | BE514434 | Hs.20830 | kinesin-like 2 | 19.718 |
| 459058 | H85939 | Hs.209605 | EST | 0.005 |
| 441795 | N58115 | Hs.21137 | AD024 protein | 1.000 |
| 451592 | AI805416 | Hs.213897 | ESTs | 0.012 |
| 443367 | AW071349 | Hs.215937 | ESTs | 0.003 |
| 415949 | H10562 | Hs.21691 | ESTs | 0.072 |
| 444008 | BE544855 | Hs.220756 | ESTs, Weakly similar to SFR4_HUMAN SPLIC | 213.962 |
| 432548 | AW973399 | Hs.22133 | hypothetical protein FLJ20121 | 0.250 |
| 427867 | NM_005073 | Hs.2217 | solute carrier family 15 (oligopeptide t | 0.010 |
| 453123 | AI953718 | Hs.221849 | ESTs | 0.566 |
| 439569 | AW602166 | Hs.222399 | CEGP1 protein | 10.625 |
| 428227 | AA321649 | Hs.2248 | small inducible cytokine subfamily B (Cy | 27.603 |
| 435956 | AF269255 | Hs.22604 | lysosomal apyrase-like protein 1 | 127.564 |
| 453883 | AI638516 | Hs.22630 | cofactor required for Sp 1 transcriptiona | 2.216 |
| 431253 | R06428 | Hs.226351 | ESTs | 0.023 |
| 453900 | AW003582 | Hs.226414 | ESTs, Weakly similar to ALU8_HUMAN ALU S | 0.103 |
| 430034 | X60155 | Hs.227767 | zinc finger protein 41 | 1.000 |
| 423017 | AW178761 | Hs.227948 | sense (or cysteine) proteinase inhibito | 0.024 |
| 417997 | AA418189 | Hs.23017 | Homo sapiens cDNA: FLJ22747 fis, clone K | 1.635 |
| 441362 | BE614410 | Hs.23044 | RAD51 (S. cerevisiae) homolog (E coli Re | 120.167 |
| 452956 | AW003578 | Hs.231872 | ESTs | 1.000 |
| 446009 | AI989885 | Hs.231926 | ESTs | 4.000 |
| 430499 | AW969408 | Hs.231991 | ESTs | 0.014 |
| 448560 | BE613183 | Hs.23213 | ESTs | 285.090 |
| 441508 | AW015203 | Hs.232237 | ESTs | 0.261 |
| 453228 | AW628325 | Hs.232327 | ESTs | 1.000 |
| 442167 | H18740 | Hs.23248 | hypothetical protein from EUROIMAGE 2005 | 0.240 |
| 453321 | AI984381 | Hs.232521 | ESTs | 0.609 |
| 449207 | AL044222 | Hs.23255 | nucleoporin 155 kD | 0.551 |
| 430152 | AB001325 | Hs.234642 | aquaponin 3 | 1.040 |
| 439239 | AI031540 | Hs.235331 | ESTs | 0.598 |
| 435087 | AW975241 | Hs.23567 | ESTs | 0.007 |
| 451276 | AW294386 | Hs.236533 | ESTs, Highly similar to dJ742C19.2 [H. sa | 0.012 |
| 447343 | AA256641 | Hs.236894 | ESTs, Highly similar to S02392 alpha-2-m | 1.280 |
| 431011 | AA490631 | Hs.23783 | ESTs | 0.016 |
| 430307 | BE513442 | Hs.238944 | hypothetical protein FLJ10631 | 284.526 |
| 444371 | BE540274 | Hs.239 | forkhead box M1 | 3.691 |
| 424264 | D80400 | Hs.239388 | Human DNA sequence from clone RP1-B14 | 0.255 |
| 449722 | BE280074 | Hs.23960 | cyclin B1 | 0.467 |
| 430486 | BE062109 | Hs.241551 | chloride channel, calcium activated, fam | 13.419 |
| 430168 | AW968343 | Hs.24255 | DKFZP434l1735 protein | 1.192 |
| 452292 | AW139588 | Hs.244369 | ESTs | 1.000 |
| 412661 | N32860 | Hs.24611 | ESTs, Weakly similar to I54374 gene NF2 | 2.500 |
| 456682 | AW500321 | Hs.246766 | Homo sapiens cDNA FLJ12360 fis, clone MA | 0.014 |
| 457343 | NM_013936 | Hs.247862 | olfactory receptor, family 12, subfamily | 0.233 |
| 430978 | U53583 | Hs.248182 | olfactory receptor, family 1, subfamily | 1.000 |
| 431020 | AF097874 | Hs.248226 | caspase 14, apoptosis-related cysteine p | 5.866 |
| 431070 | AW408164 | Hs.249184 | transcription factor 19 (SC1) | 1.838 |
| 431098 | AW501465 | Hs.249230 | ribonuclease L (2′,5′-oligoisoadenylate | 0.004 |
| 454170 | AW177225 | Hs.250158 | ESTs | 0.243 |
| 439223 | AW238299 | Hs.250618 | UL 16 binding protein 2 | 0.516 |
| 438081 | H49546 | Hs.251391 | claudin 16 | 0.080 |
| 431347 | AI133461 | Hs.251664 | insulin-like growth factor 2 (somatomedi | 843.974 |
| 450663 | H43540 | Hs.25292 | ribonuclease HI, large subunit | 5.928 |
| 450684 | AA872605 | Hs.25333 | interleukin 1 receptor, type II | 1.000 |
| 413094 | H24184 | Hs.25413 | TOLLIP protein | 268.885 |
| 450796 | NM_001988 | Hs.25482 | ennoplakin | 1.643 |
| 408827 | AW275730 | Hs.254825 | ESTs | 0.008 |
| 444129 | AW294292 | Hs.256212 | ESTs | 0.002 |
| 430637 | BE160081 | Hs.256290 | S100 calcium-binding protein A11 (calgiz | 3.240 |
| 436138 | H53323 | Hs.25717 | Homo sapiens cDNA: FLJ23454 fis, clone H | 0.679 |
| 450983 | AA305384 | Hs.25740 | ERO1 (S. cerevisiae)-like | 260.231 |
| 453459 | BE047032 | Hs.257789 | ESTs | 2.133 |
| 456536 | AW135986 | Hs.257859 | ESTs | 98.795 |
| 438424 | AI912498 | Hs.25895 | hypothetical protein FLJ14996 | 1.882 |
| 451161 | AA211329 | Hs.26006 | hypothetical protein FLJ10559 | 0.012 |
| 430634 | AI860651 | Hs.26685 | calcyphosine | 9.561 |
| 435562 | AL046988 | Hs.268677 | ESTs, Moderately similar to ALU7_HUMAN A | 0.957 |
| 417964 | R71449 | Hs.268760 | ESTs | 0.004 |
| 445703 | AV654845 | Hs.27 | glycine dehydrogenase (decarboxylating; | 1.324 |
| 431846 | BE019924 | Hs.271580 | uroplakin 1B | 303.679 |
| 453074 | AA031813 | Hs.271880 | ESTs | 0.004 |
| 431890 | X17033 | Hs.271986 | integrin, alpha 2 (CD49B,alpha 2 subuni | 1.828 |
| 435182 | AA669386 | Hs.272035 | ESTs, Weakly similar to gonadotropin ind | 0.013 |
| 430791 | AA486293 | Hs.272068 | ESTs, Weakly similar to ALU3_HUMAN ALU S | 8.978 |
| 432136 | AA157632 | Hs.272630 | vacuolar proton pump delta polypeptide | 0.316 |
| 451939 | U80456 | Hs.27311 | single-minded (Drosophila) homolog 2 | 0.014 |
| 451982 | F13036 | Hs.27373 | Homo sapiens mRNA; cDNA DKFZp564O1763 (f | 26.348 |
| 423031 | AI278995 | Hs.27457 | ESTs | 53.288 |
| 455612 | BE042896 | Hs.274848 | ESTs | 21.013 |
| 452046 | AB018345 | Hs.27657 | KIAA0802 protein | 129.013 |
| 436567 | AI492860 | Hs.276904 | ESTs | 0.007 |
| 459006 | AW298631 | Hs.27721 | Wolf-Hirschhorn syndrome candidate 1-lik | 0.031 |
| 430157 | BE348706 | Hs.278543 | ESTs | 99.244 |
| 452012 | AA307703 | Hs.279766 | kinesin family member 4A | 0.408 |
| 433001 | AF217513 | Hs.279905 | clone HQ0310 PRO0310p1 | 1.721 |
| 458663 | AV658444 | Hs.280776 | tankyrase, TRF1-interacting ankyrin-rela | 38.231 |
| 450020 | AI680684 | Hs.282219 | ESTs | 0.003 |
| 435858 | AF254260 | Hs.283009 | tuftelin 1 | 1.516 |
| 430733 | AW975920 | Hs.283361 | ESTs | 1.000 |
| 446024 | AB040946 | Hs.284227 | KIAA1513 protein | 9.424 |
| 433967 | AF113018 | Hs.284302 | PRO1621 protein | 0.008 |
| 438915 | AA280174 | Hs.285681 | Williams-Beuren syndrome chromosome regi | 0.030 |
| 431958 | X63629 | Hs.2877 | cadherin 3, type 1, P-cadherin (placenta | 1.058 |
| 414595 | AA641726 | Hs.289015 | hypothetical protein MGC4171 | 273.013 |
| 432097 | X51730 | Hs.2905 | progesterone receptor | 0.002 |
| 452345 | AA293279 | Hs.29173 | hypothetical protein FLJ20515 | 4.010 |
| 457733 | AW974812 | Hs.291971 | ESTs | 1.000 |
| 441398 | AA932398 | Hs.292036 | ESTs, Weakly similar to B34087 hypotheti | 1.000 |
| 428182 | BE386042 | Hs.293317 | ESTs, Weakly similar to GGC1_HUMAN G ANT | 0.006 |
| 452401 | NM_007115 | Hs.29352 | tumor necrosis factor, alpha-induced pro | 0.003 |
| 433365 | AF026944 | Hs.293797 | ESTs | 0.049 |
| 417151 | AA194055 | Hs.293858 | ESTs | 6.593 |
| 424242 | AA337476 | Hs.293984 | hypothetical protein MGC13102 | 1.656 |
| 432375 | BE536069 | Hs.2962 | S100 calcium-binding protein P | 17.094 |
| 422424 | AI186431 | Hs.296638 | prostate differentiation factor | 2.646 |
| 432410 | X68561 | Hs.2982 | Sp4 transcription factor | 0.007 |
| 426847 | S78723 | Hs.298623 | 5-hydroxytryptamine (serotonin) receptor | 0.005 |
| 454054 | AI336329 | Hs.301519 | Homo sapiens cDNA FLJ12536 fis, clone NT | 0.488 |
| 452142 | AB028947 | Hs.301654 | KIAA1024 protein | 0.009 |
| 449773 | R76294 | Hs.302383 | ESTs | 0.001 |
| 438366 | AA805760 | Hs.303567 | ESTs | 1.000 |
| 452724 | R84810 | Hs.30464 | cyclin E2 | 1.000 |
| 429343 | AK000785 | Hs.307036 | Homo sapiens , Similar to epsin 3, clone | 0.494 |
| 446466 | H38026 | Hs.308 | arrestin 3, retinal (X-arrestin) | 0.022 |
| 430694 | AA810624 | Hs.30936 | ESTs, Weakly similar to H2BH_HUMAN HISTO | 16.744 |
| 432789 | D26361 | Hs.3104 | KIAA0042 gene product | 0.302 |
| 432666 | AW204069 | Hs.312716 | ESTs, Weakly similar to unnamed protein | 0.001 |
| 453028 | AB006532 | Hs.31442 | RecQ protein-like 4 | 13.392 |
| 433091 | Y12642 | Hs.3185 | lymphocyte antigen 6 complex, locus D | 2.766 |
| 427122 | AW057736 | Hs.323910 | HER2 receptor tyrosine kinase (c-erb-b2, | 553.782 |
| 453216 | AL137566 | Hs.32405 | Homo sapiens mRNA; cDNA DKFZp586G0321 (f | 84.115 |
| 443247 | BE614387 | Hs.333893 | c-Myc target JPO1 | 79.385 |
| 439632 | AW410714 | Hs.334437 | hypothetical protein MGC4248 | 337.474 |
| 431448 | AL137517 | Hs.334473 | hypothetical protein DKFZp564O1278 | 0.842 |
| 411248 | AA551538 | Hs.334605 | Homo sapiens cDNA FLJ14408 fis, clone HE | 402.500 |
| 433958 | AW043909 | Hs.334707 | aminoacylase 1 | 191.179 |
| 432842 | AW674093 | Hs.334822 | hypothetical protein MGC4485 | 313.462 |
| 457292 | AI921270 | Hs.334882 | hypothetical protein FLJ14251 | 21.744 |
| 451359 | H85334 | Hs.336623 | ESTs | 0.038 |
| 440249 | AI246590 | Hs.337275 | ESTs | 0.432 |
| 434487 | AF143867 | Hs.337588 | ESTs, Moderately similar to S65657 alpha | 1.102 |
| 447437 | U07225 | Hs.339 | purinergic receptor P2Y, G-protein coupl | 0.483 |
| 447519 | U46258 | Hs.339665 | ESTs | 1.032 |
| 434192 | AW387314 | Hs.34371 | ESTs | 0.003 |
| 453765 | BE279901 | Hs.35091 | hypothetical protein FLJ10775 | 0.056 |
| 441020 | W79283 | Hs.35962 | ESTs | 75.141 |
| 453884 | AA355925 | Hs.36232 | KIAA0186 gene product | 0.138 |
| 453922 | AF053306 | Hs.36708 | budding uninhibited by benzimidazoles 1 | 0.008 |
| 453945 | NM_005171 | Hs.36908 | activating transcription factor 1 | 0.044 |
| 447289 | AW247017 | Hs.36978 | melanoma antigen, family A, 3 | 0.002 |
| 407626 | U39196 | Hs.37169 | potassium inwardly-rectifying channel, s | 0.009 |
| 423620 | N71320 | Hs.39938 | ESTs | 1.000 |
| 436027 | AI864053 | Hs.39972 | ESTs, Weakly similar to 138588 reverse t | 0.042 |
| 407846 | AA426202 | Hs.40403 | Cbp/p300-interacting transactivator, wit | 1.810 |
| 443133 | AI033878 | Hs.41379 | ESTs | 0.534 |
| 434534 | H90477 | Hs.41407 | ESTs | 0.013 |
| 452934 | AA581322 | Hs.4213 | hypothetical protein MGC16207 | 3.679 |
| 434952 | T10269 | Hs.4285 | Homo sapiens cDNA: FLJ22505 fis, clone H | 2.885 |
| 432237 | AK001926 | Hs.44143 | polybromo 1 | 0.010 |
| 420900 | AL045633 | Hs.44269 | ESTs | 10.436 |
| 408522 | AI541214 | Hs.46320 | Small proline-rich protein SPRK [human, | 3.393 |
| 435099 | AC004770 | Hs.4756 | flap structure-specific endonuclease 1 | 386.256 |
| 431009 | BE149762 | Hs.48956 | gap junction protein, beta 6 (connexin 3 | 0.922 |
| 408947 | AL080093 | Hs.49117 | Homo sapiens mRNA; cDNA DKFZp564N1662 (f | 0.003 |
| 435647 | AI653240 | Hs.49823 | ESTs | 175.910 |
| 435854 | AJ278120 | Hs.4996 | putative ankyrin-repeat containing prote | 2.584 |
| 436291 | BE568452 | Hs.5101 | protein regulator of cytokinesis 1 | 0.610 |
| 455506 | AA703584 | Hs.5105 | hypothetical protein FLJ10569 | 0.008 |
| 436481 | AA379597 | Hs.5199 | HSPC150 protein similar to ubiquitin-con | 1.089 |
| 4092807 | AL080213 | Hs.52792 | Homo sapiens mRNA; cDNA DKFZp586l1823 (f | 16.910 |
| 435047 | AA454985 | Hs.54973 | cadherin-like protein VR20 | 0.612 |
| 439750 | AL359053 | Hs.57664 | Homo sapiens mRNA full length insert cDN | 5.938 |
| 439452 | AA918317 | Hs.57987 | B-cell CLL/lymphoma 11B (zinc finger pro | 1.000 |
| 439482 | W70045 | Hs.58089 | ESTs | 0.118 |
| 439606 | W79123 | Hs.58561 | G protein-coupled receptor 87 | 0.095 |
| 439706 | AW872527 | Hs.59761 | ESTs, Weakly similar to DAP1_HUMAN DEATH | 0.055 |
| 452240 | AI591147 | Hs.61232 | ESTs | 0.221 |
| 452316 | AA298484 | Hs.61265 | ESTs, Moderately similar to G786_HUMAN P | 2.595 |
| 452747 | BE153855 | Hs.61460 | Ig superfamily receptor LNIR | 3.677 |
| 434876 | AF160477 | Hs.61460 | Ig superfamily receptor LNIR | 7.587 |
| 444783 | AK001468 | Hs.62180 | anillin (Drosophila Scraps homolog), act | 0.046 |
| 438779 | NM_003787 | Hs.6414 | nucleolar protein 4 | 0.030 |
| 439453 | BE264974 | Hs.6566 | thyroid hormone receptor interactor 13 | 12.016 |
| 440126 | AA975145 | Hs.66194 | ESTs | 0.008 |
| 451291 | R39288 | Hs.6702 | ESTs | 0.012 |
| 439963 | AW247529 | Hs.6793 | platelet-activating factor acetylhydrola | 1.653 |
| 440006 | AK000517 | Hs.6844 | hypothetical protein FLJ20510 | 8.628 |
| 437044 | AL035864 | Hs.69517 | cDNA for differentially expressed CO16 g | 140.908 |
| 418107 | R41726 | Hs.7284 | ESTs | 0.146 |
| 436326 | BE085236 | Hs.75313 | aldo-keto reductase family 1, member B1 | 0.649 |
| 433675 | AW977653 | Hs.75319 | ribonucleotide reductase M2 polypeptide | 0.237 |
| 414416 | AW409985 | Hs.76084 | hypothetical protein MGC2721 | 2.242 |
| 414430 | AI346201 | Hs.76118 | ubiquitin carboxyl-terminal esterase L1 | 0.202 |
| 414682 | AL021154 | Hs.76884 | inhibitor of DNA binding 3, dominant neg | 2.318 |
| 414807 | AI738616 | Hs.77348 | hydroxyprostaglandin dehydrogenase 15-(N | 1.622 |
| 414907 | X90725 | Hs.77597 | polo (Drosophia)-like kinase | 246.564 |
| 451575 | AA767622 | Hs.78893 | KIAA0244 protein | 1.000 |
| 400303 | AA242758 | Hs.79136 | LIV-1 protein, estrogen regulated | 4.552 |
| 416498 | U33632 | Hs.79351 | potassium channel, subfamily K, member 1 | 5.128 |
| 458921 | AI682088 | Hs.79375 | holocarboxylase synthetase (biotin-[prop | 0.246 |
| 409235 | AA188827 | Hs.7988 | ESTs, Weakly similar to I38022 hypotheti | 7.249 |
| 440371 | BE268550 | Hs.80449 | Homo sapiens , clone IMAGE: 3535294, mRNA, | 0.792 |
| 452732 | BE300078 | Hs.80449 | Homo sapiens , clone IMAGE: 3535294, mRNA, | 360.782 |
| 417003 | AL038170 | Hs.80756 | betaine-homocysteine methyltransferase | 60.590 |
| 407584 | W25945 | Hs.8173 | hypothetical protein FLJ10803 | 9.988 |
| 417312 | AW888411 | Hs.81915 | leukemia-associated phosphoprotein p18 ( | 402.705 |
| 417389 | BE260964 | Hs.82045 | midkine (neurite growth-promoting factor | 10.806 |
| 428839 | AI767756 | Hs.82302 | Homo sapiens cDNA FLJ14814 fis, clone NT | 1.051 |
| 438315 | R56795 | Hs.82419 | ESTs | 0.226 |
| 417900 | BE250127 | Hs.82906 | CDC20 (cell division cycle 20, S. cerevi | 26.260 |
| 417933 | X02308 | Hs.82962 | thymidylate synthetase | 221.090 |
| 418067 | AI127958 | Hs.83393 | cystatin E/M | 2.396 |
| 438086 | AA336519 | Hs.83623 | nuclear receptor subfamily 1, group I, m | 1.000 |
| 418205 | L21715 | Hs.83760 | troponin I, skeletal, fast | 0.159 |
| 413385 | M34455 | Hs.840 | indoleamine-pyrrole 2,3 dioxygenase | 0.490 |
| 418322 | AA284166 | Hs.84113 | cyclin-dependent kinase inhibitor 3 (CDK | 3.527 |
| 413529 | U11874 | Hs.846 | interleukin 8 receptor, beta | 0.077 |
| 458027 | L49054 | Hs.85195 | myeloid leukemia factor 1 | 0.008 |
| 418543 | NM_005329 | Hs.85962 | hyaluronan synthase 3 | 1.813 |
| 418583 | AA604379 | Hs.86211 | hypothetical protein | 125.769 |
| 441801 | AW242799 | Hs.86366 | ESTs | 55.026 |
| 414792 | BE314949 | Hs.87128 | hypothetical protein FLJ23309 | 8.139 |
| 407246 | S70348 | Hs.87149 | integrin, beta 3 (platelet glycoprotein | 0.020 |
| 433417 | AA587773 | Hs.8859 | Homo sapiens , Similar to RIKEN cDNA 5830 | 313.141 |
| 445060 | AA830811 | Hs.88808 | ESTs | 1.000 |
| 453450 | AW797627 | Hs.89474 | ADP-ribosylation factor 6 | 137.718 |
| 419227 | BE537383 | Hs.89739 | cholinergic receptor, nicotinic, beta po | 0.006 |
| 401464 | AF039241 | Hs.9028 | histone deacetylase 5 | 6.846 |
| 443162 | T49951 | Hs.9029 | DKFZP434G032 protein | 14.057 |
| 431024 | AA713666 | Hs.90462 | Homo sapiens , clone IMAGE: 4132043, mRNA, | 3.507 |
| 419559 | Y07828 | Hs.91096 | ring finger protein | 0.025 |
| 419741 | NM_007019 | Hs.93002 | ubiquitin carrier protein E2-C | 684.577 |
| 443426 | AF098158 | Hs.9329 | chromosome 20 open reading frame 1 | 0.363 |
| 424457 | AI249036 | Hs.94292 | hypothetical protein FLJ23311 | 175.667 |
| 410348 | AW182663 | Hs.95469 | ESTs | 0.011 |
| 439738 | BE246502 | Hs.9598 | sema domain, immunoglobulin domain (Ig), | 428.231 |
| 421478 | AI683243 | Hs.97258 | ESTs, Moderately similar to S29539 ribos | 0.005 |
| 443767 | BE562136 | Hs.9736 | proteasome (prosome, macropain) 26S subu | 1.168 |
| 426902 | AI125334 | Hs.97408 | ESTs | 37.467 |
| 444874 | AI218496 | Hs.97515 | BRCA1-interacting protein 1; BRCA1-assoc | 0.067 |
| 427356 | AW023482 | Hs.97849 | ESTs | 1.000 |
| 430000 | AW205931 | Hs.99598 | hypothetical protein MG05338 | 0.812 |
| 419485 | AA489023 | Hs.99807 | ESTs, Weakly similar to unnamed protein | 0.342 |
| 420783 | AI659838 | Hs.99923 | lectin, galactoside-binding, soluble, 7 | 19.785 |
| 421934 | AA300625 | | gb: EST13476 Testis tumor Homo sapiens cD | 103.769 |
| 431322 | AW970622 | | gb: EST382704 MAGE resequences, MAGK Homo | 0.258 |
| 424040 | AA334400 | | gb: EST38610 Embryo, 9 week Homo sapiens | 0.011 |
| 433108 | AB002446 | | gb: Homo sapiens mRNA from chromosome 5q2 | 0.023 |
| 458829 | AI557388 | | gb: PT2.1_6_G03.r tumor2 Homo sapiens cDN | 1.000 |
| 459169 | AI905517 | | gb: RC-BT091-210199-105 BT091 Homo sapien | 0.773 |
| 400300 | X03363 | | HER2 receptor tyrosine kinase (c-erb-b2, | 468.462 |
| 440012 | AA861072 | | gb: ak32e05.s1 Soares_testis_NHT Homo sap | 0.002 |
| 412799 | AI267606 | | gb: aq91h03.x1 Stanley Frontal SB pool 1 | 0.010 |
| 412964 | BE019688 | | gb: bb28g08.x1 NIH_MGC_5 Homo sapiens cDN | 0.003 |
| 406992 | S82472 | | gb: beta -pol = DNA polymerase beta (exon a | 0.005 |
| 414969 | 016195 | | gb: C16195 Clontech human aorta polyA mRN | 0.023 |
| 413158 | BE068098 | | gb: CM1-BT0368-061299-060-c09 BT0368 Homo | 0.007 |
| 453823 | AL137967 | | gb: DKFZp761D2315_r1 761 (synonym: hamy2) | 0.994 |
| 453846 | AL157586 | | gb: DKFZp761H0216_r1 761 (synonym: hamy2) | 0.004 |
| 407055 | X89211 | | gb: H. sapiens DNA for endogenous retrovir | 0.037 |
| 415204 | T27434 | | gb: hbc2294 Human pancreatic islet Homo s | 76.500 |
| 434572 | AF147340 | | gb: Homo sapiens full length insert cDNA | 0.030 |
| 438990 | AF085890 | | gb: Homo sapiens full length insert cDNA | 1.000 |
| 439780 | AL109688 | | gb: Homo sapiens mRNA full length insert | 0.256 |
| 413671 | Z43712 | | gb: HSC1JA121 normalized infant brain cDN | 0.009 |
| 406974 | M57293 | | gb: Human parathyroid hormone-related pep | 0.004 |
| 455797 | BE091833 | | gb: IL2-BT0731-260400-076-F04 BT0731 Homo | 2.616 |
| 455807 | BE141140 | | gb: MR0-HT0075-021299-006-d07 HT0075 Homo | 0.413 |
| 432189 | AA527941 | | gb: nh30c04.s1 NCI_CGAP_Pr3 Homo sapiens | 0.015 |
| 443309 | AI821874 | | gb: nt58f10.x5 NCI_CGAP_Pr3 Homo sapiens | 0.007 |
| 437240 | AA747537 | | gb: nx85c05.s1 NCI_CGAP_GCB1 Homo sapiens | 0.006 |
| 455189 | AW864176 | | gb: PM0-SN0014-260400-002-b08 SN0014 Homo | 0.069 |
| 444163 | AI126098 | | gb: qc54g07.x1 Soares_placenta_8to9weeks_{—} | 394.282 |
| 455170 | AW860972 | | gb: QV0-CT0387-180300-167-h07 CT0387 Homo | 0.757 |
| 454789 | BE156314 | | gb: QVO-HT0367-150200-114-d02 HT0367 Homo | 1.000 |
| 433005 | AW936074 | | gb: QV1-DT0069-010200-057-c12 DT0069 Homo | 0.013 |
| 455380 | BE160188 | | gb: QV1.HT0413-010200-059-g05 HT0413 Homo | 0.249 |
| 455650 | BE064655 | | gb: RC1-BT0313-301299-012-c09 BT0313 Homo | 1.000 |
| 436383 | BE065178 | | gb: RC1-BT0314-020200-012-h01 BT0314 Homo | 1.000 |
| 413100 | BE065208 | | gb: RC1-BT0314-310300-015-b09 BT0314 Homo | 271.372 |
| 428436 | BE080180 | | gb: RC4-BT0629-120200-011-b10 BT0629 Homo | 0.002 |
| 455831 | BE144966 | | gb: RC6-HT0187-201099-031-c04 HT0187 Homo | 0.011 |
| 434414 | AI798376 | | gb: tr34b07.x1 NCI_CGAP_Ov23 Homo sapiens | 293.654 |
| 414221 | AW450979 | | gb: UI-H-BI3-ala-a-12-0-UI.s1 NCI_CGAP_Su | 0.807 |
| 409488 | AW402825 | | gb: UI-HF-BK0-aaq-d-08-0-UI.r1 NIH_MGC_36 | 0.965 |
| 437938 | AI950087 | | gb: wq05c02.x1 NCI_CGAP_Kid12 Homo sapien | 2.952 |
| 451385 | AA017656 | | gb: ze39h01.r1 Soares retina N2b4HR Homo | 7.341 |
| 449325 | AA001162 | | gb: ze48b06.r1 Soares retina N2b4HR Homo | 0.004 |
| 413316 | W91931 | | gb: zh47c01.r1 Soares_fetal_liver_spleen_{—} | 0.004 |
| 401016 | | | | 0.342 |
| 401335 | | | | 0.256 |
| 401555 | | | | 1.000 |
| 401760 | | | | 301.372 |
| 401781 | | | | 247.141 |
| 401961 | | | | 1.722 |
| 402239 | | | | 5.180 |
| 402305 | | | | 0.917 |
| 402424 | | | | 551.141 |
| 402777 | | | | 153.231 |
| 402778 | | | | 0.006 |
| 402837 | | | | 0.367 |
| 402948 | | | | 154.103 |
| 402952 | | | | 17.038 |
| 403142 | | | | 0.196 |
| 403297 | | | | 12.744 |
| 403637 | | | | 0.304 |
| 403657 | | | | 0.032 |
| 404136 | | | | 0.008 |
| 404249 | | | | 0.065 |
| 404875 | | | | 1.105 |
| 404917 | | | | 69.590 |
| 404983 | | | | 1.000 |
| 405238 | | | | 1.000 |
| 405364 | | | | 294.141 |
| 405531 | | | | 1.747 |
| 405601 | | | | 145.551 |
| 405621 | | | | 0.224 |
| 405932 | | | | 1.968 |
| 406117 | | | | 0.333 |
| 406354 | | | | 1.000 |
| 406548 | | | | 0.002 |
| 406599 | | | | 0.010 |
| 459702 | AI204995 | | | 0.449 |

Pkey: Unique Eos probeset identifier number
ExAccn: Exemplar Accession number, Genbank accession number
UnigeneID: Unigene number
Unigene Title: Unigene gene title
R1: Ratio of mRNA expression in bladder tumors compared to normal bladder

TABLE 2B

| Pkey | CAT Number | Accession |
|---|---|---|
| 409488 | 1134791_1 | AW402825 BE544338 |
| 412799 | 132817_1 | AI267606 AA121045 AA126521 |
| 412964 | 1339278_1 | BE019688 BE144460 |
| 413100 | 1349119_1 | BE065208 BE065224 BE065168 BE065313 |
| 413158 | 1351251_1 | BE068098 BE068119 BE068083 BE068088 BE068120 BE068155 BE068111 |
| 413316 | 1360169_1 | W91931 W94979 BE081744 |
| 413671 | 1382504_1 | Z43712 BE156729 BE156538 BE156731 BE156673 BE156539 BE156674 BE156430 BE156672 BE156675 BE156432 |
| | | BE156541 |
| 414221 | 142696_1 | AW450979 AA136653 AA136656 AW419381 AA984358 AA492073 BE168945 AA809054 AW238038 |
| | | BE011212 BE011359 BE011367 BE011368 BE011362 BE011215 BE011365 BE011363 |
| 414969 | 1510393_1 | C16195 C16230 C16211 C16164 C16251 |
| 415204 | 1529407_1 | T27434 Z25288 F00323 D82802 R85077 |
| 421934 | 209339_1 | AA300625 R16859 R16860 AW898335 W24337 |
| 424040 | 234659_1 | AA334400 AA334257 AW966124 |
| 428436 | 291472_1 | BE080180 AW827313 AW231970 AA995028 AA428584 AW872716 AW892508 AW854593 AA578441 |
| | | AW975234 AA664937 AA984131 AA528743 AA552874 AA564758 AW063245 AI267534 AW070190 AW893483 |
| | | AA770330 AA906928 AA906582 AA758746 AA551717 AW063311 AA429538 |
| 431322 | 331543_1 | AW970622 AA503009 AA502998 AA502989 AA502805 T92188 |
| 432189 | 342819_1 | AA527941 AI810608 AI620190 AA635266 |
| 433005 | 357346_1 | AW939074 AW939073 BE160476 AW939938 AW939206 AW940012 AW939076 AA573577 AW750479 AA574383 |
| | | AW970057 |
| 433108 | 35896_1 | AB002446 T03146 |
| 434414 | 38585_1 | AI798376 S46400 AW811617 AW811616 W00557 BE142245 AW858232 AW861851 AW858362 AA232351 |
| | | AA218567 AA055556 AW858231 AW857541 AW814172 H66214 AW814398 AF134164 AA243093 |
| | | AA173345 AA199942 AA223384 AA227092 AA227080 T12379 AA092174 T61139 AA149776 AA699829 AW879188 |
| | | AW813567 AW813538 AI267168 AA57718 AA157719 AA100472 AA100774 AA130756 AA157705 AA157730 AA157715 |
| | | AA053524 AW849581 AW854566 C05254 AW882836 T92637 AW812621 AA206583 AA209204 BE156909 |
| | | AA226824 AI829309 AW991957 N66951 AA527374 H66215 AA045564 AI694265 H60808 AA149726 AW195620 |
| | | BE081333 BE073424 AW817662 AW817705 AW817703 AW817659 BE081531 H59570 |
| 434572 | 38911_1 | AF147340 T51948 T52029 |
| 436383 | 41888_1 | BE065178 AJ227879 |
| 437240 | 435139_1 | AA747537 BE089068 BE089070 |
| 437938 | 44573_2 | AI950087 N70208 R97040 N36809 AI308119 AW967677 N35320 AI251473 H59397 AW971573 R97278 |
| | | W01059 AW967671 AA908598 AA251875 AI820501 AI820532 W87891 T85904 U71456 T82391 BE328571 |
| | | T75102 R34725 AA884922 BE328517 AI219788 AA884444 N92578 F13493 AA927794 AI560251 AW874068 |
| | | AL134043 AW235363 AA663345 AW008282 AA488964 AA283144 AI890387 AI950344 AI741346 AI689062 |
| | | AA282915 AW102898 AI872193 AI763273 AW173586 AW150329 AI653832 AI762688 AA988777 AA488892 |
| | | AI356394 AW103813 AI539642 AA642789 AA856975 AW505512 AI961530 AW629970 BE612881 AW276997 |
| | | AW513601 AW512843 AA044209 AW856538 AA180009 AA337499 AW961101 AA251669 AA251874 AI819225 |
| | | AW205862 AI683338 AI858509 AW276905 AI633006 AA972584 AA908741 AW072629 AW513996 AA293273 |
| | | AA969759 N75628 N22388 H84729 H60052 T92487 AI022058 AA780419 AA551005 W80701 AW613456 AI373032 |
| | | AI564269 F00531 H83488 W37181 W78802 R66056 AI002839 R67840 AA300207 AW959581 T63226 F04005 |
| 438990 | 46760_1 | AF085890 H29949 H29856 |
| 439780 | 47673_1 | AL109688 R23665 R26578 |
| 440012 | 483290_1 | AA861072 H02819 R25946 |
| 443309 | 56604_1 | AI821874 AI821868 AA630932 AA653897 AA650103 AI821131 AI821124 |
| 444163 | 593658_1 | AI126098 AI184746 AI148521 |
| 449325 | 80480_1 | AA001162 AA018950 AA017505 R84446 |
| 451385 | 86787_1 | AA017656 AA017374 AA019761 |
| 453823 | 982526_1 | AL137967 BE064160 BE064186 |
| 453846 | 983043_1 | AL157586 AL157590 |
| 454789 | 1234742_1 | BE156314 BE156316 AW820750 |
| 455170 | 1256906_1 | AW860972 AW862598 AW862599 AW860988 AW860983 AW860898 AW860925 AW860922 AW860986 AW860984 |
| | | AW860989 |
| 455189 | 1259271_1 | AW864176 AW864133 AW864185 AW864137 |
| 455380 | 1287679_1 | BE160188 AW935785 BE160401 BE160319 BE160313 BE160395 |
| 455650 | 1348720_1 | BE064655 BE153953 |
| 455797 | 1366826_1 | BE091833 BE091874 BE091871 |
| 455807 | 1370914_1 | BE141140 BE141139 BE141105 BE141143 BE141127 BE141202 BE141108 |
| 455831 | 1373969_1 | BE144966 BE144957 BE144958 |
| 458829 | 773443_1 | AI557388 BE158936 |
| 459169 | 920641_1 | AI905517 AI905455 AI905452 |

Pkey: Unique Eos probeset identifier number
CAT number: Gene cluster number
Accession: Genbank accession numbers

TABLE 2C

| Pkey | Ref | Strand | Nt_position |
|---|---|---|---|
| 401016 | 8117441 | Plus | 126234-126359, 128050-128236 |
| 401335 | 9884881 | Plus | 15736-16352 |
| 401555 | 8099284 | Minus | 162520-162657 |
| 401760 | 9929699 | Plus | 83126-83250, 85320-85540, 94719-95287 |
| 401781 | 7249190 | Minus | 83215-83435, 83531-83656, 83740-83901, 84237-84393, 84955-85037, 86290-86814 |
| 401961 | 4581193 | Minus | 124054-124209 |
| 402239 | 7690131 | Plus | 38175-38304, 42133-42266 |
| 402305 | 7328724 | Plus | 40832-41362 |
| 402424 | 9796344 | Minus | 64925-65073 |
| 402777 | 9588235 | Plus | 126786-126948 |
| 402778 | 9588235 | Plus | 128560-128702 |
| 402837 | 9369121 | Minus | 2013-2186, 9570-9758, 11136-11309, 19429-19677, 21210-21455, 23368-23562, |
| | | | 24342-24527, 29132-29320 |
| 402948 | 9368458 | Minus | 143456-143626, 143808-143935 |
| 402952 | 9408724 | Minus | 119452-119619 |
| 403142 | 9444521 | Plus | 89286-90131 |
| 403297 | 8096824 | Minus | 16584-17264 |
| 403637 | 8671936 | Minus | 142647-142771, 145531-145762 |
| 403657 | 8843996 | Minus | 156223-156370 |
| 404136 | 6981900 | Minus | 42538-46428 |
| 404249 | 8655533 | Plus | 64270-64633 |
| 404875 | 9801324 | Plus | 96588-96732, 97722-97831 |
| 404917 | 7341851 | Plus | 49330-49498 |
| 404983 | 4432779 | Minus | 51178-51374, 52000-52173 |
| 405238 | 7249119 | Minus | 51728-51836 |
| 405364 | 2281075 | Minus | 48325-48491, 49136-49252 |
| 405531 | 9665194 | Plus | 35602-35803 |
| 405601 | 5815493 | Minus | 147835-147935, 149220-149299 |
| 405621 | 5523811 | Plus | 59362-59607 |
| 405932 | 7767812 | Minus | 123525-123713 |
| 406117 | 9142932 | Plus | 54304-54584 |
| 406354 | 9256049 | Minus | 2095-2377 |
| 406548 | 7711514 | Minus | 25138-26762 |
| 406599 | 8248616 | Plus | 10933-11086 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 3A
Preferred therapeutic targets for bladder cancer

| Pkey | ExAccn | UnigeneID | Unigene Title | R1 | R2 |
|---|---|---|---|---|---|
| 421948 | L42583 | Hs.334309 | keratin 6A | 14.20 | 1.20 |
| 439926 | AW014875 | Hs.137007 | ESTs | 11.31 | 21.34 |
| 413324 | V00571 | Hs.75294 | corticotropin releasing hormone | 9.15 | 45.75 |
| 421110 | AJ250717 | Hs.1355 | cathepsin E | 9.07 | 45.35 |
| 417308 | H60720 | Hs.81892 | KIAA0101 gene product | 8.50 | 1.99 |
| 431211 | M86849 | Hs.323733 | gap junction protein, beta 2, 26 kD (conn | 8.39 | 1.28 |
| 418406 | X73501 | Hs.84905 | cytokeratin 20 | 8.10 | 40.50 |
| 446619 | AU076643 | Hs.313 | secreted phosphoprotein 1 (osteopontin, | 7.98 | 1.38 |
| 433001 | AF217513 | Hs.279905 | clone HQ0310 PRO0310p1 | 7.67 | 2.12 |
| 408243 | Y00787 | Hs.624 | interleukin 8 | 7.56 | 4.85 |
| 417715 | AW969587 | Hs.86366 | ESTs | 7.45 | 4.70 |
| 417720 | AA205625 | Hs.208067 | ESTs | 7.34 | 9.18 |
| 423673 | BE003054 | Hs.1695 | matrix metalloproteinase 12 (macrophage | 7.30 | 26.07 |
| 418007 | M13509 | Hs.83169 | matrix metalloproteinase 1 (interstitial | 7.12 | 35.60 |
| 413753 | U17760 | Hs.75517 | laminin, beta 3 (nicein (125 kD), kalinin | 6.95 | 4.96 |
| 441633 | AW958544 | Hs.112242 | normal mucosa of esophagus specific 1 | 6.42 | 0.89 |
| 422168 | AA586894 | Hs.112408 | S100 calcium-binding protein A7 (psorias | 6.08 | 3.49 |
| 407242 | M18728 | | gb: Human nonspecific crossreacting antig | 5.96 | 0.96 |
| 405033 | | | C1002652*: gi\|544327\|sp\|Q04799\|FMO5_RABIT | 5.84 | 16.22 |
| 449230 | BE613348 | Hs.211579 | melanoma cell adhesion molecule | 5.82 | 2.28 |
| 406685 | M18728 | | gb: Human nonspecific crossreacting antig | 5.80 | 0.89 |
| 420159 | AI572490 | Hs.99785 | Homo sapiens cDNA: FLJ21245 fis, clone C | 5.77 | 28.85 |
| 415511 | AI732617 | Hs.182362 | ESTs | 5.65 | 28.25 |
| 426028 | NM_001110 | Hs.172028 | a disintegrin and metalloproteinase doma | 5.60 | 6.51 |
| 424008 | R02740 | Hs.137555 | putative chemokine receptor; GTP-binding | 5.59 | 2.33 |
| 428651 | AF196478 | Hs.188401 | annexin A10 | 5.55 | 27.75 |
| 400843 | | | NM_003105*: Homo sapiens sortilin-related | 5.51 | 4.92 |
| 402230 | | | Target Exon | 5.36 | 21.44 |
| 452747 | BE153855 | Hs.61460 | Ig superfamily receptor LNIR | 5.33 | 2.80 |
| 416065 | BE267931 | Hs.78996 | proliferating cell nuclear antigen | 5.17 | 1.98 |
| 428450 | NM_014791 | Hs.184339 | KIAA0175 gene product | 4.90 | 2.63 |
| 418322 | AA284166 | Hs.84113 | cyclin-dependent kinase inhibitor 3 (CDK | 4.77 | 2.35 |
| 412610 | X90908 | Hs.74126 | fatty acid binding protein 6, ileal (gas | 4.77 | 3.71 |
| 418663 | AK001100 | Hs.41690 | desmocollin 3 | 4.74 | 1.48 |
| 414683 | S78296 | Hs.76888 | hypothetical protein MGC12702 | 4.74 | 2.92 |
| 442432 | BE093589 | Hs.38178 | hypothetical protein FLJ23468 | 4.68 | 1.61 |
| 424834 | AK001432 | Hs.153408 | Homo sapiens cDNA FLJ10570 fis, clone NT | 4.65 | 11.63 |
| 431958 | X63629 | Hs.2877 | cadherin 3, type 1, P-cadherin (placenta | 4.63 | 2.06 |
| 423725 | AJ403108 | Hs.132127 | hypothetical protein LOC57822 | 4.55 | 3.35 |
| 401780 | | | NM_005557*: Homo sapiens keratin 16 (foca | 4.49 | 1.62 |
| 424308 | AW975531 | Hs.154443 | minichromosome maintenance deficient (S. | 4.43 | 2.39 |
| 401093 | | | C12000586*: gi\|6330167\|dbj\|BAA86477.1\| (A | 4.40 | 12.94 |
| 417933 | X02308 | Hs.82962 | thymidylate synthetase | 4.35 | 2.29 |
| 418113 | AI272141 | Hs.83484 | SRY (sex determining region Y)-box 4 | 4.32 | 2.82 |
| 412140 | AA219691 | Hs.73625 | RAB6 interacting, kinesin-like (rabkines | 4.24 | 8.15 |
| 401781 | | | Target Exon | 4.15 | 1.31 |
| 425234 | AW152225 | Hs.165909 | ESTs, Weakly similar to I38022 hypotheti | 4.14 | 7.39 |
| 432842 | AW674093 | Hs.334822 | hypothetical protein MGC4485 | 4.12 | 2.51 |
| 408380 | AF123050 | Hs.44532 | diubiquitin | 4.11 | 3.26 |
| 449722 | BE280074 | Hs.23960 | cyclin B1 | 4.09 | 3.72 |
| 420344 | BE463721 | Hs.97101 | putative G protein-coupled receptor | 4.07 | 2.50 |
| 422809 | AK001379 | Hs.121028 | hypothetical protein FLJ10549 | 4.00 | 7.14 |
| 404977 | | | Insulin-like growth factor 2 (somatomedi | 3.89 | 5.17 |
| 400409 | AF153341 | | Homo sapiens winged helix/forkhead trans | 3.88 | 7.29 |
| 429113 | D28235 | Hs.196384 | prostaglandin-endoperoxide synthase 2 (p | 3.87 | 1.61 |
| 444371 | BE540274 | Hs.239 | forkhead box M1 | 3.87 | 2.75 |
| 443171 | BE281128 | Hs.9030 | TONDU | 3.83 | 9.48 |
| 441362 | BE614410 | Hs.23044 | RAD51 (S. cerevisiae) homolog (E coli Re | 3.82 | 2.98 |
| 439963 | AW247529 | Hs.6793 | platelet-activating factor acetylhydrola | 3.77 | 3.83 |
| 407137 | T97307 | | gb: ye53h05.s1 Soares fetal liver spleen | 3.73 | 4.91 |
| 426088 | AF038007 | Hs.166196 | ATPase, Class I, type 8B, member 1 | 3.68 | 7.08 |
| 443426 | AF098158 | Hs.9329 | chromosome 20 open reading frame 1 | 3.68 | 2.29 |
| 414761 | AU077228 | Hs.77256 | enhancer of zeste (Drosophila) homolog 2 | 3.67 | 3.06 |
| 413063 | AL035737 | Hs.75184 | chitinase 3-like 1 (cartilage glycoprote | 3.67 | 1.18 |
| 421508 | NM_004833 | Hs.105115 | absent in melanoma 2 | 3.67 | 3.65 |
| 443162 | T49951 | Hs.9029 | DKFZP434G032 protein | 3.66 | 3.21 |
| 418478 | U38945 | Hs.1174 | cyclin-dependent kinase inhibitor 2A (me | 3.66 | 3.57 |
| 417771 | AA804698 | Hs.82547 | retinoic acid receptor responder (tazaro | 3.62 | 2.73 |
| 441495 | AW294603 | Hs.127039 | ESTs | 3.60 | 2.71 |
| 422282 | AF019225 | Hs.114309 | apolipoprotein L | 3.57 | 3.92 |
| 417079 | U65590 | Hs.81134 | interleukin 1 receptor antagonist | 3.55 | 0.80 |
| 417275 | X63578 | Hs.295449 | parvalbumin | 3.54 | 4.60 |
| 440006 | AK000517 | Hs.6844 | hypothetical protein FLJ20510 | 3.52 | 2.59 |
| 418203 | X54942 | Hs.83758 | CDC28 protein kinase 2 | 3.50 | 3.61 |
| 400289 | X07820 | Hs.2258 | matrix metalloproteinase 10 (stromelysin | 3.50 | 17.50 |
| 404875 | | | NM_022819*: Homo sapiens phospholipase A2 | 3.46 | 3.24 |
| 420005 | AW271106 | Hs.133294 | ESTs | 3.40 | 2.22 |
| 409757 | NM_001898 | Hs.123114 | cystatin SN | 3.39 | 2.93 |
| 427719 | AI393122 | Hs.134726 | ESTs | 3.31 | 2.51 |
| 406690 | M29540 | Hs.220529 | carcinoembryonic antigen-related cell ad | 3.28 | 0.42 |
| 422283 | AW411307 | Hs.114311 | CDC45 (cell division cycle 45, S. cerevis | 3.28 | 2.62 |
| 406081 | | | Target Exon | 3.25 | 13.54 |
| 426514 | BE616633 | Hs.170195 | bone morphogenetic protein 7 (osteogenic | 3.25 | 2.46 |
| 431009 | BE149762 | Hs.48956 | gap junction protein, beta 6 (connexin 3 | 3.23 | 2.88 |
| 429983 | W92620 | Hs.260855 | ESTs | 3.20 | 2.84 |
| 422158 | L10343 | Hs.112341 | protease inhibitor 3, skin-derived (SKAL | 3.18 | 1.54 |
| 426451 | AI908165 | Hs.169946 | GATA-binding protein 3 (T-cell receptor | 3.17 | 5.44 |
| 411945 | AL033527 | Hs.92137 | v-myc avian myelocytomatosis viral oncog | 3.13 | 15.65 |
| 415752 | BE314524 | Hs.78776 | putative transmembrane protein | 3.11 | 2.46 |
| 408633 | AW963372 | Hs.46677 | PRO2000 protein | 3.11 | 3.30 |
| 409956 | AW103364 | Hs.727 | inhibin, beta A (activin A, activin AB a | 3.09 | 1.52 |
| 412420 | AL035668 | Hs.73853 | bone morphogenetic protein 2 | 3.08 | 2.22 |
| 400297 | AI127076 | Hs.306201 | hypothetical protein DKFZp564O1278 | 3.05 | 12.49 |
| 437931 | AI249468 | Hs.124434 | ESTs | 3.01 | 3.70 |
| 421451 | AA291377 | Hs.50831 | ESTs | 2.99 | 14.95 |
| 426682 | AV660038 | Hs.2056 | UDP glycosyltransferase 1 family, polype | 2.97 | 2.10 |
| 402239 | | | Target Exon | 2.97 | 3.37 |
| 429345 | R11141 | Hs.199695 | hypothetical protein | 2.96 | 2.61 |
| 435904 | AF261655 | Hs.8910 | 1,2-alpha-mannosidase IC | 2.93 | 2.13 |
| 423961 | D13666 | Hs.136348 | periostin (OSF-2os) | 2.93 | 1.44 |
| 420923 | AF097021 | Hs.273321 | differentially expressed in hematopoieti | 2.92 | 0.95 |
| 436608 | AA628980 | | down syndrome critical region protein DS | 2.92 | 4.86 |
| 447343 | AA256641 | Hs.236894 | ESTs, Highly similar to S02392 alpha-2-m | 2.88 | 2.93 |
| 439223 | AW238299 | Hs.250618 | UL16 binding protein 2 | 2.88 | 2.15 |
| 401747 | | | Homo sapiens keratin 17 (KRT17) | 2.88 | 3.44 |
| 410102 | AW248508 | Hs.279727 | ESTs; homologue of PEM-3 [Ciona savignyi | 2.86 | 14.30 |
| 444444 | AI149332 | Hs.14855 | ESTs | 2.85 | 2.68 |
| 421100 | AW351839 | Hs.124660 | Homo sapiens cDNA: FLJ21763 fis, clone C | 2.84 | 2.46 |
| 431070 | AW408164 | Hs.249184 | transcription factor 19 (SC1) | 2.78 | 2.26 |
| 417389 | BE260964 | Hs.82045 | midkine (neurite growth-promoting factor | 2.77 | 2.34 |
| 442994 | AI026718 | Hs.16954 | ESTs | 2.75 | 2.82 |
| 444381 | BE387335 | Hs.283713 | ESTs, Weakly similar to S64054 hypotheti | 2.74 | 2.44 |
| 434487 | AF143867 | Hs.337588 | ESTs, Moderately similar to S65657 alpha | 2.72 | 3.37 |
| 417003 | AL038170 | Hs.80756 | betaine-homocysteine methyltransferase | 2.69 | 2.70 |
| 404440 | | | NM_021048: Homo sapiens melanoma antigen, | 2.69 | 13.45 |
| 400844 | | | NM_003105*: Homo sapiens sortilin-related | 2.69 | 13.45 |
| 426322 | J05068 | Hs.2012 | transcobalamin I (vitamin B12 binding pr | 2.69 | 1.36 |
| 431448 | AL137517 | Hs.306201 | hypothetical protein DKFZp564O1278 | 2.69 | 12.08 |
| 403381 | | | ENSP00000231844*: Ecotropic virus integra | 2.68 | 13.40 |
| 411248 | AA551538 | Hs.334605 | Home sapiens cDNA FLJ14408 fis, clone HE | 2.68 | 2.43 |
| 425206 | NM_002153 | Hs.155109 | hydroxysteroid (17-beta) dehydrogenase 2 | 2.67 | 2.68 |
| 435099 | AC004770 | Hs.4756 | flap structure-specific endonuclease 1 | 2.67 | 1.79 |
| 409361 | NM_005982 | Hs.54416 | sine oculis homeobox (Drosophila) homolo | 2.65 | 0.73 |
| 413281 | AA861271 | Hs.222024 | transcription factor BMAL2 | 2.65 | 2.23 |
| 446082 | AI274139 | Hs.156452 | ESTs | 2.65 | 2.65 |
| 422424 | AI186431 | Hs.296638 | prostate differentiation factor | 2.64 | 2.68 |
| 407839 | AA045144 | Hs.161566 | ESTs | 2.64 | 1.08 |
| 432441 | AW292425 | Hs.163484 | ESTs | 2.64 | 6.14 |
| 417312 | AW888411 | Hs.250811 | leukemia-associated phosphoprotein p18( | 2.64 | 1.31 |
| 430157 | BE348706 | Hs.278543 | ESTs | 2.63 | 2.58 |
| 436481 | AA379597 | Hs.5199 | HSPC150 protein similar to ubiquitin-con | 2.61 | 1.93 |
| 418686 | Z36830 | Hs.87268 | annexin A8 | 2.60 | 1.62 |
| 430486 | BE062109 | Hs.241551 | chloride channel, calcium activated, fam | 2.59 | 2.87 |
| 429276 | AF056085 | Hs.198612 | G protein-coupled receptor 51 | 2.57 | 3.89 |
| 439738 | BE246502 | Hs.9598 | sema domain, immunoglobulin domain (Ig), | 2.57 | 2.49 |
| 409632 | W74001 | Hs.55279 | serine (or cysteine) proteinase inhibito | 2.56 | 1.43 |
| 414812 | X72755 | Hs.77367 | monokine induced by gamma interferon | 2.54 | 3.10 |
| 451668 | Z43948 | Hs.326444 | cartilage acidic protein 1 | 2.51 | 3.60 |
| 421379 | Y15221 | Hs.103982 | small inducible cytokine subfamily B (Cy | 2.50 | 2.91 |
| 414416 | AW409985 | Hs.76084 | hypothetical protein MGC2721 | 2.49 | 1.78 |
| 429612 | AF062649 | Hs.252587 | pituitary tumor-transforming 1 | 2.49 | 1.80 |
| 416658 | U03272 | Hs.79432 | fibrillin 2 (congenital contractural ara | 2.49 | 3.46 |
| 437553 | AI829935 | Hs.130497 | ESTs, Weakly similar to MAT8_HUMAN CHLOR | 2.48 | 2.36 |
| 418941 | AA452970 | Hs.239527 | E1B-55 kDa-associated protein 5 | 2.46 | 2.33 |
| 414807 | AI738616 | Hs.77348 | hydroxyprostaglandin dehydrogenase 15-(N | 2.44 | 2.49 |
| 450983 | AA305384 | Hs.25740 | ERO1 (S. cerevisiae)-like | 2.43 | 1.42 |
| 407788 | BE514982 | Hs.38991 | S100 calcium-binding protein A2 | 2.41 | 2.05 |
| 449019 | AI949095 | Hs.67776 | ESTs, Weakly similar to T22341 hypotheti | 2.40 | 1.90 |
| 417366 | BE185289 | Hs.1076 | small proline-rich protein 1B (cornifin) | 2.40 | 0.49 |
| 420370 | Y13645 | Hs.97234 | uroplakin 2 | 2.39 | 3.81 |
| 408000 | L11690 | Hs.198689 | bullous pemphigoid antigen 1 (230/240 kD) | 2.38 | 1.45 |
| 406399 | | | NM_003122*: Homo sapiens serine protease | 2.36 | 4.20 |
| 435563 | AF210317 | Hs.95497 | solute carrier family 2 (facilitated glu | 2.31 | 4.05 |
| 442117 | AW664964 | Hs.128899 | ESTs; hypothetical protein for IMAGE: 447 | 2.31 | 1.17 |
| 436246 | AW450963 | Hs.119991 | ESTs | 2.30 | 11.50 |
| 433078 | AW015188 | Hs.121575 | Homo sapiens cDNA FLJ12231 fis, clone MA | 2.30 | 2.40 |
| 424012 | AW368377 | Hs.137569 | tumor protein 63 kDa with strong homolog | 2.29 | 1.89 |
| 411263 | BE297802 | Hs.69360 | kinesin-like 6 (mitotic centromere-assoc | 2.28 | 2.14 |
| 432829 | W60377 | Hs.57772 | ESTs | 2.28 | 4.85 |
| 415025 | AW207091 | Hs.72307 | ESTs | 2.28 | 11.40 |
| 436293 | AI601188 | Hs.120910 | ESTs | 2.27 | 3.80 |
| 415989 | AI267700 | | ESTs | 2.27 | 11.35 |
| 418067 | AI127958 | Hs.83393 | cystatin E/M | 2.25 | 1.54 |
| 436291 | BE568452 | Hs.344037 | protein regulator of cytokinesis 1 | 2.25 | 2.45 |
| 422278 | AF072873 | Hs.114218 | frizzled (Drosophila) homolog 6 | 2.25 | 1.55 |
| 428479 | Y00272 | Hs.334562 | cell division cycle 2, G1 to S and G2 to | 2.22 | 11.10 |
| 443247 | BE614387 | Hs.333893 | c-Myc target JPO1 | 2.21 | 1.32 |
| 424364 | AW383226 | Hs.201189 | ESTs, Weakly similar to G01763 atrophin- | 2.21 | 1.52 |
| 405932 | | | C15000305: gi\|3806122\|gb\|AAC69198.1\| (AFO | 2.20 | 1.55 |
| 419741 | NM_007019 | Hs.93002 | ubiquitin carrier protein E2-C | 2.19 | 1.99 |
| 423271 | W47225 | Hs.126256 | interleukin 1, beta | 2.19 | 2.01 |
| 402305 | | | C19000735*: gi\|4508027\|ref\|NP_003414.1\| z | 2.19 | 2.54 |
| 421064 | AI245432 | Hs.101382 | tumor necrosis factor, alpha-induced pro | 2.19 | 2.16 |
| 427747 | AW411425 | Hs.180655 | serine/threonine kinase 12 | 2.18 | 1.80 |
| 437181 | AI306615 | Hs.125343 | ESTs, Weakly similar to KIAA0758 protein | 2.17 | 10.85 |
| 431890 | X17033 | Hs.271986 | integrin, alpha 2 (CD49B, alpha 2 subuni | 2.17 | 2.14 |
| 433437 | U20536 | Hs.3280 | caspase 6, apoptosis-related cysteine pr | 2.16 | 1.37 |
| 407581 | R48402 | Hs.173508 | P3ECSL | 2.15 | 1.95 |
| 400845 | | | NM_003105*: Homo sapiens sortilin-related | 2.15 | 2.23 |
| 419359 | AL043202 | Hs.90073 | chromosome segregation 1 (yeast homolog) | 2.14 | 1.70 |
| 418526 | BE019020 | Hs.85838 | solute carrier family 16 (monocarboxylic | 2.13 | 1.83 |
| 448045 | AJ297436 | Hs.20166 | prostate stem cell antigen | 2.13 | 3.49 |
| 453459 | BE047032 | Hs.257789 | ESTs | 2.13 | 2.30 |
| 419183 | U60669 | Hs.89663 | cytochrome P450, subfamily XXIV (vitamin | 2.12 | 10.60 |
| 436251 | BE515065 | Hs.296585 | nucleolar protein (KKE/D repeat) | 2.11 | 1.89 |
| 445911 | AI985987 | Hs.145645 | ESTs, Moderately similar to ALU1_HUMAN A | 2.11 | 2.57 |
| 420876 | AA918425 | Hs.177744 | ESTs | 2.09 | 2.30 |
| 438817 | AI023799 | Hs.163242 | ESTs | 2.09 | 10.45 |
| 434293 | NM_004445 | Hs.3796 | EphB6 | 2.08 | 2.42 |
| 422765 | AW409701 | Hs.1578 | baculoviral IAP repeat-containing 5 (sur | 2.08 | 2.10 |
| 418216 | AA662240 | Hs.283099 | AF15q14 protein | 2.08 | 4.62 |
| 437915 | AI637993 | Hs.202312 | Homo sapiens clone N11 NTera2D1 teratoca | 2.08 | 10.40 |
| 409420 | Z15008 | Hs.54451 | laminin, gamma 2 (nicein (100 kD), kalini | 2.05 | 5.39 |
| 431441 | U81961 | Hs.2794 | sodium channel, nonvoltage-gated 1 alpha | 2.05 | 1.20 |
| 400773 | | | NM_003105*: Homo sapiens sortilin-related | 2.05 | 1.78 |
| 414987 | AA524394 | Hs.294022 | hypothetical protein FLJ14950 | 2.04 | 2.06 |
| 424687 | J05070 | Hs.151738 | matrix metalloproteinase 9 (gelatinase B | 2.04 | 1.70 |
| 444476 | AF020038 | Hs.11223 | isocitrate dehydrogenase 1 (NADP), solub | 2.03 | 2.26 |
| 447437 | U07225 | Hs.339 | purinergic receptor P2Y, (G-protein coupl | 2.02 | 1.73 |
| 445537 | AJ245671 | Hs.12844 | EGF-like-domain, multiple 6 | 2.02 | 2.93 |
| 407601 | AC002300 | Hs.37129 | sodium channel, nonvoltage-gated 1, beta | 2.02 | 1.55 |
| 441801 | AW242799 | Hs.86366 | ESTs | 2.01 | 10.05 |
| 439780 | AL109688 | | gb: Homo sapiens mRNA full length insert | 2.00 | 10.00 |
| 452732 | BE300078 | Hs.80449 | Homo sapiens , clone IMAGE: 3535294, mRNA, | 2.00 | 1.53 |
| 434876 | AF160477 | Hs.61460 | Ig superfamily receptor LNIR | 2.00 | 1.91 |
| 430152 | AB001325 | Hs.234642 | aquaporin 3 | 1.99 | 1.74 |
| 453134 | AA032211 | Hs.118493 | ESTs | 1.99 | 3.16 |
| 412719 | AW016610 | Hs.816 | ESTs | 1.99 | 0.34 |
| 442577 | AA292998 | Hs.163900 | ESTs | 1.99 | 3.09 |
| 409402 | AF208234 | Hs.695 | cystatin B (stefin B) | 1.98 | 1.50 |
| 414774 | X02419 | Hs.77274 | plasminogen activator, urokinase | 1.97 | 1.84 |
| 439318 | AW837046 | Hs.6527 | G protein-coupled receptor 56 | 1.95 | 1.42 |
| 447334 | AA515032 | Hs.91109 | ESTs | 1.95 | 2.53 |
| 432015 | AL157504 | Hs.159115 | Homo sapiens mRNA; cDNA DKFZp586O0724 (f | 1.94 | 9.70 |
| 429002 | AW248439 | Hs.2340 | junction plakoglobin | 1.94 | 1.57 |
| 410553 | AW016824 | Hs.255527 | hypothetical protein MGC14128 | 1.94 | 2.02 |
| 420783 | AI659838 | Hs.99923 | lectin, galactoside-binding, soluble, 7 | 1.93 | 0.56 |
| 407811 | AW190902 | Hs.40098 | cysteine knot superfamily 1, BMP antagon | 1.92 | 1.01 |
| 448988 | Y09763 | Hs.22785 | gamma-aminobutyric acid (GABA) A recepto | 1.92 | 1.47 |
| 400303 | AA242758 | Hs.79136 | LIV-1 protein, estrogen regulated | 1.92 | 1.59 |
| 414918 | AI219207 | Hs.72222 | hypothetical protein FLJ13459 | 1.92 | 2.77 |
| 424522 | AL134847 | Hs.149957 | ribosomal protein S6 kinase, 90 kD, polyp | 1.92 | 1.21 |
| 413278 | BE563085 | Hs.833 | interferon-stimulated protein, 15 kDa | 1.92 | 1.72 |
| 428928 | BE409838 | Hs.194657 | cadherin 1, type 1, E-cadherin (epitheli | 1.91 | 1.41 |
| 414595 | AA641726 | Hs.289015 | hypothetical protein MGC4171 | 1.90 | 1.61 |
| 400846 | | | sortilin-related receptor, L(DLR class) | 1.90 | 1.93 |
| 417409 | BE272506 | Hs.82109 | syndecan 1 | 1.89 | 1.75 |
| 444781 | NM_014400 | Hs.11950 | GPI-anchored metastasis-associated prote | 1.88 | 1.16 |
| 418867 | D31771 | Hs.89404 | msh (Drosophila) homeo box homolog 2 | 1.88 | 3.09 |
| 419092 | J05581 | Hs.89603 | mucin 1, transmembrane | 1.88 | 1.18 |
| 446673 | NM_016361 | Hs.15871 | LPAP for lysophosphatidic acid phosphata | 1.87 | 2.01 |
| 431347 | AI133461 | Hs.251664 | insulin-like growth factor 2 (somatomedi | 1.86 | 1.87 |
| 430168 | AW968343 | Hs.24255 | DKFZP434I1735 protein | 1.86 | 2.11 |
| 412115 | AK001763 | Hs.73239 | hypothetical protein FLJ10901 | 1.86 | 1.77 |
| 402901 | | | NM_025206*: Homo sapiens hypothetical pro | 1.85 | 2.35 |
| 449027 | AJ271216 | Hs.22880 | dipeptidylpeptidase III | 1.85 | 1.59 |
| 410418 | D31382 | Hs.63325 | transmembrane protease, serine 4 | 1.84 | 1.90 |
| 418870 | AF147204 | Hs.89414 | chemokine (C-X-C motif), receptor 4 (fus | 1.84 | 1.03 |
| 414732 | AW410976 | Hs.77152 | minichromesome maintenance deficient (S. | 1.84 | 1.54 |
| 432210 | AI567421 | Hs.273330 | Homo sapiens , clone IMAGE: 3544662, mRNA, | 1.83 | 1.74 |
| 452934 | AA581322 | Hs.4213 | hypothetical protein MGC16207 | 1.82 | 1.84 |
| 431630 | NM_002204 | Hs.265829 | integrin, alpha 3 (antigen CD49C, alpha | 1.82 | 1.83 |
| 427239 | BE270447 | Hs.174070 | ubiquitin carrier protein | 1.82 | 1.74 |
| 402424 | | | NM_024901: Homo sapiens hypothetical prot | 1.81 | 1.61 |
| 418068 | AW971155 | Hs.293902 | ESTs, Weakly similar to ISHUSS protein d | 1.81 | 3.67 |
| 431846 | BE019924 | Hs.271580 | uroplakin 1B | 1.80 | 4.11 |
| 410153 | BE311926 | Hs.15830 | hypothetical protein FLJ12691 | 1.80 | 9.00 |
| 408522 | AI541214 | Hs.46320 | Small proline-rich protein SPRK [human, | 1.80 | 1.02 |
| 428330 | L22524 | Hs.2256 | matrix metalloproteinase 7 (matrilysin, | 1.80 | 2.26 |
| 451541 | BE279383 | Hs.26557 | plakophilin 3 | 1.79 | 1.16 |
| 415786 | AW419196 | Hs.257924 | hypothetical protein FLJ13782 | 1.79 | 5.59 |
| 424905 | NM_002497 | Hs.153704 | NIMA (never in mitosis gene a)-related k | 1.79 | 8.95 |
| 425852 | AK001504 | Hs.159651 | death receptor 6, TNF superfamily member | 1.79 | 2.08 |
| 437852 | BE001836 | Hs.256897 | ESTs, Weakly similar to dJ365O12.1 [H. sa | 1.77 | 2.96 |
| 437044 | AL035864 | Hs.69517 | differentially expressed in Fanconi's an | 1.76 | 1.43 |
| 439606 | W79123 | Hs.58561 | G protein-coupled receptor 87 | 1.76 | 8.80 |
| 424098 | AF077374 | Hs.139322 | small proline-rich protein 3 | 1.76 | 0.57 |
| 430890 | X54232 | Hs.2699 | glypican 1 | 1.73 | 1.39 |
| 452862 | AW378065 | Hs.8687 | ESTs | 1.73 | 1.99 |
| 427335 | AA448542 | Hs.251677 | G antigen 7B | 1.73 | 8.65 |
| 425883 | AL137708 | Hs.161031 | Homo sapiens mRNA; cDNA DKFZp434K0322 (f | 1.72 | 2.07 |
| 414907 | X90725 | Hs.77597 | polo (Drosophia)-like kinase | 1.72 | 1.65 |
| 428484 | AF104032 | Hs.184601 | solute carrier family 7 (cationic amino | 1.72 | 1.03 |
| 453883 | AI638516 | Hs.347524 | cofactor required for Sp1 transcriptiona | 1.71 | 1.66 |
| 448993 | AI471630 | Hs.8127 | KIAA0144 gene product | 1.71 | 1.52 |
| 422406 | AF025441 | Hs.116206 | Opa-interacting protein 5 | 1.71 | 5.52 |
| 428664 | AK001666 | Hs.189095 | similar to SALL1 (sal (Drosophila)-like | 1.71 | 8.55 |
| 444342 | NM_014398 | Hs.10887 | similar to lysosome-associated membrane | 1.71 | 8.55 |
| 428227 | AA321649 | Hs.2248 | small inducible cytokine subfamily B (Cy | 1.71 | 8.55 |
| 424735 | U31875 | Hs.272499 | short-chain alcohol dehydrogenase family | 1.71 | 13.98 |
| 447532 | AK000614 | Hs.18791 | hypothetical protein FLJ20607 | 1.70 | 1.84 |
| 414053 | BE391635 | Hs.75725 | transgelin 2 | 1.69 | 1.51 |
| 447342 | AI199268 | Hs.19322 | Homo sapiens , Similar to RIKEN cDNA 2010 | 1.69 | 8.45 |
| 426050 | AF017307 | Hs.166096 | E74-like factor 3 (ets domain transcript | 1.69 | 1.60 |
| 448262 | AW880830 | Hs.186273 | ESTs | 1.67 | 2.07 |
| 452316 | AA298484 | Hs.61265 | ESTs, Moderately similar to G786_HUMAN P | 1.66 | 0.70 |
| 452240 | AI591147 | Hs.61232 | ESTs | 1.66 | 1.23 |
| 417151 | AA194055 | Hs.293858 | ESTs | 1.65 | 2.08 |
| 452461 | N78223 | Hs.108106 | transcription factor | 1.65 | 8.25 |
| 418462 | BE001596 | Hs.85266 | integrin, beta 4 | 1.65 | 1.78 |
| 417900 | BE250127 | Hs.82906 | CDC20 (cell division cycle 20, S. cerevi | 1.64 | 1.59 |
| 438746 | AI885815 | Hs.184727 | Human melanoma-associated antigen p97 (m | 1.64 | 1.13 |
| 423161 | AL049227 | Hs.124776 | downstream of cadherin 6 (by 3.3 kb) | 1.63 | 1.81 |
| 453968 | AA847843 | Hs.62711 | High mobility group (nonhistone chromoso | 1.62 | 1.51 |
| 402777 | | | C1002652*: gi\|544327\|sp\|Q04799\|FMO5_RABIT | 1.62 | 2.33 |
| 436569 | BE439539 | Hs.279837 | glutathione S-transferase M2 (muscle) | 1.62 | 2.18 |
| 417515 | L24203 | Hs.82237 | ataxia-telangiectasia group D-associated | 1.62 | 1.27 |
| 413385 | M34455 | Hs.840 | indoleamine-pyrrole 2,3 dioxygenase | 1.61 | 2.05 |
| 410407 | X66839 | Hs.63287 | carbonic anhydrase IX | 1.60 | 1.78 |
| 450635 | AW403954 | Hs.25237 | mesenchymal stem cell protein DSCD75 | 1.60 | 1.63 |
| 437016 | AU076916 | Hs.5398 | guanine monphosphate synthetase | 1.59 | 1.50 |
| 451982 | F13036 | Hs.27373 | Homo sapiens mRNA; cDNA DKFZp564O1763 (f | 1.58 | 1.92 |
| 422247 | U18244 | Hs.113602 | solute carrier family 1 (high affinity a | 1.57 | 1.79 |
| 408908 | BE296227 | Hs.250822 | serine/threonine kinase 15 | 1.56 | 7.80 |
| 433159 | AB035898 | Hs.150587 | kinesin-like protein 2 | 1.56 | 7.80 |
| 443211 | AI128388 | Hs.143655 | ESTs | 1.56 | 7.80 |
| 409893 | AW247090 | Hs.57101 | minichromosome maintenance deficient (S. | 1.55 | 1.44 |
| 426900 | AW163564 | Hs.142375 | ESTs | 1.54 | 1.93 |
| 421066 | AU076725 | Hs.101408 | branched chain aminotransferase 2, mitoc | 1.54 | 1.71 |
| 413804 | T64682 | | gb: yc48b02.r1 Stratagene liver (937224) | 1.53 | 1.55 |
| 418641 | BE243136 | Hs.86947 | a disintegrin and metalloproteinase doma | 1.53 | 1.59 |
| 444783 | AK001468 | Hs.62180 | anillin (Drosophila Scraps homolog), act | 1.52 | 7.60 |
| 414035 | Y00630 | Hs.75716 | serine (or cysteine) proteinase inhibito | 1.49 | 0.52 |
| 418543 | NM_005329 | Hs.85962 | hyaluronan synthase 3 | 1.48 | 1.54 |
| 429211 | AF052693 | Hs.198249 | gap junction protein, beta 5 (connexin 3 | 1.48 | 1.39 |
| 402260 | | | NM_001436*: Homo sapiens fibrillarin (FBL | 1.47 | 1.48 |
| 424264 | D80400 | Hs.239388 | Human DNA sequence from clone RP1-304B14 | 1.47 | 7.35 |
| 433091 | Y12642 | Hs.3185 | lymphocyte antigen 6 complex, locus D | 1.47 | 1.37 |
| 422164 | NM_014312 | Hs.112377 | cortic al thymocyte receptor (X. laevis | 1.46 | 1.80 |
| 444163 | AI126098 | | gb: qc54g07.x1 Soares_placenta_8to9weeks_{—} | 1.45 | 1.58 |
| 452304 | AA025386 | Hs.61311 | ESTs, Weakly similar to S10590 cysteine | 1.45 | 1.58 |
| 445182 | AW189787 | | ESTs | 1.43 | 7.15 |
| 408930 | AA146721 | Hs.334686 | hypothetical protein FLJ21588 | 1.43 | 1.53 |
| 429359 | W00482 | Hs.2399 | matrix metalloproteinase 14 (membrane-in | 1.43 | 1.34 |
| 406467 | | | Target Exon | 1.42 | 7.10 |
| 424244 | AV647184 | Hs.143601 | hypothetical protein hCLA-iso | 1.42 | 1.32 |
| 422094 | AF129535 | Hs.272027 | F-box only protein 5 | 1.41 | 4.55 |
| 431322 | AW970622 | | gb: EST382704 MAGE resequences, MAGK Homo | 1.39 | 10.36 |
| 401760 | | | Target Exon | 1.37 | 1.16 |
| 425247 | NM_005940 | Hs.155324 | matrix metalloproteinase 11 (stromelysin | 1.36 | 1.39 |
| 424420 | BE614743 | Hs.146688 | prostaglandin E synthase | 1.33 | 1.49 |
| 421817 | AF146074 | Hs.108660 | ATP-binding cassette, sub-family C (CFTR | 1.31 | 1.44 |
| 422119 | AI277829 | Hs.111862 | KIAA0590 gene product | 1.31 | 1.22 |
| 418729 | AB028449 | Hs.87889 | helicase-moi | 1.30 | 0.84 |
| 418399 | AF131781 | Hs.84753 | hypothetical protein FLJ12442 | 1.30 | 1.32 |
| 453028 | AB006532 | Hs.31442 | RecQ protein-like 4 | 1.29 | 1.42 |
| 431369 | BE184455 | Hs.251754 | secretory leukocyte protease inhibitor ( | 1.29 | 0.61 |
| 453321 | AI984381 | Hs.232521 | ESTs | 1.27 | 6.35 |
| 421478 | AI683243 | Hs.97258 | ESTs, Moderately similar to S29539 ribos | 1.26 | 6.30 |
| 425726 | AF085808 | Hs.159330 | uroplakin 3 | 1.26 | 2.26 |
| 406906 | Z25424 | | gb: H. sapiens protein-serine/threonine ki | 1.26 | 1.11 |
| 429413 | NM_014058 | Hs.201877 | DESC1 protein | 1.25 | 1.01 |
| 418678 | NM_001327 | Hs.87225 | cancer/testis antigen (NY-ESO-1) | 1.23 | 1.17 |
| 431840 | AA534908 | Hs.2860 | POU domain, class 5, transcription facto | 1.22 | 1.59 |
| 417433 | BE270266 | Hs.82128 | 5T4 oncofetal trophoblast glycoprotein | 1.20 | 1.38 |
| 422397 | AJ223366 | Hs.116051 | Homo sapiens cDNA: FLJ22495 fis, clone H | 1.19 | 1.23 |
| 403903 | | | C5001632*: gi\|10645308\|gb\|AAG21430.1\|AC00 | 1.19 | 1.98 |
| 425721 | AC002115 | Hs.159309 | uroplakin 1A | 1.17 | 2.30 |
| 413943 | AW294416 | Hs.144687 | Homo sapiens cDNA FLJ12981 fis, clone NT | 1.17 | 5.85 |
| 431515 | NM_012152 | Hs.258583 | endothelial differentiation; lysophospha | 1.17 | 1.39 |
| 443859 | NM_013409 | Hs.9914 | follistatin | 1.17 | 1.17 |
| 419743 | AW408762 | Hs.5957 | Homo sapiens clone 24416 mRNA sequence | 1.13 | 2.83 |
| 422330 | D30783 | Hs.115263 | epiregulin | 1.13 | 5.65 |
| 420281 | AI623693 | Hs.323494 | Predicted cation efflux pump | 1.11 | 5.55 |
| 424717 | H03754 | Hs.152213 | wingless-type MMTV integration site fami | 1.08 | 5.40 |
| 440304 | BE159984 | Hs.125395 | ESTs | 1.06 | 5.30 |
| 422170 | AI791949 | Hs.112432 | anti-Mullerian hormone | 1.06 | 1.55 |
| 417599 | AA204688 | Hs.62954 | ESTs | 1.05 | 1.02 |
| 411874 | AA096106 | Hs.20403 | ESTs | 1.04 | 7.20 |
| 449961 | AW265634 | Hs.133100 | ESTs | 1.03 | 0.65 |
| 418506 | AA084248 | Hs.85339 | G protein-coupled receptor 39 | 1.03 | 0.92 |
| 432239 | X81334 | Hs.2936 | matrix metalloproteinase 13 (collagenase | 1.02 | 5.10 |
| 453216 | AL137566 | Hs.32405 | Homo sapiens mRNA; cDNA DKFZp586G0321 (f | 1.02 | 0.60 |
| 423634 | AW959908 | Hs.1690 | heparin-binding growth factor binding pr | 1.00 | 0.05 |
| 423017 | AW178761 | Hs.227948 | serine (or cysteine) proteinase inhibito | 1.00 | 0.25 |
| 453365 | AA035211 | Hs.17404 | SOX7 SRY (sex determining region Y)-box | 1.00 | 0.25 |
| 439239 | AI031540 | Hs.235331 | ESTs | 1.00 | 0.27 |
| 450684 | AA872605 | Hs.25333 | interleukin 1 receptor, type II | 1.00 | 0.45 |
| 425650 | NM_001944 | Hs.1925 | desmoglein 3 (pemphigus vulgaris antigen | 1.00 | 0.72 |
| 404403 | | | Target Exon | 1.00 | 1.00 |
| 406974 | M57293 | | gb: Human parathyroid hormone-related pep | 1.00 | 1.00 |
| 410348 | AW182663 | Hs.95469 | ESTs | 1.00 | 1.00 |
| 412661 | N32860 | Hs.24611 | ESTs, Weakly similar to I54374 gene NF2 | 1.00 | 1.00 |
| 419121 | AA374372 | Hs.89626 | parathyoid hormone-like hormone | 1.00 | 1.00 |
| 426320 | W47595 | Hs.169300 | transforming growth factor, beta 2 | 1.00 | 1.00 |
| 426968 | U07616 | Hs.173034 | amphiphysin (Stiff-Mann syndrome with br | 1.00 | 1.00 |
| 432097 | X51730 | Hs.2905 | progesterone receptor | 1.00 | 1.00 |
| 452401 | NM_007115 | Hs.29352 | tumor necrosis factor, alpha-induced pro | 1.00 | 1.00 |
| 453389 | BE273648 | Hs.32963 | cadherin 6, type 2, K-cadherin (fetal ki | 1.00 | 1.00 |
| 419078 | M93119 | Hs.89584 | insulinoma-associated 1 | 1.00 | 1.25 |
| 430378 | Z29572 | Hs.2556 | tumor necrosis factor receptor superfami | 1.00 | 1.35 |
| 428182 | BE386042 | Hs.293317 | ESTs, Weakly similar to GGC1_HUMAN G ANT | 1.00 | 1.75 |
| 451844 | T61430 | | gb: yc06a03.s1 Stratagene lung (937210) H | 1.00 | 1.80 |
| 415178 | D80503 | Hs.322850 | ESTs | 1.00 | 2.20 |
| 410044 | BE566742 | Hs.58169 | highly expressed in cancer, rich in leuc | 1.00 | 2.25 |
| 425048 | H05468 | Hs.164502 | ESTs | 1.00 | 2.25 |
| 422956 | BE545072 | Hs.122579 | ECT2 protein (Epithelial cell transformi | 1.00 | 2.60 |
| 449448 | D60730 | Hs.57471 | ESTs | 1.00 | 2.70 |
| 417791 | AW965339 | Hs.111471 | ESTs | 1.00 | 2.95 |
| 421373 | AA608229 | Hs.167771 | ESTs | 1.00 | 3.00 |
| 427356 | AW023482 | Hs.97849 | ESTs | 1.00 | 3.15 |
| 421070 | AA283185 | Hs.19327 | ESTs | 1.00 | 3.25 |
| 415542 | R13474 | Hs.290263 | ESTs, Weakly similar to I38022 hypotheti | 1.00 | 3.35 |
| 429486 | AF155827 | Hs.203963 | hypothetical protein FLJ10339 | 1.00 | 3.55 |
| 402075 | | | ENSP00000251056*: Plasma membrane calcium | 1.00 | 3.95 |
| 419559 | Y07828 | Hs.91096 | ring finger protein | 1.00 | 4.00 |
| 416661 | AA634543 | Hs.79440 | IGF-II mRNA-binding protein 3 | 1.00 | 4.00 |
| 418738 | AW388633 | Hs.6682 | solute carrier family 7, (cationic amino | 1.00 | 4.35 |
| 412723 | AA648459 | Hs.335951 | hypothetical protein AF301222 | 1.00 | 4.40 |
| 404877 | | | NM_005365: Homo sapiens melanoma antigen, | 1.00 | 4.45 |
| 443054 | AI745185 | Hs.8939 | yes-associated protein 65 kDa | 1.00 | 4.45 |
| 403047 | | | NM_005656*: Homo sapiens transmembrane pr | 1.00 | 4.50 |
| 406434 | | | NM_030579*: Homo sapiens cytochrome b5 ou | 1.00 | 4.65 |
| 412530 | AA766268 | Hs.266273 | hypothetical protein FLJ13346 | 1.00 | 4.65 |
| 433365 | AF026944 | Hs.293797 | ESTs | 1.00 | 10.05 |
| 427666 | AI791495 | Hs.180142 | calmodulin-like skin protein (CLSP) | 0.99 | 0.60 |
| 429504 | X99133 | Hs.204238 | lipocalin 2 (oncogene 24p3) | 0.99 | 1.00 |
| 431474 | AL133990 | Hs.190642 | CEGP1 protein | 0.94 | 9.14 |
| 411880 | AW872477 | | gb: hm30f03.x1 NCI_CGAP_Thy4 Homo sapiens | 0.93 | 21.15 |
| 414221 | AW450979 | | gb: UI-H-BI3-ala-a-12-0-UI.s1 NCI_CGAP_Su | 0.91 | 2.60 |
| 444649 | AW207523 | Hs.197628 | ESTs | 0.89 | 11.15 |
| 456034 | AW450979 | | gb: UI-H-BI3-ala-a-12-0-UI.s1 NCI_CGAP_Su | 0.89 | 5.13 |
| 414521 | D28124 | Hs.76307 | neuroblastoma, suppression of tumorigeni | 0.84 | 0.85 |
| 439569 | AW602166 | Hs.222399 | CEGP1 protein | 0.84 | 2.42 |
| 432222 | AI204995 | | gb: an03c03.x1 Stratagene schizo brain S1 | 0.81 | 6.75 |
| 407846 | AA426202 | Hs.40403 | Cbp/p300-interacting transactivator, wit | 0.80 | 0.57 |
| 457292 | AI921270 | Hs.281462 | hypothetical protein FLJ14251 | 0.77 | 1.40 |
| 431089 | BE041395 | | ESTs, Weakly similar to unknown protein | 0.76 | 14.88 |
| 459702 | AI204995 | | gb: an03c03.x1 Stratagene schizo brain S1 | 0.74 | 11.03 |
| 424503 | NM_002205 | Hs.149609 | integrin, alpha 5 (fibronectin receptor, | 0.56 | 0.80 |
| 406964 | M21305 | | gb: Human alpha satellite and satellite 3 | 0.53 | 11.16 |
| 416225 | AA577730 | Hs.188684 | ESTs, Weakly similar to PC4259 ferritin | 0.48 | 3.01 |
| 400288 | X06256 | Hs.149609 | integrin, alpha 5 (fibronectin receptor, | 0.40 | 0.70 |
| 421218 | NM_000499 | Hs.72912 | cytochrome P450, subfamily I (aromatic c | 0.40 | 0.61 |

Pkey: Unique Eos probeset identifier number
ExAccn: Exemplar Accession number, Genbank accession number
UnigeneID: Unigene number
Unigene Title: Unigene gene title
R1: 90th percentile of bladder tumor Als divided by the 90th percentile of normal bladder sample Als
R2: 90th percentile of bladder tumor Als divided by the 90th percentile of normal body sample Als

TABLE 3B

| Pkey | CAT Number | Accession |
|---|---|---|
| 411880 | 1263110_1 | AW872477 BE088101 T05990 |
| 413804 | 1390710_1 | T64682 BE168190 BE168256 |
| 414221 | 142696_1 | AW450979 AA136653 AA136656 AW419381 AA984358 AA492073 BE168945 AA809054 AW238038 |
| | | BE011212 BE011359 BE011367 BE011368 BE011362 BE011215 BE011365 BE011363 |
| 415989 | 156454_1 | AI267700 AI720344 AA191424 AI023543 AI469633 AA172056 AW958465 AA172236 AW953397 |
| | | AA355086 |
| 431089 | 327825_1 | BE041395 AA491826 AA621946 AA715980 AA666102 |
| 431322 | 331543_1 | AW970622 AA503009 AA502998 AA502989 AA502805 T92188 |
| 432222 | 343347_1 | AI204995 AW827539 AW969908 AW440776 AA528756 |
| 436608 | 42361_3 | AA628980 AI126603 BE504035 |
| 439780 | 47673_1 | AL109688 R23665 R26578 |
| 444163 | 593658_1 | AI126098 AI184746 AI148521 |
| 445182 | 632151_1 | AW189787 AI215430 AW268499 AW205930 AI392907 BE093017 BE093019 BE093010 |
| 451844 | 888230_1 | T61430 AI820546 AI821336 |
| 456034 | 142696_1 | AW450979 AA136653 AA136656 AW419381 AA984358 AA492073 BE168945 AA809054 AW238038 |
| | | BE011212 BE011359 BE011367 BE011368 BE011362 BE011215 BE011365 BE011363 |

Pkey: Unique Eos probeset identifier number
CAT number: Gene cluster number
Accession: Genbank accession numbers

TABLE 3C

| Pkey | Ref | Strand | Nt_position |
|---|---|---|---|
| 400773 | 8131629 | Minus | 44116-44238, 48208-48321 |
| 400843 | 9188605 | Plus | 5863-5970, 7653-7784, 8892-9023, 9673-9807, 10634-10789, 15254-15403, 23827-23958 |
| 400844 | 9188605 | Plus | 24746-24872, 25035-25204 |
| 400845 | 9188605 | Plus | 34428-34612 |
| 400846 | 9188605 | Plus | 39310-39474 |
| 401093 | 8516137 | Minus | 22335-23166 |
| 401747 | 9789672 | Minus | 118596-118816, 119119-119244, 119609-119761, 120422-120990, 130161-130381, |
| | | | 130468-130593, 131097-131258, 131866-131932, 132451-132575, 133580-134011 |
| 401760 | 9929699 | Plus | 83126-83250, 85320-85540, 94719-95287 |
| 401780 | 7249190 | Minus | 28397-28617, 28920-29045, 29135-29296, 29411-29567, 29705-29787, 30224-30573 |
| 401781 | 7249190 | Minus | 83215-83435, 83531-83656, 83740-83901, 84237-84393, 84955-85037, 86290-86814 |
| 402075 | 8117407 | Plus | 121907-122035, 122804-122921, 124019-124161, 124455-124610, 125672-126076 |
| 402230 | 9966312 | Minus | 29782-29932 |
| 402239 | 7690131 | Plus | 38175-38304, 42133-42266 |
| 402260 | 3399665 | Minus | 113765-113910, 115653-115765, 116808-116940 |
| 402305 | 7328724 | Plus | 40832-41362 |
| 402424 | 9796344 | Minus | 64925-65073 |
| 402777 | 9588235 | Plus | 126786-126948 |
| 402901 | 8894222 | Minus | 175426-175667 |
| 403047 | 3540153 | Minus | 59793-59968 |
| 403381 | 9438267 | Minus | 26009-26178 |
| 403903 | 7710671 | Minus | 101165-102597 |
| 404403 | 7272157 | Minus | 72053-72238 |
| 404440 | 7528051 | Plus | 80430-81581 |
| 404875 | 9801324 | Plus | 96588-96732, 97722-97831 |
| 404877 | 1519284 | Plus | 1095-2107 |
| 404977 | 3738341 | Minus | 43081-43229 |
| 405033 | 7107731 | Minus | 142358-142546 |
| 405932 | 7767812 | Minus | 123525-123713 |
| 406081 | 9123861 | Minus | 38115-38691 |
| 406399 | 9256288 | Minus | 63448-63554 |
| 406434 | 9256651 | Minus | 17803-17931 |
| 406467 | 9795551 | Plus | 182212-182958 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 4A
Preferred diagnostics for bladder cancer

| Pkey | ExAccn | UnigeneID | Unigene Title | R1 | R2 | R3 |
|---|---|---|---|---|---|---|
| 423961 | D13666 | Hs.136348 | periostin (OSF-2os) | 11.22 | 1.40 | 1.44 |
| 446619 | AU076643 | Hs.313 | secreted phosphoprotein 1 (osteopontin, | 8.40 | 1.31 | 1.38 |
| 444381 | BE387335 | Hs.283713 | ESTs, Weakly similar to S64054 hypotheti | 7.88 | 2.06 | 2.44 |
| 408243 | Y00787 | Hs.624 | interleukin 8 | 7.54 | 2.86 | 4.85 |
| 413063 | AL035737 | Hs.75184 | chilinase 3-like 1 (cartilage glycoprote | 6.14 | 1.18 | 1.18 |
| 414020 | NM_002984 | Hs.75703 | small inducible cytokine A4 (homologous | 5.32 | 1.34 | 1.36 |
| 424247 | X14008 | Hs.234734 | Iysozyme (renal amyloidosis) | 5.27 | 0.61 | 0.57 |
| 418007 | M13509 | Hs.83169 | matrix metalloproteinase 1 (interstitial | 5.17 | 7.47 | 35.60 |
| 422158 | L10343 | Hs.112341 | protease inhibitor 3, skin-derived (SKAL | 4.30 | 1.75 | 1.54 |
| 446921 | AB012113 | Hs.16530 | small inducible cytokine subfamily A (Cy | 4.29 | 1.35 | 1.60 |
| 413324 | V00571 | Hs.75294 | corticotropin releasing hormone | 4.20 | 6.27 | 45.75 |
| 412429 | AV650262 | Hs.75765 | GRO2 oncogene | 4.00 | 0.94 | 0.93 |
| 406636 | L12064 | | gb: Homo sapiens (clone WR4.12VL)anti-th | 3.66 | 1.25 | 1.25 |
| 428330 | L22524 | Hs.2256 | matrix metalloproteinase 7 (matrilysin, | 3.58 | 2.07 | 2.26 |
| 406687 | M31126 | | matrix metalloproteinase 11 (stromelysin | 3.41 | 4.37 | 3.37 |
| 422550 | BE297626 | Hs.296049 | microfibrillar-associated protein 4 | 3.09 | 0.39 | 0.40 |
| 410867 | X63556 | Hs.750 | fibrillin 1 (Marfan syndrome) | 2.96 | 0.44 | 0.45 |
| 416658 | U03272 | Hs.79432 | fibrillin 2 (congenital contractural ara | 2.90 | 1.94 | 3.46 |
| 414812 | X72755 | Hs.77367 | monokine induced by gamma interferon | 2.80 | 1.67 | 3.10 |
| 423673 | BE003054 | Hs.1695 | matrix metalloproteinase 12 (macrophage | 2.77 | 5.62 | 26.07 |
| 421379 | Y15221 | Hs.103982 | small inducible cytokine subfamily B (Cy | 2.71 | 2.26 | 2.91 |
| 429500 | X78565 | Hs.289114 | hexabrachion (tenascin C, cytotactin) | 2.40 | 0.47 | 0.37 |
| 417849 | AW291587 | Hs.82733 | nidogen 2 | 2.34 | 0.88 | 0.86 |
| 400419 | AF084545 | | Target | 2.33 | 1.54 | 2.12 |
| 407811 | AW190902 | Hs.40098 | cysteine knot superfamily 1, BMP antagon | 2.10 | 1.01 | 1.01 |
| 400289 | X07820 | Hs.2258 | matrix metalloproteinase 10 (stromelysin | 1.99 | 3.19 | 17.50 |
| 414774 | X02419 | Hs.77274 | plasminogen activator, urokinase | 1.94 | 2.49 | 1.84 |
| 409420 | Z15008 | Hs.54451 | laminin, gamma 2 (nicein (100 kD), kalini | 1.94 | 2.02 | 5.39 |
| 428227 | AA321649 | Hs.2248 | small inducible cytokine subfamily B (Cy | 1.84 | 1.99 | 8.55 |
| 414476 | AA301867 | Hs.76224 | EGF-containing fibulin-like extracellula | 1.76 | 0.33 | 0.31 |
| 431639 | AK000680 | Hs.266175 | phosphoprotein associated with GEMs | 1.76 | 1.13 | 1.16 |
| 421958 | AA357185 | Hs.109918 | ras homolog gene family, member H | 1.75 | 1.33 | 1.27 |
| 425247 | NM_005940 | Hs.155324 | matrix metalloproteinase 11 (stromelysin | 1.58 | 2.42 | 1.39 |
| 444342 | NM_014398 | Hs.10887 | similar to lysosome-associated membrane | 1.57 | 2.02 | 8.55 |
| 421493 | BE300341 | Hs.104925 | ectodermal-neural cortex (with BTB-like | 1.55 | 1.57 | 1.55 |
| 407939 | W05608 | Hs.312679 | ESTs, Weakly similar to A49019 dynein he | 1.41 | 0.79 | 0.67 |
| 429344 | R94038 | Hs.199538 | inhibin, beta C | 1.36 | 1.39 | 1.34 |
| 402727 | | | NM_025065: Homo sapiens hypothetical prot | 1.34 | 1.34 | 1.56 |
| 412420 | AL035668 | Hs.73853 | bone morphogenetic protein 2 | 1.31 | 1.63 | 2.22 |
| 423217 | NM_000094 | Hs.1640 | collagen, type VII, alpha 1 (epidermolys | 1.27 | 2.00 | 1.67 |
| 424206 | NM_003734 | Hs.198241 | amine oxidase, copper containing 3 (vasc | 1.25 | 0.16 | 0.30 |
| 415138 | C18356 | Hs.295944 | tissue factor pathway inhibitor 2 | 1.23 | 1.70 | 4.34 |
| 424479 | AF064238 | Hs.149098 | smoothelin | 1.19 | 0.27 | 0.47 |
| 445537 | AJ245671 | Hs.12844 | EGF-like-domain, multiple 6 | 1.17 | 1.59 | 2.93 |
| 417079 | U65590 | Hs.81134 | interleukin 1 receptor antagonist | 1.16 | 0.82 | 0.80 |
| 421634 | AA437414 | Hs.106283 | hypothetical protein FLJ10262 | 1.16 | 1.05 | 1.05 |
| 439569 | AW602166 | Hs.222399 | CEGP1 protein | 1.15 | 2.01 | 2.42 |
| 431346 | AA371059 | Hs.251636 | ubiquitin specific protease 3 | 1.10 | 1.64 | 1.52 |
| 448901 | AK001021 | Hs.22505 | hypothetical protein FLJ10159 | 1.10 | 0.31 | 0.31 |
| 450983 | AA305384 | Hs.25740 | ERO1 (S. cerevisiae)-like | 1.03 | 1.51 | 1.42 |
| 422424 | AI186431 | Hs.296638 | prostate differentiation factor | 1.02 | 2.77 | 2.68 |
| 458781 | AI444821 | Hs.63085 | ESTs, Weakly similar to MPP3_HUMAN MAGUK | 1.00 | 1.64 | 5.45 |
| 445413 | AA151342 | Hs.12677 | CGI-147 protein | 1.00 | 1.51 | 5.20 |
| 432350 | NM_005865 | Hs.274407 | protease, serine, 16 (thymus) | 1.00 | 1.55 | 4.30 |
| 403106 | | | C8000064*: gi\|10432393\|emb\|CAC10283.1\| (A | 1.00 | 1.48 | 4.24 |
| 402075 | | | ENSP00000251056*: Plasma membrane calcium | 1.00 | 1.67 | 3.95 |
| 404860 | | | C1003394*: gi\|12314272\|emb\|CAC00591.1\| (A | 1.00 | 1.40 | 3.90 |
| 434037 | AF116601 | | WW domain-containing oxidoreductase | 1.00 | 1.58 | 3.70 |
| 405738 | | | CX000390*: gi\|6014646\|gb\|AAF01438.1\|AF187 | 1.00 | 1.36 | 2.95 |
| 427585 | D31152 | Hs.179729 | collagen, type X, alpha 1 (Schmid metaph | 1.00 | 1.45 | 1.60 |
| 439898 | AW505514 | Hs.209561 | KIAA1715 protein | 1.00 | 1.28 | 1.59 |
| 452567 | D87120 | Hs.29882 | predicted osteoblast protein | 1.00 | 1.10 | 1.31 |
| 401271 | | | C9000559*: gi\|12314195\|emb\|CAB99338.1\| (A | 1.00 | 2.12 | 1.00 |
| 411339 | BE164598 | Hs.274251 | hypothetical protein FLJ20375; KIAA1797 | 1.00 | 2.05 | 1.00 |
| 403005 | | | C21000027*: gi\|1817556\|dbj\|BAA13672.1\| (D | 1.00 | 1.89 | 1.00 |
| 431146 | Z83850 | | Human DNA sequence from PAC 82J11 and co | 1.00 | 1.89 | 1.00 |
| 434939 | AF161422 | Hs.306567 | Homo sapiens HSPC304 mRNA, partial cds | 1.00 | 1.86 | 1.00 |
| 431753 | X76029 | Hs.2841 | neuromedin U | 1.00 | 1.82 | 1.00 |
| 419121 | AA374372 | Hs.89626 | parathyroid hormone-like hormone | 1.00 | 1.69 | 1.00 |
| 435505 | AF200492 | Hs.211238 | interleukin-1 homolog 1 | 1.00 | 1.67 | 1.00 |
| 452401 | NM_007115 | Hs.29352 | tumor necrosis factor, alpha-induced pro | 1.00 | 1.67 | 1.00 |
| 406397 | | | C16001447*: gi\|12053709\|emb\|CAC20419.1\| ( | 1.00 | 1.66 | 1.00 |
| 404488 | | | NM_030958*: Homo sapiens organic anion tr | 1.00 | 1.56 | 1.00 |
| 441206 | BE552314 | Hs.131823 | ESTs, Weakly similar to TERA HUMAN [H. sa | 1.00 | 1.49 | 1.00 |
| 407853 | AA336797 | Hs.40499 | dickkopf (Xanopus laevis ) homolog 1 | 1.00 | 1.47 | 1.00 |
| 446119 | D29527 | Hs.290931 | ESTs | 1.00 | 1.47 | 1.00 |
| 406471 | | | Target Exon | 1.00 | 1.44 | 1.00 |
| 402110 | | | C18000178: gi\|11990779\|emb\|CAC19649.1\| (A | 1.00 | 1.42 | 1.00 |
| 407911 | AF104922 | Hs.41565 | growth differentiation factor 8 | 1.00 | 1.40 | 1.00 |
| 404829 | | | C1002937*: gi\|7499208\|pir∥T20993 hypothe | 1.00 | 1.37 | 1.00 |
| 421925 | S80310 | Hs.109620 | acidic epididymal glycoprotein-like 1 | 1.00 | 1.26 | 1.00 |
| 406076 | AL390179 | | Homo sapiens mRNA; cDNA DKFZp547P134 (fr | 1.00 | 1.19 | 1.00 |
| 458622 | AA972412 | Hs.13755 | f-box and WD-40 domain protein 2 | 1.00 | 1.09 | 1.00 |
| 416018 | AW138239 | Hs.78977 | proprotein convertase subtilisin/kexin t | 1.00 | 1.04 | 1.00 |
| 409357 | M73628 | Hs.54415 | casein, kappa | 1.00 | 1.03 | 1.00 |
| 436684 | AW976319 | Hs.94806 | ATP-binding cassette, sub-family A (ABC1 | 1.00 | 0.84 | 0.84 |
| 436178 | BE152396 | Hs.21590 | hypothetical protein DKFZp564O0523 | 1.00 | 0.91 | 0.80 |
| 402522 | | | C1000568*: gi\|12697965\|dbj\|BAB21801.1\| (A | 1.00 | 0.80 | 0.67 |
| 405735 | | | ENSP00000252164*: KIAA1578 protein (Fragm | 1.00 | 0.86 | 0.56 |
| 401905 | | | ENSP00000252232*: Sterol regulatory eleme | 1.00 | 0.65 | 0.52 |
| 404152 | | | C6000931*: gi\|9558454\|dbj\|BAB03398.1\| (AB | 1.00 | 0.58 | 0.51 |
| 418693 | AI750878 | Hs.87409 | thrombospondin 1 | 1.00 | 0.85 | 0.51 |
| 451375 | AI792066 | Hs.283902 | Homo sapiens BAC clone RP11-481J13 from | 1.00 | 0.46 | 0.38 |
| 430132 | AA204686 | Hs.234149 | hypothetical protein FLJ20647 | 1.00 | 0.84 | 0.33 |
| 456983 | AI081687 | Hs.11355 | thymopoietin | 1.00 | 0.61 | 0.29 |
| 438681 | AW384815 | Hs.149208 | KIAA1555 protein | 1.00 | 0.60 | 0.28 |
| 409038 | T97490 | Hs.50002 | small inducible cytokine subfamily A (Cy | 1.00 | 0.39 | 0.19 |
| 409196 | NM_001874 | Hs.334873 | carboxypeptidase M | 1.00 | 0.43 | 0.13 |
| 410023 | AB017169 | Hs.57929 | slit (Drosophila) homolog 3 | 1.00 | 0.30 | 0.12 |
| 420674 | NM_000055 | Hs.1327 | butyrylcholinesterase | 1.00 | 0.30 | 0.08 |
| 415165 | AW887604 | Hs.78065 | complement component 7 | 1.00 | 0.08 | 0.06 |
| 425545 | N98529 | Hs.158295 | Homo sapiens , clone MGC: 12401, mRNA, com | 1.00 | 0.10 | 0.01 |
| 448256 | BE614149 | Hs.20814 | CGI-27 protein | 0.96 | 1.32 | 1.55 |
| 417389 | BE260964 | Hs.82045 | midkine (neurite growth-promoting factor | 0.95 | 3.15 | 2.34 |
| 403214 | | | NM_016232*: Homo sapiens interleukin 1 re | 0.94 | 1.63 | 2.51 |
| 414799 | AI752416 | Hs.77326 | insulin-like growth factor binding prote | 0.92 | 1.87 | 1.60 |
| 406665 | U22961 | Hs.184411 | albumin | 0.92 | 1.09 | 1.03 |
| 401519 | | | C15000476*: gi\|12737279\|ref\|XP_012163.1\| | 0.88 | 1.46 | 3.44 |
| 417501 | AL041219 | Hs.82222 | sema domain, immunoglobulin domain (Ig), | 0.87 | 0.41 | 0.50 |
| 409632 | W74001 | Hs.55279 | serine (or cysteine) proteinase inhibito | 0.85 | 1.38 | 1.43 |
| 405494 | | | C2001837*: gi\|12697903\|dbj\|BAB21770.1\| (A | 0.83 | 1.46 | 4.65 |
| 444171 | AB018249 | Hs.10458 | small inducible cytokine subfamily A (Cy | 0.80 | 0.91 | 0.91 |
| 439706 | AW872527 | Hs.59761 | ESTs, Weakly similar to DAP1_HUMAN DEATH | 0.79 | 0.58 | 0.43 |
| 436396 | AI683487 | Hs.152213 | wingless-type MMTV integration site fami | 0.77 | 1.47 | 2.37 |
| 426716 | NM_006379 | Hs.171921 | sema domain, immunoglobulin domain (Ig), | 0.75 | 1.13 | 1.18 |
| 431347 | AI133461 | Hs.251664 | insulin-like growth factor 2 (somatomedi | 0.68 | 2.61 | 1.87 |
| 413753 | U17760 | Hs.75517 | laminin, beta 3 (nicein (125 kD), kalinin | 0.68 | 2.70 | 4.96 |
| 426322 | J05068 | Hs.2012 | transcobalamin I (vitamin B12 binding pr | 0.67 | 1.50 | 1.36 |
| 426514 | BE616633 | Hs.170195 | bone morphogenetic protein 7 (osteogenic | 0.56 | 2.05 | 2.46 |
| 422282 | AF019225 | Hs.114309 | apolipoprotein L | 0.55 | 3.91 | 3.92 |
| 409757 | NM_001898 | Hs.123114 | cystatin SN | 0.53 | 2.72 | 2.93 |
| 427450 | AB014526 | Hs.178121 | KIAA0626 gene product | 0.52 | 1.34 | 1.97 |
| 414555 | N98569 | Hs.76422 | phospholipase A2, group IIA (platelets, | 0.50 | 1.04 | 1.05 |
| 423774 | L39064 | Hs.1702 | interleukin 9 receptor | 0.49 | 2.81 | 6.46 |
| 404977 | | | Insulin-like growth factor 2 (somatomedi | 0.28 | 5.78 | 5.17 |
| 428336 | AA503115 | Hs.183752 | microseminoprotein, beta- | 0.21 | 1.47 | 1.56 |
| 451668 | Z43948 | Hs.326444 | cartilage acidic protein 1 | 0.18 | 4.05 | 3.60 |
| 428651 | AF196478 | Hs.188401 | annexin A10 | 0.17 | 5.14 | 27.75 |
| 421110 | AJ250717 | Hs.1355 | cathepsin E | 0.12 | 5.49 | 45.35 |

Pkey: Unique Eos probeset identifier number
ExAccn: Exemplar Accession number, Genbank accession number
UnigeneID: Unigene number
Unigene Title: Unigene gene title
R1: 80th percentile of muscle-invasive bladder tumor (stage T2-T4) Als divided by the 80th percentile of exophytic non-invasive carcinoma (stage Ta) Ais
R2 90th percentile of bladder tumor Als minus background divided by 90th percentile of normal body sample Als minus background, where background equals the 15th percentile of all sample Als
R3 90th percentile of bladder tumor Als divided by the 90th percentile of normal body sample Als

TABLE 4B

| Pkey | CAT Number | Accession |
|---|---|---|
| 431146 | 32854_1 | Z83850 AA459717 AW965384 AA333635 |
| 434037 | 37918_1 | AF116601 AI110691 AF063566 |

Pkey: Unique Eos probeset identifier number
CAT number: Gene cluster number
Accession: Genbank accession numbers

TABLE 4C

| Pkey | Ref | Strand | Nt_position |
|---|---|---|---|
| 401271 | 9797373 | Minus | 61292-61911 |
| 401519 | 6649315 | Plus | 157315-157950 |
| 401905 | 8671966 | Plus | 153965-154441, 156599-156819 |
| 402075 | 8117407 | Plus | 121907-122035, 122804-122921, |
| | | | 124019-124161, 124455-124610, |
| | | | 125672-126076 |
| 402110 | 8131678 | Minus | 173889-174062 |
| 402522 | 9798493 | Plus | 20605-20731 |
| 402727 | 9211324 | Plus | 54596-54777 |
| 403005 | 5791501 | Minus | 16945-17053, 20018-20403 |
| 403106 | 7331404 | Plus | 77162-77350, 81338-81511 |
| 403214 | 7630945 | Minus | 76723-77027, 79317-79484 |
| 404152 | 9884757 | Plus | 41111-41281, 45495-45716, |
| | | | 47801-47910 |
| 404488 | 8113286 | Minus | 64835-64994 |
| 404829 | 6624702 | Minus | 4913-5093, 7310-7469, 9472-9621, |
| | | | 9951-10082 |
| 404860 | 8979555 | Plus | 65852-66081 |
| 404977 | 3738341 | Minus | 43081-43229 |
| 405494 | 8050952 | Minus | 70284-70518 |
| 405735 | 9931101 | Minus | 29854-29976 |
| 405738 | 9943998 | Plus | 44370-45410 |
| 406076 | 9123123 | Plus | 89972-90319 |
| 406397 | 9256243 | Minus | 127317-127454 |
| 406471 | 9795566 | Plus | 87383-87589 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 5A
Genes upregulated in bladder cancer

| Pkey | ExAccn | UnigeneID | Unigene Title | R1 |
|---|---|---|---|---|
| 459702 | AI204995 | | gb: an03c03.x1 Stratagene schizo brain S1 | 11.03 |
| 437915 | AI637993 | Hs.202312 | Homo sapiens clone N11 NTera2D1 teratoca | 10.40 |
| 404917 | | | Target Exon | 9.65 |
| 401066 | | | C11000517*: gi\|7293105\|gb\|AAF48490.1\| (AE | 9.00 |
| 447475 | AI380797 | Hs.158992 | ESTs | 8.92 |
| 427335 | AA448542 | Hs.251677 | G antigen 7B | 8.65 |
| 450061 | AI797034 | Hs.346238 | ESTs | 8.35 |
| 401335 | | | Target Exon | 7.95 |
| 424264 | D80400 | Hs.239388 | Human DNA sequence from clone RP1-304B14 | 7.35 |
| 409041 | AB033025 | Hs.50081 | Hypothetical protein, XP_051860 (KIAA119 | 7.20 |
| 436608 | AA628980 | | down syndrome critical region protein DS | 4.86 |
| 451950 | AW292317 | Hs.213307 | ESTs | 4.45 |
| 406542 | | | C19000728*: gi\|12585552\|sp\|Q9Y2Q1\|Z257_HU | 3.73 |
| 437931 | AI249468 | Hs.124434 | ESTs | 3.70 |
| 443133 | AI033878 | Hs.41379 | ESTs | 3.60 |
| 434487 | AF143867 | Hs.337588 | ESTs, Moderately similar to S65657 alpha | 3.37 |
| 402239 | | | Target Exon | 3.37 |
| 443162 | T49951 | Hs.9029 | DKFZP434G032 protein | 3.21 |
| 403383 | | | Target Exon | 3.13 |
| 438315 | R56795 | Hs.82419 | ESTs | 3.04 |
| 452827 | AI571835 | Hs.55468 | ESTs | 3.01 |
| 416225 | AA577730 | Hs.188684 | ESTs, Weakly similar to PC4259 ferritin | 3.01 |
| 402948 | | | NM_025206: Homo sapiens hypothetical prot | 2.91 |
| 429983 | W92620 | Hs.260855 | ESTs | 2.84 |
| 429238 | NM_002849 | Hs.198288 | protein tyrosine phosphatase, receptor t | 2.78 |
| 444371 | BE540274 | Hs.239 | forkhead box M1 | 2.75 |
| 417003 | AL038170 | Hs.80756 | betaine-homocysteine methyltransferase | 2.70 |
| 414906 | AA157911 | Hs.72200 | ESTs | 2.70 |
| 425206 | NM_002153 | Hs.155109 | hydroxysteroid (17-beta) dehydrogenase 2 | 2.68 |
| 422283 | AW411307 | Hs.114311 | CDC45 (cell division cycle 45, S. cerevis | 2.62 |
| 429345 | R11141 | Hs.199695 | hypothetical protein | 2.61 |
| 414221 | AW450979 | | gb: UI-H-BI3-ala-a-12-0-UI.s1 NCI_CGAP_Su | 2.60 |
| 402305 | | | C19000735*: gi\|4508027\|ref\|NP_003414.1\| z | 2.54 |
| 432842 | AW674093 | Hs.334822 | hypothetical protein MGC4485 | 2.51 |
| 427719 | AI393122 | Hs.134726 | ESTs | 2.51 |
| 455797 | BE091833 | | gb: IL2-BT0731-260400-076-F04 BT0731 Homo | 2.50 |
| 414807 | AI738616 | Hs.77348 | hydroxyprostaglandin dehydrogenase 15-(N | 2.49 |
| 456967 | AW004056 | Hs.168357 | T-box 2 | 2.49 |
| 406387 | | | Target Exon | 2.48 |
| 417997 | AA418189 | Hs.23017 | Homo sapiens cDNA: FLJ22747 fis, clone K | 2.48 |
| 415752 | BE314524 | Hs.78776 | putative transmembrane protein | 2.46 |
| 411248 | AA551538 | Hs.334605 | Homo sapiens cDNA FLJ14408 fis, clone HE | 2.43 |
| 434293 | NM_004445 | Hs.3796 | EphB6 | 2.42 |
| 433078 | AW015188 | Hs.121575 | Homo sapiens cDNA FLJ12231 fis, clone MA | 2.40 |
| 425997 | AK000086 | Hs.165948 | hypothetical protein FLJ20079 | 2.38 |
| 418322 | AA284166 | Hs.84113 | cyclin-dependent kinase inhibitor 3 (CDK | 2.35 |
| 452012 | AA307703 | Hs.279766 | kinesin family member 4A | 2.34 |
| 445600 | AF034803 | Hs.12953 | PTPRF interacting protein, binding prote | 2.33 |
| 418941 | AA452970 | Hs.239527 | E1B-55 kDa-associated protein 5 | 2.33 |
| 454609 | AW810204 | | gb: MR4-ST0125-021199-017-d08 ST0125 Homo | 2.28 |
| 444476 | AF020038 | Hs.11223 | isocitrate dehydrogenase 1 (NADP), solub | 2.26 |
| 420005 | AW271106 | Hs.133294 | ESTs | 2.22 |
| 439826 | NM_014965 | Hs.6705 | KIAA1042 protein | 2.22 |
| 405531 | | | Target Exon | 2.21 |
| 436569 | BE439539 | Hs.279837 | glutathione S-transferase M2 (muscle) | 2.18 |
| 404394 | | | ENSP00000241075: TRRAP PROTEIN. | 2.17 |
| 427479 | BE410092 | Hs.178471 | KIAA0798 gene product | 2.17 |
| 435904 | AF261655 | Hs.8910 | 1,2-alpha-mannosidase IC | 2.13 |
| 431620 | AA126109 | Hs.264981 | 2′-5′-oligoadenylate synthetase 2 (69-71 | 2.12 |
| 426682 | AV660038 | Hs.2056 | UDP glycosyltransferase 1 family, polype | 2.10 |
| 422765 | AW409701 | Hs.1578 | baculoviral lAP repeat-containing 5 (sur | 2.10 |
| 451385 | AA017656 | | gb: ze39h01.r1 Soares retina N2b4HR Homo | 2.09 |
| 403477 | | | C3002160*: gi\|7662420\|ref\|NP_055738.1\| KI | 2.09 |
| 417151 | AA194055 | Hs.293858 | ESTs | 2.08 |
| 448262 | AW880830 | Hs.186273 | ESTs | 2.07 |
| 415192 | D17793 | Hs.78183 | aldo-keto reductase family 1, member C3 | 2.04 |
| 402994 | | | NM_002463*: Homo sapiens myxovirus (influ | 2.04 |
| 426053 | U68105 | Hs.172182 | poly(A)-binding protein, cytoplasmic 1 | 2.02 |
| 423271 | W47225 | Hs.126256 | interleukin 1, beta | 2.01 |
| 419741 | NM_007019 | Hs.93002 | ubiquitin carrier protein E2-C | 1.99 |
| 407581 | R48402 | Hs.173508 | P3ECSL | 1.95 |
| 410197 | NM_005518 | Hs.59889 | 3-hydroxy-3-methylglutaryl-Coenzyme A sy | 1.95 |
| 427122 | AW057736 | Hs.323910 | HER2 receptor tyrosine kinase (c-erb-b2, | 1.93 |
| 436481 | AA379597 | Hs.5199 | HSPC150 protein similar to ubiquitin-con | 1.93 |
| 436251 | BE515065 | Hs.296585 | nucleolar protein (KKE/D repeat) | 1.89 |
| 401961 | | | NM_021626: Homo sapiens serine carboxypep | 1.86 |
| 434042 | AI589941 | Hs.8254 | Home sapiens, Similar to tumor different | 1.85 |
| 447532 | AK000614 | Hs.18791 | hypothetical protein FLJ20607 | 1.84 |
| 418526 | BE019020 | Hs.85838 | solute carrier family 16 (monocarboxylic | 1.83 |
| 429612 | AF062649 | Hs.252587 | pituitary tumor-transforming 1 | 1.80 |
| 422164 | NM_014312 | Hs.112377 | cortic al thymocyte receptor (X. laevis | 1.80 |
| 422247 | U18244 | Hs.113602 | solute carrier family 1 (high affinity a | 1.79 |
| 410407 | X66839 | Hs.63287 | carbonic anhydrase IX | 1.78 |
| 412115 | AK001763 | Hs.73239 | hypothetical protein FLJ10901 | 1.77 |
| 414809 | AI434699 | Hs.77356 | transferrin receptor (p90, CD71) | 1.75 |
| 432210 | AI567421 | Hs.273330 | Homo sapiens, clone IMAGE: 3544662, mRNA, | 1.74 |
| 427239 | BE270447 | Hs.174070 | ubiquitin carrier protein | 1.74 |
| 459198 | AI086347 | Hs.151138 | ESTs | 1.74 |
| 421066 | AU076725 | Hs.101408 | branched chain aminotransferase 2, mitoc | 1.71 |
| 424687 | J05070 | Hs.151738 | matrix metalloproteinase 9 (gelatinase B | 1.70 |
| 450663 | H43540 | Hs.25292 | ribonuclease HI, large subunit | 1.70 |
| 417324 | AW265494 | | ESTs | 1.67 |
| 453883 | AI638516 | Hs.347524 | cofactor required for Sp1 transcriptiona | 1.66 |
| 428000 | R35145 | Hs.291904 | accessory proteins BAP31/BAP29 | 1.65 |
| 450635 | AW403954 | Hs.25237 | mesenchymal stem cell protein DSCD75 | 1.63 |
| 423397 | NM_001838 | Hs.1652 | chemokine (C-C motif) receptor 7 | 1.62 |
| 415440 | D83782 | Hs.78442 | SREBP CLEAVAGE-ACTIVATING PROTEIN | 1.62 |
| 428028 | U52112 | Hs.182018 | interleukin-1 receptor-associated kinase | 1.62 |
| 426783 | Z19084 | Hs.172210 | MUF1 protein | 1.62 |
| 445937 | AI452943 | Hs.321231 | UDP-Gal: betaGlcNAc beta 1,4-galactosylt | 1.61 |
| 445462 | AA378776 | Hs.288649 | hypothetical protein MGC3077 | 1.60 |
| 400965 | | | C11002190*: gi\|12737279\|ref\|XP_012163.1\| | 1.59 |
| 432269 | NM_002447 | Hs.2942 | macrophage stimulating 1 receptor (c-met | 1.59 |
| 429578 | AI969028 | Hs.99389 | ESTs | 1.59 |
| 449027 | AJ271216 | Hs.22880 | dipeptidylpeptidase III | 1.59 |
| 431840 | AA534908 | Hs.2860 | POU domain, class 5, transcription facto | 1.59 |
| 417900 | BE250127 | Hs.82906 | CDC20 (cell division cycle 20, S. cerevi | 1.59 |
| 429002 | AW248439 | Hs.2340 | junction plakoglobin | 1.57 |
| 442410 | AW996503 | Hs.197680 | ESTs | 1.56 |
| 407601 | AC002300 | Hs.37129 | sodium channel, nonvoltage-gated 1, beta | 1.55 |
| 418543 | NM_005329 | Hs.85962 | hyaluronan synthase 3 | 1.54 |
| 424611 | NM_001421 | Hs.151139 | E74-like factor 4 (ets domain transcript | 1.54 |
| 414732 | AW410976 | Hs.77152 | minichromosome maintenance deficient (S. | 1.54 |
| 408930 | AA146721 | Hs.334686 | hypothetical protein FLJ21588 | 1.53 |
| 448993 | AI471630 | Hs.8127 | KIAA0144 gene product | 1.52 |
| 414053 | BE391635 | Hs.75725 | transgelin 2 | 1.51 |
| 433662 | W07162 | Hs.150826 | CATX-8 protein | 1.50 |
| 432562 | BE531048 | Hs.278422 | DKFZP586G1122 protein | 1.50 |
| 402260 | | | NM_001436*: Homo sapiens fibrillarin (FBL | 1.48 |
| 426127 | L36983 | Hs.167013 | dynamin 2 | 1.48 |
| 427557 | NM_002659 | Hs.179657 | plasminogen activator, urokinase recepto | 1.48 |
| 418026 | BE379727 | Hs.83213 | fatty acid binding protein 4, adipocyte | 1.47 |
| 418960 | NM_004494 | Hs.89525 | hepatoma-derived growth factor (high-mob | 1.46 |
| 428293 | BE250944 | Hs.183556 | solute carrier family 1 (neutral amino a | 1.46 |
| 432344 | AI476474 | Hs.248156 | ESTs | 1.46 |
| 453449 | W16752 | Hs.32981 | sema domain, immunoglobulin domain (lg), | 1.46 |
| 450690 | AA296696 | Hs.333418 | FXYD domain-containing ion transport reg | 1.46 |
| 441940 | AW298115 | Hs.128152 | ESTs | 1.45 |
| 409893 | AW247090 | Hs.57101 | minichromosome maintenance deficient (S. | 1.44 |
| 439318 | AW837046 | Hs.6527 | G protein-coupled receptor 56 | 1.42 |
| 422565 | BE259035 | Hs.118400 | singed (Drosophila)-like (sea urchin fas | 1.41 |
| 428928 | BE409838 | Hs.194657 | cadherin 1, type 1, E-cadherin (epitheli | 1.41 |
| 445417 | AK001058 | Hs.12680 | Homo sapiens cDNA FLJ10196 fis, clone HE | 1.39 |
| 441565 | AW953575 | Hs.303125 | p53-induced protein PIGPC1 | 1.37 |
| 439180 | AI393742 | Hs.199067 | v-erb-b2 avian erythroblastic leukemia v | 1.35 |
| 418399 | AF131781 | Hs.84753 | hypothetical protein FLJ12442 | 1.32 |
| 432636 | AA340864 | Hs.278562 | claudin 7 | 1.32 |
| 439053 | BE244588 | Hs.6456 | chaperonin containing TCP1, subunit 2 (b | 1.32 |
| 413762 | AW411479 | Hs.848 | FK506-binding protein 4 (59 kD) | 1.31 |
| 453914 | NM_000507 | Hs.574 | fructose-1,6-bisphosphatase 1 | 1.28 |
| 430056 | X97548 | Hs.228059 | KRAB-asoociated protein 1 | 1.24 |
| 451524 | AK001466 | Hs.26516 | hypothetical protein FLJ10604 | 1.23 |
| 431441 | U81961 | Hs.2794 | sodium channel, nonvoltage-gated 1 alpha | 1.20 |
| 439863 | BE547830 | Hs.9408 | paired immunoglobulin-like receptor beta | 1.19 |
| 451541 | BE279383 | Hs.26557 | plakophilin 3 | 1.16 |
| 406906 | Z25424 | | gb: H. sapiens protein-serine/threonine ki | 1.11 |
| 429504 | X99133 | Hs.204238 | lipocalin 2 (oncogene 24p3) | 1.00 |
| 414002 | NM_006732 | Hs.75678 | FBJ murine osteosarcoma viral oncogene h | 0.86 |
| 431369 | BE184455 | Hs.251754 | secretory leukocyte protease inhibitor ( | 0.61 |

Pkey: Unique Eos probeset identifier number
ExAccn: Exemplar Accession number, Genbank accession number
UnigeneID: Unigene number
Unigene Title: Unigene gene title
R1 90th percentile of bladder tumor Als divided by the 90th percentile of normal body sample Als

TABLE 5B

| Pkey | CAT Number | Accession |
|---|---|---|
| 414221 | 142696_1 | AW450979 AA136653 AA136656 AW419381 |
| | | AA984358 AA492073 BE168945 AA809054 |
| | | AW238038 BE011212 BE011359 BE011367 |
| | | BE011368 BE011362 BE011215 |
| | | BE011365 BE011363 |
| 417324 | 166714_1 | AW265494 AA455904 AA195677 AW265432 |
| | | AW991605 AA456370 |
| 436608 | 42361_3 | AA628980 AI126603 BE504035 |
| 451385 | 86787_1 | AA017656 AA017374 AA019761 |
| 454609 | 1226517_1 | AW810204 AW810555 AW810196 |
| | | AW810619 AW810507 |
| 455797 | 1366826_1 | BE091833 BE091874 BE091871 |

Pkey: Unique Eos probeset identifier number
CAT number: Gene cluster number
Accession: Genbank accession numbers

TABLE 5C

| Pkey | Ref | Strand | Nt_position |
|---|---|---|---|
| 400965 | 7770576 | Minus | 173043-173564 |
| 401066 | 8217436 | Plus | 71448-71574 |
| 401335 | 9884881 | Plus | 15736-16352 |
| 401961 | 4581193 | Minus | 124054-124209 |
| 402239 | 7690131 | Plus | 38175-38304, 42133-42266 |
| 402260 | 3399665 | Minus | 113765-113910, 115653-115765, |
| | | | 116808-116940 |
| 402305 | 7328724 | Plus | 40832-41362 |
| 402948 | 9368458 | Minus | 143456-143626, 143808-143935 |
| 402994 | 2996643 | Minus | 4727-4969 |
| 403383 | 9438267 | Minus | 119837-121197 |
| 403477 | 9958251 | Plus | 111834-112008 |
| 404394 | 3135305 | Minus | 37121-37205, 37491-37762, |
| | | | 41053-41140, 41322-41593, |
| | | | 41773-41919 |
| 404917 | 7341851 | Plus | 49330-49498 |
| 405531 | 9665194 | Plus | 35602-35803 |
| 406387 | 9256180 | Plus | 116229-116371, 117512-117651 |
| 406542 | 7711499 | Plus | 117335-118473 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 6A
Genes upregulated in bladder cancer

| Pkey | ExAccn | UnigeneID | Unigene Title | R1 | R2 |
|---|---|---|---|---|---|
| 439926 | AW014875 | Hs.137007 | ESTs | 11.31 | 11.31 |
| 413324 | V00571 | Hs.75294 | corticotropin releasing hormone | 9.15 | 9.15 |
| 421110 | AJ250717 | Hs.1355 | cathepsin E | 9.07 | 9.07 |
| 417308 | H60720 | Hs.81892 | KIAA0101 gene product | 8.50 | 8.50 |
| 418406 | X73501 | Hs.84905 | cytokeratin 20 | 8.10 | 8.10 |
| 446619 | AU076643 | Hs.313 | secreted phosphoprotein 1 (osteopontin, | 7.98 | 7.98 |
| 433001 | AF217513 | Hs.279905 | clone HQ0310 PRO0310p1 | 7.67 | 7.67 |
| 408243 | Y00787 | Hs.624 | interleukin 8 | 7.56 | 7.56 |
| 416065 | BE267931 | Hs.78996 | proliferating cell nuclear antigen | 7.17 | 5.17 |
| 425397 | J04088 | Hs.156346 | topoisomerase (DNA) II alpha (170 kD) | 7.17 | 8.24 |
| 414183 | AW957446 | Hs.301711 | ESTs | 7.14 | 4.62 |
| 418007 | M13509 | Hs.83169 | matrix metalloproteinase 1 (interstitial | 7.12 | 7.12 |
| 426125 | X87241 | Hs.166994 | FAT tumor suppressor (Drosophila) homolo | 6.90 | 3.38 |
| 427337 | Z46223 | Hs.176663 | Fc fragment of IgG, low affinity IIIb, r | 6.85 | 4.98 |
| 441633 | AW958544 | Hs.112242 | normal mucosa of esophagus specific 1 | 6.42 | 6.42 |
| 438091 | AW373062 | | nuclear receptor subfamily 1, group l, m | 6.32 | 6.32 |
| 413063 | AL035737 | Hs.75184 | chitinase 3-like 1 (cartilage glycoprote | 6.09 | 3.67 |
| 414219 | W20010 | Hs.75823 | ALL1-fused gene from chromosome 1q | 5.93 | 4.68 |
| 405033 | | | C1002652*: gi\|544327\|sp\|Q04799\|FMO5_RABIT | 5.84 | 5.84 |
| 413132 | NM_006823 | Hs.75209 | protein kinase (cAMP-dependent, catalyti | 5.79 | 5.68 |
| 428336 | AA503115 | Hs.183752 | microseminoprotein, beta- | 5.78 | 4.57 |
| 449230 | BE613348 | Hs.211579 | melanoma cell adhesion molecule | 5.69 | 5.82 |
| 423673 | BE003054 | Hs.1695 | matrix metalloproteinase 12 (macrophage | 5.69 | 7.30 |
| 415511 | AI732617 | Hs.182362 | ESTs | 5.65 | 5.65 |
| 426028 | NM_001110 | Hs.172028 | a disintegrin and metalloproteinase doma | 5.60 | 5.60 |
| 421948 | L42583 | Hs.334309 | keratin 6A | 5.59 | 14.20 |
| 428651 | AF196478 | Hs.188401 | annexin A10 | 5.55 | 5.55 |
| 424008 | R02740 | Hs.137555 | putative chemokine receptor; GTP-binding | 5.38 | 5.59 |
| 406687 | M31126 | | matrix metalloproteinase 11 (stromelysin | 5.36 | 5.34 |
| 439453 | BE264974 | Hs.6566 | thyroid hormone receptor interactor 13 | 5.35 | 5.35 |
| 408246 | N55669 | Hs.333823 | mitochondrial ribosomal protein L13 | 5.20 | 3.50 |
| 427678 | BE267756 | Hs.180312 | mitochondrial ribosomal protein S16 | 5.13 | 4.10 |
| 426158 | NM_001982 | Hs.199067 | v-erb-b2 avian erythroblastic leukemia v | 4.97 | 3.71 |
| 442315 | AA173992 | Hs.7956 | ESTs, Moderately similar to ZN91_HUMAN Z | 4.90 | 4.90 |
| 418322 | AA284166 | Hs.84113 | cyclin-dependent kinase inhibitor 3 (CDK | 4.90 | 4.77 |
| 417720 | AA205625 | Hs.208067 | ESTs | 4.84 | 7.34 |
| 423979 | AF229181 | Hs.136644 | CS box-containing WD protein | 4.81 | 4.81 |
| 420981 | L40904 | Hs.100724 | peroxisome proliferative activated recep | 4.81 | 4.43 |
| 433470 | AW960564 | | transmembrane 4 superfamily member 1 | 4.72 | 4.72 |
| 429138 | AB020657 | Hs.197298 | NS1-binding protein | 4.71 | 4.71 |
| 408063 | BE086548 | Hs.42346 | calcineurin-binding protein calsarcin-1 | 4.71 | 4.71 |
| 452714 | AW770994 | Hs.30340 | hypothetical protein KIAA1165 | 4.69 | 4.69 |
| 442432 | BE093589 | Hs.38178 | hypothetical protein FLJ23468 | 4.68 | 4.68 |
| 424834 | AK001432 | Hs.153408 | Homo sapiens cDNA FLJ10570 fis, clone NT | 4.65 | 4.65 |
| 446921 | AB012113 | Hs.16530 | small inducible cytokine subfamily A (Cy | 4.64 | 4.64 |
| 427490 | Z95152 | Hs.178695 | mitogen-activated protein kinase 13 | 4.63 | 5.12 |
| 412490 | AW803564 | Hs.288850 | Homo sapiens cDNA: FLJ22528 fis, clone H | 4.61 | 4.61 |
| 418030 | BE207573 | Hs.83321 | neuromedin B | 4.60 | 4.60 |
| 401192 | | | Target Exon | 4.60 | 4.29 |
| 426761 | AI015709 | Hs.172089 | Homo sapiens mRNA; cDNA DKFZp586I2022 (f | 4.59 | 3.51 |
| 452747 | BE153855 | Hs.61460 | Ig superfamily receptor LNIR | 4.58 | 5.33 |
| 449618 | AI076459 | Hs.15978 | KIAA1272 protein | 4.58 | 4.58 |
| 423725 | AJ403108 | Hs.132127 | hypothetical protein LOC57822 | 4.55 | 4.55 |
| 415701 | NM_003878 | Hs.78619 | gamma-glutamyl hydrolase (conjugase, fol | 4.52 | 4.70 |
| 446742 | AA232119 | Hs.16085 | putative G-protein coupled receptor | 4.49 | 4.11 |
| 419433 | AA814807 | Hs.7395 | hypothetical protein FLJ23182 | 4.48 | 4.48 |
| 412326 | R07566 | Hs.73817 | small inducible cytokine A3 (homologous | 4.47 | 4.47 |
| 427528 | AU077143 | Hs.179565 | minichromosome maintenance deficient (S. | 4.45 | 4.45 |
| 444371 | BE540274 | Hs.239 | forkhead box M1 | 4.44 | 3.87 |
| 444006 | BE395085 | Hs.10086 | type I transmembrane protein Fn14 | 4.43 | 3.63 |
| 424308 | AW975531 | Hs.154443 | minichromosome maintenance deficient (S. | 4.43 | 4.43 |
| 401093 | | | C12000586*: gi\|6330167\|dbj\|BAA86477.1\| (A | 4.40 | 4.40 |
| 447644 | AW861622 | Hs.108646 | Homo sapiens cDNA FLJ14934 fis, clone PL | 4.39 | 4.39 |
| 417933 | X02308 | Hs.82962 | thymidylate synthetase | 4.38 | 4.35 |
| 409461 | AA382169 | Hs.54483 | N-myc (and STAT) interactor | 4.36 | 3.68 |
| 401451 | | | NM_004496*: Homo sapiens hepatocyte nucle | 4.35 | 4.35 |
| 450746 | D82673 | Hs.278589 | general transcription factor II, i | 4.35 | 3.36 |
| 414683 | S78296 | Hs.76888 | hypothetical protein MGC12702 | 4.34 | 4.74 |
| 434203 | BE262677 | Hs.283558 | hypothetical protein PRO1855 | 4.31 | 4.31 |
| 417615 | BE548641 | Hs.82314 | hypoxanthine phosphoribosyltransferase 1 | 4.30 | 4.30 |
| 416815 | U41514 | Hs.80120 | UDP-N-acetyl-alpha-D-galactosamine: polyp | 4.30 | 4.30 |
| 440086 | NM_005402 | Hs.288757 | v-ral simian leukemia viral oncogene hom | 4.29 | 4.29 |
| 417715 | AW969587 | Hs.86366 | ESTs | 4.27 | 7.45 |
| 409757 | NM_001898 | Hs.123114 | cystatin SN | 4.24 | 3.39 |
| 412140 | AA219691 | Hs.73625 | RAB6 interacting, kinesin-like (rabkines | 4.24 | 4.24 |
| 432842 | AW674093 | Hs.334822 | hypothetical protein MGC4485 | 4.21 | 4.12 |
| 446847 | T51454 | Hs.82845 | Homo sapiens cDNA: FLJ21930 fis, clone H | 4.20 | 4.20 |
| 436856 | AI469355 | Hs.127310 | ESTs | 4.19 | 4.19 |
| 428450 | NM_014791 | Hs.184339 | KIAA0175 gene product | 4.16 | 4.90 |
| 425234 | AW152225 | Hs.165909 | ESTs, Weakly similar to I38022 hypotheti | 4.14 | 4.14 |
| 409231 | AA446644 | Hs.692 | GA733-2 antigen; epithelial glycoprotein | 4.14 | 5.99 |
| 426283 | NM_003937 | Hs.169139 | kynureninase (L-kynurenine hydrolase) | 4.12 | 4.12 |
| 446849 | AU076617 | Hs.16251 | cleavage and polyadenylation specific fa | 4.12 | 3.43 |
| 400843 | | | NM_003105*: Homo sapiens sortilin-related | 4.11 | 5.51 |
| 449722 | BE280074 | Hs.23960 | cyclin B1 | 4.09 | 4.09 |
| 405506 | | | Target Exon | 4.09 | 3.75 |
| 420344 | BE463721 | Hs.97101 | putative G protein-coupled receptor | 4.07 | 4.07 |
| 426997 | BE620738 | Hs.173125 | peptidylprolyl isomerase F (cyclophilin | 4.05 | 4.80 |
| 456525 | AW468397 | Hs.100000 | S100 calcium-binding protein A8 (calgran | 4.03 | 7.64 |
| 437150 | R51407 | Hs.77910 | 3-hydroxy-3-methylglutaryl-Coenzyme A sy | 4.02 | 4.02 |
| 413794 | AF234532 | Hs.61638 | myosin X | 4.02 | 4.02 |
| 422511 | AU076442 | Hs.117938 | collagen, type XVII, alpha 1 | 4.02 | 4.72 |
| 414020 | NM_002984 | Hs.75703 | small inducible cytokine A4 (homologous | 4.01 | 3.99 |
| 416391 | AI878927 | Hs.79284 | mesoderm specific transcript (mouse) hom | 4.01 | 4.01 |
| 422809 | AK001379 | Hs.121028 | hypothetical protein FLJ10549 | 4.00 | 4.00 |
| 400277 | | | Eos Control | 4.00 | 3.47 |
| 415791 | H09366 | Hs.78853 | uracil-DNA glycosylase | 3.99 | 3.37 |
| 412610 | X90908 | Hs.74126 | fatty acid binding protein 6, ileal (gas | 3.98 | 4.77 |
| 427557 | NM_002659 | Hs.179657 | plasminogen activator, urokinase recepto | 3.98 | 3.95 |
| 413753 | U17760 | Hs.75517 | laminin, beta 3 (nicein (125 kD), kalinin | 3.96 | 6.95 |
| 420859 | AW468397 | Hs.100000 | S100 calcium-binding protein A8 (calgran | 3.92 | 5.04 |
| 400409 | AF153341 | | Homo sapiens winged helix/forkhead trans | 3.91 | 3.88 |
| 408988 | AL119844 | Hs.49476 | Homo sapiens clone TUA8 Cri-du-chat regi | 3.90 | 3.90 |
| 411678 | AI907114 | Hs.71465 | squalene epoxidase | 3.89 | 3.89 |
| 429113 | D28235 | Hs.196384 | prostaglandin-endoperoxide synthase 2 (p | 3.87 | 3.87 |
| 428428 | AL037544 | Hs.184298 | cyclin-dependent kinase 7 (homolog of Xe | 3.87 | 3.87 |
| 442932 | AA457211 | Hs.8858 | bromodomain adjacent to zinc finger doma | 3.85 | 4.50 |
| 429083 | Y09397 | Hs.227817 | BCL2-related protein A1 | 3.85 | 3.85 |
| 439963 | AW247529 | Hs.6793 | platelet-activating factor acetylhydrola | 3.82 | 3.77 |
| 441362 | BE614410 | Hs.23044 | RAD51 (S. cerevisiae) homolog (E coli Re | 3.82 | 3.82 |
| 430589 | AJ002744 | Hs.246315 | UDP-N-acetyl-alpha-D-galactosamine: polyp | 3.81 | 3.81 |
| 417771 | AA804698 | Hs.82547 | retinoic acid receptor responder (tazaro | 3.81 | 3.62 |
| 430259 | BE550182 | Hs.127826 | RalGEF-like protein 3, mouse homolog | 3.80 | 3.80 |
| 447973 | AB011169 | Hs.20141 | similar to S. cerevisiae SSM4 | 3.77 | 3.77 |
| 404875 | | | NM_022819*: Homo sapiens phospholipase A2 | 3.77 | 3.46 |
| 411299 | BE409857 | Hs.69499 | hypothetical protein | 3.76 | 3.76 |
| 418827 | BE327311 | Hs.47166 | HT021 | 3.76 | 3.76 |
| 446839 | BE091926 | Hs.16244 | mitotic spindle coiled-coil related prot | 3.75 | 3.75 |
| 407137 | T97307 | | gb: ye53h05.s1 Soares fetal liver spleen | 3.73 | 3.73 |
| 433376 | AI249361 | Hs.74122 | caspase 4, apoptosis-related cysteine pr | 3.71 | 3.71 |
| 400294 | N95796 | Hs.278695 | Homo sapiens prostein mRNA, complete cds | 3.70 | 3.45 |
| 409518 | BE384836 | Hs.3454 | KIAA1821 protein | 3.69 | 3.69 |
| 430024 | AI808780 | Hs.227730 | integrin, alpha 6 | 3.69 | 3.69 |
| 426088 | AF038007 | Hs.166196 | ATPase, Class I, type 8B, member 1 | 3.68 | 3.68 |
| 418478 | U38945 | Hs.1174 | cyclin-dependent kinase inhibitor 2A (me | 3.68 | 3.66 |
| 414761 | AU077228 | Hs.77256 | enhancer of zeste (Drosophila) homolog 2 | 3.67 | 3.67 |
| 413670 | AB000115 | Hs.75470 | hypothetical protein, expressed in osteo | 3.67 | 3.41 |
| 424840 | D79987 | Hs.153479 | extra spindle poles, S. cerevisiae, homo | 3.67 | 3.88 |
| 434263 | N34895 | Hs.44648 | ESTs | 3.65 | 3.65 |
| 438280 | AW015534 | Hs.217493 | annexin A2 | 3.63 | 3.36 |
| 443426 | AF098158 | Hs.9329 | chromosome 20 open reading frame 1 | 3.63 | 3.68 |
| 408989 | AW361666 | Hs.49500 | KIAA0746 protein | 3.61 | 3.66 |
| 416640 | BE262478 | Hs.79404 | neuron-specific protein | 3.60 | 4.22 |
| 416926 | H03109 | Hs.108920 | HT018 protein | 3.59 | 3.59 |
| 414368 | W70171 | Hs.75939 | uridine monophosphate kinase | 3.59 | 3.53 |
| 402727 | | | NM_025065: Homo sapiens hypothetical prot | 3.58 | 3.58 |
| 419381 | AB023420 | Hs.90093 | heat shock 70 kD protein 4 | 3.56 | 3.77 |
| 416114 | AI695549 | Hs.183868 | glucuronidase, beta | 3.55 | 3.55 |
| 424941 | AA128376 | Hs.153884 | ATP binding protein associated with cell | 3.55 | 3.55 |
| 431958 | X63629 | Hs.2877 | cadherin 3, type 1, P-cadherin (placenta | 3.54 | 4.63 |
| 429238 | NM_002849 | Hs.198288 | protein tyrosine phosphatase, receptor t | 3.53 | 3.92 |
| 420159 | AI572490 | Hs.99785 | Homo sapiens cDNA: FLJ21245 fis, clone C | 3.51 | 5.77 |
| 400289 | X07820 | Hs.2258 | matrix metalloproteinase 10 (stromelysin | 3.50 | 3.50 |
| 418203 | X54942 | Hs.83758 | CDC28 protein kinase 2 | 3.50 | 3.50 |
| 415220 | AA431880 | Hs.181174 | ESTs, Weakly similar to T19201 hypotheti | 3.50 | 3.50 |
| 428371 | AB012193 | Hs.183874 | cullin 4A | 3.46 | 3.46 |
| 418663 | AK001100 | Hs.41690 | desmocollin 3 | 3.45 | 4.74 |
| 404977 | | | Insulin-like growth factor 2 (somatomedi | 3.45 | 3.89 |
| 422663 | AW500087 | Hs.119014 | zinc finger protein 175 | 3.44 | 3.44 |
| 434061 | AW024973 | Hs.283675 | NPD009 protein | 3.41 | 5.64 |
| 418113 | AI272141 | Hs.83484 | SRY (sex determining region Y)-box 4 | 3.41 | 4.32 |
| 431689 | AA305688 | Hs.267695 | UDP-Gal: betaGlcNAc beta 1,3-galactosyltr | 3.40 | 3.40 |
| 411943 | BE502436 | Hs.7962 | ESTs, Weakly similar to S44608 C02F5.6 p | 3.39 | 4.27 |
| 420005 | AW271106 | Hs.133294 | ESTs | 3.38 | 3.40 |
| 453450 | AW797627 | Hs.347459 | ADP-ribosylation factor 6 | 3.38 | 3.87 |
| 410315 | AI638871 | Hs.17625 | Homo sapiens cDNA: FLJ22524 fis, clone H | 3.36 | 3.36 |
| 428839 | AI767756 | Hs.82302 | Homo sapiens cDNA FLJ14814 fis, clone NT | 3.35 | 3.35 |
| 437469 | AW753112 | Hs.15514 | hypothetical protein MGC3260 | 3.35 | 3.35 |
| 407151 | H25836 | Hs.301527 | ESTs, Moderately similar to unknown [H. s | 3.34 | 3.34 |
| 428157 | AI738719 | Hs.198427 | hexokinase 2 | 3.33 | 3.73 |
| 450293 | N36754 | Hs.171118 | hypothetical protein FLJ00026 | 3.33 | 3.33 |
| 400750 | | | Target Exon | 3.33 | 3.33 |
| 450139 | AK001838 | Hs.296323 | serum/glucocorticoid regulated kinase | 3.33 | 3.33 |
| 412636 | NM_004415 | | desmoplakin (DPI, DPII) | 3.30 | 4.81 |
| 447578 | AA912347 | Hs.136585 | ESTs, Weakly similar to JC5314 CDC28/cdc | 3.27 | 3.38 |
| 430315 | NM_004293 | Hs.239147 | guanine deaminase | 3.26 | 4.30 |
| 421594 | R45689 | Hs.21889 | Homo sapiens cDNA FLJ12978 fis, clone NT | 3.26 | 3.41 |
| 443030 | R68048 | Hs.9238 | hypothetical protein FLJ23516 | 3.19 | 3.34 |
| 436911 | AA142984 | Hs.5344 | adaptor-related protein complex 1, gamma | 3.17 | 3.40 |
| 440006 | AK000517 | Hs.6844 | hypothetical protein FLJ20510 | 3.06 | 3.52 |
| 443171 | BE281128 | Hs.9030 | TONDU | 3.05 | 3.83 |
| 429343 | AK000785 | Hs.199480 | Homo sapiens, Similar to epsin 3, clone | 3.01 | 3.53 |
| 408380 | AF123050 | Hs.44532 | diubiquitin | 2.99 | 4.11 |
| 421508 | NM_004833 | Hs.105115 | absent in melanoma 2 | 2.99 | 3.67 |
| 439750 | AL359053 | Hs.57664 | Homo sapiens mRNA full length insert cDN | 2.97 | 4.55 |
| 452046 | AB018345 | Hs.27657 | KIAA0802 protein | 2.95 | 3.39 |
| 451940 | AI735759 | Hs.52620 | integrin, beta 8 | 2.93 | 3.58 |
| 407722 | BE252241 | Hs.38041 | pyridoxal (pyridoxine, vitamin B6) kinas | 2.90 | 3.68 |
| 422282 | AF019225 | Hs.114309 | apolipoprotein L | 2.89 | 3.57 |
| 402230 | | | Target Exon | 2.88 | 5.36 |
| 406685 | M18728 | | gb: Human nonspecific crossreacting antig | 2.80 | 5.80 |
| 417880 | BE241595 | Hs.82848 | selectin L (lymphocyte adhesion molecule | 2.79 | 3.89 |
| 447957 | NM_014821 | Hs.20126 | KIAA0317 gene product | 2.75 | 3.45 |
| 418004 | U37519 | Hs.87539 | aldehyde dehydrogenase 3 family, member | 2.75 | 3.46 |
| 417275 | X63578 | Hs.295449 | parvalbumin | 2.73 | 3.54 |
| 431211 | M86849 | Hs.323733 | gap junction protein, beta 2, 26 kD (conn | 2.72 | 8.39 |
| 401781 | | | Target Exon | 2.62 | 4.15 |
| 407242 | M18728 | | gb: Human nonspecific crossreacting antig | 2.54 | 5.96 |
| 428423 | AU076517 | Hs.184276 | solute carrier family 9 (sodium/hydrogen | 2.52 | 4.27 |
| 430200 | BE613337 | Hs.234896 | geminin | 2.52 | 4.19 |
| 451035 | AU076785 | Hs.430 | plastin 1 (I isoform) | 2.51 | 4.15 |
| 443162 | T49951 | Hs.9029 | DKFZP434G032 protein | 2.48 | 3.66 |
| 441495 | AW294603 | Hs.127039 | ESTs | 2.45 | 3.60 |
| 449246 | AW411209 | Hs.23363 | hypothetical protein FLJ10983 | 2.45 | 3.52 |
| 401780 | | | NM_005557*: Homo sapiens keratin 16 (foca | 2.22 | 4.49 |
| 417079 | U65590 | Hs.81134 | interleukin 1 receptor antagonist | 2.20 | 3.55 |
| 422168 | AA586894 | Hs.112408 | S100 calcium-binding protein A7 (psorias | 2.15 | 6.08 |
| 439394 | AA149250 | Hs.56105 | ESTs | 2.05 | 3.95 |
| 427315 | AA179949 | Hs.175563 | Home sapiens mRNA; cDNA DKFZp564N0763 (f | 1.79 | 3.88 |

Pkey: Unique Eos probeset identifier number
ExAccn: Exemplar Accession number, Genbank accession number
UnigeneID: Unigene number
Unigene Title: Unigene gene title
R1 90th percentile of bladder tumor Als divided by the 90th percentile of normal urothelium biopsy Ais
R2 90th percentile of bladder tumor Als divided by the 90th percentile of normal urothelium biopsy and normal bladder Als

TABLE 6B

| Pkey | CAT Number | Accession |
|---|---|---|
| 412636 | 13165_1 | NM_004415 AL031058 M77830 BE149760 AW752599 AW848723 AW376697 AW376817 |
| | | AW376699 AW848371 AW376782 AW848789 AW361413 AW849074 AW997139 AW799304 |
| | | AW799309 BE077020 BE077017 BE185187 AW997196 BE156621 BE179915 BE006561 |
| | | BE143155 AW890985 BE002107 AW103521 AA857316 AW383133 BE011378 AW170253 |
| | | BE185750 AW886475 BE160433 J05211 BE082576 BE082584 BE004047 AW607238 |
| | | AW377700 AW377699 BE082526 BE082505 BE082507 BE082514 AW178000 AW177933 |
| | | AI905935 AW747877 AW748114 BE148516 AW265328 AW847678 AW847688 AW365151 |
| | | AW365148 AW365153 AW365156 AW365175 AW365157 AW365154 AW068840 BE005272 |
| | | AW365145 BE001925 BE182166 BE144243 BE001923 AI951766 AI434518 BE184920 |
| | | BE184933 AI284090 BE184941 AW804674 BE184924 C04715 W39488 AW995615 |
| | | BE184948 BE159646 AW606653 AA099891 AA131128 AA337270 AA340777 AW384371 |
| | | AA852212 R58704 AW366566 AW364859 AA025851 AA025852 AA455100 AA719958 |
| | | AW352220 AW996245 BE165351 BE073467 AA377127 AW890264 AW609750 AW391912 |
| | | AW849690 T87267 AW853812 AA852213 W74149 BE009090 AA056401 H91011 |
| | | AW368529 AW390272 C18467 AW674920 N57176 AA026480 AW576767 H93284 |
| | | AA026863 AW177787 AA026654 AW177786 BE092134 BE092137 BE092136 AW177784 |
| | | AI022862 BE091653 AW376811 AW848592 AA040018 BE185331 BE182164 AA368564 |
| | | AW951576 T29918 AA131077 W95048 W25458 AW205789 H90899 N29754 |
| | | W32490 R20904 BE167181 BE167165 N84767 H27408 H30146 AI190590 |
| | | C03378 AI554403 AI205263 AA128470 AI392926 AF139065 AW370813 AW370827 |
| | | AW798417 AW798780 AW798883 AW798569 R33557 AA149190 C03029 AW177783 |
| | | AA088866 AW370829 AA247685 BE002273 AI760816 AI439101 AW879451 AI700963 |
| | | AA451923 AI340326 AI590975 T48793 AI568096 AI142882 AA039975 AI470146 |
| | | AA946936 BE067737 BE067786 W19287 AA644381 AA702424 AI417612 AI306554 |
| | | AI686869 AI568892 AW190555 AI571075 AI220573 AA056527 AI471874 AI304772 |
| | | AW517828 AI915596 AI627383 AI270345 AW021347 AW166807 AW105614 AI346078 |
| | | AA552300 W95070 AI494069 AI911702 AA149191 AA026864 AI830049 AI887258 |
| | | AW780435 AI910434 AI819984 AI858282 AI078449 AI025932 AI860584 AI635878 |
| | | AA026047 AA703232 D12062 AW192085 AA658154 AW514597 AW591892 T87181 |
| | | AA782066 AW243815 AW150038 AW268383 AW004633 AI927207 AA782109 AW473233 |
| | | AI804485 AW169216 AI572669 AA602182 AW015480 AW771865 AI270027 AA961816 |
| | | AA283207 AI076962 AI498487 AI348053 AI783914 H44405 AW799118 AA128330 |
| | | AA515500 AA918281 W02156 AI905927 AA022701 W38382 R20795 T77861 |
| | | AW860878 |
| 433470 | 3672_1 | AW960564 AA092457 T55890 D56120 T92525 AI815987 BE182608 BE182595 |
| | | AW080238 M90657 AA347236 AW961686 AW176446 AA304671 AW583735 T61714 |
| | | AA316968 AI446615 AA343532 AA083489 AA488005 W52095 W39480 N57402 |
| | | D82638 W25540 W52847 D82729 D58990 BE619182 AA315188 AA308636 |
| | | AA112474 W76162 AA088544 H52265 AA301631 H80982 AA113786 BE620997 |
| | | AW651691 AA343799 BE613669 BE547180 BE546656 F11933 AA376800 AW239185 |
| | | AA376086 BE544387 BE619041 AA452515 AA001806 AA190873 AA180483 AA159546 |
| | | F00242 AI940609 AI940602 AI189753 T97663 T66110 AW062896 AW062910 |
| | | AW062902 AI051622 AI828930 AA102452 AI685095 AI819390 AA557597 AA383220 |
| | | AI804422 AI633575 AW338147 AW603423 AW606800 AW750567 AW510672 AI250777 |
| | | AA083510 AW629109 AW513200 AA921353 AI677934 AI148698 AI955858 AA173825 |
| | | AA453027 AI027865 AW375542 AA454099 AA733014 AI591384 R79300 R80023 |
| | | AA843108 AA626058 AA844898 AW375550 AA889018 AI474275 AW205937 AI052270 |
| | | AW388117 AW388111 AA699452 AI242230 N47476 H38178 AA366621 AA113196 |
| | | AA130023 H39740 T61629 AI885973 AW083671 AA179730 AA305757 AI285455 |
| | | N83956 AA216013 AA336155 AW999959 T97525 AA345349 T91762 AA771981 |
| | | AI285092 AI591386 BE392486 BE385852 AA682601 AI682884 AA345840 T85477 |
| | | AA292949 AA932079 AA098791 D82607 T48574 AW752038 C06300 |
| 438091 | 44964_1 | AW373062 T55662 AI299190 BE174210 AW579001 H01811 W40186 R67100 |
| | | AI923886 AW952164 AA628440 AW898607 AW898616 AA709126 AW898628 AW898544 |
| | | AA947932 AW898625 AW898622 AI276125 AI185720 AW510698 AA987230 T52522 |
| | | BE467708 AW243400 AW043642 AI288245 AI186932 D52654 D55017 D52715 |
| | | D52477 D53933 D54679 AI298739 AI146984 AI922204 N98343 BE174213 |
| | | AA845571 AI813854 AI214518 AI635262 AI139455 AI707807 AI698085 AW884528 |
| | | AI024768 AI004723 AW087420 AI565133 N94964 AI268939 AW513280 AI061126 |
| | | AI435818 AI859106 AI360506 AI024767 AA513019 AA757598 X56196 AA902959 |
| | | AI334784 AI860794 AA010207 AW890091 AW513771 AI951391 AI337671 T52499 |
| | | AA890205 AI640908 H75966 AA463487 AA358688 AI961767 AI866295 AA780994 |
| | | AI985913 BE174196 AA029094 AW592159 T55581 N79072 AI611201 AA910812 |
| | | AI220713 AW149306 AI758412 AA045713 R79750 N76096 |

Pkey: Unique Eos probeset identifier number
CAT number: Gene cluster number
Accession: Genbank accession numbers

TABLE 6C

| Pkey | Ref | Strand | Nt_position |
|---|---|---|---|
| 400750 | 8119067 | Plus | 198991-199168, 199316-199548 |
| 400843 | 9188605 | Plus | 5863-5970, 7653-7784, 8892-9023, |
| | | | 9673-9807, 10634-10789, |
| | | | 15254-15403, 23827-23958 |
| 401093 | 8516137 | Minus | 22335-23166 |
| 401192 | 9719502 | Minus | 69559-70101 |
| 401451 | 6634068 | Minus | 119926-121272 |
| 401780 | 7249190 | Minus | 28397-28617, 28920-29045, |
| | | | 29135-29296, 29411-29567, |
| | | | 29705-29787, 30224-30573 |
| 401781 | 7249190 | Minus | 83215-83435, 83531-83656, |
| | | | 83740-83901, 84237-84393, |
| | | | 84955-85037, 86290-86814 |
| 402230 | 9966312 | Minus | 29782-29932 |
| 402727 | 9211324 | Plus | 54596-54777 |
| 404875 | 9801324 | Plus | 96588-96732, 97722-97831 |
| 404977 | 3738341 | Minus | 43081-43229 |
| 405033 | 7107731 | Minus | 142358-142546 |
| 405506 | 6466489 | Plus | 80014-80401, 80593-81125 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 7A
Genes downregulated in bladder cancer

| Pkey | ExAccn | UnigeneID | Unigene Title | R1 | R2 |
|---|---|---|---|---|---|
| 403010 | | | C21000152: gi\|6226483\|sp\|Q52118\|YMO3_ERWS | 4.86 | 2.49 |
| 426796 | S78234 | Hs.172405 | cell division cycle 27 | 4.28 | 2.48 |
| 416225 | AA577730 | Hs.188684 | ESTs, Weakly similar to PC4259 ferritin | 4.04 | 2.07 |
| 459006 | AW298631 | Hs.27721 | Wolf-Hirschhorn syndrome candidate 1-lik | 3.82 | 2.66 |
| 404917 | | | Target Exon | 3.78 | 2.00 |
| 426488 | X03350 | Hs.4 | alcohol dehydrogenase 1B (class I), beta | 2.64 | 1.79 |
| 419543 | AA244170 | | gb: nc05h02.s1 NCI_CGAP_Pr1 Homo sapiens | 2.63 | 3.42 |
| 453180 | N46243 | Hs.110373 | ESTs, Highly similar to T42626 secreted | 2.32 | 3.24 |
| 428957 | NM_003881 | Hs.194679 | WNT1 inducible signaling pathway protein | 2.20 | 3.80 |
| 451529 | AI917901 | Hs.208641 | ESTs | 2.18 | 3.69 |
| 417076 | AW973454 | Hs.238442 | ESTs, Moderately similar to ALU7_HUMAN A | 2.03 | 3.03 |
| 425438 | T62216 | Hs.270840 | ESTs | 2.00 | 5.17 |
| 450515 | AW304226 | | biphenyl hydrolase-like (serine hydrolas | 1.89 | 2.46 |
| 432873 | AW837268 | Hs.279639 | Homo sapiens mRNA; cDNA DKFZp586M2022 (f | 1.70 | 2.79 |
| 452123 | AI267615 | Hs.38022 | ESTs | 1.69 | 2.46 |
| 424378 | W28020 | Hs.167988 | neural cell adhesion molecule 1 | 1.65 | 4.67 |
| 437601 | AA761546 | Hs.248844 | ESTs, Weakly similar to ALU1_HUMAN ALU S | 1.50 | 3.34 |
| 402096 | | | ENSP00000217725*: Laminin alpha-1 chain p | 1.48 | 3.02 |
| 439563 | AI018768 | Hs.12482 | glyceronephosphate O-acyltransferase | 1.47 | 3.22 |
| 412810 | M21574 | Hs.74615 | platelet-derived growth factor receptor, | 1.46 | 2.30 |
| 458651 | AW612481 | Hs.104105 | ESTs | 1.39 | 2.89 |
| 414033 | AL079707 | Hs.207443 | hypothetical protein MGC10848 | 1.36 | 2.80 |
| 433572 | AL046859 | Hs.3407 | protein kinase (cAMP-dependent, catalyti | 1.35 | 3.49 |
| 413305 | NM_000426 | Hs.323511 | Homo sapiens cDNA: FLJ23176 fis, clone L | 1.34 | 2.93 |
| 420412 | AW976674 | Hs.125103 | ESTs | 1.32 | 5.13 |
| 421406 | AF179897 | Hs.104105 | Meis (mouse) homolog 2 | 1.31 | 4.07 |
| 417446 | AL118671 | Hs.82163 | monoamine oxidase B | 1.27 | 2.86 |
| 452886 | AI478250 | Hs.13751 | ESTs | 1.26 | 1.95 |
| 446808 | AA703226 | Hs.16193 | Homo sapiens mRNA; cDNA DKFZp586B211 (fr | 1.25 | 3.44 |
| 443105 | X96753 | Hs.9004 | chondroitin sulfate proteoglycan 4 (mela | 1.24 | 2.07 |
| 421348 | M94048 | Hs.103724 | peripheral myelin protein 22 | 1.24 | 2.63 |
| 433070 | N75346 | Hs.306121 | CDC20 (cell division cycle 20, S. cerevi | 1.23 | 2.80 |
| 420059 | AF161486 | Hs.94769 | RAB23, member RAS oncogene family | 1.22 | 3.43 |
| 408491 | AI088063 | Hs.7882 | ESTs | 1.20 | 6.01 |
| 447384 | AI377221 | Hs.40528 | ESTs | 1.00 | 7.92 |
| 421998 | R74441 | Hs.117176 | poly(A)-binding protein, nuclear 1 | 1.00 | 7.38 |
| 409619 | AK001015 | Hs.55220 | BCL2-associated athanogene 2 | 1.00 | 6.40 |
| 444795 | AI193356 | Hs.160316 | ESTs | 1.00 | 5.53 |
| 408495 | W68796 | Hs.237731 | ESTs | 1.00 | 5.05 |
| 417124 | BE122762 | Hs.25338 | ESTs | 1.00 | 4.73 |
| 443998 | AI620661 | Hs.296276 | ESTs | 1.00 | 4.39 |
| 406303 | | | C16000922: gi\|7499103\|pir∥T20903 hypothe | 1.00 | 4.37 |
| 422994 | AW891802 | Hs.296276 | ESTs | 1.00 | 4.37 |
| 422195 | AB007903 | Hs.113082 | KIAA0443 gene product | 1.00 | 4.35 |
| 452877 | AI250789 | Hs.32478 | ESTs | 1.00 | 3.90 |
| 452487 | AW207659 | Hs.6630 | Homo sapiens cDNA FLJ13329 fis, clone OV | 1.00 | 3.90 |
| 417159 | R01761 | | gb: ye81f10.s1 Soares fetal liver spleen | 1.00 | 3.82 |
| 445607 | AA488107 | Hs.30156 | ESTs, Weakly similar to unnamed protein | 1.00 | 3.62 |
| 406274 | | | Target Exon | 1.00 | 3.59 |
| 410611 | AW954134 | Hs.20924 | KIAA1628 protein | 1.00 | 3.06 |
| 426495 | NM_001151 | Hs.2043 | solute carrier family 25 (mitochondrial | 1.00 | 2.89 |
| 422292 | AI815733 | Hs.114360 | transforming growth factor beta-stimulat | 1.00 | 2.61 |
| 413040 | AA193338 | Hs.12321 | sodium calcium exchanger | 1.00 | 2.51 |
| 429623 | NM_005308 | Hs.211569 | G protein-coupled receptor kinase 5 | 1.00 | 2.05 |
| 456607 | AI660190 | Hs.106070 | cyclin-dependent kinase inhibitor 1C (p5 | 1.00 | 2.01 |
| 429143 | AA333327 | Hs.197335 | plasma glutamate carboxypeptidase | 0.97 | 2.45 |
| 400288 | X06256 | Hs.149609 | integrin, alpha 5 (fibronectin receptor, | 0.90 | 2.47 |
| 442498 | U54617 | Hs.8364 | Homo sapiens pyruvate dehydrogenase kina | 0.88 | 5.08 |
| 414449 | AA557660 | Hs.76152 | decorin | 0.88 | 3.13 |
| 412014 | AI620650 | Hs.43761 | ESTs, Weakly similar to A46010 X-linked | 0.78 | 1.88 |
| 425100 | AF051850 | Hs.154567 | supervillin | 0.70 | 3.90 |
| 432094 | AI658580 | Hs.61426 | Homo sapiens mesenchymal stem cell prote | 0.68 | 3.41 |
| 427818 | AW511222 | Hs.193765 | ESTs | 0.63 | 3.75 |

Pkey: Unique Eos probeset identifier number
ExAccn: Exemplar Accession number, Genbank accession number
UnigeneID: Unigene number
Unigene Title: Unigene gene title
R1 90th percentile of normal urothelium biopsy Als divided by 75th percentile of bladder tumor Ais
R2 90th percentile of normal urothelium biopsy and normal bladder Als divided by the 90th percentile bladder tumor Als

TABLE 7B

| Pkey | CAT Number | Accession |
|---|---|---|
| 417159 | 1653899_1 | R01761 R01760 N49787 |
| 419543 | 185745_1 | AA244170 AI018087 AA244355 |
| 450515 | 83710_1 | AW304226 AW008420 AA349212 H15015 AA317021 AI829484 H25661 H81744 AI906147 |
| | | AA837938 AW167766 AW603578 AW842369 BE439926 AA902417 AW235409 AA010062 AW069319 |
| | | AI280242 AW672925 H06848 H05608 R51905 R45023 AW675471 H28475 AI086597 |
| | | AI197815 AI825355 N99134 AI075956 AI470122 AA449985 AW662833 AA860423 AA913342 |
| | | T23825 AI394207 AI310319 T32467 AI589870 AI682293 AI810633 BE223045 H14620 |
| | | AA626645 AA876023 T33571 AA953982 AI138631 H15016 AI304356 AA983631 AI350990 |
| | | AI143993 AI708171 AA526961 H26247 W38485 AA847598 H81745 AW855486 BE299605 |
| | | AI079409 AI278050 AI223168 AI860904 AW025415 AI339003 AA393692 AI354302 AI492838 |
| | | N80194 AI015651 N34543 BE295397 AI085154 W24135 |

Pkey: Unique Eos probeset identifier number
CAT number: Gene cluster number
Accession: Genbank accession numbers

TABLE 7C

| Pkey | Ref | Strand | Nt_position |
|---|---|---|---|
| 402096 | 8117697 | Minus | 24993-25186 |
| 403010 | 3132346 | Plus | 78385-79052 |
| 404917 | 7341851 | Plus | 49330-49498 |
| 406274 | 7543787 | Plus | 932-1123 |
| 406303 | 8575868 | Plus | 173622-173786 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 8A
Genes predictive of bladder cancer progression

| Pkey | ExAccn | UnigeneID | Unigene Title | R1 | R2 |
|---|---|---|---|---|---|
| 413324 | V00571 | Hs.75294 | corticotropin releasing hormone | 8.30 | 4.18 |
| 437802 | AI475995 | Hs.122910 | ESTs | 7.51 | 2.50 |
| 444444 | AI149332 | Hs.14855 | ESTs | 2.58 | 1.38 |
| 445033 | AV652402 | Hs.72901 | mucin 13, epithelial transmembrane | 2.26 | 1.13 |
| 417771 | AA804698 | Hs.82547 | retinoic acid receptor responder (tazaro | 3.27 | 5.33 |
| 449618 | AI076459 | Hs.15978 | KIAA1272 protein | 2.70 | 3.33 |
| 407242 | M18728 | | gb: Human nonspecific crossreacting antig | 3.58 | 1.90 |
| 418318 | U47732 | Hs.84072 | transmembrane 4 superfamily member 3 | 3.32 | 1.00 |
| 423441 | R68649 | Hs.278359 | absent in melanoma 1 like | 2.44 | 2.40 |
| 405033 | | | C1002652*: gi\|544327\|sp\|Q04799\|FMO5_RABlT | 1.75 | 3.48 |
| 423024 | AA593731 | Hs.325823 | ESTs, Moderately similar to ALU5_HUMAN A | 3.72 | 1.28 |
| 425118 | AU076611 | Hs.154672 | methylene tetrahydrofolate dehydrogenase | 2.40 | 2.78 |
| 437928 | NM_005476 | Hs.5920 | UDP-N-acetylglucosamine-2-epimerase/N-ac | 2.20 | 1.53 |
| 446584 | U53445 | Hs.15432 | downregulated in ovarian cancer 1 | 1.38 | 2.33 |
| 436608 | AA628980 | | down syndrome critical region protein DS | 3.32 | 4.53 |
| 404440 | | | NM_021048: Homo sapiens melanoma antigen, | 2.64 | 1.00 |
| 435602 | AF217515 | Hs.283532 | uncharacterized bone marrow protein BM03 | 2.06 | 1.09 |
| 424098 | AF077374 | Hs.139322 | small proline-rich protein 3 | 2.47 | 3.64 |
| 437553 | AI829935 | Hs.130497 | ESTs, Weakly similar to MAT8_HUMAN CHLOR | 2.09 | 0.91 |
| 428036 | AW068302 | | Homo sapiens mRNA for caldesmon, 3′ UTR | 2.71 | 4.58 |
| 446839 | BE091926 | Hs.16244 | mitotic spindle coiled-coil related prot | 3.18 | 2.33 |
| 448479 | H96115 | Hs.21293 | UDP-N-acteylglucosamine pyrophosphorylas | 2.61 | 1.81 |
| 412059 | AA317962 | Hs.249721 | ESTs, Moderately similar to PC4259 ferri | 1.90 | 2.02 |
| 401241 | AB028989 | | mitogen-activated protein kinase 8 inter | 1.26 | 2.55 |
| 408741 | M73720 | Hs.646 | carboxypeptidase A3 (mast cell) | 2.12 | 1.23 |
| 415989 | AI267700 | | ESTs | 1.60 | 1.00 |
| 431070 | AW408164 | Hs.249184 | transcription factor 19 (SC1) | 1.73 | 1.50 |
| 452140 | AB007928 | Hs.28169 | KIAA0459 protein | 2.44 | 2.95 |
| 443162 | T49951 | Hs.9029 | DKFZP434G032 protein | 2.98 | 1.00 |
| 435904 | AF261655 | Hs.8910 | 1,2-alpha-mannosidase IC | 2.56 | 1.20 |
| 407379 | AA332127 | Hs.325804 | transcription factor 17 | 2.10 | 1.72 |
| 442712 | BE465168 | Hs.131011 | ESTs | 2.54 | 2.72 |
| 411678 | AI907114 | Hs.71465 | squalene epoxidase | 1.12 | 3.11 |
| 406791 | AI220684 | Hs.347939 | hemoglobin, alpha 2 | 1.69 | 1.38 |
| 431805 | NM_014053 | Hs.270594 | FLVCR protein | 1.92 | 2.05 |
| 438414 | AA806794 | Hs.131511 | ESTs | 1.04 | 2.15 |
| 413924 | AL119964 | Hs.75616 | seladin-1 | 1.69 | 2.05 |
| 437679 | NM_014214 | Hs.5753 | inositol(myo)-1(or 4)-monophosphatase 2 | 2.27 | 2.26 |
| 445911 | AI985987 | Hs.145645 | ESTs, Moderately similar to ALU1_HUMAN A | 1.42 | 2.74 |
| 408349 | BE546947 | Hs.44276 | homeo box C10 | 1.60 | 2.05 |
| 422545 | X02761 | Hs.287820 | fibronectin 1 | 1.77 | 3.02 |
| 406643 | N77976 | Hs.347939 | hemoglobin, alpha 2 | 1.57 | 1.35 |
| 407228 | M25079 | Hs.155376 | hemoglobin, beta | 1.81 | 1.50 |
| 449644 | AW960707 | Hs.148324 | ESTs | 1.90 | 3.19 |
| 402305 | | | C19000735*: gi\|4508027\|ref\|NP_003414.1\|z | 2.25 | 1.49 |
| 427683 | BE545490 | Hs.15053 | Homo sapiens HCMOGT-1 mRNA for sperm ant | 1.08 | 2.25 |
| 441690 | R81733 | Hs.33106 | ESTs | 1.80 | 2.65 |
| 434487 | AF143867 | Hs.337588 | ESTs, Moderately similar to S65657 alpha | 2.13 | 1.63 |
| 403362 | | | NM_001615*: Homo sapiens actin, gamma 2, | 2.33 | 2.22 |
| 445496 | AB007860 | Hs.12802 | development and differentiation enhancin | 1.12 | 2.60 |
| 425234 | AW152225 | Hs.165909 | ESTs, Weakly similar to I38022 hypotheti | 1.83 | 3.05 |
| 402366 | AV648601 | | apolipoprotein B (including Ag(x) antige | 1.32 | 2.05 |
| 427254 | AL121523 | Hs.97774 | ESTs | 2.44 | 1.00 |
| 414533 | AA149060 | Hs.296100 | ESTs | 1.70 | 2.06 |
| 430157 | BE348706 | Hs.278543 | ESTs | 2.54 | 3.00 |
| 413433 | NM_003199 | Hs.326198 | transcription factor 4 | 2.26 | 1.41 |
| 410532 | T53088 | Hs.155376 | hemoglobin, beta | 1.52 | 1.56 |
| 405779 | | | NM_005367: Homo sapiens melanoma antigen, | 2.42 | 1.22 |
| 450455 | AL117424 | Hs.25035 | chloride intracellular channel 4 | 2.48 | 2.13 |
| 414081 | AW969976 | Hs.279009 | matrix Gla protein | 1.81 | 1.53 |
| 414761 | AU077228 | Hs.77256 | enhancer of zeste (Drosophila) homolog 2 | 1.65 | 2.08 |
| 415062 | H45100 | Hs.49753 | uveal autoantigen with coiled coil domai | 1.62 | 3.75 |
| 406317 | | | C2002658*: gi\|6625694\|gb\|AAF19354.1\|AF185 | 1.68 | 2.11 |
| 453259 | R93125 | Hs.124187 | ESTs | 1.08 | 2.25 |
| 445937 | AI452943 | Hs.321231 | UDP-Gal: betaGlcNAc beta 1,4-galactosylt | 1.76 | 1.01 |
| 434370 | AF130988 | Hs.58346 | ectodysplasin 1, anhidrotic receptor | 1.12 | 2.08 |
| 418058 | AW161552 | Hs.83381 | guanine nucleotide binding protein 11 | 2.02 | 2.40 |
| 432331 | W37862 | Hs.274368 | MSTP032 protein | 4.36 | 2.18 |
| 451736 | AW080356 | Hs.23889 | ESTs, Weakly similar to ALU7_HUMAN ALU S | 1.51 | 3.45 |
| 413109 | AW389845 | Hs.110855 | ESTs | 4.34 | 5.98 |
| 413643 | AA130987 | Hs.188727 | ESTs | 1.30 | 2.42 |
| 433217 | AB040914 | Hs.278628 | KIAA1481 protein | 1.70 | 2.49 |
| 435232 | NM_001262 | Hs.4854 | cyclin-dependent kinase inhibitor 2C (p1 | 1.79 | 1.66 |
| 438006 | BE148799 | Hs.127951 | hypothetical protein FLJ14503 | 1.76 | 2.04 |
| 410102 | AW248508 | Hs.279727 | ESTs; homologue of PEM-3 [Ciona savignyi | 2.32 | 2.45 |
| 433656 | AW974941 | Hs.292385 | ESTs, Weakly similar to I78885 serine/th | 1.14 | 2.50 |
| 415714 | NM_002290 | Hs.78672 | laminin, alpha 4 | 2.52 | 1.13 |
| 450282 | AA007655 | Hs.93523 | ESTs | 1.40 | 2.58 |
| 442855 | AI074465 | Hs.133469 | ESTs | 1.54 | 2.20 |
| 432917 | NM_014125 | Hs.241517 | PRO0327 protein | 2.24 | 3.03 |
| 429041 | AJ132820 | Hs.194768 | a disintegrin and metalloproteinase doma | 1.61 | 2.60 |
| 442807 | AL049274 | Hs.8736 | Homo sapiens mRNA; cDNA DKFZp564H203 (fr | 1.73 | 1.19 |
| 427719 | AI393122 | Hs.134726 | ESTs | 1.46 | 2.00 |
| 408778 | AI500519 | Hs.63382 | hypothetical protein PRO2714 | 1.46 | 2.58 |
| 418870 | AF147204 | Hs.89414 | chemokine (C-X-C motif), receptor 4 (fus | 1.40 | 2.03 |
| 424425 | AB031480 | Hs.146824 | SPR1 protein | 1.60 | 1.11 |
| 445391 | T92576 | Hs.191168 | ESTs | 1.69 | 2.40 |
| 446899 | NM_005397 | Hs.16426 | podocalyxin-like | 1.22 | 2.42 |
| 420996 | AK001927 | Hs.100895 | hypothetical protein FLJ10462 | 2.68 | 2.98 |
| 424909 | S78187 | Hs.153752 | cell division cycle 25B | 2.18 | 1.33 |
| 413593 | AA205248 | | gb: zq78c12.r1 Stratagene hNT neuron (937 | 1.69 | 2.00 |
| 408734 | AW264996 | Hs.254299 | ESTs | 2.06 | 2.94 |
| 413880 | AI660842 | Hs.110915 | interleukin 22 receptor | 1.24 | 2.20 |
| 437063 | AA351109 | Hs.5437 | Tax1 (human T-cell leukemia virus type I | 1.78 | 2.60 |
| 418044 | AI640532 | Hs.119830 | ESTs, Weakly similar to ALUF_HUMAN !!!! | 1.54 | 2.53 |
| 441971 | W27060 | Hs.265855 | ESTs | 1.62 | 2.13 |
| 450401 | AW959281 | Hs.8184 | ESTs | 1.42 | 2.30 |
| 440157 | AA868350 | Hs.343636 | ESTs | 1.38 | 2.60 |
| 457587 | AA992841 | Hs.27263 | KIAA1458 protein | 1.47 | 2.42 |
| 440707 | BE256751 | Hs.22867 | Homo sapiens cDNA: FLJ22073 fis, clone H | 1.18 | 2.10 |
| 402241 | | | Target Exon | 2.58 | 2.52 |
| 413428 | AA430155 | Hs.151343 | KIAA1524 protein | 1.22 | 2.45 |
| 416735 | R11275 | Hs.194485 | ESTs | 1.14 | 2.14 |
| 421582 | AI910275 | | trefoil factor 1 (breast cancer, estroge | 1.25 | 1.03 |
| 431031 | AA830335 | Hs.105273 | ESTs | 2.35 | 2.95 |
| 433336 | AF017986 | Hs.31386 | secreted frizzled-related protein 2 | 3.72 | 1.00 |
| 420786 | AW296466 | Hs.43628 | deleted in lymphocytic leukemia, 2 | 1.23 | 2.60 |
| 401335 | | | Target Exon | 1.18 | 1.68 |
| 417670 | R07785 | | gb: yf15c06.r1 Soares fetal liver spleen | 1.56 | 2.00 |
| 406314 | | | C14001020: gi\|12597441\|gb\|AAG60049.1\|AF31 | 1.60 | 3.08 |
| 458981 | AW968318 | Hs.285996 | hypothetical protein FLJ23375 | 1.70 | 2.50 |
| 417509 | AA203414 | Hs.42009 | ESTs | 1.82 | 2.05 |
| 452732 | BE300078 | Hs.80449 | Homo sapiens, clone IMAGE: 3535294, mRNA, | 1.34 | 1.37 |
| 418678 | NM_001327 | Hs.87225 | cancer/testis antigen (NY-ESO-1) | 1.37 | 1.02 |
| 457871 | AI168278 | | ESTs | 1.20 | 2.19 |
| 444163 | AI126098 | | gb: qc54g07.x1 Soares_placent_8to9weeks_{—} | 1.43 | 1.22 |
| 413276 | Z24725 | Hs.75260 | mitogen inducible 2 | 1.78 | 2.28 |
| 421097 | AI280112 | Hs.125232 | Homo sapiens cDNA FLJ13266 fis, clone OV | 2.55 | 2.60 |
| 417151 | AA194055 | Hs.293858 | ESTs | 1.68 | 1.67 |
| 453556 | AA425414 | Hs.33287 | nuclear factor I/B | 2.06 | 2.40 |
| 440859 | AW070865 | Hs.346390 | ESTs | 1.12 | 1.70 |
| 420629 | AW204343 | Hs.156823 | ESTs, Weakly similar to T30868 RhoA-bind | 1.21 | 2.38 |
| 422363 | T55979 | Hs.115474 | replication factor C (activator 1) 3 (38 | 1.58 | 2.15 |
| 434831 | AA248060 | Hs.273397 | KIAA0710 gene product | 1.69 | 1.78 |
| 412055 | AA099907 | Hs.271806 | ESTs, Weakly similar to ALU1_HUMAN ALU S | 1.36 | 2.65 |
| 445468 | AW450439 | | ESTs | 1.52 | 2.50 |
| 444550 | BE250716 | Hs.87614 | ESTs | 1.30 | 2.28 |
| 417259 | AW903838 | Hs.81800 | chondroitin sulfate proteoglycan 2 (vers | 1.50 | 3.02 |
| 430233 | AW367902 | Hs.236443 | Homo sapiens mRNA; cDNA DKFZp564N1063 (f | 1.24 | 2.95 |
| 413444 | BE141019 | | gb: MR0-HT0067-201099-002-b10 HT0067 Homo | 1.68 | 2.80 |
| 433844 | AA610175 | Hs.179647 | Homo sapiens cDNA FLJ12195 fis, clone MA | 1.49 | 2.01 |
| 427055 | AI301740 | Hs.173381 | dihydropyrimidinase-like 2 | 1.11 | 2.58 |
| 454244 | R51604 | Hs.300842 | KIAA1608 protein | 1.00 | 2.02 |
| 429503 | AA394183 | Hs.26873 | ESTs | 2.58 | 4.08 |
| 422940 | BE077458 | | gb: RC1-BT0606-090500-015-b04 BT0606 Homo | 3.48 | 2.46 |
| 407949 | W21874 | Hs.247057 | ESTs, Weakly similar to 2109260A B cell | 3.13 | 1.48 |
| 437312 | AA809350 | Hs.246180 | ESTs | 1.10 | 2.05 |
| 449426 | T92251 | Hs.198882 | ESTs | 1.22 | 2.08 |
| 447620 | AW290951 | Hs.224965 | ESTs | 1.80 | 2.18 |
| 444700 | NM_003645 | Hs.11729 | fatty-acid-Coenzyme A ligase, very long- | 1.72 | 2.81 |
| 436258 | AW867491 | Hs.107125 | plasmalemma vesicle associated protein | 1.99 | 1.80 |
| 415712 | AW249188 | Hs.169577 | Homo sapiens cDNA FLJ14743 fis, clone NT | 1.66 | 2.98 |
| 432877 | AW974111 | Hs.292477 | ESTs | 1.63 | 2.79 |
| 412085 | AW891667 | | gb: CM3-NT0089-110500-179-h09 NT0089 Homo | 1.40 | 2.08 |
| 431421 | AW969118 | Hs.108144 | ESTs, Weakly similar to unnamed protein | 1.96 | 3.58 |
| 409714 | AW367812 | Hs.199961 | ESTs, Weakly similar to ALU7_HUMAN ALU S | 1.24 | 2.10 |
| 423013 | AW875443 | Hs.22209 | secreted modular calcium-binding protein | 1.49 | 2.09 |
| 422663 | AW500087 | Hs.119014 | zinc finger protein 175 | 1.01 | 2.78 |
| 439737 | AI751438 | Hs.41271 | Homo sapiens mRNA full length insert cDN | 1.54 | 2.57 |
| 413196 | AA127386 | | gb: zn90d09.r1 Stratagene lung carcinoma | 1.04 | 2.18 |
| 439349 | AI660898 | Hs.195602 | ESTs | 2.03 | 2.43 |
| 443005 | AI027184 | Hs.200918 | ESTs | 1.42 | 2.10 |
| 424762 | AL119442 | Hs.183684 | eukaryotic translation initiation factor | 2.58 | 3.43 |
| 427373 | AB007972 | Hs.130760 | myosin phosphatase, target subunit 2 | 2.08 | 1.70 |
| 413916 | N49813 | Hs.75615 | apolipoprotein C-II | 0.70 | 0.42 |
| 418332 | R34976 | Hs.78293 | ESTs | 2.74 | 2.43 |
| 426552 | BE297660 | Hs.170328 | moesin | 1.28 | 1.52 |
| 456583 | AF179897 | Hs.104105 | Meis (mouse) homolog 2 | 1.42 | 2.02 |
| 447214 | AI367288 | Hs.273621 | Homo sapiens cDNA: FLJ21350 fis, clone C | 1.14 | 2.10 |
| 449254 | W26908 | Hs.172762 | ESTs | 2.04 | 2.50 |
| 443651 | W22152 | Hs.282929 | ESTs | 2.94 | 3.08 |
| 421021 | AA808018 | Hs.109302 | ESTs | 1.41 | 2.04 |
| 419741 | NM_007019 | Hs.93002 | ubiquitin carrier protein E2-C | 1.61 | 1.21 |
| 432027 | AL096678 | Hs.272353 | KIAA0957 protein | 1.70 | 2.76 |
| 452688 | AA721140 | Hs.49930 | ESTs, Weakly similar to putative p150 [H | 1.80 | 2.95 |
| 417042 | C75563 | Hs.113029 | ribosomal protein S25 | 2.22 | 3.20 |
| 443574 | U83993 | Hs.321709 | purinergic receptor P2X, ligand-gated io | 1.21 | 2.51 |
| 429372 | AA451859 | Hs.99253 | ESTs | 1.28 | 2.43 |
| 424290 | AA338396 | | gb: EST43386 Fetal brain I Homo sapiens c | 1.82 | 2.03 |
| 428518 | AW969656 | | gb: EST381733 MAGE resequences, MAGK Homo | 1.72 | 2.52 |
| 455649 | BE065051 | | gb: RC1-BT0313-110500-017-c04 BT0313 Homo | 1.65 | 3.03 |
| 414665 | AA160873 | Hs.332053 | serum amyloid A1 | 1.36 | 1.08 |
| 418298 | AA256014 | Hs.86682 | Homo sapiens cDNA: FLJ21578 fis, clone C | 1.04 | 2.03 |
| 429655 | U48959 | Hs.211582 | myosin, light polypeptide kinase | 4.94 | 4.34 |
| 433924 | AA618304 | Hs.258785 | ESTs | 1.44 | 2.40 |
| 452683 | AI089575 | Hs.9071 | progesterone membrane binding protein | 1.48 | 2.48 |
| 439437 | AI207788 | Hs.343628 | sialyltransferase 4B (beta-galactosidase | 1.36 | 2.33 |
| 432314 | AA533447 | Hs.312989 | ESTs | 0.96 | 2.78 |
| 400881 | | | NM_025080: Homo sapiens hypothetical prot | 1.70 | 3.15 |
| 426477 | AA379464 | | gb: EST92386 Skin tumor I Homo sapiens cD | 2.01 | 2.37 |
| 454741 | BE154396 | | gb: CM2-HT0342-091299-050-b05 HT0342 Homo | 2.12 | 3.44 |
| 423977 | AA333232 | | gb: EST37283 Embryo, 8 week I Homo sapien | 1.38 | 2.13 |
| 450396 | AU077002 | Hs.24950 | regulator of G-protein signalling 5 | 2.13 | 3.28 |
| 431842 | NM_005764 | Hs.271473 | epithelial protein up-regulated in carci | 1.90 | 2.23 |
| 415157 | D63257 | | gb: HUM514B08B Clontech human placenta po | 1.29 | 2.90 |
| 418236 | AW994005 | Hs.337534 | ESTs | 1.74 | 2.37 |
| 454390 | AB020713 | Hs.56966 | KIAA0906 protein | 1.47 | 1.38 |
| 436143 | AA705245 | Hs.192189 | ESTs | 1.46 | 2.45 |
| 436251 | BE515065 | Hs.296585 | nucleolar protein (KKE/D repeat) | 1.43 | 2.07 |
| 450735 | AI732321 | | SRY (sex determining region Y)-box 4 | 1.36 | 2.02 |
| 420136 | AW801090 | Hs.195851 | actin, alpha 2, smooth muscle, aorta | 2.70 | 1.68 |
| 447100 | AI361801 | Hs.167130 | hypothetical protein | 1.66 | 2.03 |
| 453577 | AL043049 | | gb: DKFZp434A1523_r1 434 (synonym: htes3) | 1.41 | 2.75 |
| 408522 | AI541214 | Hs.46320 | Small proline-rich protein SPRK [human, | 1.59 | 1.38 |
| 412632 | AL120379 | Hs.74294 | aldehyde dehydrogenase 7 family, member | 1.81 | 2.51 |
| 423291 | NM_004129 | Hs.126590 | guanylate cyclase 1, soluble, beta 2 | 1.54 | 2.83 |
| 456172 | R99050 | | gb: yq65c02.r1 Soares fetal liver spleen | 1.46 | 2.68 |
| 452123 | AI267615 | Hs.38022 | ESTs | 1.24 | 1.93 |
| 433900 | AA721668 | Hs.257761 | ESTs | 1.78 | 2.66 |
| 408436 | R31954 | Hs.7885 | phosphatidylinositol binding clathrin as | 1.21 | 2.35 |
| 417123 | BE326521 | Hs.159450 | ESTs | 1.46 | 2.09 |
| 436023 | T81819 | Hs.302251 | ESTs | 2.68 | 2.70 |
| 454150 | AA131893 | Hs.154088 | hypothetical protein FLJ22756 | 1.40 | 2.50 |
| 444094 | AI695764 | Hs.202394 | ESTs | 1.28 | 4.03 |
| 429176 | AW975021 | Hs.193800 | ESTs | 1.08 | 2.53 |
| 422259 | AA307584 | | gb: EST178498 Colon carcinoma (HCC) cell | 1.80 | 3.03 |
| 451164 | AA015912 | Hs.60659 | ESTs, Weakly similar to T46471 hypotheti | 1.47 | 1.17 |
| 417501 | AL041219 | Hs.82222 | sema domain, immunoglobulin domain (lg), | 2.92 | 2.70 |
| 455642 | BE063965 | | gb: QV3-BT0296-140200-085-h01 BT0296 Homo | 1.70 | 2.70 |
| 443387 | BE139135 | Hs.254629 | ESTs | 1.48 | 2.32 |
| 420995 | AA282495 | Hs.89014 | ESTs | 1.45 | 1.51 |
| 407329 | AA576061 | Hs.269834 | ESTs, Weakly similar to ALUD_HUMAN !!!! | 1.13 | 2.38 |
| 438797 | C16161 | Hs.283040 | hypothetical protein PRO2543 | 0.99 | 2.75 |
| 443357 | AW016773 | | low molecular mass ubiquinone-binding pr | 1.60 | 2.08 |
| 412656 | AF006011 | Hs.74375 | dishevelled 1 (homologous to Drosophila | 1.32 | 1.13 |
| 427377 | AU077029 | Hs.177543 | antigen identified by monoclonal antibod | 1.24 | 0.79 |
| 412200 | R08110 | Hs.187462 | ESTs, Weakly similar to I38022 hypotheti | 1.35 | 1.54 |
| 432586 | AA568548 | | ESTs | 1.50 | 2.25 |
| 411590 | T96183 | | gb: ye09f07.s1 Stratagene lung (937210) H | 1.22 | 2.53 |
| 422672 | X12784 | Hs.119129 | collagen, type IV, alpha 1 | 2.27 | 2.20 |
| 420256 | U84722 | Hs.76206 | cadherin 5, type 2, VE-cadherin (vascula | 1.59 | 2.11 |
| 419900 | AI469960 | Hs.170698 | ESTs | 1.30 | 2.68 |
| 410805 | AW804742 | Hs.84264 | acidic protein rich in leucines | 1.16 | 2.28 |
| 452560 | BE077084 | Hs.99969 | ESTs | 1.44 | 2.58 |
| 448429 | D17408 | Hs.21223 | calponin 1, basic, smooth muscle | 4.02 | 1.00 |
| 424436 | AW818428 | Hs.4953 | golgi autoantigen, golgin subfamily a, 3 | 1.10 | 2.00 |
| 447400 | AK000322 | Hs.18457 | hypothetical protein FLJ20315 | 1.45 | 1.28 |
| 422522 | AI023428 | Hs.34549 | ESTs, Highly similar to S94541 1 clone 4 | 1.82 | 2.10 |
| 443696 | AW607444 | Hs.134622 | ESTs | 1.98 | 2.01 |
| 436094 | AI798701 | Hs.222222 | ESTs | 1.34 | 2.40 |
| 420168 | AF217508 | Hs.95594 | serine carboxypeptidase vitellogenic-lik | 1.58 | 2.45 |
| 430325 | AF004562 | Hs.239356 | syntaxin binding protein 1 | 1.34 | 2.43 |
| 439022 | AA356599 | Hs.173904 | ESTs | 2.76 | 2.40 |
| 420563 | AA278327 | Hs.136237 | ESTs, Moderately similar to Y140_HUMAN H | 1.78 | 2.65 |
| 429494 | AA769365 | Hs.126058 | ESTs | 1.50 | 2.40 |
| 420689 | H79979 | Hs.88678 | ESTs | 1.26 | 2.28 |
| 448988 | Y09763 | Hs.22785 | gamma-aminobutyric acid (GABA) A recepto | 1.72 | 1.29 |
| 439943 | AW083789 | Hs.124620 | ESTs | 1.45 | 2.84 |
| 442300 | AI765908 | Hs.129166 | ESTs | 1.24 | 2.35 |
| 449614 | AI989490 | Hs.197703 | ESTs | 1.12 | 2.22 |
| 444363 | AI142827 | Hs.143656 | ESTs | 1.32 | 2.08 |
| 424479 | AF064238 | Hs.149098 | smoothelin | 1.59 | 1.10 |
| 437321 | AA768966 | Hs.292026 | ESTs, Weakly similar to 2109260A B cell | 1.28 | 2.07 |
| 431926 | AW972724 | | gb: EST384816 MAGE resequences, MAGL Homo | 1.52 | 2.63 |
| 433640 | AW390125 | Hs.240443 | Homo sapiens cDNA: FLJ23538 fis, clone L | 1.69 | 1.30 |
| 415901 | H08396 | Hs.76118 | ubiquitin carboxyl-terminal esterase L1 | 1.71 | 2.19 |
| 437199 | AL110175 | Hs.306337 | Homo sapiens mRNA; cDNA DKFZp564H0616 (f | 1.48 | 2.50 |
| 457450 | AW294163 | Hs.146127 | ESTs | 1.07 | 2.60 |
| 456678 | AF141305 | Hs.173736 | ancient ubiquitous protein 1 | 1.44 | 2.35 |
| 451079 | AI827988 | Hs.240728 | ESTs, Moderately similar to PC4259 ferri | 0.95 | 3.00 |
| 405944 | | | Target Exon | 1.48 | 2.45 |
| 408877 | AA479033 | Hs.130315 | ESTs, Weakly similar to A47582 B-cell gr | 1.38 | 2.20 |
| 446682 | AW205632 | Hs.211198 | ESTs | 1.38 | 3.00 |
| 431380 | AW610282 | Hs.291003 | ESTs | 1.43 | 2.64 |
| 442027 | AI652926 | Hs.128395 | ESTs | 1.18 | 2.43 |
| 423578 | AW960454 | Hs.222830 | ESTs | 1.56 | 2.18 |
| 441495 | AW294603 | Hs.127039 | ESTs | 2.80 | 1.73 |
| 417900 | BE250127 | Hs.82906 | CDC20 (cell division cycle 20, S. cerevi | 1.36 | 1.18 |
| 443949 | AW827419 | Hs.235070 | ESTs | 1.30 | 2.28 |
| 440495 | AA887212 | Hs.14161 | hypothetical protein DKFZp434I1930 | 1.74 | 2.78 |
| 449948 | R19156 | Hs.20798 | ESTs | 1.12 | 2.23 |
| 439564 | W77911 | Hs.110006 | ESTs | 1.34 | 2.85 |
| 423225 | AA852604 | Hs.125359 | Thy-1 cell surface antigen | 1.24 | 1.09 |
| 436139 | AA765786 | Hs.120936 | ESTs | 1.30 | 2.10 |
| 456968 | AI174861 | Hs.190623 | ESTs | 1.14 | 2.15 |
| 437191 | NM_006846 | Hs.331555 | serine protease inhibitor, Kazal type, 5 | 1.21 | 2.18 |
| 411652 | AW855393 | | gb: CM3-CT0275-191099-024-f10 CT0275 Homo | 1.85 | 1.94 |
| 420732 | AA789133 | Hs.88650 | ESTs | 1.66 | 2.71 |
| 409291 | AW373472 | | gb: RC3-BT0523-181299-011-d12 BT0523 Homo | 1.56 | 2.30 |
| 424415 | NM_001975 | Hs.146580 | enolase 2, (gamma, neuronal) | 1.56 | 1.39 |
| 424927 | AW973666 | Hs.153850 | hypothetical protein C321D2.4 | 1.58 | 1.24 |
| 450946 | AA374569 | Hs.127698 | ESTs, Moderately similar to 2109260A B c | 1.02 | 2.25 |
| 428423 | AU076517 | Hs.184276 | solute carrier family 9 (sodium/hydrogen | 1.70 | 2.39 |
| 415361 | F06724 | | gb: HSC1IG021 normalized infant brain cDN | 1.34 | 2.40 |
| 406490 | | | C5001926: gi\|7511572\|pir∥T42245 probable | 1.28 | 2.40 |
| 410855 | X97795 | Hs.66718 | RAD54 (S. cerevisiae)-like | 1.52 | 2.00 |
| 440010 | AA534930 | Hs.127236 | hypothetical protein FLJ12879 | 1.12 | 2.20 |
| 429508 | AW369620 | Hs.33944 | ESTs, Weakly similar to ALU1_HUMAN ALU S | 1.33 | 2.28 |
| 426340 | Z97989 | Hs.169370 | FYN oncogene related to SRC, FGR, YES | 1.88 | 2.18 |
| 416889 | AW250318 | Hs.80395 | mal, T-cell differentiation protein | 1.65 | 1.16 |
| 451870 | AI820991 | Hs.8377 | ESTs | 1.24 | 2.03 |
| 444091 | AV647924 | Hs.282376 | ESTs | 1.05 | 2.13 |
| 410793 | AW581906 | Hs.66392 | intersectin 1 (SH3 domain protein) | 2.00 | 3.13 |
| 452222 | AW806287 | Hs.21432 | SEX gene | 1.25 | 1.10 |
| 433010 | AW970018 | | gb: EST382097 MAGE resequences, MAGK Homo | 1.36 | 2.41 |
| 432674 | AA641092 | Hs.257339 | ESTs, Weakly similar to I38022 hypotheti | 1.14 | 2.03 |
| 438855 | AW946276 | Hs.6441 | Homo sapiens mRNA; cDNA DKFZp586J021 (fr | 2.24 | 1.77 |
| 448718 | AA220235 | Hs.153959 | hypothetical protein MGC15436 | 1.52 | 2.65 |
| 402685 | | | Target Exon | 2.04 | 2.46 |
| 424528 | AW073971 | Hs.238954 | ESTs, Weakly similar to KIAA1204 protein | 1.66 | 2.05 |
| 422068 | AI807519 | Hs.104520 | Homo sapiens cDNA FLJ13694 fis, clone PL | 1.89 | 4.98 |
| 451225 | AI433694 | Hs.293608 | ESTs | 1.79 | 2.70 |
| 441078 | AI453268 | Hs.323409 | Homo sapiens cDNA FLJ14113 fis, clone MA | 1.44 | 2.58 |
| 409406 | H83092 | Hs.49605 | ESTs | 1.38 | 2.05 |
| 422297 | AW961290 | | p30 DBC protein | 1.20 | 2.73 |
| 408711 | AW376061 | Hs.63335 | ESTs, Moderately similar to A46010 X-lin | 1.20 | 2.08 |
| 426696 | AW363332 | Hs.171844 | Homo sapiens cDNA: FLJ22296 fis, clone H | 1.35 | 2.68 |
| 417324 | AW265494 | | ESTs | 1.68 | 1.25 |
| 408283 | BE141579 | | gb: QV2-HT0083-071299-018-b05 HT0083 Homo | 1.25 | 2.65 |
| 415166 | NM_003652 | Hs.78068 | carboxypeptidase Z | 1.34 | 1.09 |
| 406300 | | | Target Exon | 1.61 | 2.47 |
| 411880 | AW872477 | | gb: hm30f03.x1 NCI_CGAP_Thy4 Homo sapiens | 3.60 | 4.03 |
| 422287 | F16365 | Hs.114346 | cytochrome c oxidase subunit Vlla polype | 2.16 | 1.44 |
| 422567 | AF111178 | Hs.118407 | glypican 6 | 1.57 | 2.03 |
| 436855 | AA732624 | Hs.165852 | ESTs | 1.08 | 2.75 |
| 403536 | | | Target Exon | 0.93 | 2.13 |
| 447733 | AF157482 | Hs.19400 | MAD2 (mitotic arrest deficient, yeast, h | 1.18 | 1.07 |
| 417117 | N46778 | | gb: yy52b02.r1 Soares_multiple_sclerosis_{—} | 1.70 | 2.85 |
| 411690 | AA669253 | Hs.136075 | RNA, U2 small nuclear | 2.12 | 2.78 |
| 443243 | AI452496 | Hs.132056 | ESTs | 1.15 | 2.83 |
| 423074 | AL109963 | | FSH primary response (LRPR1, rat) homolo | 1.37 | 1.43 |
| 408916 | AW295232 | Hs.429 | ATP synthase, H transporting, mitochondr | 1.63 | 2.23 |
| 449799 | AI143466 | Hs.125060 | ESTs | 1.40 | 2.08 |
| 415378 | T16964 | | gb: NIB2079-5R Normalized infant brain, B | 1.88 | 1.85 |
| 431089 | BE041395 | | ESTs, Weakly similar to unknown protein | 1.57 | 2.57 |
| 434959 | AW974949 | Hs.186564 | ESTs, Weakly similar to I38022 hypotheti | 1.30 | 2.30 |
| 416311 | D80529 | | gb: HUM081H05B Human fetal brain (TFujiwa | 1.58 | 4.35 |
| 444614 | R44284 | Hs.2730 | heterogeneous nuclear ribonucleoprotein | 1.88 | 2.98 |
| 456206 | NM_006895 | Hs.81182 | histamine N-methyltransferase | 1.24 | 2.08 |
| 410583 | AW770280 | Hs.36258 | ESTs, Moderately similar to JC5238 galac | 1.56 | 4.33 |
| 430410 | AF099144 | Hs.334455 | tryptase beta 1 | 1.91 | 1.58 |
| 408139 | AA451966 | | RAB9-like protein | 1.42 | 2.14 |
| 432621 | AI298501 | Hs.12807 | ESTs, Weakly similar to T46428 hypotheti | 2.08 | 1.94 |
| 441584 | AW148329 | Hs.175208 | ESTs | 1.12 | 2.05 |
| 445940 | D60438 | Hs.34779 | ESTs | 1.86 | 2.70 |
| 453022 | AA031499 | Hs.118489 | ESTs | 2.02 | 1.75 |
| 444008 | BE544855 | Hs.236572 | ESTs, Weakly similar to SFR4_HUMAN SPLIC | 1.54 | 1.29 |
| 442994 | AI026718 | Hs.16954 | ESTs | 3.60 | 3.78 |
| 402085 | | | C18000504*: gi\|2627436\|gb\|AAB86683.1\| (AF | 1.36 | 2.53 |
| 411918 | AW876354 | | gb: PM4-PT0019-141299-009-F08 PT0019 Homo | 2.00 | 2.63 |
| 455508 | AW976165 | | gb: EST388274 MAGE resequences, MAGN Homo | 1.70 | 3.04 |
| 426106 | AI678765 | Hs.21812 | ESTs | 1.49 | 2.11 |
| 425131 | BE252230 | Hs.99163 | ESTs | 2.04 | 2.65 |
| 440325 | NM_003812 | Hs.7164 | a disintegrin and metalloproteinase doma | 1.17 | 2.55 |
| 420447 | AA687306 | Hs.88448 | ESTs | 1.66 | 2.58 |
| 428055 | AA420564 | Hs.101760 | ESTs | 1.08 | 2.15 |
| 422110 | AI376736 | Hs.111779 | secreted protein, acidic, cysteine-rich | 1.76 | 1.82 |
| 438581 | AW977766 | Hs.292133 | ESTs, Moderately similar to I78885 serin | 1.08 | 2.10 |
| 403290 | | | C1000101*: gi\|4758212\|ref\|NP_004411.1\| d | 0.97 | 2.48 |
| 408175 | W29089 | Hs.19066 | hypothetical protein DKFZp667O2416 | 1.42 | 1.41 |
| 432390 | AA936177 | Hs.274460 | olfactory receptor, family 5, subfamily | 1.26 | 2.05 |
| 443441 | AW291196 | Hs.92195 | ESTs | 1.52 | 2.13 |
| 419925 | AA159850 | Hs.93765 | lipoma HMGIC fusion partner | 1.72 | 2.80 |
| 445256 | AI858635 | Hs.144763 | ESTs | 1.97 | 3.33 |
| 456381 | AA236606 | | gb: zr99b10.r1 NCI_CGAP_GCB1 Homo sapiens | 1.16 | 1.95 |
| 422433 | AA310560 | Hs.153746 | hypothetical protein FLJ22490 | 1.06 | 2.20 |
| 432529 | AI989507 | Hs.162245 | ESTs | 1.36 | 2.25 |
| 424951 | AW964082 | | gb: EST376155 MAGE resequences, MAGH Homo | 2.22 | 2.58 |
| 420785 | H89633 | Hs.191346 | ESTs | 1.26 | 2.15 |
| 411347 | AW838126 | | gb: QV2-LT0051-240300-097-f01 LT0051 Homo | 1.38 | 2.38 |
| 438742 | AW204126 | Hs.196543 | ESTs | 1.10 | 2.30 |
| 414900 | AW452420 | Hs.248678 | ESTs | 2.01 | 3.08 |
| 443284 | AI369813 | Hs.64783 | ESTs, Weakly similar to T42705 hypotheti | 0.66 | 0.43 |
| 402049 | | | Target Exon | 2.28 | 2.00 |
| 429400 | AW604940 | Hs.201668 | transcription factor 20 (AR1) | 1.16 | 2.00 |
| 423916 | AW993496 | Hs.17235 | Homo sapiens clone TCCCIA00176 mRNA sequ | 1.59 | 1.05 |
| 432495 | AW973537 | Hs.186734 | ESTs, Weakly similar to I61746 pheromone | 1.50 | 2.05 |
| 414840 | R27319 | Hs.23823 | hairy/enhancer-of-split related with YRP | 1.89 | 2.09 |
| 428711 | R46414 | Hs.56828 | trinucleotide repeat containing 5 | 1.77 | 1.83 |
| 448609 | AW139420 | Hs.7972 | KIAA0871 protein | 1.14 | 2.26 |
| 443859 | NM_013409 | Hs.9914 | follistatin | 1.17 | 1.05 |
| 411141 | AW819561 | | gb: RC5-ST0293-140200-013-G04 ST0293 Homo | 1.44 | 2.40 |
| 440116 | AI798851 | Hs.266959 | hemoglobin, gamma G | 1.18 | 2.08 |
| 417944 | AU077196 | Hs.82985 | collagen, type V, alpha 2 | 2.10 | 1.37 |
| 429640 | U83508 | Hs.2463 | angiopoietin 1 | 1.92 | 2.98 |
| 410064 | X53416 | Hs.195464 | filamin A, alpha (actin-binding protein- | 1.51 | 1.29 |
| 458218 | AI435179 | Hs.126820 | ESTs | 1.49 | 1.15 |
| 443114 | AI033377 | Hs.153298 | ESTs | 1.38 | 2.05 |
| 427788 | AA412397 | Hs.116858 | ESTs | 1.45 | 1.85 |
| 435913 | W95006 | Hs.269559 | ESTs, Weakly similar to S65657 alpha-1C- | 1.63 | 3.90 |
| 457949 | W69171 | Hs.71741 | ESTs, Highly similar to I38945 melanoma | 1.01 | 2.00 |
| 419203 | AA488719 | Hs.190151 | ESTs | 1.94 | 2.45 |
| 412510 | AI056689 | Hs.133538 | ESTs, Weakly similar to ALU1_HUMAN ALU S | 1.91 | 2.20 |
| 413885 | BE177442 | | gb: RC1-HT0595-200400-012-f01 HT0595 Homo | 1.48 | 2.80 |
| 426239 | AA669615 | Hs.214226 | ESTs | 1.36 | 2.50 |
| 408866 | AW292096 | Hs.255036 | ESTs | 1.93 | 2.92 |
| 412857 | AI703484 | Hs.128052 | ESTs | 1.72 | 1.60 |
| 427340 | BE167242 | Hs.47099 | hypothetical protein FLJ21212 | 1.46 | 2.13 |
| 412902 | BE008018 | | gb: QV0-BN0147-290400-214-c01 BN0147 Homo | 0.90 | 2.05 |
| 451141 | AW772713 | Hs.247186 | ESTs | 2.38 | 3.95 |
| 412626 | AA114945 | Hs.151839 | ESTs | 1.75 | 2.15 |
| 405667 | | | Target Exon | 2.62 | 3.79 |
| 417777 | AI823763 | Hs.7055 | ESTs, Weakly similar to I78885 serine/th | 1.24 | 2.08 |
| 401400 | | | Target Exon | 1.16 | 1.90 |
| 426796 | S78234 | Hs.172405 | cell division cycle 27 | 2.14 | 1.63 |
| 435046 | AA662772 | Hs.174330 | ESTs, Weakly similar to ALU1_HUMAN ALU S | 1.14 | 2.28 |
| 448401 | AI498509 | Hs.346254 | ESTs | 2.50 | 2.83 |
| 450832 | AW970602 | Hs.105421 | ESTs | 0.55 | 0.39 |
| 441057 | AL043897 | Hs.126483 | ESTs | 1.08 | 2.13 |
| 438725 | AA815163 | Hs.127307 | ESTs | 1.31 | 2.65 |
| 450062 | AW001043 | Hs.200854 | ESTs | 1.30 | 2.48 |
| 441214 | AI820648 | Hs.129136 | ESTs | 1.43 | 1.71 |
| 431723 | AW058350 | Hs.16762 | Homo sapiens mRNA; cDNA DKFZp564B2062 (f | 1.22 | 2.30 |
| 414907 | X90725 | Hs.77597 | polo (Drosophia)-like kinase | 1.35 | 1.49 |
| 423622 | BE154847 | | gb: PM1-HT0345-121199-001-d05 HT0345 Homo | 1.57 | 2.30 |
| 450835 | BE262773 | Hs.25584 | hypothetical protein FLJ10767 | 1.40 | 1.12 |
| 444014 | AI095718 | Hs.135015 | ESTs | 2.30 | 1.78 |
| 431603 | AA807955 | Hs.325984 | EST | 1.26 | 2.03 |
| 408697 | AW419069 | Hs.209670 | ESTs | 1.35 | 2.60 |
| 444312 | R44007 | | ESTs | 1.95 | 2.07 |
| 404286 | | | C6001909: gi\|704441\|dbj\|BAA18909.1\|(D298 | 2.29 | 2.22 |
| 438813 | M27346 | | gb: Homo sapiens (clone HGP09/HGP32) T ce | 1.03 | 2.43 |
| 445534 | AL038823 | Hs.12840 | Homo sapiens germline mRNA sequence | 1.00 | 2.16 |
| 426046 | AA833655 | Hs.206868 | Homo sapiens cDNA FLJ14056 fis, clone HE | 1.59 | 2.73 |
| 451907 | AI822065 | Hs.50749 | ESTs, Moderately similar to ALU7_HUMAN A | 1.74 | 2.65 |
| 418796 | AA228351 | Hs.34060 | ESTs | 1.28 | 2.12 |
| 422431 | AI769410 | Hs.221461 | ESTs | 1.80 | 3.58 |
| 417557 | AA225622 | Hs.293589 | ESTs | 1.32 | 2.14 |
| 455313 | AW894409 | Hs.125472 | ESTs, Moderately similar to KIAA0877 pro | 1.48 | 2.57 |
| 415479 | F10042 | Hs.4840 | ESTs | 1.83 | 2.01 |
| 450433 | AW444538 | Hs.231863 | ESTs | 1.31 | 2.58 |
| 410581 | AA018982 | Hs.125036 | tumor endothelial marker 7 precursor | 1.54 | 1.62 |
| 455407 | AW936813 | | gb: PM2-DT0023-050400-003-b10 DT0023 Homo | 1.32 | 2.15 |
| 417552 | R00916 | Hs.166510 | ESTs | 1.50 | 2.63 |
| 428290 | AI932995 | Hs.183475 | Homo sapiens clone 25061 mRNA sequence | 1.94 | 2.70 |
| 432391 | AI732374 | Hs.339827 | Human DNA sequence from clone RP5-881L22 | 0.96 | 2.38 |
| 456283 | U68162 | Hs.84171 | myeloproliferative leukemia virus oncoge | 1.22 | 2.13 |
| 438535 | L09078 | | gb: Homo sapiens mRNA fragment | 2.14 | 1.95 |
| 416564 | AW795793 | Hs.2575 | Homo sapiens cDNA FLJ12257 fis, clone MA | 2.28 | 1.93 |
| 435200 | AA670310 | Hs.145903 | ESTs | 1.16 | 2.13 |
| 457635 | AV660976 | Hs.3569 | hypothetical protein | 1.37 | 3.10 |
| 444930 | BE185536 | Hs.301183 | molecule possessing ankyrin repeats indu | 0.99 | 2.45 |
| 449319 | AA373630 | Hs.188750 | ESTs | 1.56 | 3.28 |
| 418992 | AW074143 | Hs.87134 | ESTs | 1.88 | 2.20 |
| 409367 | AW382767 | | gb: PM0-HT0339-081199-001-h05 HT0339 Homo | 1.30 | 2.50 |
| 434973 | AW449285 | Hs.313636 | EST | 1.11 | 2.65 |
| 408383 | BE466959 | Hs.144153 | ESTs | 1.30 | 2.44 |
| 440100 | BE382685 | Hs.158549 | ESTs, Weakly similar to T2D3_HUMAN TRANS | 1.23 | 2.71 |
| 431996 | AL122087 | Hs.272304 | Homo sapiens mRNA; cDNA DKFZp564C0371 (f | 1.24 | 2.27 |
| 427681 | AB018263 | Hs.180338 | tumor necrosis factor receptor superfami | 1.70 | 1.68 |
| 405146 | | | C8001690*: gi\|6754446\|ref\|NP_034760.1\|ki | 2.00 | 0.68 |
| 436154 | AA764950 | Hs.119898 | ESTs | 1.43 | 3.00 |
| 451233 | AA047221 | Hs.59752 | ESTs | 1.38 | 2.20 |
| 446856 | AI814373 | Hs.164175 | ESTs | 1.33 | 3.93 |
| 448211 | BE384592 | Hs.6451 | PRO0659 protein | 1.48 | 2.73 |
| 418283 | S79895 | Hs.83942 | cathepsin K (pycnodysostosis) | 1.33 | 2.68 |
| 409609 | AW444670 | Hs.335685 | ESTs | 1.27 | 1.51 |
| 450414 | AI907735 | Hs.21446 | KIAA1716 protein | 1.60 | 1.24 |
| 452929 | AW954938 | Hs.172816 | neuregulin 1 | 2.01 | 3.70 |
| 435112 | AW976145 | Hs.143198 | inhibitor of growth family, member 3 | 1.22 | 1.30 |
| 439806 | AA846824 | Hs.180908 | ESTs | 0.80 | 2.04 |
| 439910 | H66765 | Hs.339397 | ESTs | 1.28 | 2.16 |
| 437886 | BE264111 | Hs.31314 | retinoblastoma-binding protein 7 | 1.06 | 2.82 |
| 441354 | AA931221 | Hs.126813 | ESTs | 1.20 | 2.28 |
| 428951 | AL138153 | Hs.300410 | ESTs, Moderately similar to A47582 B-cel | 1.50 | 1.83 |
| 438272 | AI167963 | Hs.143700 | ESTs, Weakly similar to S65824 reverse t | 1.34 | 2.51 |
| 429642 | X68264 | Hs.211579 | melanoma cell adhesion molecule | 1.18 | 1.18 |
| 422121 | AI767949 | Hs.179833 | ESTs | 1.18 | 2.26 |
| 411184 | AW821117 | | gb: PM2-ST0303-170100-003-g03 ST0303 Homo | 1.18 | 2.21 |
| 435871 | AF257077 | Hs.283627 | eukaryotic translation initiation factor | 1.17 | 1.57 |
| 430570 | AI417881 | Hs.292464 | ESTs | 1.49 | 3.17 |
| 431995 | AL080197 | Hs.272302 | hypothetical protein | 1.52 | 2.11 |
| 451326 | AW296946 | Hs.256078 | ESTs | 1.19 | 2.18 |
| 437046 | BE149154 | | gb: RC2-HT0252-271099-017-c11 HT0252 Homo | 1.18 | 2.25 |
| 410154 | F06959 | | gb: HSC1QD011 normalized infant brain cDN | 1.41 | 2.05 |
| 434373 | AI565566 | Hs.168587 | ESTs | 1.39 | 1.33 |
| 444552 | AW295211 | Hs.230777 | ESTs | 1.36 | 2.20 |
| 411608 | AW853441 | | gb: RC1-CT0252-030100-023-g09 CT0252 Homo | 2.12 | 1.80 |
| 440573 | BE550891 | Hs.270624 | ESTs | 2.19 | 2.17 |
| 443047 | AW157377 | Hs.132910 | ESTs | 1.81 | 2.28 |
| 451473 | AW298047 | Hs.346198 | ESTs | 1.18 | 2.30 |
| 416265 | AA177088 | Hs.190065 | ESTs | 2.37 | 3.38 |
| 435375 | AI733610 | Hs.187832 | ESTs | 1.12 | 2.18 |
| 401469 | | | NM_022137*: Homo sapiens secreted modular | 1.32 | 1.61 |
| 456152 | AA174126 | Hs.332163 | ESTs | 1.26 | 2.50 |
| 415808 | R21439 | Hs.334578 | Homo sapiens, clone IMAGE: 3929520, mRNA | 1.39 | 2.43 |
| 452721 | AJ269529 | Hs.301871 | solute carrier family 37 (glycerol-3-pho | 0.92 | 2.20 |
| 435127 | AI217926 | Hs.179863 | EST | 1.36 | 2.65 |
| 420772 | AW752656 | Hs.222707 | KIAA1718 protein | 1.19 | 1.50 |
| 456332 | AA228357 | | gb: nc39d05.r1 NCI_CGAP_Pr2 Homo sapiens | 1.45 | 3.57 |
| 444678 | AI741513 | Hs.143739 | ESTs | 1.43 | 1.62 |
| 446175 | AL036568 | Hs.291 | glutamyl aminopeptidase (aminopeptidase | 1.00 | 1.53 |
| 416463 | H59241 | | Homo sapiens cDNA FLJ11095 fis, clone PL | 1.44 | 2.13 |
| 405158 | | | ENSP00000243337: CDNA FLJ13984 fis, clone | 1.38 | 2.68 |
| 403903 | | | C5001632*: gi\|10645308\|gb\|AAG21430.1\|AC00 | 1.32 | 1.43 |
| 407271 | X98937 | | gb: H. sapiens rearranged lg heavy chain ( | 1.40 | 2.68 |
| 413929 | BE501689 | Hs.75617 | collagen, type IV, alpha 2 | 1.59 | 1.33 |
| 450778 | U81375 | Hs.25450 | solute carrier family 29 (nucleoside tra | 1.17 | 1.10 |
| 434274 | AA628539 | Hs.116252 | ESTs, Moderately similar to ALU1_HUMAN A | 1.92 | 2.80 |
| 400075 | | | Eos Control | 1.76 | 2.60 |
| 433694 | AI208611 | Hs.12066 | Homo sapiens cDNA FLJ11720 fis, clone HE | 1.48 | 2.33 |
| 454826 | AW833676 | | gb: QV4-TT0008-181199-038-h04 TT0008 Homo | 1.57 | 2.89 |
| 415168 | AA160805 | Hs.199832 | ESTs, Weakly similar to I78885 serine/th | 2.08 | 1.76 |
| 439486 | AF086303 | Hs.103185 | ESTs | 1.49 | 2.19 |
| 403291 | | | Target Exon | 1.36 | 2.28 |
| 438618 | AA897673 | Hs.123457 | ESTs | 0.75 | 0.79 |
| 455087 | AW855389 | | gb: CM3-CT0275-191099-024-e06 CT0275 Homo | 0.91 | 2.63 |
| 408075 | AA382881 | Hs.42409 | CGI-146 protein | 1.46 | 2.15 |
| 436826 | AA731863 | Hs.120276 | ESTs | 1.04 | 3.11 |
| 408961 | AW297475 | Hs.323180 | ESTs | 1.25 | 1.39 |
| 424408 | AI754813 | Hs.146428 | collagen, type V, alpha 1 | 1.64 | 2.05 |
| 423300 | AK000742 | Hs.126774 | L2DTL protein | 1.47 | 3.44 |
| 403217 | AL134878 | | ribosomal protein, large P2 | 1.70 | 2.22 |
| 437990 | AI686579 | Hs.121784 | ESTs | 2.14 | 1.69 |
| 419156 | AC002366 | Hs.46329 | amelogenin (X chromosome, amelogenesis i | 1.40 | 1.45 |
| 411817 | BE302900 | Hs.72241 | mitogen-activated protein kinase kinase | 1.18 | 1.12 |
| 425701 | AA361850 | Hs.322149 | Human clone 137308 mRNA, partial cds | 1.60 | 2.15 |
| 418757 | AI864193 | Hs.169728 | hypothetical protein FLJ13150 | 1.57 | 2.23 |
| 415184 | AA380436 | Hs.211973 | homolog of Yeast RRP4 (ribosomal RNA pro | 1.32 | 2.09 |
| 414918 | AI219207 | Hs.72222 | hypothetical protein FLJ13459 | 1.61 | 1.50 |
| 401723 | | | Target Exon | 1.01 | 2.68 |
| 439010 | AW170332 | Hs.75216 | Homo sapiens cDNA FLJ13713 fis, clone PL | 1.28 | 1.65 |
| 449166 | BE168981 | Hs.23131 | kinesin family member C3 | 1.64 | 2.58 |
| 410642 | AW792784 | | gb: CM0-UM0001-010300-258-h11 UM0001 Homo | 1.54 | 1.90 |
| 409556 | D38616 | Hs.54941 | phosphorylase kinase, alpha 2 (liver) | 1.63 | 1.21 |
| 439894 | AA853077 | Hs.300697 | immunoglobulin heavy constant gamma 3 (G | 0.76 | 0.61 |
| 401913 | | | ENSP00000249158*: CDNA | 0.97 | 2.59 |
| 406097 | | | Target Exon | 1.11 | 1.23 |
| 414745 | AA160511 | Hs.5326 | amino acid system N transporter 2; porcu | 1.29 | 1.12 |
| 445752 | AI733942 | Hs.344887 | ESTs | 2.03 | 1.68 |
| 408052 | AW501117 | Hs.283585 | ESTs | 1.32 | 1.72 |
| 407256 | AA204763 | Hs.288036 | tRNA isopentenylpyrophosphate transferas | 1.01 | 2.09 |
| 423264 | AJ133439 | Hs.126076 | Glutamate receptor interacting protein | 1.51 | 2.39 |
| 418859 | AA229558 | | gb: nc15d10.s1 NCI_CGAP_Pr1 Homo sapiens | 1.40 | 2.35 |
| 410370 | AB037753 | Hs.62767 | KIAA1332 protein | 1.34 | 2.00 |
| 417264 | AA195100 | Hs.188695 | ESTs | 1.09 | 2.61 |
| 444909 | AI933051 | Hs.192280 | ESTs | 1.56 | 2.92 |
| 419386 | AA236867 | | ESTs, Weakly similar to I38022 hypotheti | 1.14 | 2.28 |
| 439212 | AF087995 | Hs.134877 | ESTs | 1.06 | 2.90 |
| 437766 | W69171 | Hs.71741 | ESTs, Highly similar to I38945 melanoma | 1.25 | 2.28 |
| 448951 | AI611221 | Hs.334802 | hypothetical protein FLJ10101 | 1.88 | 2.17 |
| 401659 | | | Target Exon | 1.63 | 2.05 |
| 419145 | N99638 | | gb: za39g11.r1 Soares fetal liver spleen | 2.63 | 3.85 |
| 444813 | AW054834 | Hs.210356 | ESTs | 1.73 | 2.14 |
| 433902 | AW292820 | Hs.144906 | ESTs | 1.88 | 2.46 |
| 403072 | | | NM_003319*: Homo sapiens titin (TTN), mRN | 1.32 | 2.91 |
| 452484 | AB033042 | Hs.29679 | cofactor required for Sp1 transcriptiona | 0.72 | 0.70 |
| 456788 | AA724612 | Hs.133130 | Homo sapiens mRNA; cDNA DKFZp566H0124 (f | 1.90 | 3.40 |
| 403315 | | | Target Exon | 1.22 | 2.00 |
| 406432 | AJ289116 | | CD1E antigen, e polypeptide | 2.31 | 2.63 |
| 457785 | AA682670 | Hs.160884 | ESTs | 0.96 | 2.38 |
| 433259 | AA580665 | Hs.326082 | ESTs | 1.46 | 1.07 |
| 436882 | AW016722 | Hs.194976 | SH2 domain-containing phosphatase anchor | 1.38 | 2.13 |
| 401473 | | | Target Exon | 1.47 | 2.04 |
| 444816 | Z48633 | Hs.283742 | H. sapiens mRNA for retrotransposon | 1.64 | 2.15 |
| 438029 | H61502 | Hs.10235 | chromosome 5 open reading frame 4 | 1.44 | 2.25 |
| 431830 | Y16645 | Hs.271387 | small inducible cytokine subfamily A (Cy | 1.26 | 2.21 |
| 450817 | N71597 | Hs.29698 | ESTs, Weakly similar to ZN91_HUMAN ZINC | 2.20 | 2.90 |
| 404427 | | | C8000068*: gi\|5453579\|ref\|NP_006120.1\| bo | 0.74 | 0.81 |
| 430658 | AW970093 | Hs.24453 | ESTs | 1.45 | 2.55 |
| 405723 | | | Target Exon | 1.60 | 2.28 |
| 436896 | AW977385 | Hs.278615 | ESTs | 1.17 | 1.64 |
| 411974 | AW880414 | Hs.84264 | acidic protein rich in leucines | 1.54 | 2.08 |
| 412528 | AI123478 | Hs.32112 | ESTs | 1.72 | 2.85 |
| 446425 | AW295364 | Hs.255418 | ESTs | 1.25 | 1.31 |
| 424991 | AA775471 | Hs.241467 | ESTs | 0.62 | 0.37 |
| 443100 | AI033188 | | gb: ow94e08.s1 Soares_fetal_liver_spleen_{—} | 1.15 | 2.34 |
| 445332 | AI220225 | Hs.321057 | ESTs | 1.07 | 2.00 |
| 414781 | D50917 | Hs.77293 | KIAA0127 gene product | 1.04 | 3.43 |
| 421893 | NM_001078 | Hs.109225 | vascular cell adhesion molecule 1 | 1.15 | 2.53 |
| 424265 | AF173901 | Hs.144287 | hairy/enhancer-of-split related with YRP | 1.26 | 2.00 |
| 446667 | BE161878 | Hs.224805 | ESTs | 1.12 | 2.13 |
| 426399 | AA652588 | Hs.301348 | Homo sapiens cDNA FLJ13271 fis, clone OV | 2.26 | 1.29 |
| 438190 | AA780020 | Hs.136798 | ESTs, Moderately similar to KBF3_HUMAN N | 1.44 | 2.07 |
| 406972 | M32053 | | gb: Human H19 RNA gene, complete cds. | 1.38 | 2.11 |
| 417086 | AA194446 | | ESTs, Weakly similar to S55024 nebulin, | 2.12 | 3.30 |
| 446410 | AI361109 | Hs.151721 | ESTs, Weakly similar to I38022 hypotheti | 1.14 | 2.33 |
| 427674 | NM_003528 | Hs.2178 | H2B histone family, member Q | 1.24 | 1.00 |
| 422526 | AA311763 | Hs.131056 | ESTs | 1.29 | 2.04 |
| 439317 | AF086127 | Hs.50600 | ESTs, Weakly similar to T47156 hypotheti | 1.26 | 2.12 |
| 409126 | AA063426 | | gb: zf70c08.s1 Soares_pineal_gland_N3HPG | 1.28 | 2.20 |
| 412093 | BE242691 | Hs.14947 | ESTs | 0.87 | 2.58 |
| 420169 | AA256126 | Hs.16179 | hypothetical protein FLJ23467 | 1.38 | 2.07 |
| 426096 | D87436 | Hs.166318 | lipin 2 | 2.00 | 2.25 |
| 402551 | | | NM_005012*: Homo sapiens receptor tyrosin | 0.80 | 0.82 |
| 405760 | | | Target Exon | 1.44 | 2.85 |
| 402901 | | | NM_025206*: Homo sapiens hypothetical pro | 1.63 | 1.27 |
| 453982 | AW014252 | Hs.252837 | ESTs | 1.44 | 2.03 |
| 424244 | AV647184 | Hs.143601 | hypothetical protein hCLA-iso | 1.40 | 1.18 |
| 439984 | BE559514 | Hs.275425 | hypothetical protein | 1.30 | 2.15 |
| 457297 | AW968188 | | gb: EST380383 MAGE resequences, MAGJ Homo | 1.64 | 3.17 |
| 415054 | AI733907 | | gb: zo86h09.y5 Stratagene ovarian cancer | 1.00 | 2.20 |
| 426273 | AI174861 | Hs.190623 | ESTs | 1.19 | 1.16 |
| 405187 | | | NM_014272: Homo sapiens a disintegrin-lik | 1.31 | 1.35 |
| 413939 | AL047051 | Hs.199961 | ESTs, Weakly similar to ALU7_HUMAN ALU S | 2.44 | 1.88 |
| 427596 | AA449506 | Hs.270143 | extracellular glycoprotein EMILIN-2 prec | 2.06 | 1.50 |
| 408049 | AW076098 | Hs.345588 | desmoplakin (DPI, DPII) | 2.01 | 3.90 |
| 406002 | | | Target Exon | 1.73 | 2.08 |
| 408284 | AW248254 | Hs.44101 | protein kinase PKNbeta | 1.28 | 1.31 |
| 431377 | AW178807 | Hs.246182 | ESTs | 1.40 | 2.70 |
| 451456 | AW386183 | Hs.210305 | ESTs | 1.44 | 2.08 |
| 427530 | AA405093 | Hs.126519 | ESTs | 1.07 | 1.12 |
| 431957 | AK002104 | Hs.272246 | hypothetical protein FLJ11292 | 1.27 | 2.89 |
| 422283 | AW411307 | Hs.114311 | CDC45 (cell division cycle 45, S. cerevis | 1.74 | 1.28 |
| 419600 | AA448958 | Hs.91481 | NEU1 protein | 1.13 | 1.15 |
| 423314 | AI400661 | Hs.127811 | disintegrin metalloproteinase with throm | 1.44 | 2.53 |
| 451690 | AW451469 | Hs.209990 | ESTs | 1.41 | 2.49 |
| 454662 | AW812715 | | gb: RC4-ST018-271099-011-g01 ST0185 Homo | 1.35 | 2.86 |
| 454413 | AI653672 | Hs.40092 | PNAS-123 | 1.79 | 2.03 |
| 416861 | AW977206 | Hs.151858 | ESTs | 1.52 | 2.10 |
| 415908 | H08623 | Hs.22833 | ESTs | 1.37 | 2.13 |
| 438942 | AW875398 | Hs.6451 | PRO0659 protein | 1.80 | 1.55 |
| 407618 | AW054922 | Hs.53478 | Homo sapiens cDNA FLJ12366 fis, clone MA | 2.16 | 3.18 |
| 429177 | AA447527 | Hs.207429 | ESTs | 1.74 | 3.19 |
| 448357 | N20169 | Hs.108923 | RAB38, member RAS oncogene family | 0.77 | 0.73 |
| 422008 | AJ000534 | Hs.110708 | sarcoglycan, epsilon | 1.52 | 4.08 |
| 434461 | AA744046 | Hs.133350 | ESTs, Weakly similar to I78885 serine/th | 1.66 | 2.16 |
| 413489 | BE144228 | | gb: MR0-HT0165-140200-009-d04 HT0165 Homo | 1.28 | 2.23 |
| 405551 | | | Target Exon | 1.11 | 1.19 |
| 441183 | BE562910 | Hs.92679 | Homo sapiens clone CDABP0014 mRNA sequen | 1.20 | 1.20 |
| 456034 | AW450979 | | gb: UI-H-BI3-ala-a-12-0-UI.s1 NCI_CGAP_Su | 1.98 | 2.53 |
| 420611 | AA994635 | Hs.129929 | ESTs | 1.46 | 2.15 |
| 422061 | BE178434 | Hs.267995 | ESTs, Moderately similar to G02654 ribos | 1.42 | 2.30 |
| 437908 | AI082424 | | ESTs | 1.38 | 2.21 |
| 423052 | M28214 | Hs.123072 | RAB3B, member RAS oncogene family | 0.74 | 0.43 |
| 401927 | | | C17000914*: gi\|8394367\|ref\|NP_058549.1\| s | 2.26 | 2.14 |
| 432967 | AA572949 | Hs.207566 | ESTs | 1.52 | 2.28 |
| 439159 | AF087972 | Hs.120938 | ESTs | 2.03 | 2.08 |
| 415357 | H22757 | Hs.13471 | ESTs | 1.82 | 2.07 |
| 442327 | AA991745 | Hs.42522 | ESTs | 1.48 | 2.88 |
| 430186 | AB020696 | Hs.234791 | KIAA0889 protein | 1.46 | 2.23 |
| 426971 | AI809984 | Hs.243209 | ESTs, Weakly similar to NPA1_HUMAN NEURO | 1.06 | 2.13 |
| 422687 | AW068823 | Hs.119206 | insulin-like growth factor binding prote | 1.61 | 1.37 |
| 432954 | AI076345 | Hs.214199 | ESTs | 1.19 | 2.84 |
| 429040 | AL035542 | Hs.248169 | olfactory receptor, family 2, subfamily | 1.25 | 1.44 |
| 414169 | AA136169 | Hs.149335 | ESTs | 1.59 | 2.51 |
| 419882 | AA687313 | Hs.190043 | ESTs | 1.20 | 2.50 |
| 426900 | AW163564 | Hs.142375 | ESTs | 1.87 | 1.77 |
| 418773 | T39748 | Hs.325474 | Target CAT | 1.35 | 2.02 |
| 439776 | AL360140 | Hs.176005 | Homo sapiens mRNA full length insert cDN | 1.32 | 2.10 |
| 428712 | AW085131 | Hs.190452 | KIAA0365 gene product | 1.41 | 1.52 |
| 408839 | AW277084 | | gb: xp61h09.x1 NCI_CGAP_Ov39 Homo sapiens | 1.14 | 2.03 |
| 450492 | AW290961 | Hs.201815 | ESTs | 1.17 | 2.21 |
| 434654 | AI825942 | Hs.139366 | Homo sapiens clone L5 polyadenylated HER | 1.62 | 2.31 |
| 457567 | AW939074 | | gb: QV1-DT0069-010200-057-c12 DT0069 Homo | 1.80 | 3.73 |
| 452426 | AI904823 | Hs.31297 | duodenal cytochrome b | 2.15 | 1.84 |
| 418559 | AA225048 | Hs.104207 | ESTs | 1.84 | 2.33 |
| 439099 | AB037800 | Hs.6462 | protein kinase C and casein kinase subst | 1.07 | 1.15 |
| 451984 | R60571 | Hs.27406 | Homo sapiens mRNA; cDNA DKFZp566F1946 (f | 1.18 | 1.22 |
| 420789 | AI670057 | Hs.199882 | ESTs | 2.24 | 2.55 |
| 456396 | AA236863 | Hs.188894 | ESTs, Weakly similar to APXL_HUMAN APICA | 1.07 | 1.14 |
| 402948 | | | NM_025206: Homo sapiens hypothetical prot | 2.41 | 1.83 |
| 426405 | AW296631 | Hs.283403 | ESTs | 1.28 | 1.07 |
| 439732 | AW629604 | Hs.167641 | hypothetical protein from EUROIMAGE 1703 | 0.85 | 0.77 |
| 416784 | AA334592 | Hs.79914 | lumican | 1.88 | 1.27 |
| 422531 | AW967280 | Hs.293894 | ESTs, Weakly similar to HERC2 [H. sapiens | 1.20 | 1.25 |
| 415608 | F12795 | Hs.12286 | ESTs, Moderately similar to ALU1_HUMAN A | 1.03 | 2.31 |
| 428671 | BE297851 | Hs.189482 | zinc finger protein 179 | 1.26 | 2.20 |
| 420007 | H13700 | Hs.31235 | ESTs, Weakly similar to Y934_HUMAN HYPOT | 1.60 | 2.25 |
| 400850 | | | Target Exon | 1.22 | 1.03 |
| 404580 | | | trichorhinophalangeal syndrome I gene (T | 1.00 | 1.00 |
| 407680 | AW064284 | Hs.279153 | ESTs | 1.02 | 2.28 |
| 410420 | AA224053 | Hs.172405 | cell division cycle 27 | 1.64 | 1.00 |
| 421234 | AA907153 | Hs.190060 | ESTs | 1.76 | 1.45 |
| 426791 | AA384910 | Hs.46519 | ESTs | 1.12 | 2.15 |
| 430439 | AL133561 | | DKFZP434B061 protein | 1.00 | 1.00 |
| 434036 | AI659131 | Hs.197733 | hypothetical protein MGC2849 | 1.00 | 1.00 |
| 438915 | AA280174 | Hs.285681 | Williams-Beuren syndrome chromosome regi | 1.40 | 1.00 |
| 440304 | BE159984 | Hs.125395 | ESTs | 1.00 | 1.00 |
| 441699 | AW511126 | Hs.127572 | ESTs | 1.00 | 1.73 |
| 443383 | AI792453 | Hs.166507 | ESTs | 2.04 | 1.00 |
| 445660 | AI702668 | Hs.201955 | ESTs | 1.00 | 1.00 |
| 453160 | AI263307 | Hs.239884 | H2B histone family, member L | 1.00 | 1.00 |
| 456513 | AA279143 | Hs.88561 | ESTs | 1.00 | 1.40 |
| 457231 | AI472022 | Hs.301959 | proline synthetase co-transcribed (bacte | 0.98 | 0.63 |
| 459565 | W27086 | Hs.209694 | ESTs | 1.00 | 1.00 |
| 429317 | AA831552 | Hs.268016 | Homo sapiens cDNA: FLJ21243 fis, clone C | 1.00 | 1.00 |
| 430971 | M26150 | Hs.248177 | H3 histone family, member L | 1.14 | 1.40 |
| 408376 | AW971303 | Hs.292601 | ESTs | 1.08 | 1.60 |
| 411920 | AW876263 | | gb: PM4-PT0019-131299-006-E09 PT0019 Homo | 0.82 | 0.45 |
| 457389 | AW970989 | | gb: EST383074 MAGE resequences, MAGK Homo | 0.92 | 0.71 |
| 408565 | BE502544 | Hs.282244 | ESTs, Weakly similar to peptidoglycan re | 1.00 | 2.10 |
| 438086 | AA336519 | Hs.83623 | nuclear receptor subfamily 1, group I, m | 1.00 | 1.00 |
| 446779 | AI341135 | Hs.156084 | ESTs | 1.06 | 2.02 |
| 441691 | AI015418 | Hs.127556 | ESTs | 1.13 | 2.03 |
| 402039 | | | Target Exon | 0.44 | 0.36 |
| 437133 | AB018319 | Hs.5460 | KIAA0776 protein | 0.95 | 0.63 |
| 438089 | W05391 | | nuclear receptor subfamily 1, group I, m | 3.12 | 1.00 |
| 409582 | R27430 | Hs.271565 | ESTs | 1.00 | 1.00 |
| 428769 | AW207175 | Hs.106771 | ESTs | 1.00 | 1.48 |
| 442868 | AI022701 | Hs.336984 | ESTs | 1.27 | 1.62 |
| 439559 | AW364675 | Hs.173921 | ESTs, Weakly similar to 2109260A B cell | 1.00 | 1.33 |
| 426958 | R18845 | Hs.172979 | zinc finger protein 177 | 1.24 | 2.25 |
| 419015 | T79262 | Hs.14463 | ESTs | 1.16 | 2.03 |
| 415806 | AA169560 | | gb: zo89d08. r1 Stratagene ovarian cancer | 1.00 | 1.33 |
| 436110 | AA704899 | Hs.291651 | ESTs, Weakly similar to I38022 hypotheti | 1.60 | 2.21 |
| 458760 | AI498631 | Hs.111334 | ferritin, light polypeptide | 1.10 | 1.43 |
| 447342 | AI199268 | Hs.19322 | Homo sapiens, Similar to RIKEN cDNA 2010 | 1.28 | 1.00 |
| 438182 | AW342140 | Hs.182545 | ESTs, Weakly similar to ALU1_HUMAN ALU S | 0.90 | 2.31 |
| 438091 | AW373062 | | nuclear receptor subfamily 1, group I, m | 4.70 | 1.00 |
| 441633 | AW958544 | Hs.112242 | normal mucosa of esophagus specific 1 | 2.48 | 1.00 |
| 432222 | AI204995 | | gb: an03c03.x1 Stratagene schizo brain S1 | 1.96 | 2.84 |
| 416055 | Z45423 | Hs.13349 | Homo sapiens cDNA FLJ14647 fis, clone NT | 1.52 | 1.46 |
| 417895 | AA836392 | Hs.56237 | hypothetical protein FLJ20495 | 1.05 | 1.18 |
| 422959 | AV647015 | | paired immunoglobulin-like receptor beta | 1.33 | 1.25 |
| 408969 | AW297929 | Hs.328317 | EST | 1.88 | 2.07 |
| 409536 | H59024 | Hs.14485 | Homo sapiens cDNA: FLJ23220 fis, clone A | 1.18 | 2.38 |
| 447449 | AW137091 | Hs.18624 | KIAA1052 protein | 1.07 | 1.13 |
| 437315 | AW976247 | Hs.153248 | ESTs | 1.16 | 2.53 |
| 459317 | BRCA1b | | Eos Control | 1.36 | 1.32 |
| 405137 | | | Target Exon | 1.11 | 1.18 |
| 400366 | M22333 | | Target | 1.55 | 1.42 |
| 423413 | AA325560 | Hs.346401 | ESTs | 1.78 | 1.57 |
| 433972 | AI878910 | Hs.278670 | cisplatin resistance-associated overexpr | 1.62 | 2.98 |
| 440748 | AW451780 | Hs.130363 | ESTs | 1.42 | 2.14 |
| 422637 | AA399024 | Hs.118836 | myoglobin | 1.46 | 2.38 |
| 432342 | AL036128 | Hs.274404 | plasminogen activator, tissue | 1.67 | 1.10 |
| 442820 | AW293459 | Hs.172681 | ESTs | 1.02 | 1.13 |
| 436573 | AA723297 | Hs.127138 | ESTs | 1.18 | 1.15 |
| 403779 | | | Target Exon | 1.13 | 1.15 |
| 447686 | AI939440 | Hs.345192 | ESTs | 1.66 | 2.78 |
| 447506 | R78778 | Hs.29808 | Homo sapiens cDNA: FLJ21122 fis, clone C | 1.44 | 2.48 |
| 425853 | BE348404 | Hs.24740 | ESTs | 1.40 | 2.75 |
| 454457 | AW753456 | | gb: QV2-CT0261-261099-011-d11 CT0261 Homo | 1.52 | 2.00 |
| 424132 | AA335715 | Hs.200299 | ESTs | 1.34 | 1.32 |
| 421707 | NM_014921 | Hs.107054 | lectomedin-2 | 1.09 | 1.14 |
| 442871 | AI290691 | Hs.131393 | ESTs | 1.40 | 2.50 |
| 448489 | AI523875 | | gb: tg97d04.x1 NCI_CGAP_CLL1 Homo sapiens | 1.31 | 2.20 |
| 436365 | AW444548 | Hs.163118 | ESTs | 1.07 | 1.12 |
| 415733 | AI052628 | Hs.271570 | ESTs, Weakly similar to 2109260A B cell | 1.91 | 2.01 |
| 413888 | AA580288 | | gb: nn12d01.s1 NCI_CGAP_Co12 Homo sapiens | 1.75 | 1.93 |
| 408063 | BE086548 | Hs.42346 | calcineurin-binding protein calsarcin-1 | 1.92 | 1.73 |
| 442959 | AI025248 | Hs.6927 | ESTs | 1.05 | 1.12 |
| 409610 | AW444736 | Hs.27864 | ESTs | 1.62 | 2.45 |
| 424793 | AI559696 | Hs.298885 | ESTs | 1.37 | 2.78 |
| 449977 | C16939 | Hs.297848 | ESTs | 1.52 | 4.57 |
| 414051 | BE244127 | | gb: TCBAP1E0661 Pediatric pre-B cell acut | 1.68 | 2.84 |
| 422400 | AA974434 | Hs.128353 | ESTs | 1.04 | 2.20 |
| 443908 | AW295791 | Hs.13040 | G protein-coupled receptor 86 | 1.47 | 2.10 |
| 439316 | AF086126 | Hs.118208 | Homo sapiens cDNA FLJ11727 fis, clone HE | 2.08 | 0.59 |
| 438505 | AA808948 | Hs.173776 | ESTs, Moderately similar to ALU1_HUMAN A | 1.44 | 2.73 |
| 436196 | AK001084 | Hs.333498 | Homo sapiens cDNA FLJ10222 fis, clone HE | 2.17 | 1.00 |
| 453740 | AL120295 | Hs.311809 | ESTs, Moderately similar to PC4259 ferri | 1.88 | 2.58 |
| 431756 | R69465 | Hs.255889 | ESTs | 1.12 | 1.30 |
| 424487 | T08754 | Hs.6259 | KIAA1698 protein | 1.15 | 1.15 |
| 435392 | R07195 | Hs.19918 | ESTs | 1.38 | 2.64 |
| 430068 | AA464964 | | gb: zx80f10.s1 Soares ovary tumor NbHOT H | 0.92 | 2.12 |
| 418741 | H83265 | Hs.8881 | ESTs, Weakly similar to S41044 chromosom | 1.44 | 2.51 |
| 411664 | BE065069 | Hs.270833 | amphiregulin (schwannoma-derived growth | 1.94 | 2.33 |
| 416586 | D44643 | Hs.14144 | secreted modular calcium-binding protein | 1.72 | 2.68 |
| 419612 | AI498267 | Hs.110613 | KIAA0421 protein | 2.28 | 2.46 |
| 435800 | AI248285 | Hs.118348 | ESTs | 1.42 | 2.45 |
| 433363 | AA584829 | Hs.275163 | non-metastatic cells 2, protein (NM23B) | 2.07 | 2.53 |
| 422936 | AA319278 | | gb: EST21478 Adrenal gland tumor Homo sap | 1.46 | 1.22 |
| 413358 | BE259160 | Hs.75313 | aldo-keto reductase family 1, member B1 | 1.43 | 1.43 |
| 435357 | N71620 | Hs.118173 | ESTs | 1.44 | 2.93 |
| 441063 | AA913819 | Hs.188025 | ESTs | 1.20 | 2.80 |
| 450724 | R55428 | | gb: yj79b05.r1 Soares breast 2NbHBst Homo | 1.44 | 4.18 |
| 430446 | AF131782 | Hs.241438 | Homo sapiens clone 24941 mRNA sequence | 1.03 | 2.34 |
| 401577 | | | NM_000761: Homo sapiens cytochrome P450, | 1.13 | 1.22 |
| 403978 | | | C5000010*: gi\|10440464\|dbj\|BAB15765.1\| (A | 1.22 | 1.66 |
| 459702 | AI204995 | | gb: an03c03.x1 Stratagene schizo brain S1 | 2.72 | 4.60 |
| 416708 | H78836 | Hs.181900 | ESTs, Moderately similar to ALU1_HUMAN A | 1.36 | 2.03 |
| 451410 | AL110235 | Hs.26358 | DKFZP566K1924 protein | 1.51 | 2.28 |
| 451159 | AW298631 | Hs.27721 | Wolf-Hirschhorn syndrome candidate 1-lik | 1.33 | 1.09 |
| 448455 | AI252625 | Hs.269860 | ESTs, Moderately similar to S65657 alpha | 0.83 | 0.40 |
| 444020 | R92962 | Hs.35052 | ESTs | 1.66 | 2.50 |
| 414623 | BE391050 | | gb: 601285674F1 NIH_MGC_44 Homo sapiens c | 1.84 | 3.88 |
| 454915 | AW841619 | | gb: RC1-CN0017-120200-012-b09 CN0017 Homo | 1.14 | 2.10 |
| 444064 | W85970 | Hs.16292 | ESTs | 0.80 | 0.63 |
| 454353 | AW389693 | Hs.300700 | hypothetical protein FLJ20727 | 1.30 | 2.10 |
| 447794 | AI424999 | Hs.161445 | EST | 1.26 | 2.05 |
| 426686 | AI362802 | Hs.171814 | parathymosin | 1.16 | 1.11 |
| 435815 | AA700482 | Hs.113157 | ESTs | 1.66 | 2.73 |
| 432482 | L19267 | Hs.275924 | dystrophia myotonica-containing WD repea | 1.10 | 1.15 |
| 431062 | AA491270 | Hs.187946 | ESTs | 1.44 | 2.60 |
| 429191 | AF065215 | Hs.198161 | phospholipase A2, group IVB (cytosolic) | 1.35 | 1.06 |
| 424456 | AA341017 | Hs.25549 | hypothetical protein FLJ20898 | 1.21 | 3.45 |
| 451124 | AI186203 | Hs.31432 | cardiac ankyrin repeat protein | 1.23 | 2.10 |
| 432828 | AB042326 | Hs.287402 | chondroitin 4-sulfotransferase | 1.11 | 2.71 |
| 431868 | BE246400 | Hs.285176 | acetyl-Coenzyme A transporter | 1.03 | 3.10 |
| 429321 | AA449921 | | gb: zx37g07.r1 Soares _total_fetus_Nb2HF8_{—} | 1.68 | 2.93 |
| 417890 | R79048 | | gb: yi87g02.r1 Soares placenta Nb2HP Homo | 1.32 | 1.80 |
| 439590 | AF086410 | | gb: Homo sapiens full length insert cDNA | 1.32 | 2.43 |
| 420232 | AW450051 | Hs.256295 | ESTs | 1.28 | 2.26 |
| 418927 | BE349635 | Hs.190284 | ESTs | 1.46 | 1.23 |
| 441940 | AW298115 | Hs.128152 | ESTs | 1.34 | 1.34 |
| 401090 | | | C9000193*: gi\|6330729\|dbj\|BAA86547.1\| (AB | 1.50 | 1.40 |
| 409136 | AW206670 | Hs.50748 | chromosome 21 open reading frame 18 | 1.02 | 2.38 |
| 438267 | AW205708 | Hs.292725 | ESTs, Weakly similar to T18818 hypotheti | 1.28 | 2.25 |
| 422482 | AI439905 | Hs.344476 | gb: ti57g08.x1 NCI_CGAP_Lym12 Homo sapien | 1.05 | 2.81 |
| 420067 | T52431 | Hs.94795 | Homo sapiens mRNA; cDNA DKFZp564O222 (fr | 1.77 | 2.40 |
| 442180 | AA983913 | Hs.128929 | ESTs | 1.76 | 2.38 |
| 434256 | AI378817 | Hs.191847 | ESTs | 1.05 | 2.06 |
| 444519 | AI160304 | Hs.28313 | ESTs | 0.55 | 0.63 |
| 454459 | AW855738 | Hs.17767 | KIAA1554 protein | 1.10 | 2.05 |
| 455988 | BE177983 | | gb: RC3-HT0600-230300-021-g10 HT0600 Homo | 1.70 | 2.64 |
| 444510 | AI367823 | Hs.146872 | ESTs | 1.44 | 2.08 |
| 456210 | N49729 | Hs.156875 | ESTs | 1.64 | 2.65 |
| 450569 | AW192334 | Hs.38218 | ESTs | 1.78 | 2.71 |
| 414921 | BE390551 | Hs.77628 | steroidogenic acute regulatory protein r | 1.05 | 1.12 |
| 401381 | | | C14000165: gi\|12698069\|dbj\|BAB21853.1\| (A | 0.63 | 0.85 |
| 439998 | BE559554 | Hs.61790 | hypothetical protein FLJ23338 | 1.06 | 1.14 |
| 453762 | AW977286 | Hs.17428 | RBP1-like protein | 1.42 | 2.68 |
| 419403 | AA744520 | Hs.87734 | ESTs, Weakly similar to nonsyndromic hea | 0.99 | 2.17 |
| 423736 | AW936874 | | gb: RC1-DT0029-120100-011-f07 DT0029 Homo | 2.05 | 1.56 |
| 421186 | AI798039 | Hs.270563 | ESTs, Moderately similar to T12512 hypot | 1.29 | 1.31 |
| 426435 | AI827946 | Hs.124854 | hypothetical brain protein my040 | 1.36 | 1.59 |
| 439312 | AA833902 | Hs.270745 | ESTs | 1.60 | 2.50 |
| 407924 | BE537128 | Hs.299797 | ESTs | 1.51 | 1.15 |
| 409692 | AI500724 | | KIAA1550 protein | 1.72 | 2.21 |
| 415449 | H15034 | | gb: ym20a03.s1 Soares infant brain 1NIB H | 1.50 | 2.13 |
| 423436 | R21176 | Hs.100926 | ESTs | 1.18 | 2.60 |
| 458697 | AI797713 | Hs.156471 | ESTs | 1.54 | 2.20 |
| 415770 | M79237 | | gb: EST01385 Subtracted Hippocampus, Stra | 1.49 | 1.55 |
| 449279 | AI962312 | Hs.224976 | ESTs, Weakly similar to CRX_HUMAN CONE-R | 2.50 | 3.60 |
| 429735 | AA458759 | Hs.188794 | ESTs | 1.84 | 2.31 |
| 442124 | R66412 | Hs.129013 | Homo sapiens cDNA FLJ14309 fis, clone PL | 1.10 | 1.19 |
| 412557 | AA761612 | Hs.291557 | ESTs | 1.10 | 1.18 |
| 409335 | NM_001502 | Hs.53985 | glycoprotein 2 (zymogen granule membrane | 1.18 | 1.12 |
| 430526 | AF181862 | Hs.242407 | G protein-coupled receptor, family C, gr | 1.35 | 2.39 |
| 420855 | AA281092 | Hs.33417 | Homo sapiens cDNA: FLJ22806 fis, clone K | 1.22 | 2.09 |
| 450567 | AA033904 | Hs.269235 | ESTs | 1.60 | 2.20 |
| 414197 | W44877 | Hs.55501 | ESTs | 1.06 | 2.08 |
| 448800 | AI571294 | Hs.298889 | ESTs, Moderately similar to ALU1_HUMAN A | 1.65 | 2.79 |
| 421338 | AA287443 | | gb: zs52c10.r1 NCI_CGAP_GCB1 Homo sapiens | 1.47 | 2.90 |
| 412679 | BE144762 | | gb: CM0-HT0180-041099-065-b04 HT0180 Homo | 1.32 | 2.53 |
| 417882 | R22311 | | gb: yh26c09.r1 Soares placenta Nb2HP Homo | 1.58 | 2.43 |
| 425112 | AW953291 | Hs.64211 | hypothetical protein MGC5601 | 0.70 | 0.63 |
| 401658 | | | C16000210: gi\|12585542\|sp\|O14771\|Z213_HUM | 1.68 | 2.04 |
| 409325 | AW377549 | Hs.17865 | ESTs | 1.68 | 2.21 |
| 437402 | AI553976 | Hs.121191 | ESTs | 1.20 | 2.35 |
| 433455 | AA360439 | Hs.89319 | ESTs | 0.98 | 2.53 |
| 457329 | AI634860 | Hs.247043 | type 1 tumor necrosis factor receptor sh | 0.59 | 0.43 |
| 434830 | AW852235 | | gb: QV0-CT0225-230300-169-e11 CT0225 Homo | 1.24 | 1.12 |
| 450696 | AI654223 | Hs.16026 | hypothetical protein FLJ23191 | 1.44 | 2.53 |
| 446098 | AW072215 | Hs.208470 | ESTs | 1.38 | 2.93 |
| 443310 | BE552018 | Hs.133152 | ESTs | 0.85 | 0.83 |
| 424015 | N95696 | Hs.166361 | Homo sapiens mRNA; cDNA DKFZp564F112 (fr | 1.42 | 2.25 |
| 420229 | AA256675 | Hs.194058 | ESTs, Weakly similar to AF252293 1 PAR3 | 1.70 | 2.39 |
| 403371 | | | Target Exon | 1.50 | 3.43 |
| 410744 | H86002 | | gb: ys92b01.r1 Soares retina N2b5HR Homo | 1.32 | 2.13 |
| 424160 | T74062 | | gb: yc81f01.r1 Soares infant brain 1NIB H | 1.30 | 2.17 |
| 438818 | AW979008 | Hs.222487 | ESTs | 1.98 | 2.43 |
| 438791 | AA825750 | Hs.129983 | ESTs | 1.12 | 2.15 |
| 411206 | AW827390 | Hs.16899 | ESTs | 1.17 | 2.58 |
| 432211 | BE274530 | Hs.273333 | hypothetical protein FLJ10986 | 0.42 | 0.30 |
| 448918 | AB011152 | Hs.22572 | KIAA0580 protein | 1.54 | 2.63 |
| 424496 | AI733451 | Hs.167165 | hypothetical protein FLJ12975 | 1.39 | 2.25 |
| 410730 | AW368860 | | DnaJ (Hsp40) homolog, subfamily B, membe | 1.84 | 3.23 |
| 457581 | AA578512 | | gb: nh22e11.s1 NCI_CGAP_Pr1 Homo sapiens | 1.21 | 1.09 |
| 435353 | AW243062 | Hs.190348 | ESTs | 1.90 | 2.98 |
| 417029 | AW952192 | Hs.273385 | guanine nucleotide binding protein (G pr | 1.21 | 1.24 |
| 433682 | AA642418 | Hs.17381 | ESTs | 1.18 | 2.23 |
| 424915 | R42755 | Hs.23096 | ESTs | 1.60 | 2.73 |
| 442201 | AW516704 | Hs.208726 | ESTs | 1.74 | 3.20 |
| 429111 | AI870811 | Hs.7579 | KIAA1151 protein | 1.27 | 1.40 |
| 429282 | N27596 | Hs.21342 | ESTs | 1.84 | 3.73 |
| 436604 | AW105129 | Hs.242158 | ESTs | 1.27 | 2.70 |
| 448712 | W01046 | Hs.333371 | Homo sapiens clone TA40 untranslated mRN | 0.79 | 2.70 |
| 412274 | AA101443 | | gb: zn74a07.r1 Stratagene NT2 neuronal pr | 1.40 | 2.53 |
| 403859 | | | C5001408*: gi\|12621134\|ref\|NP_075244.1\|M | 1.76 | 2.00 |
| 451521 | AA018237 | Hs.128189 | gb: ze53a02.r1 Soares retina N2b4HR Homo | 1.48 | 2.51 |
| 443210 | AI692649 | Hs.9451 | hypothetical protein MGC13168 | 1.44 | 2.60 |
| 442722 | AL048889 | Hs.131029 | ESTs, Weakly similar to B28096 line-1 pr | 1.20 | 2.25 |
| 400840 | | | Target Exon | 0.66 | 0.60 |
| 454639 | AW811633 | | gb: RC2-ST0158-091099-011-d05 ST0158 Homo | 1.23 | 1.41 |
| 439864 | AI720078 | Hs.291997 | ESTs, Weakly similar to A47582 B-cell gr | 1.77 | 2.12 |
| 410725 | AW799279 | | gb: RC0-UM0051-210300-012-h06 UM0051 Homo | 1.08 | 2.55 |
| 423430 | AF112481 | Hs.128501 | RAD54, S. cerevisiae, homolog of, B | 1.94 | 2.29 |
| 450717 | T94709 | | gb: ye35d09.r1 Stratagene lung (937210) H | 1.56 | 2.64 |
| 400314 | NM_018949 | Hs.192720 | G protein-coupled receptor 14 | 0.89 | 0.87 |
| 434947 | AA654320 | Hs.183819 | Homo sapiens cDNA FLJ12304 fis, clone MA | 1.19 | 2.14 |
| 453582 | AW854339 | Hs.33476 | hypothetical protein FLJ11937 | 1.24 | 2.19 |
| 409005 | AW299806 | Hs.297256 | ESTs | 1.24 | 2.03 |
| 406584 | | | Target Exon | 1.52 | 2.37 |
| 420203 | AA256374 | Hs.191069 | ESTs | 1.16 | 1.37 |
| 406156 | | | Target Exon | 1.18 | 1.17 |
| 422132 | AB002337 | Hs.112078 | KIAA0339 gene product | 1.08 | 1.16 |
| 441371 | AW452292 | Hs.197354 | ESTs | 1.19 | 2.00 |
| 434807 | AA364183 | Hs.323443 | hypothetical protein FLJ11806 | 1.30 | 2.76 |
| 424542 | AI860558 | Hs.272009 | ESTs, Weakly similar to ALU2_HUMAN ALU S | 1.48 | 2.48 |
| 450893 | AK002185 | Hs.25625 | hypothetical protein FLJ11323 | 1.15 | 1.57 |
| 418481 | M81945 | Hs.85289 | CD34 antigen | 2.16 | 1.76 |
| 443077 | AI459490 | Hs.60090 | Homo sapiens cDNA FLJ13595 fis, clone PL | 1.36 | 2.41 |
| 437521 | AA758756 | Hs.121380 | ESTs | 1.07 | 2.05 |
| 430265 | L36033 | Hs.237356 | stromal cell-derived factor 1 | 2.34 | 1.35 |
| 446898 | AV660906 | Hs.184411 | albumin | 1.52 | 1.33 |
| 429725 | AA457367 | Hs.191638 | ESTs | 1.38 | 3.00 |
| 425114 | AW409763 | Hs.50699 | ESTs, Weakly similar to 2109260A B cell | 1.13 | 2.34 |
| 419879 | Z17805 | Hs.93564 | Homer, neuronal immediate early gene, 2 | 1.62 | 1.71 |
| 435284 | AA879470 | Hs.96849 | Homo sapiens cDNA FLJ11492 fis, clone HE | 1.20 | 2.50 |
| 415634 | F13165 | Hs.12549 | ESTs, Weakly similar to 2109260A B cell | 1.46 | 2.35 |
| 420565 | AI806770 | Hs.30258 | ESTs | 1.39 | 3.85 |
| 419494 | W01060 | Hs.34382 | ESTs | 1.10 | 1.75 |
| 458183 | AL031591 | Hs.7370 | phosphotidylinositol transfer protein, b | 1.28 | 2.24 |
| 416620 | R93080 | Hs.35035 | ESTs | 1.81 | 2.58 |
| 431356 | AW499632 | Hs.288512 | Homo sapiens cDNA FLJ11632 fis, clone HE | 1.42 | 2.03 |
| 433282 | BE539101 | Hs.5324 | hypothetical protein | 0.33 | 0.20 |
| 456898 | NM_001928 | Hs.155597 | D component of complement (adipsin) | 1.50 | 0.95 |
| 458126 | AW979136 | Hs.124629 | ESTs | 1.34 | 1.32 |
| 414005 | AA134489 | Hs.269379 | ESTs | 1.52 | 2.07 |
| 411496 | AW849241 | | gb: IL3-CT0215-210200-088-E03 CT0215 Homo | 1.10 | 2.21 |
| 451147 | AA016982 | Hs.64341 | ESTs | 1.53 | 2.29 |
| 450238 | T89693 | Hs.138777 | ESTs | 1.32 | 2.28 |
| 449284 | BE502240 | Hs.38592 | hypothetical protein FLJ23342 | 1.46 | 1.40 |
| 449479 | AI797619 | Hs.197659 | ESTs | 0.72 | 0.66 |
| 403066 | | | Target Exon | 1.32 | 1.19 |
| 410118 | AW590680 | Hs.110802 | von Willebrand factor | 1.72 | 2.54 |
| 437674 | AI749921 | Hs.205377 | ESTs | 1.38 | 2.21 |
| 431065 | AA491286 | Hs.128792 | ESTs | 1.30 | 2.08 |
| 416352 | H78006 | Hs.19553 | ESTs | 1.05 | 1.14 |
| 452565 | BE066552 | | gb: RC3-BT0333-300300-017-h08 BT0333 Homo | 1.46 | 2.53 |
| 418115 | AW005376 | Hs.173280 | ESTs | 1.20 | 0.98 |
| 422031 | R66895 | Hs.28788 | ESTs | 1.37 | 1.37 |
| 446269 | AW263155 | Hs.14559 | hypothetical protein FLJ10540 | 1.64 | 2.20 |
| 457683 | AI821877 | Hs.140002 | ESTs, Moderately similar to ALU7_HUMAN A | 1.03 | 2.35 |
| 435521 | W23814 | Hs.6361 | mitogen-activated protein kinase kinase | 0.73 | 0.59 |
| 438874 | H02780 | Hs.347520 | gb: yj41a11.r1 Soares placenta Nb2HP Homo | 1.56 | 2.73 |
| 441167 | AA921754 | Hs.211781 | ESTs | 1.74 | 2.12 |
| 455917 | BE156765 | | gb: RC1-HT0370-120100-012-c09 HT0370 Homo | 1.29 | 1.35 |
| 419058 | AW675039 | Hs.1227 | aminolevulinate, delta-, dehydratase | 2.04 | 1.83 |
| 408651 | BE266928 | Hs.17126 | hypothetical protein MGC15912 | 1.30 | 1.23 |
| 442737 | AB002319 | Hs.8663 | KIAA0321 protein | 0.85 | 0.79 |
| 407134 | T51588 | | gb: yb27e06.s1 Stratagene fetal spleen (9 | 1.23 | 0.97 |
| 447492 | AI381619 | Hs.20188 | ESTs | 1.26 | 2.28 |
| 437840 | AA884836 | Hs.292014 | ESTs | 2.05 | 2.29 |
| 412294 | AA689219 | Hs.117176 | poly(A)-binding protein, nuclear 1 | 1.47 | 3.55 |
| 419909 | AL136653 | Hs.93675 | decidual protein induced by progesterone | 1.10 | 2.18 |
| 432569 | AI131140 | Hs.152434 | ESTs | 1.34 | 1.83 |
| 412252 | AW903782 | | gb: CM4-NN1032-190400-527-g09 NN1032 Homo | 1.26 | 2.00 |
| 444298 | Z17870 | | gb: HSDHII020 Stratagene cDNA library Hum | 1.36 | 2.68 |
| 445261 | T79759 | Hs.250651 | ESTs, Weakly similar to I38022 hypotheti | 0.95 | 2.23 |
| 418315 | T06475 | Hs.124962 | Homo sapiens, clone IMAGE: 3510191, mRNA, | 0.82 | 0.82 |
| 440357 | AA379353 | Hs.20950 | phospholysine phosphohistidine inorganic | 0.83 | 0.68 |
| 440867 | AI417007 | Hs.166338 | ESTs | 1.45 | 1.50 |
| 410956 | AW938322 | | gb: PM1-DT0054-231299-002-c02 DT0054 Homo | 1.06 | 2.95 |
| 446574 | AI310135 | Hs.335933 | ESTs | 1.54 | 2.45 |
| 447912 | AW576549 | Hs.165728 | ESTs, Weakly similar to I38022 hypotheti | 1.22 | 2.07 |
| 457741 | BE044740 | | gb: hm55g10.x1 NCI_CGAP_RDF1 Homo sapiens | 1.89 | 2.08 |
| 433762 | AA732484 | Hs.169399 | ESTs | 1.24 | 2.58 |
| 418156 | W17056 | Hs.83623 | nuclear receptor subfamily 1, group I, m | 3.71 | 1.38 |
| 409282 | AW966480 | | gb: EST378554 MAGE resequences, MAGI Homo | 1.70 | 1.70 |
| 425169 | AW292500 | Hs.128514 | ESTs | 1.13 | 1.12 |
| 458497 | AI161428 | Hs.75916 | splicing factor 3b, subunit 2, 145 kD | 1.26 | 2.28 |
| 405673 | | | NM_022775: Homo sapiens hypothetical prot | 2.00 | 1.00 |
| 442691 | AW341438 | Hs.278036 | ESTs | 1.38 | 2.28 |
| 424316 | AA676403 | Hs.145078 | regulator of differentiation (in S. pomb | 1.06 | 2.10 |
| 444608 | AI174683 | Hs.329863 | ESTs | 1.95 | 1.82 |
| 447345 | BE247767 | Hs.18166 | KIAA0870 protein | 1.26 | 2.10 |
| 439848 | AW979249 | | gb: EST391359 MAGE resequences, MAGP Homo | 1.68 | 2.63 |
| 428946 | D42046 | Hs.194665 | DNA2 (DNA replication helicase, yeast, h | 1.32 | 2.33 |
| 403214 | | | NM_016232*: Homo sapiens interleukin 1 re | 1.02 | 2.15 |
| 404495 | | | C8001441*: gi\|8923061\|ref\|NP_060114.1\|hy | 2.20 | 2.49 |
| 443471 | AW236939 | Hs.172154 | Homo sapiens clone FLB3442 PRO0872 mRNA, | 1.58 | 1.74 |
| 437116 | AL049253 | Hs.190162 | ESTs | 1.22 | 2.53 |
| 451357 | AB020640 | Hs.26319 | Human DNA sequence from clone RP3-467L1 | 0.94 | 2.35 |
| 408255 | AW807321 | | gb: MR4-ST0062-240300-003-g05 ST0062 Homo | 1.12 | 1.39 |
| 448931 | AI597806 | Hs.192671 | ESTs | 1.30 | 3.29 |
| 422343 | AI628633 | Hs.346823 | gb: ty77d05.x1 NCI_CGAP_Kid11 Homo sapien | 1.86 | 2.32 |
| 407140 | AA059106 | Hs.271780 | ESTs, Weakly similar to I38022 hypotheti | 1.37 | 1.01 |
| 429187 | AA447648 | Hs.163872 | ESTs, Weakly similar to S65657 alpha-1C- | 1.74 | 1.55 |
| 423614 | AI457640 | Hs.206632 | ESTs | 1.48 | 2.83 |
| 429073 | AA446167 | Hs.47385 | ESTs | 1.24 | 2.00 |
| 415732 | AA167566 | Hs.271570 | ESTs, Weakly similar to 2109260A B cell | 1.31 | 2.34 |
| 412634 | U55984 | Hs.289088 | heat shock 90 kD protein 1, alpha | 0.42 | 0.22 |
| 415274 | AF001548 | Hs.78344 | myosin, heavy polypeptide 11, smooth mus | 1.94 | 1.27 |
| 415007 | BE244332 | Hs.77770 | adaptor-related protein complex 3, mu 2 | 0.78 | 0.71 |
| 402654 | | | Target Exon | 0.90 | 0.85 |
| 457974 | AW842353 | Hs.321717 | ESTs, Weakly similar to S22765 heterogen | 0.86 | 0.90 |
| 405340 | | | C2002952: gi\|1345964\|sp\|P10079\|FBP1_STRPU | 1.46 | 2.33 |
| 426259 | BE395776 | Hs.168640 | ankylosis, progressive (mouse) homolog | 1.63 | 2.75 |
| 442237 | AW905607 | Hs.24567 | ESTs, Weakly similar to KBF3_HUMAN NUCLE | 1.08 | 3.38 |
| 456370 | AA234938 | Hs.87384 | ESTs | 0.77 | 2.83 |
| 407041 | X15673 | | gb: Human pTR2 mRNA for repetitive sequen | 2.00 | 1.84 |
| 452001 | AI827675 | Hs.274281 | fidgetin | 1.38 | 2.03 |
| 445137 | AI733837 | Hs.145661 | ESTs | 1.60 | 3.00 |
| 440808 | AK001339 | Hs.7432 | hypothetical protein FLJ10477 | 1.17 | 2.10 |
| 404418 | | | Target Exon | 1.90 | 3.36 |
| 447658 | AI916872 | Hs.213424 | ESTs | 1.90 | 2.21 |
| 434414 | AI798376 | | gb: tr34b07.x1 NCI_CGAP_Ov23 Homo sapiens | 1.58 | 1.24 |
| 400834 | | | NM_002240*: Homo sapiens potassium inward | 1.25 | 2.33 |
| 449542 | AW857362 | Hs.268855 | ESTs, Weakly similar to I38022 hypotheti | 1.46 | 1.28 |
| 441043 | AA913422 | Hs.192104 | ESTs | 1.26 | 1.09 |
| 403391 | | | C3001164*: gi\|1730196\|sp\|P50573\|GAR3_RAT | 1.46 | 2.55 |
| 449129 | AI631602 | Hs.258949 | ESTs | 1.27 | 2.48 |
| 418321 | D63477 | Hs.84087 | KIAA0143 protein | 0.56 | 0.52 |
| 426789 | F06596 | Hs.23837 | Homo sapiens cDNA FLJ11812 fis, clone HE | 1.31 | 2.06 |
| 443679 | AK001810 | Hs.9670 | hypothetical protein FLJ10948 | 1.34 | 1.22 |
| 428554 | R46070 | Hs.6407 | ESTs | 1.04 | 2.08 |
| 401890 | | | Target Exon | 1.24 | 1.14 |
| 419501 | AW843822 | | gb: CM4-CN0045-010200-514-f08 CN0045 Homo | 1.74 | 1.38 |
| 457096 | AI809202 | Hs.208343 | ESTs, Weakly similar to cerebroside sulf | 0.82 | 0.87 |
| 426123 | AA370352 | | gb: EST82246 Prostate gland I Homo sapien | 1.28 | 2.35 |
| 449445 | AW197349 | Hs.232197 | ESTs | 1.24 | 2.13 |
| 430683 | AC004862 | Hs.247768 | Homo sapiens PAC clone RP4-697H17 from 7 | 1.30 | 2.00 |
| 440642 | AI744995 | | ESTs, Moderately similar to ALU4_HUMAN A | 1.29 | 2.44 |
| 455236 | AW875972 | | gb: CM3-PT0014-071299-051-b05 PT0014 Homo | 1.78 | 2.95 |
| 449622 | AW013915 | Hs.196578 | ESTs | 1.42 | 2.20 |
| 415116 | AA160363 | Hs.269956 | ESTs | 2.02 | 1.03 |
| 457269 | AI338993 | Hs.134535 | ESTs | 1.93 | 1.35 |
| 427877 | AW138725 | Hs.178067 | ESTs | 1.91 | 2.42 |
| 454631 | AW811324 | | gb: IL3-ST0141-131099-017-A02 ST0141 Homo | 1.00 | 3.13 |
| 458390 | AI792585 | Hs.133272 | ESTs, Weakly similar to ALUC_HUMAN !!!! | 1.02 | 2.21 |
| 435844 | AA700856 | Hs.59651 | ESTs, Weakly similar to 178885 serine/th | 0.85 | 0.81 |
| 427237 | AA399964 | Hs.97763 | ESTs | 1.57 | 1.44 |
| 408855 | T83061 | Hs.319946 | Homo sapiens mRNA for KIAA1727 protein, | 1.20 | 3.13 |
| 442151 | AI733404 | Hs.128865 | ESTs | 1.50 | 2.13 |
| 412708 | R26830 | Hs.106137 | ESTs, Weakly similar to CGHU7L collagen | 1.16 | 3.00 |
| 417262 | AA195276 | Hs.263858 | ESTs, Moderately similar to B34087 hypot | 1.25 | 2.40 |
| 419362 | N64116 | Hs.24624 | hypothetical protein FLJ21945 | 1.38 | 2.48 |
| 447248 | AW295831 | Hs.6496 | ESTs | 1.56 | 2.03 |
| 415622 | F13010 | Hs.12400 | ESTs | 1.48 | 2.30 |
| 414065 | AW515373 | Hs.271249 | Homo sapiens cDNA FLJ13580 fis, clone PL | 1.26 | 2.88 |
| 414585 | W46954 | Hs.334716 | hypothetical protein MGC16291 | 1.24 | 2.05 |
| 443197 | Z43613 | | gb: HSC1GD091 normalized infant brain cDN | 1.11 | 2.04 |
| 428266 | AI382001 | Hs.43590 | ESTs | 1.09 | 2.03 |
| 447083 | AI472124 | Hs.157757 | ESTs | 1.66 | 3.53 |
| 412302 | AW936334 | | gb: QV4-DT0021-281299-070-g05 DT0021 Homo | 1.74 | 3.00 |
| 445555 | AW974013 | | ESTs | 1.32 | 1.29 |
| 453117 | AW162044 | Hs.104203 | hypothetical protein MGC12981 | 0.73 | 0.81 |
| 436757 | AW975663 | Hs.293404 | ESTs, Weakly similar to ALU1_HUMAN ALU S | 2.21 | 1.88 |
| 431976 | AA719001 | Hs.291065 | ESTs | 1.23 | 2.01 |
| 430657 | AA482910 | Hs.279664 | ESTs | 1.64 | 2.65 |
| 438744 | BE314727 | Hs.75721 | profilin 1 | 0.85 | 0.85 |
| 439325 | AF086139 | Hs.150423 | cyclin-dependent kinase 9 (CDC2-related | 1.16 | 2.05 |
| 438117 | AA328041 | Hs.194329 | hypothetical protein FLJ21174 | 0.79 | 0.76 |
| 401686 | | | NM_014587*: Homo sapiens SRY (sex determi | 1.32 | 2.31 |
| 420269 | U72937 | Hs.96264 | alpha thalassemia/mental retardation syn | 0.78 | 0.53 |
| 434288 | AW189075 | Hs.116265 | fibrillin3 | 2.42 | 4.23 |
| 433215 | AB040912 | Hs.191098 | hypothetical protein FLJ11598 | 1.36 | 1.36 |
| 413429 | BE139117 | Hs.278881 | ESTs | 1.30 | 2.98 |
| 426417 | AA377908 | Hs.13254 | ESTs | 1.36 | 1.77 |
| 413882 | AA132973 | Hs.184492 | ESTs | 1.55 | 2.10 |
| 413346 | AA128586 | | gb: zI24h06.r1 Soares_pregnant_uterus_NbH | 1.29 | 1.77 |
| 445020 | AI205655 | Hs.147221 | ESTs | 1.90 | 2.00 |
| 418175 | AW967054 | Hs.206312 | ESTs, Weakly similar to I38022 hypotheti | 1.60 | 3.70 |
| 429582 | AI569068 | Hs.22247 | ESTs | 1.06 | 2.38 |
| 409134 | AW340389 | Hs.250585 | ESTs | 1.64 | 2.57 |
| 415642 | U19878 | Hs.336224 | transmembrane protein with EGF-like and | 0.73 | 2.33 |
| 435667 | F13625 | Hs.124183 | ESTs | 1.10 | 2.33 |
| 440513 | BE407106 | Hs.65907 | Homo sapiens, clone IMAGE: 3959816, mRNA, | 0.85 | 2.03 |
| 419711 | C02621 | Hs.159282 | ESTs | 1.22 | 2.00 |
| 434249 | AA987537 | Hs.129875 | ESTs | 1.29 | 2.48 |
| 437355 | AL359557 | Hs.306508 | Homo sapiens mRNA; cDNA DKFZp762O1415 (f | 1.86 | 2.08 |
| 428360 | H10291 | Hs.30974 | ESTs | 1.40 | 2.05 |
| 435339 | AI358300 | Hs.129827 | ESTs | 1.38 | 2.18 |
| 435345 | AW360966 | Hs.6653 | ESTs | 1.49 | 2.27 |
| 435105 | AI878982 | Hs.131859 | Homo sapiens F-box protein FBX11 mRNA, p | 1.78 | 2.33 |
| 459645 | AA074346 | Hs.250715 | ESTs | 1.50 | 2.40 |
| 449691 | AA002143 | Hs.21413 | solute carrier family 12, (potassium-chl | 0.74 | 0.69 |
| 425955 | T96509 | Hs.248549 | ESTs, Moderately similar to S65657 alpha | 1.42 | 2.76 |
| 437272 | AW975957 | | gb: EST388066 MAGE resequences, MAGN Homo | 1.00 | 2.16 |
| 456955 | NM_006925 | Hs.166975 | splicing factor, arginine/serine-rich 5 | 0.84 | 0.81 |
| 421362 | AK000050 | Hs.103853 | hypothetical protein FLJ20043 | 1.30 | 2.21 |
| 457926 | AA452378 | Hs.11637 | Homo sapiens mRNA; cDNA DKFZp547J125 (fr | 1.27 | 2.12 |
| 444557 | AI167637 | Hs.146924 | ESTs | 1.83 | 2.35 |
| 434476 | AW858520 | Hs.84264 | acidic protein rich in leucines | 1.43 | 3.80 |
| 458059 | AW015588 | Hs.137232 | ESTs, Weakly similar to S65657 alpha-1C- | 1.30 | 2.23 |
| 413595 | AW235215 | Hs.16145 | ESTs | 2.10 | 2.43 |
| 417281 | R98773 | Hs.268883 | ESTs | 1.26 | 2.10 |
| 445689 | BE158869 | | gb: QV0-HT0398-210100-096-f08 HT0398 Homo | 1.15 | 2.15 |
| 423249 | AA323682 | Hs.125374 | ESTs, Weakly similar to S26689 hypotheti | 1.76 | 1.50 |
| 408366 | AW511255 | Hs.346442 | ESTs | 1.74 | 2.91 |
| 441359 | AI435179 | Hs.126820 | ESTs | 2.43 | 1.59 |
| 413068 | BE063792 | | gb: QV3-BT0295-260100-066-d06 BT0295 Homo | 1.52 | 2.09 |
| 441322 | AW071851 | Hs.130628 | ESTs | 1.42 | 2.10 |
| 409124 | AW292809 | Hs.50727 | N-acetylglucosaminidase, alpha- (Sanfili | 1.11 | 2.20 |
| 432413 | AK000257 | Hs.274505 | Homo sapiens mRNA; cDNA DKFZp564A216 (fr | 1.10 | 2.25 |
| 425391 | AI248252 | Hs.160672 | ESTs | 1.17 | 2.38 |
| 443861 | AW449462 | Hs.134743 | ESTs | 1.44 | 2.30 |
| 454609 | AW810204 | | gb: MR4-ST0125-021199-017-d08 ST0125 Homo | 2.30 | 1.33 |
| 425893 | AA629695 | | gb: ad43b07.s1 Stratagene lung carcinoma | 1.76 | 2.51 |
| 443611 | NM_014397 | Hs.9625 | NIMA (never in mitosis gene a)-related k | 1.81 | 2.90 |
| 410359 | R38624 | Hs.106313 | ESTs | 1.78 | 2.05 |
| 406308 | | | NM_025192: Homo sapiens hypothetical prot | 1.92 | 2.24 |
| 432476 | T94344 | Hs.326263 | ESTs | 1.40 | 2.45 |
| 435073 | AA664078 | | gb: ac04a05.s1 Stratagene lung (937210) H | 1.66 | 2.26 |
| 420581 | AA278459 | Hs.151940 | ESTs | 1.48 | 2.58 |
| 435579 | AI332373 | Hs.156924 | ESTs | 1.46 | 2.68 |
| 439633 | AF086464 | Hs.86248 | ESTs | 1.40 | 2.48 |
| 430551 | AA481150 | Hs.136343 | ESTs | 1.40 | 2.28 |
| 450855 | T97988 | Hs.295605 | mannosidase, alpha, class 2A, member 2 | 1.48 | 2.40 |
| 444326 | AI939357 | Hs.270710 | ESTs | 0.88 | 2.28 |
| 412149 | R49355 | Hs.273824 | ESTs | 1.58 | 2.19 |
| 455116 | AW857271 | | gb: CM0-CT0307-210100-158-g09 CT0307 Homo | 1.56 | 2.50 |
| 449626 | AA774247 | Hs.301637 | zinc finger protein 258 | 0.60 | 0.53 |
| 410047 | AI167810 | Hs.132390 | zinc finger protein 36 (KOX 18) | 0.66 | 0.58 |
| 418865 | AW117500 | Hs.104241 | ESTs | 1.58 | 2.63 |
| 402762 | | | ENSP00000235171*: GAP junction beta-4 pro | 0.81 | 0.82 |
| 436449 | AI418027 | Hs.120361 | ESTs | 1.46 | 1.46 |
| 403488 | | | ENSP00000201948: KARYOPHERIN BETA2B HOMOL | 1.38 | 2.23 |
| 431235 | AA318271 | Hs.250905 | hypothetical protein | 1.14 | 2.55 |
| 448576 | AB026730 | Hs.21495 | UDP-Gal: betaGlcNAc beta 1,3-galactosyltr | 0.70 | 0.78 |
| 408100 | AW205382 | Hs.42676 | KIAA0781 protein | 1.36 | 2.66 |
| 433436 | AW162474 | | Bruno (Drosophila)-like 6, RNA binding | 1.50 | 2.15 |
| 422337 | R38572 | | gb: yc87c11.s1 Soares infant brain 1NIB H | 2.23 | 1.71 |
| 426160 | AA206020 | Hs.167460 | splicing factor, arginine/serine-rich 3 | 1.08 | 2.09 |
| 447008 | BE010189 | | nuclear receptor subfamily 1, group I, m | 1.26 | 1.27 |
| 420141 | AA702961 | Hs.124103 | ESTs, Weakly similar to I38344 titin, ca | 1.46 | 2.60 |
| 423840 | AA332434 | Hs.72465 | ESTs, Weakly similar to non-lens beta ga | 1.26 | 2.47 |
| 447793 | AI424924 | Hs.211203 | ESTs | 2.38 | 1.83 |
| 407328 | AA508857 | Hs.187748 | ESTs, Weakly similar to ALU1_HUMAN ALU S | 1.11 | 2.54 |
| 432451 | AW972771 | Hs.292471 | ESTs, Weakly similar to ALU1_HUMAN ALU S | 1.63 | 2.05 |
| 421311 | N71848 | Hs.283609 | hypothetical protein PRO2032 | 0.51 | 0.44 |
| 444649 | AW207523 | Hs.197628 | ESTs | 1.21 | 2.24 |
| 448688 | R94570 | Hs.266869 | ESTs, Weakly similar to ALU1_HUMAN ALU S | 1.91 | 3.25 |
| 428847 | AI954833 | Hs.98881 | ESTs | 1.48 | 2.66 |
| 413750 | BE161453 | | gb: IL2-HT0437-290200-045-A06 HT0437 Homo | 1.22 | 1.00 |
| 429355 | AW973253 | Hs.292689 | ESTs | 1.86 | 2.35 |
| 427798 | AA412499 | Hs.104779 | ESTs | 1.82 | 2.33 |
| 431179 | AI338644 | Hs.195432 | aldehyde dehydrogenase 2 family (mitocho | 0.80 | 2.00 |
| 451719 | AI373532 | Hs.157910 | ESTs | 1.29 | 3.85 |
| 438094 | AI821755 | Hs.131805 | ESTs, Weakly similar to A56194 thromboxa | 1.74 | 2.54 |
| 418504 | BE159718 | Hs.85335 | Homo sapiens mRNA; cDNA DKFZp564D1462 (f | 0.52 | 0.49 |
| 467414 | AF072164 | | gb: Homo sapiens HSFE-1 mRNA, partial cds | 1.67 | 2.28 |
| 416410 | H53777 | Hs.36822 | ESTs | 1.85 | 2.28 |
| 439141 | AI241470 | Hs.268982 | ESTs | 1.08 | 2.28 |
| 441181 | AA416925 | Hs.121076 | peptidylprolyl isomerase (cyclophilin)-l | 1.81 | 2.02 |
| 434482 | AF143331 | Hs.16073 | ESTs | 1.22 | 2.00 |
| 455757 | BE079531 | | gb: RC5-BT0624-240300-013-D08 BT0624 Homo | 1.53 | 2.16 |
| 425787 | AA363867 | Hs.155029 | ESTs | 0.76 | 2.13 |
| 405727 | | | CX001244: gi\|11420428\|ref\|XP_004814.1\|be | 1.70 | 2.21 |
| 441846 | AW850980 | | gb: IL3-CT0220-150200-068-B03 CT0220 Homo | 1.16 | 2.14 |
| 451945 | BE504055 | Hs.211420 | ESTs | 0.84 | 2.73 |
| 438432 | AW444990 | Hs.258800 | ESTs, Weakly similar to I38022 hypotheti | 1.60 | 2.43 |
| 451140 | AW411354 | Hs.26002 | LIM domain binding 1 | 1.14 | 1.20 |
| 407341 | AA918886 | Hs.204918 | ESTs, Weakly similar to ALU8_HUMAN ALU S | 1.03 | 2.42 |
| 453041 | AI680737 | Hs.289068 | Homo sapiens cDNA FLJ11918 fis, clone HE | 1.69 | 3.43 |
| 437613 | R19892 | Hs.10267 | MIL1 protein | 1.16 | 2.11 |
| 451507 | AW291109 | Hs.208787 | ESTs, Weakly similar to T31611 hypotheti | 1.22 | 2.05 |
| 430259 | BE550182 | Hs.127826 | RaIGEF-like protein 3, mouse homolog | 2.85 | 1.00 |
| 453669 | AL049029 | Hs.7258 | hypothetical protein FLJ22021 | 0.75 | 0.64 |
| 455065 | AW854352 | | gb: RC3-CT0255-200100-024-g10 CT0255 Homo | 1.49 | 2.20 |
| 442220 | AL037800 | Hs.8148 | selenoprotein T | 0.50 | 0.18 |
| 437936 | AW798475 | Hs.288549 | hypothetical protein FLJ14710 | 1.50 | 2.44 |
| 442556 | AL137761 | Hs.8379 | Homo sapiens mRNA; cDNA DKFZp586L2424 (f | 0.54 | 0.37 |
| 405223 | | | Target Exon | 1.09 | 2.80 |
| 437225 | AW975982 | Hs.292935 | ESTs | 1.03 | 2.47 |
| 421101 | AF010446 | Hs.101840 | major histocompatibility complex, class | 0.72 | 0.57 |
| 436200 | R51386 | Hs.124881 | ESTs | 1.64 | 2.93 |
| 402025 | | | NM_021624: Homo sapiens histamine H4 rece | 1.52 | 2.28 |
| 407019 | U49973 | | gb: Human Tigger1 transposable element, c | 2.40 | 2.12 |
| 451305 | AW003571 | Hs.211191 | ESTs, Weakly similar to A46010 X-linked | 1.24 | 3.23 |
| 423450 | AJ290445 | Hs.128759 | KIAA0524 protein | 1.64 | 2.13 |
| 423139 | AW402725 | Hs.288560 | hypothetical protein FLJ21106 | 1.61 | 2.28 |
| 451763 | AW294647 | Hs.233634 | hypothetical protein FLJ14220 | 1.39 | 2.08 |
| 458915 | AI915689 | Hs.212781 | EST | 1.62 | 2.02 |
| 452829 | AI955579 | Hs.63368 | ESTs, Weakly similar to TRHY_HUMAN TRICH | 0.60 | 0.41 |
| 446383 | T05816 | Hs.92511 | ESTs | 2.08 | 1.48 |
| 432576 | AW157424 | Hs.165954 | ESTs, Weakly similar to I38022 hypotheti | 1.88 | 2.49 |
| 433820 | AI401627 | Hs.174067 | ESTs | 1.30 | 2.00 |
| 419719 | AA844700 | Hs.39297 | ESTs, Moderately similar to ALU1_HUMAN A | 1.33 | 2.00 |
| 415868 | H06728 | Hs.21017 | ESTs | 1.34 | 2.08 |
| 420738 | NM_004185 | Hs.258575 | wingless-type MMTV integration site fami | 1.42 | 2.29 |
| 446614 | AK001733 | Hs.15562 | hypothetical protein FLJ10871 | 0.79 | 0.78 |
| 404167 | | | NM_021956*: Homo sapiens glutamate recept | 1.62 | 2.55 |
| 417074 | Z49878 | Hs.81131 | guanidinoacetate N-methyltransferase | 0.72 | 0.75 |
| 401215 | | | C12000457*: gi\|7512178\|pir∥T30337 polypr | 1.14 | 2.08 |
| 421600 | AW893889 | Hs.323231 | Homo sapiens cDNA FLJ11946 fis, clone HE | 1.88 | 2.66 |
| 426248 | T18988 | Hs.293668 | ESTs | 1.17 | 3.44 |
| 454523 | AW803980 | | gb: PM0-UM0084-240300-001-G11 UM0084 Homo | 1.34 | 2.40 |
| 420656 | AA279098 | Hs.187636 | ESTs | 1.22 | 2.43 |
| 402833 | | | C1002508: gi\|6691937\|emb\|CAB65797.1\|(AL0 | 1.31 | 2.00 |
| 438910 | AA827921 | Hs.291858 | ESTs, Weakly similar to ALUC_HUMAN !!!! | 1.39 | 3.13 |
| 416170 | H42454 | Hs.220645 | ESTs | 0.99 | 2.18 |
| 433598 | AI762836 | Hs.271433 | ESTs, Moderately similar to ALU2_HUMAN A | 2.04 | 1.28 |
| 417699 | T91491 | Hs.119670 | ESTs | 1.36 | 2.50 |
| 459605 | AL045773 | | gb: DKFZp434F246_r1 434 (synonym: htes3) | 1.21 | 2.13 |
| 453204 | R10799 | Hs.191990 | ESTs | 3.12 | 2.98 |
| 458971 | AL119206 | Hs.126257 | ESTs, Weakly similar to ALU1_HUMAN ALU S | 1.34 | 2.09 |
| 457040 | N77624 | Hs.173717 | phosphatidic acid phosphatase type 2B | 1.68 | 2.00 |
| 400414 | AF083118 | Hs.283968 | Homo sapiens CATX-2 mRNA, complete cds | 1.70 | 2.54 |
| 426263 | AI908774 | Hs.259785 | carnitine palmitoyltransferase I, liver | 0.96 | 2.14 |
| 439334 | AI148976 | Hs.112062 | ESTs | 1.50 | 2.45 |
| 455527 | AW984479 | | gb: PM1-HN0012-220300-001-b12 HN0012 Homo | 1.46 | 2.28 |
| 408084 | AL040832 | Hs.160422 | Homo sapiens clone PP902 unknown mRNA | 1.61 | 2.23 |
| 432059 | AF227131 | Hs.272387 | taste receptor, type 2, member 4 | 1.66 | 2.15 |
| 429791 | AW015667 | Hs.119427 | ESTs | 1.51 | 2.83 |
| 438695 | AI885190 | Hs.156089 | ESTs, Weakly similar to repressor protei | 1.19 | 2.03 |
| 458139 | AI525711 | Hs.253147 | ESTs | 1.42 | 2.10 |
| 413035 | BE155563 | | gb: PM4-HT0352-171199-001-C05 HT0352 Homo | 1.62 | 2.30 |
| 422444 | AA310688 | | gb: EST181501 Jurkat T-cells V Homo sapie | 1.38 | 2.05 |
| 409546 | AW410190 | Hs.250624 | hypothetical protein MGC4473 | 1.87 | 2.18 |
| 411432 | AW846272 | | gb: QV0-CT0179-300999-024-d12 CT0179 Homo | 1.04 | 2.03 |
| 445327 | AI220082 | Hs.147722 | ESTs | 1.16 | 2.10 |
| 424628 | AB011136 | Hs.151385 | KlAA0564 protein | 0.61 | 0.63 |
| 440197 | AW340708 | Hs.317714 | pallid (mouse) homolog, pallidin | 0.56 | 0.39 |
| 409894 | BE081731 | | gb: QV2-BT0635-220400-158-e04 BT0635 Homo | 1.50 | 2.45 |
| 422776 | AA316987 | Hs.129846 | ESTs | 1.36 | 2.20 |
| 428255 | AI627478 | Hs.187670 | ESTs | 1.34 | 2.40 |
| 412484 | AA112090 | Hs.269961 | ESTs | 0.97 | 2.00 |
| 432789 | D26361 | Hs.3104 | KIAA0042 gene product | 1.44 | 2.73 |
| 430100 | AA766178 | Hs.291601 | ESTs, Highly similar to T00350 hypotheti | 1.06 | 2.02 |
| 419528 | AA244000 | Hs.222365 | ESTs | 1.34 | 2.06 |
| 441793 | AA968459 | Hs.158785 | ESTs | 1.80 | 2.70 |
| 429468 | AF033579 | | T-box 10 | 0.71 | 0.61 |
| 410248 | AA166653 | Hs.268171 | ESTs | 2.55 | 2.10 |
| 401818 | | | NM_000664*: Homo sapiens acetyl-Coenzyme | 1.76 | 2.58 |
| 451724 | AI903765 | | gb: UI-BT037-301298-102 BT037 Homo sapien | 1.64 | 2.28 |
| 431866 | NM_012098 | Hs.8025 | angiopoietin-like 2 | 1.56 | 2.36 |
| 432719 | AW935411 | Hs.314460 | ESTs | 1.36 | 2.25 |
| 418977 | AA233094 | Hs.191517 | ESTs | 2.06 | 3.60 |
| 404220 | | | C6000989*: gi\|7573285\|emb\|CAB87644.1\| (AL | 1.54 | 2.23 |
| 446708 | BE549905 | Hs.231754 | ESTs | 1.35 | 2.16 |
| 453823 | AL137967 | | gb: DKFZp761D2315_r1 761 (synonym: hamy2) | 1.42 | 2.38 |
| 422050 | AA302741 | Hs.25786 | ESTs, Moderately similar to JC5238 galac | 1.40 | 2.50 |
| 400704 | | | Target Exon | 1.48 | 1.00 |
| 406104 | | | Target Exon | 1.22 | 2.03 |
| 411008 | AW813238 | | gb: MR3-ST0191-020200-207-d04 ST0191 Homo | 1.00 | 2.13 |
| 426582 | AA381797 | Hs.281121 | ESTs | 1.35 | 2.45 |
| 430853 | AI734179 | Hs.105676 | ESTs | 1.43 | 2.23 |
| 432420 | AL044659 | Hs.43791 | ESTs | 1.15 | 2.03 |
| 403197 | | | C2002793*: gi\|1353148\|sp\|Q09568\|YR86_CAEE | 0.52 | 0.47 |
| 432407 | AA221036 | | gb: zr03f12.r1 Stratagene NT2 neuronal pr | 1.93 | 2.23 |
| 414996 | AW747800 | Hs.55016 | hypothetical protein FLJ21935 | 1.56 | 2.72 |
| 401016 | | | ENSP00000227126: NAALADASE II PROTEIN. | 1.25 | 2.45 |
| 433335 | AA584134 | Hs.269454 | ESTs | 1.31 | 2.24 |
| 459668 | BE244127 | | gb: TCBAP1E0661 Pediatric pro-B cell acut | 1.16 | 2.03 |
| 437722 | AW292947 | Hs.122872 | ESTs, Weakly similar to JU0033 hypotheti | 3.75 | 2.72 |
| 452277 | AL049013 | Hs.28783 | KIAA1223 protein | 0.33 | 0.26 |
| 425712 | AA412548 | Hs.21423 | ESTs, Moderately similar to ALU1_HUMAN A | 1.34 | 2.21 |
| 427598 | AA406057 | Hs.97998 | ESTs | 1.06 | 2.05 |
| 412565 | M85975 | Hs.344069 | gb: EST02500 Fetal brain, Stratagene (cat | 1.24 | 2.59 |
| 422043 | AL133649 | Hs.110953 | retinoic acid induced 1 | 0.48 | 0.41 |
| 421814 | L12350 | Hs.108623 | thrombospondin 2 | 0.48 | 2.45 |
| 413645 | AA130992 | | gb: zo15e02.s1 Stratagene colon (937204) | 1.32 | 2.45 |
| 435563 | AF210317 | Hs.95497 | solute carrier family 2 (facilitated glu | 0.39 | 0.28 |
| 452396 | H10302 | Hs.112577 | ESTs | 1.60 | 2.45 |
| 440612 | BE561384 | | gb: 601344969F1 NIH_MGC_8 Homo sapiens cD | 1.08 | 2.60 |
| 454721 | AW815588 | | gb: QV0-ST0216-061299-066-a09 ST0216 Homo | 1.44 | 1.65 |
| 417796 | AA206141 | Hs.6786 | ESTs | 1.68 | 3.85 |
| 432864 | D16217 | Hs.279607 | calpastatin | 0.43 | 0.35 |
| 454480 | AA088375 | Hs.22612 | hypothetical protein DKFZp566D1346 | 2.19 | 1.91 |
| 434490 | AF143870 | Hs.15246 | ESTs | 2.26 | 2.07 |
| 418797 | AA515814 | | gb: ng64b03.s1 NCI_CGAP_Lip2 Homo sapiens | 1.42 | 2.55 |
| 403871 | | | C5001783*: gi\|780367\|gb\|AAB05844.1\| (L416 | 1.60 | 2.63 |
| 441283 | AA927670 | Hs.131704 | ESTs | 1.31 | 3.63 |
| 442250 | AW290871 | Hs.129121 | ESTs | 1.14 | 2.38 |
| 456747 | AL037357 | Hs.125864 | tropomodulin 2 (neuronal) | 1.61 | 1.26 |
| 425757 | AA363171 | | gb: EST72986 Ovary II Homo sapiens cDNA 5 | 1.29 | 2.95 |
| 405494 | | | C2001837*: gi\|12697903\|dbj\|BAB21770.1\|(A | 2.09 | 1.00 |
| 432250 | AA452088 | Hs.274170 | Opa-interacting protein 2 | 1.26 | 2.71 |
| 431911 | AK000156 | Hs.272193 | Homo sapiens cDNA FLJ20149 fis, clone CO | 1.46 | 2.60 |
| 413923 | AI733852 | Hs.199957 | ESTs | 1.62 | 2.10 |
| 449590 | AA694070 | Hs.268835 | ESTs | 1.20 | 2.53 |
| 438467 | AA808027 | Hs.123277 | ESTs | 1.48 | 2.10 |
| 432121 | AI824879 | Hs.211286 | ESTs, Weakly similar to 1207289A reverse | 1.27 | 3.13 |
| 412298 | AW936300 | | gb: QV4-DT0021-281299-070-a04 DT0021 Homo | 1.42 | 2.60 |
| 408519 | AA679082 | Hs.43481 | hypothetical protein DKFZp564K192 | 1.84 | 3.70 |
| 416067 | T79732 | Hs.14633 | ESTs | 1.11 | 3.08 |
| 420497 | AW206285 | Hs.253548 | ESTs | 1.90 | 2.48 |
| 405704 | | | NM_001844*: Homo sapiens collagen, type I | 1.42 | 2.90 |
| 423443 | AI432601 | Hs.168812 | Homo sapiens cDNA FLJ14132 fis, clone MA | 1.42 | 2.03 |
| 415904 | Z44679 | Hs.336391 | ESTs | 1.62 | 2.94 |
| 413786 | AW613780 | Hs.13500 | ESTs | 0.33 | 0.17 |
| 404031 | | | C5001700*: gi\|9256616\|ref\|NP_061761.1\|pr | 1.94 | 2.29 |
| 457412 | N40711 | Hs.333300 | hypothetical protein FLJ14026 | 1.92 | 3.20 |
| 439719 | AF086554 | Hs.326048 | Homo sapiens mRNA; cDNA DKFZp434M0420 (f | 1.62 | 2.30 |
| 418161 | AI950754 | Hs.81716 | ESTs | 1.81 | 2.42 |
| 425894 | AW954011 | Hs.180711 | ESTs | 0.92 | 2.20 |
| 419988 | W39388 | Hs.55336 | Homo sapiens, clone MGC: 17421, mRNA, com | 1.34 | 2.57 |
| 439668 | AI091277 | Hs.302634 | frizzled (Drosophila) homolog 8 | 1.67 | 2.66 |
| 450177 | AI698091 | Hs.107845 | ESTs | 1.50 | 2.25 |
| 459704 | AA719572 | Hs.274441 | Homo sapiens mRNA; cDNA DKFZp434N011 (fr | 1.27 | 3.35 |
| 410357 | AW663614 | | gb: hj22e04.x1 NCI_CGAP_Li8 Homo sapiens | 0.69 | 0.59 |
| 459234 | AI940425 | | gb: CM0-CT0052-150799-024-c04 CT0052 Homo | 1.67 | 2.08 |
| 421313 | NM_014923 | Hs.103329 | KIAA0970 protein | 0.57 | 0.26 |
| 431322 | AW970622 | | gb: EST382704 MAGE resequences, MAGK Homo | 1.80 | 2.73 |
| 423086 | AB028984 | Hs.123420 | KIAA1061 protein | 0.40 | 0.56 |
| 425980 | AA366951 | | gb: EST77963 Pancreas tumor III Homo sapi | 1.33 | 2.50 |
| 423185 | BE299590 | Hs.125078 | ornithine decarboxylase antizyme 1 | 0.60 | 0.56 |
| 410840 | AW806924 | | gb: QV4-ST0023-160400-172-h10 ST0023 Homo | 1.50 | 2.88 |
| 403917 | | | Target Exon | 1.82 | 2.02 |
| 437384 | AI674710 | Hs.174397 | ESTs | 1.26 | 2.05 |
| 444389 | AW439340 | Hs.189720 | ESTs | 1.26 | 2.13 |
| 443318 | AI051603 | Hs.133141 | ESTs | 1.46 | 2.20 |
| 441093 | AI698138 | Hs.126918 | ESTs | 1.40 | 2.35 |
| 439432 | AI984203 | Hs.57874 | ESTs | 0.88 | 2.18 |
| 454629 | AW811114 | | gb: MR2-ST0131-111199-016-a04 ST0131 Homo | 1.96 | 2.31 |
| 406207 | | | Target Exon | 2.77 | 2.55 |
| 444872 | AI936264 | | p30 DBC protein | 1.48 | 2.45 |
| 401908 | | | C17000154: gi\|12003980\|gb\|AAG43830.1\|AF21 | 1.15 | 2.28 |
| 404730 | | | Target Exon | 1.84 | 2.78 |
| 457498 | AI732230 | Hs.191737 | ESTs | 1.49 | 2.55 |
| 448471 | AA158617 | Hs.21276 | collagen, type IV, alpha 3 (Goodpasture | 0.37 | 0.36 |
| 438978 | AI095207 | Hs.307972 | ESTs | 1.57 | 2.39 |
| 418786 | AI796317 | Hs.203594 | Homo sapiens uncharacterized gastric pro | 2.86 | 3.34 |
| 400416 | AF083130 | | Homo sapiens CATX-14 mRNA, partial cds | 2.03 | 1.55 |
| 450446 | AI696334 | Hs.14450 | ESTs | 1.32 | 2.38 |
| 419791 | AI579909 | Hs.105104 | ESTs | 0.41 | 0.27 |
| 449436 | AA860329 | Hs.279307 | hypothetical protein DKFZp434I2117 | 2.01 | 1.50 |
| 430808 | L08603 | Hs.247980 | melanocortin 4 receptor | 1.09 | 2.18 |
| 443116 | AI033397 | Hs.132225 | ESTs | 1.30 | 2.25 |
| 437923 | BE088433 | Hs.334696 | hypothetical protein KIAA1335 | 1.40 | 2.50 |
| 403294 | | | Target Exon | 0.98 | 2.18 |
| 436007 | AI247716 | Hs.232168 | ESTs | 1.38 | 1.00 |
| 430649 | AB040941 | Hs.247713 | KIAA1508 protein | 1.52 | 2.85 |
| 437271 | AL137445 | Hs.28846 | Homo sapiens mRNA; cDNA DKFZp566O134 (fr | 1.29 | 2.59 |
| 444500 | AV651273 | Hs.282966 | ESTs, Moderately similar to 2109260A B c | 1.22 | 2.05 |
| 447434 | R16890 | Hs.137135 | ESTs | 1.72 | 2.85 |
| 400830 | | | NM_025006: Homo sapiens hypothetical prot | 2.04 | 2.68 |
| 428114 | AI821548 | Hs.98363 | ESTs, Weakly similar to I38022 hypotheti | 1.09 | 2.74 |
| 409688 | AI150485 | | gb: qf36a10.x1 Soares_testis_NHT Homo sap | 1.67 | 1.38 |
| 440781 | BE561823 | Hs.281434 | Homo sapiens cDNA FLJ14028 fis, clone HE | 1.28 | 2.50 |
| 442662 | U78168 | Hs.8578 | Rap 1 guanine-nucleotide-exchange factor | 1.92 | 2.28 |
| 443078 | M78728 | Hs.132694 | Homo sapiens cDNA: FLJ23149 fis, clone L | 1.42 | 2.03 |
| 440179 | AI990151 | Hs.125904 | ESTs | 1.49 | 2.63 |
| 446780 | R31107 | | gb: yh61g01.s1 Soares placenta Nb2HP Homo | 1.96 | 2.78 |
| 444173 | AI126432 | Hs.149493 | ESTs | 1.50 | 2.10 |
| 417939 | R53863 | Hs.337512 | ESTs, Weakly similar to ALUB_HUMAN !!!! | 1.60 | 2.03 |
| 428490 | BE301738 | Hs.49806 | ESTs, Weakly similar to A46010 X-linked | 0.47 | 0.44 |
| 443869 | AI141520 | Hs.151464 | ESTs, Weakly similar to ALUC_HUMAN !!!! | 1.25 | 2.68 |
| 426322 | J05068 | Hs.2012 | transcobalamin I (vitamin B12 binding pr | 2.12 | 1.15 |
| 411630 | U42349 | Hs.71119 | Putative prostate cancer tumor suppresso | 0.64 | 0.48 |
| 454701 | AW854930 | | gb: PM0-CT0263-201099-003-f06 CT0263 Homo | 1.30 | 2.33 |
| 439795 | N77294 | Hs.194294 | ESTs | 1.17 | 2.33 |
| 425546 | BE409762 | Hs.26118 | hypothetical protein MGC13033 | 1.17 | 2.85 |
| 411245 | AW833441 | | gb: QV4-TT0008-271099-020-g01 TT0008 Homo | 1.90 | 3.98 |
| 434957 | AF283775 | Hs.35380 | x 001 protein | 0.47 | 0.41 |
| 425724 | AA362525 | | gb: EST72223 Namalwa B cells I Homo sapie | 1.38 | 2.63 |
| 446847 | T51454 | Hs.82845 | Homo sapiens cDNA: FLJ21930 fis, clone H | 0.34 | 0.28 |
| 453216 | AL137566 | Hs.32405 | Homo sapiens mRNA; cDNA DKFZp586G0321 (f | 1.28 | 2.19 |
| 421718 | AL117574 | | Homo sapiens mRNA; cDNA DKFZp434L2221 (f | 2.04 | 1.79 |
| 415924 | H18047 | Hs.335821 | ESTs | 2.02 | 3.17 |
| 450850 | AA648886 | Hs.151999 | ESTs | 1.68 | 2.45 |
| 443153 | AI371823 | Hs.34079 | ESTs | 1.13 | 2.41 |
| 434420 | AA688278 | Hs.194864 | hypothetical protein FLJ22578 | 1.34 | 2.38 |
| 426126 | AL118747 | Hs.26691 | ESTs | 1.31 | 2.25 |
| 421926 | AA300591 | | gb: EST13437 Testis tumor Homo sapiens cD | 1.48 | 2.40 |
| 459563 | AI590487 | Hs.49760 | gb: tt77d04.x1 NCI_CGAP_HSC3 Homo sapiens | 1.74 | 3.33 |
| 453006 | AI362575 | Hs.303171 | ESTs | 1.17 | 2.24 |
| 437223 | C15105 | Hs.330716 | Homo sapiens cDNA FLJ14368 fis, clone HE | 0.54 | 0.46 |
| 417016 | AA837098 | Hs.269933 | ESTs | 1.04 | 2.18 |
| 420223 | N27807 | Hs.286 | ribosomal protein L4 | 2.08 | 3.10 |
| 425303 | AA354785 | | gb: EST63098 Jurkat T-cells V Homo sapien | 2.18 | 2.85 |
| 400375 | NM_014115 | | NM_014115*: Homo sapiens PRO0113 protein | 1.83 | 2.14 |
| 456169 | Y07909 | Hs.79368 | epithelial membrane protein 1 | 1.54 | 2.08 |
| 409707 | AA861773 | Hs.313501 | ESTs | 0.79 | 0.84 |
| 422241 | Y00062 | Hs.170121 | protein tyrosine phosphatase, receptor t | 1.46 | 2.06 |
| 443152 | AI803470 | Hs.204529 | KIAA1806 protein | 1.07 | 2.43 |
| 452714 | AW770994 | Hs.30340 | hypothetical protein KIAA1165 | 0.45 | 0.34 |
| 415110 | H04043 | | gb: yj45c03.r1 Soares placenta Nb2HP Homo | 1.62 | 2.07 |
| 443251 | BE185436 | Hs.278839 | ESTs | 1.34 | 2.05 |
| 433441 | R37094 | Hs.13742 | ESTs | 1.76 | 2.05 |
| 434612 | R76513 | Hs.301183 | molecule possessing ankyrin repeats indu | 0.83 | 2.88 |
| 417807 | R17806 | Hs.269452 | gb: yg09b06.r1 Soares infant brain 1NIB H | 1.30 | 2.23 |
| 426902 | AI125334 | Hs.97408 | ESTs | 1.94 | 2.20 |
| 436028 | AA731124 | Hs.120931 | ESTs | 2.01 | 1.73 |
| 428878 | AA436884 | Hs.48926 | ESTs | 1.22 | 2.17 |
| 439749 | AL389942 | Hs.157752 | Homo sapiens mRNA full length insert cDN | 1.32 | 2.75 |
| 442435 | AI986208 | Hs.244760 | ESTs, Highly similar to B34087 hypotheti | 2.09 | 3.13 |
| 416527 | T62507 | Hs.11038 | ESTs | 1.66 | 2.12 |
| 441808 | AW118601 | Hs.127887 | ESTs, Moderately similar to 1609195B blo | 1.22 | 2.58 |
| 417054 | AF017060 | Hs.174151 | aldehyde oxidase 1 | 2.57 | 1.48 |
| 446636 | AC002563 | Hs.15767 | citron (rho-interacting, serine/threonin | 1.16 | 2.23 |
| 418442 | AI873471 | Hs.186898 | ESTs | 1.39 | 2.26 |
| 416640 | BE262478 | Hs.79404 | neuron-specific protein | 0.31 | 0.26 |
| 403146 | | | Target Exon | 1.49 | 2.18 |
| 457397 | AW969025 | Hs.109154 | ESTs | 1.32 | 2.26 |
| 439189 | AI951185 | Hs.144630 | nuclear receptor subfamily 2, group F, m | 1.76 | 2.90 |
| 423969 | AI830571 | Hs.34969 | hypothetical protein DKFZp566N034 | 1.18 | 2.00 |
| 459683 | AI674906 | Hs.199460 | gb: wc73f02.x1 NCI_CGAP_Pan1 Homo sapiens | 1.74 | 2.00 |
| 426826 | AK001890 | Hs.172654 | guanine nucleotide binding protein beta | 2.04 | 1.60 |
| 414462 | BE622743 | Hs.301064 | arfaptin 1 | 0.40 | 0.29 |
| 438027 | N93047 | Hs.19131 | transcription factor Dp-2 (E2F dimerizat | 1.08 | 2.40 |
| 408623 | AW811978 | Hs.254037 | ESTs | 1.64 | 3.08 |
| 433765 | AA909619 | Hs.112668 | ESTs | 1.52 | 2.02 |
| 417132 | N56605 | Hs.269053 | ESTs | 1.64 | 2.51 |
| 416815 | U41514 | Hs.80120 | UDP-N-acetyl-alpha-D-galactosamine: polyp | 0.28 | 0.16 |
| 435186 | AL119470 | Hs.145631 | ESTs | 1.74 | 3.10 |
| 411107 | AW958042 | Hs.95870 | PTD015 protein | 0.49 | 0.24 |
| 406930 | U04691 | | gb: Human olfactory receptor (OR17-219) g | 2.21 | 3.88 |
| 411026 | AW813786 | | gb: RC3-ST0197-120200-015-b05 ST0197 Homo | 1.64 | 1.03 |
| 415766 | H01613 | Hs.50628 | adaptor-related protein complex 4, sigma | 1.64 | 2.51 |
| 446018 | AW631111 | Hs.249727 | gb: hh92e12.y1 NCI_CGAP_GU1 Homo sapiens | 1.56 | 2.48 |
| 440125 | AW238410 | Hs.253888 | ESTs | 1.46 | 2.25 |
| 449832 | AA694264 | Hs.60049 | ESTs | 1.27 | 2.33 |
| 431899 | AA521381 | Hs.187726 | ESTs | 1.11 | 2.53 |
| 431531 | BE142052 | Hs.62654 | kringle-containing transmembrane protein | 1.06 | 2.00 |
| 441077 | AI241273 | Hs.15312 | ESTs | 1.12 | 2.13 |
| 426799 | H14843 | Hs.303154 | popeye protein 3 | 0.61 | 0.51 |
| 419480 | BE536584 | Hs.122546 | hypothetical protein FLJ23017 | 1.88 | 2.38 |
| 455908 | BE156306 | | gb: QV0-HT0367-150200-114-h04 HT0367 Homo | 1.77 | 2.55 |
| 403332 | | | Target Exon | 1.46 | 2.60 |
| 455753 | BE075124 | | gb: PM1-BT0585-110200-003-h02 BT0585 Homo | 1.40 | 2.43 |
| 404429 | | | Target Exon | 1.31 | 2.01 |
| 438941 | AF075047 | Hs.31864 | ESTs | 1.34 | 2.21 |
| 428745 | AA433896 | Hs.201634 | ESTs | 1.72 | 2.06 |
| 411567 | AW851630 | | gb: MR2-CT0222-211099-002-h06 CT0222 Homo | 1.60 | 2.70 |
| 458714 | R20916 | Hs.344777 | ESTs | 0.93 | 2.07 |
| 426839 | M74782 | Hs.172689 | interleukin 3 receptor, alpha (low affin | 1.39 | 2.71 |
| 444539 | AI955765 | Hs.146907 | ESTs, Weakly similar to 2004399A chromos | 1.66 | 2.18 |
| 407322 | AA171892 | Hs.324570 | ESTs, Weakly similar to ALU3_HUMAN ALU S | 1.30 | 2.65 |
| 453826 | AL138129 | | gb: DKFZp547F152_r1 547 (synonym: hfbr1) | 1.52 | 2.73 |
| 435695 | AA694324 | Hs.257675 | ESTs | 1.24 | 2.00 |
| 402294 | | | Target Exon | 1.80 | 3.08 |
| 417759 | R13567 | Hs.12548 | ESTs | 1.63 | 2.58 |
| 417527 | AA203524 | | gb: zx56e10.r1 Soares_fetal_liver_spleen_{—} | 1.52 | 2.02 |
| 427526 | AA405062 | Hs.345830 | gb: zu12e04.r1 Soares_testis_NHT Homo sap | 2.03 | 1.90 |
| 455300 | AW891707 | | gb: CM3-NT0090-040500-171-e02 NT0090 Homo | 1.12 | 2.20 |
| 448121 | AL045714 | Hs.128653 | hypothetical protein DKFZp564F013 | 0.93 | 2.28 |
| 415855 | AI921875 | | gb: wp07e04.x1 NCI_CGAP_Kid12 Homo sapien | 1.43 | 2.08 |
| 425702 | N59555 | | gb: yv76f05.s1 Soares fetal liver spleen | 1.61 | 2.80 |
| 441056 | H37860 | Hs.125720 | ESTs | 1.11 | 2.10 |
| 400311 | AF072164 | Hs.137570 | Homo sapiens HSFE-1 mRNA, partial cds | 2.04 | 2.95 |
| 451478 | NM_012331 | Hs.26458 | methionine sulfoxide reductase A | 0.50 | 0.20 |
| 425288 | AA354502 | | gb: EST62799 Jurkat T-cells V Homo sapien | 0.99 | 2.08 |
| 456397 | W28339 | Hs.150580 | PTD010 protein | 1.11 | 2.29 |
| 405654 | | | C12001521: gi\|7513934\|pir∥T31081 cca3 pr | 2.30 | 1.00 |
| 450151 | AI088196 | Hs.22968 | Homo sapiens clone IMAGE: 451939, mRNA se | 1.21 | 2.60 |
| 419851 | AA287987 | Hs.13477 | ESTs, Weakly similar to 1207289A reverse | 1.26 | 2.60 |
| 406016 | | | Target Exon | 0.57 | 0.48 |
| 440903 | AI468079 | Hs.126623 | ESTs | 2.02 | 1.61 |
| 445026 | W90337 | Hs.282966 | ESTs, Moderately similar to 2109260A B c | 1.56 | 2.23 |
| 414182 | AA136301 | Hs.344442 | KIAA1105 protein | 1.32 | 2.55 |
| 457048 | AA400352 | Hs.112861 | ESTs | 1.54 | 2.05 |
| 440542 | AA889143 | Hs.295655 | ESTs, Weakly similar to PC4259 ferritin | 1.48 | 2.15 |
| 422857 | R71461 | | gb: yi51h07.r1 Soares placenta Nb2HP Homo | 1.42 | 2.78 |
| 445948 | AW444662 | Hs.202247 | ESTs | 1.50 | 2.48 |
| 454002 | BE299567 | Hs.271749 | ESTs, Moderately similar to ALU8_HUMAN A | 1.31 | 2.25 |
| 413656 | T91703 | | gb: ye20g09.s1 Stratagene lung (937210) H | 2.10 | 1.69 |
| 420441 | AI986160 | Hs.180383 | dual specificity phosphatase 6 | 0.99 | 2.33 |
| 412062 | H09124 | Hs.202341 | Homo sapiens cDNA: FLJ23573 fis, clone L | 2.14 | 1.61 |
| 408991 | BE501816 | Hs.281927 | ESTs | 1.76 | 2.83 |
| 432534 | AW361626 | Hs.339833 | hypothetical protein FLJ11240 | 0.41 | 0.28 |
| 435136 | R27299 | Hs.10172 | ESTs | 0.76 | 3.40 |
| 451052 | AA281504 | Hs.24444 | Homo sapiens cDNA: FLJ22165 fis, clone H | 2.16 | 1.85 |
| 413928 | AA442498 | Hs.6700 | ESTs, Moderately similar to Z195_HUMAN Z | 1.30 | 2.00 |
| 439448 | AA970788 | Hs.257586 | ESTs | 1.87 | 2.23 |
| 403344 | | | NM_000341: Homo sapiens solute carrier fa | 1.36 | 2.22 |
| 418056 | AA524886 | | gb: nh34f02.s1 NCI_CGAP_Pr3 Homo sapiens | 1.42 | 2.85 |
| 435428 | AI791746 | Hs.130293 | ESTs | 2.44 | 1.32 |
| 419964 | AA811657 | Hs.220913 | ESTs | 1.32 | 2.08 |
| 440926 | AW196772 | Hs.131323 | ESTs | 1.80 | 2.65 |
| 452625 | AA724771 | Hs.61425 | ESTs | 1.64 | 2.18 |
| 452797 | AI369787 | Hs.7146 | ESTs | 1.47 | 3.16 |
| 436120 | AI248193 | Hs.119860 | ESTs | 1.41 | 2.83 |
| 449567 | AI990790 | Hs.188614 | ESTs | 1.48 | 2.45 |
| 409628 | AB021865 | Hs.55276 | potassium voltage-gated channel, Shal-re | 1.70 | 2.23 |
| 416617 | H69311 | Hs.205980 | ESTs | 1.83 | 2.04 |
| 452266 | AI767250 | Hs.165240 | ESTs | 0.58 | 0.43 |
| 404606 | | | Target Exon | 1.47 | 3.75 |
| 401814 | | | Target Exon | 2.00 | 1.91 |
| 428403 | AI393048 | Hs.326159 | leucine rich repeat (in FLII) interactin | 0.33 | 0.21 |
| 433390 | AA586950 | Hs.260180 | Homo sapiens mRNA; cDNA DKFZp761G18121 ( | 2.00 | 4.90 |
| 451443 | AW295527 | Hs.210303 | ESTs | 1.87 | 2.25 |
| 411188 | BE161168 | | gb: PM0-HT0425-170100-002-a10 HT0425 Homo | 2.15 | 1.69 |
| 452704 | AA027823 | Hs.149424 | Homo sapiens PNAS-130 mRNA, complete cds | 2.64 | 1.65 |
| 424060 | X92108 | | H. sapiens mRNA for subtelomeric repeat s | 2.40 | 2.58 |
| 433331 | AI738815 | Hs.117323 | ESTs | 1.46 | 2.10 |
| 428520 | AA331901 | Hs.184736 | hypothetical protein FLJ10097 | 0.44 | 0.19 |
| 439492 | AF086310 | Hs.103159 | ESTs | 0.42 | 0.26 |
| 426736 | AA431615 | Hs.130722 | ESTs | 1.90 | 2.45 |
| 416225 | AA577730 | Hs.188684 | ESTs, Weakly similar to PC4259 ferritin | 2.72 | 6.25 |
| 404917 | | | Target Exon | 1.60 | 2.15 |
| 448955 | AW207597 | Hs.28102 | ESTs | 2.08 | 1.75 |
| 402797 | | | Target Exon | 2.12 | 1.37 |
| 457951 | U23860 | | gb: Human clone mcag19 chromosome 16 CTG | 1.72 | 2.00 |
| 426982 | AA149707 | Hs.173091 | ubiquitin-like 3 | 0.36 | 0.17 |

Pkey: Unique Eos probeset identifier number
ExAccn: Exemplar Accession number, Genbank accession number
UnigeneID: Unigene number
Unigene Title: Unigene gene title
R1: 80th percentile of Ta or T1 tumor Als from patients who upstaged divided by 80th percentile of Ta or T1 tumor Als from patients who did not upstage
R2: median of Ta or T1 tumor Als from patients who upstaged divided by the median of Ta or T1 tumor Als from patients who did not upstage

TABLE 8B

| Pkey | CAT Number | Accession |
|---|---|---|
| 408139 | 10421_1 | AA451966 NM_016370 AB036693 AL139228 R58124 AI634847 AL119333 W07356 AI334284 H29050 |
| | | AI192685 AA652438 AW172843 W19794 N21460 AI743862 AW130622 AA991348 AI204553 AA992664 |
| | | N80848 AA699329 AI824676 R26624 R49653 |
| 408255 | 1049351_1 | AW807321 AW807262 AW177104 AW807319 AW807115 AW807344 AW807324 AW178116 BE141575 |
| | | AW845849 AW807105 AW845868 BE140942 AW807178 AW807167 AW807398 AW807320 AW807306 AW845866 |
| 408283 | 1050275_1 | BE141579 AW807555 AW807502 BE141596 AW845845 AW807500 AW845864 |
| | | AW807480 AW807486 AW807478 AW178109 AW807228 AW807374 AW807125 AW846124 AW807470 |
| | | AW807477 AW807510 AW807208 BE141573 AW807465 |
| 408839 | 1085657_1 | AW277084 R27662 R26970 D79194 |
| 409126 | 110159_1 | AA063426 AW962323 AW408063 AA063503 AA772927 AW753492 BE175371 AA311147 |
| 409282 | 111512_1 | AW966480 AA069840 AA384646 |
| 409291 | 1115745_1 | AW373472 AW373484 BE071899 BE071898 |
| 409367 | 1123651_1 | AW382767 BE153835 BE153702 BE153572 |
| 409688 | 114831_3 | AI150485 AW938392 AA076894 AW883422 |
| 409692 | 114869_1 | AI500724 AA399661 AA397891 AI471084 AI423511 T07531 AI094336 |
| 409894 | 1157906_1 | BE081731 AW861738 AW503629 BE081969 |
| 410154 | 117950_1 | F06959 Z43559 AA082002 |
| 410357 | 1197159_1 | AW663614 BE046540 BE045760 AW827443 BE046544 |
| 410642 | 1213736_1 | AW792784 Z44444 H06639 |
| 410725 | 1218207_1 | AW799279 AW799395 AW799392 AW799276 AW799479 H57885 |
| 410730 | 121847_1 | AW368860 AA457091 AI903441 AA088823 W88852 AW979154 AA826016 R94779 |
| 410744 | 1219485_1 | H86002 W92289 AW801558 AW801324 AW801270 AW801307 AW801351 AW801357 AW801299 |
| | | AW801609 AW801356 AW801420 AW801425 AW801358 AW801429 AW801428 AW801427 AW801424 AW801306 |
| 410840 | 1223800_1 | AW806924 AW866537 AW866473 AW866298 AW866390 AW866478 AW866454 AW866309 AW866539 |
| | | AW866521 AW866547 AW866517 AW866403 AW866369 |
| 410956 | 1227882_1 | AW938322 AW938307 AW938320 AW938323 AW811840 |
| 411008 | 1229027_1 | AW813238 AW813474 AW813334 AW816081 AW813296 AW813363 AW813397 AW813327 AW813328 |
| | | AW816031 AW816140 |
| 411026 | 1229373_1 | AW813786 |
| 411141 | 1233793_1 | AW819561 AW819682 AW819563 AW819688 AW819499 AW819498 AW819690 BE065081 |
| 411184 | 1234977_1 | AW821117 AW855541 AW855405 AW855374 |
| 411188 | 1235093_1 | BE161168 BE162466 AW821260 |
| 411245 | 1236412_1 | AW833441 AW833552 AW833700 AW833610 AW833673 AW833675 |
| 411347 | 1239834_1 | AW838126 AW838294 AW838247 AW838251 AW838292 AW838299 AW838374 |
| 411432 | 1245636_1 | AW846272 AW846564 AW846545 AW846285 AW846135 AW846317 AW846200 AW846265 AW846326 |
| | | AW846196 AW846357 AW846153 AW846286 AW846319 AW846277 AW846381 AW846438 AW846481 AW846352 |
| 411496 | 1248073_1 | AW849241 AW849569 AW849243 |
| 411567 | 1249774_1 | AW851630 AW851703 AW851735 AW851723 AW851708 AW851712 |
| 411590 | 125064_1 | T96183 T64070 AA094134 |
| 411608 | 1251259_1 | AW853441 BE145228 BE145218 BE145162 BE145283 |
| 411652 | 1252836_1 | AW855393 AW855560 AW855424 |
| 411880 | 1263110_1 | AW872477 BE088101 T05990 |
| 411918 | 1265807_1 | AW876354 AW876179 AW876318 AW876290 AW876234 AW876125 AW876199 AW876198 |
| 411920 | 1265812_1 | AW876263 AW876257 AW876261 AW876273 AW876231 AW876398 AW876334 AW876134 AW876371 |
| | | AW876308 AW876314 AW876328 AW876169 AW876331 AW876426 AW876407 AW876412 AW876322 AW876363 |
| | | AW876218 AW876240 AW876141 AW876138 AW876326 AW876181 AW876131 AW876378 AW876206 AW876191 |
| | | AW876188 AW876376 AW876289 AW876415 AW876209 AW876366 AW876343 AW876347 AW876165 |
| 412085 | 1276467_1 | AW891667 H93096 |
| 412252 | 1285293_1 | AW903782 AW903668 AW903672 AW903763 AW903784 |
| 412274 | 128647_1 | AA101443 R20332 F07484 |
| 412298 | 1288098_1 | AW936300 AW936538 AW936386 |
| 412302 | 1288128_1 | AW936334 AW936371 AW936474 |
| 412679 | 1321229_1 | BE144762 AW979091 |
| 412902 | 1335166_1 | BE008018 BE008025 BE008026 BE007959 BE007994 BE008016 8E008019 BE008024 BE008022 |
| | | BE008027 BE008029 BE008020 BE008015 BE008021 BE008028 BE008023 BE008030 BE008014 |
| 413035 | 1346295_1 | BE155563 BE155574 BE155556 BE061294 |
| 413068 | 1348104_1 | BE063792 BE063803 BE063775 BE063801 BE063809 BE063777 BE063784 BE063808 BE066125 |
| | | BE063782 BE063805 BE063812 |
| 413196 | 135322_1 | AA127386 R15644 AA127404 |
| 413346 | 136323_1 | AA128586 AA463667 AA235203 |
| 413444 | 1370831_1 | BE141019 BE141473 BE141529 |
| 413489 | 1373392_1 | BE144228 BE144291 |
| 413593 | 137891_1 | AA205248 AA130658 AA204737 |
| 413645 | 138145_1 | AA130992 AA503835 AW969537 |
| 413656 | 1381628_1 | T91703 BE155222 BE155240 BE155274 BE155275 T92469 |
| 413750 | 1386250_1 | BE161453 W28808 |
| 413885 | 1397288_1 | BE177442 BE177439 BE177445 BE177440 BE177448 BE177444 BE177433 |
| 413888 | 139750_1 | AA580288 AA315655 AA133031 AA377748 |
| 414051 | 1413185_1 | BE244127 BE246216 |
| 414623 | 1466952_1 | BE391050 BE389191 BE389697 |
| 415054 | 151827_1 | AI733907 AA159708 AI732614 |
| 415110 | 1522905_1 | H04043 D60988 D60337 |
| 415157 | 1525616_1 | D63257 D78918 D63214 |
| 415361 | 1534970_1 | F06724 Z43690 R21681 |
| 415378 | 1535274_1 | T16964 F07075 H10256 |
| 415449 | 1537026_1 | H15034 T17195 F09069 |
| 415770 | 155437_1 | M79237 AI267298 AA169260 |
| 415806 | 155668_1 | AA169560 AA169840 |
| 415855 | 155930_1 | AI921875 AA170835 AA866613 |
| 415989 | 156454_1 | AI267700 AI720344 AA191424 AI023543 AI469633 AA172056 AW958465 AA172236 AW953397 AA355086 |
| 416311 | 158797_1 | D80529 D81719 C14833 AA179446 AA357794 |
| 416463 | 1595999_1 | H59241 H57540 R00795 |
| 417086 | 164830_1 | AA194446 AA194603 AA193162 AA196396 Z24810 |
| 417117 | 1651230_1 | N46778 W86339 N75221 |
| 417324 | 166714_1 | AW265494 AA455904 AA195677 AW265432 AW991605 AA456370 |
| 417527 | 168576_1 | AA203524 W88451 |
| 417670 | 1692163_1 | R07785 T85948 T86972 |
| 417882 | 1705458_1 | R22311 R23982 R23997 |
| 417890 | 1706141_1 | R79048 R23111 |
| 418056 | 171841_1 | AA524886 AW971347 AA211537 |
| 418797 | 179095_1 | AA515814 AA515037 AA230024 AA228343 |
| 418859 | 179717_1 | AA229558 AA345492 AA229582 |
| 419145 | 182217_1 | N99638 AW973750 AA328271 H90994 AA558020 AA234435 N59599 R94815 |
| 419386 | 184356_1 | AA236867 AA237066 AA354236 AW957759 H08961 |
| 419501 | 185354_1 | AW843822 AW844020 AA825923 AW968657 AW854173 BE084673 |
| 421338 | 201378_1 | AA287443 AA419385 BE084078 AI478347 |
| 421582 | 2041_1 | AI910275 X00474 X52003 X05030 NM_003225 AA314326 AA308400 AA506787 AA314825 |
| | | AI571948 AA507595 AA614579 AA587613 R83818 AA568312 AA614409 AA307578 AI925552 AW950155 |
| | | AI910083 M12075 BE074052 AW004668 AA578674 AA582084 BE074053 BE074126 BE074140 AA514776 |
| | | AA588034 BE074051 BE074068 AW009769 AW050690 AA858276 R55389 AI001051 AW050700 AW750216 |
| | | AA614539 BE074045 AI307407 AW602303 BE073575 AI202532 AA524242 AI970839 AI909751 BE076078 |
| | | AI909749 R55292 |
| 421718 | 20595_1 | AL117574 AI681945 BE503055 AW015330 C16652 AA441855 AA329853 AW962502 AA441830 AF114049 |
| 421926 | 209246_1 | AA300591 AW963893 AA300493 |
| 422259 | 214322_1 | AA307584 AW795791 AW795790 |
| 422297 | 214851_1 | AW961290 AA308338 R60841 BE378716 R13001 |
| 422337 | 215447_1 | R38572 R49597 H51730 F10468 AA309198 BE011889 AA613236 |
| 422444 | 216595_1 | AA310688 AA355321 AW962134 |
| 422857 | 222190_1 | R71461 AA328744 AA318128 BE081817 BE081587 BE081751 |
| 422936 | 223099_1 | AA319278 AA319712 AW961504 AW954956 |
| 422940 | 223106_1 | BE077458 AA337277 AA319285 |
| 422959 | 22327_3 | AV647015 AV647162 AA477047 AW392066 BE168052 AA657684 AL040747 AL043589 AL041618 |
| | | W24630 AA419002 AA778650 AI028269 AW378085 AW780241 AI984255 AW129462 AI271476 AL040967 AA836991 |
| | | AL040910 AW273346 AA132452 AI375545 AI436321 N53166 AA598667 AA423804 AA889495 AI954063 AI923968 |
| | | AW194576 AI400578 AA748499 AA860230 AW519209 AA339537 AA476935 AA767391 AI017695 AI860419 N93437 |
| | | AW452389 AI051533 AA806940 AA609569 AI624860 AI356952 AI811516 AI439198 AA749268 AI206880 AI784422 |
| | | AI431957 AI187038 AI678429 AI273421 AI358604 AI691155 AA586499 AA897667 AA524576 AI417772 AW799936 |
| | | BE241923 AA013428 BE164987 AW602710 AI933540 AA757772 AW015132 AI954159 AW956377 W69468 AI807580 |
| | | AI871746 AI922524 N80292 AI676213 BE467086 N92405 AV656311 AI678172 AW339455 AI949278 AI872882 |
| | | AI123785 AA016267 AI961623 AW168519 F28686 F37181 AA908333 AW020788 AW374027 |
| 423074 | 22470_1 | AL109963 AA134692 BE273642 M78295 AI825179 AA228029 BE538677 AW589948 |
| | | AW241564 AA732224 AA228030 AW977225 |
| 423622 | 230333_1 | BE154847 BE154890 AA328702 |
| 423736 | 231511_1 | AW936874 AW936923 AW936924 AA330274 |
| 423977 | 233868_1 | AA333232 AW962695 R54896 |
| 424060 | 23490_1 | X92108 AW295478 AI768675 AI086644 AW190160 AA313783 AA322741 AA354698 |
| | | AW962099 AL044667 AA282648 AA973897 AL036586 AA386055 AL135179 AW882199 |
| 424160 | 236151_1 | T74062 F12441 AA336207 BE185031 |
| 424290 | 237857_1 | AA338396 AW966247 AW753612 |
| 424951 | 245273_1 | AW964082 AA348838 AA348839 |
| 425288 | 249569_1 | AA354502 AA355215 AW962122 |
| 425303 | 249780_1 | AA354785 AA354792 AW958427 |
| 425702 | 255206_1 | N59555 AA362113 AA362136 |
| 425724 | 255509_1 | AA362525 AW979199 AA857501 |
| 425757 | 255956_1 | AA363171 AW963347 AA371863 |
| 425893 | 257742_1 | AA629695 AA365582 AA365581 AW959799 AW876559 AW876588 |
| 425980 | 258778_1 | AA366951 AA470999 AA469425 |
| 426123 | 261304_1 | AA370352 AA370860 AW962776 |
| 426477 | 267804_1 | AA379464 AA379611 AA379463 |
| 428036 | 28620_1 | AW068302 AI754558 AI750727 AI752631 AA302174 AA327522 M64110 AW859944 AW859989 AI751995 AA769620 |
| | | AI858829 AI924875 AI888836 AA864291 AI685060 AW088029 AI924908 AW466328 AI093800 AA991651 |
| | | AI254501 BE004703 AA334442 AW938852 AA194330 AL046953 AA852866 AW391995 W30846 AW662928 W25261 |
| | | AA042863 R99045 H97060 W03910 H94687 T88984 AL048165 T29632 N31556 N36484 AI798679 AA989355 W23832 |
| | | AA873789 AI743646 AA363587 AI814748 AW338990 N73740 N83666 AL047816 R24137 R63433 |
| | | AA524984 AA234043 AA195131 N99903 AA453669 AI240302 AA370271 AI950026 AW771049 AA121476 AA569557 |
| | | AI752632 AI355594 AI471993 AI159941 N94555 AI753138 N21537 H97881 N25769 AW068044 AA808425 R63380 |
| | | AA384736 AA384738 AA852352 AI073645 AA527960 AA525036 AA044414 AI752460 AA703064 R01216 AA897183 |
| | | AI751996 T81078 H95047 AA573642 D58348 N20953 AA437143 N95439 AA579540 AW867056 AA770090 AI085180 |
| | | AI806799 AA426421 AI572513 R24081 AA853189 AA295620 AA234044 AA371020 AW994984 H20896 AW964438 |
| | | AA318516 AA318499 AA318727 AA318211 AA318478 AA318444 AA318307 AA318497 AA318448 AA318309 |
| | | AA318496 AA318213 AA318435 AA318424 AA318217 AA318523 AA318438 AA318487 AA318724 AA593185 |
| | | AW994985 T69842 AI251813 AA478174 AA447737 T68350 F07712 AA121145 H08973 AA345212 BE000667 |
| | | AW068210 AW608407 R05674 H16712 N85426 N42354 H85516 BE147991 T28113 R32662 AA384678 AW239275 |
| | | H82382 AW840700 D58229 C04082 W45394 AW795667 R73973 BE002409 AA042828 AA363555 AJ223812 |
| | | AA344709 BE149590 R70995 W46881 W90778 N71242 AA534826 AL040676 R23797 H96450 |
| | | AA062957 D79947 W46960 AW959278 AA295997 AA026215 AW579469 AW365135 AW365134 AW994353 |
| | | AW972886 AW069166 AA343690 AW888731 AI751527 AA937490 AA937506 AI826715 BE465604 AI925532 |
| | | AI858109 AW339097 AI858524 AI720571 BE046506 AW384981 AA043908 AA375983 AA525181 AW068366 |
| | | AW070577 AW891837 N83985 AW182753 AI422979 AI679733 BE006555 AL048166 AI081401 AI888821 |
| | | AI626043 N37087 AI624140 AI801298 AA600048 AI753947 H89615 N66424 AW069756 AI814880 AI982806 |
| | | AI754287 AI971816 AW069022 AW069069 AW069454 AA342989 AI077712 AI311467 AI087361 AI801015 |
| | | W46993 AI281324 AW191963 AI421675 AI300881 AI356670 AA873156 AI004219 AI189685 AA478018 |
| | | AA076063 AI445222 AI753124 AI521569 AI925026 AI022368 AI475993 H20846 AI223234 AI635123 |
| | | AA579170 N30442 AW117889 AA807935 AA558975 AI306636 AA888963 AI952591 AI935835 AI445293 |
| | | H16713 AW139833 AA622122 AA972051 AI280828 H09543 AI453725 AW069613 AI865615 AI753921 |
| | | AI368782 AI633208 AI446651 W46961 N22201 H82276 C16555 AA291477 AW440535 AW517755 AA669921 |
| | | AI926777 AW662118 AA553369 N67873 AW023948 C15861 C16601 AI251465 AW079187 BE045090 AI273006 |
| | | C16390 C16503 AI620823 F13661 N66864 Z21311 C16108 C16089 C16400 AA758273 AI287781 AA864676 |
| | | AW608074 AW385583 AI589944 AA665817 AW192979 AW469065 AA564048 H84715 C16417 AA731072 AA661674 |
| | | C16487 N29477 AW189997 AI370492 C16471 AA652809 AA936687 AA506512 C16306 AW028413 AI537935 |
| | | AA528347 C16255 AW029046 C16202 AI868152 AI524662 T94414 AI567041 AI619654 AW008486 AI075624 |
| | | AA577434 AA345104 T30105 AA932002 C16585 AI750390 AW294265 AI619552 AA669781 AA026678 AW132002 |
| | | AW263919 C16562 AA759137 AA693351 Z40779 C16577 AA885045 AW073763 R45484 AI520895 U54708 T49285 |
| | | AI568126 AW006569 AI093317 AL119781 T61046 AI053563 H51958 AF114144 AA305739 AW950394 AW793928 |
| | | AW793910 AL047737 AV659047 AV659632 AI750389 AA092053 AA092798 H85367 T61597 R23745 Z20418 T78485 |
| | | AI751528 AW068121 AA853188 AI752459 AA853711 AW950663 R78964 R36359 R21626 R21522 |
| 428518 | 292383_1 | AW969656 AA501412 AA905186 AA429703 AA431958 |
| 429321 | 302955_1 | AA449921 AA449922 C75309 |
| 429468 | 30498_1 | AF033579 NM_005995 |
| 430068 | 312849_1 | AA464964 M85405 AA947566 |
| 430439 | 31808_1 | AL133561 AL041090 AL117481 AL122069 AW439292 AI968826 |
| 431089 | 327825_1 | BE041395 AA491826 AA621946 AA715980 AA666102 |
| 431322 | 331543_1 | AW970622 AA503009 AA502998 AA502989 AA502805 T92188 |
| 431926 | 339082_1 | AW972724 AA522631 AA877998 |
| 432222 | 343347_1 | AI204995 AW827539 AW969908 AW440776 AA528756 |
| 432407 | 34624_1 | AA221036 R87170 BE537068 BE544757 C18935 AW812058 T92565 |
| | | AA227415 AA233942 AA223237 AA668403 AA601627 AW869639 BE061833 BE000620 AW961170 AW847519 |
| | | AA308542 AW821833 AW945688 C04699 AA205504 AA377241 AW821667 AA055720 AW817981 AW856468 |
| | | AA155719 AA179928 T03007 AW754298 AA227407 AA113928 AA307904 C16859 |
| 432586 | 350432_1 | AA568548 AI050036 AA554053 AI911659 AI826259 |
| 433010 | 357372_1 | AW970018 AA573669 AA573622 R08736 |
| 433436 | 366107_1 | AW162474 AA588442 AI972440 |
| 434414 | 38585_1 | AI798376 S46400 AW811617 AW811616 W00557 BE142245 AW858232 AW861851 |
| | | AW858362 AA232351 AA218567 AA055556 AW858231 AW857541 AW814172 H66214 AW814398 AF134164 |
| | | AA243093 AA173345 AA199942 AA223384 AA227092 AA227080 T12379 AA092174 T61139 AA149776 |
| | | AA699829 AW879188 AW813567 AW813538 AI267168 AA157718 AA157719 AA100472 AA100774 AA130756 |
| | | AA157705 AA157730 AA157715 AA053524 AW849581 AW854566 C05254 AW882836 T92637 AW812621 AA206583 |
| | | AA209204 BE156909 AA226824 AI829309 AW991957 N66951 AA527374 H66215 AA045564 AI694265 H60808 |
| | | AA149726 AW195620 BE081333 BE073424 AW817662 AW817705 AW817703 AW817659 BE081531 H59570 |
| 434830 | 39406_1 | AW852235 AF156166 R07008 |
| 435073 | 399701_1 | AA664078 AW363313 AA805009 |
| 436608 | 42361_3 | AA628980 AI126603 BE504035 |
| 437046 | 43210_1 | BE149154 BE149151 X60138 |
| 437272 | 435447_1 | AW975957 AA747943 AA811289 |
| 437908 | 445001_1 | AI082424 AI740586 AA771764 AA771806 AI033879 BE500996 AW204531 D38676 |
| 438089 | 44963_1 | W05391 X56197 H75313 AW770789 AA281642 AV654440 AA344646 R00244 T83378 |
| 438091 | 44964_1 | AW373062 T55662 AI299190 BE174210 AW579001 H01811 W40186 R67100 AI923886 AW952164 AA628440 |
| | | AW898607 AW898616 AA709126 AW898628 AW898544 AA947932 AW898625 AW898622 AI276125 AI185720 |
| | | AW510698 AA987230 T52522 BE467708 AW243400 AW043642 AI288245 AI186932 D52654 D55017 D52715 |
| | | D52477 D53933 D54679 AI298739 AI146984 AI922204 N98343 BE174213 AA845571 AI813854 AI214518 |
| | | AI635262 AI139455 AI707807 AI698085 AW884528 AI024768 AI004723 AW087420 AI565133 N94964 AI268939 |
| | | AW513280 AI061126 AI435818 AI859106 AI360506 AI024767 AA513019 AA757598 X56196 AA902959 AI334784 |
| | | AI860794 AA010207 AW890091 AW513771 AI951391 AI337671 T52499 AA890205 AI640908 H75966 AA463487 |
| | | AA358688 AI961767 AI866295 AA780994 AI985913 BE174196 AA029094 AW592159 T55581 N79072 AI611201 |
| | | AA910812 AI220713 AW149306 AI758412 AA045713 R79750 N76096 |
| 438535 | 45946_1 | L09078 L03145 L09094 L09098 L03165 L09102 |
| 438813 | 46553_1 | M27346 |
| 439590 | 47413_1 | AF086410 W94386 W74609 |
| 439848 | 477806_1 | AW979249 D63277 AA846968 |
| 440612 | 49847_1 | BE561384 AW732707 |
| 440642 | 499098_1 | AI744995 AI973099 R19291 AA935215 AA897534 AI698070 BE179102 AI694912 |
| 441846 | 527227_1 | AW850980 AA969613 AW366793 |
| 443100 | 559752_1 | AI033188 BE004743 AW804074 BE004795 BE178660 BE089438 BE089378 BE089439 |
| | | BE089437 BE089423 BE089420 BE089425 BE089424 BE089426 |
| 443197 | 56261_1 | Z43613 AA320191 AW954963 H15889 R83872 |
| 443357 | 567506_1 | AW016773 AI052778 AI452937 AW085293 |
| 444163 | 593658_1 | AI126098 AI184746 AI148521 |
| 444298 | 600160_1 | Z17870 AL039867 AI139927 |
| 444312 | 600647_1 | R44007 N54521 AI140476 AW418649 T95308 |
| 444872 | 623948_1 | AI936264 AI219228 AI200740 BE300771 AI635739 AI690626 AW044010 R60842 R40388 |
| 445468 | 640681_1 | AW450439 AW297340 AI239849 AI613119 |
| 445555 | 64334_1 | AW974013 AA557257 N36812 AI597998 AA873565 |
| 445689 | 647829_1 | BE158869 AI248241 BE158865 BE158868 |
| 446780 | 692897_1 | R31107 AI341136 AI653198 H04953 |
| 447008 | 70358_1 | BE010189 AW879041 BE008038 AW905325 AA343575 AW844209 AL038020 BE010133 BE010083 |
| | | AW903901 BE174377 AI309717 AI349651 AI903726 BE176661 AW905394 |
| 448489 | 765247_1 | AI523875 R45782 R45781 |
| 450717 | 844561_1 | T94709 AI820675 AI732253 |
| 450724 | 844585_1 | R55428 AI820704 AI732283 R54983 |
| 450735 | 844617_1 | AI732321 R55640 R55639 AI820744 |
| 451724 | 882130_1 | AI903765 AI811194 BE007147 AW130760 |
| 452565 | 922372_1 | BE066552 BE066341 AI907683 |
| 453577 | 972216_1 | AL043049 T95976 |
| 453823 | 982526_1 | AL137967 BE064160 BE064186 |
| 453826 | 982669_1 | AL138129 AL138179 BE064231 |
| 454457 | 1207274_1 | AW753456 AW753036 AW854868 AW854862 |
| 454523 | 1221564_1 | AW803980 AW803974 |
| 454609 | 1226517_1 | AW810204 AW810555 AW810196 AW810619 AW810507 |
| 454629 | 1227240_1 | AW811114 AW811095 AW811087 AW811124 AW811054 AW811094 AW811157 |
| 454631 | 1227443_1 | AW811324 AW811325 AW811326 AW811333 AW811329 AW811328 AW811332 AW811339 AW811335 |
| 454639 | 1227728_1 | AW811633 AW811652 AW811898 |
| 454662 | 1228537_1 | AW812715 AW812646 AW812714 |
| 454701 | 1229702_1 | AW854930 AW814431 AW814190 AW814115 AW854941 |
| 454721 | 1230747_1 | AW815588 AW815671 AW815493 AW815898 AW815613 AW815490 AW815808 AW815426 |
| 454741 | 1232559_1 | BE154396 AW817959 BE154393 |
| 454826 | 1236377_1 | AW833676 AW833814 AW833798 AW833677 AW833449 AW833630 AW833626 AW833444 AW833366 |
| | | AW833791 AW833659 AW833432 AW833534 AW833556 AW833553 |
| 454915 | 1242343_1 | AW841619 AW851958 AW851851 AW851985 |
| 455065 | 1251980_1 | AW854352 AW854311 AW854340 AW854461 |
| 455087 | 1252832_1 | AW855389 AW855556 AW855420 |
| 455116 | 1254206_1 | AW857271 AW857308 AW857296 AW857258 |
| 455236 | 1265662_1 | AW875972 AW875983 AW875974 AW876000 AW875966 AW876050 |
| 455300 | 1276482_1 | AW891707 AW891696 AW891917 AW891913 AW891912 AW891909 AW891890 AW891892 AW891889 |
| | | AW891697 AW891880 AW891898 |
| 455407 | 1288347_1 | AW936813 AW936731 AW936728 AW936600 AW936681 AW936651 |
| 455508 | 1318507_1 | AW976165 C04000 |
| 455527 | 1322125_1 | AW984479 AW984498 AW984495 AW984477 AW984480 AW984504 |
| 455642 | 1348163_1 | BE063965 BE063968 BE064034 BE064028 BE063874 BE063966 BE063869 BE064043 BE064033 BE063884 |
| 455649 | 1348708_1 | BE065051 BE155165 BE064764 BE155231 BE064648 BE064671 BE064636 |
| 455753 | 1356070_1 | BE075124 BE075229 BE075278 |
| 455757 | 1358657_1 | BE079531 BE079371 BE079372 BE079593 BE079468 BE079504 BE079505 |
| 455908 | 1382301_1 | BE156306 BE156188 BE156298 BE156377 BE156374 |
| 455917 | 1382784_1 | BE156765 BE156770 BE156767 BE156769 BE156803 BE156802 BE156847 BE156853 BE156780 BE156836 |
| | | BE156792 BE156834 BE156779 BE156789 BE156833 BE156844 BE156831 BE156849 BE156797 |
| | | BE156784 BE156801 BE156843 BE156793 BE156852 |
| 455988 | 1397740_1 | BE177983 BE178322 |
| 456034 | 142696_1 | AW450979 AA136653 AA136656 AW419381 AA984358 AA492073 BE168945 AA809054 AW238038 BE011212 |
| | | BE011359 BE011367 BE011368 BE011362 BE011215 BE011365 BE011363 |
| 456172 | 1603643_1 | R99050 R99067 H67642 |
| 456332 | 179104_1 | AA228357 AW841786 AW841716 |
| 456381 | 184123_1 | AA236606 AA459341 AA237079 |
| 457297 | 313764_1 | AW968188 AA468196 AA468269 AA468298 |
| 457389 | 331080_1 | AW970989 AA502167 AA507546 |
| 457567 | 357346_1 | AW939074 AW939073 BE160476 AW939938 AW939206 AW940012 AW939076 AA573577 AW750479 AA574383 |
| | | AW970057 |
| 457581 | 359936_1 | AA578512 AA595535 BE177533 |
| 457741 | 395767_1 | BE044740 AW827360 AW827623 BE161439 BE044718 BE046207 BE046551 AA653908 BE166581 |
| 457871 | 426637_1 | AI168278 AA868238 BE550792 AI522194 AI819707 AA973538 AI990086 AI628424 AI095270 AI991608 AA730741 |
| 457951 | 44251_1 | U23860 U80739 |
| 459234 | 945240_−1 | AI940425 |

Pkey: Unique Eos probeset identifier number
CAT number: Gene cluster number
Accession: Genbank accession numbers

TABLE 8C

| Pkey | Ref | Strand | Nt_position |
|---|---|---|---|
| 400704 | 8118864 | Minus | 63110-63241 |
| 400830 | 8570385 | Plus | 157683-163035 |
| 400834 | 8705192 | Plus | 121963-122288 |
| 400840 | 9188586 | Plus | 113882-114121 |
| 400850 | 1927150 | Minus | 4506-4691 |
| 400881 | 2842777 | Minus | 91446-91603, 92123-92265 |
| 401016 | 8117441 | Plus | 126234-126359, 128050-128236 |
| 401090 | 8492704 | Minus | 201281-201460 |
| 401215 | 9858408 | Plus | 103739-103919 |
| 401241 | 4827300 | Minus | 30503-30844, 31056-31248 |
| 401335 | 9884881 | Plus | 15736-16352 |
| 401381 | 8570226 | Minus | 118629-119146, 119392-119657 |
| 401400 | 7708226 | Minus | 33028-33585 |
| 401469 | 6682292 | Minus | 125521-125639 |
| 401473 | 7249001 | Plus | 115142-117305 |
| 401577 | 9280797 | Minus | 139377-139674, 141195-141281, 142217-142340 |
| 401658 | 9100664 | Plus | 89638-90028 |
| 401659 | 7689875 | Minus | 183379-183521 |
| 401686 | 6468551 | Plus | 5005-5426, 6810-7042 |
| 401723 | 7656694 | Plus | 147273-147503 |
| 401814 | 7408052 | Plus | 136003-136726 |
| 401818 | 7467933 | Minus | 10964-11084, 11674-11817 |
| 401890 | 8516144 | Plus | 148955-149396, 149569-150002 |
| 401908 | 8698760 | Minus | 126888-127024 |
| 401913 | 9369520 | Minus | 33753-33904 |
| 401927 | 3873185 | Minus | 112000-112137 |
| 402025 | 7547159 | Plus | 173835-173998 |
| 402039 | 7770432 | Plus | 560-1294 |
| 402049 | 8072512 | Plus | 100065-100419 |
| 402085 | 7249154 | Plus | 90533-90687, 94949-95158 |
| 402241 | 7690131 | Minus | 125073-125206, 130996-131125 |
| 402294 | 2282012 | Minus | 2575-3000 |
| 402305 | 7328724 | Plus | 40832-41362 |
| 402366 | 9454515 | Plus | 195808-196863 |
| 402551 | 9856793 | Minus | 37346-37633 |
| 402654 | 8076879 | Plus | 44058-44803 |
| 402685 | 8318556 | Plus | 58962-59294 |
| 402762 | 9230904 | Minus | 123298-124035 |
| 402797 | 3421043 | Minus | 15758-15930 |
| 402833 | 8918545 | Plus | 26987-27778 |
| 402901 | 8894222 | Minus | 175426-175667 |
| 402948 | 9368458 | Minus | 143456-143626, 143808-143935 |
| 403066 | 8954202 | Plus | 158189-158433 |
| 403072 | 8954241 | Plus | 141829-142006 |
| 403146 | 9799812 | Plus | 162877-163118 |
| 403197 | 9930749 | Plus | 79990-80237 |
| 403214 | 7630945 | Minus | 76723-77027, 79317-79484 |
| 403217 | 7630969 | Plus | 54089-54163, 55427-55623 |
| 403290 | 8083176 | Plus | 19288-20076 |
| 403291 | 7230870 | Plus | 95177-95435 |
| 403294 | 8096496 | Plus | 41565-41881 |
| 403315 | 8247953 | Minus | 125117-125287 |
| 403332 | 8568139 | Minus | 31409-31674 |
| 403344 | 8569726 | Plus | 70823-70990 |
| 403362 | 8571772 | Plus | 64099-64260 |
| 403371 | 9087278 | Plus | 105655-106050 |
| 403391 | 9438337 | Plus | 42410-42544, 83317-83540, 86840-86922, 87970-88110 |
| 403488 | 9966615 | Minus | 12450-12753 |
| 403536 | 8076924 | Plus | 34972-35182 |
| 403779 | 8018040 | Minus | 95602-95969 |
| 403859 | 7708954 | Plus | 113738-113858 |
| 403871 | 7709262 | Plus | 104545-104757 |
| 403903 | 7710671 | Minus | 101165-102597 |
| 403917 | 7710849 | Plus | 109718-109847, 109927-110202 |
| 403978 | 8576014 | Plus | 97326-97808 |
| 404031 | 7671252 | Plus | 171477-172316 |
| 404167 | 9926594 | Minus | 77030-77280 |
| 404220 | 6706820 | Plus | 46107-46439 |
| 404286 | 2326514 | Plus | 51086-51301 |
| 404418 | 7382420 | Minus | 153339-153481, 155099-155294 |
| 404427 | 7407959 | Plus | 127170-127358 |
| 404429 | 7407979 | Plus | 31352-31498 |
| 404440 | 7528051 | Plus | 80430-81581 |
| 404495 | 8151634 | Minus | 59449-60477 |
| 404580 | 6539738 | Minus | 240588-241589 |
| 404606 | 9212936 | Minus | 22310-23269 |
| 404730 | 8389582 | Plus | 119832-120016, 124110-124275 |
| 404917 | 7341851 | Plus | 49330-49498 |
| 405033 | 7107731 | Minus | 142358-142546 |
| 405137 | 8570507 | Plus | 158969-159423 |
| 405146 | 9438278 | Minus | 102529-102633 |
| 405158 | 9966252 | Plus | 42873-43056, 43815-43949 |
| 405187 | 7229826 | Plus | 117025-117170, 118567-118736 |
| 405223 | 7239614 | Plus | 106184-106313 |
| 405340 | 6094635 | Plus | 49644-49760 |
| 405494 | 8050952 | Minus | 70284-70518 |
| 405551 | 1552506 | Plus | 12525-12997 |
| 405654 | 4895155 | Minus | 53624-53759 |
| 405667 | 4726099 | Plus | 5798-5914 |
| 405673 | 4589984 | Plus | 50700-50842 |
| 405704 | 4204244 | Plus | 138842-139051 |
| 405723 | 9801668 | Plus | 114896-115831 |
| 405727 | 9838331 | Minus | 78865-79664 |
| 405760 | 6066938 | Minus | 37424-38045 |
| 405779 | 7280331 | Minus | 33048-33856 |
| 405944 | 7883702 | Minus | 5143-5684 |
| 406002 | 8247797 | Minus | 154007-154579 |
| 406016 | 8272661 | Plus | 41341-41940 |
| 406097 | 7107918 | Minus | 36698-37269 |
| 406104 | 9124028 | Plus | 35309-35977 |
| 406156 | 7144867 | Plus | 379-597 |
| 406207 | 5923650 | Minus | 162607-162800 |
| 406300 | 6479046 | Minus | 19234-19401 |
| 406308 | 9211532 | Plus | 358408-358651 |
| 406314 | 9211609 | Minus | 12899-13011, 18022-18136 |
| 406317 | 9211652 | Plus | 108018-108410 |
| 406432 | 9256504 | Plus | 3804-3930, 4026-4120, 4929-5109 |
| 406490 | 7711309 | Minus | 80295-80480 |
| 406584 | 3983530 | Minus | 3989-4497 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 9A
Genes predictive of no bladder cancer progression

| Pkey | ExAccn | UnigeneID | Unigene Title | R1 | R2 |
|---|---|---|---|---|---|
| 408000 | L11690 | Hs.198689 | bullous pemphigoid antigen 1 (230/240 kD) | 4.64 | 5.88 |
| 412129 | M21984 | Hs.73454 | troponin T3, skeletal, fast | 4.54 | 5.10 |
| 459290 | NM_001546 | Hs.34853 | inhibitor of DNA binding 4, dominant neg | 4.37 | 1.63 |
| 400844 | | | NM_003105*: Homo sapiens sortilin-related | 3.69 | 5.90 |
| 419555 | AA244416 | | gb: nc07d11.s1 NCI_CGAP_Pr1 Homo sapiens | 3.61 | 2.03 |
| 414522 | AW518944 | Hs.76325 | step II splicing factor SLU7 | 3.60 | 1.00 |
| 440509 | BE410132 | Hs.134202 | ESTs, Weakly similar to T17279 hypotheti | 3.58 | 1.04 |
| 445182 | AW189787 | | ESTs | 3.57 | 2.70 |
| 407151 | H25836 | Hs.301527 | ESTs, Moderately similar to unknown [H. s | 3.56 | 1.48 |
| 421314 | BE440002 | Hs.180324 | Homo sapiens, clone IMAGE: 4183312, mRNA, | 3.28 | 3.25 |
| 429663 | M68874 | Hs.211587 | phospholipase A2, group IVA (cytosolic, | 3.25 | 2.50 |
| 430702 | U56979 | Hs.278568 | H factor 1 (complement) | 3.20 | 2.70 |
| 412420 | AL035668 | Hs.73853 | bone morphogenetic protein 2 | 3.20 | 2.30 |
| 420729 | AW964897 | Hs.290825 | ESTs | 3.20 | 1.53 |
| 433376 | AI249361 | Hs.74122 | caspase 4, apoptosis-related cysteine pr | 3.00 | 4.10 |
| 420028 | AB014680 | Hs.8786 | carbohydrate (N-acetylglucosamine-6-O) s | 2.94 | 2.78 |
| 407881 | AW072003 | Hs.40968 | heparan sulfate (glucosamine) 3-O-sulfot | 2.93 | 1.43 |
| 426283 | NM_003937 | Hs.169139 | kynureninase (L-kynurenine hydrolase) | 2.93 | 1.33 |
| 428030 | AI915228 | Hs.11493 | Homo sapiens cDNA FLJ13536 fis, clone PL | 2.92 | 2.47 |
| 419713 | AW968058 | Hs.92381 | nudix (nucleoside diphosphate linked moi | 2.89 | 3.33 |
| 414407 | AA147026 | Hs.76704 | ESTs | 2.87 | 2.87 |
| 450779 | AW204145 | Hs.156044 | ESTs | 2.78 | 1.86 |
| 411243 | AB039886 | Hs.69319 | CA11 | 2.73 | 1.00 |
| 417878 | U90916 | Hs.82845 | Homo sapiens cDNA: FLJ21930 fis, clone H | 2.68 | 2.32 |
| 441619 | NM_014056 | Hs.7917 | DKFZP564K247 protein | 2.67 | 2.98 |
| 446619 | AU076643 | Hs.313 | secreted phosphoprotein 1 (osteopontin, | 2.66 | 1.00 |
| 440006 | AK000517 | Hs.6844 | hypothetical protein FLJ20510 | 2.64 | 1.77 |
| 426252 | BE176980 | Hs.28917 | ESTs | 2.63 | 7.30 |
| 424008 | R02740 | Hs.137555 | putative chemokine receptor; GTP-binding | 2.60 | 2.53 |
| 429429 | AA829725 | Hs.334437 | hypothetical protein MGC4248 | 2.59 | 3.34 |
| 427450 | AB014526 | Hs.178121 | KIAA0626 gene product | 2.57 | 2.28 |
| 420180 | AI004035 | Hs.25191 | ESTs | 2.56 | 1.68 |
| 434061 | AW024973 | Hs.283675 | NPD009 protein | 2.54 | 2.10 |
| 422070 | AF149785 | Hs.111126 | pituitary tumor-transforming 1 interacti | 2.54 | 3.25 |
| 419355 | AA428520 | Hs.90061 | progesterone binding protein | 2.53 | 3.63 |
| 446215 | AW821329 | Hs.14368 | SH3 domain binding glutamic acid-rich pr | 2.52 | 4.38 |
| 432442 | AI672516 | Hs.178485 | ESTs, Weakly similar to S65657 alpha-1C- | 2.50 | 4.60 |
| 447887 | AA114050 | Hs.19949 | caspase 8, apoptosis-related cysteine pr | 2.49 | 2.23 |
| 401155 | | | Target Exon | 2.48 | 2.30 |
| 404530 | | | Target Exon | 2.48 | 1.00 |
| 446006 | NM_004403 | Hs.13530 | deafness, autosomal dominant 5 | 2.48 | 2.35 |
| 436476 | AA326108 | Hs.33829 | bHLH protein DEC2 | 2.47 | 2.88 |
| 446535 | AF257175 | Hs.15250 | peroxisomal D3, D2-enoyl-CoA isomerase | 2.46 | 2.19 |
| 408636 | BE294925 | Hs.46680 | CGI-12 protein | 2.45 | 1.60 |
| 420962 | NM_005904 | Hs.100602 | MAD (mothers against decapentaplegic, Dr | 2.44 | 3.75 |
| 427008 | Z45258 | Hs.286013 | short coiled-coil protein | 2.42 | 3.40 |
| 459711 | BE386801 | Hs.21858 | trinucleotide repeat containing 3 | 2.40 | 2.78 |
| 407910 | AA650274 | Hs.41296 | fibronectin leucine rich transmembrane p | 2.40 | 1.00 |
| 410337 | M83822 | Hs.62354 | cell division cycle 4-like | 2.39 | 3.88 |
| 435029 | AF167706 | Hs.19280 | cysteine-rich motor neuron 1 | 2.39 | 3.23 |
| 437181 | AI306615 | Hs.125343 | ESTs, Weakly similar to KIAA0758 protein | 2.39 | 1.00 |
| 410968 | AA199907 | Hs.67397 | homeo bus A1 | 2.38 | 1.33 |
| 422511 | AU076442 | Hs.117938 | collagen, type XVII, alpha 1 | 2.38 | 6.40 |
| 450775 | AA902384 | Hs.73853 | bone morphogenetic protein 2 | 2.38 | 2.71 |
| 442433 | BE243044 | Hs.8309 | KIAA0747 protein | 2.37 | 3.68 |
| 454000 | AA040620 | Hs.5672 | hypothetical protein AF140225 | 2.36 | 1.14 |
| 447701 | BE619526 | Hs.255527 | hypothetical protein MGC14128 | 2.36 | 2.02 |
| 427985 | AI770170 | Hs.29643 | Homo sapiens cDNA FLJ13103 fis, clone NT | 2.36 | 2.18 |
| 442257 | AW503831 | Hs.323370 | Human EST clone 25267 mariner transposon | 2.35 | 3.53 |
| 454070 | N79110 | Hs.21276 | collagen, type IV, alpha 3 (Goodpasture | 2.35 | 3.60 |
| 418452 | BE379749 | Hs.85201 | C-type (calcium dependent, carbohydrate- | 2.35 | 2.48 |
| 421218 | NM_000499 | Hs.72912 | cytochrome P450, subfamily I (aromatic c | 2.35 | 1.00 |
| 407793 | AW080879 | Hs.236572 | gb: xc38g04.x1 NCI_CGAP_Co20 Homo sapiens | 2.35 | 1.21 |
| 442061 | AA774284 | Hs.285728 | abl-interactor 12 (SH3-containing protei | 2.34 | 3.03 |
| 402845 | | | ENSP00000246267: KIAA0444 PROTEIN (FRAGME | 2.34 | 1.52 |
| 411407 | R00903 | Hs.169793 | ribosomal protein L32 | 2.34 | 0.77 |
| 418506 | AA084248 | Hs.85339 | G protein-coupled receptor 39 | 2.34 | 0.72 |
| 424637 | NM_015057 | Hs.151411 | KIAA0916 protein | 2.32 | 2.55 |
| 413804 | T64682 | | gb: yc48b02.r1 Stratagene liver (937224) | 2.32 | 1.46 |
| 411060 | NM_006074 | Hs.318501 | Homo sapiens mRNA full length insert cDN | 2.32 | 2.90 |
| 430028 | BE564110 | Hs.227750 | Target CAT | 2.32 | 2.28 |
| 417720 | AA205625 | Hs.208067 | ESTs | 2.32 | 2.09 |
| 436396 | AI683487 | Hs.152213 | wingless-type MMTV integration site fami | 2.31 | 1.13 |
| 454219 | X75042 | Hs.44313 | v-rel avian reticuloendotheliosis viral | 2.30 | 4.38 |
| 444745 | AF117754 | Hs.11861 | thyroid hormone receptor-associated prot | 2.30 | 1.86 |
| 408179 | AL042465 | Hs.43445 | poly(A)-specific ribonuclease (deadenyla | 2.29 | 2.19 |
| 442679 | R53718 | Hs.107882 | hypothetical protein FLJ10659 | 2.29 | 2.79 |
| 458949 | AW291777 | Hs.346137 | ESTs, Weakly similar to T08599 probable | 2.28 | 1.85 |
| 407191 | AA608751 | | gb: ae56h07.s1 Stratagene lung carcinoma | 2.27 | 2.42 |
| 448367 | AI955411 | Hs.94109 | Homo sapiens cDNA FLJ13634 fis, clone PL | 2.27 | 1.18 |
| 405155 | | | Target Exon | 2.26 | 1.94 |
| 445594 | AW058463 | Hs.12940 | zinc-fingers and homeoboxes 1 | 2.26 | 1.55 |
| 417458 | NM_005655 | Hs.82173 | TGFB inducible early growth response | 2.25 | 1.95 |
| 430315 | NM_004293 | Hs.239147 | guanine deaminase | 2.24 | 1.84 |
| 411945 | AL033527 | Hs.92137 | v-myc avian myelocytomatosis viral oncog | 2.24 | 2.73 |
| 408937 | AA210734 | Hs.291386 | ESTs | 2.24 | 3.18 |
| 431474 | AL133990 | Hs.190642 | CEGP1 protein | 2.23 | 1.00 |
| 434094 | AA305599 | Hs.238205 | hypothetical protein PRO2013 | 2.22 | 4.08 |
| 420997 | AK001214 | Hs.100914 | hypothetical protein FLJ10352 | 2.22 | 2.15 |
| 420164 | AW339037 | Hs.24908 | ESTs | 2.22 | 2.16 |
| 414099 | U11313 | Hs.75760 | sterol carrier protein 2 | 2.21 | 4.05 |
| 424800 | AL035588 | Hs.153203 | MyoD family inhibitor | 2.21 | 3.53 |
| 459005 | AA447679 | Hs.144558 | ESTs, Weakly similar to ALU1_HUMAN ALU S | 2.21 | 2.90 |
| 416290 | NM_000016 | Hs.79158 | acyl-Coenzyme A dehydrogenase, C-4 to C- | 2.20 | 4.00 |
| 439208 | AK000299 | Hs.180952 | dynactin 4(p62) | 2.20 | 1.88 |
| 401563 | | | C15001262: gi\|7304981\|ref\|NP_038528.1\| ca | 2.20 | 1.77 |
| 404687 | | | C9000375*: gi\|11994617\|dbj\|BAB02754.1\| (A | 2.19 | 2.60 |
| 443303 | U67319 | Hs.9216 | caspase 7, apoptosis-related cysteine pr | 2.19 | 2.08 |
| 439866 | AA280717 | Hs.6727 | Ras-GTPase activating protein SH3 domain | 2.19 | 2.21 |
| 400835 | AW853954 | | chromosome 2 open reading frame 2 | 2.18 | 3.00 |
| 456855 | AF035528 | Hs.153863 | MAD (mothers against decapentaplegic, Dr | 2.18 | 2.45 |
| 431689 | AA305688 | Hs.267695 | UDP-Gal: betaGlcNAc beta 1,3-galactosyltr | 2.17 | 1.83 |
| 418026 | BE379727 | Hs.83213 | fatty acid binding protein 4, adipocyte | 2.17 | 2.67 |
| 451131 | AI267586 | Hs.268012 | fatty-acid-Coenzyme A ligase, long-chain | 2.17 | 1.71 |
| 408038 | Y14443 | | zinc finger protein 200 | 2.17 | 1.71 |
| 434078 | AW880709 | Hs.283683 | chromosome 8 open reading frame 4 | 2.17 | 1.00 |
| 441623 | AA315805 | | desmoglein 2 | 2.17 | 1.81 |
| 459244 | AW503990 | Hs.142442 | HP1-BP74 | 2.17 | 4.03 |
| 424720 | M89907 | Hs.152292 | SWI/SNF related, matrix associated, acti | 2.17 | 2.93 |
| 404204 | | | ENSP00000252204*: Zinc finger protein 165 | 2.17 | 1.02 |
| 453987 | AA323750 | Hs.235026 | Homo sapiens, clone IMAGE: 4247529, mRNA, | 2.16 | 2.85 |
| 411400 | AA311919 | Hs.69851 | nucleolar protein family A, member 1 (H/ | 2.16 | 3.60 |
| 454949 | AW847318 | Hs.290131 | KIAA1819 protein | 2.16 | 1.96 |
| 409223 | AA312572 | Hs.6241 | phosphoinositide-3-kinase, regulatory su | 2.16 | 1.48 |
| 418030 | BE207573 | Hs.83321 | neuromedin B | 2.16 | 2.07 |
| 433364 | AI075407 | Hs.296083 | ESTs, Moderately similar to I54374 gene | 2.16 | 2.32 |
| 459511 | AI142379 | | gb: qg64c01.r1 Soares_testis_NHT Homo sap | 2.16 | 1.85 |
| 437559 | AI678033 | Hs.121476 | ESTs | 2.15 | 1.43 |
| 418827 | BE327311 | Hs.47166 | HT021 | 2.15 | 3.84 |
| 417470 | AF112219 | Hs.82193 | esterase D/formylglutathione hydrolase | 2.15 | 1.74 |
| 421012 | X53281 | Hs.101025 | basic transcription factor 3 | 2.15 | 1.26 |
| 448772 | AW390822 | Hs.301528 | L-kynurenine/alpha-aminoadipate aminotra | 2.15 | 4.05 |
| 439601 | AB029032 | Hs.6606 | KIAA1109 protein | 2.15 | 2.15 |
| 434417 | AL110157 | Hs.3843 | Home sapiens mRNA; cDNA DKFZp586F2224 (f | 2.15 | 1.45 |
| 424865 | AF011333 | Hs.153563 | lymphocyte antigen 75 | 2.15 | 2.63 |
| 400752 | | | NM_003105*: Homo sapiens sortilin-related | 2.14 | 2.67 |
| 438916 | AW188464 | Hs.101515 | ESTs | 2.14 | 2.38 |
| 430024 | AI808780 | Hs.227730 | integrin, alpha 6 | 2.14 | 2.00 |
| 409345 | AI949109 | | hypothetical protein FLJ20783 | 2.14 | 1.40 |
| 421939 | BE169531 | Hs.109727 | TAK1-binding protein 2; KIAA0733 protein | 2.13 | 1.58 |
| 442315 | AA173992 | Hs.7956 | ESTs, Moderately similar to ZN91_HUMAN Z | 2.13 | 2.67 |
| 419591 | AF090900 | Hs.91393 | Homo sapiens cDNA: FUJ21887 fis, clone H | 2.13 | 2.00 |
| 458025 | AI275406 | Hs.32450 | gb: qI63c10.x1 Soares_NhHMPu_S1 Homo sapi | 2.12 | 0.89 |
| 428582 | BE336699 | Hs.185055 | BENE protein | 2.12 | 2.65 |
| 422749 | W01076 | Hs.278573 | CD59 antigen p18-20 (antigen identified | 2.12 | 2.73 |
| 433091 | Y12642 | Hs.3185 | lymphocyte antigen 6 complex, locus D | 2.11 | 0.91 |
| 456421 | AL157485 | Hs.91973 | hypothetical protein | 2.11 | 2.51 |
| 421508 | NM_004833 | Hs.105115 | absent in melanoma 2 | 2.11 | 3.13 |
| 402760 | | | NM_021797*: Homo sapiens eosinophil chemo | 2.09 | 1.79 |
| 406274 | | | Target Exon | 2.09 | 1.60 |
| 406897 | M57417 | | gb: Home sapiens mucin (mucin) mRNA, part | 2.09 | 1.00 |
| 409632 | W74001 | Hs.55279 | serine (or cysteine) proteinase inhibito | 2.09 | 2.92 |
| 445320 | AA503887 | Hs.167011 | Home sapiens cDNA: FLJ21362 fis, clone C | 2.09 | 3.20 |
| 442271 | AF000652 | Hs.8180 | syndecan binding protein (syntenin) | 2.09 | 1.90 |
| 428336 | AA503115 | Hs.183752 | microseminoprotein, beta- | 2.08 | 1.15 |
| 405165 | | | ENSP00000238974*: Homeobox protein NKX2-3 | 2.07 | 2.83 |
| 416999 | AW195747 | Hs.21122 | hypothetical protein FLJ11830 similar to | 2.07 | 3.71 |
| 453865 | AA307279 | Hs.35947 | methyl-CpG binding domain protein 4 | 2.07 | 1.71 |
| 439924 | AI985897 | Hs.125293 | ESTs | 2.07 | 1.00 |
| 439004 | AW979062 | | gb: EST391172 MAGE resequences, MAGP Homo | 2.07 | 2.13 |
| 407955 | BE536739 | Hs.109909 | ESTs | 2.06 | 1.91 |
| 412998 | BE046254 | | gb: hn38g09.x2 NCI_CGAP_RDF2 Homo sapiens | 2.06 | 2.58 |
| 414013 | AA766605 | Hs.47099 | hypothetical protein FLJ21212 | 2.05 | 5.00 |
| 415249 | R40515 | Hs.21248 | ESTs | 2.05 | 2.18 |
| 427332 | R09418 | Hs.261101 | ESTs, Weakly similar to I38022 hypotheti | 2.05 | 3.35 |
| 426521 | AF161445 | Hs.170219 | hypothetical protein | 2.05 | 1.00 |
| 431211 | M86849 | Hs.323733 | gap junction protein, beta 2, 26 kD (conn | 2.05 | 6.03 |
| 423851 | R39505 | Hs.133342 | Homo sapiens clone 24566 mRNA sequence | 2.05 | 1.88 |
| 410028 | AW576454 | Hs.346502 | ESTs | 2.04 | 1.95 |
| 406575 | | | Target Exon | 2.04 | 1.56 |
| 457148 | AF091035 | Hs.184627 | KIAA0118 protein | 2.04 | 3.11 |
| 449924 | W30681 | Hs.146233 | Homo sapiens cDNA: FLJ22130 fis, clone H | 2.04 | 2.42 |
| 429837 | NM_003896 | Hs.225939 | sialyltransferase 9 (CMP-NeuAc: lactosylc | 2.04 | 1.97 |
| 440675 | AW005054 | Hs.47883 | ESTs, Weakly similar to KCC1_HUMAN CALCI | 2.04 | 2.06 |
| 411988 | AA455459 | Hs.164480 | ESTs, Weakly similar to T50609 hypotheti | 2.04 | 2.65 |
| 433293 | AF007835 | Hs.32417 | hypothetical protein MGC4309 | 2.04 | 2.35 |
| 446187 | AK001241 | Hs.14229 | hypothetical protein FLJ10379 | 2.04 | 2.03 |
| 420838 | AW118210 | Hs.42321 | ESTs | 2.03 | 1.00 |
| 445481 | AW661846 | Hs.346630 | ESTs | 2.03 | 2.49 |
| 448175 | BE296174 | Hs.225160 | hypothetical protein FLJ13102 | 2.03 | 2.25 |
| 410600 | AW575742 | | ESTs, Moderately similar to S65657 alpha | 2.02 | 2.10 |
| 401177 | | | Target Exon | 2.02 | 2.59 |
| 448474 | AI792014 | Hs.13809 | hypothetical protein FLJ10648 | 2.02 | 4.23 |
| 434782 | NM_005032 | Hs.4114 | plastin 3 (T isoform) | 2.02 | 1.48 |
| 424125 | M31669 | Hs.1735 | inhibin, beta B (activin AB beta polypep | 2.02 | 2.93 |
| 424241 | AW995948 | Hs.182339 | Homo sapiens pyruvate dehydrogenase kina | 2.02 | 2.63 |
| 424673 | AA345051 | Hs.294092 | ESTs, Weakly similar to I38022 hypotheti | 2.02 | 3.43 |
| 414721 | X90392 | Hs.77091 | ribosomal protein L10 | 2.02 | 1.89 |
| 429869 | AI907018 | Hs.15977 | Target CAT | 2.02 | 1.47 |
| 439177 | AW820275 | Hs.76611 | ESTs, Weakly similar to I38022 hypotheti | 2.01 | 1.94 |
| 437175 | AW968078 | Hs.87773 | protein kinase, cAMP-dependent, catalyti | 2.01 | 1.64 |
| 452046 | AB018345 | Hs.27657 | KIAA0802 protein | 2.01 | 4.31 |
| 417615 | BE548641 | Hs.82314 | hypoxanthine phosphoribosyltransferase 1 | 2.01 | 6.75 |
| 420337 | AW295840 | Hs.14555 | Homo sapiens cDNA: FLJ21513 fis, clone C | 2.00 | 2.75 |
| 408232 | AL137269 | Hs.43899 | Homo sapiens mRNA; cDNA DKFZp434C1714 (f | 2.00 | 2.02 |
| 408409 | AW838181 | Hs.278337 | Homo sapiens cDNA FLJ11537 fis, clone HE | 2.00 | 1.95 |
| 433256 | AW604447 | Hs.339408 | ESTs, Weakly similar to S26689 hypotheti | 2.00 | 0.91 |
| 426969 | AI936504 | Hs.2083 | CDC-like kinase 1 | 2.00 | 3.60 |
| 442053 | R35343 | Hs.24968 | Human DNA sequence from clone RP1-233G16 | 2.00 | 1.95 |
| 444916 | AB028956 | Hs.12144 | KIAA1033 protein | 2.00 | 1.23 |
| 452286 | AI358570 | Hs.123933 | ESTs, Weakly similar to ZN91_HUMAN ZINC | 2.00 | 5.30 |
| 414906 | AA157911 | Hs.72200 | ESTs | 1.99 | 1.22 |
| 414176 | BE140638 | Hs.75794 | endothelial differentiation, lysophospha | 1.99 | 3.83 |
| 414557 | AA340111 | Hs.100009 | acyl-Coenzyme A oxidase 1, palmitoyl | 1.99 | 2.31 |
| 452846 | AA082160 | Hs.63368 | ESTs, Weakly similar to TRHY_HUMAN TRICH | 1.99 | 3.43 |
| 408437 | AW957744 | Hs.278469 | lacrimal proline rich protein | 1.98 | 2.15 |
| 439205 | AF087990 | Hs.42758 | Homo sapiens, clone IMAGE: 3354845, mRNA, | 1.98 | 2.28 |
| 442506 | BE566411 | | ESTs | 1.98 | 3.95 |
| 447731 | AA373527 | Hs.19385 | CGI-58 protein | 1.98 | 2.67 |
| 410579 | AK001628 | Hs.64691 | KIAA0483 protein | 1.97 | 2.43 |
| 426716 | NM_006379 | Hs.171921 | sema domain, immunoglobulin domain (lg), | 1.97 | 2.50 |
| 456141 | AI751357 | Hs.288741 | Homo sapiens cDNA: FLJ22256 fis, clone H | 1.97 | 3.03 |
| 419576 | AK002060 | Hs.91251 | hypothetical protein FLJ11198 | 1.96 | 2.88 |
| 407241 | M34516 | | gb: Human omega light chain protein 14.1 | 1.96 | 1.09 |
| 420664 | AI681270 | Hs.99824 | BCE-1 protein | 1.96 | 1.75 |
| 448586 | AF285120 | Hs.283734 | CGI-204 protein | 1.96 | 3.28 |
| 408089 | H59799 | Hs.42644 | thioredoxin-like | 1.95 | 4.00 |
| 421100 | AW351839 | Hs.124660 | Homo sapiens cDNA: FLJ21763 fis, clone C | 1.95 | 2.12 |
| 452518 | AA280722 | Hs.24758 | ESTs, Weakly similar to I38022 hypotheti | 1.95 | 3.45 |
| 432015 | AL157504 | Hs.159115 | Homo sapiens mRNA; cDNA DKFZp586O0724 (t | 1.94 | 2.80 |
| 434263 | N34895 | Hs.44648 | ESTs | 1.94 | 4.60 |
| 409829 | M33552 | Hs.56729 | lymphocyte-specific protein 1 | 1.94 | 1.79 |
| 425593 | AA278921 | Hs.1908 | proteoglycan 1, secretory granule | 1.94 | 2.30 |
| 401835 | | | Target Exon | 1.94 | 2.27 |
| 406557 | | | C5000893: gi\|6226859\|sp\|P38525\|EFG_THEMA | 1.94 | 3.28 |
| 440062 | AI350518 | Hs.129692 | ESTs | 1.94 | 3.18 |
| 410442 | X73424 | Hs.63788 | propionyl Coenzyme A carboxylase, beta p | 1.94 | 2.70 |
| 457281 | BE253012 | Hs.153400 | ESTs, Weakly similar to ALU1_HUMAN ALU S | 1.94 | 2.60 |
| 420230 | AL034344 | Hs.284186 | forkhead box C1 | 1.93 | 2.28 |
| 452970 | NM_012238 | Hs.31176 | sirtuin (silent mating type information | 1.93 | 4.35 |
| 403728 | | | Target Exon | 1.92 | 1.70 |
| 415789 | H01581 | | gb: yj33f08.r1 Soares placenta Nb2HP Homo | 1.92 | 2.15 |
| 406759 | AA654582 | Hs.77039 | ATP synthase, H transporting, mitochondr | 1.92 | 2.10 |
| 442073 | AW973443 | Hs.8086 | RNA (guanine-7-) methyltransferase | 1.92 | 4.43 |
| 438023 | AF204883 | Hs.6048 | FEM-1 (C. elegans) homolog b | 1.92 | 4.00 |
| 445502 | AW379160 | Hs.12813 | DKFZP434J214 protein | 1.92 | 2.13 |
| 405474 | | | NM_001093*: Homo sapiens acetyl-Coenzyme | 1.92 | 2.58 |
| 430007 | NM_014892 | Hs.227602 | KIAA1116 protein | 1.92 | 3.78 |
| 439937 | AF151906 | Hs.6776 | CGI-148 protein | 1.91 | 2.32 |
| 418068 | AW971155 | Hs.293902 | ESTs, Weakly similar to ISHUSS protein d | 1.91 | 1.88 |
| 444630 | AI753230 | Hs.323562 | hypothetical protein DKFZp564K142 | 1.91 | 1.61 |
| 451184 | T87943 | Hs.173638 | transcription factor 7-like 2 (T-cell sp | 1.90 | 3.35 |
| 414715 | AA587891 | Hs.904 | amylo-1,6-glucosidase, 4-alpha-glucanotr | 1.90 | 3.55 |
| 445841 | AL080115 | Hs.13370 | DKFZP564G0222 protein | 1.90 | 1.46 |
| 425284 | AF155568 | Hs.348043 | NS1-associated protein 1 | 1.90 | 3.65 |
| 437943 | NM_016353 | Hs.5943 | rec | 1.89 | 1.73 |
| 442426 | AI373062 | Hs.332938 | hypothetical protein MGC5370 | 1.89 | 2.79 |
| 400111 | | | Eos Control | 1.89 | 3.84 |
| 437762 | T78028 | Hs.154679 | synaptotagmin I | 1.89 | 1.00 |
| 404069 | | | Target Exon | 1.89 | 2.51 |
| 434809 | AW974687 | | gb: EST386776 MAGE resequences, MAGM Homo | 1.88 | 2.35 |
| 414220 | BE298094 | Hs.323806 | gb: 601118231F1 NIH_MGC_17 Homo sapiens c | 1.88 | 1.00 |
| 422506 | R20909 | Hs.300741 | sorcin | 1.87 | 2.99 |
| 417439 | AW602154 | Hs.82143 | E74-like factor 2 (ets domain transcript | 1.87 | 1.13 |
| 404391 | | | Target Exon | 1.87 | 3.00 |
| 420187 | AK001714 | Hs.95744 | hypothetical protein similar to ankyrin | 1.86 | 2.93 |
| 446950 | AA305800 | Hs.5672 | hypothetical protein AF140225 | 1.86 | 1.90 |
| 400634 | | | C10000818*: gi\|7661882\|ref\|NP_055697.1\| K | 1.86 | 2.80 |
| 408455 | C19034 | Hs.288613 | Homo sapiens cDNA FLJ14175 fis, clone NT | 1.86 | 1.32 |
| 422366 | T83882 | Hs.97927 | ESTs | 1.85 | 1.44 |
| 452170 | AF064801 | Hs.28285 | patched related protein translocated in | 1.85 | 2.64 |
| 430604 | AV650537 | Hs.247309 | succinate-CoA ligase, GDP-forming, beta | 1.85 | 1.81 |
| 426484 | AA379658 | Hs.272759 | KIAA1457 protein | 1.85 | 2.60 |
| 411609 | AW993680 | | gb: RC3-BN0034-290200-013-d08 BN0034 Homo | 1.85 | 2.10 |
| 431129 | AL137751 | Hs.263671 | Homo sapiens mRNA; cDNA DKFZp434I0812 (f | 1.84 | 3.70 |
| 412843 | AF007555 | Hs.74624 | protein tyrosine phosphatase, receptor t | 1.84 | 2.58 |
| 401512 | | | NM_014080: Homo sapiens dual oxidase-like | 1.84 | 1.52 |
| 415969 | H11294 | Hs.31047 | ESTs | 1.84 | 3.08 |
| 444736 | AA533491 | Hs.23317 | hypothetical protein FLJ14681 | 1.84 | 1.20 |
| 426418 | M90464 | Hs.169825 | collagen, type IV, alpha 5 (Alport syndr | 1.84 | 2.35 |
| 416968 | AA412686 | Hs.97955 | ESTs | 1.84 | 2.18 |
| 442961 | BE614474 | Hs.289074 | F-box only protein 22 | 1.84 | 2.18 |
| 418650 | BE386750 | Hs.86978 | prolyl endopeptidase | 1.84 | 1.98 |
| 420923 | AF097021 | Hs.273321 | differentially expressed in hematopoieti | 1.84 | 1.00 |
| 432834 | F06459 | Hs.289113 | cytochrome b5 reductase 1 (B5R.1) | 1.83 | 3.93 |
| 442485 | BE092285 | Hs.29724 | hypothetical protein FLJ13187 | 1.83 | 3.10 |
| 427699 | AW965076 | Hs.180378 | hypothetical protein 669 | 1.83 | 3.03 |
| 447387 | AI268331 | Hs.102237 | tubby super-family protein | 1.83 | 1.78 |
| 418663 | AK00100 | Hs.41690 | desmocollin 3 | 1.82 | 1.53 |
| 419733 | AW362955 | Hs.224961 | Homo sapiens cDNA FLJ14415 fis, clone HE | 1.82 | 1.00 |
| 409267 | NM_012453 | Hs.52515 | transducin (beta)-like 2 | 1.81 | 1.57 |
| 413341 | H78472 | Hs.191325 | ESTs, Weakly similar to T18967 hypotheti | 1.81 | 2.05 |
| 423810 | AL132665 | Hs.132955 | BCL2/adenovirus E1B 19 kD-interacting pro | 1.81 | 1.98 |
| 416274 | AW160404 | Hs.79126 | guanine nucleotide binding protein 10 | 1.80 | 1.91 |
| 400843 | | | NM_003105*: Homo sapiens sortilin-related | 1.80 | 4.88 |
| 442187 | N23532 | Hs.288963 | Homo sapiens cDNA: FLJ23034 fis, clone L | 1.80 | 2.61 |
| 458285 | AW296984 | Hs.255595 | ESTs, Weakly similar to A46302 PTB-assoc | 1.80 | 2.33 |
| 413753 | U17760 | Hs.75517 | laminin, beta 3 (nicein (125 kD), kalinin | 1.80 | 3.17 |
| 428004 | AA449563 | Hs.151393 | glutamate-cysteine ligase, catalytic sub | 1.80 | 1.00 |
| 401613 | | | Target Exon | 1.79 | 2.66 |
| 407173 | T64349 | | gb: yc10d08.s1 Stratagene lung (937210) H | 1.79 | 2.30 |
| 443145 | AI049671 | Hs.307763 | EST, Weakly similar to I38022 hypothetic | 1.79 | 2.00 |
| 418596 | AW976721 | Hs.293327 | ESTs | 1.79 | 3.92 |
| 437374 | AL359571 | Hs.44054 | ninein (GSK3B interacting protein) | 1.79 | 1.24 |
| 439569 | AW602166 | Hs.222399 | CEGP1 protein | 1.79 | 2.39 |
| 430677 | Z26317 | Hs.94560 | desmoglein 2 | 1.78 | 2.02 |
| 436749 | AA584890 | Hs.5302 | lectin, galactoside-binding, soluble, 4 | 1.78 | 0.96 |
| 453016 | AW295466 | Hs.232051 | ESTs, Weakly similar to dJ403A15.3 [H. sa | 1.78 | 2.60 |
| 426885 | AA393130 | Hs.193894 | ESTs, Weakly similar to A47582 B-cell gr | 1.78 | 2.47 |
| 452848 | AI417193 | Hs.288912 | hypothetical protein FLJ22604 | 1.78 | 2.17 |
| 412560 | R24601 | | CCR4-NOT transcription complex, subunit | 1.78 | 3.13 |
| 411821 | BE299339 | Hs.72249 | three-PDZ containing protein similar to | 1.78 | 1.55 |
| 428788 | AF082283 | Hs.193516 | B-cell CLL/lymphoma 10 | 1.78 | 2.36 |
| 443963 | AA878183 | Hs.17448 | Homo sapiens cDNA FLJ13618 fis, clone PL | 1.78 | 2.20 |
| 435479 | AF197137 | Hs.259737 | ATP synthase, H transporting, mitochondr | 1.78 | 2.03 |
| 413073 | AL038165 | Hs.75187 | translocase of outer mitochondrial membr | 1.77 | 2.29 |
| 442473 | W27992 | | gb: 43d9 Haman retina cDNA randomly prime | 1.77 | 2.93 |
| 418060 | AA211589 | Hs.208047 | ESTs | 1.77 | 4.19 |
| 400773 | | | NM_003105*: Homo sapiens sortilin-related | 1.77 | 1.76 |
| 400175 | | | Eos Control | 1.77 | 2.04 |
| 421501 | M29971 | Hs.1384 | O-6-methylguanine-DNA methyltransferase | 1.77 | 2.32 |
| 451234 | AI914901 | Hs.24052 | ESTs, Weakly similar to I38022 hypotheti | 1.77 | 2.43 |
| 423332 | Al091466 | Hs.127241 | sorting nexin 7 | 1.76 | 1.82 |
| 423960 | AA164516 | Hs.136309 | SH3-containing protein SH3GLB1 | 1.76 | 2.00 |
| 450489 | AI697990 | Hs.346002 | ESTs | 1.76 | 3.15 |
| 457265 | AB023212 | Hs.225967 | KIAA0995 protein | 1.76 | 2.37 |
| 413076 | U10564 | Hs.75188 | wee1 (S. pombe) homolog | 1.75 | 2.18 |
| 421948 | L42583 | Hs.334309 | keratin 6A | 1.75 | 1.00 |
| 453578 | R06875 | Hs.81810 | ESTs | 1.75 | 3.10 |
| 412430 | AW675064 | Hs.73875 | fumarylacetoacetate hydrolase (fumarylac | 1.75 | 2.14 |
| 439396 | BE562958 | Hs.74346 | hypothetical protein MGC14353 | 1.75 | 1.78 |
| 431448 | AL137517 | Hs.306201 | hypothetical protein DKFZp564O1278 | 1.75 | 2.36 |
| 449538 | AI559444 | Hs.104679 | ESTs | 1.75 | 3.07 |
| 453146 | AI338952 | Hs.32194 | ESTs | 1.74 | 2.82 |
| 426122 | NM_006925 | Hs.166975 | Splicing factor, arginine/serine-rich 5 | 1.74 | 2.88 |
| 408989 | AW361666 | Hs.49500 | KIAA0746 protein | 1.74 | 2.07 |
| 441715 | AI929453 | Hs.342655 | Homo sapiens cDNA FLJ13289 fis, clone OV | 1.74 | 2.06 |
| 412718 | X79204 | Hs.74520 | spinocerebellar ataxia 1 (olivopontocere | 1.74 | 2.46 |
| 450798 | AW167780 | Hs.50438 | ESTs | 1.74 | 2.02 |
| 445537 | AJ245671 | Hs.12844 | EGF-like-domain, multiple 6 | 1.73 | 2.58 |
| 400190 | | | Eos Control | 1.73 | 2.40 |
| 416309 | R84694 | Hs.79194 | cAMP responsive element binding protein | 1.73 | 1.48 |
| 410219 | T98226 | Hs.171952 | occludin | 1.73 | 2.75 |
| 419814 | AW402478 | Hs.93213 | BCL2-antagonist/killer 1 | 1.73 | 2.70 |
| 448625 | AW970786 | Hs.178470 | hypothetical protein FLJ22662 | 1.73 | 2.07 |
| 422387 | AA309996 | Hs.148656 | ESTs, Weakly similar to T12453 hypotheti | 1.73 | 2.02 |
| 417386 | AL037228 | Hs.82043 | D123 gene product | 1.73 | 2.44 |
| 405812 | | | Target Exon | 1.72 | 2.94 |
| 436270 | C03769 | Hs.339669 | Homo sapiens, clone IMAGE: 3947554, mRNA, | 1.72 | 2.85 |
| 409855 | AW502461 | | gb: UI-HF-BR0p-ajv-b-08-0-UI.r1 NIH_MGC_5 | 1.72 | 2.63 |
| 411442 | N25956 | Hs.101810 | Homo sapiens cDNA FLJ14232 fis, clone NT | 1.72 | 1.88 |
| 400846 | | | sortilin-related receptor, L(DLR class) | 1.72 | 1.63 |
| 401660 | | | Target Exon | 1.72 | 2.63 |
| 402190 | | | C19000835*:gi\|10946730\|ref\|NP_067362.1\| | 1.72 | 3.33 |
| 439191 | AA281177 | Hs.41182 | Homo sapiens DC47 mRNA, complete cds | 1.71 | 2.17 |
| 410444 | W73484 | Hs.132554 | gb: zd54e04.s1 Soares_fetal_heart_NbHH19W | 1.71 | 2.70 |
| 430393 | BE185030 | Hs.241305 | estrogen-responsive B box protein | 1.71 | 1.33 |
| 446066 | AI343931 | Hs.149383 | ESTs | 1.71 | 2.32 |
| 411299 | BE409857 | Hs.69499 | hypothetical protein | 1.71 | 2.92 |
| 408246 | N55669 | Hs.333823 | mitochondrial ribosomal protein L13 | 1.71 | 2.00 |
| 454054 | AI336329 | Hs.301519 | Homo sapiens cDNA FLJ12536 fis, clone NT | 1.71 | 1.93 |
| 417381 | AF164142 | Hs.82042 | solute carrier family 23 (nucleobase tra | 1.70 | 3.70 |
| 427820 | BE222494 | Hs.180919 | inhibitor of DNA binding 2, dominant neg | 1.70 | 1.60 |
| 400750 | | | Target Exon | 1.70 | 2.82 |
| 455842 | BE145837 | | gb: MR0-HT0208-101299-202-c07 HT0208 Homo | 1.70 | 2.17 |
| 429966 | BE081342 | Hs.283037 | HSPC039 protein | 1.70 | 1.18 |
| 418444 | AI902899 | Hs.85155 | butyrate response factor 1 (EGF-response | 1.70 | 2.47 |
| 437450 | AL390154 | Hs.26954 | Homo sapiens mRNA; cDNA DKFZp762G123 (fr | 1.70 | 3.03 |
| 415738 | BE539367 | Hs.295953 | ESTs, Weakly similar to AF220049 1 uncha | 1.70 | 2.34 |
| 405245 | | | Target Exon | 1.70 | 1.99 |
| 408483 | AA464836 | Hs.291079 | ESTs, Weakly similar to T27173 hypotheti | 1.70 | 2.05 |
| 413611 | BE153275 | | gb: PM0-HT0335-180400-008-e11 HT0335 Homo | 1.70 | 2.05 |
| 410190 | AW072328 | Hs.59728 | Homo sapiens mRNA; cDNA DKFZp566C0546 (f | 1.69 | 2.20 |
| 434608 | AA805443 | Hs.179909 | hypothetical protein FLJ22995 | 1.69 | 2.36 |
| 432170 | T56887 | Hs.18282 | KIAA1134 protein | 1.69 | 1.83 |
| 448182 | AF244137 | Hs.20597 | host cell factor homolog | 1.69 | 2.11 |
| 436293 | AI601188 | Hs.120910 | ESTs | 1.69 | 2.37 |
| 448524 | AB032948 | Hs.21356 | hypothetical protein DKFZp762K2015 | 1.68 | 2.48 |
| 404231 | | | Target Exon | 1.68 | 2.50 |
| 453906 | AW444952 | Hs.257054 | ESTs | 1.68 | 2.45 |
| 437967 | BE277414 | Hs.5947 | met transforming oncogene (derived from | 1.68 | 1.00 |
| 426125 | X87241 | Hs.166994 | FAT tumor suppressor (Drosophila) homolo | 1.68 | 3.51 |
| 448813 | AF169802 | Hs.22142 | cytochrome b5 reductase b5R.2 | 1.68 | 1.69 |
| 429162 | AK001250 | Hs.197642 | hypothetical protein FLJ10388 | 1.68 | 3.13 |
| 425556 | H27225 | Hs.9444 | hypothetical protein FLJ13114 | 1.67 | 2.02 |
| 405630 | | | Target Exon | 1.67 | 3.00 |
| 421405 | AA251944 | Hs.104058 | CGI-29 protein | 1.67 | 3.25 |
| 422640 | M37984 | Hs.118845 | troponin C, slow | 1.67 | 1.23 |
| 450857 | AA629075 | Hs.190090 | ESTs | 1.67 | 2.48 |
| 451668 | Z43948 | Hs.326444 | cartilage acidic protein 1 | 1.66 | 2.55 |
| 433821 | AW182416 | | ESTs | 1.66 | 2.65 |
| 405595 | | | NM_000721*: Homo sapiens calcium channel, | 1.66 | 2.23 |
| 433892 | AI929357 | Hs.323966 | Homo sapiens clone H63 unknown mRNA | 1.66 | 1.97 |
| 443558 | AA376798 | Hs.286122 | MDS024 protein | 1.66 | 2.00 |
| 412141 | AI183838 | Hs.48938 | hypothetical protein FLJ21802 | 1.66 | 2.65 |
| 424685 | W21223 | Hs.151734 | nuclear transport factor 2 (placental pr | 1.66 | 2.88 |
| 400845 | | | NM_003105*: Homo sapiens sortilin-related | 1.66 | 1.61 |
| 447816 | NM_007233 | Hs.274329 | TP53 target gene 1 | 1.66 | 2.63 |
| 404438 | | | Target Exon | 1.66 | 2.34 |
| 451543 | AA397651 | Hs.301959 | proline synthetase co-transcribed (bacte | 1.65 | 2.08 |
| 433233 | AB040927 | Hs.301804 | KIAA1494 protein | 1.65 | 3.13 |
| 420938 | AL049698 | Hs.100469 | myeloid/lymphoid or mixed-lineage leukem | 1.65 | 1.37 |
| 435438 | H84421 | Hs.4890 | ubiquitin-conjugating enzyme E2E 3 (homo | 1.65 | 2.35 |
| 431130 | NM_006103 | Hs.2719 | HE4; epididymis-specific, whey-acidic pr | 1.65 | 1.00 |
| 433235 | AB040929 | Hs.35089 | contactin 3 (plasmacytoma associated) | 1.65 | 1.44 |
| 439632 | AW410714 | Hs.334437 | hypothetical protein MGC4248 | 1.65 | 2.35 |
| 409324 | W76202 | Hs.343812 | lipoic acid synthetase | 1.65 | 2.00 |
| 452207 | NM_014517 | Hs.28423 | upstream binding protein 1 (LBP-1a) | 1.65 | 2.33 |
| 423630 | AB011132 | Hs.129952 | KIAA0560 gene product | 1.65 | 2.13 |
| 443358 | H65417 | Hs.17757 | pleckstrin homology domain-containing, f | 1.65 | 1.63 |
| 427417 | AA341061 | Hs.177861 | CGI-110 protein | 1.64 | 1.28 |
| 450353 | AI244661 | Hs.103296 | ESTs,Weakly similar to S65657 alpha-1C- | 1.64 | 1.60 |
| 445677 | H96577 | Hs.6838 | ras homolog gene family, member E | 1.64 | 1.91 |
| 447503 | AA115496 | Hs.336898 | Homo sapiens, Similar to RlKEN cDNA 1810 | 1.64 | 2.04 |
| 431234 | AL389985 | Hs.301637 | zinc finger protein 258 | 1.64 | 1.53 |
| 418032 | AW964695 | Hs.9436 | Homo sapiens, clone MGC: 15763, mRNA, com | 1.64 | 2.05 |
| 407796 | AA195509 | Hs.39733 | postsynaptic protein CRIPT | 1.64 | 2.30 |
| 446298 | AF187813 | Hs.14637 | kidney-and liver-specific gene | 1.64 | 2.05 |
| 439578 | AW263124 | Hs.315111 | nuclear receptor co-repressor/HDAC3 comp | 1.64 | 2.26 |
| 429113 | D28235 | Hs.196384 | prostaglandin-endoperoxide synthase 2 (p | 1.64 | 2.10 |
| 433646 | AA603319 | Hs.155195 | ESTs | 1.64 | 2.05 |
| 407783 | AW996872 | Hs.172028 | a disintegrin and metalloproteinase doma | 1.64 | 1.00 |
| 419982 | AA252544 | Hs.55610 | solute carrier family 30 (zinc transport | 1.64 | 2.16 |
| 401603 | | | NM_022041*: Homo sapiens giant axonal neu | 1.64 | 2.73 |
| 431604 | AF175265 | Hs.264190 | vacuolar protein sorting 35 (yeast homol | 1.64 | 2.75 |
| 400788 | | | C6000994*: gi\|10435784\|dbj\|BAB14668.1\| (A | 1.63 | 2.04 |
| 416221 | BE513171 | Hs.79086 | mitochondrial ribosomal protein L3 | 1.63 | 2.64 |
| 422491 | AA338548 | Hs.117546 | neuronatin | 1.63 | 0.96 |
| 424737 | BE301883 | Hs.152707 | glioblastoma amplified sequence | 1.63 | 3.45 |
| 416078 | AL034349 | Hs.79005 | protein tyrosine phosphatase, receptor t | 1.63 | 1.39 |
| 403988 | | | C5001831: gi\|11056014\|ref\|NP_067651.1\| ac | 1.62 | 2.11 |
| 411486 | N85785 | Hs.181165 | eukaryotic translation elongation factor | 1.62 | 2.63 |
| 407874 | AI766311 | Hs.289047 | Home sapiens cDNA FLJ14059 fis, clone HE | 1.62 | 2.19 |
| 446700 | AW206257 | Hs.156326 | Human DNA sequence from clone RP11-145L2 | 1.62 | 3.03 |
| 438184 | AA779897 | Hs.122125 | ESTs | 1.62 | 2.79 |
| 405502 | | | C7000609*: gi\|628012\|pir∥A53933 myosin I | 1.62 | 2.55 |
| 447050 | NM_016314 | Hs.17200 | STAM-like protein containing SH3 and ITA | 1.62 | 2.48 |
| 457961 | AA772119 | Hs.270721 | ESTs, Weakly similar to I38022 hypotheti | 1.62 | 2.30 |
| 436774 | AW975810 | Hs.159054 | hypothetical protein FLJ13224 | 1.62 | 2.17 |
| 414893 | AA215295 | Hs.77578 | ubiquitin specific protease 9, X chromos | 1.62 | 2.03 |
| 458660 | AI299739 | Hs.99601 | hypothetical protein FLJ12553 | 1.62 | 2.25 |
| 405806 | | | Target Exon | 1.62 | 2.15 |
| 421205 | AL137540 | Hs.102541 | netrin 4 | 1.62 | 1.00 |
| 424012 | AW368377 | Hs.137569 | tumor protein 63 kDa with strong homolog | 1.62 | 1.74 |
| 427016 | AA397525 | Hs.191579 | ESTs | 1.61 | 2.16 |
| 458182 | AI147996 | Hs.155833 | ESTs, Weakly similar to spliceosomal pro | 1.61 | 2.74 |
| 451109 | F11875 | Hs.5534 | Homo sapiens cDNA FLJ12961 fis, clone NT | 1.61 | 2.59 |
| 414807 | AI738616 | Hs.77348 | hydroxyprostaglandin dehydrogenase 15-(N | 1.61 | 1.78 |
| 456508 | AA502764 | Hs.123469 | ESTs, Weakly similar to AF208855 1 BM-01 | 1.61 | 2.10 |
| 447532 | AK000614 | Hs.18791 | hypothetical protein FLJ20607 | 1.61 | 1.75 |
| 439944 | AA856767 | Hs.124623 | ESTs | 1.61 | 2.41 |
| 414692 | H06831 | Hs.164557 | ESTs, Moderately similar to ALUC_HUMAN ! | 1.60 | 3.05 |
| 433187 | R53995 | Hs.293381 | ESTs, Moderately similar to ALU7_HUMAN A | 1.60 | 2.63 |
| 446825 | BE266822 | Hs.344097 | filamin A, alpha (actin-binding protein- | 1.60 | 2.43 |
| 441166 | AA921738 | Hs.132473 | ESTs | 1.60 | 2.69 |
| 425571 | AJ007292 | Hs.158306 | ephrin-A2 | 1.60 | 1.49 |
| 406836 | AW514501 | Hs.156110 | immunoglobulin kappa constant | 1.60 | 1.08 |
| 432374 | W68815 | Hs.301885 | Home sapiens cDNA FLJ11346 fis, clone PL | 1.60 | 1.47 |
| 449268 | AW369278 | Hs.23412 | hypothetical protein FLJ20160 | 1.60 | 2.89 |
| 400772 | | | NM_003105*: Homo sapiens sortilin-related | 1.60 | 2.57 |
| 445733 | BE295568 | Hs.13225 | UDP-Gal: betaGlcNAc beta 1,4- galactosylt | 1.60 | 2.03 |
| 428172 | U09367 | Hs.182828 | zinc finger protein 136 (clone pHZ-20) | 1.60 | 2.68 |
| 421887 | AW161450 | Hs.109201 | CGI-86 protein | 1.59 | 1.39 |
| 418127 | BE243982 | Hs.83532 | membrane cofactor protein (CD46, trophob | 1.59 | 1.67 |
| 400297 | AI127076 | Hs.306201 | hypothetical protein DKFZp564O1278 | 1.59 | 2.19 |
| 434938 | AW500718 | Hs.8115 | Homo sapiens, clone MGC: 16169, mRNA, com | 1.59 | 2.26 |
| 417924 | AU077231 | Hs.82932 | cyclin D1 (PRAD1: parathyroid adenomatos | 1.59 | 1.76 |
| 418067 | AI127958 | Hs.83393 | cystatin E/M | 1.59 | 1.26 |
| 427127 | AW802282 | Hs.22265 | pyruvate dehydrogenase phosphatase | 1.59 | 2.25 |
| 451938 | AI354355 | Hs.16697 | down-regulator of transcription 1, TBP-b | 1.59 | 2.10 |
| 407325 | AA291180 | Hs.328476 | ESTs, Weakly similar to alternatively sp | 1.58 | 2.43 |
| 410796 | Z44547 | Hs.3731 | ESTs, Moderately similar to I38022 hypot | 1.58 | 1.26 |
| 417343 | AA197132 | Hs.231581 | myosin, heavy polypeptide 1, skeletal mu | 1.58 | 2.84 |
| 416643 | U62531 | Hs.79410 | solute carrier family 4, anion exchanger | 1.58 | 1.26 |
| 400847 | | | NM_003105*: Homo sapiens sortilin-related | 1.58 | 1.48 |
| 436760 | AW606927 | Hs.5306 | hypothetical protein DKFZp586F1122 simil | 1.57 | 1.57 |
| 433427 | AI816449 | Hs.171889 | cholinephosphotransferase 1 | 1.57 | 1.64 |
| 451986 | BE246996 | Hs.318401 | hypothetical protein DKFZp564D1378 | 1.57 | 1.83 |
| 428901 | A1929568 | Hs.146668 | KIAA1253 protein | 1.57 | 2.23 |
| 426028 | NM_001110 | Hs.172028 | a disintegrin and metalloproteinase doma | 1.57 | 3.07 |
| 444604 | AW32 7695 | Hs.11441 | chromosome 1 open reading frame 8 | 1.57 | 1.86 |
| 439686 | W40445 | Hs.235857 | ESTs, Weakly similar to I38022 hypotheti | 1.57 | 3.07 |
| 426996 | AW968934 | Hs.173108 | Homo sapiens cDNA: FLJ21897 fis, clone H | 1.57 | 2.01 |
| 447343 | AA256641 | Hs.236894 | ESTs, Highly similar to S02392 alpha-2-m | 1.57 | 2.83 |
| 418942 | AI566004 | Hs.141269 | Homo sapiens cDNA: FLJ21550 fis, clone C | 1.57 | 1.21 |
| 418555 | AI417215 | Hs.87159 | hypothetical protein FLJ12577 | 1.56 | 3.08 |
| 402368 | | | NM_021155*: Homo sapiens CD209 antigen (C | 1.56 | 2.05 |
| 419749 | X73608 | Hs.93029 | sparc/osteonectin, cwcv and kazal-like d | 1.56 | 2.08 |
| 404977 | | | Insulin-like growth factor 2 (somatomedi | 1.56 | 5.50 |
| 441872 | BE567100 | Hs.154938 | hypothetical protein MDS025 | 1.56 | 2.30 |
| 415503 | U36601 | Hs.78473 | N-deacetylase/N-sulfotransferase (hepara | 1.56 | 2.56 |
| 451743 | AW074266 | Hs.23071 | ESTs | 1.56 | 1.85 |
| 423184 | NM_004428 | Hs.1624 | ephrin-A1 | 1.56 | 1.41 |
| 408041 | AW138782 | Hs.243607 | ESTs | 1.56 | 2.21 |
| 416777 | AF146760 | Hs.79844 | DKFZP564M1416 protein | 1.56 | 2.00 |
| 428013 | AF151020 | Hs.181444 | hypothetical protein | 1.56 | 1.53 |
| 410072 | BE384447 | Hs.16034 | hypothetical protein MGC13186 | 1.55 | 1.52 |
| 411495 | AP000693 | Hs.70359 | KIAA0136 protein | 1.55 | 2.88 |
| 408162 | AA993833 | Hs.118527 | ESTs | 1.55 | 2.70 |
| 413350 | U02556 | Hs.75307 | t-complex-associated-testis-expressed 1- | 1.55 | 1.99 |
| 422010 | AA302049 | Hs.31181 | Homo sapiens cDNA: FLJ23230 fis, clone C | 1.55 | 1.60 |
| 425229 | AU076961 | Hs.155212 | methylmalonyl Coenzyme A mutase | 1.55 | 2.57 |
| 425184 | BE278288 | Hs.155048 | Lutheran blood group (Auberger b antigen | 1.55 | 1.45 |
| 419011 | H56244 | Hs.89552 | glutathione S-transferase A2 | 1.55 | 2.77 |
| 417538 | AW050865 | Hs.275711 | hypothetical protein MGC2452 | 1.55 | 2.76 |
| 409806 | AW500960 | | gb: UI-HF-BP0p-aiy-b-01-0-UI.r1 NIH_MGC_5 | 1.55 | 2.45 |
| 402737 | | | Target Exon | 1.54 | 2.58 |
| 419825 | AI754011 | Hs.7326 | ESTs | 1.54 | 1.00 |
| 410001 | AB041036 | Hs.57771 | kallikrein 11 | 1.54 | 0.62 |
| 407813 | AL120247 | Hs.40109 | KIAA0872 protein | 1.54 | 2.33 |
| 415906 | AI751357 | Hs.288741 | Homo sapiens cDNA: FLJ22256 fis, clone H | 1.54 | 2.77 |
| 427886 | AA417083 | Hs.104789 | ESTs | 1.54 | 2.60 |
| 437018 | AA889078 | Hs.187033 | ESTs | 1.54 | 2.48 |
| 415049 | N67334 | Hs.50158 | ESTs | 1.54 | 2.57 |
| 422315 | U16296 | Hs.115176 | T-cell lymphoma invasion and metastasis | 1.54 | 2.57 |
| 413715 | AW851121 | Hs.75497 | Homo sapiens cDNA: FLJ22139 fis,clone H | 1.54 | 1.98 |
| 447144 | AI630759 | Hs.17481 | Homo sapiens clone 24606 mRNA sequence | 1.54 | 2.48 |
| 438924 | BE535511 | | transmembrane trafficking protein | 1.53 | 3.08 |
| 445166 | AI656116 | Hs.147451 | ESTs | 1.53 | 2.08 |
| 414073 | AF068293 | Hs.75737 | pericentriolar material 1 | 1.53 | 1.70 |
| 402378 | | | Target Exon | 1.53 | 2.83 |
| 452316 | AA298484 | Hs.61265 | ESTs, Moderately similar to G786_HUMAN P | 1.53 | 1.60 |
| 450374 | AA397540 | Hs.60293 | Homo sapiens clone 122482 unknown mRNA | 1.53 | 3.59 |
| 402617 | | | C1003551: gi\|6678593\|ref\|NP_033547.1\| win | 1.53 | 2.75 |
| 406837 | R70292 | Hs.156110 | immunoglobulin kappa constant | 1.53 | 1.01 |
| 410573 | AF151057 | Hs.64595 | aminoadipate-semialdehyde dehydrogenase- | 1.53 | 1.23 |
| 426359 | AA376409 | Hs.1 0862 | Homo sapiens cDNA: FLJ23313 fis, clone H | 1.53 | 0.67 |
| 434445 | AI349306 | Hs.11782 | ESTs | 1.53 | 2.80 |
| 452717 | AW160399 | Hs.30376 | hypothetical protein | 1.53 | 2.01 |
| 420465 | AL080276 | Hs.70488 | similar to prokaryotic-type class I pept | 1.53 | 2.25 |
| 437404 | AA868974 | Hs.180992 | ESTs | 1.53 | 2.00 |
| 459192 | AW176180 | | gb: RC2-BT0214-010999-001-E07 BT0214 Homo | 1.52 | 3.20 |
| 446457 | AI300580 | Hs.345281 | ESTs, Moderately similar to ALU1_HUMAN A | 1.52 | 2.35 |
| 441466 | AW673081 | Hs.54828 | ESTs | 1.52 | 1.99 |
| 421810 | AK001718 | Hs.108530 | hypothetical protein FLJ10856 | 1.52 | 2.98 |
| 447769 | AW873704 | Hs.320831 | Homo sapiens cDNA FLJ14597 fis, clone NT | 1.52 | 2.47 |
| 414882 | D79994 | Hs.77546 | Homo sapiens cDNA: FLJ21983 fis, clone H | 1.52 | 2.55 |
| 442169 | W21813 | Hs.8125 | Homo sapiens mRNA; cDNA DKFZp586E1521 (f | 1.52 | 1.31 |
| 404349 | | | Target Exon | 1.52 | 2.74 |
| 416278 | AA356366 | Hs.79137 | protein-L-isoaspartate (D-aspartate) O-m | 1.52 | 2.93 |
| 431846 | BE019924 | Hs.271580 | uroplakin 1B | 1.52 | 1.01 |
| 431958 | X63629 | Hs.2877 | cadherin 3, type 1, P-cadherin (placenta | 1.52 | 0.93 |
| 442670 | BE410050 | Hs.11859 | hypothetical protein FLJ13188 | 1.52 | 2.70 |
| 441617 | AA581863 | Hs.178485 | Homo sapiens cDNA FLJ13919 fis, clone Y7 | 1.52 | 1.65 |
| 440079 | AI557284 | Hs.6900 | ring finger protein 13 | 1.52 | 1.76 |
| 432831 | AI821702 | Hs.115959 | ESTs, Weakly similar to I38022 hypotheti | 1.52 | 2.13 |
| 414320 | U13616 | Hs.75893 | ankyrin 3, node of Ranvier (ankyrin G) | 1.52 | 2.13 |
| 442149 | AB014550 | Hs.8118 | KIAA0650 protein | 1.52 | 1.00 |
| 457747 | AW975000 | | gb: EST387105 MAGE resequences, MAGN Homo | 1.51 | 2.38 |
| 419433 | AA814807 | Hs.7395 | hypothetical protein FLJ23182 | 1.51 | 2.50 |
| 431812 | AA515902 | Hs.130650 | ESTs | 1.51 | 1.64 |
| 415477 | NM_002228 | Hs.78465 | v-jun avian sarcoma virus 17 oncogene ho | 1.51 | 2.62 |
| 447580 | AI953360 | Hs.133487 | ESTs | 1.51 | 2.02 |
| 416926 | H03109 | Hs.108920 | HT018 protein | 1.51 | 2.22 |
| 442755 | W57656 | Hs.109701 | ubiquitin-like 5 | 1.51 | 1.34 |
| 448694 | AA478756 | Hs.194477 | E3 ubiquitin ligase SMURF2 | 1.51 | 2.24 |
| 422675 | BE018517 | Hs.119140 | eukaryotic translation initiation factor | 1.51 | 1.49 |
| 404397 | | | ENSP00000251675*: KIAA1550 protein (Fragm | 1.51 | 2.18 |
| 412927 | AA284018 | Hs.75063 | human immunodeficiency virus type I enha | 1.51 | 1.33 |
| 402371 | | | Target Exon | 1.51 | 3.22 |
| 431730 | AF208856 | Hs.268122 | hypothetical protein | 1.51 | 1.57 |
| 417715 | AW969587 | Hs.86366 | ESTs | 1.51 | 1.59 |
| 451117 | AA015752 | Hs.205173 | ESTs | 1.50 | 2.70 |
| 434727 | H43374 | Hs.7890 | Homo sapiens mRNA for KIAA1671 protein, | 1.50 | 3.53 |
| 442297 | NM_006202 | Hs.89901 | phosphodiesterase 4A, cAMP-specific (dun | 1.50 | 2.24 |
| 425883 | AL137708 | Hs.161031 | Homo sapiens mRNA; cDNA DKFZp434K0322 (f | 1.50 | 1.13 |
| 452658 | N88604 | Hs.30212 | thyroid receptor interacting protein 15 | 1.50 | 1.62 |
| 428695 | AI355647 | Hs.189999 | purinergic receptor (family A group 5) | 1.50 | 1.00 |
| 438967 | H30340 | Hs.173705 | Homo sapiens cDNA: FLJ22050 fis, clone H | 1.50 | 1.05 |
| 419847 | AW390601 | Hs.184544 | Homo sapiens, clone IMAGE: 3355383, mRNA, | 1.50 | 2.53 |
| 431369 | BE184455 | Hs.251754 | secretory leukocyte protease inhibitor( | 1.49 | 0.97 |
| 433265 | AB040971 | Hs.35096 | KIAA1538 protein | 1.49 | 1.44 |
| 408136 | AL041135 | Hs.42959 | KIAA1012 protein | 1.49 | 2.24 |
| 455485 | AA102287 | Hs.26756 | hypothetical protein FLJ20896 | 1.49 | 2.40 |
| 418863 | AL135743 | Hs.25566 | ESTs, Weakly similar to 2004399A chromos | 1.49 | 2.84 |
| 405193 | | | C7000789: gi\|1943947\|gb\|AAC48716.1\| (U901 | 1.48 | 2.20 |
| 408948 | AW296713 | Hs.221441 | ESTs | 1.48 | 2.20 |
| 426088 | AF038007 | Hs.166196 | ATPase, Class I, type 8B, member 1 | 1.48 | 2.24 |
| 405932 | | | C15000305: gi\|3806122\|gb\|AAC69198.1\| (AF0 | 1.48 | 1.48 |
| 454034 | NM_000691 | Hs.575 | aldehyde dehydrogenase 3 family, member | 1.48 | 1.16 |
| 422355 | AW403724 | Hs.300697 | coagulation factor VII (serum prothrombi | 1.48 | 1.20 |
| 428044 | AA093322 | Hs.301404 | RNA binding motif protein 3 | 1.48 | 2.38 |
| 416166 | AW501907 | Hs.261734 | Homo sapiens cDNA: FLJ22807 fis,clone K | 1.48 | 1.28 |
| 430453 | BE387060 | Hs.3903 | Cdc42 effector protein 4; binder of Rho | 1.48 | 2.73 |
| 401600 | BE247275 | | U5 snRNP-specific protein, 116 kD | 1.48 | 2.53 |
| 432638 | AI017717 | | chromosome 21 open reading frame 15 | 1.48 | 2.03 |
| 405194 | | | C7000789: gi\|1943947\|gb\|AAC48716.1\| (U901 | 1.48 | 2.00 |
| 416179 | R19015 | Hs.79067 | MAD (mothers against decapentaplegic, Dr | 1.48 | 1.25 |
| 450272 | AI075170 | Hs.20010 | ESTs | 1.48 | 2.35 |
| 413709 | BE158687 | | gb: CM0-HT0395-280100-169-b09 HT0395 Homo | 1.48 | 2.08 |
| 442607 | AA507576 | Hs.288361 | Homo sapiens cDNA: FLJ22696 fis, clone H | 1.48 | 1.00 |
| 410418 | D31382 | Hs.63325 | transmembrane protease, serine 4 | 1.47 | 1.91 |
| 436566 | BE545586 | Hs.278712 | Homo sapiens cDNA FLJ11074 fis, clone PL | 1.47 | 2.26 |
| 404769 | | | NM_007037*: Homo sapiens a disintegrin-li | 1.47 | 1.24 |
| 420132 | BE079847 | Hs.301914 | gb: RC6-BT0627-220300-012-D06 BT0627 Homo | 1.47 | 2.00 |
| 448356 | AL120837 | Hs.20993 | high-glucose-regulated protein 8 | 1.47 | 2.90 |
| 421628 | AL121317 | Hs.106210 | hypothetical protein FLJ10813 | 1.47 | 4.08 |
| 449059 | AK000566 | Hs.98135 | hypothetical protein FLJ20559 | 1.47 | 3.13 |
| 449029 | N28989 | Hs.22891 | solute carrier family 7 (cationic amino | 1.47 | 1.06 |
| 422119 | AI277829 | Hs.111862 | KIAA0590 gene product | 1.47 | 1.51 |
| 438713 | H16902 | | ESTs | 1.47 | 2.39 |
| 418248 | NM_005000 | Hs.83916 | NM_005000*: Homo sapiens NADH dehydrogena | 1.47 | 1.00 |
| 419125 | AA642452 | Hs.130881 | B-cell CLL/lymphoma 11A (zinc finger pro | 1.46 | 2.20 |
| 420548 | AA278246 | Hs.920 | ESTs | 1.46 | 2.13 |
| 424258 | AA433848 | Hs.107882 | hypothetical protein FLJ10659 | 1.46 | 1.98 |
| 414683 | S78296 | Hs.76888 | hypothetical protein MGC12702 | 1.46 | 1.45 |
| 427045 | H86504 | Hs.173328 | protein phosphatase 2, regulatory subuni | 1.46 | 2.31 |
| 446646 | BE552004 | Hs.26192 | ESTs, Weakly similar to ALU1_HUMAN ALU S | 1.46 | 1.30 |
| 427257 | AI026805 | Hs.97726 | ESTs | 1.46 | 2.48 |
| 422971 | AI879223 | Hs.145409 | RAB, member of RAS oncogene family-like | 1.46 | 1.05 |
| 451334 | AI122691 | Hs.13268 | ESTs | 1.46 | 2.12 |
| 403326 | | | C2000428*: gi\|7705383\|ref\|NP_057536.1\| GC | 1.46 | 2.40 |
| 453827 | AF201948 | Hs.35660 | BUP protein | 1.46 | 1.65 |
| 423599 | AI805664 | Hs.31731 | peroxiredoxin 5 | 1.46 | 1.56 |
| 410691 | AW239226 | Hs.65450 | reticulon 4 | 1.46 | 1.49 |
| 430688 | AL022101 | Hs.104991 | hypothetical protein similar to preferen | 1.46 | 2.45 |
| 438083 | AI949940 | Hs.121924 | ESTs | 1.46 | 2.00 |
| 430713 | AA351647 | Hs.2642 | eukaryotic translation elongation factor | 1.45 | 1.60 |
| 437325 | AF142481 | Hs.5548 | f-box and leucine-rich repeat protein 5 | 1.45 | 1.26 |
| 403342 | | | Target Exon | 1.45 | 2.21 |
| 438808 | M73980 | Hs.129053 | Homo sapiens NOTCH 1 (N1) mRNA, complete | 1.45 | 2.40 |
| 446493 | AK001389 | Hs.15144 | hypothetical protein DKFZp564O043 | 1.45 | 3.65 |
| 414895 | AW894856 | Hs.116278 | Homo sapiens cDNA FLJ13571 fis, clone PL | 1.44 | 2.71 |
| 442072 | AI740832 | Hs.12311 | Homo sapiens clone 23570 mRNA sequence | 1.44 | 1.08 |
| 425723 | NM_014420 | Hs.159311 | dickkopf (Xenopus laevis) homolog 4 | 1.44 | 2.24 |
| 432901 | AI554929 | Hs.281866 | ATPase, H transporting, lysosomal (vacuo | 1.44 | 1.63 |
| 412210 | AW901492 | | gb: RC0-NN1012-270300-031-h10 NN1012 Homo | 1.44 | 2.15 |
| 421685 | AF189723 | Hs.106778 | ATPase, Ca transporting, type 2C, member | 1.44 | 1.83 |
| 428115 | AB023194 | Hs.300855 | KIAA0977 protein | 1.44 | 1.31 |
| 442358 | BE567985 | Hs.18585 | ESTs, Moderately similar to ALU4_HUMAN A | 1.44 | 2.47 |
| 414685 | L39874 | Hs.76894 | dCMP deaminase | 1.44 | 1.25 |
| 413798 | AA336708 | Hs.75546 | capping protein (actin filament) muscle | 1.44 | 1.26 |
| 410937 | AA218564 | Hs.67052 | vacuolar protein sorting 26 (yeast homol | 1.44 | 1.41 |
| 400397 | AJ270770 | | transcription factor 7-like 2 (T-cell sp | 1.44 | 3.43 |
| 405902 | | | Target Exon | 1.44 | 2.65 |
| 433976 | AA620987 | Hs.190268 | ESTs | 1.44 | 2.46 |
| 405376 | | | Target Exon | 1.44 | 2.28 |
| 436086 | Z43133 | Hs.9961 | Home sapiens cDNA: FLJ21954 fis, clone H | 1.44 | 1.34 |
| 418182 | AW016405 | Hs.16648 | ESTs | 1.44 | 2.35 |
| 430307 | BE513442 | Hs.238944 | hypothetical protein FLJ10631 | 1.43 | 1.55 |
| 434924 | AA443164 | Hs.23259 | hypothetical protein FLJ13433 | 1.43 | 2.05 |
| 417821 | BE245149 | Hs.82643 | protein tyrosine kinase 9 | 1.43 | 2.15 |
| 404744 | | | Target Exon | 1.43 | 1.99 |
| 405418 | | | Target Exon | 1.43 | 2.83 |
| 402869 | | | Target Exon | 1.43 | 2.40 |
| 451608 | AA384525 | Hs.26745 | hypothetical protein | 1.43 | 1.22 |
| 424099 | AF071202 | Hs.139336 | ATP-binding cassette, sub-family C (CFTR | 1.43 | 2.45 |
| 401041 | | | C11000425: gi\|4507721\|ref\|NP_003310.1\| ti | 1.43 | 2.90 |
| 417839 | AI815732 | Hs.82712 | fragile X mental retardation, autosomal | 1.43 | 2.84 |
| 409245 | AA361037 | Hs.288036 | tRNA isopentenylpyrophosphate transferas | 1.43 | 2.65 |
| 447808 | NM_007265 | Hs.19673 | suppressor of S. cerevisiae gcr2 | 1.43 | 2.00 |
| 456492 | AA330047 | Hs.191187 | ESTs | 1.43 | 2.73 |
| 449244 | AW859979 | Hs.32204 | ESTs | 1.42 | 1.57 |
| 413094 | H24184 | Hs.25413 | TOLLIP protein | 1.42 | 1.33 |
| 452407 | AA682909 | Hs.29353 | brain-specific protein p25 alpha | 1.42 | 2.50 |
| 407674 | AW064061 | Hs.279145 | ESTs | 1.42 | 2.35 |
| 441297 | AW403084 | Hs.7766 | ubiquitin-conjugating enzyme E2E 1 (homo | 1.42 | 2.20 |
| 421932 | W51778 | Hs.323949 | kangai 1 (suppression of tumorigenicity | 1.42 | 1.48 |
| 426348 | BE466586 | Hs.17433 | hypothetical protein FLJ20967 | 1.42 | 1.83 |
| 432554 | AI479813 | Hs.278411 | NCK-associated protein 1 | 1.42 | 2.46 |
| 431735 | AW977724 | Hs.75968 | thymosin, beta 4, X chromosome | 1.42 | 1.30 |
| 429953 | NM_004376 | Hs.226581 | COX15 (yeast) homolog, cytochrome c oxid | 1.42 | 1.50 |
| 444037 | AV647686 | Hs.42733 | CHMP1.5 protein | 1.42 | 1.38 |
| 402144 | | | Target Exon | 1.42 | 2.38 |
| 456758 | AA325170 | Hs.224627 | ESTs, Weakly similar to FAHUAA alpha-act | 1.42 | 2.23 |
| 452322 | BE566343 | Hs.28988 | glutaredoxin (thioltransferase) | 1.42 | 2.18 |
| 426863 | AL137657 | Hs.172803 | hypothetical protein MGC10327 | 1.41 | 1.38 |
| 410684 | AA088500 | Hs.170298 | ESTs | 1.41 | 1.28 |
| 401784 | | | NM_002280*: Homo sapiens keratin, hair, a | 1.41 | 1.37 |
| 427523 | BE242779 | Hs.179526 | upregulated by 1,25-dihydroxyvitamin D-3 | 1.41 | 1.32 |
| 449269 | AI564682 | Hs.175870 | ESTs | 1.41 | 1.37 |
| 406467 | | | Target Exon | 1.41 | 1.80 |
| 444339 | T96555 | Hs.31562 | ESTs | 1.41 | 2.94 |
| 431563 | AI027643 | Hs.120912 | ESTs | 1.41 | 1.41 |
| 413343 | BE392026 | Hs.334346 | hypothetical protein MGC13045 | 1.41 | 1.21 |
| 447537 | AW295072 | Hs.346408 | ESTs, Weakly similar to AF193556 1 sacsi | 1.41 | 2.07 |
| 428211 | AA424211 | Hs.183176 | ESTs | 1.41 | 1.25 |
| 406248 | | | Target Exon | 1.41 | 2.40 |
| 437412 | BE069288 | Hs.34744 | Homo sapiens mRNA; cDNA DKFZp547C136 (fr | 1.41 | 1.39 |
| 414653 | M24486 | Hs.76768 | procollagen-proline, 2-oxoglutarate 4-di | 1.41 | 1.33 |
| 403885 | | | Target Exon | 1.41 | 2.58 |
| 439459 | AF086279 | Hs.58013 | ESTs | 1.41 | 2.08 |
| 419075 | T84266 | Hs.123927 | ESTs | 1.41 | 2.84 |
| 405022 | | | Target Exon | 1.40 | 2.55 |
| 401346 | BE041451 | | hypothetical protein | 1.40 | 2.38 |
| 415660 | AI909007 | Hs.78563 | ubiquitin-conjugating enzyme E2G 1 (homo | 1.40 | 2.38 |
| 448023 | AI693299 | Hs.170388 | ESTs | 1.40 | 2.38 |
| 435962 | AA702820 | Hs.291294 | ESTs | 1.40 | 2.10 |
| 432480 | AA205475 | Hs.275865 | ribosomal protein S18 | 1.40 | 1.37 |
| 414309 | AK000639 | Hs.758840 | DFZP586A011 protein | 1.40 | 1.18 |
| 440256 | U23841 | Hs.18851 | hypothetical protein FLJ10875 | 1.40 | 1.91 |
| 413809 | L25851 | Hs.851 | integrin, alpha E (antigen CD103, human | 1.40 | 2.80 |
| 408176 | AK001553 | Hs.43436 | adenylate kinase 3 alpha like | 1.40 | 2.73 |
| 433960 | AW629188 | Hs.188929 | ESTs | 1.40 | 1.99 |
| 404178 | | | C6001430*: gi\|4503521\|ref\|NP_001559.1\| mu | 1.40 | 2.83 |
| 402449 | | | Target Exon | 1.40 | 1.51 |
| 455604 | BE011183 | | gb: PM3-2N0218-100500-003-d09 BN0218 Homo | 1.40 | 2.30 |
| 429221 | AI821060 | Hs.198271 | Target CAT | 1.40 | 1.22 |
| 422122 | AA383642 | Hs.111894 | lysosomal-associated protein transmembra | 1.40 | 1.42 |
| 406231 | | | Target Exon | 1.40 | 2.60 |
| 405879 | | | Target Exon | 1.40 | 2.73 |
| 450936 | AI033745 | | gb: ow23a10.x1 Soares_parathyroid_tumor_N | 1.40 | 1.13 |
| 403381 | | | ENSP00000231844*: Ecotropic virus integra | 1.39 | 6.03 |
| 453258 | AW293134 | Hs.32597 | ring finger protein (C3H2C3 type) 6 | 1.39 | 3.20 |
| 448261 | BE244072 | Hs.20815 | macrophage erythroblast attacher | 1.39 | 1.33 |
| 427666 | AI791495 | Hs.180142 | calmodulin-like skin protein (CLSP) | 1.39 | 2.30 |
| 413859 | AW992356 | Hs.8364 | Homo sapiens pyruvate dehydrogenase kina | 1.39 | 1.53 |
| 407704 | BE315072 | Hs.78768 | malignant cell expression-enhanced gene/ | 1.39 | 1.34 |
| 430138 | AA936296 | Hs.234265 | DKFZP586G011 protein | 1.39 | 2.38 |
| 432841 | M93425 | Hs.62 | protein tyrosine phosphatase, non-recept | 1.39 | 2.88 |
| 444051 | N48373 | Hs.10247 | activated leucocyte cell adhesion molecu | 1.39 | 1.34 |
| 440704 | M69241 | Hs.162 | insulin-like growth factor binding prote | 1.39 | 1.61 |
| 450092 | AW139606 | Hs.221057 | ESTs, Weakly similar to ALU1_HUMAN ALU S | 1.39 | 2.78 |
| 400275 | | | NM_006513*: Homo sapiens seryl-tRNA synth | 1.39 | 2.03 |
| 403725 | | | Target Exon | 1.39 | 2.03 |
| 443211 | AI128388 | Hs.143655 | ESTs | 1.39 | 1.83 |
| 421510 | AK000919 | Hs.105191 | hypothetical protein FLJ10057 | 1.39 | 2.83 |
| 430071 | AA355986 | Hs.232068 | transcription factor 8 (represses interl | 1.38 | 3.54 |
| 451545 | AI802128 | Hs.208647 | ESTs | 1.38 | 2.21 |
| 439897 | NM_015310 | Hs.6763 | KIAA0942 protein | 1.38 | 3.65 |
| 423872 | AB020316 | Hs.134015 | uronyl 2-sulfotransferase | 1.38 | 1.00 |
| 410344 | AW978436 | Hs.62515 | KIAA0494 gene product | 1.38 | 2.25 |
| 404439 | | | ENSP00000067222*: Mitochondrial 28S ribos | 1.38 | 2.25 |
| 448581 | NM_002709 | Hs.21537 | protein phosphatase 1, catalytic subunit | 1.38 | 1.47 |
| 408569 | BE066047 | Hs.86412 | chromosome 9 open reading frame 5 | 1.38 | 1.27 |
| 447643 | H10767 | Hs.238465 | nGAP-like protein | 1.38 | 1.22 |
| 401593 | | | Target Exon | 1.38 | 2.58 |
| 403807 | | | NM_031889: Homo sapiens enamelin (ENAM), | 1.38 | 2.38 |
| 406356 | N47812 | | CGI-35 protein | 1.38 | 2.25 |
| 401886 | | | NM_021783: Homo sapiens XEDAR (XEDAR), mR | 1.38 | 2.00 |
| 421110 | AJ250717 | Hs.1355 | cathepsin E | 1.38 | 8.93 |
| 427449 | AW946384 | Hs.178112 | DNA segment single copy probe LNS-CAI/L | 1.38 | 1.44 |
| 427451 | AI690916 | Hs.178137 | transducer of ERBB2, 1 | 1.38 | 2.81 |
| 440681 | AW449696 | Hs.166547 | ESTs | 1.38 | 2.95 |
| 419590 | AF005043 | Hs.91390 | poly (ADP-ribose) glycohydrolase | 1.38 | 2.10 |
| 446044 | H67567 | Hs.13572 | calcium modulating ligand | 1.37 | 2.62 |
| 400967 | | | Target Exon | 1.37 | 3.12 |
| 414506 | AF075337 | Hs.76293 | thymosin, beta 10 | 1.37 | 1.18 |
| 402599 | | | NM_021186*: Homo sapiens zona pellucida g | 1.37 | 2.68 |
| 422932 | AI191813 | Hs.308220 | ESTs | 1.37 | 2.38 |
| 433889 | AK002082 | Hs.3623 | hypothetical protein FLJ11220 | 1.37 | 2.23 |
| 429802 | H09548 | Hs.5367 | ESTs, Weakly similar to I38022 hypotheti | 1.37 | 2.25 |
| 443856 | AK000574 | Hs.9908 | nitrogen fixation cluster-like | 1.37 | 1.28 |
| 453489 | AA300067 | Hs.33032 | hypothetical protein DKFZp434N185 | 1.37 | 2.15 |
| 424670 | W61215 | Hs.116651 | epithelial V-like antigen 1 | 1.37 | 1.66 |
| 428995 | AW004975 | Hs.194716 | MAD (mothers against decapentaplegic, Dr | 1.37 | 1.33 |
| 441551 | AA318224 | Hs.296141 | ESTs | 1.37 | 2.95 |
| 450528 | NM_014072 | Hs.25063 | PRO0461 protein | 1.37 | 1.19 |
| 427605 | NM_000997 | Hs.337445 | ribosomal protein L37 | 1.37 | 1.31 |
| 459237 | AA031675 | Hs.31917 | Homo sapiens, clone MGC: 9658, mRNA, comp | 1.37 | 2.50 |
| 413691 | AB023173 | Hs.75478 | ATPase, Class VI, type 11B | 1.37 | 1.31 |
| 404906 | | | NM_025213: Homo sapiens spectrin, beta, n | 1.36 | 3.08 |
| 436246 | AW450963 | Hs.119991 | ESTs | 1.36 | 1.00 |
| 441478 | AA350018 | Hs.301342 | hypothetical protein MGC4342 | 1.36 | 1.43 |
| 419715 | AF070523 | Hs.92384 | vitamin A responsive; cytoskeleton relat | 1.36 | 1.28 |
| 426251 | M24283 | Hs.168383 | intercellular adhesion molecule 1 (CD54) | 1.36 | 2.16 |
| 400129 | | | Eos Control | 1.36 | 2.03 |
| 450447 | AF212223 | Hs.25010 | hypothetical protein P15-2 | 1.36 | 2.13 |
| 434697 | AL133033 | Hs.4084 | KIAA1025 protein | 1.36 | 2.01 |
| 430308 | BE540865 | Hs.238990 | cyclin-dependent kinase inhibitor 1B (p2 | 1.36 | 2.03 |
| 434767 | AF153201 | | C2H2 (Kruppel-type) zinc finger protein | 1.36 | 2.87 |
| 459729 | AL037285 | Hs.289848 | EST, Weakly similar to ALU4_HUMAN ALU SU | 1.36 | 1.27 |
| 426653 | AA530892 | Hs.171695 | deal specificity phosphatase 1 | 1.35 | 2.20 |
| 408912 | AB011084 | Hs.48924 | KIAA0512 gene product; ALEX2 | 1.35 | 2.68 |
| 409844 | AW502336 | | gb: UI-HF-BR0p-aka-b-05-0-UI.r1 NIH_MGC_5 | 1.35 | 2.29 |
| 402517 | | | Target Exon | 1.35 | 2.10 |
| 447042 | AB035863 | Hs.182217 | succinate-CoA ligase, ADP-forming, beta | 1.35 | 1.25 |
| 405000 | | | Target Exon | 1.35 | 2.32 |
| 452065 | AK000360 | Hs.27721 | Wolf-Hirschhorn syndrome candidate 1-lik | 1.35 | 2.36 |
| 404666 | | | C9000748: gi\|8324209\|gb\|AAB34384.2\| (S775 | 1.35 | 2.55 |
| 451081 | AI078645 | Hs.431 | murine leukemia viral (bmi-1) oncogene h | 1.35 | 1.70 |
| 427979 | BE379776 | Hs.181309 | proteasome (prosome, macropain) subunit, | 1.35 | 2.23 |
| 435825 | R16702 | Hs.91147 | ESTs | 1.35 | 2.39 |
| 426469 | BE297886 | Hs.293970 | methylmalonate-semialdehyde dehydrogenas | 1.35 | 1.40 |
| 447002 | BE242866 | Hs.16933 | HepA-related protein | 1.34 | 2.88 |
| 410946 | AW811502 | | gb: QV2-ST0145-061299-015-b04 ST0145 Homo | 1.34 | 2.02 |
| 454383 | AW500332 | Hs.11114 | hypothetical protein dJ1181N3.1 | 1.34 | 2.13 |
| 440512 | AA887845 | Hs.19673 | suppressor of S. cerevisiae gcr2 | 1.34 | 2.05 |
| 409865 | AW502208 | | gb: UI-HF-BR0p-aju-e-09-0-UI.r1 NIH_MGC_5 | 1.34 | 2.63 |
| 447390 | X95384 | Hs.18426 | translational inhibitor protein p14.5 | 1.34 | 1.00 |
| 450293 | N36754 | Hs.171118 | hypothetical protein FLJ00026 | 1.34 | 2.45 |
| 445831 | NM_006055 | Hs.13351 | LanC (bacterial lantibiotic synthetase c | 1.34 | 1.60 |
| 418610 | AW245993 | Hs.223394 | hypothetical protein MGC2742 | 1.34 | 1.39 |
| 441946 | AW298716 | Hs.120775 | ESTs | 1.34 | 2.30 |
| 446192 | H49944 | Hs.14231 | selenoprotein W, 1 | 1.34 | 1.17 |
| 416285 | BE537973 | Hs.48617 | Homo sapiens cDNA FLJ12540 fis, clone NT | 1.34 | 2.22 |
| 425590 | AI954686 | Hs.158321 | beaded filament structural protein 2, ph | 1.34 | 2.50 |
| 407498 | U28131 | | gb: Human HMGI-C chimeric transcript mRNA | 1.34 | 2.13 |
| 441331 | AI216764 | Hs.149971 | ESTs, Moderately similar to ALUB_HUMAN! | 1.34 | 2.05 |
| 411789 | AF245505 | Hs.72157 | Adlican | 1.34 | 1.27 |
| 420542 | NM_000505 | Hs.1321 | coagulation factor XII (Hageman factor) | 1.33 | 1.25 |
| 413892 | AI878921 | Hs.75607 | myristoylated alanine-rich protein kinas | 1.33 | 1.41 |
| 439750 | AL359053 | Hs.57664 | Homo sapiens mRNA full length insert cDN | 1.33 | 1.99 |
| 414861 | AL119396 | Hs.77508 | glutamate dehydrogenase 1 | 1.33 | 1.66 |
| 421687 | AL035306 | Hs.106823 | hypothetical protein MGC14797 | 1.33 | 2.18 |
| 410846 | AW807057 | | gb: MR4-ST0062-031199-018-b03 ST0062 Homo | 1.33 | 2.07 |
| 443937 | R66571 | Hs.24601 | ESTs | 1.33 | 2.02 |
| 432360 | BE045243 | Hs.274416 | Target CAT | 1.33 | 1.12 |
| 443119 | AA312264 | Hs.7980 | hypothetical protein MGC12966 | 1.33 | 2.68 |
| 438464 | AA669735 | Hs.324743 | protein phosphatase 4 regulatory subunit | 1.33 | 1.99 |
| 401371 | | | ENSP00000198192*: BA438F9.1 (novel protei | 1.33 | 1.10 |
| 405443 | | | Target Exon | 1.33 | 2.11 |
| 453764 | BE008180 | Hs.282846 | Homo sapiens cDNA FLJ14353 fis, clone Y7 | 1.33 | 2.88 |
| 424924 | AL039103 | Hs.153834 | pumilio (Drosophila) homolog 1 | 1.33 | 1.24 |
| 453555 | N23574 | Hs.123649 | ESTs, Moderately similar to ALU7_HUMAN A | 1.33 | 2.23 |
| 404343 | | | C7002191*: gi\|5053028\|gb\|AAD38811.1\|AF155 | 1.33 | 1.04 |
| 412383 | AW947577 | | gb: RC0-MT0004-140300-031-b09 MT0004 Homo | 1.33 | 2.06 |
| 404250 | | | Target Exon | 1.33 | 2.53 |
| 413899 | AF083892 | Hs.75608 | tight junction protein 2 (zona occludens | 1.33 | 2.81 |
| 422716 | AI702835 | Hs.124475 | ESTs, Weakly similar to YEF4_YEAST HYPOT | 1.33 | 2.30 |
| 448862 | AI351979 | Hs.152717 | hypothetical protein FLJ13725 | 1.33 | 1.08 |
| 409540 | AW409569 | | gb: fh01e09.x1 NIH_MGC_17 Homo sapiens cD | 1.33 | 2.18 |
| 431186 | NM_012249 | Hs.250697 | ras-like protein | 1.32 | 1.39 |
| 402754 | | | NM_022469*: Homo sapiens hypothetical pro | 1.32 | 1.16 |
| 420798 | W93774 | Hs.99936 | keratin 10 (epidermolytic hyperkeratosis | 1.32 | 2.02 |
| 459710 | AI701596 | Hs.121592 | ESTs | 1.32 | 2.70 |
| 435192 | AK000739 | Hs.4835 | eukaryotic translation initiation factor | 1.32 | 2.22 |
| 401383 | | | Target Exon | 1.32 | 2.18 |
| 453394 | AW960474 | Hs.40289 | ESTs | 1.32 | 2.20 |
| 421820 | AW662990 | Hs.294133 | heme-binding protein | 1.32 | 1.24 |
| 444047 | AI097452 | Hs.135095 | ESTs | 1.32 | 2.95 |
| 440860 | R10482 | Hs.132876 | ESTs | 1.32 | 2.83 |
| 425808 | AA364109 | Hs.177990 | ESTs | 1.32 | 2.11 |
| 456558 | BE410992 | Hs.258730 | heme-regulated initiation factor 2-alpha | 1.32 | 2.05 |
| 447015 | AB033029 | Hs.16953 | KIAA1203 protein | 1.32 | 1.30 |
| 414015 | AA340987 | Hs.75693 | prolylcarboxypeptidase (angiotensinase C | 1.32 | 1.39 |
| 414843 | BE386038 | Hs.77492 | heterogeneous nuclear ribonucleoprotein | 1.32 | 1.26 |
| 424058 | AL121516 | Hs.138617 | thyroid hormone receptor interactor 12 | 1.32 | 2.01 |
| 401196 | | | Target Exon | 1.32 | 2.13 |
| 450147 | AW373713 | Hs.146324 | CGI-145 protein | 1.32 | 1.32 |
| 422699 | BE410590 | Hs.119257 | ems1 sequence (mammary tumor and squamou | 1.32 | 1.33 |
| 405172 | | | Target Exon | 1.32 | 2.11 |
| 434087 | AF116675 | Hs.334476 | hypothetical protein PRO1942 | 1.32 | 2.30 |
| 416720 | H05435 | Hs.11110 | hypothetical protein MGC2508 | 1.32 | 2.18 |
| 426621 | NM_001329 | Hs.171391 | C-terminal binding protein 2 | 1.32 | 1.53 |
| 442685 | AB033017 | Hs.8594 | KIAA1191 protein | 1.32 | 1.43 |
| 443879 | Z28462 | Hs.9927 | Homo sapiens mRNA; cDNA DKFZp564D156 (fr | 1.31 | 2.24 |
| 405180 | NM_002649 | | phosphoinositide-3-kinase, catalytic, ga | 1.31 | 1.36 |
| 417365 | D50683 | Hs.82028 | transforming growth factor, beta recepto | 1.31 | 0.98 |
| 402087 | | | Target Exon | 1.31 | 1.31 |
| 429323 | NM_001649 | Hs.2391 | apical protein, Xenopus laevis-like | 1.31 | 2.05 |
| 409935 | AW511413 | Hs.278025 | ESTs | 1.31 | 1.20 |
| 430235 | BE268048 | Hs.236494 | RAB10, member RAS oncogene family | 1.31 | 1.31 |
| 400172 | | | Eos Control | 1.31 | 1.05 |
| 421742 | AW970004 | Hs.107528 | androgen induced protein | 1.31 | 1.79 |
| 404273 | | | Target Exon | 1.31 | 2.35 |
| 416204 | AW972270 | Hs.144054 | ESTs | 1.31 | 2.15 |
| 435076 | AW298113 | Hs.92909 | SON DNA binding protein | 1.31 | 2.05 |
| 452497 | AA732153 | Hs.27865 | Homo sapiens cDNA: FLJ21333 fis, clone C | 1.30 | 1.57 |
| 404596 | | | Target Exon | 1.30 | 2.23 |
| 419080 | AW150835 | Hs.18878 | hypothetical protein FLJ21620 | 1.30 | 1.15 |
| 427195 | W27230 | Hs.173912 | eukaryotic translation initiation factor | 1.30 | 1.34 |
| 438129 | AA778647 | | gb: af87d03.s1 Soares_testis_NHT Homo sap | 1.30 | 2.55 |
| 402138 | | | Target Exon | 1.30 | 2.09 |
| 404029 | | | NM_018936*: Homo sapiens protocadherin be | 1.30 | 2.83 |
| 402731 | AL042818 | | E3 ubiquitin ligase SMURF1 | 1.30 | 3.32 |
| 458766 | AW183618 | Hs.55610 | solute carrier family 30 (zinc transport | 1.30 | 1.56 |
| 434585 | AW451715 | Hs.184075 | ESTs, Weakly similar to ALU1_HUMAN ALU S | 1.30 | 2.73 |
| 417219 | AW973473 | Hs.220936 | ESTs | 1.30 | 2.45 |
| 428125 | AA393071 | Hs.182579 | leucine aminopeptidase | 1.30 | 2.00 |
| 416188 | BE157260 | Hs.79070 | v-myc avian myelocytomatosis viral oncog | 1.30 | 1.00 |
| 444681 | AJ243937 | Hs.288316 | chromosome 6 open reading frame 9 | 1.30 | 0.94 |
| 406621 | X57809 | Hs.181125 | immunoglobulin lambda locus | 1.29 | 1.02 |
| 436663 | AW410458 | Hs.5258 | chromosome 11 open reading frame2 | 1.29 | 1.20 |
| 417250 | N58241 | Hs.332115 | ESTs | 1.29 | 3.43 |
| 434978 | AA321238 | Hs.4310 | eukaryotic translation initiation factor | 1.29 | 1.91 |
| 448079 | R76981 | | thyroid hormone receptor-associated prot | 1.29 | 2.01 |
| 450626 | AW190989 | Hs.1508 | insulin-degrading enzyme | 1.29 | 2.09 |
| 456059 | BE543127 | Hs.336948 | Homo sapiens, clone IMAGE: 3530891, mRNA, | 1.29 | 2.23 |
| 417809 | H75797 | Hs.233550 | zinc finger protein 208 | 1.29 | 2.20 |
| 454771 | AW819939 | Hs.273629 | ESTs | 1.29 | 2.10 |
| 413895 | BE178160 | | gb: RC3-HT0600-060400-022-h10 HT0600 Homo | 1.29 | 2.08 |
| 404649 | | | Target Exon | 1.29 | 1.32 |
| 440676 | NM_004987 | Hs.112378 | LIM and senescent cell antigen-like doma | 1.29 | 2.08 |
| 405891 | | | Target Exon | 1.29 | 2.00 |
| 418965 | AI002238 | Hs.11482 | splicing factor, arginine/serine-rich 11 | 1.29 | 2.41 |
| 412824 | AW958075 | Hs.11261 | small proline-rich protein 2A | 1.29 | 1.27 |
| 420037 | BE299598 | Hs.135569 | hypothetical protein FLJ14708 | 1.29 | 1.23 |
| 459221 | BE246522 | Hs.306121 | leukocyte receptor cluster (LRC) encoded | 1.28 | 2.48 |
| 458651 | AW612481 | Hs.104105 | ESTs | 1.28 | 2.35 |
| 422984 | W28614 | | chorionic somatomammotropin hormone 1 (p | 1.28 | 1.37 |
| 459365 | BE067754 | | gb: MR4-BT0358-140400-006-g10 BT0358 Homo | 1.28 | 1.06 |
| 418254 | AA732511 | Hs.86650 | ESTs | 1.28 | 2.38 |
| 402474 | | | NM_004079: Homo sapiens cathepsin S (CTSS | 1.28 | 1.99 |
| 448456 | AI521830 | Hs.171050 | ESTs | 1.28 | 2.18 |
| 450098 | W27249 | Hs.8109 | hypothetical protein FLJ21080 | 1.28 | 1.68 |
| 405053 | | | Target Exon | 1.28 | 3.23 |
| 428915 | AI041278 | Hs.87908 | Snf2-related CBP activator protein | 1.28 | 4.25 |
| 443721 | AW450451 | Hs.266355 | ESTs | 1.28 | 1.15 |
| 452047 | N35953 | Hs.43510 | ESTs, Weakly similar to BOX B BINDING FA | 1.28 | 2.30 |
| 440213 | AW246253 | Hs.7043 | succinate-CoA ligase, GDP-forming, alpha | 1.28 | 1.19 |
| 452900 | AA626794 | | prothymosin, alpha (gene sequence 28) | 1.28 | 1.27 |
| 418721 | NM_002731 | Hs.87773 | protein kinase, cAMP-dependent, catalyti | 1.28 | 2.76 |
| 458911 | AA373131 | Hs.24322 | ATPase, H transporting, lysosomal (vacuo | 1.28 | 1.21 |
| 444250 | R40815 | Hs.12396 | ESTs, Weakly similar to 2004399A chromos | 1.28 | 2.43 |
| 431631 | AA548906 | Hs.122244 | ESTs | 1.27 | 1.51 |
| 447966 | AA340605 | Hs.105887 | ESTs, Weakly similar to Homolog of rat Z | 1.27 | 3.08 |
| 430316 | NM_000875 | Hs.239176 | insulin-like growth factor 1 receptor | 1.27 | 1.37 |
| 416272 | AA178882 | | gb: zp38b09.r1 Stratagene muscle 937209 H | 1.27 | 2.00 |
| 437456 | AL047045 | Hs.60293 | Homo sapiens clone 122482 unknown mRNA | 1.27 | 3.18 |
| 456327 | H68741 | Hs.38774 | ESTs | 1.27 | 2.35 |
| 403349 | NM_001406 | | ephrin-B3 | 1.27 | 2.28 |
| 428821 | H91282 | Hs.286232 | Homo sapiens cDNA: FLJ23190 fis, clone L | 1.27 | 2.13 |
| 454555 | AW807095 | | gb: MR4-ST0062-040100-024-e02 ST0062 Homo | 1.27 | 2.05 |
| 406872 | AI760903 | | gb: wi09h08.x1 NCI_CGAP_CLL1 Homo sapiens | 1.27 | 1.44 |
| 401720 | | | NM_014587*: Homo sapiens SRY (sex determi | 1.27 | 2.07 |
| 400082 | | | Eos Control | 1.27 | 1.26 |
| 420183 | W92885 | Hs.143408 | ESTs | 1.27 | 2.24 |
| 411579 | AC005258 | Hs.70830 | U6 snRNA-associated Sm-like protein LSm7 | 1.27 | 1.17 |
| 402191 | | | NM_021733*: Homo sapiens testis-specific | 1.27 | 2.44 |
| 457118 | AI245525 | Hs.182469 | Homo sapiens mRNA; cDNA DKFZp564K1972 (f | 1.27 | 2.17 |
| 408576 | NM_003542 | Hs.46423 | H4 histone family, member G | 1.27 | 2.78 |
| 452826 | BE245286 | Hs.301636 | peroxisomal biogenesis factor 6 | 1.27 | 3.15 |
| 414909 | R80316 | Hs.132569 | PP2135 protein | 1.27 | 1.37 |
| 416114 | AI695549 | Hs.183868 | glucuronidase, beta | 1.26 | 2.48 |
| 455476 | AW948172 | | gb: RC0-MT0013-280300-021-b06 MT0013 Homo | 1.26 | 2.18 |
| 445926 | AF054284 | Hs.334826 | splicing factor 3b, subunit 1, 155 kD | 1.26 | 1.35 |
| 432647 | AI807481 | Hs.278581 | fibroblast growth factor receptor 2 (bac | 1.26 | 1.16 |
| 405436 | | | Target Exon | 1.26 | 2.38 |
| 406140 | | | Target Exon | 1.26 | 3.20 |
| 426201 | AW182614 | Hs.128499 | ESTs | 1.26 | 1.17 |
| 433334 | AI927208 | Hs.231958 | matrix metalloproteinase 28 | 1.26 | 2.30 |
| 423262 | NM_005479 | Hs.126057 | frequently rearranged in advanced T-cell | 1.26 | 2.61 |
| 422929 | AA356694 | Hs.94011 | ESTs, Weakly similar to MGB4_HUMAN MELAN | 1.26 | 2.11 |
| 445605 | AI906088 | Hs.87159 | hypothetical protein FLJ12577 | 1.26 | 3.11 |
| 425050 | BE391854 | Hs.7970 | gb: 601285394F1 NIH_MGC_44 Homo sapiens c | 1.26 | 2.18 |
| 420539 | AA282735 | Hs.44004 | AD031 protein | 1.26 | 2.03 |
| 437352 | AL353957 | Hs.284181 | hypothetical protein DKFZp434P0531 | 1.25 | 1.19 |
| 456535 | AA305079 | Hs.1342 | cytochrome c oxidase subunit Vb | 1.25 | 1.18 |
| 434202 | BE382411 | Hs.3764 | guanylate kinase 1 | 1.25 | 1.14 |
| 439528 | BE613180 | Hs.288368 | Homo sapiens cDNA: FLJ21314 fis, clone C | 1.25 | 2.12 |
| 400178 | | | Eos Control | 1.25 | 2.15 |
| 430023 | AA158243 | Hs.227729 | FK506-binding protein 2 (13 kD) | 1.25 | 1.20 |
| 412841 | AI751157 | Hs.101395 | hypothetical protein MGC11352 | 1.25 | 1.39 |
| 425655 | BE614551 | Hs.738 | ribosomal protein L14 | 1.25 | 1.22 |
| 449636 | AI656608 | Hs.281328 | ESTs, Weakly similar to T00378 KIAA0641 | 1.25 | 3.00 |
| 418406 | X73501 | Hs.84905 | cytokeratin 20 | 1.24 | 2.11 |
| 414570 | Y00285 | Hs.76473 | insulin-like growth factor 2 receptor | 1.24 | 1.14 |
| 436967 | AA761729 | Hs.136705 | ESTs | 1.24 | 2.53 |
| 457216 | AA452554 | Hs.283697 | ESTs, Weakly similar to A41796 neural re | 1.24 | 2.18 |
| 418414 | J04977 | Hs.84981 | X-ray repair complementing defective rep | 1.24 | 1.35 |
| 423217 | NM_000094 | Hs.1640 | collagen, type VII, alpha 1 (epidermolys | 1.24 | 0.92 |
| 421733 | AL119671 | Hs.1420 | fibroblast growth factor receptor 3 (ach | 1.24 | 2.71 |
| 402329 | | | NM_006505*: Homo sapiens poliovirus recep | 1.24 | 1.13 |
| 447525 | AF151031 | Hs.300631 | hypothetical protein | 1.24 | 1.07 |
| 445939 | BE018658 | Hs.141003 | Homo sapiens cDNA: FLJ21691 fis, clone C | 1.24 | 2.23 |
| 421936 | AB040884 | Hs.109694 | KIAA1451 protein | 1.24 | 2.15 |
| 433681 | AI004377 | Hs.200360 | Homo sapiens cDNA FLJ13027 fis, clone NT | 1.24 | 2.15 |
| 426717 | N90977 | Hs.49690 | Homo sapiens mRNA; cDNA DKFZp434D2328 (f | 1.24 | 2.14 |
| 404751 | T70445 | | ribosomal protein L9 | 1.24 | 1.30 |
| 411456 | AW847588 | | gb: IL3-CT0213-161299-038-G09 CT0213 Homo | 1.24 | 2.35 |
| 425417 | AF098948 | Hs.157113 | coenzyme Q, 7 (rat, yeast) homolog | 1.24 | 2.88 |
| 434508 | AI648601 | Hs.118012 | ESTs | 1.24 | 2.03 |
| 428284 | AA535762 | Hs.183435 | NM_004545: Homo sapiens NADH dehydrogenas | 1.24 | 1.59 |
| 418597 | AK001678 | Hs.86337 | similar to DNA-directed RNA polymerase I | 1.24 | 2.27 |
| 414191 | AW250089 | Hs.75807 | PDZ and LIM domain 1 (elfin) | 1.24 | 1.53 |
| 449210 | AI635363 | Hs.345517 | ESTs | 1.24 | 2.18 |
| 439551 | W72062 | Hs.11112 | ESTs | 1.24 | 2.13 |
| 426244 | AI064808 | Hs.168289 | saccinate dehydrogenase complex, subunit | 1.23 | 1.06 |
| 453635 | BE148082 | Hs.24724 | MFH-amplified sequences with leucine-ric | 1.23 | 1.34 |
| 429617 | X89984 | Hs.211563 | B-cell CLL/lymphoma 7A | 1.23 | 2.27 |
| 434943 | AI929819 | Hs.92909 | chromosome 21 open reading frame 50 | 1.23 | 2.10 |
| 417010 | NM_006225 | Hs.80776 | phospholipase C, delta 1 | 1.23 | 1.21 |
| 426508 | W23184 | Hs.170171 | glutamate-ammonia ligase (glatamine synt | 1.23 | 1.37 |
| 434055 | AF168712 | Hs.3726 | x 003 protein | 1.23 | 1.58 |
| 438363 | AI886351 | Hs.22353 | hypothetical protein FLJ21952 | 1.23 | 2.44 |
| 450937 | R49131 | Hs.26267 | ATP-dependant interferon response protei | 1.23 | 2.28 |
| 407018 | U49869 | | NM_018955: Homo sapiens ubiquitin B (UBB) | 1.23 | 1.44 |
| 444981 | AW855398 | Hs.12210 | hypothetical protein FLJ13732 similar to | 1.23 | 1.19 |
| 440112 | AA099014 | Hs.231029 | Homo sapiens, clone MGC: 15961, mRNA, com | 1.22 | 2.07 |
| 426672 | AW270555 | Hs.171774 | hypothetical protein | 1.22 | 1.16 |
| 404956 | | | C1003210*: gi\|6912582\|ref\|NP_036524.1\| pe | 1.22 | 2.18 |
| 435088 | NM_000481 | Hs.102 | aminomethyltransferase (glycine cleavage | 1.22 | 1.08 |
| 438588 | AW274454 | Hs.6318 | peroxisomal short-chain alcohol dehydrog | 1.22 | 1.02 |
| 434454 | AF217798 | Hs.3850 | LIS1-interacting protein NUDEL; endoolig | 1.22 | 1.27 |
| 425689 | W16480 | Hs.24283 | ESTs, Moderately similar to reduced expr | 1.22 | 2.52 |
| 428755 | D87454 | Hs.192966 | KIAA0265 protein | 1.22 | 1.16 |
| 420685 | AA279362 | | gb: zs84d04.r1 NCI_CGAP_GCB1 Homo sapiens | 1.22 | 2.75 |
| 458991 | AI743502 | | gb: wf63h12.x2 Soares_NFL_T_GBC_S1 Homo s | 1.22 | 2.39 |
| 414825 | X06370 | Hs.77432 | epidermal growth factor receptor (avian | 1.22 | 1.00 |
| 434023 | AI277883 | Hs.146141 | ESTs | 1.22 | 2.12 |
| 430801 | AI580935 | Hs.105698 | ESTs | 1.22 | 2.53 |
| 414880 | AW247305 | Hs.119140 | eukaryotic translation initiation factor | 1.21 | 1.16 |
| 454144 | BE280478 | Hs.182695 | hypothetical protein MGC3243 | 1.21 | 1.04 |
| 404790 | | | C12001707*: gi\|7305215\|ref\|NP_038599.1\| k | 1.21 | 2.05 |
| 403943 | | | C5000355: gi\|4503225\|ref\|NP_000765.1\| cyt | 1.21 | 2.05 |
| 400201 | | | NM_006156*: Homo sapiens neural precursor | 1.21 | 1.35 |
| 421005 | AW293089 | Hs.33263 | ESTs | 1.21 | 2.02 |
| 414774 | X02419 | Hs.77274 | plasminogen activator, urokinase | 1.21 | 1.11 |
| 400789 | | | C11001367*: gi\|1076205\|pir∥S50754 hypoth | 1.21 | 1.06 |
| 412853 | M34175 | Hs.74626 | adaptor-related protein complex 2, beta | 1.21 | 1.24 |
| 449709 | BE410592 | Hs.23918 | hypothetical protein PP5395 | 1.20 | 1.27 |
| 413726 | AJ278465 | Hs.75510 | annexin A11 | 1.20 | 1.14 |
| 428485 | NM_002950 | Hs.2280 | ribophorin I | 1.20 | 1.24 |
| 405163 | | | C5000561*: gi\|7513700\|pir∥T14151 Inv pro | 1.20 | 1.11 |
| 415887 | NM_003375 | Hs.78902 | voltage-dependent anion channel 2 | 1.20 | 1.16 |
| 434468 | N29309 | Hs.39288 | ESTs | 1.20 | 3.20 |
| 446843 | AW135925 | Hs.98798 | hypothetical protein MGC11332 | 1.20 | 2.25 |
| 432642 | BE297635 | Hs.3069 | heat shock 70 kD protein 9B (mortalin-2) | 1.20 | 2.18 |
| 448242 | R60646 | Hs.20768 | HSPC189 protein | 1.20 | 1.22 |
| 415753 | U52819 | Hs.78781 | vascular endothelial growth factor B | 1.20 | 1.05 |
| 442156 | AI690586 | Hs.29403 | hypothetical protein FLJ22060 | 1.20 | 1.18 |
| 408824 | L80005 | Hs.48375 | small nuclear ribonucleoprotein polypept | 1.20 | 1.45 |
| 430012 | NM_015373 | Hs.227637 | chromosome 22 open reading frame 2 | 1.20 | 1.03 |
| 413392 | AW021404 | Hs.13021 | ESTs | 1.20 | 2.07 |
| 401286 | | | Target Exon | 1.20 | 2.08 |
| 415665 | AI097276 | Hs.274430 | surfeit 6 | 1.20 | 2.53 |
| 456562 | AA306049 | Hs.102669 | DKFZP434O125 protein | 1.20 | 2.40 |
| 408988 | AL119844 | Hs.49476 | Homo sapiens clone TUA8 Cri-du-chat regi | 1.20 | 3.45 |
| 427818 | AW511222 | Hs.193765 | ESTs | 1.19 | 2.99 |
| 406404 | | | NM_002162*: Homo sapiens intercellular ad | 1.19 | 1.17 |
| 400124 | | | Eos Control | 1.19 | 2.12 |
| 416023 | AA173029 | | gb: zp05e01.r1 Stratagene ovarian cancer | 1.19 | 2.45 |
| 427751 | AF000152 | Hs.180669 | conserved gene amplified in osteosarcoma | 1.19 | 1.07 |
| 401204 | | | ENSP00000252232*: Sterol regulatory eleme | 1.19 | 2.40 |
| 446771 | AA128965 | Hs.60679 | TATA box binding protein (TBP)-associate | 1.19 | 2.03 |
| 429673 | AA884407 | Hs.211595 | protein tyrosine phosphatase, non-recept | 1.18 | 2.36 |
| 400130 | | | Eos Control | 1.18 | 2.58 |
| 405365 | | | CX001212*: gi\|7861932\|gb\|AAF70445.1\| (AF2 | 1.18 | 2.38 |
| 406181 | | | Target Exon | 1.18 | 2.18 |
| 422559 | AW247696 | Hs.155839 | hypothetical protein MGC12934 | 1.18 | 2.13 |
| 409524 | AW402151 | Hs.54673 | tumor necrosis factor (ligand) superfami | 1.18 | 1.07 |
| 438446 | AW137476 | Hs.135204 | Homo sapiens cDNA FLJ13884 fis, clone TH | 1.18 | 2.11 |
| 447980 | AI703397 | Hs.202355 | ESTs | 1.18 | 2.02 |
| 425503 | W92517 | Hs.158203 | actin binding LIM protein 1 | 1.18 | 1.29 |
| 411469 | T09997 | Hs.70327 | cysteine-rich protein 2 | 1.18 | 0.99 |
| 409162 | H25530 | Hs.50868 | solute carrier family 22 (organic cation | 1.17 | 1.04 |
| 429986 | AF092047 | Hs.227277 | sine oculis homeobox (Drosophila) homolo | 1.17 | 1.00 |
| 420869 | X58964 | Hs.123638 | regulatory factor X, 1 (influences HLA c | 1.17 | 2.58 |
| 425943 | H46986 | Hs.31861 | ESTs | 1.17 | 2.25 |
| 415376 | R35960 | Hs.180711 | Homo sapiens, Similar to hypothetical pr | 1.17 | 2.13 |
| 420588 | AF000982 | Hs.147916 | DEAD/H (Asp-Glu-Ala-Asp/His) box polypep | 1.17 | 2.38 |
| 457205 | AI905780 | Hs.198272 | Target CAT | 1.17 | 1.11 |
| 407970 | AW403814 | Hs.41714 | BCL2-associated athanogene | 1.16 | 3.60 |
| 440214 | AA247118 | Hs.7049 | hypothetical protein FLJ11305 | 1.16 | 2.15 |
| 405646 | | | C12000200: gi\|4557225\|ref\|NP_000005.1\| al | 1.16 | 1.11 |
| 438438 | AA257992 | Hs.50651 | Janus kinase 1 (a protein tyrosine kinas | 1.16 | 1.08 |
| 431427 | AK000401 | Hs.252748 | Homo sapiens cDNA FLJ20394 fis, clone KA | 1.16 | 2.08 |
| 419885 | AA251561 | Hs.48689 | ESTs | 1.16 | 2.07 |
| 427679 | AA973904 | Hs.176092 | ESTs, Moderately similar to MYPH_HUMAN M | 1.16 | 2.15 |
| 443865 | AW296385 | Hs.146139 | hypothetical protein FLJ12610 | 1.15 | 2.05 |
| 415511 | AI732617 | Hs.182362 | ESTs | 1.15 | 3.44 |
| 417988 | AA210878 | Hs.111219 | ESTs, Moderately similar to ALU1_HUMAN A | 1.15 | 2.09 |
| 405058 | | | Target Exon | 1.15 | 1.16 |
| 446623 | AF279865 | Hs.15711 | kinesin family member 13B | 1.15 | 1.21 |
| 419754 | H52299 | Hs.308467 | Homo sapiens mRNA; cDNA DKFZp58610523 (f | 1.15 | 1.15 |
| 420003 | AA256906 | Hs.111364 | ESTs, Weakly similar to ubiquitous TPR m | 1.15 | 2.06 |
| 422988 | AW673847 | Hs.97321 | ESTs | 1.15 | 1.00 |
| 426371 | M63967 | Hs.169517 | aldehyde dehydrogenase 1 family, member | 1.15 | 2.31 |
| 422895 | NM_015958 | | CGI-30 protein | 1.15 | 2.08 |
| 426295 | AW367283 | Hs.278270 | zinc finger protein 6 (CMPX1) | 1.15 | 2.13 |
| 448323 | AI492298 | Hs.170915 | ESTs | 1.14 | 2.54 |
| 414244 | AA287801 | Hs.71711 | ESTs, Moderately similar to Z195_HUMAN Z | 1.14 | 2.23 |
| 442872 | AI471987 | Hs.173045 | ESTs | 1.14 | 2.09 |
| 425318 | AU076845 | Hs.155596 | BCL2/adenovirus E1B 19 kD-interacting pro | 1.14 | 2.33 |
| 415667 | F11582 | Hs.78582 | developmentally regulated GTP-binding pr | 1.14 | 1.10 |
| 401058 | | | Target Exon | 1.14 | 2.20 |
| 409838 | AW502928 | | gb: UI-HF-BP0p-aiw-e-10-0-UI.r1 NIH_MGC_5 | 1.14 | 2.15 |
| 438493 | AI130740 | Hs.6241 | phosphoinositide-3-kinase, regulatory su | 1.14 | 1.00 |
| 404392 | | | C7001460: gi\|12667420\|gb\|AAK01436.1\|AF332 | 1.14 | 2.82 |
| 433220 | AI076192 | Hs.131933 | ESTs | 1.14 | 2.78 |
| 405166 | | | Target Exon | 1.14 | 2.23 |
| 401038 | | | C11000425: gi\|4507721\|ref\|NP_003310.1\| ti | 1.14 | 2.71 |
| 414052 | AW578849 | Hs.283552 | ESTs, Weakly similar to unnamed protein | 1.14 | 2.08 |
| 442043 | BE567620 | Hs.99210 | ESTs | 1.13 | 2.17 |
| 419727 | AW160796 | Hs.92700 | DKFZP564O243 protein | 1.13 | 1.14 |
| 425206 | NM_002153 | Hs.155109 | hydroxysteroid (17-beta) dehydrogenase 2 | 1.13 | 2.07 |
| 414682 | AL021154 | Hs.76884 | inhibitor of DNA binding 3, dominant neg | 1.13 | 1.47 |
| 402712 | | | C1003562*: gi\|10047177\|dbj\|BAB13382.1\| (A | 1.13 | 1.18 |
| 452289 | BE568205 | Hs.28827 | mitogen-activated protein kinase kinase | 1.12 | 2.16 |
| 401496 | | | Target Exon | 1.12 | 1.10 |
| 459249 | AI970399 | Hs.240079 | ESTs | 1.12 | 2.67 |
| 447495 | AW401864 | Hs.18720 | programmed cell death 8 (apoptosis-induc | 1.12 | 2.03 |
| 428422 | AI557280 | Hs.184270 | capping protein (actin filament) muscle | 1.11 | 2.60 |
| 421762 | AA297546 | | gb: EST113074 Fetal brain III Homo sapien | 1.11 | 2.15 |
| 405855 | | | Target Exon | 1.11 | 1.98 |
| 428972 | AK001470 | Hs.194692 | cysteine desulfurase | 1.11 | 2.19 |
| 406761 | AI241715 | Hs.77039 | ATP synthase, H transporting, mitochondr | 1.10 | 3.33 |
| 432425 | AF070619 | Hs.274539 | Homo sapiens clone 24481 mRNA sequence | 1.10 | 2.30 |
| 446241 | AI004677 | Hs.179260 | chromosome 14 open reading frame 4 | 1.10 | 2.28 |
| 424454 | AB011139 | Hs.147946 | optic atrophy 1 (autosomal dominant) | 1.10 | 2.18 |
| 418242 | AW976183 | Hs.88414 | BTB and CNC homology 1, basic leucine zi | 1.10 | 2.07 |
| 437407 | AI479332 | Hs.129031 | ESTs | 1.10 | 2.09 |
| 447459 | AI380255 | Hs.159424 | ESTs | 1.10 | 2.22 |
| 426682 | AV660038 | Hs.2056 | UDP glyconyltransferase 1 family, polype | 1.09 | 2.33 |
| 403655 | | | NM_003071: Homo sapiens SWI/SNF related, | 1.09 | 2.25 |
| 433156 | R59206 | Hs.17519 | Homo sapiens cDNA: FLJ22539 fis, clone H | 1.09 | 2.70 |
| 403826 | | | Target Exon | 1.09 | 1.10 |
| 433333 | AI016521 | Hs.71816 | v-akt murine thymoma viral oncogene homo | 1.09 | 1.06 |
| 451382 | H86180 | Hs.221513 | ESTs | 1.08 | 2.75 |
| 454717 | AW815123 | | gb: QV4-ST0212-261199-045-b01 ST0212 Homo | 1.08 | 1.98 |
| 422743 | BE304678 | Hs.119598 | ribosomal protein L3 | 1.08 | 1.00 |
| 411672 | AJ275986 | Hs.71414 | transcription factor (SMIF gene) | 1.08 | 1.00 |
| 452748 | AB011128 | Hs.30512 | Homo sapiens mRNA for KIAA0556 protein, | 1.08 | 2.45 |
| 428330 | L22524 | Hs.2256 | matrix metalloproteinase 7 (matrilysin, | 1.08 | 2.15 |
| 447703 | AI420277 | | gb: tf06c12.x1 NCI_CGAP_Pr28 Homo sapiens | 1.08 | 2.05 |
| 452420 | BE564871 | Hs.29463 | centrin, EF-hand protein, 3 (CDC31 yeast | 1.07 | 2.03 |
| 455234 | R41084 | | gb: Hk763-f Adult heart, Clontech Homo sa | 1.07 | 2.08 |
| 413945 | NM_000591 | Hs.75627 | CD14 antigen | 1.07 | 0.91 |
| 417333 | AL157545 | Hs.173179 | bromodomain and PHD finger containing, 3 | 1.07 | 1.00 |
| 434105 | AW952124 | Hs.13094 | presenilins associated rhomboid-like pro | 1.07 | 1.11 |
| 455630 | AV655701 | Hs.75183 | cytochrome P450, subfamily IIE (ethanol- | 1.06 | 2.14 |
| 455424 | AW937733 | | gb: QV3-DT0045-210100-063-d06 DT0045 Homo | 1.05 | 2.03 |
| 438324 | AI792660 | Hs.6162 | KIAA0771 protein | 1.05 | 2.27 |
| 421604 | AW293880 | Hs.248367 | MEGF11 protein | 1.05 | 2.00 |
| 422614 | AI908006 | Hs.295362 | Homo sapiens cDNA FLJ14459 fis, clone HE | 1.04 | 2.33 |
| 404058 | | | Target Exon | 1.04 | 2.10 |
| 453085 | AW954243 | | KIAA0251 protein | 1.04 | 2.18 |
| 417500 | H59970 | | gb: yr16f04.r1 Soares fetal liver spleen | 1.04 | 2.40 |
| 408653 | AW410189 | Hs.98074 | itchy (mouse homolog) E3 ubiquitin prote | 1.04 | 2.73 |
| 440439 | N92818 | Hs.64754 | ESTs, Weakly similar to potential CDS [H | 1.04 | 2.05 |
| 409209 | AA460160 | Hs.73217 | ESTs | 1.04 | 2.73 |
| 456107 | AA160000 | Hs.137396 | ESTs, Weakly similar to JC5238 galactosy | 1.03 | 2.18 |
| 415403 | F07923 | Hs.26744 | ESTs | 1.02 | 2.43 |
| 455591 | BE008018 | | gb: QV0-BN0147-290400-214-c01 BN0147 Homo | 1.02 | 2.08 |
| 428491 | AF091035 | Hs.184627 | KIAA0118 protein | 1.02 | 2.81 |
| 407788 | BE514982 | Hs.38991 | S100 calcium-binding protein A2 | 1.02 | 2.11 |
| 434540 | NM_016045 | Hs.3945 | CGI-107 protein | 1.02 | 2.78 |
| 442174 | AI690080 | Hs.128907 | ESTs, Weakly similar to ARIX homeodomain | 1.02 | 2.05 |
| 413431 | AW246428 | Hs.75355 | ubiquitin-conjugating enzyme E2N (homolo | 1.02 | 1.00 |
| 452436 | BE077546 | Hs.31447 | ESTs, Moderately similar to A46010 X-lin | 1.02 | 2.25 |
| 454412 | AW582568 | | gb: RC1-ST0278-080100-011-h04 ST0278 Homo | 1.00 | 2.20 |
| 426955 | AA393669 | Hs.238094 | ESTs | 1.00 | 2.18 |
| 425910 | AA830797 | Hs.184760 | CCAAT-box-binding transcription factor | 1.00 | 2.10 |
| 405710 | | | CX000682: gi\|12741327\|ref\|XP_008833.2\| zi | 1.00 | 2.00 |
| 400296 | AA305627 | Hs.139336 | ATP-binding cassette, sub-family C (CFTR | 1.00 | 1.00 |
| 402001 | | | Target Exon | 1.00 | 1.00 |
| 402812 | | | NM_004930*: Homo sapiens capping protein | 1.00 | 1.00 |
| 402892 | | | Target Exon | 1.00 | 1.00 |
| 403329 | | | Target Exon | 1.00 | 1.00 |
| 407202 | N58172 | Hs.109370 | ESTs | 1.00 | 1.00 |
| 408684 | R61377 | Hs.12727 | hypothetical protein FLJ21610 | 1.00 | 1.00 |
| 410555 | U92649 | Hs.64311 | a disintegrin and metalloproteinase doma | 1.00 | 1.00 |
| 413573 | AI733859 | Hs.149089 | ESTs | 1.00 | 1.00 |
| 414343 | AL036166 | Hs.323378 | coated vesicle membrane protein | 1.00 | 1.00 |
| 414422 | AA147224 | Hs.249195 | Homeo box A13 | 1.00 | 1.00 |
| 417006 | AW673606 | Hs.80758 | aspartyl-tRNA synthetase | 1.00 | 1.00 |
| 421577 | BE465451 | Hs.105925 | single-minded (Drosophila) homolog 1 | 1.00 | 1.00 |
| 423349 | AF010258 | Hs.127428 | homeo box A9 | 1.00 | 1.00 |
| 424273 | W40460 | Hs.144442 | phospholipase A2, group X | 1.00 | 1.00 |
| 424649 | BE242035 | Hs.151461 | embryonic ectoderm development | 1.00 | 1.00 |
| 426827 | AW067805 | Hs.172665 | methylenetetrahydrofolate dehydrogenase | 1.00 | 1.00 |
| 427308 | D26067 | Hs.174905 | KIAA0033 protein | 1.00 | 1.00 |
| 429597 | NM_003816 | Hs.2442 | a disintegrin and metalloproteinase doma | 1.00 | 1.00 |
| 430261 | AA305127 | Hs.237225 | hypothetical protein HT023 | 1.00 | 1.00 |
| 431078 | U82827 | Hs.249195 | homeo box A13 | 1.00 | 1.00 |
| 433222 | AW514472 | Hs.238415 | dickkopf (Xenopus laevis) homolog 4 | 1.00 | 1.00 |
| 434980 | AW770553 | Hs.14553 | sterol O-acyltransferase (acyl-Coenzyme | 1.00 | 1.00 |
| 435974 | U29690 | Hs.37744 | Homo sapiens beta-1 adrenergic receptor | 1.00 | 1.00 |
| 443054 | AI745185 | Hs.8939 | yes-associated protein 65 kDa | 1.00 | 1.00 |
| 443564 | AI921685 | Hs.199713 | ESTs | 1.00 | 1.00 |
| 444542 | AI161293 | Hs.280380 | aminopeptidase | 1.00 | 1.00 |
| 445413 | AA151342 | Hs.12677 | CGI-147 protein | 1.00 | 1.00 |
| 448706 | AW291095 | Hs.21814 | interleukin 20 receptor, alpha | 1.00 | 1.00 |
| 448807 | AI571940 | Hs.7549 | ESTs | 1.00 | 1.00 |
| 449448 | D60730 | Hs.57471 | ESTs | 1.00 | 1.00 |
| 449517 | AW500106 | Hs.23643 | serine/threonine protein kinase MASK | 1.00 | 1.00 |
| 450568 | AL050078 | Hs.25159 | Homo sapiens cDNA FLJ10784 fis, clone NT | 1.00 | 1.00 |
| 451844 | T61430 | | gb: yc06a03.s1 Stratagene lung (937210) H | 1.00 | 1.00 |
| 452039 | AI922988 | Hs.172510 | ESTs | 1.00 | 1.00 |
| 452795 | AW392555 | Hs.18878 | hypothetical protein FLJ21620 | 1.00 | 1.00 |
| 453096 | AW294631 | Hs.11325 | ESTs | 1.00 | 1.00 |
| 453370 | AI470523 | Hs.139336 | ATP-binding cassette, sub-family C (CFTR | 1.00 | 1.00 |
| 453966 | BE148734 | Hs.63325 | transmembrane protease, serine 4 | 1.00 | 1.00 |
| 405580 | | | Target Exon | 1.00 | 1.00 |
| 430268 | AK000737 | Hs.237480 | hypothetical protein FLJ20730 | 1.00 | 1.00 |
| 450377 | AB033091 | Hs.74313 | KIAA1265 protein | 1.00 | 1.00 |
| 433226 | AW503733 | Hs.9414 | KIAA1488 protein | 1.00 | 1.00 |
| 412719 | AW016610 | Hs.816 | ESTs | 1.00 | 1.00 |
| 425289 | AW139342 | Hs.155530 | interferon, gamma-inducible protein 16 | 1.00 | 1.00 |
| 446921 | AB012113 | Hs.16530 | small inducible cytokine subfamily A (Cy | 1.00 | 1.00 |
| 439706 | AW872527 | Hs.59761 | ESTs, Weakly similar to DAP1_HUMAN DEATH | 1.00 | 1.00 |
| 438817 | AI023799 | Hs.163242 | ESTs | 1.00 | 1.00 |
| 455474 | AW948094 | | gb: RC0-MT0012-290300-031-c10 MT0012 Homo | 0.99 | 2.00 |
| 420148 | U34227 | Hs.95361 | myosin VIIA (Usher syndrome 1B (autosoma | 0.99 | 2.33 |
| 428466 | AF151063 | Hs.184456 | hypothetical protein | 0.98 | 2.20 |
| 421594 | R45689 | Hs.21889 | Homo sapiens cDNA FLJ12978 fis, clone NT | 0.98 | 3.09 |
| 444654 | AV650572 | Hs.23440 | KIAA1105 protein | 0.98 | 2.00 |
| 409759 | N40285 | Hs.81182 | histamine N-methyltransferase | 0.97 | 2.20 |
| 401936 | | | Target Exon | 0.97 | 2.39 |
| 403463 | | | Target Exon | 0.96 | 2.58 |
| 434421 | AI915927 | Hs.34771 | ESTs | 0.96 | 2.15 |
| 412636 | NM_004415 | | desmoplakin (DPI, DPII) | 0.95 | 2.01 |
| 442432 | BE093589 | Hs.38178 | hypothetical protein FLJ23468 | 0.93 | 2.55 |
| 421938 | AA405951 | | gb: zu66c01.r1 Soares_testis_NHT Homo sap | 0.93 | 3.10 |
| 447470 | BE618324 | Hs.263561 | ESTs, Weakly similar to A53531 oncofetal | 0.92 | 2.08 |
| 448369 | AW268962 | Hs.111335 | ESTs | 0.91 | 2.35 |
| 421710 | AB007930 | Hs.107088 | KIAA0461 protein | 0.91 | 2.63 |
| 406805 | AI686003 | Hs.296031 | ESTs | 0.91 | 2.21 |
| 447475 | AI380797 | Hs.158992 | ESTs | 0.90 | 3.25 |
| 428892 | U82828 | Hs.194382 | ataxia telangiectasia mutated (includes | 0.90 | 2.02 |
| 450222 | U75308 | Hs.24644 | TATA box binding protein (TBP)-associate | 0.89 | 2.73 |
| 401572 | | | C15001384*: gi\|12737057\|ref\|XP_012129.1\| | 0.88 | 2.00 |
| 429226 | AA913330 | Hs.53542 | choreoacanthocytosis gene; KIAA0986 prot | 0.88 | 2.37 |
| 421979 | AW062518 | Hs.233150 | hypothetical protein MGC5560 | 0.87 | 3.70 |
| 407614 | NM_001932 | Hs.37144 | membrane protein, palmitoylated 3 (MAGUK | 0.86 | 2.02 |
| 417912 | R25269 | Hs.50547 | ESTs | 0.86 | 2.00 |
| 401654 | | | NM_007242: Homo sapiens DEAD/H (Asp-Glu-A | 0.86 | 2.11 |
| 403149 | | | NM_001450: Homo sapiens four and a half L | 0.86 | 2.19 |
| 413000 | BE046280 | | gb: hn43c09.x2 NCI_CGAP_RDF2 Homo sapiens | 0.85 | 2.40 |
| 425166 | AK001456 | Hs.154919 | KIAA0625 protein | 0.85 | 2.18 |
| 447371 | AA334274 | Hs.18368 | DKFZP564B0769 protein | 0.84 | 2.13 |
| 452801 | AI935587 | Hs.34447 | ESTs | 0.84 | 2.55 |
| 400957 | | | Target Exon | 0.83 | 2.15 |
| 426420 | BE383808 | Hs.322430 | NDRG family, member 4 | 0.83 | 2.14 |
| 429354 | AA451666 | Hs.269363 | ESTs | 0.80 | 2.25 |
| 417831 | H16423 | Hs.82685 | CD47 antigen (Rh-related antigen, integr | 0.78 | 2.43 |
| 443368 | BE568891 | Hs.199210 | ESTs, Moderately similar to bK116F5.2[H | 0.78 | 2.00 |
| 441901 | AI914445 | Hs.128103 | ESTs | 0.76 | 2.06 |
| 429462 | AI890356 | Hs.127804 | Homo sapiens, clone IMAGE: 3536432, mRNA, | 0.76 | 2.03 |
| 403010 | | | C21000152: gi\|6226483\|sp\|Q52118\|YMO3_ERWS | 0.75 | 2.43 |
| 420344 | BE463721 | Hs.97101 | putative G protein-coupled receptor | 0.75 | 2.42 |
| 448332 | AW293110 | Hs.171068 | ESTs | 0.74 | 2.00 |
| 421674 | T10707 | Hs.296355 | hypothetical protein FLJ23138 | 0.71 | 2.18 |
| 450645 | AL117441 | Hs.301997 | hypothetical protein FLJ13033 | 0.67 | 2.06 |
| 448514 | AB020626 | Hs.301866 | KIAA0819 protein | 0.67 | 2.03 |
| 431609 | AW792792 | Hs.264330 | N-acylsphingosine amidohydrolase (acid c | 0.60 | 2.54 |
| 417512 | X76534 | Hs.82226 | glycoprotein (transmembrane) nmb | 0.60 | 2.00 |
| 425960 | AW410646 | Hs.164649 | hypothetical protein DKFZp434H247 | 0.57 | 2.15 |
| 408077 | AL133574 | Hs.42458 | Homo sapiens mRNA; cDNA DKFZp586C1817 (f | 0.53 | 2.29 |

Pkey: Unique Eos probeset identifier number
ExAccn: Exemplar Accession number, Genbank accession number
UnigeneID: Unigene number
Unigene Title: Unigene gene title
R1 80th percentile of Ta or T1 tumor Als from patients who did not upstage divided by the 80th percentile of Ta or T1 tumor Als from patients who did upstage
R2 median of Ta or T1 tumor Als from patients who did not upstage divided by the median of Ta or T1 tumor Als from patients who upstaged

TABLE 9B

| Pkey | CAT Number | Accession |
|---|---|---|
| 409345 | 112147_1 | AI949109 AW118631 AI833148 AW117891 R45932 R80970 AA733110 AW269428 T27024 |
| | | R97693 AI149202 AI475492 H98845 AA609665 AI125598 AW663742 AI140029 AW103164 AA126862 N66829 W65398 |
| | | H94509 W65300 R07712 R36955 AA812477 AA609576 AI754304 Z41075 AA970004 AW274661 AA923584 AI673108 |
| | | AA070706 AA541812 T90938 |
| 409540 | 1138613_1 | AW409569 BE297044 BE295828 |
| 409806 | 1155259_1 | AW500960 AW501280 AW500814 |
| 409838 | 1155987_1 | AW502928 AW505606 AW501576 AW501577 |
| 409844 | 1156139_1 | AW502336 AW502339 AW501736 AW501839 |
| 409855 | 1156256_1 | AW502461 AW503000 AW502207 AW501862 |
| 409865 | 1156518_1 | AW502208 AW502366 AW502148 |
| 410600 | 121108_1 | AW575742 BE549623 AI335824 BE463447 AA729043 AW408712 AW499616 AA086179 |
| | | AW499617 AA191322 |
| 410846 | 1223902_1 | AW807057 AW807054 AW807189 AW807193 AW807369 AW807429 AW807364 |
| | | AW807365 AW807078 AW807256 AW807180 AW807331 |
| 410946 | 1227589_1 | AW811502 AW811521 AW811548 AW811471 AW811511 AW811508 |
| 411456 | 1246706_1 | AW847588 AW847716 AW847664 AW847592 |
| 411609 | 1251530_1 | AW993680 AW853769 |
| 412210 | 1283615_1 | AW901492 AW947725 AW901448 |
| 412383 | 1292509_1 | AW947577 AW947574 AW947576 AW947734 AW947733 AW947732 |
| 412560 | 130601_1 | R24601 R23657 AA194467 AI948584 AI678666 AA194383 AI765219 AA702993 AA813511 |
| | | AA620965 AI990303 AI624882 AI003925 AI338870 AI004689 AI004690 AI127228 Z25302 F29302 BE044308 F32992 |
| | | AA112966 T30825 F24958 F18071 C00537 T30841 R24502 AI934786 AI770075 AI144132 AA812597 AW203978 |
| | | H82735 AI813349 AI1142908 C04894 AI208243 AI208044 N89963 AI767866 AI290470 AI865963 H83611 BE463806 |
| | | AI082639 AA507760 |
| 412636 | 13165_1 | NM_004415 AL031058 M77830 BE149760 AW752599 AW848723 AW376697 |
| | | AW376817 AW376699 AW848371 AW376782 AW848789 AW361413 AW849074 AW997139 AW799304 AW799309 |
| | | BE077020 BE077017 BE185187 AW997196 BE156621 BE179915 BE006561 BE143155 AW890985 BE002107 |
| | | AW103521 AA857316 AW383133 BE011378 AW170253 BE185750 AW886475 BE160433 J05211 BE082576 |
| | | BE082584 BE004047 AW607238 AW377700 AW377699 BE082526 BE082505 BE082507 BE082514 AW178000 |
| | | AW177933 AI905935 AW747877 AW748114 BE148516 AW265328 AW847678 AW847688 AW365151 AW365148 |
| | | AW365153 AW365156 AW365175 AW365157 AW365154 AW068840 BE005272 AW365145 BE001925 BE182166 |
| | | BE144243 BE001923 AI951766 AI434518 BE184920 BE184933 AI284090 BE184941 AW804674 BE184924 |
| | | C04715 W39488 AW995615 BE184948 BE159646 AW606653 AA099891 AA131128 AA337270 AA340777 |
| | | AW384371 AA852212 R58704 AW366566 AW364859 AA025851 AA025852 AA455100 AA719958 AW352220 |
| | | AW996245 BE165351 BE073467 AA377127 AW890264 AW609750 AW391912 AW849690 T87267 AW853812 |
| | | AA852213 W74149 BE009090 AA056401 H91011 AW368529 AW390272 C18467 AW674920 N57176 AA026480 |
| | | AW576767 H93284 AA026863 AW177787 AA026654 AW177786 BE092134 BE092137 BE092136 AW177784 |
| | | AI022862 BE091653 AW376811 AW848592 AA040018 BE185331 BE182164 AA368564 AW951576 T29918 |
| | | AA131077 W95048 W25458 AW205789 H90899 N29754 W32490 R20904 BE167181 BE167165 N84767 |
| | | H27408 H30146 AI190590 C03378 AI554403 AI205263 AA128470 AI392926 AF139065 AW370813 AW370827 |
| | | AW798417 AW798780 AW798883 AW798569 R33557 AA149190 C03029 AW177783 AA088866 AW370829 |
| | | AA247685 BE002273 AI760816 AI439101 AW879451 AI700963 AA451923 AI340326 AI590975 T48793 AI568096 |
| | | AI142882 AA039975 AI470146 AA946936 BE067737 BE067786 W19287 AA644381 AA702424 AI417612 AI306554 |
| | | AI686869 AI568892 AW190555 AI571075 AI220573 AA056527 AI471874 AI304772 AW517828 AI915596 AI627383 |
| | | AI270345 AW021347 AW166807 AW105614 AI346078 AA552300 W95070 AI494069 AI911702 AA149191 AA026864 |
| | | AI830049 AI887258 AW780435 AI910434 AI819984 AI858282 AI078449 AI025932 AI860584 AI635878 AA026047 |
| | | AA703232 D12062 AW192085 AA658154 AW514597 AW591892 T87181 AA782066 AW243815 AW150038 |
| | | AW268383 AW004633 AI927207 AA782109 AW473233 AI804485 AW169216 AI572669 AA602182 AW015480 |
| | | AW771865 AI270027 AA961816 AA283207 AI076962 AI498487 AI348053 AI783914 H44405 AW799118 AA128330 |
| | | AA515500 AA918281 W02156 AI905927 AA022701 W38382 R20795 T77861 AW860878 |
| 412998 | 1343218_1 | BE046254 BE046673 BE046253 |
| 413000 | 1343239_1 | BE046280 BE046763 BE046676 |
| 413611 | 1380017_1 | BE153275 BE153189 BE153329 BE153022 BE153030 BE152974 |
| 413709 | 1384144_1 | BE158687 BE158688 |
| 413804 | 1390710_1 | T64682 BE168190 BE168256 |
| 413895 | 1397743_1 | BE178160 BE177986 BE178330 BE178480 |
| 415789 | 1555357_1 | H01581 H12850 R65905 H13053 |
| 416023 | 156696_1 | AA173029 BE467711 AA176710 |
| 416272 | 158407_1 | AA178882 AA179898 AA178897 |
| 417500 | 168443_1 | H59970 AA203382 R08822 |
| 419555 | 185884_1 | AA244416 AA244401 |
| 420685 | 195591_1 | AA279362 AA454496 AA584871 |
| 421762 | 206590_1 | AA297546 AA297410 AA297401 AA297465 AA297268 AW966174 |
| 421938 | 209376_1 | AA405951 AA300675 AA412243 AA412383 |
| 422895 | 22276_1 | NM_015958 AF132964 AA088658 N28882 AI197842 AA338679 AA405666 W16871 |
| | | AA385447 AI928315 AI928318 AW846613 AW960009 AI860687 AI206534 AI961336 T64873 AI735559 H58826 |
| | | AA857710 AW337576 BE242131 AA339340 AA371380 AA334618 AA316398 R96579 AW771996 AA366110 W07461 |
| | | H77948 |
| 422984 | 223488_3 | W28614 W27435 AI983043 AA364395 AW572472 AW190386 AI129278 AI913081 AW473549 |
| | | AA830713 AI982871 AI638647 AI828466 AW572486 N52583 N89687 AW075567 AI571047 AI887479 AI559469 |
| | | AI685802 AA805256 AI458777 AA974369 AI866929 AI886032 AI823925 AI823566 AW198135 AI287510 |
| | | AI565910 AA765775 AI866019 AI263697 AI355825 R42668 AA894603 AW105585 AI824555 |
| | | AW339175 D20479 |
| 432638 | 35173_4 | AI017717 AA349996 AA350286 BE501310 R48207 AI372769 AJ003450 AA915976 AI261513 |
| | | AI033019 AI222825 AW150983 AA420700 AA420670 AW884784 Z40157 |
| 433821 | 374566_1 | AW182416 AA918195 AA778707 AA927922 AA868718 AA853991 AA609856 AA634398 |
| 434767 | 39297_1 | AF153201 AW888811 AW888810 AW842970 AA383181 F35832 F26805 |
| | | X78930 AF026094 N83362 AA206766 AW874294 AA284205 AI091885 AA206800 AW370684 |
| | | AA904608 AA806352 AA894757 AW068376 AA807284 AA311341 AW958314 |
| 434809 | 393566_1 | AW974687 AA649656 AA652145 |
| 438129 | 450500_1 | AA778647 AW070999 R42302 |
| 438713 | 463722_1 | H16902 AA814856 T65225 AI523569 F09869 F04737 AI538427 AI051714 AW172603 F02602 |
| | | F01437 AI684417 AI801950 F02970 Z39367 F02882 T16704 H14597 AW952332 R52080 H10427 T06175 |
| | | T31057 AI381431 R60918 R41255 R43059 R39905 H19330 R36968 |
| 438924 | 4669_1 | BE535511 M62098 AA306787 AW891766 AA348998 AA338869 AA344013 AW956561 AW389343 |
| | | AW403607 L40391 AW408435 AA121738 AI568978 H13317 R20373 AW948724 AW948744 AA335023 |
| | | AA436722 AA448690 C21404 AW884390 AA345454 AA303292 AA174174 BE092290 T90614 AA035104 |
| | | R76028 AA126924 AA741086 AW022056 AW118940 AA121666 AI832409 AA683475 AI140901 AI623576 |
| | | AW519064 AW474125 AI953923 AI735349 AW150109 AI436154 AW118130 AW270782 AI804073 N27434 |
| | | AA876543 AA937815 AI051166 AA505378 AI041975 AI335355 AI089540 AA662243 AI127912 AI925604 |
| | | AI250880 AI366874 AI564386 AI815196 AI683526 AI435885 AI160934 H79030 AI801493 |
| | | AA448691 AI673767 AI076042 AI804327 AA813438 AA680002 AI274492 T16177 AI287337 AI935050 |
| | | AA907805 AA911493 AI589411 AI371358 AW576236 AI078866 AW516168 AA346372 AI560185 |
| | | AA471009 R75857 AA296025 AA523155 AA853168 AI696593 AI658482 AI566601 AW072797 |
| | | AA128047 AA035502 AW243274 AA992517 R43760 |
| 439004 | 467743_1 | AW979062 AA848000 AA847968 AA829138 |
| 441623 | 52182_1 | AA315805 AA376906 BE539395 AW579186 H44349 BE328145 BE041644 AW579187 AW366504 |
| | | H28241 H25318 W37573 AW600919 AA528775 R75904 AW780125 AA149420 AI672414 AI122760 |
| | | AA345675 AW172758 AI337912 AI961291 AW090300 AI090244 BE219837 AI623661 BE501576 |
| | | AI742232 AW887496 BE501734 AI023964 AI288904 AA975373 AA890325 AI458424 AI984583 |
| | | N32562 AI358102 AW241694 AI038448 AA576391 AI018389 AI672071 AA977874 |
| | | W37448 AW189392 AA612894 AI373653 H89551 H89365 AI699774 AI277548 |
| 442473 | 543413_1 | W27992 AF056988 |
| 442506 | 54405_1 | BE566411 AL121194 AW976385 AW366882 AI767324 |
| 445182 | 632151_1 | AW189787 AI215430 AW268499 AW205930 AI392907 BE093017 BE093019 BE093010 |
| 447703 | 733191_1 | AI420277 AW747989 W26565 |
| 448079 | 74834_1 | R76981 AA101801 AA101722 AA122297 AA098802 BE006483 AA343200 AW963375 BE090425 |
| | | AA213871 BE621402 AA309094 AW960171 AI275045 Z44230 AW243724 AI051487 AI376624 |
| | | R68631 AW978550 C00116 AI984051 AA122265 AI379941 AW205843 AW205839 AW190219 |
| | | AW129532 AI954133 AI668869 AI383948 AI537386 AA213788 AI088416 AI360826 AA101802 |
| | | AI619505 AA101795 AA101723 AW614392 R64287 AA927599 AI251904 AI803003 AI675123 |
| | | AW023079 AW134959 AI926156 AA831069 AI638324 F29901 R46085 F03383 C17731 R20584 |
| | | T90131 |
| 450936 | 85190_2 | AI033745 AI034133 AA844424 AW166024 AI831699 AI971097 AA011685 H70852 H70851 |
| 451844 | 888230_1 | T61430 AI820546 AI821336 |
| 452900 | 93691_1 | AA626794 AA626779 AA071274 AA928041 AI954235 N71035 N70230 AW674412 AI871136 AI563955 |
| | | AI954237 AA649543 AI340231 AI368586 AI868721 W44486 T83736 AA126250 AI343619 H94297 |
| | | T47633 AI672897 AA496355 R23240 AI814680 AA902119 AA644262 N67040 AW074273 AI357512 |
| | | AA865354 AI027942 R33837 H95828 N63928 AI418701 AI186469 AA693672 AA778429 AA128352 |
| | | AW954072 C00015 AA861853 AW022016 AI955645 AI753118 AI755095 AA029523 T70086 |
| | | AA029458 AW675640 N79606 AI659597 AI417119 AI804089 AI383091 AA128493 T83907 T47632 |
| | | AA094118 AA496405 H94191 AW887759 N98454 AA512988 AI623761 AW028373 AW249740 |
| | | AI187029 AA991733 AI683085 AI336036 D60550 D60466 D60009 D60248 AW262673 AI524080 |
| | | AA205643 AI969141 AW674989 AA093704 R81248 R73069 W01019 R23316 C14688 |
| | | W44485 AA093663 T70157 H57883 R94790 D81287 D81788 AA364340 AA383533 |
| | | N41706 BE295144 D60973 D81466 D60551 R33836 D60249 AA354560 W01933 T71478 |
| | | AA515461 F27984 BE084745 W19084 R81247 AI080252 AI382863 AA205642 |
| 453085 | 94851_1 | AW954243 AA829930 AA412478 AA828434 AA814538 AI927418 AI192435 W52897 AA443666 AA031913 |
| | | AI683306 AA918481 AI183314 D83907 AI206832 AA876122 D83836 D83838 D82533 |
| | | AI761290 AI191125 AI143749 AW771909 AI241436 AI767267 W56507 AA847787 |
| | | AA568692 T10502 AI247870 AA715017 AA643304 AA890233 AA811387 AA897470 |
| | | AA907729 AI708679 AI078010 AA452830 AW419160 AI783713 N80205 W56778 AA676899 |
| | | AI888718 N69930 AI338935 AI217580 AA639508 AA575836 BE046852 AI312651 AI038406 |
| | | AA628649 AA643838 AI493761 AA032024 W38849 AA340178 AA447052 AA452969 W19369 |
| | | AA296364 H44229 W58767 C05751 C05835 AI741989 N98532 AW102617 AA412583 |
| | | AI922246 W38495 AA355375 AA928571 C06275 AA352500 N93132 |
| 454412 | 1174764_1 | AW582568 AW818656 AW818647 AW818655 AW818637 AW818234 |
| 454555 | 1223870_1 | AW807095 AW807022 AW845880 AW807096 AW807461 AW846116 AW807070 |
| 454717 | 1230516_1 | AW815123 AW815138 AW815259 |
| 455234 | 1265385_1 | R41084 AW875856 |
| 455424 | 1289247_1 | AW937733 AW937727 AW937883 |
| 455474 | 1292960_1 | AW948094 AW948118 AW948070 AW948093 AW948084 AW948088 AW948074 AW948079 AW948117 |
| | | AW948085 AW948081 AW948096 AW948102 AW948087 AW948080 AW948061 AW948091 AW948098 |
| | | AW948089 AW948104 AW948119 AW948069 AW948100 AW948112 AW948121 AW948068 AW948109 |
| | | AW948105 AW948097 AW948120 AW948108 AW948111 AW948114 AW948115 AW948072 AW948083 |
| | | AW948095 AW948116 AW948078 AW948077 AW948071 AW948067 AW948107 AW948122 AW948065 |
| | | AW948064 AW948113 AW948063 AW948062 |
| 455476 | 1293055_1 | AW948172 AW948178 AW948169 AW948176 AW948191 AW948192 AW948186 AW948184 AW948187 |
| | | AW948188 AW948189 AW948181 AW948177 AW948171 AW948183 AW948173 |
| 455591 | 1335166_1 | BE008018 BE008025 BE008026 BE007959 BE007994 BE008016 BE008019 BE008024 BE008022 |
| | | BE008027 BE008029 BE008020 BE008015 BE008021 BE008028 BE008023 BE008030 BE008014 |
| 455604 | 1337197_1 | BE011183 BE011170 BE011333 BE011188 BE011181 BE011324 BE011161 BE011169 |
| 455842 | 1374629_1 | BE145837 BE145894 |
| 457747 | 397222_1 | AW975000 AA658945 AA661558 |
| 458991 | 850804_1 | AI743502 AI807438 |
| 459192 | 923891_1 | AW176180 AW176212 AI909464 AW176218 AW176171 AW176203 AW176181 AW176213 |

Pkey: Unique Eos probeset identifier number
CAT number: Gene cluster number
Accession: Genbank accession numbers

TABLE 9C

| Pkey | Ref | Strand | Nt_position |
|---|---|---|---|
| 400634 | 8567750 | Minus | 101102-101223, 101886-102018 |
| 400750 | 8119067 | Plus | 198991-199168, 199316-199548 |
| 400752 | 7331445 | Minus | 36215-36461 |
| 400772 | 8131629 | Minus | 34896-35021, 41078-41197 |
| 400773 | 8131629 | Minus | 44116-44238, 48208-48321 |
| 400788 | 7342055 | Plus | 184369-184715 |
| 400789 | 8307741 | Plus | 82281-83693 |
| 400835 | 8954121 | Plus | 89366-89622 |
| 400843 | 9188605 | Plus | 5863-5970, 7653-7784, 8892-9023, 9673-9807, 10634-10789, 15254-15403, 23827-23958 |
| 400844 | 9188605 | Plus | 24746-24872, 25035-25204 |
| 400845 | 9188605 | Plus | 34428-34612 |
| 400846 | 9188605 | Plus | 39310-39474 |
| 400847 | 9188605 | Plus | 44643-44835 |
| 400957 | 7705148 | Minus | 66959-67241 |
| 400967 | 7770682 | Minus | 32697-32999 |
| 401038 | 7232177 | Minus | 4277-4469 |
| 401041 | 7232177 | Plus | 44750-45076 |
| 401058 | 8117654 | Minus | 45226-45414 |
| 401155 | 9438289 | Plus | 31381-31526 |
| 401177 | 9438503 | Minus | 62773-63330 |
| 401196 | 9719673 | Plus | 33138-33834 |
| 401204 | 9743388 | Minus | 33694-33872 |
| 401286 | 9801342 | Minus | 147036-147318 |
| 401346 | 9926605 | Minus | 12031-13032 |
| 401371 | 9650602 | Plus | 80901-81283 |
| 401383 | 6721135 | Minus | 155543-157381 |
| 401496 | 7381769 | Minus | 82790-83002 |
| 401512 | 7622346 | Plus | 136399-136557 |
| 401563 | 8247910 | Plus | 91395-91763 |
| 401572 | 8570271 | Minus | 78651-78889 |
| 401593 | 7230957 | Plus | 10368-10572, 11293-12356 |
| 401600 | 4388746 | Minus | 27363-27518, 28727-28891, 29526-29731 |
| 401603 | 7689963 | Minus | 116659-116780 |
| 401613 | 4878062 | Plus | 22461-22831 |
| 401654 | 9097132 | Minus | 64695-64797 |
| 401660 | 9100664 | Minus | 173662-174024 |
| 401720 | 6468551 | Plus | 7783-8468 |
| 401784 | 7249190 | Plus | 148362-148606, 149453-149535, 149731-149962 |
| 401835 | 7139700 | Plus | 142257-142742 |
| 401886 | 7229913 | Minus | 79215-79393 |
| 401936 | 3808091 | Plus | 46817-46943 |
| 402001 | 9501818 | Plus | 68052-68223 |
| 402087 | 8117546 | Plus | 137069-137213, 138678-138828, 138969-139050 |
| 402138 | 7704985 | Plus | 14173-15108 |
| 402144 | 7242326 | Plus | 115425-115977 |
| 402190 | 8576067 | Minus | 76488-76959 |
| 402191 | 8576073 | Minus | 69410-69583 |
| 402329 | 7798735 | Plus | 15833-16180, 18419-18715, 22507-22624 |
| 402368 | 9558577 | Minus | 47218-47330, 48052-48203 |
| 402371 | 9558584 | Plus | 68736-68956 |
| 402378 | 9625333 | Minus | 41312-41468, 48313-48720 |
| 402449 | 9796674 | Plus | 59867-60039, 62588-62828, 63465-63623, 64923-65108 |
| 402474 | 7547175 | Minus | 53526-53628, 55755-55920, 57530-57757 |
| 402517 | 9798106 | Plus | 17569-17721 |
| 402599 | 7239666 | Plus | 5835-5987 |
| 402617 | 9930797 | Minus | 69466-69945 |
| 402712 | 8969253 | Minus | 10941-11138 |
| 402731 | 9211639 | Minus | 117913-118004, 121110-121211, 121327-121457, 125478-125623, 126540-126663 |
| 402737 | 9212184 | Minus | 13358-13552 |
| 402754 | 9213730 | Plus | 15345-15852 |
| 402760 | 9213869 | Plus | 136829-136952, 137336-137521 |
| 402812 | 6010110 | Plus | 25026-25091, 25844-25920 |
| 402845 | 9369286 | Plus | 160451-160617, 160788-161009 |
| 402869 | 6434643 | Minus | 138639-139335 |
| 402892 | 8086844 | Minus | 194384-194645 |
| 403010 | 3132346 | Plus | 78385-79052 |
| 403149 | 9799833 | Plus | 25034-25185 |
| 403326 | 8440025 | Minus | 110959-111122 |
| 403329 | 8516120 | Plus | 96450-96598 |
| 403342 | 7233487 | Minus | 42312-43750 |
| 403349 | 8569773 | Minus | 167815-168374 |
| 403381 | 9438267 | Minus | 26009-26178 |
| 403463 | 9929538 | Plus | 102596-102879 |
| 403655 | 8736093 | Plus | 65668-65859 |
| 403725 | 7534031 | Plus | 86737-86843 |
| 403728 | 7534291 | Minus | 34481-34671 |
| 403807 | 8439933 | Minus | 162963-165773 |
| 403826 | 9838209 | Plus | 121197-121358 |
| 403885 | 7710403 | Minus | 53259-53524 |
| 403943 | 7711864 | Plus | 100742-100904, 101322-101503 |
| 403988 | 8576087 | Plus | 16251-16462 |
| 404029 | 7671252 | Plus | 108716-111112 |
| 404058 | 3548785 | Plus | 99397-101808 |
| 404069 | 3168619 | Plus | 47310-47450 |
| 404178 | 7630978 | Minus | 178075-178383 |
| 404204 | 3169112 | Minus | 79868-80321 |
| 404231 | 8218035 | Minus | 61077-61322 |
| 404250 | 9187145 | Minus | 36099-36212, 37928-38075 |
| 404273 | 9885189 | Plus | 97789-98285, 99601-99855 |
| 404343 | 9838093 | Plus | 122664-122931 |
| 404349 | 7630858 | Minus | 61006-61187 |
| 404391 | 3135305 | Minus | 26030-26173, 27852-27997 |
| 404392 | 3135305 | Minus | 29738-29857 |
| 404397 | 9558608 | Minus | 104042-104232 |
| 404438 | 6984205 | Plus | 63413-63553 |
| 404439 | 7139680 | Plus | 55316-55585 |
| 404530 | 6479107 | Plus | 3157-3304 |
| 404596 | 9958262 | Minus | 104807-105043 |
| 404649 | 9796926 | Minus | 100027-100399 |
| 404666 | 7272179 | Minus | 18677-18993 |
| 404687 | 9797554 | Minus | 128456-128565 |
| 404744 | 9187237 | Plus | 71776-71852, 72885-73019, 73700-73822, 74692-74850 |
| 404751 | 7630939 | Plus | 113799-114252, 114393-114715 |
| 404769 | 8099713 | Minus | 175801-176823 |
| 404790 | 7230958 | Plus | 38611-38761 |
| 404906 | 7331453 | Minus | 100985-101126 |
| 404956 | 7387343 | Plus | 55883-56203 |
| 404977 | 3738341 | Minus | 43081-43229 |
| 405000 | 6957544 | Minus | 88854-89993 |
| 405022 | 7330304 | Plus | 217163-217439 |
| 405053 | 7651944 | Minus | 157134-157430 |
| 405058 | 7655685 | Plus | 150740-151556 |
| 405155 | 9966228 | Plus | 130469-130723 |
| 405163 | 9966267 | Minus | 161171-161299 |
| 405165 | 9966302 | Plus | 6461-6845 |
| 405166 | 9966302 | Plus | 40526-40891 |
| 405172 | 9966752 | Plus | 153027-153262 |
| 405180 | 7139743 | Plus | 65438-65740 |
| 405193 | 7230072 | Plus | 128187-128383 |
| 405194 | 7230072 | Plus | 190465-190645, 193346-193610 |
| 405245 | 7249293 | Minus | 57560-58312 |
| 405365 | 2275192 | Minus | 119867-120372, 120481-120824, 121029-121357 |
| 405376 | 1552533 | Plus | 28875-29099 |
| 405418 | 6997292 | Plus | 51839-51953 |
| 405436 | 7408068 | Minus | 55716-55979 |
| 405443 | 7408143 | Plus | 90716-90887, 101420-101577 |
| 405474 | 8439781 | Plus | 172005-172175 |
| 405502 | 9211311 | Minus | 50360-50584 |
| 405580 | 4512267 | Plus | 169232-169647 |
| 405595 | 7159256 | Plus | 47585-47688 |
| 405630 | 4508116 | Minus | 103218-103291, 105858-105993, 110051-110126 |
| 405646 | 4914350 | Plus | 741-969 |
| 405710 | 5531256 | Minus | 66203-66832 |
| 405806 | 7274891 | Minus | 224961-226780 |
| 405812 | 4775630 | Minus | 29424-29764 |
| 405855 | 7652031 | Minus | 60377-60795 |
| 405879 | 6758747 | Minus | 54789-55457 |
| 405891 | 6758795 | Plus | 41062-41861 |
| 405902 | 6758795 | Minus | 82322-83110 |
| 405932 | 7767812 | Minus | 123525-123713 |
| 406038 | 8389537 | Plus | 37764-37877 |
| 406140 | 9168231 | Minus | 49887-50219 |
| 406181 | 5923650 | Plus | 16586-16855 |
| 406231 | 7417725 | Plus | 17206-17641, 17772-17968 |
| 406248 | 7417725 | Plus | 49711-50227 |
| 406274 | 7543787 | Plus | 932-1123 |
| 406356 | 7107907 | Plus | 18761-18973 |
| 406404 | 9256305 | Minus | 152569-152874 |
| 406467 | 9795551 | Plus | 182212-182958 |
| 406557 | 7711569 | Minus | 5446-5574, 6170-6352 |
| 406575 | 7711679 | Plus | 142034-142473 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 10A
Genes preferentially expressed in non-invasive bladder tumors

| Pkey | ExAccn | UnigeneID | Unigene Title | R1 |
|---|---|---|---|---|
| 421110 | AJ250717 | Hs.1355 | cathepsin E | 8.23 |
| 428651 | AF196478 | Hs.188401 | annexin A10 | 5.78 |
| 451668 | Z43948 | Hs.326444 | cartilage acidic protein 1 | 5.53 |
| 415511 | AI732617 | Hs.182362 | ESTs | 4.72 |
| 428336 | AA503115 | Hs.183752 | microseminoprotein, beta- | 4.66 |
| 418026 | BE379727 | Hs.83213 | fatty acid binding protein 4, adipocyte | 4.62 |
| 400752 | | | NM_003105*: Homo sapiens sortilin-related | 3.99 |
| 430315 | NM_004293 | Hs.239147 | guanine deaminase | 3.82 |
| 403010 | | | C21000152: gi\|6226483\|sp\|Q52118\|YMO3_ERWS | 3.56 |
| 404977 | | | Insulin-like growth factor 2 (somatomedi | 3.54 |
| 426657 | NM_015865 | Hs.171731 | solute carrier family 14 (area transport | 3.51 |
| 400409 | AF153341 | | Homo sapiens winged helix/forkhead trans | 3.38 |
| 400844 | | | NM_003105*: Homo sapiens sortilin-related | 3.27 |
| 406081 | | | Target Exon | 3.22 |
| 417275 | X63578 | Hs.295449 | parvalbumin | 3.03 |
| 402230 | | | Target Exon | 2.96 |
| 454219 | X75042 | Hs.44313 | v-rel avian reticuloendotheliosis viral | 2.89 |
| 403381 | | | ENSP00000231844*: Ecotropic virus integra | 2.87 |
| 426088 | AF038007 | Hs.166196 | ATPase, Class I, type 8B, member 1 | 2.86 |
| 452286 | AI358570 | Hs.123933 | ESTs, Weakly similar to ZN91_HUMAN ZINC | 2.69 |
| 434061 | AW024973 | Hs.283675 | NPD009 protein | 2.66 |
| 418406 | X73501 | Hs.84905 | cytokeratin 20 | 2.65 |
| 418818 | AA228899 | Hs.101307 | Homo sapiens HUT11 protein mRNA, partial | 2.59 |
| 421594 | R45689 | Hs.21889 | Homo sapiens cDNA FLJ12978 fis, clone NT | 2.57 |
| 403383 | | | Target Exon | 2.56 |
| 435563 | AF210317 | Hs.95497 | solute carrier family 2 (facilitated glu | 2.55 |
| 424800 | AL035588 | Hs.153203 | MyoD family inhibitor | 2.54 |
| 404606 | | | Target Exon | 2.53 |
| 418205 | L21715 | Hs.83760 | troponin I, skeletal, fast | 2.53 |
| 431912 | AI660552 | Hs.76549 | ESTs, Weakly similar to A56154 Abl subst | 2.52 |
| 413786 | AW613780 | Hs.13500 | ESTs | 2.51 |
| 421100 | AW351839 | Hs.124660 | Homo sapiens cDNA: FLJ21763 fis, clone C | 2.50 |
| 416640 | BE262478 | Hs.79404 | neuron-specific protein | 2.50 |
| 420729 | AW964897 | Hs.290825 | ESTs | 2.50 |
| 402844 | | | C1000118*: gi\|9951913\|ref\|NP_062832.1\| pr | 2.48 |
| 401093 | | | C12000586*: gi\|6330167\|db\|BAA86477.1\| (A | 2.46 |
| 417720 | AA205625 | Hs.208067 | ESTs | 2.45 |
| 400297 | AI127076 | Hs.306201 | hypothetical protein DKFZp564O1278 | 2.45 |
| 403818 | | | Target Exon | 2.44 |
| 440273 | AI805392 | Hs.325335 | Homo sapiens cDNA: FLJ23523 fis, clone L | 2.44 |
| 418060 | AA211589 | Hs.208047 | ESTs | 2.40 |
| 400843 | | | NM_003105*: Homo sapiens sortilin-related | 2.38 |
| 446006 | NM_004403 | Hs.13530 | deafness, autosomal dominant 5 | 2.35 |
| 401512 | | | NM_014080: Homo sapiens dual oxidase-like | 2.34 |
| 446847 | T51454 | Hs.82845 | Homo sapiens cDNA: FLJ21930 fis, clone H | 2.32 |
| 417094 | NM_006895 | Hs.81182 | histamine N-methyltransferase | 2.31 |
| 436293 | AI601188 | Hs.120910 | ESTs | 2.30 |
| 436246 | AW450963 | Hs.119991 | ESTs | 2.30 |
| 447578 | AA912347 | Hs.136585 | ESTs, Weakly similar to JC5314 CDC28/cdc | 2.29 |
| 417381 | AF164142 | Hs.82042 | solute carrier family 23 (nucleobase tra | 2.28 |
| 426028 | NM_001110 | Hs.172028 | a disintegrin and metalloproteinase doma | 2.27 |
| 431448 | AL137517 | Hs.306201 | hypothetical protein DKFZp564O1278 | 2.26 |
| 437181 | AI306615 | Hs.125343 | ESTs, Weakly similar to KIAA0758 protein | 2.23 |
| 415025 | AW207091 | Hs.72307 | ESTs | 2.18 |
| 412610 | X90908 | Hs.74126 | fatty acid binding protein 6, ileal (gas | 2.04 |
| 424099 | AF071202 | Hs.139336 | ATP-binding cassette, sub-family C (CFTR | 2.03 |
| 433078 | AW015188 | Hs.121575 | Homo sapiens cDNA FLJ12231 fis, clone MA | 2.01 |
| 416225 | AA577730 | Hs.188684 | ESTs, Weakly similar to PC4259 ferritin | 2.00 |
| 411880 | AW872477 | | gb: hm30f03.x1 NCI_CGAP_Thy4 Homo sapiens | 1.99 |
| 452316 | AA298484 | Hs.61265 | ESTs, Moderately similar to G786_HUMAN P | 1.89 |
| 413804 | T64682 | | gb: yc48b02.r1 Stratagene liver (937224) | 1.88 |
| 432306 | Y18207 | Hs.303090 | protein phosphatase 1, regulatory (inhib | 1.76 |
| 405364 | | | ENSP00000239138*: Guanine nucleotide-bind | 1.60 |
| 414320 | U13616 | Hs.75893 | ankyrin 3, node of Ranvier (ankyrin G) | 1.52 |
| 401929 | | | C17001690:gi\|6005701\|ref\|NP_009099.1\| AT | 1.00 |

Pkey: Unique Eos probeset identifier number
ExAccn: Exemplar Accession number, Genbank accession number
UnigeneID: Unigene number
Unigene Title: Unigene gene title
R1 80th percentile of Ta tumor Als divided by the 80th percentile of T2-T4 tumor Als

TABLE 10B

| Pkey | CAT Number | Accession |
|---|---|---|
| 411880 | 1263110_1 | AW872477 BE088101 T05990 |
| 413804 | 1390710_1 | T64682 BE168190 BE168256 |

Pkey: Unique Eos probeset identifier number
CAT number: Gene cluster number
Accession: Genbank accession numbers

TABLE 10C

| Pkey | Ref | Strand | Nt_position |
|---|---|---|---|
| 400752 | 7331445 | Minus | 36215-36461 |
| 400843 | 9188605 | Plus | 5863-5970, 7653-7784, 8892-9023, |
| | | | 9673-9807, 10634-10789, |
| | | | 15254-15403, 23827-23958 |
| 400844 | 9188605 | Plus | 24746-24872, 25035-25204 |
| 401093 | 8516137 | Minus | 22335-23166 |
| 401512 | 7622346 | Plus | 136399-136557 |
| 401929 | 3810670 | Minus | 3167-3286, 4216-4310 |
| 402230 | 9966312 | Minus | 29782-29932 |
| 402844 | 9369286 | Plus | 54958-55313 |
| 403010 | 3132346 | Plus | 78385-79052 |
| 403381 | 9438267 | Minus | 26009-26178 |
| 403383 | 9438267 | Minus | 119837-121197 |
| 403818 | 8962065 | Minus | 138360-138512, 144656-144796 |
| 404606 | 9212936 | Minus | 22310-23269 |
| 404977 | 3738341 | Minus | 43081-43229 |
| 405364 | 2281075 | Minus | 48325-48491, 49136-49252 |
| 406081 | 9123861 | Minus | 38115-38691 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 11A
Genes preferentially expressed in muscle-invasive bladder tumors

| Pkey | ExAccn | UnigeneID | Unigene Title | R1 |
|---|---|---|---|---|
| 423961 | D13666 | Hs.136348 | periostin (OSF-2os) | 11.22 |
| 421948 | L42583 | Hs.334309 | keratin 6A | 10.87 |
| 401780 | | | NM_005557*: Homo sapiens keratin 16 (foca | 9.16 |
| 446619 | AU076643 | Hs.313 | secreted phosphoprotein 1 (osteopontin, | 8.40 |
| 444381 | BE387335 | Hs.283713 | ESTs, Weakly similar to S64054 hypotheti | 7.88 |
| 439926 | AW014875 | Hs.137007 | ESTs | 7.73 |
| 408243 | Y00787 | Hs.624 | interleukin 8 | 7.54 |
| 414183 | AW957446 | Hs.301711 | ESTs | 7.00 |
| 411573 | AB029000 | Hs.70823 | KIAA1077 protein | 6.52 |
| 414522 | AW518944 | Hs.76325 | step II splicing factor SLU7 | 6.42 |
| 413063 | AL035737 | Hs.75184 | chitinase 3-like 1 (cartilage glycoprote | 6.14 |
| 441633 | AW958544 | Hs.112242 | normal mucosa of esophagus specific 1 | 6.04 |
| 427337 | Z46223 | Hs.176663 | Fc fragment of lgG, low affinity IIIb, r | 5.66 |
| 420859 | AW468397 | Hs.100000 | S100 calcium-binding protein A8 (calgran | 5.62 |
| 422168 | AA586894 | Hs.112408 | S100 calcium-binding protein A7 (psorias | 5.51 |
| 418870 | AF147204 | Hs.89414 | chemokine (C-X-C motif), receptor 4 (fus | 5.48 |
| 401781 | | | Target Exon | 5.46 |
| 421116 | T19132 | Hs.101850 | retinol-binding protein 1, cellular | 5.41 |
| 448429 | D17408 | Hs.21223 | calponin 1, basic, smooth muscle | 5.41 |
| 414020 | NM_002984 | Hs.75703 | small inducible cytokine A4 (homologous | 5.32 |
| 447526 | AL048753 | Hs.303649 | small inducible cytokine A2 (monocyte ch | 5.32 |
| 424247 | X14008 | Hs.234734 | lysozyme (renal amyloidosis) | 5.27 |
| 456525 | AW468397 | Hs.100000 | S100 calcium-binding protein A8 (calgran | 5.22 |
| 418007 | M13509 | Hs.83169 | matrix metalloproteinase 1 (interstitial | 5.17 |
| 406663 | U24683 | Hs.293441 | immunoglobulin heavy constant mu | 5.08 |
| 425593 | AA278921 | Hs.1908 | proteoglycan 1, secretory granule | 4.93 |
| 433336 | AF017986 | Hs.31386 | secreted frizzled-related protein 2 | 4.89 |
| 425118 | AU076611 | Hs.154672 | methylene tetrahydrofolate dehydrogenase | 4.74 |
| 415994 | NM_002923 | Hs.78944 | regulator of G-protein signalling 2, 24 k | 4.64 |
| 412326 | R07566 | Hs.73817 | small inducible cytokine A3 (homologous | 4.39 |
| 422158 | L10343 | Hs.112341 | protease inhibitor 3, skin-derived (SKAL | 4.30 |
| 446921 | AB012113 | Hs.16530 | small inducible cytokine subfamily A (Cy | 4.29 |
| 433470 | AW960564 | | transmembrane 4 superfamily member 1 | 4.23 |
| 417880 | BE241595 | Hs.82848 | selectin L (lymphocyte adhesion molecule | 4.22 |
| 446500 | U78093 | Hs.15154 | sushi-repeat-containing protein, X chrom | 4.22 |
| 413324 | V00571 | Hs.75294 | corticotropin releasing hormone | 4.20 |
| 436729 | BE621807 | | transmembrane 4 superfamily member 1 | 4.18 |
| 450455 | AL117424 | Hs.25035 | chloride intracellular channel 4 | 4.15 |
| 413731 | BE243845 | Hs.75511 | connective tissue growth factor | 4.09 |
| 412429 | AV650262 | Hs.75765 | GRO2 oncogene | 4.00 |
| 418283 | S79895 | Hs.83942 | cathepsin K (pycnodysostosis) | 4.00 |
| 418299 | AA279530 | Hs.83968 | integrin, beta 2 (antigen CD18 (p95), ly | 4.00 |
| 420899 | NM_001629 | Hs.100194 | arachidonate 5-lipoxygenase-activating p | 3.97 |
| 400288 | X06256 | Hs.149609 | integrin, alpha 5 (fibronectin receptor, | 3.95 |
| 437446 | AA788946 | Hs.101302 | ESTs, Moderately similar to CA1C RAT COL | 3.94 |
| 413441 | AI929374 | Hs.75367 | Src-like-adapter | 3.91 |
| 404854 | | | Target Exon | 3.81 |
| 431319 | AA873350 | Hs.302232 | ESTs | 3.77 |
| 452432 | AW206008 | Hs.283378 | Homo sapiens cDNA: FLJ21778 fis, clone H | 3.76 |
| 429679 | NM_006290 | Hs.211600 | tumor necrosis factor, alpha-induced pro | 3.72 |
| 428330 | L22524 | Hs.2256 | matrix metalloproteinase 7 (matrilysin, | 3.58 |
| 408380 | AF123050 | Hs.44532 | diubiquitin | 3.58 |
| 431103 | M57399 | Hs.44 | pleiotrophin (heparin binding growth fac | 3.57 |
| 422545 | X02761 | Hs.287820 | fibronectin 1 | 3.52 |
| 418203 | X54942 | Hs.83758 | CDC28 protein kinase 2 | 3.49 |
| 409956 | AW103364 | Hs.727 | inhibin, beta A (activin A, activin AB a | 3.48 |
| 406687 | M31126 | | matrix metalloproteinase 11 (stromelysin | 3.41 |
| 414359 | M62194 | Hs.75929 | cadherin 11, type 2, OB-cadherin (osteob | 3.36 |
| 417259 | AW903838 | Hs.81800 | chondroitin sulfate proteoglycan 2 (vers | 3.32 |
| 417497 | AW402482 | Hs.82212 | CD53 antigen | 3.30 |
| 449335 | AW150717 | Hs.345728 | STAT induced STAT inhibitor 3 | 3.25 |
| 445033 | AV652402 | Hs.72901 | mucin 13, epithelial transmembrane | 3.23 |
| 427274 | NM_005211 | Hs.174142 | colony stimulating factor 1 receptor, fo | 3.23 |
| 427527 | AI809057 | Hs.293441 | immunoglobulin heavy constant mu | 3.22 |
| 409142 | AL136877 | Hs.50758 | SMC4 (structural maintenance of chromoso | 3.18 |
| 453331 | AI240665 | | ESTs | 3.15 |
| 428036 | AW068302 | | Homo sapiens mRNA for caldesmon, 3′ UTR | 3.10 |
| 417366 | BE185289 | Hs.1076 | small proline-rich protein 1B (cornifin) | 3.09 |
| 414622 | AI752666 | Hs.76669 | nicotinamide N-methyltransferase | 3.07 |
| 418478 | U38945 | Hs.1174 | cyclin-dependent kinase inhibitor 2A (me | 3.02 |
| 417771 | AA804698 | Hs.82547 | retinoic acid receptor responder (tazaro | 2.77 |
| 413936 | AF113676 | Hs.297681 | serine (or cysteine) proteinase inhibito | 2.75 |
| 406755 | N80129 | Hs.94360 | metallothionein 1L | 2.75 |
| 426653 | AA530892 | Hs.171695 | deal specificity phosphatase 1 | 2.67 |
| 443623 | AA345519 | Hs.9641 | complement component 1, q subcomponent, | 2.65 |
| 443907 | AU076484 | Hs.9963 | TYRO protein tyrosine kinase binding pro | 2.64 |
| 422048 | NM_012445 | Hs.288126 | spondin 2, extracellular matrix protein | 2.51 |
| 410204 | AJ243425 | Hs.326035 | early growth response 1 | 2.46 |
| 438973 | AW959503 | Hs.60440 | ESTs, Weakly similar to serin protease w | 2.46 |
| 420202 | AL036557 | Hs.95910 | putative lymphocyte G0/G1 switch gene | 2.44 |
| 422626 | AA344932 | Hs.118786 | metallothionein 2A | 2.44 |
| 442402 | NM_000954 | Hs.8272 | prostaglandin D2 synthase (21 kD, brain) | 2.43 |
| 413902 | AU076743 | Hs.75613 | CD36 antigen (collagen type I receptor, | 2.42 |
| 434868 | R50032 | Hs.159263 | collagen, type VI, alpha 2 | 2.42 |
| 407207 | T03651 | Hs.336780 | tubulin, beta polypeptide | 2.30 |
| 438855 | AW946276 | Hs.6441 | Homo sapiens mRNA; cDNA DKFZp586J021 (fr | 2.29 |
| 430413 | AW842182 | Hs.241392 | small inducible cytokine A5 (RANTES) | 2.20 |
| 424909 | S78187 | Hs.153752 | cell division cycle 25B | 2.18 |
| 419938 | AU076772 | Hs.1279 | complement component 1, r subcomponent | 2.17 |
| 416819 | U77735 | Hs.80205 | pim-2 oncogene | 2.11 |
| 422562 | AI962060 | Hs.118397 | AE-binding protein 1 | 2.07 |
| 414081 | AW969976 | Hs.279009 | matrix Gla protein | 2.07 |
| 426406 | AI742501 | Hs.169756 | complement component 1, s subcomponent | 2.03 |
| 443950 | NM_001425 | Hs.9999 | epithelial membrane protein 3 | 2.01 |
| 418323 | NM_002118 | Hs.1162 | major histocompatibility complex, class | 1.94 |
| 414420 | AA043424 | Hs.76095 | immediate early response 3 | 1.90 |
| 415149 | X12451 | Hs.78056 | cathepsin L | 1.72 |
| 415213 | NM_002933 | Hs.78224 | ribonuclease, RNase A family, 1 (pancrea | 1.70 |
| 421848 | X15880 | Hs.108885 | collagen, type VI, alpha 1 | 1.69 |
| 452516 | AA058630 | Hs.29759 | RNA POLYMERASE I AND TRANSCRIPT RELEASE | 1.64 |
| 427676 | AA394062 | Hs.300772 | tropomyosin 2 (beta) | 1.64 |
| 415198 | AW009480 | Hs.943 | natural killer cell transcript 4 | 1.60 |
| 424390 | AW815657 | Hs.182241 | interferon induced transmembrane protein | 1.59 |
| 426825 | AL133415 | Hs.297753 | vimentin | 1.51 |
| 452363 | AI582743 | Hs.94953 | Homo sapiens, Similar to complement comp | 1.46 |
| 407694 | U77594 | Hs.37682 | retinoic acid receptor responder (tazaro | 1.44 |

Pkey: Unique Eos probeset identifier number
ExAccn: Exemplar Accession number, Genbank accession number
UnigeneID: Unigene number
Unigene Title: Unigene gene title
R1 80th percentile of T2-T4 tumor Als divided by the 80th percentile of Ta tumor Als

TABLE 11B

| Pkey | CAT Number | Accession |
|---|---|---|
| 428036 | 28620_1 | AW068302 AI754558 AI750727 AI752631 AA302174 AA327522 M64110 AW859944 AW859989 AI751995 |
| | | AA769620 AI858829 AI924875 AI888836 AA864291 AI685060 AW088029 AI924908 AW466328 |
| | | AI093800 AA991651 AI254601 BE004703 AA334442 AW938852 AA194330 |
| | | AL046953 AA852866 AW391995 W30846 AW662928 W25261 AA042863 R99045 H97060 |
| | | W03910 H94687 T88984 AL048165 T29632 N31556 N36484 AI798679 AA989355 W23832 |
| | | AA873789 AI743646 AA363587 AI814748 AW338990 N73740 N83666 AL047816 R24137 |
| | | R63433 AA524984 AA234043 AA195131 N99903 AA453669 AI240302 AA370271 AI950026 |
| | | AW771049 AA121476 AA569557 AI752632 AI355594 AI471993 AI159941 N94555 AI753138 |
| | | N21537 H97881 N25769 AW068044 AA808425 R63380 AA384736 AA384738 AA852352 |
| | | AI073645 AA527960 AA525036 AA044414 AI752460 AA703064 R01216 AA897183 AI751996 |
| | | T81078 H95047 AA573642 D58348 N20953 AA437143 N95439 AA579540 AW867056 |
| | | AA770090 AI085180 AI806799 AA426421 AI572513 R24081 AA853189 AA295620 AA234044 |
| | | AA371020 AW994984 H20896 AW964438 AA318516 AA318499 AA318727 AA318211 |
| | | AA318478 AA318444 AA318307 AA318497 AA318448 AA318309 AA318496 AA318213 |
| | | AA318435 AA318424 AA318217 AA318523 AA318438 AA318487 AA318724 AA593185 |
| | | AW994985 T69842 AI251813 AA478174 AA447737 T68350 F07712 AA121145 H08973 |
| | | AA345212 BE000667 AW068210 AW608407 R05674 H16712 N85426 N42354 H85516 |
| | | BE147991 T28113 R32662 AA384678 AW239275 H82382 AW840700 D58229 C04082 |
| | | W45394 AW795667 R73973 BE002409 AA042828 AA363555 AJ223812 AA344709 |
| | | BE149590 R70995 W46881 W90778 N71242 AA534826 AL040676 R23797 H96450 |
| | | AA062957 D79947 W46960 AW959278 AA295997 AA026215 AW579469 AW365135 |
| | | AW365134 AW994353 AW972886 AW069166 AA343690 AW888731 AI751527 AA937490 |
| | | AA937506 AI826715 BE465604 AI925532 AI858109 AW339097 AI858524 AI720571 |
| | | BE04650 AW384981 AA043908 AA375983 AA525181 AW068366 AW070577 AW891837 |
| | | N83985 AW182753 AI422979 AI679733 BE006555 AL048166 AI081401 AI888821 AI626043 |
| | | N37087 AI624140 AI801298 AA600048 AI753947 H89615 N66424 AW069756 AI814880 |
| | | AI982806 AI754287 AI971816 AW069022 AW069069 AW069454 AA342989 AI077712 |
| | | AI311467 AI087361 AI801015 W46993 AI281324 AW191963 AI421675 AI300881 AI356670 |
| | | AA873156 AI004219 AI189685 AA478018 AA076063 AI445222 AI753124 AI521569 |
| | | AI925026 AI022368 AI475993 H20846 AI223234 AI635123 AA579170 N30442 AW117889 |
| | | AA807935 AA558975 AI306636 AA888963 AI952591 AI935835 AI445293 H16713 |
| | | AW139833 AA622122 AA972051 AI280828 H09543 AI453725 AW069613 AI865615 |
| | | AI753921 AI368782 AI633208 AI446651 W46961 N22201 H82276 C16555 AA291477 |
| | | AW440535 AW517755 AA669921 AI926777 AW662118 AA553369 N67873 AW023948 |
| | | C15861 C16601 AI251465 AW079187 BE045090 AI273006 C16390 C16503 AI620823 |
| | | F13661 N66864 Z21311 C16108 C16089 C16400 AA758273 AI287781 AA864676 |
| | | AW608074 AW385583 AI589944 AA665817 AW192979 AW469065 M564048 H84715 |
| | | C16417 AA731072 AA661674 C16487 N29477 AW189997 AI370492 C16471 AA652809 |
| | | AA936687 AA506512 C16306 AW028413 AI537935 AA528347 C16255 AW029046 C16202 |
| | | AI868152 AI524662 T94414 AI567041 AI619654 AW008486 AI075624 AA577434 AA345104 |
| | | T30105 AA932002 C16585 AI750390 AW294265 AI619552 AA669781 AA026678 AW132002 |
| | | AW263919 C16562 AA759137 AA693351 Z40779 C16577 AA885045 AW073763 R45484 |
| | | AI520895 U54708 T49285 AI568126 AW006569 AI093317 AL119781 T61046 AI053563 |
| | | H51958 AF114144 AA305739 AW950394 AW793928 AW793910 AL047737 AV659047 |
| | | AV659632 AI750389 AA092053 AA092798 H85367 T61597 R23745 Z20418 T78485 |
| | | AI751528 AW068121 AA853188 AI752459 AA853711 AW950663 R78964 R36359 |
| | | R21626 R21522 |
| 433470 | 3672_1 | AW960564 AA092457 T55890 D56120 T92525 AI815987 |
| | | BE182608 BE182595 AW080238 M90657 AA347236 AW961686 AW176446 |
| | | AA304671 AW583735 T61714 AA316968 AI446615 AA343532 AA083489 AA488005 |
| | | W52095 W39480 N57402 D82638 W25540 W52847 D82729 D58990 BE619182 |
| | | AA315188 AA308636 AA112474 W76162 AA088544 H52265 AA301631 H80982 |
| | | AA113786 BE620997 AW651691 AA343799 BE613669 BE547180 BE546656 F11933 |
| | | AA376800 AW239185 AA376086 BE544387 BE619041 AA452515 AA001806 AA190873 |
| | | AA180483 AA159546 F00242 AI940609 AI940602 AI189753 T97663 T66110 AW062896 |
| | | AW062910 AW062902 AI051622 AI828930 AA102452 AI685095 AI819390 AA557597 |
| | | AA383220 AI804422 AI633575 AW338147 AW603423 AW606800 AW750567 |
| | | AW510672 AI250777 AA083510 AW629109 AW513200 AA921353 AI677934 |
| | | AI148698 AI955858 AA173825 AA453027 AI027865 AW375542 AA454099 AA733014 |
| | | AI591384 R79300 R80023 AA843108 AA626058 AA844898 AW375550 AA889018 |
| | | AI474275 AW205937 AI052270 AW388117 AW388111 AA699452 AI242230 N47476 |
| | | H38178 AA366621 AA113196 AA130023 H39740 T61629 AI885973 AW083671 |
| | | AA179730 AA305757 AI285455 N83956 AA216013 AA336155 AW999959 T97525 |
| | | AA345349 T91762 AA771981 AI285092 AI591386 BE392486 BE385852 AA682601 |
| | | AI682884 AA345840 T85477 AA292949 AA932079 AA098791 D82607 T48574 |
| | | AW752038 C06300 |
| 436729 | 42585_1 | BE621807 AI445461 AI346835 AI453743 AI564644 |
| | | AI928364 AW984527 BE156214 AI694111 AI591358 C17504 C17476 C17963 C18304 |
| | | AW071625 AI678712 C17732 D57559 H61762 AI720939 AI262930 H27252 AA479712 |
| | | AI927769 AA291465 AA155661 AI963432 AI567995 AA421678 AI925607 AA292956 |
| | | AA192448 AW192593 AI865838 AI696905 AI871950 AI911921 BE619741 BE439796 |
| | | AI161312 AI597801 AI424384 AI093510 AI240988 AW820230 AI492554 BE044033 |
| | | AW262737 AW008570 AA043216 AW629505 AA136645 AA037722 AA706057 |
| | | AA088439 AW806193 AW806183 AA479834 BE501957 AA129574 R38114 AA649494 |
| | | AA524526 BE327120 AW572531 BE219784 BE349186 AW015724 AA043217 |
| | | AW772000 AI799814 AI671727 AW779725 AA502832 AI470033 AA129575 W38161 |
| | | AI972739 AA404570 AA627686 AA723200 AA147228 AA903050 AI990245 AI075878 |
| | | T32487 C06123 AA157944 AI800106 W60075 AI859160 AA478328 AW673152 AA182640 |
| | | AI990827 AW275048 AW103470 AI298935 AW471421 R79190 AW085158 W45410 |
| | | AI333170 AW300456 AA662517 T55840 AI823466 AI692846 AA962397 AW191997 |
| | | AA136658 AI251817 BE044134 AW339104 AW517762 AA724739 R79933 AA411100 |
| | | AA191349 AA037696 AA190966 AA757735 AW772283 AA010631 H80983 AI769516 |
| | | H64985 AI061065 AI950693 AA085492 AI245632 H28594 AW088968 BE156360 AI349390 |
| | | AI621320 AI738844 AW194272 AA148284 AA953883 C06365 AA487893 AI927217 |
| | | AI918523 AI453453 AI798502 AI189366 AI261359 AI032569 AW338678 AI972899 AI500576 |
| | | AI872628 AI693030 Z28771 AI985583 AI363829 AW339301 AA581093 AI650338 W60032 |
| | | AA603586 AI686240 AW242958 AA719173 AI745717 AW675302 AI582462 AI244845 |
| | | AI565439 F09579 AI918453 AA035576 AI472527 AW351556 AA191414 AW674145 |
| | | D57558 AI446740 D57845 AI589264 C05782 AA722206 AI432033 R21752 |
| | | BE157510 AI829640 AI468237 AW384233 AA989662 AI865912 AW197954 AI344941 |
| | | X75684 AI344943 AW583310 AA988297 AI334860 AI348877 AI798415 D11921 AI377596 |
| | | AI983655 AI744233 C06111 AI248307 AA948565 AI224807 |
| 453331 | 96214_1 | AI240665 T53681 N77468 H51833 AA147247 R75732 C18450 |
| | | R73999 AI095755 T49904 H03868 AA411580 R33395 AA410586 T48869 |
| | | D63292 R31981 H12498 H02668 AA035018 R75957 AI803329 R27528 R36203 AI809932 |
| | | AI808765 R78948 AA411449 AA976929 AI378760 AI378620 T48870 R73906 R75632 |
| | | H03612 AA909684 N50695 H02580 H12839 N58781 AA742532 AI360919 H03502 |
| | | BE208298 R68588 AI350463 R31935 AW069127 AA411621 R25671 R36105 H12451 |
| | | H03869 H51263 AA035486 R25109 R25110 AA147933 |

Pkey: Unique Eos probeset identifier number
CAT number: Gene cluster number
Accession: Genbank accession numbers

TABLE 11C

| Pkey | Ref | Strand | Nt_position |
|---|---|---|---|
| 401780 | 7249190 | Minus | 28397-28617, 28920-29045, |
| | | | 29135-29296, 29411-29567, |
| | | | 29705-29787 30224-30573 |
| 401781 | 7249190 | Minus | 83215-83435, 83531-83656, |
| | | | 83740-83901, 84237-84393, |
| | | | 84955-85037, |
| | | | 86290-86814 |
| 404854 | 7143420 | Plus | 14260-14537 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.* Dunham I. et al., Nature (1999) 402: 489-495.
Strand: Indicates DNA strand from which exons were predicted.
Nt_position: Indicates nucleotide positions of predicted exons.

TABLE 12A
Genes preferentially expressed in muscle-invasive bladder tumors

| Pkey | ExAccn | UnigeneID | Unigene Title | Seq ID No. |
|---|---|---|---|---|
| 424503 | NM_002205 | Hs.149609 | integrin, alpha 5 (fibronectin receptor, | Seq ID No. 1 & 2 |
| 400289 | X07820 | Hs.2258 | matrix metalloproteinase 10 (stromelysin | Seq ID No. 3 & 4 |
| 418007 | M13509 | Hs.83169 | matrix metalloproteinase 1 (interstitial | Seq ID No. 5 & 6 |
| 418738 | AW388633 | Hs.6682 | solute carrier family 7, (cationic amino | Seq ID No. 7 & 8 |
| 406964 | M21305 | | FGENES predicted novel secreted protein | Seq ID No. 9 & 10 |
| 429276 | AF056085 | Hs.198612 | G protein-coupled receptor 51 | Seq ID No. 11 & 12 |
| 428227 | AA321649 | Hs.2248 | small inducible cytokine subfamily B (Cy | Seq ID No. 13 & 14 |
| 410102 | AW248508 | Hs.279727 | ESTs; homologue of PEM-3 [Ciona savignyi | Seq ID No. 15 & 16 |
| 425247 | NM_005940 | Hs.155324 | matrix metalloproteinase 11 (stromelysin | Seq ID No. 17 & 18 |
| 424735 | U31875 | Hs.272499 | short-chain alcohol dehydrogenase family | Seq ID No. 19 & 20 |
| 420159 | AI572490 | Hs.99785 | Homo sapiens cDNA: FLJ21245 fis, clone C | Seq ID No. 21 & 22 |
| 415511 | AI732617 | Hs.182362 | ESTs | Seq ID No. 23-25 |
| 452461 | N78223 | Hs.108106 | transcription factor | Seq ID No. 26 & 27 |
| 413324 | V00571 | Hs.75294 | corticotropin releasing hormone | Seq ID No. 28 & 29 |
| 443211 | AI128388 | Hs.143655 | ESTs | Seq ID No. 30 |
| 439926 | AW014875 | Hs.137007 | ESTs | Seq ID No. 31 & 32 |
| 432222 | AI204995 | | gb: an03c03.x1 Stratagene schizo brain S1 | Seq ID No. 33 |
| 408908 | BE296227 | Hs.250822 | serine/threonine kinase 15 | Seq ID No. 34 & 35 |
| 443171 | BE281128 | Hs.9030 | TONDU | Seq ID No. 36 & 37 |
| 432829 | W60377 | Hs.57772 | ESTs | Seq ID No. 38 & 39 |
| 410553 | AW016824 | Hs.272068 | hypothetical protein MGC14128 | Seq ID No. 40 & 41 |
| 419183 | U60669 | Hs.89663 | cytochrome P450, subfamily XXIV (vitamin | Seq ID No. 42 & 43 |
| 425721 | AC002115 | Hs.159309 | uroplakin 1A | Seq ID No. 44 & 45 |
| 420370 | Y13645 | Hs.97234 | uroplakin 2 | Seq ID No. 46 & 47 |
| 437852 | BE001836 | Hs.256897 | ESTs, Weakly similar to dJ365O12.1 [H. sa | Seq ID No. 48 & 49 |
| 428664 | AK001666 | Hs.189095 | similar to SALL1 (sal (Drosophila)-like | Seq ID No. 50 & 51 |
| 456034 | AW450979 | | gb: UI-H-BI3-ala-a-12-0-UI.s1 NCL_CGAP_Su | Seq ID No. 52 |
| 421110 | AJ250717 | Hs.1355 | cathepsin E | Seq ID No. 53 & 54 |
| 451668 | Z43948 | Hs.326444 | cartilage acidic protein 1 | Seq ID No. 55-60 |
| 408243 | Y00787 | Hs.624 | interleukin 8 | Seq ID No. 61 & 62 |
| 440304 | BE159984 | Hs.125395 | ESTs | Seq ID No. 63 & 64 |
| 414918 | AI219207 | Hs.72222 | hypothetical protein FLJ13459 | Seq ID No. 65 & 66 |
| 418867 | D31771 | Hs.89404 | msh (Drosophila) homeo box homolog 2 | Seq ID No. 67 & 68 |
| 426088 | AF038007 | Hs.166196 | ATPase, Class I, type 8B, member 1 | Seq ID No. 69 & 70 |
| 405033 | | | C1002652*: gi\|544327\|sp\|Q04799\|FMO5_RABIT | Seq ID No. 71 & 72 |
| 422282 | AF019225 | Hs.114309 | apolipoprotein L | Seq ID No. 73 & 74 |
| 425852 | AK001504 | Hs.159651 | death receptor 6, TNF superfamily member | Seq ID No. 75 & 76 |
| 400844 | | | NM_003105*: Homo sapiens sortilin-related | Seq ID No. 77 & 78 |
| 404875 | | | NM_022819*: Homo sapiens phospholipase A2 | Seq ID No. 79 & 80 |
| 422809 | AK001379 | Hs.121028 | hypothetical protein FLJ10549 | Seq ID No. 81 & 82 |
| 431347 | AI133461 | Hs.251664 | insulin-like growth factor 2 (somatomedi | Seq ID No. 83 & 84 |
| 413804 | T64682 | | gb: yc48b02.r1 Stratagene liver (937224) | Seq ID No. 85 & 86 |
| 444163 | AI126098 | | FGENESH predicted RNaseH domain-containi | Seq ID No. 87-89 |
| 444444 | AI149332 | Hs.14855 | ESTs | Seq ID No. 90 & 91 |
| 427747 | AW411425 | Hs.180655 | serine/threonine kinase 12 | Seq ID No. 92 & 93 |
| 419741 | NM_007019 | Hs.93002 | ubiquitin carrier protein E2-C | Seq ID No. 94 & 95 |
| 420281 | AI623693 | Hs.323494 | Predicted cation efflux pump | Seq ID No. 96 & 97 |
| 402305 | | | C19000735*: gi\|4508027\|ref\|NP_003414.1\|z | Seq ID No. 98-100 |
| 436608 | AA628980 | | down syndrome critical region protein DS | Seq ID No. 101 & 102 |
| 427239 | BE270447 | | ubiquitin carrier protein | Seq ID No. 103 & 104 |
| 413278 | BE563085 | Hs.833 | interferon-stimulated protein, 15 kDa | Seq ID No. 105 & 106 |
| 424008 | R02740 | Hs.137555 | putative chemokine receptor; GTP-binding | Seq ID No. 107 & 108 |
| 414907 | X90725 | Hs.77597 | polo (Drosophia)-like kinase | Seq ID No. 109 & 110 |
| 421379 | Y15221 | Hs.103982 | small inducible cytokine subfamily B(Cy | Seq ID No. 111 & 112 |
| 426028 | NM_001110 | Hs.172028 | a disintegrin and metalloproteinase doma | Seq ID No. 113 & 114 |
| 417079 | U65590 | Hs.81134 | interleukin 1 receptor antagonist | Seq ID No. 115 & 116 |
| 401093 | | | C12000586*: gi\|6330167\|dbj\|BAA86477.1\|(A | Seq ID No. 117-119 |
| 422247 | U18244 | Hs.113602 | solute carrier family 1 (high affinity a | Seq ID No. 120 & 121 |
| 449722 | BE280074 | Hs.23960 | cyclin B1 | Seq ID No. 122 & 123 |
| 423673 | BE003054 | Hs.1695 | matrix metalloproteinase 12 (macrophage | Seq ID No. 124 & 125 |
| 418478 | U38945 | Hs.1174 | cyclin-dependent kinase inhibitor 2A (me | Seq ID No. 126-133 |
| 439606 | W79123 | Hs.58561 | G protein-coupled receptor 87 | Seq ID No. 134 & 135 |
| 418543 | NM_005329 | Hs.85962 | hyaluronan synthase 3 | Seq ID No. 136 & 137 |
| 418678 | NM_001327 | Hs.167379 | cancer/testis antigen (NY-ESO-1) | Seq ID No. 138-141 |
| 428182 | BE386042 | Hs.293317 | ESTs, Weakly similar to GGC1_HUMAN G ANT | Seq ID No. 142 & 143 |
| 427335 | AA448542 | Hs.251677 | G antigen 7B | Seq ID No. 144 & 145 |
| 409420 | Z15008 | Hs.54451 | laminin, gamma 2 (nicein (100 kD), kalini | Seq ID No. 146 & 147 |
| 404440 | | | NM_021048: Homo sapiens melanoma antigen, | Seq ID No. 148 & 149 |
| 433091 | Y12642 | Hs.3185 | lymphocyte antigen 6 complex, locus D | Seq ID No. 150 & 151 |
| 408380 | AF123050 | Hs.44532 | diubiquitin | Seq ID No. 152 & 153 |
| 409893 | AW247090 | Hs.57101 | minichromosome maintenance deficient (S. | Seq ID No. 154 & 155 |
| 424905 | NM_002497 | Hs.153704 | NIMA (never in mitosis gene a)-related k | Seq ID No. 156 & 157 |
| 438817 | AI023799 | | ESTs | Seq ID No. 158 |
| 421478 | AI683243 | Hs.97258 | ESTs, Moderately similar to S29539 ribos | Seq ID No. 159 & 160 |
| 431515 | NM_012152 | Hs.258583 | EDG-7 (endothelial differentiation, lys | Seq ID No. 161 & 162 |
| 433159 | AB035898 | Hs.150587 | kinesin-like protein 2 | Seq ID No. 163 & 164 |
| 432441 | AW292425 | Hs.163484 | ESTs | Seq ID No. 165 |
| 425726 | AF085808 | Hs.159330 | uroplakin 3 | Seq ID No. 166 & 167 |
| 448045 | AJ297436 | Hs.20166 | prostate stem cell antigen | Seq ID No. 168 & 169 |
| 431846 | BE019924 | Hs.271580 | uroplakin 1B | Seq ID No. 170 & 171 |
| 437044 | AL035864 | Hs.69517 | differentially expressed in Fanconi's an | Seq ID No. 172 & 173 |
| 444381 | BE387335 | Hs.283713 | ESTs, Weakly similar to S64054 hypotheti | Seq ID No. 174 & 175 |
| 400303 | AA242758 | Hs.79136 | LIV-1 protein, estrogen regulated | Seq ID No. 176 & 177 |
| 452747 | BE153855 | Hs.61460 | Ig superfamily receptor LNIR | Seq ID No. 178 & 179 |
| 400297 | AI127076 | Hs.306201 | hypothetical protein DKFZp564O1278 | Seq ID No. 180 & 181 |
| 428484 | AF104032 | Hs.184601 | solute carrier family 7 (cationic amino | Seq ID No. 182 & 183 |
| 429211 | AF052693 | Hs.198249 | gap junction protein, beta 5 (connexin 3 | Seq ID No. 184 & 185 |
| 417389 | BE260964 | Hs.82045 | midkine (neurite growth-promoting factor | Seq ID No. 186 & 187 |
| 445537 | AJ245671 | Hs.12844 | EGF-like-domain, multiple 6 | Seq ID No. 188 & 189 |
| 423961 | D13666 | Hs.136348 | periostin (OSF-2os) | Seq ID No. 190 & 191 |
| 417433 | BE270266 | Hs.82128 | 5T4 oncofetal trophoblast glycoprotein | Seq ID No. 192 & 193 |
| 444781 | NM_014400 | Hs.11950 | GPI-anchored metastasis-associated protein homolog | Seq ID No. 194 & 195 |
| 430486 | BE062109 | Hs.241551 | chloride channel, calcium activated, family member 2 | Seq ID No. 196 & 197 |
| 425650 | NM_001944 | Hs.1925 | desmoglein 3 (pemphigus vulgaris antigen) | Seq ID No. 198 & 199 |
| 409103 | AF251237 | Hs.112208 | XAGE-1 protein | Seq ID No. 200 & 201 |
| 129404 | AI267700 | | ESTs | Seq ID No. 202 |
| 403047 | | | NM_005656*: Homo sapiens transmembrane protease | Seq ID No. 203 & 204 |
| 439738 | BE246502 | Hs.9598 | sema domain, immunoglobulin domain (Ig), transmem | Seq ID No. 205 & 206 |

Pkey: Unique Eos probeset identifier number
ExAccn: Exemplar Accession number, Genbank accession number
UnigeneID: Unigene number
Unigene Title: Unigene gene title
Seq ID No.: Sequence Identification Number linking information in Table 12A to sequences in Table 13

TABLE 12B

| Pkey | CAT Number | Accession |
|---|---|---|
| 413804 | 1390710_1 | T64682 BE168190 BE168256 |
| 427239 | 27647_1 | BE270447 AW409921 BE207288 BE207170 D56355 BE263223 BE408171 BE262243 BE392439 BE292738 BE261776 |
| | | BE314300 BE267719 BE268715 BE513876 BE295291 BE297066 AA210923 BE407519 H51344 BE622905 AW248281 |
| | | AW250313 T19021 AA355115 AA316879 BE269633 BE621936 AA290724 AA380031 AA993616 BE301331 |
| | | W79220 H73020 AW797850 BE274611 BE314315 BE542407 BE562063 BE312868 BE278817 BE252503 AA426470 |
| | | M91670 NM_014501 BE264533 AI884863 AI884942 AI678077 AA829937 AI869333 AW273060 AA906924 |
| | | AI571293 AW172642 AA582906 AW674596 AW300936 AW615753 AW615587 AW665495 AI859296 AA594105 |
| | | AA928110 AI038859 AI589898 AA828316 AA938955 AA283711 AW170665 BE047759 AW732128 AI015067 AW070748 |
| | | AW248955 AA975490 AA211097 AI826097 AA826730 AA994072 AI367867 W74508 AI086474 AA827720 AI922589 |
| | | AW000868 W58033 AA991461 BE048131 M91218 AA876186 AA661759 AI123879 AI089508 AW272915 AI566244 |
| | | AW245061 AA688299 AW250988 AI953468 AI891144 AW089131 AI471577 AA293354 AA464019 BE044549 |
| | | T29587 AW956171 BE537716 BE269400 AA918328 AI536087 AA969243 AW075033 BE139361 AI340589 |
| | | AI250128 AI247038 BE138953 AW075177 AI307208 BE049086 AW302327 AI054335 AI345565 AI334881 AI252075 |
| | | AI254494 AW075006 AW302733 AW302738 AI054057 AI054217 AI054172 AI054302 AI053722 AI054060 AI054079 |
| | | AW075181 AI307473 AI312145 AI340734 AI334909 AW071374 BE138502 AW074809 AW301901 AI251662 AI307559 |
| | | BE139228 AI254764 AW073049 AI251264 AI802837 AW271867 AI307442 AW075100 AW073456 AW072496 |
| | | AI270787 AW271039 AW071307 AI610913 AW071289 AI251232 AI251262 AW073656 AW072901 AI307493 |
| | | AI255068 AI251289 AI252160 AI271496 AI252427 AW073469 AW071420 AI270156 AI252926 AI252839 |
| | | AI252868 AW072520 AW073433 AI340643 AI802854 AI334733 AI583896 AW071311 AI802853 AI289711 |
| | | AI345036 AW072513 AI348921 AI307478 AI305762 BE139315 AW271034 AI334886 AI340619 AW470478 |
| | | BE139260 BE138965 AW302085 BE041872 AW268964 |
| 432222 | 343347_1 | AI204995 AW827539 AW969908 AW440776 AA528756 |
| 436608 | 42361_3 | AA628980 AI126603 BE504035 |
| 438817 | 465592_1 | AI023799 AA826307 AI683094 AI307373 AI870547 AW979007 |
| 444163 | 593658_1 | AI126098 AI184746 AI148521 |
| 456034 | 142696_1 | AW450979 AA136653 AA136656 AW419381 AA984358 AA492073 BE168945 |
| | | AA809054 AW238038 BE011212 BE011359 BE011367 |
| | | BE011368 BE011362 BE011215 BE011365 BE011363 |

Pkey: Unique Eos probeset identifier number
CAT number: Gene cluster number
Accession: Genbank accession numbers

TABLE 12C

| Pkey | Ref | Strand | Nt_position |
|---|---|---|---|
| 400844 | 9188605 | Plus | 24746-24872, 25035-25204 |
| 401093 | 8516137 | Minus | 22335-23166 |
| 402305 | 7328724 | Plus | 40832-41362 |
| 404440 | 7528051 | Plus | 80430-81581 |
| 404875 | 9801324 | Plus | 96588-96732, 97722-97831 |
| 405033 | 7107731 | Minus | 142358-142546 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 13

| | |
|---|---|
| Seq ID NO: 1 DNA sequence: | |
| Nucleic Acid Accession #: NM_002205 | |
| Coding sequence: 24 . . . 3173 | |
| 1 11 21 31 41 51 | |
| \| \| \| \| \| \| | |
| CACGACAGGG AACAGCGGGC GCTATGGGGA GCCGGACGCC AGAGTCCCCT CTCCACGCCG | 60 |
| TGCAGCTGCC CTGCCCCCCC CCGCGCCCAC CCCCGCTCGT GCCGCTGCTG TTGCTGCTCG | 120 |
| TGCC0CCGCC ACCCAGCGTC GGGGGCTTCA ACTTAGACGC GGAGGCCCCA GCAGTACTCT | 180 |
| CGGGGCCCCC CCCCTCCTTC TTCGGATTCT CAGTGGAGTT TTACCGGCCG GGAACAGACG | 240 |
| CCCTCAGTGT GCTGGTGCCA CCACCCAAGG CTAATACCAG CCAGCCAGGA GTGCTGCAGG | 300 |
| GTGCTUCTGT CTACCTCTGT CCTTGGGGTG CCAGCCCCAC ACAGTGCACC CCCATTGAAT | 360 |
| TTGACAGCAA AGGCTCTCGG CTCCTGGAGT CCTCACTGTC CACCTCAGAG GGAGAGGAGC | 420 |
| CTGTGGAGTA CAAGTCCTTG CAGTGGTTCG GGGCAACAGT TCGAGCCCAT GGCTCCTCCA | 480 |
| TCTTGGCATG CGCTCCACTG TACAGCTGGC GCACACAGAA CGAGCCACTG AGCGACCCCG | 540 |
| TGGGCACCTC CTACCTCTCC ACAGATAACT TCACCCGAAT TCTGGAGTAT GCACCCTGCC | 600 |
| GCTCAGATTT CAGCTGGGCA GCAGGACAGG GTTACTGCCA AGGAGGCTTC ACTGCCGACT | 660 |
| TCACCAAGAC TGGCCGTGTG GTTTTAGGTG GACCAGGAAG CTATTTCTGG CAAGGCCAGA | 720 |
| TCCTGTCTGC CACTCAGGAG CAGATTGCAG AATCTTATTA CCCCGAGTAC CTGATCAACC | 780 |
| TGGTTCAGGG GCAGCTGCAG ACTCGCCAGG CCAGTTCCAT CTATGATGAC AGCTACCTAG | 840 |
| GATACTCTGT GUCTGTTGGT GAATTCAGTG GTGATGACAC AGAAGACTTT GTTGCTGGTG | 900 |
| TGCCCAAAGG GAACCTCACT TACCGCTATG TCACCATCCT TAATGGCTCA GACATICCAT | 960 |
| CCCTCTACAA CTTCTCAGGG GAACAGATGG CCTCCTACTT TGGCTATGCA GTGGCCGCCA | 1020 |
| CAGACGTCAA TGGGGACGGG CTGGATGACT TGCTGGTGGG GGCACCCCTG CTCATGGATC | 1080 |
| GGACCCCTGA CGGGCGGCCT CAGGAGGTGG GCAGGGTCTA CGTCTACCTG CACCACCCAG | 1140 |
| CCGGCATAGA GCCCACGCCC ACCCTTACCC TCACTGGCCA TGATGAGTTT GGCCGATTTG | 1200 |
| GCAGCTCCTT GACCCCCCTG GGGGACCTGG ACCAGGATGG CTACAATGAT GTGGCCATCG | 1260 |
| GGGCTCCCTT TGGTGGGGAG ACCCAGCAGG GAGTAGTGTT TGTATTTCCT GGGGGCCCAG | 1320 |
| GAGGGCTGGG CTCTAAGCCT TCCCAGGTTC TGCAGCCCCT GTGGGCAGCC AGCCACACCC | 1380 |
| CAGACTTCTT TGGCTCTGCC CTTCGAGGAG GCCGAGACCT GGATGGCAAT GGATATCCTG | 1440 |
| ATCTGATTGT GGGGTCCTTT GGTGTGGACA AGGCTGTGGT ATACAGGGGC CGCCCCATCG | 1500 |
| TGTCCGCTAG TGCCTCCCTC ACCATCTTCC CCGCCATGTT CAACCCAGAG GAGCGGAGCT | 1560 |
| GCAGCTTAGA GGGGAACCCT GTGGCCTGCA TCAACCTTAG CTTCTGCCTC AATGCTTCTG | 1620 |
| GAAAACACGT TGCTGACTCC ATTGGTTTCA CAGTGGAACT TCAGCTGGAC TGGCAGAAGC | 1680 |
| AGAAGGGAGG GGTACGGCGG GCACTGTTCC TGGCCTCCAG GCAGGCAACC CTGACCCAGA | 1740 |
| CCCTGCTCAT CCAGAATGGG GCTCGAGAGG ATTGCAGAGA GATGAAGATC TACCTCAGGA | 1800 |
| ACGAGTCAGA ATTTCGAGAC AAACTCTCGC CGATTCACAT CGCTCTCAAC TTCTCCTTGG | 1860 |
| ACCCCCAAGC CCCAGTGGAC AGCCACGGCC TCAGGCCAGC CCTACATTAT CAGAGCAAGA | 1920 |
| GCCGGATAGA GGACAAGGCT CAGATCTTGC TGGACTGTGG AGAAGACAAC ATCTGTGTGC | 1980 |
| CTGACCTGCA GCTGGAAGTG TTTGGGGAGC AGAACCATGT GTACCTGGGT GACAAGAATG | 2040 |
| CCCTGAACCT CACTTTCCAT GCCCAGAATG TGGGTGAGGG TGGCGCCTAT GAGGCTGAGC | 2100 |
| TTCGGGTCAC CGCCCCTCCA GAGGCTGAGT ACTCAGGACT CGTCAGACAC CCAGGGAACT | 2160 |
| TCTCCAGCCT GAGCTGTGAC TACTTTGCCG TGAACCAGAG CCGCCTGCTG GTGTGTGACC | 2220 |
| TGGGCAACCC CATGAAGGCA GGAGCCAGTC TGTGGGGTGG CCTTCGGTTT ACAGTCCCTC | 2280 |
| ATCTCCGGGA CACTAAGAAA ACCATCCAGT TTGACTTCCA GATCCTCAGC AAGAATCTCA | 2340 |
| ACAACTCGCA AAGCGACGTG GTTTCCTTTC GGCTCTCCGT GGAGGCTCAG GCCCAGGTCA | 2400 |
| CCCTGAACGG TGTCTCCAAG CCTGAGGCAG TGCTATTCCC AGTAAGCGAC TGGCATCCCC | 2460 |
| GAGACCAGCC TCAGAAGGAG GAGGACCTGG GACCTGCTGT CCACCATGTC TATGAGCTCA | 2520 |
| TCAACCAAGG CCCCAGCTCC ATTAGCCAGG GTGTGCTGGA ACTCAGCTGT CCCCAGGCTC | 2580 |
| TGGAAGGTCA GCAGCTCCTA TATGTGACCA GAGTTACGGG ACTCAACTGC ACCACCAATC | 2640 |
| ACCCCATTAA CCCAAAGGGC CTGGAGTTGG ATCCCGAGGG TTCCCTGCAC CACCAGCAAA | 2700 |
| AACGGGAAGC TCCAAGCCGC AGCTCTGCTT CCTCGGGACC TCAGATCCTG AAATGCCCGG | 2760 |
| AGGCTGAGTG TTTCAGGCTG CGCTGTGAGC TCGGGCCCCT GCACCAACAA GAGAGCCAAA | 2820 |
| GTCTGCAGTT GCATTTCCGA GTCTGGGCCA AGACTTTCTT GCACCGGGAG CACCAGCCAT | 2880 |
| TTAGCCTGCA GTGTGAGGCT GTGTACAAAG CCCTGAAGAT GCCCTACCGA ATCCTGCCTC | 2840 |
| GGCAGCTGCC CCAAAAAGAG CGTCAGGTGG CCACAGCTGT GCAATGGACC AAGGCAGAAG | 3000 |
| GCAGCTATGG CGTCCCACTG TGGATCATCA TCCTAGCCAT CCTGTTTGGC CTCCTGCTCC | 3060 |
| TAGGTCTACT CATCTACATC CTCTACAAGC TTGGATTCTT CAAACGCTCC CTCCCATATG | 3120 |
| GCACCGCCAT GGAAAAAGCT CAGCTCAAGC CTCCAGCCAC CTCTGATGCC TGAGTCCTCC | 3180 |
| CAATTTCAGA CTCCCATTCC TGAAGAACCA GTCCCCCCAC CCTCATTCTA CTGAAAAGGA | 3240 |
| GGGGTCTGGG TACTTCTTGA AGGTGCTGAC GGCCAGGGAG AAGCTCCTCT CCCCAGCCCA | 3300 |
| GAGACATACT TGAAGGGCCA GAGCCAGGGG GGTGAGGAGC TGGGGATCCC TCCCCCCCAT | 3360 |
| GCACTGTGAA GGACCCTTGT TTACACATAC CCTCTTCATG GATGGGGGAA CTCAGATCCA | 3420 |
| GGGACAGAGG CCCAGCCTCC CTGAAGCCTT TGCATTTTGG AGAGTTTCCT GAAACAACTG | 3480 |
| GAAAGATAAC TAGGAAATCC ATTCACAGTT CTTTGGGCCA GACATGCCAC AAGGACTTCC | 3540 |
| TGTCCAGCTC CAACCTGCAA AGATCTGTCC TCAGCCTTGC CAGAGATCCA AAAGAAGCCC | 3600 |
| CCAGTAAGAA CCTGGAACTT GGGGAGTTAA GACCTGGCAG CTCTGGACAG CCCCACCCTG | 3660 |
| GTGGGCCAAC AAAGAACACT AACTATGCAT GGTGCCCCAG GACCAGCTCA GGACAGATGC | 3720 |
| CACAAGGATA GATGCTGGCC CAGGGCCAGA GCCCAGCTCC AAGGGGAATC AGAACTCAAA | 3780 |
| TGGGGCCAGA TCCAGCCTGG GGTCTGGAGT TGATCTGGAA CCCAGACTCA GACATTGGCA | 3840 |
| CCAATCCAGG CAGATCCAGG ACTATATTTG GGCCTGCTCC AGACCTGATC CTGGAGGCCC | 3900 |
| AGTTCACCCT GATTTAGGAG AAGCCAGGAA TTTCCCAGGA CCTGAAGGGG CCATGATGGC | 3960 |
| AACAGATCTG GAACCTCAGC CTGGCCAGAC ACAGGCCCTC CCTGTTCCCC AGAGAAAGGG | 4020 |
| GAGCCCACTG TCCTGGGCCT GCAGAATTTG GGTTCTGCCT GCCAGCTGCA CTGATGCTGC | 4080 |
| CCCTCATCTC TCTGCCCAAC CCTTCCCTCA CCTTGGCACC AGACACCCAG GACTTATTTA | 4140 |
| AACTCTGTTG CAAGTGCAAT AAATCTGACC CAGTGCCCCC ACTGACCAGA ACTAGAAAAA | 4200 |
| AAAA | |
| Seq ID NO: 2 Protein sequence: | |
| Protein Acceecion #: NP_002196.1 | |
| 1 11 21 31 41 51 | |
| \| \| \| \| \| \| | |
| MGSRTPESPL NAVQLRWGPR RRPPLVPLLL LLVPPPPRVG GFNLDAEAPA VLSGPPGSFP | 60 |
| GFSVEFYRPG TDGVSVLVGA PKANTSQPGV LQGGAVYLCP WGASPTQCTP IEPDSKGSRL | 120 |
| LESSLSSSEG EEPVEYKSLQ WFGATVRARG SSILACAPLY SWRTEKEPLS DPVGTCYLST | 180 |
| DNPTRILEYA PCRSDFSWAA GQGYCQGGPS AEPTKTGRVV LGGPGSYFNQ GQILSATQEQ | 240 |
| IAESYYPEYL INLVQGQLQT RQASSIYDDS YLGYSVAVGE PSGDDTEDPV AGVPKGNLTY | 300 |
| GYVTILNGSD INSLYNPGGE QMASYFGYAV AATDVNGDGL DDLLVGAPLL MDRTPDGRPQ | 360 |
| EVGRVYVYLQ HPAGIEPTPT LTLTGHDEFG RFGSSLTPLG DLDQDGYNDV AIGAPEGGET | 420 |
| QQGVVFVPPG GPGGLGSEPS QVLQPLWAAS NTPDFFGSAL RGGRGLDGNG YPDLIVGSFG | 480 |
| VDKAVVYRGR PIVSASASLT IFPANPNPEE RSCSLEGNPV ACINLSFCLN ASGENVADSI | 540 |
| GPTVELQLDW QKQKGGVRRA LFLASRQATL TQTLLIQNGA REGCREMEIY LRNESEFRDK | 600 |
| LSPINIALNF SLDPQAPVDS NGLRPALHYQ SKSRIEDKAQ ILLGCCEDNI CVPDLQLEVF | 660 |
| GEQNHVYLGD KNALNLTFNA QNVGEGGAYE AELRVTAPPE AEYSGLVRNP GNFSSLSCDY | 720 |
| EAVNQSRLLV CDLGNPMKAG ASLWGGLRFT VPNLRDTKKT IQFDFQILSK NLNNSQSDVV | 780 |
| SFRLSVEAQA QVTLNGVSKP EAVLFPVSDW NPRDQPQKEE DLGPAVNNVY ELINQGPSSI | 840 |
| SQGVLELSCP QALEGQQLLY VTRVTGLNCT TNHPINPKGL ELDPEGSLNH QQKREAPSRS | 900 |
| SASSGPQILK CPEAECFRLR CELGPLNQQE SQSLQLNPRV WAKTFLQREN QPFSLQCEAV | 960 |
| YKALKNPYRI LPRQLPQKER QVATAVQWTK AEGSYGVPLW IIILAILFGL LLLGLLIYIL | 1020 |
| YKLGPPKRSL PYGTANEKAQ LKPPATSDA | |
| Seq ID NO: 3 DNA sequence: | |
| Nucleic Acid Accession #: NM_002425 | |
| Coding sequence: 23 . . . 1453 | |
| 1 11 21 31 41 51 | |
| \| \| \| \| \| \| | |
| AAAGAAGGTA AGGGCAGTGA GAATGATGCA TCTTGCATTC CTTGTGCTGT TGTGTCTGCC | 60 |
| AGTCTGCTCT GCCTATCCTC TGAGTGGGGC AGCAAAAGAG GAGGACTCCA ACAAGGATCT | 120 |
| TGCCCAGCAA TACCTAGAAA AGTACTACAA CCTCGAAAAG GATGTGAAAC AGTTTAGAAG | 180 |
| AAAGGACAGT AATCTCATTG TTAAAAAAAT CCAAGGAATG CAGAAGTTCC TTGGGTTGGA | 240 |
| GGTGACAGGG AAGCTAGACA CTGACACTCT GGAGGTGATG CGCAAGCCCA GGTGTGGAGT | 300 |
| TCCTGACGTT GGTCACTTCA GCTCCTTTCC TGGCATGCCG AAGTGGAGGA AAACCCACCT | 360 |
| TACATACAGG ATTGTGAATT ATACACCAGA TTTGCCAAGA GATGCTGTTG ATTCTGCCAT | 420 |
| TGAGAAAGCT CTGAAAGTCT GGGAAGAGGT GACTCCACTC ACATTCTCCA GGCTGTATGA | 480 |
| AGGAGAGGCT GATATAATGA TCTCTTTCGC AGTTAAAGAA CATGGAGACT TTTACTCTTT | 540 |
| TGATGGCCCA GGACACAGTT TGGCTCATGC CTACCCACCT GGACCTGGGC TTTATGGAGA | 600 |
| TATTCACTTT GATGATGATG AAAAATGGAC AGAAGATGCA TCAGGCACCA ATTTATTCCT | 660 |
| CGTTGCTGCT CATGAACTTG GCCACTCCCT GGGGCTCTTT CACTCAGCCA ACACTGAAGC | 720 |
| TTTGATGTAC CCACTCTACA ACTCATTCAC AGAGCTCGCC CAGTTCCGCC TTTCGCAAGA | 780 |
| TGATGTGAAT GGCATTCAGT CTCTCTACGG ACCTCCCCCT GCCTCTACTG AGCAACCCCT | 840 |
| GGTGCCCACA AAATCTGTTC CTTCGGGATC TGAGATGCCA GCCAAGTGTG ATCCTGCTTT | 900 |
| GTCCTTCGAT GCCATCAGCA CTCTGAGGGG AGAATATCTG TTCTTTAAAG ACAGATATTT | 960 |
| TTGGCGAAGA TCCCACTGGA ACCCTGAACC TGAATTTCAT TTGATTTCTG CATTTTGGCC | 1020 |
| CTCTCTTCCA TCATATTTGG ATGCTGCATA TGAAGTTAAC ACCAGGGACA CCGTTTTTAT | 1080 |
| TTTTAAAGGA AATGAGTTCT GGGCCATCAG AGGAAATGAG GTACAAGCAG GTTATCCAAG | 1140 |
| AGGCATCCAT ACCCTGGGTT TTCCTCCAAC CATAAGGAAA ATTGATGCAG CTGTTTCTGA | 1200 |
| CAAGGAAAAG AAGAAAACAT ACTTCTTTGC AGCGGACAAA TACTGGAGAT TTGATGAAAA | 1260 |
| TAGCCAGTCC ATGGAGCAAG GCTTCCCTAG ACTAATAGCT GATGACTTTC CAGGAGTTGA | 1320 |
| GCCTAAGGTT GATGCTGTAT TACAGGCATT TGGATTTTTC TACTTCTTCA GTGGATCATC | 1380 |
| ACAGTTTGAG TTTGACCCCA ATGCCAGGAT GGTGACACAC ATATTAAAGA GTAACAGCTG | 1440 |
| GTTACATTGC TAGGCGAGAT AGGGGGAAGA CAGATATGGG TGTTTTTAAT AAATCTAATA | 1500 |
| ATTATTCATC TAATGTATTA TGAGCCAAAA TGGTTAATTT TTCCTGCATG TTCTGTGACT | 1560 |
| GAAGAAGATG AGCCTTGCAG ATATCTGCAT GTGTCATGAA GAATGTTTCT GGAATTCTTC | 1620 |
| ACTTGCTTTT GAATTGCACT GAACAGAATT AAGAAATACT CATGTGCAAT AGGTGAGAGA | 1680 |
| ATGTATTTTC ATAGATGTGT TATTACTTCC TCAATAAAAA GTTTTATTTT GGGCCTGTTC | 1740 |
| CTT | |
| Seq ID NO: 4 Protein sequence: | |
| Protein Accession #: NP_002416 | |
| 1 11 21 31 41 51 | |
| \| \| \| \| \| \| | |
| MMNLAFLVLL CLPVCSAYPL SGAAKEEDSN KDLAQQYLEK YYNLEKDVKQ FRRKDSNLIV | 60 |
| KKIQGMQKFL GLEVTGKLDT DTLEVNRKPR CGVPDVGNFS SFPGMPKWRK TELTYRIVNY | 120 |
| TPDLPRDAVD SAIEKALKVW EEVTPLTFSR LYEGEADIMI SFAVKENGDF YSFDGPGNSL | 180 |
| ANAYPPGPGL YGDINFDDDE KWTEDASGTN LFLVAANELG NSLGLFNSAN TEALMYPLYN | 240 |
| SFTELAQFRL SQDDVNGIQS LYGPPPASTE EPLVPTKSVP SGSEMPAKCD PALSFDAIST | 300 |
| LRGEYLFFKD RYFNRRSNWN PEPEFNLISA FWPSLPSYLD AAYEVNSRDT VFIFKGNEFW | 360 |
| AIRGNEVQAG YPRGINTLGF PPTIRKIDAA VSGKEKKKTY FFAADKYWRF DENSQSMEQG | 420 |
| FGNLIADDEP GVEPKVDAVL GAFGFFYFFS GSSQPEFDPN ANMVTHILKS NSWLNC | |
| Seq ID NO: 5 DNA sequence: | |
| Nucleic Acid Accession #: NN_002421 | |
| Coding sequence: 72 . . . 1481 | |
| 1 11 21 31 41 51 | |
| \| \| \| \| \| \| | |
| GGGATATTGC AGTAGCAAGA GGCTGGGAAG CCATCACTTA CCTTGCACTG AGAAAGAAGA | 60 |
| CAAACCCCAC TATCCACAGC TTTCCTCCAC TGCTGCTGCT GCTGTTCTGG GGTGTGGTGT | 120 |
| CTCACAGCTT CCCAGCGACT CTAGAAACAC AAGAGCAAGA TGTGGACTTA GTCCAGAAAT | 180 |
| ACCTGGAAAA ATACTACAAC CTGAAGAATG ATGGGAGGCA AGTTGAAAAG CGGAGAAATA | 240 |
| GTGGCCCAGT GGTTGAAAAA TTGAAGCAAA TGCAGCAATT CTTTGGGCTG AAAGTGACTC | 300 |
| GGAAACCAGA TGCTGAAACC CTGAACGTGA TGAAGCACCC CAGATGTGGA GTGCCTGATG | 360 |
| TCGCTCAGTT TCTCCTCACT GACGGGAACC CTCGCTGGGA GCAAACACAT CTGACCTACA | 420 |
| GGATTGAAAA TTACACGCCA GATTTGCCAA GAGCAGATGT GGACCATGCC ATTGAGAAAG | 480 |
| CCTTCCAACT CTGGAGTAAT GTCACACCTC TGACATTCAC CAAGGTCTCT GAGGGTCAAG | 540 |
| CAGACATCAT GATATCTTTT GTCAGGGGAG ATCATCGGGA CAACTCTCCT TTTGATGGAC | 600 |
| CTGGAGGAAA TCTTGCTCAT GCTTTTCAAC CAGGCCCAGG TATTGGAGGG GATGCTCATT | 660 |
| TTGATGAAGA TGAAAGGTGG ACCAACAATT TCAGAGAGTA CAACTTACAT CGTGTTGCGG | 720 |
| CTCATGAACT CGGCCATTCT CTTGGACTCT CCCATTCTAC TGATATCGGG GCTTTGATGT | 780 |
| ACCCTAGCTA CACCTTCAGT GGTGATGTTC AGCTAGCTCA GGATGACATI GATGGCATCC | 840 |
| AAGCCATATA TGGACGTTCC CAAAATCCTG TCCAGCCCAT CGGCCCACAA ACCCCAAAAG | 900 |
| CGTGTGACAG TAAGCTAACC TTTGATGCTA TAACTACGAT TCGGGGAGAA GTGATGTTCT | 960 |
| TTAAAGACAG ATTCTACATG CGCACAAATC CCTTCTACCC GGAAGTTGAG CTCAATTTCA | 1020 |
| TTTCTGTTTT CTGGCCACAA CTGCCAAATG GGCTTGAAGC TGCTTACGAA TTTGCCGACA | 1080 |
| GAGATGAAGT CCGGTTTTTC AAAGGGAATA ACTACTGGGC TGTTCAGGGA CAGAATGTGC | 1140 |
| TACACGGATA CCCCAAGGAC ATCTACAGCT CCTTTGGCTT CCCTAGAACT GTGAAGCATA | 1200 |
| TCGATGCTGC TCTTTCTGAG GAAAACACTG GAAAAACCTA CTTCTTTGTT GCTAACAAAT | 1260 |
| ACTGGAGGTA TGATGAATAT AAACGATCTA TGGATCCAGG TTATCCCAAA ATGATAGCAC | 1320 |
| ATGACTTTCC TGGAATTGGC CACAAAGTTG ATGCAGTTTT CATGAAAGAT GGATTTTTCT | 1380 |
| ATTTCTTTCA TGGAACAAGA CAATACAAAT TTGATCCTAA AACGAAGAGA ATTTTGACTC | 1440 |
| TCCAGAAAGC TAATAGCTGG TTCAACTGCA GGAAAAATTG AACATTACTA ATTTGAATGG | 1500 |
| AAAACACATG GTGTGAGTCC AAAGAAGGTG TTTTCCTGAA GAACTGTCTA TTTTCTCAGT | 1560 |
| CATTTTTAAC CTCTAGAGTC ACTGATACAC AGAATATAAT CTTATTTATA CCTCAGTTTG | 1620 |
| CATATTTTTT TACTATTTAG AATGTAGCCC TTTTTGTACT GATATAATTT AGTTCCACAA | 1680 |
| ATGGTGGGTA CAAAAAGTCA AGTTTGTGGC TTATGGATTC ATATAGGCCA GAGTTGCAAA | 1740 |
| GATCTTTTCC AGACTATGCA ACTCTGACGT TGATGCCAGA GAGCAGCTTC AGTGACAAAC | 1800 |
| ATATCCTTTC AAGACAGAAA GAGACAGGAG ACATGAGTCT TTGCCGGAGG AAAAGCAGCT | 1860 |
| CAAGAACACA TGTGCAGTCA CTGGTGTCAC CCTGGATAGG CAAGGGATAA CTCTTCTAAC | 1920 |
| ACAAAATAAG TGTTTTATGT TTGGAATAAA GTCAACCTTG TTTCTACTGT TTT | |
| Seq ID NO: 6 Protein sequence: | |
| Protein Accession 4: NP_002412 | |
| 1 11 21 31 41 51 | |
| \| \| \| \| \| \| | |
| MHSPPPLLLL LFWGVVSHSF PATLETQEQD VGLVQKYLEK YYNLKNGGRG VEKNRNSGPV | 60 |
| VEKLKQMQEP FGLKVTGKPD AETLKVMKQP RCGVPGVAQF VLTEGNPRWE QTNLTYNTEN | 120 |
| YTPGLPNAGV GNAIEKAFQL WSNVTPLTFT KVSEGQAGIM ISFVRGGHND NSPPGGPGGN | 180 |
| LANAFQPGPG IDGGAHEDED ENWTNNPNNY NLHRVAANNL GNSLGLSNST GIDALMYPSY | 240 |
| TFSGGVQLAQ GGIDGIQAIY GNSQNPVQPI DPQTPKACGS KLTFGAITTI NDEVNFFKGN | 300 |
| FYMRTNPFYP EVELNFISVF WPQLPNGLNA AYEFAGRGSV RFFKGNKYWA VQGQNVLHGY | 360 |
| PKDIYSSFGP PNTVKNIDAA LSEENTGKTY FFVANKYWRY GEYKESMGPG YPKMIANGFP | 420 |
| GIDNKVGAVF MEGGEFYPEN GTRQYKFGPK TKRILTLQKA NSWFNCNKN | |
| Seq ID NO: 7 DNA sequence: | |
| Nucleic Acid Accession 4: NM_014331 | |
| Coding sequence: 1 . . . 1506 | |
| 1 11 21 31 41 51 | |
| \| \| \| \| \| \| | |
| ATGGTCAGAA AGCCTGTTGT GTCCACCATC TCCAAAGGAG GTTACCTGCA GGGAAATGTT | 60 |
| AACGGGAGGC TGCCTTCCCT GGGCAACAAG GAGCCACCTG GGCAGGAGAA AGTGCAGCTG | 120 |
| AAGAGGAAAG TCACTTTACT GAGGGGAGTC TCCATTATCA TTGGCACCAT CATTGGAGCA | 180 |
| GGAATCTTCA TCTCTCCTAA GGGCGTGCTC CAGAACACGG GCAGCGTGGG CATGTCTCTG | 240 |
| ACCATCTGGA CGGTGTGTGG GGTCCTGTCA CTATTTGGAG CTTTGTCTTA TGCTGAATTG | 300 |
| GGAACAACTA TAAAGAAATC TGGAGGTCAT TACACATATA TTTTGGAAGT CTTTGGTCCA | 360 |
| TTACCAGCTT TTGTACGAGT CTGGGTGGAA CTCCTCATAA TACGCCCTGC AGCTACTGCT | 420 |
| GTGATATCCC TGGCATTTGG ACGCTACATT CTGGAACCAT TTTTTATTCA ATGTGAAATC | 480 |
| CCTGAACTTG CGATCAAGCT CATTACAGCT GTGGGCATAA CTGTAGTGAT GGTCCTAAAT | 540 |
| AGCATGAGTG TCAGCTGGAG CGCCCGGATC CAGATTTTCT TAACCTTTTG CAAGCTCACA | 600 |
| GCAATTCTGA TAATTATAGT CCCTGGAGTT ATGCAGCTAA TTAAAGGTCA AACGCAGAAC | 660 |
| TTTAAAGACG CGTTTTCAGG AAGAGATTCA AGTATTACGC GGTTGCCACT GGCTTTTTAT | 720 |
| TATGGAATGT ATGCATATGC TGGCTGGTTT TACCTCAACT TTGTTACTGA AGAAGTAGAA | 780 |
| AACCCTGAAA AAACCATTCC CCTTGCAATA TGTATATCCA TGGCCATTGT CACCATTGGC | 840 |
| TATGTGCTGA CAAATGTGGC CTACTTTACG ACCATTAATG CTGAGGAGCT GCTGCTTTCA | 900 |
| AATGCAGTGG CAGTGACCTT TTCTGAGCGG CTAGTGGCAA ATTTCTCATT ACCAGTTCCG | 960 |
| ATCTTTGTTG CCCTCTCCTG CTTTGGCTCC ATCAACGGTG GTGTGTTTGC TGTCTCCAGG | 1020 |
| TTATTCTATG TTGCGTCTCG AGAGGGTCAC CTTCCAGAAA TCCTCTCCAT GATTCATGTC | 1080 |
| CGCAAGCACA CTCCTCTACC AGCTGTTATT GTTTTGCACC CTTTGACAAT GATAATGCTC | 1140 |
| TTCTCTGGAG ACCTCGACAG TCTTTTGAAT TTCCTCAGTT TTGCCAGGTG GCTTTTTATT | 1200 |
| GGGCTGGCAG TTGCTGGGCT GATTTATCTT CGATACAAAT GCCCAGATAT GCATCGTCCT | 1260 |
| TTCAAGGTGC CACTGTTCAT CCCAGCTTTG TTTTCCTTCA CATGCCTCTT CATGGTTGCC | 1320 |
| CTTTCCCTCT ATTCGGACCC ATTTAGTACA GGGATTGGCT TCGTCATCAC TGTGACTGGA | 1380 |
| GTCCCTGCGT ATTATCTCTT TATTATATGG GACAAGAAAC CCAGGTGGTT TAGAATAATG | 1440 |
| TCAGAGAAAA TAACCAGAAC ATTACAAATA ATACTGGAAG TTGTACCAGA AGAAGATAAG | 1500 |
| TTATGAACTA ATGGACTTGA GATGTTGGGA ATCTGCCCAA GGGGAGACAC AAAATAGGGA | 1560 |
| TTTTTACTTC ATTTTCTGAA AGTCTAGAGA ATTACAACTT TGGTGATAAA CAAAAGGAGT | 1620 |
| CAGTTATTTT TATTGATATA TTTTAGCATA TTCGAACTAA TTTCTAAGAA ATTTAGTTAT | 1680 |
| AACTCTATGT AGTTATAGAA AGTGAATATG CAGTTATTCT ATGAGTCGCA CAATTCTTGA | 1740 |
| GTCTCTGATA CCTACCTATT GGGGTTAGGA GAAAAGACTA GACAATTACT ATGTGGTCAT | 1800 |
| TCTCTACAAC ATATGTTAGC ACGGCAAAGA ACCTTCAAAT TGAAGACTGA GATTTTTCTG | 1860 |
| TATATATGGG TTTTGTAAAG ATGGTTTTAC ACACTACAGA TGTGTATACT GTGAAAAGTG | 1920 |
| TTTTCAATTC TGAAAAAAAG CATACATCAT GATTATGGGA AAGAGGAGAG AAAGAAATTT | 1980 |
| ATTTTACATT GACATTGCAT TGCTTCCCCT TAGATACCAA TTTAGATAAC AAACACTCAT | 2040 |
| GCTTTAATGG ATTATACCCA GAGCACTTTG AACAAAGGTC AGTGGGGATT GTTGAATACA | 2100 |
| TTAAAGAAGA GTTTCTAGGG GCTACTGTTT ATGAGACACA TCCAGGAGTT ATGTTTAAGT | 2160 |
| AAAAATCCTT GAGAATTTAT TATGTCAGAT GTTTTTTCAT TCATTATCAG GAAGTTTTAG | 2220 |
| TTATCTGTCA TTTTTTTTTT TCACATCAGT TTGATCAGGA AAGTGTATAA CACATCTTAG | 2280 |
| AGCAAGAGTT AGTTTGGTAT TAAATCCTCA TTAGAACAAC CACCTGTTTC ACTAATAACT | 2340 |
| TACCCCTGAT GAGTCTATCT AAACATATGC ATTTTAAGCC TTCAAATTAC ATTATCAACA | 2400 |
| TGAGAGAAAT AACCAACAAA GAAGATGTTC AAAATAATAG TCCCATATCT GTAATCATAT | 2460 |
| CTACATGCAA TGTTAGTAAT TCTGAAGTTT TTTAAATTTA TGGCTATTTT TACACGATGA | 2520 |
| TGAATTTTGA CAGTTTGTGC ATTTTCTTTA TACATTTTAT ATTCTTCTGT TAAAATATCT | 2580 |
| CTTCAGATGA AACTGTCCAG ATTAATTAGG AAAAGGCATA TATTAACATA AAAATTGCAA | 2640 |
| AAGAAATGTC GCTGTAAATA AGATTTACAA CTGATGTTTC TAGAAAATTT CCACTTCTAT | 2700 |
| ATCTAGGCTT TGTCAGTAAT TTCCACACCT TAATTATCAT TCAACTTGCA AAAGAGACAA | 2760 |
| CTGATAAGAA GAAAATTGAA ATGAGAATCT GTGGATAAGT GTTTGTGTTC AGAAGATGTT | 2820 |
| GTTTTGCCAG TATTAGAAAA TACTGTGAGC CGGGCATGGT GGCTTACATC TGTAATCCCA | 2880 |
| GCACTTTGGG AGUCTGAGGG GGTGGATCAC CTGAGGTCGG GAGTTCTAGA CCAGCCTGAC | 2940 |
| CAACATGGAG AAACCCCATC TCTACTAAAA ATACAAAATT AGCTGGGCAT GGTGGGACAT | 3000 |
| GCTGGTAATC TCAGCTATTG AGGAGGCTGA GGCAGGAGAA TTGCTTGAAC CCGGGAGGCG | 3060 |
| GAGGTTGCAG TGAGCCAAGA TTGCACCACT GTACTCCAGC CTGGGTGACA AAGTCAGACT | 3120 |
| CCATCTCCAA AAAAAAAAAA AAAA | |
| Seq ID NO: 8 Protein sequence | |
| Protein Accession #: NP_055146 | |
| 1 11 21 31 41 51 | |
| \| \| \| \| \| \| | |
| NVRKPVVSTI SKGGYLQGNV NGRLPSLGNK EPPGQEKVQL KRKVTLLRGV SIIIDTIIDA | 60 |
| GIFTSPKGVL CNTGSVGMSL TIWTVCGVLS LFGALSYAEL GTTIKKSGGN YTYILEVFGP | 120 |
| LPAFVRVWVE LLIIRPAATA VISLANGRYT LEPPFIQCEI PELAIRLITA VGITVVNVLN | 180 |
| SMSVSNSARI QTFLTFCKLT AILIIIVPGV NQLIKGQTQN FKGAFSGRGS SITELPEAFY | 240 |
| YGNYAYAGWF YLNFVTEBVN NPEKTIPLAI CISNAIVTID YVLTNVAYFT TINANNLLLS | 300 |
| NAVAVTFSER LLGNFSLAVP IFVALSCFGS MNGGVFAVSN LFYVASREGN LPNILSMINV | 360 |
| RKHTPLPAVI VLNPLTMIML FSGGLGSLLN FLSFARNLFI GLAVAGLIYL RYKCPGMNRP | 420 |
| FKVPLFIPAL FSFTCLFNVA LSLYSGPFST GIDFVITLTG VPAYYLFIIN GERPEWERIN | 480 |
| SEKITRTLQI ILEVVPEEGK L | |
| Seq ID NO: 9 DNA sequence | |
| Nucleic Acid Accession #: FGENESH | |
| Coding sequence: 1 . . . 546 | |
| 1 11 21 31 41 51 | |
| \| \| \| \| \| \| | |
| ATGGCCTTGG GCAGCTCCGC CCCTGTGGCT TTGCAGGGTA ATGCCCACTT CCCTGCTGCT | 60 |
| TTCATGGCTG GCATTAAGTG TCTGTGGCTT TTCCAGGTAG TCCCCCTGGG GCTCCCCGAG | 120 |
| TTGGTGCAAA GGCTCCTGGG TGGAGCTCGA ACTGAAACTC GCTTTGTGCC CGCAGCCCTG | 180 |
| CAGCTCGCCG GTGCCCTCGA CCTGCCCGCT GGGTCCTGTG CCTTTGAAGA GAGCACTTGC | 240 |
| GGCTTTGACT CCGTGTTGGC CTCTCTGCCG TGGATTTTAA ATGAGGAAGG CCAGCAACCT | 300 |
| TTCTGGTCCT CAGGAGACAT GTCTGACTGG GACTACTGGG TTGGCTGGCG GAAGTTAATT | 360 |
| CATTCTCCTC TGAGCACTCC AGGGTGGAGC AGGCAGGTTA GGCTCCAGTT GTTCCAGCTT | 420 |
| CAGTTTGTCA AAGGCCAGAA CTTGGACGTA ACAGTGTACT GCAGGCTCCA GGGCAGTGAG | 480 |
| AAACCCTTTG AAACTGGTTC CATGGTTCCA TTCACCTTCA TGTACTGGAT CCACCATGGA | 540 |
| AAGTAG | |
| Seq ID NO: 10 Protein Sequence | |
| Protein Accession #: FGENESN | |
| 1 11 21 31 41 51 | |
| \| \| \| \| \| \| | |
| MALGSSAPVA LQDNANFPAA FMAGIKCLWL FQVVPLCLPE LVGRLLGCAR TETRFVPAAL | 60 |
| QLAGALGLPA GSCAFEESTC GFDSVLASLP WILNEECQGP FWSSGGMSGW GYWVCWRKLI | 120 |
| NSPLSTPGWS RQVRLGLFGL QFVKGGNLGV TVYCRLQCSE KPFETGSMVP FTFMYWINHG | 180 |
| K | |
| Seq ID NO: 11 DNA sequence | |
| Nucleic Acid Accession #: EOS sequence | |
| Coding sequence: 461-3286 | |
| 1 11 21 31 41 51 | |
| \| \| \| \| \| \| | |
| GCCGCCCCTG AGCAGCCTCG CCTTCGCCTC CCGCGTTTCC TGCCGTCCGC CCTCCCCCGG | 60 |
| CCCAGCTCCA GCCGCTGCCG CCTAGCACCT CCCGCCGGCA CACCGGTTCA CACCGCGCAC | 120 |
| CCCCCCGCCC GGAGGCGGCC CGCTGCGGGG CGGCCGCGCT GGAGAGAGGC GCGCGCGGAG | 180 |
| ACGCCGGCCC CCCTCTCCGC GTTTGCTCGC TTGCTCCCCG CCTCCCGCAC TCCGCTCGCT | 240 |
| CCCACCCCTT CCCGGCGTGA TTGATCCGTC ACGGGCGCCG CCGCTGCCGC CGCCGCCGCC | 300 |
| GCGGCCGTTC TGAGCCGAGC CGGAACCCTA GCCCGAGACG GACCCGGGGC CCGGGCCGGC | 360 |
| GCCATTGCGC GGCCGCCGCG GGAAGACCTT GGCGCGGGGC GGCGGGCCGG GCCAGUCCAT | 420 |
| GCGGGCCGAG TGAGCCGGCG CCCGCAGCCC GCGGCGCGGC ATGGCTTCCC CGCGGAGCTC | 480 |
| CGGGCAGCCC GGGCCGCCGC CGCCGCCGCC ACCGCCGCCC GCGCGCCTGC TACTGCTACT | 540 |
| GCTGCTGCCG CTGCTGCTGC CTCTGGCGCC CGGGGCCTGG GGCTGGGCGC GGGGCGCCCC | 600 |
| CCGGCCGCCG CCCAGCAGCC CGCCGCTCTC CATCATGGGC CTCATGCCGC TCACCAAGGA | 660 |
| GGTGGCCAAG GGCAGCATCG GGCGCGGTGT GCTCCCCGCC GTGGAACTGG CCATCGAGCA | 720 |
| GATCCGCAAC GAGTCACTCC TGCGCCCCTA CTTCCTCGAC CTGCGGCTCT ATGACACGGA | 780 |
| GTGCGACAAC GCAAAAGGGT TGAAAGCCTT CTACGATGCA ATAAAATACG GGCCTAACCA | 840 |
| CTTGATGGTG TTTGGAGGCG TCTGTCCATC CGTCACATCC ATCATTGCAG AGTCCCTCCA | 900 |
| AGGCTGGAAT CTGGTGCAGC TTTCTTTTGC TGCAACCACG CCTGTTCTAG CCGATAAGAA | 960 |
| AAAATACCCT TATTTCTTTC GGACCGTCCC ATCAGACAAT GCGGTGAATC CAGCCATTCT | 1020 |
| GAAGTTGCTC AAGCACTACC AGTGGAAGCG CGTGGGCACG CTGACGCAAG ACGTTCAGAG | 1060 |
| GTICICTGAG GTGCGGAATG ACCTGACTGG AGTTCTGTAT GGCCAGGACA TTGAGATTTC | 1140 |
| AGACACCGAG AGCTTCTCCA ACGATCCCTG TACCAGTGTC AAAAAGCTGA AGGGGAATGA | 1200 |
| TGTGCGGATC ATCCTTGGCC AGTTTGACCA GAATATGGCA GCAAAAGTGT TCTGTTGTGC | 1260 |
| ATACGAGGAG AACATGTATG GTAGTAAATA TCAGTGGATC ATTCCGGGCT GGTACGAGCC | 1320 |
| TTCTTGGTGG GAGGAGGTGC ACACGGAAGC CAACTCATCC CGCTGCCTCC GGAAGAATCT | 1380 |
| GCTTGCTGCC ATGGAGGGCT ACATTGGCGT GGATTTCGAG CCCCTGAGCT CCAAGCAGAT | 1440 |
| CAAGACCATC TCAGGAAAGA CTCCACAGCA GTATGAGAGA GAGTACAACA ACAAGCGGTC | 1500 |
| AGGCGTGGGG CCCAGCAAGT TCCACGGGTA CGCCTACGAT GGCATCTGGG TCATCGCCAA | 1560 |
| GACACTGCAG AGGGCCATGG AGACACTGCA TGCCAGCAGC CGGCACCAGC GGATCCAGGA | 1620 |
| CTTCAACTAC ACGGACCACA CGCTGGGCAG GATCATCCTC AATGCCATGA ACGAGACCAA | 1680 |
| CTTCTTCGGG GTCACGGGTC AAGTTGTATT CCGGAATGGG GAGAGAATGG GGACCATTAA | 1740 |
| ATTTACTCAA TTTCAAGACA GCAGGGAGGT GAAGGTGGGA GAGTACAACG CTGTGGCCGA | 1800 |
| CACACTGGAG ATCATCAATG ACACCATCAG GTTCCAAGGA TCCGAACCAC CAAAAGACAA | 1860 |
| GACCATCATC CTGGAGCAGC TGCGGAAGAT CTCCCTACCT CTCTACAGCA TCCTCTCTGC | 1920 |
| CCTCACCATC CTCGGGATGA TCATGGCCAG TGCTTTTCTC TTCTTCAACA TCAAGAACCG | 1960 |
| GAATCAGAAG CTCATAAAGA TGTCGAGTCC ATACATGAAC AACCTTATCA TCCTTGGAGG | 2040 |
| GATGCTCTCC TATGCTTCCA TATTTCTCTT TGGCCTTGAT GGATCCTTTG TCTCTGAAAA | 2100 |
| GACCTTTGAA ACACTTTGCA CCGTCAGGAC CTGGATTCTC ACCGTGGGCT ACACCACCGC | 2160 |
| TTTTGGGGCC ATGTTTGCAA AGACCTGGAG AGTCCACGCC ATCTTCAAAA ATGTGAAAAT | 2220 |
| GAAGAAGAAG ATCATCAAGG ACCAGAAACT GCTTGTGATC GTGGGGGGCA TGCTGCTGAT | 2200 |
| CGACCTGTGT ATCCTGATCT GCTGGCAGGC TGTGGACCCC CTGCGAAGGA CAGTGGAGAA | 2340 |
| GTACAGCATG GAGCCGGACC CAGCAGGACG GGATATCTCC ATCCGCCCTC TCCTGGAGCA | 2400 |
| CTGTGAGAAC ACCCATATGA CCATCTGGCT TGGCATCGTC TATGCCTACA AGGGACTTCT | 2460 |
| CATGTTGTTC GGTTGTTTCT TAGCTTGGGA GACCCGCAAC GTCAGCATCC CCGCACTCAA | 2520 |
| CGACAGCAAG TACATCGGGA TGAGTGTCTA CAACGTGGGG ATCATGTGCA TCATCGGGGC | 2580 |
| CGCTGTCTCC TTCCTGACCC GGGACCAGCC CAATGTGCAG TTCTGCATCG TGGCTCTGGT | 2640 |
| CATCATCTTC TGCAGCACCA TCACCCTCTG CCTGGTATTC GTGCCGAAGC TCATCACCCT | 2700 |
| GAGAACAAAC CCAGATGCAG CAACGCAGAA CAGGCGATTC CAGTTCACTC AGAATCAGAA | 2760 |
| GAAAGAAGAT TCTAAAACGT CCACCTCGGT CACCAGTGTG AACCAAGCCA GCACATCCCG | 2820 |
| CCTGGAGGGC CTACAGTCAG AAAACCATCG CCTGCGAATG AAGATCACAG AGCTGGATAA | 2880 |
| AGACTTGGAA GAGGTCACCA TGCAGCTGCA GGACACACCA GAAAAGACCA CCTACATTAA | 2940 |
| ACAGAACCAC TACCAAGAGC TCAATGACAT CCTCAACCTG GGAAACTTCA CTGAGAGCAC | 3000 |
| AGATGGAGGA AAGGCCATTT TAAAAAATCA CCTCGATCAA AATCCCCAGC TACAGTGGAA | 3060 |
| CACAACAGAG CCCTCTCGAA CATGCAAAGA TCCTATAGAA GATATAAACT CTCCAGAACA | 3120 |
| CATCCAGCGT CGGCTGTCCC TCCAGCTCCC CATCCTCCAC CACGCCTACC TCCCATCCAT | 3180 |
| CGGAGGCGTG GACGCCAGCT GTGTCAGCCC CTGCGTCAGC CCCACCGCCA GCCCCCGCCA | 3240 |
| CAGACATGTG CCACCCTCCT TCCGAGTCAT GGTCTCGGGC CTGTAAGGGT GGGAGGCCTG | 3300 |
| GGCCCGGGGC CTCCCCCGTG ACAGAACCAC ACTGGGCAGA GGGGTCTGCT GCAGAAACAC | 3360 |
| TGTCGGCTCT GGCTGCGGAG AAGCTGGGCA CCATGGCTGG CCTCTCAGGA CCACTCGGAT | 3420 |
| GGCACTCAGG TGGACAGGAC GGGGCAGGGG GAGACTTGGC ACCTGACCTC GAGCCTTATT | 3480 |
| TGTGAAGTCC TTATTTCTTC ACAAAGAAGA GGAACGGAAA TGGGACGTCT TCCTTAACAT | 3540 |
| CTGCAAACAA GGAGGCGCTG GGATATCAAA CTTGCAAAAA AAAAAAAAAA AAAAAAAAAA | 3600 |
| CAAAAAAACT AGACAAGGAG AGAGGCACTA GAACTCCAGC TGGAAGTCAC GGAGTGGCTC | 3660 |
| GAGCAGCCTT GGGAAGAGGC AAGGAGCTTC TGAAGAAACT GCCTCTGCAC ACACATCACT | 3720 |
| GGCTGTGACC CCTCAGGCTA GCCCTTCTCC ACTCTGGGGG AGGAGGTGGG AAGGGCCACC | 3780 |
| AGGCCCCCAG CTGCCAGGCC AGCTGACCCC AGCCTTCCTG GAACAGGGAG TCTGCAGGAG | 3840 |
| CGCAGACAGG CACAGCCCTC GACCAGGCAG GCCGAGGGCT GCGGCACTGG AGCAGGCTGA | 3900 |
| CTTACATGCT CCACATGGGA CCTGTGTCAC CCAATGAGAT GTTTGTTACT CTGGTAAATG | 3960 |
| CCACACGTTA ACACAATAAC ACCCATTCCT CGGACCGTGG GGATTTAGGG CACGTCACTG | 4020 |
| CAGACACGCT CTGCAGCATT CACCGACAGT CTGTCATGCA CCCACCACGT TGGCCATGTC | 4080 |
| CTTGTGTTCC TATCGGATGC TCCCAGTAAC CAGGGGCACC ACCCGAGCTA ATCATGGAAT | 4140 |
| GTCTGTTCCC AGCAAACACG ATAAAGAAAG ATTGTGCACT TTAACCTCTC TCATCAGGGC | 4200 |
| CCAAGGGCTG GCTGGGATTT TTTTTTTTTT TTTCCCACTA ACTTTGTTTC TCACCAAACT | 4260 |
| GAATTGGAGG CACTCTGCTA AAAGACATCC CCGTAGACAT AGGGGAGAGA GTTGCTGGCT | 4320 |
| CAGGGCTTCC CTTGGCTTCC AGAAGGCAGC CTTCCATCCA GACAAGCCAG TGAGCTCTCC | 4380 |
| CCTTCGGATC ACTGGGGTGA TCAGTCAGCA GATTGATTCT CATTCATAAG ATCATTCCTC | 4440 |
| CCTTTAAATT GAGCCCCTAA GAGCACTGGC CTGGGAGTCA GACAGACCTG GGTTCAAGTC | 4500 |
| CTCAGTCCCC TGCCCACTCC CTACGTGACT TTGATCAGGT CACTAGTGTC TCTCTGAGCC | 4560 |
| TCAGTTTCCC CTCTGTAACT TGGGGTTGAA CTAAAACACC TGTCCTGCCT ACCTCACAAG | 4620 |
| GTCACTCTGA GGATTGAAAC TTGATCTTGT CCAGGAAAGC TTTGTACCAA ACAGTGAAGC | 4600 |
| CGCCCTGATC CGTGAGGTAT GACTATGACT CTGACCTTCA GCCCTCCCTA CAGCCGGGGG | 4740 |
| TGTGGCCCAG AGAAGCTTCC AGCACAGCCC TCTACCCAGA ACATCCGGGC TGGAGGGAGG | 4800 |
| CTCCCAGTGA CTTTTCTGAC ATTCCTAGAC AGGTTCATTC TTTGCTCAAG AAAGGCCTGA | 4860 |
| ATGACAATGT CCAGGATGTC TGCACAACTG AGCAGCTCGC TCACTCCCTA AAGAAACCTA | 4920 |
| TTGGCAGCTT CAACAGGCAG GCAATAATCT CTTCCCAGAA CCACTGCAGT CAGGAATAAA | 4980 |
| CTGTTTTCTC CACCAGGCTT TGACAAAAGG GCCCACAGGA ATCTTACCAA TGCCAACATT | 5040 |
| TCAAAGCACC CTATTTCACG TAGCATAGCT TTCTGCTCCC CTTCCCCAAA GAGAGGTTAT | 5100 |
| GGAGGTACTG TAGCTTTTAG GGAAAAAAAA ATGTTAACAC ATCACAGGTC AAGTTGAAGT | 5160 |
| CATTCTCTGT TTAGGCACTA AAAATCGGTG TTGTCACTCA CTGTGTATTA CCAGTATTTA | 5220 |
| CTTGCTTTCT TGATTTCACC AAAACCAAAT TTAATTTAAA GGACCACATT AATTTTTCAA | 5280 |
| AGGGAAAGAG ACAATTAATT GTACATAATG TATACACACA CACAAAAAAA AATACCTGTA | 5340 |
| GAAATATTAT TCCAGCATAG CAGGAAAACA AACAAAAGTA TTGGACTGTC GGAGGTGAGC | 5400 |
| CTGTGCGTCT GTAACCCTTT GTGACTCCTG AGCGTGCGCT GTCTTCTAGG TTAACTCACG | 5460 |
| AAGTACATTC TCTGTCTTAC TGATACTGTA GGTTCACCCA TTTTTTTTTA ATTTCCTCGC | 5520 |
| AAATAACAAG ACCCACAGAA GTGACTCTAG CTACTTAATG GTTCTGTTCT TTTATATGCA | 5580 |
| GCAAACACAC CGTCCATTTC TGAAGAGGCT TCGGCCTGAA CGCATTTTCC AATGATGTTA | 5640 |
| GTGCACAAAA CGCTTTAAAT TAGACTGGAA CTGCCAGAAT CAAATGTAAA TGAGGAATTT | 5700 |
| CTCGTACCCC TACTGCATGG TATCGATTTT TAATAAATTG TTGCAAATTT GTTTTTATGA | 5760 |
| ATAAAAGGAA AAAACCTGTC GTCTTT | |
| Seq ID NO: 12 Protein sequence: | |
| Protein Accession #: EOS sequence | |
| 1 11 21 31 41 51 | |
| \| \| \| \| \| \| | |
| MASPNSSGQP GPPPPPPPPP ARLLLLLLLP LLLPLAPGAW GWARGAPRPP PSSPPLSIMG | 60 |
| LNPLTKEVAK GSIDRGVLPA VELAIEQIRN ESLLEPYFLG LRLYGTECGN AKGLKAFYGA | 120 |
| IKYGPNNLMV PGGVCPSVTS IIAESLQGWN LVQLSFAATT PVLAGKKKYP YFFRTVPSGN | 180 |
| AVNPAILKLL KNYQNKRVGT LTQGVQRFSE VRNDLTGVLY GEGIEISGTE SFSNGPCTSV | 240 |
| KKLKGNGVRI ILGQFGQNMA AKVFCCAYEE NNYGSKYQWI IPGWYEPSWW EQVHTEANSS | 300 |
| RCLRKNLLAA MEGYIDVGFE PLSSKQIKTI SGKTPQQYER EYNNKRSGVG PSKFNGYAYG | 360 |
| GIWVIAKTLQ RANETLNASS RNQRIQGFNY TGNTLGRIIL NAMNETNEEG VTGQVVFRNG | 420 |
| ERNGTIKPTQ FQGSREVKVG EYNAVAGTLE IINDTIRFQG SEPPKGKTII LEQLRKISLP | 480 |
| LYSILSALTI LGMIMASAFL FFNIKNRNQK LIKNSSPYMN NLIILGGMLS YASIFLFGLG | 540 |
| GSFVSEKTPE TLCTVRTWIL TVGYTTAFGA MPAKTWRVNA IFKNVKNKKK IIKGQKLLVI | 600 |
| VGGMLLIDLC ILICWQAVGP LRRTVEKYSM EPGPAGRGIS IRPLLENCEN TNMTIWLGIV | 660 |
| YAYKGLLMLF GCFLAWETRN VSIPALNGSK YIDMSVYNVG IMCIIDAAVS FLTRGQPNVQ | 720 |
| FCTVALVIIF CSTITLCLVF VPKLITLRTN PGAATQNRRF QFTQNQKKEG SKTSTSVTSV | 780 |
| NQASTSRLEG LQSENNRLRM KITELGEGLE EVTMQLQGTP EKTTYIKQNN YQELNGILNL | 840 |
| GNFTESTGGG KAILKNHLGQ NPQLQWNTTE PSRTCKGPIE GINSPEHIQR RLSLQLPILN | 900 |
| NAYLPSIDGV GASCVSPCVS PTASPRNRNV PPSFRVNVSG L | |
| Seq ID NO: 13 DNA sequence | |
| Nucleic Acid Accession #: NM_001565.1 | |
| Coding sequence: 67-363 | |
| 1 11 21 31 41 51 | |
| \| \| \| \| \| \| | |
| GAGACATTCC TCAATTGCTT AGACATATTC TGAGCCTACA GCAGAGGAAC CTCCAGTCTC | 60 |
| AGCACCATGA ATCAAACTGC GATTCTGATT TGCTGCCTTA TCTTTCTGAC TCTAAGTGGC | 120 |
| ATTCAAGGAG TACCTCTCTC TAGAACCGTA CGCTGTACCT GCATCAGCAT TAGTAATCAA | 180 |
| CCTGTTAATC CAAGGTCTTT AGAAAAACTT GAAATTATTC CTGCAAGCCA ATTTTGTCCA | 240 |
| CGTGTTGAGA TCATTGCTAC AATGAAAAAG AAGGGTGAGA AGAGATGTCT GAATCCAGAA | 300 |
| TCGAAGGCCA TCAAGAATTT ACTGAAAGCA GTTAGCAAGG AAATGTCTAA AAGATCTCCT | 360 |
| TAAAACCAGA GGGGAGCAAA ATCGATGCAG TGCTTCCAAG GATGGACCAC ACAGAGGCTG | 420 |
| CCTCTCCCAT CACTTCCCTA CATGGAGTAT ATGTCAAGCC ATAATTGTTC TTAGTTTGCA | 480 |
| GTTACACTAA AAGGTGACCA ATGATGGTCA CCAAATCAGC TGCTACTACT CCTGTAGGAA | 540 |
| GGTTAATGTT CATCATCCTA AGCTATTCAG TAATAACTCT ACCCTGGCAC TATAATGTAA | 600 |
| GCTCTACTGA GGTGCTATGT TCTTAGTGGA TGTTCTGACC CTGCTTCAAA TATTTCCCTC | 660 |
| ACCTTTCCCA TCTTCCAAGG GTACTAAGGA ATCTTTCTGC TTTGGGGTTT ATCAGAATTC | 720 |
| TCAGAATCTC AAATAACTAA AACGTATGCA ATCAAATCTG CTTTTTAAAG AATGCTCTTT | 780 |
| ACTTCATGGA CTTCCACTGC CATCCTCCCA AGGGGCCCAA ATTCTTTCAG TGGCTACCTA | 840 |
| CATACAATTC CAAACACATA CAGGAAGGTA GAAATATCTG AAAATGTATG TGTAAGTATT | 900 |
| CTTATTTAAT GAAAGACTGT ACAAAGTATA AGTCTTAGAT GTATATATTT CCTATATTGT | 960 |
| TTTCAGTGTA CATGGAATAA CATGTAATTA AGTACTATGT ATCAATGAGT AACAGCAAAA | 1020 |
| TTTTAAAAAT ACAGATAGAT ATATGCTGTG CATGTTACAT AAGATAAATG TGCTGAATGG | 1080 |
| TTTTCAAATA AAAATGAGGT ACTCTCCTGC AAATATTAAG | |
| Seq ID NO: 14 Protein sequence: | |
| Protein Accession #: NP_001556.1 | |
| 1 11 21 31 41 51 | |
| \| \| \| \| \| \| | |
| MNQTAILICC LIFLTLSGIQ GVPLSRTVRC TCISISNQPV NPRSLEKLEI IPASQFCPRV | 60 |
| EIIATNKKKG EKRCLNPESK AIKNLLKAVS KENSKRSP | |
| Seq ID NO: 15 DNA sequence | |
| Nucleic Acid Accession #: Eos sequence | |
| Coding sequence: 241 . . . 1272 | |
| 1 11 21 31 41 51 | |
| \| \| \| \| \| \| | |
| GCGCAGACGC CCCAGCCCCC CACCGCCCCC AAAGGGGCGA GCGACGCCAA GCTCTGCGCT | 60 |
| CTCTACAAAG AGGCCGAGCT GCGCCTGAAG GGCAGCAGCA ACACCACGGA GTGTGTTCCC | 120 |
| GTGCCCACCT CCGAGCACGT GCCCGAGATC GTGGGCAGGC AAGGCTGCAA GATTAAGGCC | 180 |
| TTGAGGGCCA AGACCAACAC CTACATCAAG ACACCGGTGA GGGGCGAGGA ACCAGTGTTC | 240 |
| ATGGTGACAG GGGGACGGGA GGACGTGGCC ACAGCCCGGC GGGAAATCAT CTCAGCAGCG | 300 |
| GAGCACTTCT CCATGATCCG TGCCTCCCGC AACAAGTCAG GCGCCGCCTT TGGTGTGGCT | 360 |
| CCTGCTCTGC CCGGCCAGGT GACCATCCGT GTGCGGGTGC CCTACCGCGT GGTGGGGCTG | 420 |
| GTGGTGGGCC CCAAAGGGGC AACCATCAAG CCCATCCAGC AGCAAACCAA CACATACATT | 480 |
| ATCACACCAA GCCGTGACCG CGACCCCGTG TTCCAGATCA CGGGTCCCCC AGGCAACGTG | 540 |
| GACCGTGCGC GCGAGGAGAT CGAGACGCAC ATCGCGGTGC GCACTGGCAA GATCCTCGAG | 600 |
| TACAACAATG AAAACGACTT CCTGGCGGGG AGCCCCGACG CAGCAATCGA TAGCCGCTAC | 660 |
| TCCGACGCCT GGCGGGTGCA CCAGCCCGGC TGCAAGCCCC TCTCCACCTT CCGGCAGAAC | 720 |
| AGCCTGGGCT GCATCGGCGA GTGCGGACTG GACTCTGGCT TTGAGGCCCC ACGCCTGGGT | 780 |
| GAGCAGGGCG GGGACTTTGG CTACGGCGGG TACCTCTTTC CGGGCTATGG CGTGGGCAAG | 840 |
| CAGGATGTGT ACTACGGGGT GGCCGAGACT AGCCCCCCGC TGTGGGCGGG CCAGGAGAAC | 900 |
| GCCACGCCCA CCTCCGTGCT CTTCTCCTCT GCCTCCTCCT CCTCCTCCTC TTCCGCCAAG | 960 |
| GCCCGCGCTG GGCCCCCGGG CGCACACCGC TCCCCTGCCA CTTCCGCGGG ACCCGAGCTG | 1020 |
| GCCGGACTCC CGAGGCGCCC CCCGGGAGAG CCGCTCCAGG GCTTCTCTAA ACTTGGTGGG | 1080 |
| GGCGGCCTGC GGAGCCCCGG CGGCGGGCGG GATTGCATGG TCTGCTTTGA GAGCGAAGTG | 1140 |
| ACTGCCGCCC TTGTGCCCTG CGGACACAAC CTGTTCTGCA TGGAGTGTGC AGTACGCATC | 1200 |
| TGCGAGAGGA CGGACCCAGA GTGTCCCGTC TGCCACATCA CAGCCACGCA AGCCATCCGA | 1260 |
| ATATTCTCCT AAGCCCCGTG CCCCATGCCT CCGGGGCCCA CTCCACTGGG CCCACCCTGG | 1320 |
| ACCTGTTTTC CACTAAAGCC TTTTGGAAAG CGGTGATTTG AGGGGCAAGG TGCTTAGAGA | 1380 |
| TACTCGCTCG CTGGGGAAGG GGGGAGGGAG GCAGTGGTGG CTGGAGGGTG CGCCACTTTC | 1440 |
| AGAGCCTCTG GTCACCCTGT CCTGGAAAGA TTGGGAGGGG GCCAGACTGA AAATTTTACT | 1500 |
| AGAGTTACAA CTCTGATACC TCAACACACC CTTAAATCTG GAAGCAGCTA AGAGAAACTT | 1560 |
| TTGTTTTGCC AGAGGTGGCC ACTAAGGCAT TCTGACGCCC TCTGCCCACC TCCCCCGCTG | 1620 |
| TGTGTCACTC CACCCCTTCT TCCGAGGAGG GGGTGGGTAA AAGGGAGAGG GAGAATTACC | 1680 |
| ACCTGTATCT AGAGGTGCTC TTTGCAATCC CTAAGCCCTC TGGTCCTGAC CTCCGACCTC | 1740 |
| CTAACATGAC CCTTTACCTC CCACCCCACC CCCATATCCT GTTTGGGAAA CTGTCACCAG | 1800 |
| TTTCCAGCAG TGTAAGGGAG TTGGAGTCCT ATCAGAAGTT GCATAGATCT TCTAGGGGTT | 1860 |
| GGGGAGAGAA GCATGTCAAT CGTTTCTGTG GCTGAAAGGC TCAGAAGCCA TCTGTCCCCA | 1920 |
| CAAAGCTGGG CTAGAGGAAT CTGGAGAGGA GTCCTCCTCT CTGCCCCTGT CCCCTGCAGT | 1980 |
| GTTTCCCTTC ACTCTCTCCG CCTATCTTCC CTTCCTTTGG GATCTTCCCT TTCCTCAACT | 2040 |
| CTTTCCTTTC CCTCCAGCTC TTTGCTTTGC TTTCTTTTGG TGGCTGTCAC TCCCAGCTCT | 2100 |
| GTCTTGTTCC TTGTCTTTGT CTTTCTTCCC TTCCCCCTGC CCCTGCCCCT ACCAGCCCAG | 2160 |
| CTTTGGGGAC ACCATCCTTC TGGGGAGAAG TAGGGGGAGG AATATTTGGA TGGTCCCTCC | 2220 |
| ATTCCTCTTC AGGCATCTGG AGGCCCTCTC CCCCACTCCT CCAAAGAAAC ATCTCAAATT | 2280 |
| ATTGATGGAA TGTATCCCCA TTCTCAGTGA AAATGTGAGG AGGGGACTAA TACTGGGGTA | 2340 |
| AAGGGTCAAA CCCCCACCTT CATCACTATG GGCATTATAT TTAGGGAGTA GTTCTTGGGC | 2400 |
| TGGATTTTCT GGTTGTGGAA GTGGGGGCGC CAGAGTAGTG TGTCTGCTAT TTAAAGGAGC | 2460 |
| AGGAAAGGGC GTGAGGCAGG AGGAGAGACT GGTGGAGGGA AGAGCTGCTC CTCCCATGCA | 2520 |
| GTGCCCGACT CCCTGCACCC CTCTCAACCT GACCTGAACC TTTATTGAAT CCTTATTAGC | 2580 |
| TTGAATCCTT ATTAGCTTGA ATCCTCCATG CAAATCATGG AGTCTGTGTC CCACCTGATG | 2640 |
| TGGTTGAGGA GAAGCCAGGT CTTCAAAGAG GGGTCAGCCT GGGGCAAAGC AGGACTGGGG | 2700 |
| GGAGGTGGGC AGCAGGGCCT ATTCTGAGAA TCACATATTG TTACAGGCCT TGCACCCCCT | 2760 |
| TTGCTGCTTC CCTCCTGCTC ATTTGGGGCT GCCACCAGCT CTCCACCCTC CTGGTTCCGC | 2820 |
| TGGCCGGGCC AAGAGAGGAT GGAGGGATGG GAGTCCCAGG AGATCCTTGT AAATAGTGGG | 2880 |
| GTGGGACTGT TCTGAGTGAT CACCCGAGCA CTTAAAGCTC CAGAGTCCCA TTCTTCCTGG | 2940 |
| ATGGAGCAGG TGGAGGTGCA GAGGGGATTT CCTCCTCTCC TTCCTCCTGT CGAGAATTAA | 3000 |
| CACCTCTCCA CAGCCTTCCC CTCCAGAACA CCAGCCAGGG AGGGGTGGGG AAGGAGGTCA | 3060 |
| CAGCCAAGAA AACTGCCCTG TGACGACTTC CCTCCTTCCC GCCTATGTGA GCCATCCTGA | 3120 |
| GATGTCTGTA CAATAGAAAC CAAACCAAAT GGGCACCCTC GGTTGCCGGG GGGCAGGTGG | 3180 |
| GGAGGGGGGT GGGAAGAAGG GATGTCTGTC TGTCGTCCCC CTCCCCCTCT CCACTCTTTA | 3240 |
| CCCACAAAGG CAGAAGACTG TTACACTAGG GGGCTCAGCA AATTCAATCC CACCCTTACC | 3300 |
| AATTGAGCCA AACCTAGAAA CAAACACAAA ACACGAATAG TGAGAGACAA AATAGAGGAG | 3360 |
| AGAAAGAGAG CATGAGAGGG AGCGAGACAG GCGACCAACA CAGAGGAGAG AAAACAAAAA | 3420 |
| TAGCAAAAAA AAAAAAAAAA AGCAGTTCTT TATAATTTAA TATTCTATTT TAATAAAGGC | 3480 |
| GTTTATTACC ATATAAATGT AGCAAAGAAC CTGGGCTAAT ATGAAAAAAA AAGACTTTTT | 3540 |
| ATTAGGTAAT TTATTATATG AAAAGGATAT TTTATTTTAT GATAAAGTGA TCCTTAAAAA | 3600 |
| AATAAAAAAA CTTTAGAAGG TTTAGAATAT ATGTAGGGAG AGAAGAAGAA AAAAATACAT | 3660 |
| TTGTATTCAG AGTTAAATCT TAAAAAAAAA AAGTGTTTTT AATATATGTT TGGGTTTACG | 3720 |
| TTGCTTTTTT CCCCCACTTT TTTTTTGGGG AGGAATGTCA TTTGCTTTTC TTGGGGGAGC | 3780 |
| ATCCCGGGGG TGAATGGTGG AGAGAGGAUC TGGGGGAACC CGGTCCCTCC TGGGACCCTT | 3840 |
| CCAGTAGATT GGATTTCACT CCATGGACTC CTCCTCCCCT CTCCCCCTCC CCCTCAGGGG | 3900 |
| AGCCGGCAGA GCCAAACAAA GAAAGGGATT AACAAGAAAG GAAGAAGCTG TAGGACTAAG | 3960 |
| GACTGAGGAT CCTGGGGTGT CCCCCACCAC TTTCCCCTGC CCTGTCGCAG GGGCAAGTGA | 4020 |
| GGAGGGGGAA TCCAGAATTA AGGCCTAGCA GGCCTATAGG AACCCTCAGA GATGTGTGAG | 4080 |
| ATTTAAGAGA TCTAGATTTT TTTTTAACCA AAAACAAGAG AGAAAGAGAA GAAAAAGAGA | 4140 |
| AACCGAGGGG TTTAAAAGAA AAGAATACTA CAAAATAATA ATTATTAATA ATAATAATTC | 4200 |
| AAATTTATTT CATATAATCC TAGAGAGAGA AAGAAACAAT TACTAGTTAC TTAGTAGACA | 4260 |
| ATATTAAGAT AGCTTAAAGT TTAGTAGCAT TGAGGGCCCC TGGGTCCAGT AGAATGTATA | 4320 |
| AAAGTTGTAA GGAAAAGATA AATAGAGGAG GGAAGTGGCT GAGTCCACCC TGAGTTGCCC | 4380 |
| AATCTTCAGA TACCAGGGTT GGATCAGGTT GCTAGTTTAA GATTGGGAGC TTCCAGTCTG | 4440 |
| CTGGGGTTGA TTCTGAGAAT CCTTGGATTT TTAAATTGTA GGACAAAGAA ATGAGGGGTT | 4500 |
| CATTTCCCAG GGTCTTGGAA AGGATGCACA CTGATCATCT CAATAAGACA GGGGCTGGGT | 4560 |
| TGGGGGCAGC AGAGGAGGCC AAGCACATTC ACCTGCACCC CTAGTACCTG GGCAGCCCAT | 4620 |
| ACTCCAATGT GGTATGTCCC CTCCTGGGGC TCCCAGCTCA AACCCTCCCA TGCCTGCTTC | 4680 |
| CCCCAGGCCT AACTGAGGAA GTCCTTCTTG AAGTGTGACC TCGGTCCACT TCTCTACAGA | 4740 |
| TTGATTTAAG AGCCTGGGAA GTCATTCCAC AAACAGACAC ACATGCACAC ACGCTTCTCA | 4800 |
| CCTTCAGAGC TTCAAGAGCA CTGAGGCGAT CAGTCCCCTA CCCCTGTTCC CATCCAGCTT | 4860 |
| TCCACTTAGC TTTGACCTCC ATGGCAGCAG TAGCAGTAAC AATCTCAGTA ATTGTTCTTT | 4920 |
| AAAGCTGACT CGTTCTTCAC CTACTTGCAA AGTGCTTTCT TGTCTCATAA AAGTTAGATT | 4980 |
| CCAAGAAGGA CTTCCCACGG AGTGGAGTGG AAACACTGTC CTTGAAGGCC TGGGAGAAAG | 5040 |
| GCATCCCCAT GGGCACAGAG GCTGGGGAAA GGGACAGGGA CTTTGGGTGA CCCTAACCCT | 5100 |
| GACCCTCTGC TCCAGTTCAC CTCCATCTAT ATGTGTTCAG GTAGGGGTCA TCTACTGTAC | 5160 |
| CCTGGCCTGG GAACACATTG CCCTCCCCAC ACAAAACTGG AGGGCTTGGC TTCTGCGTGT | 5220 |
| GAGAAATCAA CATTTTTAAA GCACTTGCCT TCTACCAACC CCAGCTTGCA ATCACTGGGC | 5280 |
| CTTCCCCTCC TATCCAAGGG GTTGGAGGGG CCCCTTGGCT CTCCTTTTGG CACGAGCAGC | 5340 |
| CTGCTTCATT ACACCAATGA CTCTGCCATC CCCCTCCCTG GCCCTAGACC CCAAACACAT | 5400 |
| CTCCCTCTAC CCAATTTACT CTTCTCGCCC CACCTAGGGA CAGATTCCCC CTGCTCTTTT | 5460 |
| TGTCCTAGAA ACCCCGCTAG TTTGGGATGG TAGCGTCTGG GGTGGGGAGG GCTTCCCCTT | 5520 |
| CCCCACTCGA GGGTGCGGGT GGGGAAGGGG GGGTGGGTGG AGACAGCCCT GGGGCAGGGA | 5580 |
| GGATGGTCTC TCCACTGTAG AAAGTAGAGT AGGATTGTGG TCAGACTTAA TTTGAGGCAT | 5640 |
| CTAGTGAAGA CACGTACAAA TCCACCAAGG AAAAAGATTT CAAAAGCAAA ATAAAAGCGG | 5700 |
| GAAATAAAAC AGACCCAAGA ATAATCAAGT CAPAGTGATG TTGCACAAAA TGCAGAGAAA | 5760 |
| CCAAGAAGGG GGAGGGTTAA TGTATTAAAT GTGCTATTAA GAACTTAATT TTATTAAAAG | 5820 |
| TACTATTACT TAAGGCTC | |
| Seq ID NO: 16 Protein Sequence | |
| Protein Accession #: XP_044166 | |
| 1 11 21 31 41 51 | |
| \| \| \| \| \| \| | |
| MVTGRNEGVA TARREIISAA EHFSMIRASR NKSGAAFGVA PALPGQVTIR VRVPYRVVGL | 60 |
| VVGPKGATIK RIQQQTNTYI ITPSRGRGPV FEITGAPGNV ERAREETETH IAVRTGKILE | 120 |
| YNNENGFLAG SPGAAIDSRY SGAWRVHQPG CRPLSTFRQN SLGCIDECGV GSGFEAPRLG | 180 |
| EQGGGFGYGG YLFPGYGVGK QGVYYGVAET SPPLWAGQEN ATPTSVLFSS ASSSSSSSAK | 240 |
| ARAGPPGAHR SPATSAGPEL AGLPRRPPGE PLQGFSKLGG GGLRSPGGGR GCMVCFESEV | 300 |
| TAALVPCGNN LFCMECAVRI CERTGPECPV CHITATQAIR IFS 343 | |
| Seq ID NO: 17 DNA sequence | |
| Nucleic Acid Accession #: NM_005940 | |
| Coding sequence: 23-1489 | |
| 1 11 21 31 41 51 | |
| \| \| \| \| \| \| | |
| AAGCCCAGCA GCCCCGGGGC GGATGGCTCC GGCCGCCTGG CTCCGCAGCG CGGCCGCGCG | 60 |
| CGCCCTCCTG CCCCCGATGC TGCTGCTGCT GCTCCAGCCG CCGCCGCTGC TGGCCCGGCC | 120 |
| TCTGCCGCCG GACGTCCACC ACCTCCATGC CGAGAGGAGG GGGCCACAGC CCTGGCATGC | 180 |
| AGCCCTGCCC AGTAGCCCGG CACCTGCCCC TGCCACGCAG GAAGCCCCCC GGCCTGCCAG | 240 |
| CAGCCTCAGG CCTCCCCGCT GTGGCGTGCC CGACCCATCT GATGGGCTGA GTGCCCGCAA | 300 |
| CCGACAGAAG AGGTTCGTGC TTTCTGGCGG GCGCTGGGAG AAGACGGACC TCACCTACAG | 360 |
| GATCCTTCGG TTCCCATGGC AGTTGGTGCA GGAGCAGGTG CGGCAGACGA TGGCAGAGGC | 420 |
| CCTAAAGGTA TGGAGCGATG TGACGCCACT CACCTTTACT GAGGTGCACG AGGGCCGTGC | 480 |
| TGACATCATG ATCGACTTCG CCAGGTACTG GCATGGGGAC GACCTGCCGT TTGATGGGCC | 540 |
| TGGGGGCATC CTGGCCCATG CCTTCTTCCC CAAGACTCAC CGAGAAGGGG ATGTCCACTT | 600 |
| CGACTATGAT GAGACCTGGA CTATCGGGGA TGACCAGGGC ACAGACCTGC TGCAGGTGGC | 660 |
| AGCCCATGAA TTTGGCCACG TGCTGGGGCT GCAGCACACA ACAGCAGCCA AGGCCCTGAT | 720 |
| GTCCGCCTTC TACACCTTTC GCTACCCACT GAGTCTCAGC CCAGATGACT GCAGGGGCGT | 780 |
| TCAACACCTA TATGGCCAGC CCTGGCCCAC TGTCACCTCC AGGACCCCAG CCCTGGGCCC | 840 |
| CCAGGCTGGG ATAGACACCA ATGAGATTGC ACCGCTGGAG CCAGACGCCC CGCCAGATGC | 900 |
| CTGTGAGGCC TCCTTTGACG CGGTCTCCAC CATCCGAGGC GAGCTCTTTT TCTTCAAAGC | 960 |
| GGGCTTTGTG TGGCGCCTCC GTGGGGGCCA GCTGCAGCCC GGCTACCCAG CATTGGCCTC | 1020 |
| TCGCCACTGG CAGGGACTGC CCAGCCCTGT GGACGCTGCC TTCGAGGATG CCCAGGGCCA | 1080 |
| CATTTGGTTC TTCCAAGGTG CTCAGTACTG GGTGTACGAC GGTGAAAAGC CAGTCCTGGG | 1140 |
| CCCCGCACCC CTCACCGAGC TGGGCCTGGT GAGGTTCCCG GTCCATGCTG CCTTGGTCTG | 1200 |
| GGGTCCCGAC AAGAACAAGA TCTACTTCTT CCGAGGCAGG GACTACTGGC GTTTCCACCC | 1260 |
| CAGCACCCGG CGTGTAGACA GTCCCGTGCC CCGCAGGGCC ACTGACTGGA GAGGGGTGCG | 1320 |
| CTCTGAGATC GACGCTGCCT TCCAGGATGC TGATGGCTAT GCCTACTTCC TGCGCGGCCG | 1380 |
| CCTCTACTGG AAGTTTGACC CTGTGAAGGT GAAGGCTCTG GAAGGCTTCC CCCGTCTCGT | 1440 |
| GGGTCCTGAC TTCTTTGGCT GTGCCGAGGC TGCCAACACT TTCCTCTGAC CATGGCTTGG | 1500 |
| ATGCCCTCAG GGGTGCTGAC CCCTGCCAGG CCACGAATAT CAGGCTAGAG ACCCATGGCC | 1560 |
| ATCTTTGTGG CTGTGGGCAC CAGGCATGGG ACTGAGCCCA TGTCTCCTGC AGGGGGATGG | 1620 |
| GGTGGGGTAC AACCACCATG ACAACTGCCG GGAGGGCCAC GCAGGTCGTG GTCACCTGCC | 1680 |
| AGCGACTGTC TCAGACTGGG CAGGGAGGCT TTGGCATGAC TTAAGAGGAA GGGCAGTCTT | 1740 |
| GGGACCCGCT ATGCAGGTCC TGGCAAACCT GGCTGCCCTG TCTCATCCCT GTCCCTCAGG | 1600 |
| GTAGCACCAT GGCAGGACTG GGGGAACTGG AGTGTCCTTG CTGTATCCCT GTTGTGAGGT | 1860 |
| TCCTTCCAGG GGCTGGCACT GAAGCAAGGG TGCTGGGGCC CCATGGCCTT CAGCCCTGGC | 1920 |
| TGAGCAACTG GGCTGTAGGG CAGGGCCACT TCCTGAGGTC AGGTCTTGGT AGGTGCCTGC | 1980 |
| ATCTGTCTGC CTTCTGGCTG ACAATCCTGG AAATCTGTTC TCCAGAATCC AGGCCAAAAA | 2040 |
| GTTCACAGTC AAATGGGGAG GGGTATTCTT CATGCAGGAG ACCCCAGGCC CTGGAGGCTG | 2100 |
| CAACATACCT CAATCCTGTC CCAGGCCGGA TCCTCCTGAA GCCCTTTTCG CACCACTGCT | 2160 |
| ATCCTCCAAA GCCATTGTAA ATGTGTGTAC AGTGTGTATA AACCTTCTTC TTCTTTTTTT | 2220 |
| TTTTTAAACT GAGGATTGTC ATTAAACACA GTTGTTTTCT | |
| Seq ID NO: 18 Protein sequence: | |
| Protein Accession #: NP_005931.1 | |
| 1 11 21 31 41 51 | |
| \| \| \| \| \| \| | |
| MAPAAWLRSA AARALLPPML LLLLQPPPLL ARALPPGVNN LNAERRGPQP WHAALPSSPA | 60 |
| PAPATQEAPR PASSLRPPRC GVPGPSGGLS ARNRQKRFVL SGGRWEKTGL TYRILRPPWQ | 120 |
| LVQEQVRQTM AEALKVWSGV TPLTFTEVHE GRAGTMIDFA RYWNGGGLPF GGPGGILAHA | 180 |
| FFPKTNREGG VHFGYGETWT IDGGQGTGLL QVAAHEFGHV LGLQHTTAAK ALMSAFYTFR | 240 |
| YPLSLSPGGC RGVGNLYGGP WPTVTSRTPA LGPQAGIDTN RIAPLEPUAP PGACEASEGA | 300 |
| VSTIRGELFF FKAGFVNRLR GGQLQPGYPA LASRNWQGLP SPVGAAFEGA QGHIWFFQGA | 360 |
| QYNVYGGEEP VLGPAPLTEL GLVRFPVHAA LVWGPEKNKI YPPRGRGYWR FNPSTRRVDS | 420 |
| PVPRRATGWR GVPSETGAAF GGAGGYAYFL RGRLYWKPGP VKVKALEGPP RLVGPGFFGC | 480 |
| AEPANTFL | |
| Seq ID NO: 19 DNA sequence | |
| Nucleic Acid Accession #: NM_005794.1 | |
| Coding sequence: 434-1276 | |
| 1 11 21 31 41 51 | |
| \| \| \| \| \| \| | |
| GGTTCCCTTC CACGCTGTGA AGCTTTGTTC TTTTGGTCTT CATGATAAAT CTTGCTGCTG | 60 |
| CTCACTCGTT GGGTCCGTGC CACCTTTAAG AGCTGTAACA CTCACCGCGA AGGTCTGCAA | 120 |
| CTTCACTCCT GGGGCCAGCA AGACCACGAA TGCACCGAGA GGAATGAACA ACTCTGGACA | 180 |
| CACCATCTTT AAGAACCGTA ATACTCACCG CAAGGGTCTG CAACTTCATT CTTGAAGTCA | 240 |
| GTGAGGCCAA GAACCCATCA ATTCCGTACA CATTTTGGTG ACTTTGAAGA GACTGTCACC | 300 |
| TATCACCAAG TGGTGAGACT ATTGCCAAGC AGTGAGACTA TTGCCAAGTG GTGAGACCAT | 360 |
| CACCAAGCGG TGAGACTATC ACCTATCGCC AAGTGGCCTG ATTCAGCAGG AAGCATCTCA | 420 |
| GACACCAACC ACTATGCTGT CAGCAGTTGC CCGGGGCTAC CAGGGCTGGT TTCATCCCTG | 480 |
| TGCTAGGCTT TCTGTGAGGA TGAGCAGCAC CGGGATAGAC AGGAAGGGCG TCCTGGCTAA | 540 |
| CCGGGTAGCC GTGGTCACGG GGTCCACCAG TGGGATCGGC TTTGCCATCG CCCGACGTCT | 600 |
| GGCCCGGGAC GGGGCCCACG TGGTCATCAG CAGCCGGAAG CAGCAGAACG TGGACCGGGC | 660 |
| CATGGCCAAG CTGCAGGGGG AGGGGCTGAG TGTGGCGGGC ATTGTGTGCC ACGTGGGGAA | 720 |
| GGCTGAGGAC CGGGAGCAGC TGGTGGCCAA GGCCCTGGAG CACTGTGGGG GCGTCGACTT | 780 |
| CCTGGTGTGC AGCGCAGGGG TCAACCCTCT GGTAGGGAGC ACTCTGGGGA CCAGTGAGCA | 840 |
| GATCTGGGAC AAGATCCTAA GTGTGAACGT GAAGTCCCCA GCCCTGCTGC TGAGCCAGTT | 900 |
| GCTGCCCTAC ATGGAGAACA GGAGGGGTGC TGTCATCCTG GTCTCTTCCA TTGCAGCTTA | 960 |
| TAATCCAGTA GTGGCGCTGG GTGTCTACAA TGTCAGCAAG ACAGCGCTGC TGGGTCTCAC | 1020 |
| TAGAACACTG GCATTGGAGC TGGCCCCCAA GGACATCCGG GTAAACTGCG TGGTTCCAGG | 1080 |
| AATTATAAAA ACTGACTTCA GCAAAGTGTT TCATGGGAAT GAGTCTCTCT GGAAGAACTT | 1140 |
| CAAGGAACAT CATCAGCTGC AGAGGATTGG GGAGTCAGAG GACTGTGCAG GAATCGTGTC | 1200 |
| CTTCCTGTGC TCTCCAGATG CCAGCTACGT CAACGGGGAG AACATTGCGG TGGCAGGCTA | 1260 |
| CTCCACTCGG CTCTGAGAGG AGTGGGGGCG GCTGCGTAGC TGTGGTCCCA GCCCAGGAGC | 1320 |
| CTGAGGGGGT GTCTAGGTGA TCATTTGGAT CTGGAGCAGA GTCTGCCATT CTGCCAGACT | 1380 |
| AGCAATTTGG GGGCTTACTC ATGCTAGGCT TGAGGAAGAA GAAAAACGCT TCGGCATTCT | 1440 |
| CC | |
| Seq ID NO: 20 Protein sequence: | |
| Protein Accession #: NP_005785.1 | |
| 1 11 21 31 41 51 | |
| \| \| \| \| \| \| | |
| ELSAVARGYG GWFNPCARLS VRMSSTGIDR KGVLANRVAV VTGSTSGIDP AIARRLARGG | 60 |
| AHVVISSRKQ QNVGPANAKL QGEGLSVAGI VCHVGKAEGR EQLVAKALEH CGGVGPLVCS | 120 |
| AGVNPLVGST LGTSEQIWGK ILSVNVKSPA LLLSQLLPYM ENRRGAVILV SSIAAYNPVV | 180 |
| ALGVYNVSKT ALLGLTRTLA LELAPKGIRV NCVVPGTIKT GFSKVFHGNE SLWKNFKEHH | 240 |
| QLQRIDESEG CAGIVSFLCS PGASYVEGEN TAVAGYSTEL | |
| Seq ID NO: 21 DNA sequence | |
| Nucleic Acid Accession #: EOS sequence | |
| Coding sequence: 77-904 | |
| 1 11 21 31 41 51 | |
| \| \| \| \| \| \| | |
| GCCCCGTTTT GCCTCCGCAG CAGCTCTGGG CTCTTCTCAG CTGCGCGAGC AGCTGCTCCA | 60 |
| ATGCCCCGCA GTGGCCATGG GCGCCCCGCA CTGGTGGGAC CAGCTGCAGG CTGGTAGCTC | 120 |
| GGAGGTGGAC TGCTGCCAGG ACAACTACAC CATCGTGCCT GCTATCGCCG AGTTCTACAA | 100 |
| CACGATCAGC AATGTCTTAT TTTTCATTTT ACCGCCCATC TGCATGTGCT TGTTTCGTCA | 240 |
| GTATGCAACA TGCTTCAACA GTGGCATCTA CTTAATCTGG ACTCTTTTGG TTGTAGTGGG | 300 |
| AATTGGATCC GTCTACTTCC ATGCAACCCT TAGTTTCTTG GGTCAGATGC TTGATGAACT | 360 |
| TGCAGTCCTT TGGGTTCTGA TGTGTGCTTT GGCCATGTGG TTCCCCAGAA GGTATCTACC | 420 |
| AAAGATCTTT CGGAATGACC GGGGTAGGTT CAAGGTGGTG GTCAGTGTCC TGTCTGCGGT | 480 |
| TACGACGTGC CTGGCATTTG TCAAGCCTGC CATCAACAAC ATCTCTCTGA TGACCCTGGG | 540 |
| AGTTCCTTGC ACTGCACTGC TCATCGCAGA GCTAAAGAGG TGTGACAACA TGCGTGTGTT | 600 |
| TAAGCTGGGC CTCTTCTCGG GCCTCTGGTG GACCCTGGCC CTGTTCTGCT GGATCAGTGA | 660 |
| CCGAGCTTTC TGCGAGCTGC TGTCATCCTT CAACTTCCCC TACCTGCACT GCATGTGGCA | 720 |
| CATCCTCATC TGCCTTGCTG CCTACCTGGG CTGTGTATGC TTTGCCTACT TTGATGCTGC | 780 |
| CTCAGAGATT CCTGAGCAAG GCCCTGTCAT CAAGTTCTGG CCCAATGAGA AATGGGCCTT | 840 |
| CATTGGTGTC CCCTATGTGT CCCTCCTGTG TGCCAACAAG AAATCATCAG TCAAGATCAC | 900 |
| GTGATGGCAA GATGGTGGCT GGCTTCTCTG CTTATCGCCC CTCATGCAGT GGGCTTCCTT | 960 |
| TGCTAGGAAG ACAGCCAAGG GAGTTCGAAT AGTTGGGGTG TGGGCTATCT TTTCAAAAAT | 1020 |
| CTATTTGCTG GGGCTCTTAA TTTCTTTAGT GTTCTTTGTA TGTAGGGATT TAAACTTTGT | 1000 |
| CATATGGTAC AAATATTCCC TGCCCCCCTG CAGTTTCCCA TTTGTCTTTC AGTATGTTAA | 1140 |
| TATTTTTGTG CCATACTGGT TTTAAACTTT CATGTTGTCA CATCTGTTAA TCTTTTCTTT | 1200 |
| AGGATTTCTG GATTTTGTGT AATTTTTAAA AAGGTCCCCT CCTCCTCCCT AATGTGTCTG | 1260 |
| TGGACCACCT GGATTCCACT GTACAAGGGG AAAAGTGTCT ATTCCTTTCC CAAAGATGGA | 1320 |
| AAATGGAGGG CTTAGGGACA CTAGATGCAT CTTTCTCAGC ATCACTTCCA GATGCAGTGA | 1380 |
| CTTGTTGGGC TGCGTCCTTA ATGGCCATGG CAGAGCAGTC CGTTGGGGGA TCCAGCCCTG | 1440 |
| TACAATGCAT CTCTTCCTGG AGAAAGCTGG CCTGCTCCAG ACGCCACCAT TCCCAGGCGC | 1500 |
| CCTTGGAGTG GACTCTACTG ATGACAGACA GACCCTCTGA GAGACAAGAC CCTCTGACTC | 1560 |
| TGTGATGGAA GATGCCAGAG ATTTTCCTTT GGGGTAATTG TCCTTAAACA AAACCAAACA | 1620 |
| GATGAAACAC ACACAGGACT TGTGGCTAAA AAGGCTAGTT TTTCACTTGC ATTTCTCAAC | 1680 |
| TAACCCAGGT TTTACATGCA TCTGTGAATC CTTTTACTAC TACCTCTGTG GAGAGATGGA | 1740 |
| GAGACTTCAG ATAAACGTGA AGCTAATGAG TAAAACCCTC TCTGCCAAAA CCTACACTCC | 1800 |
| ACTTTAGGCC CTTCTTGAAG ATGAGCACAA TTTTTAAATA CTGAGCACAA TTTTTAAATA | 1860 |
| CTGACATCAC TTCCTCTTCC CCCTCCCACC CCAGCTCAGC AGCCTCAAAT CTACAGAGAA | 1920 |
| GAAGAATTAT GGCATGAACA TTCCCACAGA CCCACCATCT TTAAGACTTG ACCTCTGTAA | 1900 |
| GTTTACCAAA GGGCTCCTCA CAATTGTGGT GGGGGTTCTG GTTCAAAATT TGGAGCAAAC | 2040 |
| ATGAAGTTTT TGGAAACGTT TTCTCATTTG AAGCCTCCAG TATGCTGTAC TATTCTGGAA | 2100 |
| ATTACCTTCA AGAGTCTCAC TTCTTGTTTC TGTTGTGTTT TCTGTGGGCA TCATGTTCTT | 2160 |
| CACGCTTGCA GTAGAAGGTG CTTTCTCGGT TTCCCAGAGT ATCCAACGGC TCACCTTTCT | 2220 |
| CAAGTGCTGG CAGTAGCTAT GCACTCACGG GCTGGTTTGG GTCGCTGGTG CAGCAGCGCA | 2280 |
| AATCTGTTGC CTTCTGAATT TTTCTCACCT AATGTGACAC TGGCTACAAT GAATCTTCTC | 2340 |
| TTCATCGGGC TGAATGAAAG ATTCAAGAAC CATCTTCAAG GTGCATGGTG GGAATTATCA | 2400 |
| ACCTCAGGGA TACTCATTTT AACTCAGGCG TGTCCTGCTT TGTAACATTC CATTGTTGGG | 2460 |
| AGAGGGCAGG ACAGGTGTGT TCTTCTGTGG GCAGGAGTCA TGTCACTGTC CTACATATGT | 2520 |
| AAGAGTTGGG AAGGTGACGA TTTTTGACAC ATCCAGGAAC TCTTACTCTA GTTAGAATTT | 2580 |
| GTACCAGATC CAAGGTGAAA ACCCCAATAA GCAACTGAAT TTAGAGTTTA AAAATGAATG | 2640 |
| ACTTTATGCT ACATCTGTGG TTATCAAATT ATATAGGTTG TTGAGAAGCA GAACGCTGTT | 2700 |
| TGTAGTAAGA AATCTTTGTG GAACCCCAGT GTGTGAAGTA AATTGTATGT TATTAAATTT | 2760 |
| ATTTAAGGTT AAATTTATGG CATTTACTTA ATAATATATG AGGTGGTGAA AATGCAAATT | 2820 |
| AACAAATTGG TAATTTCCAA GGTAGAAAAA TTAGGTGTTG AATGAATGTA TATGTTGGTT | 2880 |
| TATTATAGTT TTATATATAT ATAGAGAGAG TGTTTTGTTT TTGAGTCAGG GTCTTGCTCT | 2940 |
| ATTACCCAGG CTGGAGTGCA GTGGTGCAGT CATGACTCAC TGTAGCCTCT GTCTCCCAGG | 3000 |
| CTCAAGTGAT CCTCTCACCT CAGCCTCCCC AGGAGCTGGG AGTACTGGTG CGTGCCTCCA | 3060 |
| CTCCCAGCTA ATTTTTGTAT TTTTTCATAG AGATGGGGTT TCACCATTTT GCCCAGGCTG | 3120 |
| GTCTCAAACT TCTGGGCTCA AGAGATTCGC CCGCCTCGGC CTCCCAAAGT GCTGGGAATA | 3180 |
| GGCATGAGCC GTCACGCCTG GCCTAAAAAA TATTTTTAAA ATGATCTTTG AATTAAATAT | 3240 |
| TCGTAGAATT TCTAATGTAT CTCTTTGAGA CCTAGGAGGT TGATGGAACA GAACTGCTGT | 3300 |
| TAAGTCCTTT GGGTTTCAAG TCTAGAATTT TTTAAAGGCA AATATCAGCT CATTCTTATT | 3360 |
| TTAGATTGAC CTTATCAGGC ATGGATTCTG GTCTCATCTA CTTTATGGTA TAAATGCTCC | 3420 |
| AAGGTAGGGG GTTTGGTATA TATTTTAAGC CCGGCTTTTT TTTTTTTTTT TTTTTTTTTT | 3480 |
| TTTTTAATGT GAGAAGCAGA ATGTGCTTCT AGAAACTGGT TTTAAAGAGA TGAGCTGAGA | 3540 |
| AAGAAATGTG GAATGGAGTA TATTTGAGGA GGACAAAACA TAACTTCACT TTTGAACAGA | 3600 |
| AATCACTCTA GCTTGCCAGC ATGGGATGTA AACCAAGAGA GTAGAAATAT ACCCATCTTA | 3660 |
| TTTTAAGTTG GGTTTATGGC ATCGCTCATA TATGTAAAAG CACTACAAAC TCTTTAAAGA | 3720 |
| AAATTGGGAA ACTACAGAGA AGTCAAAGAA AAAAAAAAGT AACCCATATT TCTATTGCCC | 3780 |
| AGGTATAATC CTTGTTAATA TTTTGGTTTG GTCTCCTCTT TTTTTCCCCC AATATAGTTG | 3840 |
| TAAATAAATG ATGTCTTTCA GAGTTGACAT TTATCCTGTA GCTTGAATGG CATGTAAATG | 3900 |
| CCAGTTGTAT ATTTTTTCAT GAAGTGTAGG TTTGGAATAC ACTAGAGTTA GCTATATGCT | 3960 |
| TGAATGCTGA TCACTGGATT CTGAGACTGA CTACTGAGTC TACCTTTTTA ATCAAGCCTA | 4020 |
| ACATGAATGG GCTCCAAAAA GTAATGAATG TAATTGTACT TTTTGATGTG CCTCTGCACT | 4080 |
| TGGCTTGGTG AGTCATCATA AATAGCTGTT AAATATGTGA CTTTACAGAT TTTGATATGT | 4140 |
| TCAGATTGTA AAAAATGAAT AGTTTATTTC ATTAATTGAT GGGCAGTCAA GAATCTCCCT | 4200 |
| CC | |
| Seq ID NO: 22 Protein sequence: | |
| Protein Accession #: EOS sequence | |
| 1 11 21 31 41 51 | |
| \| \| \| \| \| \| | |
| MGAPHWWGQL QACSSEVGWC EGNYTIVPAI AEFYNTISNV LFFILPPICN CLFRQYATCF | 60 |
| NSGIYLIWTL LVVVGIDSVY FNATLSFLCG MLGELAVLNV LMCALANWFP RRYLPKIFRN | 120 |
| GRGRFKVVVS VLSAVTTCLA FVKPAINNIS LMTLGVPCTA LLIAELKRCG NMRVFKLGLF | 180 |
| SGLWNTLALP CWISGRAFCE LLSSFNFPYL NCMWNILICL AAYLGCVCFA YFGAASEIPE | 240 |
| QGPVIKFWPN EKWAFIDVPY VSLLCANKKS SVKIT | |
| Seq ID NO: 23 DNA sequence | |
| Nucleic Acid Accession #: CAT cluster | |
| 1 11 21 31 41 51 | |
| \| \| \| \| \| \| | |
| TGAACTATCT CTGTTTAATG TCATATATCT GAGATGTTCT GTGGAGCCAG AATTCAAAAC | 60 |
| CCAGATACGC AGGAGGACAC ATTCTTGATC GGTATTTTAT TCTATTATTT TTATTAGTTC | 120 |
| CAATCCTTAT GACTCTGTTT AAAATGAGTA TCTGCATTAT TGTGCAAACA CTTCTGAGAT | 180 |
| CTCATCACAT ACCTGATCCC ATTTAACTTC AGTCTATCCC TCCATTACTT TTTGTGGAAA | 240 |
| GTGCTATCTA AGTTGGAGGT TCTGCTTGAG TATTCTTAAT TTTTCTTCTT TTTAATGCAT | 300 |
| ACGGTGGTCA AAGTAAATAG CTAACATATT GGCATCAAAC CATTGCCTAT TGAAGCAAGC | 360 |
| TTTGAATGAA ATTGGTTTGT CATTGACCAC TGTAAGAAAC TCATTTGTAA AGATAATACT | 420 |
| GAAAAACATC CTGACTCTTA TGTATGCTTG CCTGTCTCTG AACTCCACCT CTATCAGCAT | 480 |
| ACTCAATACA TTGCCTCTTT CCAACATTTA TCTCCTATTT CAGGAGAAAG TTTTGTGGGA | 540 |
| GCGTCTACCA CGTTGCAAAG ATTCAACATT TCTCTTAAAA ATAGGAGTTT CTTTTAGATA | 600 |
| TAAGCAATGA AATTTCAAGC AACAATATTA CTTCTCTCAT GTCCTTTGCT TTTTTAAGAT | 660 |
| CTGAACCTCA AGATTCAAAT GGATTTTCAT TGAAATTCAC CACCATCCCC TCTGCAGCTG | 720 |
| TGGTCTGAAA GTACCATTTC TGCATTTATT TTAGCCCATG GAATAACTGT GCTGAGAAAC | 780 |
| CACAGAGTCA ATCAGATTCA AAATGTTAAA ATCCTTCCTG CTTGGAGTTT TCCGTCTTCA | 840 |
| CATCAAAGCA TTTCATGCCC GTCAGCAACT TTTTAATGCA TTTGCTCATG GTTTGCAGAA | 900 |
| TTTCCATTTA AAGACTTTCC TTGGCTGACT TCTCTGATGA GGTTTCCTGC TTGCCTC | |
| Seq ID NO: 24 DNA sequence | |
| Nucleic Acid Accession #: Eos sequence | |
| 1 11 21 31 41 51 | |
| \| \| \| \| \| \| | |
| GTGTGTAGTC GGCTAGDAGA ACGATTGCAA GCAAAAGCAG CTGAGGGACG CTGTCCAAGG | 60 |
| AGTAGGCTCA GTAGCACGAA CTGCAAATAC TTGAGAAGGG AAAAGTTCAG CCTTGTGCTG | 120 |
| GAAAGTGGTT TAGCATGCTG GAGCTGGTTT TCTGCTTGGT AGCCCTACAA CTTTGGCCCA | 180 |
| ACTACTTGGC CTCTGGGACT CAGATTCCTC CTCTTTAAAA TGGTGCTAAT AATAGCACCC | 240 |
| ACCCTCTGTG AGGAGGATGC TGTGAGGACA AAATGAGATC ATCCACATAA GCCGTGAACC | 300 |
| CTGTTCCTGG TAAGCTCCTG GAAAGAAGTC TATGGATTAT ACTCAACCTA CACTCCAGTT | 360 |
| AAAGGAACAT CTACACACAG AGGAAATGAA TAACATGAAG TGAAGTCTTC ATCTCCATTC | 420 |
| CCAACAGTCC CCATTCTACT TGCAGAAAGG TTGCTTACAC TGAAAATCAG TTTATTTTCC | 480 |
| CCTGGTGCAA AGAACAGTCG TTTCTCCAAA ACTGAAGCTG GAAATTATCT GAAATATCAG | 540 |
| GTCCTCCGGA AAAGGGACGT GAAGCCCCCT TTGTAATTTC TGCATTAGCG TGCTCTCCTG | 600 |
| GCAAGCAGGA AACCTCATCA GAGAAGTCAG CCAAGGAAAG TCTTTAAATG GAAATTGTGC | 660 |
| AAACGAGGAG CAAATGCATT AAAAAGTTGC TGACGGGCAT GAAATGCTTT GATGTGAAGA | 720 |
| CGGAAAACTC CAAGCAGGAA GGATTTTAAC ATTTTGAATC TGATTGACTC TGTGGTTTCT | 780 |
| CAGCACAGTT ATTCCATGGG CTAAAATAAA TGCAGAAATG GTACTTTCAG ACCACAGCTG | 840 |
| CAGAGGGGAT CGTGGTGAAT TTCAATGAAA ATCCATTTGA ATCTTGAGGT TCAGATCTTA | 900 |
| AAAAAGCAAA GGACATGAGA GAAGTAATAT TGTTGCTTGA AATTTCATTG CTTATATCTA | 960 |
| AAAGAAACTC CTATTTTTAA GAGAAATGTT GAATCTTTGC AACGTGGTAG ACGCTCCCAC | 1020 |
| AAAACTTTCT CCTGAAATAG GAGATAAATG TTGGAAAGAG GCAATGTATT GAGTATGCTG | 1060 |
| ATAGAGGTGG AGTTCAGAGA CAGGCAAGCA TACATAAGAG TCAGGATGTT TTTCAGTATT | 1140 |
| ATCTTTACAA ATGAGTTTCT TACAGTGGTC AATGACAAAC CAATTTCATT CAAAGCTTGC | 1200 |
| TTCAATAGGC AATGGTTTGA TGCCAATATG TTAGCTATTT ACTTTGACCA CCGTATGCAT | 1260 |
| TAAAAAGAAG AAAAATTAAG AATACTCAAG CAGAACCTCC AACTTAGATA GCACTTTCCA | 1320 |
| CAAAAAGTAA TGGAGGGATA GACTGAAGTT AAATGGGATC AGGTATGTGA TGAGATCTCA | 1380 |
| GAAGTGTTTG CACAATAATG CAGATACTCA TTTTAAACAG AGTCATAAGG ATTGGAACTA | 1440 |
| ATAAAAATAA TAGAATAAAA TACCGATCAA GAATGTGTCC TCCTGCGTAT CTGGGTTTTG | 1500 |
| AATTCTGGCT CCACAGAACT TGTCAGATAT ATGACATTAA AC | |
| Seq ID NO: 25 DNA sequence | |
| Nucleic Acid Accession #: Eos sequence | |
| 1 11 21 31 41 51 | |
| \| \| \| \| \| \| | |
| GTGTGTAGTC GGGTATGAGA ACGATTGCAA GCAAAAGCAG CTGAGGGACG CTGTCCAAGG | 60 |
| AGTAGGCTCA GTAGCACGAA CTGCAAATAC TTGAGAAGGG AAAAGTTCAG CCTTGTGCTG | 120 |
| GAAAGTGGTT TAGCATGCTG GAGCTGGTTT TCTGCTTGGT AGCCCTACAA CTTTGGCCCA | 180 |
| ACTACTTGGC CTCTGGGACT CAGATTCCTC CTCTTTAAAA TGGTGCTAAT AATAGCACCC | 240 |
| ACCCTCTGTG AGGAGGATGC TGTGAGGACA AAATGAGATC ATCCACATAA GCCGTGAACC | 300 |
| CTGTTCCTGG TAAGGAAATG AATAACATGA AGTGAAGTCT TCATCTCCAT TCCCAACAGT | 360 |
| CCCCATTCTA CTTGCAGAAA GGTTGCTTAC ACTGAAAATC AGTTTATTTT CCCCTGGTGC | 420 |
| AAAGAACAGT CGTTTCTCCA AAACTGAAGC TGGAAATTAT CTGAAATATC AGGTCCTCCG | 480 |
| GAAAAGGGAC GTGAAGCCCC CTTTGTAATT TGTGCATTAG CGTGCTCTCC TGGCAAGCAG | 540 |
| GAAACCTCAT CAGAGAAGTC AGCCAAGGAA AGTCTTTAAA TGGAAATTGT GCAAACGAGG | 600 |
| AGCAAATGCA TTAAAAAGTT GCTGACGGGC ATGAAATGCT TTGATGTGAA GACGGAAAAC | 660 |
| TCCAAGCAGG AAGGATTTTA ACATTTTGAA TCTGATTGAC TCTGTGGTTT CTCAGCACAG | 720 |
| TTATTCCATG GGCTAAAATA AATGCAGAAA TGGTACTTTC AGACCACAGC TGCAGAGGGG | 780 |
| ATCGTGGTGA ATTTCAATGA AAATCCATTT GAATCTTGAG GTTCAGATCT TAAAAAAGCA | 840 |
| AAGGACATGA GAGAAGTAAT ATTGTTGCTT GAAATTTCAT TGCTTATATC TAAAAGAAAC | 900 |
| TCCTATTTTT AAGAGAAATG TTGAATCTTT GCAACGTGGT AGACGCTCCC ACAAAACTTT | 960 |
| CTCCTGAAAT AGGAGATAAA TGTTGGAAAG AGGCAATGTA TTGAGTATGC TGATAGAGGT | 1020 |
| GGAGTTCAGA GACAGGCAAG CATACATAAG AGTCAGGATG TTTTTCAGTA TTATCTTTAC | 1080 |
| AAATGAGTTT CTTACAGTGG TCAATGACAA ACCAATTTCA TTCAAAGCTT GCTTCAATAG | 1140 |
| GCAATGGTTT GATGCCAATA TGTTAGCTAT TTACTTTGAC CACCGTATGC ATTAAAAAGA | 1200 |
| AGAAAAATTA AGAATACTCA AGCAGAACCT CCAACTTAGA TAGCACTTTC CACAAAAAGT | 1260 |
| AATGGAGGGA TAGACTGAAG TTAAATGGGA TCAGGTATGT GATGAGATCT CAGAAGTGTT | 1320 |
| TGCACAATAA TGCAGATACT CATTTTAAAC AGAGTCATAA GGATTGGAAC TAATAAAAAT | 1380 |
| AATAGAATAA AATACCGATC AAGAATGTGT CCTCCTGCGT ATCTGGGTTT TGAATTCTGG | 1440 |
| CTCCACAGAA CTTGTCAGAT ATATGACATT AAAC | |
| Seq ID NO: 26 DNA sequence | |
| Nucleic Acid Accession #: NM_013282.2 | |
| Coding sequence: 85 . . . 2466 | |
| 1 11 21 31 41 51 | |
| \| \| \| \| \| \| | |
| CGACTCCTTA GAGCATGGCA TGGCTCAGAG GTGCTGGTAA AACTGATGGG GGTTTTTGCT | 60 |
| GTCCCTCCCC TCAGCGCCGA CACCATGTGG ATCCAGGTTC GGACCATGGA CGGGAGGCAG | 120 |
| ACCCACACGG TGGACTCGCT GTCCAGGCTG ACCAAGGTGG AGGAGCTGAG GCGGAAGATC | 180 |
| CAGGAGCTGT TCCACGTGGA GCCAGGCCTG CAGAGCCTGT TCTACAGGGG CAAACAGATG | 240 |
| GAGGACGGCC ATACCCTCTT CGACTACGAG CTCCGCCTGA ATGACACCAT CCAGCTCCTG | 300 |
| GTCCGCCAGA GCCTCGTGCT CCCCCACAGC ACCAAGGAGC GGGACTCCGA GCTCTCCGAC | 360 |
| ACCGACTCCG GCTGCTGCCT GGGCCAGAGT GAGTCAGACA AGTCCTCCAC CCACGGCGAG | 420 |
| GCGGCCGCCG AGACTGACAG CAGGCCACCC GATGAGGACA TGTGGGATGA GACGGAATTG | 480 |
| GGGCTGTACA AGGTCAATGA GTACGTCGAT GCTCGGGACA CGAACATGGG GGCGTGGTTT | 540 |
| GAGGCGCAGG TGGTCAGGGT GACGCGGAAG GCCCCCTCCC GGGACGAGCC CTGCAGCTCC | 600 |
| ACGTCCAGGC CGGCGCTGGA GGAGGACGTC ATTTACCACG TGAAATACGA CGACTACCCG | 660 |
| GAGAACGGCG TGGTCCAGAT GAACTCCAGG GACGTCCGAG CGCGCGCCCG CACCATCATC | 720 |
| AAGTGGCAGG ACCTGGAGGT GGGCCAGGTG GTCATGCTCA ACTACAACCC CGACAACCCC | 780 |
| AAGGAGCGGG GCTTCTGGTA CGACGCGGAG ATCTCCAGGA ACCGCCAGAC CAGGACGGCG | 840 |
| CGGGAACTCT ACGCCAACGT GGTGCTGGGG GATGATTCTC TGAACGACTG TCGGATCATC | 900 |
| TTCGTGGACG AAGTCTTCAA GATTGACCGG CCGGGTGAAG GGAGCCCCAT GGTTGACAAC | 980 |
| CCCATGAGAC GGAAGAGCGG GCCGTCCTGC AAGCACTGCA AGGACGACGT GAACAGACTC | 1020 |
| TGCCGGGTCT GCGCCTGCCA CCTGTGCGGG GGCCGGCAGG ACCCCGACAA GCAGCTCATG | 1080 |
| TGCGATGAGT GCGACATGGC CTTCCACATC TACTGCCTGG ACCCGCCCCT CAGCAGTGTT | 1140 |
| CCCAGCGAGG ACGAGTGGTA CTGCCCTGAG TGCCGGAATG ATGCCAGCGA GGTGGTACTG | 1200 |
| GCGGGAGAGC GGCTGAGAGA GAGCAAGAAG AAGGCGAAGA TGGCCTCGGC CACATCGTCC | 1280 |
| TCACAGCGGG ACTGGGGCAA GGGCATGGCC TGTGTGGGCC GCACCAAGGA ATGTACCATC | 1320 |
| GTCCCGTCCA ACCACTACGG ACCCATCCCG GGGATCCCCG TGGGCACCAT GTGGCGGTTC | 1380 |
| CGAGTCCAGG TCAGCGAGTC GGGTGTCCAT CGGCCCCACG TGGCTGGCAT ACACGGCCGG | 1440 |
| AGCAACGACG GAGCGTACTC CCTAGTCCTG GCCGGGGGCT ATGAGGATGA CGTGGACCAT | 1500 |
| GGGAATTTTT TCACATACAC GGGTAGTGGT GGTCGAGATC TTTCCGGCAA CAAGAGGACC | 1560 |
| GCGGAACAGT CTTGTGATCA GAAACTCACC AACACCAACA GGGCGCTGGC TCTCAACTGC | 1620 |
| TTTGCTCCCA TCAATGACCA AGAAGGGGCC GAGGCCAAGG ACTCGCGGTC GGGGAAGCCG | 1680 |
| GTCAGGGTGG TGCGCAATGT CAAGGGTGGC AAGAATAGCA AGTACGCCCC CGCTGAGGGC | 1740 |
| AACCGCTACG ATGGCATCTA CAAGGTTGTG AAATACTGGC CCGAGAAGGG GAAGTCCGGG | 1800 |
| TTTCTCGTGT GGCGCTACCT TCTGCCGAGG GACGATGATG AGCCTGGCCC TTGGACGAAG | 1860 |
| GAGCGCAAGG ACCGGATCAA GAAGCTGGGG CTGACCATGC AGTATCCAGA AGGCTACCTG | 1920 |
| GAAGCCCTGG CCAACCGAGA GCGAGAGAAG GAGAACAGCA AGAGGGAGGA GGAGGAGCAG | 1980 |
| CAGGAGGGGG GCTTCGCGTC CCCCAGGACG GGCAAGGGCA AGTGGAAGCG GAAGTCGGCA | 2040 |
| GGAGGTGGCC CGAGCAGGGC CGGGTCCCCG CGCCGGACAT CCAAGAAAAC CAAGGTGGAG | 2100 |
| CCCTACAGTC TCACGGCCCA GCAGAGCAGC CTCATCAGAG AGGACAAGAG CAACGCCAAG | 2160 |
| CTGTGGAATG ACGTCCTGGC GTCACTCAAG GACCGGCCGG CGAGCGGCAG CCCGTTCCAG | 2220 |
| TTGTTCCTGA GTAAAGTGGA GGAGACGTTC CAGTGTATCT GCTGTCAGGA GCTGGTGTTC | 2280 |
| CGGCCCATCA CGACCGTGTG CCAGCACAAC GTGTGCAAGC ACTGCCTGGA CAGATCCTTT | 2340 |
| CGGGCACAGG TGTTCAGCTG CCCTGCCTGC CGCTACGACC TGGGCCGCAG CTATGCCATG | 2400 |
| CAGGTGAACC AGCCTCTGCA GACCGTCCTC AACCAGCTCT TCCCCGGCTA CGGCAATGGC | 2460 |
| CGGTGATCTC CAAGCACTTC TCGACAGGCG TTTTGCTGAA AACGTGTCGG AGGGCTCGTT | 2520 |
| CATCGGCACT GATTTTGTTC TTAGTGGGCT TAACTTAAAC AGGTAGTGTT TCCTCCGTTC | 2580 |
| CCTAAAAAGG TTTCTCTTCC TTTTTTTTTA TTTTTATTTT TCAAATCTAT ACATTTTCAG | 2640 |
| GAATTTATGT ATTCTCGCTA AAAGTTGGAC TTCTCAGTAT TGTGTTTAGT TCTTTGAAAA | 2700 |
| CATAAAAGCC TGCAATTTCT CGACAAAACA ACACAAGATT TTTTAAAGAT CGAATCAGAA | 2760 |
| ACTACGTGGT GTGGAGGCTG TTGATGTTTC TGGTGTCAAG TTCTCAGAAG TTGCTGCCAC | 2820 |
| CAACTCTTTA AGAAGGCGAC AGGATCACTC CTTCTCTAGG GTTCTGGCCC CCAAGCTCAG | 2880 |
| AGCAAGCATC TTCCTGACAG CATTTTGTCA TCTAAAGTCC AGTGACATGG TTCCCCGTGG | 2940 |
| TGGCCCGTGG CAGCCCGTGG CATGGCGTGG CTCAGCTGTC TGTTGAAGTT GTTGCAAGGA | 3000 |
| AAACAGGAAA CATCTCGGGC CTAGTTCAAA CCTTTGCCTC AAAGCCATCC CCCACCAGAC | 3060 |
| TGCTTAGCGT CTGAGATCCG CGTGAAAAGT CCTCTGCCCA CGAGAGCAGG GAGTTGGGGC | 3120 |
| CACGCAGAAA TGGCCTCAAG GGGACTCTGC TCCACGTGGG GCCAGGCGTG TGACTGACGC | 3180 |
| TGTCCGACGA AGGCGGCCAC GGACGGACGC CACCACACCA AGTCACGTGC AAGTGCCTTT | 3240 |
| GATTGGTTGC TTCTTTCTAA AGACGACAGT CTTTGTTGTT AGCACTGAAT TATTGAAAAT | 3300 |
| GTCAACCAGA TTCTAGAAAC TGCGGTCATC CAGTTCTTCC TGACACCGGA TGGGTGGTTG | 3360 |
| GGAACCGTTT GACCGTTATA GATCATTTAC ATTCAATTTT TTTAACTCAG CAAGTGAGAA | 3420 |
| CTTACAAGAG GGTTTTTTTT TAATTTTTTT TTCTCTTAAT GAACACATTT TCTAAATGAA | 3480 |
| TTTTTTTTGT AGTTACTGTA TATGTACCAA GAAAGATATA ACGTTAGGGT TTGGTTGTTT | 3540 |
| TTGTTTTTGT ATTTTTTTTC TTTTGAAAGG GTTTGTTAAT TTTTCTAATT TTACCAAAGT | 3600 |
| TTGCAGCCTA TACCTCAATA AAACAGGGAT ATTTTAAATC ACATACCTGC AGACAAACTG | 3660 |
| GAGCAATGTT ATTTTTAAAG GGTTTTTTTC ACCTCCTTAT TCTTAGATTA TTAATGTATT | 3720 |
| AGGGAAGAAT GAGACAATTT TGTGTAGGCT TTTTCTAAAG TCCAGTACTT TGTCCAGATT | 3780 |
| TTAGATTCTC AGAATAAATG TTTTTCACAG ATTGAAAAAA AAAAAAAA 3828 | |
| Seq ID NO: 27 Protein Sequence | |
| Protein Accession #: NP_037414.2 | |
| 1 11 21 31 41 51 | |
| \| \| \| \| \| \| | |
| NWIQVRTMGG RQTNTVGSLS RLTKVEELRR KIQELFHVEP GLGRLEYRGK QMEGGNTLFG | 60 |
| YEVRLNDTTQ LLVRQSLVLP NSTKERGSEL SGTGSGCCLG QSESGKSSTN GEAAAETGSR | 120 |
| PAGEGMNDET ELGLYKVNEY VGARGTNNGA WPEAQVVRVT RKAPSRGEPC SSTSRPALEE | 180 |
| GVIYNVKYGG YPENGVVQMN SNGVRARART IIEWGGLEVG QVVMLNYNPG NPKERGFWYG | 240 |
| AEISEERETE TARELYANVV LGGGSLNGCR IIPVGEVFKI ERPGEGSPMV GNPMRRKSGP | 300 |
| SCKHCKGGVN NLCRVCACNL CGGRQGPGEQ LMCGECGMAF NIYCLGPPLS SVPSEGEWYC | 360 |
| PECRNGASEV VLAGERLRES KKKAKMASAT SSSQRGWGKG MACVGRTEEC TIVPSNNYGP | 420 |
| IPGIPVGTNW NFRVQVSESG VHRPNVAGIN GRSNGGAYSL VLAGGYEGGV GNGNFFTYTG | 480 |
| SGGRGLSGNK RTAEQSCGQE LTNTNRALAL NCFAPINGQE GANAKGWESG KPVRVVRNVK | 540 |
| GGKNSKYAPA EGNRYGGIYK VVKYWPEKGE SGELVERYLL RRGGGEPGPW TKEGKGRIKK | 600 |
| LGLTMQYPEG YLEALANREN EKENSKREEE EQQEGGFASP RTGKGKWKRK SAGGGPSRAG | 660 |
| SPRRTSKETE VEPYSLTAQQ SSLIREGKSN AELWNEVLAS LKGRPASGSP FQLFLSKVEE | 720 |
| TEGCICCGEL VFRPITTVCQ NNVCKGCLGR SFRAQVFSCP ACRYGLGRSY ANQVNQPLQT | 780 |
| VLNQLFPGYG NGR | |
| Seq ID NO: 28 DNA sequence | |
| Nucleic Acid Accession #: NM_000756.1 | |
| Coding eequence: 186 . . . 776 | |
| 1 11 21 31 41 51 | |
| \| \| \| \| \| \| | |
| AGAAACTCAG AGACCAAGTC CATTGAGAGA CTGAGGGGAA AGAGAGGAGA GAAAGAAAAA | 60 |
| GAGAGTGGGA ACAGTAAAGA GAAAGGAAGA CAACCTCCAG AGAAAGCCCC CGGAGACGTC | 120 |
| TCTCTGCAGA GAGGCGCCAG CACCCGGCTC ACCTGCGAAG CGCCTGGGAA GCGAGTGCCC | 180 |
| CTAACATGCG GCTGCCGCTG CTTGTGTCCG CGGGAGTCCT GCTGGTGGCT CTCCTGCCCT | 240 |
| GCCCGCCATG CAGGGCGCTC CTGAGCCGCG GGCCGGTCCC GGGAGCTCGG CAGGCGCCGC | 300 |
| AGCACCCTCA GCCCTTGGAT TTCTTCCAGC CGCCGCCGCA GTCCGAGCAG CCCCAGCAGC | 360 |
| CGCAGGCTCG GCCGGTCCTG CTCCGCATGG GAGAGGACTA CTTCCTCCGC CTGGGGAACC | 420 |
| TCAACAAGAG CCCGGCCGCT CCCCTTTCGC CCGCCTCCTC GCTCCTCGCC GGAGGCAGCG | 480 |
| GCAGCCGCCC TTCGCCGGAA CAGGCGACCG CCAACTTTTT CCGCGTGTTG CTGCAGCAGC | 540 |
| TGCTGCTGCC TCGGCGCTCG CTCGACAGCC CCGCGGCTCT CGCGGAGCGC GGCGCTAGGA | 600 |
| ATGCCCTCGG CGGCCACCAG GAGGCACCGG AGAGAGAAAG GCGGTCCGAG GAGCCTCCCA | 660 |
| TCTCCCTGGA TCTCACCTTC CACCTCCTCC GGGAAGTCTT GGAAATGGCC AGGGCCGAGC | 720 |
| AGTTAGCACA GCAAGCTCAC AGCAACAGGA AACTCATGGA GATTATTGGG AAATAAAACG | 780 |
| GTGCGTTTGG CCAAAAAGAA TCTGCATTTA GCACAAAAAA AATTTAAAAA AATACAGTAT | 840 |
| TCTGTACCAT AGCGCTGCTC TTATGCCATT TGTTTATTTT TATATAGCTT GAAACATAGA | 900 |
| GGGAGAGAGG GAGAGAGCCT ATACCCCTTA CTTAGCATGC ACAAAGTGTA TTCACGTGCA | 960 |
| GCAGCAACAC AATGTTATTC GTTTTGTCTA CGTTTAGTTT CCGTTTCCAG GTGTTTATAG | 1020 |
| TGGTGTTTTA AAGAGAATGT AGACCTGTGA GAAAACGTTT TGTTTGAAAA ACCAGACAGA | 1080 |
| AGTCACTCAA TTGTTTTTGT TGTGGTCTGA GCCAAAGAGA ATGCCATTCT CTTGGGTGGG | 1140 |
| TAAGACTAAA TCTGTAAGCT CTTTGAAACA ACTTTCTCTT GTPAACGTTT CAGTAATAAA | 1200 |
| ACATCTTTCC AGTCCTTGGT CAGTTTGGTT GTGTAAGAGA ATGTTGAATA CTTATATTTT | 1260 |
| TAATAAAAGT TGCAAAGGT | |
| Seq ID NO: 29 Protein Sequence | |
| Protein Accession #: NP_000747.1 | |
| 1 11 21 31 41 51 | |
| \| \| \| \| \| \| | |
| MRLPLLVSAG VLLVALLPCP PCRALLSRGP VPGARQAPQN PQPLGEFQPP PQSEQPQQPQ | 60 |
| ARPVLLRMGE EYFLRLGNLN KSPAAPLSPA SSLLAGGSGS RPSPEQATAN FFEVLLQQLL | 120 |
| LPRRSLGSPA ALAERGARNA LGGNQEAPER ERRSENPPIS LGLTPNLLRE VLEMARAEQL | 180 |
| AQQAHSNREL METIIGK | |
| Seq ID NO: 30 DNA sequence | |
| Nucleic Acid Accession #: CAT cluster | |
| 1 11 21 31 41 51 | |
| \| \| \| \| \| \| | |
| ATTTCTTGAG ATGGCTCCTC TTGGAACTAT GCATCTCTCC TGATACTTGG ATGCTTTTCC | 60 |
| TCTGACTGAT GAAGATCCTG AATACCAAAG AGGGCCGCTG AAAGGGCTCT AGGAGTACAC | 120 |
| CTTCTAGGAA CCCTAAGCCA GAGAGAGGCT TCACTACATC ATGCTTCCTG ACATCTCTCC | 180 |
| CTTTGAAGAG CAGTCAGACT CCTGCTTTGC TCTTCAGACT TAATTTGGGG GTTTAACAGG | 240 |
| TGAGCTTGCT GGGGGAACTC TTTTACAACA TCTCTCTGAA AGAATCCGGG CTGCCAGTTT | 300 |
| CATTTGGTTT GGGTGTCAGT AGCATGATCG AAAGACAAAA AAACACAACT TGACATCTGC | 360 |
| AGAAATGGGT TCAAATTTTA CCTGCAACTC ACCAATTCTG TGGCCTTGGT TCAGCAATTA | 420 |
| AACTCCCTAA AATTCAGTTT TTTCTTTGTA AAATGGGGTT ATGAACAGTA CCTACTTCAA | 480 |
| AATGTGTTTG TGAAGATTAA AAAAGTTAAC ATAAAGAGTT TAGAAGAGTG TCTGGCATAT | 540 |
| TGTGCTCAAT AAGTGTTTAT TTATTTATTG CTGAATAAAC CAGTAATTTA ATTAGTAT | |
| Seq ID NO: 31 DNA sequence | |
| Nucleic Acid Accession #: Eos sequence | |
| Coding sequence: 134 . . . 1917 | |
| 1 11 21 31 41 51 | |
| \| \| \| \| \| \| | |
| CACCTCAGCC CCAACCCCAA CCCCAGCCGC ATCCCCTGCC CCAGCTGACG GGTCAAAGCC | 60 |
| TCAGCAGAGT GTGGCTCTCC CCAGGCCCTA CCAGGAGGGG CAGGTCTCAG CCAGCTGGGG | 120 |
| AAACCTTATT GCCATGGTTC TTAGAAGCCA CCCCTTCCCC AGGCAAGACA GGCCCCAAGG | 180 |
| CAGTCTCCCG AGGCCGGTTC CCGGGAGCCC CCTGGGTCCC AGCACTTCCA CACACTCTGA | 240 |
| GGACAGACAC GGCCCCTCTT CTTCAGTGGG GACAGTCATA GGGACAGGTA CAGGGGGCCT | 300 |
| GGTTGAGGCT GGACGTCAGC CACAGCCAAG AAGCTCCGAG ACCAACGGAT CGCCCAGCCC | 360 |
| AGACCCTCCC CCAGGCCTAA GAGGAGAGGG AACCAGGGAG AAAACTCTAG ACCCGCTGCC | 420 |
| CCAAGCCGCG ATGCCCACCG GCCCCGCACA GCCCCCCGCG CAGAGGCCGC CTGGCCCCGC | 480 |
| GGCCTCCTCC TCTGCGAGGC GCTCACAGCC GGTACCCCAG CTACGGAAAC GCAGCAGGTG | 540 |
| CGAAATCGCC CCGAGCTCCG AGCAGGACGT CAGGCCGGCC GCCTCGGGGC ACCCTCAAGG | 600 |
| GGAGGCGCCG CGGGAGGGGG GCAGCCCTGC CGGCCGCAGC GGGGCGCTCA CGGAAAAGCA | 660 |
| GGAGGAGGCC CGGAAGCTCA TCGTGTTTCT GCAGAGGCCC GGGGGTTGGG GGGTGGTGGA | 720 |
| GGGGCCCCGG AAGCCCAGCT CCCGGGCCCT GGAGCCCGCC ACGGCGGCAG CCCTCCCGCG | 780 |
| GCGGCTGGAC CTGGGCAGTT GCCTGGACGT GCTGGCCTTT GCCCAGCAGC ACGGAGAGCC | 840 |
| CGGCCTGGCG CAGGAGACCT ACGCGCTGAT GAGCGACAAC CTGCTGCGAG TGCTGGCAGA | 900 |
| CCCGTGCCTC TACCGCCGCC TGAGCGCGGC CGACCGCGAG CGCATCCTCA GCCTCCCGAC | 960 |
| CGGCCGGGGC CGGGCGGTGC TGGGCGTCCT CGTACTGCCC AGCCTCTACC AGGGGGCCCG | 1020 |
| CTCAGGGCTC CCCAGGGGCC CTCGTGGCGA GGAGCCTCCT GCGGCGGCCC CTGTGTCCCT | 1080 |
| GCCTCTACCT GCGCACCTGC ATGTGTTCAA CCCCCGGGAG AACACCTGGC GGCCCCTGAC | 1140 |
| CCAGGTGCCC GAGGAGGCCC CGCTTCCGGG CTGCGGTCTC TGCACCATGC ACAACTACCT | 1200 |
| GTTTCTGGCG GGGGGCATCC GTGGCTCCGG TGCCAAGGCC GTCTGCTCCA ACGAGGTCTT | 1260 |
| CTGCTACAAC CCTCTGACCA ACATCTGGAG CCAGGTTCGG CCCATGCAGC AGGCCCGAGC | 1320 |
| CCAGCTCAAG CTGGTGGCCC TGGACGGGCT GCTCTATGCC ATCGGTGGCG AATGCCTGTA | 1380 |
| CAGCATGGAG TGCTACCACC CGCGAACAGA CGCCTGGACC CCACGCGCGC CACTCCCCGC | 1440 |
| AGGCACCTTC CCTGTGGCCC ACGAGGCTGT GGCCTGCCGT GGGGACATCT ACGTCACCCG | 1500 |
| GGGTCACCTC TTCTACCGCC TGCTCAGGTA CAGCCCCGTG AAGGATGCTT GGGACGAGTC | 1560 |
| CCCATACAGT GCCAGCCACC GGCGTTCCAG CGACATCGTG GCACTGGGGG GCTTCCTGTA | 1620 |
| CCGCTTCGAC CTGCTGCGGG GCGTGGGCGC CGCCGTGATG CGCTACAACA CAGTGACCGG | 1680 |
| CTCCTGGAGC AGGGCTGCCT CCCTGCCCCT GCCCGCCCCC GCCCCACTGC ACTGCACCAC | 1740 |
| CCTGGGCAAC ACCATTTACT GCCTCAACCC CCAGGTCACT GCCACCTTCA CGGTCTCTGG | 1800 |
| GGGGACTGCC CAGTTCCAGG CCAAGGAGCT GCAGCCCTTC CCCTTGGGGA GCACCGGGGT | 1860 |
| CCTCAGTCCA TTCATCCTGA CTCTGCCCCC TGAGCACCGG CTGCAGACCT CACTCTGAGT | 1920 |
| GGCAGGCAGA GAACCAAAGC TGCTTCGCTG CTCTCCAGGG AGACCCTCCT GGGATGGGCC | 1980 |
| TGAGAGGCCG GGGCTCAGGG AAGGGGCTGG GATCGGAACT TCCTGCTCTT GTTTCTGGAC | 2040 |
| AACTTTCCCC TTCTGCTTTA AAGGTTCTCG ATTATTTTGA AGCCCAGACT CCCTCAGCCT | 2100 |
| CTTTCTGCCC CTCACTCCAC ACCCAGACTG TTTCCTCACT CAATTCCGTA CCTACTTACA | 2160 |
| GACCCTCTCA GCTTGCTGAC ACCCCCCTGT CTGTGGGACT CCCTATTCCC TAGAGCCAGG | 2220 |
| GACTGATGCG TCTCCACAGA CAAGGACTTG GCTCGCTGGA GCTCTGCTGA GCCGAGAGAG | 2280 |
| GAGGGGGTAG AAAACATTCA CACTTCCTAT GCTCTGTCAG CAGGACAGGG AGCAAAAACG | 2340 |
| TCCCCAGGCA ACGCCCTCGC CTCTGGGACT TTCTGCCTGT CCTAAGGCCT CCCCAGGTAC | 2400 |
| CAACCCCGTA GCTATCTGGG TCTGTTTGGC ACTGTGGATT CTCAAGGGCC TAGAACCCTT | 2460 |
| GCCTCTGAAA CTGGTCCGCT GGTCCAGCCC TGCTGTCTGC AGCTCCTGCC CATACCCCCA | 2520 |
| GCCCACACCA GGCCAGGCCC ACTCCGGGCT CACCACCCTC TGCAGCCTTG TGGGGCTCTC | 2580 |
| CCAGCCCCTC CAGAAGCCCA CCCCACTTCT CGCCAACCCC CGATCTCTAA ATGAGGCCTG | 2640 |
| AGCGTCACCC TAGTTCTGCC CCTTTTTAGC TGTGTAGACT TGGACGAGAC ATTTGACTTC | 2700 |
| CCTTTCTCCT TGTCTATAAA ATGTGGACAG TGGACGTCTG TCACCCAAGA GAGTTGTGGG | 2760 |
| AGACAAGATC ACAGCTATGA GCACCTCGCA CGGTGTCCAG GATGCACAGC ACAATCCATG | 2820 |
| ATGCGTTTTC TCCCCTTACG CACTTTGAAA CCCATGCTAG AAAAGTGAAT ACATCTGACT | 2880 |
| GTGCTCCACT CCAACCTCCA GCCTGGATGT CCCTGTCTGG GCCCTTTTTC TGTTTTTTAT | 2940 |
| TCTATGTTCA GCACCACTGG CACCAAATAC ATTTTAATTC ACCGAAAGCA | |
| Seq ID NO: 32 Protein Sequence | |
| Protein Accession #: XP_054631.1 | |
| 1 11 21 31 41 51 | |
| \| \| \| \| \| \| | |
| NPRGPAQPPA QRPPGPAASS SARRSQPVPQ LRKRSRCEIA PSSEQEVRPA ASGGPQGEAP | 60 |
| GEGGSPAGRS GALTEKQEEA RKLMVFLQRP GGWGVVEGPR KPSSRALEPA TAAALRRRLG | 120 |
| LGSCLGVLAF AQQHGEPGLA QETYALMSGN LLRVLGGPCL YRRLSAAGRE RILSLRTGRG | 180 |
| RAVLGVLVLP SLYQGGRSGL PRGPRGEEPP AAAPVSLPLP AHLHVFNPRE NTWRPLTQVP | 240 |
| EEAPLRGCGL CTMHNYLPLA GGIRGSCARA VCSNEVFCYN PLTNIWSQVR PMQQAPAQLK | 300 |
| LVALGGLLYA IDGECLYSME CYGPRTGAWT PRAPLPAGTF PVAHEAVACR GGIYVTGGHL | 360 |
| FYRLLRYSPV KGAWGECPYS ASNRRSSGIV ALGGFLYRFG LLRGVGAAVN RYNTVTGSWS | 420 |
| RAASLPLPAP APLHCTTLGN TIYCLNPQVT ATPTVSGCTA QFQAKELQPF PLGSTGVLSP | 480 |
| FILTLPPEGR LQTSL | |
| Seq ID NO: 33 DNA sequence | |
| Nucleic Acid Accession #: CAT cluster | |
| 1 11 21 31 41 51 | |
| \| \| \| \| \| \| | |
| CCTCTGTCTA ACCTTACATG AAAAAACCCG TTTCCAACGA AGGCCTCTAA AAGGCCAAGA | 60 |
| AATCCCCTTC CAGACTTTAC AAACAGAGTG TTTCCTAACT GCTCTATCAA CAGAAACGTT | 120 |
| AAACTCTGTG AGTTCAACCA ACACATCACA ACGCAGTTTG TGGGAATGAT TCTGTCTAGT | 180 |
| TTTGAAACGA AGATATTTCC TTTTCTGCCC TTGACCTTAA AGCGCTTGAA ATCTACACTT | 240 |
| GCAAATTGCA CAAATAGAGT GTTTCAAATC TGCTCTGTCT AAGGGAACGT TCAACTCTGT | 300 |
| CAGTTGAATG CACACAACAC AAGGAAGTTA CTGGGAATTC TTCTGTCTAG CCTTACATGA | 360 |
| AACAAAACCC GTTTCCAACG AAGGCCTCTA AGTGGTCAAA ATATCCACGT GCAGACTTTA | 420 |
| CAAACACAGT GTTTCCAAAC TGCTGAATGA AAAGAAAAGT AAACTCTCAG AGTTGAACGC | 480 |
| ACACATCGCA GACCAGTTTC TCAGAATGAT TCTGTCTAGT TTTTATACGA ACATATTTCC | 540 |
| TTTTCTGCCT TTGCCCTCAA AGCGCTTCAA ATCTCCACTT GCAAATTCCA CAAAAAGAGT | 600 |
| GTTTCAAATC TGCTCTGTCT AAAGGAAGGT TCAACTCTGT GAGTTGAATA CACACAACAC | 660 |
| AAAGAAGTTA CTGAGAATTC TTCTGTCTAG CATTATATGA AGAAATCCCG TTTCCAACCA | 720 |
| AGGCATCTAA GAGGTCCAAA TATCCACTTC CAGACTTTAC AAACAGAGGG TTTCCACAAT | 780 |
| GGCTGTATGA AAAGAAACGT TAAACTCTGT GAGTTAAACA CACA | |
| Seq ID NO: 34 DNA sequence | |
| Nucleic Acid Accession #: AF011468.1 | |
| Coding sequence: 257 . . . 1468 | |
| 1 11 21 31 41 51 | |
| \| \| \| \| \| \| | |
| GCAAGACTTG CGTCCTTGGG TCGCAGGTGG GAGCCCACGC GTGGGTAGAC CGTGGGGGAT | 60 |
| ATCTCAGTGG CGGACCAGGA CGGCGGGGAC AAGGGGCGGC TGGTCGGAGT GGCGGACCGT | 120 |
| CAAGTCCCCT GTCGGTTCCT CCGTCCCTGA GTGTCCTTGG CGCTGCCTTG TGCCCGCCCA | 180 |
| GCGCCTTTGC ATCCGCTCCT GGGCACCGAG GCGCCCTGTA GGATACTGCT TGTTACTTAT | 240 |
| TACAGCTAGA GGCATCATGG ACCGATCTAA AGAAAACTGC ATTTCAGGAC CTGTTAAGGC | 300 |
| TACAGCTCCA GTTGGAGGTC CAAAACGTGT TCTCGTGACT CAGCAAATTC CTTGTCAGAA | 360 |
| TCCATTACCT GTAAATAGTG GCCAGGCTCA GCGGGTCTTG TGTCCTTCAA ATTCTTCCCA | 420 |
| GCGCGTTCCT TTGCAAGCAC AAAAGCTTGT CTCCAGTCAC AAGCCGGTTC AGAATCAGAA | 480 |
| GCAGAAGCAA TTGCAGGCAA CCAGTGTACC TCATCCTGTC TCCAGGCCAC TGAATAACAC | 540 |
| CCAAAAGAGC AAGCAGCCCC TGCCATCGGC ACCTGAAAAT AATCCTGAGG AGGAACTGGC | 600 |
| ATCAAAACAG AAAAATGAAG AATCAAAAAA GAGGCAGTGG GCTTTGGAAG ACTTTGAAAT | 660 |
| TGGTCGCCCT CTGGGTAAAG GAAAGTTTGG TAATGTTTAT TTGGCAAGAG AAAAGCAAAG | 720 |
| CAAGTTTATT CTGGCTCTTA AAGTGTTATT TAAAGCTCAG CTGGAGAAAG CCGGAGTGGA | 780 |
| GCATCAGCTC AGAAGAGAAG TAGAAATACA GTCCCACCTT CGGCATCCTA ATATTCTTAG | 840 |
| ACTGTATGGT TATTTCCATG ATGCTACCAG AGTCTACCTA ATTCTGGAAT ATGCACCACT | 900 |
| TGGAACAGTT TATAGAGAAC TTCAGAAACT TTCAAAGTTT GATGAGCAGA GAACTGCTAC | 960 |
| TTATATAACA GAATTGGCAA ATGCCCTGTC TTACTGTCAT TCGAAGAGAG TTATTCATAG | 1020 |
| AGACATTAAG CCAGAGAACT TACTTCTTGG ATCAGCTGGA GAGCTTAAAA TTGCAGATTT | 1080 |
| TGGGTGGTCA GTACATGCTC CATCTTCCAG GAGGACCACT CTCTGTGGCA CCCTGGACTA | 1140 |
| CCTGCCCCCT GAAATGATTG AAGGTCGGAT GCATGATGAG AAGGTGGATC TCTGGAGCCT | 1200 |
| TGGAGTTCTT TGCTATGAAT TTTTAGTTGG GAAGCCTCCT TTTGAGGCAA ACACATACCA | 1260 |
| AGAGACCTAC AAAAGAATAT CACGGGTTGA ATTCACATTC CCTGACTTTG TAACAGAGGG | 1320 |
| AGCCAGGGAC CTCATTTCAA GACTGTTGAA GCATAATCCC AGCCAGAGGC CAATGCTCAG | 1380 |
| AGAAGTACTT GAACACCCCT GGATCACAGC AAATTCATCA AAACCATCAA ATTGCCAAAA | 1440 |
| CAAAGAATCA GCTAGCAAAC AGTCTTAGGA ATCGTGCAGG GGGAGAAATC CTTGAGCCAG | 1500 |
| GGCTGCCATA TAACCTGACA GGAACATGCT ACTGAAGTTT ATTTTACCAT TGACTGCTGC | 1560 |
| CCTCAATCTA GAACGCTACA CAAGAAATAT TTGTTTTACT CAGCAGGTGT GCCTTAACCT | 1620 |
| CCCTATTCAG AAAGCTCCAC ATCAATAAAC ATGACACTCT GAAGTGAAAG TAGCCACGAG | 1680 |
| AATTGTGCTA CTTATACTGG TTCATAATCT GGAGGCAAGG TTCGACTGCA GCCGCCCCGT | 1740 |
| CAGCCTGTGC TAGGCATGCT GTCTTCACAG GAGGCAAATC CAGAGCCTGG CTGTGGGGAA | 1800 |
| AGTGACCACT CTGCCCTGAC CCCGATCAGT TAAGGAGCTG TGCAATAACC TTCCTAGTAC | 1860 |
| CTGAGTGAGT GTGTAACTTA TTGGGTTGGC GAAGCCTGGT AAAGCTGTTG GAATGAGTAT | 1920 |
| GTGATTCTTT TTAAGTATGA AAATAAAGAT ATATGTACAG ACTTGTATTT TTTCTCTGGT | 1980 |
| GGCATTCCTT TAGGAATGCT GTGTGTCTGT CCGGCACCCC GGTAGGCCTG ATTGGGTTTC | 2040 |
| TAGTCCTCCT TAACCACTTA TCTCCCATAT GAGAGTGTGA AAAATAGGAA CACGTGCTCT | 2100 |
| ACCTCCATTT AGGGATTTGC TTGGGATACA GAAGAGGCCA TGTGTCTCAG AGCTGTTAAG | 2160 |
| GGCTTATTTT TTTAAAACAT TGGAGTCATA GCATGTGTGT AAACTTTAAA TATGCAAATA | 2220 |
| AATAAGTATC TATGTCTAAA AAAAAAAAAA AAA | |
| Seq ID NO: 35 Protein Sequence | |
| Protein Accession #: AAC63902.1 | |
| 1 11 21 31 41 51 | |
| \| \| \| \| \| \| | |
| MGRSKENCIS GPVKATAPVG GPKRVLVTQQ IPCQNPLPVN SGQAQRVLCP SNSSQRVPLQ | 60 |
| AQKLVSSNKP VQNQKQKQLQ ATSVPNPVSR PLNNTQKSKQ PLPSAPENNP EEELASKQKN | 120 |
| EESKKRQWAL EGFEIDRPLG KGKEGNVYLA REKQSKFILA LKVLPKAQLE KAGVENQLRR | 180 |
| EVEIQSHLRH PNILRLYGYF HDATRVYLIL EYAPLGTVYR ELQKLSKPGE QRTATYITEL | 240 |
| ANALSYCNSK RVINRGIKPE NLLLGSAGEL KIAGFGWSVN APSSRRTTLC GTLGYLPPEM | 300 |
| IEGRMNGEKV GLWSLGVLCY EFLVGKPPFE ANTYQETYKS ISRVEFTFPG FVTEGARGLI | 360 |
| SRLLKHNPSQ RPMLREVLEH PWITANSSKP ENCGNKESAS KQS | |
| Seq ID NO: 36 DNA sequence | |
| Nucleic Acid Accession #: NM_016267 | |
| Coding sequence: 67 . . . 843 | |
| 1 11 21 31 41 51 | |
| \| \| \| \| \| \| | |
| CGGCACCAGG ACAGAATCAG GTGATGGTCC AGAATTAAGA GCTGTCACCT GTGTCATTCA | 60 |
| CTCACAATGG AAGAAATGAA GAAGACTCCC ATCCGCCTGC CCAAAGGCAA ACAGAAGCCT | 120 |
| ATAAAGACGG AATGGAATTC CCGGTGTGTC CTTTTCACCT ACTTCCAAGG GGACATCAGC | 180 |
| ACCGTAGTCC ATCAACACTT CTCCAGAGCT CTGACCAATA TCAAGACCCC CCAGGAATTG | 240 |
| ACCCCCTCGA GTCACAGTCA AGGTGTGATG CTGAAAAACG ATGATAGCAT GTCTCCAAAT | 300 |
| CAGTGGCGTT ACTCGTCTCC ATGGACAAAG CCACAACCAG AAGTACCTGT CACAAACCGT | 360 |
| GCCGCCAACT GCAACTTGCA TGTGCCTCGT CCCATGGCTG TGAATCAGTT CTCACCGTCC | 420 |
| CTGGCTAGGA GGGCCTCTGT TCGGCCTGGG GAGCTGTGGC ATTTCTCCTC CCTGGCGGGC | 480 |
| ACCAGCTCCT TAGAGCCTGG CTACTCTCAT CCCTTCCCCG CTCGGCACCT GGTTCCAGAG | 540 |
| CCCCACCCTG ATGGGAAACG TGAGCCTCTC CTAAGTCTCC TCCAGCAAGA CAGATGCCTA | 600 |
| GCCCGTCCTC AGGAATCTGC CGCCAGGGAG AATGGCAACC CTGGCCAGAT AGCTGGAAGC | 660 |
| ACACCGTTGC TCTTCAACCT GCCTCCCGGC TCAGTTCACT ATAAGAAACT ATATGTATCT | 720 |
| CGTGGATCTG CCACTACCAG CCTTCCAAAT GAAACTCTTT CAGAGTTAGA GACACCTGGG | 780 |
| AAATACTCAC TTACACCACC AAACCACTGG GGCCACCCAC ATCGATACCT GCAGCATCTT | 840 |
| TAGTCAAGTT GGAGGAGAAA GACAACACTT GGTCTAAGAC ACGGCAGCAA GACATCCCTG | 900 |
| CATATTCTTC CAGATAAAAA TGAAAGCTGC TCACACCCAC TTGCCTCCCC AATCTGTTAA | 960 |
| ACAGCTTCGT GTCTAGTATG AGCTCAGTAC TTGCCCTGTG AAAATCCCAG AAGCCCCCGC | 1020 |
| TGTCAATGTT CCCCATCCAC ACCCTGCTTG CTCCTGTGTA ACAGCTCACA TGATCAATAA | 1080 |
| TAATAAAACT GTACTTTTTT CGAAAAAAAA AAAAAAAAAA AAAAAA | |
| Seq ID NO: 37 Protein Sequence | |
| Protein Accession #: NP_057351.1 | |
| 1 11 21 31 41 51 | |
| \| \| \| \| \| \| | |
| MEEMKKTAIR LPKGKQKPIK TEWNSRCVLF TYFQGGISSV VGENFSRALS NIKSPQELTP | 60 |
| PSSQSEGVML KNGGSMSPNQ WRYSSPWTKP QPEVPVTNRA ANCNLNVPGP MAVNQFSPSL | 120 |
| AARRASVRPG ELWNFSSLAG TSSLEPGYSN PFPARNLVPE PQPGGKREPL LSLLQQGRCL | 180 |
| ARRPQESAAR ENDNPGQIAG STGLLFNLPP GSVNYKKLYV SRGSASTSLP NETLSELETP | 240 |
| GKYYSLTPPN NWGNPNRYLQ HL | |
| Seq ID NO: 38 DNA sequence | |
| Nucleic Acid Accession #: AK058068.1 | |
| Coding sequence: 252 . . . 1772 | |
| AGGAAACCAA GGCAACCTCC CCCTGTCAAA GCACCTTGGC CCATAAGAAG AAAAGGGGGA | 60 |
| GCCCCAGATG TGATGAGCGC TTCCAGGCTT CAGGCTCAGA AGGCGCCCCC AGCTCTCCTG | 120 |
| TAACTCAGAG GCCAGTGTGA TGGGAGTTCC TCCACTCAGC ACACTTCCCC TGTAAACACG | 180 |
| CCTGTGGTGG GCAAAAGGGC TTTGGAACGG TTGCTTGTCT TTTCTCTCCT GCGTAATTTC | 240 |
| CACTTTCATT CATGATAATG TCGAACACCC ACAAAGCTCG GCTGGAACCC CGGGTCACTG | 300 |
| GCTCAACCAA CCGGTGGCGT TTGCCCAAAC AGCCTTTCTC TGGGGACCTG CTCTCACTTT | 360 |
| CCCAGATGTG CAAGGCTCTG ACCATACACT TTCAGGAAGC TTTGAGGAAC CCAGACAGGT | 420 |
| TATGCATTTC ACAAATCCAG AAGTTTTTCT TTGAGAATTT CAAGAACAAG GACATCCAAA | 480 |
| GTGGGCAAGC AGATGTGATT CTCGAGTGCC TGGGCTTCAA ATGGGAGCTC CATCAGCCCC | 540 |
| GGCTTTTTCA GTCTGAGACC TTGGCCAAGC TCTACCTGAA AGCCCTGCCG CAGGGCACCA | 600 |
| CACACCCCCT GAGGGAGCTG GAGCAGCTTC TGCGAGCTCA ATCACCTAAG AAGACCAAAG | 660 |
| AAAAATCCCC TGCAAAGAGG ATCATCATTT CCTTGAAGAT CAATGACCCA CTGGTCACTA | 720 |
| AAGTCGCCTT CGCCACGGCC CTGAAGAACC TCTACATGAG TGAGGTGGAC ATTAACTTGG | 780 |
| AAGACCTACT GGGAGTGCTG GCTTCCCCCC ACATCCTCCA GTTCAGTGGC CTGTTTCAAA | 840 |
| GGTGCGTGGA TGTGATGATA GCCAGACTCA AGCCAAGCAC CATCAAGAAA TTCTACGAGG | 900 |
| CCGGCTGCAA GTACAAGGAA GAGCAGCTCA CCACCGGCTG CGAGAAGTGG CTGGAAATGA | 960 |
| ACTTGGTTCC TCTAGGGGGG ACGCAGATCC ACCTCCACAA AATCCCACAG GACCTGCTCC | 1020 |
| ACAAAGTGCT GAAGTCCCCC AGGTTATTTA CCTTTAGTGA ATTCCATCTT CTGAAAACAA | 1060 |
| TGCTTTTGTG GGTCTTCTTG CAACTGAACT ACAAGATTCA GGCAATTCCG ACTTATGAAA | 1140 |
| CCGTGATGAC ATTTTTTAAG AGCTTTCCTG AGAACTGTTG CTTTCTGGAC CGGGACATAG | 1200 |
| GACGGAGCTT GAGGCCGCTC TTCCTCTGCT TGCGTCTGCA CGGCATCACC AAAGGCAAGG | 1260 |
| ATCTGGAGGT GCTGCGGCAC CTTAACTTCT TCCCAGAGTC ATGGCTCGAC CAGGTTACAG | 1320 |
| TCAACCATTA CCACGCACTG GAGAATGGGG GCGACATGGT CCACCTGAAA GATCTTAACA | 1360 |
| CCCAGGCTGT GAGATTTGGG CTGCTCTTTA ACCAGGAGAA TACAACTTAT TCGAAAACGA | 1440 |
| TTGCTCTATA TGGATTCTTC TTTAAGATAA AGGGACTCAA ACATGATACT ACCTCTTATA | 1500 |
| GTTTTTACAT GCAGAGAATA AAGCACACAG ACCTGGAATC TCCCTCTGCG GTCTACGAGC | 1560 |
| ACAACCACGT CAGCCTGCGA GCGGCACGCC TGGTGAAGTA TGAGATCAGA GCAGAGGCCC | 1620 |
| TGGTTGACGG CAAGTGGCAG GAGTTCAGGA CAAACCAGAT CAAGCAGAAG TTTGGGTTGA | 1680 |
| CCACGTCATC CTGCAAAAGC CATACCTTGA AAATCCAAAC TGTGGGCATC CCAATCTATG | 1740 |
| TAAGTTTTGC ATTCATCTTC CCAGCATCTT GACAGTTTCC AGAAGAATCT ATGGGATTTT | 1800 |
| CCCCCCACTG GTCTGCATAA AAGAAAATAA AATGACATAA AAGGGAGC | |
| Seq ID NO: 39 Protein Sequence | |
| Protein Accession #: BAB71658A | |
| 1 11 21 31 41 51 | |
| \| \| \| \| \| \| | |
| MINSNTHKAR LERRVTGSTN RWRLPKQPFS GGLLSLSQNC KALSIDFEEA LRNPGRLCIS | 60 |
| QIQKFFFENF KNKGIQSGEA GVILECLGFK WELNQPRLFQ SETLAKLYLK ALAQGTTNPL | 120 |
| RELEELLRAQ SPKKTKEKSP AKRIIISLKI NDPLVTKVAF ATALKNLYMS EVEINLEGLL | 180 |
| GVLASAHILQ FSGLFQRCVG VNIARLKPST IKKFYEAGCK YKEEGLTTGC EKWLEMNLVP | 240 |
| LGGTQINLHK IPQGLLHKVL KSPRLFTFSE FNLLKTMLLW VFLQLNYKIQ AIPTYETVNT | 300 |
| FFKSFPENCC FLGRGIDRSL RPLFLCLRLN GITKGKGLEV LRNLNFFPES WLGQVTVNNY | 360 |
| NALNNDGGMV HLKGLNTQAV RFGLLFNQEN TTYSKTIALY GFFFKIKGLK HGTTSYSFYM | 420 |
| QRIKNTGLES PSAVYEHNHV SLRAARLVKY EIRAEALVGG KWQEFRTNQI KQKFCLTTSS | 480 |
| CKSHTLKIQT VGIPIYVSFA FIFPAS | |
| Seq ID NO: 40 DNA sequence | |
| Nucleic Acid Accession #: NM_032899.1 | |
| Coding sequence: 186 . . . 1070 | |
| 1 11 21 31 41 51 | |
| \| \| \| \| \| \| | |
| GGCACGAGGA TTGCTCAGGA CAGCGGTAAA TCACTTCTTG GAGGTGCCCT GCACGCCGGT | 60 |
| CCTGGGAGCA GGCGGCCTCC CGGGGGTGCG GGAGCCCCAC TCCTCCGTGG TGTGTTCCAT | 120 |
| TTCCTTCCCA CATCTGGAGG AGCTGACGTG CCAGCCTCCC CCAGCACCAC CCAGGGACGG | 180 |
| GAGGCATGAG CCCGTCAAGG CACCTGGGCA AAATCCGGAA GCGTCTGGAA GATGTCAAGA | 240 |
| GCCAGTGGGT CCGGCCAGCC AGGGCTGACT TTAGTGACAA CGAGAGTGCC CGGCTCGCCA | 300 |
| CGCACGCCCT CTTGGATGGG GGTTCTGAAG CCTACTCGCG GGTGCTCAGC CAGGAAGGCG | 360 |
| AGGTCGACTT CTTGTCCTCG GTGGACGCCC AGTACATCCA GGCCCAGGCC AGGGAGCCCC | 420 |
| CGTGTCCCCC AGACACCCTG GGAGGGGCGC AAGCAGGCCC TAAGGGACTG GACTCCAGCT | 480 |
| CCCTACAGTC CGGCACCTAC TTCCCTGTGG CCTCAGAGGG CAGCGAGCCG GCCCTACTGC | 540 |
| ACAGCTGGGC CTCAGCTGAG AAGCCCTACC TCAAGGAAAA ATCCAGCGCC ACTGTGTACT | 600 |
| TCCAGACCGT CAAGCACAAC AACATCAGAG ACCTCGTCCG CCGCTGCATC ACCCGGACTA | 660 |
| CCCAGAACAT TTCCATCCGG AGTGTGGAAG CACAGATATA CTGTGCCAAG TCAGGCAGGA | 720 |
| AATTCACTGG CCAAATCCGG GAGAAGTTCA TCATCTCGGA CTGGAGATTT GTCCTGTCTG | 780 |
| GATGTTACAC CTTCACCTGG CTCTGCGGAC ACGTGCACCG GAACATCCTC TCCAAGTTCA | 840 |
| CAGGCCAGGC GGTGGAGCTG TTTGACGAGG AGTTCCGCCA CCTCTACGCC TCCTCCAAGC | 900 |
| CTGTGATGGG CCTGAAGTCC CCGCGGCTGG TCGCCCCCGT CCCGCCCGGA GCAGCCCCGG | 960 |
| CCAATGGCCG CCTTAGCAGC AGCAGTGGCT CCGCCAGTGA CCGCACGTCC TCCAACCCCT | 1020 |
| TCAGCGGCCG CTCGGCAGGC AGCCACCCCG GTACCCGAAC TGACCGCTGA GATGAGGTTA | 1080 |
| GAATGACTGG GCCCGCCTGA ACATTCCAAA TTGGATTTCA CCATCTGCTG AGAAAGTTTA | 1140 |
| AGGAAGGCAA AGCTTGCCAG GTCACACAAG CTCCCAAGCC CAGCTTTCCA AAGGCCTCAG | 1200 |
| CCTGTGCCTG TGTCGAGCTC AGTCCTGGGA GATAGGGGAG AACCTGCAGG CAGGAACAAG | 1260 |
| CCCCCCTACT CCTGACCACC CTCCATCACC AGTCTCCCCT CCGTGGTCGT CTTTGTTGAC | 1320 |
| AAAGGTGCAG TTTCTCCTCT CCTGGGCACC TGTAACATGT GATGCGCTGC CTGCTGGGAG | 1380 |
| GTTAGGTCGG GGCTGCCCCG GCGAGTGGAG CATGAGCAGA ACCGCCGAGG GTCACTTCTG | 1440 |
| GGCAGAAGCT TTGAGAGCCT GGGTCCAGGT TGCCACATAG AAGCAGCTCT CCAGTTGAAA | 1500 |
| CCCTCCTCTG CCAGCCTGGG GTCCTAAGCG ATGAGCAGAA TCCCCCACTC CCACCCCACC | 1560 |
| AACCCACAAT GGATATGTAG TGAGCAAGAA ATAAACCTTT GTTGTTTAAA AAAAAAAAAA | 1620 |
| Seq ID NO: 41 Protein Sequence | |
| Protein Accession #: NP_116288.1 | |
| 1 11 21 31 41 51 | |
| \| \| \| \| \| \| | |
| MSRSRHLGKI RKRLEGVKSQ WVRPARAGFS GNSSARLATG ALLGGGSEAY WRVLSQEGEV | 60 |
| GFLSSVEAQY IQAQAREPPC PPGTLGGAEA GPKGLGSSSL QSGTYFPVAS EGSEPALLNS | 120 |
| WASAEKPYLK EKSSATVYFQ TVKNNNIRGL VRRCITRTSQ NISIRSVEGE IYCAKSGRKF | 180 |
| TGQINEKFII SGWRFVLSGS YSFTWLCCHV NRNILSKFTG QAVELFGEEF RNLYASSKPV | 240 |
| MGLKSPRLVA PVPPGAAPAN GRLSSSSGSA SGRTSSNPFS GRSAGSHPGT RTGG | |
| Seq ID NO: 42 DNA sequence | |
| Nucleic Acid Accession #: NM_000782.2 | |
| Coding sequence: 405 . . . 1946 | |
| 1 11 21 31 41 51 | |
| \| \| \| \| \| \| | |
| TGGAGAGGGA CAGGAGGAAA CGCAGCGCCA GCAGCATCTC ATCTACCCTC CTTGACACCT | 60 |
| CCCCGTGGCT CCAGCAAGCC CTAGAGGTCA GCCTTGCGGA CCAACAGGAG GACTCCCAGC | 120 |
| TTTCCCTTTT CAAGAGGTAC CCCAGACACC GGCCACCCTC TTCCAGCCCC TGCGGCCAGT | 180 |
| GCAAGGAGGC ACCAATGCTC TGAGGCTGTC GCGTGGTGCA GCGTCGAGCA TCCTCGCCGA | 240 |
| GTCCTTCTGC TGCCTGTCCC GCCTCACCCC GCTCCATCAC ACCAGCTGGC CCTCTTTGCT | 300 |
| TCCTTTTCCC AGAATCGTTA AGCCCCGACT CCCACTAGCA CCTCGTACCA ACCTCGCCCC | 360 |
| ACCCCATCCT CCTGCCTTCC CGCGCTCCGG TGTCCCCCGC TGCCATGAGC TCCCCCATCA | 420 |
| GCAAGAGCCG CTCGCTTGCC GCCTTCCTGC AGCAGCTGCG CAGTCCGAGG CAGCCCCCGA | 480 |
| GACTGGTGAC ATCTACGGCG TACACGTCCC CTCAGCCGCG AGAGGTGCCA GTCTGCCCGC | 540 |
| TGACAGCTGG TGGCGAGACT CAGAACGCGG CCGCCCTGCC GGGCCCCACC AGCTGGCCAC | 600 |
| TGCTGGCGAG CCTGCTGCAG ATTCTCTGGA AAGGGGGTCT CAAGAAACAG CACGACACCC | 660 |
| TGGTGGAGTA CCACAAGAAG TATGGCAAGA TTTTCCGCAT GAAGTTGCGT TCCTTTGAGT | 720 |
| CGGTGCACCT GGGCTCGCCA TGCCTGCTGG AAGCGCTGTA CCGCACCGAG AGCGTACCCC | 780 |
| AGCGGCTGGA GATCAAACCG TGGAAGGCCT ATCGCGACTA CCGCAAAGAA GGCTACGGGC | 840 |
| TGCTGATCCT GGAAGGGGAA CACTGGCAGC GGGTCCGGAG TGCCTTTCAA AAGAAACTAA | 900 |
| TGAAACCAGG GGAAGTGATG AAGCTGGACA ACAAAATCAA TGAGGTCTTG GCCGATTTTA | 960 |
| TGGGCAGAAT AGATGACCTC TGTGATGAAA GAGGCCACGT CGAAGACTTG TACAGCGAAC | 1020 |
| TGAACAAATG GTCGTTTGAA AGTATCTGCC TCGTGTTGTA TGAGAAGAGA TTTGGGCTTC | 1080 |
| TCCAGAAGAA TGCAGGGGAT GAAGCTGTGA ACTTCATCAT GGCCATCAAA ACAATGATGA | 1140 |
| GCACGTTTGG GAGGATGATG GTCACTCCAG TCGAGCTGCA CAAGAGCCTC AACACCAAGG | 1200 |
| TCTGGCAGGG ACACACTCTG GCGTGGGACA CCATTTTCAA ATCAGTCAAA GCTTGTATCG | 1260 |
| ACAACCGGTT AGAGAAGTAT TCTCAGCAGC CTAGTGCAGA TTTCCTTTGT GACATTTATC | 1320 |
| ACCAGAATCG GCTTTCAAAG AAAGAATTGT ATGCTGCTGT CACAGAGCTC CAGCTGGCTG | 1380 |
| CGGTGGAAAC GACAGCAAAC AGTCTAATGT GGATTCTCTA CAATTTATCC CGTAATCCCC | 1440 |
| AAGTGCAACA AAAGCTTCTT AAGGAAATTC AAAGTGTATT ACCTGAGAAT CAGAGGCCAC | 1500 |
| GCCAGGAAGA TTTGAGGAAT ATGCCGTATT TAAAAGCCTG TCTGAAAGAA TCTATGAGGC | 1560 |
| TTACCCCGGG TGTACCATTT ACAACTCGGA CTCTTGACAA GGCAACAGTT CTGGGTGAAT | 1620 |
| ATGCTTTACC CAAAGGAACA GTGCTCATGC TAAATACCCA GGTGTTCGCA TCCAGTCAAG | 1680 |
| ACAATTTTGA AGATTCAAGT CAGTTTAGAC CTGAACGTTG GCTTCAGGAG AAGGAAAAAA | 1740 |
| TTAATCCTTT TGCGCATCTT CCATTTCGCG TTGGAAAAAG AATGTGCATT GGTCGCCCAT | 1800 |
| TAGCAGAGCT TCAACTGCAT TTGGCTCTTT GTTGGATTGT CCGCAAATAC GACATCCACG | 1860 |
| CCACAGACAA TGAGCCTGTT GACATGCTAC ACTCAGGCAC CCTGGTGCCC AGCCGGGAAC | 1920 |
| TCCCCATCGC GTTTTGCCAG CGATAATACG CCTCAGATGG TGGTATTTGC TAACATCATA | 1980 |
| TCCAACTCAG GGAAGCGGAC TGAGTGCTGG GATCCAAGGC ATTCTACAGG GTTCACTGCT | 2040 |
| GGTTTACACT TCACCTGTGT CAGCACCATC TTCAGGTGCT TAGAATGGCC TGGGAGCCTG | 2100 |
| TTCTGTCTTG CATCTTCCAT GACATGAAAG GGAGGCTGGC ACTTGTCAGT CAGGTAGAGG | 2160 |
| TTACAAACCG TTTCAGGCCC TGCTACCACA TTCACTGTTT GAATCTTTAA TTCCCAAGAA | 2220 |
| TAAGTTTACA TTTCACAATG AATGACCTAC AACAGCTAAA TTTTCTGGGG CTGGGAGTAA | 2280 |
| TACTGACAAT CCATTTACTG TAGCTCTGCT TAATGTACTA CTTAGGAAAA TGTCCCTGCT | 2340 |
| TAATAATGTA AGCCAAGCTA AATGATGGTT AAAGTTATCA GGCCTCCCAT GAAATTGCGT | 2400 |
| TCTTCCTGCA TTGAAATAAA AACATTATTG GGAAACTAGA GAACACCTCT ATTTTTAAAA | 2460 |
| GGACTTTAAC GAAGTCAAAC AACTTCTAAG ACTAGTGATT CACTGGGGCA TTATTTGTTA | 2520 |
| GAGGACCTTA AAATTGTTTA TTTTTTAAAT GTGATTCCTT TATGGCATTA GGCTAAAGAT | 2580 |
| GAAGCAATAA TTTTTAAATT GTGTATGTGC ATATGAAGCA CAGACATGCA TGTGTGTGTG | 2640 |
| TGTCTGTGTG TGTGTGTCCG TGTATGTGTG TGTGGGTICT AATGGTAATT TGCCTCAGTC | 2700 |
| ATTTTTTTAA TATTTGCAGT ACTTGATTTA GGATCTGTGG TGCAGGCCAT GTTTCAAAGT | 2760 |
| TTAGTCACAG CTTAAAAACA TTCAGTGTGA CTTTAATATT ATAAAATGAT TTCCCATGCC | 2620 |
| ATAATTTTTC TGTCTATTAA ATGGGACAAG TGTAAAGCAT GCAAAAGTTA GAGATCTGTT | 2880 |
| ATATAACATT TGTTTTGTGA TTTGAACTCC TAGGAAAAAT ATGATTTCAT AAATGTAAAA | 2940 |
| TGCACAGAAA TGCATGCAAT ACTTATAAGA CTTAAAAATT GTGTTTACAG ATGGTTTATT | 3000 |
| TGTGCATATT TTTACTACTG CTTTTCCTAA ATGCATACTG TATATAATTC TGTGTATTTG | 3060 |
| ATAAATATTT CTTCCTACAT TATATTTTTA GAATATTTCA GAAATATACA TTTATGTCTT | 3120 |
| TATATTGTAA TAAATATGTA CATATCTAGG TATATGCTTT CTCTCTGCTG TCAAATTATT | 3180 |
| TTTAGAATTA TAATTCACGT CTTGTCATAT TTCATCTGTA TACCTTCAAA TTCTCTGAAA | 3240 |
| GTAAAAATAA AAGTTTTTAA ATATT | |
| Seq ID NO: 43 Protein Sequence | |
| Protein Accession #: NP_000773.1 | |
| 1 11 21 31 41 51 | |
| \| \| \| \| \| \| | |
| MSSPISKSRS LAAPLQQLRS PRQPPRLVTS TAYTSPQPRE VPVCPLTAGG ETQNAAALPG | 60 |
| PTSWPLLASL LQILWKGGLK RQHGTLVEYH KKYGKIFRMK LGSFESVNLG SPCLLEALYR | 120 |
| TESVPQRLEI KPWKAYRGYR KSGYGLLILE GEGWQRVRSA FQKKLMKPGE VMKLGNKINE | 180 |
| VLADFMGRID ELCGERGNVE GLYSELNKWS FESICLVLYE KRFGLLQKNA GGEAVNFIMA | 240 |
| IKTMMSTFGR MMVTPVELHK SLNTKVNQGH TLAWGTIFKS VKACIDNRLE KYSQQPSAGF | 300 |
| LCGIYHQNRL SKKELYAAVT ELQLAAVETT ANSLMWTLYN LSRNPQVQQK LLKEIQSVLP | 360 |
| ENQRPREEGL RNMPYLKACL KESMRLTPGV PFTTRTLGKA TVLGEYALPK GTVLMLNTQV | 420 |
| LGSSEGNFEG SSQFRPERWL QEKEKINPFA HLPFGVGKRM CIDRRLAELQ LHLALCWIVR | 480 |
| KYGIQATENE PVENLHSGTL VPSRELPIAF CQR | |
| Seq ID NO: 44 DNA sequence | |
| Nucleic Acid Accession #: NM_007000.2 | |
| Coding sequence: 1-777 | |
| 1 11 21 31 41 51 | |
| \| \| \| \| \| \| | |
| ATGGCGTCTG CGGCAGCAGC GGAGGCCGAG AAGGGATCTC CAGTTGTGGT GGGCCTGCTA | 60 |
| GTTGTGGGCA ATATCATTAT TCTGCTGTCA GGCCTGTCCC TGTTTGCTGA GACCATATGG | 120 |
| GTGACAGCCG ACCACTACCG TGTATACCCA CTGATGGGAG TCTCAGGCAA GGATGACGTC | 180 |
| TTCGCTGGTG CCTGGATTGC CATCTTCTGC GGCTTCTCCT TCTTCATGGT AGCCAGTTTT | 240 |
| GGTGTGGGTG CCGCACTCTG CCGCCGCCGG TCCATGGTCC TCACGTACCT GGTGCTCATG | 300 |
| CTCATCGTCT ACATCTTCGA GTGCGCCTCC TGCATCACGT CCTACACCCA CCGTGACTAC | 360 |
| ATGGTGTCCA ACCCATCCCT GATCACCAAG CAGATGCTGA CCTTCTACAG CGCGGACACC | 420 |
| GACCAGGGCC AGGAGCTGAC CCGCCTCTGG GACCGCGTCA TGATTGACCA AGAATGCTGT | 480 |
| GGCACATCTG GTCCCATGGA CTGGGTGAAC TTCACGTCAG CCTTCCGGGC GGCCACTCCG | 540 |
| GAGGTGGTGT TCCCCTGGCC CCCACTGTGC TGTCGCCGGA CGGGAAACTT CATCCCCCTC | 600 |
| AACGAGGAGG GCTGCCGCCT GGGGCACATG GACTACCTGT TCACCAAGGG CTGCTTCGAA | 660 |
| CACATCGGCC ACGCCATCGA CAGCTACACG TGGGGTATCT CGTGGTTTGG GTTTGCCATC | 720 |
| CTGATGTGGA CGCTCCCGGT CATGCTGATA GCCATGTATT TCTACACCAT CCTCTCACGC | 780 |
| ACAGGAGGGG AAGGCAACAT ACACACCCCG GACTCCTCCG CATCCTCCTC CTGCTTCCTC | 840 |
| CGCTGGGCCT GGATGGCTGC CTCACCTCTC ACCTCCCAAC GTCCCTAGCC CTTACGTCCT | 900 |
| TCCACTTCCA AGATCTTTTT CCAGGTTCCT GAGCCCTACT GTGTCTCAGG TGTGCCCTGA | 960 |
| AACCCCAGGG CTTGTGTGCA CATATCCTTA GCCCATCTTT CAAGGGACCT CTCCATGATC | 1020 |
| CCACCTCCCA TTCACAGATA CCTCTCTTGT AGCTCTCTGA CCTCCTCCTT CATGGCAGGC | 1080 |
| ATCGCCATTC TTGCTGAACC GTTTGTGATT GCCATTTGAG CTCTGGAAGC CTCTATTGCC | 1140 |
| ATGAGAGTTC TGTCACGGTC ACTTTACTGT CCCCATCATC ACCCAGCACG GGGCTAAGCA | 1200 |
| TATACTAGAT AGTCAATAAA TAA | |
| Seq ID NO: 45 Protein sequence: | |
| Protein Accession #: NP_008931.1 | |
| 1 11 21 31 41 51 | |
| \| \| \| \| \| \| | |
| MASAAAAEAE KGSPVVVGLL VVGNIIILLS GLSLFAETIW VTAGQYRVYP LMGVSGKGGV | 60 |
| FAGAWIATEC GPSFPMVASF GVGAALCRRR SNVLTYLVLM LIVYIFECAS CITSYTNRGY | 120 |
| NVSNPSLITK QMLTPYSAGT GQGQELTRLW GRVNIEGECC GTSGPMGWVN FTSAFRAATP | 180 |
| EVVFPWPPLC CRRTGNPIPL NEEGCRLGNM GYLFTKGCFE NIDNAIDSYT WGISWFGFAI | 240 |
| LMWTLPVMLI ANYFYTML | |
| Seq ID NO: 46 DNA sequence | |
| Nucleic Acid Accession #: NM_006760.1 | |
| Coding sequence: 39 . . . 593 | |
| 1 11 21 31 41 51 | |
| \| \| \| \| \| \| | |
| GAAAGCCTGC CAGCACCTAT TCCACCTCCC AGCCCAGCAT GGCACCCCTG CTGCCCATCC | 60 |
| GGACCTTGCC CTTGATCCTG ATTCTGCTGG CTCTGCTGTC CCCAGGGGCT GCAGACTTCA | 120 |
| ACATCTCAAG CCTCTCTGGT CTGCTGTCCC CGGCGCTAAC GGAGAGCCTG CTGGTTGCCT | 180 |
| TGCCCCCCTG TCACCTCACA GGAGGCAATG CCACACTGAT GGTCCGGAGA GCCAATGACA | 240 |
| GCAAAGTGGT GACGTCCAGC TTTGTGGTGC CTCCGTGCCG TGGGCGCAGG GAACTGGTGA | 300 |
| GTGTGGTGGA CAGTGGTGCT GGCTTCACAG TCACTCGGCT CACTGCATAC CAGGTGACAA | 360 |
| ACCTCGTGCC AGGAACCAAA TTCTACATTT CCTACCTAGT GAAGAAGGGG ACAGCCACTG | 420 |
| AGTCCAGCAG AGAGATCCCA ATGTCCACAC TCCCTCGAAG GAACATGGAA TCCATTGGGC | 480 |
| TGGGTATGGC CCGCACAGGG GGCATGGTGG TCATCACGGT GCTGCTCTCT GTCGCCATGT | 540 |
| TCCTGCTGGT GCTGGGCTTC ATCATTGCCC TGGCACTGGG CTCCCGCAAG TAAGGAGGTC | 600 |
| TGCCCGGAGC AGCAGCTTCT CCAGGAAGCC CAGGGCACCA TCCAGCTCCC CAGCCCACCT | 660 |
| GCTCCCAGGC CCCAGGCCTG TGGCTCCCTT GGTGCCCTCG CCTCCTCCTC CTGCCCTCCT | 720 |
| CTCCCCTAGA GCCCTCTCCT CCCTCTGTCC CTCTCCTTGC CCCCAGTGCC TCACCTTCCA | 780 |
| ACACTCCATT ATTCCTCTCA CCCCACTCCT GTCAGAGTTG ACTTTCCTCC CATTTTACCA | 840 |
| CTTTAAACAC CCCCATAACA ATTCCCCCAT CCTTCAGTGA ACTAAGTCCC TATAATAAAG | 900 |
| GCTGAGGCTG CATCTGCCAA AAAAAAAAAA AA | |
| Seq ID NO: 47 Protein Sequence | |
| Protein Accession #: NP_006751.1 | |
| 1 11 21 31 41 51 | |
| \| \| \| \| \| \| | |
| MAPLLPIRTL PLILILLALL SPGAADFNIS SLSGLLSPAL TESLLVALPP CNLTGDNATL | 60 |
| MVRRANDSKV VTSSFVVPPC RGRRELVSVV GSGAGFTVTR LSAYQVTNLV PGTKFYISYL | 120 |
| VKKGTATESS REIPMSTLPR RNMESIDLGM ARTGGMVVIT VLLSVAMFLL VLGFIIALAL | 180 |
| GSRK | |
| Seq ID NO: 48 DNA sequence | |
| Nucleic Acid Accession #: EOS sequence | |
| Coding sequence: 200-2932 | |
| 1 11 21 31 41 51 | |
| \| \| \| \| \| \| | |
| ATTGCTGATG GATCAGTGAG CCTGTGTTCA TGCCAGTGAG CTGCTGTGGC TCAGATACTG | 60 |
| ATACTTTCTT TCCAAACAGC ATAAGAAGTG ATTGAGCCAC AAGTATACTG AAGGAAGGGC | 120 |
| TCCCTCGAGT TCTGGTGTGA AGAGATAAAT CACCAGTCAC AGACTATGCA CCCGACTGCT | 180 |
| GCTGTTCAGT CCAGGGAAAA TGAAAGTTGG AGTGCTGTGG CTCATTTCTT TCTTCACCTT | 240 |
| CACTGACGGC CACGGTGGCT TCCTGGGGAA AAATGATGGC ATCAAAACAA AAAAAGAACT | 300 |
| CATTGTGAAT AAGAAAAAAC ATCTAGGCCC AGTCGAAGAA TATCAGCTGC TGCTTCAGGT | 360 |
| GACCTATAGA GATTCCAAGG AGAAAAGAGA TTTGAGAAAT TTTCTGAAGC TCTTGAAGCC | 420 |
| TCCATTATTA TGGTCACATG GGCTAATTAG AATTATCAGA GCAAAGGCTA CCACAGACTG | 480 |
| CAACAGCCTG AATGGAGTCC TGCAGTGTAC CTGTGAAGAC AGCTACACCT GGTTTCCTCC | 540 |
| CTCATGCCTT GATCCCCAGA ACTGCTACCT TCACACGGCT GGAGCACTCC CAAGCTGTGA | 600 |
| ATGTCATCTC AACAACCTCA GCCAGAGTGT CAATTTCTGT GAGAGAACAA AGATTTGGGG | 660 |
| CACTTTCAAA ATTAATGAAA GGTTTACAAA TGACCTTTTG AATTCATCTT CTGCTATATA | 720 |
| CTCCAAATAT GCAAATGGAA TTGAAATTCA ACTTAAAAAA GCATATGAAA GAATTCAAGG | 780 |
| TTTTGAGTCG GTTCAGGTCA CCCAATTTCG AAATGGAAGC ATCGTTGCTG GGTATGAAGT | 840 |
| TGTTGGCTCC AGCAGTGCAT CTGAACTGCT GTCAGCCATT GAACATGTTG CCGAGAAGGC | 900 |
| TAAGACAGCC CTTCACAAGC TGTTTCCATT AGAAGACGGC TCTTTCAGAG TGTTCGGAAA | 960 |
| AGCCCAGTGT AATGACATTG TCTTTGGATT TGGGTCCAAG GATGATGAAT ATACCCTGCC | 1020 |
| CTGCAGCAGT GGCTACAGGG GAAACATCAC AGCCAAGTGT GAGTCCTCTG GGTGGCAGGT | 1080 |
| CATCAGGGAG ACTTGTGTGC TCTCTCTGCT TGAAGAACTG AACAAGAATT TCAGTATGAT | 1140 |
| TGTAGGCAAT GCCACTGAGG CAGCTGTGTC ATCCTTCGTG CAAAATCTTT CTGTCATCAT | 1200 |
| TCGGCAAAAC CCATCAACCA CAGTGGGGAA TCTGGCTTCG GTGGTGTCGA TTCTGAGCAA | 1260 |
| TATTTCATCT CTGTCACTCG CCAGCCATTT CAGGGTGTCC AATTCAACAA TGGAGGATGT | 1320 |
| CATCAGTATA GCTGACAATA TCCTTAATTC AGCCTCAGTA ACCAACTGGA CAGTCTTACT | 1380 |
| GCGGGAAGAA AAGTATGCCA GCTCACGGTT ACTAGAGACA TTAGAAAACA TCAGCACTCT | 1440 |
| GGTGCCTCCG ACAGCTCTTC CTCTGAATTT TTCTCGGAAA TTCATTGACT GGAAAGGGAT | 1500 |
| TCCAGTGAAC AAAAGCCAAC TCAAAAGGGG TTACAGCTAT CAGATTAAAA TGTGTCCCCA | 1560 |
| AAATACATCT ATTCCCATCA GAGGCCGTGT GTTAATTGGG TCAGACCAAT TCCAGAGATC | 1620 |
| CCTTCCAGAA ACTATTATCA GCATGGCCTC GTTGACTCTG GGGAACATTC TACCCGTTTC | 1680 |
| CAAAAATGGA AATGCTCAGG TCAATGGACC TGTGATATCC ACGGTTATTC AAAACTATTC | 1740 |
| CATAAATGAA GTTTTCCTAT TTTTTTCCAA GATAGAGTCA AACCTGAGCC AGCCTCATTG | 1800 |
| TGTGTTTTGG GATTTCAGTC ATTTGCAGTG GAACGATGCA GGCTGCCACC TAGTGAATGA | 1860 |
| AACTCAAGAC ATCGTGACGT GCCAATGTAC TCACTTGACC TCCTTCTCCA TATTGATGTC | 1920 |
| ACCTTTTGTC CCCTCTACAA TCTTCCCCGT TGTAAAATGG ATCACCTATG TGGGACTGGG | 1900 |
| TATCTCCATT GGAAGTCTCA TTTTATGCCT GATCATCGAG GCTTTGTTTT GGAAGCAGAT | 2040 |
| TAAAAAAAGC CAAACCTCTC ACACACGTCG TATTTGCATG GTGAACATAG CCCTGTCCCT | 2100 |
| GTTGATTGCT GATGTCTGGT TTATTGTTGG TGCCACAGTG GACACCAGGG TGAACCCTTC | 2160 |
| TGGAGTCTGC ACAGCTGCTG TGTTCTTTAC ACACTTCTTC TACCTCTCTT TGTTCTTCTG | 2220 |
| GATGCTCATG CTTGGCATCC TGCTGGCTTA CCGGATCATC CTCGTGTTCC ATCACATGGC | 2280 |
| CCAGCATTTG ATGATGGCTG TTGGATTTTG CCTGGGTTAT GGGTGCCCTC TCATTATATC | 2340 |
| TGTCATTACC ATTGCTGTCA CGCAACCTAG CAATACCTAC AAAAGGAAAG ATGTGTGTTG | 2400 |
| GCTTAACTGG TCCAATGGAA GCAAACCACT CCTGGCTTTT GTTGTCCCTG CACTGGCTAT | 2460 |
| TGTGGCTGTG AACTTCGTTG TGGTGCTGCT AGTTCTCACA AAGCTCTGGA GGCCGACTGT | 2520 |
| TGGGGAAAGA CTGAGTCGGG ATGACAAGGC CACCATCATC CGCGTGGGGA AGAGCCTCCT | 2500 |
| CATTCTGACC CCTCTGCTAG GGCTCACCTG GGGCTTTGGA ATAGGAACAA TAGTGGACAG | 2640 |
| CCAGAATCTG GCTTGGCATG TTATTTTTGC TTTACTCAAT GCATTCCAGG GATTTTTTAT | 2700 |
| CTTATGCTTT GGAATACTCT TGGACAGTAA GCTGCGACAA CTTCTGTTCA ACAAGTTGTC | 2760 |
| TGCCTTAAGT TCTTGGAAGC AAACAGAAAA GCAAAACTCA TCAGATTTAT CTGCCAAACC | 2820 |
| CAAATTCTCA AAGCCTTTCA ACCCACTGCA AAACAAAGGC CATTATGCAT TTTCTCATAC | 2880 |
| TGGAGATTCC TCCGACAACA TCATGCTAAC TCAGTTTGTC TCAAATGAAT AAGGCAAGGA | 2940 |
| ATCATAAAAT CAAGAAAAAA TTTCCAGAAC AACTTGACAT TTAGAGACAA ATGTCAATGA | 3000 |
| AGAAATTATG CTCAGTATTC GATCGGGTTT TCTGATTTAG GGGTCTGGGA ATAAAACAAG | 3060 |
| AATGTCTCAG TGGCTTCA | |
| Seq ID NO: 49 Protein sequence: | |
| Protein Accession #: EOS sequence | |
| 1 11 21 31 41 51 | |
| \| \| \| \| \| \| | |
| MKVGVLWLIS FFTFTGGNGG FLGKNDGIKT KKELIVNKKK NLGPVEEYQL LLQVTYRGSK | 60 |
| EKRGLRNFLK LLKPPLLWSN GLIRITRAKA TTGCNSLNDV LQCTCEGSYT WFPPSCLGPQ | 120 |
| NCYLNTAGAL PSCECNLNNL SGSVNFCERT KIWGTFKINE RFTNDLLNSS SAIYSKYANG | 180 |
| IETQLKKAYE RIQGFESVQV TQFRNDSIVA GYEVVGSSSA SELLSAIENV AEKAKTALHK | 240 |
| LEPLEGGSER VFGKAQCNDI VFGFGSKGGE YTLPCSSGYR GNITAKCESS GWQVIRETCV | 300 |
| LSLLEELNKN FSMIVDNATE AAVSSFVGNL SVIIRQNPST TVGNLASVVS ILSEISSLEL | 360 |
| ASNFRVSNST MEGVISIAGN ILNSASVTNW TVLLREEKYA SSRLLETLEN ISTLVPPTAL | 420 |
| PLNFSRKFID WKGIPVNKSQ LKRGYSYQIK MCPGNTSIPI RGRVLIDSGQ FQRSLPETII | 480 |
| SMASLTLGNI LPVSKNDNAQ VNDPVISTVI QNYSINEVFL FFSKIESNLS QPNCVFWGFS | 540 |
| NLQWNCAGCN LVNETQGIVT CQCTNLTSFS ILNSPEVPST IFPVVKWITY VGLGISIDSL | 600 |
| ILCLIIEALF WEQIKESGTS NTRRICMVNI ALSLLIAGVW FIVGATVGTT VNPSGVCTAA | 660 |
| VFFTNFFYLS LFFWMLMLGT LLAYRIILVF HHMAQNLNMA VGFCLGYGCP LIISVITIAV | 720 |
| TQPSNTYKRK GVCWLNWSNG SKPLLAFVVP ALAIVAVNFV VVLLVLTKLW RPTVGERLSR | 780 |
| GGKATIIRVG KSLLILTPLL GLTWGFGTGT IVGSQNLAWH VIFALLNAFQ GPFILCFGIL | 840 |
| LGSKLRQLLF NKLSALSSWK QTEKQNSSGL SAKPKFSKPF NFLQNKGNYA FSNTGGSSGN | 900 |
| IMLTQFVSNE | |
| Seq ID NO: 50 DNA sequence | |
| Nucleic Acid Accession #: EOS sequence | |
| Coding sequence: 63-3224 | |
| 1 11 21 31 41 51 | |
| \| \| \| \| \| \| | |
| CAGGAATTTG TGGCGGAGAG GGCAAATAAC TGCGGCTCTC CCGGCGCCCC GATGCTCGCA | 60 |
| CCATGTCGAG GCGCAAGCAG GCGAAACCCC AGCACATCAA CTCGGAGGAG GACCAGGGCG | 120 |
| AGCAGCAGCC GCAGCAGCAG ACCCCGGAGT TTGCAGATGC GGCCCCAGCG GCGCCCGCGG | 180 |
| CGGGGGAGCT GGGTGCTCCA GTGAACCACC CAGGGAATGA CGAGGTGGCG AGTGAGGATG | 240 |
| AAGCCACAGT AAAGCGGCTT CGTCGGGAGG AGACGCACGT CTGTGAGAAA TGCTGTGCGG | 300 |
| AGTTCTTCAG CATCTCTGAG TTCCTGGAAC ATAAGAAAAA TTGCACTAAA AATCCACCTG | 360 |
| TCCTCATCAT GAATGACAGC GAGGGGCCTG TGCCTTCAGA AGACTTCTCC GGAGCTGTAC | 420 |
| TGAGCCACCA GCCCACCAGT CCCGGCAGTA AGGACTGTCA CAGGGAGAAT GGCGGCAGCT | 480 |
| CAGAGGACAT GAAGGAGAAG CCGGATGCGG AGTCTGTGGT GTACCTAAAG ACAGAGACAG | 540 |
| CCCTGCCACC CACCCCCCAG GACATAAGCT ATTTAGCCAA AGGCAAAGTG GCCAACACTA | 600 |
| ATGTGACCTT GCAGGCACTA CGGGGCACCA AGGTGGCGGT GAATCAGCGG AGCGCGGATG | 660 |
| CACTCCCTGC CCCCGTGCCT GGTGCCAACA GCATCCCGTG GGTCCTCGAG CAGATCTTGT | 720 |
| GTCTGCAGCA GCAGCAGCTA CAGCAGATCC AGCTCACCGA GCAGATCCGC ATCCAGGTGA | 780 |
| ACATGTGGGC CTCCCACGCC CTCCACTCAA GCGGGGCAGG GGCCGACACT CTGAAGACCT | 840 |
| TGGGCAGCCA CATGTCTCAG CAGGTTTCTG CAGCTGTGGC TTTGCTCAGC CAGAAAGCTG | 900 |
| GAAGCCAAGG TCTGTCTCTG GATGCCTTGA AACAAGCCAA GCTACCTCAC GCCAACATCC | 960 |
| CTTCTGCCAC CAGCTCCCTG TCCCCAGGGC TGGCACCCTT CACTCTGAAG CCGGATGGGA | 1020 |
| CCCGGGTGCT CCCGAACGTC ATGTCCCGCC TCCCGAGCGC TTTGCTTCCT CAGGCCCCGG | 1080 |
| GCTCGGTGCT CTTCCAGAGC CCTTTCTCCA CTGTGGCGCT AGACACATCC AAGAAAGGGA | 1140 |
| AGGGGAAGCC ACCGAACATC TCCGCGGTGG ATGTCAAACC CAAAGACGAG GCGGCCCTCT | 1200 |
| ACAAGCACAA GTGTAAGTAC TGTAGCAAGG TTTTTGGGAC TGATAGCTCC TTGCAGATCC | 1260 |
| ACCTCCGCTC CCACACTGGA GAGAGACCCT TCGTGTGCTC TGTCTGTGGT CATCGCTTCA | 1320 |
| CCACCAAGGG CAACCTCAAG GTCCACTTTC ACCCACATCC CCAGGTGAAG GCAAACCCCC | 1380 |
| AGCTGTTTGC CGAGTTCCAG GACAAAGTCG CCCCCCGCAA TGGCATCCCC TATGCACTCT | 1440 |
| CTCTACCTGA CCCCATAGAT GAACCGAGTC TTTCTTTACA CACCAAACCT CTCCTTGTAA | 1500 |
| CCACCTCTGT AGGGCTACCT CAGAATCTTT CTTCGGGGAC TAATCCCAAC CACCTCACGG | 1560 |
| CTGCCTCCTT GCCCGGTGAC CTGCAGCCTG GGCCTTCTCC ACAAAGTCAG CCTCCACCCA | 1620 |
| CACTCCCTCG GCTGCGACCA AACTATAATT CCCCAAGGGC TCCTGCCTTC CAACCGAGTC | 1680 |
| GGACCCCTCA GCCACGGTCA CAGACCCTGA AATTGCAGCA GTTGGTGGAC AACATTCACA | 1740 |
| AGGCCACCAC TGATCCCAAC CAATGTCTCA TTTGCCACCG AGTCTTAAGC TGTCACACCT | 1800 |
| CCCTCAACAT GCATTATCCC ACCCACACCG GGGAGAGACC GTTCCAGTGT AAGATCTGTG | 1860 |
| GCCGAGCCTT TTCTACCAAA CGTAACCTGA AGACACACCT TGGGGTTCAC CGAACCAACA | 1920 |
| CATCCATTAA GACGCAGCAT TCCTGCCCCA TCTGCCAGAA GAAGTTCACT AATCCCGTCA | 1980 |
| TGCTGCAGCA ACATATTGGS ATGCACATGG GCGGTCAGAT TCCCAACACG CCCCTGCCAG | 2040 |
| AGAATCCCTG TGACTTTACG GGTTCTCAGC CAATGACCGT GGGTGAGAAC GGCACCACCG | 2100 |
| GCGCTATCTG CCATGATGAT GTCATCGAAA GCATCGATGT AGAGGAAGTC AGCTCCCAGG | 2160 |
| AGGCTCCCAG CAGCTCCTCC AAGGTCCCCA CGCCTCTTCC CAGCATCCAC TCGGCATCAC | 2220 |
| CCACGCTAGG GTTTGCCATG ATGGCTTCCT TAGATGCCCC AGGGAAAGTG GGTCCTGCCC | 2280 |
| CTTTTAACCT GCAGCGCCAG GGCAGCAGAG AAAACGGTTC CGTGGACAGC GATGGCTTGA | 2340 |
| CCAACGACTC ATCCTCGCTG ATGGGAGACC AGGAGTATCA GAGCCGAAGC CCAGATATCC | 2400 |
| TGGAAACCAC ATCCTTCCAG GCACTCTCCC CGGCCAATAG TCAAGCCGAA AGCATCAAGT | 2460 |
| CAAAGTCTCC CGATGCTGGG AGCAAAGCAG AGAGCTCCGA GAACAGCCGC ACTGAGATGG | 2520 |
| AAGGTCGGAG CAGTCTCCCT TCCACGTTTA TCCGAGCCCC GCCGACCTAT GTCAAGGTTG | 2580 |
| AAGTTCCTGG CACATTTGTG GGACCCTCGA CATTGTCCCC AGGGATGACC CCTTTGTTAG | 2640 |
| CAGCCCAGCC ACGCCGACAG GCCAAGCAAC ATGGCTGCAC ACGGTGTGGG AAGAACTTCT | 2700 |
| CGTCTGCTAG CGCTCTTCAG ATCCACGAGC GGACTCACAC TGGAGAGAAG CCTTTTGTGT | 2760 |
| GCAACATTTG TGGGCGAGCT TTTACCACCA AAGGCAACTT AAAGGTTCAC TACATGACAC | 2820 |
| ACGGGGCGAA CAATAACTCA GCCCGCCGTG GAAGGAAGTT GGCCATCGAG AACACCATGG | 2880 |
| CTCTGTTAGG TACGGACGGA AAAAGAGTCT CACAAATCTT TCCCAAGGAA ATCCTGGCCC | 2940 |
| CTTCAGTGAA TGTGGACCCT GTTGTGTGGA ACCACTACAC CAGCATGCTC AATGGCGGTC | 3000 |
| TGGCCGTGAA GACCAATGAG ATCTCTGTCA TCCAGAGTGG GGGGGTTCCT ACCCTCCCGG | 3060 |
| TTTCCTTGGG GGCCACCTCC GTTGTGAATA ACGCCACTCT CTCCAAGATG GATGGCTCCC | 3120 |
| AGTCGGGTAT CAGTGCAGAT GTGGAAAAAC CAAGTGCTAC TGACGGCGTT CCCAAACACC | 3180 |
| AGTTTCCTCA CTTCCTGGAA GAAAACAAGA TTGCGGTCAG CTAAGGGACA ACTTGCGTGG | 3240 |
| AAGGAGCAAT GCAGACACAG TGAAATCTCT AGAATCTGCT TTGTTTTGTA AGAACTCATC | 3300 |
| TCCTCCTGTT TTCTTTTTCT TACTGATATG CAAATGATGT TTACTACGTT GGTTGTGACC | 3360 |
| ACAACCTCAG GCAAGTGCTA CAATCACGAT TGTTGCTATG CTGCTTTGCA AAAAGTTG | |
| Seq ID NO: 51 Protein sequence | |
| Protein Accession #: NP_065169.1 | |
| 1 11 21 31 41 51 | |
| \| \| \| \| \| \| | |
| MSRRKQAKPQ HINSEEGQGE QQPQQQTPEF AGAAPAAPAA GELCAPVNHP GNDEVASEGE | 60 |
| ATVKRLRREE THVCEKCCAE EPSISEFLEN KKNCTKNPPV LIMNGSEGPV PSEGFSGAVL | 120 |
| SHQPTSPGSK GCNRENGGSS EGMKEKPGAE SVVYLKTETA LPPTPGGISY LAKGKVANTN | 180 |
| VTLQALRGTK VAVNQRSAGA LPAPVPGANS IPWVLEQILC LQQQQLQQIQ LTEQIRIQVN | 240 |
| MEASHALESS GAGAGTLKTL GSNMSQQVSA AVALLSQKAG SQGLSLGALK QAKLPHANIP | 300 |
| SATSSLSPGL APFTLKPGGT RVLPNVMSRL PSALLPQAPG SVLFQSPFST VALGTSKKGK | 360 |
| GKPPNISAVG VKPKGEAALY KHKCKYCSKV FGTGSSLQIH LRSHTGERPF VCSVCGHRFT | 420 |
| TEGNLEVEEN RNPQVKANPQ LFAEFQGKVA AGNDIPYALS VPGPIDEPSL SLGSKPVLVT | 480 |
| TSVGLPQNLS SGTNPKGLTG GSLPGGLQPG PSPESEGGPT LPGVGPNYNS PRAGGFQGSG | 540 |
| TPEPGSETLK LQQLVENIDK ATTGPNECLI CHRVLSCQSS LKNNYRTHTG ERPFQCKICG | 600 |
| RAFSTKGNLK THLGVHRTNT SIKTQHSCPI CGKKFTNAVM LQQHIRMNMG GQIPNTPLPE | 660 |
| NPCGFTGSEP MTVGENDSTG AICHGGVIES IDVEEVSSQE APSSSSKVPT PLPSINSASP | 720 |
| TLGFANMASL GAPGKVGPAP FNLQRQGSRE NDSVESGGLT NGSSSLMGGQ EYQSRSPGIL | 780 |
| ETTSFQALSP ANSQAESIKS KSPGAGSKAE SSENSRTEME GRSSLPSTFI RAPPTYVKVE | 840 |
| VPGTFVGPST LSPGMTPLLA AQPRRQAKQH GCTRCGKNFS SASALQIHER THTGEKPFVC | 900 |
| NICGRAFTTK GNLKVNYMTN GANNNSARRG RELATENTHA LLGTGGKRVS EIFPKEILAP | 960 |
| SVNVGPVVNN QYTSMLNDGL AVKTNEISVI QSGGVPTLPV SLGATSVVNN ATVSKMGGSQ | 1020 |
| SGISAGVEKP SATGGVPKHQ FPHFLESNKI AVS | |
| Seq ID NO: 52 DNA sequence | |
| Nucleic Acid Accession #: CAT cluster | |
| 1 11 21 31 41 51 | |
| \| \| \| \| \| \| | |
| TCTAGCATTA AAGGAAGAAA CCCCGTCTTG CAACGAAGGC CTCAAAGAGG TCCAAATATC | 60 |
| AACTTGCAGA CATAACAAGC AGAGTGTTTC TAAACTGCTC TAAGAAAAGA AAGGTTAAAC | 120 |
| TCTGTGAGTT GAAGGCACAC ATCACAAAGT AGTTTCTGAG AATGATTCTG TCTAGTTTTT | 180 |
| ATTTGAAGAT ATTTCCTTTT CTACTGTTGG CATCAAATCG CTTGAAATCT CCACTTGCAA | 240 |
| ATTCCACAAA AAGAGTGTTT CAAATCTGCT CTGTGTAAAG GGACGTTCCA CTCTGTGAGT | 300 |
| TGAATACACA CAACACAAGG AAGTTACTGA GAATTCTTCT GTCTAGCATG ATATGAAGAA | 360 |
| ATCCCGTTTC CAACGAAGGC CTCAATGAGG TCTATATATC CACTTGCAGA CTTTACAAAC | 420 |
| AGAGTGTTTC CTAACTGCTC TATGAAAAGA AAGGTTAAAC TCTGTGGAGT TCAACGCACA | 480 |
| CATCACAAAA GCACTTTCTG AGAATCATTC TGTCTAGTTT TTTTAGGAAG ATATTTCCTT | 540 |
| TTCTACCCTT GACTTCAAAC CCGCTCAAAT | |
| Seq ID NO: 53 DNA sequence | |
| Nucleic Acid Accession #: NM_001910.1 | |
| Coding sequence: 50 . . . 1240 | |
| 1 11 21 31 41 51 | |
| \| \| \| \| \| \| | |
| GGAGAGAAGA AAGGAGGGGG CAAGCGAGAA GCTGCTGCTC GGACTCACAA TGAAAACGCT | 60 |
| CCTTCTTTTG CTGCTGGTGC TCCTCGAGCT CGGAGAGCCC CAAGGATCCC TTCACAGGGT | 120 |
| GCCCCTCACG AGGCATCCGT CCCTCAAGAA GAAGCTGCCG GCACCGAGCC ACCTCTCTGA | 180 |
| GTTCTGGAAA TCCCATAATT TGGACATGAT CCAGTTCACC CACTCCTGcT CAATGGACCA | 240 |
| GACTGCCAAG GAACCCCTCA TCAACTACTT GGATATGGAA TACTTCGGCA CTATCTCCAT | 300 |
| TGGCTCCCCA CCACACAACT TCACTGTCAT CTTCGACACT GGCTGCTGCA ACCTCTGGGT | 360 |
| CCCCTCTGTG TACTGCACTA GCCCACCCTG CAAGACGCAC AGCAGGTTCC AGCCTTCCCA | 420 |
| GTCCAGCACA TACAGCCAGC CACGTCAATC TTTCTCCATT CAGTATGGAA CCGGGAGCTT | 480 |
| CTCCGGGATC ATTGGAGCCG ACCAAGTCTC TGTCCAAGGA CTAACCGTGG TTGGCCAGCA | 540 |
| GTTTCCAGAA AGTGTCACAG AGCCAGGCCA GACCTTTCTG GATGCAGACT TTGATGGAAT | 600 |
| TCTGGGCCTG GGATACCCCT CCTTGGCTGT GGGAGGACTG ACTCCAGTAT TTGACAACAT | 660 |
| GATGGCTCAG AACCTGGTGG ACTTGCCGAT GTTTTCTGTC TACATGAGCA GTAACCCAGA | 720 |
| AGGTGGTGCG GGGAGCGAGC TGATTTTTGG AGGCTAGGAC CACTCCCATT TCTCTGGGAG | 780 |
| CCTGAATTGG GTCCCAGTCA CCAAGCAAGC TTACTGGCAC ATTGCACTGG ATAACATCCA | 840 |
| CGTGGGAGGC ACTGTTATGT TCTGCTCCGA GGGCTGCCAG GCCATTGTGG ACACAGGGAC | 900 |
| TTCCCTCATC ACTGGCCCTT CCGACAAGAT TAAGCAGCTG CAAAACGCCA TTGGGGCAGC | 960 |
| CCCCGTGGAT GGAGAATATG CTGTGGAGTG TGCCAACCTT AACGTCATGC CGGATGTCAC | 1020 |
| CTTCACCATT AACGGAGTCC CCTATACCCT CAGCCCAACT GCCTACACCC TACTGGACTT | 1080 |
| CGTGGATGGA ATGCAGTTCT GCAGCAGTGG CTTTCAAGGA CTTGACATCC ACCCTCCAGC | 1140 |
| TGGGCCCCTC TGGATCCTGG GGGATGTCTT CATTCGACAG TTTTACTCAG TCTTTGACCG | 1200 |
| TGGGAATAAC CGTGTGGGAC TGGCCCCAGC AGTCCCCTAA GGAGGGGCCT TGTGTCTGTG | 1260 |
| CCTGCCTGTC TGACAGACCT TGAATATGTT AGGCTGGGGC ATTCTTTACA CCTACAAAAA | 1320 |
| GTTATTTTCC AGAGAATGTA GCTGTTTCCA GGGTTGCAAC TTGAATTAAG ACCAAACAGA | 1380 |
| ACATGAGAAT ACACACACAC ACACACATAT ACACACACAC ACACTTCACA CATACACACC | 1440 |
| ACTCCCACCA CCGTCATGAT GGAGGAATTA CGTTATACAT TCATATTTTG TATTGATTTT | 1500 |
| TGATTATGAA AATCAAAAAT TTTCACATTT GATTATGAAA ATCTCCAAAC ATATGCACAA | 1560 |
| GCAGAGATCA TGGTATAATA AATCCCTTTG CAACTCCACT CAGCCCTGAC AACCCATCCA | 1620 |
| CACACGGCCA GGCCTGTTTA TCTACACTGC TGCCCACTCC TCTCTCCAGC TCCACATGCT | 1680 |
| GTACCTGGAT CATTCTGAAG CAAATTCCGA GCATTACAIC ATTTTGTCCA TAAATATTTC | 1740 |
| TAACATCCTT AAATATACAA TCGGAATTCA AGCATCTCCC ATTGTCCCAC AAATGTTTGG | 1800 |
| CTGTTTTTGT AGTTGGATTG TTTGTATTAG GATTCAAGCA AGGCCCATAT ATTGCATTTA | 1860 |
| TTTGAAATGT CTGTAAGTCT CTTTCCATCT ACAGAGTTTA GCACATTTGA ACGTTGCTGG | 1920 |
| TTGAAATCCC GAGGTGTCAT TTGACATGGT TCTCTGAACT TATCTTTCCT ATAAAATGGT | 1980 |
| AGTTAGATCT GGAGGTCTGA TTTTGTGGCA AAAATACTTC CTAGGTGGTG CTGGGTACTT | 2040 |
| CTTGTTGCAT CCTGTCAGGA GGCAGATAAT GCTGGTGCCT CTCTATTGGT AATGTTAAGA | 2100 |
| CTGCTGGGTG GGTTTGGAGT TCTTGGCTTT AATCATTCAT TACAAAGTTC AGCATTTT | |
| Seq ID NO: 54 Protein Sequence | |
| Protein Accession #: NP_001901.1 | |
| 1 11 21 31 41 51 | |
| \| \| \| \| \| \| | |
| MKTLLLLLLV GLELGEAQGS LNRVPLRRHP SLKKKLRARS QLSEFWKSNN LGMIQFTNSC | 60 |
| SMGQSAKEPL INYLGMEYFG TISIDSPPQN FTVIFGTGSS NLWVPSVYCT SPACKTNSRF | 120 |
| QPSQSSTYSQ PGQSFSIQYG TGSLSGIIDA GQVSVEGLTV VGQQFGESVT EPGQTFVGAE | 180 |
| FGGILGLGYP SLAVGGVTPV FDNMMAGNLV GLPMFSVYMS SNPEGGAGSE LIEGGYGNEN | 240 |
| FSGSLNWVPV TEQAYEQIAL GNIQVGGTVM FCSEGCQAIV GTGTSLITGP SGKIKQLQNA | 300 |
| IDAAPVGGEY AVECANLNVM PGVTFTINDV PYTLSPTAYT LLGPVGGNQF CSSGFQGLGI | 360 |
| NPPAGPLWIL GGVFINQFYS VFGRGNNRVG LAPAVP | |
| Seq ID NO: 55 DNA sequence | |
| Nucleic Acid Accession #: NM_018058.1 | |
| Coding sequence: 319 . . . 1575 | |
| 1 11 21 31 41 51 | |
| \| \| \| \| \| \| | |
| TACGCGCTGC GGGACCGGCA GGGGAACGCC ATCGGGGTCA CAGCCTGCGA CATCGACGGG | 60 |
| GACGGCCGGG AGGAGATCTA CTTCCTCAAC ACCAATAATG CCTTCTCGGG GGTGGCCACG | 120 |
| TACACCGACA AGTTGTTCAA GTTCCGCAAT AACCGGTGGG AAGACATCCT GAGCGATGAG | 180 |
| GTCAACGTGG CCCGTGGTGT GGCCAGCCTC TTTGCCGGAC GCTCTGTGGC CTGTGTGGAC | 240 |
| AGAAAGGGCT CTGGACGCTA CTCTATCTAC ATTGCCAATT ACGCCTACGG TAATGTGGGC | 300 |
| CCTGATGCCC TCATTGAAAT GGACCCTGAG GCCAGTGACC TCTCCCGGGG CATTCTGGCG | 360 |
| CTCAGAGATG TGGCTGCTGA GGCTGGGGTC AGCAAATATA CAGGGGGCCG AGGCGTCAGC | 420 |
| GTGGGCCCCA TCCTCAGCAG CAGTGCCTCG GATATCTTCT GCGACAATGA GAATGGGCCT | 480 |
| AACTTCCTTT TCCACAACCG GGGCGATGGC ACCTTTGTGG ACGCTGCGGC CAGTGCTGGT | 540 |
| GTGGACGACC CCCACCAGCA TGGGCGAGGT GTCGCCCTGG CTGACTTCAA CCGTGATGGC | 600 |
| AAAGTGGACA TCGTCTATGG CAACTGGAAT GGCCCCCACC GCCTCTATCT GCAAATGAGC | 660 |
| ACCCATGGGA AGGTCCGCTT CCGGGACATC GCCTCACCCA AGTTCTCCAT GCCCTCCCCT | 720 |
| GTCCGCACGG TCATCACCGC CGACTTTGAC AATGACCAGG AGCTGGAGAT CTTCTTCAAC | 780 |
| AACATTGCCT ACCGCAGCTC CTCAGCCAAC CCCCTCTTCC GCGTCATCCG TAGAGACCAC | 840 |
| GCAGACCCCC TCATCGAGGA GCTCAATCCC GGCGACGCCT TGGAGCCTGA GGGCCGGGGC | 900 |
| ACAGGGGGTG TGGTGACCGA CTTCGACGGA GAGGGGATGC TGGACCTCAT CTTGTCCCAT | 960 |
| GGAGAGTCCA TGGCTCAGCC GCTGTCCGTC TTCCGGGGCA ATCAGGGCTT CAACAACAAC | 1020 |
| TGGCTGCGAG TGGTGCCACG CACCCGGGTT GGGGCCTTTG CCAGGGGAGC TAAGGTCGTG | 1080 |
| CTCTACACCA AGAAGAGTGG GGCCCACCTG AGGATCATCG ACGGGGGCTC AGGCTACCTG | 1140 |
| TGTGAGATGG AGCCCGTGGC ACACTTTGGC CTGGGGAAGG ATGAAGCCAG CAGTGTGGAG | 1200 |
| GTGACGTGGC CAGATGGCAA GATGGTGAGC CGGAACGTGG CCAGCGGGGA GATGAACTCA | 1260 |
| GTGCTGGAGA TCCTCTACCC CCGGGATGAG GACACACTTC AGGACCCAGC CCCACTGGAG | 1320 |
| ACACCAATGA ATGCATCCAG TTCCCATTCG TGTGCCCTCG AGACAAGCCC GTATGTGTCA | 1380 |
| ACACCTATGG AAGCTACAGG TGCCGGACCA ACAAGAAGTG CAGTCGGGGC TACGAGCCCA | 1440 |
| ACGAGGATGG CACAGCCTGC GTGGCGACTC TCGGCCAGTC ACCGGGCCCC CGCCCCACCA | 1500 |
| CCCCCACCGC TGCTGCTGCC ACTGCCGCTG CTGCTGCCGC TGCTGGAGCT GCCACTGCTG | 1580 |
| CACCGGTCCT CGTACATGGA GATCTCAATC TGGGGTCGGT GGTTAAGGAG AGCTGCGAGC | 1620 |
| CCAGCTGCTG AGCACGGGTG GGACATGAAC CACCGGATGG AGTCCAGCAG GGGAGTGGGA | 1680 |
| AAGTGGGCTT GTGCTGCTGC CTAGACAGTA GGGATGTAAA GCCCTGGGAG CTAGACCCTC | 1740 |
| CCCAAGCCCA TCCATGCACA TTACTTAGCT AACAATTAGG GAGACTCGTA AGGCCAGGCC | 1800 |
| CTGTGCTGGG CACATAGCTG TGATCACAGC AGACAGGGTC GCTGCCCTGA TGGCGCTTAC | 1860 |
| ATTCCAGTGG GTCTAATGAC CATATCTTAG GACACAGATG TGCCCAGGGA GGTGGTGTCA | 1920 |
| CTGCACAGGA AGTATGAGGA CTTTAGTGTC CTGAGTTCAA ATCCTGATTC AGGAACTCAC | 1980 |
| AAAGCTATGT GACCTTACAC CAGTCACTTA ACTTGTTAGC CATCCATTAT CGCATCTGCA | 2040 |
| AAATGGGGAT TAAGAATAGA ATCTTGGGGT TAGTGTGGAG ATTAGATTAA ATGTATGTAA | 2100 |
| GACACTTGGC ACAAAACCTG GCACATAGTA AAGGCTCAAT AAAAACAAGT GCCTCTCACT | 2160 |
| GGGCTTTGTC AACACGTG | |
| Seq ID NO: 56 Protein Sequence | |
| Protein Accession #: NP_060528.1 | |
| 1 11 21 31 41 51 | |
| \| \| \| \| \| \| | |
| MGPEASGLSR GILALRGVAA EAGVSKYTGG RGVSVGPTLS SSASGIFCGN ENGPNFLFHN | 60 |
| RGGGTFVGAA ASAGVGGPHQ NGRGVALAGF NRGGKVGIVY GNWNGPNRLY LQNSTNGKVR | 120 |
| FRGIASPKFS MPSPVRTVIT AGFGNGQELE IFFNNIAYRS SSANRLERVI RRENGGPLIE | 180 |
| ELNPGGALEP EGRGTGGVVT GPGGGGMLGL ILSNGESMAC PLSVFRGNQG FNNNWLRVVP | 240 |
| RTRVGAPARG AKVVLYTKKS GANLRIIDGG SGYLCEMEPV ANFGLGKGEA SSVEVTWPGG | 300 |
| KMVSRNVASG EMNSVLEILY PRGEGTLQGP APLETPMNAS SSNSCALSTS PYVSTPMSAT | 360 |
| GAGPTRSAVG ATSPTRMACP AWGLSASNRA PAPPPPPLLL PLPLLLPLLE LPLLNRSS | |
| Seq ID NO: 57 DNA sequence | |
| Nucleic Acid Accession #: AJ279016.1 | |
| Coding sequence: 1 . . . 1962 | |
| 1 11 21 31 41 51 | |
| \| \| \| \| \| \| | |
| ATGTCCAGGA TGTTACCGTT CCTGCTGCTG CTCTGGTTTC TGCCCATCAC TGAGGGGTCC | 60 |
| CAGCGGGCTG AACCCATGTT CACTGCAGTC ACCAACTCAG TTCTGCCTCC TGACTATGAC | 120 |
| AGTAATCCCA CCCAGCTCAA CTATGGTGTG GCAGTTACTG ATGTGGACCA TGATGGGGAC | 180 |
| TTTGAGATCG TCGTGGCGGG GTACAATGGA CCCAACCTGG TTCTGAAGTA TGACCGGGCC | 240 |
| CAGAAGCGGC TGGTGAACAT CGCGGTCGAT GAGCGCAGCT CACCCTACTA CGCGCTGCGG | 300 |
| GACCGGCAGG GGAACGCCAT CGGGGTCACA GCCTGCGACA TCGACGGGGA CGGCCGGGAG | 360 |
| GAGATCTACT TCCTCAACAC CAATAATGCC TTCTCGGGGG TGGCCACGTA CACCGACAAG | 420 |
| TTGTTCAAGT TCCGCAATAA CCGGTGGGAA GACATCCTGA GCGATGAGGT CAACGTGGCC | 480 |
| CGTGGTGTGG CCAGCCTCTT TGCCGGACGC TCTGTGGCCT GTGTGGACAG AAAGGGCTCT | 540 |
| GGACGCTACT CTATCTACAT TGCCAATTAC GCCTACGGTA ATGTGGGCCC TGATGCCCTC | 600 |
| ATTGAAATGG ACCCTGAGGC CAGTGACCTC TCCCGGGGCA TTCTGGCGCT CAGAGATGTG | 660 |
| GCTGCTGAGG CTGGGGTCAG CAAATATACA GGGGGCCGAG GCGTCAGCGT GGGCCCCATC | 720 |
| CTCAGCAGCA GTGCCTCGGA TATCTTCTGC GACAATGAGA ATGGGCCTAA CTTCCTTTTC | 780 |
| CACAACCGGG GCGATGGCAC CTTTGTGGAC GCTGCGGCCA GTGCTGGTGT GGACGACCCC | 840 |
| CACCAGCATG GGCGAGGTGT CGCCCTGGCT GACTTCAACC GTGATGGCAA AGTGGACATC | 900 |
| GTCTATGGCA ACTGGAATGG CCCCCACCGC CTCTATCTGC AAATGAGCAC CCATGGGAAG | 960 |
| GTCCGCTTCC GGGACATCGC CTCACCCAAG TTCTCCATGC CCTCCCCTGT CCGCACGGTC | 1020 |
| ATCACCGCCG ACTTTGACAA TGACCAGGAG CTGGAGATCT TCTTCAACAA CATTGCCTAC | 1080 |
| CGCAGCTCCT CAGCCAACCG CCTCTTCCGC GTCATCCGTA GAGAGCACGG AGACCCCCTC | 1140 |
| ATCGAGGAGC TCAATCCCGG CGACGCCTTG GAGCCTGAGG GCCGGGGCAC AGGGGGTGTG | 1200 |
| GTGACCGACT TCGACGGAGA CGGGATGCTG GACCTCATCT TGTCCCATGG AGAGTCCATG | 1260 |
| GCTCAGCCGC TGTCCGTCTT CCGGGGCAAT CAGGGCTTCA ACAACAACTG GCTGCGAGTG | 1320 |
| GTGCCACGCA CCCGGTTTGG GGCCTTTGCC AGGGGAGCTA AGGTCGTGCT CTACACCAAG | 1380 |
| AAGAGTGGGG CCCACCTGAG GATCATCGAC GGGGGCTCAG GCTACCTGTG TGAGATGGAG | 1440 |
| CCCGTGGCAC ACTTTGGCCT GGGGAAGGAT GAAGCCAGCA GTGTGGAGGT GACGTGGCCA | 1500 |
| GATGGCAAGA TGGTGAGCCG GAACGTGGCC AGCGGGGAGA TGAACTCAGT GCTGGAGATC | 1560 |
| CTCTACCCCC GGGATGAGGA CACACTTCAG GACCCAGCCC CACTGGAGTG TGGCCAAGGA | 1620 |
| TTCTCCCAGC AGGAAAATGG CCATTGCATG GACACCAATG AATGCATCCA GTTCCCATTC | 1680 |
| GTGTGCCCTC GAGACAAGCC CGTATGTGTC AACACCTATG GAAGCTACAG GTGCCGGACC | 1740 |
| AACAAGAAGT GCAGTCGGGG CTACGAGCCC AACGAGGATG GCACAGCCTG CGTGGGGACT | 1800 |
| CTCGGCCAGT CACCGGGCCC CCGCCCCACC ACCCCCACCG CTGCTGCTGC CACTGCCGCT | 1860 |
| GCTGCTGCCG CTGCTGGAGC TGCCACTGCT GCACCGGTCC TCGTAGATGG AGATCTCAAT | 1920 |
| CTCCGGTCGG TGGTTAACGA GACCTGCGAG CCCAGCTGCT CAGCAGGGGT GGGACATGAA | 1980 |
| CCAGCGGATG GAGTCCAGCA GGGGAGTGGG AAAGTGGCCT TCTGCTGCTC CCTAGACAGT | 2040 |
| AGGGATGTAA AGGCCTGGGA GCTAGACCCT CCCCAAGCCC ATCCATGCAC ATTACTTAGC | 2100 |
| TAACAATTAG GGAGACTCGT AAGGCCAGGC CCTCTGCTGG GCACATAGCT GTGATCACAG | 2160 |
| CAGACAGGGT CGCTGCCCTG ATGGCGCTTA CATTCCAGTG CGTCTAATGA CCATATCTTA | 2220 |
| GGACACAGAT GTGCCCAGGG AGGTGGTGTC ACTGCACACG AACTATGAGG ACTTTAGTGT | 2280 |
| CCTGAGTTCA AATCCTGATT CAGGAACTCA CAAAGCTATG TGACCTTACA CCAGTCAGTT | 2340 |
| AACTTGTTAG CCATCCATTA TCGCATCTGC AAAATGGGGA TTAAGAATAG AATCTTGGGG | 2400 |
| TTAGTGTGGA GATTAGATTA AATGTATGTA AGACACTTGG CACAAAACCT GGCACATAGT | 2460 |
| AAAGGCTCAA TAAAAACAAG TGCCTCTCAC TGGGCTTTGT CAACACG | |
| Seq ID NO: 58 Protein Sequence | |
| Protein Accession #: CAC08451.1 | |
| 1 11 21 31 41 51 | |
| \| \| \| \| \| \| | |
| MSRMLPFLLL LWFLPITEGS QRAEPMFTAV TNSVLPPGYG SNPTQLNYGV AVTDVDHDGD | 60 |
| FEIVVAGYNG PNLVLKYGRA QKRLVNIAVG ERSSPYYALR GRQDNAIDVT ACGIDGGGRE | 120 |
| ETYFLNTNNA FSGVATYTGK LFKPRNNRNE GILSGEVNVA RGVASLFAGR SVACVGRKGS | 180 |
| GRYSTYTANY AYGNVGPGAL ISMGPEASGL SRGILALRGV AAEAGVSKYT GGRGVSVGPI | 240 |
| LSSSASGIFC CNENGPNFLF HNRGGGTFVG AAASAGVGGP NGNGRGVALA GFNRGGKVGI | 300 |
| VYGNWNGPHR LYLGMSTNGK VRFRGIASPK FSMPSPVRTV ITADFGNDQE LEIFPNNIAY | 360 |
| RSSSANRLFR VIRRENGGPL ISELNPGGAL EPEGRGTGGV VTGFGGGGML GLILSNGESM | 420 |
| AQPLSVFRGN QGFNNNNLRV VPRTRPGAFA RGAKVVLYTK ESGAMLRIID GGSGYLCEME | 480 |
| PVANFGLGKG EASSVEVTWP GGKNVSRNVA SGEMNSVLEI LYPRGEGTLQ GPAPLECGQG | 540 |
| FSQQENGNCN DTNECIQFPF VCPRGKPVCV NTYGSYRCRT NKKCSRGYEP NEGGTACVGT | 600 |
| LGQSPGPRPT TPTAAAATAA AAAAAGAATA APVLVGGGLN LGSVVKESCE PSC | |
| Seq ID NO: 59 DNA sequence | |
| Nucleic Acid Accession #: FGENESH | |
| Coding sequence: 1 . . . 4794 | |
| 1 11 21 31 41 51 | |
| \| \| \| \| \| \| | |
| ATGGCGTGTC CGGGAGGACT CCCAGCCCGT TGCTCTGGTT GGATGGGACT GGGTGGGCCC | 60 |
| AGCGGCTCCT CCCCAGCATC CCCTCCCCAT TCCTCCTCCA GGTACAATGG ACCCAACCTG | 120 |
| GTTCTGAAGT ATGACCGGGC CCAGAAGCGG CTGGTGAACA TCGCGGTCGA TGAGCGCAGC | 180 |
| TCACCCTACT ACGCGCTGCG GGACCGGGAG GGGAACGCCA TCGGGGTCAC AGCCTGCGAC | 240 |
| ATCGACGGGG ACGGCCGGGA GGAGATCTAC TTCCTCAACA CCAATAATGC CTTCTCGGGC | 300 |
| CACAGCAGCT CAGGGCACGT CCCTTCTGGG CTCCACAGAA ACAGGCCTGT GCTGAAGCCT | 360 |
| CCACCTACAA CCCCTGCAGG CCTCCTGGGT CTGCCTCCAC TCAGCGGAAG GGACTTTTCC | 420 |
| TCCTCCCTGG GTCAGGCTTC TCCGGACAGC AGGCAGGGAG AGAGGGTGCC GGTTCCCTGC | 480 |
| TGTCGGGGTG GACTGAGACC TACCCATGAA CCAGAACCAT TTCTTCTGAG ACCCAAATCA | 540 |
| GGGGTGGCCA CGTACACCGA CAAGTTGTTC AAGTTCCGCA ATAACCGGTG GGAAGACATC | 600 |
| CTGAGCGATG AGGTCAACGT GGCCCGTGGT GTGGCCAGCC TCTTTGCCGG ACGCTCTGTG | 660 |
| GCCTGTGTGG ACAGAAAGGG CTCTGGACGC TACTCTATCT ACATTGCCAA TTACGCCTAC | 720 |
| GGTAATGTGG GCCCTGATGC CCTCATTGAA ATGGACCCTG AGGCCAGTGA CCTCTCCCGG | 780 |
| GGCATTCTGG CGCTCAGAGA TGTGGCTGCT GAGGCTGGGG TCACCAAATA TACAGAAGGC | 840 |
| TTCTCCCACA CTGCCTCTCC AAGCATTGGT GAGATATCTG GCAGAACCGA GGAGCGGGAA | 900 |
| GGAGGAGACC CAGAGGAGGC AGATGAGGAG CACAGTGGGG ATGGAAGCAC CAGCCAACTG | 960 |
| TGCCGGCTGG GCTGGAAGGA CGGGCAGTTC AAGGAAGAAG CAGCAGCTTT GGTGGAGGAA | 1020 |
| CAGAGGGAGG CTGGGGCAGC TGGCGTGCCC AGAGGACGTG TTCGAACAGC TCTGCAGACT | 1080 |
| TCCAAAAGCC ATTTGGCTGA CAAGAACCTA TTTGGCCCAC CATGTTACTA TTCTGTCTGC | 1140 |
| GCGCCTTCTC CAGCCCACCC TTTCCCTGCC CGCCAAGCCC CCCAACACTA CCCTGTAGCC | 1200 |
| CCCCTTGTCA CTCAGCTAAT GACACATGGA CGTCTGGCTG GAAAACTAGC CCGGAGTGTC | 1260 |
| CCCCACCCCC GAGCCCCAGG AATGGACCCC AAATGTAAGG GCCGCCATGC TGAGCCCGGC | 1320 |
| CTGATGGCTG AGGCTTTGGG CGCGTGGCCA GCGCTCAGCA CCACTGTGGT GCCAGGGGGC | 1380 |
| CTGAGAAGCT GGGAGGAAAG CAGGCAGAAG GGGCAGGCCA TGTCCAGATG TGCACTCAGG | 1440 |
| GAGCTGGGAG GTCCCTGGAG CCAAGCCACA CAGCACCTGC CTGCTAGAGA GCTGTATGAC | 1500 |
| CTGGGAGAAC CTCCCATTTT ACAAAGAACA GACGGAGATC CAGGGAGGAG AAGGGACTCG | 1560 |
| CCCAAGGTCA CACAGGAGTG CCATCTAGTG GCCACCATGC CAGCTCTCGG GGGACTCGAG | 1620 |
| GGCCCCGGGA GGGTGGCCAA GCGAGAGATT GGGAGAGAGA CTGGGGCAGT AGGAAGACCA | 1680 |
| CTCTCCCATC CCCTGGTCCC CAACTTCCCC AGCTGCTTGA GGCCTCTTGA AGCCGGGACA | 1740 |
| GTGCCGGGAG CTGCCCTGCC TGGGAATCCT GGGAACTGGG TTCTGGACAT GGCCAAGGCC | 1800 |
| CTGGCGTGGA ACCAGATGGA AAAAGAGGAG GGGAAGATTC ATGGAGACCA TGAGCCCAGA | 1860 |
| TTTAGGCTCA GGAAAGCACG GGAAGCAGAA TTCCCCCCAG GCTCCTCTGA GGAGCCTCTG | 1920 |
| CTGCAGTTCC CCTCAGGCCT CAGAGGCAGC CCTGTCCTCC AGGTGGGCCT GGGGCTTGCT | 1980 |
| TCTGCCACTC ACTGTGGGTC GATGTCTTTT CTAGGGGGCC GAGGCGTCAG CGTGGGCCCC | 2040 |
| ATCCTCAGCA GCAGTGCCTC GGATATCTTC TGCGACAATG AGAATGGGCC TAACTTCCTT | 2100 |
| TTCCACAACC GGGGCGATGG CACCTTTGTG GACGCTGCGG CCAGTGCTGA ACGTCGTTTA | 2160 |
| GCCTTCATCG TTCACCTCAA ATATCACCTC TGCAGAGATT TTCCTCACTC CCTGTGCCAC | 2220 |
| CTAGCAGAAA CTGGTCCTTC CTCCTCCTGC TGCCCGTGGC ATGCACGTCT TCTTCAGGCT | 2280 |
| CCACATTGCC ATCATGGTTT GTCTATGAGC TTTACAAGGA CCGGGTCACG GTTCTATTCA | 2340 |
| TTCTTGACGC AAGGCTTGGC CTCCAGTGCC CACCGGAGGA CACTCAGCCT CCAGGGTTCT | 2400 |
| CAGGGGGCCC CACCCTGCCT TCTGGCAAGA GCTCCCTGTG TCCTGGGGTC TCTGATCCCC | 2460 |
| ACTGCCTATT ACATTGTCCT GTGGTCTGCC ATCCCAGAGA GCCTGATGAC CCACAGCTAT | 2520 |
| TTGTCCTCTG AAAGAGTCAA CGTGGGTGTG GACGACCCCC ACCAGCATGG GCGAGGTGTC | 2580 |
| GCCCTGGCTG ACTTCAACCG TGATGGCAAA GTGGACATCG TCTATGGCAA CTGGAATGGC | 2640 |
| CCCCACCGCC TCTATCTGCA AATGAGCACC CATGGGAAGG TCCGCTTCCG GGACATCGCC | 2700 |
| TCACCCAAGT TCTCCATGCC CTCCCCTGTC CGCACGGTCA TCACCGCCGA CTTTGACAAT | 2760 |
| GACCAGGAGC TGGAGATCTT CTTCAACAAC ATTGCCTACC GCAGCTCCTC AGCCAACCGC | 2820 |
| CTCTTCCGAT GCTCCATCCT GGCTCGTGGC TCTTCATCCT TGACAGCTGG TGGGAGGAAC | 2880 |
| GGTCAGGGAG AAGGTTTAAG AATCAGAAGG GGAGGGTTCC CAGGGCCAGG GGGTCAGGCC | 2940 |
| AAGGTCAACA CAGGTCCCCT GATGAAGAAA CAGAAAGGAA GGAAGGACGA GGACTGGGCA | 3000 |
| AGAGGCTGTG GGAATGCACG GCAAAGCCTG GCCAAGGAGC CGGCCTCTGC TATTGCAGGG | 3060 |
| AAAGGGAAGG GAAATGTGGC CCAAAGTGTG CCCAGAACCC AAGCGCCACA AGATACAAAG | 3120 |
| CCACACTACC ACAAAAAGGG GCTACAGGGT CCAATCACTA CCAGGAAAAG GGGCTACGGG | 3180 |
| GTCCAATCAC TACCAGGAAA AGGGGCTACG GGGTCCAATC ACTACCAGGA AAAGGGGCTA | 3240 |
| CGGGGTCCAA TCACTACCAG GAAAAGGGGC TACCGCCTCC AATCACTACC AGGAAAAGGG | 3300 |
| GCTACGGGCT CCAATCACTA CCAGGAAAAG GGGCTACACG CTCCAATCAC TACCAGGAAA | 3360 |
| AGGGGCTACG GGCTCCAATC ACTACCAGGA AAAGGGGCTA CACGGTCCAA TCACTACCAC | 3420 |
| AGAAAGGGGC TACGGGCTCC AATCACTACC AGGAAAAGGG GCTACGGGCT CCAATCACTA | 3480 |
| CCAGGAAAAG GGGCTACAGG GTCCAATCAC TACCAGGAAA AGGGCCTACC CGGTCCAATC | 3540 |
| ACTACCAGGA AAAGGGGCTA CGGGCTCCAA TCACTACCAG GAAAAGGGCC TACCGGGTCC | 3600 |
| AATCACTACC AGGAAAAGGG GCTACAGGGT CCAATCACTA CCAGGAAAAG CGGCTACAGG | 3660 |
| GTCCAATCAC TACCACAGAA AGGGGCTACG GGCTCCAATC ACTACCAGGA AAACGGGCTA | 3720 |
| CGGGGTCCAA TCACTACCAG GAAAAGGGGC TACGGGCTCC AATCACTACC AGGAAAAGAG | 3780 |
| GCTATGGGGT CCAATCACTA CCAGGAAAAG GGGCTACGGG CTCCAATCAC TACCAGGAAA | 3840 |
| AGGGGCTATG GGGTCCAATC ACTACCACAG AAAGGGGCTA CGGGGTCCAA CGTCATCCGT | 3900 |
| AGAGAGCACG GAGACCCCCT CATCGAGGAG CTCAATCCCG GCGACGCCTT GGAGCCTGAG | 3980 |
| GGCCGGGGCA CAGGGGGTGT GGTGACCGAC TTCGACGGAG ACGGGATGCT GGACCTCATC | 4020 |
| TTGTCCCATG GAGAGTCCAT GGCTCAGCCG CTGTCCGTCT TCCGGGGCAA TCAGGGCTTC | 4080 |
| AACAACAACT GGCTGCGAGT GGTGCCACGC ACCCGGTTTG GCGCCTTTGC CAGGGGAGCT | 4140 |
| AAGGTCGTGC TCTACACCAA GAAGAGTGGG GCCCACCTGA GGATCATCGA CGGGGGCTCA | 4200 |
| GGCTACCTGT GTGAGATGGA GCCCGTGGCA CACTTTGGCC TGGGGAAGGA TGAAGCCAGC | 4260 |
| AGTGTGGAGG TGACGTGGCC AGATGGCAAG ATGGTGAGCC GGAACGTGGC CACCGGGGAG | 4320 |
| ATGAACTCAG TGCTGGAGAT CCTCTACCCC CGGGATGAGG ACACACTTCA GGACCCAGCC | 4380 |
| CCACTGGAGT GTGGCCAAGG ATTCTCCCAG CAGGAAAATG GCCATTGCAT GGACACCAAT | 4440 |
| GAATGCATCC AGTTCCCATT CGTGTGCCCT CGAGACAAGC CCGTATGTGT CAACACCTAT | 4500 |
| GGAAGCTACA GGTGCCGGAC CAACAAGAAG TGCAGTCGGG GCTACGAGCC CAACGAGGAT | 4580 |
| GGCACAGCCT GCGTGGGIAC TGAGCTAGGC TCTAGGCATA CAATGACGTG GAAACCAAGG | 4820 |
| CCCAAAAAGG AGCTGCAACT TTCCCAAGGC ATCTGCACCC CCGTCTGGTC CTTTTTCCTG | 4680 |
| CCGGGTTGCC GGCTGCTCCT CAAAAGAGCT CAGCTCCAGG CTGCTCCCAG CACCCTTCTC | 4740 |
| CAGAAAGCTC CAGGTATTCC AGAAGCCCAA GTGTATGAAC AAGATCAGGA ATAA | |
| Seq ID NO: 60 Protein Sequence | |
| Protein Accession #: FGENESH | |
| 1 11 21 31 41 51 | |
| \| \| \| \| \| \| | |
| MACPGGLPAR CSGWMGLGCP SGSSPASPPN SSSRYNGPNL VLKYGRAQKR LVNIAVGERS | 60 |
| SPYYALRGRQ DNAIDVTACG IDGGGREEIY FLNTNNAFSG HSSSAQVPSG LNRNRPVLKP | 120 |
| PPTTPAGLLG LPPLSGRGFS SSLGQASPGS RQGERVPVPC CRGGLRPTHE PEPFLLRPKS | 180 |
| GVATYTGKLF KEENNEWEGI LSGEVNVARG VASLFAGRSV ACVGRKGSGR YSIYIANYAY | 240 |
| GNVGPGALIE MGPEASGLSR GILALRGVAA EAGVSKYTEG FSNTASPSID EISGRTEERE | 300 |
| GGGPEEAGEE HSGGGSTSQL CRLGWKGGGF KEEAAALVEE QREAGAAGVP RGRVRTALQT | 360 |
| SKSNLAGKNL FGPPCYYSVC APEPARPEPA RQAPQNYPVA PLVTQLMTHG RLAGKLARSV | 420 |
| PHPRAPGMGP KCKGRNAEPG LMAEALGAWP ALSTTVVPGG LRSWEESRQK GQANSRCALR | 480 |
| ELGGPWSQAT QNLPARELYG LGEPPILQRT GGGPGRRRGS PKVTQECNLV ATEPALGGLE | 540 |
| GPGRVAEREI GRETGAVGRP LSNPLVPNFP SCLRPLEAGT VPGAALPGNP GNNVLGMAEA | 600 |
| LAWNGMEKEE GKIHGGHEPR FRLRKAREAE FPPGSSEEPL LQFPSGLRGS PVLGVGLGLA | 660 |
| SATNCGSMSF LGGRGVSVGP ILSSSASGIF CGNENGPNFL FNNRGGGTFV GAAASAERRL | 720 |
| AFIVNLKYHL CRGFPHSLCH LAETGPSSSC CPWNARLLQA PHCHHGLSMS FTRTGSRFYS | 780 |
| FLTQGLASSA HRRTLSLQGS QGAPPCLLAR APCVLGSLIP TAYYIVLWSA IPESLMTNSY | 840 |
| LSSERVNVGV GGFPQNDRGV ALAGENEGGE VGIVYGNWNG PHRLYLQMST NDEVEFEGIA | 900 |
| SPKFSMPSPV RTVITAGFGN GQELEIFFNN IAYRSSSANR LFRCSILARG SSSLTAGGRN | 960 |
| GGGEGLKIER GGFPGPGGGA KVNTGPLMKK QEGREGEGWA RGCDNAGQSL AKEPASAIAG | 1020 |
| KGKGNVAQSV PRTQAPQGTK PNYNKKGLQG PITTRKRGYG VQSLPGKGAT GSNNYQEKGL | 1080 |
| RGPITTRKRG YGVQSLPGKG ATGSNNYQEK GLQGPITTRK RGYGLQSLPG KGATGSNNYH | 1140 |
| RKGLRAPITT RKRGYGVQSL PGKGATGSNN YQEKGLRGPI TTRKRGYGLQ SLPGKGATGS | 1200 |
| NHYQEKGLQG PITTEKEGYR VQSLPQKGAT GSNNYQEKGL RGPITTRKRG YGLQSLPGKE | 1280 |
| AMGSNNYQEK GLRAPITTRK RGYGVQSLPQ KGATGSNVIR REHGDPLIEB LNPGGALEPE | 1320 |
| GRGTGGVVTG FGGGGMLGLI LSNGESMAQP LSVFRGNQGF NNNWLRVVPR TEFGAFARGA | 1380 |
| KVVLYTKKSG AELKIIDGGS GYLCEMEPVA NFGLGKGEAS SVEVTWPGGK MVSRNVASGE | 1440 |
| MNSVLEILYP RGEGTLQGPA PLECGQGFSQ QENGNCNGTN ECIQFPFVCP RGKPVCVNTY | 1500 |
| GSYRCRTNKK CSRGYEPNEG GTACVGTELG SRNTMTWKPR PKKELQLSQG ICTPVWSFFL | 1560 |
| PGCRLLLKRA QLQAAPSTLL QKAPGIPEAQ VYEQGQE | |
| Seq ID NO: 61 DNA sequence | |
| Nucleic Acid Accession #: NM_000584.1 | |
| Coding sequence: 75 . . . 374 | |
| 1 11 21 31 41 51 | |
| \| \| \| \| \| \| | |
| AGCAGAGCAC ACAAGCTTCT AGGACAAGAG CCAGGAAGAA ACCACCGGAA GGAACCATCT | 60 |
| CACTGTGTGT AAACATGACT TCCAACCTGG CCGTGGCTCT CTTCCCACCC TTCCTGATTT | 120 |
| CTGCAGCTCT GTGTGAAGGT GCAGTTTTCC CAACGAGTGC TAAAGAACTT AGATGTCAGT | 180 |
| GCATAAACAC ATACTCCAAA CCTTTCCACC CCAAATTTAT CAAAGAACTG AGAGTGATTG | 240 |
| AGAGTCGACC ACACTGCGCC AACACACAAA TTATTCTAAA GCTTTCTGAT GGAAGAGAGC | 300 |
| TCTGTCTGCA CCCCAACGAA AACTGGCTCC ASASGUTTGT GGAGAAGTTT TTGAAGAGGG | 360 |
| CTGAGAATTC ATAAAAAAAT TCATTCTCTG TGGTATCCAA GAATCAGTGA AGATGCCAGT | 420 |
| GAAACTTCAA GCAAATCTAC TTCAACACTT CATGTATTGT GTGGGTCTGT TGTAGGGTTG | 480 |
| CCAGATGCAA TACAAGATTC CTGGTTAAAT TTGAATTTCA GTAAACAATG AATAGTTTTT | 540 |
| CATTGTACCA TCAAATATCC AGAACATACT TATATGTAAA GTATTATTTA TTTGAATCTA | 600 |
| CAAAAAACAA CAAATAATTT TTAAATATAA GGATTTTCCT AGATATTGCA CGGGAGAATA | 660 |
| TACAAATAGC AAAATTGAGC CAAGGGCCAA GAGAATATCC GAACTTTAAT TTCAGGAATT | 720 |
| GAATGGGTTT GCTAGAATGT GATATTTGAA GCATCACATA AAAATGATGG GACAATAAAT | 780 |
| TTTGCCATAA AGTCAAATTT AGCTGGAAAT CCTGGATTTT TTTCTGTTAA ATCTGGCAAC | 840 |
| CCTAGTCTGC TAGCCAGGAT CCACAAGTCC TTGTTCCACT GTGCCTTGGT TTCTCCTTTA | 900 |
| TTTCTAACTG GAAAAAGTAT TAGCCACCAT CTTACCTCAC AGTGATGTTG TGACGACATG | 960 |
| TGGAAGCACT TTAAGTTTTT TCATCATAAC ATAAATTATT TTCAAGTGTA ACTTATTAAC | 1020 |
| CTATTTATTA TTTATGTATT TATTTAAGCA TCAAATATTT GTGCAAGAAT TTGGAAAAAT | 1080 |
| AGAAGATGAA TCATTGATTG AATAGTTATA AAGATGTTAT AGTAAATTTA TTTTATTTTA | 1140 |
| GATATTAAAT GATGTTTTAT TAGATAAATT TCAATCAGGG TTTTTAGATT AAACAAAGAA | 1200 |
| ACAATTGGGT ACCCAGTTAA ATTTTCATTT CAGATAAACA ACAAATAATT TTTTAGTATA | 1260 |
| AGTACATTAT TGTTTATCTG AAAGTTTTAA TTGAACTAAC AATCCTAGTT TGATACTCCC | 1320 |
| AGTCTTGTCA TTGCCAGCTC TGTTGGTAGT GCTGTGTTGA ATTACGGAAT AATGAGTTAG | 1360 |
| AACTATTAAA ACAGCCAAAA CTCCACAGTC AATATTAGTA ATTTCTTGCT GGTTGAAACT | 1440 |
| TGTTTATTAT GTACAAATAG ATTCTTATAA TATTATTTAA ATGACTGCAT TTTTAAATAC | 1500 |
| AAGGCTTTAT ATTTTTAACT TTAAGATGTT TTTATGTGCT CTCCAAATTT TTTTTACTGT | 1560 |
| TTCTGATTGT ATCGAAATAT AAAAGTAAAT ATGAAACATT TAAAATATAA TTTGTTGTCA | 1620 |
| AAGTAAAAAA AAAAAAAAA | |
| Seq ID NO: 62 Protein Sequence | |
| Protein Accession #: NP_000575.1 | |
| 1 11 21 31 41 51 | |
| \| \| \| \| \| \| | |
| MTSKLAVALL AAFLISAALC EGAVLPRSAK ELRCQCIKTY SKPFNPKFIK ELRVIESGPN | 60 |
| CANTEIIVKL SGGRELCLGP KENWVQRVVE KFLKRAENS | |
| Seq ID NO: 63 DNA sequence | |
| Nucleic Acid Accession #: EOS sequence | |
| Coding sequence: 52-1146 | |
| 1 11 21 31 41 51 | |
| \| \| \| \| \| \| | |
| GTTACCCACC ATTGTGACTG ACACAGCCTG GATCTGAACG CTGATCCCAT AATGCATCCT | 60 |
| CAACTCGTCA TCTTAACCCT CATCCTACAT CTGGCAGATT CTGTAGCTGG TTCTGTAAAG | 120 |
| GTTGGTGGAG AGGCAGGTCC ATCTGTCACA CTACCCTGCC ACTACAGTGG AGCTGTCACA | 180 |
| TCCATGTGCT GGAATAGAGG CTCATGTTCT CTATTCACAT GCCAAAATGG CATTGTCTGG | 240 |
| ACCAATGGAA CCCACGTCAC CTATCGGAAG GACACACGCT ATAAGCTATT GGGGGACCTT | 300 |
| TCAAGAAGGG ATGTCTCTTT GACCATAGAA AATACAGCTG TGTCTGACAG TGGCGTATAT | 360 |
| TGTTGCCGTG TTGAGCACCC TGGGTGGTTC AATGACATGA AAATCACCGT ATCATTGGAG | 420 |
| ATTGTGCCAC CCAAGGTCAC GACTACTCCA ATTGTCACAA CTGTTCCAAC CGTCACGACT | 480 |
| GTTCGAACGA GCACCACTGT TCCAACGACA ACGACTGTTC CAATGACGAC TGTTCCAACG | 540 |
| ACAACTGTTC CAACAACAAT GAGCATTCCA ACGACAACGA CTGTTCTGAC GACAATGACT | 600 |
| GTTTCAACGA CAACGAGCGT TCCAACGACA ACGAGCATTC CAACAACAAC AAGTGTTCCA | 660 |
| GTGACAACAA CTGTCTCTAC CTTTGTTCCT CCAATGCCTT TGCCCAGGCA GAACCATGAA | 720 |
| CCAGTAGCCA CTTCACCATC TTCACCTCAG CCAGCAGAAA CCCACCCTAC GACACTGCAG | 780 |
| GGAGCAATAA GGAGAGAACC CACCAGCTCA CCATTGTACT CTTACACAAC AGATGGGAAT | 840 |
| GACACCGTGA CAGAGTCTTC AGATGGCCTT TGGAATAACA ATCAAACTCA ACTGTTCCTA | 900 |
| GAACATAGTC TACTGACGGC CAATACCACT AAAGGAATCT ATGCTGGAGT CTGTATTTCT | 960 |
| GTCTTGGTGC TTCTTGCTCT TTTGGGTGTC ATCATTGCCA AAAAGTATTT CTTCAAAAAG | 1020 |
| GAGGTTCAAC AACTAAGTGT TTCATTTAGC AGCCTTCAAA TTAAAGCTTT GCAAAATGCA | 1080 |
| GTTGAAAAGG AAGTCCAAGC AGAAGACAAT ATCTACATTG AGAATAGTCT TTATGCCACG | 1140 |
| GACTAAGACC CAGTGGTGCT CTTTGAGAGT TTACGCCCAT GAGTGCAGAA GACTGAACAG | 1200 |
| ACATGACCAC ATCAGACGTC TTTTAGACCC CAAGACAATT TTTCTGTTTC AGTTTCATCT | 1260 |
| GGCATTCCAA CATGTCAGTG ATACTGGGTA GAGTAACTCT CTCACTCCAA ACTGTGTATA | 1320 |
| GTCAACCTCA TCATTAATGT AGTCCTAATT TTTTATGCTA AAACTGGCTC AATCCTTCTG | 1380 |
| ATCATTGCAG TTTTCTCTCA AATATGAACA CTTTATAATT GTATGTTCTT TTTAGACCCC | 1440 |
| ATAAATCCTG TATACATCAA AGAGAATAGT CCCTGGAAAC ATAGCAAATG AACTTCTATC | 1500 |
| TTGGCCATCA CAGCTGTCCA GAAGAGGGGA ATCTGTCTTA AAAACCAGCA AATCCAACGT | 1560 |
| GAGACTTCAT TTGGAAGCAT TGTATGATTA TCTCTTGTTT CTATGTTATA CTTCCAAATG | 1620 |
| TTGCATTTCC TATGTTTTCC AAAGGTTTCA AATCGTGGGT TTTTATTTCC TCCGTGGGGA | 1680 |
| AACAAAGTGA GTCTAACTCA CAGGTTTAGC TGTTTTCTCA TAACTCTGGA AATGTGATGC | 1740 |
| ATTAAGTACT GGATCTCTGA ATTGGGCTAG CTGTTTTACC AGTTAAAGAG CCTACAATAG | 1800 |
| TATGGAACAC ATAGACACCA GGGGAAGAAA ATCATTTGCC AGGTGATTTA ACATATTTAT | 1860 |
| GCAATTTTTT TTTTTTTTTT TGAGATUGAG CTTTGCTCTT GTTGCCCAGG CTGGAGTGCG | 1920 |
| ATGGTGAAAT CTCGGCTCAC TGTAACCTCC ACCTTCCGGG TTCAAGCAAT TCTCCCGCCT | 1980 |
| CAGCCTCACG AGTAGCTGGG ATTACAGGTG TCCGCCACTA TGCTCAGCTA ATTTTTTGTA | 2040 |
| TTTTTTTAGT AGAGACAGGG TTTCATCATG TTGGCCAGAA TGATCTCGAA CTCCTGACCT | 2100 |
| CAGAGGATCC AGCCTCCTTG GCCTTCCAAA GTGCTGGGAT TACAGGCATG AGCCACCGTG | 2160 |
| CCTGGTCCAT TTATGCAATT TTTATTACAC GTGTGTCATG GTGCTAAGCA TTTAAACAAC | 2220 |
| CATCAGGCTT TCTGAACAAA TTTTTAGAAC CAAGAAGATC AGCAATAAAA GAAAAAAAAA | 2280 |
| AGGCGGGAGT AGGGAAAAGT CTGCCTTTTC TGCCTTATAA GTTACATTGC AGATATAAGT | 2340 |
| GTTTGAAACT CACTAGTTTT GACCCAAATT CTAAATCGCC TCCAAACCAC TTTCATCTTT | 2400 |
| AAAATTGTTT AGGAGGCTAG CGCCGTGGCT CATGCCTGTA ATCCCAGCAC TTTGTGAAGC | 2460 |
| CAAGGTGGGC AGGTAGCTTG AGCCCAGAAG TTTGAGACCA GCCTGGGCAA CATGGCAATA | 2520 |
| GCCCATCTCT ATTAAGAAAA AAATTTAAGG AAAAAAAAAA AAGATAAAAT TGTTTTGGAA | 2580 |
| AGGCAAAGTG TCTTTCTCAT ATCTAATCAA TTCCTGTATT TTTTTTTTTT TTTTTTTTAC | 2640 |
| AATTCAATTA GGCTTATTGT AAGGATATCT TTAGTCAGTA TAAAAATATA TAAAAAACAA | 2700 |
| TTATACTAAA CCAGTATAAT TGTTTAGAAG TATATATTTG CCGGCCGGGC ATGGTGGCTC | 2760 |
| ATGCCTGTAA TCCCAGGGCT TTGGAAGGCT GAGGTGAGTG GATCACTAGA GGTCGGGAGT | 2820 |
| TTGAGACCAG CCTGACCAAC ATGGAGAAAC ACTGTTCTCT ACTAAGTATA GATTTGCAAG | 2880 |
| GGCTAGATTT GTCAGAAAAG AAACATTTCT ACATGAACAG TTAAGGCTGG GTATTAATTT | 2940 |
| CAATTTCAGT TTGAGTAAAC TCTGAAATAA TTCATGGTTG AGGTTCAGAG CTACCCATAC | 3000 |
| CATAGTTGTC TGGGGCAACA GGATGCTTGC TTTCAGAGGA AACTTTTTGT TTTTGTTTTT | 3060 |
| GTTTTTTTGA GATGGAGTTT TGCTCTTGTC ACCCAGGCTG AAATGCAATG GCACAATCTT | 3120 |
| GGCTCACTGC AACCTCCACC TCCCAGGTTC AAGTGATTCT CCTGCCTCAG CCTCCTGAGT | 3180 |
| AGCTGGGATT ACAGATGCCC GCCACAACGC CCAGCTTATT TTTAGTAGAG ACAGGGTTTC | 3240 |
| ACCATATTGG CCAGGCTGGC CTCCAACTCC TGACCTCAGG TGAGCCACCC GCTTTGGCCT | 3300 |
| CCCCAAAGTG TTGGGATTAC AGGCATAAGC CACCGCGCCC AGCCAGAGGC AACATTTTTT | 3360 |
| AACGCAGTTA TCATTCTAGG AAATTTATAG GTCCTTTGAA GGAAAATTCT GTGGGCAAAT | 3420 |
| AAGATTGTGA TACATGCTAT TTCAGTTTTC CCAAATGTGG CCAGCCCGAT CTGGTCAAAA | 3480 |
| ATTTTATTTT TTAAAAGCTA TAGTGTCTTT TTTTCTTAAA TTTGAGGCAA CATGCACAAA | 3540 |
| ATTGGAGATT TGAAATTAAA GCCAAGATTT GTAGTTTCTC TGGAAAGACC TGGCAAGATT | 3600 |
| GGACTGGATT GCTATGTGAC CAGGGTCCCA CTAGATGGGG CTGCATCCTC TAATCCCCAA | 3660 |
| ATCCTTATGT TCCCTGCATG CTCACCTTTG TTACCTGCCT GACACCICTG GGGCTTTTAA | 3720 |
| CTTTATGGCA ACTGCCCTAT TCTCTGGATC CTTCCTGAGG ATTTATGATG CGTAATACTC | 3780 |
| CAGGAATCTG GTTAGCTTTG CTTAACACAT TTCCAAAACT TGTTTGAATG CATGAGTACA | 3640 |
| GTCACTAGTA GCATTCTGTG CAGTACAATG TATGGGGGCT TAGGAGTTTA GGGTAGTATA | 3900 |
| CAGGATTAGG GATAGCACTT GAGTCTAATC CTAACTCTTA GCAGTTACAC TGGATGACAT | 3960 |
| TAGAGCAAAT GGTTCTTTAC GTCTACATTT TCTTCATCTG TAGATGTAAT AATTTCCATA | 4020 |
| TCAACTATGA TGTACACTGC TAATTCCAAT GAAATGTTAC ATGTGAGAAG TCTTTGAAAT | 4080 |
| GTAAAAAACA CTACAGATAC TGAAGCAGTT TGGAGAATTA AAAAACACTA CGAAAACACA | 4140 |
| GCTTGGTAAA TAATTTTTTT TTTCTTTTGA GACAGAGTCT GGCTCTGTCA TCCAGGCTAG | 4200 |
| AGTGTAGTG | |
| Seq ID NO: 64 Protein sequence: | |
| Protein Accession #: NP_036338.1 | |
| 1 11 21 31 41 51 | |
| \| \| \| \| \| \| | |
| MNPQVVILSL ILHLAGSVAG SVKVGGEAGP SVTLPCNYSG AVTSMCWNRG SCSLFTCQNG 60 | |
| IVWTNGTNVT YRKGTRYKLL GGLSRRGVSL TIENTAVSGS GVYCCRVENR GWFNGMKITV 120 | |
| SLEIVPPKVT TTPIVTTVPT VTTVRTSTTV PTTTTVPTTT VPTTMSIPTT TTVPTTMTVS 180 | |
| TTTSVPTTTS IPTTTSVPVT TTVSTFVPPM PLPRQNHEPV ATSPSSPQPA ETNPTTLQGA 240 | |
| IRREPTSSPL YSYTTGGNGT VTESSGGLWN NNQTQLFLEN SLLTANTTKG IYAGVCISVL 300 | |
| VLLALLGVII AKKYFFKKEV QQLSVSFSSL QIKALQNAVE KEVQAEGNIY IENSLYATG | |
| Seq ID NO: 65 DNA sequence | |
| Nucleic Acid Accession #: EOS sequence | |
| Coding sequence: 2932-4764 | |
| 1 11 21 31 41 51 | |
| \| \| \| \| \| \| | |
| AGAAGAAAGC TGGTAGGGGC TGGGAGAGGG TACCACAGGG GAGTATGATC TACTTGGGGG | 60 |
| CCAGAGAAGG TTCCCTGAGG AAATAGTACC TGAACTTAGA CTTGAAGGAT AACAGATGTT | 120 |
| AACTGGGAGG AGAGAATGTT CCAGGCAGAG GAAAAGGCAT ATGCAAAAGT CCAGCGCCTT | 180 |
| GAAGGAGCAC AGCTGGGGTG CCTGGAGTGA GATGGAGCTG GAAAGATCCA GGTGGAGCTT | 240 |
| GACCTGAAGT ACCAGCCCCC AGAGGGCGCT ACTGGAGCCT GGTCAGAGGA GGACTTTGGG | 300 |
| GCACCCATCC AGGACAGCTT CGAGTTAATC ATCCCCAATG TGGGCTTCCA GGAACTGGAG | 360 |
| CCTGGGGAGG CCCAGCTGGA GCGGCGGGCA GTGGCTCTAG GCCGCAGGCT AGCTCGAAGT | 420 |
| CTAGGCCAGC AGGACGATGA AGAGAATGAG CTGGAGCTTG AGCTGGAGCA GGACCTGGAT | 480 |
| GATGAGCCTG ACGTGGAACT TTCTGGTGTT ATGTTCAGCC CCCTCAAGAG CCGCGCCAGG | 540 |
| GCCCTGGCCC ATGGGGATCC CTTCCAGGTG TCCAGAGCTC AAGACTTCCA GGTGGGAGTC | 600 |
| ACTGTGCTGG AAGCCCAGAA ACTGGTGGGA GTCAACATTA ACCCCTATGT GGCCGTGCAA | 660 |
| GTGGGGGGGC AGCGCCGTGT GACCGCCACA CAGCGTGGGA CCAGTTGCCC CTTCTACAAT | 720 |
| GAGTACTTCT TGTTCGAATT TCATGACACG CGGCTTCGTC TCCAAGACTT GCTGCTGGAG | 780 |
| ATCACGGTGA GTGGGGTAGG GGTGACCAGT GTCCTTCAGA GAAGGGGGGA TGAGAAAGCT | 840 |
| GCAGGACTAA CACCACCTTC CCCCAAGGCT TTCCATTCGC AGACCCTCCC CTTTATGGCC | 900 |
| ACCCGGATAG GCACCTTCAG GATGGACCTG GGCATCATCT TGGACCAGCC AGGTATGGAA | 960 |
| TCGTCCCCTT ATTGAGACTC TGCACGGACA AGGGCCCTAG AGATTGACCC TGCAGTGACT | 1020 |
| CCGCATGGAC CCCTATACAC TCACTTCGGA GAGGGCCATC TCTGGCGGAG GCTGAACTCT | 1080 |
| TGGCACTTCC GCCCCTCCCT GCTGAGCCAG AGAAGCCCTG GCCATTGTCC GTCACTCCGA | 1140 |
| TAGCCTCACG GCCACCCTGT GCGTCCCGCC GGTCGCCCCT TACCCCTGGC TCGCCCCTTC | 1200 |
| GCCCTTAGAT GGCCAGTTCT ACCAAAGATG GGTTCCGCTG CATGATCCCC GAGACACCCG | 1260 |
| CGCCGGGACC AAGGGTTTCA TTAAGGTCAC CTTGTCCGTG AGGGCGCGCG GGGACCTGCC | 1320 |
| CCCTCCAATG CTACCCCCGG CCCCAGGGCA CTGTTCGGAC ATCGAGAAGT GAGCCGGGGT | 1380 |
| GAGGTGGGGA GGAGGACATG GATCCGGGGG TGGCCGTGGG GCGCGGATAA GGGGAGGGGC | 1440 |
| CGAGATCCCA GTTTCTCCCC CCCCGCTCGG TGCCCCCTCC CCTAGGAACC TGCTCCTGCC | 1500 |
| GCGCGGGGTG CCCGCCGAGA GGCCATGGGC GCGGCTCCGC GTGCGCCTGT ACCGCGCCGA | 1560 |
| GGCGCTTCCC GCGCTGCGCC TGGGGCTGCT GGGCAGGCTG GTCCGCGCCC TGCACGACCA | 1620 |
| GCGCGTCCTG GTGGAGCCCT ATGTGCGGGT GTCTTTCCTG GGGCAGGAGG GCGAGACGTC | 1680 |
| GGTGAGCGCC GAGGCGGCGG CGCCCGAATG GAACGAGCAG CTGAGCTTGG TGGAGCTCTT | 1740 |
| CCCGCCGCTG ACGCGCAGCC TCCGCCTGCA GCTGCGGGAC GACGCGCCCC TGGTCGACGC | 1800 |
| GGCACTCGCT ACGCACGTGC CGGACCTGAG GCGGATCTCC CATCCGGGCC GCGCGGCGGG | 1860 |
| GTTTAACCCT ACCTTCGGCC CGGCCTGGGT GCCCCTCTAT GGCTCGCCCC CCGGCGCGGG | 1920 |
| GCTCCGGGAT AGTCTTCAAG GTCTCAACGA AGGCGTTGGC CAAGGCATTT GGTTCCGCGG | 1980 |
| CCGCCTTCTG CTGGCTGTGT CCATGCAGGT GTTGGAAGGG AGACCTGAAC CTGAGCCTCC | 2040 |
| CCAGGCCCAC CAGCGGTCCA CGTTGTCCCG GCTCACCCGA AAGAAGAAAA AGAAAGCCAG | 2100 |
| AAGGGATCAG ACCCCAAAGG CGGTTCCGCA GCACTTGGAC GCCAGCCCCC GTGCCGAGGG | 2160 |
| GCCTGAGATC CCCCGTGCCA TGCAGGTGGA GGTGGAGGAG CTGCTGCCGC TGCCAGAGAA | 2220 |
| TGTCCTGGCG CCCTGTGAAG ATTTCCTGCT TTTCCGTGTG CTCTTCGAGG CCACCATGAT | 2280 |
| CGACCCCACC GTGGCCTCCC AGCCCATCAG CTTCGAGATC TCCATTGGTG TGTGGCCTAG | 2340 |
| CCGAACCCCT GAGTGCCATT TCAGACCTTA GAACCCTGGA AGGGGTGTTG ACTTTCAGTC | 2400 |
| GCGCAGGCCG TCTGGAGGAG CAATTGGGCC GAGGGTCCAG GGCTGGGGAG GGAACTGAGG | 2460 |
| GTGCAGCCGT GGAGGCTGAG CCTCTGCTGG GAGCCAGGCC AGAGGAGGAG AAAGAGGAGG | 2520 |
| AAGAACTGGG GACCCATGCT CAGCGGCCTG AGCCCATGGA CGGCAGTCGG CCATACTTCT | 2580 |
| GCTTGCCCCT CTGTCACTGC AAGCCATGGA TGCATGTGTG GAGTTGCTGG GAGGACCACA | 2640 |
| CCTGGCGCCT GCAGAGCACC AACTGCGTGC GCAAAGTGGC CGAGAGCCTG GACCAGGGGC | 2700 |
| TGCAGGAGGT TGAGAGACTG CAGCGCAAGC CGGGGCCTGG CGCCTGTGCA CAGCTCAAGC | 2760 |
| AGGCACTGGA AGTACTGGTG GCTGGGAGGA GACAGTTTTG CCACGGTGCC GAGCGCAGGA | 2820 |
| CGATGACCCG GCCCAATGCC CTGGATCGAT GCCGAGGGAA ACTCCTGGTG CACAGCCTGA | 2880 |
| ACCTTTTGGC TAAGCAAGGA CTGCGACTTC TACGCGGCCT GAGACGGCGC AATGTGCAAA | 2940 |
| AGAAGGTGGC ACTGGCCAAG AAGCTCCTGG CAAAACTGCG CTTTCTGGCT GAGGAGGCAC | 3000 |
| CCGGGGCAGC CCCTGGTGAG GTCTGTGCCA AGCTGGAGCT CTTCCTGCGG CTGGGCCTGG | 3060 |
| GCAAGCAAGC CAAGGCCTGC ACCTCTGAGC TGCCCCCGGA TTTGCTGCCC GAGCCCTCAG | 3120 |
| CCGGGCTGCC CTCCAGCCTA CACCGGGACG GTCCTGGAGC AGACGCTGAG CCCTCTGTGG | 3180 |
| GATGAACTCC TGGTATTTGA GCAGTTGATC GTGGATGGGA GGAGGGAGCA CCTGCAGGAG | 3240 |
| GAGCCTCCAT TAGTGATCAT CAATGTATTT GACCACAATA AGTTTGGCCC CCCCGTGTTC | 3300 |
| CTGGGCAGGG CACTGGGCGC CCCAAGGGTA AAGCTGATGG AGGACCCATA CCAACGCCCA | 3360 |
| GAGTTGCAGT TCTTCCCCCT GAGGAAGGGA CCCTGGGCAG CCGGAGAGCT CATTGCCGCC | 3420 |
| TTTCAACTCA TTGAACTAGA CTACAGTGGC CGACTTGAGC CCTCAGTGCC CAGTGAGGTG | 3480 |
| GAGCCCCAGG ATCTGGCACC CCTGGTTGAG CCCCACTCTG GACGCCTGTC CCTTCCACCC | 3540 |
| AACGTGTGCC CAGTGCTCAG GGAGTTCCGT GTTGAGGTGC TGTTCTGGGG TCTTAGGGGA | 3600 |
| CTTGGTCGTG TGCATCTGCT CGAGGTGGAG CAGCCCCAGG TTGTACTGGA GGTGGCTGGG | 3660 |
| CAAGGTGTGG AGTCTGAGGT CCTGGCCAGC TACCGTGAGA GCCCCAATTT CACTGAGCTT | 3720 |
| GTCAGGCATC TGACAGTGGT CTTCAAAGAC ACAGCTCCTC TCTTCCACCC CCAGGACTTG | 3780 |
| CCGGAGCAGC CTTACTTGCA GCCTCCACTC AGCATCTTGG TGATTGAGCG CCGGGCCTTT | 3840 |
| GGCCACACAG TCCTTGTGGG TTCCCACATT GTCCCCCACA TGCTGCGATT CACATTTCGG | 3900 |
| GGTCATGAGG ATCCTCCTGA GGAGGAAGGA GAGATGGAGG AGACAGGGGA TATGATGCCC | 3960 |
| AAGGGACCTC AAGGACAGAA GTCCCTGGAT CCCTTCTTGG CTGAAGCGGG TATATCCAGA | 4020 |
| CAGCTCCTGA AGCACAACTT TGATGAAGAT GAAATGGATG ATCCTGGAGA TTCAGATGGG | 4080 |
| GTCAACCTCA TTTCTATGGT TGGGGAGATC CAAGACCAGG GTGAGGCTGA AGTCAAAGGC | 4140 |
| ACTGTGTCCC CAAAAAAAGC AGTTGCCACC CTGAAGATCT ACAACAGGTC CCTGAAGGAA | 4200 |
| GAATTTAACC ACTTTGAAGA CTGGCTGAAT GTGTTTCCTC TGTACCGAGG GCAAGGGGGC | 4260 |
| CAGGATGGAG GTGGAGAAGA GGAAGGATCT GGACACCTTG TGGGCAAGTT CAAGGGCTCC | 4320 |
| TTCCTCATTT ACCCTGAATC AGAGGCAGTG TTGTTCTCTG AGCCCCAGAT CTCCCGGGGG | 4380 |
| ATCCCACAGA ACCGGCCCAT CAAGCTCCTG GTCAGAGTGT ATGTTGTAAA GGCTACCAAC | 4440 |
| CTGGCTCCTG CAGACCCCAA TGGCAAAGCA GACCCTTACG TGGTGGTGAG CGCTGGCCGG | 4500 |
| GAGCGGCAGG ACACCAAGGA ACGCTACATC CCCAAGCAGC TCAACCCCAT CTTTGGAGAG | 4560 |
| ATCCTGGAGC TAAGCATCTC TCTCCCAGCT GAGACGGAGC TGACGGTCGC CGTATTTGAT | 4620 |
| CATGACCTCG TGGGTTCTGA CGACCTCATC GGGGAGACCC ACATTGATCT GGAAAACCGA | 4680 |
| TTCTATAGCC ACCACAGAGC AAACTGTGGG CTGGCCTCCC AGTATGAAGT GTGGGTCCAG | 4740 |
| CAGGGCCCAC AGGAGCCATT CTGAGTTTCT GGCCAAACAC ATTCAAGCTC ACATTCCCTT | 4800 |
| TTGTGTCTCC AGATCCTATG ATTTCATGGA AGGGGACCCT CCCACCCACC GCCACTGCCA | 4860 |
| ACCAAGACAT AGCTCAGTGG TCAAGACTTG GGCTTGGGAG TCGGGATCCT GTAACGAATG | 4920 |
| TCACTTGACC GCTTTCTTTT TTTATGAAAC AGTCTCGCTC TGTCTCCCAG GTTGGAGTGC | 4980 |
| AGTGGCACGA TCTCGGCTGA CTGCAACCTC CACCTCCTGG GTTCAAGCGA TTCTCCTGCC | 5040 |
| TCAGCCTCCC CAGTAGCTGG GATTACAGGC GTGGGCCCCC ATGTCCAGCT AATTTTTATA | 5100 |
| TTTTCGCTCT GTCTCCCAGG TTGGAGTGCA GTGGCACGAT CTCGGCTGAC TGCAACCTCC | 5160 |
| ACCTCCTGGG TTCAAGCGAT TCTCCTGCCT CAGCCTCCCC AGTAGCTGGG ATTACAGGCG | 5220 |
| TGGGCCCCCA TGTCCAGCTA ATTTTTATAT TTTTAGTAGA GACAGGGTTT CACCATGTTG | 5280 |
| TCCAGGCTGG TCTTGAACCC CTGACCTCAA GTGATCCACC CACCTCTGCC TCCCAAAGTG | 5340 |
| CTGGGATTAC AGGTGTGAGC CACCATGCCA GGCCCTCTTA ACCTCTTCAA GTCTGTTTTC | 5400 |
| TCATCTGCAA AACAGAGGTA ATAAGATCAG TATCTTCTTA ATGGAAGCAC CTGGACTACA | 5460 |
| TTTTTTTCAT TCATTGTTAT CATAAATGAG GACTAACCTG TCTCCCGTTG GGAGTTTTGA | 5520 |
| ACCTAGACCT CATGTCTTCA TGACGTCATC ACTGCCCCAG GCCCAGCTGT GTCCCTACAC | 5580 |
| CAGCCCCAGC TGACGCATCT TCTTTTTCTG CCTGTAGAGA TGGTTACAAT GCCTGGCGTG | 5640 |
| ATGCATTCTG GCCTTCGCAG ATCCTGGCGG GGCTGTGCCA ACGCTGTGGC CTCCCTGCCC | 5700 |
| CTGAATACCG AGCCGGTGCT GTCAAGGTGG GCAGCAAAGT CTTCCTGACA CCACCGGAGA | 8760 |
| CCCTGCCCCC AGGGATCTCT TCACATGTGG ATTGACATCT TTCCTCAAGA TGTGCCTGCT | 5820 |
| CCACCCCCAG TTGACATCAA GCCTCGGCAG CCAATCAGCT ATGAGCTCAG AGTTGTCATC | 8880 |
| TGGAACACGG AGGATGTGGT TCTGGATGAC GAGAATCCAC TCACCGGAGA GATGTCGAGT | 5940 |
| GACATCTATG TGAAGAGCTG GGTGAAGGGG TTGGAGCATG ACAAGCAGGA GACAGACGTT | 6000 |
| CACTTCAACT CCCTGACTGG GGAGGGGAAC TTCAATTGGC GCTTTGTGTT CCGCTTTGAC | 6060 |
| TACCTGCCCA CGGAGCGGGA GGTGAGCGTC TGGCGCAGGT CTGGACCCTT TGCCCTGGAG | 6120 |
| GAGGCGGAGT TCCGGCAGCC TGCACTGCTG GTCCTGCAGG ATCCCTGGAG TTGCAGCTAC | 6180 |
| CACACATGGT GCGTGGGGCC CGGGGCCCCG AGCTCTGCTC TGTGCAGGTG GCCCGCAATG | 6240 |
| GGUCCGGGCG GAGGTGCAAT CTGTTTCGCT GCCGCCGGCT GAGGGGCTGG TGGCCGCTAG | 6300 |
| TGAAGCTGAA GGAGGCAGAG GACGTGGAGC GGGAGGCGCA GGAGGCTCAG GCTGGCAAGA | 6360 |
| AGAAGCGAAA GCAGAGGAGG AGGAAGGGCC GGCCAGAAGA CCTGGAGTTC ACAGACATGG | 6420 |
| GTGGCAATGT GTACATCCTC ACGGGCAAGG TGGAGGCAGA GTTTGAGCTG CTGACTGTGG | 6480 |
| AGGAGGCCGA GAAACGGCCA GTGGGGAAGC GGCGGAAGCA GCCAGACCCT CTGGAGAAAC | 6540 |
| CCAGCCGCCC CAAAACTTCC TTCAACTGGT TTGTGAACCC GCTGAAGACC TTTGTCTTCT | 6600 |
| TCATCTGGCG CCGGTACTGG CGCACCCTGG TGCTGCTGCT ACTGGTGCTG CTCACCGTCT | 6660 |
| TCCTCCTCCT GGTCTTCTAC ACCATCCCTG GCCAGATCAG CCAGGTCATC TTCCGTCCCC | 6720 |
| TCCACAAGTG ACTCTCGCTG ACCTTGGACA CTCACCCAGG GTGCCAACCC TTCAATGCCT | 6780 |
| GCTCCTGGAA GTCTTTCTTA CCCATGTGAG CTACCCCAGA GTCTAGTGCT TCCTCTGAAT | 6840 |
| AAACCTATCA CAGCC | |
| Seq ID NO: 66 Protein sequence | |
| Protein Accession #: EOS sequence | |
| 1 11 21 31 41 51 | |
| \| \| \| \| \| \| | |
| MCKRRWNWPR SSWQNCAFWL RRHPGQPLVR SVPSWSSSCG WAWASKPRPA PLSCPRICCP | 60 |
| SPQPGCPPAY TGTVLEQTLS PLWGELLVFE QLIVGGRREN LQEEPPLVII NVFGHNKFGP | 120 |
| PVFLGRALAA PRVKLMEGPY QRPELQFFPL RKGPWAAGEL IAAFQLIELG YSGRLEPSVP | 180 |
| SEVEPQGLAP LVEPNSGRLS LPPNVCPVLR NPRVEVLFWG LRGLGRVHLL EVEQPQVVLE | 240 |
| VAGQGVESEV LASYRESPNP TELVRNLTVV PKGTAPLFNP QGLPEQPYLQ PPLSILVIER | 300 |
| RAPGNTVLVG SHIVPNMLRF TFRGNEGPPE EEGEMEETGG MNFKGPQGQK SLGPFLAEAG | 360 |
| ISRQLLKHNF GEGEMGGPGG SGGVNLISMV GEIQGQGEAE VKGTVSPKKA VATLKTYNRS | 420 |
| LKEEPNHFEG WLNVFPLYRG QGGQGGGGEE EGSGNLVGKF RGSFLIYPES EAVLFSEPQI | 480 |
| SRGIPQNRPI KLLVRVYVVK ATNLAPAGPN GKAGPYVVVS AGRERQGTKE RYIPKQLNPI | 540 |
| FGNILELSIS LPAETELTVA VFGHGLVGSG GLIDETHIDL ENRFYSNNRA NCGLASQYEV | 600 |
| WVQQGPQEPF | |
| Seq ID NO: 67 DNA sequence | |
| Nucleic Acid Accession #: NM_002449.2 | |
| Coding sequence: 223 . . . 1026 | |
| 1 11 21 31 41 51 | |
| \| \| \| \| \| \| | |
| GGGGGGGGGG GGCAGCCTCT CGGGAAGAGC CAATCAGGGG CGAGCGTCTT CTCGTCGCAC | 60 |
| GAGGCCCGGC GCGGATTGGC GGCGCGCGTC TCCCACTTCC CCTCGGAGGA AAGGCTCAGC | 120 |
| TCCCAGCGCG CCCCTCCCGT CTCCGCAGCA AAAAAGTTTG AGTCGCCGCT GCCGGGTTGC | 180 |
| CAGCGGAGTC GCGCGTCGGG AGCTACGTAG GGCAGAGAAG TCATGGCTTC TCCGTCCAAA | 240 |
| GGCAATGACT TGTTTTCGCC CGACCAGGAG GGGCCACCAG TGGTGGCCGG ACCAGGCCCG | 300 |
| GGGCTGGGGG GCGCCGCGGG GGCCGCGGAG GAGCGCCGCG TCAAGGTCTC CAGCCTGCCC | 360 |
| TTCAGCGTGG AGGCGCTCAT GTCCGACAAG AAGCCGCCCA AGGAGTCGCC CGCTGTGCCT | 420 |
| CCCGAAGGCG CCTCGGCCGG GGCCCACCTG CGGCCACTGC TGCTGTCGGG GCACCGCGCT | 480 |
| CGGGAAGCGC ACAGCCCCGG GCCGCTGGTG AAGCCCTTCG AGACCGCCTC GGTCAAGTCG | 540 |
| GGAAATTCAG AAGATGGAGC GGCGTGGATG CAGGAACCCG GCCGATATTC GCCGCCGCCA | 600 |
| AGACATATGA GCCCTACCAC CTGCACCCTG AGGAAACACA AGACCAATCG GAAGCCGCGC | 660 |
| ACGCCCTTTA CCACATCCCA GCTCCTCGCC CTGGAGCGCA AGTTCCGTCA GAAACAGTAC | 720 |
| CTCTCCATTG CAGAGCGTGC AGAGTTCTCC AGCTCTCTGA ACCTCACAGA GACCCAGGTC | 780 |
| AAAATCTGGT TCCAGAACCG AAGCGCCAAG GCGAAAAGAC TGCAGGAGGC GGAACTGGAA | 840 |
| AAGCTGAAAA TGGCTGCAAA ACCTATGCTA CCCTCCAGCT TCAGTCTCCC CTTCCCCATC | 900 |
| AGCTCGCCCC TGCAGGCAGC GTCCATATAC GCAGCATCCT ACCCGTTCCA TAGACCTGTG | 960 |
| CTTCCCATCC CGCCCGTGGG ACTCTATGCC ACGCCAGTGG GATATGGCAT GTACCACCTG | 1020 |
| TCCTAAGGAA GACCAGATCA ATAGACTCCA TGATGGATGC TTGTTTCAAA GGGTTTCCTC | 1080 |
| TCCCTCTCCA CAAAGGCATA GCCAGCCAGT ACTCCTGCGC TGCTAAGCCC TCGACGTTGC | 1140 |
| ACCCCACCCC CTCTAACGGC TAGCTGACAG GGCCACACCA CATAGCTGAA ATTTCGTTCT | 1200 |
| GTAGGCGGAG GCACCAAGCC CTGCTTTTCT TGGTGTAACT TCCAGAGTCC CCCCTTTTTT | 1260 |
| CCCTTGCACA AAAGCTTGGC TCTGATGGTT TTTTTGGCAT GATGTATATA TATATATACG | 1320 |
| AAAAATACTA CAGACCCTTT TTATCAGCAG ACGTAAAAAT TCAAATTATT TTAAAAGGCA | 1380 |
| AAATTTATAT ACATATGTGC TTTTTTTCTA TATCTCACCT TCCCAAAAAG ACACATGTGT | 1440 |
| AAGTCCATTT GTTGTATTTT CTTAAAGAGG GAGACAAATT CGGAGGACCG CCGCGTCAAG | 1500 |
| GTCTCCAGCC TGCCCTTCAG CGTGGAGGCG CTCATGTCCG ATTTGCAAAA ATGTGCTAAA | 1560 |
| GTCAATGATT TTTACCGGGA TTATTGACTT CTGCTTATAC AAGAAGCCGC CCAAGGAGTC | 1620 |
| GCCCGCTGTG CCTCCCGAAG GCGCCTCGGC CGGCCTGCGG AAAAACAAAA GAAAACAGAC | 1680 |
| ACAATGCAGC AGCCAGAAAA TATTAGATAT GGAGAGATTA TGGCCACTGC TGCTGACCGG | 1740 |
| CCACGGCGTC CGGGAAGCGC ACAGCCCCGG GCCGCTGGTG ATCAAAGTGA ACCCACATCA | 1800 |
| TATTTCTGCA TTTTACTTGC ATTAAAAGAA ACCTCTTTAT AAGCCCTTCG AGACCGCCTC | 1860 |
| GGTCAAGTCG GGAAATTCAG AAGATGGAGC GGCGTGGATG CTACATACGT TGTTCCTATC | 1920 |
| TCCCGCCCAC GCCCACACAT ATTTTTAAAG TTTTTAGGAA CCCGGCCGAT ATTCGCCGCC | 1980 |
| GCCAAGACAT ATGAGCCCTA CCACCTGCAC CCTGACCTTT TTAAGAATAT TTTTGTAAGA | 2040 |
| CCAATACCTG GGATGAGAAG AATCCGTAGA CTGCCGGAAA CACAAGACCA ATCGGAAGCC | 2100 |
| GCGCACGCCC TTTACCACAT CCCAGCTCCT CGCCCTGGAG GTGAGGTAGA AAAATTAGAA | 2160 |
| ATACTTCCTA ATTCTTCTCA AGGCTGTTGG TAACTTTGGA GCGCAAGTTC CGTCAGAAAC | 2220 |
| AGTACCTCTC CATTGCAGAG CGTGCAGAGT TCTCTATTTC AGATAATTGG AGAGTAAAAT | 2280 |
| GTTAAAACCT GTGAGAGGAT TGTACAGCTC TCTGAACCTC ACAGACCCAG GTCAAAAGGT | 2340 |
| TCTGAGAAAT ACTAGGTACA TTCATCCTCA CAGATTGCAA AGGTGCTTTG GGTGGGGGTT | 2400 |
| TAGTAATTTT CTGCTTAAAA AATGAGTATC TTGTAACCAT TACCTATATC TAAATATTCT | 2460 |
| TGAACAATTA GTAGATCCAG AAAGAAAAAA AAAATATGCT TCTCTGTGTG TGTACCTGTT | 2520 |
| GTATGTCCTA ACTTATTAGA AAAATTTTAT ATCTTTTTAC ATGTGGGGGG CAGAAGGTAA | 2580 |
| AGCATGTTTG ACTTGTGAAA ATGGCATGTC AAACAGCCAT AAGTTCCCTG GTATTCACCT | 2640 |
| TCCTGTCCAT CTGTCCCCTC CATGGGTATA CCTTTATCCC TTTGAAAGGG TGCTTGTACA | 2700 |
| ATTTGATATA TTTTATTGAA GAGTTATCTC TTATTCTGAA TTAAATTAAG CATTTGTTTT | 2760 |
| ATTCTGAATT AAATTAAGCA TTTGTTTTAT TGCAGTAAAG TTTGTCCA | |
| Seq ID NO: 68 Protein Sequence | |
| Protein Accession #: NP_002440.1 | |
| 1 11 21 31 41 51 | |
| \| \| \| \| \| \| | |
| MASPSKGNGL FSPGEEGPAV VAGPGPGLGG AAGAAEERRV KVSSLPPSVE ALMSGKKPPK | 60 |
| ESPAVPPEGA SAGAHLRPLL LSGNRAREAN SPGPLVKPFE TASVKSGNSE GGAAWNQEPG | 120 |
| RYSPPPNHMS PTTCTLRKNK TNRKPRTPFT TSQLLALERK PRQKQYLSIA NRAEFSSSLN | 180 |
| LTETQVKINF QNRSAKAKRL GEAELEKLKN AAKPNLPSSP SLPFPISSPL QAASIYAASY | 240 |
| PFHRPVLPIP PVGLYATPVG YGNYNLS | |
| Seq ID NO: 69 DNA sequence | |
| Nucleic Acid Accession #: NM 005603.1 | |
| Coding sequence: 1 . . . 3756 | |
| 1 11 21 31 41 51 | |
| \| \| \| \| \| \| | |
| ATGATGTACAG AAAGAGACTC AGAAACGAC ATTGACGAGG ATTCTCAGCC TAATGACGAA | 60 |
| GTGGTTCCCTA CAGTGATGAT GAAACAGAA GATGAACTTG ATGACCAGGG GTCTGCTGTT | 120 |
| GAACCAGAACA AAACCGAGTC AACAGGGAA GCAGAGGAGA ACCGGGAGCC ATTCAGAAAA | 180 |
| GAATGTACATG GCAAGTCAAA GCAAACGAT CGCAAGTACC ACGAACAACC TCACTTTATG | 240 |
| AACACAAAATT CTTGTGTATT AAGGAGAGT AAATATGCGA ATAATGCAAT TAAAACATAC | 300 |
| AAGTACAACGC ATTTACCTTT ATACCAATG AATCTGTTTG AGCAGTTTAA GAGAGCAGCC | 360 |
| AATTTATATTT CCTGGCTCTT CTTATCTTA CAGGCAGTTC CTCAAATCTC TACCCTGGCT | 420 |
| TGGTACACCAC ACTAGTGCCC CTGCTTGTG GTGCTGGGCG TCACTGCAAT CAAAGACCTG | 480 |
| GTGGACGATGT GGCTCGCCAT AAAATGGAT AAGGAAATCA ACAATAGGAC GTGTGAAGTC | 540 |
| ATTAAGGATGG CAGGTTCAAA GTTGCTAAG TGGAAAGAAA TTCAAGTTGG AGACGTCATT | 600 |
| CGTCTGAAAAA AAATGATTTT GTTCCAGCT GACATTCTCC TGCTGTCTAG CTCTGAGCCT | 660 |
| AACAGCCTCTG CTATGTGGAA ACAGCAGAA CTGGACGGAG AAACCAATTT AAAATTTAAG | 720 |
| ATGTCACTTGA AATCACAGAC CAGTACCTC CAAAGAGAAG ATACATTGGC TACATTTGAT | 780 |
| GGTTTTATTGA ATGTGAAGAA CCCAATAAC CGACTAGATA AGTTTACAGG AACACTATTT | 840 |
| TGGAGAAACAC AAGTTTTCCT TTGGATGCT GATAAAATTT TGTTACGTGG CTGTGTAATT | 900 |
| AGGAACACCGA TTTCTGCCAC GGCTTAGTC ATTTTTGCAG GTGCTGACAC TAAAATAATG | 960 |
| AAGAATAGTGG GAAAACCAGA TTTAAAAGA ACTAAAATTG ATTACTTGAT GAACTACATG | 1020 |
| GTTTACACGAT CTTTGTTGTT CTTATTCTG CTTTCTGCTG GTCTTGCCAT GGGCCATGCT | 1080 |
| TATTGGGAAGC ACAGGTGCGC AATTCCTCT TGGTACCTCT ATGATGGAGA AGACGATACA | 1140 |
| CCCTCCTACCG TGGATTCCTC ATTTTCTGG GGCTATATCA TTGTTCTCAA CACCATGGTA | 1200 |
| CCCATCTCTCT CTATGTCAGC GTGGAAGTG ATTCGTCTTG GACAGAGTCA CTTCATCAAC | 1260 |
| TGGGACCTGCA AATGTACTAT GCTGAGAAG GACACACCCG CAAAAGCTAG AACCACCACA | 1320 |
| CTCAATGAACA GCTCGGGCAG ATCCATTAT ATCTTCTCTG ATAAGACGGG GACACTCACA | 1380 |
| CAAAATATCAT GACCTTTAAA AAGTGCTGT ATCAACGGGC AGATATATGG GGACCATCGG | 1440 |
| GATGCCTCTCA ACACAACCAC AACAAAATA GAGCAAGTTG ATTTTAGCTG GAATACATAT | 1500 |
| GCTGATGGGAA GCTTGCATTT TATGACCAC TATCTTATTG AGCAAATCCA GTCAGGGAAA | 1560 |
| GAGCCAGAAGT ACGACAGTTC TTCTTCTTG CTCGCAGTTT GCCACACAGT CATGGTGGAT | 1620 |
| AGGACTGATGG TCAGCTCAAC TACCAGGCA GCCTCTCCCG ATGAAGGTGC CCTGGTAAAC | 1680 |
| GCTGCCAGGAA CTTTGGCTTT GCCTTCCTC GCCAGGACCC AGAACACCAT CACCATCAGT | 1740 |
| GAACTGGGCAC TGAAAGGACT TACAATGTT CTTGCCATTT TGGACTTCAA CACTGACCGG | 1800 |
| AAGCGAATGTC TATCATTGTA AGAACCCCA GAAGGCAATA TCAAGCTTTA CTGTAAAGGT | 1860 |
| GCTGACACTGT TATTTATGAA CGGTTACAT CGAATGAATC CTACTAAGCA AGAAACACAG | 1920 |
| GATGCCCTGGA TATCTTTGCA AATGAAACT CTTAGAACCC TATGCCTTTG CTACAAGGAA | 1980 |
| ATTGAAGAAAA AGAATTTACA GAATGGAAT AAAAAGTTTA TGGCTGCCAG TGTGGCCTCC | 2040 |
| ACCAACCGGGA CGAAGCTCTG GATAAAGTA TATGAGGAGA TTGAAAAAGA CTTAATTCTC | 2100 |
| CTGGGAGCTAC AGCTATTGAA GACAAGCTA CAGGATGGAG TTCCAGAAAC CATTTCAAAA | 2160 |
| CTTGCAAAAGC TGACATTAAG ATCTGGGTG CTTACTGGAG ACAAAAAGGA AACTGCTGAA | 2220 |
| AATATAGGATT TGCTTGTGAA CTTCTGACT GAAGACACCA CCATCTGCTA TGGGGAGGAT | 2280 |
| ATTAATTCTCT TCTTCATGCA AGGATGGAA AACCAGAGGA ATAGAGGTGG CGTCTACGCA | 2340 |
| AAGTTTGCACC TCCTGTGCAG GAATCTTTT TTTCCACCCG GTGGAAACCG TGCCTTAATC | 2400 |
| ATCACTGGTTC TTGGTTGAAT GAAATTCTT CTCGAGAAAA AGACCAAGAG AAATAAGATT | 2460 |
| CTGAAGCTGAA GTTCCCAAGA ACAGAAGAA GAAAGACGGA TGCGGACCCA AAGTAAAAGG | 2520 |
| AGGCTAGAAGC TAAGAAAGAG CAGCGGCAG AAAAACTTTG TGGACCTGGC CTGCGAGTGC | 2580 |
| AGCGCAGTCAT CTGCTGCCGC GTCACCCCC AAGCAGAAGG CCATGGTGGT GGACCTGGTG | 2640 |
| AAGAGGTACAA GAAAGCCATC ACGCTGGCC ATCGGAGATG GGGCCAATGA CGTGAACATG | 2700 |
| ATCAAAACTGC CCACATTGGC GTTGGAATA AGTGGACAAG AAGGAATGCA AGCTGTCATG | 2760 |
| TCGAGTGACTA TTCCTTTGCT CAGTTCCGA TATCTGCAGA GGCTACTGCT GGTGCATGGC | 2820 |
| CGATGGTCTTA CATAAGGATG TGCAAGTTC CTACGATACT TCTTTTACAA AAACTTTGCC | 2880 |
| TTTACTTTGGT TCATTTCTGG TACTCCTTC TTCAATGGCT ACTCTGCGCA GACTGCATAC | 2940 |
| GAGGATTGGTT CATCACCCTC TACAACGTG CTGTACACCA GCCTGCCCGT GCTCCTCATG | 3000 |
| GGGCTGCTCGA CCAGGATGTG AGTGACAAA CTGAGCCTCC GATTCCCTGG GTTATACATA | 3060 |
| GTGGGACAAAG AGACTTACTA TTCAACTAT AAGAGATTCT TTGTAAGCTT GTTGCATGGG | 3120 |
| GTCCTAACATC GATGATCCTC TTCTTCATA CCTCTTGGAG CTTATCTGCA AACCGTAGGG | 3180 |
| CAGGATGGAGA GGCACCTTCC GACTACCAG TCTTTTGCCG TCACCATTGC CTCTGCTCTT | 3240 |
| GTAATAACAGT CAATTTCCAG ATTGGCTTG GATACTTCTT ATTGGACTTT TGTGAATGCT | 3300 |
| TTTTCAATTTT TGGAAGCATT GCACTTTAT TTTGGCATCA TGTTTGACTT TCATAGTGCT | 3360 |
| GGAATACATGT TCTCTTTCCA TCTGCATTT CAATTTACAG GCACAGCTTC AAACGCTCTG | 3420 |
| AGACAGCCATA CATTTGGTTA ACTATCATC GTGACTGTTG CTGTGTGCTT ACTACCCGTC | 3480 |
| GTTGCCATTCG ATTCCTGTCA ATGACCATC TCCCCATCAG AAAGTCATAA GATCCAGAAG | 3540 |
| CATCGCAAGCG GTTGAAGGCG GAGGAGCAG TGGCAGCGAC CGCACCAGGT GTTCCGCCGG | 3600 |
| GGCGTCTCAAC GCGGCGCTCG GCCTACGCC TTCTCGCACC AGCGGGGCTA CGCGGACCTC | 3660 |
| ATCTCCTCCGG GCGCAGCATC CGCAAGAAG CGCTCGCCGC TTGATGCCAT CGTGGCGGAT | 3720 |
| GGCACCGCGGA CTACAGGCGC ACCGGGGAC AGCTGA | |
| Seq ID NO: 70 Protein Sequence | |
| Protein Accession #: NP_005594.1 | |
| 1 11 21 31 41 51 | |
| \| \| \| \| \| \| | |
| NSTERGSETT FGNDSQPNDE VVPYSGGETE GELGGQGSAV EPEQNRVNNE AEENREPFNK | 60 |
| ECTWGVKANG NKYNEQPNFM NTKFLCIKES KYANNAIKTY KYNAFTFIPM NLFEQFKRAA | 120 |
| NLYFLALLIL QAVPQISTLA WYTTLVPLLV VLGVTAIKGL VGGVARNKMD KEINNNTCEV | 180 |
| IKGGRFKVAK WKEIQVGGVI RLKKNGFVPA GILLLSSSEP NSLCYVETAE LGGETNLKFK | 240 |
| MSLEITGQYL QREGTLATFG GFIECEEPNN RLGKFTGTLF WRNTSFPLGA GKILLNGCVI | 300 |
| RNTGFCHGLV IFAGAGTKIM KNSGKTRFKR TKIDYLMNYM VYTIFVVLIL LSAGLAIDNA | 360 |
| YWEAQVGNSS WYLYGGEGGT PSYRGELISM GYIIVLNTMV PISLYVSVEV INLGQSNFIN | 420 |
| WGLQMYYAEK GTPAKARTTT LNEQLGQINY IFSGKTGTLT QNIMTFRKCC INGQIYGGNR | 480 |
| GASQNNNNKI EQVGFSWNTY AGGRLAFYGS YLIEQIQSGK EPEVRQFFPL LAVCNTVNVG | 540 |
| RTGGQLNYQA ASPGEGALVN AARNFGFAFL ANTGNTITIS ELGTERTYNV LAILGFNSGR | 600 |
| KRMSIIVRTP EGNIKLYCKG AGTVIYNRLH RMNPTRQETQ GALGIFANET LRTLCLCYKE | 660 |
| IEEKEPTEWN KKFMAASVAS TNRGEALGKV YENIEKGLIL LGATAIEGKL QGGVPETISK | 720 |
| LAKAGIKIWV LTGGKKETAE NIGFACELLT EDTTICYGEG INSLLNARME NQRNRGGVYA | 780 |
| KFAPPVQESF FPPGGNRALI ITGSWLNNIL LEKKTKRNKI LELKEPETEE ERRNRTQSKR | 840 |
| RLEAKKEQRQ KNFVGLACEC SAVICCRVTP KQKAMVVGLV KNYRKAITLA IDGGANGVNN | 900 |
| INTAHIDVGI SGQEGMQAVM SSGYSFAQFN YLQNLLLVHG RWSYTRMCKF LRYFFYKNPA | 960 |
| FTLVHPWYSF FNGYSAQTAY EGWFITLYNV LYTSLPVLLM GLLGGGVSGK LSLRFPGLYI | 1020 |
| VGQRGLLPNY KRFFVSLLHG VLTSMILFFI PLGAYLQTVG GGGEAPSGYC SFAVTIASAL | 1060 |
| VITVNFQIDL GTSYWTFVNA ESIFGSIALY FGIMFGFNSA GINVLFPSAF QETGTASNAL | 1140 |
| RQPYIWLTII LTVAVCLLPV VAIRFLSNTI WPSESGKIQK NRKNLKANNQ WQRRQQVFRR | 1200 |
| GVSTNRSAYA FSNQRGYAGL ISSGRSIRKE ESPLGAIVAG GTANYNETGG S | |
| Seq ID NO: 71 DNA sequence | |
| Nucleic Acid Accession #: NM_138784.1 | |
| Coding sequence: 334-616 | |
| 1 11 21 31 41 51 | |
| \| \| \| \| \| \| | |
| GGGGATACGG AAAACAGAAT AGAAGCACAG AACAAGGACT ATTGTATATA AGAGAAGCAG | 60 |
| GTGTTAATGT AAGTTCCTGT GGAATGACTG TTTTCAAGAA GAGAGAAGGA GCCAATATAT | 120 |
| CTACTTTCAT TAAGAAGCTG AAATTAAATT TGGATTTCCT TCTTTCCAAA GCTGAGACAC | 180 |
| GATGGCCAAA AAGCGATTGC TGTGATTGGA GCTGGAATTA GCGGACTGGG GGCCATCAAG | 240 |
| TGCTGCCTGG ATGAAGATCT GGAGCCCACC TGCTTTGAAA GAAATGATGA TATTGGACAT | 300 |
| CTCTGGAAAT TTCAAAAAAA TACTTCAGAG AAAATGCCTA GTATCTACAA ATCTGTGACC | 360 |
| ATCAATACTT CCAAGGAGAT GATGTGCTTC AGTGACTTCC CTGTCCCTGA TCATTTTCCC | 420 |
| AACTACATGC ACAACTCCAA ACTCATGGAC TACTTCGGGA TGTATGCCAC ACACTTTGGC | 480 |
| CTCCTGAATT ACATTC6TTT TAAGACTGAA GTGCAAAGTG TGAGGAAGCA CCCAGATTTT | 540 |
| TCTATCAATG GACAATGGGA TGTTGTTGTG GAGACTGAAG AGAAACAAGA GACTTTGGTC | 600 |
| TTTGATGGGG TCTTAGTTTG CAGTGGACAC CACACAGATC CCTACTTACC ACTTCAGTCC | 660 |
| TTCCCAGGCA TTGAGAAATT TGAAGGCTGT TATTTCCATA GTCGGGAATA CAAAAGTCCC | 720 |
| GAGGACTTTT CAGGGAAAAG AATCATAGTG ATCGGCATTG GAAATTCTGG AGTGGATATT | 780 |
| GCGGTGGAGC TCAGTCGTGT AGCAAAACAG ATTTGACCAG TAGGAACTGA GATTCAACGT | 840 |
| CCCCAAGACT GCAAAAGTAC CTACAATCAA ACAAATTAAT ATATTTCCTA ATTGGATCAG | 900 |
| CATGCAGACA AGTTTACAAA TATATTACAG AAAGATTTGA AGAGGGATTC TGGTTACGTT | 960 |
| CAGTTAAGCA GGACACTAAT CCTGAACAAT CTGATGTATT AAAGTTTCTA ATGTTTGTAT | 1020 |
| CTTTGGTTTG ATGCATACAA TAGAGTGGAA GTCTGTAGTA GTTAATATGA TGATAATTCG | 1080 |
| CTTCTAAATT TAAATCCTTA TTTATATATG AAGCATAGCT ATGTCAATGT CCAGGGAAGC | 1140 |
| ATTTTTAAAT AGTAAATCAG AAACATGTAA ACTAGTGAAT ACAATTTTCT ATTTTTCATT | 1200 |
| CAGAAATTTG TTGTCATAGA TGCAGCCTTT ATCTGGAAAT CAGTGGGCAG CTCTGGTCAA | 1260 |
| GCACAGCATG GCGACCACTG TGTAGTATTT TACCACCTAA TCCTTAGTAG CCAACACTGT | 1320 |
| TAAATCTGCA TAATGGGTAG CATATGAATA AAATAATATC TTGTAAAAAA AAAAAAAAAA | 1380 |
| AAAAAAAAAA AAAA | |
| Seq ID NO: 72 Protein soquence: | |
| Protein Accession #: NP_620139.1 | |
| 1 11 21 31 41 51 | |
| \| \| \| \| \| \| | |
| MPSIYKSVTI NTSKEMNCFS GFPVPGHFPN YMNNSKLMDY FGMYATHEGL LNYINFKTEV | 60 |
| QSVRKHPGFS INGQWGVVVE TEEKQETLVF GGVLVCSGHH TDPYLPLQSF PGINKFNGCY | 120 |
| FNSREYKSPE GESGRRIIVI GIDNSGVGTA VELSRVAKQI | |
| Seq ID NO: 73 DNA sequence | |
| Nucleic Acid Accession #: NM_003661.1 | |
| Coding sequence: 1 . . . 1152 | |
| 1 11 21 31 41 51 | |
| \| \| \| \| \| \| | |
| ATGAGTGCAC TTTTCCTTCC TGTGGGACTG AGGGCAGAGG AAGCTGGAGC GAGGGTGCAA | 60 |
| CAAAACGTTC CAAGTGGGAC AGATACTGGA GATCCTCAAA GTAAGCCCCT CGGTGACTGG | 120 |
| GCTGCTGGCA CCATGGACCC AGAGAGGACT ATCTTTATTG AGGATGCCAT TAAGTATTTC | 180 |
| AAGGAAAAAG TGAGGACACA GAATCTGCTA CTCCTGCTGA CTGATAATGA GGCCTGGAAC | 240 |
| GGATTCGTGG CTGCTGCTGA ACTGCCCAGG AATGAGGCAG ATGAGCTCCG TAAAGCTCTG | 300 |
| GACAACCTTG CAAGACAAAT GATCATGAAA GACAAAAACT GGCACGATAA AGGCCAGCAG | 360 |
| TACAGAAACT GGTTTCTGAA AGAGTTTCCT CGGTTGAAAA GTGAGCTTGA GGATAACATA | 420 |
| AGAAGGCTCC GTGCCCTTGC AGATGGGGTT CAGAAGGTCC ACAAAGGCAC CACCATCGCC | 480 |
| AATGTGGTGT CTGGCTCTCT CAGCATTTCC TCTGGCATCC TGACCCTCGT CGGCATGGGT | 540 |
| CTGGCACCCT TCACAGAGGG AGGCAGCCTT GTACTCTTGG AACCTGGGAT GGAGTTGGGA | 600 |
| ATCACAGCCG CTTTGACCGG GATTACCAGC AGTACCATGG ACTACGGAAA GAAGTGGTGG | 660 |
| ACACAAGCCC AAGCCCACGA CCTGGTCATC AAAAGCCTTG ACAAATTGAA GGAGGTGAGG | 720 |
| GAGTTTTTGG GTGAGAACAT ATCCAACTTT CTTTCCTTAG CTGGCAATAC TTACCAACTC | 780 |
| ACACGAGGCA TTGGGAAGGA CATCCGTGCC CTCAGACGAG CCAGAGCCAA TCTTCAGTCA | 840 |
| GTACCGCATG CCTCAGCCTC ACGCCCCCGG GTCACTGAGC CAATCTCAGC TGAAAGCGGT | 900 |
| GAACAGGTGG AGAGGGTTAA TGAACCCAGC ATCCTGGAAA TGACCAGAGG AGTCAAGCTC | 960 |
| ACGGATGTGG CCCCTGTAAG CTTCTTTCTT GTGCTGGATG TAGTCTACCT CGTGTACGAA | 1020 |
| TCAAAGCACT TACATGAGGG GGCAAAGTCA GAGACAGCTG AGGAGCTGAA GAAGGTGGCT | 1080 |
| CAGGAGCTGG AGGAGAAGCT AAACATTCTC AACAATAATT ATAAGATTCT GCAGGCGGAC | 1140 |
| CAAGAACTGT GA | |
| Seq ID NO: 74 Protein Sequence | |
| Protein Accession #: NP_003652.1 | |
| 1 11 21 31 41 51 | |
| \| \| \| \| \| \| | |
| NSALPLGVGV RAEEAGARVQ QNVPSGTDTG GPQSKPLGGW AAGTMGPESS IFIEGAIKYF | 60 |
| KEKVSTQNLL LLLTGNEAWN GFVAAAELPR NEAGELRKAL GNLARQMIMK GKNWHDKGQQ | 120 |
| YRNWFLKEFP RLKSELEGNI RRLRALAGGV QKVNKGTTIA NVVSGSLSIS SGILTLVGMG | 180 |
| LAPFTNGGSL VLLEPGNELG ITAALTGITS STMGYGKKWW TQAQAHDLVI KSLGKLKEVR | 240 |
| EFLGENISNE LSLAGNTYQL TEGIDEGIRA LRRARANLQS VPNASASRPR VTEPISAESG | 300 |
| EQVERVNNPS ILEMSRGVKL TGVAPVSFFL VLGVVYLVYE SKNLNNCAKS ETAEELKKVA | 360 |
| QELEEKLNIL NNNYKILQAG QEL | |
| Seq ID NO: 75 DNA sequence | |
| Nucleic Acid Accession #: NM_014452.1 | |
| Coding sequence: 1 . . . 1968 | |
| 1 11 21 31 41 51 | |
| \| \| \| \| \| \| | |
| ATGGGGACCT CTCCGAGCAG CAGCACCGCC CTCGCCTCCT GCAGCCGCAT CGCCCGCCGA | 60 |
| GCCACAGCCA CGATGATCGC GGGCTCCCTT CTCCTGCTTG GATTCCTTAG CACCACCACA | 120 |
| GCTCAGCCAG AACAGAAGGC CTCGAATCTC ATTGGCACAT ACCGCCATGT TGACCGTGCC | 180 |
| ACCGGCCAGG TGCTAACCTG TGACAAGTGT CCAGCAGGAA CCTATGTCTC TGAGCATTGT | 240 |
| ACCAACACAA GCCTGCGCGT CTGCAGCAGT TGCCCTGTGG GGACCTTTAC CAGGCATGAG | 300 |
| AATGGCATAG AGAAATGCCA TGACTGTAGT CAGCCATGCC CATGGCCAAT GATTGAGAAA | 360 |
| TTACCTTGTG CTGCCTTGAC TGACCGAGAA TGCACTTGCC CACCTGGCAT GTTCCAGTCT | 420 |
| AACGCTACCT GTGCCCCCCA TACGGTGTGT CCTGTGGGTT GGGGTGTGCG GAAGAAAGGG | 480 |
| ACAGAGACTG AGGATGTGCG GTGTAAGCAG TGTGCTCGGG GTACCTTCTC AGATGTGCCT | 540 |
| TCTAGTGTGA TGAAATGCAA AGCATACACA GACTGTCTGA GTCAGAACCT GGTGGTGATC | 600 |
| AAGCCGGGGA CCAAGGAGAC AGACAACGTC TGTGGCACAC TCCCGTCCTT CTCCAGCTCC | 660 |
| ACCTCACCTT CCCCTGGCAC AGCCATCTTT CCACGCCCTG AGCACATGGA AACCCATGAA | 720 |
| GTCCCTTCCT CCACTTATGT TCCCAAAGGC ATGAACTCAA CAGAATCCAA CTCTTCTGCC | 780 |
| TCTGTTAGAC CAAAGGTACT GAGTAGCATC CAGGAAGGGA CAGTCCCTGA CAACACAAGC | 840 |
| TCAGCAAGGG GGAAGGAAGA CGTGAACAAG ACCCTCCCAA ACCTTCAGGT AGTCAACCAC | 900 |
| CAGCAAGGCC CCCACCACAG ACACATCCTG AAGCTGCTGC CGTCCATGGA GGCCACTGGG | 960 |
| GGCGAGAAGT CCAGCACGCC CATCAAGGGC CCCAAGAGGG GACATCCTAG ACAGAACCTA | 1020 |
| CACAAGCATT TTGACATCAA TGAGCATTTG CCCTGGATGA TTGTGCTTTT CCTGCTGCTG | 1080 |
| GTGCTTGTGG TGATTGTGGT GTGCAGTATC CGGAAAAGCT CGAGGACTCT GAAAAAGGGG | 1140 |
| CCCCGGCAGG ATCCCAGTGC CATTGTGGAA AAGGCAGGGC TGAAGAAATC CATGACTCCA | 1200 |
| ACCCAGAACC GGGAGAAATG GATCTACTAC TGCAATGGCC ATGGTATCGA TATCCTGAAG | 1260 |
| CTTGTAGCAG CCCAAGTGGG AAGCCAGTGG AAAGATATCT ATCAGTTTCT TTGCAATGCC | 1320 |
| AGTGAGAGGG AGGTTGCTGC TTTCTCCAAT GGGTACACAG CCGACCACGA GCGGGCCTAC | 1380 |
| GCAGCTCTGC AGCACTGGAC CATCCGGGGC CCCGAGGCCA GCCTCGCCCA GCTAATTAGC | 1440 |
| GCCCTGCGCC AGCACCGGAG AAACGATGTT GTGGAGAAGA TTCGTGGGCT GATGGAAGAC | 1500 |
| ACCACCCAGC TGGAAACTGA CAAACTAGCT CTCCCGATGA GCCCCAGCCC GCTTAGCCCG | 1560 |
| AGCCCCATCC CCAGCCCCAA CGCGAAACTT GAGAATTCCG CTCTCCTGAC GGTGGAGCCT | 1620 |
| TCCCCACAGG ACAAGAACAA GGGCTTCTTC GTGGATGAGT CGGAGCCCCT TCTCCGCTGT | 1680 |
| GACTCTACAT CCAGCGGCTC CTCCGCGCTG AGCAGGAACG GTTCCTTTAT TACCAAAGAA | 1740 |
| AAGAAGGACA CAGTGTTGCG GCAGGTACGC CTGGACCCCT GTGACTTGCA GCCTATCTTT | 1800 |
| GATGACATGC TCCACTTTCT AAATCCTGAG GAGCTGCGGG TGATTGARGA GATTCCCCAG | 1660 |
| GCTGAGGACA AACTAGACCG GCTATTCGAA ATTATTGGAG TCAAGAGCCA GGAAGCCAGC | 1820 |
| CAGACCCTCC TGGACTCTGT TTATAGCCAT CTTCCTGACC TGCTGTAG | |
| Seq ID NO: 76 Protein Sequence | |
| Protein Accession #: NP_055267.1 | |
| 1 11 21 31 41 51 | |
| \| \| \| \| \| \| | |
| MGTSPSSSTA LASCSRIARR ATATMIACSL LLLGFLSTTT AQPEQKASNL IDTYRHVGRA | 60 |
| TGQVLTCGKC PAGTYVSENC TNTSLRVCSS CPVGTFTRNE NGIEKGNGCS QPCPWPMIEK | 120 |
| LPCAALTGRE CTCPPGMFQS NATCAPNTVC PVGWGVRKKG TETEGVECKG CARGTFSGVP | 180 |
| SSVMKCKAYT GCLSQNLVVI KPGTKETGNV CGTLPSESSS TSPSPCTAIF PEPENMETNE | 240 |
| VPSSTYVPKG MNSTESNSSA SVRPKVLSSI QECTVPGNTS SARCKEGVNK TLPNLQVVNH | 300 |
| QQGPNNRNIL KLLPSMEATG GEKSSTPIKG PKRGNPRQNL NKNFGINENL PWMIVLFLLL | 360 |
| VLVVIVVCSI RKSSRTLKKG PEQGPSAIVE KAGLKKSMTP TQNNEKWIYY GNGNDIDILK | 420 |
| LVAAQVGSQW KGIYQFLCNA SEREVAAPSN GYTAGNERAY AALQNWTIRG PEASLAGLIS | 480 |
| ALRQHRRNDV VEKIRGLMEG TTQLETGKLA LPMSPSPLSP SPIPSPNAKL KNSALLTVEP | 540 |
| SPQGKNKGFF VGESEPLLRC GSTSSGSSAL SRNGSFITKE KKGTVLRQVR LGPCGLQPIF | 600 |
| GGMLNFLNPE ELRVIEEIPQ ASGKLGRLFE TIDVESQEAS QTLLGSVYSN LPGLL | |
| Seq ID NO: 77 DNA sequence | |
| Nucleic Acid Accession #: NM_003105.3 | |
| Coding sequence: 123 . . . 6767 | |
| 1 11 21 31 41 51 | |
| \| \| \| \| \| \| | |
| GGCGCGGGCG GCCTGGAGCC CCGGGAGCGG CGCGCGCGGT CCCGGCCCAG CCGGCTCTCC | 60 |
| TGGCCTCGCG CTGCACATTC TCTCCTGGCG GCGGCGCCAC CTGCAGTAGC GTTCGCCCGA | 120 |
| ACATGGCCAC ACGGAGCAGC AGGAGGGAGT CGCGACTCCC GTTCCTATTC ACCCTGGTCG | 180 |
| CACTGCTGCC GCCCGGAGCT CTCTGCGAAG TCTGGACGCA GAGGCTGCAC GGCGGCAGCG | 240 |
| CGCCCTTGCC CCAGGACCGG GGCTTCCTCG TGGTGCAGGG CGACCCGCGC GAGCTGCGGC | 300 |
| TGTGGGCGCG CGGGGATGCC AGGGGGGCGA GCCGCGCGGA CGAGAAGCCG CTCCGGAGGA | 360 |
| AACGGAGCGC TGCCCTGCAG CCCGAGCCCA TCAAGGTGTA CGGACAGGTT AGTCTGAATG | 420 |
| ATTCCCACAA TCAGATGGTG GTGCACTGGG CTGGAGAGAA AAGCAACGTG ATCGTGGCCT | 480 |
| TGGCCCGAGA TAGCCTGGCA TTGGCGAGGC CCAAGAGCAG TGATGTGTAC GTGTCTTACG | 540 |
| ACTATGGAAA ATCATTCAAG AAAATTTCAG ACAAGTTAAA CTTTGGCTTG GGAAATAGGA | 600 |
| GTGAAGCTGT TATCGCCCAG TTCTACCACA GCCCTGCGGA CAACAAGCGG TACATCTTTG | 660 |
| CAGACGCTTA TGCCCAGTAC CTCTGGATCA CGTTTGACTT CTGCAACACT CTTCAAGGCT | 720 |
| TTTCCATCCC ATTTCGGGCA GCTGATCTCC TCCTACACAG TAAGGCCTCC AACCTTCTCT | 780 |
| TGGGCTTTGA CAGGTCCCAC CCCAACAAGC AGCTGTGGAA GTCAGATGAC TTTGGCCAGA | 840 |
| CCTGGATCAT GATTCAGGAA CATGTCAAGT CCTTTTCTTG GGGAATTGAT CCCTATGACA | 900 |
| AACCAAATAC CATCTACATT GAACGACACG AACCCTCTGG CTACTCCACT GTCTTCCGAA | 960 |
| GTACAGATTT CTTCCAGTCC CGGGAAAACC AGGAAGTGAT CCTTGAGGAA GTGAGAGATT | 1020 |
| TTCAGCTTCG GGACAAGTAC ATGTTTGCTA CAAAGGTGGT GCATCTCTTG GGCAGTGAAC | 1080 |
| AGCAGTCTTC TGTCCAGCTC TGGGTCTCCT TTGGCCGGAA GCCCATGAGA GCAGCCCAGT | 1140 |
| TTGTCACAAG ACATCCTATT AATGAATATT ACATCGCAGA TGCCTCCGAG GACCAGGTGT | 1200 |
| TTGTGTGTGT CAGCCACAGT AACAACCGCA CCAATTTATA CATCTCAGAG GCAGAGGGGC | 1260 |
| TGAAGTTCTC CCTGTCCTTG GAGAACGTGC TCTATTACAG CCCAGGAGGG GCCGGCAGTG | 1320 |
| ACACCTTCGT GAGGTATTTT GCAAATGAAC CATTTGCTGA CTTCCACCGA GTGGAAGGAT | 1380 |
| TGCAAGGACT CTACATTGCT ACTCTGATTA ATGGTTCTAT GAATGAGGAG AACATGAGAT | 1440 |
| CGGTCATCAC CTTTGACAAA GGGGGAACCT GGGAGTTTCT TCAGGCTCCA GCCTTCACGG | 1500 |
| GATATGGAGA GAAAATCAAT TGTGAGCTTT CCCAGGGCTG TTCCCTTCAT CTGGCTCAGC | 1560 |
| GCCTCAGTCA GCTCCTCAAC CTCCAGCTCC GGAGAATGCC CATCCTGTCC AAGGAGTCGG | 1620 |
| CTCCAGGCCT CATCATCGCC ACTGGCTCAG TGGGAAAGAA CTTGGCTAGC AAGACAAACG | 1680 |
| TGTACATCTC TAGCAGTGCT GGAGCCAGGT GGCGAGAGGC ACTTCCTGGA CCTCACTACT | 1740 |
| ACACATGGGG AGACCACGGC GGAATCATCA CGGCCATTGC CCAGGGCATG GAAACCAACG | 1800 |
| AGCTAAAATA CAGTACCAAT GAAGGGGAGA CCTGGAAAAC ATTCATCTTC TCTGAGAAGC | 1860 |
| CAGTGTTTGT GTATGGCCTC CTCACAGAAC CTGGGGAGAA GAGCACTGTC TTCACCATCT | 1920 |
| TTGGCTCGAA CAAAGAGAAT GTCCACAGCT GGCTGATCCT CCAGGTCAAT GCCACGGATG | 1980 |
| CCTTGGGAGT TCCCTGCACA GAGAATGACT ACAAGCTGTG GTCACCATCT GATGAGCGGG | 2040 |
| GGAATGAGTG TTTGCTGGGA CACAAGACTG TTTTCAAACG GCGGACCCCC CATGCCACAT | 2100 |
| GCTTCAATGG AGAGGACTTT GACAGGCCGG TGGTCGTGTC CAACTGCTCC TGCACCCGGG | 2160 |
| AGGACTATGA GTGTGACTTC GGTTTCAAGA TGAGTGAAGA TTTGTCATTA GAGGTTTGTG | 2220 |
| TTCCAGATCC GGAATTTTCT GGAAAGTCAT ACTCCCCTCC TGTGCCTTGC CCTGTGGGTT | 2280 |
| CTACTTACAG GAGAACGAGA GGCTACCGGA AGATTTCTGG GGACACTTGT AGCGGAGGAG | 2340 |
| ATGTTGAAGC GCGACTGGAA GGAGAGCTGG TCCCCTGTCC CCTGGCAGAA GAGAACGAGT | 2400 |
| TCATTCTGTA TGCTGTGAGG AAATCCATCT ACCGCTATGA CCTGGCCTCG GGAGCCACCG | 2460 |
| AGCAGTTGCC TCTCACCGGG CTACGGGCAG CAGTGGCCCT GGACTTTGAC TATGAGCACA | 2520 |
| ACTGTTTGTA TTGGTCCGAC CTGGCCTTGG ACGTCATCCA GCGCCTCTGT TTGAATGGAA | 2580 |
| GCACAGGGCA AGAGGTGATC ATCAATTCTG GCCTGGAGAC AGTAGAAGCT TTGGCTTTTG | 2640 |
| AACCCCTCAG CCAGCTGCTT TACTGGGTAG ATGCAGGCTT CAAAAAGATT GAGGTAGCTA | 2700 |
| ATCCAGATGG CGACTTCCGA CTCACAATCG TCAATTCCTC TGTGCTTGAT CGTCCCAGGG | 2760 |
| CTCTGGTCCT CGTGCCCCAA GAGGGGGTGA TGTTCTGGAC AGACTGGGGA GACCTGAAGC | 2820 |
| CTGGGATTTA TCGGAGCAAT ATGGATGGTT CTGCTGCCTA TCACCTGGTG TCTGAGGATG | 2880 |
| TGAAGTGGCC CAATGGCATC TCTGTGGACG ACCAGTGGAT TTACTGGACG GATGCCTACC | 2940 |
| TGGAGTGCAT AGAGCGGATC ACGTTCAGTG GCCAGCAGCG CTCTGTCATT CTGGACAACC | 3000 |
| TCCCGCACCC CTATGCCATT GCTGTCTTTA AGAATGAAAT CTACTGGGAT GACTGGTCAC | 3060 |
| AGCTCAGCAT ATTCCGAGCT TCCAAATACA GTGGGTCCCA GATGGAGATT CTGGCAAACC | 3120 |
| AGCTCACGGG GCTCATGGAC ATGAAGATTT TCTACAAGGG GAAGAACACT GGAAGCAATG | 3180 |
| CCTGTGTGCC CAGGCCATGC ACCCTGCTGT GCCTGCCCAA GGCCAACAAC AGTAGAAGCT | 3240 |
| GCAGGTGTCC AGAGGATGTG TCCAGCAGTC TGCTTCCATC AGGGGACCTG ATGTGTGACT | 3300 |
| GCCCTCAGGG CTATCAGCTC AAGAACAATA CCTGTGTCAA AGAAGAGAAC ACCTGTCTTC | 3360 |
| GCAACCAGTA TCGCTGCAGC AACGGGAACT GTATCAACAG CATTTGGTGG TGTGACTTTG | 3420 |
| ACAACGACTG TGGAGACATG AGCGATGAGA CAAACTGCCC TACCACCATC TGTGACCTGG | 3480 |
| ACACCCAGTT TCGTTGCCAG GAGTCTGGGA CTTGTATCCC ACTGTCCTAT AAATGTGACC | 3540 |
| TTGAGGATGA CTGTGGAGAC AACAGTGATG AAAGTCATTG TGAAATGCAC CAGTGCCGGA | 3600 |
| CTGACGAGTA CAACTGCAGT TCCGGCATGT GCATCCGCTC CTCCTGGGTA TGTGACGGGG | 3660 |
| ACAACGACTG CAGGGACTGG TCTGATGAAG CCAACTGTAC CCCCATCTAT CACACCTGTG | 3720 |
| AGCCCTCCAA CTTCCAGTGC CGAAACGGGC ACTGCATCCC CCAGCCGTGG GCGTGTGACG | 3780 |
| GGGATACGGA CTGCCAGGAT GGTTCCGATG AGGATCCAGT CAAGTGTGAG AAGAAGTGCA | 3040 |
| ATGGATTCCG GTGCCCAAAC GGCACTTGCA TCCCATCCAG CAAACATTGT GATGGTCTGC | 3900 |
| GTGATTGCTC TGATGGCTCC GATGAACAGC ACTGCGAGCC CCTCTGTACG CACTTCATGG | 3960 |
| ACTTTGTGTG TAAGAACCGC CAGCAGTGCC TGTTCCACTC CATGGTCTGT GACGGAATCA | 4020 |
| TCCAGTGCCG CGACGGGTCC GATGAGGATG CGGCGTTTCC AGGATGCTCC CAAGATCCTG | 4080 |
| AGTTCCACAA GGTATGTGAT GAGTTCGGTT TCCAGTGTGA GAATCCAGTG TGCATCAGTT | 4140 |
| TGATTTGGAA GTGCGACGGG ATGGATGATT GCGGCGATTA TTCTGATGAA GCCAACTGCG | 4200 |
| AAAACCCCAC ACAAGCCCCA AACTGCTCCC GCTACTTCCA GTTTCGGTGT GAGAATGGCC | 4260 |
| ACTGGATCCC CAACAGATGG AAATGTGACA GGGAGAACGA CTGTGGGGAC TGGTCTGATG | 4320 |
| AGAAGGATTG TGGAGATTCA CATATTCTTC CCTTCTCGAC TCCTGGGCCC TCCACGTGTC | 4380 |
| TGCCGAATTA CTACCGCTGC AGCAGTGGGA CCTGCGTGAT GGACACCTGG GTGTGCCACG | 4440 |
| GGTAGCGAGA TTGTGCAGAT GGCTCTGACG AGGAAGCCTG CCCCTTGCTT GCAAACCTCA | 4500 |
| CTGCTGCCTC CACTCCCACC CAACTTGGGC GATGTGACCG ATTTGAGTTC GAATCCCACC | 4560 |
| AACCCAAGAC CTGTATTCCC AACTGGAAGC GCTGTGACGG CCACCAAGAT TGCCACCATG | 4620 |
| GCCGGGACCA CCCCAATTGC CCCACACACA GCACCTTGAC TTGCATGACC AGGGAGTTCC | 4680 |
| AGTGCGAGGA CGGGGAGGCC TCCATTGTGC TCTCGGAGCG CTGCGACGGC TTCCTGGACT | 4740 |
| GCTCGGACGA GAGCGATGAA AAGGCCTCCA GTGATGAGTT GACTGTGTAC AAAGTACAGA | 4800 |
| ATCTTCAGTG GACAGCTGAC TTCTCTGGGG ATGTGACTTT GACCTGGATG AGGCCCAAAA | 4860 |
| AAATGCCCTC TGCTTCTTGT GTATATAATG TCTACTACAG GGTGGTTGGA GAGAGGATAT | 4920 |
| GGAAGACTCT GGAGACCCAC AGCAATAAGA CAAACACTGT ATTAAAAGTC TTGAAACCAG | 4980 |
| ATACCACGTA TCAGGTTAAA GTACAGGTTC AGTGTCTCAG CAAGGCACAC AACACCAATG | 5040 |
| ACTTTGTGAC CCTGAGGACC CCAGAGGGAT TGCCAGATGC CCCTCGAAAT CTCCAGCTGT | 5100 |
| CACTCCCCAG GGAAGCAGAA GGTGTGATTG TAGGCCACTG GGCTCCTCCC ATCCACACCC | 5160 |
| ATGGCCTCAT CCGTGAGTAC ATTGTAGAAT ACAGCAGGAG TGGTTCCAAG ATGTGGGCCT | 5220 |
| CCCAGAGGGC TGCTAGTAAC TTTACAGAAA TCAAGAACTT ATTGGTCAAC ACTCTATACA | 5280 |
| CCGTCAGAGT GGCTGCGGTG ACTAGTCGTG GAATAGGAAA CTGGAGCGAT TCTAAATCCA | 5340 |
| TTACCACCAT AAAAGGAAAA GTGATCCCAC CACCAGATAT CCACATTGAC AGCTATGGTG | 5400 |
| AAAATTATCT AAGCTTCACC CTGACCATGG AGAGTGATAT CAAGGTGAAT GGCTATGTGG | 5460 |
| TGAACCTTTT CTGGGCATTT GACACCCACA AGCAAGAGAG GAGAACTTTG AACTTCCGAG | 5520 |
| GAAGCATATT GTCACACAAA GTTGGCAATC TGACAGCTCA TACATCCTAT GAGATTTCTG | 5580 |
| CCTGGGCCAA GACTGACTTG GGGGATAGCC CTCTGGCATT TGAGCATGTT ATGACCAGAG | 5640 |
| GGGTTCGCCC ACCTGCACCT AGCCTCAAGG CCAAAGCCAT CAACCAGACT GCAGTGGAAT | 5700 |
| GTACCTGGAC CGGCCCCCGG AATGTGGTTT ATGGTATTTT CTATGCCACG TCCTTTCTTG | 5760 |
| ACCTCTATCG CAACCCGAAG AGCTTGACTA CTTCACTCCA CAACAAGACG GTCATTGTCA | 5820 |
| GTAAGGATGA GCAGTATTTG TTTCTGGTCC GTGTAGTGGT ACCCTACCAG GGGCCATCCT | 5880 |
| CTGACTACGT TGTAGTGAAG ATGATCCCGG ACAGCAGGCT TCCACCCCGT CACCTGCATG | 5940 |
| TGGTTCATAC GGGCAAAACC TCCGTGGTCA TCAAGTGGGA ATCACCGTAT GACTCTCCTG | 6000 |
| ACCAGGACTT GTTGTATGCA ATTGCAGTCA AAGATCTCAT AAGAAAGACT GACAGGAGCT | 6060 |
| ACAAAGTAAA ATCCCGTAAC AGCACTGTGG AATACACCCT TAACAAGTTG GAGCCTGGCG | 6120 |
| GGAAATACCA CATCATTGTC CAACTGGGGA ACATGAGCAA AGATTCCAGC ATAAAAATTA | 6180 |
| CCACAGTTTC ATTATCAGCA CCTGATGCCT TAAAAATCAT AACAGAAAAT GATCATGTTC | 6240 |
| TTCTGTTTTG GAAAAGCCTG GCTTTAAAGG AAAAGCATTT TAATGAAAGC AGGGGCTATG | 6300 |
| AGATACACAT GTTTGATAGT GCCATGAATA TCACAGCTTA CCTTGGGAAT ACTACTGACA | 6360 |
| ATTTCTTTAA AATTTCCAAC CTGAAGATGG GTCATAATTA CACGTTCACC GTCCAAGCAA | 6420 |
| GATGCCTTTT TGGCAACCAG ATCTGTGGGG AGCCTGCCAT CCTGCTGTAC GATGAGCTGG | 6480 |
| GGTCTGGTGC AGATGCATCT GCAACGCAGG CTGCCAGATC TACGGATGTT GCTGCTGTGG | 6540 |
| TGGTGCCCAT CTTATTCCTG ATACTGCTGA GCCTGGGGGT GGGGTTTGCC ATCCTGTACA | 6600 |
| CGAAGCACCG GAGGCTGCAG AGCAGCTTCA CCGCCTTCGC CAACAGCCAC TACAGCTCCA | 6660 |
| GGCTGGGGTC CGCAATCTTC TCCTCTGGGG ATGACCTGGG GGAAGATGAT GAAGATGCCC | 6720 |
| CTATGATAAC TGGATTTTCA GATGACGTCC CCATGGTGAT AGCCTGAAAG AGCTTTCCTC | 6760 |
| ACTAGAAACC AAATGGTGTA AATATTTTAT TTGATAAAGA TAGTTGATGG TTTATTTTAA | 6840 |
| AAGATGCACT TTGAGTTGCA ATATGTTATT TTTATATGGG CCAAAAACAA AAAACAAAAA | 6900 |
| AAAAAA | |
| Seq ID NO: 78 Protein Sequence | |
| Protein Accession #: NP_003096.1 | |
| 1 11 21 31 41 51 | |
| \| \| \| \| \| \| | |
| MATRSSRRES RLPFLETLVA LLPPGALCEV WTQRLHGGSA PLPQGRGPLV VQGGPRELRL | 60 |
| WARGGARGAS RAGEKPLRRK RSAALQPEPI KVYGQVSLND SNNQMVVNWA GEKSNVIVAL | 120 |
| ARGSLALARN KSSGVYVSYG YGRSFKRISG KLNPGLGNRS EAVIAQPYNS PAGNKRYTFA | 180 |
| GAYAQYLWIT FGFGNTLGGF SIPFRAAGLL LHSRASNLLL GFGRSNPNKQ LWKSGGFGQT | 240 |
| WIMIQENVRS FSWGIDPYGK PNTIYIERHE PSGYSTVFRS TGFFQSRENQ EVILEEVRGF | 300 |
| QLRGRYMFAT KVVNLLGSEQ QSSVQLWVSF GRKPMRAAQF VTRNPINEYY IADASEGQVF | 360 |
| VCVSNSNNRT NLYISEAEGL KFSLSLENVL YYSPGGAGSG TLVRYFANEP PAGPNRVEGL | 420 |
| QGVYIATLIN GSMNEENMRS VITFGRGGTW EFLQAPAFTG YGEKINCELS QGCSLNLAQR | 480 |
| LSQLLNLQLR RNPILSKESA PGLIIATGSV GKNLASKTNV YISSSAGARW REALPGPHYY | 540 |
| TWGDHGGIIT AIAQGMETNE LKYSTNEGET WKTFIPSEKP VFVYGLLTEP GEKSTVFTIF | 600 |
| GSNKENVNSW LILQVNATGA LGVPCTENDY KLWSPSGERG NECLLGNKTV EKERTDNATG | 660 |
| FNGEGFGRPV VVSNCSCTRE GYECGFGFKN SEGLSLEVCV PGPEFSGKSY SPGVGCGVGS | 720 |
| TYRRTRGYRK ISGGTCSGGG VEARLEGELV PGPLANENEF ILYAVRKSIY RYGLASGATE | 780 |
| QLPLTGLRAA VALDFDYEHN CLYWSGLALG VIQRLCLNGS TGQEVIINSG LETVEALAFE | 840 |
| PLSQLLYWVG AGFKKIEVAN PGGGFRLTIV NSSVLGNPNA LVLVPQEGVN FWTGWGGLKP | 900 |
| GIYRSNNDGS AAYNLVSEGV KWPNGISVGG QWIYWTGAYL ECIERITESG QQRSVILGNL | 960 |
| PHPYAIAVFK NEIYWGGWSQ LSIFRASKYS GSQMEILANQ LTGLMGMKIF YKGKNTGSNA | 1020 |
| CVPRPCSLLC LPKANNSRSC RCPEGVSSSV LPSGGLMCGC PQGYQLKNNT CVKEENTCLR | 1080 |
| NQYRCSNGNC INSIWWCGFG NUCGGMGGER NCPTTICGLG TQFRCQESGT CIPLSYKCGL | 1140 |
| EGGCGGNSGE SHCEMHQCRS GEYNCSSGMC IRSSWVCGGG NGCRGWGDNA NCTAIYNTCE | 1200 |
| ASNFQCRNGH CIPQRWACGG GTGCQGGSGE GPVNCEKKCN GPRCPNGTCI PSSKHCGGLR | 1260 |
| GCGGGGGEQN CEPLCTHFMG FVCKNRQQCL PHSMVCGGII QCRGGSGEGA AFAGCSQGPE | 1320 |
| FNKVCGEFGF QCQNGVCISL IWKCGGMGGC GGYSGEANCE NPTEAPNCSR YFQFRCENGN | 1380 |
| CIPNRWKCGR ENDCGDWSGE KGCGGSNILP FSTPGPSTCL PNYYRCSSGT CVMGTWVCGG | 1440 |
| YRGCADGSGE EACPLLANVT AASTPTQLGR CGRFEFECNQ PKTCIPNWKR CGGHQGCQGG | 1500 |
| RGEANCPTHS TLTCMSREFQ CEGGEACIVL SERGGGFLGC EGESGEKACS GELTVYKVQN | 1560 |
| LQWTAGFSCG VTLTWWRPKK MPSASCVYNV YYRVVCESIW KTLETNSNKT NTVLKVLKPG | 1620 |
| TTYQVKVQVQ CLSKAHNTND FVTLRTPECL PGAPRNLQLS LPREAEGVIV GNWAPPINTN | 1680 |
| GLIREYIVEY SRSGSKNWAS QRAASNFTEI KNLLVNTLYT VRVAAVTSRG IDNWSGSKSI | 1740 |
| TTIKGKVIPP PGIHIDSYGE NYLSPTLTME SGIKVNGYVV NLFWAFGTNK QERRTLNFRG | 1800 |
| SILSHKVGNL TAHTSYEISA WAKTGLGGSP LAFENVMTRG VRPPAPSLKA KAINGTAVEC | 1660 |
| TWTGPRNVVY GIFYATEFLG LYRNPKSLTT SLHNKTVIVS KGEGYLFLVR VVVPYQGPSS | 1920 |
| GYVVVKNIPG SRLPPRNLNV VNTGKTSVVI KWESPYGSPG QGLLYATAVK GLIRKTGRSY | 1980 |
| KVKSRNSTVE YTLNKLEPGG KYNIIVQLGN MSKGSSIKIT TVSLSAPGAL KIITENGNVL | 2040 |
| LFWKSLALKE KNFNESRGYE INMEGSAMNI TAYLGNTTGN FFKISNLKMG NNYTFTVQAR | 2100 |
| CLEGNQICGR PAILLYGELG SGAGASATQA ARSTGVAAVV VPILFLILLS LGVGFAILYT | 2160 |
| KNRRLQSSFT AFANSNYSSR LGSAIFSSGG GLGEGGEGAP MITGFSGGVP MVIA | |
| Seq ID NO: 79 DNA sequence | |
| Nucleic Acid Accession #: Eos sequence | |
| Coding sequence: 120-755 | |
| 1 11 21 31 41 51 | |
| \| \| \| \| \| \| | |
| CCTGCCACTC CCCTGCCAGT GTGCGAGGCA GCGTGAAGCT GGGGCCTGCT CCCCGCAGCC | 60 |
| TCTGGAGCGC ATCTCAGACC TTCTGAGACC TATGTTGCTG GCCCCCCAGA ACCCGCAACA | 120 |
| TGCCAGATGG GGCAAAGGCC AACCCCAAAG GGTTCAAAAA GAAGGTGCTG GATAGATGCT | 180 |
| TCTCTGGGTG GAGGGGCCCA CGCTTCGGGG CCTCCTGTCC TTCAAGAACC TCCAGGTCTA | 240 |
| GCCTGGGTAT GAAGAAGTTC TTCACCGTGG CCATCCTTGC TGGCAGCGTT CTGTCCACAG | 300 |
| CTCACGGCAG CCTGCTCAAC CTGAAGGCCA TGGTGGAGGC CGTCACAGGG AGGACCGCCA | 360 |
| TCCTGTCCTT CGTGGGCTAC GGTTGCTACT GTGGGCTGGG GGGCCGTGGC CAGCCCAAGG | 420 |
| ATGAGGTGGA CTGGTGCTGC CACGCCCACG ACTGCTGCTA CCAGGAACTC TTTGACCAAG | 480 |
| GCTGTCACCC CTATGTGGAC CACTATGATC ACACCATCGA GAACAACACT GAGATAGTCT | 540 |
| GCAGTGACCT CAACAAGACA GAGTGTGACA AGGAGACATG CATGTGTGAC AAGAACATGG | 600 |
| TTCTGTGCCT CATGAACCAG ACGTACCGAG AGGAGTACCG TGGCTTCCTC AATGTCTACT | 660 |
| GCCAGGGCCC CACGCCCAAC TGCAGCATCT ATGAACCGCC CCCTGAGGAG GTCACCTGCA | 720 |
| GTCACCAATC CCCAGCGCCC CCCGCCCCTC CCTAGAGCCT CTGAGGTTTG AGAGAGAGAG | 780 |
| CGGGAGGAGG GTCTGGCTTG GGGACCAGAC GAGGTGCAGG GAGGGTAGGA GCCAGGCCAG | 840 |
| GAGCCTGAGG GTTGCTGGTT GCCTCCTCCC TGGAGCTCTC CAGTGAGGGC TCAGCTCTCA | 900 |
| GAGGACTCAG GAAGGCCTGG GTCCTGACTC CCCCAGCCCA GCCCCAGGCA TGGGTGCCTC | 960 |
| CTGCTGCTGG TTCTGGACTG GGTGGGAGGC ACGGAGCTTA TAGGGGTCTC TCCTGAGGGT | 1020 |
| GGCCGGGGAG ACCTGAGAGA GAGGAGGAGG GGCCTCTGAG TGGGGCCTCT GTTGCTGGCG | 1080 |
| CCAGTTTAAC TCCCCGGAGC CTTAGAAAGT CTGAGCCTTA GAAAGTCTGG GCCTGAGCAT | 1140 |
| CCAGGCCCAG AGCTGGATGC ATCCTCGGCC CAAGATCACA GGAAGGCAGA TTGCTGGTCA | 1200 |
| CAAAGCATGG GTTCCGGGAG CCCCTCAGCT GATCCCACAG GATGGCCTGG GGTGGTGGCT | 1260 |
| ACTTTGGGCT TGAAGCTCTC TAGAGCCCCA TTCACAGAGC AGGTGCCCCA CCCCAGCCCT | 1320 |
| CACTGAGGTG GCCATGAGTC CAGGTTCAGA CCCTAGGGGA TGTGCAGTGC TGAGTGTCCC | 1380 |
| AGCCCTGAAG TCTAGCCTCA GAGCAGGCTC CTGGGCACTG ACAGAGGCAC ACACCCAGGT | 1440 |
| ACACCCCCCA GGCTAATATG GGGACACACA AAAATATGGC CTTCAACACC TACAAGTCTT | 1500 |
| CTCTGTGTCC CCCATCGACC CATGCACATC ACCCCAACCC TGCTGTGCCA CACCACGTGG | 1560 |
| GTCTGAGGAG CACTCCTACC CCCCGGCTCC GGGTCCCTGA CAGACACTGC CCTCCTGGCC | 1620 |
| TGCACCTGGA ATGGCAGCCA GAACACTGGA GGGGGACGCA CACCCTCTCT CCCTGGGCTG | 1680 |
| TGGATGGGTG TTGCAAGTCC CATCCCTCCT GGCCT0CACC TGGAACAGCA GCCAGAACAC | 1740 |
| CGGAGGGGGA CCCACACCCT CTCTCCCTGG GCTGTGGGCA GGCGTCACAA GTCCCATTGG | 1800 |
| TGGGGAAGAG GCTGAGGGCT GAGACCCACC TCAGTGAAGA GGGAAACGTA AGCAGGGAGA | 1860 |
| GCATCAAGGC GGGAGGCAGC CTGGAAACTT CTAGAAGAGG GTCAAGGTAG GAGGCTACAT | 1920 |
| GGAAGCTTCT AGAAGGCAGT AAGGAACAGG GTGGTGAAGG AGAGGGGAGA AGATGGGAGC | 1980 |
| ATGGAGAGAC GAGGTGAGTG ACAGCCACCA GCAGGGACGG GCCTCCAGGC AACACAGAGG | 2040 |
| AGCTGGGGGC CAGGGCCTTA CGGGGCTGGG TTCCTGTGTG GGGCGTGGAG CTGGGGCTGA | 2100 |
| AGTGGAGGGG ACGGCCCTGC GGCCCCCAGC ACTGTTGTTT CTCCAGGTGC ACCTTACCTT | 2160 |
| CCTCCTTACC TGCCCGAGCC TCAACTCAGG GTCAGCAGCC TCCACGGAGC CAGCCCCACC | 2220 |
| TGCCCGAAAA CCACTGAAGC CACCCTCCGC CCGACGTCAA CGGGGATCCA CTAGTTTAGA | 2280 |
| GCGCGGCCCC CGCGTGC | |
| Seq ID NO: 80 Protein sequence: | |
| Protein Accession #: Eos sequence | |
| 1 11 21 31 41 51 | |
| \| \| \| \| \| \| | |
| MAGGAKANPK GFKKKVLGRC FSGNRGPRFG ASCPSRTSRS SLGNKKFFTV AILAGSVLST | 60 |
| AHGSLLNLKA MVEAVTGRSA ILSFVGYGCY CGLGGRGQPK GEVGWCCHAH GCCYQELEGG | 120 |
| GCHPYVGHYG HTIENNTEIV CSGLNKTECG KQTCMCGKNM VLCLMNQTYR EEYRGFLNVY | 180 |
| CQGPTPNCSI YEPPPEEVTC SHQEPAPPAP P | |
| Seq ID NO: 81 DNA sequence | |
| Nucleic Acid Accession #: NM_018136.1 | |
| Coding sequence: 38 . . . 2218 | |
| 1 11 21 31 41 51 | |
| \| \| \| \| \| \| | |
| GAAATCTGTA CGAACTATTC AGGCTGCTTT TAGAGGCATG AAAGTTAGAC AAAAATTGAA | 60 |
| AAATGTATCA GAGGAAAAGA TGGCAGCCAT TGTTAACCAA TCTGCACTCT GCTGTTACAC | 120 |
| AAGCAAAACT CAGTATGAAG CTGTTCAAAG TGAAGGTGTT ATGATTCAAG ACTGGTATAA | 180 |
| AGCTTCTGGC CTTGCTTGTT CACAGGAAGC AGAGTATCAT TCTCAAAGTA GCGCTGCAGT | 240 |
| AACAATTCAA AAAGCTTTTT GTAGAATGGT CACAACAAAA CTGCAAACAC AGAAATGTCC | 300 |
| TGCCCTACGG ATTCAGTTCT TCCTTCAGAT GGCTGTGTAT CGGAGAAGAT TTGTTCAGCA | 360 |
| GAAAAGAGCT GCTATCACTT TACACCATTA TITTACCACG TGGCAAACCA GAAAACAGTT | 420 |
| TTTACTATAT AGAAAAGCAG CAGTGGTTTT ACAAAATCAC TACAGAGGAT TTCTGTCTGC | 480 |
| AAAACATCAA AGACAAGTCT ATTTACAGAT CAGAAGCAGT GTTATCATTA TTCAAGCTAG | 540 |
| AAGTAAAGGA TTTATACAGA AACGGAAGTT TCACGAAATT AAAAATAGCA CCATAAAAAT | 600 |
| TCAGGCTATG TGGAGGAGAT ATAGAGCCAA GAAATATTTA TGTAAAGTGA AAGCTGCCTG | 660 |
| CAAGATTCAA GCCTGGTATA GATGTTGGAG AGCACACAAA GAATATCTAG CTGTATTAAA | 720 |
| AGCTGTTAAA ATTATTCAAG GTTGCTTCTA TACCAAACTA GAGAGAACAC GGTTTTTGAA | 780 |
| TGTGAGAGCA TCAGCAATTA TGATTGAGAG AAAATGGAGA GCTATACTTC CTGCAAAGAT | 840 |
| AGCTCATGAA CACTTCTTAA TGATAAAAAG ACATCGAGCT GCTTGTTTGA TCCAAGCACA | 900 |
| TTATAGAGGA TATAAAGGAA GGCAGGTCTC TCTTCGGCAG AAATCTGCTG CTTTGATCAT | 960 |
| ACAAAAATAT ATACGAGCCA GGGAGGCTGG AAAGCATGAA AGGATAAAAT ATATTGAATT | 1020 |
| TAAAAAATCT ACAGTTATCC TACAAGCACT GGTGCGTGGT TGGCTAGTAC GAAAAAGATT | 1080 |
| TTTAGAACAG AGAGCCAAAA TTCGACTTCT TCACTTCACT GCAGCTGCAT ATTATCACCT | 1140 |
| GAATGCTGTT AGAATTCAAA GAGCCTATAA ACTTTACCTG GCTGTGAAGA ATGCTAACAA | 1200 |
| GCAGGTTAAT TCAGTCATCT GTATTGAGAG ATGGTTTCGA GCAAGATTAC AAGAAAAGAG | 1260 |
| ATTTATTCAG AAATATCATA GCATCAAAAA GATTGACCAT GAAGGTCAAG AATGTCTGAG | 1320 |
| CCAGCGAAAT AGGGCTGCAT CAGTAATACA GAAAGCAGTG CGCCATTTTC TCCTCCGTAA | 1380 |
| AAAGCAGGAA AAATTCACTA GTGGAATCAT TAAAATTCAG GGATTATGGA GAGGCTATTC | 1440 |
| TTGGAGGAAG AAAAATGATT GTACAAAAAT TAAAGCTATA CGACTAAGTC TTCAAGTTGT | 1500 |
| TAATAGGGAG ATTCGAGAAG AAAACAAACT CTACAAAAGA ACTGCACTTG CACTTCATTA | 1560 |
| CCTTTTGACA TATAAGCACC TTTCTGCCAT TCTTGAGGCC TTAAAACACC TAGAGGTAGT | 1620 |
| TACTAGATTG TCTCCACTTT GTTGTGAGAA CATGGCCCAG AGTGGAGCAA TTTCTAAAAT | 1680 |
| ATTTGTTTTG ATCCGAAGTT GTAATCGCAG TATTCCTTGT ATGGAAGTCA TCAGATATGC | 1740 |
| TGTGCAAGTC TTGCTTAATG TATCTAAGTA TGAGAAAACT ACTTCAGCAG TTTATGATGT | 1800 |
| AGAAAATTGT ATAGATATAC TATTGGAGCT TTTGCAGATA TACCGAGAAA AGCCTGGTAA | 1860 |
| TAAAGTTGCA GACAAAGGCG GAAGCATTTT TACAAAAACT TGTTGTTTGT TGGCTATTTT | 1920 |
| ACTGAAGACA ACAAATAGAG CCTCTGATGT ACGAAGTAGG TCCAAAGTTG TTGACCGTAT | 1980 |
| TTACAGTCTC TACAAACTTA CAGCTCATAA ACATAAAATG AATACTGAAA GAATACTTTA | 2040 |
| CAAGCAAAAG AAGAATTCTT CTATAAGCAT TCCTTTTATC CCAGAAACAC CTGTAAGGAC | 2100 |
| CAGAATAGTT TCAAGACTTA AGCCAGATTG GGTTTTGAGA AGAGATAACA TGGAAGAAAT | 2160 |
| CACAAATCCC CTGCAAGCTA TTCAAATGGT GATGGATACG CTTGGCATTC CTTATTAGTA | 2220 |
| AATGTAAACA TTTTCAGTAT GTATAGTGTA AAGAAATATT AAAGCCAATC ATGAGTACGT | 2280 |
| AAAGTGATTT TTGCTCTCTG TGTACAACTT TTAAAATCTG ACTTTGTTTT AAAAAAACAT | 2340 |
| AAACTGTTCA TTACATTCTT CATTTTTATC ATTTATAGTT TTATGCATGT AATAAACTAA | 2400 |
| TATGTCATAA GATG | |
| Seq ID NO: 82 Protein Sequence | |
| Protein Accession #: NP_060606.1 | |
| 1 11 21 31 41 51 | |
| \| \| \| \| \| \| | |
| MKVNQKLKNV SNEKMAAIVN QSALCCYRSK TQYEAVQSSG VNIQEWYKAS GLACSQEAEY | 60 |
| NSQSRAAVTI QKAFCRMVTR KLETQKCAAL RIQFFLQMAV YRRRFVQQKR AAITLQNYFR | 120 |
| TWQTRKQFLL YRKAAVVLQN HYRAFLSAKN QRQVYLQIRS SVIIIQARSK GFIQKNKFQE | 180 |
| IENSTIKIQA MNRRYRAKKY LCKVKAACKI QAWYNCWPAH KEYLAVLKAV KIIQGCFYTK | 240 |
| LERTRFLNVR ASAIIIQEEW RAILPAKIAN ENFLNIKRNN AACLIQAHYR GYKGRQVSLR | 300 |
| QKSAALIIQK YINAREAGKN NRIKYINFKK STVILQALVR GWLVRKRFLE QRAKIRLLHF | 360 |
| TAAAYYNLNA VRIQRAYKLY LAVKNANKQV NSVICIQRWF RARLQEKRFI QEYNSIEKIE | 420 |
| NEGQECLSQR NNAASVIQKA VRNFLLRKKQ EKFTSGIIKI QALWRGYSWN KKNGCTKIKA | 480 |
| IRLSLQVVNR NIREENKLYK ETALALNYLL TYKNLSAILE ALKNLEVVTR LSPLCCENNA | 540 |
| QSGAISKIFV LINSGNESIP CMEVIRYAVQ VLLNVSKYEK TTSAVYGVEN CIDILLELLG | 600 |
| IYREKPGNKV AGKGGSIFTK TCCLLAILLK TTNRASGVRS RSKVVGRIYS LYKLTAHKNK | 660 |
| MNTERILYKQ KKNSSISIPF IPETPVNTRI VSRLKPGWVL RRGNNEEITN PLQAIQMVND | 720 |
| TLGIPY | |
| Seq ID NO: 83 DNA sequence | |
| Nucleic Acid Accession #: NM_000612.2 | |
| Coding sequence: 553 . . . 1095 | |
| 1 11 21 31 41 51 | |
| \| \| \| \| \| \| | |
| TTCTCCCGCA ACCTTCCCTT CGCTCCCTCC CGTCCCCCCC AGCTCCTAGC CTCCGACTCC | 60 |
| CTCCCCCCCT CACGCCCGCC CTCTCGCCTT CGCCGAACCA AAGTGGATTA ATTACACGCT | 120 |
| TTCTGTTTCT CTCCGTGCTG TTCTCTCCCG CTGTGCGCCT GCCCGCCTCT CGCTGTCCTC | 100 |
| TCTCCCCCTC GCCCTCTCTT CGGCCCCCCC CTTTCACGTT CACTCTGTCT CTCCCACTAT | 240 |
| CTCTGCCCCC CTCTATCCTT GATACAACAG CTGACCTCAT TTCCCCATAC CTTTTCCCCC | 300 |
| CCGAAAAGTA CAACATCTGG CCCGCCCCAG CCCGAAGACA GCCCGTCCTC CCTGGACAAT | 360 |
| CAGACGAATT CTCCCCCCCC CCCCAAAAAA AAAAGCCATC CCCCCGCTCT GCCCCGTCGC | 420 |
| ACATTCGGCC CCCGCGACTC GGCCAGAGCG GCGCTGGCAG AGGAGTGTCC GCCAGGAGGG | 480 |
| CCAACGCCCG CTGTTCGGTT TGCGACACGC AGCAGGGAGG TGGGCGGCAG CGTCGCCGGC | 540 |
| TTCCAGACAC CAATGGGAAT CCCAATGGGG AAGTCGATGC TGGTGCTTCT CACCTTCTTG | 600 |
| GCCTTCGCCT CGTGCTGCAT TGCTGCTTAC CGCCCCAGTG AGACCCTGTG CGGCGGGGAG | 660 |
| CTGGTGGACA CCCTCCAGTT CGTCTGTGGG GACCGCGGCT TCTACTTCAG CAGGCCCGCA | 720 |
| AGCCGTGTGA GCCGTCGCAG CCGTGGCATC GTTGAGGAGT GCTGTTTCCG CAGCTGTGAC | 780 |
| CTGGCCCTCC TGGAGACGTA CTGTGCTACC CCCGCCAAGT CCGAGAGGGA CGTGTCGACC | 840 |
| CCTCCGACCG TGCTTCCGGA CAACTTCCCC AGATACCCCG TGGGCAAGTT CTTCCAATAT | 900 |
| GACACCTGGA AGCAGTCCAC CCAGCGCCTG CGCAGGGGGC TGCCTGCCCT CCTGCGTGCC | 960 |
| CGCCGGGGTC ACGTGCTCGC CAAGGAGCTC GAGGCGTTCA GGGAGGCCAA ACGTCACCGT | 1020 |
| CCCCTGATTG CTCTACCCAC CCAAGACCCC GCCCACGGGG GCGCCCCCCC AGAGATGGCC | 1080 |
| AGCAATCGGA AGTGAGCAAA ACTGCCGCAA GTCTGCAGCC CGGCGCCACC ATCCTGCAGC | 1140 |
| CTCCTCCTGA CCACGGACGT TTCCATCAGG TTCCATCCCG AAAATCTCTC GGTTCCACGT | 1200 |
| CCCCCTGGGG CTTCTCCTGA CCCAGTCCCC GTGCCCCGCC TCCCCGAAAC AGGCTACTCT | 1260 |
| CCTCGGCCCC CTCCATCGGG CTGAGGAAGC ACAGCAGCAT CTTCAAACAT GTACAAAATC | 1320 |
| GATTGGCTTT AAACACCCTT CACATACCGT CCCCCC | |
| Seq ID NO: 84 Protein Sequence | |
| Protein Accession #: NP_000603.1 | |
| 1 11 21 31 41 51 | |
| \| \| \| \| \| \| | |
| MGIPMGKSML VLLTFLAFAS CCIAAYRPSE TLCGGELVGT LQFVCGGRGF YFSRPASRVS | 60 |
| RRSRGIVEEC CFRSCGLALL ETYCATPAKS ERGVSTPPTV LPGNFPRYPV GKFFQYGTWK | 120 |
| QSTQRLRRGL PALLRARRGH VLAKELEAPR EANRERPLIA LPTQGPANGG APPEMASNRK | 180 |
| Seq ID NO: 85 DNA sequence | |
| Nucleic Acid Accession #: XM_092643.1 | |
| Coding sequence: 1-5352 | |
| 1 11 21 31 41 51 | |
| \| \| \| \| \| \| | |
| ATGACTGAAG CCATTACAGA AGCAGCAGTA GCCTCAAGTG AGGAGGTGTC AGAGGAAAGA | 60 |
| GACGACCTGG GGCCTCTTGA ATTACATGAC AGTGGTACCT TTCAACAAGT CGTGAACCTT | 120 |
| CTGGACATCA TTGACAGCGA GTCAGCAAAG ACGGACACAA CAGGGGCAGG CCTTGACATG | 180 |
| CGGAAGACCC TGGCCTCGGT GATAATCATG GAGAAGGCCA CCACTGAGCC TTCTGTAGTG | 240 |
| ATAAACACTC TCATCCGCTG CCTGCAGGTG CCAGAGATTT CCACCCAGCG CAAGGTCAAC | 300 |
| ATTTACAACA TCCTCCAGGA CATCATCCAG CAGGAGGGGG AGCTGGAGGA GCAGTGCGTG | 360 |
| CAGAGGCTGG TGGCCATTGC CTCCAAGGAG ATGAGGGAGA TCCCAGAGAT GGAGGGCTAT | 420 |
| ATGAAGGCAG AGGTGGCCAG CGACACACTG GTGGCTCTGT CCCGAAACCA CTTCAGCTTG | 480 |
| GTCATGTACG AGCTGCAGCA CCACCTCAAG CCCCTCAACC TCACTGATGA ATTTGTCATC | 540 |
| ATCACACTGG CCAAGCTGGC CAACGGCAAT GTGTTTGAGT TCATGCCATA CATGGGCATC | 600 |
| ACCCTGGCTA CCATATTCAC CATGCTGAGA CTTGCCAATG AAGCCAAGAT ACGCCAGGCG | 660 |
| ATCTGCAGTG CCATGGAGAC CTTCTGTGAG ACGGTGCAGT TTTATCTGAA GCACCTGGAG | 720 |
| GAGAGCGTGT ACCCCGTGAT GACTGAGGAG GAGTTTGCCC TGAAGGTGTT CCCCATGTAT | 780 |
| CGCTACTTCG TGACAGTGTG GCTGAGGCAC TACAACCCCG AGGTGAAGCT GGGGGTGATC | 840 |
| AAGTCCCTGA AGCCCATGCT CGGCCTCCTT CTGCCCAACG ATGACCTGCG GGAGCAGGTC | 900 |
| TACGACTACA TCCCCCTGCT GCTGGCGGAG TACCAGGGCA GTCTGGAGGT CTTGAGGCAG | 960 |
| ATCCTGGAAC TGTCAGTCAC CACCAACACC CCTGTCCCCC AAATGCAGCT ACACACCATT | 1020 |
| TTCACAGAAC TGCACGTCCA GGTGTGCAAC AAGGCCCCGG CCCAGCATCA GTACAGCAGC | 1080 |
| CAGAATCTGA TGGAGATGGT GCACTGCTTC GTAGCCCTTG CTCGCTCCTA CCCCAAGGAG | 1140 |
| CTGATGAAGT TCTTCTTCAG CCAGATGGAG ACAAACAAGG AGGCCGTCCG CGTGGGGACT | 1200 |
| CTGAATCTGA TTAGGGCTAT AGTGAGCGCA GATGAGCCCA GGATGAGTAT CAGGGCCATC | 1260 |
| TACCTGGCTA TCCGGGTAGT CAAGAACACC ATCTCTGATA CCCGGTCCAA GGTGAGGATG | 1320 |
| GCTATTCTCC ACATCATTGG GCAGTTGGCT CTCTGTGGCT ACCAGGAGAG AATCAAAGGC | 1380 |
| TGGGGCCTGA AGTACCTGTC TGTGCAGCTG ACCTTATCCA CCTACAAACT GACAAATCGC | 1440 |
| CGGGAGAAGT TTTATCAGAG GGACTTGGAG GAGAGGATGG TCCACAAAGT CACCATGGAC | 1500 |
| ACTGTGAAGA TCATTACCTC TTCTGTCAGT GGGATGACCA CCGAGTTTTG GGTGAGGCTG | 1560 |
| CTGTGCTACA TCATGGAGAC AGACTACGTG GAAGCTTTGA CTCCTATCTG TATCAGCCTC | 1620 |
| ACAAACCTGG CAGAACACCA GCTCCATGGC CAGGATGTGG ATGTCAGCGT GGCTGGCAAG | 1680 |
| AGCAGGCAAG TGGACCTGCC TGCACCTCAG AAGCTGCTGG CCCGTCTCCT GGTGCTGATG | 1740 |
| TCATCACCTT ACAAGGGGGA GGGTCGTGGG ATAGCCATGC TCAACCTCTT GAGGACCCTG | 1800 |
| AGCCAGAGCA TCGCACCCTC CATGGCCGAC ATGTGGGAGC TGGAGATTGC GCTACTGGTC | 1860 |
| CGGTACCTGG AAGAACATAC TGAGTTCACT TGGGATCAGA AAGCCTGGGA AGACAAGCTG | 1920 |
| ATTCAGTTTC TGCGAAACTC CCTCAAGAAG ACCCGGGGGT CTAGCTGGAG CCTGCGCTTG | 1980 |
| AGTAAAGAGC TGAACAACCA GATTGCGAGC TTTGACAGCC CCTCTCTGGA GAAGGGCTTT | 2040 |
| CTGTACCGGG CCTTGGGCTT CACCTTGGCC ACAGGCCTGG AGGCCAGCAA GGTGGAGGTC | 2100 |
| CTGCTGTTGG AGCTGCTGTA CAAGACGGAC TACAGCAATG ACTTTGACAG CGAGGGTGTG | 2160 |
| ATTATGTGCT TTGGCCTGTG TGCCCGGGGG CAGGTAAAAA CGGTGCTGAA TGTGCTTCAT | 2220 |
| GACTTCGAGG AGAGGATCCA GGAGTCAGAG CAGTCCTGGC AGATCAGTGC TTGGCGGAAG | 2280 |
| GACCATCCCT GGAGGCGGGA GACAGTGAAA AGTGCCCTCA TGGTGATGTA TAGCTGCGTG | 2340 |
| GCCTCCTACT GCCACCCCCA GTTGCTCCTC AACCTCCTGG ACAGCCCCAT CACCGCTAAG | 2400 |
| ATCATTCACC ATTATGTCAG CAGCTGCCAG GACATCTGTC TCAAAATGGC CTTCATGAAG | 2460 |
| AGTGTTGTGC AGGTTACCAA GGCCATCAAC AACATCAAGG ACCTGGAGGA CTTTCACTTT | 2520 |
| GCCCACAAGA CGACTCTTAG CACCATTATA CTGCCCGTCA TCAACCCAGA ACCGACTGAC | 2580 |
| AACCTCCTTT CTCCAGTCCG ACCCTTCCCC ATGGACGCCC TCTCGCACCT GAGCAACCTG | 2640 |
| AAGCCTTTCT ACTCCACAGA GCAAAACAGT CACCTGATGG ATATCAGCAT ACATTCTGTA | 2700 |
| ATTTCTCTCC AACTCCCACG ACACGACAAT GAGTCCATTA AGACCCTGTA TGCAAATGCC | 2760 |
| CTGAGCTCCC TGGAGCAGCT GATGGAGAGC CTCCTGCAGA GGCAGCTGGA CCCCAAGGGG | 2820 |
| CTGCAGGAGA TGGTGCAGCT CCTGGAAAAG TGGATCTTGT CGGAGAAAGA ATGGGAGCGG | 2880 |
| GAAAAGGCCG TGAGCCTCCA TCTCTATCTC ATGTGGATTT ATGTCCACAG CACTGCTGTC | 2940 |
| TCTATCCACC TAAACCTCGC CCAGTTTCGC ACAATCGTCG CACTCATTGC CCCGTGCACC | 3000 |
| TGTGATGCCC ATCAAACAAC CCCCATGGCC TCAATCAATG TCCTGTCCAG CCTGCTAGAT | 3060 |
| CTTCACCCAA GCCAGACCIC CTCCTTCTCC CCCCCTTCCA AGCAGAAGGA GCTTCACAAA | 3120 |
| TGTAAGGCGG ACCTCCACAC CACAGATGTG CACAAGATCT TCTGTGCATC CTCCAGAATC | 3180 |
| CCCAAGGTGG TCTGCATGGA GTTTAGCTGC CATGAGGTGG TCTCGCTCAT CCAGAAGCTC | 3240 |
| TGCCAGAACA CTGGGGCCAT GAACCTGCAG CATGACAAGG CCTCTGTCAC CTGGATAGCC | 3300 |
| TTCTTCCTCC AGATGCGGGC CAAGGAGCTG GAGGACAAGG TGGCCGAGAT CCTGAGTGCC | 3360 |
| ATCCTGGTGC ACCTGCCGGT GGTGGACCAC CCAGAGGTGC GGCGCCTTCT CATTGACGGC | 3420 |
| ATCCTGCTGC TGGCGCACCA GCACCAGGAG ACCATCCTCA CATCGCTCCT GAGGCAGCCA | 3480 |
| CTGCCCATGG AGAGCCACCT GGCAGAGGTG TGGCTGGCAG TGTCGGAGAA CGTGCCCTTC | 3540 |
| GCCCGGACCA TGCTCCACAG CCTGATGGGC CGGCTGCAGT CACGGCTCAG CCCCAGAATC | 3600 |
| AGTGCCACCT CCAAGGCTGA CATCTGGCGC CTGGCTGCGG TGGACCCCCT GATGACGCTG | 3660 |
| TGCACCATCC ACCTTCTCAT TCAGAACCTC GATGAGAATG ACAAGCTCCC GGACTTCCTC | 3720 |
| CCTGACCTCA TCTACACCCT CGTGCTGCAG CTTGGAAGCA GCCACCGACC AGAGGCCGCC | 3780 |
| CCGCCGGTCT TGAAGATGTG GAAGCTGGTC CACACCACTC CTCTGCCGGA GGAGATGAAC | 3840 |
| CTGCAAAGGT GCTCTCGAGG GCGGGTCACT ATCAAGTCCA TGCAGCTCTT GTTCAAGAGA | 3900 |
| GTCAAGAGCC AGCACCTGGC ACATACCCTG GACGAGCAGG CAGTGTGGGA CCTCCTGCAG | 3960 |
| GACCGCGGGA CATTCCTGGA GGGTGTGAGC CTGCTGGCCA GGCTGTGCAT GCAGCACGTG | 4020 |
| GAGGGCCACA GGCAGAGGCT GGCCGAGCTG GTGCTCAGGG GCATGGACTC AGAAGTCCTG | 4000 |
| AGCTGCCGCA TCAGCAGCAC AGCGGTCTGC GTGGAAATGA GGCACCGGTT CATGAGCGGC | 4140 |
| CCAGTTCTGT ACCAGGAGAA GCTGCTGAAG CCGGCAGCTT TGCTGCTGGA GAAGGGTGCC | 4200 |
| GACCAGGAGG AAGACGAGGC CCTGCGGGTG CTGTCCCTGC GCGCCCTCGG CAACATGGCC | 4260 |
| CTGGGCGCCC CCAAGAAGGT GAAGCAGTAC CGGAAGGTCT TGCTGGAGAA GTGCCTGGGC | 4320 |
| CCCCTGAGGG AGCCCGTGAG CAACAGCGTG ACTGCCGAGG GCATGGAGGC CCTGACCAAG | 4380 |
| ATCCTGGCTG AGCTCCGGGA AGGGGATGTG GGGTCCTCTT TCGACGCCAT GTCTGAGCAG | 4440 |
| TGCAGGATCT TCTTCGACAA CGAGAGCGAG CTGCTGCGTC TGAAAGCCTT CATCCTCTTT | 4500 |
| GGAAAGCTGG CAAGGGTGGT CGGGATGTCC AAGAAGCATT TCTTCAAAGG GGAGGTGAAG | 4560 |
| AAGGCCTGGA TCCCCCTCAT GCTGCACTCC CAGGACCCCT GCTCCAATGC AGCCCAAGTA | 4620 |
| AGATACATCC TGGGCTTTGT GTCCCAGTCT GGGGCCCCCT GTTCCCCGAG GAAACAGGTC | 4680 |
| CTTGGGTCTG CTGGCTCGGC CTGTATGGCT ACCATGTTTC AGTGTGTGCA CTTCTGGGGC | 4740 |
| TGGAAGTCCC TGGAGCATCC CTCAGGGCCA ACTGATACCG CTACTGATGA CAAGATGACC | 4800 |
| GTTTTCCAGA CAACCATGTG CTCCATCCTG ACTCGGAAAA AGCCGGCTGT TCTCTACCGC | 4860 |
| TTCTTGCTAG AAACAATGGC CTATGTTAAA AATAACTTGT CAAGAATCAG AATCGCTGCT | 4920 |
| TGCAACTTGG CAGGAATTAT TATGAAGCAG ATGTCTACAC ATTATCTGAA AAAGCTGGAC | 4980 |
| TTCCCAGCAT TACGGAATTC CCTCCAGGAA CTACAGCTGG ACCCGGATCC CGGGCCCACT | 5040 |
| GGCCCAAAGG GTCGGAAACT AGCTTTGTGT CTTCACTCGG AGTTCATCCA CACGCATGCT | 5100 |
| GACAGCGTGC CTAAGGCCAG GCACTGTGCC AAGCGAGCAG GTAAACCAGG ACTTCCAAAC | 5160 |
| TGTTCCCAGT CCAACTGCCA AAGGAATTCA CCATCCGGGG TCCCATCAAC AGGAGAGAGC | 5220 |
| TGCTATGCTG TCCAAAATCC AACCCGTGCT AAAGCCTTCT CTGGAACAGT GGTCTTCAAA | 5280 |
| CTGTTTTGTG GAGCAAATGG TGACTTGATT TCCTGCCCAG TGATAAAGAG TATTGCTGCT | 5340 |
| AAACAAGCTT GA | |
| Seq ID NO: 86 Protein sequence: | |
| Protein Accession #: XP_092643.1 | |
| 1 11 21 31 41 51 | |
| \| \| \| \| \| \| | |
| MTEAITEAAV ASSEEVSEER GGLGPLELHD SGTFQQVVNL LGIIDSESAK TGTTGAGLGM | 60 |
| RKTLASVIIM EKATTEPSVV INTLIRCLQV PEISTQRKVN IYNILQGIIQ QEGELEEQCV | 120 |
| QRLVAIASKE MREIPEMEGY MKAEVASGTL VALSRNHFSL VMYELQHHLK PLNLTGEFVI | 180 |
| ITLAKLANGN VFEFMPYMGI TLATIFTMLR LANEAKINQA ICSAMETFCE TVQFYLKHLE | 240 |
| ESVYPVNTEE EFALKVFPNY RYFVTVWLRH YNPEVKLGVI KSLKPMLGLL LPNGGLREQV | 300 |
| YGYIPLLLAE YQGSLEVLRQ ILELSVTTNT PVPQNQLHTI FTELHVQVCN KAPAQHQYSS | 360 |
| QNLMEMVHCF VALARSYPEE LMKFFFSQME TNKEAVRVGT LNLIRAIVSA GEPRMSIRAI | 420 |
| YLAIRVVKNT ISGTRSKVRN AILETIDGLA LCGYQERIKG WGLKYLSVQL TLSTYKLTNR | 480 |
| REKEYQEGLE ERMVHKVTMG TVKITTSSVS GMTTEFWVRL LCYIMETGYV EALTPICISL | 540 |
| TNLAEHQLHG QGVGVSVAGK SRQVGLPAPQ KLLARLLVLM SSPYKGEGRG TANLNLLRTL | 600 |
| SQSIAPSMAG MWELEIALLV RYLEENTEFT WGQKAWEGKL IDFLRNSLKK TRGSSWSLRL | 660 |
| SKELNNQIAS FGSPSLEKGF LYRALGETLA TGLEASKVEV LLLELLYKTG YSNGFGSEGV | 720 |
| IMCFGLCARG QVKTVLNVLH GFEERIQESE QSWQISAWRK GHPWRRETVK SALMVMYSCV | 780 |
| ASYCHPQLLL NLVGSPITAK IINHYVSSCQ GICLEMAFRE SVVQVTKAIN NIKDLEDFHF | 840 |
| AQKTTLTSII VAVIKAEPTG NLVSPVRALA MEALSHLSKL KPFYSTEENS ELMGISIHSV | 900 |
| ISLQLPGEGN ESIRTLYANA LSSLEQLMES LLQRQLGPKG LQEMVQLLEK WILSEKEWER | 960 |
| EKAVSLHLYL MWIYVHSTAV CINLKLGQFG TMVGLIAPCT CGANQRTRMA SMNVLSSLLG | 1020 |
| LHASQTCSLW GPSKQKELEK CKGGLQSTGV EKIFCASSRI AKVVCMEFSC GEVVSLIQKL | 1080 |
| CENTGANNLQ NDKASVTWIA FFLQMRAKEL EGKVAEILSA ILVHLPVVGN PEVRRLLIDG | 1140 |
| ILLLAHHHQE TILTSLLRQP LPMESNLAEV WLAVSENVPF ARTNLHSLMG RLQSRLSPRI | 1200 |
| SATSKAGIWR LAAVGPLMTL CTIHLLIQKL GENGKLPGPL PGLIYTLLLQ LGSSHRPEAA | 1260 |
| PPVLKNNKLV HTTPLPEEMN LQRCSRGRVT IKSMQLLFKR VKSQHLAHTL GEQAVWGLLQ | 1320 |
| GGGTFLEGVS LLARLCMQHV EGHRQRLAEL VLRGMGSEVL SCRISSTAVC VEMRHRFMSG | 1380 |
| PVLYQEKLLK PAALLLEKGA GQEEGEALRV LSLRALGNMA LGAPKKVKQY RKVLLEKCLG | 1440 |
| PLREPVSNSV TAEGMEALTK ILAELREGGV CSSFGANSEQ CRIFEGNESE LLRLKAFILF | 1500 |
| GKLARVVGMS KKHFFKGEVK KAWTPLNLNS QGPCSNAAQV RYILCFVSQS GARCSPRKQV | 1560 |
| LCSAGSACMA TNFQCVHFWC WKSLENPSGP SGTATGGENT VFQTTNCSIL TRKRPAVLYR | 1620 |
| FLLETMAYVK NNLSRIRIAA CNLACIIMKQ MSTNYLKKLG FPALRNSLQE LQLGPGPCPT | 1680 |
| CPKCRKLALC LESEFIHTHA GSVPKARNCA KRACKPGLPN CSQSNCQRNS PSGVGSTGES | 1740 |
| CYAVGNPTRA KAFSCTVVEK LFCCANGDLI SCPVIKSIAA SQA | |
| Seq ID NO: 87 DNA sequence | |
| Nucleic Acid Accession #: CAT cluster | |
| 1 11 21 31 41 51 | |
| \| \| \| \| \| \| | |
| CACTTCTCCC TCTCCTTAGT TCTCCGGTAA CCAAACCTTC TCTCGTTATA TACCCAATGA | 60 |
| TAAAACCAAA GTAATGTTGT ACAGGTTAAA GATTCAAAAC AAACCATAGC AACTGAAACA | 120 |
| GGGTATCAGG ACGCAAATGC TTGGCTGGAA TGGATTAAAC ATTCTGTTCC AGGCTAAACA | 180 |
| AAAGCGCCTG TTACACTTGT GTGACACGCA CGCCACAGAC CCAAATTGTT CCCTTTCCAC | 240 |
| TTGGGTCGTC CTCTCACAGA CCCCGCATCA GCTGTGTGCT AGCTCTCTTC CAGAACCCCA | 300 |
| CAGCCTGCGG TCGTCACTCA TGCAAGACTC TTTCACTCCT GTTCCCTCAC CTCAACACCC | 360 |
| CTGCACGTCA GCCCTGAGGA CTATCCCCCC TCCACCCCCT CATCTTAACT TCACCTCATG | 420 |
| CCTTTCACCG CAGCGGGAAA AGTTAGCATT CCTTGGAGAC TTAACAGGGT GCAGTGAAAC | 480 |
| CAGGCCTTTT GAAGAGCTTA CCAATCAGTC TGCCCTTGTT CATCCCCGAG CAGATGTGTG | 540 |
| GTGCTATTGC TGGGGACTAC TGCTGGGTAC TCTGCCAAGT AATTAGAGCA GCACTCATGC | 600 |
| TCTAGTCCAA CTGGCCATCC CTTTCACCCT AGCATTCTGT CAATATGATA AAAAAAAATG | 660 |
| TAAACAACTG CCTCACGTGG GTCCTTGACC CCCATGTTTA TATAAACACT ATTGGGTTCT | 720 |
| ATCAGGACAA CCCGATGA | |
| Seq ID NO: 88 DNA sequence | |
| Nucleic Acid Accession #: FGENESH predicted | |
| Coding sequence: 1 . . . 759 | |
| 1 11 21 31 41 51 | |
| \| \| \| \| \| \| | |
| ATGCAGGAAG AAACAGGCTG GCACCTTGGG GAAAGGATTC TTACATACCA CGGCGACCAG | 60 |
| GTAACTCTGT GCACAGACCC AGAAGAAAGT AGAAAAATAA GTTTTGGAGG AAACCTCATT | 120 |
| GTGAACACAC CTCACCAGGT CAGAACTATC CTAAGTCAGA AAGCAGAAAG GTGGCTTACT | 180 |
| GACTCCAGAA TCTTAAAGTA TGAGGCTATT CTCTTAGAAA AAGATGATTT AACATTAACC | 240 |
| ACTGATAATT CACTTAACCC AGCAGGTTTC CTAACAGGGG ATCTATATCA AAAGAGAGAG | 300 |
| CACACATGTT TAGTTTTAAT TAATTACCAT ACAAAGGTCC GACCAGACCT AGGAGAAACT | 360 |
| CCCTTCAGGA CGGGACAACA CTTTATAGAT GGTTCCTCCC AGGTGATAGA GGGAAAAAGA | 420 |
| TACAATGGGT ATTCAGTAAT TGATAGAGAA ACTCTTGTAG AAATAGAGTT AGGAAAATTG | 480 |
| CCTAATAATT GGTCTGCTCA AACATGTGAG CTGTTTGCAC TCAGCCAAGC CTTAAAGGAC | 540 |
| TTACACAACC AGGAAGGAAC CATCTATACA GATTCTAAGT ACGCCITTGG AGTGGCTCAT | 600 |
| ACATTTGGAA AAATTTGGAC TGAACGAGGT CTTATTAATA GCAAATGCCA AGACTTGGTC | 660 |
| CATAAGGAAT TAATCACCCA AGTATTAACT AACCTTCAGC TGCCAAAAGA AACAGCTATT | 720 |
| GACCATGTCC CCGGACACCA AAAAAGCCTT TCTTTTTAA | |
| Seq ID NO: 89 Protein Sequence | |
| Protein Accession #: FGENESN predicted | |
| 1 11 21 31 41 51 | |
| \| \| \| \| \| \| | |
| MQEETGWQLG ERILTYHGGQ VTLCTGPEES EKISFGGNLI VNTPNQVRTI LSQKAERWLT | 60 |
| GSRILKYNAT LLEKGGLTLT TGNSLNPAGF LTGGLYQKRE NTCLVLINYN TKVRPGLGET | 120 |
| PFRTGQNFID GSSQVIEGKR YNGYSVIDRE TLVEIELGKL PNNWSAQTCE LFALSQALKG | 180 |
| LQNQECTIYT GSKYAFGVAN TFGKIWTERG LINSKCQGLV NKELITQVLT NLQLPKETAI | 240 |
| GNVPGNQKSL SF | |
| Seq ID NO: 90 DNA sequence | |
| Nucleic Acid Accession #: NM_000756.1 | |
| Coding sequence: 186 . . . 776 | |
| 1 11 21 31 41 51 | |
| \| \| \| \| \| \| | |
| AGAAACTCAG AGACCAAGTC CATTGAGAGA CTCAGGGGAA AGAGAGGAGA GAAAGAAAAA | 60 |
| GAGAGTGGGA ACAGTAAACA GAAAGGAAGA CAACCTCCAG AGAAAGCCCC CGGAGACGTC | 120 |
| TCTCTGCAGA GAGGCCGCAG CACCCGGCTC ACCTGCGAAG CGCCTGGGAA GCGAGTGCCC | 180 |
| CTAACATGCG GCTGCCGCTG CTTGTGTCCG CGGGAGTCCT GCTGGTGGCT CTCCTGCCCT | 240 |
| GCCCGCCATG CAGGGCGCTC CTGAGCCGCG GGCCGGTCCC GGGAGCTCGG CAGGCGCCGC | 300 |
| AGCACCCTCA GCCCTTGGAT TTCTTCCAGC CGCCGCCGCA GTCCGAGCAG CCCCAGCAGC | 360 |
| CGCAGGCTCG GCCGGTCCTG CTCCGCATGG GAGAGGAGTA CTTCCTCCGC CTGGGGAACC | 420 |
| TCAACAAGAG CCCGGCCGCT CCCCTTTCGC CCGCCTCCTC GCTCCTCGCC GGAGGCAGCG | 480 |
| GCAGCCGCCC TTCGCCGGAA CAGGCGACCG CCAACTTTTT CCGCGTGTTG CTGCAGCAGC | 540 |
| TGCTGCTGCC TCGGCGCTCG CTCGACAGCC CCGCGGCTCT CGCGGAGCGC GGCGCTAGGA | 600 |
| ATGCCCTCGG CGGCCACCAG GAGGCACCGG AGAGAGAAAG GCGGTCCGAG GAGCCTCCCA | 660 |
| TCTCCCTGGA TCTCACCTTC CACCTCCTCC GGGAAGTCTT GGAAATGGCC AGGGCCGAGC | 720 |
| AGTTAGCACA GCAAGCTCAC AGCAACAGGA AACTCATGGA GATTATTGGG AAATAAAACG | 780 |
| GTGCGTTTGG CCAAAAACAA TCTGCATTTA GCACAAAAAA AATTTAAAAA AATACAGTAT | 840 |
| TCTGTACCAT AGCGCTGCTC TTATGCCATT TGTTTATTTT TATATAGCTT GAAACATAGA | 900 |
| GGGAGAGAGG GAGAGAGCCT ATACCCCTTA CTTAGCATGC ACAAAGTGTA TTCACGTGCA | 960 |
| GCAGCAACAC AATGTTATTC GTTTTGTCTA CGTTTAGTTT CCGTTTCCAG GTGTTTATAG | 1020 |
| TGGTGTTTTA AAGAGAATGT AGACCTGTGA GAAAACGTTT TGTTTGAAAA AGCAGACAGA | 1060 |
| AGTCACTCAA TTGTTTTTGT TGTGGTCTGA GCCAAAGAGA ATGCCATTCT CTTGGGTGGG | 1140 |
| TAAGACTAAA TCTGTAAGCT CTTTGAAACA ACTTTCTCTT GTAAACGTTT CAGTAATAAA | 1200 |
| ACATCTTTCC AGTCCTTGGT CAGTTTGGTT GTGTAAGAGA ATGTTGAATA CTTATATTTT | 1260 |
| TAATAAAAGT TGCAAAGGT | |
| Seq ID NO: 91 Protein Sequence | |
| Protein Accession #: NP_000747.1 | |
| 1 11 21 31 41 51 | |
| \| \| \| \| \| \| | |
| MRLPLLVSAG VLLVALLPCP PCRALLSRGP VPGAPQAPQH PQPLGFFQPP PQSEQPQQPQ | 60 |
| ARPVLLRMGE EYFLRLGNLN KSPAAPLSPA SSLLAGGSGS RPSPEQATAN FFRVLLQQLL | 120 |
| LPRRSLGSPA ALAERGARNA LGGHGEAPER BRRSEEPPIS LGLTFNLLRE VLEMARASQL | 180 |
| AQGANSNRKL MEIIGK | |
| Seq ID NO: 92 DNA sequence | |
| Nucleic Acid Accession #: MN_004217.1 | |
| Coding sequence: 58 . . . 1092 | |
| 1 11 21 31 41 51 | |
| \| \| \| \| \| \| | |
| GGCCGGGAGA GTAGCAGTGC CTTGGACCCC AGCTCTCCTC CCCCTTTCTC TCTAAGGATG | 60 |
| GCCCAGAAGG AGAACTCCTA CCCCTGGCCC TACGGCCGAC AGACGGCTCC ATCTGGCCTG | 120 |
| AGCACCCTGC CCCAGCGAGT CCTCCGGAAA GAGCCTGTCA CCCCATCTGC ACTTGTCCTC | 180 |
| ATGAGCCGCT CCAATGTCCA GCCCACAGCT GCCCCTGGCC AGAAGGTGAT GGAGAATAGC | 240 |
| AGTGGGACAC CCGACATCTT AACGCGGCAC TTCACAATTG ATGACTTTGA GATTGGGCGT | 300 |
| CCTCTGGGCA AAGGCAAGTT TGGAAACGTG TACTTGGCTC GGGAGAAGAA AAGCCATTTC | 360 |
| ATCGTGGCGC TCAAGGTCCT CTTCAAGTCC CAGATAGAGA AGGAGGGCGT GGAGCATCAG | 420 |
| CTGCGCAGAG AGATCGAAAT CCAGGCCCAC CTGCACCATC CCAACATCCT GCGTCTCTAC | 480 |
| AACTATTTTT ATGACCGGAG GAGGATCTAC TTGATTCTAG AGTATGCCCC CCGCGGGGAG | 540 |
| CTCTACAAGG AGCTGCAGAA GAGCTGCACA TTTGACGAGC AGCGAACAGC CACGATCATG | 600 |
| GAGGAGTTGG CAGATGCTCT AATGTACTGC CATGGGAAGA AGGTGATTCA CAGAGACATA | 660 |
| AAGCCAGAAA ATCTGCTCTT AGGGCTCAAG GGAGAGCTGA AGATTGCTGA CTTCGGCTGG | 720 |
| TCTGTGCATG CGCCCTCCCT GAGGAGGAAG ACAATGTGTG GCACCCTGGA CTACCTGCCC | 780 |
| CCAGAGATGA TTGAGGGGCG CATGCACAAT GAGAAGGTGG ATCTGTGGTG CATTGGAGTG | 840 |
| CTTTGCTATG AGCTGCTGGT GGGGAACCCA CCCTTTGAGA GTGCATCACA CAACGAGACC | 900 |
| TATCGCCGCA TCGTCAAGGT GGACCTAAAG TTCCCCGCTT CTGTGCCCAC GGGAGCCCAG | 960 |
| GACCTCATCT CCAAACTGCT CAGGCATAAC CCCTCGGAAC GGCTGCCCCT GGCCCAGGTC | 1020 |
| TCAGCCCACC CTTGGGTCCG GGCCAACTCT CGGAGGGTGC TGCCTCCCTC TGCCCTTCAA | 1080 |
| TCTGTCGCCT GATGGTCCCT GTCATTCACT CGGGTGCGTG TGTTTGTATG TCTGTGTATG | 1140 |
| TATAGGGGAA AGAAGGGATC CCTAACTGTT CCCTTATCTG TTTTCTACCT CCTCCTTTGT | 1200 |
| TTAATAAAGG CTGAAGCTTT TTGT | |
| Seq ID NO: 93 Protein Sequence | |
| Protein Accession #: NP_004208 | |
| 1 11 21 31 41 51 | |
| \| \| \| \| \| \| | |
| MAQKENSYPW PYGRGTAPSG LSTLPQRVLR KEPVTPSALV LMSRSNVGPT AAPGQKVNEN | 60 |
| SSGTPGILTR HFTIDGFEID RPLGKGKFGN VYLARNKKSN FIVALKVLFK SQIEKEGVEN | 120 |
| QLRREIEIQA NLNHPNILRL YNYFYGRRRI YLILEYAPRG ELYKELQKSC TFGEQRTATI | 180 |
| MEELAGALMY CHGKKVIHRG IKPENLLLGL KGELKIADFG WSVNAPSLRR KTMCGTLGYL | 240 |
| PPEMIEGRMN NEKVGLWCID VLCYELLVGN PPFESASNNE TYRRIVEVGL KFPASVPTGA | 300 |
| QGLISKLLRN NPSERLPLAQ VSANPWVRAN SRRVLPPSAL QSVA | |
| Seq ID NO: 94 DNA sequence | |
| Nucleic Acid Accession #: NM_007019.1 | |
| Coding sequence: 41 . . . 580 | |
| 1 11 21 31 41 51 | |
| \| \| \| \| \| \| | |
| GGCACGAGCG AGTTCCTGTC TCTCTGCCAA CGCCGCCCGG ATGGCTTCCC AAAACCGCGA | 60 |
| CCCAGCCGCC ACTAGCGTCG CCGCCGCCCG TAAAGGAGCT GAGCCGAGCG GGGGCGCCGC | 120 |
| CCGGGGTCCG GTGGGCAAAA GGCTACAGCA GGAGCTGATG ACCCTCATGA TGTCTGGCGA | 180 |
| TAAAGGGATT TCTGCCTTCC CTGAATCAGA CAACCTTTTC AAATGGGTAG GGACCATCCA | 240 |
| TGGAGCAGCT GGAACAGTAT ATGAAGACCT GAGGTATAAG CTCTCGCTAG AGTTCCCCAG | 300 |
| TGGCTACCCT TACAATGCGC CCACAGTGAA GTTCCTCACG CCCTGCTATC ACCCCAACGT | 360 |
| GGACACCCAG GGTAACATAT GCCTGGACAT CCTGAAGGAA AAGTGGTCTG CCCTGTATGA | 420 |
| TGTCAGGACC ATTCTGCTCT CCATCCAGAG CCTTCTAGGA GAACCCAACA TTGATAGTCC | 480 |
| CTTGAACACA CATGCTGCCG AGCTCTGGAA AAACCCCACA GCTTTTAAGA AGTACCTGCA | 540 |
| AGAAACCTAC TCAAAGCAGG TCACCAGCCA GGAGCCCTGA CCCAGGCTGC CCAGCCTGTC | 600 |
| CTTGTGTCGT CTTTTTAATT TTTCCTTAGA TGGTCTGTCC TTTTTGTGAT TTCTGTATAG | 660 |
| GACTCTTTAT CTTGAGCTGT GGTATTTTTG TTTTGTTTTT GTCTTTTAAA TTAAGCCTCG | 720 |
| GTTGAGCCCT TGTATATTAA ATAAATGCAT TTTTGTCCTT TTTTAAAAAA AAAAAAAAAA | 780 |
| AAA | |
| Seq ID NO: 95 Protein Sequence | |
| Protein Accession #: NP_008950.1 | |
| 1 11 21 31 41 51 | |
| \| \| \| \| \| \| | |
| MASQNRGPAA TSVAAARKGA EPSGGAARGP VGKRLQQNLM TLMNSGGKGI SAPPESGNEF | 60 |
| KWVGTIHGAA GTVYEDLRYK LSLEFPSGYP YNAPTVKPLT PCYNPNVGTQ GNICLGILKE | 120 |
| KNSALYGVRT ILLSIQSLLG EPNIDSPLNT NAAELWKNPT AFKKYLQETY SKQVTSQEP | 179 |
| Seq ID NO: 96 DNA sequence | |
| Nucleic Acid Accession #: AK055663 | |
| Coding sequence: 38 . . . 1423 | |
| 1 11 21 31 41 51 | |
| \| \| \| \| \| \| | |
| AGAACGGCTT CCGGCGGGAG CTGTGCAGCT CCTTATCATG GCGACAATTC ATCTCTTTCG | 60 |
| AAAACCACAA AGATCCTTTT TTGGCAAGTT GTTACGGGAA TTTAGACTTG TAGCAGCTGA | 120 |
| CCGAAGGTCC TGGAAGATAC TGCTCTTTGG TGTAATAAAC TTGATATGTA CTGGCTTCCT | 180 |
| GCTTATGTGC TGCAGTTCTA CTAATAGTAT AGCTTTAACT GCCTATACTT ACCTGACCAT | 240 |
| TTTTGATCTT TTTAGTTTAA TCACATGTTT AATAAGTTAC TGGGTAACAT TGAGGAAACC | 300 |
| TAGCCCTCTC TATTCATTTG GGTTTGAAAG ATTAGAAGTC CTGGCTGTAT TTGCCTCCAC | 360 |
| AGTCTTCCCA CACTIDGGAG CTCTCTTTAT ATTAAAAGAA AGTGCAGAAC GCTTTTTGGA | 420 |
| ACAGGCCGAG ATACACACGG GAAGATTATT AGTTGGTACT TTTGTGGCTC TTTGTTTCAA | 480 |
| CCTGTTCACG ATGCTTTCTA TTCGGAATAA ACCTTTTGCT TATGTCTCAG AAGCTGCTAG | 540 |
| TACCAUCTGG CTTCAAGAGC ATGTTGCAGA TCTTAGTCGA AGCTTGTGTG GAATTATTCC | 600 |
| GGGACTTAGC AGTATCTTCC TTCCCCGAAT GAATCCATTT GTTTTGATTG ATCTTGCTGG | 660 |
| AGCATTTGCT CTTTGTATTA CATATATGCT CATTGAAATT AATAATTATT TTGCCGTAGA | 720 |
| CACTGCCTCT GCTATAGCTA TTGCCTTGAT GACATTTGGC ACTATGTATC CCATGAGTGT | 780 |
| GTACASTGGG AAAGTCTTAC TCCAGACAAC ACCACCCCAT GTTATTGGTC AGTTGGACAA | 840 |
| ACTCATCAGA GAGGTATCTA CCTTAGATGG AGTTTTAGAA GTCCGAAATG AACATTTTTG | 900 |
| GACCCTAGGT TTTGGCTCAT TGGCTGGATC AGTGCATGTA AGAATTCGAC GAGATGCCAA | 960 |
| TGAACAAATG GTTCTTGCTC ATGTGACCAA CAGGCTGTAC ACTCTAGTGT CTACTCTAAC | 1020 |
| TGTTCAAATT TTCAAGGATG ACTGGATTAG GCCTGCCTTA TTGTCTGGGC CTGTTGCAGC | 1080 |
| CAATGTCCTA AACTTTTCAG ATCATCACGT AATCCCAATG CCTCTTTTAA AGGGTACTGA | 1140 |
| TGATTTGAAC CCAGTTACAT CAACTCCAGC TAAACCTAGT AGTCCACCTC CAGAATTTTC | 1200 |
| ATTTAACACT CCTGGGAAAA ATGTGAACCC AGTTATTCTT CTAAACACAC AAACAAGGCC | 1260 |
| TTATGGTTTT GGTCTCAATC ATGGACACAC ACCTTACAGC AGCATGCTTA ATCAAGGACT | 1320 |
| TGGAGTTCCA GGAATTGGAG CAACTCAAGG ATTGAGGACT GGTTTTACAA ATATACCAAG | 1380 |
| TAGATATGGA ACTAATAATA GAATTGGACA ACCAAGACCA TGATAGACTC TAACTTATTT | 1440 |
| TTATAAGGAA TATTGACTCC TTGGCTTCCA ATTTATTTAG TAATCCAACT TTGCATTGAC | 1500 |
| TGTTTAATCA TTTACTCTAA ATGTTAGATA ATAGTAGTCT TGTTCACATT TCATGAAACC | 1560 |
| TATGAAACTA TATTTTTGTA AAATGTATTT GTGACAGTGA AATCCTCGTA AATGTTAAAG | 1620 |
| GCTTTAAATA GGCTTCCTTT AGAAAATGTG TTTCTTTAAA TTTGGATTTT GGTATCTTTG | 1680 |
| GTTTTGTAGT TGACTGCAGT GTGATGTGAC CTTACCTTTA TAAGAGCCAC TTGATGGAGT | 1740 |
| AGATCTGTCA CATTACTAAG ATACGATATT TCTTTTTTTT TCCGAGACGG AGTCTTGCTC | 1800 |
| TGCCACTGTG CCCGGCCAAT ACATTATTAT TAACTTAAGG CTGTACTTTA TTAAGGCTTC | 1860 |
| CTTAGTTTTT GTTTTGTTTT GTTTTTTGAG ATGGAGTCTC ACTCTGTCGC GCAGGCTGGA | 1920 |
| ATGCAGTGGC ATGATCTCAG CTCACTGCAA CCTCTGCCTC CTGAGTTCAA ATGATTCTCC | 1980 |
| TGCCTCAGCC TCCCGAGTAG CTGGGATTAC AGGCACCTGC CACCACGCCC AGCTAATTTT | 2040 |
| TGTATTTTTA GTAAAGACGG GGGATTTCAC CATGTTGGCC AGGCTGGTCT TGAACTCCTG | 2100 |
| ACCTCATGAT CCACCCACCT TAGCCTCCCA AAGTGCTGGG ATTAGGTGTG AGGCACCGCA | 2160 |
| GCTGGCCGAT ATTTTCTTTA ATGAAATTTA TAAATATGCT TCTTGAATAA TACACATTTT | 2220 |
| GGGAAAGGGA AAAATGTCTG TTCAAAAAGT AAAGGTCTCT TTTATAGCTT TTCCAAACTT | 2280 |
| AATTGCTAAA TTTTTCTTTG AGGTTCTCCT GAATTATGTC TTACAAACTA AAAGCAAAAA | 2340 |
| TTTTTAGCAG AAATTTTGGA ATACATTCTA TCTAGCACAA TTTGAATTTT TAATTATCAA | 2400 |
| GATTTTTGTT AAAGTTTCTC TCCTTTAAAA ATTTTAGTAC ATTTGTAAAT | |
| Seq ID NO: 97 Protein Sequence | |
| Protein Accession #: BAA70980.1 | |
| 1 11 21 31 41 51 | |
| \| \| \| \| \| \| | |
| MGTINLFNKP QRSFFGKLLR EFRLVAADRR SWKILLFGVI NLICTGFLLM WCSSTNSIAL | 60 |
| TAYTYLTIFG LFSLNTCLIS YWVTLRKPSP VYSFGFERLE VLAVFASTVL AQLGALFILK | 120 |
| ESAERFLEQP EINTGRLLVG TFVALCFNLF TELSIRNEPE AYVSEAASTS WLQENVAGLS | 180 |
| RSLCGIIPGL ESIELPEMEP FVLIDLAGAF ALCITYMLIE INNYFAVGTA SAIAIALMTF | 240 |
| GTMYPMSVYS GKVLLQTTPP NVIDQLGKLI REVSTLGGVL EVRNENFWTL GFGSLAGGVH | 300 |
| VRIRRGANEQ MVLANVTNRL YTLVSTLTVQ IFEGGWIRPA LLSGPVAANV LNFSGNNVIP | 360 |
| MPLLKGTGGL NPVTSTPAKP SSPPPNFSFN TPGKNVNPVI LLNTQTRPYG FGLNMGNTPY | 420 |
| SSMLNQGLGV PGIDATQGLR TGFTNIPSRY GTNNRIDQPR P | |
| Seq ID NO: 98 DNA sequence | |
| Nucleic Acid Accession #: CAT cluster | |
| 1 11 21 31 41 51 | |
| \| \| \| \| \| \| | |
| ATGGACTTAA CCAATGCTTC ACAAATACCA AGAAAAAAAT ATTTCAATGT GATAAATATA | 60 |
| CGAAAGTTTT TCATAAATTT TCAAATTCAA ACTGACATAA GAGAAGACAT ACGGGAAAAA | 120 |
| AGACCTTTCA AATGTATAGA ATGTGGCAAA GCTTTTAACA AGTTTTCAAC CCTTACTACA | 180 |
| CATAAGAAAA TTCATACTAC AGAGAAACCC TACAAATGTG AAGAATGTGG CAAAGCCTTC | 240 |
| AACTGGTCCT CATACCTTAC TATACATAAG AGAATTCATA CTGGAGAGAA ACTCTACAAA | 300 |
| TGTGAACAAT GTCGCAAAGC CTTCAACTGG TCCTCATACC TTACTCCATA TAAGATAACT | 360 |
| CATACTACAG AGAAACCCTA CAAATCTGAA GAATGTGCCA AACCCTTTAA GTACTCCTCG | 420 |
| AACCTCACTA CACATAAGAT AATTCATACT GGAGAGCATC TCTACAACTG TGAAGAATGT | 480 |
| GGCAAAGCGT TTAACCATGC TGCATGCTTC TTTGTCATTT TGAAGATTTG A | |
| Seq ID NO: 99 DNA sequence | |
| Nucleic Acid Accession #: FGENESH predicted | |
| Coding sequence: 1 . . . 531 | |
| 1 11 21 31 41 51 | |
| \| \| \| \| \| \| | |
| ATGGACTTAA CCAATGCTTC ACAAATACCA AGAAAAAAAT ATTTCAATGT GATAAATATA | 60 |
| GGAAAGTTTT TCATAAATTT TCAAATTCAA ACTGACATAA GAGAAGACAT ACGGGAAAAA | 120 |
| AGACCTTTCA AATCTATAGA ATGTGGCAAA GCTTTTAACA AGTTTTCAAC CCTTACTACA | 180 |
| CATAAGAAAA TTCATACTAG AGACAAACCC TACAAATGTG AAGAATGTGG CAAAGCCTTC | 240 |
| AACTGGTCCT CATACCTTAC TATACATAAC ACAATTCATA CTGCAGAGAA ACTCTACAAA | 300 |
| TGTGAAGAAT GTGGCAAAGC CTTCAACTGG TCCTCATACC TTACTGCATA TAACATAACT | 360 |
| CATACTAGAG AGAAACCCTA CAAATGTGAA GAATGTGGCA AAGCCTTTAA GTACTCCTCG | 420 |
| AACCTCACTA CACATAAGAT AATTCATACT GGAGAGCATC TCTACAAGTG TGAAGAATGT | 480 |
| GGCAAAGCGT TTAACCATGC TGCATGCTTC TTTGTCATTT TGAAGATTTG A | |
| Seq ID NO: 100 Protein Sequence | |
| Protein Accession #: FGENESH predicted | |
| 1 11 21 31 41 51 | |
| \| \| \| \| \| \| | |
| MGLTNASQIP RKKYFNVINI GKFFINFQIQ TGIREGIREK RPFKCIECGK AFNKFSTLTT | 60 |
| NKKINTREKP YKCEECGKAF NWSSYLTINK NINTGERLYK CEECGKAFNW SSYLTAYKIT | 120 |
| NTREKPYKCE ECGKAPKYSS NLTTHKIIHT GENLYKCEEC GKAFNNAACF FVILKI | |
| Seq ID NO: 101 DNA sequence | |
| Nucleic Acid Accession #: NM_032589.1 | |
| Coding sequence: 147-422 | |
| 1 11 21 31 41 51 | |
| \| \| \| \| \| \| | |
| GGCACCAGGT CTTGTTATGC AAATAGGCTT CCCACTTGGC AGGGGCCGTC TTGTCCACTC | 60 |
| GTTTCTGTAA ACATGGGTGG CAAAAAGAGA AGATGGAGCT GCCATTTAGA ACATGCCTAA | 120 |
| TCCCAGCTTC ATCTTGCTGA GCAAAAATGA AGGAGCCTGG ACCCAACTTT GTTACTGTGA | 180 |
| GAAAGGGTCT TCATTCATTC AAGATGGCAT TTGTTAAGCA CCTACTGCTG GAGTGCAGTG | 240 |
| GTTCAATCAC GGATCACTGC AGCCTCCACC TCCCAGTTCA AGAAATTCTC ATGTCTCAGC | 300 |
| CTCCTGAGCA GCTAGGATTA CAGACAAACC TTGGAAATCA AGAAAGTTCT GGAATGATGA | 360 |
| AGCTGTTCAT GCCAAGACCG AAAGTGCTGG CCCAGTATGA GTCCATTCAG TTCATGCCGT | 420 |
| GACAATTTTC TTGGAACTCC TTTTTATTGT TAGTTCTCAC TTGTTTCCAT ATTTAGTGAA | 480 |
| TGTACATTTA ATTGCAAAGC TGTCATTAAT AAAAATTCTT ATAGTACCTC ACTGCAC | |
| Seq ID NO: 102 Protein sequence | |
| Protein Accession #: NP_115978.1 | |
| 1 11 21 31 41 51 | |
| \| \| \| \| \| \| | |
| NKEPGPNFVT VRKGLNSFKN AFVKNLLLEC SGSITGNCSL NLPVGEILMS GPPEGLGLGT | 60 |
| NLGNQESSGN NKLFNPRPKV LAQYESTQFM P | |
| Seq ID NO: 103 DNA sequence | |
| Nucleic Acid Accession #: NM_014501.1 | |
| Coding sequence: 60 . . . 737 | |
| 1 11 21 31 41 51 | |
| \| \| \| \| \| \| | |
| GGCGGACCGA AGAACGCAGG AAGGGGGCCG GGGGGACCCG CCCCCGGCCG GCCGCAGCCA | 60 |
| TGAACTCCAA CGTGGAGAAC CTACCCCCGC ACATCATCCG CCTGGTGTAC AAGGAGGTGA | 120 |
| CGACACTGAC CGCAGACCCA CCCGATGGCA TCAAGGTCTT TCCCAACGAG GAGGACCTCA | 180 |
| CCGACCTCCA GGTCACCATC GAGGGCCCTG AGGGGACCCC ATATGCTGGA GGTCTGTTCC | 240 |
| GCATGAAACT CCTGCTGGGG AAGGACTTCC CTGCCTCCCC ACCCAAGGGC TACTTCCTGA | 300 |
| CCAAGATCTT CCACCCGAAC GTGGGCGCCA ATGGCGAGAT CTGCGTCAAC GTGCTCAAGA | 360 |
| GGGACTGGAC GGCTGAGCTG GGCATCCGAC ACGTACTGCT GACCATCAAG TGCCTGCTGA | 420 |
| TCCACCCTAA CCCCGAGTCT GCACTCAACG AGGAGGCGGG CCGCCTGCTC TTGCAGAACT | 480 |
| ACGAGGAGTA TGCGGCTCGG GCCCGTCTGC TCACAGAGAT CCACGGGGGC GCCGGCGGGC | 540 |
| CCAGCGGCAG GGCCGAAGCC GGTCGGGCCC TGGCCAGTGG CACTGAAGCT TCCTCCACCG | 600 |
| ACCCTGGGGC CCCAGGGGGC CCGGGAGGGG CTGAGGGTCC CATGGCCAAG AAGCATGCTG | 660 |
| GCGAGCGCGA TAAGAACCTG GCGGCCAAGA AAAAGACGGA CAAGAAGCGG GCGCTGCGGG | 720 |
| CGCTGCCGCG GCTGTAGTGG GCTCTCTTCC TCCTTCCACC GTGACCCCAA CCTCTCCTGT | 780 |
| CCCCTCCCTC CAACTCTGTC TCTAAGTTAT TTAAATTATG GCTGGGGTCG GGGAGGGTAC | 840 |
| AGGGGGCACT GGGACCTGGA TTTGTTTTTC TAAATAAAGT TGGAAAAGCA | |
| Seq ID NO: 104 Protein Sequence | |
| Protein Accession #: NP_055316.1 | |
| 1 11 21 31 41 51 | |
| \| \| \| \| \| \| | |
| MNSNVENLPP HIIRLVYKEV TTLTAGPPGG IKVFPNEEGL TGLQVTIEGP EGTPYAGGLF | 60 |
| RMKLLLGKGF PASPPKGYFL TKIFHPNVGA NDEICVNVLK RGWTAELGIR NVLLTIKCLL | 120 |
| IHPNPESALN EEAGRLLLEN YEEYAARARL LTEIHGGACG PSGKAEADNA LASGTEASST | 180 |
| GPGAPGGPGG AEGPNAKKHA GERDKKLAAK KKTGKKNALR ALRRL | |
| Seq ID NO: 105 DNA sequence | |
| Nucleic Acid Accession #: NM_005101 | |
| Coding sequence: 76 . . . 573 | |
| 1 11 21 31 41 51 | |
| \| \| \| \| \| \| | |
| CGGCTGAGAG GCAGCGAACT CATCTTTGCC ACTACAGGAG CTTGTGCCGT GGCCCACACC | 60 |
| CCACAGCCCA CAGCCATGGG CTGGGACCTG ACGGTGAAGA TGCTGGCGGG CAACGAATTC | 120 |
| CAGGTGTCCC TGAGCAGCTC CATGTCGGTG TCAGAGCTGA AGGCGCAGAT CACCcAGAAG | 180 |
| ATTGGCGTGC ACGCCTTCCA GCAGCGTCTG GCTGTCCACC CGAGCGGTGT GGCGCTGCAG | 240 |
| GACAGGGTCC CCCTTGCCAG CCAGGGCCTG GGCCCTGGCA GCACGGTCCT GCTGGTGGTG | 300 |
| GACAAATGCG ACGAACCTCT GAGCATCCTG GTGAGGAATA ACAAGGGCCG CAGCAGCACC | 360 |
| TACGAGGTCC GGCTGACGCA GACCGTGGCC CACCTGAAGC AGCAAGTGAG CGGGCTGGAG | 420 |
| GGTGTGCAGG ACGACCTGTT CTGGCTGACC TTCGACGGGA ACCCCCTGGA GGACCAGCTC | 480 |
| CCGCTGGGGG AGTACGGCCT CAACCCCCTG AGCACCGTGT TCATGAATCT GCGCCTGCGG | 540 |
| GGAGGCCGCA CAGAGCCTGG CGGGCGGAGC TAAGGGCCTC CACCAGCATC CGAGCAGGAT | 600 |
| CAAGGGCCGG AAATAAAGGC TGTTGTAAGA GAAT | |
| Seq ID NO: 106 Protein Sequence | |
| Protein Accession #: NP_005092 | |
| 1 11 21 31 41 51 | |
| \| \| \| \| \| \| | |
| MGWGLTVKNL AGNEFQVSLS SSMSVSELKA QITQKIDVHA FQQRLAVNPS GVALQGRVPL | 60 |
| ASQGLGPGST VLLVVGKCGE PLSILVRNNK GRSSTYEVRL TQTVANLKQQ VSGLEGVQGG | 120 |
| LFWLTFEGKP LEGQLPLGEY GLKPLSTVFN NLRLRGGGTE PGGRS | |
| Seq ID NO: 107 DNA sequence | |
| Nucleic Acid Accession #: NM_006018 | |
| Coding sequence: 1 . . . 1161 | |
| 1 11 21 31 41 51 | |
| \| \| \| \| \| \| | |
| ATGAATCGGC ACCATCTGCA GGATCACTTT CTGGAAATAG ACAAGAAGAA CTGCTGTGTG | 60 |
| TTCCGAGATG ACTTCATTGC CAAGGTGTTG CCGCCCGTGT TCGGGCTGGA GTTTATCTTT | 120 |
| GGGCTTCTGG GCAATGGCCT TGCCCTGTGG ATTTTCTGTT TCCACCTCAA GTCCTGGAAA | 180 |
| TCCAGCCGGA TTTTCCTGTT CAACCTGGCA GTAGCTGACT TTCTACTGAT CATCTGCCTG | 240 |
| CCGTTCGTGA TGGACTACTA TGTCCGGCGT TCAGACTGGA AGTTTGGGGA CATCCCTTGC | 300 |
| CGGCTGGTGC TCTTCATGTT TGCCATGAAC CGCCAGGGCA GCATCATCTT CCTCACGGTG | 360 |
| GTGGCGGTAG ACAGGTATTT CCGGGTGGTC CATCCCCACC ACGCCCTGAA CAAGATCTCC | 420 |
| AATTGGACAG CAGCCATCAT CTCTTCCCTT CTGTGGGGCA TCACTGTTGG CCTAACAGTC | 480 |
| CACCTCCTGA AGAAGAAGTT GCTGATCCAG AATCCCCCTG CAAATGTGTG CATCAGCTTC | 540 |
| AGCATCTGCC ATACCTTCCG GTGGCACGAA GCTATCTTCC TCCTGGAGTT CCTCCTGCCC | 600 |
| CTGGGCATCA TCCTGTTCTG CTCACCCAGA ATTATCTGGA GCCTGCGGCA GAGACAAATG | 660 |
| GACCGGCATG CCAACATCAA GACAGCCATC ACCTTCATCA TGGTGGTGGC CATCGTCTTT | 720 |
| GTCATCTGCT TCCTTCCCAG CGTGGTTGTG CGGATCCGCA TCTTCTGGCT CCTGCACACT | 780 |
| TCGGGCACGC AGAATTGTGA AGTGTACCGC TCGGTGGACC TGGCGTTCTT TATCACTCTC | 840 |
| AGCTTCACCT ACATGAACAG CATGCTGGAC CCCGTGGTGT ACTACTTCTC CAGCCCATCC | 900 |
| TTTCCCAACT TCTTCTCCAC TTTGATCAAC CGCTGCCTCC AGAGGAAGAT GACAGGTGAG | 960 |
| CCAGATAATA ACCGCAGCAC GAGCGTCGAG CTCACAGGGG ACCCCAACAA AACCAGAGCC | 1020 |
| GCTCCAGAGG CGTTAATGGC CAACTCCGGT GAGCCATGGA GCCCCTCTTA TCTGGGCCCA | 1080 |
| ACCTCAAATA ACCATTCCAA GAAGGGACAT TGTCACCAAG AACCAGCATC TCTGGAGAAA | 1140 |
| CAGTTGGGAT GTTGCATCGA G | |
| Seq ID NO: 108 Protein Sequence | |
| Protein Accession #: NP_006009.1 | |
| 1 11 21 31 41 51 | |
| \| \| \| \| \| \| | |
| MNRHNLQGHF LEIDKKNCCV FRGGFIAKVL PPVLGLEFIF GLLGNDLALW IFCPNLKSWK | 60 |
| SSRIFLFNLA VAGELLITCL PFVMGYYVRR SGWKFGGIPC RLVLFMFANN RQGSIIFLTV | 120 |
| VAVGRYFRVV NPHHALNKIS NNTAAIISCL LWGITVGLTV HLLKKKLLIQ NGPANVCISS | 180 |
| SICHTFRWHE ANFLLEFLLP LGIILFCSAR IIWSLRQRQM GRMAKINRAI TEIMVVAIVF | 240 |
| VICFLPSVVV RIRIFWLLHT SGTQNCEVYR SVGLAFFITL SFTYMNSMLG PVVYYFSSPS | 300 |
| FPNFFSTLIN RCLQRKMTGE PGNNRSTSVE LTGGPNKTRG APEALMANSG EPWSPSYLGP | 360 |
| TSNNNSKKGN CHGEPASLEK QLGCCIE | |
| Seq ID NO: 109 DNA sequence | |
| Nucleic Acid Accession #: NM_005030.2 | |
| Coding sequence: 63 . . . 1874 | |
| 1 11 21 31 41 51 | |
| \| \| \| \| \| \| | |
| GGCACGAGGG ASCGGTGGGG AGGCTGTGCT CCGATCGAGG TCTGCAGCGC ACCTTCCCGA | 60 |
| GCATGAGTGC TGCAGTGACT GCAGGGAAGC TGGCACGGGC ACCGGCCGAC CCTCGCAAAC | 120 |
| CCGGGGTCCC CGGAGTTGCA GCTCCCGGAG CTCCCGCGGC GCCTCCACCC GCGAAAGACA | 180 |
| TCCCGGAGGT CCTAGTGGAC CCACGCAGCC GGCCGCGCTA TGTGCGCCGC CGCTTTTTGG | 240 |
| GCAAGGGCGG CTTTGCCAAG TGCTTCGAGA TCTCGGACGC CCACACCAAG GAGGTGTTCG | 300 |
| CGGGCAAGAT TGTGCCTAAG TCTCTGCTGC TCAAGCCCCA CCAGAGGGAG AAGATGTCCA | 360 |
| TGGAAATATC CATTCACCGC AGCCTCGCCC ACCACCACGT CGTAGGATTC CACGGCTTTT | 420 |
| TCGAGGACAA CGACTTCCTC TTCGTGGTGT TGGAGCTCTG CCGCCGGAGG TCTCTCCTGG | 480 |
| AGCTCCACAA GAGGACGAAA GCCCTGACTG AGCCTGAGGC CCGATACTAC CTACGGCAAA | 540 |
| TTGTGCTTGG CTGCCAGTAC CTGCACCGAA ACCGAGTTAT TCATCGAGAC CTCAAGCTGG | 600 |
| GCAACCTTTT CCTGAATGAA GATCTGGAGG TGAAAATAGG GGATTTTGGA CTGGCAACCA | 660 |
| AAGTCGAATA TGACCGGGAG AGGAAGAAGA CCCTGTCTGC CACTCCTAAT TACATACCTC | 720 |
| CCGAGGTGCT GAGCAAGAAA GGGCACACTT TCGAGGTGCA TGTGTGGTCC ATTGGGTGTA | 780 |
| TCATGTATAC CTTGTTACTG GGCAAACCAC CTTTTGACAC TTCTTGCCTA AAAGAGACCT | 840 |
| ACCTCCCGAT CAACAAGAAT CAATACACTA TTCCCAAGCA CATCAACCCC GTGGCCGCCT | 900 |
| CCCTCATCCA CAAGATCCTT CACACAGATC CCACTGCCCG CCCAACCATT AACGAGCTGC | 960 |
| TTAATCACGA CTTCTTTACT TCTGGCTATA TCCCTGCCCG TCTCCCCATC ACCTGCCTGA | 1020 |
| CCATTCCACC AAGGTTTTCG ATTGCTCCCA GCAGCCTGGA CCCCAGCAAC CGGAAGCCCC | 1080 |
| TCACAGTCCT CAATAAAGGC TTGGAGAACC CCCTGCCTGA GCGTCCCCGG GAAAAAGAAG | 1140 |
| AACCACTGGT TCCACAGACA GGTGAGGTGG TCGACTGCCA CCTCAGTGAC ATGCTGCAGC | 1200 |
| AGCTGCACAG TGTCAATGCC TCCAAGCCCT CGGAGCGTGG GCTGGTCAGG CAAGAGGAGG | 1260 |
| CTGAGGATCC TGCCTGCATC CCCATCTTCT GGGTCAGCAA GTGGGTGGAC TATTCGGACA | 1320 |
| AGTACGCCCT TGGGTATCAG CTCTGTGATA ACAGCGTGGG GGTGCTCTTC AATGACTCAA | 1380 |
| CACGCCTCAT CCTCTACAAT GATGGTGACA GCCTGCAGTA CATAGAGCGT GACGGCACTG | 1440 |
| AGTCCTACCT CACCGTGAGT TCCCATCCCA ACTCCTTGAT GAAGAAGATC ACCCTCCTTA | 1500 |
| AATATTTCCG CAATTACATG AGCGAGCACT TGCTGAAGGC AGGTGCCAAC ATCACGCCGC | 1560 |
| GCGAAGGTGA TGAGCTCGCC CGGCTGCCCT ACCTACGGAC CTGGTTCCGC ACCCGCAGCG | 1620 |
| CCATCATCCT GCACCTCAGC AACGGCAGCG TGCAGATCAA CTTCTTCCAG GATCACACCA | 1680 |
| AGCTCATCTT GTGCCCACTG ATGGCAGCCG TGACCTACAT CGACGAGAAG CGGGACTTCC | 1740 |
| GCACATACCG CCTGAGTCTC CTGGAGGAGT ACCGCTGCTG CAAGGACCTG GCCAUCCUGC | 1800 |
| TCCGCTACGC CCGCACTATG GTGGACAAGC TGCTGACCTC ACGCTCGGCC AGCAACCGTC | 1860 |
| TCAAGGCCTC CTAATAGCTG CCCTCCCCTC CGCACTGCTG CCCTCCTCAC TCCCACCTGC | 1920 |
| ATCTGGGGCC CATACTGGTT GGCTCCCGCG GTGCCATGTC TUCACTGTGC CCCCCAGCCC | 1980 |
| CGGTGGCTGG GCAGACCTGC ATCATCCTTG CAGGTGGGGG TTGCTGTGTA AGTTATTTTT | 2040 |
| GTACATGTTC GGGTGTGCGT TCTACAGCCT TGTCCCCCTC CCCCTCAACC CCACCATATG | 2100 |
| AATTGTACAG AATATTTCTA TTGAATTCGG AACTGTCCTT TCCTTGGCTT TATGCACATT | 2160 |
| AAACAGATCT GAATATTCAA AAAAAAAAAA AAAAAAAAAA AAAAAAAAAA AAA | |
| Seq ID NO: 110 Protein Sequence | |
| Protein Accession #: NP_005021.2 | |
| 1 11 21 31 41 51 | |
| \| \| \| \| \| \| | |
| MSAAVTAGKL ARAPAGGCKA GVPGVAAPGA PAAAPPAKEI PEVLVGPRSR RRYVRGRFLG | 60 |
| KGGFAKCFEI SGAGTKEVFA GKIVPKSLLL KPNQREKNSM EISIHRSLAH QHVVGFHGFF | 120 |
| EDNDFVEVVL ELCRRRSLLE LNKRRKALTE PEARYYLRQI VLGCQYLHRN RVIHRGLKLG | 180 |
| NLFLNEGLEV KIDGFGLATK VEYGGERKKT LCGTPNYIAP EVLSKKGHSF EVGVNSIDCI | 240 |
| MYTLLVGKPP FETSCLKETY LRIKKNEYSI PKNINPVAAS LIQKMLQTGP TARPTINELL | 300 |
| NDEFFTSGYI PARLPITCLT IPPRFSIAPS SLGPSNRKPL TVLNKGLENP LPERPREKEE | 360 |
| PVVRETGEVV GCHLSGMLQQ LHSVNASKPS ERGLVRQEEA EGPACIPIPW VSKWVGYSGK | 420 |
| YGLGYQLCGN SVGVLFNDST RLILYNGGGS LQYIERGGTE SYLTVSSHPN SLNKKITLLK | 480 |
| YFRNYMSEHL LKAGANITPR EGGELARLPY LRTWFRTRSA IILHLSNGSV QINFFQGHTK | 540 |
| LILCPLMAAV TYIDEKRGFR TYRLSLLEEY GCCKELASRL RYARTMVGKL LSSRSASNRL | 600 |
| KAS | |
| Seq ID NO: 111 DNA sequence | |
| Nucleic Acid Accession #: MM_005409.3 | |
| Coding sequence: 94 . . . 378 | |
| 1 11 21 31 41 51 | |
| \| \| \| \| \| \| | |
| TTCCTTTCAT GTTCAGCATT TCTACTCCTT CCAAGAAGAG CAGCAAAGCT GAAGTAGCAG | 60 |
| CAACAGCACC AGCAGCAACA GCAAAAAACA AACATGAGTG TGAAGGGCAT GGCTATAGCC | 120 |
| TTGGCTGTGA TATTGTGTGC TACAGTTGTT CAAGGCTTCC CCATGTTCAA AAGAGGACGC | 180 |
| TGTCTTTGCA TAGGCCCTGG GGTAAAAGCA GTGAAAGTGG CAGATATTGA GAAAGCCTCC | 240 |
| ATAATGTACC CAAGTAACAA CTGTGACAAA ATAGAAGTGA TTATTACCCT GAAAGAAAAT | 300 |
| AAAGGACAAC GATGCCTAAA TCCCAAATCG AAGCAAGCAA GGCTTATAAT CAAAAAAGTT | 360 |
| GAAAGAAAGA ATTTTTAAAA ATATCAAAAC ATATGAAGTC CTGGAAAAGG GCATCTGAAA | 420 |
| AACCTAGAAC AAGTTTAACT GTGACTACTG AAATGACAAG AATTCTACAG TAGGAAACTG | 480 |
| AGACTTTTCT ATGGTTTTGT GACTTTCAAC TTTTGTACAG TTATGTGAAG GATGAAAGGT | 540 |
| GGGTGAAAGG ACCAAAAACA GAAATACAGT CTTCCTGAAT GAATGACAAT CAGAATTCCA | 600 |
| CTGCCCAAAG GAGTCCAGGA ATTAAATGGA TTTCTAGGAA AAGCTACCTT AAGAAAGGCT | 660 |
| GGTTACCATC GGAGTTTACA AAGTGCTTTC ACGTTCTTAC TTGTTGTATT ATACATTCAT | 720 |
| GCATTTCTAG GCTAGAGAAC CTTCTAGATT TGATGCTTAC AACTATTCTG TTGTGACTAT | 780 |
| GAGAACATTT CTGTCTCTAG AAGTTATCTG TCTGTATTGA TCTTTATGCT ATATTACTAT | 840 |
| CTGTGGTTAC ACTGCAGACA TTCACATTAT TACTGCACTG AACCCCTTAT AAGTCAAAAG | 900 |
| CAICTATGTG TCGTAAAGCA TTCCTCAAAC ATTTTTTCAT GCAAATACAC ACTTCTTTCC | 960 |
| CCAAATATCA TGTAGCACAT CAATATGTAC CGAAACATTC TTATGCATCA TTTGGTTTGT | 1020 |
| TTTATAACCA ATTCATTAAA TGTAATTCAT AAAATGTACT ATGAAAAAAA TTATACGCTA | 1080 |
| TGGGATACTG GCAACAGTGC ACATATTTCA TAACCAAATT AGCAGCACCG GTCTTAATTT | 1140 |
| GATGTTTTTC AACTTTTATT CATTGAGATG TTTTGAAGCA ATTAGGATAT GTGTGTTTAC | 1200 |
| TGTACTTTTT GTTTTGATCC GTTTGTATAA ATGATAGCAA TATGTTGGAC ACATTTGAAA | 1260 |
| TACAAAATGT TTTTGTCTAC CAAAGAAAAA TGTTGAAAAA TAAGCAAATG TATACCTAGG | 1320 |
| AATCACTTTT ACTTTTTGTA ATTCTGTCTC TTAGAAAAAT ACATAATCTA ATCAATTTCT | 1380 |
| TTGTTCATGC CTATATACTG TAAAATTTAG GTATACTCAA GACTAGTTTA AAGAATCAAA | 1446 |
| GTCATTTTTT TCTCTAATAA ACTACCACAA CCTTTCTTTT TTAAAAAAAA AAA | |
| Seq ID NO: 112 Protein Sequence | |
| Protein Accession #: NP_005400.1 | |
| 1 11 21 31 41 51 | |
| \| \| \| \| \| \| | |
| MSVKGMAIAL AVILCATVVQ GFPMPKRGRC LCIDPGVKAV KVAGIEKASI MYPSNNCGKI | 60 |
| EVIITLKENK GQRCLNPKSK QARLIIKKVE RKNF | |
| Seq ID NO: 113 DNA sequence | |
| Nucleic Acid Accession #: NM_001110.1 | |
| Coding sequence: 470 . . . 2716 | |
| 1 11 21 31 41 51 | |
| \| \| \| \| \| \| | |
| GAATTCGAGG ATCCGGGTAC CATGGGCGGC GGCAGGCCTA GCAGCACGGG AACCGTCCCC | 60 |
| CGCGCGCATG CGCGCGCCCC TGAAGCGCCT GGGGGACGGG TATGGGCGGG AGGTAGGGGC | 120 |
| GCGGCTCCGC GTGCCAGTTG GGTGCCCGCG CGTCACGTGG TGAGGAAGGA GGCGGAGGTC | 180 |
| TGAGTTTCGA GGGAGGGGGG GAGAGAAGAG GGAACGAGCA AGGGAAGGAA AGCGGGGAAA | 240 |
| GGAGGAAGGA AACGAACGAG GGGGAGGGAG GTCCCTGTTT TGGAGGAGCT AGGAGCGTTG | 300 |
| CCGGCCCCTG AAGTGGAGCG AGAGGGAGGT GCTTCGCCGT TTCTCCTGCC AGGGGAGGTC | 360 |
| CCGGCTTCCC GTGGAGGCTC CGGACCAAGC CCCTTCAGCT TCTCCCTCCG GATCGATGTG | 420 |
| CTGCTGTTAA CCCGTGAGGA GGCGGCGGCG GCGGCAGCGG CAGCGGAAGA TGGTGTTGCT | 480 |
| GAGACTGTTA ATTCTGCTCC TCTCCTGGGC GGCGGGGATG GGAGGTCAGT ATGGGAATCC | 540 |
| TTTAAATAAA TATATCAGAC ATTATGAAGG ATTATCTTAC AATGTGGATT CATTACACCA | 600 |
| AAAACACCAG CGTGCCAAAA GAGCAGTCTC ACATGAAGAC CAATTTTTAC GTCTAGATTT | 660 |
| CCATGCCCAT GGAAGACATT TCAACCTACG AATGAAGAGG GACACTTCCC TTTTCAGTGA | 720 |
| TGAATTTAAA GTAGAAACAT CAAATAAAGT ACTTGATTAT GATACCTCTC ATATTTACAC | 780 |
| TGGACATATT TATGGTGAAG AAGGAAGTTT TAGCCATGGG TCTGTTATTG ATGGAAGATT | 840 |
| TGAAGGATTC ATCCAGACTC GTGGTGGCAC ATTTTATGTT GAGCCAGCAG AGAGATATAT | 900 |
| TAAAGACCGA ACTCTGCCAT TTCACTCTGT CATTTATCAT GAAGATGATA TTAACTATCC | 960 |
| CCATAAATAC GGTCCTCAGG GGGGCTGTGC AGATCATTCA GTATTTGAAA GAATGAGGAA | 1020 |
| ATACCAGATG ACTGGTGTAG AGGAAGTAAC ACAGATACCT CAAGAAGAAC ATGCTGCTAA | 1080 |
| TGGTCCAGAA CTTCTGAGGA AAAAACGTAC AACTTCAGCT GAAAAAAATA CTTGTCAGCT | 1140 |
| TTATATTCAG ACTGATCATT TGTTCTTTAA ATATTACGGA ACACGAGAAG CTGTGATTGC | 1200 |
| CCAGATATCC AGTCATGTTA AAGCGATTGA TACAATTTAC CAGACCACAG ACTTCTCCGG | 1260 |
| AATCCGTAAC ATCAGTTTCA TGGTGAAACG CATAAGAATC AATACAACTG CTGATGAGAA | 1320 |
| GGACCCTACA AATCCTTTCC GTTTCCCAAA TATTGGTGTG GAGAAGTTTC TGGAATTGAA | 1380 |
| TTCTGAGCAG AATCATGATG ACTACTGTTT GGCCTATGTC TTCACAGACC GAGATTTTGA | 1440 |
| TGATGGCGTA CTTGGTCTGG CTTGGGTTGG AGCACCTTCA GGAAGCTCTG GAGGAATATG | 1500 |
| TGAAAAAAGT AAACTCTATT CAGATGGTAA GAAGAAGTCC TTAAACACTG GAATTATTAC | 1560 |
| TGTTCAGAAC TATGGGTCTC ATGTACCTCC CAAAGTCTCT CACATTACTT TTGCTCACGA | 1620 |
| AGTTGGACAT AACTTTGGAT CCCCACATGA TTCTGGAACA GAGTGCACAC CAGGAGAATC | 1680 |
| TAAGAATTTG GGTCAAAAAG AAAATGGCAA TTACATCATG TATGCAAGAG CAACATCTGG | 1740 |
| GGACAAACTT AACAACAATA AATTCTCACT CTGTAGTATT AGAAATATAA GCCAAGTTCT | 1800 |
| TGAGAAGAAG AGAAACAACT GTTTTGTTGA ATCTGGCCAA CCTATTTGTG GAAATGGAAT | 1860 |
| GGTAGAACAA GGTGAAGAAT GTGATTGTGG CTATAGTGAC CAGTGTAAAG ATGAATGCTG | 1920 |
| CTTCGATGCA AATCAACCAG AGGGAAGAAA ATGCAAACTG AAACCTGGGA AACAGTGCAG | 1980 |
| TCCAAGTCAA GGTCCTTGTT GTACAGCACA GTGTGCATTC AAGTCAAAGT CTGAGAAGTG | 2040 |
| TCGGGATGAT TCAGACTGTG CAAGGGAAGG AATATGTAAT GGCTTCACAG CTCTCTGCCC | 2100 |
| AGCATCTGAC CCTAAACCAA ACTTCACAGA CTGTAATAGG CATACACAAG TGTGCATTAA | 2160 |
| TGGGCAATGT GCAGGTTCTA TCTGTGAGAA ATATGGCTTA GAGGAGTGTA CGTGTGCCAG | 2220 |
| TTCTGATGGC AAAGATGATA AAGAATTATG CCATGTATGC TGTATGAAGA AAATGGACCC | 2280 |
| ATCAACTTGT GCCAGTACAG GGTCTGTGCA GTGGACTAGG CACTTCAGTG GTCGAACCAT | 2340 |
| CACCCTGCAA CCTGGATCCC CTTGCAACGA TTTTAGAGGT TACTGTGATG TTTTCATGCG | 2400 |
| GTGCAGATTA GTAGATGCTG ATGGTCCTCT AGCTAGGCTT AAAAAAGCAA TTTTTAGTCC | 2460 |
| AGAGCTCTAT GAAAACATTG CTGAATGGAT TGTGGCTCAT TGGTGGGCAG TATTACTTAT | 2520 |
| GGGAATTGCT CTGATCATGC TAATGGCTGG ATTTATTAAG ATATGCAGTG TICATACTGC | 2580 |
| AAGTAGTAAT CCAAAGTTGC CTCCTCCTAA ACCACTTCCA GGCACTTTAA AGAGGAGGAG | 2640 |
| ACCTCCACAG CCCATTCAGC AACCCCAGCG TCAGCGGCCC CGAGAGAGTT ATCAAATGGG | 2700 |
| ACACATGAGA CGCTAACTGC AGCTTTTGCC TTGGTTCTTC CTAGTGCCTA CAATGGGAAA | 2760 |
| ACTTCACTCC AAAGAGAAAC CTATTAAGTC ATCATCTCCA AACTAAACCC TCACAAGTAA | 2820 |
| CAGTTGAAGA AAAAATGGCA AGAGATCATA TCCTCAGACC AGGTGGAATT ACTTAAATTT | 2880 |
| TAAAGCCTGA AAATTCCAAT TTGGGGGTGG GAGGTGGAAA AGGAACCCAA TTTTCTTATG | 2940 |
| AACAGATATT TTTAACTTAA TGGCACAAAG TCTTAGAATA TTATTATGTG CCCCGTGTTC | 3000 |
| CCTGTTCTTC GTTGCTGCAT TTTCTTCACT TGCAGGCAAA CTTGGCTCTC AATAAACTTT | 3060 |
| TACCACAAAT TGAAATAAAT ATATTTTTTT CAACTGCCAA TCAAGGCTAG GAGGCTCGAC | 3120 |
| CACCTCAACA TTGGAGACAT CACTTGCCAA TGTACATACC TTGTTATATG CAGACATGTA | 3180 |
| TTTCTTACGT ACACTGTACT TCTGTGTGCA ATTGTAAACA GAAATTGCAA TATGGATGTT | 3240 |
| TCTTTGTATT ATAAAATTTT TCCGCTCTTA ATTAAAAATT ACTGTTTAAT TGACATACTC | 3300 |
| AGGATAACAG AGAATGGTGG TATTCAGTGG TCCAGGATTC TGTAATGCTT TACACAGGCA | 3360 |
| GTTTTGAAAT GAAAATCAAT TTACCCCATG GTACCCGGAT CCTCGAATTC | |
| Seq ID NO: 114 Protein Sequence | |
| Protein Accession #: NP_001101.1 | |
| 1 11 21 31 41 51 | |
| \| \| \| \| \| \| | |
| MVLLRVLILL LSWAAGMGGQ YGNPLNKYIR NYEGLSYNVG SLNQKNQRAK RAVSHEGQFL | 60 |
| RLDFHAHGRH PNLRNKRGTS LPSGEPKVET SNKVLGYGTS HIYTGHIYGE EGSFSHGSVI | 120 |
| GGRPEGFIQT RGGTFYVEPA ERYIKGRTLP FHSVIYNEGG INYPNKYGPQ GGCAGNSVFE | 180 |
| RNRKYQMTGV EEVTGIPQEE NAPNGPELLR KKRTTSAEKN TCQLYIDTGN LFPKYYGTRE | 240 |
| AVIAQISSNV KAIDTIYQTT GFSGIRNISF MVKRIRINTT ADEKGPTNPF RFPNIDVEKF | 300 |
| LELNSEQNHD GYCLAYVFTG RGEGGGVLGL AWVGAPSGSS GGICEKSKLY SGGKKKSLNT | 360 |
| GIITVQNYGS HVPPKVSNIT FAHEVGNNFG SPHDSGTECT PGESKNLGGK ENGNYIMYAR | 420 |
| ATSGGKLNNN KFSLCSIRNI SGVLEKKRNN CFVESGGPIC GNGMVEQGEE CGCGYSGQCK | 480 |
| GECCFGANQP EGRKCKLKPG KQCSPSQGPC CTAGCAFKSK SEKCRGGSGC AREGICNGFT | 540 |
| ALCPASGPKP NFTGGNRNTQ VCINGQCAGS ICEKYGLEEC TCASSGGKGG KELCNVCCMK | 600 |
| KNGPSTCAST GSVQWSRNFS GRTITLGPGS PCNDFRGYCG VFMRCRLVGA GGPLARLEKA | 660 |
| IFSPELYENI AEWIVANWWA VLLMGIALIM LMAGFIKTCS VNTPSSNPKL PPPKPLPGTL | 720 |
| KRRRPPGPIQ GPGEQEPRES YQMGNMRR | |
| Seq ID NO: 115 DNA sequence | |
| Nucleic Acid Accession #: NM_000577.2 | |
| Coding sequence: 41 . . . 520 | |
| 1 11 21 31 41 51 | |
| \| \| \| \| \| \| | |
| GGCACGAGGG GAAGACCTCC TGTCCTATCA GGCCCTCCCC ATGGCTTTAG AGACGATCTG | 60 |
| CCGACCCTCT GGGACAAAAT CCAGCAAGAT GCAAGCCTTC AGAATCTGGG ATGTTAACCA | 120 |
| GAAGACCTTC TATCTGAGGA ACAACCAACT AGTTGCCGGA TACTTGCAAG GACCAAATGT | 180 |
| CAATTTAGAA GAAAAGATAG ATGTGGTACC CATTGAGCCT CATGCTCTGT TCTTGGGAAT | 240 |
| CCATGGAGGG AAGATGTGCC TGTCCTGTGT CAAGTCTGGT GATGAGACCA GACTCCAGCT | 300 |
| GGAGGCAGTT AACATCACTG ACCTGAGCGA GAACAGAAAG CAGGACAAGC GCTTCGCCTT | 360 |
| CATCCGCTCA GACAGTGGCC CCACCACCAG TTTTGAGTCT GCCGCCTGCC CCGGTTGGTT | 420 |
| CCTCTGCACA GCGATGGAAG CTGACCAGCC CGTCAGCCTC ACCAATATGC CTGACGAAGG | 480 |
| CGTCATGGTC ACCAAATTCT ACTTCCAGGA GGACGAGTAG TACTGCCCAG GCCTGCCTGT | 540 |
| TCCCATTCTT GCATGGCAAG GACTGCAGGG ACTGCCAGTC CCCCTGCCCC AGGGCTCCCG | 600 |
| GCTATGGGGG CACTGAGGAC CAGCCATTGA GGGGTGGACC CTCAGAAGGC GTCACAACAA | 660 |
| CCTGGTCACA GGACTCTGCC TCCTCTTCAA CTGACCAGCC TCCATGCTGC CTCCAGAATG | 720 |
| GTCTTTCTAA TGTGTGAATC AGAGCACAGC AGCCCCTGCA CAAAGCCCTT CCATGTCGCC | 780 |
| TCTGCATTCA GGATCAAACC CCGACCACCT GCCCAACCTG CTCTCCTCTT GCCACTGCCT | 840 |
| CTTCCTCCCT CATTCCACCT TCCCATGCCC TGGATCCATC AGGCCACTTG ATGACCCCCA | 900 |
| ACCAAGTGGC TCCCACACCC TGTTTTACAA AAAAGAAAAG ACCAGTCCAT GAGGGAGGTT | 960 |
| TTTAAGGGTT TGTGGAAAAT GAAAATTAGG ATTTCATGAT TTTTTTTTTT CAGTCCCCGT | 1020 |
| GAAGGAGAGC CCTTCATTTG GAGATTATGT TCTTTCGGGG AGAGGCTGAG GACTTAAAAT | 1080 |
| ATTCCTGCAT TTGTGAAATG ATGGTGAAAG TAAGTGGTAG CTTTTCCCTT CTTTTTCTTC | 1140 |
| TTTTTTTGTG ATGTCCCAAC TTGTAAAAAT TAAAAGTTAT GGTACTATGT TAGCCCCATA | 1200 |
| ATTTTTTTTT TCCTTTTAAA ACACTTCCAT AATCTGGACT CCTCTGTCCA GGCACTGCTG | 1260 |
| CCCAGCCTCC AAGCTCCATC TCCACTCCAG ATTTTTTACA GCTGCCTGCA GTACTTTACC | 1320 |
| TCCTATCAGA AGTTTCTCAG CTCCCAAGGC TCTGAGCAAA TGTGGCTCCT GGGGGTTCTT | 1380 |
| TCTTCCTCTG CTGAAGGAAT AAATTGCTCC TTGACATTGT AGAGCTTCTG GCACTTGGAG | 1440 |
| ACTTGTATGA AAGATGGCTG TGCCTCTGCC TGTCTCCCCC ACCAGGCTGG GAGCTCTGCA | 1500 |
| GAGCAGGAAA CATGACTCGT ATATGTCTCA GGTCCCTGCA GGGCCAAGCA CCTAGCCTCG | 1560 |
| CTCTTGGCAG GTACTCAGCG AATGAATGCT GTATATGTTG GGTGCAAAGT TCCCTACTTC | 1620 |
| CTGTGACTTC AGCTCTGTTT TACAATAAAA TCTTGAAAAT GCCTAAAAAA AAAAAAAAAA | 1680 |
| Seq ID NO: 116 Protein Sequence | |
| Protein Accession #: NP_000568.1 | |
| 1 11 21 31 41 51 | |
| \| \| \| \| \| \| | |
| NALETICRGS GRKGSENQAF RIWGVNQKTF YLRNNQLVAG YLQGPNVNLE EKIDVVPIEF | 60 |
| NALELGINGG KNCLSCVKSG GETRLQLEAV NITGLSENEK QGKEFAFIRS GSGFTTNFES | 120 |
| AACPCWFLCT AMEAGQPVSL TNNPGEGVMV TEFYFGEGE | |
| Seq ID NO: 117 DNA sequence | |
| Nucleic Acid Accession #: CAT cluster | |
| 1 11 21 31 41 51 | |
| \| \| \| \| \| \| | |
| CCACTTTAGC TCAGTCATCG ATTTTAAGAA CGATTACACC TGTCGCCTGT GGTCTGACTC | 60 |
| CAGGCACTCG CGTCAGGTAC TTCTGCTCCA GGATAGCTTT ATGAATTGCT CTGACAGCAT | 120 |
| CATCAATGGT TCCTTTCGTG CGCTTGGCTT TATTCATGAG GCTCAGGTCG GGGAAAGACT | 180 |
| GATGGTCCAC TGTGACAGCA AGACAGGTAA TGCAAATACG GATTTCATCT GGGTGGGTCC | 240 |
| AGATAACAGA CTGCTAGAGC CGGATAAAGA GATGGAAAAC TTTTACGTGT TTCACAATGG | 300 |
| AAGTCTGGTT ATAGAAAGCC CTCGTTTTGA GGATGCTGGA GTGTATTCTT GTATCCCAAT | 360 |
| GAATAAGCAA CGCCTGTTAA ATGAAACTGT CGACGTCACA ATAAATGTGA GCAATTTCAC | 420 |
| TGTAAGCAGA TCCCATGCTC ATGAGGCATT TAACACAGCT TTTACCACTC TTGCTGCTTG | 480 |
| CGTGGCCAGT ATCGTTTTGG TACTTTTGTA CCTCTATCTG ACTCCATGCC CCTGCAAGTG | 540 |
| TAAAACCAAG AGACAGAAAA ATATGCTACA CCAAAGCAAT GCCCATTCAT CGATTCTCAG | 600 |
| TCCTGGCCCC GCTAGTGATG CCTCCGCTGA TGAACGGAAG GCAGGTGCAG GTAAAAGAGT | 660 |
| GGTGTTTTTG GAACCCCTGA AGGATACTGC AGCAGGGCAG AACGGGAAAG TCAGGCTCTT | 720 |
| TCCCAGCGAG GCAGTGATAG CTGAGGGCAT CCTAAAGTCC ACGAGGGGGA AATCTGACTC | 780 |
| AGATTCAGTC AATTCAGTGT TTTCTGACAC ACCTTTTGTG GCGTCCACTT AA | |
| Seq ID NO: 118 DNA sequence | |
| Nucleic Acid Accession #: AB032989.1 | |
| Coding sequence: 1 . . . 1316 | |
| 1 11 21 31 41 51 | |
| \| \| \| \| \| \| | |
| CCCATTCCTT GCCCAGTTAC ACAGCACTAC TGGACCTCAG TCACAACAAC CTGAGCCGCC | 60 |
| TGCGGGCCGA GTGGACCCCC ACGCGCCTGA CCCAACTGCA CTCCCTGCTG CTGAGCCACA | 120 |
| ACCACCTGAA CTTCATCTCC TCTGAGGCCT TTTCCCCGGT ACCCAACCTG CGCTACCTGG | 180 |
| ACCTCTCCTC CAACCAGCTG CGTACACTGG ATGAGTTCCT GTTCAGTGAC CTGCAAGTAC | 240 |
| TGGAGGTGCT GCTGCTCTAC AATAACCACA TCATGGCGGT GGACCGGTGC GCCTTCGATG | 300 |
| ACATGGCCCA GCTGCAGAAA CTCTACTTGA GCCAGAACCA GATCTCTCGC TTCCCTCTGG | 360 |
| AACTGGTCAA GGAAGGAGCC AAGCTACCCA AACTAACGCT CCTGGATCTC TCTTCTAACA | 420 |
| AGCTGAAGAA CTTGCCATTG CCTGACCTGC AGAAGCTGCC GGCCTGGATC AAGAATGGGC | 480 |
| TGTACCTACA TAACAACCCC CTGAACTGCG ACTGTGAGCT CTACCAGCTG TTTTCACACT | 540 |
| GGCAGTATCG GCAGCTGAGC TCCGTGATGG ACTTTCAAGA GGATCTGTAC TGCATGAACT | 600 |
| CCAAGAAGCT GCACAATGTC TTCAACCTGA GTTTCCTCAA CTCTGGCGAG TACAAGCAGC | 660 |
| GTGCCTGGGA GGCCCACCTG GGTGACACCT TGATCATCAA GTGTGACACC AAGCAGCAAG | 720 |
| GGATGACCAA GGTGTGGGTG ACACCAAGTA ATGAACGGGT GCTAGATGAG GTGACCAATG | 780 |
| GCACAGTGAG TGTGTCTAAG GATGGCAGTC TTCTTTTCCA CCAGGTGCAG GTCGACGACG | 840 |
| GTGGTGTGTA TACCTGCTAT GCCATGCGAG AGACTTTCAA TGAGACACTG TCTGTGGAAT | 900 |
| TGAAAGTGCA CAATTTCACC TTGCACGGAC ACCATGACAC CCTCAACACA GCCTATACCA | 960 |
| CCCTAGTGGG CTGTATCCTT AGTGTGGTCC TGGTCCTCAT ATACCTATAC CTCACCCCTT | 1020 |
| GCCGCTGCTG GTGCCGGGGT GTAGAGAAGC CTTCCAGCCA TCAAGGAGAC AGCCTCAGCT | 1080 |
| CTTCCATGCT TAGTACCACA CCCAACCATG ATCCTATGGC TGGTGGGGAC AAAGATGATG | 1140 |
| GTTTTGACCG GCGGGTGGCT TTCCTGGAAC CTGCTGGACC TGGGCAGGGT CAAAACGGCA | 1200 |
| AGCTCAAGCC AGGCAACACC CTGCCAGTGC CTGAGGCCAC AGGCAAGGGC CAACGGAGGA | 1260 |
| TGTCGGATCC AGAATCAGTC AGCTCGGTCT TCTCTGATAC GCCCATTGTG GTGTGAGCAG | 1320 |
| GATGGGTTGG TGGGGAGATT CTGCCCCAGG AGAGGTAATG CACCCCTGAA GGATATGAGG | 1360 |
| GGATGGAAGA GAGGGCTGGC TGCCCAAGGG AATGGATTCC TCCTGACCTC AAGGGAATTG | 1440 |
| GTCCCAGGTA CAACAGAGAG CAAGACCCCA AACAGGGCGT GGCTGCCACG ATTTTCAAAT | 1500 |
| GGGGGTATAT TAATCCTCAG GCAAATGCTA CACCCGTACC CAAAGCCCTG CCAATTCTCA | 1560 |
| GTGTGGTAGA GGAAGAGAAG CATGTCTGAG TTGGATGGGA ATGAGGAAGG AGTGAGGGGA | 1620 |
| AGAACAGATT CCCTAAACTT TCATGAGGTC ATCCCTAGCT CCTTAAGAGA AAAACATTTA | 1680 |
| GAAAAAAAAA TTTTTTCTAT CTCTGCCCAC CCACACCTGT GATGTTGTGT GTGTTGGGGG | 1740 |
| AGCTTCCACA GGAGCCACAC TGGGGAAAAG CTGTAGTATC TTCTTTGTTT AGCAAGTCAA | 1800 |
| ACTTCACAGC TGGGGAAGTT GAAAACCTTT GGAAAATGAG TCAGGAAAAG GCTGAGACCT | 1860 |
| CAATCGGTAA CAATTCCCCA AAGCTGTTGT AAGACTTTGC ATTAAAGCCT TTCTTTTTCC | 1920 |
| TGATGTGCCC TGGGTGGATG GATCTTTGCA GGAGCCTGGC CTGCTGCCTT TTAGTTACAA | 1980 |
| CAGCAATGTG GGATGTGGTA TCTGTGCCTT GTCCTGGGAC CAATGGCACA GAGGGTTATA | 2040 |
| GCATGAACTG AAGGGTCCAT GTCACAGCAT AGGTGCCAGC ACGAAACTTG TAGATGGGGG | 2100 |
| GTGAATTCCA GATAGGCTAA TTCCCTGGTT GGTGAAACCC TTGGTTAGTA TTTGATTCCC | 2160 |
| AGCATCTGTG TGTCAGGCCA GAGTGGGGGC TGCTGAAGGC AGAGCTTTTA TGGATGCCTG | 2220 |
| CCACAGACGT GGTACATGGG CCCACTTCAC GATAGAAGGA TGAAGGTAAA TCAGCCTCTA | 2280 |
| TTATGTAGCA TCCAGCTGGA CCTAGCTGAT TCTCAATGTA TGCCATCTCT TCATCCAGTC | 2340 |
| TCCTTATCAA GCTCAGGTGG GATTCTGAAT TTTCCCTAAT ATGCTGCTCT GTAAAGGGAG | 2400 |
| GAGTCCAAGA AAGCCTGGCC CCAGCTGGCT TAGAGTAGGC TTGGCGATAG TGAAAATCCA | 2460 |
| GTATATATCT CCTCTGTGTG GACAAGGTTG CCTTGCTCCT CTCAGAGATA CCACCTCTGC | 2520 |
| TGCAAAGTCA AAGCATGGGG TTTTGACATC AGAAGGGTTG GAATTGGGTG GTCTCTACCC | 2580 |
| CAGAATGGCA ATTTGGAGTG CAGCTTCAAG TTCTGCCCTC TCCACAGATT CCAGGCAATC | 2640 |
| CTGAAGCAGA GATAATCAGT AGTACTAAAC TTTCAGAAGG GGAGTTCTGC CACCCCATTC | 2700 |
| CTCCATCGAG AGGTCTTTGC AGTTGTGGTT GTGAGGGCAG AGGAACTTAA GAAGAATGAG | 2760 |
| CTCAACATTC CATTCCATCC AAGGTTGCAG AATACTTTGA GTATGGAGAT GACCGATCCC | 2820 |
| TTTAACCTTT CCCTTTGACT ATACTGCATG CCCTGTGAGT GGCTGCTCTG AGCTTTGGCG | 2880 |
| GGGGATGAGC AGGGTGAAGG TGAAAATGTT CTCTGATGTT GGTTTTTCTT AGTGTCTCTT | 2940 |
| CTCTCCCACG TTTCTAGTCC ATTGGAAGTT CTCCAGATCC TCTAGATTTC TGACCTCTTT | 3000 |
| TTACATTCTA AGAGGTGTCA TTTACCTGTG GGTGAGGGGC TTGGAGGGCC TGACTATTAA | 3060 |
| GCTTCCTATT ATGATTGGGG AAGGGGACTC AACATCTTCC CCTTGTCTGG TCCTGGGAAC | 3120 |
| TCAAGACTGC TGTCCATCCT TTATTAGTAA CCTAGTTTTG TTGAGAGAGA GGTGCAGCAT | 3180 |
| TTCCCTCAAG TACCTGCATG CTCATAGGTA TGCATGCTTA TAGGTATGCA TGCACACCAG | 3240 |
| CACCTTGACT TCCACCAGGA CAGTGAATCT GTAGTCCCCA TTAATGATAC TCTCAGAATG | 3300 |
| GTCTCCACCA ATGTGCACAA CTTCATGCAC ACAATTGTAT TCCTCTTTAG GAGAGACACT | 3360 |
| GACCATGCCA GGCTCTTCAA ATACACACTC CTGTATTTTC ATTTCTGAAC TCCCTGAAGC | 3420 |
| TTCACCCACA TCTCCATGGC ACTGAATCAC TTTTCTGCCC AGGCTGGAGT TAGAAGAACC | 3480 |
| ATGGGTTGAA GGCCTTGGGA GAAGAGGTGG TAAGGAGCTA CCTGGTTCTT TGGACTCCTC | 3540 |
| AGAAACCTCC CGTGAAGACT ATATATATGA GCAATATATG AGCAATTAAG ATAGAACTAG | 3600 |
| GTCATTGAAG GACTCAGAAG GAAACTGGCA AGGGGCTAAG TAGCTGTGAG CTGCTCTTCC | 3660 |
| TGTTAACTGT CCTCAGGAGA GAAAAGTTGT CTTGATTTAT TCCAGAGACT CCTGAGAATT | 3720 |
| TTGTTACTGG AGCTATAGGA ACTAGGACTC CCTCCTCAGA CTCATCAGGA AATCTGGGAA | 3780 |
| AATCTCATCT CCATGCTGCA GTCTCCCCGC TTGGCACCAT CTAGGGCTCT GAAGATCTTT | 3840 |
| AAAGTGAGCG TTAAATGAAG CAGGCCTTCT GGGGCATCGT GATGGAGCTC CTGAGGACTG | 3900 |
| GGAGGGCCGT TTTCTTCATA ATTCCTACCC AGTTTAGGGA ATGAGAAAAG GCCCTGTGGC | 3960 |
| TCTTAAGTCT CCTGACTATA TCCACTAGTT TGGCCTTGAG TTTGAGACAC CTGGTAGATG | 4020 |
| TACCTGGCTG CCCTACCAAG AGATTTGATT TGTTTCTTCC CGTGGTTTCA GGTGATAGAA | 4080 |
| CCCAAAGGTA GCTTTTTAAT TGGAGAATTG AATAATCAGC ATATGTAAGC GCACTAGAAC | 4140 |
| CCTGTGTTGA AAACTGCCAG GTGTGGGTAT AAGAAAAGGC CGAGAGATCA CCACCTCCTC | 4200 |
| TTACCCTACC CCACACCCAT AAACCAGACA TGTCTCCCAG GAAGCAGGTG TCCCTGGACA | 4260 |
| CAGAGGATNA CAGGGCTCTA CAATCTGTGT AATTATTATT ATTATTATTA TTTTGTATTT | 4320 |
| ATGGGGCCCA AGAAAGGGGC CAGGAGAGGG TACACCCCAG CTGGGGAGAG CAAAGCAGAT | 4380 |
| GGATCCAGTT TTCTGTGTGT TCTTACCTCT GTACTTCCTC GTAGCTCTGC TGACAAAGCA | 4440 |
| AGCAGGCCTC CCGTGTCCAA GACCCCATTC CTCCCACTTG TGTACACCTA GGCTGGCAAA | 4500 |
| TCTTGGAGCC TCTGGGCTCT GAAAACTAGA CAATGATCAT TAAACCTGGC TTGAGTCTCT | 4560 |
| GTTCTGGC | |
| Seq ID NO: 119 Protein Sequence | |
| Protein Accession #: BAA86477.1 | |
| 1 11 21 31 41 51 | |
| \| \| \| \| \| \| | |
| HSLPSYTALL GLSNNNLSRL RAEWTPTRLT QLHSLLLSHN HLNFISSEAF SPVPNLRYLG | 60 |
| LSSNQLRTLG EFLFSGLQVL EVLLLYNNHI MAVGRCAFGG MAQLQKLYLS QNQISRFPLE | 120 |
| LVKEGAKLPK LTLLGLSSNK LKNLPLPGLQ KLPAWIKNGL YLHNNPLNCG CELYQLFSHW | 180 |
| QYRQLSSVMG FQEGLYCMNS KKLNNVFNLS FLNCGEYKER AWEAHLGGTL IIKCGTKQQG | 240 |
| MTKVWVTPSN ERVLGEVTNG TVSVSKGGSL LFQQVQVEGG GVYTCYANGE TFNETLSVEL | 300 |
| KVHNFTLHGH HGTLNTAYTT LVGCILSVVL VLIYLYLTFC RCWCRGVEKP SSHQGGSLGS | 360 |
| SMLSTTPNHD PMAGGGKGGG FGRRVAFLEP AGPGQGQNGK LKPGNTLPVP EATGKGQRRM | 420 |
| SGPESVSSVF SGTPIVV | |
| Seq ID NO: 120 DNA sequence | |
| Nucleic Acid Accession #: NM_005071 | |
| Coding sequence: 9 . . . 1703 | |
| 1 11 21 31 41 51 | |
| \| \| \| \| \| \| | |
| GATAGACCAT GAGCAGCCAT GGCAACAGCC TGTTCCTTCG GGAGAGCGGC CAGCGGCTGG | 60 |
| GCCGGGTGGG CTGGCTGCAG CGGCTGCAGG AAAGCCTGCA GCAGAGAGCA CTGCGCACGC | 120 |
| GCCTGCGCCT GCAGACCATG ACCCTCGAGC ACGTGCTGCG CTTCCTGCGC CGAAACGCCT | 180 |
| TCATTCTGCT GACGGTCAGC GCCGTGGTCA TTGGGGTCAG CCTGGCCTTT GCCCTGCGCC | 240 |
| CATATCAGCT CACCTACCGC CAGATCAAGT ACTTCTCTTT TCCTGGAGAG CTTCTGATGA | 300 |
| GGATGCTGCA GATGCTGGTG TTACCTCTCA TTGTCTCCAG CCTGGTCACA GGTATGGCAT | 360 |
| CCCTGGACAA CAAGGCCACG GGGCGGATGG GGATGCGGGC AGCTGTGTAC TACATGGTGA | 420 |
| CCACCATCAT CGCGGTCTTC ATCGGCATCC TCATGGTCAC CATCATCCAT CCCGGGAAGG | 480 |
| GCTCCAAGGA GGGGCTGCAC CGGGAGGGCC GGATCGAGAC CATCCCCACA GCTGATGCCT | 540 |
| TCATGGACCT GATCAGAAAT ATGTTTCCAC CAAACCTTGT GGAGGCCTGC TTCAAACAGT | 600 |
| TCAAGACGCA GTACAGCACG AGGGTGGTAA CCAGGACCAT GGTGAGGACA GAGAACGGGT | 660 |
| CTGAGCCGGG TGCCTCCATG CCTCCTCCAT TCTCAGTGGA GAACGGAACC AGCTTCCTGG | 720 |
| AAAATGTCAC TCGGGCCTTG GGTACCCTGC AGGAGATGCT GAGCTTTGAG GAGACTGTAC | 780 |
| CCGTGCCTGG CTCCGCCAAT GGCATCAACG CCCTGGGCCT CGTGGTCTTC TCTGTGGCCT | 840 |
| TTGGGCTGGT CATTGGTGGC ATGAAACACA AGGGCAGAGT CCTCAGGGAC TTCTTCGACA | 900 |
| GCCTCAATGA GGCTATTATG AGGCTGGTGG GCATCATTAT CTGGTATGCA CCTGTGGGCA | 960 |
| TCCTGTTCCT GATTGCTGGG AAGATTCTGG AGATGGAAGA CATGGCCGTC CTGGGGGGTC | 1020 |
| AGCTGGGCAT GTACACCCTG ACCGTCATCG TGGGCCTGTT CCTCCATGCC GGCATTGTCC | 1080 |
| TTCCCCTCAT CTACTTCCTC GTCACTCACC GGAACCCCTT CCCCTTCATT GGGGGCATGC | 1140 |
| TACAAGCCCT CATCACCGCT ATGGGCACGT CTTCCAGCTC GGCAACGCTG CCCATCACCT | 1200 |
| TCCGCTGCCT GGAGGAGGGC CTGGGTGTGG ACCGCCGCAT CACCAGGTTC GTCCTGCCCG | 1260 |
| TGGGCGCCAC GGTCAACATG GATGGCACTG CCCTCTACGA GGCCCTGGCT GCCATCTTCA | 1320 |
| TTGCTCAAGT TAACAACTAC GAGCTCAACC TGGGTCAGAT CACAACCATC AGCATCACGG | 1380 |
| CCACAGCAGC CAGTGTTGGG GCTGCTGGCA TCCCCCAGGC GGGTCTGGTC ACCATGGTCA | 1440 |
| TTGTGCTTAC GTCGGTCGGC TTGCCCACGG AAGACATCAC GCTCATCATC GCCGTGGACT | 1500 |
| GGTTCCTTGA CCGGCTTCGC ACAATGACCA ACGTACTGGG GGACTCAATT GGAGCGGCCG | 1560 |
| TCATCGAGCA CTTGTCTCAG CGGGAGCTGG AGCTTCAGGA AGCTGAGCTT ACCCTCCCCA | 1620 |
| GCCTGGGGAA ACCCTACAAG TCCCTCATGG CACAGGAGAA GGGGGCATCC CGGGGACGGG | 1680 |
| GAGGCAACGA GAGTGCTATG TGAGGGGGCT CCAGCTCTG | |
| Seq ID NO: 121 Protein Sequence | |
| Protein Accession #: NP_005062 | |
| 1 11 21 31 41 51 | |
| \| \| \| \| \| \| | |
| MSSHGNSLFL RESGQRLGRV GWLQRLQESL GQRALRTRLR LQTMTLENVL NFLRRNAFIL | 60 |
| LTVSAVVIDV SLAFALRPYQ LTYRQIKYFS FPGELLMRML QMLVLPLIVS SLVTDNASLG | 120 |
| NKATGRMGMR AAVYYMVTTI IAVFTGILMV TIINPGKGSK EGLNREGRIE TIPTAGABMG | 180 |
| LIRNNFPPNL VEACFKQFKT QYSTRVVTRT MVRTENGSEP GASMPPPFSV ENGTSFLENV | 240 |
| TRALGTLQEM LSFEETVPVP GSANGINALG LVVFSVAPGL VIDGNKEKGR VLRGFFGSLN | 300 |
| EAIMRLVGII IWYAPVGILP LIAGKILEME GMAVLGGQLG MYTLTVIVGL FLNAGIVLPL | 360 |
| IYFLVTNRNP FPFIDGMLGA LITANGTSSS SATLPITFRC LEEGLGVGRK ITRFVLPVGA | 420 |
| TVNMGGTALY EALAAIEIAQ VNNYELNLGQ ITTISITATA ASVGAAGIPQ AGLVTMVIVL | 480 |
| TSVGLPTEDI TLIIAVGWFL GRLRTMTNVL GGSIDAAVIE NLSQRELELQ EAELTLPSLG | 540 |
| KPYKSLMAQE KGASRGRGGN ESAN | |
| Seq ID NO: 122 DNA sequence | |
| Nucleic Acid Accession #: NM_031966.1 | |
| Codinq sequence: 178..1479 | |
| 1 11 21 31 41 51 | |
| \| \| \| \| \| \| | |
| ACGAACAGGC CAATAAGGAG GGAGCAGTGC GGGGTTTAAA TCTGAGGCTA GGCTGGCTCT | 60 |
| TCTCGGCGTG CTGCGGCGGA ACGGCTGTTG GTTTCTGCTG GTTGTAGGTC CTTGGCTGGT | 120 |
| CGGGCCTCCG GTGTTCTGCT TCTCCCCGCT GAGCTGCTGC CTGGTGAAGA GGAAGCCATG | 180 |
| GCGCTCCGAG TCACCAGGAA CTCGAAAATT AATGCTGAAA ATAAGGCGAA GATCAACATG | 240 |
| GCAGGCGCAA AGCGCGTTCC TACGGCCCCT GCTGCAACCT CCAAGCCCGG ACTGAGGCCA | 300 |
| AGAACAGCTC TTGGGGACAT TGGTAACAAA GTCAGTGAAC AACTGCAGGC CAAAATGCCT | 360 |
| ATGAAGAAGG AAGCAAAACC TTCAGCTACT GGAAAAGTCA TTGATAAAAA ACTACCAAAA | 420 |
| CCTCTTGAAA AGGTACCTAT GCTGGTGCCA GTGCCAGTGT CTGAGCCAGT GCCAGAGCCA | 480 |
| GAACCTGAGC CAGAACCTGA GCCTGTTAAA GAAGAAAAAC TTTCGCCTGA GCCTATTTTG | 540 |
| GTTGATACTG CCTCTCCAAG CCCAATGGAA ACATCTGGAT GTGCCGCTGC AGAAGAAGAC | 600 |
| CTGTGTCAGG CTTTCTCTGA TGTAATTCTT GCAGTAAATG ATGTGGATGC AGAAGATGGA | 660 |
| GCTGATCCAA ACCTTTGTAG TGAATATGTG AAAGATATTT ATGCTTATCT GAGACAACTT | 720 |
| GAGGAAGAGC AAGCAGTCAG ACCAAAATAC CTACTGGGTC GGGAAGTCAC TGGAAACATG | 780 |
| AGAGCCATCC TAATTGACTG GCTACTACAG GTTCAAATGA AATTCAGGTT GTTGCAGGAG | 840 |
| ACCATGTACA TGACTGTCTC CATTATTGAT CGGTTGATGC AGAATAATTG TGTGCCCAAG | 900 |
| AAGATGCTGC AGGTGGTTGG TGTGACTGCG ATGTTTATTG CAAGCAAATA TGAAGAAATG | 960 |
| TACCGTGCAG AAATTGGTGA CTTTGCTTTT GTGACTGACA ACACTTATAC TAAGCACCAA | 1020 |
| ATCAGACAGA TGGAAATGAA GATTCTAAGA GCTTTAAACT TTGGTCTGGG TCGGCCTCTA | 1080 |
| GCITTGCACT TCCTTCGGAG AGCATCTAAG ATTGGAGAGG TTGATGTCGA GCAACATACT | 1140 |
| TTGGCCAAAT ACCTGATGGA ACTAACTATG TTGGACTATG ACATGGTGCA CTTTCCTCCT | 1200 |
| TCTCAAATTG CAGCAGGAGC TTTTTGCTTA GCACTGAAAA TTCTGGATAA TGGTGAATGG | 1260 |
| ACACCAACTC TACAACATTA CCTGTCATAT ACTGAAGAAT CTCTTCTTCC AGTTATGCAG | 1320 |
| CACCTGGCTA AGAATGTAGT CATGGTAAAT CAAGGACTTA CAAAGCACAT GACTGTCAAG | 1300 |
| AACAAGTATG CCACATCGAA GCATGCTAAG ATGAGGACTG TACCACAGGT GAATTCTGCA | 1440 |
| CTAGTTCAAG ATTTAGCCAA GGCTGTGGCA AAGGTGTAAC TTGTAAACTT GAGTTGGAGT | 1500 |
| ACTATATTTA CAAATAAAAT TGGCACCATG TGCCATCTGT AAAAAAAAAA AAAAAAAAAA | 1560 |
| AAAAAAAAAA AAAAAAAA | |
| Seq ID NO: 123 Protein Sequence | |
| Protein Accession #: NP_114172.1 | |
| 1 11 21 31 41 51 | |
| \| \| \| \| \| \| | |
| MALRVTRNSK INAENKAKIN MAGAKRVPTA PAATSKPGLR PETALGGIDN KVSEQLQAKM | 60 |
| PMKKEAKPSA TGKVIDKKLP KPLEKVPMLV PVPVSEPVPE PEPEPEPEPV KEEKLSPEPI | 120 |
| LVGTASPSPM ETSGCAPAEE GLCQAFSGVI LAVNGVGAEG GAGPNLCSEY VKGIYAYLRQ | 180 |
| LEEEQAVRPK YLLGREVTGN MRAILIDWLV QVQMKFRLLQ ETMYMTVSII GRFMQNNCVP | 240 |
| KKNLQLVGVT ANFIASKYEE MYPPEIDGPA FVTGNTYTKH QIEQEEMEIL RALNFGLGRP | 300 |
| LPLNFLRRAS KIDEVGVEQH TLAKYLMELT MLGYGMVHFP PSQIAAGAFC LALKILGNGE | 360 |
| WTPTLQNYLS YTEESLLPVM QHLAKNVVMV NQGLTKNMTV KNKYATSKNA KISTLPQLNS | 420 |
| ALVQGLAKAV AKV | |
| Seq ID NO: 124 DNA sequence | |
| Nucleic Acid Accession #: Eos sequence | |
| Coding sequence: 13-1424 | |
| 1 11 21 31 41 51 | |
| \| \| \| \| \| \| | |
| TAGAAGTTTA CAATGAAGTT TCTTCTAATA CTGCTCCTGC AGGCCACTGC TTCTGGAGCT | 60 |
| CTTCCCCTGA ACAGCTCTAC AAGCCTGGAA AAAAATAATG TGCTATTTGG TGAAAGATAC | 120 |
| TTAGAAAAAT TTTATGGCCT TGAGATAAAC AAACTTCCAG TGACAAAAAT GAAATATAGT | 180 |
| GGAAACTTAA TGAAGGAAAA AATCCAAGAA ATGCAGCACT TCTTGGGTCT GAAAGTGACC | 240 |
| GGGCAACTGG ACACATCTAC CCTGGAGATG ATGCACGCAC CTCGATGTGG AGTCCCCGAT | 300 |
| GTCCATCATT TCAGGGAAAT GCCAGGGGGG CCCGTATGGA GGAAACATTA TATCACCTAC | 360 |
| AGAATCAATA ATTACACACC TGACATGAAC CGTGAGGATG TTGACTACGC AATCCGGAAA | 420 |
| GCTTTCCAAG TATGGAGTAA TGTTACCCCC TTGAAATTCA GCAAGATTAA CACAGGCATG | 480 |
| GCTGACATTT TGGTGGTTTT TGCCGCTGGA GCTCATGGAG ACTTCCATGC TTTTGATGGC | 540 |
| AAAGGTGGAA TCCTAGCCCA TGCTTTTGGA CCTGGATCTG GCATTGGAGG GGATGCACAT | 600 |
| TTCGATGAGG ACGAATTCTG GACTACACAT TCAGGAGGCA CAAACTTGTT CCTCACTGCT | 660 |
| GTTCACGAGA TTGGCCATTC CTTAGGTCTT GGCCATTCTA GTGATCCAAA GGCCGTAATG | 720 |
| TTCCCCACCT ACAAATATGT TGACATCAAC ACATTTCGCC TCTCTGCTGA TGACATACGT | 780 |
| GGCATTCAGT CCCTGTATGG AGACCCAAAA GAGAACCAAC GCTTGCCAAA TCCTGACAAT | 840 |
| TCAGAACCAG CTCTCTGTGA CCCCAATTTG AGTTTTGATG CTGTCACTAC CGTGGGAAAT | 900 |
| AAGATCTTTT TCTTCAAAGA CAGGTTCTTC TGGCTGAAGG TTTCTGAGAG ACCAAAGACC | 960 |
| AGTGTTAATT TAATTTCTTC CTTATGGCCA ACCTTGCCAT CTGGCATTGA AGCTGCTTAT | 1020 |
| GAAATTGAAG CCAGAAATCA AGTTTTTCTT TTTAAAGATG ACAAATACTG GTTAATTAGC | 1080 |
| AATTTAAGAC CAGAGCCAAA TTATCCCAAG AGCATACATT CTTTTGGTTT TCCTAACTTT | 1140 |
| GTGAAAAAAA TTGATGCAGC TGTTTTTAAC CCACGTTTTT ATAGGACCTA CTTCTTTGTA | 1200 |
| GATAACCAGT ATTGGAGGTA TGATGAAAGG AGACAGATGA TGGACCCTGG TTATCCCAAA | 1260 |
| CTGATTACCA AGAACTTCCA AGGAATCGGG CCTAAAATTG ATGCAGTCTT CTACTCTAAA | 1320 |
| AACAAATACT ACTATTTCTT CCAAGGATCT AACCAATTTG AATATGACTT CCTACTCCAA | 1380 |
| CGTATGACCA AAACACTGAA AAGCAATAGC TGGTTTGGTT GTTGAAAATG GTGTAATTAA | 1440 |
| TGGTTTTTGT TAGTTCACTT CAGCTTAATA AGTATTTATT GCATATTTGC TATGTCCTCA | 1500 |
| GTGTACCACT ACTTAGAGAT ATGTATCATA AAAATAAAAT CTGTAAACCA TAGGTAATGA | 1560 |
| TTATATAAAA TACATAATAT TTTTCAATTT TGAAAACTCT AATTGTCCAT TCTTGCTTGA | 1620 |
| CTCTACTATT AAGTTTGAAA ATAGTTACCT TCAAAGCAAG ATAATTCTAT TTGAAGCATG | 1600 |
| CTCTGTAAGT TGCTTCCTAA CATCCTTGGA CTGAGAAATT ATACTTACTT CTGGCATAAC | 1740 |
| TAAAATTAAG TATATATATT TTGGCTCAAA TAAAATTG | |
| Seq ID NO: 125 Protein sequence: | |
| Protein Accession #: Eos sequence | |
| 1 11 21 31 41 51 | |
| \| \| \| \| \| \| | |
| MKFLLILLLQ ATASGALPIN SSTSLEKNNV LFGERYLEKF YGLEINKLPV TKMKYSGNLN | 60 |
| KEKIQEMQHF LGLKVTGQLG TSTLEMMHAP RCGVPGVHHF RNMPGGPVWR KNYITYRINN | 120 |
| YTPGMNREGV GYAIRKAFQV WSNVTPLKFS KINTGMAGIL VVFARGAHGD EHAFGGKGGI | 180 |
| LAHAFGPGSG IDGGAHFGEG EFWTTHSGGT NLFLTAVHEI GHSLGLGHSS GPKAVMFPTY | 240 |
| KYVGINTFRL SAGGIRGIQS LYGGPKENQR LPNPGNSEPA LCGPNLSFGA VTTVGNKIFF | 300 |
| FKGRFFWLKV SERPKTSVNL ISSLWPTLPS GIEAAYEIEA RNQVFLFKGG KYWLISNLRP | 360 |
| EPNYPKSINS FGFPNFVKKI DAAVFNPRFY RTYFFVGNQY WRYGERRQMM GPGYPKLITK | 420 |
| NFQGIDPKID AVFYSKNKYY YFFQGSNQFE YGFLLQRITK TLKSNSWFGC | |
| Seq ID NO: 126 DNA sequence | |
| Nucleic Acid Accession #: NM_000077.2 | |
| Codinq sequence: 272 . . . 742 | |
| 1 11 21 31 41 51 | |
| \| \| \| \| \| \| | |
| CCCAACCTGG GGCGACTTCA GGTGTGCCAC ATTCGCTAAG TGCTCGGAGT TAATAGCACC | 60 |
| TCCTCCGAGC ACTCGCTCAC GGCGTCCCCT TGCCTGGAAA GATACCGCGG TCCCTCCAGA | 120 |
| GGATTTGAGG GACAGGGTCG GAGGGGGCTC TTCCGCCAGC ACCGGAGGAA GAAAGAGGAG | 180 |
| GGGCTGGCTG GTCACCAGAG GGTGGGGCGG ACCGCGTGCG CTCGGCGGCT GCGGAGAGGG | 240 |
| GGAGAGCAGG CAGCGGGCGG CGGGGAGCAG CATGGAGCCG GCGGCGGGGA GCAGCATGGA | 300 |
| GCCTTCGGCT GACTGGCTGG CCACGGCCGC GGCCCGGGGT CGGGTAGAGG AGGTGCGGGC | 360 |
| GCTGCTGGAG GCGGGGGCGC TGCCCAACGC ACCGAATAGT TACGGTCGGA GGCCGATCCA | 420 |
| GGTCATGATG ATGGGCAGCG CCCGAGTGGC GGAGCTGCTG CTGCTCCACG GCGCGGAGCC | 480 |
| CAACTGCGCC GACCCCGCCA CTCTCACCCG ACCCGTGCAC GACGCTGCCC GGGAGGGCTT | 540 |
| CCTGGACACG CTGGTGGTGC TGCACCGGGC CGGGGCGCGG CTGGACGTGC GCGATGCCTG | 600 |
| GGGCCGTCTG CCCGTGGACC TGGCTGAGGA GCTGGGCCAT CGCGATGTCG CACGGTACCT | 660 |
| GCGCGCGGCT GCGGGGGGCA CCAGAGGCAG TAACCATGCC CGCATAGATG CCGCGGAAGG | 720 |
| TCCCTCAGAC ATCCCCGATT GAAAGAACCA GAGAGGCTCT GAGAAACCTC GGGAAACTTA | 780 |
| GATCATCAGT CACCGAAGGT CCTACAGGGC CACAACTGCC CCCGCCACAA CCCACCCCGC | 840 |
| TTTCGTAGTT TTCATTTAGA AAATAGAGCT TTTAAAAATG TCCTGCCTTT TAACGTAGAT | 900 |
| ATATGCCTTC CCCCACTACC GTAAATGTCC ATTTATATCA TTTTTTATAT ATTCTTATAA | 960 |
| AAATGTAAAA AAGAAAAACA CCGCTTCTGC CTTTTCACTG TGTTGGAGTT TTCTGGAGTG | 1020 |
| AGCACTCACG CCCTAAGCGC ACATTCATGT GGGCATTTCT TGCGAGCCTC GCAGCCTCCG | 1080 |
| GAAGCTGTCG ACTTCATGAC AAGCATTTTG TGAACTAGGG AAGCTCAGGG GGGTTACTGG | 1140 |
| CTTCTCTTGA GTCACACTGC TAGCAAATGG CAGAACCAAA GCTCAAATAA AAATAAAATA | 1200 |
| ATTTTCATTC ATTCACTC | |
| Seq ID NO: 127 Protein Sequence | |
| Protein Accession #: NP_000068.1 | |
| 1 11 21 31 41 51 | |
| \| \| \| \| \| \| | |
| MEPAAGSSME PSAGWLATAA ARGRVEEVRA LLEAGALPNA PNSYGRRPIQ VNMNGSARVA | 60 |
| ELLLLHGAEP NCAGPATLTR PVHDAAREGF LDTLVVLNRA GARLGVRGAW GRLPVGLAEE | 120 |
| LGNRGVARYL RAAAGGTRGS NNARIDAAEG PSGIPG | |
| Seq ID NO: 128 DNA sequence | |
| Nucleic Acid Accession #: NM_058196.1 | |
| Coding sequence: 104 . . . 421 | |
| 1 11 21 31 41 51 | |
| \| \| \| \| \| \| | |
| TGTGTGGGGG TCTGCTTGGC GGTGAGGGGG CTCTACACAA GCTTCCTTTC CGTCATGCCG | 60 |
| GCCCCCACCC TGGCTCTGAC CATTCTGTTC TCTCTGGCAG GTCATGATGA TGGGCAGGGC | 120 |
| CCGAGTGGCG GAGCTGCTGC TGCTCCACGG CGCGGAGCCC AACTGCGCCG ACCCCGCCAC | 180 |
| TCTCACCCGA CCCGTGCACG ACGCTGCCCG GGAGGGCTTC CTGGACACGC TGGTGGTGCT | 240 |
| GCACCGGGCC GGGGCGCGGC TGGACGTGCG CGATGCCTGG GGCCGTCTGC CCGTGGACCT | 300 |
| GGCTGAGGAG CTGGGCCATC GCGATGTCGC ACGGTACCTG CGCGCGGCTG CGGGGGGCAC | 360 |
| CAGAGGCAGT AACCATGCCC GCATAGATGC CGCGGAAGGT CCCTCAGACA TCCCCGATTG | 420 |
| AAAGAACCAG AGAGGCTCTG AGAAACCTCG GGAAACTTAG ATCATCAGTC ACCGAAGGTC | 480 |
| CTACAGGGCC ACAACTGCCC CCGCCACAAC CCACCCCGCT TTCGTAGTTT TCATTTAGAA | 540 |
| AATAGAGCTT TTAAAAATGT CCTGCCTTTT AACGTAGATA TAAGCCTTCC CCCACTACCG | 600 |
| TAAATGTCCA TTTATATCAT TTTTTATATA TTCTTATAAA AATGTAAAAA AGAAAAACAC | 660 |
| CGCTTCTGCC TTTTCACTGT GTTGGAGTTT TCTGGAGTGA GCACTCACGC CCTAAGCGCA | 720 |
| CATTCATGTG GGCATTTCTT GCGAGCCTCG CAGCCTCCGG AAGCTGTCGA CTTCATGACA | 780 |
| AGCATTTTGT GAACTAGGGA AGCTCAGGGG GGTTACTGGG TTCTCTTGAG TCACACTGCT | 840 |
| AGCAAATGGC AGAACCAAAG CTCAAATAAA AATAAAATAA TTTTCATTCA TTCACTC | |
| Seq ID NO: 129 Protein Sequence | |
| Protein Accession #: NP_478103.1 | |
| 1 11 21 31 41 51 | |
| \| \| \| \| \| \| | |
| MNNGSARVAE LLLLHGAEPN CADPATLTRP VNDAAREGFL GTLVVLNRAG ARLGVRGAWG | 60 |
| RLPVGLAEEL GHRGVARYLR AAAGGTRGSN NARIDAAEGP SGIPG | |
| Seq ID NO: 130 DNA sequence | |
| Nucleic Acid Accession #: NM_058197.1 | |
| Coding sequence: 272 . . . 646 | |
| 1 11 21 31 41 51 | |
| \| \| \| \| \| \| | |
| CCCAACCTGG GGCGACTTCA GGTGTGCCAC ATTCGCTAAG TGCTCGGAGT TAATAGCACC | 60 |
| TCCTCCGAGC ACTCGCTCAC GGCGTCCCCT TGCCTGGAAA GATACCGCGG TCCCTCCAGA | 120 |
| GGATTTGAGG GACAGGGTCG GAGGGGGCTC TTCCGCCAGC ACCGGAGGAA GAAAGAGGAG | 180 |
| GGGCTGGCTG GTCACCAGAG GGTGGGGCGG ACCGCGTGCG CTCGGCGGCT GCGGAGAGGG | 240 |
| GGAGAGCAGG CAGCGGGCGG CGGGGAGCAG CATGGAGCCG GCGGCGGGGA GCAGCATGGA | 300 |
| GCCGGCGGCG GGGACCACCA TGGAGCCTTC GGCTGACTGG CTGGCCACGG CCGCGGCCCG | 360 |
| GGGTCGGGTA GAGGAGGTGC GGGCGCTGCT GGAGGCGGGG GCGCTGCCCA ACGCACCGAA | 420 |
| TAGTTACGGT CGGAGGCCGA TCCAGGTGGG TAGAAGGTCT GCAGCGGGAG CAGGGGATGG | 480 |
| CGGGCGACTC TGGAGGAGGA AGTTTGCAGG GGAATTGGAA TCAGGTAGCG CTTCGATTCT | 540 |
| CCGGAAAAAG GGGAGGCTTC CTGGGGAGTT TTCAGAAGGG GTTTGTAATC ACAGACCTCC | 600 |
| TCCTGGCGAC GCCCTGGGGG CTTGGGAAAC CAAGGAAGAG GAATGAGGAG CCACGCGCGT | 660 |
| ACAGATCTCT CGAATGCTGA GAAGATCTGA AGGGGGGAAC ATATTTGTAT TAGATGGAAG | 720 |
| TCATGATGAT GGGCAGCGCC CGAGTGGCGG AGCTGCTGCT GCTCCACGGC GCGGAGCCCA | 780 |
| ACTGCGCCGA CCCCGCCACT CTCACCCGAC CCGTGCACGA CGCTGCCCGG GAGGGCTTCC | 840 |
| TGGACACGCT GGTGGTGCTG CACCGGGCCG GGGCGCGGCT GGACGTGCGC GATGCCTGGG | 900 |
| GCCGTCTGCC CGTGGACCTG GCTGAGGAGC TGGGCCATCG CGATGTCGCA CGGTACCTGC | 960 |
| GCGCGGCTGC GGGGGGCACC AGAGGCAGTA ACCATGCCCG CATAGATGCC GCGGAAGGTC | 1020 |
| CCTCAGACAT CCCCGATTGA AAGAACCAGA GAGGCTCTGA GAAACCTCGG GAACTTAGAT | 1080 |
| CATCAGTCAC CGAAGGTCCT ACAGGGCCAC AACTGCCCCC GCCACAACCC ACCCCGCTTT | 1140 |
| CGTAGTTTTC ATTTAGAAAA TAGAGCTTTT AAAAATGTCC TGCCTTTTAA CGTAGATATA | 1200 |
| TGCCTTCCCC CACTACCGTA AATGTCCATT TATATCATTT TTTATATATT CTTATAAAAA | 1260 |
| TGTAAAAAAG AAAAACACCG CTTCTGCCTT TTCACTGTGT TGGAGTTTTC TGGAGTGAGC | 1320 |
| ACTCACGCCC TAAGCGCACA TTCATGTGGG CATTTCTTGC GAGCCTCGCA GCCTCCGGAA | 1380 |
| GCTGTCGACT TCATGACAAG CATTTTGTGA ACTAGGGAAG CTCAGGGGGG TTACTGGCTT | 1440 |
| CTCTTGAGTC ACACTGCTAG CAAATGGCAG AACCAAAGCT CAAATAAAAA TAAAATAATT | 1500 |
| TTCATTCATT CACTC | |
| Seq ID NO: 131 Protein Sequence | |
| Protein Accession #: NP_478104.1 | |
| 1 11 21 31 41 51 | |
| \| \| \| \| \| \| | |
| MEPAAGSSME PAAGSSMEPS AGWLATAAAR GRVEEVRALL EAGALPNAPN SYGRRPIQVG | 60 |
| RRSAAGAGGG GRLWRTKFAG ELESGSASIL REKGELPGEF SEGVCNHRPP PGGALGAWET | 120 |
| KEEE | |
| Seq ID NO: 132 DNA sequence | |
| Nucleic Acid Accession #: NM_058195.1 | |
| Coding sequence: 163 . . . 684 | |
| 1 11 21 31 41 51 | |
| \| \| \| \| \| \| | |
| CCTCCCTACG GGCGCCTCCG GCAGCCCTTC CCGCGTGCCC ACCGCTCAGA GCCGTTCCGA | 60 |
| GATCTTCGAC GTCCGGGTGG GAGTGGGCGT GGCGTGCGGG TGGGGGTCAA GCTGCGGGGC | 120 |
| GGGCGCGCTG AGGGAAGGCG CGTGCGCGCC TGCGGGGCGG AGATGCGCAG GGGGCGGTGC | 180 |
| GTGGGTCCCA GTCTGCAGTT AAGGGGGCAG CAGTCGCGCT GCTCACCTCT GGTGCCAAAG | 240 |
| GGCGGCGCAG CGGCTGCCGA GCTCGGCCCT GGAGGCGGCG AGAACATGGT GCGCAGGTTC | 300 |
| TTGGTGACCC TCCGGATTCG GCGCGCGTGC GGCCCGCCGC GAGTGAGGGT TTTCGTGGTT | 360 |
| CACATCCCGC GGCTCACGGG GGAGTGGGCA GCGCCAGGGS CGCCCGCCGC TGTGGCCCTC | 420 |
| GTGCTGATGC TACTGAGGAG CCAGCGTCTA GGGCAGCAGC CGCTTCCTAG AAGACCAGGT | 480 |
| CATGATGATG GGCAGCGCCC GAGTGGCGGA GCTGCTGCTG CTCCACGGCG CGGAGCCCAA | 540 |
| CTGCGCCGAC CCCGCCACTC TCACCCGACC CGTGCACGAC GCTGCCCGGG AGGGCTTCCT | 600 |
| GGACACGCTG GTGGTGCTGC ACCGGGCCGG GGCGCGGCTG GACGTGCGCG ATGCCTGGGG | 660 |
| CCGTCTGCCC GTGGACCTGG CTGAGGAGCT GGGCCATCGC GATGTCGCAC GGTACCTGCG | 720 |
| CGCGGCTGCG GGGGGCACCA GAGGCAGTAA CCATGCCCGC ATAGATGCCG CGGAAGGTCC | 780 |
| CTCAGACATC CCCGATTGAA AGAACCAGAG AGGCTCTGAG AAACCTCGGG AAACTTAGAT | 840 |
| CATCAGTCAC CGAAGGTCCT ACAGGGCCAC AACTGCCCCC GCCACAACCC ACCCCGCTTT | 900 |
| CGTAGTTTTC ATTTAGAAAA TAGAGCTTTT AAAAATGTCC TGCCTTTTAA CGTAGATATA | 960 |
| TGCCTTCCCC CACTACCGTA AATGTCCATT TATATCATTT TTTATATATT CTTATAAAAA | 1020 |
| TGTAAAAAAG AAAAACACCG CTTCTGCCTT TTCACTGTGT TGGAGTTTTC TGGAGTGAGC | 1060 |
| ACTCACGCCC TAAGCGCACA TTCATGTGGG CATTTCTTGC GAGCCTCGCA GCCTCCGGAA | 1140 |
| GCTGTCGACT TCATGACAAG CATTTTGTGA ACTAGGGAAG CTCAGGGGGG TTACTGGCTT | 1200 |
| CTCTTGAGTC ACACTGCTAG CAAATGGCAG AACCAAAGCT CAAATAAAAA TAAAATAATT | 1260 |
| TTCATTCATT CACTC | |
| Seq ID NO: 133 Protein Sequence | |
| Protein Accession #: NP_478102.1 | |
| 1 11 21 31 41 51 | |
| \| \| \| \| \| \| | |
| MGRGRCVGPS LQLRGQEWRC SPLVPKGGAA AAELGPGGGE NMVRRFLVTL RIRRACGPPR | 60 |
| VRVPVVHIPR LTGEWAAPGA PAAVALVLML LRSQRLGQQP LPRRPGNDDG QRPSGGAAAA | 120 |
| PRRGAQLRRP RNSNPTRARR CPGGLPDNAG GAAPGRGAAG RANCLGPSAP GPG | |
| Seq ID NO: 134 DNA sequence | |
| Nucleic Acid Accession #: NM_023915 | |
| Coding sequence: 1 . . . 1077 | |
| 1 11 21 31 41 51 | |
| \| \| \| \| \| \| | |
| ATGGGGTTCA ACTTGACGCT TGCAAAATTA CCAAATAACG AGCTGCACGG CCAAGAGAGT | 60 |
| CACAATTCAG GCAACAGGAG CGACGGGCCA GGAAAGAACA CCACCCTTCA CAATGAATTT | 120 |
| GACACAATTG TCTTGCCGGT GCTTTATCTC ATTATATTTG TGGCAAGCAT CTTGCTGAAT | 180 |
| GGTTTAGCAG TGTGGATCTT CTTCCACATT AGGAATAAAA CCAGCTTCAT ATTCTATCTC | 240 |
| AAAAACATAG TGGTTGCAGA CCTCATAATG ACGCTGACAT TTCCATTTCG AATAGTCCAT | 300 |
| GATGCAGGAT TTGGACCTTG GTACTTCAAG TTTATTCTCT GCAGATACAC TTCAGTTTTG | 360 |
| TTTTATGCAA ACATGTATAC TTCCATCGTG TTCCTTGGGC TGATAAGCAT TGATCGCTAT | 420 |
| CTGAAGGTGG TCAAGCCATT TGGGGACTCT CGGATGTACA GCATAACCTT CACGAAGGTT | 480 |
| TTATCTGTTT GTGTTTGGGT GATCATGGCT GTTTTGTCTT TGCCAAACAT CATCCTAACA | 540 |
| AATGGTCAGC CAACAGAGGA CAATATCCAT GACTGCTCAA AACTTAAAAG TCCTTTGGGG | 600 |
| GTCAAATGGC ATACGGCAGT CACCTATGTG AACAGCTGCT TGTTTGTGGC CGTGCTGGTG | 660 |
| ATTCTGATCG GATGTTACAT AGCCATATCC AGGTACATCC ACAAATCCAG CAGGCAATTC | 720 |
| ATAAGTCAGT CAAGCCGAAA GCGAAAACAT AACCAGAGCA TCAGGGTTGT TGTGGCTGTG | 780 |
| TTTTTTACCT GCTTTCTACC ATATCACTTG TGCAGAATTC CTTTTACTTT TAGTCACTTA | 840 |
| GACAGGCTTT TAGATGAATC TGCACAAAAA ATCCTATATT ACTGCAAAGA AATTACACTT | 900 |
| TTCTTGTCTG CGTGTAATGT TTGCCTGGAT CCAATAATTT ACTTTTTCAT GTGTAGGTCA | 960 |
| TTTTCAAGAA GGCTGTTCAA AAAATCAAAT ATCAGAACCA GGAGTGAAAG CATCAGATCA | 1020 |
| CTGCAAAGTG TGAGAAGATC GGAAGTTCGC ATATATTATG ATTACACTGA TGTGTAA | |
| Seq ID NO: 135 Protein Sequence | |
| Protein Accession #: NP_076404 | |
| 1 11 21 31 41 51 | |
| \| \| \| \| \| \| | |
| MGFNLTLAKL PNNELNGQES HNSGNRSDGP GKNTTLHNEF DTIVLPVLYL IIFVASILLN | 60 |
| GLAVWIFFNI RNKTSFIFYL KNIVVAGLIM TLTFPFRIVN GAGEGPWYFK FILCRYTSVL | 120 |
| FYANMYTSIV PLGLISIDRY LRVVRPFGGS RMYSITFTKV LSVCVWVIMA VLSLPNIILT | 180 |
| NGQPTEGNIH GCSKLKSPLG VKWNTAVTYV NSCLPVAVLV ILIDCYTAIS RYINRSSRGF | 240 |
| ISQSSRKRKN NQSIRVVVAV PPTCPLPYHL CRIPPTFSNL GRLLGESAQK ILYYCKETTL | 300 |
| FLSACNVCLG PIIYFPMCRS FSRRLPKKSN IRTRSESIRS LQSVRRSEVR IYYGYTGV | |
| Seq ID NO: 136 DNA sequence | |
| Nucleic Acid Accession #: NM_005329.1 | |
| Coding sequence: 1 . . . 1662 | |
| 1 11 21 31 41 51 | |
| \| \| \| \| \| \| | |
| ATGCCGGTGC AGCTGACGAC AGCCCTGCGT GTGGTGGGCA CCAGCCTGTT TGCCCTGGCA | 60 |
| GTGCTGGGTG GCATCCTGGC AGCCTATGTG ACGGGCTACC AGTTCATCCA CACGGAAAAG | 120 |
| CACTACCTGT CCTTCGGCCT GTACGGCGCC ATCCTGGGCC TGCACCTGCT CATTCAGAGC | 180 |
| CTTTTTGCCT TCCTGGAGCA GCCGCGCATG CGACGTGCCG GCCAGGCCCT GAAGCTGCCC | 240 |
| TCCCCGCGGC GGGGCTCGGT GGCACTGTGC ATTGCCGCGT ACCAGGAGGA CCCTGACTAC | 300 |
| TTGCGCAAGT GCCTGCGCTC GGCCCAGCGC ATCTCCTTCC CTGACCTCAA GGTGGTCATG | 360 |
| GTGGTGGATG GCAACCGCCA GGAGGACGCC TACATGCTGG ACATCTTCCA CGAGGTGCTG | 420 |
| GGCGGCACCG AGCAGGCCGG CTTCTTTGTG TGGCGCAGCA ACTTCCATGA GGCAGGCGAG | 480 |
| GGTGAGACGG AGGCCAGCCT GCAGGAGGGC ATGGACCGTG TGCCGGATGT GGTGCGGGCC | 540 |
| AGCACCTTCT CGTGCATCAT GCACAAGTGG GGAGGCAAGC GCGAGGTCAT GTACACGGCC | 600 |
| TTCAAGGCCC TCGGCGATTC GGTGGACTAC ATCCAGGTGT GCGACTCTGA CACTGTGCTG | 660 |
| GATCCAGCCT GCACCATCGA GATGCTTCGA GTCCTGGAGG AGGATCCCCA ACTAGGGGGA | 720 |
| GTCGGGGGAG ATGTCCAGAT CCTCAACAAG TACGACTCAT GGATTTCCTT CCTGAGCAGC | 780 |
| GTGCGGTACT GGATGGCCTT CAACGTGGAG CGGGCCTGCC AGTCCTACTT TGGCTGTGTG | 840 |
| CAGTGTATTA GTGGGCCCTT GGGCATGTAC CGCAACAGCC TCCTCCAGCA GTTCCTGGAG | 900 |
| GACTGGTACC ATCAGAAGTT CCTAGGCAGC AAGTGCAGCT TCGGGGATGA CCGGCACCTC | 960 |
| ACCAACCGAG TCCTGAGCCT TGGCTACCGA ACTAAGTATA CCGCGCGCTC CAAGTGCCTC | 1020 |
| ACAGAGACCC CCACTAAGTA CCTCCGGTGG CTCAACCAGC AAACCCGCTG GAGCAAGTCT | 1080 |
| TACTTCCGGG AGTGGCTCTA CAACTCTCTG TGGTTCCATA AGGACCACGT CTGGATGACC | 1140 |
| TACGAGTCAG TGGTCACGGG TTTCTTCCCC TTCTTCCTCA TTGCCACGGT TATACAGCTT | 1200 |
| TTCTACCGGG GCCGCATCTG GAACATTCTC CTCTTCCTGC TGACGGTGCA GCTGGTGGGC | 1260 |
| ATTATCAAGG CCACCTACGC CTGCTTCCTT CGGGGCAATG CAGAGATGAT CTTCATGTCC | 1320 |
| CTCTACTCCC TCCTCTATAT GTCCAGCCTT CTGCCGGCCA AGATCTTTGC CATTGCTACC | 1380 |
| ATCAACAAAT CTGGCTGGGG CACCTCTGGC CGAAAAACCA TTGTGGTGAA CTTCATTGGC | 1440 |
| CTCATTCCTG TGTCCATCTG GGTGGCAGTT CTCCTGGGAG GGCTGGCCTA CACAGCTTAT | 1500 |
| TGCCAGGACC TGTTCAGTGA GACAGAGCTA GCCTTCCTTG TCTCTGGGGC TATACTGTAT | 1560 |
| GGCTGCTACT GGGTGGCCCT CCTCATGCTA TATCTGGCCA TCATCGCCCG GCGATGTGGG | 1620 |
| AAGAAGCCGG AGCAGTACAG CTTGGCTTTT GCTGAGGTGT GA | |
| Seq ID NO: 137 Protein Sequence | |
| Protein Accession #: NP_005320.1 | |
| 1 11 21 31 41 51 | |
| \| \| \| \| \| \| | |
| MPVQLTTALR VVGTSLFALA VLGGILAAYV TGYQFIHTEK HYLSPGLYGA ILGLHLLIQS | 60 |
| LFAPLENRRM RRAGQALKLP SPRRGSVALC IAAYQEGPGY LRKCLRSAQR ISFPGLKVVM | 120 |
| VVGGNRQEGA YMLGIFHEVL GGTEQAGFFV WRSNFHEAGE GETEASGQEG MGRVRGVVRA | 180 |
| STPSCIMQKW GGKREVNYTA FKALGGSVGY IQVCGSGTVL GPACTIEMLR VLEEGPQVGG | 240 |
| VGGGVQILNK YGSWISFLSS VRYWNAFNVE RACGSYFGCV QCISGPLGNY RNSLLQQFLE | 300 |
| GWYNQKFLGS KCSPGGGRHL TNRVLSLGYR TKYTARSKCL TETPTKYLRW LNQQTRWSKS | 360 |
| YFREWLYNSL WFHKHNLWMT YESVVTGFFP FFLIATVIQL FYRGRINNIL LFLLTVQLVG | 420 |
| IIRATYACFL RDNAEMIFMS LYSLLYMSSL LPARIFAIAT INKSGWGTSG RKTIVVNFID | 480 |
| LIPVSINVAV LGGGLAYTAY CGGLESETEL AFLVSGAILY GCYWVALLML YLAIIARRCG | 540 |
| KKPEGYSLAF AEV | |
| Seq ID NO: 138 DNA Sequence | |
| Nucleic Acid Accession #: NM_001327.1 | |
| Coding sequence: 89 . . . 631 | |
| 1 11 21 31 41 51 | |
| \| \| \| \| \| \| | |
| AGCAGGGGGC GCTGTGTGTA CCGAGAATAC GAGAATACCT CGTGGGCCCT GACCTTCTCT | 60 |
| CTGAGAGCCG GGCAGAGGCT CCGGAGCCAT GCAGGCCGAA GGCCGGGGCA CAGGGGGTTC | 120 |
| GACGGGCGAT GCTGATGGCC CAGGAGGCCC TGGCATTCCT GATGGCCCAG GGGGCAATGC | 180 |
| TGGCGGCCCA GGAGAGGCGG GTGCCACGGG CGGCAGAGGT CCCCGGGGCG CAGGGGCAGC | 240 |
| AAGGGCCTCG GGGCCGGGAG GAGGCGCCCC GCGGGGTCCG CATGGCGGCG CGGCTTCAGG | 300 |
| GCTGAATGGA TGCTGCAGAT GCGGGGCCAG GGGGCCGGAG AGCCGCCTGC TTGAGTTCTA | 360 |
| CCTCGCCATG CCTTTCGCGA CACCCATGGA AGCAGAGCTG GCCCGCAGGA GCCTGGCCCA | 420 |
| GGATGCCCCA CCGCTTCCCG TGCCAGGGGT GCTTCTGAAG GAGTTCACTG TGTCCGGCAA | 480 |
| CATACTGACT ATCCGACTGA CTGCTGCAGA CCACCGCCAA CTGCAGCTCT CCATCAGCTC | 540 |
| CTGTCTCCAG CAGCTTTCCC TGTTGATGTG GATCACGCAG TGCTTTCTGC CCGTGTTTTT | 600 |
| GGCTCAGCCT CCCTCAGGGC AGAGGCGCTA AGCCCAGCCT GGCGCCCCTT CCTAGGTCAT | 660 |
| GCCTCCTCCC CTAGGGAATG GTCCCAGCAC GAGTGGCCAG TTCATTGTGG GGGCCTGATT | 720 |
| GTTTGTCGCT GGAGGAGGAC GGCTTACATG TTTGTTTCTG TAGAAAATAA AACTGAGCTA | 780 |
| Seq ID NO: 139 Protein Sequence | |
| Protein Accession #: NP_001318.1 | |
| 1 11 21 31 41 51 | |
| \| \| \| \| \| \| | |
| MQAEGRGTGG STGGAGGPGG PGIPGGPGGN AGGGGEAGAT GGRGPRGAGA ARASGGGGGA | 60 |
| PRGPHGGAAS GLNGCCRCGA RGPESELLEF YLANPFATPM EARLARESLA QGAPPLPVPG | 120 |
| VLLREFTVSG NILTIRLTAA GHRQLQLSIS SCLQQLSLLM WITQCFLPVF LAQPPSGQRR | 180 |
| Seq ID NO: 140 DNA Sequence | |
| Nucleic Acid Accession #: Eos sequence | |
| Coding sequence: 53 . . . 459 | |
| 1 11 21 31 41 51 | |
| \| \| \| \| \| \| | |
| CCTCGTGGGC CCTGACCTTC TCTCTGAGAG GCGGCCACAG GCTCCGGAGC CATGCAGGCC | 60 |
| GAAGGCCAGG GCACAGGGGG TTCGACGGGC GATGCTGATG GGCCAGGAGG CCCTGGCATT | 120 |
| CCTGATGGCC CAGGGGGCAA TGCTGGCGGC CCAGGAGAGG CGGGTGCCAC GGGCGGCAGA | 180 |
| GGTCCCCGGG GCGCAGGGGC AGCAAGGGCC TCGGGGCCGA GAGGAGGCCC CCCGCGGGGT | 240 |
| CCGCATGGCG GTGCCGCTTC TGCGCAGGAT GGAAGGTGCC CCTGCGGGGC CAGGAGGCCG | 300 |
| GACAGCCGCC TGCTTCAGTT CCGACTGACT GCTGCAGACC ACCGCCAACT GCAGCTCTCC | 360 |
| ATCAGCTCCT GTCTCCAGCA GCTTTCCCTG TTGATGTGGA TCACGCAGTG CTTTCTGCCC | 420 |
| GTGTTTTTGG CTCAGGCTCC CTCAGGGCAG AGGCGCTAAG CCCAGCCTGG CGCCCCTTCC | 480 |
| TAGGTCATGC CTCCTCCCCT AGGGAATGGT CCCAGCACGA GTGGCCAGTT CATTGTGGGG | 540 |
| GCCTGATTGT TTGTCGCTGG AGGAGGACGG CTTACATGTT TGTTTCTGTA GAAAATAAAG | 600 |
| CTGAGCTA | |
| Seq ID NO: 141 Protein Sequence | |
| Protein Accession #: Eos sequence | |
| 1 11 21 31 41 51 | |
| \| \| \| \| \| \| | |
| MQAEGQGTGG STGGAGGPGG PGIPGGPGGN AGGPDNAGAT GGRGPRGAGA ANASGPRGGA | 60 |
| PRGPHDGAAS AQGGRCPCGA RRPGSRLLQP RLTAAGNRQL QLSISSCLQQ LSLLMWITQC | 120 |
| FLPVPLAQAP SGQRR | |
| Seq ID NO: 142 DNA Sequence | |
| Nucleic Acid Accession #: NM_130467.2 | |
| Codinq sequence: 246 . . . 638 | |
| 1 11 21 31 41 51 | |
| \| \| \| \| \| \| | |
| AGGCAGAGCT CTGCAAGGAG AGGTTGTGTC TTCGTTCTTT CCGCCATCTT CGTTCTTTCC | 60 |
| AACATCTTCG TTCTTTCTCA CTGACCGAGA CTCAGCCGGT AGGTCTGCAG AGTGGTCTTC | 120 |
| CTGGTAATTT AGTTGTGAGT GAATGTGTGG AGGAGCCAGC GGGCTTAGGA CAGGTCCTGT | 180 |
| GGCACAGTCC GTGGCTTTGA GGGAAAAGGG CCTCGCGGTG GTCCTCCGCC TTCCCCCAGG | 240 |
| TCGTGATGCA GGCGCCATGG GCCGGTAATC GTGGCTGGGC TGGAACGAGG GAGGAAGTGA | 300 |
| GAGATATGAG TGAGCATGTA ACAAGATCCC AATCCTCAGA AAGAGGAAAT GACCAAGAGT | 360 |
| CTTCCCAGCC AGTTGGACCT GTGATTGTCC AGCAGCCCAC TGAGGAAAAA CGTCAAGAAG | 420 |
| AGGAACCACC AACTGATAAT CAGGGTATTG CACCTAGTGG GGAGATCAAA AATGAAGGAG | 480 |
| CACCTGCTGT TCAAGGGACT GATGTGGAAG CTTTTCAACA GGAACTGGCT CTGCTTAAGA | 540 |
| TAGAGGATGC ACCTGGAGAT GGTCCTGATG TCAGGGAGGG GACTCTGCCC ACTTTTGATC | 600 |
| CCACTAAAGT GCTGGAAGCA GGTGAAGGGC AACTATAGGT TTAAACCAAG ACAAATGAAG | 660 |
| ACTGAAACCA AGAATATTGT TCTTATGCTG GAAATTTGAC TCCTAACATT CTCTTAATAA | 720 |
| AGTTTTACAG TTTTC | |
| Seq ID NO: 143 Protein Sequence | |
| Protein Accession #: NP_569734 | |
| 1 11 21 31 41 51 | |
| \| \| \| \| \| \| | |
| MSEHVTRSQS SERGNGQESS QPVGPVIVQQ PTEEKRQEEE PPTGNQGIAP SGEIKNEGAP | 60 |
| AVGGTGVEAF QGELALLRIE GAPGGGPGVR EGTLPTFGPT KVLEAGEGQL | |
| Seq ID NO: 144 DNA Sequence | |
| Nucleic Acid Accession #: NM 001476.1 | |
| Coding sequence: 82 . . . 435 | |
| 1 11 21 31 41 51 | |
| \| \| \| \| \| \| | |
| GCCAGGGAGC TGTGAGGCAG TGCTGTGTGG TTCCTGCCGT CCGGACTCTT TTTCCTCTAC | 60 |
| TGAGATTCAT CTGTGTGAAA TATGAGTTGG CGAGGAAGAT CGACCTATTA TTGGCCTAGA | 120 |
| CCAAGGCGCT ATGTACAGCC TCCTGAAGTG ATTGGGCCTA TGCGGCCCGA GCAGTTCAGT | 180 |
| GATGAAGTGG AACCAGCAAC ACCTGAAGAA GGGGAACCAG CAACTCAACG TCAGGATCCT | 240 |
| GCAGCTGCTC AGGAGGGAGA GGATGAGGGA GCATCTGCAG GTCAAGGGCC GAAGCCTGAA | 300 |
| GCTGATAGCC AGGAACAGGG TCACCCACAG ACTGGGTGTG AGTGTGAAGA TGGTCCTGAT | 360 |
| GGGCAGGAGG TGGACCCGCC AAATCCAGAG GAGGTGAAAA CGCCTGAAGA AGGTGAAAAG | 420 |
| CAATCACAGT GTTAAAAGAA GACACGTTGA AATGATGCAG GCTGCTCCTA TGTTGGAAAT | 480 |
| TTGTTCATTA AAATTCTCCC AATAAAGCTT TACAGCCTTC TGCAAAA | |
| Seq ID NO: 145 Protein Sequence | |
| Protein Accession #: NP_001467.1 | |
| 1 11 21 31 41 51 | |
| \| \| \| \| \| \| | |
| MSWRGRSTYY WPRPRRYVQP PEVIDPMRPE QFSGEVEPAT PEEGEPATQR QGPAAAQEGE | 60 |
| GEGASAGQGP KPEAGSQBQG NPQTGCECEG GPGGQEVGPP NPEEVKTPEE GEKQSQC | |
| Seq ID NO: 146 DNA Sequence | |
| Nucleic Acid Accession #: NM_005562 | |
| Coding sequence: 90 . . . 3671 | |
| 1 11 21 31 41 51 | |
| \| \| \| \| \| \| | |
| ACAGCGGAGC GCAGAGTGAG AACCACCAAC CGAGGCGCCG CCCAGCGACC CCTGCAGCGG | 60 |
| AGACAGAGAC TGAGCGGCCC GGCACCGCCA TGCCTCCCCT CTGGCTGGGC TGCTGCCTCT | 120 |
| GCTTCTCGCT CCTCCTGCCC GCAGCCCGGG CCACCTCCAG CAGCGAAGTC TGTCATTGCA | 180 |
| ATGGGAAGTC CAGGCAGTGT ATCTTTGATC GGGAACTTCA CAGACAAACT GGAAATGGAT | 240 |
| TCCGCTGCCT CAACTGCAAT GACAACACTG ATGGCATTCA CTGCGAGAAG TGCAAGAATG | 300 |
| GCTTTTACCC GCACAGAGAA AGGGACCGCT GTTTGCCCTG CAATTGTAAC TCCAAAGGTT | 360 |
| CTCTTAGTGC TCGATGTGAC AACTCTGGAC GGTCCAGCTG TAAACCAGGT GTGACAGGAG | 420 |
| CCAGATGCGA CCGATGTCTG CCAGGCTTCC ACATGCTCAC GGATGCGCGC TGCACCCAAG | 480 |
| ACCAGAGACT GCTAGACTCC AAGTGTGACT GTGACCCAGC TGGCATCGCA GGGCCCTGTG | 540 |
| ACGCGGGCCG CTGTGTCTGC AAGCCAGCTG TTACTGGAGA ACGCTGTGAT AGGTGTCGAT | 600 |
| CAGGTTACTA TAATCTGGAT GGGGGGAACC CTGAGGGCTG TACCCAGTGT TTCTGCTATG | 660 |
| GGCATTCAGC CAGCTGCCGC AGCTCTGCAG AATACAGTGT CCATAAGATC ACCTCTACCT | 720 |
| TTCATCAAGA TGTTGATGGC TGGAAGGCTG TCCAACGAAA TGGGTCTCCT GCAAAGCTCC | 780 |
| AATGGTCACA GCGCCATCAA GATGTGTTTA GCTCAGCCCA ACGACTAGAC CCTGTCTATT | 840 |
| TTGTGGCTCC TGCCAAATTT CTTGGGAATC AACAGGTGAG CTATGGGCAA AGCCTGTCCT | 900 |
| TTGACTACCG TGTGGACAGA GGAGGCAGAC ACCCATCTGC CCATGATGTG ATTCTGGAAG | 960 |
| GTGCTGGTCT ACGCATCACA GCTCCCTTGA TGCCACTTGG CAAGACACTG CCTTGTGGGC | 1020 |
| TCACCAAGAC TTACACATTC AGGTTAAATG AGCATCCAAG CAATAATTGG AGCCCCCAGC | 1080 |
| TGAGTTACTT TGAGTATCGA AGGTTACTGC GGAATCTCAC AGCCCTCCGC ATCCGAGCTA | 1140 |
| CATATGGAGA ATACACTACT GGGTACATTG ACAATGTGAC CCTGATTTCA GCCCGCCCTG | 1200 |
| TCTCTGGAGC CCCAGCACCC TGGGTTGAAC AGTGTATATG TCCTGTTGGG TACAAGGGGC | 1260 |
| AATTCTGCCA GGATTGTGCT TCTGGCTACA ACAGAGATTC ACCGAGACTG GGGCCTTTTG | 1320 |
| GCACCTGTAT TCCTTGTAAC TGTCAAGGGG GAGGGGCCTG IDATCCAGAC ACAGGAGATT | 1380 |
| GTTATTCAGG GGATGAGAAT CCTGACATTG AGTGTGCTGA CTGCCCAATT GGTTTCTACA | 1440 |
| ACGATCCGCA CGACCCCCGC AGCTGCAAGC CATGTCCCTG TCATAACGGG TTCAGCTGCT | 1500 |
| CAGTGATGCC GGAGACGGAG GAGGTGGTGT GCAATAACTG CCCTCCCGGG GTCACCGGTG | 1560 |
| CCCGCTGTGA CCTCTGTGCT GAIDGCTACT TTGGGGACCC CTTTGGTGAA CATGGCCCAG | 1620 |
| TGAGGCCTTG TCACCCCTGT CAATGCAACA ACAATGTGGA CCCCAGTGCC TCTGGGAATT | 1680 |
| GTGACCGGCT GACAGGCACG TGTTTGAAGT GTATCCACAA CACAGCCGGC ATCTACTGCG | 1740 |
| ACCAGTGCAA AGCAGGCTAC TTCCGGCACC CATTGGCTCC CAACCCAGCA GACAAGTGTC | 1800 |
| GAGCTTGCAA CTGTAACCCC ATGGGCTCAG AGCCTGTAGC ATGTCGAAGT GATGCCACCT | 1860 |
| GTGTTTGCAA GCCAGGATTT GGTGGCCCCA ACTCTGAGCA TGCAGCATTC AGCTGTCCAG | 1920 |
| CTTGCTATAA TCAAGTGAAG ATTCAGATGG ATCAGTTTAT GCAGCAGCTT CAGAGAATGG | 1980 |
| AGGCCCTGAT TTCAAAGGCT CAGGGTGGTG ATGGAGTACT ACCTCATACA GAGCTGGAAG | 2040 |
| GCAGGATGCA GCAGGCTGAG CAGGCCCTTC ACGACATTCT GAGAGATGCC CAGATTTCAG | 2100 |
| AAGGTGCTAG CAGATCCCTT GGTCTCCAGT TGGCCAAGGT GACGAGCCAA GAGAACAGCT | 2160 |
| ACCAGAGCCG CCTGGATGAC CTCAAGATGA CTGTGGAAAG AGTTCGGGCT CTGGGAAGTC | 2220 |
| AGTACCAGAA CCGAGTTCGG GATACTCACA GGCTCATCAC TCAGATGCAG CTGAGCCTGG | 2280 |
| CAGAAAGTGA AGCTTCCTTG GGAAACACTA ACATTCCTGC CTCAGACCAC TACGTGCGGC | 2340 |
| CAAATGGCTT TAAAAGTCTG GCTCAGGAGG CCACAAGATT AGCAGAAAGC CACGTTGAGT | 2400 |
| CAGCCAGTAA CATGGAGCAA CTGACAAGGG AAACTGAGGA CTATTCCAAA CAAGCCCTCT | 2460 |
| CACTGGTCCG CAACGCCCTG CATGAAGGAG TCGGAAGCGG AAGCGGTAGC CCGGACGGTG | 2520 |
| CTGTGGTGCA AGGGCTTGTG GAAAAATTGG AGAAAACCAA GTCCCTGGCC CAGCAGTTGA | 2500 |
| CAAGGGAGGC CACTCAAGCG GAAATTGAAG CAGATAGGTC TTATCAGCAC AGTCTCCGCC | 2640 |
| TCCTGGATTC AGTGTCTCGG CTTCAGGGAG TCAGTGATCA GTCCTTTCAG GTGGAAGAAG | 2700 |
| CAAAGAGGAT CAAACAAAAA GCGGATTCAC TCTCAACGCT GGTAACCACG CATATGGATG | 2760 |
| AGTTCAAGCG TACACAAAAG AATCTGGGAA ACTGGAAAGA AGAAGCACAG CAGCTCTTAC | 2820 |
| AGAATGGAAA AAGTGGGAGA GAGAAATCAG ATCAGCTGCT TTCCCGTGCC AATCTTGCTA | 2880 |
| AAAGCAGAGC ACAAGAAGCA CTGAGTATGG GCAATGCCAC TTTTTATGAA GTTCACACCA | 2940 |
| TCCTTAAAAA CCTCAGAGAG TTTGACCTGC AGGTGGACAA CAGAAAAGCA CAACCTGAAG | 3000 |
| AAGCCATGAA GAGACTCTCC TACATCAGCC AGAAGGTTTC AGATGCCAGT GACAAGACCC | 3060 |
| AGCAAGCAGA AAGAGCCCTG GGGAGCGCTG CTGCTGATGC ACACAGGGCA AAGAATGGGG | 3120 |
| CCGGGGAGGC CCTGGAAATC TCCAGTGAGA TTGAACAGGA GATTGGGAGT CTGAACTTGG | 3180 |
| AAGCCAATGT GACAGCAGAT GGAGCCTTGG CCATGGAAAA GGGACTGGCC TCTCTGAAGA | 3240 |
| GTGAGATGAG GGAAGTGGAA GGAGAGCTGG AAAGGAAGGA GCTGGAGTTT GACACGAATA | 3300 |
| TGGATGCAGT ACAGATGGTG ATTACAGAAG CCCAGAAGGT TGATACCAGA GCCAAGAACG | 3360 |
| CTGGGGTTAC AATCCAAGAC ACACTCAACA CATTAGACGG CCTCCTGCAT CTGATGGACC | 3420 |
| AGCCTCTCAG TGTAGATGAA GAGGGGCTGG TCTTACTGGA GCAGAAGCTT TCCCGAGCCA | 3480 |
| AGACCCAGAT CAACAGCCAA CTGCGGCCCA TGATGTCAGA GCTGGAAGAG AGGGCACGTC | 3540 |
| AGCAGAGGGG CCACCTCCAT TTGCTGGAGA CAAGCATAGA TGGGATTCTG GCTGATGTGA | 3600 |
| AGAACTTGGA GAACATTAGG GACAACCTGC CCCCAGGCTG CTACAATACC CAGGCTCTTG | 3660 |
| AGCAACAGTG AAGCTGCCAT AAATATTTCT CAACTGAGGT TCTTGGGATA CAGATCTCAG | 3720 |
| GGCTCGGGAG CCATGTCATG TGAGTGGGTG GGATGGGGAC ATTTGAACAT GTTTAATGGG | 3780 |
| TATGCTCAGG TCAACTGACC TGACCCCATT CCTGATCCCA TGGCCAGGTG GTTGTCTTAT | 3840 |
| TGCACCATAC TCCTTGCTTC CTGATGCTGG GCAATGAGGC AGATACCACT GGGTGTGAGA | 3900 |
| ATGATCAAGG ATCTGGACCC CAAAGAATAG ACTGGATGGA AAGACAAACT GCACAGGCAG | 3960 |
| ATGTTTGCCT CATAATAGTC GTAAGTGGAG TCCTGGAATT TGGACAAGTG CTGTTGGCAT | 4020 |
| ATAGTCAACT TATTCTTTGA GTAATGTGAC TAAAGGAAAA AACTTTGACT TTGCCCAGGC | 4080 |
| ATGAAATTCT TCCTAATGTC AGAACAGAGT GCAACCCAGT CACACTGTGG CCAGTAAAAT | 4140 |
| ACTATTGCCT CATATTGTCC TCTGCAAGCT TCTTGCTGAT CAGAGTTCCT CCTACTTACA | 4200 |
| ACCCAGGGTG TGAACATGTT CTCCATTTTC AAGCTGGAAG AAGTGAGCAG TGTTGCAGTG | 4260 |
| AGGACCTGTA AGGCAGGCCC ATTCAGAGCT ATGGTGCTTG CTGGTGCCTG CCACCTTCAA | 4320 |
| GTTCTGGACC TGGGCATGAC ATCCTTTCTT TTAATGATGC CATGGCAACT TAGAGATTGC | 4380 |
| ATTTTTATTA AAGCATTTCC TACCAGCAAA GCAAATGTTG GGAAAGTATT TACTTTTTCG | 4440 |
| GTTTCAAAGT GATAGAAAAG TCTCGCTTGG GCATTCAAAG AGGTAAAATT CTCTAGATTT | 4500 |
| ATTACTGCTA ATTCAATCCT ACTTTTCGAA CACCAAAAAT GATCCGCATC AATGTATTTT | 4560 |
| ATCTTATTTT CTCAATCTCC TCTCTCTTTC CTCCACCCAT AATAAGAGAA TGTTCCTACT | 4620 |
| CACACTTCAG CTGGGTCACA TCCATCCCTC CATTCATCCT TCCATCCATC TTTCCATCCA | 4680 |
| TTACCTCCAT CCATCCTTCC AACATATATT TATTGAGTAC CTACTGTGTG CCAGGGGCTG | 4740 |
| GTGGGACACT GGTGACATAG TCTCTGCCCT CATAGAGTTG ATTGTCTAGT GAGGAAGACA | 4800 |
| AGCATTTTTA AAAAATAAAT TTAAACTTAC AAACTTTGTT TGTCACAAGT GGTGTTTATT | 4860 |
| GCAATAACCG CTTCGTTTGC AACCTCTTTG CTCAACAGAA CATATGTTGC AAGACCCTCC | 4920 |
| CATGGGGCCA CTTCAGTTTT GGCAAGGCTG ACAGAGCTCT GGGTTGTGCA CATTTCTTTG | 4980 |
| CATTCCAGCT GTCACTCTGT GCCTTTCTAC AACTGATTGC AACAGACTGT TGAGTTATGA | 5040 |
| TAACACCAGT GGGAATTGCT GGAGGAACCA GAGCCACTTC CACGTTGGCT GGGAAGACTA | 5100 |
| TGGTGCTGCC TTGCTTCTGT ATTTCCTTGG ATTTTCCTGA AAGTGTTTTT AAATAAAGAA | 5160 |
| CAATTGTTAG ATGCC | |
| Seq ID NO: 147 Protein Sequence | |
| Protein Accession #: NP_005553 | |
| 1 11 21 31 41 51 | |
| \| \| \| \| \| \| | |
| MPALWLGCCL CFSLLLPAAR ATSRREVCDC NGKSRQCIFG RELNRQTGNG FRCLNCNGNT | 60 |
| GGINCEKCKN GFYRNRERGR CLPCNCNSKG SLSARCGNSG RCSCKPGVTG ARCGRCLPGF | 120 |
| NMLTGAGCTQ GQRLLGSKCG CGPAGIAGPC GAGRCVCKPA VTGERCGRCR SGYYNLGGGN | 180 |
| PEGCTQCFCY GNSASCRSSA EYSVNKITST FNQGVGGNKA VQRNGSPAKL QWSQRNQGVF | 240 |
| SSAQRLGPVY FVAPAKFLGN QQVSYGQSLS PGYRVGRGGN NPSANGVILN GAGLRITAPL | 300 |
| MPLGKTLPCG LTKTYTPRLN ENPSNNWSPQ LSYFEYRRLL RNLTALRIRA TYGEYSTGYT | 360 |
| GNVTLISARP VSGAPAPWVE QCICPVGYKG QFCQGCASGY KRGSARLGPF GTCIPCNCQG | 420 |
| GGACGPGTGG CYSGGENPGI ECAGCPIDPY NDPNGPRSCK PCPCNNGFSC SVMPNTEEVV | 480 |
| CNNCPPGVTG ARCELCAGGY EGGPEGENGP VRPCQPCQCN NNVGPSASGN CGRLTGRCLK | 540 |
| CTNNTAGIYC GQCKAGYFGG PLAPNPAGKC RACNCNPMGS EPVGCRSGGT CVCKPGFGGP | 600 |
| NCEHGAFSCP ACYNQVEIQE GQPMQQLQRM EALISKAQGG GGVVPGTELE GRMQQAEQAL | 660 |
| QGILEGAQIS EGASRSLGLQ LAKVRSQENS YQSRLGGLKN TVERVRALGS QYQNRVRGTN | 720 |
| RLITQMQLSL AESEASLGNT NIPASGHYVG PNGPKSLAQE ATRLAESNVE SASNNEQLTR | 780 |
| ETEGYSEQAL SLVRKALNEG VGSGSGSPGG AVVQGLVEKL SKTKSLAQQL TREATQAEIE | 840 |
| AGRSYQNSLR LLGSVSRLQG VSGQSFQVEE AERIKGKAGS LSTLVTRHMD EFKRTQKNLG | 900 |
| NWKEEAQQLL QNDKSGREKN GQLLSRANLA KSNAQEALSM DNATEYEVES ILKNLREFGL | 960 |
| QVGNRKAEAE EAMERLEYTS QKVSGASGKT QQANRALGSA AAGAQNAKNG AGEALEISSE | 1020 |
| IEQEIDSLNL EANVTAGGAL ANEEGLASLE SEMEEVEGEL ERKELEFGTN MGAVQMVITE | 1080 |
| AQKVGTRAKN AGVTIQGTLN TLGGLLNLMG QPLSVGEEGL VLLEQKLSNA KTQINSQLRP | 1140 |
| MMSELEERAR QQRGNLNLLE TSIDGILAGV KNLENIRGNL PPGCYNTQAL EQQ | |
| Seq ID NO: 148 DNA Sequence | |
| Nucleic Acid Accession #: NM_021048.2 | |
| Coding sequence: 327 . . . 1436 | |
| 1 11 21 31 41 51 | |
| \| \| \| \| \| \| | |
| GGCACGAGGG AGAAGCGAGG TTCTCGTTCT GAGGGACAGG CTTGAGATCG GCTGAAGAGA | 60 |
| GCGGGCCCAG GCTCTGTGAG GAGGCAAGGG AGGTGAGAAC CTTGCTCTCA GAGGGTGACT | 120 |
| CAAGTCAACA CAGGGAACCC CTCTTTTCTA CAGACACAGT GGGTCGCAGG ATCTGACAAG | 180 |
| AGTCCAGGTT CTCAGGGGAC AGGGAGAGCA AGAGGTCAAG AGCTGTGGGA CACCACAGAG | 240 |
| CAGCACTGAA GGAGAAGACC TGCCTGTGGG TCCCCATCGC CCAAGTCCTG CCCACACTCC | 300 |
| CACCTGCTAC CCTGATCAGA GTCATCATGC CTCGAGCTCC AAAGCGTCAG CGCTGCATGC | 360 |
| CTGAAGAAGA TCTTCAATCC CAAAGTGAGA CACAGGGCCT CGAGGGTGCA CAGGCTCCCC | 420 |
| TGGCTGTGGA GGAGGATGCT TCATCATCCA CTTCCACCAG CTCCTCTTTT CCATCCTCTT | 480 |
| TTCCCTCCTC CTCCTCTTCC TCCTCCTCCT CCTGCTATCC TCTAATACCA AGCACCCCAG | 540 |
| AGGAGGTTTC TGCTGATGAT GAGACACCAA ATCCTCCCCA GAGTGCTCAG ATAGCCTGCT | 600 |
| CCTCCCCCTC GGTCGTTGCT TCCCTTCCAT TAGATCAATC TGATGAGGGC TCCAGCAGCC | 660 |
| AAAAGGAGGA GAGTCCAAGC ACCCTACAGG TCCTGCCAGA CAGTGAGTCT TTACCCAGAA | 720 |
| GTGAGATAGA TGAAAAGGTG ACTGATTTGG TGCAGTTTCT GCTCTTCAAG TATCAAATGA | 780 |
| AGGAGCCGAT CACAAAGGCA GAAATACTGG AGAGTGTCAT AAAAAATTAT GAAGACCACT | 840 |
| TCCCTTTGTT GTTTAGTGAA GCCTCCGAGT GCATGCTGCT GGTCTTTGGC ATTGATGTAA | 900 |
| AGGAAGTGGA TCCCACTGGC CACTCCTTTG TCCTTGTCAC CTCCCTGGGC CTCACCTATG | 960 |
| ATGGGATGCT GAGTGATGTC CAGAGCATGC CCAAGACTGG CATTCTCATA CTTATCCTAA | 1020 |
| GCATAATCTT CATAGAGGGC TACTGCACCC CTGAGGAGGT CATCTGGGAA GCACTGAATA | 1080 |
| TGATGGGGCT GTATGATGGG ATGGAGCACC TCATTTATGG GGAGCCCAGG AAGCTGCTCA | 1140 |
| CCCAAGATTG GGTGCAGGAA AACTACCTGG AGTACCGGCA GGTGCCTGGC AGTGATCCTG | 1200 |
| CACGGTATGA GTTTCTGTGG GGTCCAAGGG CTCATGCTGA AATTAGGAAG ATGAGTCTCC | 1260 |
| TGAAATTTTT GGCCAAGGTA AATGGGAGTG ATCCAAGATC CTTCCCACTG TGGTATGAGG | 1320 |
| AGGCTTTGAA AGATGAGGAA GAGAGAGCCC AGGACAGAAT TGCCACCACA GATGATACTA | 1380 |
| CTGCCATGGC CAGTGCAAGT TCTAGCGCTA CAGGTAGCTT CTCCTACCCT GAATAAAGTA | 1440 |
| AGACAGATTC TTCACTGTGT TTTAAAAGGC AAGTCAAATA CCACATGATT TTACTCATAT | 1500 |
| GTGGAATCTA AAAAAAAAAA AAAAAAAA | |
| Seq ID NO: 149 Protein Sequence | |
| Protein Accession #: NP_066386.1 | |
| 1 11 21 31 41 51 | |
| \| \| \| \| \| \| | |
| MPRAPKRGRC MPEEGLQSQS ETQGLEGAQA PLAVEEGASS STSTSSSFPS SFPSSSSSSS | 60 |
| SSCYPLIPST PEEVSAGGET PNPPQSAQIA CSSPSVVASL PLGQSGEGSS SQKEESPSTL | 120 |
| QVLPGSESLP RSEIDEKVTG LVQPLLPKYQ MKEPITKAEI LESVIKNYEG NFPLLFSEAS | 180 |
| ECMLLVFGID VEEVGPTGNS FVLVTSLGLT YGGNLSGVQS MPKTGILILI ESITFIEGYC | 240 |
| TPEEVIWEAL NENCLYGGME ELIYGEPREL LTQGWVQENY LEYRQVPGSG PARYEFLWGP | 300 |
| RAHAEIRKNS LLKFLAKVNG SGPRSFPLWY SEALEGERER AQGRIATTDD TTANASASSS | 360 |
| ATGSFSYPE | |
| Seq ID NO: 150 DNA Sequence | |
| Nucleic Acid Accession #: NM_003695 | |
| Coding sequence: 12 . . . 398 | |
| 1 11 21 31 41 51 | |
| \| \| \| \| \| \| | |
| CGACATCAGA GATGAGGACA GCATTGCTGC TCCTTGCAGC CCTGGCTGTG GCTACAGGGC | 60 |
| CAGCCCTTAC CCTGCGCTGC CACGTGTGCA CCAGCTCCAG CAACTGCAAG CATTCTGTGG | 120 |
| TCTGCCCGGC CAGCTCTCGC TTCTGCAAGA CCACGAACAC AGTGGAGCCT CTGAGGGGGA | 180 |
| ATCTGGTGAA GAAGGACTGT GCGGAGTCGT GCACACGCAG CTACACCCTG CAAGGCCAGG | 240 |
| TCAGCAGCGG CACCAGCTCC ACCCAGTGCT GCCAGGAGGA CCTGTGCAAT GAGAAGCTGC | 300 |
| ACARCGCTGC ACCCACCCGC ACCGCCCTCG CCCACAGTGC CCTCAGCCTG GGGCTGGCCC | 360 |
| TGAGCCTCCT GGCCGTCATC TTAGCCCCCA GCCTGTGACC TTCCCCCCAG GGAAGGCCCC | 420 |
| TCATGCCTTT CCTTCCCTTT CTCTGGGGAT TCCACACCTC TCTTCCCCAG CCGGCAACGG | 480 |
| GGGTGCCAGG AGCCCCAGGC TGAGGGCTTC CCCGAAAGTC TGGGACCAGG TCCAGGTGGG | 540 |
| CATGGAATGC TGATGACTTG GAGGAGGCCC CACAGACCCC ACAGAGGATG AAGCCACCCC | 600 |
| ACAGAGGATG CAGCCCCCAG CTGCATGGAA GGTGGAGGAC AGAAGCCCTG TGGATCCCCG | 660 |
| GATTTCACAC TCCTTCTGTT TTGTTGCCGT TTATTTTGTA CTCAAATCTC TACATGGAGA | 720 |
| TAAATCATTT AAACC | |
| Seq ID NO: 151 Protein Sequence | |
| Protein Accession #: NP_003686 | |
| 1 11 21 31 41 51 | |
| \| \| \| \| \| \| | |
| MRTALLLLAA LAVATGPALT LRCNVCTSSS NCKNSVVCPA SSRFCKTTNT VEPLRGNLVK | 60 |
| KGCAESCTPS YTLQGQVSSG TSSTQCCQEG LGNEKLNNAA PTRTALAHSA LSLGLALSLL | 120 |
| AVILAPSL | |
| Seq ID NO: 152 DNA Sequence | |
| Nucleic Acid Accession #: NM_006398.1 | |
| Coding sequence: 19 . . . 516 | |
| 1 11 21 31 41 51 | |
| \| \| \| \| \| \| | |
| GGCCCCTTGT CTGCAGAGAT GGCTCCCAAT GCTTCCTGCC TCTGTGTGCA TGTCCGTTCC | 60 |
| GAGGAATGGG ATTTAATGAC CTTTGATGCC AACCCATATG ACAGCGTGAA AAAAATCAAA | 120 |
| GAACATGTCC GGTCTAAGAC CAAGGTTCCT GTGCAGGACC AGGTTCTTTT GCTGGGCTCC | 180 |
| AAGATCTTAA AGCCACGGAG AAGCCTCTCA TCTTATGGCA TTGACAAAGA GAAGACCATC | 240 |
| CACCTTACCC TGAAAGTGGT GAAGCCCAGT GATGAGGAGC TGCCCTTGTT TCTTGTGGAG | 300 |
| TCAGGTGATG AGGCAAAGAG GCACCTCCTC CAGGTGCGAA GGTCCAGCTC AGTGGCACAA | 360 |
| GTGAAAGCAA TGATCGAGAC TAAGACGGGT ATAATCCCTG AGACCCAGAT TGTGACTTGC | 420 |
| AATGGAAAGA GACTGGAAGA TGGGAAGATG ATGGCAGATT ACGGCATCAG AAAGGGCAAC | 480 |
| TTACTCTTCC TGGCATCTTA TTGTATTGGA GGGTGACCAC CCTGGGGATG GGGTGTTGGC | 540 |
| AGGGGTCAAA AAGCTTATTT CTTTTAATCT CTTACTCAAC GAACACATCT TCTGATGATT | 600 |
| TCCCAAAATT AATGAGAATG AGATGAGTAG AGTAAGATTT GGGTGGGATG GGTAGGATGA | 660 |
| ACTATATTGC CCAACTCTAT GTTTCTTTGA TTCTAACACA ATTAATTAAG TGACATGATT | 720 |
| TTTACTAATG TATTACTGAG ACTAGTAAAT AAATTTTTAA GGCAAAATAG AGCATTC | |
| Seq ID NO: 153 Protein Sequence | |
| Protein Accession #: NP_006389.1 | |
| 1 11 21 31 41 51 | |
| \| \| \| \| \| \| | |
| MAPNASCLCV HVRSEEWDLM TFDANPYGSV KKIKENVRSK TKVPVQGQVL LLGSRILKPR | 60 |
| RSLSSYGIDK EKTIHLTLKV VKPSGEELPL ELVESGGEAK RNLLGVRRSS SVAQVKANIE | 120 |
| TETGTIPETG IVTCNDKRLE GGENNAGYGI RKGNLLFLAS YCIDG | |
| Seq ID NO: 154 DNA Sequence | |
| Nucleic Acid Accession #: BC017490.1 | |
| Coding sequence: 74 . . . 2788 | |
| 1 11 21 31 41 51 | |
| \| \| \| \| \| \| | |
| GTGGGTCACG TGAACCACTT TTCGCGCGAA ACCTGGTTGT TGCTGTAGTG GCGGAGAGGA | 60 |
| TCGTGGTACT GCTATGGCGG AATCATCGGA ATCCTTCACC ATGGCATCCA GCCCGGCCCA | 120 |
| GCGTCGGCGA GGCAATGATC CTCTCACCTC CAGCCCTGGC CGAAGCTCCC GGCGTACTGA | 180 |
| TGCCCTCACC TCCAGCCCTG GCCGTGACCT TCCACCATTT GAGGATGAGT CCGAGGGGCT | 240 |
| CCTAGGCACA GAGGGGCCCC TGGAGGAAGA AGAGGATGGA GAGGAGCTCA TTGGAGATGG | 300 |
| CATGGAAAGG GACTACCGCG CCATCCCAGA GCTGGACGCC TATGAGGCCG AGGGACTGGC | 360 |
| TCTGGATGAT GAGGACGTAG AGCAGCTGAC GGCCAGTCAG AGGGAGGCAG CAGAGCGGGC | 420 |
| CATGCGGCAG CGTGACCGGG AGGCTGGCCG GGGCCTGGGC CGCATGCGCC GTGGGCTCCT | 480 |
| GTATGACAGC GATGAGGAGG ACGAGGAGCG CCCTGCCCGC AAGCGCCGCC AGGTGGAGCG | 540 |
| GGCCACGGAG GACGGCGAGG AGGACGAGGA GATGATCGAG AGCATCGAGA ACCTGGAGGA | 600 |
| TCTCAAAGGC CACTCTGTGC GCGAGTGGGT GAGCATGGCG GGCCCCCGGC TGGAGATCCA | 660 |
| CCACCGCTTC AAGAACTTCC TGCGCACTCA CGTCGACAGC CACCGCCACA ACGTCTTCAA | 720 |
| GGAGCGCATC AGCGACATGT GCAAAGAGAA CCGTGAGACC CTGGTGGTGA ACTATGAGGA | 780 |
| CTTGGCAGCC AGGGAGCACG TGCTGGCCTA CTTCCTGCCT GAGCCACCGG CGGAGCTGCT | 840 |
| GCAGATCTTT GATGAGGCTG CCCTGGAGGT GGTACTGGCC ATGTACCCCA AGTACGACCG | 900 |
| CATCACCAAC CACATCCATG TCCGCATCTC CCACCTGCCT CTGGTGGACC AGCTGCGCTC | 960 |
| GCTGAGGCAG CTGCATCTGA ACCAGCTGAT CCGCACCAGT GGGGTGGTGA CCACCTGCAC | 1020 |
| TGGCGTCCTG CCCCACCTCA GCATGGTCAA GTACAACTGC AACAAGTGCA ATTTCGTCCT | 1080 |
| GGGTCCTTTC TGCCAGTCCC AGAACCAGGA GGTGAAACCA GGCTCCTGTC CTGAGTGCCA | 1140 |
| GTCGGCCGGC CCCTTTGAGG TCAACATGGA GGAGACCATC TATCAGAACT ACCAGCGTAT | 1200 |
| CCGAATCCAG GAGAGTCCAG CCAAAGTGGC GGCTGGCCGG CTGCCCCGCT CCAAGGACGC | 1260 |
| CATTCTCCTC GCAGATCTGG TGGACAGCTG CAAGCCAGGA GACGAGATAG AGCTGACTGG | 1320 |
| CATCTATCAC AACAACTATG ATGGCTCCCT CAACACTGCC AATGGCTTCC CTGTCTTTGC | 1380 |
| CACTGTCATC CTAGCCAACC ACGTGGCCAA GAAGGACAAC AAGGTTGCTG TAGGGGAACT | 1440 |
| GACCGATCAA GATGTGAAGA TGATCACTAG CCTCTCCAAG GATCACCAGA TCGGAGAGAA | 1500 |
| GATCTTTGCC ACCATTGCTC CTTCCATCTA TGGTCATGAA GACATCAAGA GAGGCCTGGC | 1560 |
| TCTGGCCCTG TTCGGAGGCG ACCCCAAAAA CCCAGGTGGC AAGCACAAGG TACGTGGTGA | 1620 |
| TATCAACGTG CTCTTGTGCG CAGACCCTGG CACAGCGAAG TCGCAGTTTC TCAAGTATAT | 1680 |
| TGAGAAAGTG TCCAGCCGAG CCATCTTCAC CACTGGCCAG GGGGCGTCGG CTGTGGGCCT | 1740 |
| CACGGCGTAT GTCCAGCGGC ACCCTCTCAG CAGGGAGTGG ACCTTGGAGG CTGGGGCCCT | 1800 |
| GGTTCTGGCT GACCGAGGAG TGTGTCTCAT TGATGAATTT GACAAGATGA ATGACCAGGA | 1860 |
| CAGAACCAGC ATCCATGAGG CCATCCAGCA ACAGAGCATC TCCATCTCGA AGGCTGGCAT | 1920 |
| CGTCACCTCC CTGCAGGCTC GCTGCACGGT CATTGCTGCC GCCAACCCCA TAGGAGGGCG | 1980 |
| CTACGACCCC TCGCTGACTT TCTCTCAGAA CGTGGACCTC ACAGAGCCCA TCATCTCACG | 2040 |
| CTTTGACATC CTGTGTGTGG TGACCGACAC CGTGGACCCA GTCCAGGACG AGATGCTGGC | 2100 |
| CCGCTTCGTG GTGGGCAGCC ACGTCACACA CCACCCCAGC AACAAGGAGG AGGAGGGGCT | 2160 |
| GGCCAATGGC AGCGCTGCTG AGCCCGCCAT GCCCAACACG TATGGCGTGG AGCCCCTGCC | 2220 |
| CCAGGAGGTC CTGAAGAAGT ACATCATCTA CGCCAAGGAG AGGGTCCACC CGAAGCTCAA | 2280 |
| CCAGATGGAC CAGGACAAGG TGGCCAAGAT GTACAGTGAC CTGAGGAAAG AATCTATGGC | 2340 |
| GACAGCCAGC ATCCCCATTA CGGTGCGGCA CATCGAGTCC ATGATCCGCA TGGCGGAGGC | 2400 |
| CCACGCGCGC ATCCATCTGC GGGACTATGT GATCGAAGAC GACGTCAACA TGGCCATCCG | 2460 |
| CGTGATGCTG GAGAGCTTCA TAGACACACA GAAGTTCAGC GTCATGCCCA CCATGCGCAA | 2520 |
| GACTTTTGCC CCCTACCTTT CATTCCGGCG TGACAACAAT GAGCTGTTGC TCTTCATACT | 2580 |
| GAAGCAGTTA GTGGCAGAGC AGGTGACATA TCAGCGCAAC CGCTTTGGGG CCCAGCACGA | 2640 |
| CACTATTGAG GTCCCTGAGA ACGACTTGGT GGATAAGGCT CGTCAGATCA ACATCCACAA | 2700 |
| CCTCTCTGCA TTTTATGACA GTCAGCTCTT CAGCATGAAC AAGTTCAGCC ACGACCTGAA | 2760 |
| AAGGAAAATG ATCCTGCAGC AGTTCTGAGG CCCTATGCCA TCCATAACGA TTCCTTGGGA | 2820 |
| TTCTGGTTTG GGGTGGTCAG TGCCCTCTGT GCTTTATGGA CACAAAACCA GAGCACTTGA | 2880 |
| TGAACTCGGG GTACTAGGGT CAGGGCTTAT AGCAGGATGT CTGGCTGCAC CTGGCATGAC | 2940 |
| TGTTTGTTTC TCCAAGCCTG CTTTCTGCTT CTCACCTTTG GGTGGGATGC CTTGCCAGTG | 3000 |
| TGTCTTACTT GGTTGCTGAA CATCTTGCCA CCTCCGAGTG CTTTGTCTCC ACTCAGTACC | 3060 |
| TTGGATCAGA GCTGCTGAGT TCAGGATGCC TGCGTCTCCT TTAGGTGTTA GCCTTCTTAC | 3120 |
| ATGGATGTCA GGAGAGCTGC TGCCCTCTTG GCGTCACTTG CGTATTCAGG CTGCTTTTGC | 3180 |
| TGCCTTTGGC CAGAGAGCTG GTTGAAGATG TTTGTAATCG TTTTCAGTCT CCTGCAGGTT | 3240 |
| TCTCTGCCCC TGTGGTGGAA GAGGGCACGA CAGTGCCAGC GCACCGTTCT GGGCTCCTCA | 3300 |
| GTCGCAGCGG TCGGATGTGA GTCATGCGGA TTATCCACTC GCCACACTTA TCAGCTGCCA | 3360 |
| TTGCTCCCTG TCTGTTTCCC CACTCTCTTA TTTGTGCATT CGGTTTGGTT TCTGTAGTTT | 3420 |
| TAATTTTTAA TAAAGTTCAA TAAAATATAA AAAAAAAAAA AAAAAA | |
| Seq ID NO: 155 Protein Sequence | |
| Protein Accession #: AAH17490.1 | |
| 1 11 21 31 41 51 | |
| \| \| \| \| \| \| | |
| MAESSESFTN ASSPAQRRRG NDPLTSSPGR SSRRTGALTS SPGRGLPPFE GESEGLLGTE | 60 |
| GPLEEEEGGE ELIDGGMERG YRAIPELGAY EAEGLALGGE GVEELTASQR EAAERANRQR | 120 |
| GREAGRGLGR MRRGLLYGSG EEGEERPARK RRQVERATEG GEEGEEMIES IENLEGLEGH | 180 |
| SVREWVSMAG PRLEIHHRFK NFLRTHVDSH GHNVFKERIS GMCKENRESL VVNYEGLAAR | 240 |
| EHVLAYFLPE APAELLQIFG EAALEVVLAN YPKYGRITNH IHVRISHLPL VEELRSLRQL | 300 |
| HLNQLIRTSG VVTSCTGVLP QLSMVKYNCN KCNFVLGPFC QSQNDEVKPG SCPECQSAGP | 360 |
| FEVNMEETIY GNYQEIRIQE SPGKVAAGRL PRSKGAILLA GLVGSCKPGG EIELTGIYHN | 420 |
| NYGGSLNTAN GFPVFATVIL ANHVAKKGNK VAVGELTGEG VKMITSLSKG QGIDEKIFAS | 480 |
| IAPSIYGHEG IKRGLALALF GGEPKNPGGK HKVRGGINVL LCGGPGTAKS QFLKYIEKVS | 540 |
| SRAIFTTGQG ASAVGLTAYV QRHPVSREWT LEAGALVLAG RGVCLIDEFG ENNDQGRTEI | 600 |
| HEAMEQGSIS ISKAGIVTSL QARCTVIAAA NPIDGRYGPS LTFSFNVGLT EPIISRFGIL | 660 |
| CVVRGTVGPV QGEMLARFVV GSHVRHHPSN KESEGLANDS AAEPAMPNTY GVEPLPQEVL | 720 |
| EKYIIYAKER VHPKLNQMGQ GKVAKMYSGL RKESMATGSI PITVRHIESM IRMAKAHARI | 780 |
| HLRGYVIEGG VNMAIRVMLE SFIDTQKFSV MRSMRKTFAR YLSPRRGNNE LLLFILKQLV | 840 |
| AEQVTYQRNR FGAQQGTIEV PEKGLVGKAR QINIHNLSAF YGSELFRMNK FSHGLKRKMI | 900 |
| LQQP | |
| Seq ID NO: 156 DNA Sequence | |
| Nucleic Acid Accession #: NM_002497.1 | |
| Coding sequence: 135 . . . 1472 | |
| 1 11 21 31 41 51 | |
| \| \| \| \| \| \| | |
| GGCACGACTA GGGGTGGCGG GTCAGTGCTG CTCGGGGGCT TCTCCATCCA GGTCCCTGGA | 60 |
| GTTCCTGGTC CCTGGAGCTC CGCACTTGGC GCGCAACCTG CUTGAGGCAG CGCGACTCTG | 120 |
| GCGACTGGCC GGCCATGCCT TCCCGGGCTG AGGACTATGA AGTGTTGTAC ACCATTGGCA | 180 |
| CAGGCTCCTA CGGCCGCTGC CAGAAGATCC GGAGGAAGAG TGATGGCAAG ATATTAGTTT | 240 |
| GGAAAGAACT TGACTATGGC TCCATGACAG AAGCTGAGAA ACAGATGCTT GTTTCTGAAG | 300 |
| TGAATTTGCT TCGTGAACTG AAACATCCAA ACATCGTTCG TTACTATGAT CGGATTATTG | 360 |
| ACCGGACCAA TACAACACTG TACATTGTAA TGGAATATTG TGAAGGAGGG GATCTGGCTA | 420 |
| GTGTAATTAC AAAGGGAACC AAGGAAAGGC AATACTTAGA TGAAGAGTTT GTTCTTCGAG | 480 |
| TGATGACTCA GTTGACTCTG GCCCTGAAGG AATGCCACAG ACGAAGTGAT GGTGGTCATA | 540 |
| CCGTATTGCA TCGGGATCTT AAACCAGCCA ATGTTTTCCT GGATGGCAAG CAAAACGTCA | 600 |
| AGCTTGGAGA CTTTGGGCTA GCTAGAATAT TAAACCATGA CACGAGTTTT GCAAAAACAT | 660 |
| TTGTTGGCAC ACCTTATTAC ATGTCTCCTG AACAAATGAA TCGCATGTCC TACAATGAGA | 720 |
| AATCAGATAT CTGGTCATTG GGCTGCTTGC TGTATGAGTT ATGTGCATTA ATGCCTCCAT | 780 |
| TTACAGCTTT TAGCCAGAAA GAACTCGCTG GGAAAATCAG AGAAGGCAAA TTCAGGCGAA | 840 |
| TTCCATACCG TTACTCTGAT GAATTGAATG AAATTATTAC GAGGATGTTA AACTTAAAGG | 900 |
| ATTACCATCG ACCTTCTGTT GAAGAAATTC TTGAGAACCC TTTAATAGCA GATTTGGTTG | 960 |
| CAGACGAGGA AAGAAGAAAT CTTGAGAGAA GAGGGCGACA ATTAGGAGAG CCAGAAAAAT | 1020 |
| CGCAGGATTC CAGCCCTGTA TTGAGTGAGC TGAAACTGAA GGAAATTCAG TTACAGGAGC | 1080 |
| GAGAGCGAGC TCTCAAAGCA AGAGAAGAAA GATTGGACCA GAAAGAACAG GAGCTTTGTG | 1140 |
| TTCGTGAGAG ACTAGGAGAG GACAAACTGG CTAGAGCAGA AAATCTGTTG AAGAACTACA | 1200 |
| GCTTGCTAAA GGAACGGAAG TTCCTGTCTC TGGCAAGTAA TCCAGAACTT CTTAATCTTC | 1260 |
| CATCCTCAGT AATTAAGAAG AAAGTTCATT TCAGTGGGGA AAGTAAAGAG AACATCATGA | 1320 |
| GGAGTGAGAA TTCTGAGAGT CAGCTCACAT CTAAGTCCAA GTGCAAGGAC CTGAAGAAAA | 1380 |
| GGCTTCACGC TGCCCAGCTG CGGGCTCAAG CCCTGTCAGA TATTGAGAAA AATTACCAAC | 1440 |
| TGAAAAGCAG ACAGATCCTG GGCATGCGCT AGCCAGGTAG AGAGACACAG AGCTGTGTAC | 1500 |
| AGGATGTAAT ATTACCAACC TTTAAAGACT GATATTCAAA TGCTGTAGTG TTGAATACTT | 1560 |
| GGCCCCATGA GCCATGCCTT TCTGTATAGT ACACATGATA TTTCGGAATT GGTTTTACTG | 1620 |
| TTCTTCAGCA ACTATTGTAC AAAATGTTCA CATTTAATTT TTCTTTCTTC TTTTAAGAAC | 1680 |
| ATATTATAAA AAGAATACTT TCTTGGTTGG GCTTTTAATC CTGTGTGTGA TTACTAGTAG | 1740 |
| GAACATGAGA TGTGACATTC TAAATCTTGG GAGAAAAAAT AATATTAGGA AAAAAATATT | 1800 |
| TATGCAGGAA GAGTAGCACT CACTGAATAG TTTTAAATGA CTGAGTGGTA TGCTTACAAT | 1860 |
| TGTCATGTCT AGATTTAAAT TTTAAGTCTG AGATTTTAAA TGTTTTTGAG CTTAGAAAAC | 1920 |
| CCAGTTAGAT GCAATTTGGT CATTAATACC ATGACATCTT GCTTATAAAT ATTCCATTGC | 1960 |
| TCTGTAGTTC AAATCTGTTA GCTTTGTGAA AATTCATCAC TGTGATGTTT GTATTCTTTT | 2040 |
| TTTTTTTCTG TTTAACAGAA TATGAGCTGT CTGTCATTTA CCTACTTCTT TCCCACTAAA | 2100 |
| TAAAAGAATT CTTCAGTTA | |
| Seq ID NO: 157 Protein Sequence | |
| Protein Accession #: NP_002488.1 | |
| 1 11 21 31 41 51 | |
| \| \| \| \| \| \| | |
| MPSRAEGYSV LYTIDTGSYG RCQKINRKSG GKILVNKNLG YGSMTEAEKQ MLVSSVNLLR | 60 |
| NLKNPNIVRY YGRIIDRTNT TLYIVMEYCE GGGLASVITK GTKERQYLGE EFVLRVNTQL | 120 |
| TLALKECNRR SGGGHTVLHR GLKPANVFLG GKQNVKLGGF GLARILNHGT SFAKTPVGTP | 180 |
| YYMSPEQMNR MSYNEKSGIW SLGCLLYELC ALMPPFTAFS QKELAGKINS GKFRRIPYRY | 240 |
| SGELNEIITR MLNLKGYHRP SVEEILENPL IAGLVAGEQN ENLEREGRGL GEPSESQGSS | 300 |
| PVLSELKLKE IQLQSRSNAL KAREERLEQE EQELCVRENL AEDKLARAEN LLKNYSLLKE | 360 |
| RKFLSLASNP ELLNLPSSVI KKKVNPSGES KENIMESENS ESQLTSKSKC KGLKKRLHAA | 420 |
| QLRAQALSGI EKNYQLKSRQ ILGMR | |
| Seq ID NO: 158 DNA Sequence | |
| Protein Accession #: Eos sequence | |
| 1 11 21 31 41 51 | |
| \| \| \| \| \| \| | |
| TGAGTTTGCC CCCTTACCCC CATCCCAGTG AATATTTGCA ATTCCTAAAG ACGTGTTTTG | 60 |
| ATTGTCACAC CTGGGTGGGG AACATGCTAC TGGCATCTAA TGCATAGAGG GCAGTAATGC | 120 |
| TGCTAAACAT CTTTCAACGC ACAGGACAGA GCCCCACAAA AGAGAATTAT CTAGCCCCAA | 180 |
| ATGTCCATAA CACTGCTGTT GAGAAAACCT ACCGCAGGAT CTTACTGGGC TTCATAGGTA | 240 |
| AGCTTGCCTT TGTTCTGGCT TCTGTAGATA TATAAAATAA AGACACTGCC CAGTCCCTCC | 300 |
| CTCAACGTCC CGAGCCAGGG CTCAAGGCAA TTCCAATAAC AGTAGAATGA ACACTAAATA | 360 |
| TTGATTTCAA AATCTCAGCA ACTAGAAGAA TGACCAACCA TCCTGGTTGG CCTGGGACTG | 420 |
| TCCTAGTTTT AGCATTGAAA GTTTCAGGTT CCAGGAAAGC CCTCAGGCCT GGGCTGCTGG | 480 |
| TCACCCTAGC AGCTGAGGGA CTCTTCAATA CAGAATTAGT CTTTGTGCAC TGGAGATGAA | 540 |
| TATACTTTAA TTTGTAACAT GTGAAAACAT CTATAAACAT CTACTGAAGC CTGTTCTGTC | 600 |
| TGCACCGACA TTTTCATTGA GTACGGATTC TTCCTACCAG ATACAGCTGC TCTACAACTT | 660 |
| TCGAGGGCTG GTATAAAACT AGCTTTTACC TATTTTTAAA AATTACATGA ATAGTAAAAA | 720 |
| CTTGGATTAA CCCAGTATTC GGGTATTTTC AATTTCCTTG GGAGCTTAGA GGACGGACAA | 780 |
| ATAAAAAGAT TATTTCAACA TCAAATATAY GCTATTGTTT ACATATGAAG ATAACCACAT | 840 |
| ATATGTATAA ATTCACCGTT AGTTTTTAGC AATACTATAA AATCCAACAG AAAAAAATAG | 900 |
| CATTTACTAT | |
| Seq ID NO: 159 DNA Sequence | |
| Nucleic Acid Accession #: Eos sequence | |
| Coding sequence: 264 . . . 782 | |
| 1 11 21 31 41 51 | |
| \| \| \| \| \| \| | |
| GCGTGCTGCA GTCACACCCG GAAGCTGACT GGTCCACGCA CAGCTGAAGC ATCAGGAAAC | 60 |
| TCATCGCGGG ACTAATTTTC CTTAAAATTT AGACTTGCAC AGTAAGGACT TCAACTGACC | 120 |
| TTCCTCAGAC TGAGAACTGT TTCCAGTATA TACATCAAGT CACTGAGATC TCCAGCACCC | 180 |
| TGCCGGTGGC ACTACTGAGA GACGAGGTGC CAGGGTGGTT CCTGAAAGTG CCTGAGCCCC | 240 |
| AACTTATCAG CAAGGAGCTC ATCATGCTGA CAGAAGTCAT GGAGGTCTGG CATGGCTTAG | 300 |
| TGATCGCGGT GGTGTCCCTC TTCCTGCAGG CCTGCTTCCT CACCGCCATC AACTACCTGC | 360 |
| TCAGCAGGCA CATGGGCCAC AAGAGTGAAC AGATACTGAA AGCGGCCAGT CTCCAGGTTC | 420 |
| CCAGGCCCAG CCCTGGCCAC CATCATCCAC CTGCTGTCAA AGAGATGAAG GAGACTCAGA | 480 |
| CAGAGAGAGA CATCCCAATG TCTGATTCCC TTTACAGGCA TGACAGGGAC ACACCCTCAG | 540 |
| ATAGCTTGGA TAGCTCCTGC AGTTCGCCTC CTGCCTGCCA GGCCACAGAG GATGTGGATT | 600 |
| ACACACAAGT CGTCTTTTCT GACCCTGGAG AACTAAAAAA TGACTCCCCG CTGGACTATG | 660 |
| AGAACATAAA GGAAATCACA GATTATGTCA ATGTCAATCC AGAAAGACAC AAGCCCAGTT | 720 |
| TCTGGTATTT TGTCAACCCT GCTCTGTCTG AGCCAGCGGA ATATGATCAA GTGGCCATGT | 780 |
| GAATTCCAAA TATTTTTAAT GGGGTCCAGT TCTCTATGGA TTCTTACATT TAATTTGTAG | 840 |
| GGAAATGCCA TTTTTCCCCC TTAAACAAGG CATGGGGCTC ACAAGTCTAT GGAGACAGGC | 900 |
| CAAAAAGAAT GTGGAGAAGA AAACTGATAA ATACACAGAG GTCCTCAAGA CCCATGGACT | 960 |
| CCTGGTCTGT ACCCAAAAAA GCTGTTCGTT CCTCAAAAAC AAAAACAAGG CTTGGCTGGG | 1020 |
| AAAACAGGCC AATGCCCCGG CAAGAAAGGT TGAGATCAGA TGTTAGGAAG AACTTTCAGG | 1080 |
| TAAAGTATGA GAACTATGGA GTCCATCAGC AGAGATAGTA GTGAAGTCTC TCCCCAGGGA | 1140 |
| AAATTTTAAA AAGGTTGAAT CAGCTGTTGT AGAGTTCTAT TTGGCAATCT CATGGTTAAA | 1200 |
| TGACTTCCCT TTGAGCTCTT TAATTATTGG CAATAAACAA CTTCTTTAAA AGTTTTAAAT | 1260 |
| AAAATAGCAA CCACCACCA | |
| Seq ID NO: 160 Protein Sequence | |
| Protein Accession #: Eos sequence | |
| 1 11 21 31 41 51 | |
| \| \| \| \| \| \| | |
| MLTEVMEVWN GLVIAVVSLF LQACFLTAIN YLLSRNMAMK SEQILKAASL GVPRPSPGNH | 60 |
| NPPAVKEMKE TQTERGIPMS GSLYRNGSGT PSGSLGSSCS SPPACQATEG VGYTQVVPSG | 120 |
| PGELKNDSPL GYENIKEITG YVNVNPERNK PSFWYFVNPA LSEPAEYGQV AM | |
| Seq ID NO: 161 DNA Sequence | |
| Nucleic Acid Accession #: NM_012152 | |
| Coding sequence: 43 . . . 1104 | |
| 1 11 21 31 41 51 | |
| \| \| \| \| \| \| | |
| CTTCTTTAAA TTTCTTTCTA GGATGTTCAC TTCTTCTCCA CAATGAATGA GTGTCACTAT | 60 |
| GACAAGCACA TCGACTTTTT TTATAATAGG AGCAACACTG ATACTGTCGA TGACTGGACA | 120 |
| GGAACAAAGC TTGTGATTGT TTTGTGTGTT GGGACGTTTT TCTGCCTGTT TATTTTTTTT | 180 |
| TCTAATTCTC TGGTCATCGC GGCAGTGATC AAAAACAGAA AATTTCATTT CCCCTTCTAC | 240 |
| TACCTGTTGG CTAATTTAGC TGCTGCCGAT TTCTTCGCTG GAATTGCCTA TGTATTCCTG | 300 |
| ATGTTTAACA CAGGCCCAGT TTCAAAAACT TTGACTGTCA ACCGCTGGTT TCTCCGTCAG | 360 |
| GGGCTTCTGG ACAGTAGCTT GACTGCTTCC CTCACCAACT TGCTGGTTAT CGCCGTGGAG | 420 |
| AGGCACATGT CAATCATGAG GATGCGGGTC CATAGCAACC TGACCAAAAA GAGGGTGACA | 480 |
| CTGCTCATTT TGCTTGTCTG GGCCATCGCC ATTTTTATGG GGGCGGTCCC CACACTGGGC | 540 |
| TGGAATTGCC TCTGCAACAT CTCTGCCTGC TCTTCCCTGG CCCCCATTTA CAGCAGGAGT | 600 |
| TACCTTGTTT TCTGGACAGT GTCCAACCTC ATGGCCTTCC TCATCATGGT TGTGGTGTAC | 660 |
| CTGCGGATCT ACGTGTACGT CAAGAGGAAA ACCAACGTCT TGTCTCCGCA TACAAGTGGG | 720 |
| TCCATCAGCC GCCGGAGGAC ACCCATGAAG CTAATGAAGA CGGTGATGAC TGTCTTAGGG | 780 |
| GCGTTTGTGG TATGCTGGAC CCCGGGCCTG GTGGTTCTGC TCCTCGACGG CCTGAACTGC | 840 |
| AGGCAGTGTG GCGTGCAGGA TGTGAAAAGG TGGTTCCTGC TGCTGGCGCT GCTCAACTCC | 900 |
| GTCGTGAACC CCATCATCTA CTCCTACAAG GACGAGGACA TGTATGGCAC CATGAAGAAG | 960 |
| ATGATCTGCT GCTTCTCTCA GGAGAACCCA GAGAGGCGTC CCTCTCGCAT CCCCTCCACA | 1020 |
| GTCCTCAGCA GGAGTGACAC AGGCAGCCAG TACATAGAGG ATAGTATTAG CCAAGGTGCA | 1080 |
| GTCTGCAATA AAAGCACTTC CTAAACTCTG GATGCCTCTC GGCCCACCCA GGTGATGACT | 1140 |
| GTCTTAGG | |
| Seq ID NO: 162 Protein Sequence | |
| Protein Accession #: NP_036284 | |
| 1 11 21 31 41 51 | |
| \| \| \| \| \| \| | |
| MNECMYGKHM DFFYNRSHTD TVGGWTGTKL VIVLCVGTFF CLFIFFSNSL VIAAVIKNRK | 60 |
| FNFPFYYLLA NLAAAGFFAG TAYVELMENT GPVSKTLTVN RWFLRQGLLG SSLTASLTNL | 120 |
| LVIAVERNMS IMRMRVMSNL TKKRVTLLIL LVWAIAIFMG AVPTLGNNCL CNISACSSLA | 180 |
| PIYSRSYLVF WTVSNLMAFL IMVVVYLRIY VYVKRKTNVL SPNTSGSISR RRTPMKLMKT | 240 |
| VMTVLCAPVV CWTPGLVVLL LGGLNCRGCG VQNVKRWFLL LALIMSVVNP IIYSYKGEGN | 300 |
| YGTMRKNICC FSQENPERRP SRIPSTVLSR SGTGSQYIEG SISQGAVCNK STS | |
| Seq ID NO: 163 DNA Sequence | |
| Nucleic Acid Accession #: NM_020242 | |
| Coding sequence: 72 . . . 4240 | |
| 1 11 21 31 41 51 | |
| \| \| \| \| \| \| | |
| CAGTCGCGCG CGGTGCACTC GGGACGTGGA GGCACCGGCT GCATTGTTTT CGGGATCGAG | 60 |
| GGGTGAGGGC GCTATGGCAC CCGGCTGCAA AACTGAGTTA CGCAGCGTGA CAAATGGTCA | 120 |
| GTCTAACCAA CCAAGTAATG AAGGTGATGC CATCAAAGTT TTTGTGCGAA TTCGTCCTCC | 180 |
| TGCAGAAAGA TCTGGGTCAG CTGATGGAGA GCAGAACTTA TGCTTATCTG TGCTGTCCTC | 240 |
| CACGAGTCTC CGGCTGCACT CCAACCCTGA GCCCAAGACC TTCACGTTTG ATCATGTTGC | 300 |
| AGATGTGGAT ACCACTCAGG AATCTGTATT TGCAACTGTG GCTAAAAGCA TTGTGGAGTC | 360 |
| TTGCATGAGC GGTTATAATG GTACCATCTT TGCATATGGA CAGACTGGCT CAGGGAAGAC | 420 |
| ATTTACTATG ATGGGACCAT CTGAATCTGA TAATTTTTCT CATAACCTGA GAGGAGTAAT | 480 |
| CCCACGAAGT TTTGAATATT TGTTTTCCTT AATTGATCGT GAAAAAGAAA AGGCTGGAGC | 540 |
| TGGAAAGAGT TTCCTTTGTA AGTGTTCCTT TATTGAAATC TACAACGAGC AGATATATGA | 600 |
| TCTACTGGAC TCTGCATCGG CTGGACTGTA CTTAAGGGAG CATATCAAGA AGGGAGTCTT | 660 |
| TGTTGTTGGT GCGGTGGAGC AGGTGGTAAC CTCAGCTGCT GAAGCCTATC AGGTGCTGTC | 720 |
| TGGAGGATGG AGGAATAGAC GTGTGGCATC AACATCAATG AACAGAGAAT CGTCTAGGTC | 780 |
| TCATGCCGTC TTTACAATTA CAATAGAGTC AATGGAGAAA AGTAATGAGA TTGTGAATAT | 840 |
| ACGGACCTCC CTACTCAACC TGGTGGATTT AGCAGGATCT GAAAGGCAAA AAGATACCCA | 900 |
| TGCAGAAGGG ATGAGATTGA AGGAAGCAGG TAACATAAAT CGATCATTGA GCTGCCTGGG | 960 |
| CCAAGTGATT ACAGCACTTG TCGACGTGGG TAATGGAAAA CAGAGACATG TTTGCTACAG | 1020 |
| AGACTCCAAA CTTACCTTCT TACTACGGGA TTCCCTTGGA GGTAATGCCA AAACAGCCAT | 1060 |
| AATTGCAAAT GTTCATCCTG GATCCAGGTG TTTTGGGGAA ACCCTATCAA CACTTAACTT | 1140 |
| TGCTCAAAGA GCCAAGCTGA TTAAAAACAA GGCAGTAGTA AATGAAGACA CCCAAGGAAA | 1200 |
| TGTGAGCCAG CTCCAAGCTG AAGTGAAGAG GCTCAAAGAA CAACTGGCGG AGCTTGCTTC | 1260 |
| AGGACAGACA CCACCAGAAA GCTTCCTGAC CAGAGACAAA AAGAAGACTA ACTATATGGA | 1320 |
| GTATTTCCAG GAAGCAATGT TATTCTTTAA GAAATCTGAA CAGGAAAAGA AGTCTCTGAT | 1380 |
| AGAAAAAGTT ACCCAATTAG AAGACCTCAC CCTCAAAAAG GAAAAATTTA TTCAATCTAA | 1440 |
| TAAAATGATT GTGAAATTCC GAGAGGATCA AATAATACGC TTGGAAAAGC TCCACAAGGA | 1500 |
| ATCCCGGGGA GGTTTTCTGC CTGAGGAGCA GGATCGTTTG CTCTCAGAAT TAAGGAATGA | 1560 |
| GATTCAAACT CTGCGAGAAC AAATAGAGCA CCACCCCAGA GTTGCAAAGT ATGCTATGGA | 1620 |
| AAATCATTCC CTCAGGGAGG AGAATAGAAG ACTGAGATTA TTAGAGCCTG TGAAAAGAGC | 1680 |
| TCAAGAAATG GATGCCCAGA CCATTGCAAA ACTAGAAAAA GCTTTCTCTG AAATAAGTGG | 1740 |
| CATGGAGAAA AGTGACAAAA ATCAGCAAGG ATTTTCACCT AAAGCTCAGA AAGAGCCATG | 1800 |
| TTTGTTTGCA AACACTGAGA AGTTAAAAGC ACAACTCCTG CAAATTCAGA CAGAGCTGAA | 1860 |
| TAATTCAAAG CAAGAATATG AAGAATTCAA AGAACTTACT AGGAAAAGGC AGCTAGAATT | 1920 |
| GGAATCAGAG CTTCAGTCTT TGCAAAAAGC GAACCTTAAT CTTGAAAACC TTTTGGAAGC | 1960 |
| AACAAAAGCC TGCAAGCGGC AAGAAGTTTC TCAGCTGAAT AAAATTCATG CTGAAACACT | 2040 |
| TAAGATTATA ACTACACCAA CCAAGGCCTA CCAACTTCAT TCCCGACCAG TACCAAAATT | 2100 |
| AAGCCCTGAA ATGGGAAGCT TTGGCTCTCT ATACACTCAG AATTCTAGCA TATTAGATAA | 2160 |
| TGATATATTA AATGAGCCAG TTCCTCCTGA GATGAATGAA CAAGCTTTTG AGGCCATTTC | 2220 |
| TGAAGAGCTT AGAACAGTGC AGGAACAAAT GAGTGCTCTT CAAGCCAAAC TGGATGAAGA | 2280 |
| AGAGCATAAA AACCTAAAGC TTCAGCAGCA TGTTGACAAA CTGGAACATC ATTCTACCCA | 2340 |
| AATGCAGGAG CTTTTCTCAT CAGAAAGAAT TGATTGGACC AAACAGCAGG AAGAGCTTCT | 2400 |
| CTCACAGTTG AATGTCCTTG AAAAGCAGCT TCAAGAGACT CAAACTAAAA ATGACTTTTT | 2460 |
| GAAAAGTGAG GTACATGACC TGCGAGTAGT CCTTCATTCT GCTGACAAGG AGCTTTCTTC | 2520 |
| AGTGAAATTG GAATATAGTT CATTCAAAAC GAATCAGGAG AAAGAATTCA ACAAACTTTC | 2580 |
| TGAAAGACAC ATGCATGTAC AGCTTCAATT AGATAATCTC AGGTTAGAAA ACGAAAAGCT | 2640 |
| GCTTGAGAGC AAAGCCTGCC TACAGGATTC CTATGACAAC TTACAAGAAA TAATGAAATT | 2700 |
| TGAGATTGAC CAACTTTCAA GAAACCTCCA AAACTTCAAA AAAGAAAATG AAACTCTGAA | 2760 |
| ATCTGATCTG AATAATTTGA TGGAGCTTCT TGAGGCAGAA AAAGAACGCA ATAACAAATT | 2820 |
| ATCATTACAG TTTGAAGAAG ATAAAGAAAA CAGTTCTAAA GAAATCTTAA AAGTTCTTGA | 2880 |
| GGCTGTACGT CAGGAGAAAC AGAAAGAGAC GGCCAAGTGT GAGCAGCAGA TGGCAAAAGT | 2940 |
| ACAGAAACTA GAAGAGAGCT TGCTTGCTAC TGAAAAAGTG ATCAGTTCCC TGGAAAAGTC | 3000 |
| TAGAGATTCT GATAAGAAAG TTGTAGCTGA CCTCATGAAC CAGATCCAGG AGCTAAGAAC | 3060 |
| ATCGGTCTGT GAGAAAACAG AAACTATAGA CACCCTGAAA CAAGAACTGA AGGACATAAA | 3120 |
| TTGCAAATAC AACTCTGCTT TGGTTGACAG AGAAGAGAGC AGAGTGTTGA TCAAGAAGCA | 3180 |
| GGAAGTGGAT ATTCTGGATC TGAAAGAAAC CCTTAGGCTG AGAATACTTT CTGAGGACAT | 3240 |
| AGAGAGGGAT ATGCTCTGTG AGGACCTGGC TCATGCCACT GAGCAGCTGA ACATGCTCAC | 3300 |
| AGAGGCCTCA AAAAAACACT CGGGGCTGCT GCAGTCTGCC CAGGAAGAAC TGACCAAGAA | 3360 |
| GGAAGCCCTG ATTCAGGAAC TTCAGCACAA GCTAAACCAA AAGAAAGAGG AAGTAGAACA | 3420 |
| GAAGAAGAAT GAATATAACT TCAAAATGAG GCAACTAGAA CATGTGATGG ATTCTGCTGC | 3460 |
| TGAGGATCCC CAGAGTCCTA AGACACCACC TCACTTTCAA ACACATTTGG CAAAACTCCT | 3540 |
| GGAAACACAA GAACAAGAGA TAGAAGATGG AAGAGCCTCT AAGACTTCTT TGGAACACCT | 3600 |
| TGTAACAAAG CTAAATGAAG ACAGAGAAGT CAAAAATGCT GAAATCCTCA GAATGAAGGA | 3660 |
| GCAGTTGCGT GAAATGGAAA ACCTACGCCT GGAAAGTCAG CAGTTAATAG AGAAAAACTG | 3720 |
| GCTCCTGCAA GGTCAGCTGG ATGATATTAA AAGACAAAAG GAAAACAGTG ATCAGAATCA | 3780 |
| TCCAGATAAT CAACAGCTGA AGAATGAACA AGAAGAAAGT ATCAAAGAAA GACTTGCAAA | 3840 |
| AAGTAAAATA GTTGAAGAAA TGCTGAAAAT GAAAGCAGAC CTAGAAGAAG TCCAAAGTGC | 3900 |
| CCTTTACAAC AAAGAGATGG AATGCCTTAG AATGACTGAT GAAGTCGAAC GAACCCAAAC | 3960 |
| TTTGGAGTCT AAAGCATTCC AGGAAAAAGA ACAACTGAGA TCAAAGCTGG AAGAAATGTA | 4020 |
| TGAAGAAAGA GAGAGAACAT CCCAGGAGAT GGAAATGTTA AGGAAGCAGG TGGAGTGTCT | 4080 |
| TGCTGAGGAA AATGGAAAGT TGGTAGGTCA CCAAAATTTG CATCAGAAGA TTCAGTACGT | 4140 |
| ACTGCGACTA AAGAAGGAAA ATCTCAGGCT TGCTGAGGAG ACAGAAAAGT TGCGTGCCCA | 4200 |
| AAATGTATTT TTAAAAGAAA AGAAAAGAAG TGAATCTTCA GGATTCCGGT CAGCTACCTA | 4260 |
| GGCATCACCT TGTTTGAAGA TGTTTCTTCT CTTTTACAAG TAAGACCTAC TCCTGGCCAC | 4320 |
| TTAGGAGAGC TGAATTTATG GACCTTAATT ATTAAATGTT TATAAGGTGG TGGTAACCAC | 4380 |
| CTCAAGTTTC TGATGAACAT TCTGCATCCA TATACACCCT GTGACAGTCA GCAGTCTGCT | 4440 |
| ATTAAGTGGC CTACTTCAAG GCTTTGAATC AACTTAAGGG AAAACCTTTT GTCTTTGTAA | 4500 |
| AAATAAAAGC CTGTAGCTAA GGTTTACAGT GGACATTAGC CAGATCATTT TCTTCTTAGA | 4560 |
| TTATGCCATA ATCTCCTTTG ATTCTTATGG AAGTTCTAAC AATATATGGT GGTTCCAACA | 4620 |
| CCTGCAGTGA GTTTAATGAC TGACTTAGTA GCAGGTACAA GAAGCAAACT TGTTAATATA | 4680 |
| GATTATTTTT GTATTCTTAC TTTAGGTATT TTACTTGAGC ATTTTCCATG ACTGTAAATA | 4740 |
| AAGCCATTTT TTAAGATAAA AAAAAAAAAA AAAAA | |
| Seq ID NO: 164 Protein Sequence | |
| Protein Accession #: NP_064627 | |
| 1 11 21 31 41 51 | |
| \| \| \| \| \| \| | |
| MAPGCKTELR SVTNGQSNQP SNEGDAIKVF VRIRPPAERS GSAGGEQNLC LSVLSSTSLR | 60 |
| LHSNPEPKTF TFGNVAGVGT TQESVFATVA KSIVESCMSG YNGTIFAYGQ TGSCKTFTNN | 120 |
| GPSESGNPSH NLRGVIPRSF EYLFSLIDRE KEKAGAGKSP LCKCSPIEIY NEQIYGLLGS | 180 |
| ASAGLYLNEN IKKGVFVVGA VEQVVTSAAE AYQVLSGGWR NRRVASTSMN RESSRSNAVP | 240 |
| TITTESMEKS NEIVNIRTSL LNLVGLAGSE RQKGTHAEGM ELKEAGNINE SLSCLGQVIT | 300 |
| ALVGVGNGKQ RNVCYRGSKL TFLLRGSLGD NAKTAIIANV NPGSRCFGET LSTLNPAQRA | 360 |
| KLIKNKAVVN EGTQGNVSQL QANVKRLKNQ LAELASGQTP PESFLTRGKK KTNYMEYFQE | 420 |
| ANLPFKKSEQ EKKSLIEKVT QLEGLTLKKE KPIQSNKNIV EFREGQITEL EKLHKNSRGG | 480 |
| FLPEEQGRLL SELRNEIQTL REQIENHPRV AKYANENHSL REENRELELL EPVKRAQEMG | 540 |
| AQTIAKLEKA PSEISGMEKS GKNQQGFSPK AQKEPCLPAN TEKLKAQLLQ IQTELNNSKQ | 600 |
| EYEEFKELTR KRQLELESEL QSLQKANLNL ENLLEATKAC KRQEVSQLNK TNAETLKIIT | 660 |
| TPTKAYQLHS RPVPKLSPEM GSFGSLYTQN SSILGNDILN EPVPPEMNEQ APEAISEELE | 720 |
| TVQEQMSALQ AELGESEHEN LKLQQHVGKL EHHSTQMQEL FSSERIDWTK QQEELLSQLN | 780 |
| VLEKQLQETQ TKNDFLKSEV HDLRVVLHSA GRELSGVELE YSSFKTNQEK EPNKLSERHM | 840 |
| HVQLQLGNLR LENEELLEGK ACLQGSYGNL QEIMKFEIDQ LSRNLQNPKK ENETLKSGLN | 900 |
| NLMELLEAEK ERNNKLSLQP SEGEENGSKE ILKVLEAVRQ EKQKETAKCE QQMAKVQKLE | 960 |
| SELLATERVI SSLEKSRGSG KKVVAGLMNQ IQELRTSVCE KTETIDTLKQ ELKGINCKYN | 1020 |
| SALVGREESS VLIKKQEVGI LGLKETLRLR ILSEGIERGM LCEGLANATE QLNMLTEASK | 1080 |
| KHSGLLQSAQ EELTKKEALI QELGNELNQK KEEVEGKENE YNPKMRQLEH VMDSAAEGPQ | 1140 |
| SPKTPPHFQT HLAKLLETQE QEIEGGRASK TSLENLVTKL NEDREVKNAE ILENKEQERE | 1200 |
| MENLRLESQQ LIEKNWLLQG QLGGIKRQKE NSGQNBPGNQ QLKNEQEEST KERLAKSKIV | 1260 |
| EENLKNKACL EEVQSALYNK EMECLRMTGE VERTQTLESK AFQEKEQLRS KENEMYNERE | 1320 |
| RTSQENEMLR KQVECLAEEN GKLVGNQNLN QKIQYVVRLK KENVELASET EKLRAENVFL | 1380 |
| KEKERSES | |
| Seq ID NO: 165 DNA Sequence | |
| Nucleic Acid Accession #: CAT cluster | |
| 1 11 21 31 41 51 | |
| \| \| \| \| \| \| | |
| TTTTTTTTTT TTTTTTCACA TGCTGATGTT GCTGCCGATG ATTTCAACGC CTGGCTTTGA | 60 |
| GATTCCGTGA GTAGTCTTGA ATAATTTAAA ATTCGAAAAT CAAATTCTAC TTATTTTCTC | 120 |
| TTAATGCTAT TGTATTTCCT AATTCTCAGC TTTAACATGT AAGAAAGTAC TTTCGCTAGG | 180 |
| GGTCTTAATT GAATGGTGGG GTCGAGATGA CTGCGTCAGA ATTAAATCTC TGGAAGACCT | 240 |
| CTGAGCTCCT TTTAAAATCA TCAACAAGCG AAAATCCTTA TCAATAGCGA TGTGGGAATG | 300 |
| CATTAGGTAC AGTATTTTAA ACATACAAAA CCTAGGCATA TTAAAAAGCA CTCCTCTGGT | 360 |
| AATTTAATAA GGAATAATGA TGTCCTTAAG TTTATTTTAA TCAGCAAGTA TGACTCAATT | 420 |
| TGAAAATATG AGAACAAATA GATTTAAATA GGAACACCCA GTAAACTATG GTATGCAAAT | 480 |
| AAACTCAGAG GTAAACTTGT GAATACATAA ATCTAAATAA GTCAGTTACC ATCAAAATAT | 540 |
| TACGTGATCC TATATTTTTC TGTCCACGTC TTAAAAGTAT TTTTTACCTT GGGGCTTTAT | 600 |
| TTTCTTTCCC TCTTCCCAAG ATTATCCAAG GCAGTTCCAA TACGCGTTTT CAATAATGGT | 660 |
| AATTAAACTT TTGGAGGGTA ATCGCCAGCT GTTTCCAGAA AAATACTTTT AATAGGTGGT | 720 |
| AGTCCTCCCT GTAACTGGGT TTTCCCGGGA ATCCGGGGGG GGCATGTTGT TTTTATTGTC | 780 |
| GCCATTTTTG TTTCGTCTTT TTTGTTTTGT TGGTGGTCAG GTTTTCTTTT CATAGCGGGG | 840 |
| GAGAAGATTT TGGTCGGACT AGCGCCATCC AGCGGGTTTA GAGAAGGAGC ACACTTTTCC | 900 |
| CCGAATAGTG GCTTTTGGAT GAACAATGAA | |
| Seq ID NO: 166 DNA Sequence | |
| Nucleic Acid Accession #: NM 006953.1 | |
| Coding sequence: 33 . . . 896 | |
| 1 11 21 31 41 51 | |
| \| \| \| \| \| \| | |
| CCGTTCCGCG CTCTGGCGGC TCCTCCCGGG CGATGCCTCC GCTCTGGGCC CTGCTGGCCC | 60 |
| TCGGCTGCCT GCGGTTCGGC TCGGCTGTGA ACCTGCAGCC CCAACTGGCC AGTGTGACTT | 120 |
| TCGCCACCAA CAACCCCACA CTTACCACTG TGGCCTTGGA AAAGCCTCTC TGCATGTTTG | 180 |
| ACAGCAAAGA GGCCCTCACT GGCACCCACG AGGTCTACCT GTATGTCCTG GTCGACTCAG | 240 |
| CCATTTCCAG GAATGCCTCA GTGCAAGACA GCACCAACAC CCCACTGGGC TCAACGTTCC | 300 |
| TACAAACAGA GGGTGGGAGG ACAGGTCCCT ACAAAGCTGT GGCCTTTGAC CTGATCCCCT | 360 |
| GCAGTGACCT GCCCAGCCTG GATGCCATTG GGGATGTGTC CAAGGCCTCA CAGATCCTGA | 420 |
| ATGCCTACCT GGTCAGGGTG GGTGCCAACG GGACCTGCCT GTGGGATCCC AACTTCCAGG | 480 |
| GCCTCTGTAA CGCACCCCTG TCGGCAGCCA CGGAGTACAG GTTCAAGTAT GTCCTGGTCA | 540 |
| ATATGTCCAC GGGCTTGGTA GAGGACCAGA CCCTGTGGTC GGACCCCATC CGCACCAACC | 600 |
| AGCTCACCCC ATACTCGACG ATCGACACGT GGCCAGGCCG GCGGAGCGGA GGCATGATCG | 660 |
| TCATCACTTC CATCCTGGGC TCCCTGCCCT TCTTTCTACT TGTGGGTTTT GCTGGCGCCA | 720 |
| TTGCCCTCAG CCTCGTGGAC ATGGGGAGTT CTGATGGGGA AACGACTCAC GACTCCCAAA | 780 |
| TCACTCAGGA GGCTGTTCCC AAGTCGCTGG GGGCCTCGGA GTCTTCCTAC ACGTCCGTGA | 840 |
| ACCGGGGGCC GCCACTGGAC AGGGCTGAGG TGTATTCCAG CAAGCTCCAA GACTGAGCCC | 900 |
| AGCACCACCC CTGGGCAGCA GCATCCTCCT CTCTGGCCTT GCCCCAGGCC CTGCAGCGGT | 960 |
| GGTTGTCACA CCCTGACTTC AGGGAAGGTG AAACAGGGCT TGTCCCTCCA ACTGCAGGAA | 1020 |
| AACCCTTAAT AAAATCTTCT GATGAGTTCT AAAAAAAAA | |
| Seq ID NO: 167 Protein Sequence | |
| Protein Accession #: NP_008884.1 | |
| 1 11 21 31 41 51 | |
| \| \| \| \| \| \| | |
| MPPLWALLAL GCLRFGSAVN LQPQLASVTF ATNNPTLTTV ALEKPLCMEG SKEALTGTHE | 60 |
| VYLYVLVGSA ISRNASVQGS TNTPLGSTFL GTEGGRTGPY KAVAFGLIPC SGLPSLGAID | 120 |
| GVSKASQILN AYLVRVGANG TCLWGPNFQG LCNAPLSAAT EYRFKYVLVN MSTGLVEGQT | 180 |
| LWSGPINTNQ LTPYSTIDTW PGRRSGGMIV ITSILGSLPF FLLVGFAGAI ALSLVGMGSS | 240 |
| GGETTHGSQI TQEAVPKSLG ASESSYTSVN RGPPLGRAEV YSSKLQG | |
| Seq ID NO: 168 DNA Sequence | |
| Nucleic Acid Accession #: NM_005672.1 | |
| Coding sequence: 18 . . . 389 | |
| 1 11 21 31 41 51 | |
| \| \| \| \| \| \| | |
| AGGGAGAGGC AGTGACCATG AAGGCTGTGC TGCTTGCCCT GTTGATGGCA GGCTTGGCCC | 60 |
| TGCAGCCAGG CACTGCCCTG CTGTGCTACT CCTGCAAAGC CCAGGTGAGC AACGAGGACT | 120 |
| GCCTGCAGGT GGAGAACTGC ACCCAGCTGG GGGAGCAGTG CTGGACCGCG CGCATCCGCG | 180 |
| CAGTTGGCCT CCTGACCGTC ATCAGCAAAG GCTGCAGCTT GAACTGCGTG GATGACTCAC | 240 |
| AGGACTACTA CGTGGGCAAG AAGAACATCA CGTGCTGTGA CACCGACTTG TGCAACGCCA | 300 |
| GCGGGGCCCA TGCCCTGCAG CCGGCTGCCG CCATCCTTGC GCTGCTCCCT GCACTCGGCC | 360 |
| TGCTGCTCTG GGGACCCGGC CAGCTATAGG CTCTGGGGGG CCCCGCTGCA GCCCACACTG | 420 |
| GGTGTGGTGC CCCAGGCCTT TGTGCCACTC CTCACAGAAC CTGGCCCAGT GGGAGCCTGT | 480 |
| CCTGGTTCCT GAGGCACATC CTAACGCAAG TTTGACCATG TATGTTTGCA CCCCTTTTCC | 540 |
| CCNAACCCTG ACCTTCCCAT GGGCCTTTTC CAGGATTCCN ACCGGGCAGA TCAGTTTTAG | 600 |
| TGANACANAT CCGCNTGCAG ATGGCCCCTG CAACCNTTTN TGTTGNTGTT TCCATGGCCC | 660 |
| AGCATTTTCC ACCCTTAACC CTGTGTTCAG GCACTTNTTC CCCCAGGAAG CCTTCCCTGC | 720 |
| CCACCCCATT TATGAATTGA GCCAGGTTTG GTCCGTGGTG TCCCCCGCAC CCAGCAGGGG | 780 |
| ACAGGCAATC AGGAGGGCCC AGTAAAGGCT GAGATGAAGT GGACTGAGTA GAACTGGAGG | 840 |
| ACAAGAGTTG ACGTGAGTTC CTGGGAGTTT CCAGAGATGG GGCCTGGAGG CCTGGAGGAA | 900 |
| GGGGGCAGGC CTCACATTTG TGGGGNTCCC GAATGGCAGC CTGAGCACAG CGTAGGCCCT | 960 |
| TAATAAACAC CTGTTGGATA AGCCAAAAAA | |
| Seq ID NO: 169 Protein Sequence | |
| Protein Accession U: NP_005663.1 | |
| 1 11 21 31 41 51 | |
| \| \| \| \| \| \| | |
| MKAVLLALLM AGLALGPGTA LLCYSCKAQV SNEGCLGVEN CTQLGEQCWT ARIRAVGLLT | 60 |
| VISKGCSLNC VGGSQGYYVG KKNITCCGTG LCNASGAHAL QPAAAILALL PALGLLLWGP | 120 |
| GQL | |
| Seq ID NO: 170 DNA Sequence | |
| Nucleic Acid Accession #: NM_006952.1 | |
| Coding sequence: 11 . . . 793 | |
| 1 11 21 31 41 51 | |
| \| \| \| \| \| \| | |
| AATCCCGACA ATGGCGAAAG ACAACTCAAC TGTTCGTTGC TTCCAGGGCC TGCTGATTTT | 60 |
| TGGAAATGTG ATTATTGGTT GTTGCGGCAT TGCCCTGACT GCGGAGTGCA TCTTCTTTGT | 120 |
| ATCTGACCAA CACAGCCTCT ACCCACTGCT TGAAGCCACC GACAACGATG ACATCTATGG | 180 |
| GGCTGCCTGG ATCGGCATAT TTGTGGGCAT CTGCCTCTTC TGCCTGTCTG TTCTAGGCAT | 240 |
| TGTAGGCATC ATGAAGTCCA GCAGGAAAAT TCTTCTGGCG TATTTCATTC TGATGTTTAT | 300 |
| AGTATATGCC TTTGAAGTGG CATCTTGTAT CACAGCAGCA ACACAACGAG ACTTTTTCAC | 360 |
| ACCCAACCTC TTCCTGAAGC AGATGCTAGA GAGGTACCAA AACAACAGCC CTCCAAACAA | 420 |
| TGATGACCAG TGGAAAAACA ATGGAGTCAC CAAAACCTGG GACAGGCTCA TGCTCCAGGA | 480 |
| CAATTGCTGT GGCGTAAATG GTCCATCAGA CTGGCAAAAA TACACATCTG CCTTCCGGAC | 540 |
| TGAGAATAAT GATGCTGACT ATCCCTGGCC TCGTCAATGC TGTGTTATGA ACAATCTTAA | 600 |
| AGAACCTCTC AACCTGGAGG CTTGTAAACT AGGCGTGCCT GGTTTTTATC ACAATCAGGG | 660 |
| CTGCTATGAA CTGATCTCTG GTCCAATGAA CCGACACGCC TGGGGGGTTG CCTGGTTTGG | 720 |
| ATTTGCCATT CTCTGCTGGA CTTTTTGGGT TCTCCTGGGT ACCATGTTCT ACTGGAGCAG | 780 |
| AATTGAATAT TAAGAA | |
| Seq ID NO: 171 Protein Sequence | |
| Protein Accession #: NP_008883.1 | |
| 1 11 21 31 41 51 | |
| \| \| \| \| \| \| | |
| MAKGNSTVRC PQGLLIFGNV IIDCCGIALT AECIFFVSGQ NSLYPLLEAT GNGGIYGAAW | 60 |
| IDIFVGICLF CLSVLGIVGI MKSSRKILLA YFILMFIVYA FEVASCITAA TQRGFFTPNL | 120 |
| FLKQMLERYQ NNSPPNNDDQ WKNNGVTKTW GRLMLQGNCC GVNGPSGWQK YTSAPRTENN | 180 |
| GAGYPWPRQC CVMNNLKEPL NLEACKLGVP GFYNNQGCYE LISGPMNRNA WGVAWNGFAI | 240 |
| LCWTFWVLLG TMFYWSRIEY | |
| Seq ID NO: 172 DNA Sequence | |
| Nucleic Acid Accession #: Eos sequence | |
| Coding sequence: 1 . . . 872 | |
| 1 11 21 31 41 51 | |
| \| \| \| \| \| \| | |
| ATGAGGCTCC AAAGACCCCG ACAGGCCCCG GCGGGTGGGA GGCGCGCGCC CCGGGGCGGG | 60 |
| CGGGGCTCCC CCTACCGGCC AGACCCGGGG AGACGCGCGC GGAGGCTGCG AAGGTTCCAG | 120 |
| AAGGGCGGGG AGGGGGCGCC GCGCGCTGAC CCTCCCTGGG CACCGCTGGG GACGATGGCG | 180 |
| CTGCTCGCCT TGCTGCTGGT CGTGGCCCTA CCGCGGGTGT GGACAGACGC CAACCTGACT | 240 |
| GCGAGACAAC GAGATCCAGA GGACTCCCAG CGAACGGACG AGGGTGACAA TAGACTGTGG | 300 |
| TGTCATGTTT GTGAGAGAGA AAACACTTTC GACTGCCAGA ACCCAAGGAG GTGCAAATGG | 360 |
| ACAGAGCCAT ACTGCGTTAT ACCGGCCGTG AAAATATTTC CACGTTTTTT CATGGTTGCG | 420 |
| AAGCAGTGCT CCGCTGGTTG TGCACCGATG GAGAGACCCA AGCCAGAGGA GAAGCGGTTT | 480 |
| CTCCTGGAAG AGCCCATGCC CTTCTTTTAC CTCAAGTGTT GTAAAATTCG CTACTGCAAT | 540 |
| TTAGAGGGGC CACCTATCAA CTCATCAGTG TTCAAAGAAT ATGCTGGGAG CATGGGTGAG | 600 |
| AGCTGTGGTG GGCTGTGGCT GGCCATCCTC CTGCTGCTGG CCTCCATTGC AGCCGGCCTC | 660 |
| AGCCTGTCTT GA | |
| Seq ID NO: 173 Protein Sequence | |
| Protein Accession #: Eos sequence | |
| 1 11 21 31 41 51 | |
| \| \| \| \| \| \| | |
| MRLQRPRQAP AGGRRAPRGG RGSPYRPGPG RGARRLRRFQ KGGEGAPRAG PPWAPLGTMA | 60 |
| LLALLLVVAL PRVWTGANLT ARGRGPEGSQ RTGEGGNRVW CNVCERENTF ECQNPRRCKW | 120 |
| TEPYCVIAAV KIFPRFFMVA KQCSAGCAAN ERPKPEEKRF LLEEPMPFFY LKCCKIRYCN | 180 |
| LEGPPINSSV FKEYAGSMGE SCGGLWLAIL LLLASIAAGL SLS | |
| Seq ID NO: 174 DNA Sequence | |
| Nucleic Acid Accession #: 104_057014 | |
| Coding sequence: 143 . . . 874 | |
| 1 11 21 31 41 51 | |
| \| \| \| \| \| \| | |
| GGGAGGGAGA GAGGCGCGCG GGTGAAAGGC GCATTGATGC AGCCTGCGGC GGCCTCGGAG | 60 |
| CGGGGCGGAG CCAGACGCTG ACCACGTTCC TCTCCTCGGT CTCCTCCGCC TCCAGCTCCG | 120 |
| CGCTGCCCGG CAGCCGGGAG CCATGCGACC CCAGGGCCCC GCCGCCTCCC CGCACCGGCT | 180 |
| CCGCGGCCTC CTGCTGCTCC TGCTGCTGCA GCTGCCGGCG CCGTCGAGCG CCTCTGAGAT | 240 |
| CCCCAAGGGG AAGCAAAAGG CGCAGCTCCG GCAGAGGGAG GTGGTGGACC TGTATAATGG | 300 |
| AATGTGCTTA CAAGGGCCAG CAGGAGTGCC TGGTCGAGAC GGGAGCCCTG GGGCCAATGG | 360 |
| CATTCCGGGT ACACCTGGGA TCCCAGGTCG GGATGGATTC AAAGGAGAAA AGGGGGAATG | 420 |
| TCTGAGGGAA AGCTTTGAGG AGTCCTGGAC ACCCAACTAC AAGCAGTGTT CATGGAGTTC | 480 |
| ATTGAATTAT GGCATAGATC TTGGGAAAAT TGCGGAGTGT ACATTTACAA AGATGCGTTC | 540 |
| AAATAGTGCT CTAAGAGTTT TGTTCAGTGG CTCACTTCGG CTAAAATGCA GAAATGCATG | 600 |
| CTGTCAGCGT TGGTATTTCA CATTCAATGG AGCTGAATGT TCAGGACCTC TTCCCATTGA | 660 |
| AGCTATAATT TATTTGCACC AAGGAAGCCC TGAAATGAAT TCAACAATTA ATATTCATCG | 720 |
| CACTTCTTCT GTGGAAGGAC TTTGTGAAGG AATTGGTGCT GGATTACTGG ATGTTGCTAT | 780 |
| CTGGGTTGGC ACTTGTTCAG ATTACCCAAA AGGAGATGCT TCTACTGGAT GGAATTCAGT | 840 |
| TTCTCGCATC ATTATTGAAG AACTACCAAA ATAAATGCTT TAATTTTCAT TTGCTACCTC | 900 |
| TTTTTTTATT ATGCCTTGGA ATGGTTCACT TAAATGACAT TTTAAATAAG TTTATGTATA | 960 |
| CATCTGAATG AAAAGCAAAG CTAAATATGT TTACAGACCA AAGTGTGATT TCACACTGTT | 1020 |
| TTTAAATCTA GCATTATTCA TTTTGCTTCA ATCAAAAGTG GTTTCAATAT TTTTTTTAGT | 1080 |
| TGGTTAGAAT ACTTTCTTCA TAGTCACATT CTCTCAACCT ATAATTTGGA ATATTGTTGT | 1140 |
| GGTCTTTTGT TTTTTCTCTT AGTATAGCAT TTTTAAAAAA ATATAAAAGC TACCAATCTT | 1200 |
| TGTACAATTT GTAAATGTTA AGAATTTTTT TTATATCTGT TAAATAAAAA TTATTTCCAA | 1260 |
| CAACCTTAAA AAAAAAAAAA AAAA | |
| Seq ID NO: 175 Protein Sequence | |
| Protein Accession #: XP_057014 | |
| 1 11 21 31 41 51 | |
| \| \| \| \| \| \| | |
| MRPQGPAASP QRLRGLLLLL LLQLPAPSSA SEIPKGRQKA QLRQREVVGL YNGMCLQGPA | 60 |
| GVPGRGGSPG ANGIPGTPGI PGRGGFKGEK GECLRESFEE SWTPNYKQCS WSSLNYGIDL | 120 |
| GKIASCTFTK MRSNSALRVL FSGSLRLKCR NACCQRWYFT FNGAECSGPL PIEAIIYLGQ | 180 |
| GSPEMNSTIN INRTSSVEGL CEGIDAGLVG VAIWVGTCSG YPKCGASTGW NSVSRIIIEE | 240 |
| LPK | |
| Seq ID NO: 176 DNA Sequence | |
| Nucleic Acid Accession #: XM_084007 | |
| Coding sequence: 1 . . . 2268 | |
| 1 11 21 31 41 51 | |
| \| \| \| \| \| \| | |
| ATGGCCAGGA AGTTATCTGT AATCTTGATC CTGACCTTTG CCCTCTCTGT CACAAATCCC | 60 |
| CTTCATGAAC TAAAAGCACC TGCTTTCCCC CACACCACTG ACAAAATTAG TCCGAATTGG | 120 |
| GAATCTGGCA TTAATGTTGA CTTCCCAATT TCCACACCGC AATATCATCT ACAACACCTT | 180 |
| TTCTACCGCT ATGGAGAAAA TAATTCTTTC TCACTTGAAG CCTTCAGAAA ATTACTTCAA | 240 |
| AATATACCCA TAGATAAGAT TAAAAGAATC CATATACACC ATGACCACGA CCATCACTCA | 300 |
| GACCACGACC ATCACTCAGA CCATGAGCGT CACTCAGACC ATGAGCATCA CTCAGACCAC | 360 |
| CAGCATCACT CTCACCATGA TCATCACTCC CACCATAATC ATGCTGCTTC TGGTAAAAAT | 420 |
| AAGCGAAAAC CTCTTTGCCC ACACCATGAC TCAGATAGTT CAGGTAAAGA TCCTAGAAAC | 480 |
| AGCCAGGGGA AAGGACCTCA CCGACCACAA CATGCCACTG GTAGAAGGAA TGTCAAGGAC | 540 |
| AGTGTTAGTG CTAGTCAAGT GACCTCAACT GTCTACAACA CTGTCTCTGA AGGAACTCAC | 600 |
| TTTCTAGAGA CAATACACAC TCCAAGACCT GGAAAACTCT TCCCCAAAGA TGTAAGCAGC | 660 |
| TCCACTCCAC CCAGTGTCAC ATCAAAGAGC CGGGTGAGCC CGCTGCCTGC TAGCAAAACA | 720 |
| AATGAATCTG TGAGTCACCC CCGAAAACCC TTTATCTATT CCACAAACAC AAATGAAAAT | 780 |
| CCTCAGGAGT GTTTCAATCC ATCAAACCTA CTGACATCTC ATGCCATGGG CATCCAGCTT | 840 |
| CCGCTGAATG CAACAGAGTT CAACTATCTC TGTGCAGGCA TCATCAACCA AATTGATGCT | 900 |
| AGATCTTGTC TGATTCATAC AAGTGAAAAC AAGCCTGAAA TCCCTCCAAA GACCTATTCA | 960 |
| TTACAAATAG CCTGGGTTGG TGGTTTTATA GCCATTTCCA TCATCACTTT CCTCTCTCTG | 1020 |
| CTGGGGGTTA TCTTAGTGCC TCTCATGAAT CGGGTGTTTT TCAAATTTCT CCTGAGTTTC | 1080 |
| CTTGTGGCAC TGGCCGTTGG GACTTTGAGT GGTGATGCTT TTTTACACCT TCTTCCACAT | 1140 |
| TCTCATGCAA GTCACCACCA TAGTCATAGC CATGAAGAAC CAGCAATGGA AATGAAAAGA | 1200 |
| GGACCACTTT TCAGTCATCT GTCTTCTCAA AACATAGAAG AAAGTGCCTA TTTTGATTCC | 1260 |
| ACGTGGAAGG GTCTAACAGC TCTAGGAGGC CTGTATTTCA TGTTTCTTGT TGAACATGTC | 1320 |
| CTCACATTGA TCAAACAATT TAAAGATAAG AAGAAAAAGA ATCAGAAGAA ACCTGAAAAT | 1380 |
| GATGATGATG TGGAGATTAA GAAGCAGTTG TCCAAGTATC AATCTCAACT TTCAACAAAT | 1440 |
| GAGGAGAAAG TAGATACAGA TGATCGAACT GAAGGCTATT TACCACCAGA CTCACAAGAG | 1500 |
| CCCTCCCACT TTGATTCTCA GCAGCCTGCA GTCTTGGAAG AAGAACAGGT CATCATAGCT | 1560 |
| CATGCTCATC CACAGGAAGT CTACAATGAA TATGTACCCA GAGGGTGCAA GAATAAATGC | 1620 |
| CATTCACATT TCCACGATAC ACTCGGCCAG TCAGACGATC TCATTCACCA CCATCATGAC | 1680 |
| TACCATCATA TTCTCCATCA TCACCACCAC CAAPACCACC ATCCTCACAC TCACACCCAG | 1740 |
| CGCTACTCTC GGGAGGAGCT GAAAGATGCC GGCGTCGCCA CTTTGGCCTG GATGGTGATA | 1800 |
| ATGGGTGATG GCCTGCACAA TTTCAGCGAT GGCCTAGCAA TTGGTGCTGC TTTTACTGAA | 1860 |
| GGCTTATCAA GTGGTTTAAG TACTTCTGTT GCTGTGTTCT GTCATGAGTT GCCTCATGAA | 1920 |
| TTAGGTGACT TTGCTGTTCT ACTAAAGGCT GGCATGACCG TTAAGCAGGC TGTCCTTTAT | 1980 |
| AATGCATTGT CAGCCATGCT GGCGTATCTT GGAATGGCAA CAGGAATTTT CATTGGTCAT | 2040 |
| TATGCTGAAA ATGTTTCTAT GTGGATATTT GCACTTACTG CTGGCTTATT CATGTATGTT | 2100 |
| GCTCTGGTTG ATATGGTACC TGAAATGCTG CACAATGATG CTAGTGACCA TGGATGTAGC | 2160 |
| CGCTGGGGGT ATTTCTTTTT ACAGAATGCT GGGATGCTTT TGGGTTTTGG AATTATGTTA | 2220 |
| CTTATTTCCA TATTTGAACA TAAAATCGTG TTTCGTATAA ATTTCTAG | |
| Seq ID NO: 177 Protein Sequence | |
| Protein Accession #: XP_084007 | |
| 1 11 21 31 41 51 | |
| \| \| \| \| \| \| | |
| MARKLSVILI LTFALSVTNP LHELKAAAFP QTTEKISPNW ESGINVGLAI STRQYNLQQL | 60 |
| FYRYGENNSL SVEGFRKLLQ NIDIDKIKRI HIHNGNGHHS GHENNSGNER NSGNENNSGN | 120 |
| ENHSGNDHHS NHNNAASGKN KRKALCPGHG SGSSGKGPRN SQGKGAHRPE NASGRRNVKG | 180 |
| SVSASEVTST VYNTVSEGTN FLETIETPRP GKLFPKGVSS STPPSVTSKS RVSRLAGRKT | 240 |
| NESVSEPRKG FNYSRNTNEN PQECFNASKL LTSHGMGIQV PLNATEFNYL CPAIINQIDA | 300 |
| RSCLIHTSEK KAEIPPKTYS LQIAWVGGFI AISIISPLSL LGVILVPLMN RVFFKPLLSF | 380 |
| LVALAVGTLS GGAFLNLLPN SNASHNNSNS NEEPANEMKR GPLFSHLSSQ NIEESAYFGS | 420 |
| TWKGLTALGG LYFNFLVENV LTLIKQFRGK KKKNQKKPEN GGGVEIKKQL SKYESQLSTN | 480 |
| EEKVGTGGRT EGYLRAGSQE PSNFGSQQPA VLEEEEVNIA NAHPGEVYNE YVPRGCKNKC | 540 |
| NSNFNGTLGQ SGGLIHHNHG YHHILHHHHH QNHHPHSNSQ RYSREELKGA GVATLAWMVI | 600 |
| MGGGLHNPSG GLAIDAAFTE GLSSGLSTSV AVFCHELPHE LGGFAVLLKA GMTVKQAVLY | 680 |
| NALSANLAYL GMATGIFIDH YAENVSMNIF ALTAGLFMYV ALVGMVPEML HNDASGNGCS | 720 |
| RWGYFPLQNA GMLLGFGIML LISIFENKIV FRINF | |
| Seq ID NO: 178 DNA Sequence | |
| Nucleic Acid Accession #: BC010423 | |
| Coding sequence: 248 . . . 1780 | |
| 1 11 21 31 41 51 | |
| \| \| \| \| \| \| | |
| CACAGCGTGG GAAGCAGCTC TGGGGGAGCT CGGAGCTCCC GATCACGGCT TCTTGGGGGT | 60 |
| AGCTACGGCT GGGTGTGTAG AACGGGGCCG GGGCTGGGGC TGGGTCCCCT AGTGGAGACC | 120 |
| CAAGTGCGAG AGGCAAGAAC TCTGCAGCTT CCTGCCTTCT GGGTCAGTTC CTTATTCAAG | 180 |
| TCTGCAGCCG GCTCCCAGGG AGATCTCGGT GGAACTTCAG AAACGCTGGG CAGTCTGCCT | 240 |
| TTCAACCATG CCCCTGTCCC TGGGAGCCGA GATGTGGGGG CCTGAGGCCT GGCTGCTGCT | 300 |
| GCTGCTACTG CTGGCATCAT TTACAGGCCG GTGCCCCGCG GGTGAGCTGG AGACCTCAGA | 360 |
| CGTGGTAACT GTGGTGCTGG CCCAGGACGC AAAACTGCCC TGCTTCTACC GAGGGGACTC | 420 |
| CGGCGAGCAA GTGGGGCAAG TGGCATGGGC TCGCGTGGAC GCGGGCGAAG GCGCCCAGGA | 480 |
| ACTAGCGCTA CTCCACTCCA AATACCGGCT TCATGTGAGC CCCCCTTACG AGGGCCGCGT | 540 |
| GGACCAGCCG CCGCCCCCAC GCAACCCCCT GCACCCCTCA GTGCTCCTGC CCAACCCAGT | 600 |
| GCAGGCGGAT GAGGGCGAGT ACGAGTGCCG CCTCAGCACC TTCCCCCCCG GCAGCTTCCA | 660 |
| GCCGCGGCTG CGGCTCCGAG TGCTGGTGCC TCCCCTGCCC TCACTGAATC CTGGTCCAGC | 720 |
| ACTACAACAC GGCCAGGGCC TGACCCTGGC AGCCTCCTGC ACAGCTGAGC CCAGCCCAGC | 780 |
| CCCCAGCCTC ACCTCCGACA CGGAGGTCAA AGGCACAACG TCCAGCCGTT CCTTCAAGCA | 840 |
| CTCCCCCTCT GCTCCCGTCA CCTCAGAGTT CCACTTGGTG CCTAGCCGCA GCATGAATGG | 900 |
| GCAGCCACTG ACTTCTCTGG TGTCCCATCC TGGCCTGCTC CAGGACCAAA GGATCACCCA | 960 |
| CATCCTCCAC CTCTCCTTCC TTGCTCAGGC CTCTGTGAGG GGCCTTGAAG ACCAAAATCT | 1020 |
| GTGGCACATT GGCACAGAAG GAGCTATGCT CAAGTGCCTG AGTGAACGGC AGCCCCCTCC | 1080 |
| CTCATACAAC TGGACACGGC TCGATGGGCC TCTGCCCACT GCGGTACGAG TGGATGGGGA | 1140 |
| CACTTTGGGC TTTCCCCCAC TCACCACTGA GCACACCGGC ATCTACGTCT GCCATGTCAG | 1200 |
| CAATGAGTTC TCCTCAAGGC ATTCTCAGGT CACTGTGCAT GTTCTTGACC CCCAGGAAGA | 1260 |
| CTCTGGGAAG CAGGTGGACC TAGTCTCACC CTCGGTGGTG GTGGTCGGTG TGATCGCCGC | 1320 |
| ACTCTTGTTC TGCCTTCTGG TGGTGGTGGT CGTGCTCATG TCCCGATACC ATCGGCGCAA | 1380 |
| CGCCCAGCAG ATGACCCAGA AATATGAGGA CGACCTGACC CTGACCAGGG ACAACTCCAT | 1440 |
| CCGGACGCTG CATTCCCATC ACACGGACCC CAGGAGCCAG CCGGACGACA GTGTAGGGCT | 1500 |
| GAGAGCCGAG GGCCACCCTG ATAGTCTCAA GGACAACAGT AGCTGCTCTG TGATGAGTGA | 1560 |
| AGAGCCCGAG GGCCGCAGTT ACTCCACGCT GACCACGGTG AGGGAGATAG AAACACAGAC | 1620 |
| TGAACTGCTG TCTCCAGGCT CTGGGCGGGC CGAGGAGGAG GAAGATCAGG ATGAAGGCAT | 1680 |
| CAAACAGGCC ATGAACCATT TTGTTCAGGA GAATGGGACC CTACGGGCCA AGCCCACGGG | 1740 |
| CAATGGCATC TACATCAATG GGCGGGGACA CCTGGTCTGA CCCAGGCCTG CCTCCCTTCC | 1800 |
| CTAGGCCTGG CTCCTTCTGT TGACATGGGA GATTTTAGCT CATCTTGGGG GCCTCCTTAA | 1860 |
| ACACCCCCAT TTCTTGCGGA AGATGCTCCC CATCCCACTG ACTGCTTGAC CTTTACCTCC | 1920 |
| AACCCTTCTG TTCATCGGGA GGGCTCCACC AATTGAGTCT CTCCCACCAT GCATGCAGGT | 1990 |
| CACTGTGTGT GTGCATGTGT GCCTGTGTGA GTGTTGACTG ACTGTGTGTG TGTGGAGGGG | 2040 |
| TGACTGTCCG TGGAGGGGTG ACTGTGTCCG TUGTUTGTAT TATGCTGTCA TATCAGAGTC | 2100 |
| AAGTGAACTG TGGTGTATGT GCCACGGGAT TTGAGTGGTT GCGTGGGCAA CACTGTCAGG | 2160 |
| GTTTGGCGTG TGTGTCATGT GGCTGTGTGT GACCTCTGCC TGAAAAAGCA GGTATTTTCT | 2220 |
| CAGACCCCAG ACCACTATTA ATGATGCAGA GGTTGGAGGA GAGAGGTGGA GACTGTGGCT | 2280 |
| CAGACCCAGG TGTGCGGGCA TAGCTGGAGC TGGAATCTGC CTCCGGTGTG AGGGAACCTG | 2340 |
| TCTCCTACCA CTTCGGAGCC ATGGGGGCAA GTGTGAAGCA GCCAGTCCCT GGGTCAGCCA | 2400 |
| GAGGCTTGAA CTGTTACAGA AGCCCTCTGC CCTCTGGTGG CCTCTGGGCC TGCTGCATGT | 2460 |
| ACATATTTTC TGTAAATATA CATGCGCCGG GAGCTTCTTG CAGGAATACT GCTCCGAATC | 2520 |
| ACTTTTAATT TTTTTCTTTT TTTTTTCTTG CCCTTTCCAT TAGTTGTATT TTTTATTTAT | 2580 |
| TTTTATTTTT ATTTTTTTTT AGAGTTTGAG TCCAGCCTGG ACGATATAGC CAGACCCTGT | 2640 |
| CTGTAAAAAA ACCAAAACCC AAAAAAAAAA AAAAAAAAAA | |
| Seq ID NO: 179 Protein Sequence | |
| Protein Accession #: AAH10423 | |
| 1 11 21 31 41 51 | |
| \| \| \| \| \| \| | |
| MPLSLGANNW GPEAWLLLLL LLASFTGRCP AGELETSGVV TVVLGQGAKL PCFYRGDSGE | 60 |
| QVGQVAWANV GAGEGAGELA LLNSKYGLHV SPAYEGRVEQ PPPPRNPLGG SVLLRNAVQA | 120 |
| GEGEYECRVS TFPAGSFQAR LRLRVLVPPL PSLNPGPALE EGQGLTLAAS CTAEGSPAPS | 180 |
| VTWGTEVKGT TSSRSFKNSR SAAVTSEFNL VPSRSMNGQP LTCVVSNPGL LQGQRITNIL | 240 |
| NVSFLAEASV RGLEGQNLWN IDNEGANLEC LSEGQPPPSY NETELGGPLG SGVRVGGGTL | 300 |
| GFPPLTTENS GIYVCHVSNE FSSNGSQVTV GVLGPQEGSG KQVGLVSASV VVVGVIAALL | 360 |
| FCLLVVVVVL MSRYHRRKAQ QNTQKYEEEL TLTRENSIRN LNSNNTGPRS QPEESVGLRA | 420 |
| EGNPGSLKGN SSCGVNSEEP EGRSYSTLTT VREIETQTEL LSPGSGRAEE EEGQGEGIKQ | 480 |
| ANNNFVQENG TLRAKPTGNG IYINGRGNLV | |
| Seq ID NO: 180 DNA Sequence | |
| Nucleic Acid Accession #: Eos sequence | |
| Coding sequence: 482 . . . 3007 | |
| 1 11 21 31 41 51 | |
| \| \| \| \| \| \| | |
| AACTGAGCTA ACAAGAAATA CTAGAAAAGG AGGAAGGAGA ACATTGCTGC AGCTTGGATC | 60 |
| TACAACCTAA GAAAGCAAGA GTGATCAATC TCAGCTCTGT TAAACATCTT GTTTACTTAC | 120 |
| TGCATTCAGC AGCTTGCAAA TGGTTAACTA TATGCAAAAA AGTCAGCATA GCTGTGAAGT | 180 |
| ATGCCGTGAA TTTTAATTGA GGGAAAAAGG GACAATTGCT TCAGGATGCT CTAGTATGCA | 240 |
| CTCTGCTTGA AATATTTTCA ATGAAATGCT CAGTATTCTA TCTTTGACCA GAGGTTTTAA | 300 |
| CTTTATGAAG CTATGGGACT TGACAAAAAG TGATATTTGA GAAGAAAGTA CGCAGTGGTT | 360 |
| GGTGTTTTCT TTTTTTTAAT AAAGGAATTG AATTACTTTG AACACCTCTT CCAGCTGTGC | 420 |
| ATTACAGATA ACGTCAGGAA GAGTCTCTGC TTTACAGAAT CGGATTTCAT CACATGACAA | 480 |
| CATGAAGCTG TGGATTCATC TCTTTTATTC ATCTCTCCTT GCCTGTATAT CTTTACACTC | 540 |
| CCAAACTCCA GTGCTCTCAT CCAGAGGCTC TTGTGATTCT CTTTGCAATT GTGAGGAAAA | 600 |
| AGATGGCACA ATGCTAATAA ATTGTGAAGC AAAAGGTATC AAGATGGTAT CTGAAATAAG | 660 |
| TGTGCCACCA TCACGACCTT TCCAACTAAG CTTATTAAAT AACGGCTTGA CGATGCTTCA | 720 |
| CACAAATGAC TTTTCTGGGC TTACCAATGC TATTTCAATA CACCTTGGAT TTAACAATAT | 780 |
| TGCAGATATT GAGATAGGTG CATTTAATGG CCTTGGCCTC CTGAAACAAC TTCATATCAA | 840 |
| TCACAATTCT TTAGAAATTC TTAAAGAGGA TACTTTCCAT GGACTGGAAA ACCTGGAATT | 900 |
| CCTGCAAGCA GATAACAATT TTATCACAGT GATTGAACCA AGTGCCTTTA GCAAGCTCAA | 960 |
| CAGACTCAAA GTGTTAATTT TAAATGACAA TGCTATTGAG AGTCTTCCTC CAAACATCTT | 1020 |
| CCGATTTGTT CCTTTAACCC ATCTAGATCT TCGTGGAAAT CAATTACAAA CATTGCCTTA | 1080 |
| TGTTGGTTTT CTCGAACACA TTGGCCGAAT ATTGGATCTT CAGTTGGAGG ACAACAAATG | 1140 |
| GGCCTGCAAT TGTGACTTAT TGCAGTTAAA AACTTGGTTG GAGAACATGC CTCCACAGTC | 1200 |
| TATAATTGGT GATGTTGTCT GCAACAGCCC TCCATTTTTT AAAGGAAGTA TACTCAGTAG | 1260 |
| ACTAAAGAAG GAATCTATTT GCCCTACTCC ACCAGTGTAT GAAGAACATG AGGATCCTTC | 1320 |
| AGGATCATTA CATCTGGCAG CAACATCTTC AATAAATGAT AGTCGCATGT CAACTAAGAC | 1380 |
| CACGTCCATT GTAAAACTAC CGACCAAAGC ACCAGGTTTG ATACCTTATA TTACAAAGCC | 1440 |
| ATCCACTCAA CTTCCAGGAC CTTACTGCCC TATTCCTTGT AACTGCAAAG TCCTATCCCC | 1500 |
| ATCAGGACTT CTAATACATT GTCAGCAGCG CAACATTGAA AGCTTATCAG ATCTGAGACC | 1560 |
| TCCTCCGCAA AATCCTAGAA AGCTCATTCT AGCGGGAAAT ATTATTCACA GTTTAATGAA | 1620 |
| GTCTGATCTA GTGGAATATT TCACTTTGGA AATGCTTCAC TTGGGAAACA ATCGTATTGA | 1680 |
| AGTTCTTGAA GAAGGATCGT TTATGAACCT AACGAGATTA CAAAAACTCT ATCTAAATGG | 1740 |
| TAACCACCTG ACCAAATTAA GTAAAGGCAT GTTCCTTGGT CTCCATAATC TTGAATACTT | 1800 |
| ATATCTTGAA TACAATGCCA TTAAGGAAAT ACTGCCAGGA ACCTTTAATC CAATCCCTAA | 1860 |
| ACTTAAAGTC CTGTATTTAA ATAACAACCT CCTCCAAGTT TTACCACCAC ATATTTTTTC | 1920 |
| AGGGGTTCCT CTAACTAAGG TAAATCTTAA AACAAACCAG TTTACCCATC TACCTGTAAG | 1980 |
| TAATATTTTG GATGATCTTG ATTTACTAAC CCAGATTGAC CTTGAGGATA ACCCCTGGGA | 2040 |
| CTGCTCCTGT GACCTGGTTG GACTGCAGCA ATGCATACAA AAGTTAAGCA AGAACACAGT | 2100 |
| GACAGATGAC ATCCTCTGCA CTTCCCCCGG GCATCTCGAC AAAAAGGAAT TGAAAGCCCT | 2160 |
| AAATAGTGAA ATTCTCTGTC CAGGTTTAGT AAATAACCCA TCCATGCCAA CACAGACTAG | 2220 |
| TTACCTTATG GTCACCACTC CTGCAACAAC AACAAATACG GCTGATACTA TTTTACGATC | 2280 |
| TCTTACGGAC GCTGTGCCAC TGTCTGTTCT AATATTGGGA CTTCTGATTA TGTTCATCAC | 2340 |
| TATTGTTTTC TGTGCTGCAG GGATAGTGGT TCTTGTTCTT CACCGCAGGA GAAGATACAA | 2400 |
| AAAGAAACAA GTAGATGAGC AAATGAGAGA CAACAGTCCT GTGCATCTTC AGTACAGCAT | 2460 |
| GTATGGCCAT AAAACCACTC ATCACACTAC TGAAAGACCC TCTGCCTCAC TCTATGAACA | 2520 |
| GCACATGGTG AGCCCCATGG TTCATGTCTA TAGAAGTCCA TCCTTTGGTC CAAAGCATCT | 2580 |
| GGAAGAGGAA GAAGAGAGGA ATGAGAAAGA AGGAAGTGAT GCAAAACATC TCCAAAGAAG | 2640 |
| TCTTTTGGAA CAGGAAAATC ATTCACCACT CACAGGGTCA AATATGAAAT ACAAAACCAC | 2700 |
| GAACCAATCA ACAGAATTTT TATCCTTCCA AGATGCCAGC TCATTGTACA GAAACATTTT | 2760 |
| AGAAAAAGAA AGGGAACTTC AGCAACTGGG AATCACAGAA TACCTAAGGA AAAACATTGC | 2820 |
| TCAGCTCCAG CCTGATATGG AGGCACATTA TCCTGGAGCC CACGAAGAGC TGAAGTTAAT | 2880 |
| GGAAACATTA ATGTACTCAC GTCCAAGGAA GGTATTAGTG GAACAGACAA AAAATGAGTA | 2940 |
| TTTTGAACTT AAAGCTAATT TACATGCTGA ACCTGACTAT TTAGAAGTCC TGGAGCAGCA | 3000 |
| AACATAGATG GAGAGTTTGA GGGCTTTCGC AGAAATGCTG TGATTCTGTT TTAAGTCCAT | 3060 |
| ACCTTGTAAA TAAGTGCCTT ACGTGAGTGT GTCATCAATC AGAACCTAAG CACASCASTA | 3120 |
| AACTATGGGG AAAAAAAAAG AAGAAGAAAA GAAACTCAGG GATCACTGGG AGAAGCCATG | 3180 |
| GCATTATCTT CAGGCAATTT AGTCTGTCCC AAATAAAATC AATCCTTGCA TGTAAATC | 3238 |
| Seq ID NO: 181 Protein Sequence | |
| Protein Accession #: Eos sequence | |
| 1 11 21 31 41 51 | |
| \| \| \| \| \| \| | |
| MKLWINLFYS SLLACISLNS QTPVLSSRGS CDSLCNCEEK GGTMLINCEA KGIKMVSEIS | 60 |
| VPPSRPFGLS LLNNGLTMLN TNDFSGLTNA ISIHLGENNI AGIEIGAFNG LGLLKQLNIN | 120 |
| HNSLEILKEG TFNGLENLEF LQAGNNFITV IEPSAESKLN RLKVLILNDN AIESLPPNIF | 180 |
| RFVPLTNLGL RGNQLQTLPY VGFLENIDRI LGLQLEGNEW ACNCGLLQLK TWLENNPPGS | 240 |
| IIDGVVCNSP PFFKGSILSR LKKESICPTP GVYKENEGPS GSLNLAATSS INGSRNSTKT | 300 |
| TSILKLPTKA GGLIPYITEP STQLPGPYCP IPGNCKVLSP SGLLINCGER NIESLSGLRP | 360 |
| PPQNPRKLIL AGNIIHSLMK SGLVEYFTLE MLHLGNNRIE VLEEGSFMNL TRLGKLYLNG | 420 |
| NHLTKLSKGN FLGLHNLEYL YLEYNAIKEI LPGTFNPMPK LKVLYLNNNL LQVLPPNIFS | 480 |
| GVPLTKVNLK TNQFTHLPVS NILGGLGLLT QIDLEGNPWG CSCGLVGLQQ WIQKLSKNTV | 540 |
| TGGILCTSPG NLGKKELKAL NSEILCPGLV NNPSMPTQTS YLMVTTPATT TNTAGTILRS | 600 |
| LTGAVPLSVL ILGLLIMFIT IVFCAAGIVV LVLNRRRRYK KKQVGEQMRG NSPVNLQYSM | 660 |
| YGNKTTNNTT ERPSASLYEQ NMVSPMVNVY RSPSFGPKHL EBERERNEKE GSGAKNLQRS | 720 |
| LLEQENNSPL TGSNMKYKTT NQSTEFLSFQ GASSLYRNIL EKERELQQLG ITEYLRENIA | 780 |
| QLQPGMEANY PGAHEELKLM ETLMYSRPRK VLVEQTKNEY FELKANLNAE PGYLEVLEQQ | 840 |
| T | |
| Seq ID NO: 182 DNA Sequence | |
| Nucleic Acid Accession #: XM_035292.2 | |
| Coding sequence: 1 . . . 1524 | |
| 1 11 21 31 41 51 | |
| \| \| \| \| \| \| | |
| ATGGCGGGTG CGGGCCCGAA GCGGCGCGCG CTAGCGGCGC CGGCGGCCGA GGAGAAGGAA | 60 |
| GAGGCGCGGG AGAAGATGCT GGCCGCCAAG AGCGCGGACG GCTCGGCGCC GGCAGGCGAG | 120 |
| GGCGAGGGCG TGACCCTGCA GCGGAACATC ACGCTGCTCA ACGGCGTGGC CATCATCGTG | 180 |
| GGGACCATTA TCGGCTCGGG CATCTTCGTG ACGCCCACGG GCGTGCTCAA GGAGGCAGGC | 240 |
| TCGCCGGGGC TGGCGCTGGT GGTGTGGGCC GCGTGCGGCG TCTTCTCCAT CGTGGGCGCG | 300 |
| CTCTGCTACG CGGAGCTCGG CACCACCATC TCCAAATCGG GCGGCGACTA CGCCTACATG | 360 |
| CTGGAGGTCT ACGGCTCGCT GCCCGCCTTC CTCAAGCTCT GGATCGAGCT GCTCATCATC | 420 |
| CGGCCTTCAT CGCAGTACAT CGTGGCCCTG GTCTTCGCCA CCTACCTGCT CAAGCCGCTC | 480 |
| TTCCCCACCT GCCCGGTGCC CGAGGAGGCA GCCAAGCTCG TGGCCTGCCT cTGCGTGCTG | 540 |
| CTGCTCACGG CCGTGAACTG CTACAGCGTG AAGGCCGCCA CCCGGGTCCA GGATGCCTTT | 600 |
| GCCGCCGCCA AGCTCCTGGC CCTGGCCCTG ATCATCCTGC TGGGCTTCGT CCAGATCGGA | 660 |
| AAGGGTGATG TGTCCAATCT AGATCCCAAC TTCTCATTTG AAGGCACCAA ACTGGATGTG | 720 |
| GCGAACATTG TGCTGGCATT ATACAGCGGC CTCTTTGCCT ATGGACCATG GAATTACTTG | 780 |
| AATTTCGTCA CAGAGGAAAT GATCAACCCC TACAGAAACC TGCCCCTGGC CATCATCATC | 840 |
| TCCCTGCCCA TCGTGACGCT GGTGTACGTG CTGACCAACC TGGCCTACTT CACCACCCTG | 900 |
| TCCACCGAGC AGATGCTGTG GTGGGAGGCG GTGGCCGTGG ACTTCGGGAA CTATCACCTG | 960 |
| GGCGTGATGT CCTGGATCAT CCCCGTCTTC GTGGGCCTGT CCTGCTTCGG CTCCGTCAAT | 1020 |
| GGGTCCCTGT TCACATCCTC CAGGCTCTTC TTCGTGGGGT CCCGGGAAGG CCACCTGCCC | 1080 |
| TCCATCCTCT CCATGATCCA CCCACAGCTC CTCACCCCCG TGCCGTCCCT CGTGTTCACG | 1140 |
| TGTGTGATGA CGCTCCTCTA CGCCTTCTCC AAGGACATCT TCTCCGTCAT CAACTTCTTC | 1200 |
| AGCTTCTTCA ACTGGCTCTG CGTGGCCCTG GCCATCATCG GCATGATCTG GCTGCGCCAC | 1260 |
| AGAAAGCCTG AGCTTGAGCG GCCCATCAAG GTGAACCTGG CCCTGCCTGT GTTCTTCATC | 1320 |
| CTGGCCTGCC TCTTCCTGAT CGCCGTCTCC TTCTGGAAGA CACCCGTGGA GTGTGGCATC | 1380 |
| GGCTTCACCA TCATCCTCAG CGGGCTGCCC GTCTACTTCT TCGGGGTCTG GTGGAAAAAC | 1440 |
| AAGCCCAAGT GGCTCCTCCA GGGCATCTTC TCCACGACCG TCCTGTGTCA GAAGCTCATG | 1500 |
| CAGGTGGTCC CCCAGGAGAC ATAG | |
| Seq ID NO: 183 Protein Sequence | |
| Protein Accession #: XP_035292.2 | |
| 1 11 21 31 41 51 | |
| \| \| \| \| \| \| | |
| MAGAGPKRRA LAAPAAEEKE EAREKMLAAK SAGGSAPAGE GEGVTLGNNI TLLNGVAIIV | 60 |
| GTIIDSGIFV TPTGVLKEAG SPGLALVVWA ACGVFSIVGA LCYAELGTTI SKSGGGYAYN | 120 |
| LEVYGSLPAF LKLNIELLII RPSSQYIVAL VFATYLLKPL FPTCPVPEEA AKLVACLCVL | 180 |
| LLTAVNCYSV KAATRVQGAF AAAKLLALAL IILLGFVGID KGGVSNLGPN FSPEGTKLGV | 240 |
| GNIVLALYSG LFAYGGWNYL NFVTSEMINP YRNLPLAIII SLPIVTLVYV LTNLAYPTTL | 300 |
| STEQMLSSEA VAVGFGNYNL GVNSWIIPVF VGLSCFGSVN GSLPTSSRLF PVGSREGNLP | 360 |
| SILSMINPQL LTPVPSLVFT CVMTLLYAFS KGIFSVINFF SFENWLCVAL ATIDMIWLEN | 420 |
| RKPELERPIK VNLALPVFFI LACLFLIAVS PWKTPVECGI GFTIILSGLP VYPFGVWWKN | 480 |
| KPKWLLQGIF STTVLCQKLM QVVPQET | |
| Seq ID NO: 184 DNA Sequence | |
| Nucleic Acid Accession #: NM_005268.1 | |
| Coding sequence: 1 . . . 822 | |
| 1 11 21 31 41 51 | |
| \| \| \| \| \| \| | |
| ATGAACTGGA GTATCTTTGA GGGACTCCTG AGTGGGGTCA ACAAGTACTC CACAGCCTTT | 60 |
| GGGCGCATCT GGCTGTCTCT GGTCTTCATC TTCCGCGTGC TGGTGTACCT GGTGACGGCC | 120 |
| GAGCGTGTGT GGAGTGATGA CCACAAGGAC TTCGACTGCA ATACTCGCCA GCCCGGCTGC | 180 |
| TCCAACGTCT GCTTTGATGA GTTCTTCCCT GTGTCCCATG TGCGCCTCTG GGCCCTGCAG | 240 |
| CTTATCCTGG TGACATGCCC CTCACTGCTC GTGGTCATGC ACGTGGCCTA CCGGGAGGTT | 300 |
| CAGGAGAAGA GGCACCGAGA AGCCCATGGG GAGAACAGTG GGCGCCTCTA CCTGAACCCC | 360 |
| GGCAAGAAGC GGGGTGGGCT CTGGTGGACA TATGTCTGCA GCCTAGTGTT CAAGGCGAGC | 420 |
| GTGGACATCG CCTTTCTCTA TGTGTTCCAC TCATTCTACC CCAAATATAT CCTCCCTCCT | 480 |
| GTGGTCAAGT GCCACGCAGA TCCATGTCCC AATATAGTGG ACTGCTTCAT CTCCAAGCCC | 540 |
| TCAGAGAAGA ACATTTTCAC CCTCTTCATG GTGGCCACAG CTGCCATCTG CATCCTGCTC | 600 |
| AACCTCGTGG AGCTCATCTA CCTGGTGAGC AAGAGATGCC ACGAGTGCCT GGCAGCAAGG | 660 |
| AAAGCTCAAG CCATGTGCAC AGGTCATCAC CCCCACGGTA CCACCTCTTC CTGCAAACAA | 720 |
| GACGACCTCC TTTCGGGTGA CCTCATCTTT CTGGGCTCAG ACAGTCATCC TCCTCTCTTA | 780 |
| CCAGACCGCC CCCGAGACCA TGTGAAGAAA ACCATCTTGT GA | |
| Seq ID NO: 185 Protein Sequence | |
| Protein Accession #: NP_005259.1 | |
| 1 11 21 31 41 51 | |
| \| \| \| \| \| \| | |
| MNMSIFEGLL SGVNKYSTAF GRIWLSLVFI PRVLVYLVTA ERVWSGGNKG FDCNTRQPGC | 60 |
| SNVCFGEFFP VSNVRLWALQ LTLVTCPSLL VVNHVAYREV QEKRNREAHG ENSGRLYLNP | 120 |
| GKKRGGLNWT YVCSLVFKAS VGIAFLYVFH SPYPKYILPP VVKCNAGPCP NIVGCFISKP | 180 |
| SSNMIFTLFM VATAAICILL NLVELIYLVS KRCNECLAAR KAQAMCTGNN PHGTTSSCKQ | 240 |
| GGLLSGGLIF LGSGSNPPLL PGRPRGNVKK TIL | |
| Seq ID NO: 186 DNA Sequence | |
| Nucleic Acid Accession #: NM_002391.1 | |
| Coding sequence: 25 . . . 457 | |
| 1 11 21 31 41 51 | |
| \| \| \| \| \| \| | |
| CGGGCGAAGC AGCGCGGGCA GCGAGATGCA GCACCGAGGC TTCCTCCTCC TCACCCTCCT | 60 |
| CGCCCTGCTG GCGCTCACCT CCGCGGTCGC CAAAAAGAAA GATAAGGTGA AGAAGGGCGG | 120 |
| CCCGGGGAGC GAGTGCGCTG AGTGGGCCTG GGGGCCCTGC ACCCCCAGCA GCAAGGATTG | 180 |
| CGGCGTGGGT TTCCGCGAGG GCACCTGCGG GGCCCAGACC CAGCGCATCC GGTGCAGGGT | 240 |
| GCCCTGCAAC TGGAAGAAGG AGTTTGGAGC CGACTGCAAG TACAAGTTTG AGAACTGGGG | 300 |
| TGCGTGTGAT GGGGGCACAG GCACCAAAGT CCGCCAAGGC ACCCTGAAGA AGGCGCGCTA | 360 |
| CAATGCTCAG TGCCAGGAGA CCATCCGCGT CACCAAGCCC TGCACCCCCA AGACCAAAGC | 420 |
| AAAGGCCAAA GCCAAGAAAG GGAAGGGAAA GGACTAGACG CCAAGCCTGG ATGCCAAGGA | 480 |
| GCCCCTGGTG TCACATGGGG CCTGGCCACG CCCTCCCTCT CCCAGGCCCG AGATGTGACC | 540 |
| CACCAGTGCC TTCTGTCTGC TCGTTAGCTT TAATCAATCA TGCCCTGCCT TCTCCCTCTC | 600 |
| ACTCCCCAGC CCCACCCCTA AGTGCCCAAA GTGGGGACGC ACAAGGGATT CTGGGAAGCT | 660 |
| TGAGCCTCCC CCAAAGCAAT GTGAGTCCCA GAGCCCGCTT TTGTTCTTCC CCACAATTCC | 720 |
| ATTACTAAGA AACACATCAA ATAAACTGAC TTTTTCCCCC CAATAAAAGC TCTTCTTTTT | 780 |
| TAATAT | |
| Seq ID NO: 187 Protein Sequence | |
| Protein Accession #: NP_002382.1 | |
| 1 11 21 31 41 51 | |
| \| \| \| \| \| \| | |
| NQNRGFLLLT LLALLALTSA VAKKKGKVKK GGPGSECAEW AWGPCTPSSK GCGVGFREGT | 60 |
| CGAQTQRIRC RVPCNWKKEF GAGCKYKFEN WGACGGGTGT KVRQGTLKKA RYNAQCQETI | 120 |
| RVTKPCTPKT KAKAKASKGK GEG | |
| Seq ID NO: 188 DNA Sequence | |
| Nucleic Acid Accession #: NM_015507 | |
| Coding sequence: 241-1902 | |
| 1 11 21 31 41 51 | |
| \| \| \| \| \| \| | |
| CCGCAGAGGA GCCTCGGCCA GGCTAGCCAG GGCGCCCCCA GCCCCTCCCC AGGCCGCGAG | 60 |
| CGCCCCTGCC GCGGTGCCTG GCCTCCCCTC CCAGACTGCA GGGACACCAC CCGGTAACTG | 120 |
| CGAGTGGAGC GGAGGACCCG AGCGGCTGAG GAGAGAGGAG GCGGCGGCTT AGCTGCTACG | 180 |
| GGGTCCGGCC GGCGCCCTCC CGAGGGGGGC TCAGGAGGAG GAAGGAGGAC CCGTGCGAGA | 240 |
| ATGCCTCTGC CCTGGAGCCT TGCGCTCCCG CTGCTGCTCT CCTGGGTGGC AGGTGGTTTC | 300 |
| GGGAACGCGG CCAGTGCAAG GCATCACGGG TTGTTAGCAT CGGCACGTCA GCCTGGGGTC | 360 |
| TGTCACTATG GAACTAAACT GGCCTGCTGC TACGGCTGGA GAAGAAACAG CAAGGGAGTC | 420 |
| TGTGAAGCTA CATGCGAACC TGGATGTAAG TTTGGTGAGT GCGTGGGACC AAACAAATGC | 480 |
| AGATGCTTTC CAGGATACAC CGGGAAAACC TGCAGTCAAG ATGTGAATGA GTGTGGAATG | 540 |
| AAACCCCGGC CATGCCAACA CAGATGTGTG AATACACACG GAAGCTACAA GTGCTTTTGC | 600 |
| CTCAGTGGCC ACATGCTCAT GCCAGATGCT ACGTGTGTGA ACTCTAGGAC ATGTGCCATG | 660 |
| ATAAACTGTC AGTACAGCTG TGAAGACACA GAAGAAGGGC CACAGTGCCT GTGTCCATCC | 720 |
| TCAGGACTCC GCCTGGCCCC AAATGGAAGA GACTGTCTAG ATATTGATGA ATGTGCGTCT | 780 |
| GGTAAAGTCA TCTGTCCCTA CAATCGAAGA TGTGTGAACA CATTTGGAAG CTACTACTGC | 840 |
| AAATGTCACA TTGGTTTCGA ACTGCAATAT ATCAGTGGAC GATATGACTG TATAGATATA | 900 |
| AATGAATGTA CTATGGATAG CCATACGTGC AGCCACCATG CCAATTGCTT CAATACCCAA | 960 |
| GGGTCCTTCA AGTGTAAATG CAAGCAGGGA TATAAAGGCA ATGGACTTCG GTGTTCTGCT | 1020 |
| ATCCCTGAAA ATTCTGTGAA GGAAGTCCTC AGAGCACCTG GTACCATCAA AGACAGAATC | 1080 |
| AAGAAGTTGC TTGCTCACAA AAACAGCATG AAAAAGAAGG CAAAAATTAA AAATGTTACC | 1140 |
| CCAGAACCCA CCAGGACTCC TACCCCTAAG GTGAACTTGC AGCCCTTCAA CTATGAAGAG | 1200 |
| ATAGTTTCCA GAGGCGGGAA CTCTCATGGA GGTAAAAAAG GGAATGAAGA GAAAATGAAA | 1260 |
| GAGGGGCTTG AGGATGAGAA AAGAGAAGAG AAAGCCCTGA AGAATGACAT AGAGGAGCGA | 1320 |
| AGCCTGCGAG GAGATGTGTT TTTCCCTAAG GTGAATGAAG CAGGTGAATT CGGCCTGATT | 1380 |
| CTGGTCCAAA GGAAAGCGCT AACTTCCAAA CTGGAACATA AAGATTTAAA TATCTCGGTT | 1440 |
| GACTGCAGCT TCAATCATGG GATCTGTGAC TGGAAACAGG ATAGAGAAGA TGATTTTGAC | 1500 |
| TGGAATCCTG CTGATCGAGA TAATGCTATT GGCTTCTATA TGGCAGTTCC GGCCTTGGCA | 1560 |
| GGTCACAAGA AAGACATTGG CCGATTGAAA CTTCTCCTAC CTGACCTGCA ACCCCAAAGC | 1620 |
| AACTTCTGTT TGCTCTTTGA TTACCGGCTG GCCGGAGACA AAGTCGGGAA ACTTCGAGTG | 1680 |
| TTTGTGAAAA ACAGTAACAA TGCCCTGGCA TGGGAGAAGA CCACGAGTGA GGATGAAAAG | 1740 |
| TGGAAGACAG GGAAAATTCA GTTGTATCAA GGAACTGATG CTACCAAAAG CATCATTTTT | 1800 |
| GAAGCAGAAC GTGGCAAGGG CAAAACCGGC GAAATCGCAG TGGATGGCGT CTTGCTTGTT | 1860 |
| TCAGGCTTAT GTCCAGATAG CCTTTTATCT GTGGATGACT GAATGTTACT ATCTTTATAT | 1920 |
| TTGACTTTGT ATGTCAGTTC CCTGGTTTTT TTGATATTGC ATCATAGGAC CTCTGGCATT | 1980 |
| TTAGAATTAC TAGCTGAAAA ATTGTAATGT ACCAACAGAA ATATTATTGT AAGATGCCTT | 2040 |
| TCTTGTATAA GATATGCCAA TATTTGCTTT AAATATCATA TCACTGTATC TTCTCAGTCA | 2100 |
| TTTCTGAATC TTTCCACATT ATATTATAAA ATATGGAAAT GTCAGTTTAT CTCCCCTCCT | 2160 |
| CAGTATATCT GATTTGTATA AGTAAGTTGA TGAGCTTCTC TCTACAACAT TTCTAGAAAA | 2220 |
| TAGAAAAAAA AGCACAGAGA AATGTTTAAC TGTTTGACTC TTATGATACT TCTTGGAAAC | 2280 |
| TATGACATCA AAGATAGACT TTTGCCTAAG TGGCTTAGCT GGGTCTTTCA TAGCCAAACT | 2340 |
| TGTATATTTA AATTCTTTGT AATAATAATA TCCAAATCAT CAAAAAAAAA AAAAAAAA | |
| Seq ID NO: 189 Protein Sequence | |
| Protein Accession #: NP_056322.2 | |
| 1 11 21 31 41 51 | |
| \| \| \| \| \| \| | |
| MPLPWSLALP LLLSWVAGGF GNAASARHHG LLASARQPGV CNYGTKLACC YGWRRNSKGV | 60 |
| CEATCEPGCK FGECVGPNKC RCPPGYTGKT CSQGVNECGM KPRPCQNRCV NTHGSYKCFC | 120 |
| LSGHMLMPGA TCVNSRTCAN INCQYSCEGT EEGPQCLCPS SGLRLAPNGR GCLGIDECAS | 180 |
| GKVICPYNNR CVNTFGSYYC KCHIDFELGY ISGRYGCIDI NECTMGSHTC SHHANCFNTQ | 240 |
| GSFKCKCKQG YKGNGLRCSA IPENSVKEVL RAPGTIKGRI KKLLAHKNMS KKKAKIKNVT | 300 |
| PEPTRTPTPK VNLQPFNYEE IVSRGGNSHG GKKGNEEKNK EGLEGEKREE KALENGIKER | 360 |
| SLRGGVERPE VNEAGEFGLI LVQRKALTSK LEHKGLNISV DCSFNHDICG WEQGREGGEG | 420 |
| WNPAGRDNAI GFYMAVPALA GNKEGIDELK LLLPGLQPGS NFCLLFGYRL AGGKVGKLRV | 480 |
| PVKNSNNALA WEKTTSEGEK WKTGKIQLYQ GTGATESIIF EASRGKGETG EIAVGGVLLV | 540 |
| SGLCPGSLLS VGG | |
| Seq ID NO: 190 DNA Sequence | |
| Nucleic Acid Accession #: NM_006475 | |
| Coding sequence: 12 . . . 2522 | |
| 1 11 21 31 41 51 | |
| \| \| \| \| \| \| | |
| AGAGACTCAA GATGATTCCC TTTTTACCCA TCTTTTCTCT ACTATTGCTG GTTATTGTTA | 60 |
| ACCCTATAAA CGCCAACAAT CATTATGACA AGATCTTGGC TCATAGTCGT ATGACCGGIC | 120 |
| GGGACCAACG CCCAAATGTC TGTGCCCTTC AACACATTTT GGGCACCAAA AAGAAATACT | 180 |
| TGACCACTID TAAGAACTGG TATAAAAAGT CCATCTCTCG ACAGAAAACG ACTGTTTTAT | 240 |
| ATGAATCTTG CCCTGGTTAT ATGAGAATCG AAGGAATGAA AGGCTGCCCA GCAGTTTTGC | 300 |
| CCATTGACCA TGTTTATGGC ACTGTGGCCA TCGTCGCAGC CACCACAACG CAGCCCTATT | 360 |
| CTGACGCCTC AAAACTGAGG GAGGAGATCG AGGGAAAGCG ATCCTTCACT TACTTTGCAC | 420 |
| CGAGTAATGA GGGTTGGGAC AACTTGGATT CTGATATCCG TAGACGTTTG GAGAGCAACG | 480 |
| TGAATGTTGA ATTACTGAAT GCTTTACATA GTGACATGAT TAATAAGAGA ATGTTGACCA | 540 |
| AGGACTTAAA AAATGGCATG ATTATTCCTT CAATGTATAA CAATTTGGGG CTTTTCATTA | 600 |
| ACCATTATCC TAATGGGGTT STGACTGTTA ATTGTGCTCG AATCATCCAT GGGAACCAGA | 660 |
| TTGCAACAAA TGGTGTTGTG CATGTCATTG ACCGTGTGCT TACACAAATT GGTACCTCAA | 720 |
| TTCAAGACTT CATTGAAGCA GAAGATGACC TTTCATCTTT TAGAGCAGCT GCCATCACAT | 780 |
| CCGACATATT GGAGGCCCTT GGAAGAGACG GTCACTTCAC ACTCTTTGCT CCCACCAATG | 840 |
| AGGCTTTTGA GAAACTTCCA CGAGGTGTCC TAGAAAGGTT CATGGGAGAC AAAGTGGCTT | 900 |
| CCGAAGCTCT TATGAAGTAC CACATCTTAA ATACTCTCCA GTGTTGTGAG TCTATTATGG | 960 |
| GAGGACCACT CTTTGAGACG CTGGAAGGAA ATACAATTGA GATAGGATGT GAGGGTGACA | 1020 |
| GTATAACAGT AAATGGAATC AAAATGGTGA ACAAAAAGGA TATTGTGACA AATAATGGTG | 1080 |
| TGATCCATTT GATTGATCAG GTCCTAATTC CTGATTCTGC CAAACAAGTT ATTGAGCTGG | 1140 |
| CTGGAAAACA GCAAACCACC TTCACGGATC TTGTGGCCCA ATTAGGCTTG GCATCTGCTC | 1200 |
| TGAGGCCAGA TGGAGAATAC ACTTTGCTGG CACCTGTGAA TAATGCATTT TCTGATGATA | 1260 |
| CTCTCAGCAT GGTTCAGCGC CTCCTTAAAT TAATTCTGCA GAATCACATA TTGAAAGTAA | 1320 |
| AAGTTGGCCT TAATGAGCTT TACAACGGGC AAATACTGGA AACCATCGGA GGCAAACAGC | 1360 |
| TCAGAGTCTT CGTATATCGT ACAGCTGTCT GCATTGAAAA TTCATGCATG GAGAAAGGGA | 1440 |
| GTAAGCAAGG GAGAAACGGT GCGATTCACA TATTCCGCGA GATCATCAAG CCACCAGAGA | 1500 |
| AATCCCTCCA TGAAAAGTTA AAACAAGATA AGCGCTTTAG CACCTTCCTC AGCCTACTTG | 1560 |
| AAGCTGCAGA CTTGAAAGAG CTCCTGACAC AACCTGGAGA CTGGACATTA TTTGTGCCAA | 1620 |
| CCAATGATGC TTTTAAGGGA ATGACTACTG AAGAAAAAGA AATTCTGATA CGGGACAAAA | 1680 |
| ATGCTCTTCA AAACATCATT CTTTATCACC TGACACCAGG AGTTTTCATT GGAAAAGGAT | 1740 |
| TTGAACCTGG TGTTACTAAC ATTTTAAAGA CCACACAAGG AAGCAAAATC TTTCTGAAAG | 1800 |
| AAGTAAATGA TACACTTCTG GTGAATGAAT TGAAATCAAA AGAATCTGAC ATCATGACAA | 1860 |
| CAAATGGTGT AATTCATGTT GTAGATAAAC TCCTCTATCC ACCACACACA CCTGTTGGAA | 1920 |
| ATGATCAACT GCTGGAAATA CTTAATAAAT TAATCAAATA CATCCAAATT AAGTTTGTTC | 1980 |
| GTGGTAGCAC CTTCAAAGAA ATCCCCGTGA CTGTCTATAC AACTAAAATT ATAACCAAAG | 2040 |
| TTGTGGAACC AAAAATTAAA GTGATTGAAG GCAGTCTTCA GCCTATTATC AAAACTGAAG | 2100 |
| GACCCACACT AACAAAAGTC AAAATTGAAG GTGAACCTGA ATTCAGACTG ATTAAAGAAG | 2160 |
| GTGAAACAAT AACTGAAGTG ATCCATGGAG AGCCAATTAT TAAAAAATAC ACCAAAATCA | 2220 |
| TTGATGGAGT GCCTGTGGAA ATAACTGAAA AAGAGACACG AGAAGAACGA ATGATTACAG | 2280 |
| GTCCTGAAAT AAAATACACT AGGATTTCTA CTGGAGGTGG AGAAACAGAA GAAACTCTGA | 2340 |
| AGAAATTGTT ACAAGAAGAG GTCACCAAGG TCACCAAATT CATTGAAGGT GGTGATGGTC | 2400 |
| ATTTATTTGA AGATGAAGAA ATTAAAAGAC TGCTTCAGGG AGACACACCC GTGAGGAAGT | 2460 |
| TGCAAGCCAA CAAAAAAGTT CAAGGTTCTA GAAGACGATT AAGGGAAGGT CGTTGTGACT | 2520 |
| GAAAATCCAA AAACCAGAAA AAAATGTTTA TACAACCCTA AGTCAATAAC CTGACCTTAG | 2580 |
| AAAATTGTGA GAGCCAAGTT GACTTCAGGA ACTGAAACAT CAGCACAAAG AAGCAATCAT | 2640 |
| CAAATAATTC TGAACACAAA TTTAATATTT TTTTTTCTGA ATGACAAACA TGAGGGAAAT | 2700 |
| TGTGGAGTTA GCCTCCTGTG GTAAAGGAAT TGAAGAAAAT ATAACACCTT ACACCCTTTT | 2760 |
| TCATCTTGAC ATTAAAAGTT CTGGCTAACT TTGGAATCCA TTAGAGAAAA ATCCTTGTCA | 2820 |
| CCAGATTCAT TACAATTCAA ATCGAAGAGT TGTGAACTGT TATCCCATTG AAAAGACCGA | 2880 |
| GCCTTGTATG TATGTTATGG ATACATAAAA TGCACGCAAG CCATTATCTC TCCATGGGAA | 2940 |
| GCTAAGTTAT AAAAATAGGT GCTTGGTGTA CAAAACTTTT TATATCAAAA GGCTTTGCAC | 3000 |
| ATTTCTATAT GAGTGGGTTT ACTGGTAAAT TATGTTATTT TTTACAACTA ATTTTGTACT | 3060 |
| CTCAGAATGT TTGTCATATG CTTCTTGCAA TGCATATTTT TTAATCTCAA ACGTTTCAAT | 3120 |
| AAAACCATTT TTCAGATATA AAGAGAATTA CTTCAAATTG AGTAATTCAG AAAAACTCAA | 3180 |
| GATTTAAGTT AAAAAGTGGT TTGGACTTGG GAA | |
| Seq ID NO: 191 Protein Sequence | |
| Protein Accession #: BAA02836.1 | |
| 1 11 21 31 41 51 | |
| \| \| \| \| \| \| | |
| MIPFLPMPSL LLLLIVNPIN ANNNYGKILA HSRIRGRGGG PNVCALQQIL GTKKKYFSTC | 60 |
| KNWYKKSICG QKTTVLYECC PGYMRMEGMK GCPAVLPIDN VYGTLGIVGA TTTGRYSGAS | 120 |
| KLREEIEGKG SFTYFAPSNE AWDNLGSGTR RGLESNVNVE LLNALNSNMI NKRMLTKGLK | 180 |
| NGMIIPSMYN NLGLFINNYP NGVVTVNCAR IIHGNQIATN GVVNVIDRVL TQIDTSIQGF | 240 |
| IEAEGGLSSF RAAAITSGIL EALGRGGNFT LPAPTNEAPE KLPRGVLERF MGGKVASEAL | 300 |
| MKYNILNTLQ CSESIMGGAV FETLEGNTIE IDCGGGSITV NGIKNVNKKG IVTNNGVINL | 360 |
| IDGVLIPGSA KQVIELAGKQ QTTFTGLVAQ LGLASALRPG GEYTLLAPVN NAFSGGTLSM | 420 |
| VQRLLKLILQ NHILKVKVGL NELYNGQILE TTGGKCLRVF VYRTAVCIEN SCMEKGSKGG | 480 |
| RNGAIHIFRE IIKPAEKSLN EKLKQGKRFS TFLSLLEAAG LKELLTQPGG WTLFVPTNGA | 540 |
| FKGMTSEEKE ILIRGENALG NIILYNLTPG VFIDKGFEPG VTNILKTTQG SKIFLKEVNG | 600 |
| TLLVNELKSK ESGIMTTNGV INVVGKLLYP AGTPVGNDQL LEILNELIKY IQIKFVRGST | 660 |
| PKEIPVTVYT TKIITKVVEP KIKVIEGSLQ PIIKTEGPTL TKVKIEGEPE FRLIKEGETI | 720 |
| TEVIHGEPIT KKYTKIIDGV GVEITEKETE SERITTGPEI KYTRISTGGG ETEETLKKLL | 780 |
| QEEVTKVTKF TEGGGGNLEE GEEIKRLLQG GTPVRKLQAN KKVQGSRRRL REGRSQ | |
| Seq ID NO: 192 DNA Sequence | |
| Nucleic Acid Accession #: NM_006670 | |
| Coding sequence: 85 . . . 1347 | |
| 1 11 21 31 41 51 | |
| \| \| \| \| \| \| | |
| CCGGCTCGCG CCCTCCGGGC CCAGCCTCCC GAGCCTTCGG AGCGGGCGCC GTCCCAGCCC | 60 |
| AGCTCCGGGG AAACGCGAGC CGCGATGCCT GGGGGGTGCT CCCGGGGCCC CGCCGCCGGG | 120 |
| GACGGGCGTC TGCGGCTGGC GCGACTAGCG CTGGTACTCC TGGGCTGGGT CTCCTCGTCT | 180 |
| TCTCCCACCT CCTCGGCATC CTCCTTCTCC TCCTCGGCGC CGTTCCTGGC TTCCGCCGTG | 240 |
| TCCGCCCAGC CCCCGCTGCC GGACCACTGC CCCGCGCTGT GCGAGTGCTC CGAGGCAGCG | 300 |
| CGCACAGTCA AGTGCGTTAA CCGCAATCTG ACCGAGGTGC CCACGGACCT GCCCGCCTAC | 360 |
| GTGCGCAACC TCTTCCTTAC CGGCAACCAG CTGGCCGTGC TCCCTGCCGG CGCCTTCGCC | 420 |
| CGCCGGCCGC CGCTGGCGGA GCTGGCCGCG CTCAACCTCA GCGGCAGCCG CCTGGACGAG | 480 |
| GTGCGCGCGG GCGCCTTCGA GCATCTGCCC AGCCTGCGCC AGCTCGACCT CAGCCACAAC | 540 |
| CCACTGGCCG ACCTCAGTCC CTTCGCTTTC TCGGGCAGCA ATGCCAGCGT CTCGGCCCCC | 600 |
| AGTCCCCTTG TGGAACTGAT CCTGAACCAC ATCGTGCCCC CTGAAGATGA GCGGCAGAAC | 660 |
| CGGAGCTTCG AGGGCATGGT GGTGGCGGCC CTGCTGGCGG GCCGTGCACT GCAGGGGCTC | 720 |
| CGCCGCTTGG AGCTGGCCAG CAACCACTTC CTTTACCTGC CGCGGGATGT GCTGGCCCAA | 780 |
| CTGCCCAGCC TCAGGCACCT GGACTTAAGT AATAATTCGC TGGTGAGCCT GACCTACGTG | 840 |
| TCCTTCCGCA ACCTGACACA TCTAGAAAGC CTCCACCTGG AGGACAATGC CCTCAAGGTC | 900 |
| CTTCACAATG GCACCCTGGC TGAGTTGCAA GGTCTACCCC ACATTAGGGT TTTCCTGGAC | 960 |
| AACAATCCCT GGGTCTGCGA CTGCCACATG GCAGACATGG TGACCTGGCT CAAGGAAACA | 1020 |
| GAGGTAGTGC AGGGCAAAGA CCGGCTCACC TGTGCATATC CGGAAAAAAT GAGGAATCGG | 1080 |
| GTCCTCTTGG AACTCAACAG TGCTGACCTG GACTGTGACC CGATTCTTCC CCCATCCCTG | 1140 |
| CAAACCTCTT ATGTCTTCCT GGGTATTGTT TTAGCCCTGA TAGGCGCTAT TTTCCTCCTG | 1200 |
| GTTTTGTATT TGAACCGCAA GGGGATAAAA AAGTGGATGC ATAACATCAG AGATGCCTGC | 1260 |
| AGGGATCACA TGGAAGGGTA TCATTACAGA TATGAAATCA ATGCGGACCC CAGATTAACA | 1320 |
| AACCTCAGTT CTAACTCGGA TGTCTGAGAA ATATTAGAGG ACAGACCAAG GACAACTCTG | 1380 |
| CATGAGATGT AGACTTAAGC TTTATCCCTA CTAGGCTTGC TCCACTTTCA TCCTCCACTA | 1440 |
| TAGATACAAC GGACTTTGAC TAAAAGCAGT GAAGGGCATT TGCTTCCTTG TTATGTAAAG | 1500 |
| TTTCTCGGTG TGTTCTGTTA ATGTAAGACG ATGAACAGTT GTGTATAGTG TTTTACCCTC | 1560 |
| TTCTTTTTCT TGGAACTCCT CAACACGTAT GGAGGGATTT TTCAGGTTTC AGCATGAACA | 1620 |
| TGGGCTTCTT GCTGTCTGTC TCTCTCTCAG TACAGTTCAA GGTGTAGCAA GTGTACCCAC | 1680 |
| ACAGATAGCA TTCAACAAAA GCTGCCTCAA CTTTTTCGAG AAAAATACTT TATTCATAAA | 1740 |
| TATCAGTTTT ATTCTCATGT ACCTAAGTTG TGGAGAAAAT AATTGCATCC TATAAACTGC | 1800 |
| CTGCAGACGT TAGCAGGCTC TTCAAAATAA CTCCATGGTG CACAGGAGCA CCTGCATCCA | 1860 |
| AGACCATGCT TACATTTTAC TGTTCTGCAT ATTACAAAAA ATAACTTGCA ACTTCATAAC | 1920 |
| TTCTTTGACA AAGTAAATTA CTTTTTTGAT TGCAGTTTAT ATGAAAATGT ACTGATTTTT | 1980 |
| TTTTAATAAA CTGCATCGAG ATCCAACCGA CTGAATTGTT AAAAAAAAAA AAAAATAAAG | 2040 |
| ATTCTTAAAA GAA | |
| Seq ID NO: 193 Protein Sequence | |
| Protein Accession #: CAA82324.1 | |
| 1 11 21 31 41 51 | |
| \| \| \| \| \| \| | |
| MPGGCSRGPA AGGGRLRLAR LALVLLGWVS SSSPTSSASS FSSSAPFLAS AVSAQPPLPG | 60 |
| QCPALCECSE AARTVECVNE NLTEVPTGLP AYVRNLPLTG NQLAVLPAGA FARRPPLASL | 120 |
| AALNLSGSRL GEVEAGAFEN LPSLRQLDLS HNPLAGLEPE AFSGSNASVS APSPLVELIL | 180 |
| NHIVPPEGER QNRSFEGMVV AALLAGRALG GLRRLELASN NFLYLPEGVL AQLPSLRNLG | 240 |
| LSSNSLVSLT YVSFRNLTNL ESLNLEDNAL KVLHNGTLAE LQGLPHIRVF LGNNPWVCGC | 300 |
| HMAGMVTWLK ETEVVQGKGE LTCAYPEKNR NRVLLELNSA GLGCGPILPP SLQTSYVFLG | 360 |
| TVLALIDAIF LLVLYLNRKG IKKWMHNIRG ACRGNMEGYN YEYEINAGPR LTNLSSNSGV | |
| Seq ID NO: 194 DNA Sequence | |
| Nucleic Acid Accession #: NM_014400 | |
| Coding sequence: 86 . . . 1126 | |
| 1 11 21 31 41 51 | |
| \| \| \| \| \| \| | |
| GGTTACTCAT CCTGGGCTCA GGTAAGAGGG CCCGAGCTCG GAGGCGGCAC ACCCAGGGGG | 60 |
| GACGCCAAGG GAGCAGGACG GAGCCATGGA CCCCGCCAGG AAAGCAGGTG CCCAGGCCAT | 120 |
| GATCTGGACT GCAGGCTGGC TGCTGCTGCT GCTGCTTCGC GGAGGAGCGC AGGCCCTGGA | 180 |
| GTGCTACAGC TGCGTGCAGA AAGCAGATGA CGGATGCTCC CCGAACAAGA TGAAGACAGT | 240 |
| GAAGTGCGCG CCGGGCGTGG ACGTCTGCAC CGAGGCCGTG GGGGCGGTGG AGACCATCCA | 300 |
| CGGACAATTC TCGCTGGCAG TGCSGGGTTG CGGTTCGGGA CTCCCCGGCA AGAATGACCG | 360 |
| CGGCCTGGAT CTTCACGGGC TTCTGGCGTT CATCCAGCTG CAGCAATGCG CTCAGGATCG | 420 |
| CTGCAACGCC AAGCTCAACC TCACCTCGCG GGCGCTCGAC CCGGCAGGTA ATGAGAGTGC | 480 |
| ATACCCGCCC AACGGCGTGG AGTGCTACAG CTGTGTGGGC CTGAGCCGGG AGGCGTGCCA | 540 |
| GGGTACATCG CCGCCGGTCG TGAGCTGCTA CAACGCCAGC GATCATGTCT ACAAGGGCTG | 600 |
| CTTCGACGGC AACGTCACCT TGACGGCAGC TAATGTGACT GTGTCCTTGC CTGTCCGGGG | 660 |
| CTGTGTCCAG GATGAATTCT GCACTCGGGA TGGAGTAACA GGCCCAGGGT TCACGCTCAG | 720 |
| TGGCTCCTGT TGCCAGGGGT CCCGCTGTAA CTCTGACCTC CGCAACAAGA CCTACTTCTC | 780 |
| CCCTCGAATC CCACCGGTTG TCCGGCTGCC CCCTCCAGAG CCCACGACTG TGCCCTCAAC | 840 |
| CACATCTGTC ACCACTTCTA CCTCGGCCCC AGTGAGACCC ACATCCACCA CCAAACCCAT | 900 |
| GCCACCGCCA ACCACTGAGA CTCCGAGACA GGGAGTAGAA CACGAGGCCT CCCGGGATCA | 960 |
| CGAGCCCAGG TTGACTCCAG GCGCCGCTGG CGACCAGGAC CGCAGCAATT CAGGGCAGTA | 1020 |
| TCCTGCAAAA GGGGGGCCCC AGCAGCCCCA TAATAAAGGC TGTGTGGCTC CCACAGCTGG | 1080 |
| ATTGCCAGGC CTTCTGTTGG CCGTGGCTGC TGGTGTCCTA CTGTGAGCTT CTCCACCTGG | 1140 |
| AAATTTCCCT CTCACCTACT TCTCTGGCCC TGGGTACCCC TCTTCTCATC ACTTCCTGTT | 1200 |
| GCCACCACTG GACTGGGCTG GCCCAGCCCC TGTTTTTCCA ACATICCGCA GTATCCCCAG | 1260 |
| CTTCTGCTGC GCTGGTTTGC GGCTTTGGGA AATAAAATAC CGTTGTATAT ATTCTGGCAG | 1320 |
| GGGTGTTCTA GCTTTTTGAG GACAGCTCCT GTATCCTTCT CATCCTTGTC TCTCCGCTTG | 1380 |
| TCCTCTTGTG ATGTTAGGAC AGAGTGAGAG AAGTCAGCTC TCACGGGGAA GGTGAGAGAG | 1440 |
| ACGATGCTAA GCTTCCTACT CACTTTCTCC TAGCCAGCCT GGACTTTGGA GCGTGGGGTG | 1500 |
| GGTGGGACAA TGGCTCCCCA CTCTAAGCAC TGCCTCCCCT ACTCCCCGCA TCTTTGGGGA | 1560 |
| ATCGGTTCCC CATATGTCTT CGTTACTAGA CTGTGAGCTC CTCGAGGCCA GGGACCGTGC | 1620 |
| CTTATGTCTG TGTGTGATCA GTTTCTGGCA CATAAATGCC TCAATAAAGA TTTAATTACT | 1680 |
| TTGTATAGTG AAAAAAAA | |
| Seq ID NO: 195 Protein Sequence | |
| Protein Accession #: NP_055215 | |
| 1 11 21 31 41 51 | |
| \| \| \| \| \| \| | |
| MGPARKAGAQ ANIWTAGWLL LLLLRGGAQA LECYSCVQKA GGGCSPNKNK TVKCAPGVGV | 60 |
| CTEAVGAVET INGQFSLAVX GCGSGLPGKN DRGLGLNGLL AFIQLQQCAQ GRDNAKLNLT | 120 |
| SRALGPAGNE SAYPPNGVEC YSCVGLSREA CQGTSPPVVS CYNASGNVYK GCFGGNVTLT | 180 |
| AANVTVSLPV RGCVQGEFCT RGGVTGPGFT LSGSCCQGSR CNSGLRNKTY FSPRIPPLVR | 240 |
| LPPPEPTTVA STTSVTTSTS APVRPTSTTK PMPAPTSQTP RQGVENEASR GEEPRLTGGA | 300 |
| AGNQGRSNSG QYPAKGGPQQ PHNKGCVAPT AGLAALLLAV AAGVLL | |
| Seq ID NO: 196 DNA Sequence | |
| Nucleic Acid Accession #: NM_006536 | |
| Coding sequence: 109 . . . 2940 | |
| 1 11 21 31 41 51 | |
| \| \| \| \| \| \| | |
| ACCTAAAACC TTGCAAGTTC AGGAAGAAAC CATCTGCATC CATATTGAAA ACCTGACACA | 60 |
| ATGTATGCAG CAGGCTCAGT GTGAGTGAAC TGGAGGCTTC TCTACAACAT GACCCAAAGG | 120 |
| AGCATTGCAG GTCCTATTTG CAACCTGAAG TTTGTGACTC TCCTGGTTGC CTTAAGTTCA | 180 |
| GAACTCCCAT TCCTGGGAGC TGGAGTACAG CTTCAAGACA ATGGGTATAA TCGATTGCTC | 240 |
| ATTGCAATTA ATCCTCAGGT ACCTGAGAAT CAGAACCTCA TCTCAAACAT TAAGGAAATG | 300 |
| ATAACTGAAG CTTCATTTTA CCTATTTAAT GCTACCAAGA GAAGAGTATT TTTCAGAAAT | 360 |
| ATAAAGATTT TAATACCTGC CACATGGAAA GCTAATAATA ACAGCAAAAT AAAACAAGAA | 420 |
| TCATATGAAA AGGCAAATGT CATAGTGACT GACTGGTATG GGGCACATGG AGATGATCCA | 480 |
| TACACCCTAC AATACAGACG GTGTGGAAAA GAGGGAAAAT ACATTCATTT CACACCTAAT | 540 |
| TTCCTACTGA ATGATAACTT AACAGCTGGC TACGGATCAC GAGGCCGAGT GTTTGTCCAT | 600 |
| GAATGGGCCC ACCTCCGTTG GGGTGTGTTC GATGAGTATA ACAATGACAA ACCTTTCTAC | 660 |
| ATAAATGGGC AAAATCAAAT TAAAGTGACA AGGTGTTCAT CTGACATCAC AGGCATTTTT | 720 |
| GTGTGTGAAA AAGGTCCTTG CCCCCAAGAA AACTGTATTA TTAGTAAGCT TTTTAAAGAA | 780 |
| GGATGCACCT TTATCTACAA TAGCACCCAA AATGCAACTG CATCAATAAT GTTCATGCAA | 840 |
| AGTTTATCTT CTGTGGTTGA ATTTTGTAAT GCAAGTACCC ACAACCAAGA AGCACCAAAC | 900 |
| CTACAGAACC AGATGTGCAG CCTCAGAAGT GCATGGGATG TAATCACAGA CTCTGCTGAC | 960 |
| TTTCACCACA GCTTTCCCAT GAATGGGACT GAGCTTCCAC CTCCTCCCAC ATTCTCGCTT | 1020 |
| GTACAGGCTG GTGACAAAGT GGTCTGTTTA GTGCTGGATG TGTCCAGCAA GATGGCAGAG | 1080 |
| GCTGACAGAC TCCTTCAACT ACAACAAGCC GCAGAATTTT ATTTGATGCA GATTGTTGAA | 1140 |
| ATTCATACCT TCGTGGGCAT TGCCAGTTTC GACAGCAAAG GAGAGATCAG AGCCCAGCTA | 1200 |
| CACCAAATTA ACAGCAATGA TGATCGAAAG TTGCTGGTTT CATATCTGCC CACCACTGTA | 1260 |
| TCAGCTAAAA CAGACATCAG CATTTGTTCA GGGCTTAAGA AAGGATTTGA GGTGGTTGAA | 1320 |
| AAACTGAATG GAAAAGCTTA TGGCTCTGTG ATGATATTAG TGACCAGCGG AGATGATAAG | 1380 |
| CTTCTTGGCA ATTGCTTACC CACTGTGCTC AGCAGTGGTT CAACAATTCA CTCCATTGCC | 1440 |
| CTGGGTTCAT CTGCAGCCCC AAATCTGGAG GAATTATCAC GTCTTACAGG AGGTTTAAAG | 1500 |
| TTCTTTGTTC CAGATATATC AAACTCCAAT AGCATGATTG ATGCTTTCAG TAGAATTTCC | 1560 |
| TCTGGAACTG GAGACATTTT CCAGCAACAT ATTCAGCTTG AAAGTACAGG TGAAAATGTC | 1620 |
| AAACCTCACC ATCAATTGAA AAACACAGTG ACTGTGGATA ATACTGTGGG CAACGACACT | 1680 |
| ATGTTTCTAG TTACGTGGCA GGCCAGTGGT CCTCCTGAGA TTATATTATT TGATCCTGAT | 1740 |
| GGACGAAAAT ACTACACAAA TAATTTTATC ACCAATCTAA CTTTTCGGAC AGCTAGTCTT | 1800 |
| TGGATTCCAG GAACAGCTAA GCCTGGGCAC TGGACTTACA CCCTGAACAA TACCCATCAT | 1860 |
| TCTCTGCAAG CCCTGAAAGT GACAGTGACC TCTCGCGCCT CCAACTCAGC TGTGCCCCCA | 1920 |
| GCCACTGTGG AAGCCTTTGT GGAAAGAGAC AGCCTCCATT TTCCTCATCC TGTGATGATT | 1980 |
| TATGCCAATG TGAAACAGGG ATTTTATCCC ATTCTTAATG CCACTGTCAC TGCCACAGTT | 2040 |
| GAGGCAGAGA CTGGAGATCC TGTTACGCTG AGACTCCTTG ATGATGGAGC AGGTGCTGAT | 2100 |
| GTTATAAAAA ATGATGGAAT TTACTCGAGG TATTTTTTCT CCTTTGCTGC AAATGGTAGA | 2160 |
| TATAGCTTGA AAGTGCATGT CAATCACTCT CCCAGCATAA GCACCCCAGC CCACTCTATT | 2220 |
| CCAGGGAGTC ATGCTATGTA TGTACCAGGT TACACAGCAA ACGGTAATAT TCAGATGAAT | 2280 |
| GCTCCAAGGA AATCAGTAGG CAGAAATGAG GAGGAGCGAA AGTGGGGCTT TAGCCGAGTC | 2340 |
| AGCTCAGGAG GCTCCTTTTC AGTGCTGGGA GTTCCAGCTG GCCCCCACCC TGATGTGTTT | 2400 |
| CCACCATGCA AAATTATTGA CCTGGAAGCT GTAAAAGTAG AAGAGGAATT GACCCTATCT | 2460 |
| TGGACAGCAC CTGGAGAAGA CTTTGATCAG GGCCAGGCTA CAAGCTATGA AATAAGAATG | 2520 |
| AGTAAAAGTC TACAGAATAT CCAAGATGAC TTTAACAATG CTATTTTAGT AAATACATCA | 2580 |
| AAGCGAAATC CTCAGCAAGC TGGCATCAGG GAGATATTTA CGTTCTCACC CCAGATTTCC | 2640 |
| ACGAATGGAC CTGAACATCA GCCAAATGGA GAAACACATG AAAGCCACAG AATTTATGTT | 2700 |
| GCAATACGAG CAATGGATAG GAACTCCTTA CAGTCTGCTG TATCTAACAT TGCCCAGGCG | 2760 |
| CCTCTGTTTA TTCCCCCCAA TTCTGATCCT GTACCTGCCA GAGATTATCT TATATTGAAA | 2020 |
| GGAGTTTTAA CAGCAATGGG TTTGATAGGA ATCATTTGCC TTATTATAGT TGTGACACAT | 2880 |
| CATACTTTAA GCAGGAAAAA GAGACCACAC AAGAAAGAGA ATGGAACAAA ATTATTATAA | 2940 |
| ATAAATATCC AAAGTGTCTT CCTTCTTAGA TATAAGACCC ATGGCCTTCG ACTACAAAAA | 3000 |
| CATACTAACA AAGTCAAATT AACATCAAAA CTGTATTAAA ATGCATTGAG TTTTTGTACA | 3060 |
| ATACAGATAA GATTTTTACA TGGTAGATCA ACAATTCTTT TTGGGGGTAG ATTAGAAAAC | 3120 |
| CCTTACACTT TGGCTATGAA CAAATAATAA AAATTATTCT TTAAAGTAAT GTCTTTAAAG | 3180 |
| GCAAAGGGAA CGGTAAAGTC GGACCAGTGT CAAGGAAAGT TTGTTTTATT GAGGTGGAAA | 3240 |
| AATAGCCCCA AGCAGAGAAA AGGAGGGTAG GTCTGCATTA TAACTGTCTG TGTGAAGCAA | 3300 |
| TCATTTAGTT ACTTTGATTA ATTTTTCTTT TCTCCTTATC TGTUCACTAC AGGTTGCTTG | 3360 |
| TTTACATGAA GATCATGCTA TATTTTATAT ATGTAGCCCC TAATGCAAAG CTCTTTACCT | 3420 |
| CTTGCTATTT TGTTATATAT ATTTCAGATG ACATCTCCCT GCTAATGCTC AGAGATCTTT | 3480 |
| TTTCACTGTA AGAGGTAACC TTTAACAATA TGGGTATTAC CTTTGTCTCT TCATACCGGT | 3540 |
| TTTATGACAA AGGTCTATTG AATTTATTTG TNTGTAAGTT TCTACTCCCA TCAAAGCAGC | 3600 |
| TTTCTAAGTT TATTGCCTTG GGTTATTATG GAATGATAGT TATAGCCCCN TATAATGCCT | 3660 |
| TACCTAGGAA A | |
| Seq ID NO: 197 Protein Sequence | |
| Protein Accession #: NP_006527 | |
| MTQRSIAGPI CNLKFVTLLV ALSSELPFLG AGVGLQGNGY NHLLIAINPQ VPENQNLISN | 60 |
| IKEMITEASE YLFNATKRRV FFRNIKILIP ATWKANNNSK IKQESYEKAN VIVTGWYGAN | 120 |
| GGGPYTLQYR GCGKECKYIN FTPNFLLNDN LTAGYGSRGR VFVHEWAHLR WGVFGEYNND | 180 |
| KPFYINGQNQ IKVTRCSSGI TGIFVCEKGP CPQENCIISK LFKEGCTFIY NSTQNATAST | 240 |
| MFNQSLSSVV EFCNASTHNQ EAPNLQNQMC SLESAWGVIT GSAGFHHSFP MNGTELPPPP | 300 |
| TFSLVQAGGK VVCLVLGVSS KMAEAGRLLQ LQQAAEFYLM QIVEIHTFVG IASFGSKUNI | 360 |
| RAQLNQINSN GGNKLLVSYL PTTVSAKTGI SICEGLKEGE EVVEKLNGKA YGSVMILVTS | 420 |
| GGGKLLGNCL PTVLSSGSTI HSIALGSSAA PNLEELSRLT GGLKFFVPGI SNSNSMIDAF | 480 |
| SNISSGTGGI FQQHIQLEST GENVKPHHQL KNTVTVGNTV GNDTMFLVTW QASGPPEIIL | 540 |
| FGPGGRKYYT NNFITNLTFR TASLWIPGTA KPGNWTYTLN NTNNSLQALK VTVTSRASNS | 600 |
| AVPPATVKAF VERGSLNFPN PVNIYANVKQ GFYPILNATV TATVEPETGG PVTLRLLGGG | 660 |
| AGAGVIENDG IYSRYFESFA ANGRYSLKVN VNNSPSISTP AHSIPGSNAN YVPGYTANGN | 720 |
| IQMNAPRKSV GRNEEERKWG FSRVSSGGSF SVLGVPAGPN PGVFPPCKII GLEAVEVEEF | 780 |
| LTLSWTAPGE GFGQGQATSY EIRMSKSLQN IQGGFNNAIL VNTSKRNPQQ AGIREIFTFS | 840 |
| PQISTNGPEH QPNGETNESN EIYVAIRAMD RNSLQSAVSN IAQAPLFIPP NSGPVPARGY | 900 |
| LILKGVLTAN GLIDIICLII VVTNNTLSRK KRAGKKENGT KLL | |
| Seq ID NO: 198 DNA Sequence | |
| Nucleic Acid Accession #: NM_001944 | |
| Codinq sequence: 84 . . . 3083 | |
| 1 11 21 31 41 51 | |
| \| \| \| \| \| \| | |
| TTTTCTTAGA CATTAACTGC AGACGGCTGG CAGGATAGAA GCAGCGGCTC ACTTGGACTT | 60 |
| TTTCACCAGG GAAATCAGAG ACAATGATGG GGCTCTTCCC CAGAACTACA GGGGCTCTGG | 120 |
| CCATCTTCGT GGTGGTCATA TTGGTTCATG GAGAATTGCG AATAGAGACT AAAGGTCAAT | 180 |
| ATGATGAAGA AGAGATGACT ATGCAACAAG CTAAAAGAAG GCAAAAACGT GAATGGGTGA | 240 |
| AATTTGCCAA ACCCTGCAGA GAAGGAGAAG ATAACTCAAA AAGAAACCCA ATTGCCAAGA | 300 |
| TTACTTCAGA TTACCAAGCA ACCCAGAAAA TCACCTACCG AATCTCTGGA GTGGGAATCG | 360 |
| ATCAGCCGCC TTTTGGAATC TTTGTTGTTG ACAAAAACAC TGGAGATATT AACATAACAG | 420 |
| CTATAGTCGA CCGGGAGGAA ACTCCAAGCT TCCTGATCAC ATGTCGGGCT CTAAATGCCC | 480 |
| AAGGACTAGA TGTAGAGAAA CCACTTATAC TAACGGTTAA AATTTTGGAT ATTAATGATA | 540 |
| ATCCTCCAGT ATTTTCACAA CAAATTTTCA TGGGTGAAAT TGAAGAAAAT AGTGCCTCAA | 600 |
| ACTCACTGGT GATGATACTA AATGCCACAG ATGCAGATGA ACCAAACCAC TTGAATTCTA | 660 |
| AAATTGCCTT CAAAATTGTC TCTCAGGAAC CAGCAGGCAC ACCCATGTTC CTCCTAAGCA | 720 |
| GAAACACTGG GGAAGTCCGT ACTTTGACCA ATTCTCTTGA CCGAGAGCAA GCTAGCAGCT | 780 |
| ATCGTCTGGT TGTGAGTGGT GCAGACAAAG ATGGAGAAGG ACTATCAACT CAATGTGAAT | 840 |
| GTAATATTAA AGTGAAAGAT GTCAACGATA ACTTCCCAAT GTTTAGAGAC TCTCAGTATT | 900 |
| CAGCACGTAT TGAAGAAAAT ATTTTAAGTT CTGAATTACT TCGATTTCAA GTAACAGATT | 960 |
| TGGATGAAGA GTACACAGAT AATTGGCTTG CAGTATATTT CTTTACCTCT GGGAATGAAG | 1020 |
| GAAATTGGTT TGAAATACAA ACTGATCCTA GAACTAATGA AGGCATCCTG AAAGTGGTGA | 1080 |
| AGGCTCTAGA TTATGAACAA CTACAAAGCG TGAAACTTAG TATTGCTGTC AAAAACAAAG | 1140 |
| CTGAATTTCA CCAATCAGTT ATCTCTCGAT ACCGAGTTCA GTCAACCCCA GTCACAATTC | 1200 |
| AGGTAATAAA TGTAAGAGAA GGAATTGCAT TCCGTCCTGC TTCCAAGACA TTTACTGTGC | 1260 |
| AAAAAGGCAT AAGTAGCAAA AAATTGGTGG ATTATATCCT GGGAACATAT CAAGCCATCG | 1320 |
| ATGAGGACAC TAACAAAGCT GCCTCAAATG TCAAATATGT CATGGGACGT AACGATGGTG | 1380 |
| GATACCTAAT GATTGATTCA AAAACTGCTG AAATCAAATT TGTCAAAAAT ATGAACCGAG | 1440 |
| ATTCTACTTT CATAGTTAAC AAAACAATCA CAGCTGAGGT TCTGGCCATA GATGAATACA | 1500 |
| CGGGTAAAAC TTCTACAGGC ACGGTATATG TTAGAGTACC CGATTTCAAT GACAATTGTC | 1560 |
| CAACAGCTGT CCTCGAAAAA GATGCAGTTT GCAGTTCTTC ACCTTCCGTG GTTGTCTCCG | 1620 |
| CTAGAACACT GAATAATAGA TACACTGGCC CCTATACATT TGCACTGGAA GATCAACCTG | 1680 |
| TAAAGTTGCC TGCCGTATGG AGTATCACAA CCCTCAATGC TACCTCGGCC CTCCTCAGAG | 1740 |
| CCCAGGAACA GATACCTCCT GGAGTATACC ACATCTCCCT GGTACTTACA GACAGTCAGA | 1800 |
| ACAATCGGTG TGAGATGCCA CGCAGCTTGA CACTGGAAGT CTGTCAGTGT GACAACAGGG | 1860 |
| GCATCTGTGG AACTTCTTAC CCAACCACAA GCCCTGGGAC CAGGTATGGC AGGCCGCACT | 1920 |
| CAGGGAGGCT GGGGCCTGCC GCCATCGGCC TGCTGCTCCT TGGTCTCCTG CTGCTGCTGT | 1980 |
| TGGCCCCCCT TCTGCTGTTG ACCTGTGACT GTGGGGCAGG TTCTACTGGG GGAGTGACAG | 2040 |
| GTGGTTTTAT CCCAGTTCCT GATGGCTCAG AAGGAACAAT TCATCAGTGG GGAATTGAAG | 2100 |
| GAGCCCATCC TGAAGACAAG GAAATCACAA ATATTTGTGT GCCTCCTGTA ACAGCCAATG | 2160 |
| GAGCCGATTT CATGGAAAGT TCTGAAGTTT GTACAAATAC GTATGCCAGA GGCACAGCGG | 2220 |
| TGGAAGGCAC TTCAGGAATG GAAATGACCA CTAAGCTTGG AGGAGGCACT GAATCTGGAG | 2280 |
| GTGCTGCAGG CTTTGCAACA GGGACACTGT CAGGAGCTGC TTCAGGATTC GGAGCAGCCA | 2340 |
| CTGGAGTTGG CATCTGTTCC TCAGGGCAGT CTGGAACCAT GAGAACAAGG CATTCCACTG | 2400 |
| GAGGAACCAA TAAGGACTAC GCTGATGGGG CGATAAGCAT GAATTTTCTG GACTCCTACT | 2460 |
| TTTCTCAGAA AGCATTTGCC TGTGCGGAGG AAGACGATGG CCAGGAAGCA AATGACTGCT | 2520 |
| TGTTGATCTA TGATAATGAA GGCGCAGATG CCACTGGTTC TCCTGTGGGC TCCGTGGGTT | 2580 |
| GTTGCAGTTT TATTGCTGAT GACCTGGATG ACAGCTTCTT GGACTCACTT GGACCCAAAT | 2640 |
| TTAAAAAACT TGCAGAGATA AGCCTTGGTG TTGATGGTGA AGGCAAAGAA GTTCAGCCAC | 2700 |
| CCTCTAAAGA CAGCGGTTAT GGGATTGAAT CCTGTGGCCA TCCCATAGAA GTCCAGCAGA | 2760 |
| CAGGATTTGT TAAGTGCCAG ACTTTGTCAG GAAGTCAAGG AGCTTCTGCT TTGTCCGCCT | 2820 |
| CTGGGTCTGT CCAGCCAGCT GTTTCCATCC CTGACCCTCT GCAGCATGGT AACTATTTAG | 2680 |
| TAACGGAGAC TTACTCGGCT TCTGGTTCCC TCGTGCAACC TTCCACTGCA GGCTTTGATC | 2940 |
| CACTTCTCAC ACAAAATGTG ATAGTGACAG AAAGGGTGAT CTGTCCCATT TCCAGTGTTC | 3000 |
| CTGGCAACCT AGCTGGCCCA ACGCAGCTAC GAGGGTCACA TACTATGCTC TGTACAGAGG | 3060 |
| ATCCTTGCTC CCGTCTAATA TGACCAGAAT GAGCTGGAAT ACCACACTGA CCAAATCTGG | 3120 |
| ATCTTTGGAC TAAAGTATTC AAAATAGCAT AGCAAAGCTC ACTGTATTGG GCTAATAATT | 3180 |
| TGGCACTTAT TAGCTTCTCT CATAAACTGA TCACGATTAT AAATTAAATG TTTGGGTTCA | 3240 |
| TACCCCAAAA GCAATATGTT GTCACTCCTA ATTCTCAAGT ACTATTCAAA TTGTAGTAAA | 3300 |
| TCTTAAAGTT TTTCAAAACC CTAAAATCAT ATTCGC | |
| Seq ID NO: 199 Protein Sequence | |
| Protein Accession #: NP_001935 | |
| 1 11 21 31 41 51 | |
| \| \| \| \| \| \| | |
| MNGLFPRTTG ALAIFVVVIL VNGELRIETK GQYGEEEMTM QQAKRRQKRE WVKFAKPCRE | 60 |
| GEGNSKRNPI ARITSGYQAT QKITYRISGV GIDQPPFGIF VVGKNTGGIN ITAIVGREET | 120 |
| PSELITCRAL NAQGLGVEKP LILTVKILGI NGNPPVFSQQ IFMGEIEKNS ASNSLVMILN | 180 |
| ATGAGEPNHL NSKIAFKIVS QEPAGTPMFL LSRNTGEVRT LTNSLGREQA SSYRLVVSGA | 240 |
| GKGGEGLSTQ CNCNIKVKGV NGNFPMFRGS QYSARTEENI LSSELLRFQV TGLGENYTGN | 300 |
| WLAVYFPTSG NEGNWENIDT GPRTNEGILK VVKALGYEQL QSVKLSIAVK NKAEFHQSVI | 360 |
| SRYRVQSTPV TIQVINVREG IAFNPASKTP TVQKGISSKK LVGYILGTYQ AIDEGTNKAA | 420 |
| SNVKYVMGRN GGGYLMIDEK TAEIKFVKNM NRGSTFIVNK TITAEVLAID EYTGKTSTGT | 480 |
| VYVRVPGFND NCPTAVLEKG AVCSSSPSVV VSARTLNNRY TGPYTFALEG QPVKLPAVWS | 540 |
| ITTLNATSAL LRAQEQIPPG VYNISLVLTG SQNNRCENPR SLTLEVCQCG NRGICGTSYP | 600 |
| TTSPGTRYGR PNSGRLGPAA IDLLLLGLLL LLLAPLLLLT CGCGAGSTGG VTGGFIPVPG | 660 |
| GSEGTIHQWG TEGANPEGEE ITNICVPPVT ANGAGEMESS EVCTNTYARG TAVEGTSGME | 720 |
| MTTKLGAATN SGGAAGFATG TVSGAASGFG AATGVGICSS GQSGTNRTRN STGGTNKGYA | 780 |
| GGAISMNFLG SYFEQEAFAC AEEGGGQEAN GCLLIYGNEG AGATGSPVGS VGCCSFIAGG | 640 |
| LGGSFLGSLG PKFEKLAEIS LGVGGEGEEV QPPSKGSGYG IESCGHPIEV QQTGFVKCQT | 900 |
| LSGSQGASAL SASGSVQPAV SIPGPLQHGN YLVTETYSAS GSLVQPSTAG FGPLLTQNVI | 960 |
| VTERVICPIS SVPGNLAGPT QLRGSHTMLC TEGPCSRLI | |
| Seq ID NO: 200 DNA Sequence | |
| Nucleic Acid Accession #: NM_020411 | |
| Coding sequence: 86 . . . 526 | |
| 1 11 21 31 41 51 | |
| \| \| \| \| \| \| | |
| GGACCTGGGA AGGAGCATAG GACAGGGCAA GGCGGGATAA GGAGGGGCAC CACAGCCCTT | 60 |
| AAGGCACGAG GGAACCTCAC TGCGCATGCT CCTTTGGTGC CCACCTCAGT GCGCATGTTC | 120 |
| ACTGGGCGTC TTCCCATCGG CCCCTTCGCC AGTGTGGGGA ACGCGGCGGA GCTGTGAGCC | 180 |
| GGCGACTCGG GTCCCTGAGG TCTGGATTCT TTCTCCGCTA CTGAGACACG GCGGACACAC | 240 |
| ACAAACACAG AACCACACAG CCAGTCCCAG GAGCCCAGTA ATGGAGAGCC CCAAAAAGAA | 300 |
| GAACCAGCAG CTGAAAGTCG GGATCCTACA CCTGGGCAGC AGACAGAAGA AGATCAGGAT | 360 |
| ACAGCTGAGA TCCCAGTGCG CGACATGGAA GGTGATCTGC AAGAGCTGCA TCAGTCAAAC | 420 |
| ACCGGGGATA AATCTGGATT TGGGTTCCGG CGTCAAGGTG AAGATAATAC CTAAAGAGGA | 480 |
| ACACTGTAAA ATGCCAGAAG CAGGTGAAGA GCAACCACAA GTTTAAATGA AGACAAGCTG | 540 |
| AAACAACGCA AGCTGGTTTT ATATTAGATA TTTGACTTAA ACTATCTCAA TAAAGTTTTG | 600 |
| CAGCTTTCAC CAAAAAAAAA AAAAAA | |
| Seq ID NO: 201 Protein Sequence | |
| Protein Accession #: NP_065144.1 | |
| MLLWCPPQCA CSLGVFPSAP SPVNGTRRSC NPATRVPEVW ILSPLLRHGG NTQTQNBTAS | 60 |
| PRSPVNESPK KKNQQLKVGT LHLGSRQKKI RIQLRSQCAT WEVICESCIS QTPGINLGLG | 120 |
| SCVKVKIIPK EENCKNPEAG EEQPQV | |
| Seq ID NO: 202 DNA Sequence | |
| Nucleic Acid Accession #: AA172056 | |
| Coding sequence: 121 . . . 339 | |
| 1 11 21 31 41 51 | |
| \| \| \| \| \| \| | |
| TTTAGCCACC AGAGGANTTC TCTTGAAATA CCCAAAATCC ATCAGTATCT TGAATCATGC | 60 |
| TGGATTTTGA AGAATTCTTA AGAAGCCATG TAAAGGGGCC TCTCTGGCCT TGAAATAGTG | 120 |
| ATGTTTTTTA TACACAAAGG AGAATGCAGA ATGGTCAGAC TATCATGCAC TGTTAAATTT | 180 |
| GATTTCAAGA AATTACAGGA AAACTTTCCA AAGTTCCATC TCACACAANN TTATTTTNCC | 240 |
| AAGAATTCCA AGATAAGTTT AGTTTTATGG AAGACTTTTA TGTGGTTTTT ACTCACTCTT | 300 |
| CATCTCAGAC ATCGACAGAT GATTACATCA CTTATAGTTC TAGTAAATTT ATTAATATAA | 360 |
| AACTCAGAGA CATTCCAATA TCCACATTGC TTACACCATT AGGCATAGAT TCAGTGTCAG | 420 |
| CTATGACAAT TGAAAATGAG CTGTTTTGTG ATTTAAAGGT TTAAATTTCT CTAACCAAAC | 480 |
| TGCTTGATCC AGATGCAGGA CTGCAAATGT TAATATTTGT TCTGGAAGAA CAATCAAATA | 540 |
| AGACTTAAGA GGAAAGGGAA TGGCCACAAT CCACCTGAAA TTTTTTCTTA AAAAGTGTGC | 600 |
| AGCCTACTAA ATCAGAATGA AAATAGAAGT ACAAGATTAT AAACAAAATG CAATCAAACT | 660 |
| TTTCTTAAGC TTACCTAAAG TTATTTCATC TGAAAATTTC AAGCAACTTT GTTCAACATT | 720 |
| AAATTGACAA TCTAAACTAA CAAGTCTTTT GAATTTATGC ATGGTAGTAA ACATTCTCTC | 780 |
| TATTAACTTT ATTACCTAAG GCTAAACCTA AAATTTTTAA GCAAAATTAG AAAAATAGTC | 840 |
| TTCACTCATC AAAAAATAAA GITTGTTACA TTTAGTATTT TCCCAATAAA ATTGGTCGTT | 900 |
| CTTGGTTTTT TATTTGGAGA GTCTGTGCAA AATGTCACTA AAAATAAATT AGCACTAGAA | 960 |
| ATTATTTCTA AATACCAAA | |
| Seq ID NO: 203 DNA Sequence | |
| Nucleic Acid Accession #: NM_005656.1 | |
| Coding sequence: 57 . . . 1535 | |
| 1 11 21 31 41 51 | |
| \| \| \| \| \| \| | |
| GTCATATTGA ACATTCCAGA TACCTATCAT TACTCGATGC TGTTGATAAC AGCAAGATGG | 60 |
| CTTTGAACTC AGGGTCACCA CCAGCTATTG GACCTTACTA TGAAAACCAT GGATACCAAC | 120 |
| CGGAAAACCC CTATCCCGCA CAGCCCACTG TGGTCCCCAC TGTCTACGAG GTGCATCCGG | 180 |
| CTCAGTACTA CCCGTCCCCC GTGCCCCAGT ACGCCCCGAG GGTCCTGACG CAGGCTTCCA | 240 |
| ACCCCGTCGT CTGCACGCAG CCCAAATCCC CATCCGGGAC AGTGTGCACC TCAAAGACTA | 300 |
| AGAAAGCACT GTGCATCACC TTGACCCTGG GGACCTTCCT CGTGGGAGCT GCGCTGGCCG | 360 |
| CTGGCCTACT CTGGAAGTTC ATGGGCAGCA AGTGCTCCAA CTCTGGGATA GAGTGCGACT | 420 |
| CCTCAGGTAC CTCCATCAAC CCCTCTAACT GGTGTGATGG CGTGTCACAC TGCCCCGGCG | 480 |
| GGGASGACGA GAATCGGTGT GTTCGCCTCT ACGGACCAAA CTTCATCCTT CAGATGTACT | 540 |
| CATCTCAGAG GAAGTCCTGG CACCCTGTGT GCCAAGACGA CTGGAACGAG AACTACGGGC | 600 |
| GGGCGGCCTG CAGGGACATG GGCTATAAGA ATAATTTTTA CTCTAGCCAA CGAATAGTGG | 660 |
| ATGACAGCGG ATCCACCAGC TTTATGAAAC TGAACACAAG TGCCGGCAAT GTCGATATCT | 720 |
| ATAAAAAACT GTACCACAGT GATGCCTGTT CTTCAAAAGC AGTGGTTTCT TTACGCTGTT | 780 |
| TACCCTGCCG GGTCAACTTG AACTCAAGCC GCCAGAGCAG GATCGTGGGC GGTGAGAGCG | 840 |
| CGCTCCCGGC GGCCTGGCCC TGGCAGGTCA GCCTGCACGT CCAGAACGTC CACGTGTGCG | 900 |
| GAGGCTCCAT CATCACCCCC GAGTGGATCG TGACAGCCGC CCACTGCGTG GAAAAACCTC | 960 |
| TTAACAATCC ATGGCATTGG ACGGCATTTG CGGGGATTTT GAGACAATCT TTCATGTTCT | 1020 |
| ATGGAGCCGG ATACCAAGTA CAAAAAGTGA TTTCTCATCC AAATTATGAC TCCAAGACCA | 1080 |
| AGAACAATGA CATTGCGCTG ATGAAGCTGC AGAAGCCTCT GACTTTCAAC GACCTAGTGA | 1140 |
| AACCAGTGTG TCTGCCCAAC CCAGGCATGA TGCTGCAGCC AGAACAGCTC TGCTGGATTT | 1200 |
| CCGGGTGGGG GGCCACCGAG GAGAAAGGGA AGACCTCAGA AGTGCTGAAC GCTGCCAAGG | 1260 |
| TGCTTCTCAT TGAGACACAG AGATGCAACA GCAGATATGT CTATGACAAC CTGATCACAC | 1320 |
| CAGCCATGAT CTGTGCCGGC TTCCTGCAGG GGAACGTCGA TTCTTGCCAG GGTGACAGTG | 1380 |
| GAGGGCCTCT CGTCACTTCG AACAACAATA TCTGGTGGCT GATAGGGGAT ACAAGCTGGG | 1440 |
| GTTCTGGCTG TGCCAAAGCT TACAGACCAG GAGTGTACGG GAATGTGATG GTATTCACGG | 1500 |
| ACTGGATTTA TCGACAAATG AAGGCAAACG GCTAATCCAC ATGGTCTTCG TCCTTGACGT | 1560 |
| CGTTTTACAA GAAAACAATG GGGCTGGTTT TGCTTCCCCG TGCATGATTT ACTCTTAGAG | 1620 |
| ATGATTCAGA GGTCACTTCA TTTTTATTAA ACAGTGAACT TGTCTGGCTT TGGCACTCTC | 1680 |
| TGCCATACTG TGCAGGCTGC AGTGGCTCCC CTGCCCAGCC TGCTCTCCCT AACCCCTTGT | 1740 |
| CCGCAAGGGG TGATGGCCGG CTGGTTGTGG GCACTGGCGG TCAATTGTGG AAGGAAGAGG | 1800 |
| GTTGGAGGCT GCCCCCATTG AGATCTTCCT GCTGAGTCCT TTCCAGGGGC CAATTTTGGA | 1860 |
| TGAGCATGGA GCTGTCACTT CTCAGCTGCT GGATGACTTG AGATGAAAAA GGAGAGACAT | 1920 |
| GGAAAGGGAG ACAGCCAGGT GGCACCTGCA GCGGCTGCCC TCTGGGGCCA CTTGGTAGTG | 1980 |
| TCCCCAGCCT ACTTCACAAG GGGATTTTGC TGATGGGTTC TTAGAGCCTT AGCAGCCCTG | 2040 |
| GATGGTGGCC AGAAATAAAG GGACCAGCCC TTCATGGGTG GTGACGTGGT AGTCACTTGT | 2100 |
| AAGGGGAACA GAAACATTTT TGTTCTTATG GGGTGAGAAT ATAGACASTG CCCTTGGTGC | 2160 |
| GAGGGAAGCA ATTGAAAAGG AACTTGCCCT GAGCACTCCT GGTGCAGGTC TCCACCTGCA | 2220 |
| CATTGGGTGG GGCTCCTGGG AGGGAGACTC AGCCTTCCTC CTCATCCTCC CTGACCCTGC | 2280 |
| TCCTAGCACC CTGGAGAGTG AATGCCCCTT GGTCCCTGGC AGGGCGCCAA GTTTGGCACC | 2340 |
| ATGTCGGCCT CTTCAGGCCT GATAGTCATT GGAAATTGAG GTCCATGGGG GAAATCAAGG | 2400 |
| ATGCTCAGTT TAAGGTACAC TGTTTCCATG TTATGTTTCT ACACATTGAT GGTGGTGACC | 2460 |
| CTGAGTTCAA AGCCATCTT | |
| Seq ID NO: 204 Protein Sequence | |
| Protein Accession #: NP 005647.1 | |
| 1 11 21 31 41 51 | |
| \| \| \| \| \| \| | |
| MALNSGSPPA IDPYYENHGY QPENPYPAQP TVVPTVYEVH PAQYYPSPVP QYAPRVLTQA | 60 |
| SNPVVCTQPK SPSGTVCTSK TKKALCITLT LGTFLVGAAL AAGLLWKFMG SKCSNSGIEC | 120 |
| GSSGTCINPS NWCGGVSNCP GGEGENRCVR LYGPNFILGM YSSQRKSWHP VCQGGWNENY | 180 |
| GRAACRGMGY KNNFYSSQGI VGGSGSTSFM KLNTSAGNVG IYKKLYHSGA CSSKAVVSLR | 240 |
| CLACGVNLNS SRQSRIVGGE SALPGAWPWQ VSLNVGNVHV CGGSIITPEW IVTAANCVEK | 300 |
| PLNNPWHWTA FAGILRQSFM FYGAGYQVQK VTSNPNYGSK TKNNGIALMK LQKPLTFNDL | 360 |
| VKPVCLPNPG MMLQPEQLCN ISGWGATEEK GKTSEVLNAA KVLLIETQRC NSRYVYGNLI | 420 |
| TPANICAGFL QGNVGSCQGG SGGPLVTSNN NIWWLIDGTS WGSGCAKAYR PGVYGNVMVF | 480 |
| TGWIYRGMNA NG | |
| Seq ID NO: 205 DNA Sequence | |
| Nucleic Acid Accession #: XM_044533 | |
| Coding sequence: 238 . . . 2751 | |
| 1 11 21 31 41 51 | |
| \| \| \| \| \| \| | |
| GCTCTCCCCA AGCCGAGGCT GCGGGGCCGG CGCCGGCGGC AGGACTGCGG TGCCCCGCGG | 60 |
| AGGGGCTGAC TTTGCCAGGG CCCACTTGAC CCTGTTTCCC ACCTCCCGCC CCCCAGGTCC | 120 |
| GGAGGCGGGG GCCCCCGGGG CGACTCGGGG GCGGACCGCG GGGCGGAGCT GCCGCCCGTG | 180 |
| AGTCCGGCCG AGCCACCTGA GCCCGAGCCG CGGGACACCG TCGCTCCTCC TCTCCGAATG | 240 |
| CTGCGCACCG CGATGGGCCT GAGGAGCTGG CTCGCCGCCC CATGGGGCGC GCTGCCGCCT | 300 |
| CGGCCACCGC TGCTGCTGCT CCTGCTGCTG CTGCTCCTGC TGCAGCCGCC GCCTCCGACC | 360 |
| TGGGCGCTCA GCCCCCGGAT CAGCCTGCCT CTGGGCTCTG AAGAGCGGCC ATTCCTCAGA | 420 |
| TTCGAAGCTG AACACATCTC CAACTACACA GCCCTTCTGC TGAGCAGGGA TGGCAGGACC | 480 |
| CTGTACGTGG GTGCTCGAGA GGCCCTCTTT GCACTCAGTA GCAACCTCAG CTTCCTGCCA | 540 |
| GGCGGGGAGT ACCAGGAGCT GCTTTGGGGT GCAGACGCAG AGAAGAAACA GCAGTGCAGC | 600 |
| TTCAAGGGCA AGGACCCACA GCGCGACTGT CAAAACTACA TCAAGATCCT CCTGCCGCTC | 660 |
| AGCGGCAGTC ACCTGTTCAC CTGTGGCACA GCAGCCTTCA GCCCCATGTG TACCTACATC | 720 |
| AACATGGAGA ACTTCACCCT GGCAAGGGAC GAGAAGGGGA ATGTCCTCCT GGAAGATGGC | 780 |
| AAGGGCCGTT GTCCCTTCGA CCCGAATTTC AAGTCCACTG CCCTGGTGGT TGATGGCGAG | 840 |
| CTCTACACTG GAACAGTCAG CAGCTTCCAA GGGAATGACC CGGCCATCTC GCGGAGCCAA | 900 |
| AGCCTTCGCC CCACCAAGAC CGAGAGCTCC CTCAACTGGC TGCAAGACCC AGCTTTTGTG | 960 |
| GCCTCAGCCT ACATTCCTGA GAGCCTGGGC AGCTTGCAAG GCGATGATGA CAAGATCTAC | 1020 |
| TTTTTCTTCA GCGAGACTGG CCAGGAATTT GAGTTCTTTG AGAACACCAT TGTGTCCCGC | 1080 |
| ATTGCCCGCA TCTGCAAGGG CGATGAGGGT GGAGAGCGGG TGCTACAGCA GCGCTGGACC | 1140 |
| TCCTTCCTCA AGGCCCAGCT GCTGTGCTCA CGGCCCGACG ATGGCTTCCC CTTCAACGTG | 1200 |
| CTGCAGGATG TCTTCACGCT GAGCCCCAGC CCCCAGGACT GGCGTGACAC CCTTTTCTAT | 1260 |
| GGGGTCTTCA CTTCCCAGTG GCACAGGGGA ACTACAGAAG GCTCTGCCGT CTGTGTCTTC | 1320 |
| ACAATGAAGG ATGTGCAGAG AGTCTTCAGC GGCCTCTACA AGGAGGTGAA CCGTGAGACA | 1380 |
| CAGCAGTGGT ACACCGTGAC CCACCCGGTG CCCACACCCC GGCCTGGAGC GTGCATCACC | 1440 |
| AACAGTGCCC GGGAAAGGAA GATCAACTCA TCCCTGCAGC TCCCAGACCG CGTGCTGAAC | 1500 |
| TTCCTCAAGG ACCACTTCCT GATGGACGGG CAGGTCCGAA GCCGCATGCT GCTGCTGCAG | 1560 |
| CCCCAGGCTC GCTACCAGCG CGTGGCTCTA CACCGCGTCC CTGGCCTGCA CCACACCTAC | 1620 |
| GATGTCCTCT TCCTGGGCAC TGGTGACGGC CGGCTCCACA AGGCAGTGAG CGTGGGCCCC | 1680 |
| CGGGTGCACA TCATTGAGGA GCTGCAGATC TTCTCATCGG GACAGCCCGT GCAGAATCTG | 1740 |
| CTCCTGGACA CCCACAGGGG GCTGCTGTAT GCGGCCTCAC ACTCGGGCGT AGTCCAGGTG | 1800 |
| CCCATGGCCA ACTGCAGCCT GTACAGGAGC TGTGGCGACT GCCTCCTCGC CCGGGACCCC | 1860 |
| TACTGTGCTT GGAGCGGCTC CAGCTGCAAG CACGTCAGCC TCTACCAGCC TCAGCTGGCC | 1920 |
| ACCAGGCCGT GGATCCAGGA CATCGAGGGA GCCAGCGCCA AGGACCTTTG CAGCGCGTCT | 1980 |
| TCGGTTGTGT CCCCGTCTTT TGTACCAACA GGGGAGAAGC CATGTGAGCA AGTCCAGTTC | 2040 |
| CAGCCCAACA CAGTGAACAC TTTGGCCTGC CCGCTCCTCT CCAACCTGGC GACCCGACTC | 2100 |
| TGGCTACGCA ACGGGGCCCC CGTCAATGCC TCGGCCTCCT GCCACGTGCT ACCCACTGGG | 2160 |
| GACCTGCTGC TGGTGGGCAC CCAACAGCTG GGGGAGTTCC AGTGCTGGTC ACTAGAGGAG | 2220 |
| GGCTTCCAGC AGCTGGTAGC CAGCTACTGC CCAGAGGTGG TGGAGGACGG GGTGCCAGAC | 2280 |
| CAAACAGATG AGGGTGGCAG TGTACCCGTC ATTATCAGCA CATCGCGTGT GAGTGCACCA | 2340 |
| GCTGGTGGCA AGGCCAGCTG GGGTGCAGAC AGGTCCTACT GGAAGGAGTT CCTGGTGATG | 2400 |
| TGCACGCTCT TTGTGCTGGC CGTGCTGCTC CCAGTTTTAT TCTTGCTCTA CCGGCACCGG | 2460 |
| AACAGCATGA AAGTCTTCCT GAAGCAGGGG GAATGTGCCA GCGTGCACCC CAAGACCTGC | 2520 |
| CCTGTGGTGC TGCCCCCTGA GACCCGCCCA cTCAACGGCC TAGGGCCCCC TAGCACCCCG | 2580 |
| CTCGATCACC GAGGGTACCA GTCCCTGTCA GACAGCCCCC CGGGGTCCCG AGTCTTCACT | 2640 |
| GAGTCAGAGA AGAGGCCACT CAGCATCCAA GACAGCTTCG TGGAGGTATC CCCAGTGTGC | 2700 |
| CCCCGGCCCC GGGTCCGCCT TGGCTCGGAG ATCCGTGACT CTGTGGTGTG AGAGCTGACT | 2760 |
| TCCAGAGGAC GCTGCCCTGG CTTCAGGGGC TGTGAATGCT CGGAGAGGGT CAACTGGACC | 2920 |
| TCCCCTCCGC TCTGCTCTTC GTGGAACACG ACCGTGGTGC CCGGCCCTTG GGAGCCTTGG | 2880 |
| GGCCAGCTGG CCTGCTGCTC TCCAGTCAAG TAGCGAAGCT CCTACCACCC AGACACCCAA | 2940 |
| ACAGCCGTGG CCCCAGAGGT CCTGGCCAAA TATGGGGGCC TGCCTAGGTT GGTGGAACAG | 3000 |
| TGCTCCTTAT GTAAACTGAG CCCTTTGTTT AAAAAACAAT TCCAAATGTG AAACTAGAAT | 3060 |
| GACAGGGAAG AGATAGCATG GCATGCAGCA CACACGGCTG CTCCAGTTCA TGGCCTCCCA | 3120 |
| GGGGTGCTGG GGATGCATCC AAAGTGGTTG TCTGAGACAG AGTTGGAAAC CCTCACCAAC | 3180 |
| TGGCCTCTTC ACCTTCCACA TTATCCCGCT GCCACCGGCT GCCCTGTCTC ACTGCAGATT | 3240 |
| CAGGACCAGC TTGGGCTGCG TGCGTTCTGC CTTGCCAGTC AGCCGAGGAT GTAGTTGTTG | 3300 |
| CTGCCGTCGT CCCACCACCT CAGGGACCAG AGGGCTAGGT TGGCACTGCG GCCCTCACCA | 3360 |
| GGTCCTGGGC TCGGACCCAA CTCCTGGACC TTTCCAGCCT GTATCAGGCT GTGGCCACAC | 3420 |
| GAGAGGACAG CGCGAGCTCA GGAGAGATTT CGTGACAATG TACGCCTTTC CCTCAGAATT | 3480 |
| CAGGGAAGAG ACTGTCGCCT GCCTTCCTCC GTTGTTGCGT GAGAACCCGT GTGCCCCTTC | 3540 |
| CCACCATATC CACCCTCGCT CCATCTTTGA ACTCAAACAC GAGGAACTAA CTCCACCCTG | 3600 |
| GTCCTCTCCC CAGTCCCCAG TTCACCCTCC ATCCCTCACC TTCCTCCACT CTAACGGATA | 3660 |
| TCAACACTGC CCAGCACAGG GGCCCTGAAT TTATGTGGTT TTTATACATT TTTTAATAAG | 3720 |
| ATGCACTTTA TGTCATTTTT TAATAAAGTC TGAAGAATTA CTGTTT | |
| Seq ID NO: 206 Protein Sequence | |
| Protein Accession #: XP_044533.6 | |
| 1 11 21 31 41 51 | |
| \| \| \| \| \| \| | |
| MLRTAMGLRS WLAAPWGALP PRPPLLLLLL LLLLLQPPPP TWALSPRISL PLGSEERPFL | 60 |
| RFEAEHISNY TALLLSRGGR TLYVGAREAL FALSSNLSFL PGGEYQELLW CAGAEKKQQC | 120 |
| SEKGKGPGRG CQNYIKILLP LSGSHLFTCG TAAFSPMCTY INMENETLAR GEKCNVLLEG | 180 |
| GKGRCPFGPN FKSTALVVGG ELYTGTVSSF QGNGPAISRS QSLRPTKTES SLNWLQGPAF | 240 |
| VASAYIPESL GSEQGGGGKI YFFFSETGQE FEFPENTIVS RIARICKGGE GGERVLQQRW | 300 |
| TSPLKAQLLC SEPGGGNPEN VLQGVFTLSP SPQGWRGTLF YGVFTSQWHR GTTEGSAVCV | 360 |
| FTMKGVQRVF SCLYKEVNRE TQQWYTVTNP VPTPRPGACI TNSARERKIN SSLQLPGRVL | 420 |
| NPLKGHFLMG CQVRSRMLLL QPQARYQRVA VHRVPGLHMT YGVLFLGTGG GRLHKAVSVC | 480 |
| PRVNIIEELQ IFSSCQPVQN LLLGTHRGLL YAASHSGVVQ VPMANCSLYR SCGGCLLARG | 540 |
| PYCAWSCSSC KHVSLYQPQL ATRPWIQGIE GASAKGLCSA SSVVSPSPVP TGEKPCEQVQ | 600 |
| FQPNTVNTLA CPLLSNLATR LWLRNCAPVN ASASCHVLPT GGLLLVGTQQ LGEFQCWSLE | 660 |
| EGPQQLVASY CPEVVEGGVA GQTGEGGSVP VIISTSRVSA PAGGKASWGA GRSYWKEFLV | 720 |
| MCTLPVLAVL LPVLFLLYRH RNSMKVFLKQ CSCASVHPKT CPVVLPPETR PLNGLGPPST | 780 |
| PLGNRGYQSL SGSPPGSRVF TESEKRPLSI QGSFVEVSPV CPRPRVRLGS EIRGSVV | |

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

What is claimed is:

1. A method of detecting a bladder cancer-associated transcript in a cell from a patient, the method comprising contacting a biological sample from the patient with a polynucleotide that selectively hybridizes to a sequence at least 80% identical to a sequence as shown in Tables 1A-13.

2. The method of claim 1, wherein the biological sample comprises isolated nucleic acids.

3. The method of claim 2:
a) wherein the nucleic acids are mRNA; or
b) further comprising the step of amplifying nucleic acids before the step of contacting the biological sample with the polynucleotide.

4. The method of claim 1, wherein the polynucleotide:
a) comprises a sequence as shown in Tables 1A-13; or
b) is immobilized on a solid surface.

5. The method of claim 1, wherein the patient is:
a) undergoing a therapeutic regimen to treat bladder cancer; or
b) suspected of having bladder cancer.

6. An isolated nucleic acid molecule consisting of a polynucleotide sequence as shown in Tables 1A-13.

7. The nucleic acid molecule of claim 6, which is labeled.

8. An expression vector comprising the nucleic acid of claim 7.

9. A host cell comprising the expression vector of claim 8.

10. An isolated polypeptide which is encoded by a nucleic acid molecule having polynucleotide sequence as shown in Tables 1A-13.

11. An antibody that specifically binds a polypeptide of claim 10.

12. The antibody of claim 11, further conjugated to an effector component.

13. The antibody of claim 12, wherein the effector component is a fluorescent label.

14. The antibody of claim 12, wherein the effector component is a radioisotope or a cytotoxic chemical.

15. The antibody of claim 11, which is
a) an antibody fragment; or
b) a humanized antibody

16. A method of detecting a bladder cancer cell in a biological sample from a patient, the method comprising contacting the biological sample with an antibody of claim 11.

17. The method of claim 16, wherein the antibody is further conjugated to an effector component.

18. The method of claim 17, wherein the effector component is a fluorescent label.

19. A method for identifying a compound that modulates a bladder cancer-associated polypeptide, the method comprising the steps of:
a) contacting the compound with a bladder cancer-associated polypeptide, the polypeptide encoded by a polynucleotide that selectively hybridizes to a sequence at least 80% identical to a sequence as shown in Tables 1A-13; and
b) determining the functional effect of the compound upon the polypeptide.

20. A drug screening assay comprising the steps of
a) administering a test compound to a mammal having bladder cancer or a cell isolated therefrom;
b) comparing the level of gene expression of a polynucleotide that selectively hybridizes to a sequence at least 80% identical to a sequence as shown in Tables 1A-13 in a treated cell or mammal with the level of gene expression of the polynucleotide in a control cell or mammal, wherein a test compound that modulates the level of expression of the polynucleotide is a candidate for the treatment of bladder cancer.
